(19) United States
(12) Patent Application Publication
(10) Pub. No.: US 2024/0254138 A1
(43) Pub. Date: Aug. 1, 2024

(54) TRICYCLIC PYRIMIDINES AS CYCLIN-DEPENDENT KINASE 7 (CDK7) INHIBITORS

(71) Applicant: JANSSEN PHARMACEUTICA NV, Beerse (BE)

(72) Inventors: Ian STANSFIELD, Saint-Germain-en-Laye (FR); Yannick Aimé Eddy LIGNY, Sotteville-les-Rouen (FR); Yvan René Ferdinand SIMONNET, Princeton, NJ (US); Nathalie Claudie Isabelle AMBLARD, Crestot (FR); James Patrick EDWARDS, Ambler, PA (US)

(21) Appl. No.: 18/549,638

(22) PCT Filed: Mar. 8, 2022
(86) PCT No.: PCT/EP2022/055808

§ 371 Date: Sep. 8, 2023

(30) Foreign Application Priority Data
Mar. 9, 2021 (EP) 21161543.0

(51) Int. Cl. C07D 498/04; A61P 35/02; C07D 498/14; C07D 519/00

(52) U.S. Cl. CPC C07D 498/04; A61P 35/02; C07D 498/14; C07D 519/00

(57) ABSTRACT

The invention relates to pharmaceutical compounds of formula (I) and pharmaceutical compositions comprising said compounds, to processes for the preparation of said compounds and to the use of said compounds as inhibitors of cyclin-dependent kinase 7 (CDK7) and to their use in the treatment of diseases, e.g., cancer.

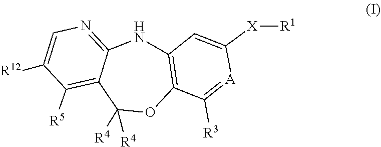

### CROSS-REFERENCE

This application claims benefit of EP Application No. 21161543, filed on Mar. 9, 2021, which is herein incorporated by reference in its entirety.

### FIELD OF THE INVENTION

The invention relates to pharmaceutical compounds and pharmaceutical compositions comprising said compounds, to processes for the preparation of said compounds and to the use of said compounds as inhibitors of cyclin-dependent kinase 7 (CDK7) and to their use in the treatment of diseases, e.g., cancer.

### BACKGROUND OF THE INVENTION

The members of the cyclin-dependent kinase (CDK) family play critical regulatory roles in proliferation. Unique among the mammalian CDKs, CDK7 has consolidated kinase activities, regulating both the cell cycle and transcription. In the cytosol, CDK7 exists as a heterotrimeric complex and is believed to function as a CDK1/2-activating kinase (CAK), whereby phosphorylation of conserved residues in CDK1/2 by CDK7 is required for full catalytic CDK activity and cell cycle progression. In the nucleus, CDK7 forms the kinase core of the RNA polymerase (RNAP) II general transcription factor complex and is charged with phosphorylating the C-terminal domain (CTD) of RNAP II, a requisite step in gene transcriptional initiation. Together, the two functions of CDK7, i.e., CAK and CTD phosphorylation, support critical facets of cellular proliferation, cell cycling, and transcription.

Disruption of RNAP II CTD phosphorylation has been shown to preferentially affect proteins with short half-lives, including those of the anti-apoptotic BCL-2 family.

Cancer cells have demonstrated ability to circumvent pro-cell death signaling through upregulation of BCL-2 family members. Therefore, inhibition of human CDK7 kinase activity is likely to result in anti-proliferative activity.

The discovery of selective inhibitors of CDK7 has been hampered by the high sequence and structural similarities of the kinase domain of CDK family members. Therefore, there is a need for the discovery and development of selective CDK7 inhibitors. Such CKD7 inhibitors hold promise as therapeutic agents for the treatment of chronic lymphocytic leukemia and other cancers.

### SUMMARY OF THE INVENTION

The present invention relates to a compound of formula (I), including any tautomeric and stereochemically isomeric form, isotopically labeled derivative, or a pharmaceutically acceptable salt or solvate thereof:

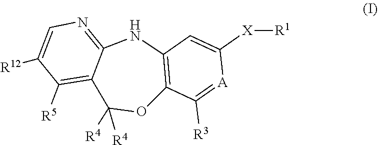

wherein,
X is a 5-6 membered non-aromatic heterocycle; —NH—C(O)—; —NH—CH₂—; —CH₂—; —CH₂—CH₂—; —CH—CH—; absent; a pyridine; a pyrimidine; a 4-7 membered non-aromatic heterocycle; a 4-10 membered non-aromatic bridged heterocycle; C₃₋₇cycloalkyl; or C₅₋₇cycloalkenyl; wherein each of the cycles, independently, may be optionally substituted with —C₁₋₃alkyl, halo, or hydroxy;
R¹ is a 4-5 membered non-aromatic heteromonocycle or a 4-9 membered non-aromatic heteromonocycle, heterobicycle, or spiro-heterobicycle having at least one nitrogen atom, wherein the at least one nitrogen atom is substituted with —C(═O)—CH═CH—R⁶, or —C(═O)—CH—CH—R⁷, and wherein the 4-5 or 4-9 membered non-aromatic heterocycle is optionally substituted with C₁₋₃alkyl, halo, or D; or R¹ is phenyl or pyridine, each independently, substituted with —NR¹¹—C(═O)—CH═CH—R⁶, or —NR¹¹—C(═O)—CH—CH—R⁷, and said phenyl or pyridine is optionally substituted with C₂₋₅alkenyl, C₂₋₅alkynyl, or —O—C₂₋₅alkenyl; or R¹ is C₁₋₃alkyl substituted with —NH—C(═O)—CH═CH—R⁶ or —NH—C(═O)—CH—CH—R⁷;
A is a CR² or N;
R² is H, C₁₋₃alkyl, cyano, halo, or C₂₋₃alkynyl;
R³ is C₁₋₃alkyl, H, halogen, C₂₋₃alkenyl, C₂₋₃alkynyl, cyano, C₃₋₇cycloalkyl; C₁₋₃alkyl substituted with one, two, or three halo, hydroxy, carboxyl, amino, mono- or di(C₁₋₆alkyl)amino; or 1-imidazolyl, 2-imidazolyl, 4-imidazolyl;
R⁴ is, each independently, hydrogen; methyl; C₁₋₃alkyl; C₁₋₃alkyl substituted with one, two, or three halo;
R⁵ is 4-morpholinyl, 4-tetrahydropyranyl, 4-pyrazolyl, a 4-7 membered saturated or partially unsaturated heterocycle, a 5-6 membered heteroaryl, or a 6-12 membered spiro-bicyclic heterocycle; wherein each of the cycles have one, two, or three heteroatoms selected from sulphur, nitrogen, and oxygen; and wherein,
   said sulphur, if present, is substituted with dioxo, or with oxo and imino;
   said one, two, or three nitrogens, if present, may, each independently, be optionally substituted with C₁₋₃alkyl;
   any one of the carbon atoms of the cycles may be optionally substituted with C₁₋₃alkyl, hydroxyC₁₋₃alkyl, C₁₋₃alkoxy, oxo, C₁₋₃alkylsulfonyl, cyano, hydroxy, halo, carboxyl, mono- or di(C₁₋₆alkyl)amino, polyhaloC₁₋₃alkyl, polyhaloC₁₋₃alkoxy, C₂₋₃alkenyl, and C₂₋₃alkynyl;
R⁶ is H; —C₁₋₃alkyl optionally substituted with one, two, or three substituents selected from halo, D, 4-morpholinyl, and —NR^{7a}R^{7b}; wherein each of R^{7a} and R^{7b} is, independently, C₁₋₃alkyl, C₂₋₄alkenyl, or C₂₋₄alkynyl; or R^{7a} and R^{7b} taken together form a heterocycle;
R⁷ is —C₁₋₃alkyl optionally substituted with one, two, or three substituents selected from halo, D, and —NR^{7a}R^{7b}; wherein each of R^{7a} and R^{7b} is, independently, C₁₋₃alkyl, C₂₋₄alkyl, or C₂₋₄alkyl; or R^{7a} and R^{7b} taken together form a heterocycle;
R¹¹ is C₂₋₅alkenyl or C₂₋₅alkynyl; and
R¹² is hydrogen, halo, methyl, or cyano.

The present invention relates to a compound as defined above, including any tautomeric and stereochemically isomeric form, isotopically labeled derivative, or a pharmaceutically acceptable salt or solvate thereof,
wherein,
X is a 4-7 membered non-aromatic heterocycle, a 4-10 membered non-aromatic bridged heterocycle, C₄₋₇cycloalkyl, C₅₋₇cycloalkenyl; wherein each of the cycles, independently, may be optionally substituted with —C₁₋₃alkyl;
R¹ is a 4-7 membered non-aromatic heterocycle having at least one nitrogen atom, wherein the at least one nitrogen atom is substituted with —C(═O)—CH═CH—R⁶, or —C(═O)—CH—CH—R⁷, and wherein the 4-7 membered non-aromatic heterocycle is optionally substituted with C₁₋₃alkyl, halo, or D; or R¹ is C₁₋₃alkyl substituted with —NH—C(═O)—CH═CH—R⁶ or —NH—C(═O)—CH—CH—R⁷;
A is a CR² or N;
R² is H, C₁₋₃alkyl, or cyano;
R³ is C₁₋₃alkyl, H, halogen, cyano, C₃₋₇cycloalkyl; or C₁₋₃alkyl substituted with one, two, or three halo;
R⁴ is, each independently, hydrogen or methyl;
R⁵ a 4-7 membered saturated or partially unsaturated heterocycle, a 5-6 membered heteroaryl, or a 6-12 membered spiro-bicyclic heterocycle; wherein each of the cycles have one, two, or three heteroatoms selected from sulphur, nitrogen, and oxygen; and wherein,
   said sulphur, if present, is substituted with dioxo, or with oxo and imino;
   said one, two, or three nitrogens, if present, may, each independently, be optionally substituted with C₁₋₃alkyl;
   any one of the carbon atoms of the cycles may be optionally substituted with C₁₋₃alkyl, hydroxyC₁₋₃alkyl, C₁₋₃alkoxy, oxo, C₁₋₃alkylsulfonyl, cyano, hydroxy, halo, carboxyl, mono- or di(C₁₋₆alkyl)amino, polyhaloC₁₋₃alkyl, polyhaloC₁₋₃alkoxy, C₂₋₃alkenyl, and C₂₋₃alkynyl;
R⁶ is H; —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle;
R⁷ is —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle; and
R¹² is hydrogen.

The present invention relates to a compound as defined above, including any tautomeric and stereochemically isomeric form, isotopically labeled derivative, or a pharmaceutically acceptable salt or solvate thereof,
wherein,
X is a 5-6 membered non-aromatic heterocycle optionally substituted with —C₁₋₃alkyl;
R¹ is a 4-5 membered non-aromatic heterocycle having at least one nitrogen atom, wherein the at least one nitrogen atom is substituted with —C(═O)—CH═CH—R⁶, or —C(═O)—CH—CH—R⁷, and wherein the 4-5 membered non-aromatic heterocycle is optionally substituted with C₁₋₃alkyl, halo, or D;
A is a CR² or N;
R² is H, C₁₋₃alkyl, or cyano;
R³ is C₁₋₃alkyl, H, halogen, cyano, C₃₋₇cycloalkyl; or C₁₋₃alkyl substituted with one, two, or three halo;
R⁴ is, each independently, hydrogen or methyl;
R⁵ a 4-morpholinyl, 4-tetrahydropyranyl, or 4-pyrazolyl;
R⁶ is H; —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle;
R⁷ is —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle; and
R¹² is hydrogen.

The present invention relates to a compound as defined above, wherein the compound is of formula (II), including any tautomeric and stereochemically isomeric form, isotopically labeled derivative, or a pharmaceutically acceptable salt or solvate thereof,

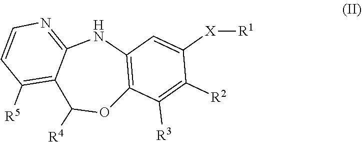

wherein each of X, R¹, R², R³, R⁴, and R⁵, independently, is as defined herein. The present invention relates to a compound as defined above, wherein the compound is of formula (IIa), (IIb), (IIc), (IId), (IIe), or (IIf), including any tautomeric and stereochemically isomeric form, isotopically labeled derivative, or a pharmaceutically acceptable salt or solvate thereof:

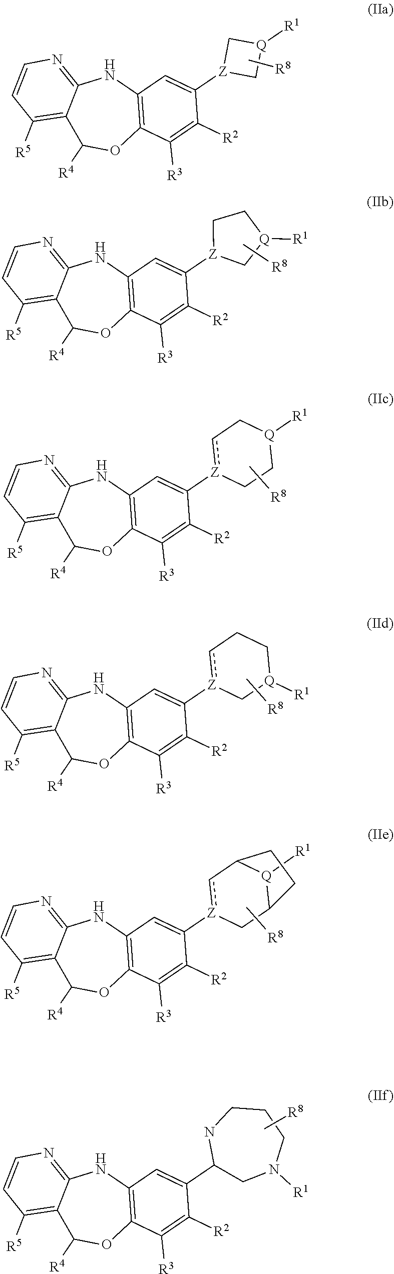

wherein in each of the compounds of formula (IIa), (IIb), (IIc), (IId), (IIIe), or (IIf),
each Q is, independently, CH or N;
each Z is, independently, CH or N;
each of R¹, R², R³, R⁴, and R⁵, independently, is as defined herein;
each R⁸ is, independently, H or —C₁₋₃alkyl; and said RY may be bound to any carbon or nitrogen atom of the cycle; and
each dashed bond is, independently, an optional double bond.

The present invention relates to a compound as defined above, wherein the compound is of formula (IIIa), (IIIb), (IIIc), (IIId), (IIIe), or (IIIf), including any tautomeric and stereochemically isomeric form, isotopically labeled derivative, or a pharmaceutically acceptable salt or solvate thereof:

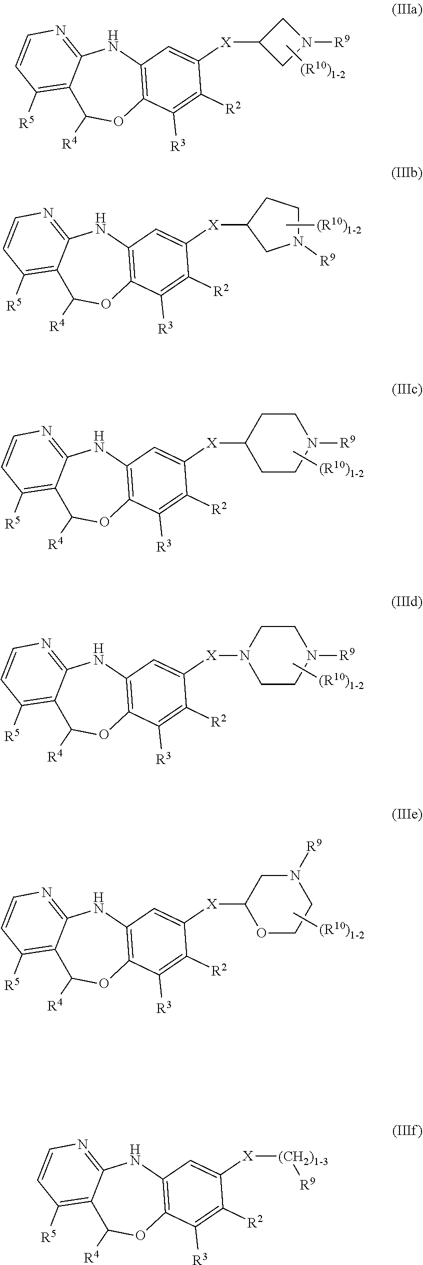

wherein
each R⁹ is, independently, —C(═O)—CH═CH—R⁶, or —C(═O)—CH—CH—R⁷;
each R¹⁰ is, independently, H, —C₁₋₃alkyl, halo, or D; and said R¹⁰ may be bound to any carbon atom of the cycle; and
each of R², R³, R⁴, R⁵, R⁶, and R⁷, independently, is as defined herein.

The present invention relates to a compound as defined above, wherein the compound is of formula (IVa), (IVb), (IVc), (IVd), (IVe), (IVf), (IVg), (IVh), (IVi), (IVj), (IVk), (IVl), (IVm), (IVn), (Ivo), (IVp), or (IVq), including any tautomeric and stereochemically isomeric form, isotopically labeled derivative, or a pharmaceutically acceptable salt or solvate thereof:

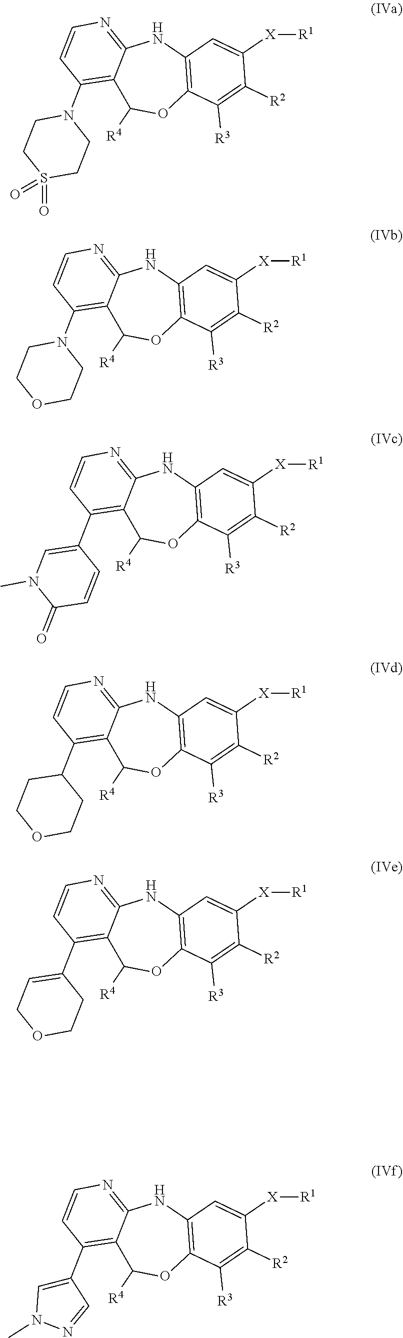

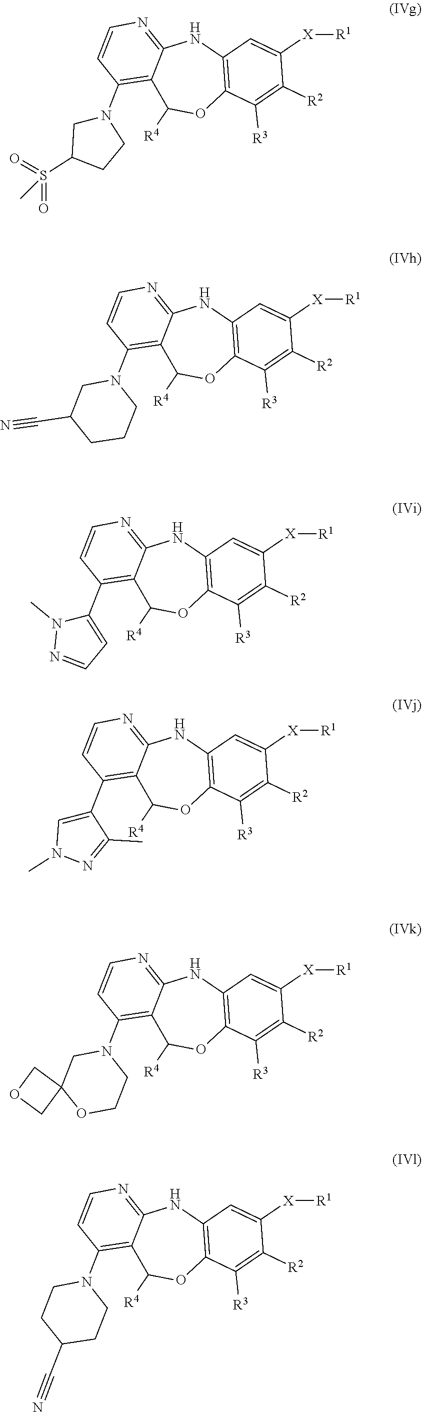

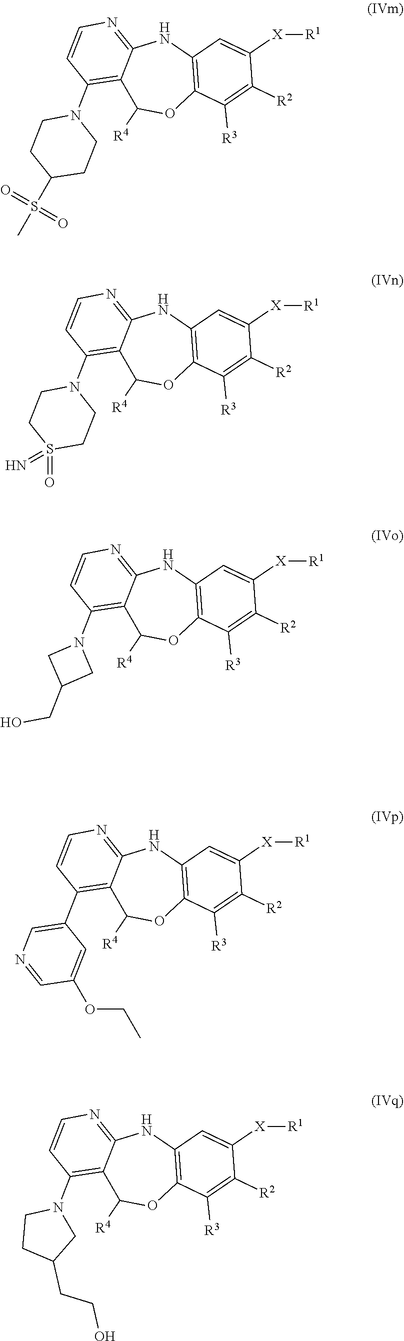

wherein
each of X, R¹, R², R³, and R⁴, independently, is as defined herein.

The present invention relates to a compound as defined herein, wherein the compound is of formula (Va) or (Vb), including any tautomeric and stereochemically isomeric form, isotopically labeled derivative, or a pharmaceutically acceptable salt or solvate thereof.

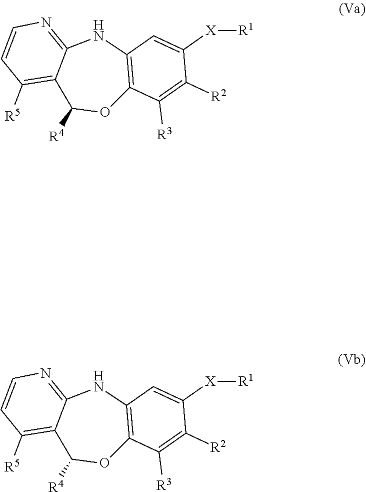

wherein,
each of X, R¹, R², R³, R⁴, and R⁵, independently, is as defined herein.

The present invention relates to a compound of formula (VI), including any tautomeric and stereochemically isomeric form, isotopically labeled derivative, or a pharmaceutically acceptable salt or solvate thereof,

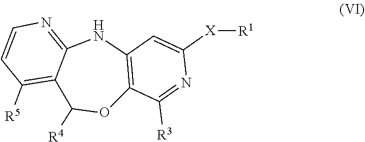

wherein each of X, R¹, R³, R⁴, and R⁵, independently, is as defined herein.

The present invention relates to acompound of formula (VI), including any tautomeric and stereochemically isomeric form, isotopically labeled derivative, or a pharmaceutically acceptable salt or solvate thereof,
wherein,
X is a 4-7 membered non-aromatic heterocycle;
R¹ is a 4-7 membered non-aromatic heterocycle having at least one nitrogen atom, wherein the at least one nitrogen atom is substituted with —C(═O)—CH═CH—R⁶, or —C(═O)—CH—CH—R⁷;
R³ is C₁₋₃alkyl, H, halogen, cyano, C₃₋₇cycloalkyl; or C₁₋₃alkyl substituted with one, two, or three halo;
R⁴ is methyl or H;
R⁵ is a 4-7 membered saturated or partially unsaturated heterocycle, a 5-6 membered heteroaryl, or a 6-12 membered spiro-bicyclic heterocycle; wherein each of the cycles have one, two, or three heteroatoms selected from sulphur, nitrogen, and oxygen; and wherein,
   said sulphur, if present, is substituted with dioxo, or with oxo and imino;
   said one, two, or three nitrogens, if present, may, each independently, be optionally substituted with C₁₋₃alkyl;
   any one of the carbon atoms of the cycles may be optionally substituted with C₁₋₃alkyl, hydroxyC₁₋₃alkyl, C₁₋₃alkoxy, oxo, C₁₋₃alkylsulfonyl, cyano, hydroxy, halo, carboxyl, mono- or di(C₁₋₆alkyl)amino, polyhaloC₁₋₃alkyl, polyhaloC₁₋₃alkoxy, C₂₋₃alkenyl, and C₂₋₃alkynyl;
R⁶ is H; —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle; and
R⁷ is —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle.

The present invention relates to a compound of formula (VIIa), (VIIb), (VIIc), (VIId), (VIIe), or (VIIf), including any tautomeric and stereochemically isomeric form, isotopically labeled derivative, or a pharmaceutically acceptable salt or solvate thereof:

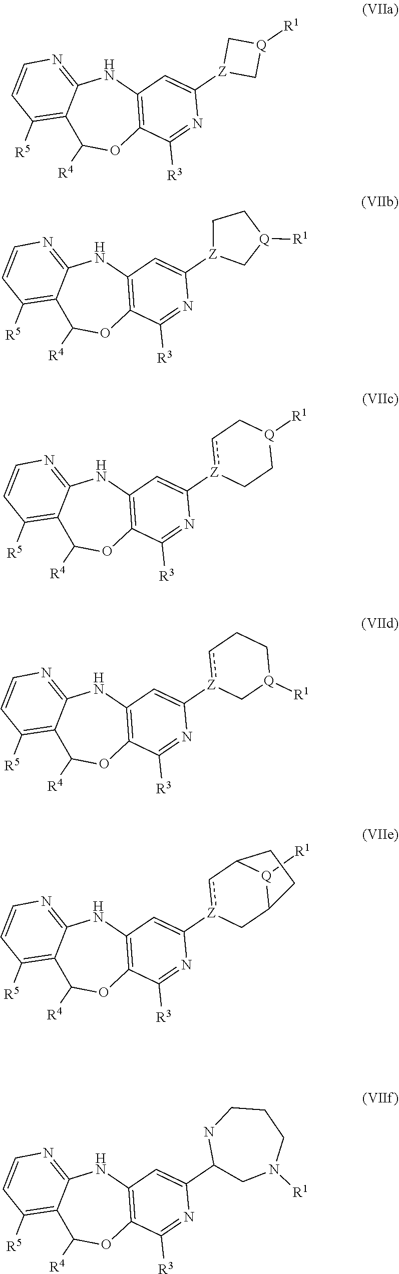

wherein,
each Q is, independently, CH or N;
each Z is, independently, CH or N;
each of R¹, R³, R⁴, and R⁵, independently, is as defined herein.

The present invention relates to a compound of formula (VIIIa), (VIIIb), (VIIIc), (VIIId), (VIIIe), or (VIIIf) including any tautomeric and stereochemically isomeric form, isotopically labeled derivative, or a pharmaceutically acceptable salt or solvate thereof:

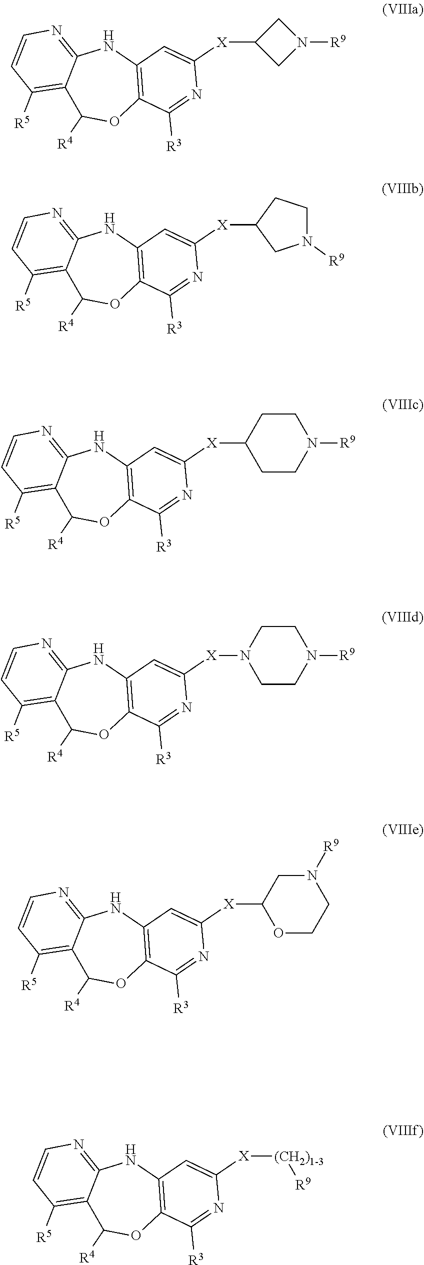

wherein
R⁹ is —C(═O)—CH═CH—R⁶, or —C(═O)—CH—CH—R⁷; and
each of X, R³, R⁴, R⁵, R⁶, and R⁷, independently, is as defined herein.

The present invention relates to a compound of formula (IXa), (IXb), (IXc), (IXd), (IXe), (IXf), (IXg), (IXh), (IXi), (IXj), (IXk), (IXl), (IXm), (IXn), (IXo), (IXp), or (IXq), including any tautomeric and stereochemically isomeric form, isotopically labeled derivative, or a pharmaceutically acceptable salt or solvate thereof:

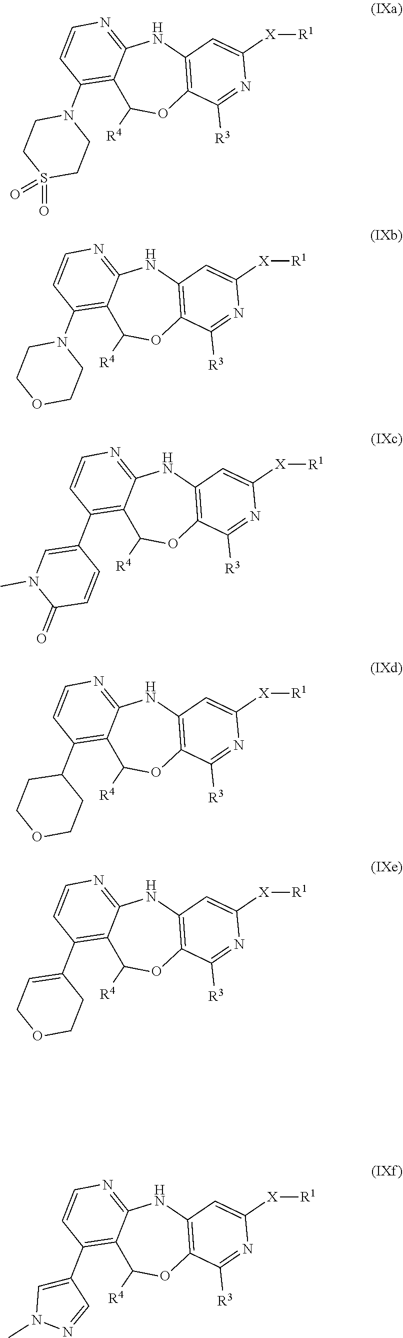

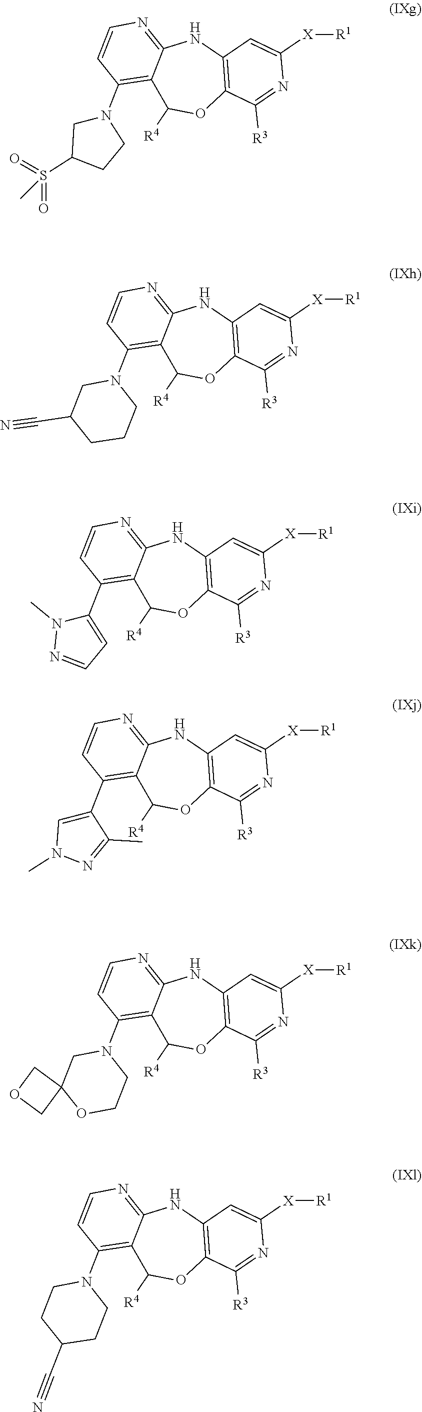

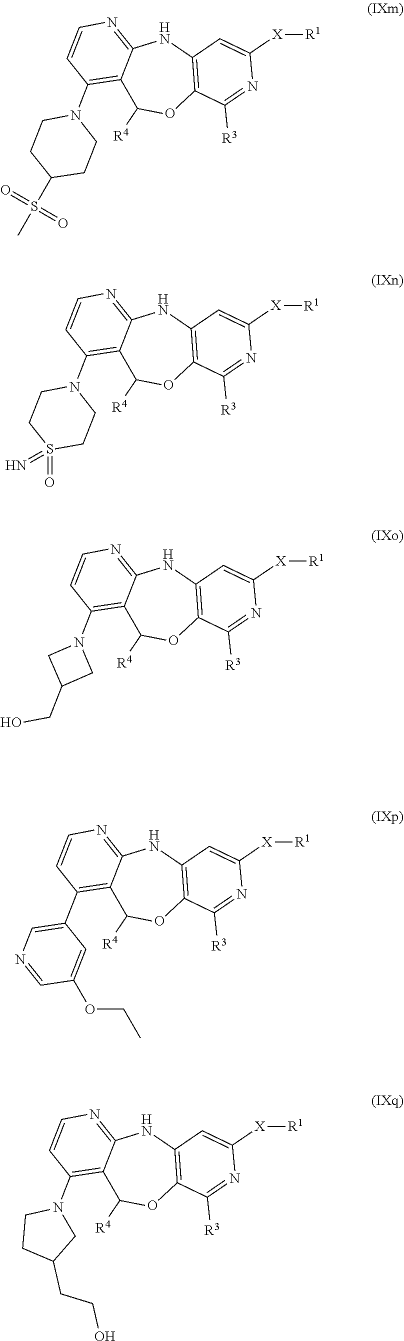

wherein,
each of X, R¹, R³, and R⁴, independently, is as defined herein.

The present invention relates to a compound of formula (VI), including any tautomeric and stereochemically isomeric form, isotopically labeled derivative, or a pharmaceutically acceptable salt or solvate thereof:
wherein
R⁵ in

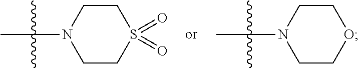 [SMILES: *N1CCS(=O)(=O)CC1 ; *N1CCOCC1]

each of X, R¹, R³, and R⁴, independently, is as defined herein.

The present invention relates to a compound of formula (Xa) or (Xb), including any tautomeric and stereochemically isomeric form, isotopically labeled derivative, or a pharmaceutically acceptable salt or solvate thereof:

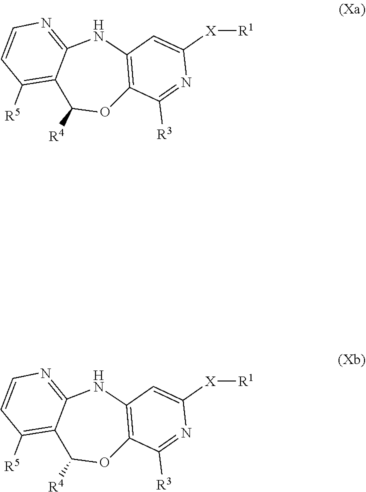

wherein,
each of X, R¹, R³, R⁴, and R⁵, independently, is as defined herein.

The present invention particularly relates to a compound, including any tautomeric and stereochemically isomeric form, isotopically labeled derivative, or a pharmaceutically acceptable salt or solvate thereof, wherein the compound is selected from the compounds listed in the claims.

The present invention further relates to a pharmaceutical composition comprising a compound as disclosed herein, and a pharmaceutically acceptable carrier.

The present invention further relates to any compound disclosed herein, for use in therapy.

The present invention further relates to any compound disclosed herein, for use in the prophylaxis and/or treatment of a disease state or condition mediated by a cyclin-dependent kinase 7 (CDK7).

The present invention further relates to any of the compounds for the use disclosed herein above, wherein the disease state or condition mediated by CDK7 is a proliferative disease selected from cancer, leukemia, acute myeloid leukemia (AML), chronic myelogenous leukemia (CML), lymphoma, B cell lymphoma, chronic lymphocytic leukemia (CLL), acute lymphoblastic leukemia (ALL), T-cell acute lymphoblastic leukemia (T-ALL), Hodgkin's lymphoma, non-Hodgkin's lymphoma, melanoma, multiple myeloma, bone cancer, osteosarcoma, Ewing's sarcoma, breast cancer, triple-negative breast cancer (TNBC), brain cancer, neuroblastoma, lung cancer, small cell lung cancer (SCLC), large cell lung cancer, benign neoplasm, angiogenesis, an inflammatory disease, rheumatoid arthritis, an autoinflammatory disease, or an autoimmune disease.

The present invention also relates to the use of any compound disclosed herein for the manufacture of a medicament for the prophylaxis or treatment of a proliferative disease.

The proliferative disease may be cancer, leukemia, acute myeloid leukemia (AML), chronic myelogenous leukemia (CML), lymphoma, B cell lymphoma, chronic lymphocytic leukemia (CLL), acute lymphoblastic leukemia (ALL), T-cell acute lymphoblastic leukemia (T-ALL), Hodgkin's lymphoma, non-Hodgkin's lymphoma, melanoma, multiple myeloma, bone cancer, osteosarcoma, Ewing's sarcoma, breast cancer, triple-negative breast cancer (TNBC), brain cancer, neuroblastoma, lung cancer, small cell lung cancer (SCLC), large cell lung cancer, benign neoplasm, angiogenesis, an inflammatory disease, rheumatoid arthritis, an autoinflammatory disease, or an autoimmune disease.

The present invention relates as well to a method for the prophylaxis or treatment of a disease state or condition mediated by a CDK7, which method comprises administering to a subject in need thereof an effective amount of a compound as disclosed herein. The disease or condition is selected from a proliferative disease, cancer, leukemia, acute myeloid leukemia (AML), chronic myelogenous leukemia (CML), lymphoma, B cell lymphoma, chronic lymphocytic leukemia (CLL), acute lymphoblastic leukemia (ALL), T-cell acute lymphoblastic leukemia (T-ALL), Hodgkin's lymphoma, non-Hodgkin's lymphoma, melanoma, multiple myeloma, bone cancer, osteosarcoma, Ewing's sarcoma, breast cancer, triple-negative breast cancer (TNBC), brain cancer, neuroblastoma, lung cancer, small cell lung cancer (SCLC), large cell lung cancer, benign neoplasm, angiogenesis, an inflammatory disease, rheumatoid arthritis, an autoinflammatory disease, or an autoimmune disease.

The subject may be a mammal.

The present invention also relates to an in vitro method of modulating CDK7 activity comprising contacting the CDK7 protein, or a portion thereof, with a compound as disclosed herein.

### INCORPORATION BY REFERENCE

All publications, patents, patent applications, and published nucleotide and amino acid sequences (e.g., sequences available in GenBank or other databases) mentioned in this specification are herein incorporated by reference to the same extent as if each individual publication, patent, patent application, or published nucleotide and amino acid sequence, was specifically and individually indicated to be incorporated by reference.

### DETAILED DESCRIPTION OF THE INVENTION

### Definitions

Unless defined otherwise, all technical and scientific terms used herein have the same meaning as is commonly understood to which the claimed subject matter belongs. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed.

In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions and acceptable error margins, for such given value.

As used herein, the expression “one or more” refers to at least one, for example one, two, three, four, five or more, whenever possible and depending on the context. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4^{th} Ed.” Vols. A (2000) and B (2001), Plenum Press, New York.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein. Hereinbefore and hereinafter, the term “compound of formula (I)” is meant to include the addition salts, the solvates and the stereoisomers thereof.

As used herein, “C_{x-y}” (where x and y are integers) refers to the number of carbon atoms that make up the moiety to which it designates (excluding optional substituents). Thus, a C₁₋₆alkyl group contains from 1 to 6 carbon atoms, a C₃₋₆cycloalkyl group contains from 3 to 6 carbon atoms, a C₁₋₄alkoxy group contains from 1 to 4 carbon atoms, and so on.

The term “halo” or, alternatively, “halogen” means fluoro, chloro, bromo and iodo.

The “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁₋₆alkyl” or similar designations.

By way of example, the term “C₁₋₄alkyl”, or “C₁₋₆alkyl” as used herein as a group or part of a group refers to a linear or branched saturated hydrocarbon group containing from 1 to 4 or 1 to 6 carbon atoms, respectively. Examples of such groups include methyl, ethyl, n-propyl, isopropyl, n-butyl, isobutyl, sec-butyl, tert-butyl, n-pentyl, isopentyl, neopentyl, hexyl, and the like.

The term “alkenyl” refers to a type of alkyl group in which at least two atoms of the alkyl group form a double bond that is not part of an aromatic group. Non-limiting examples of an alkenyl group include —CH═CH₂, —C(CH₃)═CH₂, —CH═CHCH₃, —CH═C(CH₃)₂ and —C(CH₃)═CHCH₃. The alkenyl moiety may be branched or a straight chain. Alkenyl groups may have 2 to 6 carbons. Alkenyl groups can be substituted or unsubstituted. Depending on the structure, an alkenyl group can be a monoradical or a diradical (i.e., an alkenylene group). Examples of “alkenyl” include also “C₂₋₄alkenyl” or “C₂₋₆alkenyl”.

The term “alkynyl” refers to a type of alkyl group in which at least two atoms of the alkyl group form a triple bond. Non-limiting examples of an alkynyl group include —C≡CH, —C≡CCH₃, —C≡CCH₂CH₃ and —C≡CCH₂CH₂CH₃. The alkynyl moiety may be branched or a straight chain. An alkynyl group can have 2 to 6 carbons. Alkynyl groups can be substituted or unsubstituted. Depending on the structure, an alkynyl group can be a monoradical or a diradical (i.e., an alkynylene group). Examples of “alkynyl” include also “C₂₋₄alkynyl” or “C₂₋₆alkynyl”.

An “alkoxy” refers to a “—O-alkyl” group, where alkyl is as defined herein.

The term “C₁₋₄alkoxy” or “C₁₋₆alkoxy” as used herein as a group or part of a group refers to an —O—C₁₋₄alkyl group or an —O—C₁₋₆alkyl group wherein C₁₋₄alkyl and C₁₋₆alkyl are as defined herein. Examples of such groups include methoxy, ethoxy, propoxy, butoxy, and the like.

The term “hydroxyC₁₋₄alkyl” or “hydroxyC₁₋₆alkyl” as used herein as a group or part of a group refers to a C₁₋₄alkyl or C₁₋₆alkyl group as defined herein wherein one or more than one hydrogen atoms are replaced with a hydroxyl group. The terms “hydroxyC₁₋₄alkyl” or “hydroxyC₁₋₆alkyl” therefore include monohydroxyC₁₋₄alkyl, monohydroxyC₁₋₆alkyl and also polyhydroxyC₁₋₄alkyl and polyhydroxyC₁₋₆alkyl. There may be one, two, three or more hydrogen atoms replaced with a hydroxyl group, so the hydroxyC₁₋₄alkyl or hydroxyC₁₋₆alkyl may have one, two, three or more hydroxyl groups. Examples of such groups include hydroxymethyl, hydroxyethyl, hydroxypropyl and the like.

The term “haloalkyl” refers to an alkyl group as defined herein wherein one or more than one hydrogen atom is replaced with one or more halogens. The term “haloalkyl” includes “haloC₁₋₄alkyl”, “haloC₁₋₆alkyl”, monohaloC₁₋₄alkyl, monohaloC₁₋₆alkyl, polyhaloC₁₋₄alkyl, and polyhaloC₁₋₆alkyl. There may be one, two, three or more hydrogen atoms replaced with a halogen, so the haloC₁₋₄alkyl or haloC₁₋₆alkyl may have one, two, three or more halogens. The halogens may the same or they may be different. Non-limiting examples of haloalkyls include —CH₂Cl, —CF₃, —CHF₂, —CH₂CF₃, —CF₂CF₃, —CF(CH₃)₂, fluoroethyl, fluoromethyl, trifluoroethyl, and the like.

The term “heteroalkyl” refers to an alkyl radical where one or more skeletal chain atoms is selected from an atom other than carbon, e.g., oxygen, nitrogen, sulfur, phosphorus, silicon, or combinations thereof. The heteroatom(s) may be placed at any interior position of the heteroalkyl group. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH, —S(O)—CH₃, —CH₂—CH₂—S(O)₂—CH₃, —CH₂—NH—OCH₃, —CH₂—O—Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃. Excluding the number of heteroatoms, a “heteroalkyl” may have from 1 to 6 carbon atoms.

The term “haloC₁₋₄alkoxy” or “haloC₁₋₆alkoxy” as used herein as a group or part of a group refers to a —O—C₁₋₄alkyl group or a —O—C₁₋₆ alkyl group as defined herein wherein one or more than one hydrogen atom is replaced with a halogen. The terms “haloC₁₋₄alkoxy” or “haloC₁₋₆alkoxy” therefore include monohaloC₁₋₄alkoxy, monohaloC₁₋₆alkoxy and also polyhaloC₁₋₄alkoxy and polyhaloC₁₋₆alkoxy. There may be one, two, three or more hydrogen atoms replaced with a halogen, so the haloC₁₋₄alkoxy or haloC₁₋₆alkoxy may have one, two, three or more halogens. Examples of such groups include fluoroethyloxy, difluoromethoxy, or trifluoromethoxy and the like.

The terms “fluoroalkyl” and “fluoroalkoxy” include alkyl and alkoxy groups, respectively, that are substituted with one or more fluorine atoms. Non-limiting examples of fluoroalkyls include —CF₃, —CHF₂, —CH₂F, —CH₂CF₃, —CF₂CF₃, —CF₂CF₂CF₃, —CF(CH₃)₃, and the like. Non-limiting examples of fluoroalkoxy groups, include —OCF₃, —OCHF₂, —OCH₂F, —OCH₂CF₃, —OCF₂CF₃, —OCF₂CF₂CF₃, —OCF(CH₃)₂, and the like.

The term “cyanoC₁₋₄alkyl” or “cyanoC₁₋₆alkyl” as used herein refers to a C₁₋₄alkyl or C₁₋₆alkyl group as defined herein which is substituted with one or two cyano groups, in particular with one cyano group.

“Amino” refers to a —NH₂ group.

The term “alkylamine” or “alkylamino” refers to the —N(alkyl)ₓH_{y}group, where alkyl is as defined herein and x and y are selected from the group x=1, y=1 and x=2, y=0. When x=2, the alkyl groups, taken together with the nitrogen to which they are attached, can optionally form a cyclic ring system. “Dialkylamino” refers to a —N(alkyl)₂group, where alkyl is as defined herein.

The terms “carboxy” or “carboxyl” refer to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to

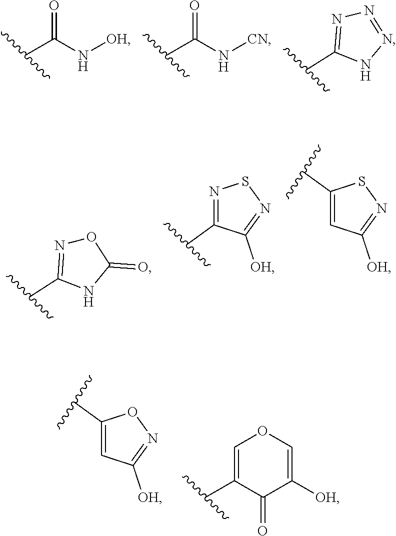

and the like.

The term “carbocyclyl” as used herein, unless the context indicates otherwise, includes aromatic, non-aromatic, unsaturated, partially saturated, and fully saturated carbon ring systems. In general, unless the context indicates otherwise, such ring systems may be monocyclic or bicyclic or bridged and may contain, for example, 3 to 12 ring members, or 4 to 10 ring members, or more usually 5 to 10 ring members. Reference to 3 to 6 ring members include 3, 4, 5, or 6 atoms in the ring, reference to 4 to 7 ring members include 4, 5, 6 or 7 atoms in the ring, and reference to 4 to 6 ring members include 4, 5, or 6 atoms in the ring. Examples of monocyclic carbocyclyl ring systems are ring systems containing 3, 4, 5, 6, 7 and 8 ring members, more usually 3 to 7, and preferably 4, 5, 6 or 7 ring members, more preferably 5 or 6 ring members. Examples of bicyclic carbocyclyl ring systems are those containing 8, 9, 10, 11 and 12 ring members, and more usually 9 or 10 ring members. Where reference is made herein to a carbocyclyl ring system, the carbocyclyl ring can, unless the context indicates otherwise, be optionally substituted (i.e. unsubstituted or substituted) by one or more substituents as discussed herein. Particular examples of 3 to 12 membered carbocycles include cyclopropyl, cyclobutyl, cyclopentyl, cyclyhexyl, cycloheptyl, cyclooctyl, phenyl naphthyl, indenyl, tetrahydronaphthyl, azulenyl, norbornane (1,4-endo-methylene-cyclohexane), adamantane ring systems.

The term “aromatic” refers to a planar ring having a delocalized π-electron system containing 4n+2 π electrons, where n is an integer. Aromatic rings can be formed from five, six, seven, eight, nine, or more than nine atoms. Aromatics can be optionally substituted. The term “aromatic” includes both aryl groups (e.g., phenyl, naphthalenyl) and heteroaryl groups (e.g., pyridinyl, quinolinyl).

The term “non-aromatic group” embraces, unless the context indicates otherwise, unsaturated ring systems without aromatic character, partially saturated and fully saturated heterocyclyl ring systems.

The terms “unsaturated” and “partially saturated” refer to rings wherein the ring structure(s) contains atoms sharing more than one valence bond i.e. the ring contains at least one multiple bond e.g. a C═C, C≡C or N═C bond.

The term “fully saturated” refers to rings where there are no multiple bonds between ring atoms. Saturated heterocyclyl groups include piperidine, morpholine, thiomorpholine, piperazine. Partially saturated heterocyclyl groups include pyrazolines, for example 2-pyrazoline and 3-pyrazoline.

The carbocyclyl ring systems can be aryl ring systems.

The term “aryl” as used herein refers to carbocyclyl aromatic groups and embraces polycyclic (e.g. bicyclic) ring systems wherein one or more rings are non-aromatic, provided that at least one ring is aromatic. In such polycyclic systems, the ring system may be attached to the remainder of the compound by an aromatic ring or by a non-aromatic ring. The term “aryl” includes phenyl, naphthyl or naphthalenyl, indenyl, and tetrahydronaphthyl groups. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. An example of a “cycloalkyl” is “C₃₋₆cycloalkyl”. Cycloalkyls may be fused with an aromatic ring (in which case the cycloalkyl is bonded through a non-aromatic ring carbon atom). Cycloalkyl groups include groups having from 3 to 10 ring atoms. Illustrative examples of cycloalkyl groups include, but are not limited to, the following moieties:

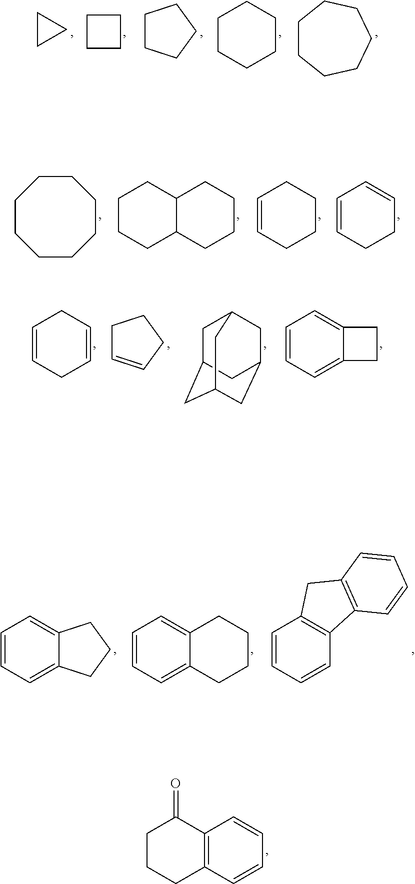

and the like.

The term “heterocyclyl”, “heterocycloalkyl”, or “heteroalicyclic” group refers to a carbocyclyl, as defined herein, containing at least one heteroatom typically selected from nitrogen, oxygen or sulphur, in particular containing up to 5, up to 4, up to 3, up to 2, or a single heteroatom. Where reference is made herein to a heterocyclyl ring system, the heterocyclyl ring can, unless the context indicates otherwise, be optionally substituted (i.e. unsubstituted or substituted) by one or more substituents as discussed herein. The radicals may be fused with an aryl or heteroaryl. Illustrative examples of heterocycloalkyl groups, also referred to as non-aromatic heterocycles, include:

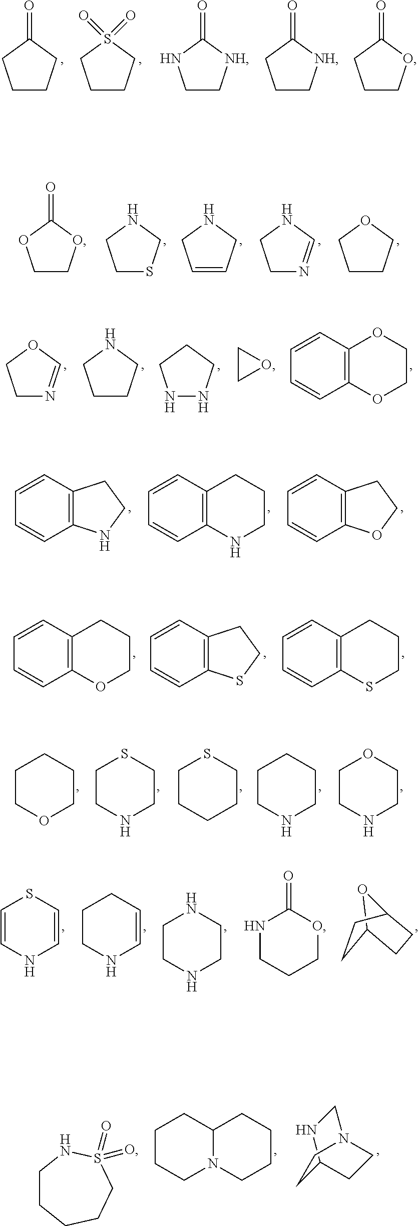

and the like.

The term heteroalicyclic also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring).

The heterocyclyl ring systems can be heteroaryl ring systems having from 5 to 12 ring members, more usually from 5 to 10 ring members.

The term “heteroaryl” is used herein to denote a heterocyclyl ring system having aromatic character. The term “heteroaryl” embraces polycyclic (e.g. bicyclic) ring systems wherein one or more rings are non-aromatic, provided that at least one ring is aromatic. In such polycyclic systems, the ring system may be attached to the remainder of the compound by an aromatic ring or by a non-aromatic ring.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings, or two fused five membered rings. The heteroaryl ring system may contain up to about five heteroatoms typically selected from nitrogen, oxygen and sulphur. Typically, the heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of five membered heteroaryl groups include but are not limited to pyrrolyl, furanyl, thienyl, imidazolyl, oxazolyl, oxadiazolyl, oxatriazole, isoxazolyl, thiazolyl, thiadiazolyl, isothiazolyl, pyrazolyl, triazolyl and tetrazolyl groups. In particular, examples of five membered heteroaryl groups include but are not limited to pyrrolyl, furanyl, thienyl, imidazolyl, oxazolyl, oxadiazolyl, isoxazolyl, thiazolyl, thiadiazolyl, isothiazolyl, pyrazolyl and triazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from: a benzene ring fused to a 5- or 6-membered ring containing 1, 2 or 3 ring heteroatoms; a pyridine ring fused to a 5- or 6-membered ring containing 0, 1, 2 or 3 ring heteroatoms; a pyrimidine ring fused to a 5- or 6-membered ring containing 0, 1 or 2 ring heteroatoms; a pyrrole ring fused to a 5- or 6-membered ring containing 0, 1, 2 or 3 ring heteroatoms; a pyrazole ring fused to a 5- or 6-membered ring containing 0, 1 or 2 ring heteroatoms; an imidazole ring fused to a 5- or 6-membered ring containing 0, 1 or 2 ring heteroatoms; an oxazole ring fused to a 5- or 6-membered ring containing 0, 1 or 2 ring heteroatoms; an isoxazole ring fused to a 5- or 6-membered ring containing 0, 1 or 2 ring heteroatoms; a thiazole ring fused to a 5- or 6-membered ring containing 0, 1 or 2 ring heteroatoms; an isothiazole ring fused to a 5- or 6-membered ring containing 0, 1 or 2 ring heteroatoms; a thiophene ring fused to a 5- or 6-membered ring containing 0, 1, 2 or 3 ring heteroatoms; a furan ring fused to a 5- or 6-membered ring containing 0, 1, 2 or 3 ring heteroatoms; a cyclohexyl ring fused to a 5- or 6-membered aromatic ring containing 1, 2 or 3 ring heteroatoms; and a cyclopentyl ring fused to a 5- or 6-membered aromatic ring containing 1, 2 or 3 ring heteroatoms.

Particular examples of bicyclic heteroaryl groups containing a five membered ring fused to another five membered ring include but are not limited to imidazothiazolyl (e.g. imidazo[2,1-b]thiazole) and imidazoimidazolyl (e.g. imidazo[1,2-a]imidazole). Particular examples of bicyclic heteroaryl groups containing a six membered ring fused to a five membered ring include but are not limited to benzofuranyl, benzothiophenyl, benzimidazolyl, benzoxazolyl, isobenzoxazolyl, benzisoxazolyl, benzthiazolyl, benzisothiazolyl, isobenzofuranyl, indolyl, isoindolyl, indolizinyl, indolinyl, isoindolinyl, purinyl, indazolyl, pyrazolopyrimidinyl (e.g. pyrazolo[1,5-a]pyrimidine), triazolopyrimidinyl (e.g. [1,2,4]triazolo[1,5-a]pyrimidine), benzodioxolyl, imidazopyrazinyl, imidazopyridazinyl, imidazopyridinyl and pyrazolopyridinyl (e.g. pyrazolo[1,5-a]pyridine) groups.

Particular examples of bicyclic heteroaryl groups containing two fused six membered rings include but are not limited to quinolizinyl, quinolinyl, isoquinolinyl, cinnolinyl, chromanyl, isochromanyl, thiochromanyl, benzopyranyl, benzodioxanyl, benzoxazinyl, pyridopyridinyl, quinoxalinyl, quinazolinyl, phthalazinyl, naphthyridinyl and pteridinyl groups.

Particular examples of bicyclic heteroaryl groups containing two fused six membered rings include but are not limited to quinolizinyl, quinolinyl, isoquinolinyl, benzopyranyl, benzodioxanyl, benzoxazinyl, pyridopyridinyl, quinoxalinyl, quinazolinyl, phthalazinyl, naphthyridinyl, and pteridinyl groups.

Examples of polycyclic heteroaryl groups containing an aromatic ring and a non-aromatic ring include, tetrahydroisoquinolinyl, tetrahydroquinolinyl, dihydrobenzothienyl, dihydrobenzofuranyl, 2,3-dihydro-benzo[1,4]dioxinyl, benzo[1,3]dioxolyl, 4,5,6,7-tetrahydrobenzofuranyl, tetrahydrotriazolopyrazinyl (e.g. 5,6,7,8-tetrahydro-[1,2,4]triazolo[4,3-a]pyrazinyl), and indolinyl.

A nitrogen-containing heteroaryl ring must contain at least one ring nitrogen atom.

Each ring may, in addition, contain up to about four other heteroatoms typically selected from nitrogen, sulphur and oxygen. Typically, the heteroaryl ring will contain up to 3 heteroatoms, for example 1, 2 or 3, more usually up to 2 nitrogens, for example a single nitrogen. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of nitrogen-containing heteroaryl groups include, but are not limited to, pyridyl, pyrrolyl, imidazolyl, oxazolyl, oxadiazolyl, thiadiazolyl, oxatriazolyl, isoxazolyl, thiazolyl, isothiazolyl, pyrazolyl, pyrazinyl, pyrimidinyl, pyridazinyl, triazinyl, triazolyl (e.g., 1,2,3-triazolyl, 1,2,4-triazolyl), tetrazolyl, quinolinyl, isoquinolinyl, benzimidazolyl, benzoxazolyl, benzisoxazolyl, benzthiazolyl and benzisothiazole, indolyl, 3H-indolyl, isoindolyl, indolizinyl, isoindolinyl, purinyl, indazolyl, quinolizinyl, benzoxazinyl, pyridopyridinyl, quinoxalinyl, quinazolinyl, cinnolinyl, phthalazinyl, naphthyridinyl, and pteridinyl.

Examples of nitrogen-containing polycyclic heteroaryl groups containing an aromatic ring and a non-aromatic ring include tetrahydroisoquinolinyl, tetrahydroquinolinyl, and indolinyl.

Examples of non-aromatic heterocyclyl groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur. The heterocyclyl groups can contain, for example, cyclic ether moieties (e.g. as in tetrahydrofuran and dioxane), cyclic thioether moieties (e.g. as in tetrahydrothiophene and dithiane), cyclic amine moieties (e.g. as in pyrrolidine), and combinations thereof (e.g. thiomorpholine).

Particular examples include morpholinyl, thiomorpholinyl, piperidinyl (e.g. 1-piperidinyl, 2-piperidinyl, 3-piperidinyl and 4-piperidinyl), pyrrolidinyl (e.g. 1-pyrrolidinyl, 2-pyrrolidinyl and 3-pyrrolidinyl), azetidinyl, pyranyl (2H-pyranyl or 4H-pyranyl), dihydrothiophenyl, dihydropyranyl, dihydrofuranyl, dihydrothiazolyl, tetrahydrofuranyl, tetrahydrothiophenyl, dioxanyl, dioxolanyl, tetrahydropyranyl, imidazolinyl, oxazolinyl, oxazolidinyl, oxetanyl, thiazolinyl, 2-pyrazolinyl, pyrazolidinyl and piperazinyl. In general, preferred non-aromatic heterocyclyl groups include saturated groups such as piperidinyl, pyrrolidinyl, azetidinyl, morpholinyl and piperazinyl. In general, preferred non-aromatic heterocyclyl groups include saturated groups such as piperidinyl, pyrrolidinyl, azetidinyl, morpholinyl and piperazinyl.

In a nitrogen-containing non-aromatic heterocyclyl ring the ring must contain at least one ring nitrogen atom.

Particular examples of nitrogen-containing non-aromatic heterocyclyl groups include aziridinyl, morpholinyl, thiomorpholinyl, piperidinyl (e.g. 1-piperidinyl, 2-piperidinyl, 3-piperidinyl and 4-piperidinyl), pyrrolidinyl (e.g. 1-pyrrolidinyl, 2-pyrrolidinyl and 3-pyrrolidinyl), dihydrothiazolyl, imidazolinyl, oxazolinyl, thiazolinyl, 2-pyrazolinyl, 3-pyrazolinyl, pyrazolidinyl and piperazinyl.

Particular examples of 3 to 6 membered monocyclic saturated heterocyclyls include morpholinyl, thiomorpholinyl, dioxanyl, piperidinyl (e.g. 1-piperidinyl, 2-piperidinyl, 3-piperidinyl and 4-piperidinyl), piperazinyl, pyrrolidinyl (e.g. 1-pyrrolidinyl, 2-pyrrolidinyl and 3-pyrrolidinyl), imidazolidinyl, pyrazolidinyl, oxazolidinyl, isoxazolidinyl, thiazolidinyl, isothiazolidinyl, dioxolanyl, dithiolanyl, tetrahydrofuranyl, tetrahydrothiophenyl, tetrahydropyranyl (e.g. 4-tetrahydro pyranyl), dithianyl, trioxanyl, trithianyl, aziridinyl, oxiranyl, thiiranyl, diaziridinyl, dioxarinyl, oxetanyl, azetidinyl, thietanyl, dioxetanyl ring systems.

Particular examples of 3 to 6 membered monocyclic heterocyclyls include morpholinyl, thiomorpholinyl, piperidinyl (e.g. 1-piperidinyl, 2-piperidinyl, 3-piperidinyl and 4-piperidinyl), pyrrolidinyl (e.g. 1-pyrrolidinyl, 2-pyrrolidinyl and 3-pyrrolidinyl), imidazolidinyl, pyrazolidinyl, oxazolidinyl, isoxazolidinyl, thiazolidinyl, isothiazolidinyl, dioxolanyl, dithiolanyl, piperazinyl, tetrahydrofuranyl, tetrahydrothiophenyl, dioxanyl, tetrahydropyranyl (e.g. 4-tetrahydro pyranyl), dithianyl, trioxanyl, trithianyl, aziridinyl, oxiranyl, thiiranyl, diaziridinyl, dioxarinyl, oxetanyl, azetidinyl, thietanyl, dioxetanyl, azirinyl, azetyl, 1,2-dithietyl, pyrrolyl, furanyl, thiophenyl, imidazolyl, pyrazolyl, oxazolyl, thiazolyl, isothiazolyl, triazolyl, oxadiazolyl, thiadiazolyl, dithiazolyl, pyridinyl, pyranyl, thiopyranyl, pyrimidinyl, thiazinyl, oxazinyl, triazinyl ring systems.

Particular examples of 3 to 12 membered heterocycles include morpholinyl, thiomorpholinyl, piperidinyl (e.g. 1-piperidinyl, 2-piperidinyl, 3-piperidinyl and 4-piperidinyl), pyrrolidinyl (e.g. 1-pyrrolidinyl, 2-pyrrolidinyl and 3-pyrrolidinyl), imidazolidinyl, pyrazolidinyl, oxazolidinyl, isoxazolidinyl, thiazolidinyl, isothiazolidinyl, dioxolanyl, dithiolanyl, piperazinyl, tetrahydrofuranyl, tetrahydrothiophenyl, dioxanyl, tetrahydropyranyl (e.g. 4-tetrahydropyranyl), dithianyl, trioxanyl, trithianyl, aziridinyl, oxiranyl, thiiranyl, diaziridinyl, dioxarinyl, oxetanyl, azetidinyl, thietanyl, dioxetanyl, azirinyl, azetyl, 1,2-dithietyl, pyrrolyl, furanyl, thiophenyl, imidazolyl, pyrazolyl, oxazolyl, thiazolyl, isothiazolyl, triazolyl, oxadiazolyl, thiadiazolyl, dithiazolyl, pyridinyl, pyranyl, thiopyranyl, pyrimidinyl, thiazinyl, oxazinyl, triazinyl, azepanyl, oxepanyl, thiepanyl, 1,2-diazepanyl, 1,4-diazepanyl, diazepinyl, thiazepinyl, azocanyl, azocinyl, imidazothiazolyl (e.g. imidazo[2,1-b]thiazolyl), imidazo-imidazolyl (e.g. imidazo[1,2-a]imidazolyl), benzofuranyl, benzothiophenyl, benzimidazolyl, benzoxazolyl, isobenzoxazolyl, benzisoxazolyl, benzthiazolyl, benzisothiazolyl, isobenzofuranyl, indolyl, isoindolyl, indolizinyl, indolinyl, isoindolinyl, purinyl, indazolyl, pyrazolopyrimidinyl (e.g. pyrazolo[1,5-a]pyrimidinyl), triazolopyrimidinyl (e.g. [1,2,4]triazolo[1,5-a]pyrimidinyl), benzodioxolyl, imidazopyridinyl and pyrazolopyridinyl (e.g. pyrazolo[1,5-a]pyridinyl), quinolinyl, isoquinolinyl, chromanyl, thiochromanyl, isochromanyl, benzodioxanyl, quinolizinyl, benzoxazinyl, pyridopyridinyl, quinoxalinyl, quinazolinyl, cinnolinyl, phthalazinyl, naphthyridinyl, pteridinyl, tetrahydroisoquinolinyl, tetrahydroquinolinyl, dihydrobenzthienyl, dihydrobenzfuranyl, 2,3-dihydro-benzo[1,4]dioxinyl, benzo[1,3]dioxolyl, 4,5,6,7-tetrahydrobenzofuranyl, tetrahydrotriazolopyrazinyl (e.g. 5,6,7,8-tetrahydro-[1,2,4]triazolo[4,3-a]pyrazinyl), 8-oxa-3-azabicyclo[3.2.1]octanyl, 2-oxa-5-azabicyclo[2.2.1]heptanyl, 3-oxa-8-azabicyclo[3.2.1]octanyl, 3,6-diazabicyclo[3.1.1]heptanyl ring systems.

Particular examples of 5 to 6 membered aromatic heterocycles include but are not limited to pyrrolyl, furanyl, thiophenyl, imidazolyl, furazanyl, oxazolyl, oxadiazolyl, oxatriazolyl, isoxazolyl, thiazolyl, thiadiazolyl, isothiazolyl, pyrazolyl, triazolyl, tetrazolyl, pyridinyl, pyrazinyl, pyridazinyl, pyrimidinyl and triazinyl ring systems.

The heterocyclyl and carbocyclyl rings also include bridged ring systems such as for example bridged cycloalkanes, such as for example norbornane (1,4-endo-methylene-cyclohexane), adamantane, oxa-adamantane; bridged morpholine rings such as for example 8-oxa-3-azabicyclo[3.2.1]octane, 2-oxa-5-azabicyclo[2.2.1]heptane, 3-oxa-8-azabicyclo[3.2.1]octane; bridged piperazine rings such as for example 3,6-diazabicyclo[3.1.1]heptane; bridged piperidine rings such as for example 1,4-ethylenepiperidine. For an explanation of the distinction between fused and bridged ring systems, see Advanced Organic Chemistry, by Jerry March, 4^{th} Edition, Wiley Interscience, pages 131-133, 1992.

Lines drawn into ring systems indicate that the bond may be attached to any of the suitable and available ring atoms.

The term “optional” or “optionally” means the event described subsequent thereto may or may not happen. This term encompasses the cases that the event may or may not happen.

In the compounds of the present disclosure the carbon atom indicated with a “*” in the drawn formula, is a chiral center. When the carbon atom is indicated with “(R*)”, it means that it is a pure enantiomer but that it is unknown whether is it an R or S enantiomer. Similarly, when the carbon atom is indicated with “(S*)”, it means that it is a pure enantiomer but that it is unknown whether is it an R or S enantiomer.

The term “bond” or “single bond” refers to a chemical bond between two atoms, or two moieties when the atoms joined by the bond are considered to be part of larger substructure.

The term “moiety” refers to a specific segment or functional group of a molecule.

Chemical moieties are often recognized chemical entities embedded in or appended to a molecule.

As used herein, the substituent “R” appearing by itself and without a number designation refers to a substituent selected from among from alkyl, haloalkyl, heteroalkyl, alkenyl, cycloalkyl, aryl, heteroaryl (bonded through a ring carbon), and heterocycloalkyl.

The term “optionally substituted” or “substituted”, if not explicitly defined, means that the referenced group may be substituted with one or more additional group(s) individually and independently selected from alkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkynyl, C₁₋₆alkylalkynyl, halo, acyl, acyloxy, —CO₂H, —CO₂-alkyl, nitro, haloalkyl, fluoroalkyl, and amino, including mono- and di-substituted amino groups (e.g. —NH₂, —NHR, —N(R)₂), and the protected derivatives thereof. In some embodiments, optional substituents are independently selected from halogen, —CN, —NH₂, —NH(CH₃), —N(CH₃)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

The term a “therapeutically effective amount” as used herein refers to the amount of active compound or pharmaceutical agent that, when administered to a mammal in need, is effective to at least partially ameliorate or to at least partially prevent diseases, disorders or conditions described herein.

As used herein, the term “composition” is intended to encompass a product comprising specified ingredients in specified amounts, as well as any product which results, directly or indirectly, from combinations of the specified ingredients in the specified amounts.

As used herein, the term “expression” includes the process by which polynucleotides are transcribed into mRNA and translated into peptides, polypeptides, or proteins. The term “activator” is used in this specification to denote any molecular species that results in activation of the indicated receptor, regardless of whether the species itself binds to the receptor or a metabolite of the species binds to the receptor. Thus, the activator can be a ligand of the receptor or it can be an activator that is metabolized to the ligand of the receptor, i.e., a metabolite that is formed in tissue and is the actual ligand.

The term “antagonist” as used herein, refers to a small-molecule agent that binds to a receptor and subsequently decreases the agonist induced transcriptional activity of the receptor.

The term “agonist” as used herein, refers to a small-molecule agent that binds to a receptor and subsequently increases receptor transcriptional activity in the absence of a known agonist.

The term “inverse agonist” as used herein, refers to a small-molecule agent that binds to a receptor and subsequently decreases the basal level of receptor transcriptional activity that is present in the absence of a known agonist.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. Those skilled in the art recognize that a therapy which reduces the severity of a pathology in one species of mammal is predictive of the effect of the therapy on another species of mammal.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

A “proliferative disease” refers to a disease that occurs due to abnormal growth or extension by the multiplication of cells. A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue.

Furthermore, a malignant neoplasm generally has the capacity to metastasize to distant sites.

As used herein, the term “cancer” refers to a malignant neoplasm. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases).

As used herein, an “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis.

As used herein, an “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response.

Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid, arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme arthritis, Guillain-Barre syndrome, Hashimoto's thyroiditis, and cardiomyopathy.

The term “autoinflammatory disease” refers to a category of diseases that are similar but different from autoimmune diseases. Autoinflammatory and autoimmune diseases share common characteristics in that both groups of disorders result from the immune system attacking a subject's own tissues and result in increased inflammation. In autoinflammatory diseases, a subject's innate immune system causes inflammation for unknown reasons. The innate immune system reacts even though it has never encountered autoantibodies or antigens in the subject. Autoinflammatory disorders are characterized by intense episodes of inflammation that result in such symptoms as fever, rash, or joint swelling. These diseases also carry the risk of amyloidosis, a potentially fatal buildup of a blood protein in vital organs. Autoinflammatory diseases include, but are not limited to, familial Mediterranean fever (FMF), neonatal onset multisystem inflammatory disease (NOMID), tumor necrosis factor (TNF) receptor-associated periodic syndrome (TRAPS), deficiency of the interleukin-1 receptor antagonist (DIRA), and Behget's disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as transgenic oocyte, sperm cell, blastocyst, embryo, fetus, donor cell, or cell nucleus.

### Isomers, Salts, N-Oxides, Solvates, Polymorphs, Prodrugs, Isotopically Labeled Derivatives

Hereinbefore and hereinafter, the term “compound of formula (I), (II), (IIIa), (IIb), (IVa), (IVb), (Va), (Vb)”, “compounds of the present disclosure or invention”, “compounds presented herein”, or similar terms, is meant to include the addition salts, the solvates and the stereoisomers thereof.

In certain embodiments, the compounds presented herein possess one or more stereocenters and each center independently exists in either the R or S configuration.

The compounds presented herein include all diastereomeric, enantiomeric, atropisomers, and epimeric forms as well as the appropriate mixtures thereof.

Stereoisomers are obtained, if desired, by methods such as, stereoselective synthesis and/or the separation of stereoisomers by chiral chromatographic columns. In some embodiments, a compound of the present disclosure is used as a single enantiomer. In some embodiments, a compound of the present disclosure is used as a racemic mixture. In some embodiments, a compound of the present disclosure possesses hindered rotation about a single bond resulting in atropisomers.

In some situations, compounds may exist as tautomers. All tautomers are included within the scope of the compounds presented herein.

For the avoidance of doubt, where a compound can exist in one of several geometric isomeric or tautomeric forms and only one is specifically described or shown, all others are nevertheless embraced. Examples of tautomeric forms include, for example, keto-, enol-, and enolate-forms, as in, for example, the following tautomeric pairs: keto/enol (illustrated below), imine/enamine, amide/imino alcohol, amidine/enediamines, nitroso/oxime, thioketone/enethiol, and nitro/aci-nitro.

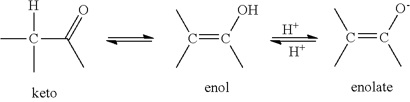

Such forms in so far as they may exist, are intended to be included within the scope of the compounds presented herein. It follows that a single compound may exist in both stereoisomeric and tautomeric form.

Where compounds described herein contain one or more chiral centres, and can exist in the form of two or more optical isomers, references to the compounds described herein include all optical isomeric forms thereof (e.g. enantiomers, epimers and diastereoisomers), either as individual optical isomers, or mixtures (e.g. racemic mixtures) of two or more optical isomers, unless the context requires otherwise. When a compound has more than one chiral centre, and one chiral centre is indicated as having an absolute stereoconfiguration, the other chiral centre(s) include all optical isomeric forms, either as individual optical isomers, or mixtures (e.g. racemic mixtures) of two or more optical isomers, thereof, unless the context requires otherwise. The optical isomers may be characterized and identified by their optical activity (i.e. as + and − isomers depending on the direction in which they rotate plane polarized light, or d and l isomers) or they may be characterized in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog (1966)Angew. Chem. Int. Ed. Engl., 5, 385-415. For instance, resolved enantiomers whose absolute configuration is not known can be designated by (+) or (−) depending on the direction in which they rotate plane polarized light.

Optical isomers can be separated by a number of techniques including chiral chromatography (chromatography on a chiral support) and such techniques are well known to the person skilled in the art. As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds exist as two or more isomeric forms, one isomeric form, e.g. one enantiomer in a pair of enantiomers, may exhibit advantages over the other isomeric form, e.g. over the other enantiomer, for example, in terms of biological activity. Thus, in certain circumstances, it may be desirable to use as a therapeutic agent only one of a pair of enantiomers, or only one of a plurality of diastereoisomers.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound described herein is for instance specified as (S), this means that the compound is substantially free of the (R) isomer; when a compound described herein is for instance specified as E, this means that the compound is substantially free of the Z isomer; when a compound described herein is for instance specified as cis, this means that the compound is substantially free of the trans isomer.

As used herein, any chemical formula with bonds shown only as solid lines and not as solid wedged or hashed wedged bonds, or otherwise not indicated as having a particular configuration (e.g. R, S) around one or more atoms, contemplates each possible stereoisomer, or mixture of two or more stereoisomers.

The terms “stereoisomers”, “stereoisomeric forms” or “stereochemically isomeric forms” hereinbefore or hereinafter are used interchangeably.

Enantiomers are stereoisomers that are non-superimposable mirror images of each other. A 1:1 mixture of a pair of enantiomers is a racemate or racemic mixture. Atropisomers (or atropoisomers) are stereoisomers which have a particular spatial configuration, resulting from a restricted rotation about a single bond, due to large steric hindrance. All atropisomeric forms of the compounds described herein are intended to be included within the scope of the present invention.

Diastereomers (or diastereoisomers) are stereoisomers that are not enantiomers, i.e. they are not related as mirror images. If a compound contains a double bond, the substituents may be in the E or the Z configuration. Substituents on bivalent cyclic (partially) saturated radicals may have either the cis- or trans-configuration; for example if a compound contains a disubstituted cycloalkyl group, the substituents may be in the cis or trans configuration. Therefore, the present disclosure includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The methods and formulations described herein include the use of N-oxides (if appropriate), crystalline forms (also known as polymorphs), solvates and hydrates (also known as pseudo-polymorphs), pharmaceutically acceptable salts, and combinations thereof, of compounds having the structures presented herein, as well as active metabolites of these compounds having the same type of activity.

In some embodiments, compounds described herein, are in various forms, including but not limited to, amorphous forms, milled forms and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, melting points, density, hardness, crystal shape, optical properties, stability, and solubility. Various factors such as the recrystallization solvent, rate of crystallization, and storage temperature may cause a single crystal form to dominate. In specific embodiments, the compounds described herein exist in solvated forms with pharmaceutically acceptable solvents such as water, ethanol, and the like. In other embodiments, the compounds described herein exist in unsolvated form.

In some embodiments, the compounds described herein include solvent addition forms or crystal forms thereof, particularly solvates or polymorphs. As used herein, the term “solvate” means a physical association of the compounds of the present invention with one or more solvent molecules, as well as pharmaceutically acceptable addition salts thereof. This physical association involves varying degrees of ionic and covalent bonding, including hydrogen bonding. In certain instances, the solvate will be capable of isolation, for example when one or more solvent molecules are incorporated in the crystal lattice of the crystalline solid. The term “solvate” is intended to encompass both solution-phase and isolatable solvates. Solvates contain either stoichiometric or non-stoichiometric amounts of a solvent, and may be formed during the process of crystallization with pharmaceutically acceptable solvents such as water, ethanol, isopropanol, methanol, DMSO, ethyl acetate, acetic acid, ethanolamine and the like. Hydrates are formed when the solvent is water, or alcoholates are formed when the solvent is alcohol. The compounds described herein may exert their biological effects whilst they are in solution.

The salt forms of the compounds presented herein are typically pharmaceutically acceptable salts, and examples of pharmaceutically acceptable salts are discussed in Berge et al. (1977) “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

The pharmaceutically acceptable salts include pharmaceutically acceptable acid and base addition salts and are meant to comprise the therapeutically active non-toxic acid and base addition salt forms that the compounds described herein are able to form. The salts of the present disclosure can be synthesized from the parent compound that contains a basic or acidic moiety by conventional chemical methods such as methods described in “Pharmaceutical Salts: Properties, Selection, and Use”, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used. The compounds of the invention may exist as mono- or di-salts depending upon the pKa of the acid from which the salt is formed.

The pharmaceutically acceptable acid addition salts can conveniently be obtained by treating the base form with such appropriate inorganic acid (such as hydrochloric acid, hydrobromic acid, sulfuric acid, nitric acid, phosphoric acid and the like) or organic acids such (as acetic acid, methanesulfonic acid, maleic acid, tartaric acid, citric acid and the like) in an anion form.

Appropriate anions comprise, for example, acetate, 2,2-dichloroacetate, adipate, alginate, ascorbate (e.g. L-ascorbate), L-aspartate, benzenesulfonate, benzoate, 4-acetamidobenzoate, butanoate, bicarbonate, bitartrate, bromide, (+) camphorate, camphor-sulphonate, (+)-(1S)-camphor-10-sulphonate, calcium edetate, camsylate, caprate, caproate, caprylate, carbonate, chloride, cinnamate, citrate, cyclamate, dihydrochloride, dodecylsulphate, edetate, estolate, esylate, ethane-1,2-disulphonate, ethanesulphonate, formate, fumarate, galactarate, gentisate, glucoheptonate, gluceptate, gluconate, D-gluconate, glucuronate (e.g. D-glucuronate), glutamate (e.g. L-glutamate), α-oxoglutarate, glycolate, glycollylarsanilate, hexylresorcinate, hippurate, hydrabamine, hydrobromide, hydrochloride, hydriodate, 2-hydroxyethane-sulphonate, hydroxynaphthoate, iodide, isethionate, lactate (e.g. (+)-L-lactate, (±)-DL-lactate), lactobionate, malate, (−)-L-malate, maleate, malonate, mandelate, (±)-DL-mandelate, mesylate, methansulfonate, methylbromide, methylnitrate, methylsulfate, mucate, naphthalene-sulphonate (e.g. naphthalene-2-sulphonate), naphthalene-1,5-disulphonate, 1-hydroxy-2-naphthoate, napsylate, nicotinate, nitrate, oleate, orotate, oxalate, palmitate, pamoate (embonate), pantothenate, phosphate/diphosphate, propionate, polygalacturonate, L-pyroglutamate, pyruvate, salicylate, 4-amino-salicylate, sebacate, stearate, subacetate, succinate, sulfate, tannate, tartrate, (+)-L-tartrate, teoclate, thiocyanate, toluenesulphonate (e.g. p-toluenesulphonate), tosylate, triethiodide, undecylenate, valeric acids, as well as acylated amino acids and cation exchange resins. Conversely said salt forms can be converted by treatment with an appropriate base into the free base form.

The compounds of the present disclosure containing an acidic proton may also be converted into their nontoxic metal or amine addition salt forms by treatment with appropriate organic and inorganic bases in a cation form. Appropriate basic salts comprise those formed with organic cations such as arginine, benzathine, benzylamine, butylamine, chloroprocaine, choline, diethanolamine, dicyclohexylamine, diethanolamine, diethylamine, ethanolamine, ethylamine, ethylenediamine, lysine, meglumine, phenylbenzylamine, piperazine, procaine, triethylamine, tromethamine, and the like; those formed with ammonium ion (i.e., NH₄⁺), quaternary ammonium ion N(CH₃)₄⁺, and substituted ammonium ions (e.g., NH₃R⁺, NH₂R₂⁺, NHR₃⁺, NR₄⁺); and those formed with metallic cations such as aluminum, calcium, lithium, magnesium, potassium, sodium, zinc, and the like. Where the compounds described herein contain an amine function, these may form quatemary ammonium salts, for example by reaction with an alkylating agent according to methods well known to the skilled person. Such quaternary ammonium compounds are within the scope of the compounds presented herein.

Conversely said salt forms can be converted by treatment with an appropriate acid into the free form.

The screening and characterization of the pharmaceutically acceptable salts, polymorphs and/or solvates may be accomplished using a variety of techniques including, but not limited to, thermal analysis, X-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravimetric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). Solid State NMR (SS-NMR) is also known as Magic Angle Spinning NMR or MAS-NMR. The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quatemary derivatives of tertiary amines, N-Mannich bases, Schiff bases, amino acid conjugates, phosphate esters, and sulfonate esters. See for example Vivekkumar K. and Bari S. “Prodrug Design”, Academic Press, 2016; Rautio, J. and Laine, K. “Prodrugs in Drug Design and Development” in “Textbook of Drug Design and Development”, Strømgaard, Krogsgaard-Larsen, and Madsen, Ed. 5, 2017, Chapter 10; and Di and Kerns, “Prodrugs” in “Drug-Like Properties”, 2016, 2^{nd}, Ed. 471-485, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like.

Prodrug forms of the herein described compounds, wherein the prodrug is metabolized in vivo to produce a compound of the present disclosure, as set forth herein, are included within the scope of the claims. In some cases, some of the herein-described compounds may be a prodrug for another derivative or active compound.

In some embodiments, sites on the compounds disclosed herein are susceptible to various metabolic reactions. Therefore, incorporation of appropriate substituents at the places of metabolic reactions will reduce, minimize or eliminate the metabolic pathways. In specific embodiments, the appropriate substituent to decrease or eliminate the susceptibility of the aromatic ring to metabolic reactions is, by way of example only, a halogen, deuterium or an alkyl group.

The compounds of the present disclosure include compounds that are isotopically labeled, i.e., with one or more isotopic substitutions. These compounds are identical to those recited in the various formulae and structures presented herein, but for the fact that one or more atoms are replaced by an atom having an atomic mass or mass number different from the atomic mass or mass number usually found in nature. A reference to a particular element includes within its scope all isotopes of the element, either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. In one embodiment of the invention, the compounds contain no radioactive isotopes. In another embodiment, the compound may contain one or more radioisotopes. Compounds containing such radioisotopes may also be useful in a diagnostic context. Radiolabeled compounds described herein may comprise a radioactive isotope selected from the group of ²H, ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ²H, ³H, ¹¹C and ¹⁸F. More preferably, the radioactive isotope is ²H. In particular, deuterated compounds are intended to be included within the scope of the present invention. In some embodiments, metabolic sites on the compounds described herein are deuterated.

Throughout the specification, groups and substituents thereof can be chosen to provide stable moieties and compounds.

### Synthesis of Compounds

The synthesis of compounds described herein, particularly in the Examples section, are accomplished using means described in the chemical literature, using the methods described herein, or by a combination thereof. In addition, solvents, temperatures, and other reaction conditions presented herein may vary. Techniques and materials recognized in the field are described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified using appropriate reagents and conditions, for the introduction of the various moieties found in the formulae as provided herein.

The starting materials and reagents used for the synthesis of the compounds described herein may be synthesized or obtained from commercial sources, such as, but not limited to, Sigma-Aldrich, FischerScientific (Fischer Chemicals), and AcrosOrganics. In the reactions described herein, it may be necessary to protect reactive functional groups, for example hydroxy, amino, imino, thio or carboxy groups, where these are desired in the final product, in order to avoid their unwanted participation in reactions. Protecting groups are used to block some or all of the reactive moieties and prevent such groups from participating in chemical reactions until the protective group is removed. It is preferred that each protective group be removable by a different means. Protective groups that are cleaved under totally disparate reaction conditions fulfill the requirement of differential removal.

Protective groups can be removed by acid, base, reducing conditions (such as, for example, hydrogenolysis), and/or oxidative conditions. Groups such as trityl, dimethoxytrityl, acetal and t-butyl dimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable. Carboxylic acid and hydroxy reactive moieties may also be blocked with hydrolytically removable protective groups such as the benzyl group, while amine groups capable of hydrogen bonding with acids may be blocked with base labile groups such as acetyl, trifluoroacetyl, t-butoxycarbonyl (Boc), benzyloxycarbonyl (CBz), and 9-fluorenylmethyleneoxycarbonyl (Fmoc). Carboxylic acid reactive moieties may be protected by conversion to simple ester compounds as exemplified herein, which include conversion to alkyl esters, or they may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a Pd⁰-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

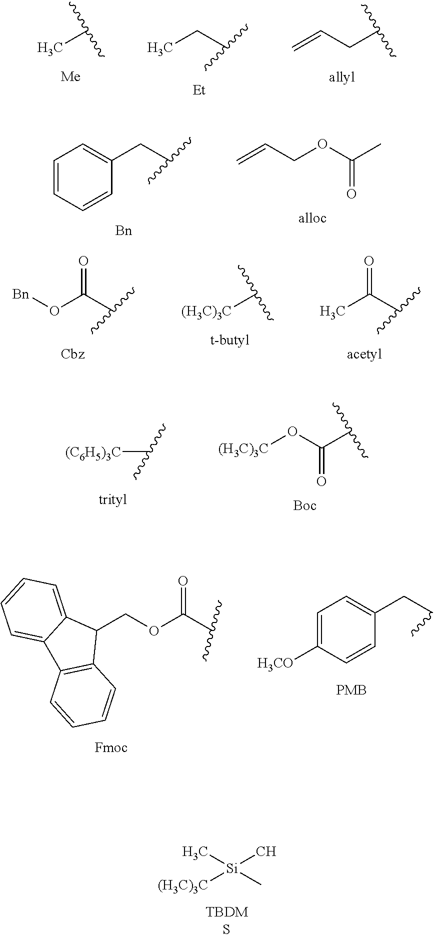

Other protecting groups, plus a detailed description of techniques applicable to the creation of protecting groups and their removal are described in T. W. Greene and P. G. M. Wuts, Protective Groups in Organic Synthesis, 4th ed., Wiley, Hoboken, New Jersey, 2007, which is incorporated herein by reference for such disclosure.

The skilled person will realize that intermediates and final compounds shown in the schemes below may be further functionalized according to methods well-known by the person skilled in the art.

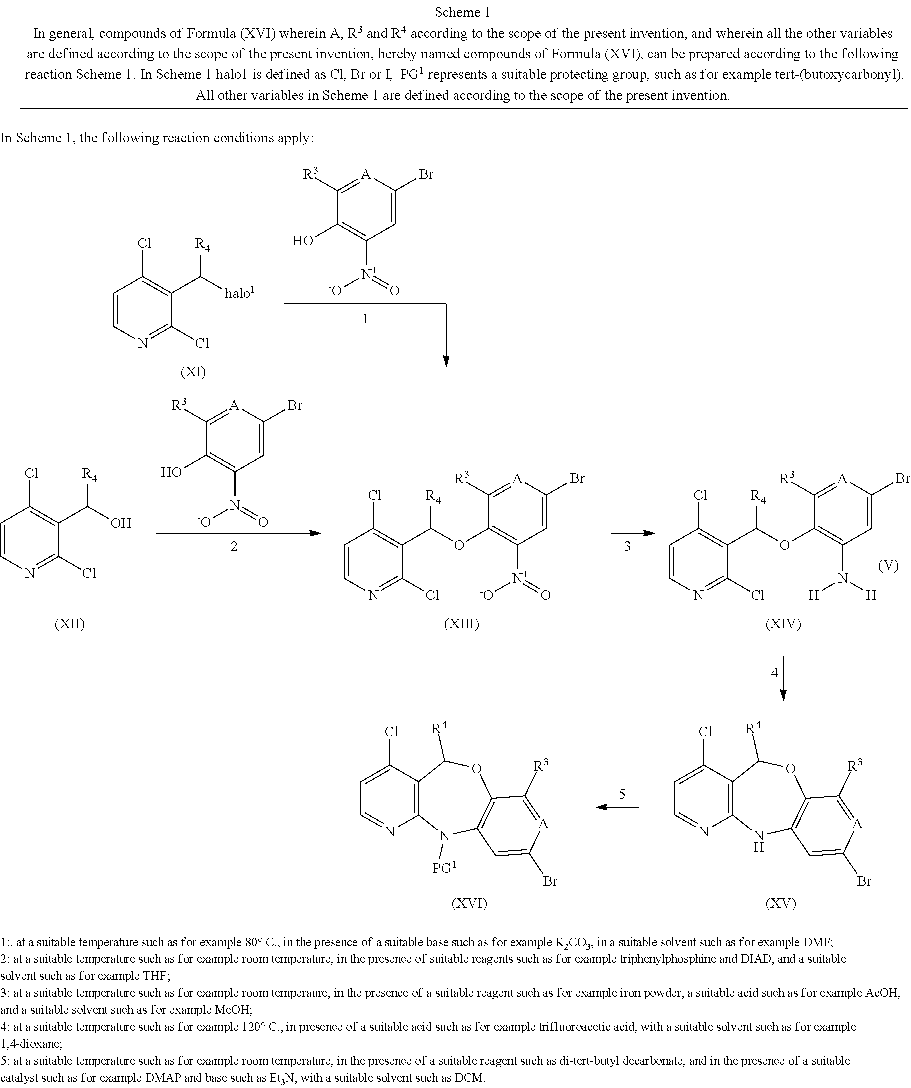

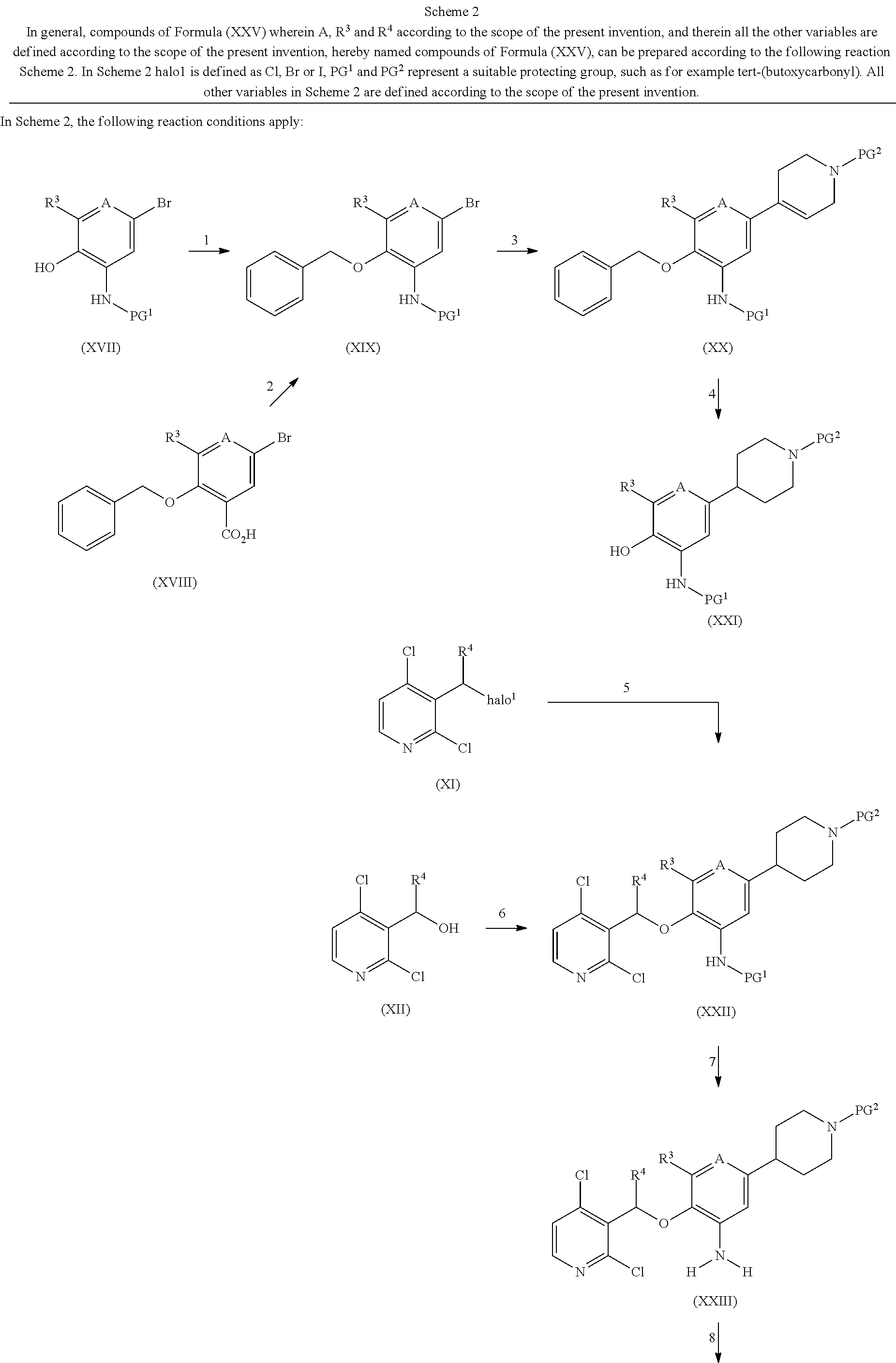

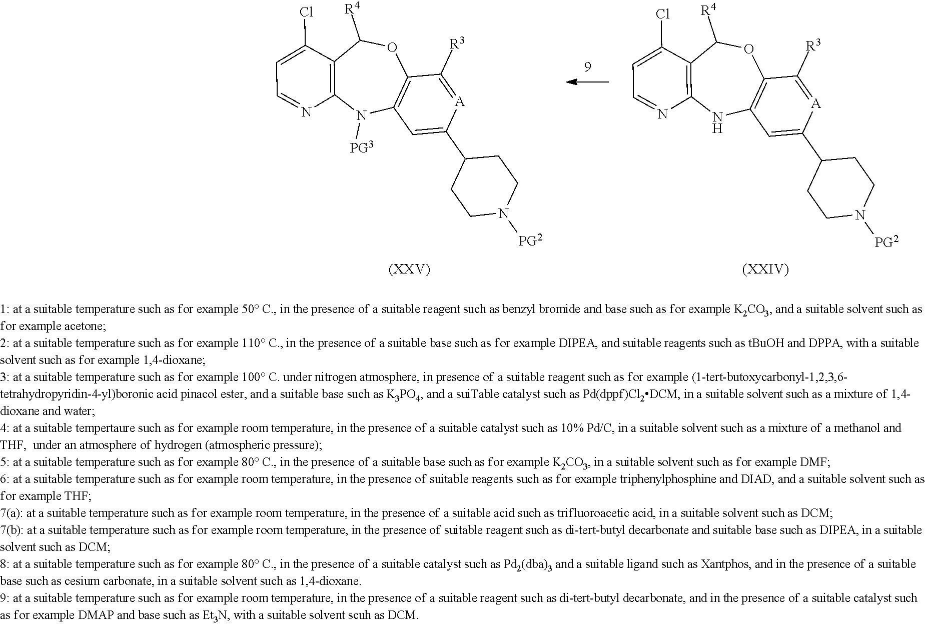

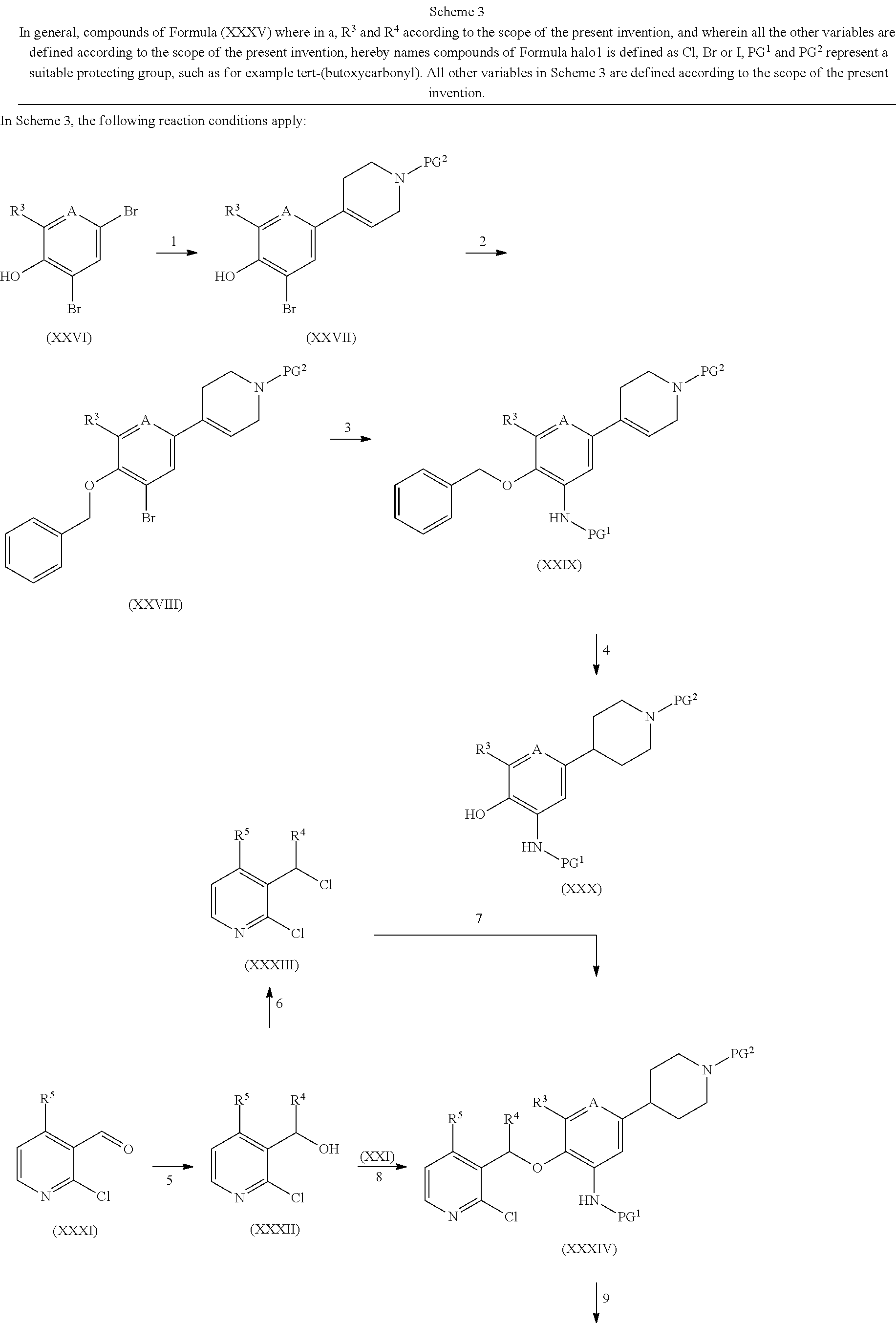

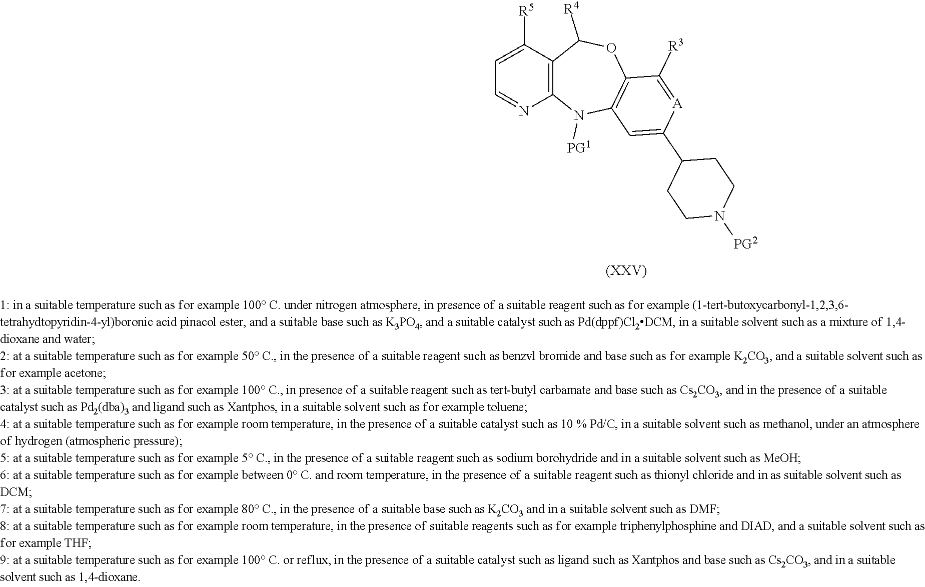

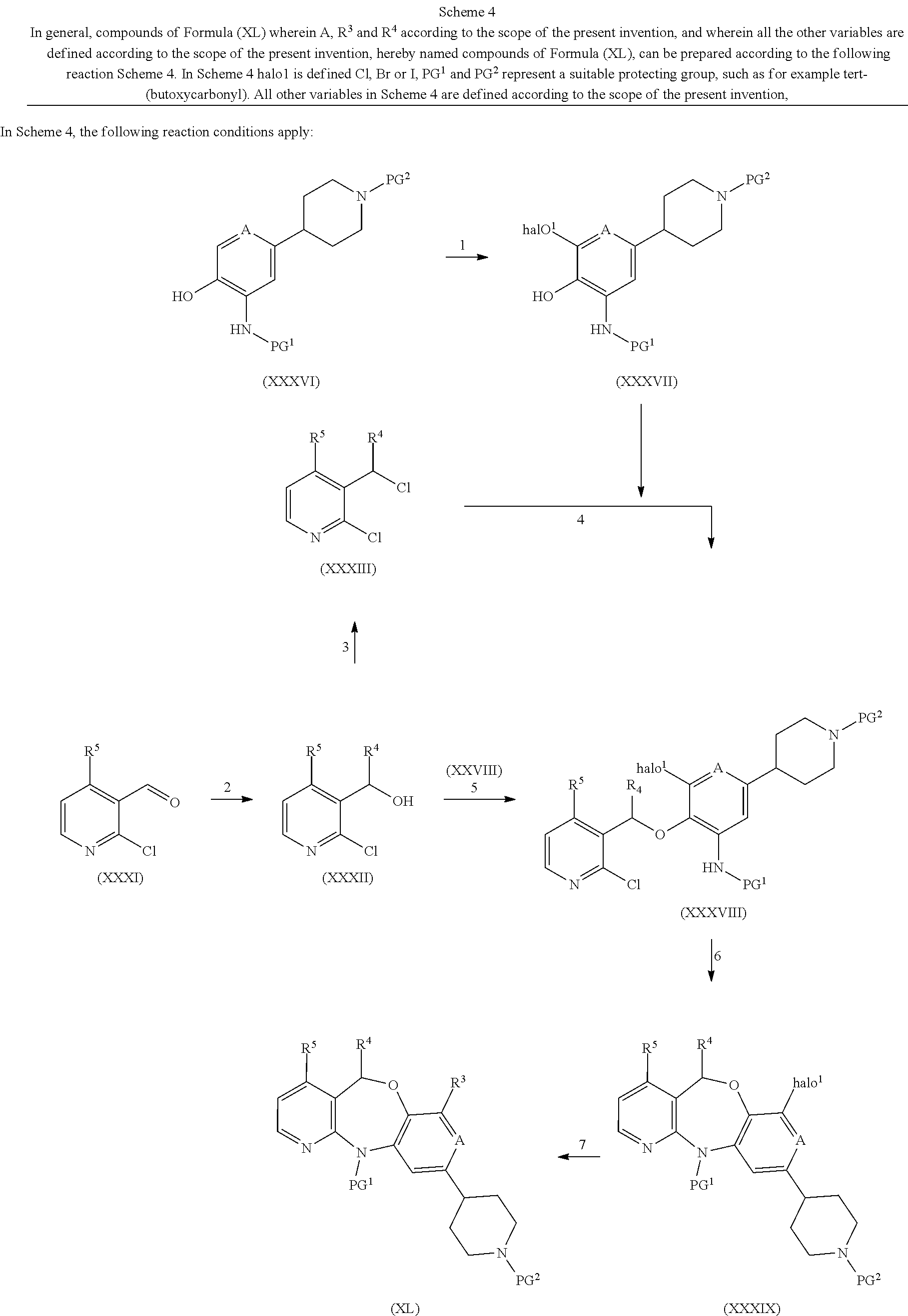

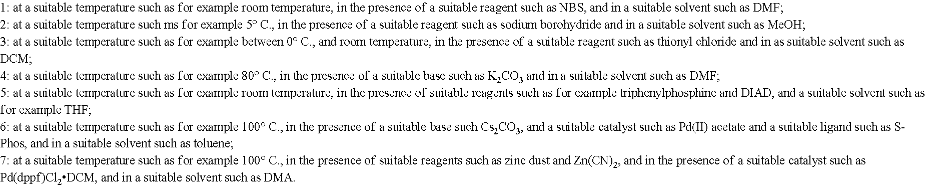

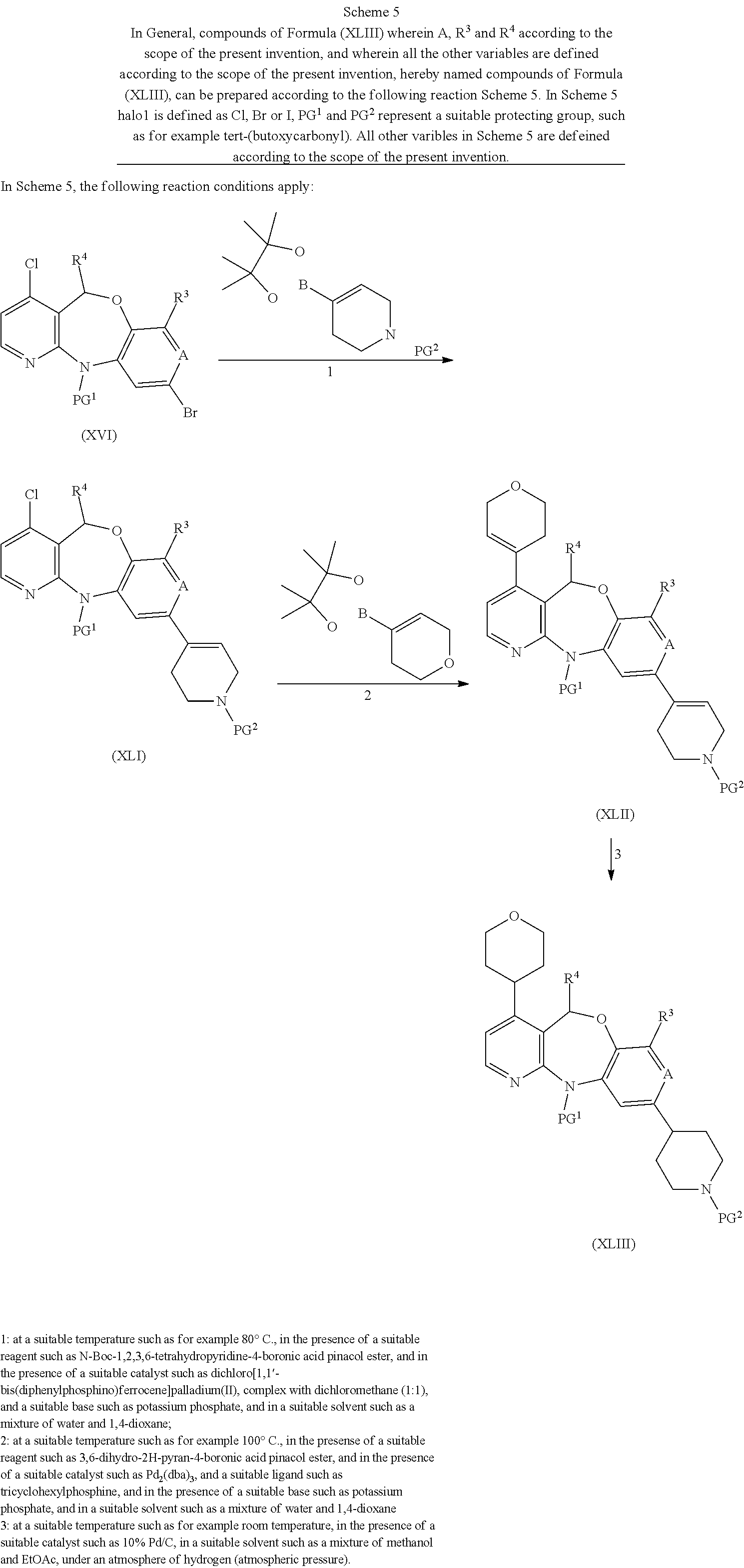

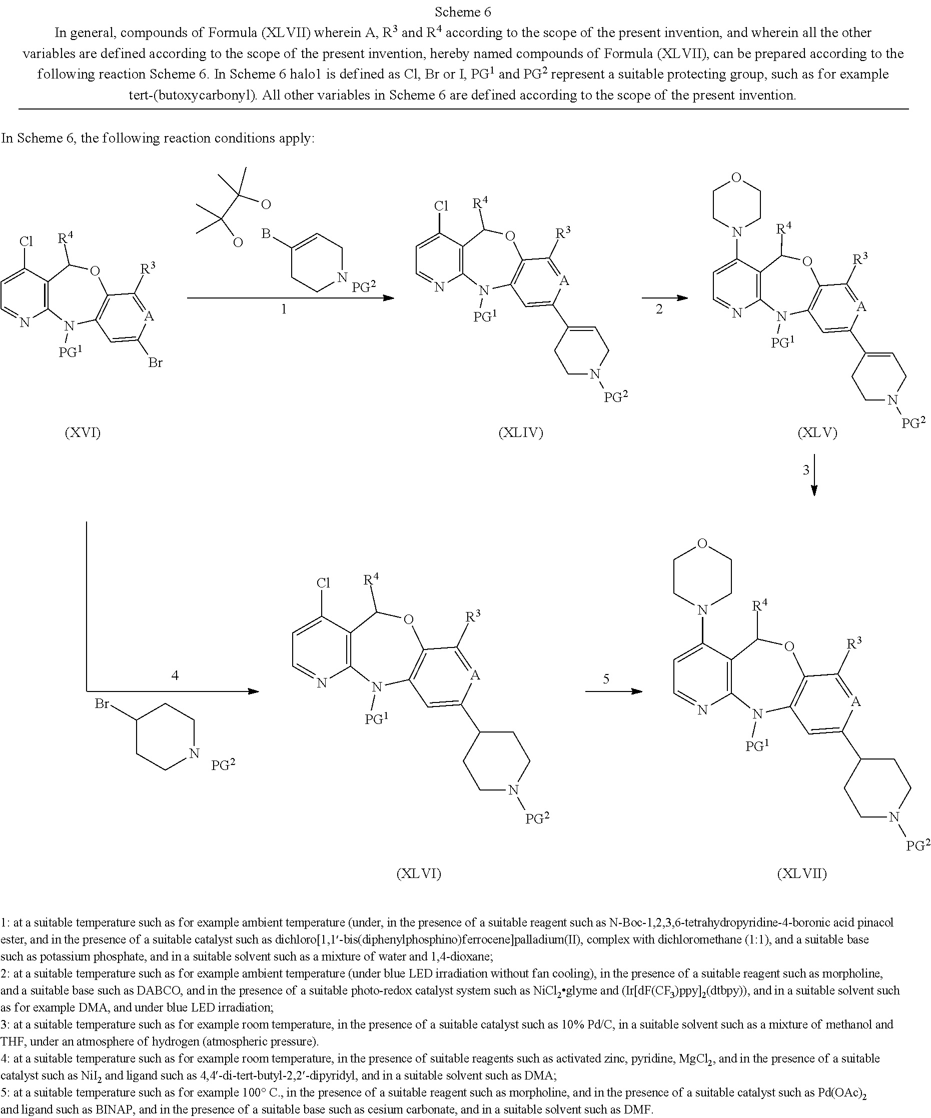

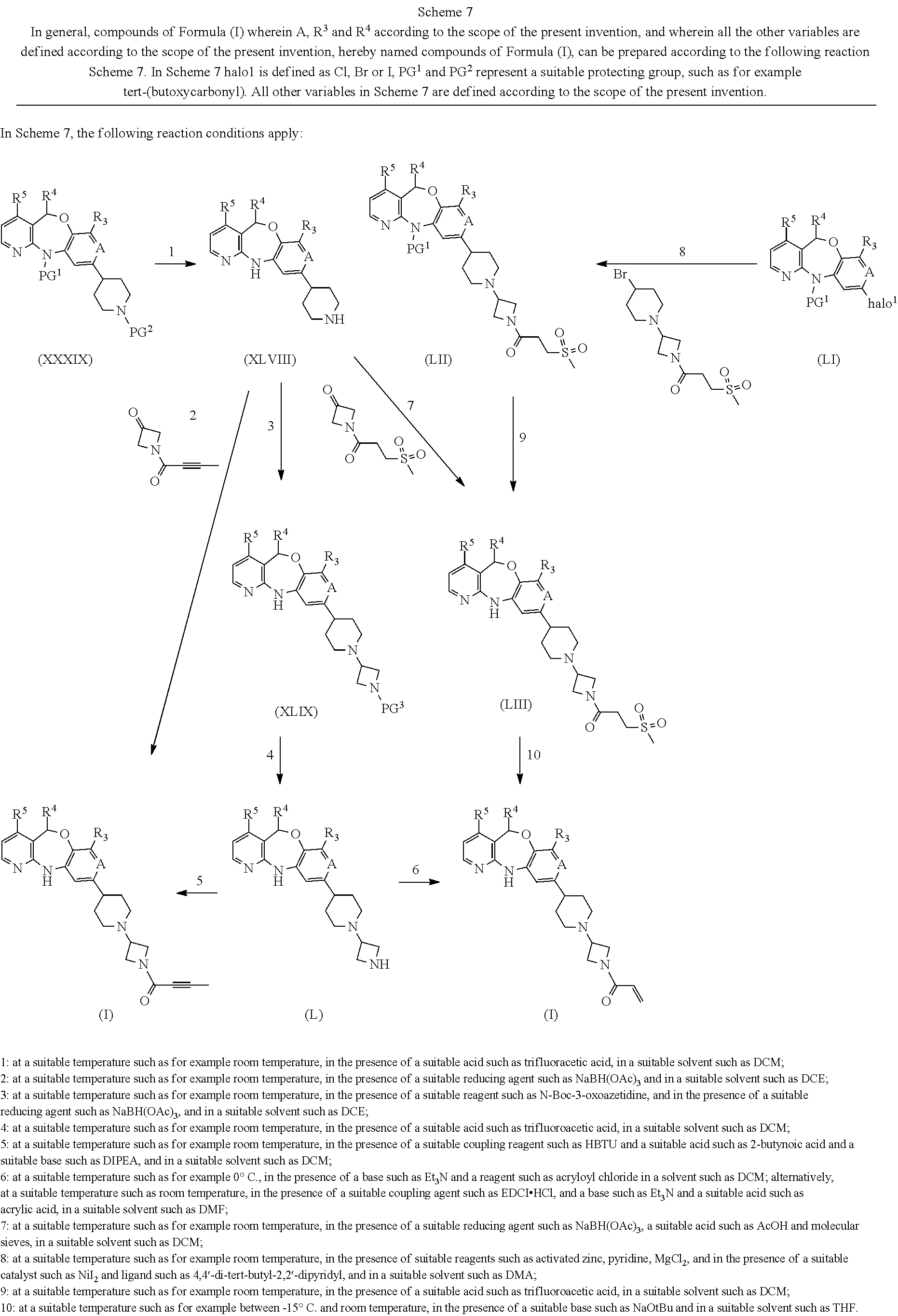

The compounds of formula (I) may also be converted into each other via art-known reactions or functional group transformations. For instance, substituents like —C(═O)—O—C₁₋₆alkyl or C₁₋₆alkyl-O—C(═O)—, can be converted into HOOC—C₁₋₆alkyl or carboxyl in the presence of lithium hydroxide, and in the presence of a suitable solvent, such as for example tetrahydrofuran or an alcohol, e.g. methanol.

The skilled person will realize that in the reactions described herein, in certain cases it may be advisable or necessary to perform the reaction under an inert atmosphere, such as for example under N₂-gas atmosphere.

It will be apparent for the skilled person that it may be necessary to cool the reaction mixture before reaction work-up, meaning those series of manipulations required to isolate and purify the product(s) of a chemical reaction such as for example quenching, column chromatography, or extraction.

The skilled person will realize that heating the reaction mixture under stirring may enhance the reaction outcome. In some reactions microwave heating may be used instead of conventional heating to shorten the overall reaction time.

The compounds of the invention as prepared in the processes described herein may be synthesized in the form of mixtures of enantiomers, in particular racemic mixtures of enantiomers, that can be separated from one another following art-known resolution procedures. Racemic compounds of formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formula (I), and the pharmaceutically acceptable addition salts and solvates thereof, involves liquid chromatography using a chiral stationary phase e.g. by supercritical fluid chromatography. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

In all these preparations, the reaction products may be isolated from the reaction medium and, if necessary, further purified according to methodologies generally known in the art such as, for example, extraction, crystallization, trituration and chromatography. The purity of the reaction products may be determined according to methodologies generally known in the art such as for example LC-MS, TLC, HPLC.

### Methods of Treatment and Medical Uses, Pharmaceutical Compositions, and Combinations

The present invention also provides methods for the treatment or prevention of a proliferative disease (e.g., cancer, benign neoplasm, angiogenesis, inflammatory disease, autoinflammatory disease, or autoimmune disease) or an infectious disease (e.g., a viral disease) in a subject. Such methods comprise the step of administering to the subject in need thereof an effective amount of a compound of the present disclosure, or a pharmaceutically acceptable salt, solvate, hydrate, tautomer, stereoisomer, or isotopically labeled derivative thereof, or a pharmaceutical composition thereof.

The subject being treated is a mammal. The subject may be a human. The subject may be a domesticated animal, such as a dog, cat, cow, pig, horse, sheep, or goat. The subject may be a companion animal such as a dog or cat. The subject may be a livestock animal such as a cow, pig, horse, sheep, or goat. The subject may be a zoo animal. The subject may be a research animal such as a rodent, dog, or non-human primate. The subject may be a non-human transgenic animal such as a transgenic mouse or transgenic pig.

The proliferative disease to be treated or prevented using the compounds of Formula (I) or Formula (II) will typically be associated with aberrant activity of CDK7. Aberrant activity of CDK7 may be an elevated and/or an inappropriate (e.g., abnormal) activity of CDK7. In certain embodiments, CDK7 is not overexpressed, and the activity of CDK7 is elevated and/or inappropriate. In certain other embodiments, CDK7 is overexpressed, and the activity of CDK7 is elevated and/or inappropriate. The compounds of the present disclosure, and pharmaceutically acceptable salts, solvates, hydrates, tautomers, stereoisomers, isotopically labeled derivatives, and compositions thereof, may inhibit the activity of CDK7 and be useful in treating and/or preventing proliferative diseases.

A proliferative disease may also be associated with inhibition of apoptosis of a cell in a biological sample or subject. All types of biological samples described herein or known in the art are contemplated as being within the scope of the invention. Inhibition of the activity of CDK7 is expected to cause cytotoxicity via induction of apoptosis. The compounds of the present disclosure, and pharmaceutically acceptable salts, solvates, hydrates, tautomers, stereoisomers, isotopically labeled derivatives, and compositions thereof, may induce apoptosis, and therefore, be useful in treating and/or preventing proliferative diseases.

Cancers that may benefit from a treatment with CDK7 inhibitors of the invention include lymphomas, leukemias, carcinomas, and sarcomas, e.g. non-Hodgkin's lymphoma, diffuse large B-cell lymphoma (DLBCL), mantle cell lymphoma (MCL), follicular lymphoma (FL), mucosa-associated lymphoid tissue (MALT) lymphoma, marginal zone lymphoma, T-cell lymphoma, Hodgkin's lymphoma, Burkitt's lymphoma, multiple myeloma, chronic lymphocytic leukemia (CLL), lymphoblastic T cell leukemia, chronic myelogenous leukemia (CML), hairy-cell leukemia, acute lymphoblastic T cell leukemia (T-ALL), Plasmacytoma, Immunoblastic large cell leukemia, megakaryoblastic leukemia, acute megakaryocytic leukemia, acute myeloid leukemia (AML) promyelocytic leukemia, erytholeukemia, brain (gliomas), glioblastomas, breast cancer, colorectal/colon cancer, prostate cancer, lung cancer including small-cell and non-small-cell, gastric cancer, endometrial cancer, melanoma, pancreatic cancer, liver cancer, kidney cancer, squamous cell carcinoma, ovarian cancer, sarcoma, osteosarcoma, thyroid cancer, bladder cancer, head&neck cancer, testicular cancer, Ewing's sarcoma, rhabdomyosarcoma, medulloblastoma, neuroblastoma, cervical cancer, renal cancer, urothelial cancer, vulval cancer, esophageal cancer, salivary gland cancer, nasopharangeal cancer, buccal cancer, cancer of the mouth, and GIST (gastrointestinal stromal tumor).

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. Generally, the amount of a compound of the present invention to be administered as a therapeutic agent for treating the disorders referred to herein will be determined on a case by case by an attending physician.

Those of skill in the treatment of such diseases could determine the effective therapeutic daily amount from the test results presented hereinafter. An effective therapeutic daily amount may be from about 0.005 mg/kg to 50 mg/kg body weight. The amount of a compound according to the present invention, also referred to here as the active ingredient, which is required to achieve a therapeutically effect may vary on case-by-case basis, for example with the particular compound, the route of administration, the age and condition of the recipient, and the particular disorder or disease being treated. A method of treatment may also include administering the active ingredient on a regimen of between one and four intakes per day. In these methods of treatment, the compounds according to the invention are preferably formulated prior to administration. As described herein below, suitable pharmaceutical formulations are prepared by known procedures using well known and readily available ingredients. While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy, for example, using methods such as those described in Gennaro et al. Remington's Pharmaceutical Sciences (18^{th} ed., Mack Publishing Company, 1990, see especially Part 8. Pharmaceutical preparations and their Manufacture). A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, or a nose spray. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions: or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The methods described herein may also comprise the additional step of administering one or more additional pharmaceutical agents in combination with the compound of the present invention, a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment inhibition of CDK7 or CDK12 and/or CDK13 induced by the inventive compounds or compositions of this invention in the biological sample or subject. Thus, the combination of the inventive compounds or compositions and the additional pharmaceutical agent(s) may be useful in treating proliferative diseases resistant to a treatment using the additional pharmaceutical agent(s) without the inventive compounds or compositions.

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation. For example, a compound according to the present invention and a therapeutic agent may be administered to the patient together in a single oral dosage composition such as a tablet or capsule, or each agent may be administered in separate oral dosage formulations.

For the treatment of the above conditions, the compounds of the invention may be advantageously employed in combination with one or more other medicinal agents, more particularly, with other anti-cancer agents or adjuvants in cancer therapy.

Examples of anti-cancer agents or adjuvants (supporting agents in the therapy) include but are not limited to:
platinum coordination compounds for example cisplatin optionally combined with amifostine, carboplatin or oxaliplatin;
taxane compounds for example paclitaxel, paclitaxel protein bound particles (Abraxane™) or docetaxel;
topoisomerase I inhibitors such as camptothecin compounds for example irinotecan, SN-38, topotecan, topotecan hcl;
topoisomerase II inhibitors such as anti-tumour epipodophyllotoxins or podophyllotoxin derivatives for example etoposide, etoposide phosphate or teniposide;
anti-tumour vinca alkaloids for example vinblastine, vincristine or vinorelbine;
anti-tumour nucleoside derivatives for example 5-fluorouracil, leucovorin, gemcitabine, gemcitabine hcl, capecitabine, cladribine, fludarabine, nelarabine;
alkylating agents such as nitrogen mustard or nitrosourea for example cyclophosphamide, chlorambucil, carmustine, thiotepa, mephalan (melphalan), lomustine, altretamine, busulfan, dacarbazine, estramustine, ifosfamide optionally in combination with mesna, pipobroman, procarbazine, streptozocin, temozolomide, uracil;
anti-tumour anthracycline derivatives for example daunorubicin, doxorubicin optionally in combination with dexrazoxane, doxil, idarubicin, mitoxantrone, epirubicin, epirubicin hcl, valrubicin;
molecules that target the IGF-1 receptor for example picropodophilin;
   tetracarcin derivatives for example tetrocarcin A;
   glucocorticoids, for example prednisone or prednisolone;
   antibodies for example trastuzumab (HER2 antibody), rituximab (CD20 antibody), gemtuzumab, gemtuzumab ozogamicin, cetuximab, pertuzumab, bevacizumab, alemtuzumab, eculizumab, ibritumomab tiuxetan, nofetumomab, panitumumab, tositumomab, CNTO 328;
estrogen receptor antagonists or selective estrogen receptor modulators or inhibitors of estrogen synthesis for example tamoxifen, fulvestrant, toremifene, droloxifene, faslodex, raloxifene or letrozole;
aromatase inhibitors such as exemestane, anastrozole, letrazole, testolactone and vorozole;
differentiating agents such as retinoids, vitamin D or retinoic acid and retinoic acid metabolism blocking agents (RAMBA) for example accutane;
DNA methyl transferase inhibitors for example azacytidine or decitabine;
antifolates for example premetrexed disodium;
antibiotics for example antinomycin D, bleomycin, mitomycin C, dactinomycin, carminomycin, daunomycin, levamisole, plicamycin, mithramycin;
antimetabolites for example clofarabine, aminopterin, cytosine arabinoside or methotrexate, azacitidine, cytarabine, floxuridine, pentostatin, thioguanine;
apoptosis inducing agents and antiangiogenic agents such as Bcl-2 inhibitors for example YC 137, BH 312, venetoclax, ABT 737, gossypol, HA 14-1, TW 37 or decanoic acid;
tubuline-binding agents for example combrestatin, colchicines or nocodazole;
kinase inhibitors (e.g. EGFR (epithelial growth factor receptor) inhibitors, MTKI (multi target kinase inhibitors), mTOR inhibitors) for example flavoperidol, imatinib mesylate, erlotinib, gefitinib, dasatinib, lapatinib, lapatinib ditosylate, sorafenib, sunitinib, sunitinib maleate, temsirolimus;
famesyltransferase inhibitors for example tipifarnib;
histone deacetylase (HDAC) inhibitors for example sodium butyrate, suberoylanilide hydroxamic acid (SAHA), depsipeptide (FR 901228), NVP-LAQ824, R306465, quisinostat, trichostatin A, vorinostat;
Inhibitors of the ubiquitin-proteasome pathway for example PS-341, Velcade (MLN-341) or bortezomib;
Yondelis;
Telomerase inhibitors for example telomestatin;
Matrix metalloproteinase inhibitors for example batimastat, marimastat, prinostat or metastat;
Recombinant interleukins for example aldesleukin, denileukin diftitox, interferon alfa 2a, interferon alfa 2b, peginterferon alfa 2b;
MAPK inhibitors;
Retinoids for example alitretinoin, bexarotene, tretinoin;
Arsenic trioxide;
Asparaginase;
Steroids for example dromostanolone propionate, megestrol acetate, nandrolone (decanoate, phenpropionate), dexamethasone;
Gonadotropin releasing hormone agonists or antagonists for example abarelix, goserelin acetate, histrelin acetate, leuprolide acetate;
Thalidomide, lenalidomide;
   Mercaptopurine, mitotane, pamidronate, pegademase, pegaspargase, rasburicase;
   BH3 mimetics for example ABT-199;
MEK inhibitors for example PD98059, AZD6244, CI-1040;
colony-stimulating factor analogs for example filgrastim, pegfilgrastim, sargramostim; erythropoietin or analogues thereof (e.g. darbepoetin alfa); interleukin 11; oprelvekin; zoledronate, zoledronic acid; fentanyl; bisphosphonate; palifermin;
a steroidal cytochrome P450 17alpha-hydroxylase-17,20-lyase inhibitor (CYP17), e.g. abiraterone, abiraterone acetate;
mTOR inhibitors such as rapamycins and rapalogs, and mTOR kinase inhibitors;
PI3K inhibitors and dual mTOR/PI3K inhibitors; PI3K delta inhibitors for example idelalisib and duvelisib;
BTK inhibitors for example Ibrutinib, ONO-4059, ACP-196;
R-CHOP (Rituxan added to CHOP—Cyclophosphamide, Doxorubicin, Vincristine and Prednisolone);
daratumumab;
BRD4 inhibitors;
CDK9 inhibitors;
SYK inhibitors;
PKC inhibitors;
JAK inhibitors;
PIM kinase inhibitors;
immune cell redirection agents (e.g. Blinatumomab or CAR T cells); and
immunomodulatory agents (e.g. anti-PD1 antibodies).

Therefore, an embodiment of the present invention relates to a product containing as first active ingredient a compound according to the invention and as further active ingredient one or more anticancer agent, as a combined preparation for simultaneous, separate or sequential use in the treatment of patients suffering from cancer.

The one or more other medicinal agents and the compound according to the present invention may be administered simultaneously (e.g. in separate or unitary compositions) or sequentially in either order. In the latter case, the two or more compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular other medicinal agent and compound of the present invention being administered, their route of administration, the particular tumour being treated and the particular host being treated. The optimum method and order of administration and the dosage amounts and regime can be readily determined by those skilled in the art using conventional methods and in view of the information set out herein.

The weight ratio of the compound according to the present invention and the one or more other anticancer agent(s) when given as a combination may be determined by the person skilled in the art. Said ratio and the exact dosage and frequency of administration depends on the particular compound according to the invention and the other anticancer agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of Formula (I) and another anticancer agent may range from 1/10 to 10/1, more in particular from 1/5 to 5/1, even more in particular from 1/3 to 3/1.

### EXAMPLES

The following examples are offered for purposes of illustration and are not intended to limit the scope of the claims provided herein. All literature citations in these examples and throughout this specification are incorporated herein by references for all legal purposes to be served thereby. The starting materials and reagents used for the synthesis of the compounds described herein may be synthesized or can be obtained from commercial sources, such as, but not limited to, Sigma-Aldrich, Acros Organics, Fluka, and Fischer Scientific.

When a stereocenter is indicated with ‘RS’ this means that a racemic mixture was obtained.

For intermediates that may be used in a next reaction step as a crude or as a partially purified intermediate, theoretical mol amounts may be indicated in the reaction protocols described below.

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

Hereinafter, the terms: ‘ACN’ means acetonitrile, ‘AcOH’ means acetic acid, ‘Ar’ means argon, ‘BINAP’ means 2,2′-bis(diphenylphosphino)-1,1′-binaphthyl, ‘BOC’ means tert-butyloxycarbonyl, ‘Boc₂O’ means di-tert-butyl dicarbonate, ‘Celite®’ means diatomaceous earth, ‘DCM’ means dichloromethane, ‘DIPEA’ means diisopropylethylamine, ‘h’ means hours(s), ‘min’ means minute(s), ‘Int.’ means intermediate; ‘aq.’ Means aqueous; ‘DMAP’ means dimethylaminopyridine, ‘DMF’ means dimethylformanide, ‘Et₂O’ means diethylether, ‘EtOAc’ means ethyl acetate, ‘HPLC’ means High-performance Liquid Chromatography, ‘iPrOH’ means isopropyl alcohol, ‘HATU’ means 1-[bis(dimethylamino)methylene]-1H-[1,2,3]triazolo[4,5-b]pyridin-1-ium 3-oxide hexafluorophosphate, ‘LC/MS’ means Liquid Chromatography/Mass Spectrometry, ‘Me-THF’ means methyl-tetrahydrofuran, ‘MeOH’ means methanol, ‘EtOH’ means ethanol, ‘NBS’ means N-bromosuccinimide, ‘NCS’ means N-chlorosuccinimide, ‘NMR’ means Nuclear Magnetic Resonance, ‘Pd/C 10%’ means palladium on carbon loading 10%, ‘Pd(OAc)₂’ means palladium (II) acetate, ‘Pd(PPh₃)₄’ means tetrakis(triphenylphosphine)palladium (0), ‘rt’ means room temperature, ‘SFC’ means supercritical fluid chromatography, ‘ee’ means enantiomeric excess, ‘TBAF’ means tetrabutylanmonium fluoride, ‘TBDMS’ or ‘SMDBT’ means tert-butyldimethylsilyl, ‘TEA’ means triethylamine, ‘TFA’ means trifluoroacetic acid, ‘TiF’ means tetrahydrofuran, ‘CV’ means column volumes, ‘Quant.’ means quantitative, ‘equiv.’ means equivalent(s), ‘M.P.’ or ‘m.p.’ means melting point, ‘OR’ means optical rotation, ‘DIPE’ means diisopropyl ethylether, ‘RaNVi’ means Raney Nickel, ‘NaHCO₃’ means sodium hydrogenocarbonate, ‘BRETTPHOS’ means 2-(dicyclohexylphosphino)-3,6-dimethoxy-2′,4′,6′-triisopropyl-1,1′-biphenyl, ‘DMSO’ means dimethylsulfoxide, ‘NaBH₃(OAc)₃’ means sodium triacetoxyborohydride, ‘DMA-DMF’ means N,N-dimethylformaniidedimethylacetal, ‘v/v’ means volume/volume percent, ‘T’ means temperature, ‘iPrNH₂’ means isopropylamine.

### Example A: Preparation of Intermediates and Final Compounds

### Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

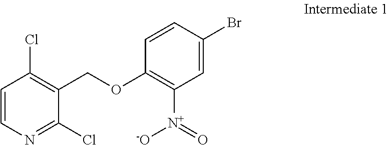

To a solution of (2,4-dichloropyridin-3-yl)methanol (CAS [945543-24-8], 8.0 g, 44.940 mmol, 1 eq.) in THF (200 mL) was added 4-bromo-2-nitrophenol (9.797 g, 44.940 mmol, 1 eq.), PPh₃ (35.362 g, 134.819 mmol, 3 eq.) and this was followed by the addition of DIAD (27.262 g, 134.819 mmol, 3 eq.). The reaction mixture was stirred for 4 h at room temperature under a nitrogen atmosphere. The reaction was quenched with water (200 mL). The resulting mixture was extracted with EtOAc (3×300 mL). The organic layers were combined, dried over anhydrous sodium sulfate, filtered, and concentrated. The residue was purified by silica gel chromatography (0-10% EtOAc/DCM) to afford Intermediate 1 as a yellow solid (13.5 g, yield: 79%).

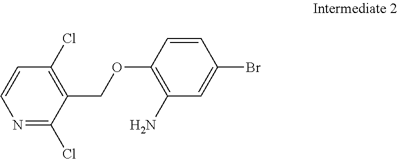 [SMILES: Nc1cc(Br)ccc1OCc1c(Cl)ccnc1Cl]

Intermediate 1 (12 g, 31.746 mmol), AcOH (50 mL) and iron powder (17.730 g, 317.455 mmol, 10.0 eq.) were stirred in MeOH (300 mL) at room temperature for 3 h. The crude mixture was diluted with EtOAc and ice was added. A saturated aqueous NaHCO₃ solution was added slowly until basic pH. The combined organic layers were dried (MgSO₄), filtered, and concentrated to give Intermediate 2 (8.15 g, yield: 74%), used without further purification.

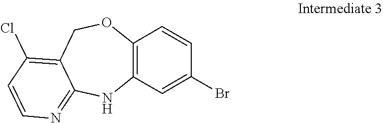 [SMILES: Clc1ccnc2c1COc1ccc(Br)cc1N2]

To a solution of Intermediate 2 (8.0 g, 22.987 mmol) in 1,4-dioxane (100 mL) was added TFA (7.863 mg, 68.961 mmol, 3 eq.). The reaction mixture was stirred at 120° C. for 3 h. The reaction mixture was diluted with EtOAc and washed with aqueous NaHCO₃. The organic layer was dried with MgSO₄ and concentrated. The residue was purified by silica gel chromatography (0-50% EtOAc/petroleum ether) to afford Intermediate 3 as a white solid (4.5 g, yield: 63%).

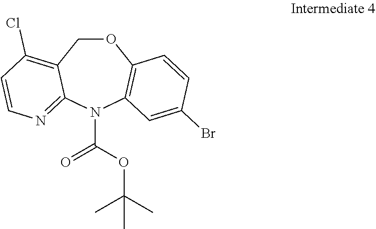 [SMILES: CC(C)(C)OC(=O)N1c2cc(Br)ccc2OCc2c(Cl)ccnc21]

NaH (60% dispersion in mineral oil, 1.354 g, 33.86 mmol, 2.11 eq.) was added portionwise at 5° C. to a solution of Intermediate 3 (5 g, 16.048 mmol) in THF (105 mL). The reaction mixture was stirred for 45 min at 0-5° C. Di-tert-butyl decarbonate (7.398 g, 33.899 mmol, 2.11 eq.) was added. The reaction mixture was stirred for 3 h at room temperature. The mixture was then poured onto ice. Water was added and the mixture was extracted twice with DCM, dried over MgSO₄, filtered, and evaporated. The residue was purified by column chromatography (Stationary phase irregular SiOH 15-40 μm 80 g GraceResolv®, Mobile phase: heptane/EtOAc 90/10 to 60/40). The collected fractions were combined and evaporated. The residue was taken up in DIPE, filtered, and dried to afford Intermediate 4 (3.8 g, yield: 57%).

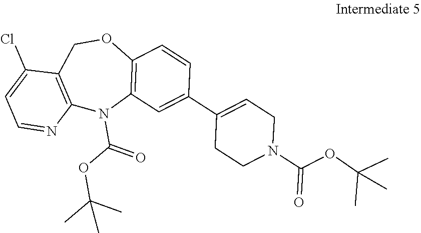 [SMILES: CC(C)(C)OC(=O)N1CC=C(c2ccc3c(c2)N(C(=O)OC(C)(C)C)c2nccc(Cl)c2CO3)CC1]

A mixture of Intermediate 4 (26 g, 63.156 mmol), N-Boc-1,2,3,6-tetrahydropyridine-4-boronic acid pinacol ester (CAS [286961-14-6], 19.6 g, 63.387 mmol, 1 eq.), Pd(dppf)Cl₂·DCM (CAS [95464-05-4], 5.6 g, 6.211 mmol, 0.1 eq.) in aqueous Na₂CO₃ (126 mL, 1 M, 126 mmol, 2 eq.) and 1,4-dioxane (400 mL) was stirred for 3.5 h at 80° C. under nitrogen flux. After cooling, the mixture was poured into water and EtOAc. This mixture was filtered over a pad of Celite® and the Celite® was washed three times with EtOAc. The organic layer was separated and evaporated. The residue was purified by column chromatography (Irregular SiOH 15-40 μm 330 g GraceResolv®, mobile phase heptane/EtOAc 90/10 to 60/40) to afford Intermediate 5 (23.4 g, yield: 73%).

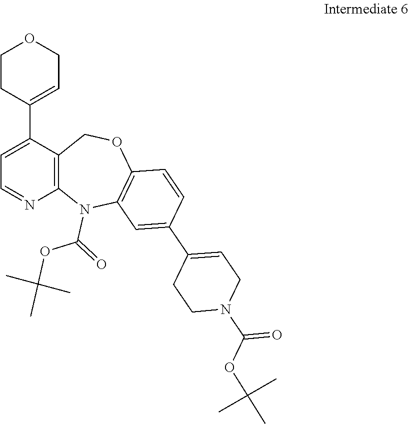 [SMILES: CC(C)(C)OC(=O)N1CC=C(c2ccc3c(c2)N(C(=O)OC(C)(C)C)c2nccc(C4=CCOCC4)c2CO3)CC1]

A mixture of Intermediate 5 (14.4 g, 28.014 mmol), 3,6-dihydro-2H-pyran-4-boronic acid pinacol ester (CAS [287944-16-5], 6.2 g, 29.512 mmol, 1.05 eq.), potassium phosphate (12 g, 56.533 mmol, 2 eq.), tricyclohexylphosphine (1.9 g, 6.775 mmol, 0.24 eq.) and Pd₂(dba)₃ (CAS [52409-22-0], 2.5 g, 2.73 mmol, 0.1 eq.) in 1,4-dioxane (210 mL) and water (30 mL) was stirred at 100° C. for 3 h. After cooling, the reaction mixture was poured into water and was extracted twice with EtOAc. The combined organic layer was evaporated and the residue was purified by column chromatography (Irregular SiOH 15-40 μm 330 g GraceResolv®, mobile phase gradient heptane/EtOAc 70/30 to 40/60) to afford pure Intermediate 6 (5.84 g, yield: 37%) and an impure fraction. This impure fraction was purified again by column chromatography (Irregular SiOH 15-40 μm 120 g GraceResolv®, mobile phase gradient heptane/EtOAc 70/30 to 40/60) to afford another batch of Intermediate 6 (4.30 g, yield: 27%).

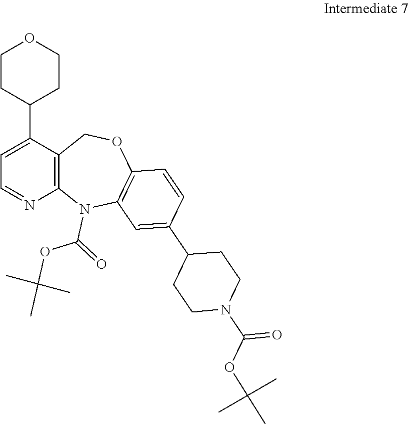 [SMILES: CC(C)(C)OC(=O)N1CCC(c2ccc3c(c2)N(C(=O)OC(C)(C)C)c2nccc(C4CCOCC4)c2CO3)CC1]

Intermediate 6 (5.84 g, 10.397 mmol) was hydrogenated in the presence of Pd/C (10%, 5.4 g, 5.074 mmol, 0.49 eq.) as a catalyst in MeOH (50 mL) and EtOAc (140 mL) for 5 h at atmospheric pressure and at room temperature. The catalyst was filtered off over Celite® and the Celite® was washed three times with a mixture of MeOH/EtOAc (50/50). The solvent was evaporated to afford Intermediate 7 (5.88 g, quantitative), used without further purification.

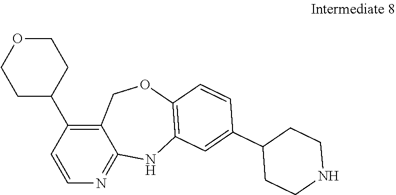 [SMILES: c1cc(C2CCOCC2)c2c(n1)Nc1cc(C3CCNCC3)ccc1OC2]

At 0° C. TFA (12 mL, 156.809 mmol, 15 eq.) was added slowly to a solution of Intermediate 7 (5.88 g, 10.394 mmol) in DCM (80 mL). The reaction mixture was stirred for 6 h. The volatiles were evaporated and the residue was taken up in DCM. A mixture of MeOH/NH₄OH (30% in water) was added at 0° C. More water was added and the organic layer was separated, dried over MgSO₄, filtered, and evaporated to give Intermediate 8 (3.40 g, yield: 90%), used without further purification.

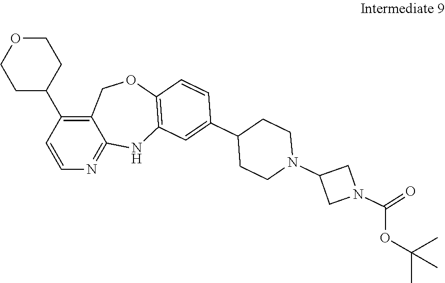 [SMILES: CC(C)(C)OC(=O)N1CC(N2CCC(c3ccc4c(c3)Nc3nccc(C5CCOCC5)c3CO4)CC2)C1]

A solution of Intermediate 8 (3.4 g, 9.303 mmol), 1-Boc-3-azetidinone (2.4 g, 14.019 mmol, 1.5 eq.), NaBH(OAc)₂ (3.9 g, 18.401 mmol, 2 eq.), AcOH (0.94 mL, 16.42 mmol, 1.76 eq.) in dry DCM (50 mL) was stirred at room temperature overnight. The reaction was quenched with an aqueous solution of K₂CO₃ (10%) and the mixture was extracted twice with EtOAc. The organic layer was separated and evaporated. The residue was taken up in EtOH, triturated, and filtered. The precipitate was washed once with EtOH and dried to give Intermediate 9 (3.50 g, yield: 72%), used without further purification.

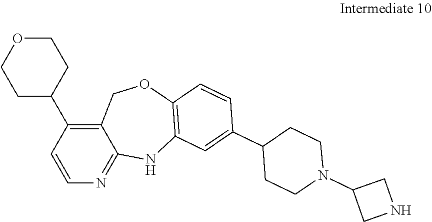 [SMILES: c1cc(C2CCOCC2)c2c(n1)Nc1cc(C3CCN(C4CNC4)CC3)ccc1OC2]

At 0° C., HCl (17 mL, 4 M in dioxane, 68 mmol, 10.4 eq.) was added slowly to a solution of Intermediate 9 (3.4 g, 6.53 mmol) in 1,4-dioxane (90 mL) and MeOH (10 mL). The reaction mixture was stirred for 24 h at room temperature. The solvent was evaporated and the residue was taken up in DCM (800 mL) and basified with an aqueous solution of Na₂CO₃ (400 mL, 1 M). This heterogenous mixture was stirred for 15 min at room temperature. The layers were separated and the organic laye was evaporated. The residue was taken up in ACN, triturated, and filtered. The precipitate was dried to give Intermediate 10 (1.75 g, yield: 64%), used without further purification.

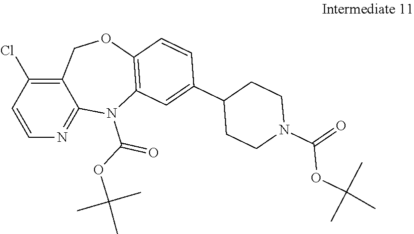 [SMILES: CC(C)(C)OC(=O)N1CCC(c2ccc3c(c2)N(C(=O)OC(C)(C)C)c2nccc(Cl)c2CO3)CC1]

[1-(tert-Butoxycarbonyl)piperidin-4-yl]zinc iodide (CAS [807618-13-9], 76 mL, 0.45 M in THF, 34.2 mmol, 2 eq.) was added to a stirred solution of Intermediate 4 (7 g, 17 mmol), 1,1′-bis(diphenylphosphino)ferrocene-palladium(II)dichloride DCM adduct (CAS [1003846-21-6], 1.4 g, 1.695 mmol, 0.1 eq.), and copper(I) iodide (420 mg, 2.205 mmol, 0.13 eq.) in DMA (80 mL) in a sealed tube under nitrogen atmosphere. The mixture was stirred at 80° C. for 1 h. After cooling the reaction was quenched with aqueous saturated NH₄Cl and the mixture was extracted with EtOAc. The organic layer was separated, dried (Na₂SO₄), filtered, and evaporated. The residue was purified by flash column chromatography (silica (dry load), heptane/EtOAc 90/10 to 50/50) to afford Intermediate 11 (5.87 g, yield: 67%) as a white foam.

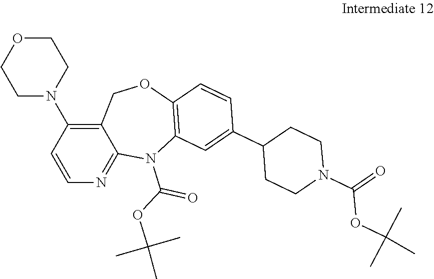 [SMILES: CC(C)(C)OC(=O)N1CCC(c2ccc3c(c2)N(C(=O)OC(C)(C)C)c2nccc(N4CCOCC4)c2CO3)CC1]

Intermediate 11 (4 g, 7.752 mmol), morpholine (1.34 mL, 15.502 mmol, 2 eq.), palladium(II) acetate (174 mg, 0.776 mmol, 0.1 eq.), 2,2′-bis(diphenylphosphino)-1,1′-binaphthyl (CAS [98327-87-8], 966 mg, 1.550 mmol, 0.2 eq.) and cesium carbonate (6.314 g, 19.378 mmol, 2.5 eq.) in 1,4-dioxane (60) in a sealed tube were stirred at 105° C. for 12 h. The reaction mixture was poured into 10% NH₄Cl aqueous solution, extracted twice with DCM, dried over MgSO₄, filtered, and evaporated. The residue was purified by chromatography over silica gel (SiO₂ 15-40 μm, GraceResolv®, 80 g; eluent: heptane/EtOAc/2% NH₄OH in MeOH 80/20/0 to 45/50/5) to give Intermediate 12 (4.2 g, yield: 96%).

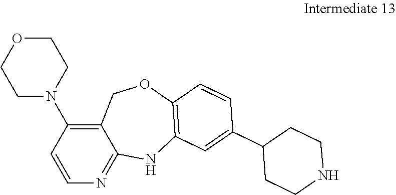 [SMILES: c1cc(N2CCOCC2)c2c(n1)Nc1cc(C3CCNCC3)ccc1OC2]

At 0° C., TFA (73.5 mL, 960.456 mmol, 130 eq.) was added to a stirred solution of Intermediate 12 (4.2 g, 7.411 mmol) in DCM (147 mL). The reaction mixture was stirred at room temperature for 5 h. The solvent was evaporated and the residue was poured onto ice. Water and NH₄OH were added until basic pH. The mixture was extracted twice with DCM, The organic layer was filtered on Chromabond® and the solvent was evaporated to give Intermediate 13 (2.71 g, quantitative), used without further purification.

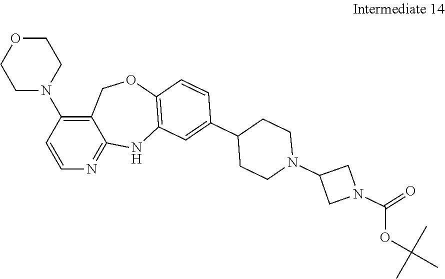 [SMILES: CC(C)(C)OC(=O)N1CC(N2CCC(c3ccc4c(c3)Nc3nccc(N5CCOCC5)c3CO4)CC2)C1]

A solution of Intermediate 13 (2.71 g, 7.395 mmol), 1-Boc-3-azetidinone (CAS [398489-26-4], 1899 mg, 11.092 mmol, 1.5 eq.), AcOH (757 μL 13.214 mmol, 1.79 eq.) and NaBH(OAc)₃ (3.188 g, 15.043 mmol, 2 eq.) in dry DCM (25 mL) was stirred at room temperature overnight. Water was added and the mixture was basified with aqueous NH₄OH. The mixture was extracted twice with DCM. The organic layer was dried over MgSO₄, filtered, and evaporated. The residue was purified by column chromatography (80 g of SiOH 35-40 μm GraceResolv, gradient DCM/0.1% NH₄OH I MeOH 100/0 to 93/7) to give Intermediate 14 (3300 mg, yield: 85%).

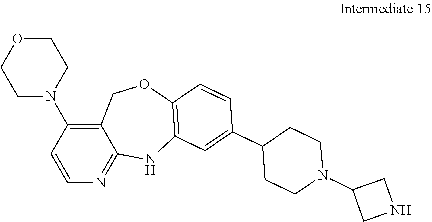 [SMILES: c1cc(N2CCOCC2)c2c(n1)Nc1cc(C3CCN(C4CNC4)CC3)ccc1OC2]

A mixture of Intermediate 14 (3.3 g, 6.326 mmol) and HCl (15.897 mL, 4 M in dioxane, 63.588 mmol, 10 eq.) in 1,4-dioxane (87 mL) and EtOH (II mL) was stirred at room temperature for 12 h. The volatiles were evaporated to afford Intermediate 15 (3.2 g, quantitative), used without further purification.

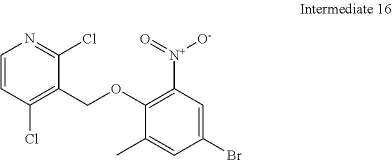 [SMILES: Cc1cc(Br)cc([N+](=O)[O-])c1OCc1c(Cl)ccnc1Cl]

2,4-Dichloro-3-pyridinemethanol (CAS [945543-24-8], 1 g, 5.505 mmol), 4-bromo-2-methyl-6-nitrophenol (CAS [20294-50-2], 1.277 g, 5.505 mmol, 1 eq.), and triphenylphosphine (4.332 g, 16.515 mmol, 3 eq.) were mixed in dry THF (37 mL) under nitrogen atmosphere. Then, DIAD (CAS [2446-83-5], 3.25 mL, 16.515 mmol, 3 eq.) was added dropwise and the reaction mixture was stirred at room temperature for 30 min. The reaction mixture was partitioned between EtOAc and brine. The combined organic layers were dried (MgSO₄), filtered, and concentrated. The residue was purified by column flash chromatography (silica; heptane:DCM 9:1/EtOAc gradient) to afford Intermediate 16 (2.125 g, yield: 98%).

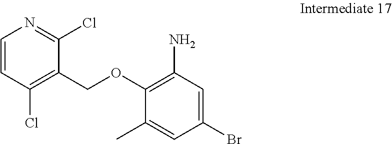 [SMILES: Cc1cc(Br)cc(N)c1OCc1c(Cl)ccnc1Cl]

A mixture of Intermediate 16 (2.125 g, 5.312 mmol), iron powder (2.996 g, 53.12 mmol, 10 eq.), and AcOH (6.08 mL, 106.241 mmol, 20 eq.) in MeOH (42 mL) was stirred at room temperature for 2 h. The crude mixture was dissolved in EtOAc, ice was added, followed by saturated aqueous NaHCO₃ until basic pH. The layers were separated and the combined organic layers were dried (MgSO₄), filtered, and concentrated to give Intermediate 17 (1.915 g, quantitative), used without further purification.

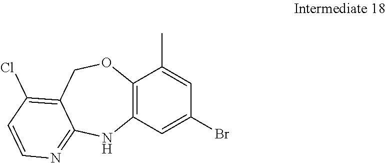 [SMILES: Cc1cc(Br)cc2c1OCc1c(Cl)ccnc1N2]

Intermediate 17 (1.915 g, 5.289 mmol) and TFA (1.21 mL, 15.868 mmol, 3 eq.) were dissolved in 1,4-dioxane (26 mL) and the reaction mixture was stirred at 120° C. for 2 h. The mixture was cooled to room temperature and diluted with EtOAc, then washed with aqueous NaHCO₃ and brine. The organic layer was dried (MgSO₄), filtered, and concentrated. The residue was purified by column flash chromatography (SiO₂, EtOAc/heptane gradient) to afford Intermediate 18 (1325 g, yield: 77%).

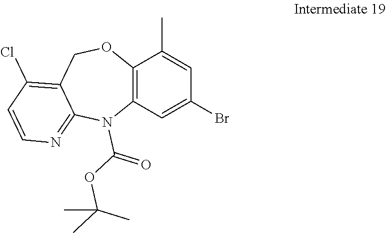 [SMILES: Cc1cc(Br)cc2c1OCc1c(Cl)ccnc1N2C(=O)OC(C)(C)C]

To a solution of Intermediate 18 (1.325 g, 3.855 mmol), DMAP (239 mg, 1.933 mmol, 0.5 eq.), and Et₃N (1.62 mL, 11.598 mmol, 3 eq.) in DCM (20 mL), di-tert-butyl decarbonate (2.53 g, 11.598 mmol, 3 eq.) was added and the mixture was stirred at room temperature for 20 h. The mixture was directly purified by column flash chromatography (SiO₂, heptane/EtOAc gradient) to afford Intermediate 19 (1.4 g, yield: 85%).

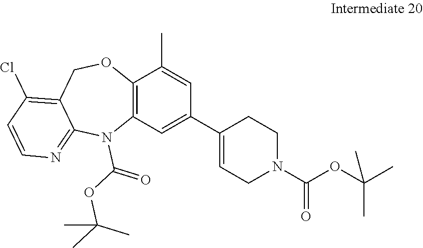 [SMILES: Cc1cc(C2=CCN(C(=O)OC(C)(C)C)CC2)cc2c1OCc1c(Cl)ccnc1N2C(=O)OC(C)(C)C]

In a sealed tube, a solution of Intermediate 19 (920 mg, 2.14 mmol), N-Boc-1,2,3,6-tetrahydropyridine-4-boronic acid pinacol ester (CAS [286961-14-6], 662 mg, 2.14 mmol, 1 eq.), and potassium phosphate (908 mg, 4.279 mmol, 2 eq.) in 1,4-dioxane (15 mL) and water (2 mL) was degassed under nitrogen atmosphere. Then dichloro[1,1-bis(diphenylphosphino)ferrocene]palladium(II), complex with DCM (1:1) (CAS [95464-05-4], 175 mg, 0.214 mmol, 0.1 eq.) was added. The reaction mixture was degassed again with nitrogen and was then stirred at 80° C. for 3 h. The reaction mixture was partitioned between EtOAc and brine. The combined organic layers were dried (MgSO₄), filtered, and concentrated. The residue was purified by column flash chromatography (silica; heptane/EtOAc gradient) to give Intermediate 20 (565 mg, yield: 50%).

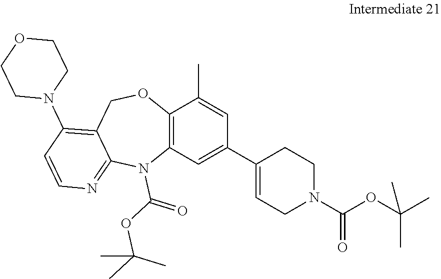 [SMILES: Cc1cc(C2=CCN(C(=O)OC(C)(C)C)CC2)cc2c1OCc1c(N3CCOCC3)ccnc1N2C(=O)OC(C)(C)C]

A solution of Intermediate 20 (846 mg, 1.442 mmol), morpholine (189 μL, 2.163 mmol, 1.5 eq.), and DABCO (333 mg, 2.884 mmol, 2 eq.) in dry DMA (25 mL) was degassed with nitrogen. Then, NiCl₂.glyme (CAS [29046-78-4], 32 mg, 0.144 mmol, 0.1 eq.) and (Ir[dF(CF₃)ppy]₂(dtbpy)) (CAS [870987-63-61, 3 mg, 0.003 mmol, 0.002 eq.) were added and the mixture was degassed for 1 min. The reaction mixture was stirred under blue LED irradiation without fan cooling for 16 h. A new recharge of NiCl₂.glyme (16 mg, 0.77 mmol, 0.5 eq.) and (Ir[dF(CF₃)ppy]₂(dtbpy)) (1.5 mg, 0.0015 mmol, 0.001 eq.) was added and mixture was stirred under blue LED irradiation without fan cooling for 3 days. The reaction mixture was partitioned between EtOAc and a saturated aqueous NaHCO₃ solution. The layers were separated and the combined organic layers were dried (MgSO₄), filtered, and concentrated. The residue was purified by column flash chromatography on silica gel to give Intermediate 21 (498 mg, yield: 60%).

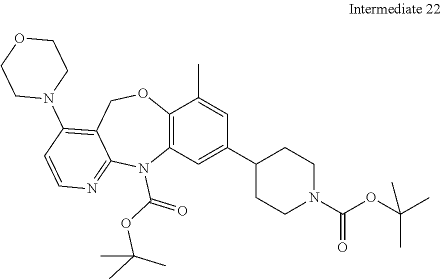 [SMILES: Cc1cc(C2CCN(C(=O)OC(C)(C)C)CC2)cc2c1OCc1c(N3CCOCC3)ccnc1N2C(=O)OC(C)(C)C]

Pd/C 10% (38 mg) was added to a solution of Intermediate 21 (498 mg, 0.764 mmol) in a mixture of THF (15 mL) and MeOH (15 mL) under nitrogen atmosphere and the mixture was purged with nitrogen, then with hydrogen. The reaction mixture was stirred under hydrogen atmosphere for 15 h. The reaction mixture was filtered through a short pad of Celite and the cake washed with MeOH and DCM. The combined filtrate was concentrated to afford Intermediate 22 (434 mg, yield: 98%), used without further purification.

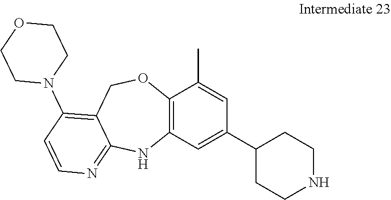 [SMILES: Cc1cc(C2CCNCC2)cc2c1OCc1c(N3CCOCC3)ccnc1N2]

TFA (3 mL, 38.6 mmol, 40 eq.) was added to a solution of Intermediate 22 (561 mg, 0.966 mmol) in DCM (15 mL) stirred at 0° C. and the reaction mixture was then stirred at room temperature for 3 h. The reaction mixture was concentrated and the residue was poured in water and basified with saturated aqueous Na₂CO₃ and extracted with DCM. The organic layer was dried over MgSO₄, filtered, and evaporated to afford Intermediate 23 (368 mg, quantitative), used without further purification.

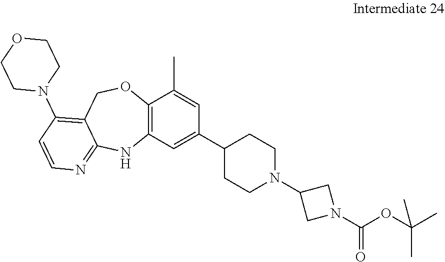 [SMILES: Cc1cc(C2CCN(C3CN(C(=O)OC(C)(C)C)C3)CC2)cc2c1OCc1c(N3CCOCC3)ccnc1N2]

To a solution of Intermediate 23 (368 mg, 0.966 mmol) in MeOH (20 mL) were added N-Boc-3-oxoazetidine (CAS [398489-26-4], 248 mg, 1.449 mmol, 1.5 eq.), and AcOH (66 μL, 1.159 mmol, 1.2 eq.). The reaction mixture was stirred at room temperature for 30 min; then NaBH₃CN (61 mg, 0.966 mmol, 1 eq.) was added. The reaction mixture was stirred at room temperature for 16 h. An additional 0.5 eq. of each N-Boc-3-oxoazetidine, AcOH, and NaBH₃CN was added and the reaction mixture was stirred at room temperature overnight. To push the reaction to completion, a new recharge of 0.5 eq. of each N-Boc-3-oxoazetidine, AcOH, and NaBH₃CN was added again. The reaction mixture was washed with aqueous NaHCO₃ and extracted with EtOAc. The organic layer was washed with aqueous NaHCO₃, dried over MgSO₄, filtered, and evaporated. The residue was purified by column flash chromatography on silica gel (hexane/EtOAc gradient) to give Intermediate 24 (381 mg, yield: 74%).

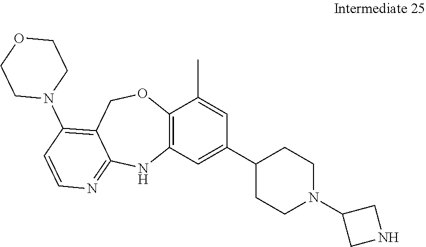 [SMILES: Cc1cc(C2CCN(C3CNC3)CC2)cc2c1OCc1c(N3CCOCC3)ccnc1N2]

TFA (2.18 mL, 28.45 mmol, 40 eq.) was added to a solution of Intermediate 24 (381 mg, 0.711 mmol) in DCM (11 mL) at 0° C. and the reaction mixture was stirred at room temperature for 3 h. The volatiles were evaporated to give Intermediate 25 (310 mg, quantitative), used without further purification.

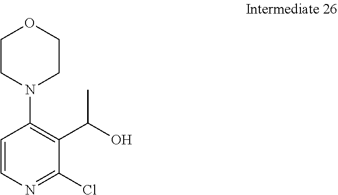 [SMILES: CC(O)c1c(N2CCOCC2)ccnc1Cl]

2-Chloro-4-(4-morpholinyl)-3-pyridinecarboxaldehyde (CAS [877054-85-8], 10 g, 42.795 mmol) was dissolved in dry TIF (175 mL) and the reaction mixture was cooled to −78° C. Methyl magnesium bromide (45.9 mL, 64.19 mmol, 1.5 eq.) was added dropwise at −78° C. and the reaction mixture was stirred at −78° C. for 1 h. More methyl magnesium bromide (6.1 mL, 8.56 mmol, 0.2 eq.) was added dropwise at −78° C. and the reaction mixture was stirred at −78° C. for 1 h. The reaction was quenched with saturated aqueous NH₄Cl and EtOAc was added. The layers were separated and the combined organic layers were dried (MgSO₄), filtered, and concentrated. The residue was purified by column flash chromatography (SiO₂, heptane/EtOAc) to give Intermediate 26 (6.916 g, yield: 67%).

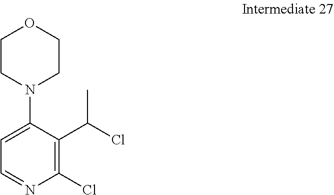 [SMILES: CC(Cl)c1c(N2CCOCC2)ccnc1Cl]

Intermediate 26 (1.2 g, 4.944 mmol) was dissolved in DCM (36 mL) at 0° C. and SOCl₂ (538 μL, 7.417 mmol, 1.5 eq.) was slowly added. The reaction mixture was stirred at room temperature for 3 h. The reaction was quenched by pouring into a stirred mixture of water/ice/DCM. The mixture was then neutralized with NaHCO₃. The layers were separated and the organic layer was dried with MgSO₄, filtered, and concentrated to afford Intermediate 27 (1.17 g, yield: 91%).

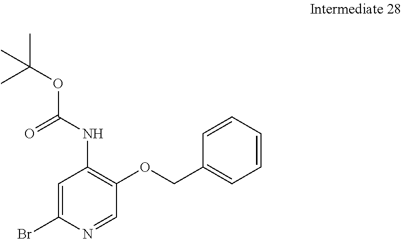 [SMILES: CC(C)(C)OC(=O)Nc1cc(Br)ncc1OCc1ccccc1]

A mixture of 2-bromo-5-(phenylmethoxy)-4-pyridinecarboxylic acid (CAS [256823-39-8], 14 g, 45.436 mmol) and DIPEA (23.74 mL, 136.307 mmol, 3 eq.) was dissolved in tBuOH (91 mL) and 1,4-dioxane (183 mL) under nitrogen atmosphere. DPPA (CAS[26386-88-9], 19.58 mL, 90.872 mmol, 2 eq.) was added and the mixture was stirred at 110° C. for 4 h. The mixture was diluted with EtOAc and washed with aqueous NaHCO₃ and brine. The organic layer was concentrated and the residue was purified by flash column chromatography (SiO₂, heptane/EtOAc gradient) to give Intermediate 28 (17.231 g, quantitative).

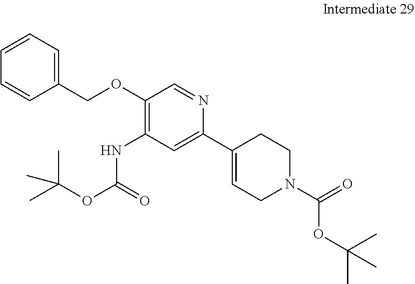 [SMILES: CC(C)(C)OC(=O)Nc1cc(C2=CCN(C(=O)OC(C)(C)C)CC2)ncc1OCc1ccccc1]

To a suspension of Intermediate 28 (6 g, 15.821 mmol), (1-tert-butoxycarbonyl-1,2,3,6-tetrahydropyridin-4-yl)boronic acid pinacol ester (CAS [286961-14-6], 5.919 g, 19.143 mmol, 1.21 eq.), and K₃PO₄ (6.716 g, 31.642 mmol. 2 eq.) in a mixture of 14-dioxane (58 mL) and water (10 mL), under nitrogen atmosphere, Pd(dppf)Cl₂ DCM (CAS [95464-05-4], 648 mg, 0.791 mmol, 0.05 eq.) was added and the mixture was stirred overnight at 100° C. under nitrogen atmosphere. The reaction mixture was partitioned between EtOAc and brine. The layers were separated and the combined organic layers were concentrated. The residue was purified by column chromatography on silica gel (heptane/EtOAc gradient) to give Intermediate 29 (5.73 g, yield: 75%).

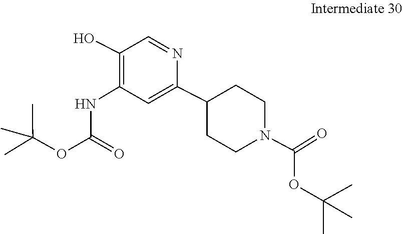 [SMILES: CC(C)(C)OC(=O)Nc1cc(C2CCN(C(=O)OC(C)(C)C)CC2)ncc1O]

Pd/C 10% (500 mg) was added to a solution of Intermediate 29 (5.73 g, 11.898 mmol) in MeOH (150 mL) and THF (50 mL) under nitrogen atmosphere. The mixture was purged with hydrogen and was stirred overnight at room temperature under hydrogen (atmospheric pressure). The mixture was filtered over a pad of celite, and the solvent was removed under reduced pressure to give Intermediate 30 (4.5 g, yield: 96%), used without further purification.

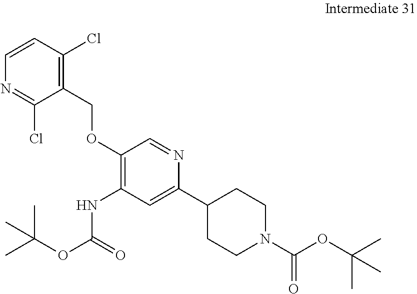 [SMILES: CC(C)(C)OC(=O)Nc1cc(C2CCN(C(=O)OC(C)(C)C)CC2)ncc1OCc1c(Cl)ccnc1Cl]

2,4-Dichloro-3-pyridinemethanol (CAS [945543-24-8], 620 mg, 3.413 mmol), Intermediate 30 (1.343 g, 3.413 mmol, 1 eq.), and triphenylphosphine (1.79 g, 6.826 mmol, 2 eq.) were mixed in dry THF (100 mL) under nitrogen atmosphere. DIAD (1.344 mL, 6.826 mmol, 2 eq.) was then added dropwise and the reaction mixture was stirred at room temperature for 1 h. The solvent was removed under reduced pressure and the reaction mixture was diluted with DCM and SiO₂ was added. Solvents were evaporated and the residue was purified by column flash chromatography (silica; heptane (10% DCM)/EtOAc from 5% EtOAc to 70% EtOAc) to give Intermediate 31 (1.87 g, quantitative).

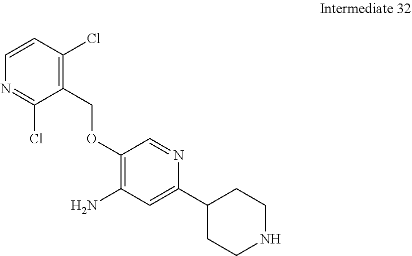 [SMILES: Nc1cc(C2CCNCC2)ncc1OCc1c(Cl)ccnc1Cl]

To a solution of Intermediate 31 (1800 mg, 3.252 mmol) in DCM (18 mL), TFA (12 mL) was added and the reaction mixture was stirred for 3 h at room temperature. The mixture was concentrated under reduced pressure and the residue was diluted with DCM and water and basified with saturated aqueous Na₂CO₃. The organic layer was dried with MgSO₄ and concentrated. The residue was purified by column flash chromatography (SiO₂, DCM/MeOH gradient) to give Intermediate 32 (540 mg, yield: 47%).

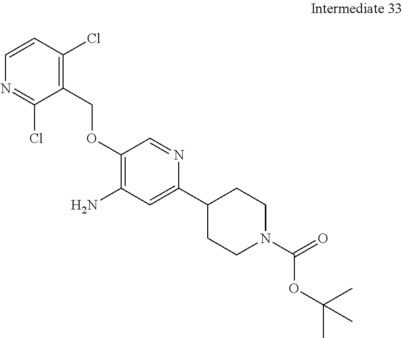 [SMILES: CC(C)(C)OC(=O)N1CCC(c2cc(N)c(OCc3c(Cl)ccnc3Cl)cn2)CC1]

Di-tert-butyl decarbonate (240 mg, 1.101 mmol, 0.8 eq.) was added dropwise to a solution of Intermediate 32 (540 mg, 1.376 mmol) and DIPEA (227 μL, 1.376 mmol, 1 eq.) in DCM at 0° C. The reaction mixture was stirred for 3 h at room temperature. The solvent was evaporated and the residue was purified by flash column chromatography on silica gel (DCM/DCM:MeOHf 9:1 from 100/0 to 0/100) to give Intermediate 33 (480 mg, yield: 77%).

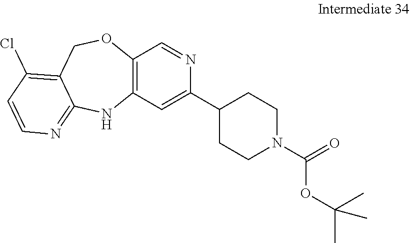 [SMILES: CC(C)(C)OC(=O)N1CCC(c2cc3c(cn2)OCc2c(Cl)ccnc2N3)CC1]

In a sealed tube, a solution of Intermediate 33 (352 mg, 0.776 mmol) and Xantphos (CAS [161265-03-8], 135 mg, 0.233 mmol, 0.3 eq.) in 1,4-dioxane (5 mL) was degassed with nitrogen. Then, Pd₂(dba)₃ (CAS [51364-51-3], 213 mg, 0.233 mmol, 0.3 eq.) and cesium carbonate (1265 mg, 3.882 mmol, 5 eq.) were added. The reaction mixture was degassed again with nitrogen and was stirred at 80° C. for 18 h. The reaction mixture was partitioned between EtOAc and brine. The layers were separated and the combined organic layers were dried (MgSO₄), filtered, and concentrated. The residue was purified by flash column chromatography (silica; heptane/EtOAc from 100/0 to 0/100) to afford Intermediate 34 (152 mg, yield: 47%).

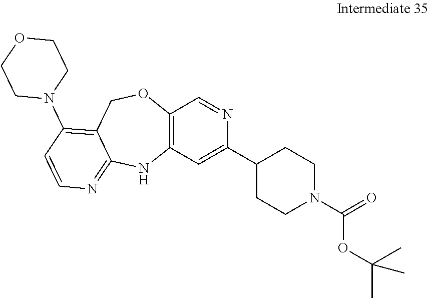 [SMILES: CC(C)(C)OC(=O)N1CCC(c2cc3c(cn2)OCc2c(N4CCOCC4)ccnc2N3)CC1]

In a sealed tube, Intermediate 34 (100 mg, 0.237 mmol) and cesium carbonate (232 mg, 0.712 mmol, 3 eq.) were mixed in dry DMF (1 mL) and the mixture was degassed with nitrogen. Then, morpholine (41 μL, 0.475 mmol, 2 eq.), BINAP (CAS [98327-87-8], 30 mg, 0.047 mmol, 0.2 eq.), and Pd(OAc)₂ (5 mg, 0.024 mmol, 0.1 eq.) were added and the mixture was degassed with nitrogen again. The reaction mixture was stirred at 100° C. for 18 h. The reaction mixture was partitioned between EtOAc and brine. The layers were separated and the combined organic layers were dried (MgSO₄), filtered, and concentrated. The residue was purified by flash column chromatography (silica, DCM/DCM:MeOH 9:1 from 100/0 to 0/100) to give Intermediate 35 (111 mg, quantitative)

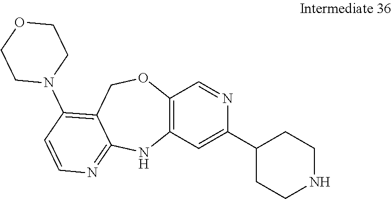 [SMILES: c1cc(N2CCOCC2)c2c(n1)Nc1cc(C3CCNCC3)ncc1OC2]

To a solution of Intermediate 35 (155 mg, 0.332 mmol) in DCM (3 mL), TFA (2 mL) was added and the mixture was stirred for 3 h at room temperature. The mixture was concentrated under reduced pressure and the residue was diluted with DCM and water and basified with saturated aqueous Na₂CO₃. The layers were separated and the organic layer was dried with MgSO₄ and concentrated to give Intermediate 36 (120 mg, yield: 98%), used without further purification.

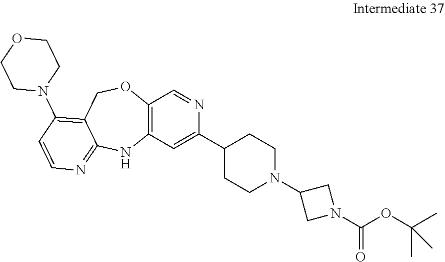 [SMILES: CC(C)(C)OC(=O)N1CC(N2CCC(c3cc4c(cn3)OCc3c(N5CCOCC5)ccnc3N4)CC2)C1]

To a solution of Intermediate 36 (120 mg, 0.327 mmol) and tert-butyl-3-oxoazetidine-1-carboxylate (CAS [398489-26-4], 84 mg, 0.49 mmol, 1.5 eq.) in MeOH (10 mL), AcOH (22 μL, 0.392 mmol, 1.2 eq.) was added and the mixture was stirred for 5 h at room temperature. Then NaBH₃CN (20 mg, 0.327 mmol, 1 eq.) was added and the mixture was stirred at room temperature for 16 h. Additional tert-butyl-3-oxoazetidine-1-carboxylate (84 mg, 0.49 mmol, 1.5 eq.) and NaBH₃CN (20 mg, 0.327 mmol, 1 eq.) were added and the mixture was stirred at room temperature overnight. Aqueous NaHCO₃ was added and the mixture was extracted with EtOAc. The organic layer was dried over MgSO₄, filtered, and evaporated. The residue was purified by flash column chromatography (SiO₂, heptane/EtOAc) to give Intermediate 37 (38 mg, yield: 22%).

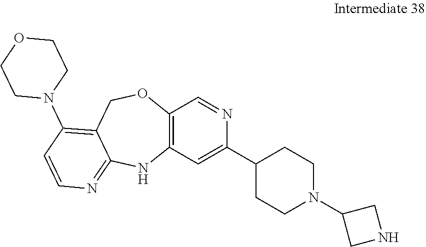 [SMILES: c1cc(N2CCOCC2)c2c(n1)Nc1cc(C3CCN(C4CNC4)CC3)ncc1OC2]

To a solution of Intermediate 37 (38 mg, 0.073 mmol) in DCM (3 mL), TFA (1 mL) was added and the mixture was stirred for 3 h at room temperature. The volatiles were evaporated to give Intermediate 38 (30 mg, quantitative), used without further purification.

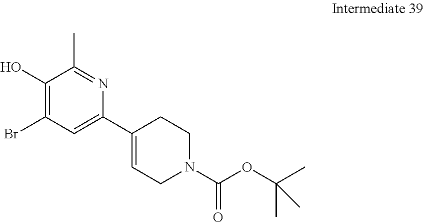 [SMILES: Cc1nc(C2=CCN(C(=O)OC(C)(C)C)CC2)cc(Br)c1O]

Pd(dppf)Cl₂·DCM (CAS [95464-05-4], 307 mg, 0.375 mmol, 0.05 eq.) was added to a suspension of 4,6-dibromo-2-methyl-3-pyridinol (CAS [188923-75-3], 2 g, 7.493 mmol), (1-tert-tutoxycarbonyl-1,2,3,6-tetrahydropyridin-4-yl)boronic acid pinacol ester (CAS [286961-14-6], 1.854 g, 5.994 mmol, 0.8 eq.), and K₃PO₄ (3.181 g, 14.986 mmol, 2 eq.) in a mixture of 1,4-dioxane (48 mL) and water (8 mL), under nitrogen atmosphere, and the mixture was stirred overnight at room temperature under nitrogen atmosphere. The reaction mixture was partitioned between EtOAc and brine. The combined organic layers were concentrated. The residue was purified by column chromatography on silica gel (heptane/EtOAc) to give Intermediate 39 (1.37 g, yield: 50%).

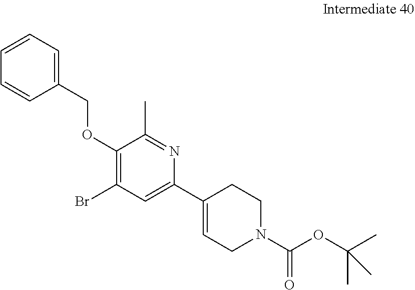 [SMILES: Cc1nc(C2=CCN(C(=O)OC(C)(C)C)CC2)cc(Br)c1OCc1ccccc1]

Benzyl bromide (166 μL, 1.393 mmol, 1.5 eq.) was added to a solution of Intermediate 39 (343 mg, 0.929 mmol), K₂CO₃ (154 mg, 1.115 mmol, 1.2 eq.) in acetone (10 mL), and the mixture was stirred at 50° C. for 15 h. The reaction mixture was partitioned between EtOAc/brine. The combined organic layers were dried (MgSO₄), filtered, and concentrated. The residue was purified by flash column chromatography (silica; heptane/EtOAc from 100/0 to 80/20) to afford Intermediate 40 (389 mg yield: 91%)

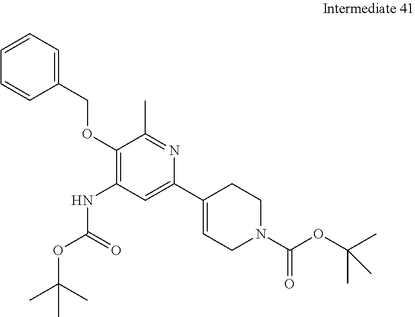 [SMILES: Cc1nc(C2=CCN(C(=O)OC(C)(C)C)CC2)cc(NC(=O)OC(C)(C)C)c1OCc1ccccc1]

Xantphos (CAS [161265-03-8], 1 g, 1.735 mmol, 0.1 eq.) and Pd₂(dba)₃ (CAS [51364-51-3], 1.589 g, 1.735 mmol, 0.1 eq.) were added to a suspension of Intermediate 40 (7.97 g, 17.35 mmol), tert-butyl carbamate (2.642 g, 22.555 mmol, 1.3 eq.), and Cs₂CO₃ (11.3 g, 24.699 mmol, 2 eq.) in toluene (220 mL) under nitrogen atmosphere, and the mixture was stirred for 16 h at 100° C. The reaction mixture was partitioned between EtOAc and brine. The combined organic layers were concentrated and the residue was purified by column chromatography on silica gel (heptane/EtOAc) to give Intermediate 41 (6.69 g, yield: 78%).

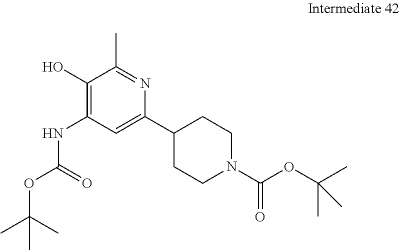 [SMILES: Cc1nc(C2CCN(C(=O)OC(C)(C)C)CC2)cc(NC(=O)OC(C)(C)C)c1O]

Pd/C 10% (560 mg) was added to a solution of Intermediate 41 (6.69 g, 13.499 mmol) in MeOH (350 mL) under nitrogen atmosphere, then hydrogen was bubbled through, and the mixture was stirred overnight at room temperature. The reaction mixture was filtered over a pad of celite and the filtrate was evaporated to give Intermediate 42 (5.4 g, yield: 98%), used without further purification.

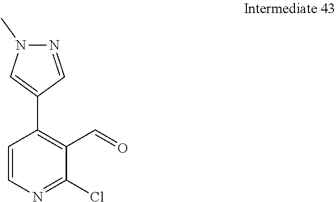

2-Chloro-4-iodo-3-pyridinecarboxaldehyde (CAS [153034-90-3], 2 g, 7.478 mmol), 1-methyl-4-(4,4,5,5-tetramethyl-1,3,2-dioxaborolan-2-yl)-1H-pyrazole (CAS [761446-44-0], 1.556 g, 7.478 mmol, 1 eq.), and Pd(dppf)Cl₂·DCM (CAS [95464-05-4], 183 mg, 0.224 mmol, 0.03 eq.) were placed in a mixture of Na₂CO₃ (1 M in water, 15 mL, 14.956 mmol, 2 eq.) and 1,4-dioxane (30 mL). The reaction mixture was degassed with nitrogen for 15 min. The mixture was then maintained under nitrogen atmosphere and stirred at 45° C. for 4 h. After cooling to room temperature, the reaction mixture was diluted with EtOAc (100 mL). Water (25 mL) was added, followed by brine (50 mL). The organic layer was separated and the aqueous layer was extracted again with EtOAc (100 mL). The combined organic layers were dried over MgSO₄, filtered, and concentrated. The residue was purified by chromatography over silica gel (gradient of EtOAc in heptane from 0 to 75%) to afford Intermediate 43 (1.44 g, yield: 86%) as a light yellow solid.

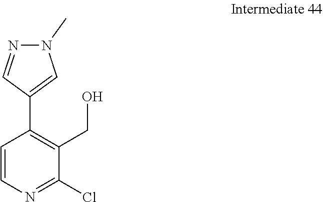

Sodium borohydride (287 mg, 7.58 mmol, 1.2 eq.) was added portionwise to a solution of Intermediate 43 (1.4 g, 6.316 mmol) in MeOH (20 mL) at 5° C. under nitrogen atmosphere. Water and EtOAc were added and the layers were separated. The organic layer was dried over MgSO₄, filtered, and concentrated to give Intermediate 44 (1.39 g, yield: 97%), used without further purification.

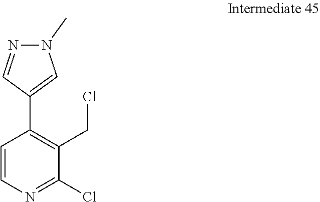

Thionyl chloride (701 μL, 9.322 mmol, 1.5 eq.) was added to a mixture of Intermediate 44 (1.39 g, 6.215 mmol) in DCM (25 mL) at 0° C. under nitrogen atmosphere. The reaction mixture was stirred at room temperature for 3 h. The mixture was concentrated to dryness an water and DCM were added. The layers were separated and the organic layer was dried over MgSO₄, filtered, and the solvent was evaporated to afford Intermediate 45 (1.446 g, yield: 95%) as an oil, used without further purification.

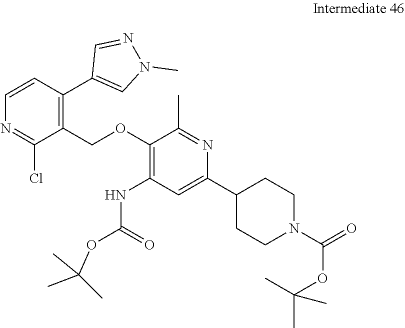 [SMILES: Cc1nc(C2CCN(C(=O)OC(C)(C)C)CC2)cc(NC(=O)OC(C)(C)C)c1OCc1c(-c2cnn(C)c2)ccnc1Cl]

K₂CO₃ (667 mg, 4.824 mmol, 2 eq.) was added to a mixture of Intermediate 45 (759 mg, 3.136 mmol, 1.3 eq.) and Intermediate 42 (983 mg, 2.412 mmol) in DMF (30 mL). The reaction mixture was stirred at 80° C. for 2 h. Water and DCM were added and the layers were separated. The organic layer was dried over MgSO₄, filtered, concentrated, and the residue was purified by column chromatography on silica gel (gradient EtOAc in heptane from 0% to 100%) to afford Intermediate 46 (1112 mg, yield: 71%) as a yellow oil.

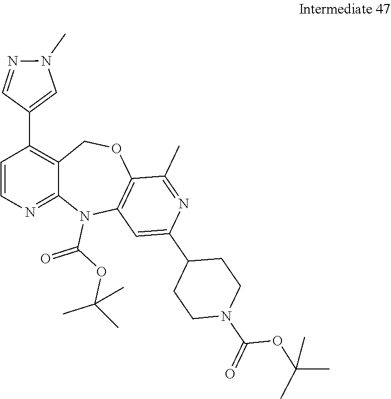 [SMILES: Cc1nc(C2CCN(C(=O)OC(C)(C)C)CC2)cc2c1OCc1c(-c3cnn(C)c3)ccnc1N2C(=O)OC(C)(C)C]

Intermediate 46 (1112 mg, 1.723 mmol) and Cs₂CO₃ (842 mg, 2.584 mmol, 1.5 eq.) were suspended in 1,4-dioxane and degassed with nitrogen for 15 min. Pd₂(dba)₃ (CAS [51364-51-3], 158 mg, 0.172 mmol, 0.1 eq.), Xantphos (CAS [161265-03-8], 199 mg, 0.345 mmol, 0.2 eq.), and Cs₂CO₃ (842 mg, 2.584 mmol, 1.5 eq.) were then added and the resulting mixture was stirred at reflux under nitrogen atmosphere overnight. The reaction mixture was diluted with water (40 mL) and the mixture was extracted with EtOAc (2×50 mL). The combined organic layers were washed with brine, dried over MgSO₄, filtered, concentrated, and the residue was purified by chromatography on silica gel (gradient of MeOH in DCM from 0 to 10%) to afford Intermediate 47 (728 mg, yield: 75%) as a foam.

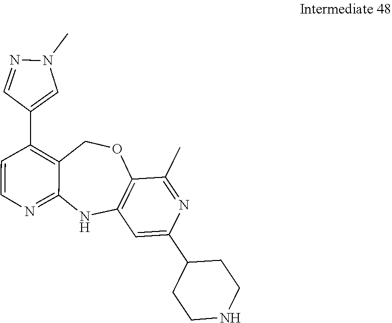 [SMILES: Cc1nc(C2CCNCC2)cc2c1OCc1c(-c3cnn(C)c3)ccnc1N2]

TFA (1 mL, 12.624 mmol, 10 eq.) was added to solution of Intermediate 47 (728 mg, 1.262 mmol) in DCM (25 mL). The reaction mixture was stirred at room temperature overnight. The volatiles were evaporated and the residue was washed with toluene twice and dried to give Intermediate 48 (1195 mg, yield: 98%) as an oil, used without further purification.

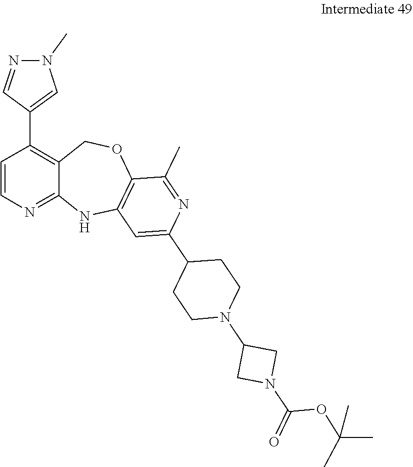 [SMILES: Cc1nc(C2CCN(C3CN(C(=O)OC(C)(C)C)C3)CC2)cc2c1OCc1c(-c3cnn(C)c3)ccnc1N2]

NaBH(OAc)₃ (524 mg, 2.474 mmol, 2 eq.) was added to a solution of Intermediate 48 (1171 mg, 1.237 mmol), Et₃N (688 μL, 4.948 mmol, 4 eq.), and tert-butyl 3-oxoazetidine-1-carboxylate (CAS [398489-26-4], 318 mg, 1.856 mmol, 1.5 eq.) in 1,2-dichloroethane (20 mL). The mixture was stirred at room temperature overnight. NaOH (1 M in water) was added and the mixture was extracted with DCM. The organic layer was dried over MgSO₄, filtered, and concentrated to dryness. The residue was purified by column chromatography on silica gel (gradient of MeOH in DCM from 0% to 20%) to afford Intermediate 49 (421 mg, yield: 63%) as a foam.

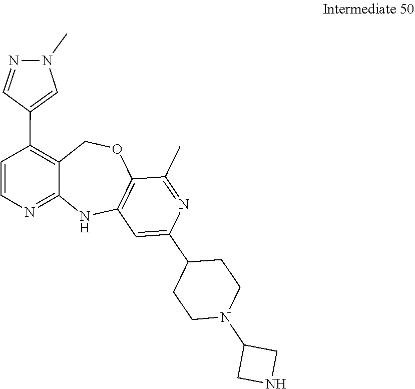 [SMILES: Cc1nc(C2CCN(C3CNC3)CC2)cc2c1OCc1c(-c3cnn(C)c3)ccnc1N2]

TFA (606 μL, 7.919 mmol, 10 eq.) was added to solution of Intermediate 49 (421 mg, 0.792 mmol) in DCM (25 mL). The mixture was stirred overnight at room temperature. The mixture was concentrated to dryness and the residue was washed with toluene twice and dried. Na₂CO₃ (1 M in water) was added and the mixture was extracted with DCM:MeOH (7:1). The organic layer was dried over MgSO₄, filtered, and concentrated to dryness to give Intermediate 50 (341 ng, quantitative) as an oil, used without further purification.

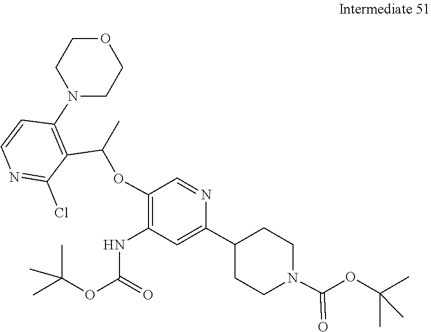 [SMILES: CC(Oc1cnc(C2CCN(C(=O)OC(C)(C)C)CC2)cc1NC(=O)OC(C)(C)C)c1c(N2CCOCC2)ccnc1Cl]

Potassium carbonate (1.238 g, 8.96 mmol, 2 eq.) was added to a solution of Intermediate 30 (1.763 g, 4.48 mmol) and Intermediate 27 (1.17 g, 4.48 mmol, 1 eq.) in dry DMF (60 mL). The reaction mixture was stirred at 60° C. for 16 h. The reaction mixture was diluted with EtOAc and washed with brine. The organic layer was concentrated and the residue was purified by column flash chromatography to give Intermediate 51 (863 mg, yield: 31%).

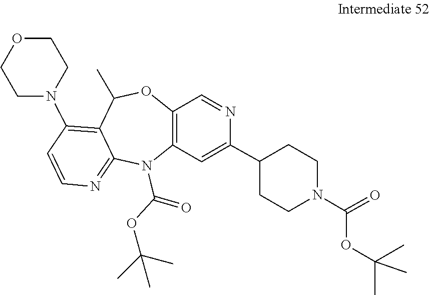 [SMILES: CC1Oc2cnc(C3CCN(C(=O)OC(C)(C)C)CC3)cc2N(C(=O)OC(C)(C)C)c2nccc(N3CCOCC3)c21]

A solution of Intermediate 51 (740 mg, 1.197 mmol) and Cs₂CO₃ (780 mg, 2.394 mmol, 2 eq.) in 1,4-dioxane (40 mL) was degassed with nitrogen. Then, Xantphos (CAS [161265-03-8], 139 mg, 0.239 mmol, 0.2 eq.) and Pd₂(dba)₃ (CAS [51364-51-3], 110 mg, 0.12 mmol, 0.1 eq.) were added. The reaction mixture was degassed again with nitrogen and heated at 100° C. overnight. More Cs₂CO₃ (390 mg, 1.197 mmol, 1 eq.), Xantphos (CAS [161265-03-8], 139 mg, 0.239 mmol, 0.2 eq.) and Pd₂(dba)₃ (CAS [51364-51-3], 110 mg, 0.12 mmol, 0.1 eq.) were added. The reaction mixture was further stirred at 100° C. The reaction mixture was diluted with EtOAc and washed with brine. The organic layer was concentrated and the residue was purified by flash column chromatography (SiO₂, EtOAc/heptane) to give Intermediate 52 (516 mg, yield: 74%)

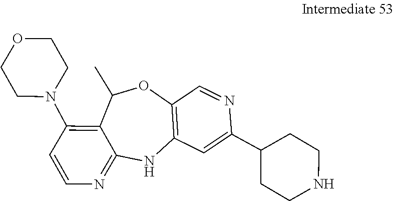 [SMILES: CC1Oc2cnc(C3CCNCC3)cc2Nc2nccc(N3CCOCC3)c21]

TFA (4 mL) was added to a solution of Intermediate 52 (516 mg, 0.887 mmol) in DCM (6 mL), and the mixture was stirred for 3 h at room temperature. The reaction mixture was concentrated under reduced pressure and the residue was diluted with DCM and water and basified with saturated aqueous Na₂CO₃. The organic layer was dried with MgSO₄ and concentrated to give Intermediate 53 (306 mg, yield: 90%), used without further purification.

### Intermediate 54, Intermediate 55 and Intermediate 56

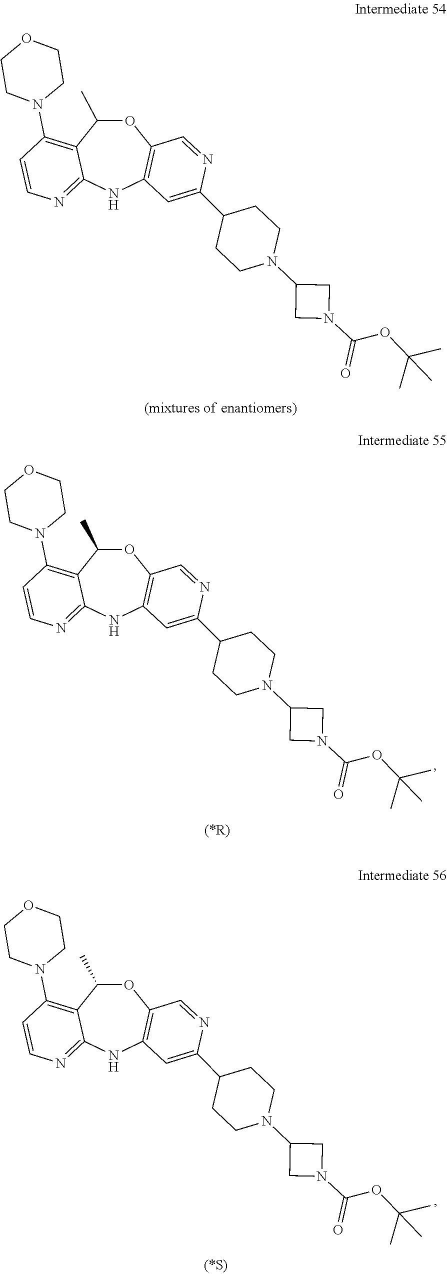 [SMILES: CC1Oc2cnc(C3CCN(C4CN(C(=O)OC(C)(C)C)C4)CC3)cc2Nc2nccc(N3CCOCC3)c21 ; C[C@H]1Oc2cnc(C3CCN(C4CN(C(=O)OC(C)(C)C)C4)CC3)cc2Nc2nccc(N3CCOCC3)c21 ; C[C@@H]1Oc2cnc(C3CCN(C4CN(C(=O)OC(C)(C)C)C4)CC3)cc2Nc2nccc(N3CCOCC3)c21]

Both are pure stereoisomers but with absolute chemistry undetermined Acetic acid (55 μL, 0.963 mmol, 1.2 eq.) was added to a solution of Intermediate 53 (306 mg, 0.802 mmol) and tert-butyl-3-oxoazetidine-1-carboxylate (CAS [398489-26-4], 206 mg, 1.203 mmol, 1.5 eq.) in MeOH (8 mL). The reaction mixture was stirred for 6 h at room temperature. Then NaBH₃CN (76 mg, 1.203 mmol, 1.5 eq.) was added and the mixture was stirred at room temperature for 16 h. More tert-butyl-3-oxoazetidine-1-carboxylate (206 mg, 1.203 mmol, 1.5 eq.) was added and the mixture was stirred for 6 h. Then NaBH₃CN (76 mg, 1.203 mmol, 1.5 eq.) was added and the mixture was stirred at room temperature for 16 h. Again, more tert-butyl-3-oxoazetidine-1-carboxylate (206 mg, 1.203 mmol, 1.5 eq.) was added and the mixture was stirred for 6 h. Then NaBH₃CN (76 mg, 1.203 mmol, 1.5 eq.) was added and the mixture was stirred at room temperature for 16 h. Aqueous NaHCO₃ was added to the reaction mixture and it was extracted with EtOAc. The organic layer was dried over MgSO₄, filtered, and evaporated. The residue was purified by flash column chromatography (gradient DCM-MeOH) to give Intermediate 54 (360 mg, yield: 84%). Intermediate 54 was separated into its enantiomers by normal phase chiral chromatography (Phenomenex Lux Amylose-1 250×30 mm 5 μm; gradient from 50% [heptane+0.1% DEA]−50% [iPrOH)+0.1% DEA] to 100% [iPrOH+0.1% DEA]) to afford Intermediate 55 (140 mg, yield: 39%) and Intermediate 56 (133 mg, yield: 37%).

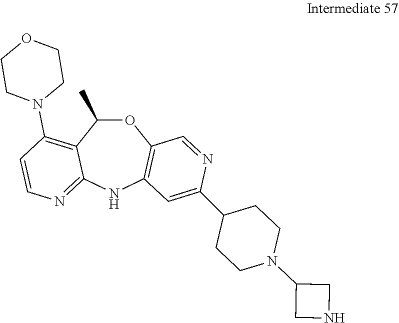 [SMILES: C[C@H]1Oc2cnc(C3CCN(C4CNC4)CC3)cc2Nc2nccc(N3CCOCC3)c21]

(*R), pure stereoisomer but absolute stereochemistry undetermined TFA (4 mL) was added to a solution of Intermediate 55 (140 mg, 0.261 mmol) in DCM (6 mL) and the mixture was stirred for 3 h at room temperature. The volatiles were evaporated to afford Intermediate 57 (64 mg, yield: 56%).

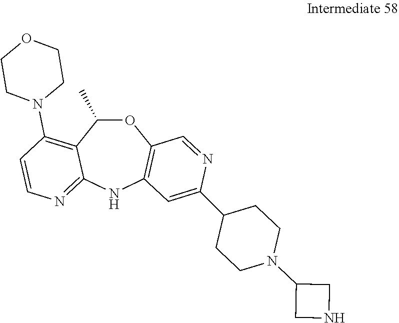 [SMILES: C[C@@H]1Oc2cnc(C3CCN(C4CNC4)CC3)cc2Nc2nccc(N3CCOCC3)c21]

(*S), pure stereoisomer but absolute stereochemistry undetermined Intermediate 58 was prepared according to a procedure similar to Intermediate 57, starting from Intermediate 56 instead of Intermediate 55.

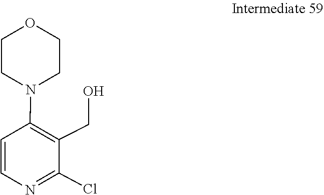 [SMILES: OCc1c(N2CCOCC2)ccnc1Cl]

2-Chloro-4-(4-morpholinyl)-3-pyridinecarboxaldehyde (CAS [877054-85-8], 9.71 g, 42.839 mmol) was dissolved in MeOH (400 mL) and the solution was cooled to 0° C. under nitrogen atmosphere. Sodium borohydride (1.621 g, 42.839 mmol, 1 eq.) was added and the reaction mixture was stirred at 0° C. for 25 min. Water (200 mL) was added carefully and the mixture was extracted with DCM (600 mL). The aqueous layer was extracted with DCM (5×200 mL). The combined organic layers were dried over MgSO₄, filtered, and evaporated in vacuo. The residue was purified by flash column chromatography (silica; EtOAc in heptane 0/100 to 100/0) to give Intermediate 59 (9.796 g, yield: 95%).

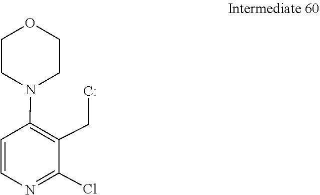

Thionyl chloride (4.57 mL, 61.115 mmol, 1.5 eq.) was added to a mixture of Intermediate 59 (9.317 g, 40.743 mmol) in DCM (160 mL) cooled to 0° C., under nitrogen atmosphere. The reaction mixture was stirred at room temperature for 1.5 h. Water (75 mL) was added and the layers were separated. The organic layer was dried over MgSO₄, filtered, and evaporated in vacuo to give Intermediate 60 (10.07 g, quantitative) as a yellow oil, used without further purification.

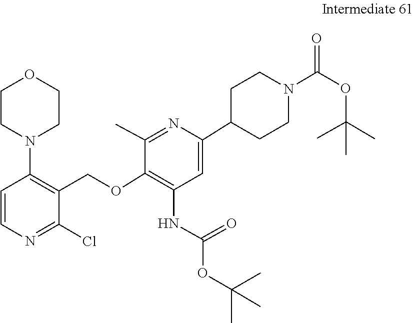 [SMILES: Cc1nc(C2CCN(C(=O)OC(C)(C)C)CC2)cc(NC(=O)OC(C)(C)C)c1OCc1c(N2CCOCC2)ccnc1Cl]

Intermediate 42 (800 mg, 1.963 mmol, 1.05 eq.) was added to a mixture of Intermediate 60 (462 mg, 1.87 mmol) and K₂CO₃ (517 mg, 3.739 mmol, 2 eq.) in DMF (30 mL). The reaction mixture was stirred at 80° C. for 2 h. Water and DCM were added and the layers were separated. The organic layer was dried over MgSO₄, filtered, and concentrated. The residue was purified by column chromatography on silica gel (gradient of EtOAc in heptane from 0% to 100%) to afford Intermediate 61 (1074 mg, yield: 83%) as a yellow oil.

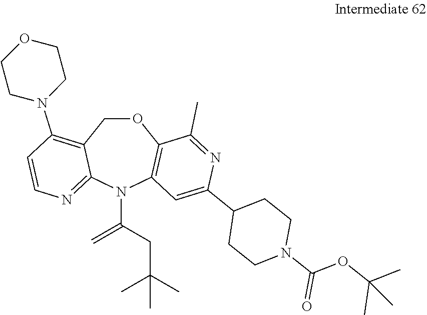 [SMILES: C=C(CC(C)(C)C)N1c2cc(C3CCN(C(=O)OC(C)(C)C)CC3)nc(C)c2OCc2c(N3CCOCC3)ccnc21]

Intermediate 61 (1.074 g, 1.737 mmol) and Cs₂CO₃ (849 mg, 2.606 mmol, 1.5 eq.) were suspended in 1,4-dioxane (20 mL) and the mixture was degassed with nitrogen for 15 min. Pd₂(dba)₃ (CAS [51364-51-3], 79 mg, 0.087 mmol, 0.05 eq.) and Xantphos (CAS [161265-03-8], 101 mg, 0.174 mmol, 0.1 eq.) were then added and the resulting mixture was refluxed overnight under nitrogen atmosphere. The reaction mixture was diluted with water (40 mL) and the mixture was extracted with EtOAc (2×50 mL). The combined organic layers were washed with brine, dried over MgSO₄, filtered, and evaporated. The residue was purified by column chromatography (silica gel, DCM/MeOH/NH₃ 9/0.9/0.1 in DCM from 0% to 40%) to afford Intermediate 62 (765 mg, yield: 75%) as an oil.

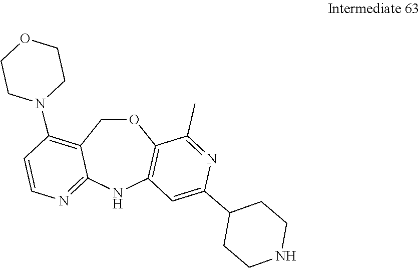 [SMILES: Cc1nc(C2CCNCC2)cc2c1OCc1c(N3CCOCC3)ccnc1N2]

TFA (1 mL, 13.151 mmol, 10 eq.) was added to solution of Intermediate 62 (765 mg, 1.315 mmol) in DCM (25 mL). The reaction mixture was stirred overnight at room temperature. The volatiles were evaporated and the residue was dissolved in DCM. The solution was washed with a mixture of aqueous Na₂CO₃ (1 M, 10 mL) and brine (5 mL). The organic layer was dried over MgSO₄, filtered, and evaporated. The residue was purified by column chromatography over silica gel (DCM/MeOH/NH₃ 9/0.9/0.1 in DCM from 0 to 100%) to give Intermediate 63 (313 mg, yield: 62%) as a yellow oil.

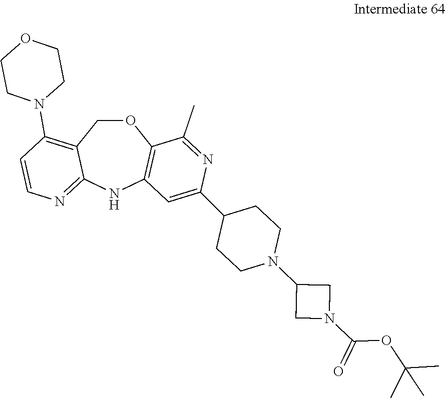 [SMILES: Cc1nc(C2CCN(C3CN(C(=O)OC(C)(C)C)C3)CC2)cc2c1OCc1c(N3CCOCC3)ccnc1N2]

Intermediate 63 (313 mg, 0.821 mmol) was dissolved in DCE (25 mL). tert-butyl 3-oxoazetidine-1-carboxylate (CAS [398489-26-4], 281 mg, 1.641 mmol, 2 eq.) and AcOH (47 μL, 0.821 mmol, 1 eq.) were added and the mixture was stirred at room temperature for 30 min. NaBH(OAc)₃ (261 mg, 1.231 mmol, 1.5 eq.) was then added portionwise and the mixture was stirred at room temperature for 3 h. The mixture was diluted with DCM (50 mL) and washed with Na₂CO₃ (1 M in water, 20 mL). The aqueous layers were extracted once more with DCM (50 mL). The combined organic layers were dried over MgSO₄, filtered, and evaporated. The residue was purified by column chromatography over silica gel (DCM/MeOH/NH₃ 9/0.9/0.1 in DCM from 0 to 85%) to give Intermediate 64 (361 mg, yield: 81%) as a white foam.

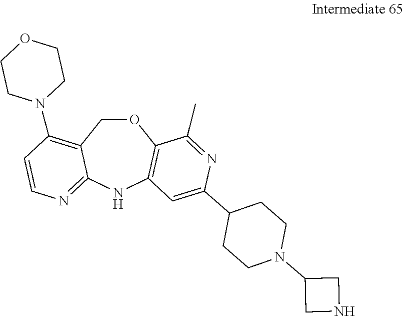 [SMILES: Cc1nc(C2CCN(C3CNC3)CC2)cc2c1OCc1c(N3CCOCC3)ccnc1N2]

TFA (515 μL, 6.727 mmol, 10 eq.) was added to a solution of Intermediate 64 (361 mg, 0.673 mmol) in DCM (15 mL). The mixture was stirred overnight at room temperature. The volatiles were evaporated and the residue was dissolved in DCM and washed with a mixture of Na₂CO₃ (I M in water, 10 mL) and brine (5 mL). The organic layer was dried over MgSO₄, filtered, and evaporated. The residue was purified by column chromatography over silica gel (DCM/MeOH/NH₃ 9/0.9/0.1 in DCM from 0 to 100%) to give Intermediate 65 (216 mg, yield: 73%) as a white solid.

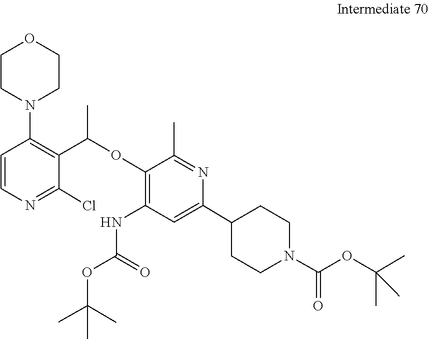 [SMILES: Cc1nc(C2CCN(C(=O)OC(C)(C)C)CC2)cc(NC(=O)OC(C)(C)C)c1OC(C)c1c(N2CCOCC2)ccnc1Cl]

K₂CO₃ (1.843 g, 13.332 mmol, 3 eq.) was added to a suspension of Intermediate 42 (1.811 g, 4.444 mmol) in DMF (18 mL). The reaction mixture was stirred at room temperature, then Intermediate 27 (1.393 g, 5.333 mmol, 1.2 eq.) was added in four portions over 4 h. The reaction mixture was stirred at room temperature for 20 h. To push the reaction to completion, more K₂CO₃ (614 mg, 4.444 mmol, 1 eq.) was added, followed by Intermediate 27 (928 mg, 3.555 mmol, 0.8 eq.) in 4 portions over 4 h. The reaction mixture was further stirred at room temperature for 16 h. The reaction mixture was partitioned between EtOAc and brine. The combined organic layers were dried (MgSO₄), filtered, and concentrated. The residue was purified twice by flash column chromatography (silica, heptane/EtOAc from 100/0 to 0/100) to afford Intermediate 70 (2.206 g, yield: 62%) as a yellow foam.

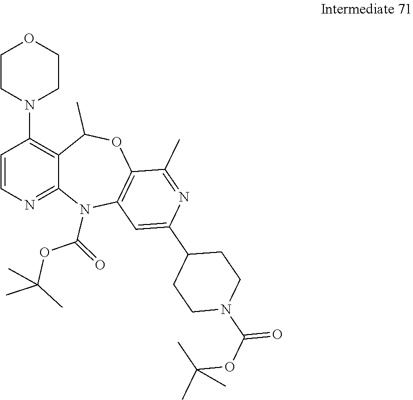 [SMILES: Cc1nc(C2CCN(C(=O)OC(C)(C)C)CC2)cc2c1OC(C)c1c(N3CCOCC3)ccnc1N2C(=O)OC(C)(C)C]

Intermediate 70 (4.624 g, 7.314 mmol) and Cs₂CO₃ (3.575 g, 10.971 mmol, 1.5 eq.) were suspended in toluene (80 mL) and the mixture was degassed with nitrogen for 15 min. Palladium(II) acetate (CAS [3375-31-3], 86 mg, 0.1 eq.) and Xantphos (CAS [161265-03-8], 423 mg, 0.731 mmol, 0.1 eq.) were then added and the resulting mixture was stirred overnight at 120° C. under nitrogen atmosphere. After cooling, the reaction mixture was diluted with water (100 mL) and EtOAc (250 mL). The organic layer was separated, dried over MgSO₄, filtered, and evaporated. The residue was purified by column chromatography over silica gel (gradient DCM/MeOH (9:1) in DCM from 0% to 50%) to give Intermediate 71 (3.92 g, yield: 82%) as an oil.

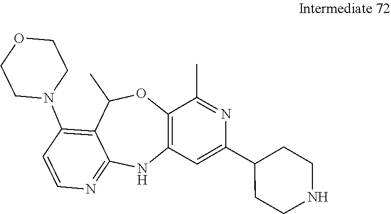 [SMILES: Cc1nc(C2CCNCC2)cc2c1OC(C)c1c(N3CCOCC3)ccnc1N2]

TFA (12.5 mL) was added to a solution of Intermediate 71 (1.865 g, 3.131 mmol) in DCM (19 mL) at room temperature. The reaction mixture was stirred at room temperature for 3 h. The volatiles were evaporated and the residue was partitioned between DCM and saturated aqueous K₂CO₃. The combined organic layers were dried (MgSO₄), filtered, and concentrated to afford Intermediate 72 (1.238 g, quantitative), used without further purification.

### Intermediate 73, Intermediate 74, and Intermediate 75

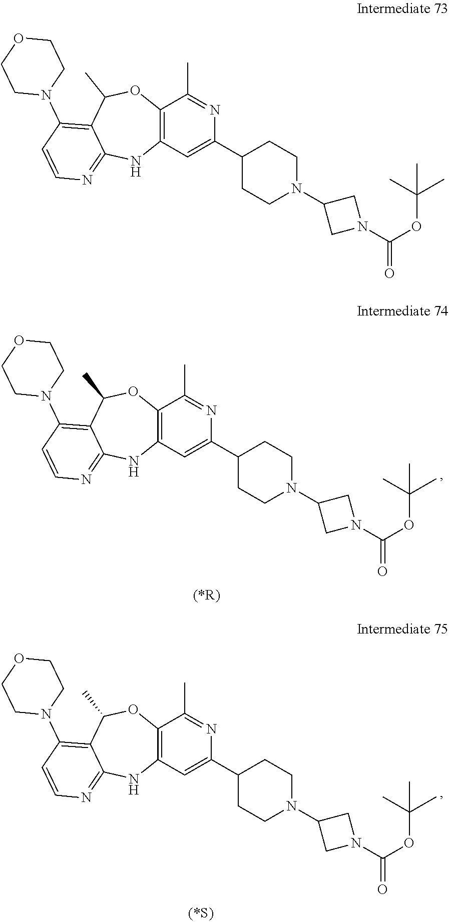 [SMILES: Cc1nc(C2CCN(C3CN(C(=O)OC(C)(C)C)C3)CC2)cc2c1OC(C)c1c(N3CCOCC3)ccnc1N2 ; Cc1nc(C2CCN(C3CN(C(=O)OC(C)(C)C)C3)CC2)cc2c1O[C@H](C)c1c(N3CCOCC3)ccnc1N2 ; Cc1nc(C2CCN(C3CN(C(=O)OC(C)(C)C)C3)CC2)cc2c1O[C@@H](C)c1c(N3CCOCC3)ccnc1N2] [0390] Both are pure enantiomers but absolute stereochemistry undetermined Acetic acid (206 μL, 3.605 mmol, 1.2 eq.) was added to a solution of Intermediate 72 (1.188 g, 3.004 mmol) and tert-butyl-3-oxoazetidine-1-carboxylate (CAS [398489-26-4], 771 mg, 4.506 mmol, 1.5 eq.) in MeOH (20 mL), and the mixture was stirred at room temperature for 4 h. NaBH₃CN (189 mg, 3.004 mmol, 1 eq.) was added and the mixture was stirred at room temperature for 20 h. More tert-butyl-3-oxoazetidine-1-carboxylate (771 mg, 4.506 mmol, 1.5 eq.) was added and the mixture was stirred for 3 h. NaBH₃CN (189 mg, 3.004 mmol, 1 eq.) was added and the mixture was stirred at room temperature for 2 days. The reaction mixture was partitioned between EtOAc and saturated aqueous NaHCO₃. The combined organic layers were dried (MgSO₄), filtered, and evaporated. The residue was purified by flash column chromatography (silica, DCM/DCM:MeOH (9:1) from 100/0 to 30/70) to afford Intermediate 73 (1.123 g, yield: 64%) as a white solid. Intermediate 73 was separated into its enantiomers by chiral column chromatography (AMYLOSE_1 Q_M6; [heptane-(iPrOH-EtOH 9:1)]+0.1% Et₂NH) to afford Intermediate 74 (430 mg, yield: 38%) and Intermediate 75 (423 mg, yield: 37%).

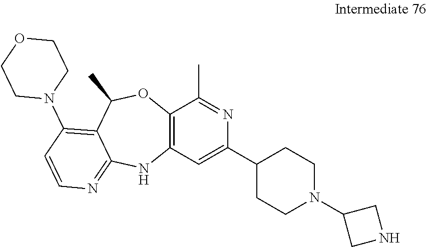 [SMILES: Cc1nc(C2CCN(C3CNC3)CC2)cc2c1O[C@H](C)c1c(N3CCOCC3)ccnc1N2]

(*R), Pure enantiomer but absolute stereochemistry undetermined TFA (4 mL) was added to a solution of Intermediate 74 (430 mg, 0.781 mmol) in DCM (6 mL) and the mixture was stirred for 3 h at room temperature. The volatiles were evaporated to afford Intermediate 76 (351 mg, quantitative).

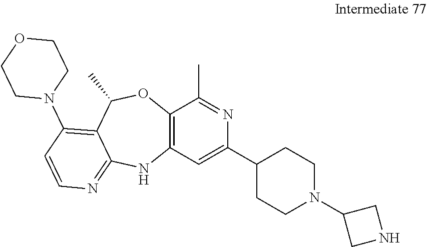 [SMILES: Cc1nc(C2CCN(C3CNC3)CC2)cc2c1O[C@@H](C)c1c(N3CCOCC3)ccnc1N2]

(*S), Pure enantiomer but absolute stereochemistry undetermined Intermediate 77 was prepared by a procedure analogous to Intermediate 76, starting from Intermediate 75 instead of Intermediate 74.

### Intermediate 78

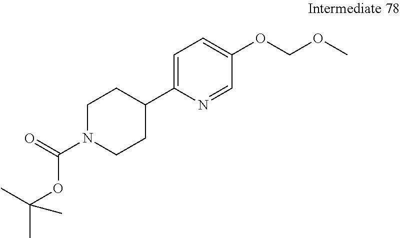 [SMILES: COCOc1ccc(C2CCN(C(=O)OC(C)(C)C)CC2)nc1]

2-Chloro-5-(methoxymethoxy)pyridine (CAS [877133-56-7], 7.936 g, 45.714 mmol), Pd(dppf)Cl₂·DCM (CAS [95464-05-4], 1.867 g, 2.286 mmol, 0.05 eq.), and CuI (871 mg, 4.571 mmol, 0.1 eq.) were dissolved in DMA (81 mL) under nitrogen atmosphere. A solution of [1-(tert-butoxycarbonyl)piperidin-4-yl]zinc iodide (CAS [807618-13-9], 24.1 g, 64 mmol, 1.4 eq.) in DMA (100 mL) was added via syringe and the resulting mixture was stirred at 80° C. for 1 h under nitrogen atmosphere. After cooling, the reaction mixture was diluted with EtOAc (100 mL). Saturated aqueous NH₄Cl (25 mL) was added while stirring, followed by water (50 mL). The organic layer was separated and washed with brine (50 mL), dried over MgSO₄, filtered, and concentrated. The residue was purified by column chromatography (silica gel, gradient of EtOAc in heptane from 0 to 50%) to give Intermediate 78 (10.98 g, yield: 71%) as a gummy residue that solidified upon standing.

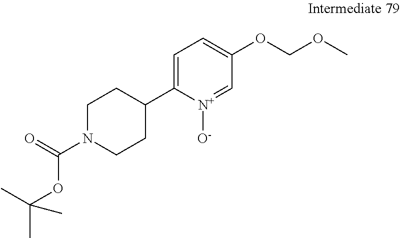 [SMILES: COCOc1ccc(C2CCN(C(=O)OC(C)(C)C)CC2)[n+]([O-])c1]

mCPBA (11.105 g, 49.553 mmol, 1.5 eq.) was dissolved in CHCl₃ (100 mL) and dried over MgSO₄. The solution was filtered and the filtrate was added dropwise to a solution of Intermediate 78 (10.98 g. 33.035 mmol) in CHCl₃ (80 mL). The reaction mixture was stirred at room temperature overnight. Na₂CO₃ (1 M in water) was added and the mixture was extracted with DCM/MeOH (9:1). The organic layer was dried over MgSO₄, filtered, and evaporated. The residue was purified by column chromatography over silica gel (gradient of EtOAc in heptane from 0% to 60%) to give Intermediate 79 (5.202 g, yield: 46%) as an oil.

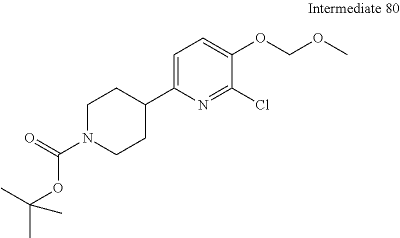 [SMILES: COCOc1ccc(C2CCN(C(=O)OC(C)(C)C)CC2)nc1Cl]

Intermediate 79 (5.202 g, 15.372 mmol) and Et₃N (21.4 mL, 153.724 mmol, 10 eq.) were dissolved in DCE (40 mL). POCl₃ (1.43 mL, 15.372 mmol, 1 eq.) was then added and the resulting mixture was refluxed under nitrogen atmosphere for 20 min. Na₂CO₃ (1 M in water) was added and the mixture was extracted with DCM/MeOH (9:1). The organic layer was dried over MgSO₄, filtered, and evaporated. The residue was purified by column chromatography over silica gel (gradient of EtOAc in heptane from 0% to 60%)) to afford Intermediate 80 (1450 mg, yield: 26%) as an oil.

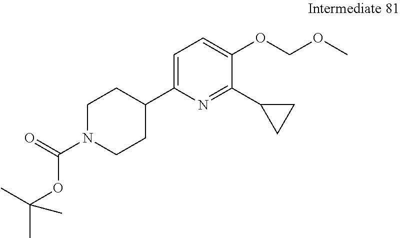 [SMILES: COCOc1ccc(C2CCN(C(=O)OC(C)(C)C)CC2)nc1C1CC1]

Intermediate 80 (1401 mg, 3.926 mmol), cyclopropylboronic acid (CAS [411235-57-9], 1349 mg, 15.704 mmol, 4 eq.) and Pd(dppf)Cl₂ (CAS [95464-05-4], 160 mg, 0.196 mmol, 0.05 eq.) were dissolved in water (5 mL) and 1,4-dioxane (30 mL) and the mixture was degassed with nitrogen for 15 min. K₃PO₄ (2.5 g, 11.778 mmol, 3 eq.) was then added and the reaction mixture was stirred at 100° C. under nitrogen atmosphere for 20 h. After cooling, the reaction mixture was diluted with DCM and washed with a Na₂CO₃ (1 M in water, 10 mL). The organic layer was dried over MgSO₄, filtered, and evaporated. The residue was purified by column chromatography over silica gel (EtOAc in heptane from 0/100 to 80/20) to give Intermediate 81 (902 mg, yield: 63%) as an oil.

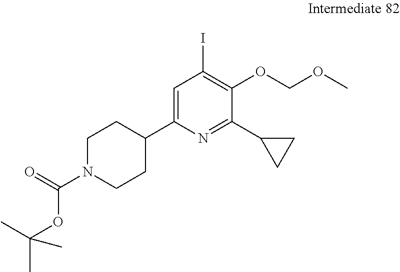 [SMILES: COCOc1c(I)cc(C2CCN(C(=O)OC(C)(C)C)CC2)nc1C1CC1]

Intermediate 81 (902 mg, 2.489 mmol) was dissolved in dry THF (25 mL) and the solution was cooled to −78° C. under nitrogen atmosphere. n-BuLi (1.6 M in THF, 1.866 mL, 1.2 eq.) was added dropwise over 10-15 min and stirring was continued for 15 min. A solution of iodine (758 mg, 2.986 mmol, 1.2 eq.) in dry THF (5 mL) was then added dropwise over 15 min. Stirring was continued for 1 h. The reaction was quenched by adding water (25 mL). EtOAc (50 mL) and saturated aqueous Na₂S₂O₃ (15 mL) were added. The organic layer was separated, dried over MgSO₄, filtered, and evaporated. The residue was purified by column chromatography over silica gel (gradient of EtOAc in heptane from 0/100 to 50/50) to afford Intermediate 82 (985 mg, yield: 81%) as an oil.

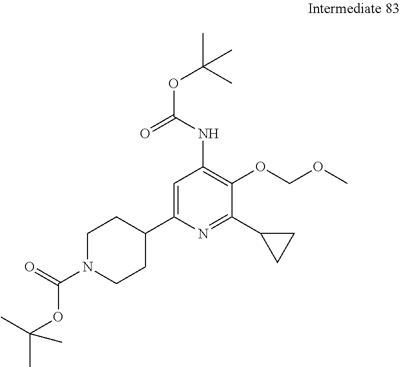 [SMILES: COCOc1c(NC(=O)OC(C)(C)C)cc(C2CCN(C(=O)OC(C)(C)C)CC2)nc1C1CC1]

Intermediate 82 (985 mg, 2.017 mmol), tbutyl carbamate (260 mg, 2.219 mmol, 1.1 eq.), Pd₂(dba)₃ (CAS [51364-51-3], 55 mg, 0.06 mmol, 0.03 eq.), Xantphos (CAS [161265-03-8], 70 mg, 0.121 mmol, 0.06 eq.), and Cs₂CO₃ (1314 mg, 4.034 mmol, 2 eq.) were suspended in toluene (30 mL) and the mixture was degassed by bubbling nitrogen for 15 min. The reaction mixture was stirred at 100° C. under nitrogen atmosphere for 3 h. After cooling, the mixture was concentrated to half its volume. Water (15 mL) was added and the mixture was extracted with EtOAc (2×30 mL). The combined organic layers were washed with brine, dried over MgSO₄, filtered, and concentrated. The residue was purified by column chromatography over silica gel (gradient of EtOAc in heptane from 0 to 50%) to afford Intermediate 83 (789 mg, yield: 81%) as an oil.

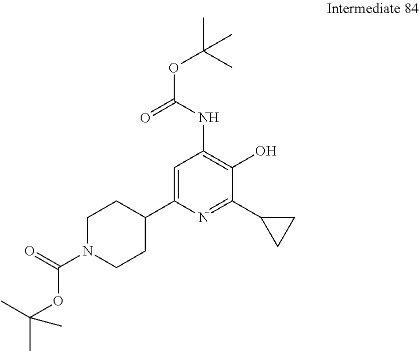 [SMILES: CC(C)(C)OC(=O)Nc1cc(C2CCN(C(=O)OC(C)(C)C)CC2)nc(C2CC2)c1O]

HCl (37% in water, 165 μL, 1.983 mmol, 1.2 eq.) was added to a solution of Intermediate 83 (789 mg, 1.653 mmol) in iPrOH (15 mL). The reaction mixture was stirred at room temperature for 5 days. Water and saturated aqueous NaHCO₃ were added until pH=7. The mixture was extracted with DCM and the organic layer was dried over MgSO₄, filtered, and evaporated. The residue was purified by column chromatography over silica gel (gradient of EtOAc in heptane from 0% to 100%) to afford Intermediate 84 (432 rmg, yield: 60%) as an oil.

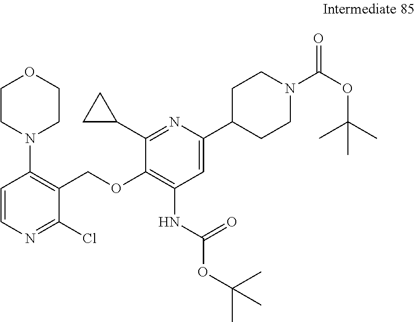 [SMILES: CC(C)(C)OC(=O)Nc1cc(C2CCN(C(=O)OC(C)(C)C)CC2)nc(C2CC2)c1OCc1c(N2CCOCC2)ccnc1Cl]

Intermediate 84 (432 mg, 0.996 mmol) was added to a mixture of Intermediate 60 (271 mg, 1.096 mmol, 1.1 eq.) and K₂CO₃ (275 mg, 1.993 mmol, 2 eq.) in DMF (15 mL). The reaction mixture was stirred at room temperature for 4 days. Water and DCM were added and the layers were separated. The organic layer was dried over MgSO₄, filtered, concentrated and purified by column chromatography over silica gel (gradient of EtOAc in heptane from 0% to 100% to give Intermediate 85 (471 mg, yield: 70%) as an oil.

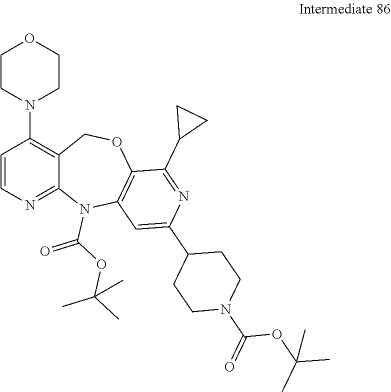 [SMILES: CC(C)(C)OC(=O)N1CCC(c2cc3c(c(C4CC4)n2)OCc2c(N4CCOCC4)ccnc2N3C(=O)OC(C)(C)C)CC1]

Intermediate 85 (540 mg, 0.838 mmol) and Cs₂CO₃ (410 mg, 1.257 mmol, 1.5 eq.) were suspended in toluene (40 mL) and the mixture was degassed with nitrogen for 15 min. Pd₂(dba)₃ (CAS [51364-51-3], 38 mg, 0.042 mmol, 0.05 eq.) and Xantphos (CAS [16265-03-8], 48 mg, 0.084 mmol, 0.1 eq.) were then added and the resulting mixture was refluxed under nitrogen atmosphere overnight. The reaction mixture was diluted with water (40 mL) and extracted with EtOAc (2×50 mL). The combined organic layers were washed with brine, dried over MgSO₄, filtered, concentrated and purified by column chromatography over silica gel (DCM/MeOH/NH₃ 9/0.9/0.1 in DCM from 0 to 40%) to afford Intermediate 86 (301 mg, yield: 56%) as a brown oil.

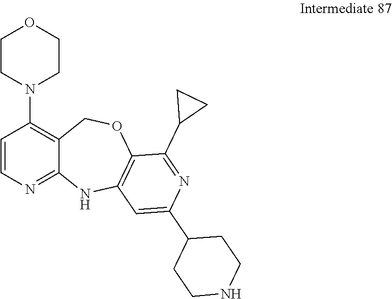 [SMILES: c1cc(N2CCOCC2)c2c(n1)Nc1cc(C3CCNCC3)nc(C3CC3)c1OC2]

TFA (379 μL, 4.953 mmol, 10 eq.) was added to solution of Intermediate 86 (301 mg, 0.495 mmol) in DCM (20 mL). The mixture was stirred at room temperature overnight. The volatiles were evaporated and the residue was dissolved in DCM and washed with Na₂CO₃ (1 M in water, 10 mL) and brine (5 mL). The organic layer was dried over MgSO₄, filtered, and concentrated afford Intermediate 87 (202 mg, quantitative), used without further purification.

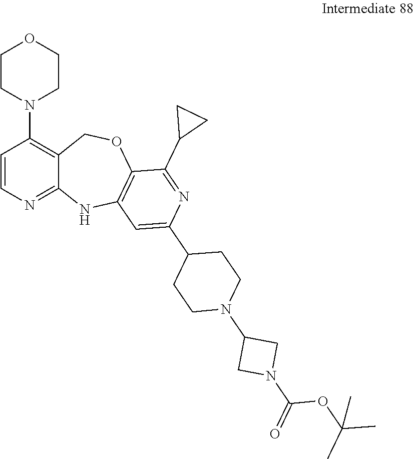 [SMILES: CC(C)(C)OC(=O)N1CC(N2CCC(c3cc4c(c(C5CC5)n3)OCc3c(N5CCOCC5)ccnc3N4)CC2)C1]

NaBH(OAc)₃ (314 mg, 1.48 mol, 3 eq.) was added to a solution of Intermediate 87 (201 mg, 0.493 mmol), Et₃N (274 μL, 1.973 mmol, 4 eq.), and 1-Boc-3-azetidinone (CAS [398489-26-4], 253 mg, 1.48 mmol, 3 eq.) in DCE (30 mL). The mixture was stirred at room temperature overnight. Na₂CO₃ (1 M in water) was added and the mixture was extracted with DCM. The organic layer was dried over MgSO₄, filtered, and evaporated. The residue was purified by column chromatography over silica gel (MeOH in DCM from 0% to 20%) to obtain Intermediate 88 (205 mg, yield: 66%) as a foam.

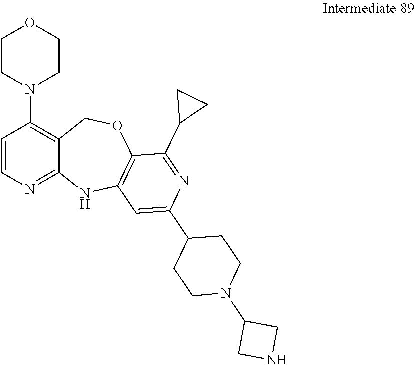 [SMILES: c1cc(N2CCOCC2)c2c(n1)Nc1cc(C3CCN(C4CNC4)CC3)nc(C3CC3)c1OC2]

TFA (279 μL, 3.643 mmol, 10 eq.) was added to solution of Intermediate 88 (205 mg, 0.364 mmol) in DCM (10 mL). The mixture was stirred at room temperature overnight. The volatiles were evaporated and the residue was dissolved in DCM and washed with Na₂CO₃ (1 M in water, 5 mL). The organic layer was dried over MgSO₄, filtered, and evaporated to afford Intermediate 89 (158 mg, yield: 94%), used without further purification.

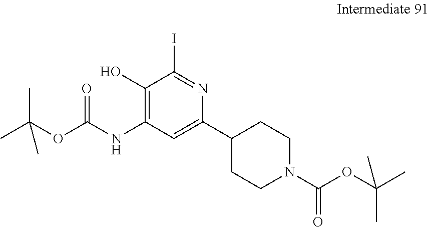 [SMILES: CC(C)(C)OC(=O)Nc1cc(C2CCN(C(=O)OC(C)(C)C)CC2)nc(I)c1O]

In a sealed tube, Intermediate 30 (2.725 g, 6.925 mmol) was dissolved in DMSO (21 mL). K₂CO₃ (2.873 g, 20.776 mmol, 3 eq.) followed by water (73 mL) were added. Finally, iodine (2.109 g, 8.311 mmol, 1.2 eq.) was added and the mixture was stirred at room temperature overnight. The reaction mixture was diluted with brine and extracted with EtOAc. The organic layer was washed with brine (5 x). The solvent was evaporated to give Intermediate 91 (3.075 g, yield: 85%), used without further purification.

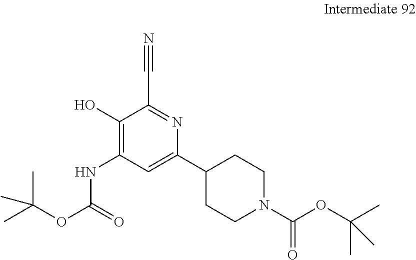 [SMILES: CC(C)(C)OC(=O)Nc1cc(C2CCN(C(=O)OC(C)(C)C)CC2)nc(C#N)c1O]

A mixture of Intermediate 91 (3.075 g, 5.921 mmol) and copper(I) cyanide (1.591 g, 17.762 mmol, 3 eq.) in pyridine (22 mL) was stirred at 80° C. overnight. The reaction mixture was diluted with dilute AcOH and extracted with EtOAc. The organic layer was evaporated and the residue was purified by reverse phase chromatography (Phenomenex Gemini C18 30×100 mm 5 μm; gradient from 70% [25 mM NH₄HCO₃]−30% ACN to 27% [25 mM NH₄HCO₃]−73% ACN) to give Intermediate 92 (1.382 g, yield: 56%).

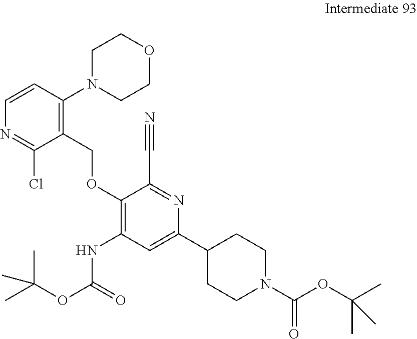 [SMILES: CC(C)(C)OC(=O)Nc1cc(C2CCN(C(=O)OC(C)(C)C)CC2)nc(C#N)c1OCc1c(N2CCOCC2)ccnc1Cl]

K₂CO₃ (685 mg, 4.954 mmol, 3 eq.) was added to a solution of Intermediate 92 (691 mg, 1.651 mmol) and Intermediate 60 (530 mg, 2.147 mmol, 1.3 eq.) in dry DMF (16 mL). The reaction mixture was stirred at room temperature for 15 h. More Intermediate 60 (204 mg, 0.826 mmol, 0.5 eq.) was added and the reaction mixture was stirred at room temperature for 15 h. Again, more Intermediate 60 (204 mg, 0.826 mmol, 0.5 eq.) was added and the reaction mixture was stirred at room temperature for 6 h. The mixture was diluted with brine and extracted with EtOAc. The organic layer was washed with brine (5×), dried, and concentrated. The residue was purified by column chromatography on silica gel (hexane/EtOAc gradient followed by DCM/MeOH gradient) to give Intermediate 93 (226 mg, yield: 22%).

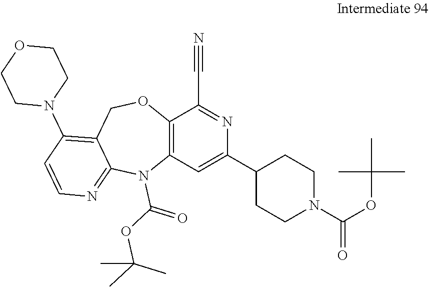 [SMILES: CC(C)(C)OC(=O)N1CCC(c2cc3c(c(C#N)n2)OCc2c(N4CCOCC4)ccnc2N3C(=O)OC(C)(C)C)CC1]

A solution of Intermediate 93 (226 mg, 0.359 mmol) and Cs₂CO₃ (234 mg, 0.718 mmol, 2 eq.) in 1,4-dioxane was degassed with nitrogen. Xantphos (CAS [161265-03-8], 42 mg, 0.072 mmol, 0.2 eq.) and Pd₂(dba)₃ (33 mg, 0.036 mmol, 0.1 eq.) were then added. The reaction mixture was degassed again and was stirred at 100° C. for 16 h. The reaction mixture was partitioned between EtOAc and brine. The combined organic layers were dried (MgSO₄), filtered, and concentrated. The residue was purified by flash column chromatography (silica, heptane/EtOAc from 100/0 to 0/100) to give Intermediate 94 (124 mg, yield: 58%) as a yellow solid.

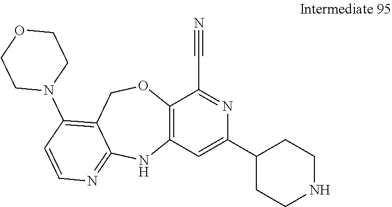 [SMILES: N#Cc1nc(C2CCNCC2)cc2c1OCc1c(N3CCOCC3)ccnc1N2]

TFA (836 μL) was added to a solution of Intermediate 94 (124 mg, 0.209 mmol) in DCM (1 mL), and the mixture was stirred at room temperature for 3 h. The volatiles were evaporated and the residue was taken up with DCM and poured into water/Na₂CO₃ and extracted with DCM. The organic layer was concentrated to afford Intermediate 95 (82 mg, quantitative), used without further purification.

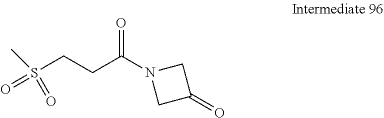

A mixture of 3-(methylsulfonyl)propanoic acid (CAS [645-83-0], 35 g, 230 mmol), EDCI (88.2 g, 460 mmol, 2 eq.), HOBt (45 g. 333.5 mmol, 1.45 eq.), and Et₃N (70.5 mL, 506 mmol, 2.2 eq.) in DCM (1 L) was stirred at room temperature for 30 min. 3-Azetidinone hydrochloride (CAS [17557-84-5], 24.7 g, 230 mmol, 1 eq.) was added and the mixture was stirred at room temperature for 12 h. EtOAc (600 mL) was added to the reaction mixture, and it was stirred for 30 min, filtered, and the filter was rinsed with EtOAc (100 mL×3). The filtrate was evaporated and the residue was purified by column chromatography over silica gel (eluent: EtOAc/MeOH 100/0 to 95/5). The obtained solid was triturated in THF (70 mL), filtered, and dried to afford Intermediate 96 (12.9 g, yield: 27%) as a white solid.

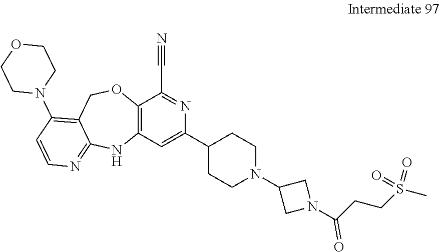 [SMILES: CS(=O)(=O)CCC(=O)N1CC(N2CCC(c3cc4c(c(C#N)n3)OCc3c(N5CCOCC5)ccnc3N4)CC2)C1]

AcOH (23 μL, 0.399 mmol, 1.8 eq.) and molecular sieves (510 mg) were added to a solution of Intermediate 95 (87 mg, 0.222 mmol) and Intermediate 96 (68 mg, 0.333 mmol, 1.5 eq.) in DCM (13 mL), and the mixture was stirred for 1 h. NaBH(OAc)₃ (28 mg, 0.443 mmol, 2 eq.) was added and the mixture was stirred at room temperature for 16 h. More Intermediate 96 (68 mg, 0.333 mmol, 1.5 eq.) was added and the mixture was stirred for 2 h. More NaBH(OAc)₃ (28 mg, 0.443 mmol, 2 eq.) was added and the mixture was stirred at room temperature for 48 h. The mixture was diluted with DCM, and the mixture was filtered. The filtrate was washed with water/NaHCO₃. The organic layer was dried and concentrated to afford Intermediate 97 (119 mg, yield: 92%), used without further purification.

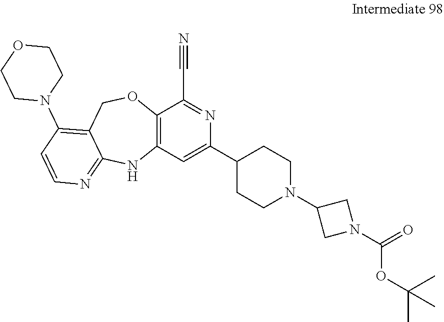 [SMILES: CC(C)(C)OC(=O)N1CC(N2CCC(c3cc4c(c(C#N)n3)OCc3c(N5CCOCC5)ccnc3N4)CC2)C1]

AcOH (44 μL, 2 eq.) was added to a solution of Intermediate 95 (331 mg, 0.39 mmol) and Et₃N (217 μL, 1.56 mmol, 4 eq.) in DCE (10 mL). tert-butyl-3-oxoazetidine-1-carboxylate (CAS [398489-26-4], 100 mg, 0.585 mmol, 1.5 eq.) was added and the reaction mixture was stirred for 1 h. NaBH(OAc)₃ (124 mg, 0.585 mmol, 1.5 eq.) was then added and the mixture was stirred at room temperature for 18 h. More tert-butyl-3-oxoazetidine-1-carboxylate (100 mg, 0.585 mmol, 1.5 eq.) was added again and the mixture was stirred for 1 h. NaBH(OAc)₃ (124 mg, 0.585 mmol, 1.5 eq.) was then added and the mixture was stirred at room temperature overnight. Aqueous NaHCO₃ was added to the reaction mixture and it was extracted with DCM. The organic layer was washed with brine, dried over MgSO₄, filtered, and evaporated. The residue was purified by flash column chromatography (gradient heptane/EtOAc from 5% to 100% followed by MeOH/DCM 0% to 100%) to afford Intermediate 98 (165 mg, yield: 77%).

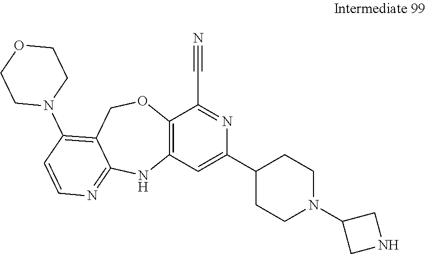 [SMILES: N#Cc1nc(C2CCN(C3CNC3)CC2)cc2c1OCc1c(N3CCOCC3)ccnc1N2]

Intermediate 98 (165 mg, 0.301 mmol) was dissolved in DCM (2 mL) at room temperature and TFA (1.2 mL) was added. The reaction mixture was stirred at room temperature for 3 h. The volatiles were evaporated to afford Intermediate 99 (134 mg, quantitative), used without further purification.

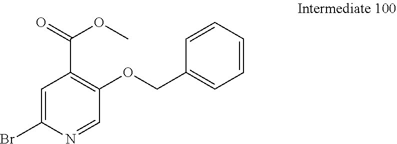 [SMILES: COC(=O)c1cc(Br)ncc1OCc1ccccc1]

Thionyl chloride (7.48 mL, 103.193 mmol, 1.3 eq.) was added dropwise to a solution of 2-bromo-5-(phenylmethoxy)-4-pyridinecarboxylic acid (CAS [1256823-39-8], 24.46 g, 79.38 mmol) in MeOH (180 mL). The reaction mixture was refluxed for 1 h. The reaction mixture was poured into aqueous NaHCO₃ and the pH was adjusted to 7. The mixture was extracted with DCM and the organic layer was dried over MgSO₄. The solvent was evaporated to give Intermediate 100 (20.36 g, yield: 78%), used without further purification.

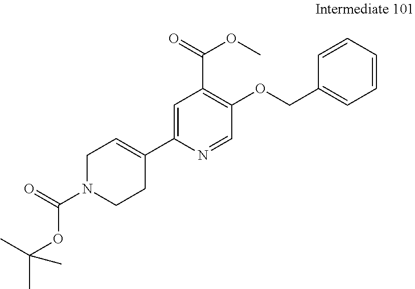 [SMILES: COC(=O)c1cc(C2=CCN(C(=O)OC(C)(C)C)CC2)ncc1OCc1ccccc1]

Intermediate 100 (10.455 g, 32.454 mmol) and N-Boc-1,2,3,6-tetrahydropyridine-4-boronic acid pinacol ester (CAS [286961-14-6], 11.039 g, 35.7 mmol, 1.1 eq.) were dissolved in 1,4-dioxane. Na₂CO₃ (1 M in water, 48.7 mL, 48.681 mmol, 1.5 eq.) was added and the mixture was degassed with nitrogen for 15 min. PdCl₂(PPh₃)₂(CAS [13965-03-2], 1.367 g, 1.947 mmol, 0.06 eq.) was then added, and the reaction mixture was stirred at 80° C. under nitrogen atmosphere for 6 h. After cooling, the mixture was diluted with EtOAc (100 mL) and water (50 mL). The mixture was filtered through a pad of Celite that was further rinsed with EtOAc (2×50 mL). The organic layer of the filtrate was separated, dried over MgSO₄, filtered, and concentrated. The residue was purified by column chromatography over silica gel (DCM/MeOH/NH₃ 9/0.9/0.1 in DCM from 0 to 100%) to give Intermediate 101 (12.404 g, yield: 84%).

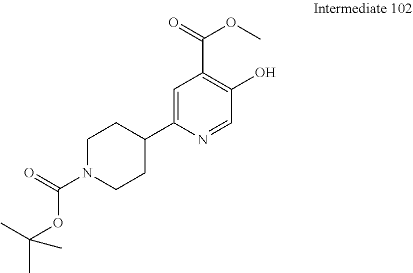 [SMILES: COC(=O)c1cc(C2CCN(C(=O)OC(C)(C)C)CC2)ncc1O]

Intermediate 101 (12.404 g, 20.455 mmol) was dissolved in MeOH and the solution was cooled to 0° C. under nitrogen atmosphere. Pd/C 10% (1.322 g) was added and the reaction vessel was connected to a balloon filled with hydrogen. The mixture was stirred under atmosphere of hydrogen for 5 days at room temperature. The catalyst was filtered off and the filtrate was concentrated to give Intermediate 102 (6.88 g, yield: 80%) as an oil, used without further purification.

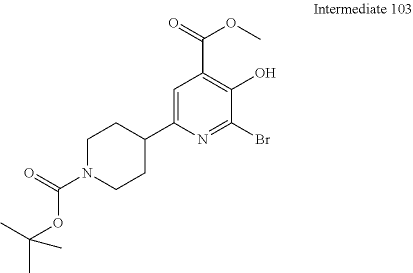 [SMILES: COC(=O)c1cc(C2CCN(C(=O)OC(C)(C)C)CC2)nc(Br)c1O]

NBS (6.376 g, 35.824 mmol, 1.1 eq.) was added to a solution of Intermediate 102 (10.955 g, 32.567 mmol) in DMF (152 mL) at 0° C. The resulting mixture was stirred for 1.5 h. The reaction mixture was poured into water and the mixture was extracted with DCM/MeOH (9/1). The organic layer was dried over MgSO₄, filtered, and concentrated. The residue was purified by column chromatography over silica gel (gradient EtOAc in heptane from 0% to 80%) to afford Intermediate 103 (6.263 g, yield: 46%) as an off-white solid.

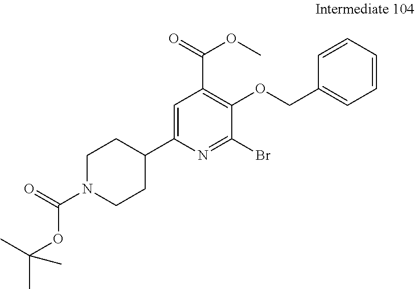 [SMILES: COC(=O)c1cc(C2CCN(C(=O)OC(C)(C)C)CC2)nc(Br)c1OCc1ccccc1]

Benzyl bromide (2.51 mL, 21.114 mmol, 1.4 eq.) was added to a solution of Intermediate 103 (6.263 g, 15.081 mmol) and K₂CO₃ (4.17 g, 20.163 mmol, 2 eq.) in DMF (80 mL). The reaction mixture was stirred at room temperature overnight. The mixture was filtered. Water and brine were added and the mixture was extracted with EtOAc. The organic layer was dried over MgSO₄, filtered, and evaporated. The residue was purified by column chromatography over silica gel (gradient of EtOAc in heptane from 0% to 20%) to afford Intermediate 104 (6.485 g, yield: 79%) as a solid.

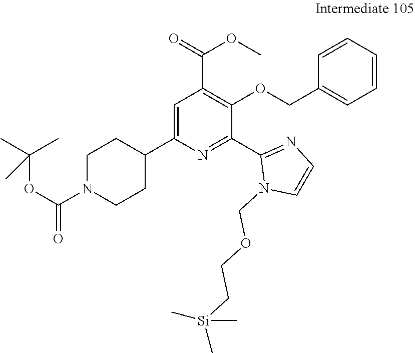 [SMILES: COC(=O)c1cc(C2CCN(C(=O)OC(C)(C)C)CC2)nc(-c2nccn2COCC[Si](C)(C)C)c1OCc1ccccc1]

Tetrakis(triphenylphosphine)-palladium (CAS [14221-01-3], 1.33 g, 1.151 mmol, 0.1 eq.) was added to a mixture of Intermediate 104 (5.816 g, 11.508 mmol) and 2-(tributylstannyl)-1-((2-(trimethylsilyl)ethoxy)methyl)-1H-imidazole (CAS [1449143-14-9], 5.609 g, 11.508 mmol, 1 eq.) in toluene (60 mL) under nitrogen atmosphere in a sealed tube. The reaction mixture was stirred at 100° C. for 5 h. The solvent was evaporated and the residue was purified by column chromatography over silica gel (gradient of EtOAc in heptane from 0% to 70%) to afford Intermediate 105 (5.78 g, yield: 79%) as an oil.

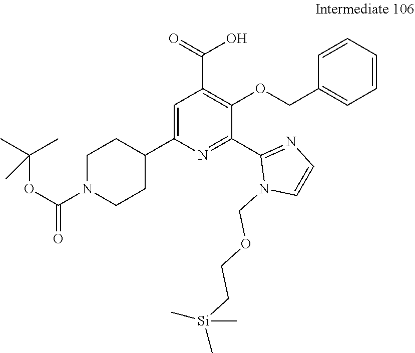 [SMILES: CC(C)(C)OC(=O)N1CCC(c2cc(C(=O)O)c(OCc3ccccc3)c(-c3nccn3COCC[Si](C)(C)C)n2)CC1]

LiOH (779 mg, 18.561 mmol, 2 eq.) was added to a solution of Intermediate 105 (5.78 g, 9.28 mmol) in THE (36 mL) and water (9 mL) at room temperature. The reaction mixture was stirred at room temperature for 4 h. The pH was brought to 7 by addition of KHSO₄ (1 M in water) and the mixture was concentrated to dryness to give Intermediate 106 (5.149 g, yield: 90%) as a solid, used without further purification.

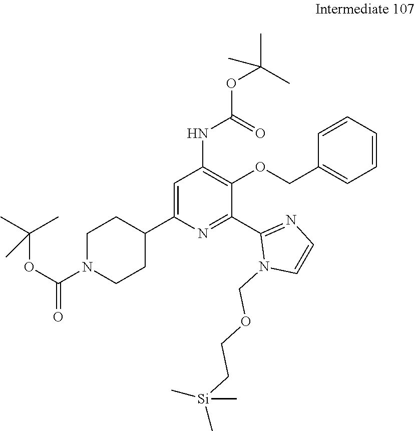 [SMILES: CC(C)(C)OC(=O)Nc1cc(C2CCN(C(=O)OC(C)(C)C)CC2)nc(-c2nccn2COCC[Si](C)(C)C)c1OCc1ccccc1]

DPPA (CAS [26386-88-9], 3.645 mL, 16.915 mmol, 2 eq.) was added to a solution of Intermediate 106 (5.149 g, 8.458 mmol) and Et₃N (1.53 mL, 10.995 mmol, 1.3 eq.) in tBuOH (56 mL) at room temperature under nitrogen atmosphere. The mixture was stirred at 70° C. for 7 h. After cooling, the mixture was diluted with DCM and Na₂CO₃ (1 M in water). The layers were separated and the organic layer was dried over MgSO₄, filtered, and evaporated. The residue was purified by column chromatography over silica gel (gradient DCM:MeOH (9:1) in DCM from 0% to 50%) to afford Intermediate 107 (3.894 g, yield: 66%) as an oil.

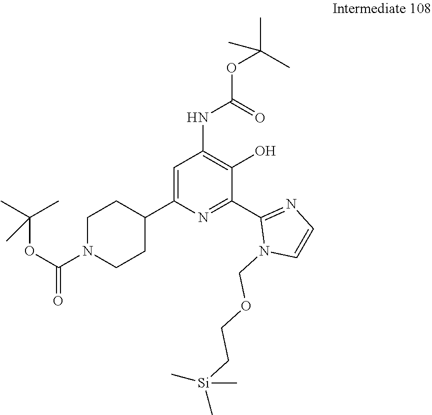 [SMILES: CC(C)(C)OC(=O)Nc1cc(C2CCN(C(=O)OC(C)(C)C)CC2)nc(-c2nccn2COCC[Si](C)(C)C)c1O]

Pd/C 10% (390 mg) was added to a cold solution of Intermediate 107 (3.894 g, 5.727 mmol) in MeOH under nitrogen atmosphere. Then the reaction vessel was evacuated and filled with hydrogen (5 times). The reaction mixture was stirred at room temperature for 5 h. The mixture was filtered through a pad of celite and the filter cake was washed with MeOH (5×50 mL). The filtrate was evaporated to give Intermediate 108 (2.965 g, yield: 83%) as an oil.

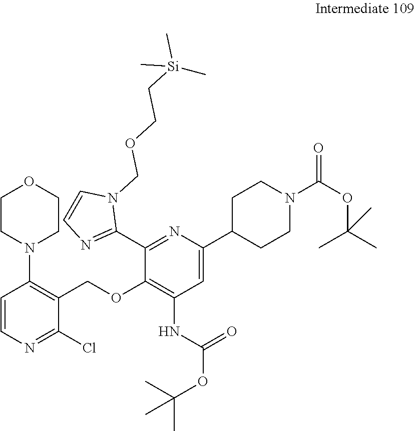 [SMILES: CC(C)(C)OC(=O)Nc1cc(C2CCN(C(=O)OC(C)(C)C)CC2)nc(-c2nccn2COCC[Si](C)(C)C)c1OCc1c(N2CCOCC2)ccnc1Cl]

Intermediate 60 (1.682 g, 6.806 mmol, 1.2 eq.) was added to a mixture of Intermediate 108 (3.345 g, 5.671 mmol) and K₂CO₃ (1.02 g, 7.373 mmol, 1.3 eq.) in DMF (40 mL). The reaction mixture was stirred at room temperature overnight. Water and EtOAc were added and the layers were separated. The organic layer was dried over MgSO₄, filtered, and evaporated. The residue was purified by column chromatography over silica gel (gradient of DCM/MeOH (9/1) in DCM from 0% to 45%) to afford Intermediate 109 (4.326 g, yield: 86%) as an oil.

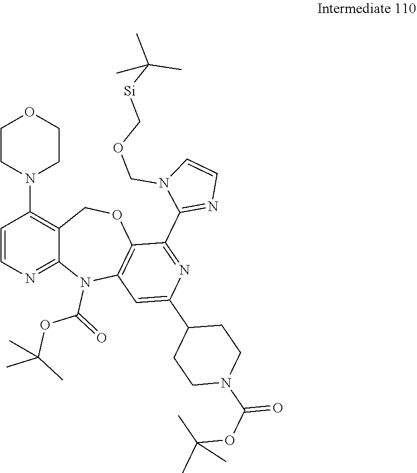

Intermediate 109 (4.326 g, 5.404 mmol) and Cs₂CO₃ (2.641 g, 8.107 mmol, 1.5 eq.) were suspended in toluene (60 mL) and this mixture was degassed with nitrogen for 15 min. Pd(OAc)₂ (63 mg, 0.54 mmol, 0.1 eq.) and Xantphos (CAS [161265-03-8], 313 mg, 0.54 mmol, 0.1 eq.) were then added and the resulting mixture was stirred at 120° C. under nitrogen atmosphere overnight. After cooling, the reaction mixture was diluted with water (100 mL) and EtOAc (150 mL). The layers were separated and the organic laver was dried over MgSO₄, filtered, and concetrated. The residue was purified by column chromatography over silica gel (gradient of DCM:MeOH (9:1) in DCM from 0% to 50%) to afford Intermediate 110 (2.756 g, yield: 58%) as an oil.

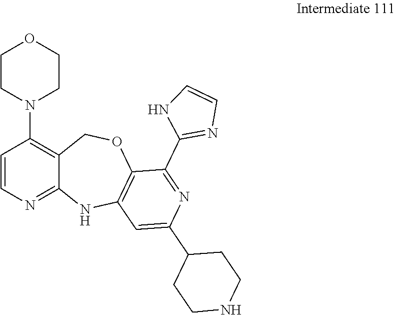 [SMILES: c1c[nH]c(-c2nc(C3CCNCC3)cc3c2OCc2c(N4CCOCC4)ccnc2N3)n1]

TFA (414 μL, 5.4 mmol, 10 eq.) was added to a mixture of Intermediate 110 (646 mg, 0.54 mmol) in DCM (10 mL). The mixture was stirred at room temperature overnight. To push the reaction to completion, more TFA (414 μL, 5.4 mmol, 10 eq.) was added. The reaction mixture was stirred at room temperature overnight. The volatiles were evaporated and the residue was washed with toluene twice and dried to give Intermediate 111 (522 mg, quantitative), used without further purification.

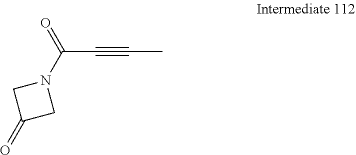

2-Butynoic acid (55.9 g, 664.9 mmol, 1.1 eq.) and Et₃N (253 mL, 1813 mmol, 3 eq.) were dissolved in DCM (1 L) and stirred at 0° C. Azetidin-3-one (CAS [17557-84-5], 65 g, 604 mmol) was added to the reaction mixture in one portion. Propylphosphonic anhydride (CAS [68957-94-8], 577 g, 907 mmol, 1.5 eq.) was then added slowly and the mixture was stirred at 0° C. for 4 h. Water (800 mL) was added slowly to the mixture and the cooling bath was removed. The mixture was extracted with DCM:MeOH 10:1 (4×1 L). The combined organic layers were dried (MgSO₄) and concentrated. The residue was purified by column chromatography over silica gel (eluent: petroleum ether/EtOAc 100/0 to 50/50). The obtained solid was triturated in MTBE (100 mL), filtered, and dried to afford Intermediate 112 (53.1 g, yield: 48%) as a white solid.

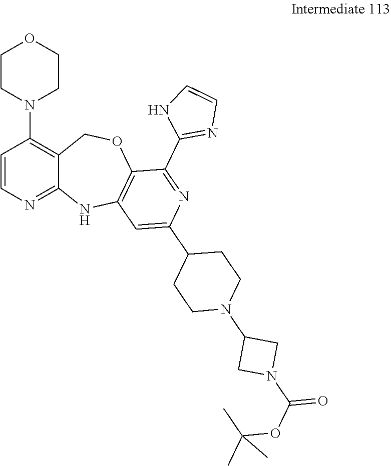 [SMILES: CC(C)(C)OC(=O)N1CC(N2CCC(c3cc4c(c(-c5ncc[nH]5)n3)OCc3c(N5CCOCC5)ccnc3N4)CC2)C1]

N-Boc-3-oxoazetidine (CAS [398489-26-4]. 318 mg, 1; 855 mmol, 2 eq.) and AcOH (53 μL, 0.927 mmol, 1 eq.) were added to a solution of Intermediate 111 (402 mg, 0.927 mmol) in DCE (15 mL). The reaction mixture was stirred for 30 min. NaBH(OAc)₃ (295 mg, 1.391 mmol, 1.5 eq.) was added portionwise and the mixture was stirred at room temperature for 5 h. DCM and Na₂CO₃ (1 M in water) were added and the layers were separated. The organic layer was dried over MgSO₄, filtered, concentrated, and purified by column chromatography over silica gel (gradient of DCM/MeOH/NH₃ (9/0.9/1) in DCM from 0% to 100%) to give Intermediate 113 (323 mg, yield: 58%) as an oil.

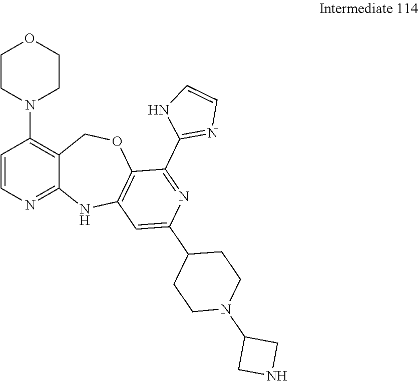 [SMILES: c1c[nH]c(-c2nc(C3CCN(C4CNC4)CC3)cc3c2OCc2c(N4CCOCC4)ccnc2N3)n1]

TFA (840 μL, 10.973 mmol, 20 eq.) was added to solution of Intermediate 113 (323 mg, 0.549 mmol) in DCM (10 mL). The reaction mixture was stirred at room temperature overnight. The volatile were evaporated. Na₂CO₃ (1 M in water) was added to the residue and the mixture was extracted with DCM/MeOH (7/1). The organic layer was dried over MgSO₄, filtered, and evaporated to give Intermediate 114 (220 mg yield: 81%), used without further purification.

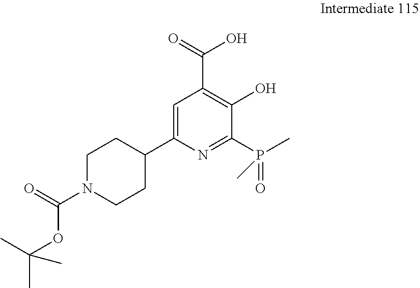 [SMILES: CC(C)(C)OC(=O)N1CCC(c2cc(C(=O)O)c(O)c(P(C)(C)=O)n2)CC1]

Intermediate 103 (5 g, 12.04 mmol), dimethylphosphine oxide (CAS [7211-39-4], 1.879 g, 24.08 mmol, 2 eq.), and K₃PO₄ (2.811 g, 13.244 mmol, 1.1 eq.) were stirred in dry DMF (60 mL) under nitrogen atmosphere for 15 min. Pd(OAc)₂ (270 mg, 1.204 mmol, 0.1 eq.) and Xantphos (CAS [161265-03-8], 1.204 mmol, 0.1 eq.) were added and the mixture was stirred at 90° C. overnight. Na₂CO₃ (1 M in water) was added and the mixture was extracted with DCM. The organic layer was dried over MgSO₄, filtered, and concentrated. The residue was purified by column chromatography over silica gel (gradient of MeOH in DCM from 0% to 10%) to give the impure methyl ester of Intermediate 115 as a brown oil (2.831 mg). The aqueous layer was brought to pH 5-6 and was extracted with DCM and DCM/MeOH. The organic layer was dried over MgSO₄, filtered, and concentrated to give Intermediate 115 (2.392 g, yield: 46%) as a yellow oil.

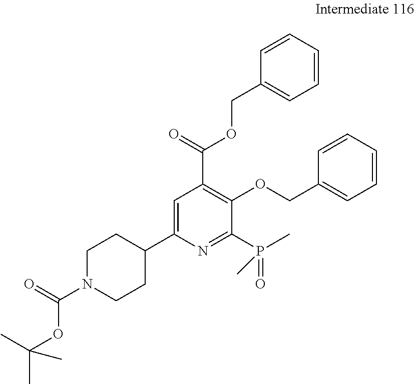 [SMILES: CC(C)(C)OC(=O)N1CCC(c2cc(C(=O)OCc3ccccc3)c(OCc3ccccc3)c(P(C)(C)=O)n2)CC1]

Benzyl bromide (1.785 mL, 15.01 mmol, 2.5 eq.) was added to a solution of Intermediate 115 (2.392 g, 6.004 mmol) and K₂CO₃ (0.996 g, 7.205 mmol, 1.2 eq.) in DMF (55 mL). The raction mixture was stirred at room temperature overnight. The reaction mixture was filtered and Na₂CO₃ (1 M in water) was added. The mixture was extracted in DCM. The organic layer was dried over MgSO₄, filtered, concentrated, and purified by column chromatography over silica gel (gradient of DCM/MeOH (9/1) in DCM from 0% to 80%) to give Intermediate 116 (3.317 g. yield: 76%) as an oil.

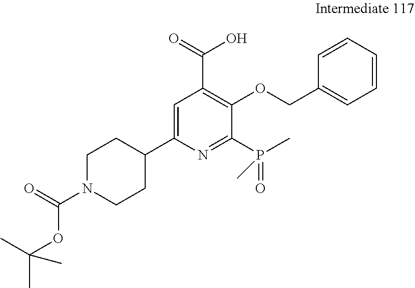 [SMILES: CC(C)(C)OC(=O)N1CCC(c2cc(C(=O)O)c(OCc3ccccc3)c(P(C)(C)=O)n2)CC1]

NaOH (1 M in water, 28.5 mL, 28.515 mmol, 5 eq.) was added to a solution of Intermediate 116 (3.3 g, 5.703 mmol) in MeOH (15 mL). The reaction mixture was stirred at room temperature overnight. The pH was brought to 6-7 with KHSO₄ (1 M in water). The mixture was extracted with DCM and DCM/MeOH (4/1). The organic layer was evaporated. The residue was triturated in Et₂O, filtered, and dried to afford Intermediate 117 (1.2 g, yield: 43%), used without further purification.

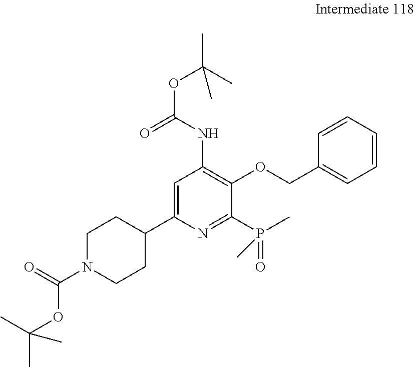 [SMILES: CC(C)(C)OC(=O)Nc1cc(C2CCN(C(=O)OC(C)(C)C)CC2)nc(P(C)(C)=O)c1OCc1ccccc1]

DPPA (CAS[26386-88-9], 2.25 mL, 10.44 mmol, 3 eq.) was added to a solution of Intermediate 117 (1.7 g, 3.48 mmol) and Et₃N (631 μL, 4.254 mol, 1.3 eq.) in tBuOH (26 mL) at room temperature under nitrogen atmosphere. The mixture was refluxed for 3 h. After cooling, Na₂CO₃ (1 M in water) was added and the mixture was extracted with DCM. The organic layer was dried over MgSO₄, filtered, and concentrated. The residue was purified by column chromatography over silica gel (gradient of MeOH in DCM/MeOH (9/1) from 0% to 60%) to give Intermediate 118 (356 mg, yield: 16%).

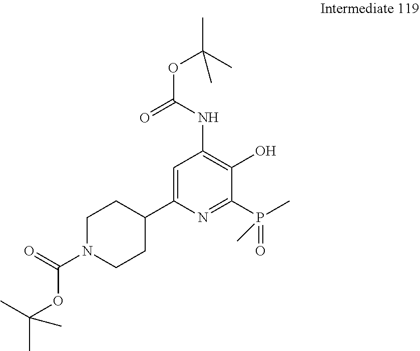 [SMILES: CC(C)(C)OC(=O)Nc1cc(C2CCN(C(=O)OC(C)(C)C)CC2)nc(P(C)(C)=O)c1O]

Intermediate 118 (356 mg, 0.604 mmol) was dissolved in MeOH (100 mL) and cooled to 0° C. under nitrogen atmosphere. Pd/C 10% (39 mg) was added and the mixture was stirred under an atmosphere of hydrogen (atmospheric pressure) for 48 h at room temperature. The catalyst was filtered off and the filtrate was concentrated to give Intermediate 119 (284 mg, yield: 97%) as a brown oil, used without further purification.

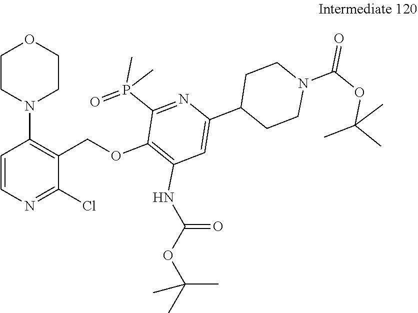 [SMILES: CC(C)(C)OC(=O)Nc1cc(C2CCN(C(=O)OC(C)(C)C)CC2)nc(P(C)(C)=O)c1OCc1c(N2CCOCC2)ccnc1Cl]

Intermediate 119 (286 mg, 0.609 mmol) was added to a mixture of Intermediate 60 (166 mg, 0.67 mmol, 1.1 eq.) and K₂CO₃ (168 mg, 1.218 mmol, 2 eq.) in DMF (50 mL). The reaction mixture was stirred at room temperature overnight. Water and DCM were added and the layers were separated. The organic layer was dried over MgSO₄, filtered, concentrated, and purified by column chromatography over silica gel (gradient of DCM/MeOH (9/1) in DCM from 0% to 100%) to afford Intermediate 120 (291 mg, yield: 64%) as a brown oil.

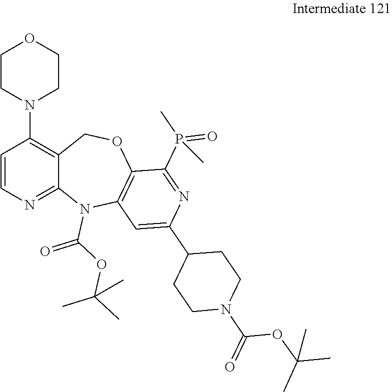 [SMILES: CC(C)(C)OC(=O)N1CCC(c2cc3c(c(P(C)(C)=O)n2)OCc2c(N4CCOCC4)ccnc2N3C(=O)OC(C)(C)C)CC1]

Intermediate 120 (291 mg, 0.428 mmol) and Cs₂CO₃ (209 mg, 0.642 mmol, 1.5 eq.) were suspended in toluene (15 mL) and the mixture was degassed with nitrogen for 15 min. Palladium (II) acetate (10 mg, 0.043 mmol, 0.1 eq.) and Xantphos (CAS [161265-03-8], 25 mg, 0.043 mmol, 0.1 eq.) were then added and the resulting mixture was refluxed under nitrogen atmosphere overnight. As the reaction did not proceed, after cooling. Pd₂(dba)₃ (404 mg, 0.428 mmol, 1 eq.) and Xantphos (CAS [161265-03-8], 25 mg, 0.043 mmol, 0.1 eq.) were added to the mixture and the reaction was refluxed under nitrogen atmosphere overnight. The reaction mixture was diluted with water (40 mL) and was extracted with EtOAc (2×50 mL). The combined organic layers were washed with brine, dried over MgSO₄, filtered, concentrated, and purified by chromatography over silica gel (DCM/MeOH 9/1 in DCM from 0 to 40%) to afford Intermediate 121 (157 mg, yield: 57%) as a brown oil.

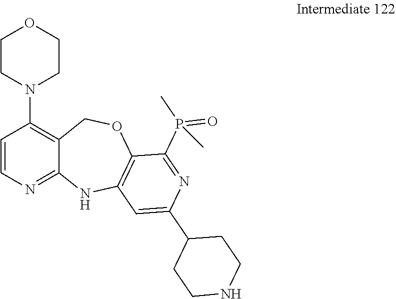 [SMILES: CP(C)(=O)c1nc(C2CCNCC2)cc2c1OCc1c(N3CCOCC3)ccnc1N2]

TFA (187 μL, 2.439 mmol, 10 eq.) was added to solution of Intermediate 121 (157 mg, 0.244 mmol) in DCM (10 mL). The reaction mixture was stirred overnight at room temperature. The volatiles were evaporated to give Intermediate 122 (219 mg, yield: 90%), used without further purification.

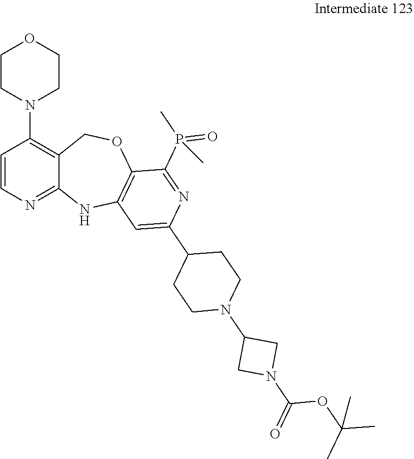 [SMILES: CC(C)(C)OC(=O)N1CC(N2CCC(c3cc4c(c(P(C)(C)=O)n3)OCc3c(N5CCOCC5)ccnc3N4)CC2)C1]

NaBH(OAc)₃ (155 mg, 0.73 mmol, 3 eq.) was added to a solution of Intermediate 122 (219 mg, 0.243 mmol), Et₃N (135 μL, 0.974 mmol, 4 eq.), and N-Boc-3-oxoazetidine (CAS [398489-26-4], 125 mg, 0.73 mmol, 3 eq.) in DCE (20 mL). The reaction mixture was stirred at room temperature overnight. Na₂CO₃ (1 M in water) was added and the mixture was extracted with DCM. The organic layer was dried over MgSO₄, filtered, and concentrated. The residue was purified by column chromatography over silica gel (gradient of DCM/MeOH (9/1) in DCM from 0% to 50%) to afford Intermediate 123 (101 mg, yield: 69%) as a solid.

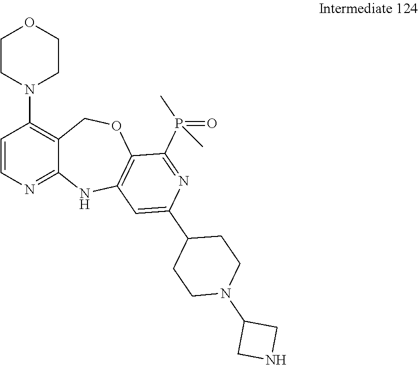 [SMILES: CP(C)(=O)c1nc(C2CCN(C3CNC3)CC2)cc2c1OCc1c(N3CCOCC3)ccnc1N2]

TFA (258 μL, 3.374 mmol, 20 eq.) was added to solution of Intermediate 123 (101 mg, 0.169 mmol) in DCM (10 mL). The reaction mixture was stirred overnight at room temperature. The volatiles were evaporated to give Intermediate 124 (84 mg, yield: 90%), used without further purification.

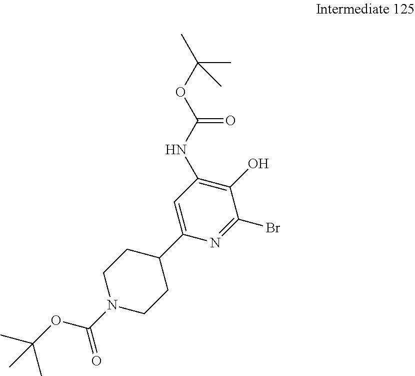 [SMILES: CC(C)(C)OC(=O)Nc1cc(C2CCN(C(=O)OC(C)(C)C)CC2)nc(Br)c1O]

NBS (1.493 g, 8.387 mmol, 1.1 eq.) was added to a solution of Intermediate 30 (3 g, 7.264 mmol) in DMF (75 mL) at 0° C. The resulting mixture was stirred for 2 h. More NBS (271 mg, 1.524 mmol, 0.2 eq.) was added and the reaction mixture was stirred for 2 h. The reaction mixture was diluted with EtOAc and was washed with brine (×5), dried over MgSO₄, filtered, and concentrated to give Intermediate 125 (3.557 g, quantitative), used without further purification.

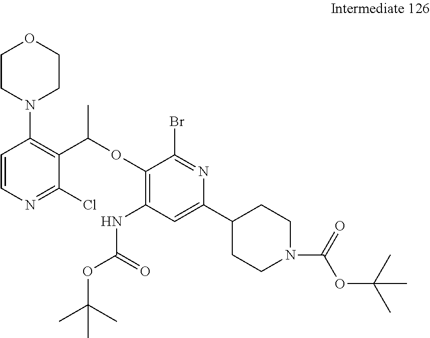 [SMILES: CC(Oc1c(NC(=O)OC(C)(C)C)cc(C2CCN(C(=O)OC(C)(C)C)CC2)nc1Br)c1c(N2CCOCC2)ccnc1Cl]

K₂CO₃ (4.163 g, 30.12 mmol, 4 eq.) was added to a suspension of Intermediate 125 (3.557 g, 7.53 mmol) in DMF (25 mL). The reaction mixture was stirred at room temperature and Intermediate 27 (3.441 g, 13.178 mmol, 1.75 eq.) was added in small portions over 5 h. The mixture was stirred at room temperature for 16 h. The reaction mixture was diluted with EtOAc and washed with brine (5 x). The organic layer was concentrated and the residue was purified by column flash chromatography (SiO₂, heptane:DCM (9:1)/EtOAc) to give Intermediate 126 (4.21 g, yield: 80%).

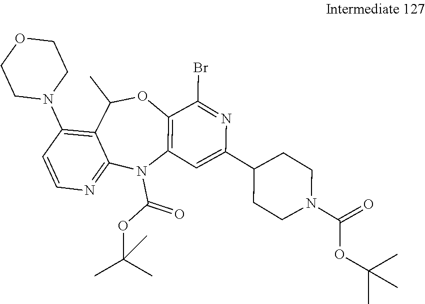 [SMILES: CC1Oc2c(cc(C3CCN(C(=O)OC(C)(C)C)CC3)nc2Br)N(C(=O)OC(C)(C)C)c2nccc(N3CCOCC3)c21]

A solution of Intermediate 126 (3.68 g, 5.279 mmol) and Cs₂CO₃ (2.58 g, 7.919 mmol, 1.5 eq.) in toluene (220 mL) was degassed with nitrogen. Then, S-Phos (CAS [657408-07-6], 325 mg, 0.792 mmol, 0.15 eq.) and Pd(II) acetate (178 mg, 0.792 mmol, 0.15 eq.) were added. The reaction mixture was degassed again with nitrogen and it was stirred at 100° C. for 15 h. More Cs₂CO₃ (2.58 g, 7.919 mmol, 1.5 eq.), S-Phos (CAS [657408-07-6], 325 mg, 0.792 mmol, 0.15 eq.) and Pd(II) acetate (178 mg, 0.792 mmol, 0.15 eq.) were added and the reaction was further stirred at 100° C. overnight. The residue was purified by column flash chromatography (SiO₂, EtOAc-heptane gradient) to give Intermediate 127 (1.221 g, yield: 35%).

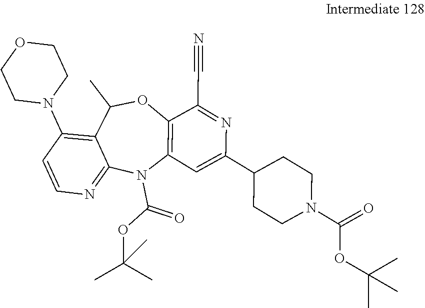 [SMILES: CC1Oc2c(cc(C3CCN(C(=O)OC(C)(C)C)CC3)nc2C#N)N(C(=O)OC(C)(C)C)c2nccc(N3CCOCC3)c21]

A mixture of Intermediate 127 (1.052 g, 1.592 mmol) and zinc dust (125 mg, 1.911 mmol, 1.2 eq.) in DMA (55 mL) under nitrogen was stirred for 10 min. Then, Zn(CN)₂ (748 mg, 6.37 mmol, 4 eq.) and Pd(dppf)Cl₂·DCM, (CAS [95464-05-4], 261 mg, 0.318 mmol, 0.2 eq.) was added and the mixture was stirred at 100° C. for 16 h. Water and EtOAc were added and the layers were separated. The organic layer was dried over MgSO₄, filtered, and evaporated. The residue was purified by flash column chromatography on silica gel (heptane-EtOAc then DCM-MeOH) to afford Intermediate 128 (1.058 g, quantitative)

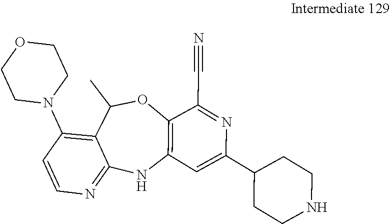 [SMILES: CC1Oc2c(cc(C3CCNCC3)nc2C#N)Nc2nccc(N3CCOCC3)c21]

Intermediate 128 (1.058 g, 1.744 mmol) was dissolved in a mixture of TFA (4 mL) and DCM (6 mL) and the reaction mixture was stirred for 3 h at room temperature. The volatiles were evaporated and the residue was co-evaporated with toluene (2×100 mL) to give Intermediate 129 (2.697 g, quantitative), used without further purification.

### Intermediate 130, Intermediate 131, and Intermediate 132

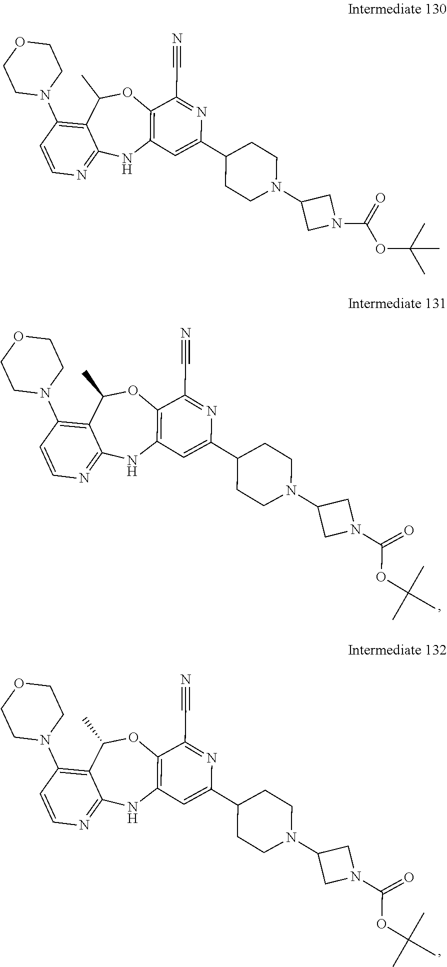 [SMILES: CC1Oc2c(cc(C3CCN(C4CN(C(=O)OC(C)(C)C)C4)CC3)nc2C#N)Nc2nccc(N3CCOCC3)c21 ; C[C@H]1Oc2c(cc(C3CCN(C4CN(C(=O)OC(C)(C)C)C4)CC3)nc2C#N)Nc2nccc(N3CCOCC3)c21 ; C[C@@H]1Oc2c(cc(C3CCN(C4CN(C(=O)OC(C)(C)C)C4)CC3)nc2C#N)Nc2nccc(N3CCOCC3)c21]

Both Intermediate 131 and Intermediate 132 are pure enantiomers but their absolute stereochemistry is undetermined tert-Butyl-3-oxoazetidine-1-carboxylate (CAS [398489-26-4], 597 mg, 3.488 mmol, 2 eq.) was added to a solution of Intermediate 129 (1.902 g, 1.744 mmol) and Et₃N (1.45 mL, 10.464 mmol, 6 eq.) in DCE (75 mL). The reaction mixture was stirred for 1 h at room temperature. Then, NaBH(OAc)₃ (739 mg, 3.488 mmol, 2 eq.) was added and the mixture was stirred at room temperature for 18 h. NaHCO₃ (1 M in water) was added to the reaction mixture and it was extracted with DCM. The organic layer was washed with brine, dried over MgSO₄, filtered, and evaporated. The residue was purified by flash column chromatography on silica gel (gradient heptane/EtOAc from 5% to 100%) to give Intermediate 130 (788 mg, yield: 80%). Intermediate 130 was separated into its enantiomers by chiral SFC (Stationary phase: Chiralpak IG 5 μm 250*20 mm, Mobile phase: 50% CO₂, 50% mixture of EtOH/DCM 80/20 v/v (+0.3% iPrNH₂)) to give Intermediate 131 (258 mg, yield: 33%) and Intermediate 132 (254 mg, yield: 32%).

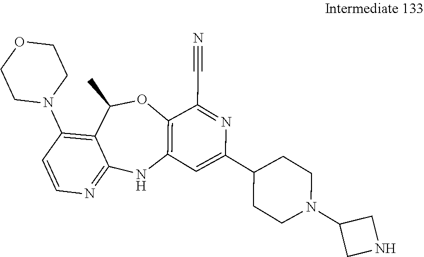 [SMILES: C[C@H]1Oc2c(cc(C3CCN(C4CNC4)CC3)nc2C#N)Nc2nccc(N3CCOCC3)c21]

(*R), Pure enantiomer but absolute stereochemistry undetermined TFA (1.7 mL, 22.215 mmol, 48 eq.) was added to a solution of Intermediate 131 (258 mg, 0.459 mmol) in DCM (3 mL) at 0° C. The reaction mixture was stirred for 3 h at room temperature. The volatiles were evaporated and the residue was taken up in DCM and a few drops of MeOH. This solution was basified with NH₄OH (30% in water). The layers were separated and the organic layer was evaporated to give Intermediate 133 (220 mg, quantitative), used without further purification.

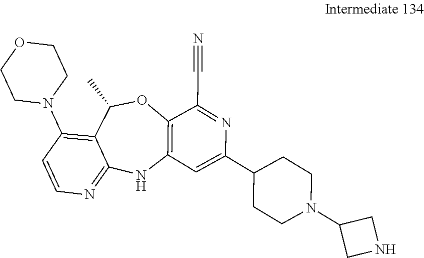 [SMILES: C[C@@H]1Oc2c(cc(C3CCN(C4CNC4)CC3)nc2C#N)Nc2nccc(N3CCOCC3)c21]

(*S), Pure enantiomer but absolute stereochemistry undetermined Intermediate 134 was prepared using a procedure analogous to Intermediate 133, using Intermediate 132 instead of Intermediate 131.

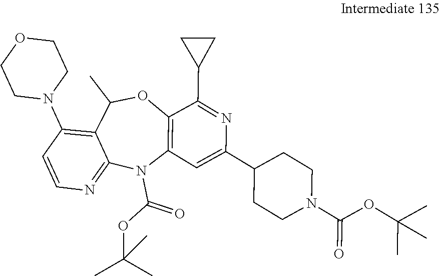 [SMILES: CC1Oc2c(cc(C3CCN(C(=O)OC(C)(C)C)CC3)nc2C2CC2)N(C(=O)OC(C)(C)C)c2nccc(N3CCOCC3)c21]

A solution of Intermediate 127 (407 mg, 0.616 mmol), cyclopropyl boronic acid (159 mg, 1.848 mmol, 3 eq.), and K₃PO₄ (392 mg, 1.848 mmol, 3 eq.) in 1,4-dioxane (3 mL) and water (0.6 mL) was degassed with nitrogen. Then, dichloro[1,1′-bis(diphenylphosphino)ferrocene]palladium(II), complex with DCM (1:1) (CAS [95464-05-4], 50 mg, 0.062 mmol, 0.1 eq.) was added. The reaction mixture was degassed again with nitrogen and stirred at 80° C. for 24 h. The reaction mixture was partitioned between EtOAc and brine. The combined organic layers were concentrated and the residue was purified by column flash chromatography on silica gel (EtOAc/heptane:DCM (9:1)) to give Intermediate 135 (215 mg, yield: 56%).

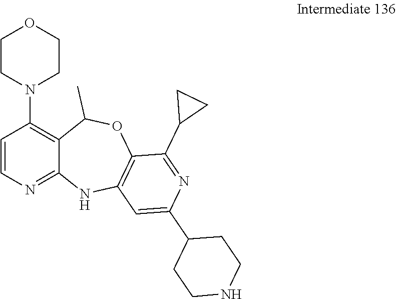 [SMILES: CC1Oc2c(cc(C3CCNCC3)nc2C2CC2)Nc2nccc(N3CCOCC3)c21]

Intermediate 135 (770 mg, 1.238 mmol) was dissolved in DCM (12 mL) at room temperature and TFA (8 mL) was added. The reaction mixture was stirred at room temperature for 3 h. The volatiles were evaporated to give Intermediate 136 (1.18 g, quantitative), used without further purification.

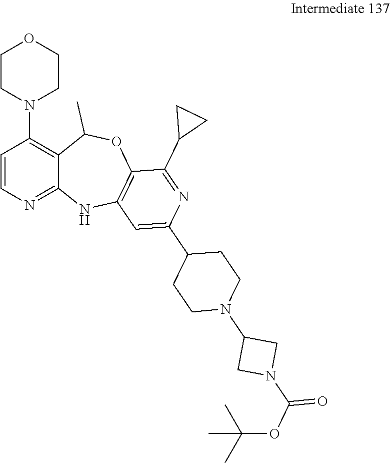 [SMILES: CC1Oc2c(cc(C3CCN(C4CN(C(=O)OC(C)(C)C)C4)CC3)nc2C2CC2)Nc2nccc(N3CCOCC3)c21]

N-Boc-3-oxoazetidine(CAS [398489-26-4], 460 mg, 2.689 mmol, 2 eq.) was added to a solution of Intermediate 136 (1180 mg, 1.345 mmol) and Et₃N (748 μL, 5.378 mmol, 4 eq.) in DCE (10 mL), and the reaction mixture was stirred for 1 h. Then, NaBH(OAc)₃ (427 mg, 2.017 mmol, 1.5 eq.) was added and the mixture was stirred at room temperature for 24 h. Aqueous NaHCO₃ was added to the reaction mixture and it was extracted with DCM. The organic layer was washed with brine, dried over MgSO₄, filtered, and evaporated. The residue was purified by flash column chromatography on silica gel (gradient heptane/EtOAc from 50% to 100%, then MeOH/DCM (0% to 10%)) to afford Intermediate 137 (450 mg, yield: 58%).

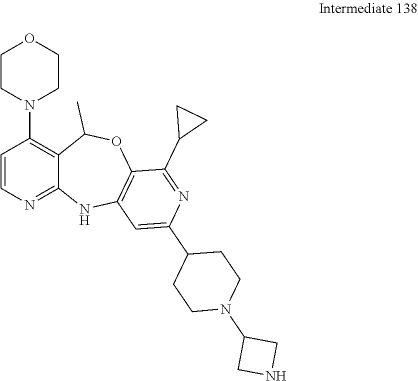 [SMILES: CC1Oc2c(cc(C3CCN(C4CNC4)CC3)nc2C2CC2)Nc2nccc(N3CCOCC3)c21]

Intermediate 137 (450 mg, 0.78 mmol) was dissolved in DCM (12 mL) at room temperture and TFA (8 mL) was added. The reaction mixture was stirred at room temperature for 3 h. The volatiles were evaporated to give Intermediate 138 (372 mg, quantitative), used without further purification.

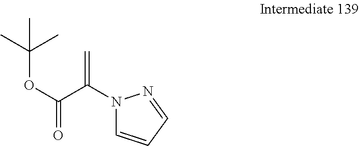

Triphenylphosphine (3.119 g, 11.89 mmol, 1 eq.) was added to a solution of tert-butyl propiolate (1.5 g, 11.89 mmol) and pyrazole (1.619 g, 23.781 mmol, 2 eq.) in DCM (10 mL). The reaction mixture was stirred at room temperature for 16 h. The reaction was quenched by addition of saturated aqueous NaHCO₃. The mixture was extracted with DCM. The organic layer was concentrated under vacuum and the residue was purified by column flash chromatography (SiO₂, DCM/MeOH) to give Intermediate 139 (830 mg, yield: 36%) as a solid.

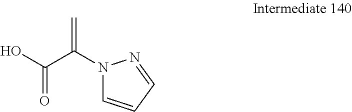

Intermediate 139 (202 mg, 1.038 mmol) was dissolved in DCM (1 mL) at room temperture and TFA (0.8 mL) was added. The reaction mixture was stirred at room temperature for 3 h. The volatiles were evaporated to give Intermediate 140, used without further purification.

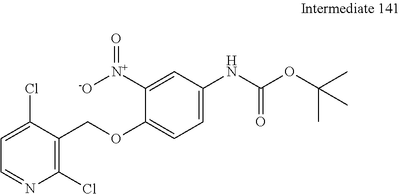

DIAD ([CAS: 2446-83-5], 3.4 mL, 16.8 mmol, 1.2 eq.) was added dropwise to a mixture of 2,4-dichloro-3-pyridinemethanol [CAS: 945543-24-8] (2.49 g, 14.0 mmol, 1.0 eq.), tert-butyl (4-hydroxy-3-nitrophenyl)carbamate ([CAS: 197442-80-1], 3.56 g, 14.0 mmol, 1.0 eq.), Triphenylphosphine [CAS: 603-35-0], 4.41 g, 16.8 mmol, 1.2 eq.) in 2-Methyltetrahydrofuran (50 mL) at room temperature under nitrogen atmosphere. The reaction mixture was stirred overnight. The reaction mixture was diluted with H₂O and extracted with EtOAc. The organic layer was dried over MgSO₄, filtered, and evaporated until dryness. The residue was purified by preparative column chromatography (430 g of 35-40 μm SiOH GraceResolv, gradient from 100% DCM to 97% DCM 3% CH₃OH) to yield Intermediate 141 (4.6 g, yield: 80%).

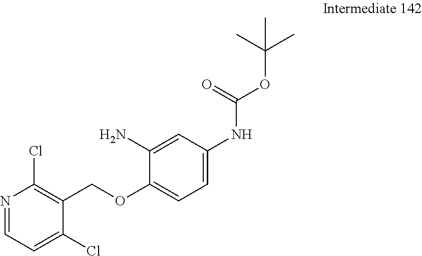 [SMILES: CC(C)(C)OC(=O)Nc1ccc(OCc2c(Cl)ccnc2Cl)c(N)c1]

Iron powder (1.35 g, 24.1 mmol, 5.0 eq.) was added to a solution of Intermediate 141 (2 g, 4.83 mmol) and ammonium chloride (2.58 g, 48.3 mmol, 10.0 eq.) in THF/MeOH/Water (2/2/1, 127 mL), and the reaction mixture was stirred at 80° C. for 3 h. The reaction mixture was cooled to room temperature, poured into a mixture of 10% aqueous K₂CO₃ and DCM, then filtered through a pad of celite. The organic layer was decanted, washed with water, dried over MgSO₄, filtered, and evaporated to yield Intermediate 142 (1.8 g yield: 99%).

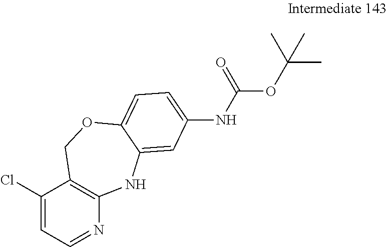 [SMILES: CC(C)(C)OC(=O)Nc1ccc2c(c1)Nc1nccc(Cl)c1CO2]

A solution of Intermediate 142 (1.84 g, 4.78 mmol), Xantphos (166 mg, 0.287 mmol, 0.06 eq.), Pd₂(dba)₃ (131 mg, 0.143 mmol, 0.03 eq.) and Na₂CO₃ (1.01 g, 9.55 mmol, 2.0 eq.) in a mixture of 1,4-dioxane (13.7 mL) and water (1.5 mL) was degassed by bubbling nitrogen gas. The reaction mixture was then stirred at 110° C. for 4 h. The mixture was poured into ice and extracted with EtOAc. The organic layer was washed with brine, dried over MgSO₄, filtered, and the solvent was evaporated. The residue was purified by column chromatography over silica gel (200 g, 15-40 μm, eluent: heptane/EtOAc: 100/0 to 1/100) to yield Intermediate 143 (1.33 g yield: 80%).

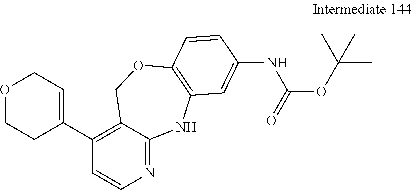 [SMILES: CC(C)(C)OC(=O)Nc1ccc2c(c1)Nc1nccc(C3=CCOCC3)c1CO2]

In a sealed vessel, a solution of Intermediate 143 (805 mg, 2.32 mmol), 2-(3,6-dihydro-2H-pyran-4-yl)-4,4,5,5-tetramethyl-1,3,2-dioxaborolane ([CAS: 287944-16-5], 1.07 g, 5.09 mmol, 2.2 eq.) and potassium phosphate (0.983 g, 4.63 mmol, 2.0 eq.) in 1,4-dioxane (16 mL) and water (2.3 mL) was degassed under nitrogen atmosphere. Pd₂(dba)₃ (212 mg, 0.231 mmol, 0.1 eq.) was added, the reaction mixture was degassed again under nitrogen atmosphere and heated at 100° C. for 2 h. The reaction mixture was cooled to room temperature, poured into water and extracted with EtOAc. The organic layer was washed with water then brine, dried over MgSO₄, filtered over Celite®, and evaporated. The residue was purified by chromatography over silica gel (15-40 μm; 70 g, eluent: heptane/EtOAc: 100/0 to 0/100) to yield Intermediate 144 (734 mg, yield: 80%).

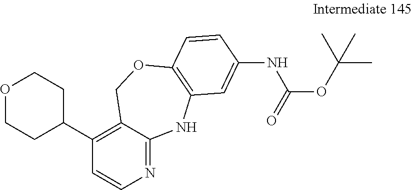 [SMILES: CC(C)(C)OC(=O)Nc1ccc2c(c1)Nc1nccc(C3CCOCC3)c1CO2]

A solution of Intermediate 144 (702 mg, 1.78 mmol) and Pd/C (10%, 349 mg, 0.33 mmol, 0.19 eq.) in MeOH (32 mL) and EtOAc (32 mL) was hydrogenated at room temperature under 2 bars of H₂ for 1 h. The mixture was filtered over celite and evaporated to yield Intermediate 145 (706 mg, yield: 100%) used without further purification.

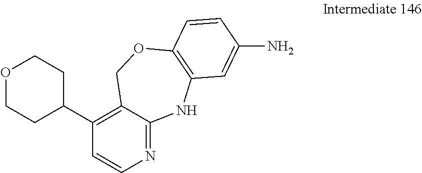 [SMILES: Nc1ccc2c(c1)Nc1nccc(C3CCOCC3)c1CO2]

TFA (3.26 mL, 42.6 mmol, 26.0 eq.) was added at 0° C. to a suspension of Intermediate 145 (0.651 g, 1.64 mmol) in DCM (6.5 mL) and the reaction mixture was stirred at room temperature for 3 h. The reaction mixture was poured onto a 10% aqueous solution of K₂CO₃ and extracted with DCM. The organic layer was decanted, dried over MgSO₄, filtered, and evaporated to yield Intermediate 146 (675 mg, yield: 100%) used without further purification.

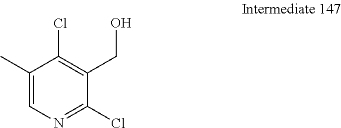

NaBH₄ [CAS: 16940-66-2], 0.624 g, 15.8 mmol, 1.0 eq.) was added portionwise to 2,4-dichloro-5-methylnicotinaldehyde ([CAS: 2369720-14-7], 3.0 g, 15.8 mmol) in MeOH (56 mL) and the reaction mixture was stirred at room temperature 2 h. The reaction mixture was diluted with water and extracted with DCM. The combined organic layer was treated by brine, dried over MgSO₄, filtered, and then evaporated. The residue was purified by column chromatography over silica gel (150 g, 15-40 μm; eluent: DCM/MeOH: 100/0 to 0/100) to yield Intermediate 147 (2.3 g, yield: 75%).

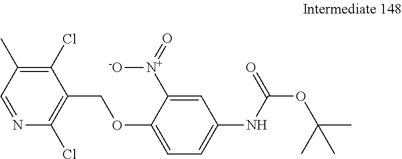

Intermediate 148 was synthesized in a similar manner as Intermediate 141 using Intermediate 147 instead of 2,4-dichloro-3-pyridinemethanol.

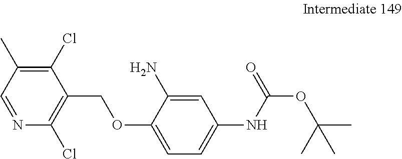 [SMILES: Cc1cnc(Cl)c(COc2ccc(NC(=O)OC(C)(C)C)cc2N)c1Cl]

Intermediate 149 was synthesized in a similar manner as Intermediate 142 using Intermediate 148 instead of Intermediate 141.

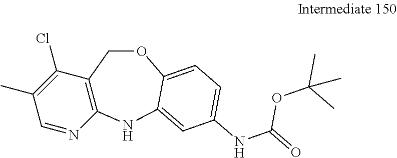 [SMILES: Cc1cnc2c(c1Cl)COc1ccc(NC(=O)OC(C)(C)C)cc1N2]

Intermediate 150 was synthesized in a similar manner as Intermediate 143 using Intermediate 149 instead of Intermediate 142.

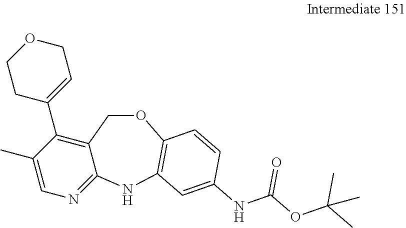 [SMILES: Cc1cnc2c(c1C1=CCOCC1)COc1ccc(NC(=O)OC(C)(C)C)cc1N2]

Intermediate 151 was synthesized in a similar manner as Intermediate 144 using Intermediate 150 instead of Intermediate 143.

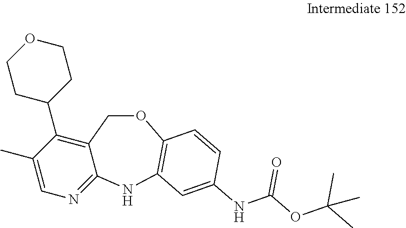 [SMILES: Cc1cnc2c(c1C1CCOCC1)COc1ccc(NC(=O)OC(C)(C)C)cc1N2]

Intermediate 152 was synthesized in a similar manner as Intermediate 145 using Intermediate 151 instead of Intermediate 144.

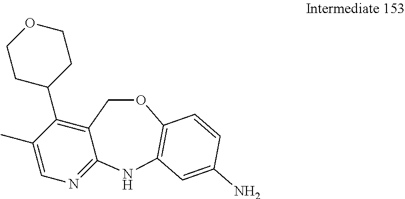 [SMILES: Cc1cnc2c(c1C1CCOCC1)COc1ccc(N)cc1N2]

Intermediate 153 was synthesized in a similar manner as Intermediate 146 using Intermediate 152 instead of Intermediate 145.

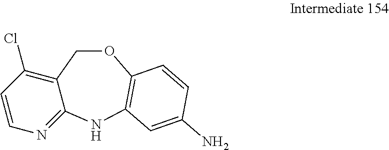 [SMILES: Nc1ccc2c(c1)Nc1nccc(Cl)c1CO2]

HCl (4 M in 1,4-dioxane, 5.25 mL, 21.0 mmol, 14 eq.) was added to Intermediate 143 (521 mg, 1.5 mmol) and the mixture was stirred for 90 min. More HCl (4 M in 1,4-dioxane, 5.25 mL, 21.0 mmol, 14 eq.) was added and the reaction mixture was stirred for 60 min. Then the solvent was evaporated to yield Intermediate 154 (535 mg, quantitative yield) as a white solid.

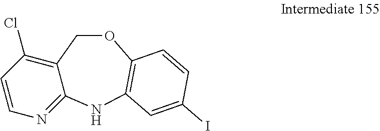 [SMILES: Clc1ccnc2c1COc1ccc(I)cc1N2]

Two solutions, Intermediate 154 (426 mg, 1.5 mmol) and HCl (37% in H₂O, 400 μL, 4.8 mmol, 3.2 eq.) in distilled water (5.6 mL), and sodium nitrite (124 mg, 1.8 mmol, 1.2 eq.) in distilled water (6 mL) were flowed through a LTF MicroShip mixer (0.2 mL) at 0.4 mL/min for each solution (residence time 15 s). The output was collected in a solution of sodium iodide (1.1 g, 7.5 mmol, 5.0 eq.) in EtOAc (20 mL) at 0° C. This mixture was stirred for 45 min at 0° C. The aqueous solution was extracted (3 times) with EtOAc, the organic layers were separated, combined, dried (Na₂SO₄), filtered, and the solvent was evaporated. The residue was purified by column chromatography (silica, EtOAc in heptane 0/100 to 50/50) to yield Intermediate 155 (336 mg, yield: 55%) as a white solid.

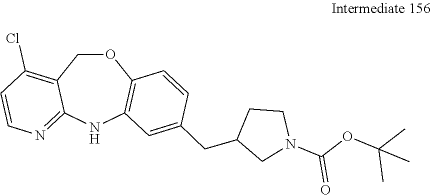 [SMILES: CC(C)(C)OC(=O)N1CCC(Cc2ccc3c(c2)Nc2nccc(Cl)c2CO3)C1]

[[1-[(1,1-Dimethylethoxy)carbonyl]-3-pyrrolidinyl]methyl]iodozine ([CAS: 2135683-48-4], 0.28 M in THF, 5 mL, 1.4 mmol, 1.7 eq.) was added to a mixture of Intermediate 155 (284 mg, 0.8 mmol), Pd(OAc)₂ (8.9 mg, 0.04 mmol, 0.05 eq.), and RuPhos (37 mg, 0.08 mmol, 0.1 eq.) under nitrogen atmosphere. The mixture was stirred at room temperature for 2 h and then at 50° C. for 2 h 30. [[1-[(1,1-Dimethylethoxy)carbonyl]-3-pyrrolidinyl]methyl]iodozine ([CAS: 2135683-48-4], 0.28 M in THF, 5.5 mL, 1.8 mmol, 2.2 eq.) was added again to the mixture at room temperature and the solution was stirred for 3 h. The reaction mixture was diluted with H₂O and a few drops of 32% aqueous NH₃. The reaction mixture was extracted with EtOAc, the organic layer was separated, dried (Na₂SO₄), filtered, and the solvent was evaporated. The residue was purified by column chromatography (silica, EtOAc in DCM 0/100 to 70/30) to yield Intermediate 156 (223 mg, yield: 63%) as a brownish oil.

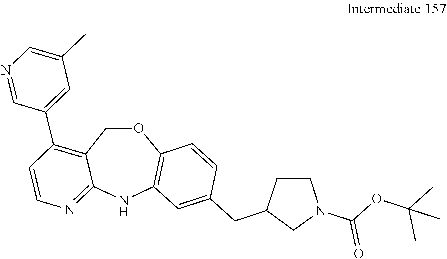 [SMILES: Cc1cncc(-c2ccnc3c2COc2ccc(CC4CCN(C(=O)OC(C)(C)C)C4)cc2N3)c1]

Pd(PPh₃)₄ (42 mg, 0.04 mmol, 0.1 eq.) was added to a stirred suspension of Intermediate 156 (165 mg, 0.39 mmol), 5-methylpyridine-3-boronic acid (76 mg, 0.65 mmol, 1.5 eq.) and saturated aqueous NaHCO₃ (1 mL) in 1,4-dioxane (2 mL) previously purged by bubbling nitrogen during 10 min in a sealed tube. The mixture was heated at 150° C. for 30 min under microwave irradiation. More 5-methylpyridine-3-boronic acid (76 mg, 0.65 mmol, 1.5 eq.) and Pd(PPh₃)₄ (42 mg, 0.04 mmol, 0.1 eq.) were added and the reaction mixture was stirred at 150° C. for 20 min under microwave irradiation. The reaction mixture was diluted with water and extracted with EtOAc. The organic layer was separated, dried (Na₂SO₄), filtered, and the solvent evaporated in vacuo. The residue was purified by flash column chromatography (SiO₂, EtOAc in heptane 0/100 to 0/100) to yield Intermediate 157 (79 mg, yield: 33%) as a yellowish oil.

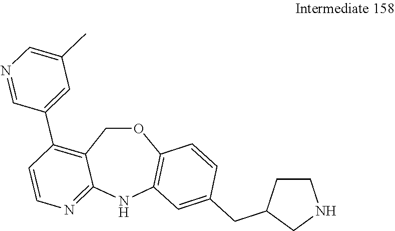 [SMILES: Cc1cncc(-c2ccnc3c2COc2ccc(CC4CCNC4)cc2N3)c1]

HCl (4 M in dioxane, 0.74 mL, 3.0 mmol, 33 eq.) was added to Intermediate 157 (58 mg, 0.09 mmol) and the mixture was stirred at room temperature for 1 h. The solvent was evaporated to yield Intermediate 158 (50 mg, quantitative yield) as a yellow solid which was used in the next step without further purification.

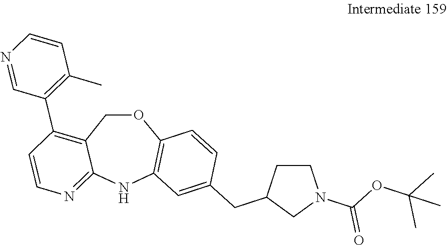 [SMILES: Cc1ccncc1-c1ccnc2c1COc1ccc(CC3CCN(C(=O)OC(C)(C)C)C3)cc1N2]

K₂CO₃ (0.5 mL) was added to a mixture of Intermediate 156 (48 mg, 0.11 mmol), PdCl₂(dppf) (CAS [72287-26-43], 4 mg, 0.005 mmol, 0.05 eq.), (4-methylpyridin-3-yl)boronic acid ([CAS: 148546-82-1], 29 mg, 0.22 mmol, 2 eq.) in 1,4-dioxane. The reaction mixture was degassed with nitrogen and stirred at 150° C. for 10 min under microwave irradiation. The reaction mixture was diluted with water extracted with EtOAc. The organic layer was separated, dried (Na₂SO₄), filtered, and the solvent evaporated. The residue was purified by column chromatography (silica, EtOAc in DCM 0/100 to 100/0) to yield Intermediate 159 (27 mg, yield: 53%) as a yellow oil.

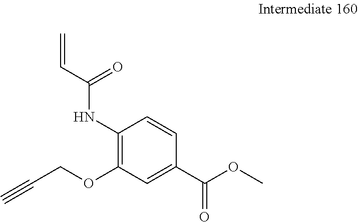 [SMILES: C#CCOc1cc(C(=O)OC)ccc1NC(=O)C=C]

Acryloyl chloride (44 μL, 0.54 mmol, 1.1 eq.) was added to a stirred solution of methyl 4-amino-3-(prop-2-yn-1-yloxy)benzoate ([CAS: 1621429-33-1], 100 mg, 0.49 mmol) and Et₃N (203 μL, 1.5 mmol, 3 eq.) in DCM (7 mL) at 0° C. The reaction mixture was stirred at 0° C. for 15 min. The reaction mixture was diluted with DCM and water. The organic layer was separated, dried (Na₂SO₄), filtered, and concentrated in vacuo. The residue was purified by flash column chromatography (silica; EtOAc in heptane 0/100 to 100/0) to yield Intermediate 160 (80 mg, yield: 51%) as a yellow solid.

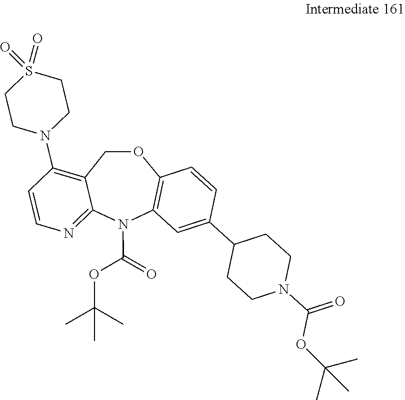 [SMILES: CC(C)(C)OC(=O)N1CCC(c2ccc3c(c2)N(C(=O)OC(C)(C)C)c2nccc(N4CCS(=O)(=O)CC4)c2CO3)CC1]

Pd₂(dba)₃ ([CAS: 51364-51-3], 53.2 mg, 0.058 mmol, 0.03 eq.) and S-Phos ([CAS: 657408-07-6], 48 mg, 0.12 mmol, 0.06 eq.) were added to a solution of Intermediate 11 (1.0 g, 1.9 mmol), thiomorpholine 1,1-dioxide ([CAS: 39093-93-1], 314 mg, 2.3 mmol, 1.2 eq.), and Cs₂CO₃ (2.7 g, 8.1 mmol, 4.2 eq.) in 1,4-dioxane (13 mL) under nitrogen atmosphere and the reaction mixture was stirred at 100° C. for 2 h 30 min. The reaction mixture was diluted with a 10% K₂CO₃ aqueous solution and extracted with DCM. The organic layer was washed with brine, dried over MgSO₄, filtered, and concentrated. The residue was purified by column chromatography (Irregular SiOH 15-40 μm 80 g GraceResolv, gradient from 100% DCM to 93% DCM, 7% MeOH, 0.7% NH₄OH) to give Intermediate 161 (262 mg, yield: 22%).

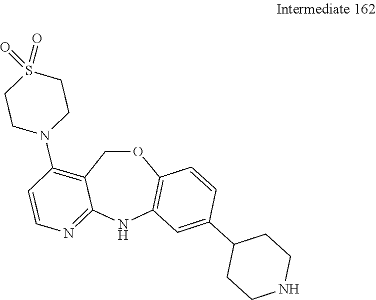 [SMILES: O=S1(=O)CCN(c2ccnc3c2COc2ccc(C4CCNCC4)cc2N3)CC1]

TFA (5.6 mL, 73.1 mmol, 171 eq,) was added to a stirred solution of Intermediate 161 (262 mg, 0.426 mmol) in DCM (10.6 mL) at room temperature and the reaction mixture was stirred at room temperature for 12 h. The reaction mixture was concentrated. Water and NH₄OH were added until a basic pH was reached. The mixture was extracted twice with DCM. The organic layer was dried over MgSO₄, filtered, and evaporated to give Intermediate 162 (190 mg, quantitative yield).

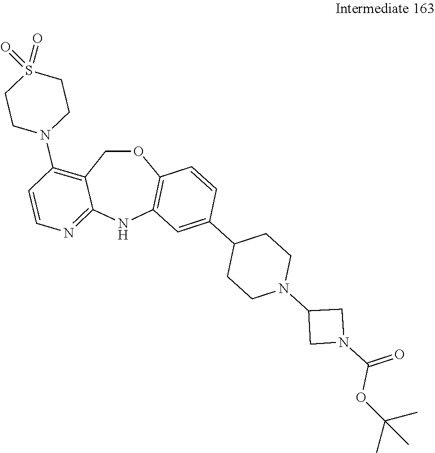 [SMILES: CC(C)(C)OC(=O)N1CC(N2CCC(c3ccc4c(c3)Nc3nccc(N5CCS(=O)(=O)CC5)c3CO4)CC2)C1]

A solution of Intermediate 162 (187 mg, 0.451 mmol), 1-Boc-3-azetidinone ([CAS: 398489-26-4], 116 mg, 0.68 mmol, 1.5 eq.), AcOH (47 μL, 0.81 mmol, 1.8 eq.), and sodium triacetoxyborohydride ([CAS: 56553-60-7], 191 mg, 0.90 mmol, 2.0 eq.) in DCM (1.3 mL) was stirred at room temperature for 4 h. The reaction was quenched with a K₂CO₃ 10% aqueous solution and extracted with DCM. The organic layer was washed with brine, dried over MgSO₄, filtered, and the solvent was evaporated. The residue was purified by chromatography over silica gel (SiO₂, Grace 40 g, eluent: from 97% DCM, 3% MeOH, 0.3% NH₄OH to 90% DCM, 10% MeOH, 1% NH₄OH) to give Intermediate 163 (125 mg, yield 49%).

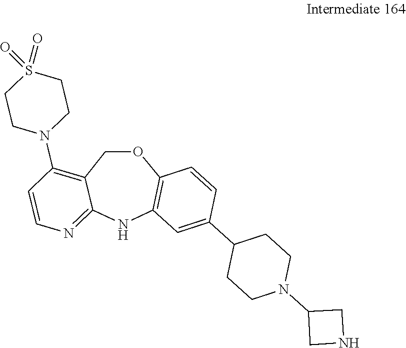 [SMILES: O=S1(=O)CCN(c2ccnc3c2COc2ccc(C4CCN(C5CNC5)CC4)cc2N3)CC1]

HCl (4M in dioxane, 549 μL, 4 M, 2.2 mmol, 10 eq.) was added to a solution of Intermediate 163 (125 mg, 0.22 mmol) in 1,4-dioxane (1.5 mL) and MeOH (0.9 mL) and the reaction mixture was stirred at room temperature for 12 h. The reaction mixture was concentrated and co-evaporated with DCM to give Intermediate 164 (123 mg, quantitative yield).

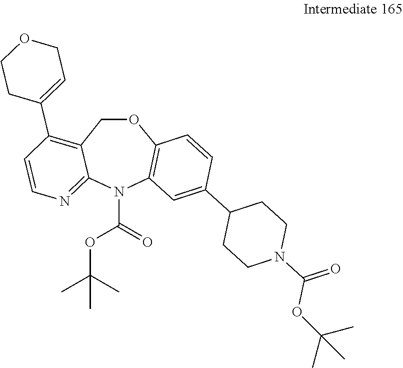 [SMILES: CC(C)(C)OC(=O)N1CCC(c2ccc3c(c2)N(C(=O)OC(C)(C)C)c2nccc(C4=CCOCC4)c2CO3)CC1]

In a sealed vessel, a solution of Intermediate 11 (330 mg, 0.639 mmol), 3,6-dihydro-2H-pyran-4-boronic acid pinacol ester ([CAS: 287944-16-5], 296 mg, 1.409 mmol, 2.2 eq.), potassium phosphate (272 mg, 1.281 mmol, 2.0 eq.), Pd₂(dba)₃ (59 mg, 0.0644 mmol, 0.1 eq.) and tricyclohexylphosphine ([CAS: 2622-14-2], 43 mg, 0.153 mmol, 0.24 eq.) in 1,4-dioxane (4.9 mL) and water (0.6 mL) was degassed under nitrogen atmosphere and heated at 100° C. for 2 h. After cooling, water was added and this mixture was extracted twice with EtOAc. The combined organic layer was evaporated. The residue was purified by column chromatography (Irregular SiOH 15-40 μm 40 g GraceResolv®, gradient from 90% heptane, 10% EtOAc to 20% heptane, 80% EtOAc) to give Intermediate 165 (295 mg, yield: 82%)

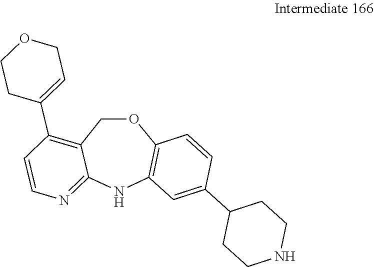 [SMILES: C1=C(c2ccnc3c2COc2ccc(C4CCNCC4)cc2N3)CCOC1]

TFA (1.6 mL, 20.9 mmol, 40.6 eq.) was added to a mixture of Intermediate 165 (290 mg, 0.51 mmol) in DCM (10 mL) at 0° C. and the reaction mixture was stirred at room temperature overnight. A mixture of DCM/MeOH/NH₄OH and water was added. The reaction mixture was stirred at room temperature for 10 min. The organic layer was separated and the solvent was evaporated to give Intermediate 166 (293 mg, quantitative yield). This product was used without further purification.

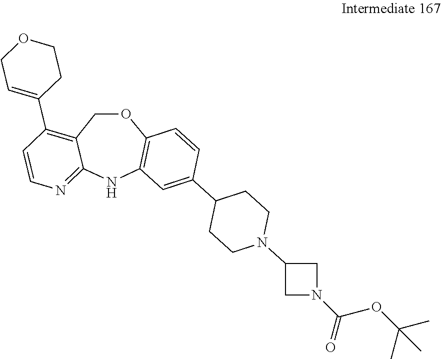 [SMILES: CC(C)(C)OC(=O)N1CC(N2CCC(c3ccc4c(c3)Nc3nccc(C5=CCOCC5)c3CO4)CC2)C1]

Intermediate 167 was synthesized in a similar manner as Intermediate 163 using Intermediate 166 instead of Intermediate 162.

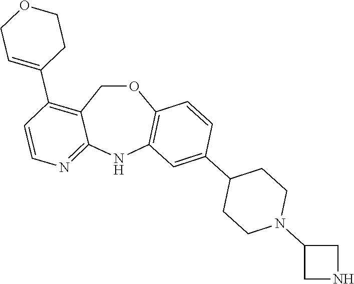 [SMILES: C1=C(c2ccnc3c2COc2ccc(C4CCN(C5CNC5)CC4)cc2N3)CCOC1]

TFA (1 mL, 13.1 mmol, 19.4 eq.) was added to a solution of Intermediate 167 (349 mg, 0.673 mmol) in DCM (2 mL) at 0° C. The reaction mixture was stirred at room temperature overnight. The solvent was evaporated and HCl (3 N water, 2.00 mL, 3 M, 6.0 mmol, 8.9 eq.) was added. The solvent was evaporated. More HCl (3 M in H₂O, 1 mL, 3.0 mmol, 4.5 eq.) was added and the solvent was evaporated to give Intermediate 168 (239 mg, 78%).

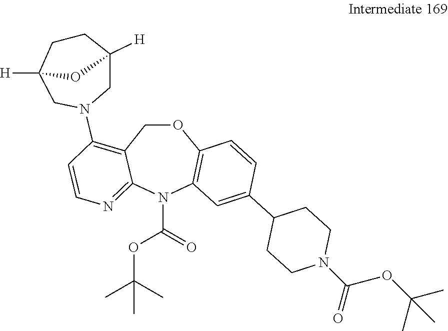 [SMILES: CC(C)(C)OC(=O)N1CCC(c2ccc3c(c2)N(C(=O)OC(C)(C)C)c2nccc(N4C[C@H]5CC[C@@H](C4)O5)c2CO3)CC1]

A solution of Intermediate 11 (1 g, 1.938 mmol), 8-oxa-3-azabicyclo[3.2.1]octane ([CAS: 39093-93-1], 263 mg, 2.3 mmol, 1.2 eq.), Pd(OAc)₂ ([CAS: 3375-31-3], 506 mg, 0.19 mmol, 0.1 eq.), rac-BINAP ([CAS: 98327-87-8], 241 mg, 0.39 mmol, 0.2 eq.), and cesium carbonate (1578 g, 4.8 mmol, 2.5 eq.) in DMF (10 mL) was purged with nitrogen and the reaction mixture was stirred at 100° C. for 2 h. The reaction mixture was poured into water and DCM and filtered over Celite©. The organic layer was separated, dried over MgSO₄, filtered, and concentrated. The residue was purified by column chromatography (Stationary phase: irregular SiOH 15-40 μm 80 g GRACE, gradient from 100% DCM to 97% DCM, 3% MeOH with 2% NH₄OH) to yield Intermediate 169 (1 g, yield: 87%).

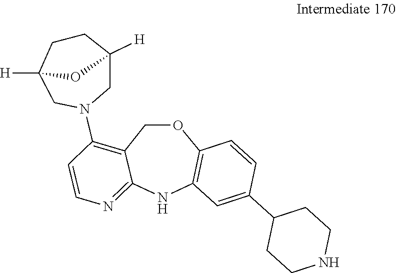 [SMILES: c1cc(N2C[C@H]3CC[C@@H](C2)O3)c2c(n1)Nc1cc(C3CCNCC3)ccc1OC2]

Intermediate 170 was synthesized in a similar manner as Intermediate 162 using Intermediate 169 instead of Intermediate 161.

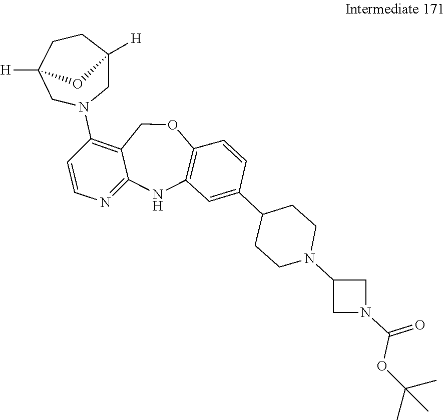 [SMILES: CC(C)(C)OC(=O)N1CC(N2CCC(c3ccc4c(c3)Nc3nccc(N5C[C@H]6CC[C@@H](C5)O6)c3CO4)CC2)C1]

Intermediate 171 was synthesized in a similar manner as Intermediate 163 using Intermediate 170 instead of Intermediate 162.

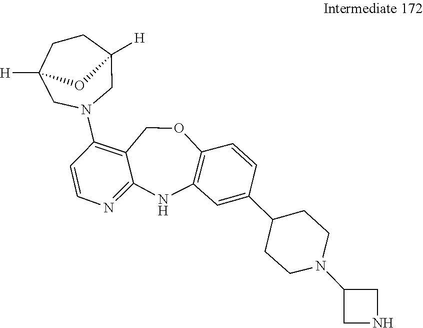 [SMILES: c1cc(N2C[C@H]3CC[C@@H](C2)O3)c2c(n1)Nc1cc(C3CCN(C4CNC4)CC3)ccc1OC2]

Intermediate 172 was synthesized in a similar manner as Intermediate 164 using Intermediate 171 instead of Intermediate 163.

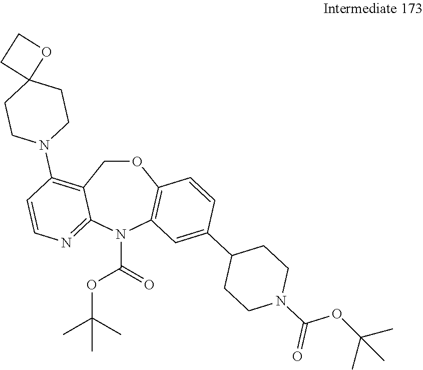 [SMILES: CC(C)(C)OC(=O)N1CCC(c2ccc3c(c2)N(C(=O)OC(C)(C)C)c2nccc(N4CCC5(CCO5)CC4)c2CO3)CC1]

Intermediate 173 was synthesized in a similar manner as Intermediate 169 using 1-oxa-7-azaspiro[3.5]nonane [CAS: 38674-21-4] instead of 8-oxa-3-azabicyclo[3.2.1]octane [CAS: 39093-93-1].

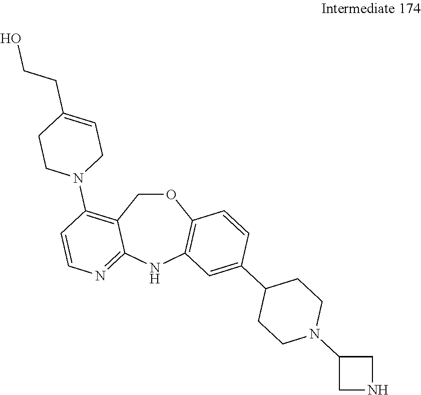 [SMILES: OCCC1=CCN(c2ccnc3c2COc2ccc(C4CCN(C5CNC5)CC4)cc2N3)CC1]

Intermediate 174 was synthesized following the synthetic route of Intermediate 162 to Intermediate 164 starting with Intermediate 173 instead of Intermediate 161. The oxetane ring opened to the hydroxyethyl group during the Boc deprotection step.

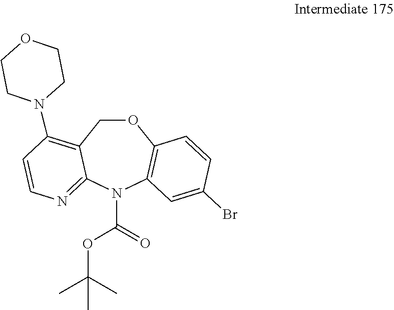 [SMILES: CC(C)(C)OC(=O)N1c2cc(Br)ccc2OCc2c(N3CCOCC3)ccnc21]

A solution of Intermediate 4 (5.0 g, 12.1 mmol), NiCl₂ glyme ([CAS: 29046-78-4], 182 mg, 0.83 mmol, 0.07 eq.), DABCO (2.36 g, 21.0 mmol, 1.7 eq.), [Ir{dF(CF₃)ppy}2(dtbpy)]PF₆ ([CAS: 870987-63-6] (182 mg, 0.162 mmol, 0.01 eq.) and morpholine (3.3 mL, 38.3 mmol, 3.2 eq.) in DMA (85 mL) was degassed by sparging nitrogen. The reaction mixture was stirred at room temperature for 2 days under LED irradiation (royal blue LED, 6 cm from the reaction mixture). The reaction mixture was diluted with water and saturated aqueous solution of NH₄Cl and extracted twice with EtOAc. The combined organic layer was evaporated. The solid was washed twice with Et₂O then dried to give Intermediate 175 (2.93 g, yield: 52%).

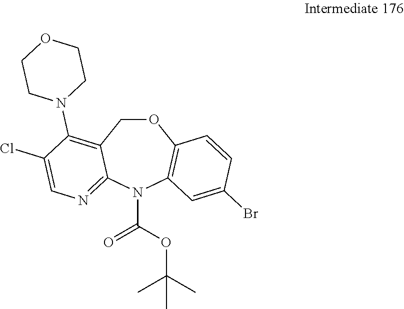 [SMILES: CC(C)(C)OC(=O)N1c2cc(Br)ccc2OCc2c1ncc(Cl)c2N1CCOCC1]

Intermediate 175 (980 mg, 2.12 mmol) and N-chlorosuccinimide ([CAS: 128-09-6, (708 mg, 5.3 mmol, 2.5 eq.) in DMF (16 mL) were stirred at room temperature for 7 h. The reaction mixture was diluted with water and the precipitate was filtered off. The filtrate was extracted with DCM. The organic layer was dried over MgSO₄, filtered, and concentrated. The residue was purified by column chromatography (irregular SiOH 15-40 μm 12 g Grace, gradient from 90% heptane, 10% EtOAc to 50% heptane, 50% EtOAc) to yield Intermediate 176 (1080 mg, yield: 68%).

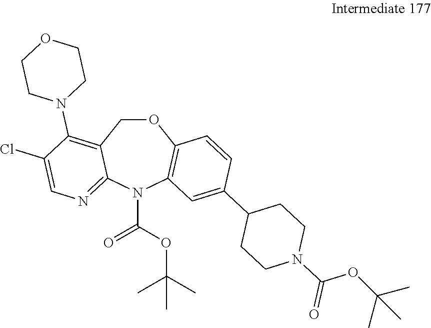 [SMILES: CC(C)(C)OC(=O)N1CCC(c2ccc3c(c2)N(C(=O)OC(C)(C)C)c2ncc(Cl)c(N4CCOCC4)c2CO3)CC1]

[1-[(1,1-Dimethylethoxy)carbonyl]-4-piperidinyl]iodozine ([CAS: 807618-13-9], 8.2 mL, 0.55 M, 4.5 mmol, 2.2 eq.), Intermediate 176 (1 g, 2.0 mmol), 1,1′-bis(diphenylphosphino)ferrocene-palladium(II)dichloride DCM adduct (166 mg, 0.2 mmol, 0.1 eq.), and copper(I)iodide ([CAS: 7681-65-4], 50 mg, 0.26 mmol, 0.13 eq.) in DMA (8 mL) in a sealed tube were stirred at 80° C. under microwave irradiation for 65 min. The reaction mixture was poured into 10% aqueous NH₄Cl. DCM was added and the mixture was filtered over Celite©. The filtrate was decanted, the organic layer was separated, dried over MgSO₄, filtered, and evaporated. The residue was purified by chromatography (SiO₂, 40 g; eluent: from 90% heptane, 10% EtOAc to 50% heptane, 50% EtOAc) to yield Intermediate 177 (728 mg, yield 60%).

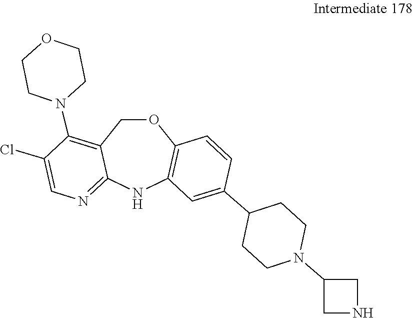 [SMILES: Clc1cnc2c(c1N1CCOCC1)COc1ccc(C3CCN(C4CNC4)CC3)cc1N2]

Intermediate 178 was synthesized following the synthetic route of Intermediate 162 to Intermediate 164 starting with Intermediate 177 instead of Intermediate 161.

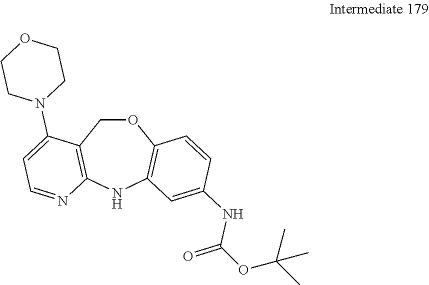 [SMILES: CC(C)(C)OC(=O)Nc1ccc2c(c1)Nc1nccc(N3CCOCC3)c1CO2]

A solution of Intermediate 143 (3 g, 8.6 mmol), morpholine (1.13 g, 12.9 mmol, 1.5 eq.), and DABCO (1.94 g, 17.3 mmol, 2.0 eq.) in dry dimethylacetamide (10 mL) was degassed with nitrogen. NiCl₂ glyme ([CAS: 29046-78-4], 95 mg, 0.43 mmol, 0.05 eq.) and (Ir[dF(CF₃)ppy]₂(dtbpy))PF₆ ([CAS: 870987-63-6], 10 mg, 0.009 mmol, 0.001 eq.) were added. The mixture was stirred under blue LED irradiation without fan cooling, at ˜55° C., for 4 days. Aqueous NaHCO₃ was added and the reaction mixture was extracted with EtOAc. The organic layer was washed with brine, dried over MgSO₄, filtered, and evaporated to dryness. The residue was purified by flash column chromatography on silica gel (gradient of EtOAc-heptane 30% to 100%) to afford Intermediate 179 (1.11 g. yield: 32%).

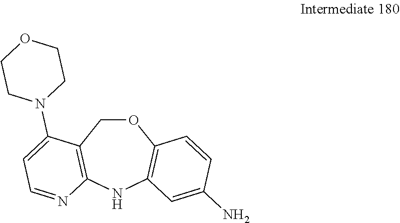 [SMILES: Nc1ccc2c(c1)Nc1nccc(N3CCOCC3)c1CO2]

TFA (6 mL) was added to a solution of Intermediate 179 (1.11 g, 2.8 mmol) in DCM (40 mL) and the mixture was stirred for 3 h at room temperature. The mixture was concentrated under reduced pressure. The residue was diluted with EtOAc and water and basified with saturated aqueous Na₂CO₃. The organic layer was separated, dried with MgSO₄, filtered, and concentrated to yield Intermediate 180 (1.15 g, quantitative yield).

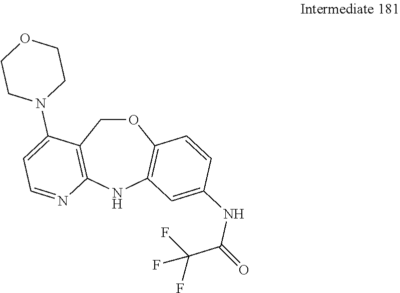 [SMILES: O=C(Nc1ccc2c(c1)Nc1nccc(N3CCOCC3)c1CO2)C(F)(F)F]

Trifluoroacetic anhydride (775 μL, 5.6 mmol, 2.0 eq.) was added to a solution of Intermediate 180 (1.15 g, 2.8 mmol) in DCM (20 mL). The mixture was cooled in a ice bath, Et₃N (1.9 mL, 13.9 mmol, 5.0 eq.) was added and the reaction mixture was stirred at room temperature for 1 h. The reaction mixture was quenched by addition of saturated aqueous NaHCO₃. and extracted with DCM. The organic layer was dried over anhydrous MgSO₄, filtered, and concentrated under vacuum. The residue was purified by flash column chromatography on silica gel (heptane—EtOAc gradient) to yield Intermediate 181 (1.35 g, quantitative yield).

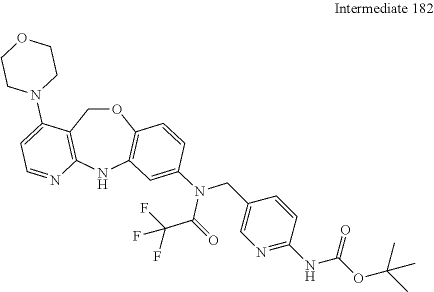 [SMILES: CC(C)(C)OC(=O)Nc1ccc(CN(C(=O)C(F)(F)F)c2ccc3c(c2)Nc2nccc(N4CCOCC4)c2CO3)cn1]

Potassium carbonate (578 mg, 4.2 mmol, 2.0 eq.) was added to a solution of Intermediate 181 (824 mg, 2.1 mmol) and 1,1-dimethylethyl N-[5-(bromomethyl)-2-pyridinyl]carbamate ([CAS: 304873-96-9], 600 mg, 2.1 mmol) in acetone (5 mL) and the mixture was stirred at room temperature for 15 h. The reaction mixture was poured into water/NaHCO₃ and extracted with EtOAc. The organic layer was washed with brine, dried over MgSO₄, filtered, and concentrated. Purification by column flash chromatography (SiO₂, EtOAc-heptane gradient) yielded Intermediate 182 (1.3 g, quantitative yield).

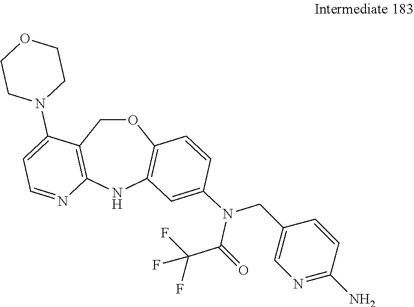 [SMILES: Nc1ccc(CN(C(=O)C(F)(F)F)c2ccc3c(c2)Nc2nccc(N4CCOCC4)c2CO3)cn1]

TFA (2 mL) was added to a solution of Intermediate 182 (1.3 g, 2.1 mmol) in DCM (10 mL) and the mixture was stirred at room temperature for 5 h. The mixture was evaporated and the residue was taken up with DCM and poured into water/NaHCO₃. The layers were separated. The organic layer was washed with brine, dried over MgSO₄, filtered, and concentrated to dryness. Purification by column flash chromatography (SiO₂, MeOH-DCM gradient) yielded Intermediate 183 (626 mg, yield: 60%).

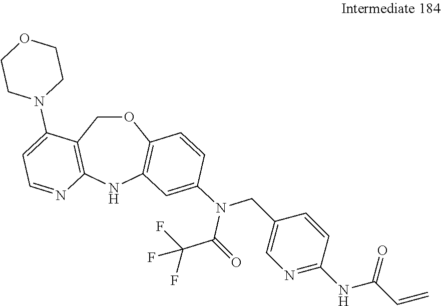 [SMILES: C=CC(=O)Nc1ccc(CN(C(=O)C(F)(F)F)c2ccc3c(c2)Nc2nccc(N4CCOCC4)c2CO3)cn1]

Triethylamine (251 μL, 1.8 mmol, 3.0 eq.) was added to a solution of Intermediate 183 (300 mg, 0.6 mmol) in DCM (4 mL). The mixture was cooled in an ice bath and acryloyl chloride (58 μL, 0.7 mmol, 1.2 eq.) was added dropwise. The reaction mixture was stirred at room temperature for 3 h. The reaction mixture was quenched by addition of saturated aqueous NaHCO₃ and extracted with DCM. The organic layer was dried over anhydrous MgSO₄ and concentrated under vacuum. The residue was purified by flash column chromatography (SiO₂, EtOAc-heptane gradient) followed by another flash column chromatography (SiO₂, MeOH-DCM gradient) to yield Intermediate 184 (65 mg, yield: 20%).

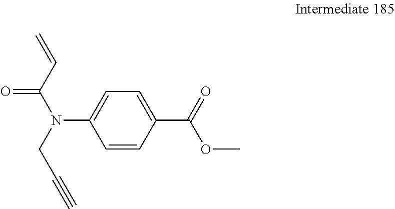 [SMILES: C#CCN(C(=O)C=C)c1ccc(C(=O)OC)cc1]

Acryloyl chloride (380 μL, 4.7 mmol, 2.0 eq.) was added to a stirred solution of methyl 4-(2-propyn-1-ylamino)benzoate ([CAS: 1218756-64-9], 443 mg, 2.3 mmol) and Et₃N (976 μL, 7.0 mmol, 3.0 eq.) in DCM (35 mL) at 0° C. The mixture was stirred at 0° C. for 2 h. The mixture was diluted with water and extracted with DCM. The organic layer was separated, dried (Na₂SO₄), filtered, and concentrated in vacuo. The residue was purified by flash column chromatography (silica; EtOAc in heptane 0/100 to 30/70) to yield Intermediate 185 (220 mg, yield: 39%) as a yellow solid.

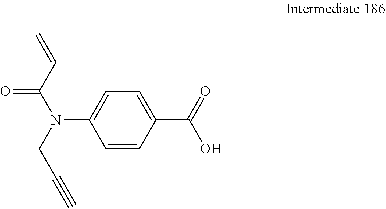 [SMILES: C#CCN(C(=O)C=C)c1ccc(C(=O)O)cc1]

LiOH (5 mg, 0.21 mmol) was added to a solution of Intermediate 185 (50 mg, 0.21 mmol) in THF (0.5 mL) and water (0.1 mL) and the reaction mixture was stirred at room temperature for 16 h. The reaction mixture was concentrated in vacuo to yield Intermediate 186 (48 mg, yield: 99%) as a yellow solid.

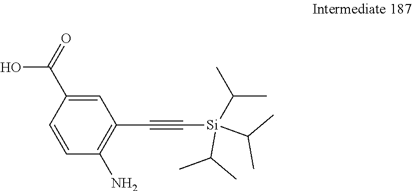 [SMILES: CC(C)[Si](C#Cc1cc(C(=O)O)ccc1N)(C(C)C)C(C)C]

A solution of 4-bromo-5-iodopyridin-2-amine ([CAS: 1186115-39-8], 3 g, 11.4 mmol), (triisopropylsilyl)acetylene ([CAS: 89343-06-6], 3.12 g, 17.1 mmol, 1.5 eq.), and bis(triphenylphosphine)palladium(II) chloride ([CAS: 13965-03-2], 800 mg, 1.14 mmol, 0.1 eq.) in Et₃N (30 mL) and DMF (30 mL) was stirred at 20° C. The reaction mixture was degassed by evacuating and backfilling with nitrogen via a needle. Cuprous iodide ([CAS: 7681-65-4], 218 mg, 1.14 mmol, 0.1 eq.) was added under nitrogen atmosphere. The mixture was stirred at 80° C. overnight. The reaction was quenched with water (30 mL) and the mixture was extracted with EtOAc (100 mL). The organic layer was washed with brine (20 mL) and dried over Na₂SO₄, and concentrated in vacuo. The residue was purified by flash column chromatography on silica gel (petroleum ether/EtOAc from I/O to 1/1) to give Intermediate 187 (2.6 g, yield: 70%) as a yellow solid.

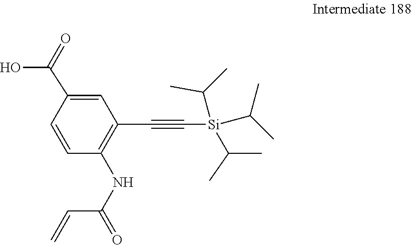 [SMILES: C=CC(=O)Nc1ccc(C(=O)O)cc1C#C[Si](C(C)C)(C(C)C)C(C)C]

Saturated aqueous NaHCO₃ (6 mL) was added dropwise to a solution of Intermediate 187 (1.2 g, 3.7 mmol) in THF at room temperature. Acrylic anhydride (559 mg, 4.4 mmol, 1.2 eq.) was added dropwise at 0° C. The mixture was stirred at 20° C. for 2 h. The reaction was quenched with water (30 mL) and extracted with EtOAc (100 mL×2). The combined organic layer was washed with brine (20 mL), dried over Na₂SO₄, and concentrated in vacuo. The residue was purified by flash silica gel chromatography (12 g SepaFlash® Silica Flash Column, gradient from 0 to 60% EtOAc/petroleum ether) to provide a white solid. A second purification was performed by preparative HPLC (column: Boston Uni C18 40*150 mm*5 um; gradient: water (0.225% FA)-ACN; B %: from 65% to 95%) to afford Intermediate 188 (496 mg, yield: 35%) as a white solid.

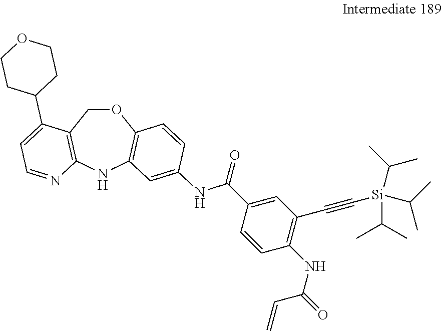 [SMILES: C=CC(=O)Nc1ccc(C(=O)Nc2ccc3c(c2)Nc2nccc(C4CCOCC4)c2CO3)cc1C#C[Si](C(C)C)(C(C)C)C(C)C]

Intermediate 188 (409 mg, 1.07 mmol, 008 eq.), DIPEA (2.4 mL, 13.4 mmol, 10.0 eq.), and HATU (763 mg, 2.0 mmol, 1.5 eq.) were added to a solution of Intermediate 146 (550 mg, 1.34 mmol) in DMF (10 mL) at 20° C. and the solution was stirred at 20° C. for 3 h. The reaction mixture was concentrated in vacuum. The residue was purified by flash column chromatography on silica gel (eluent: petroleum ether: EtOAc 100:0 to 0:100) to give the Intermediate 189 (850 mg, yield: 88%) as a yellow solid.

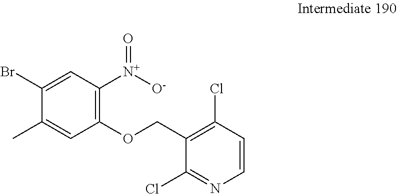 [SMILES: Cc1cc(OCc2c(Cl)ccnc2Cl)c([N+](=O)[O-])cc1Br]

2,4-Dichloro-3-pyridinemethanol ([CAS: 945543-24-8], 0.8 g, 4.4 mol), 4-bromo-5-methyl-2-nitrophenol ([CAS: 182500-28-3], 1.02 g, 4.4 mmol) and triphenylphosphine (2.31 g, 8.8 mmol, 2.0 eq.) were mixed in dry THF (30 mL) under nitrogen atmosphere. DIAD (1.7 mL, 8.8 mmol, 2.0 eq.) was added dropwise and the reaction mixture was stirred at room temperature for 2 h. The reaction mixture was diluted with DCM and SiO₂ was added. Solvents were evaporated and the residue was loaded into a refillable column and purified by column flash chromatography (silica; heptane (10% DCM)/EtOAc from 5% EtOAc to 70% EtOAc) to afford Intermediate 190 (1.7 g, quantitative yield).

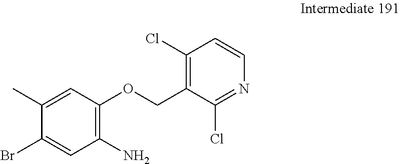 [SMILES: Cc1cc(OCc2c(Cl)ccnc2Cl)c(N)cc1Br]

Intermediate 190 (1.76 g, 4.4 mmol), iron (2.48 g, 44.0 mmol, 10.0 eq.) and glacial acetic acid (5.0 mL, 88.1 mmol, 20.0 eq.) were stirred in MeOH (50 mL) at room temperature for 1 h. The reaction mixture was diluted with EtOAc. Ice and saturated aqueous NaHCO₃ were added slowly until a basic pH was reached. The combined organic layers were dried (MgSO₄), filtered, and concentrated to yield Intermediate 191 (1.6 g, quantitative yield), used without further purification.

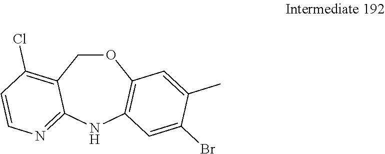 [SMILES: Cc1cc2c(cc1Br)Nc1nccc(Cl)c1CO2]

Intermediate 191 (1.6 g, 4.4 mmol) and TFA (1.0 mL, 13.2 mmol, 3.0 eq.) were dissolved in 1,4-dioxane and the reaction mixture was stirred at 120° C. for 8 h. The reaction mixture was cooled to room temperature and concentrated. The residue was diluted with DCM, washed with aqueous NaHCO₃ and brine. The organic layer was dried (MgSO₄) and concentrated to yield Intermediate 192 (1.4 g, quantitative yield).

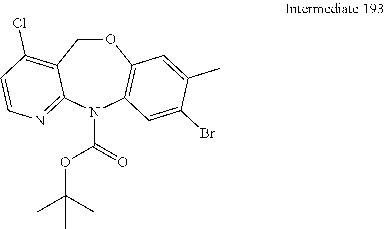 [SMILES: Cc1cc2c(cc1Br)N(C(=O)OC(C)(C)C)c1nccc(Cl)c1CO2]

(Boc)₂O (3.3 g, 15.1 mmol, 3.0 eq.) was added to a solution of Intermediate 192 (1.7 g, 5.0 mmol), DMAP (310 mg, 2.5 mmol, 0.5 eq.) and Et₃N (2.1 mL, 15.1 mmol, 3.0 eq.) in DCM (25 mL) and the mixture was stirred at room temperature for 20 h. The mixture was directly purified by flash column chromatography (SiO₂, EtOAc-heptane gradient 5% to 50%) to afford Intermediate 193 (1.9 g, yield: 90%).

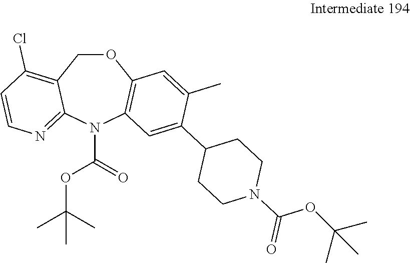 [SMILES: Cc1cc2c(cc1C1CCN(C(=O)OC(C)(C)C)CC1)N(C(=O)OC(C)(C)C)c1nccc(Cl)c1CO2]

Intermediate 193 (1.9 g, 4.5 mmol), 4-bromo-Boc-piperidine ([CAS: 180695-79-89], 1.2 g, 4.5 mmol), Ni(II)Cl₂ glyme ([CAS: 29046-78-4], 98 mg, 0.45 mmol, 0.1 eq.), sodium tetrafluoroborate ([CAS: 13755-29-8], 245 mg, 2.2 mmol, 0.5 eq.), 1,10-phenantroline ([CAS: 66-71-7], 161 mg, 0.9 mmol, 0.2 eq.) Mn powder (325 Mesh CAS: [7439-96-5], 490 mg, 8.9 mmol, 2.0 eq.) and 4-ethylpyridine ([CAS: 536-75-4], 2.54 μL, 2.2 mmol, 0.5 eq.) in MeOH (25 mL) were placed in a screw cap vial under nitrogen atmosphere. The reaction mixture was heated at 60° C. for 20 h. The mixture was cooled to room temperature and diluted with EtOAc. The solids were removed by filtration over celite, and the filtrate was concentrated. Purification was performed by flash column chromatography (SiO₂, EtOAc-heptane gradient) to afford Intermediate 194 (220 mg, yield: 9%).

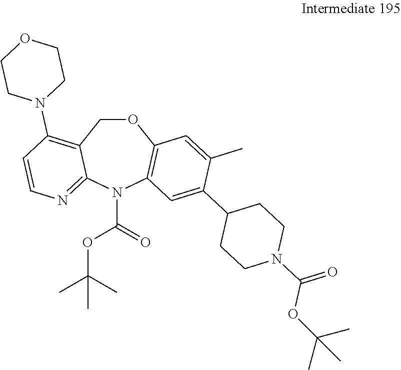 [SMILES: Cc1cc2c(cc1C1CCN(C(=O)OC(C)(C)C)CC1)N(C(=O)OC(C)(C)C)c1nccc(N3CCOCC3)c1CO2]

Intermediate 195 was synthesized in a similar manner as Intermediate 179 using Intermediate 194 instead of Intermediate 143.

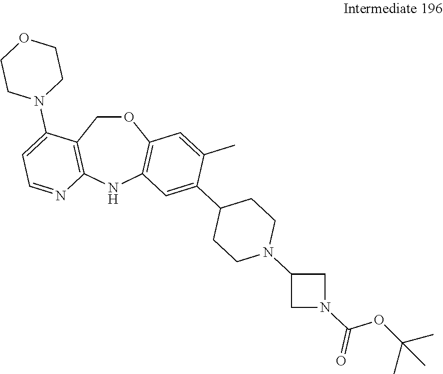 [SMILES: Cc1cc2c(cc1C1CCN(C3CN(C(=O)OC(C)(C)C)C3)CC1)Nc1nccc(N3CCOCC3)c1CO2]

Intermediate 196 was synthesized following the synthetic route from Intermediate 162 to Intermediate 163 starting with Intermediate 195 instead of Intermediate 161.

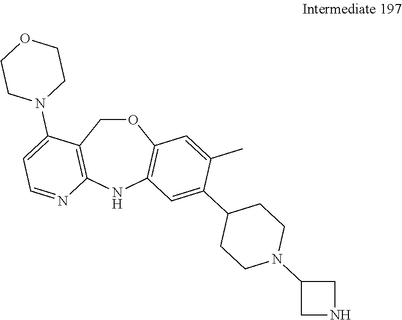 [SMILES: Cc1cc2c(cc1C1CCN(C3CNC3)CC1)Nc1nccc(N3CCOCC3)c1CO2]

TFA (1 mL) was added to a solution of Intermediate 196 (74 mg, 0.14 mmol) in DCM, and the reaction mixture was stirred at room temperature for 3 h. The mixture was evaporated to dryness, taken up with DCM, poured into water/K₂CO₃, and extracted with DCM. The organic layer was washed with brine, dried over MgSO₄, filtered, and concentrated to yield Intermediate 197 (61 mg, quantitative yield).

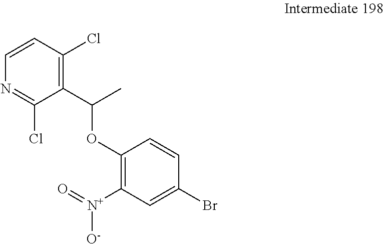 [SMILES: CC(Oc1ccc(Br)cc1[N+](=O)[O-])c1c(Cl)ccnc1Cl]

1-(2,4-Dichloropyridin-3-yl)ethan-1-ol ([CAS: 1246349-88-1], 1.14 g, 5.9 mmol), 4-bromo-2-nitrophenol ([CAS: 7693-52-9], 1.3 g, 5.9 mmol), and triphenylphosphine (4.6 g, 17.6 mmol, 3.0 eq.) were mixed in dry THF (70 mL) under nitrogen atmosphere. DIAD (3.5 mL, 17.6 mmol, 3.0 eq.) was added dropwise and the reaction mixture was stirred at room temperature for 1 h. The reaction mixture was partitioned between EtOAc and brine. The combined organic layers were dried (MgSO₄), filtered, and concentrated. The residue was purified by flash column chromatography (silica; DCM/EtOAc from 100/0 to 60/40) to afford Intermediate 198 (2.1 g, yield: 84%) as a yellowish solid.

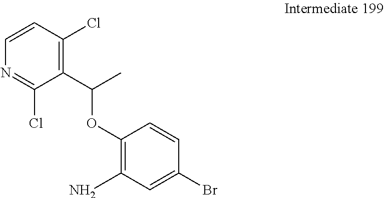 [SMILES: CC(Oc1ccc(Br)cc1N)c1c(Cl)ccnc1Cl]

Intermediate 198 (1.6 g, 4.0 mmol), iron (2.3 g, 40.0 mmol, 10.0 eq.), and glacial acetic acid (4.6 mL, 80.0 mmol, 20.0 eq.) were stirred in MeOH (20 mL) at 80° C. for 2 h. The reaction mixture was diluted with EtOAc. Then, saturated NaHCO₃ solution was added slowly until a basic pH was reached. The combined organic layers were dried (MgSO₄), filtered, and concentrated. To avoid the presence of acetic acid the residue was co-evaporated twice with toluene to afford Intermediate 199 (1.5 g, quantitative yield).

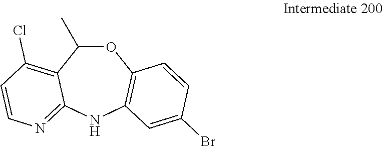 [SMILES: CC1Oc2ccc(Br)cc2Nc2nccc(Cl)c21]

Intermediate 200 was synthesized in a similar manner as Intermediate 3 using Intermediate 199 instead of Intermediate 2.

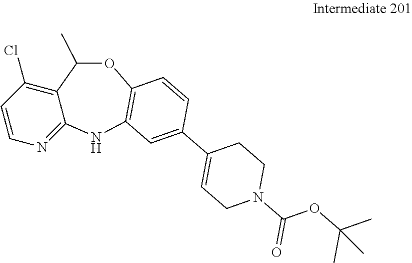 [SMILES: CC1Oc2ccc(C3=CCN(C(=O)OC(C)(C)C)CC3)cc2Nc2nccc(Cl)c21]

Intermediate 201 was synthesized in a similar manner as Intermediate 20 using Intermediate 200 instead of Intermediate 19.

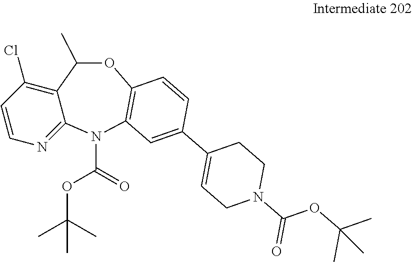 [SMILES: CC1Oc2ccc(C3=CCN(C(=O)OC(C)(C)C)CC3)cc2N(C(=O)OC(C)(C)C)c2nccc(Cl)c21]

(Boc)₂O (2.8 g, 13.0 mmol, 6.0 eq.) was added to a solution of Intermediate 201 (1 g, 2.2 mmol) and DMAP (134 mg, 1.1 mmol, 0.5 eq.) in DCM (10 mL) and the reaction mixture was stirred at room temperature for 16 h. (Boc)₂O (1.9 g, 8.7 mmol, 4.0 eq.) was added again and the reaction mixture was stirred for 2 h. The reaction mixture was partitioned between EtOAc and brine. The combined organic layers were dried (MgSO₄), filtered, and concentrated. The residue was purified by flash column chromatography (silica; heptane/EtOAc from 100/0 to 50/50) to afford Intermediate 202 (1.06 g, yield: 86%) as a yellow solid.

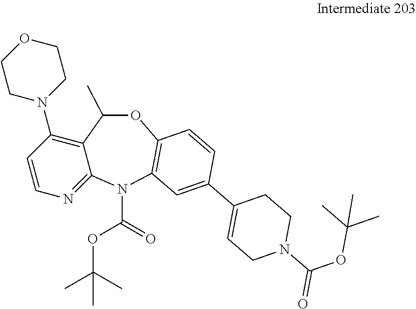 [SMILES: CC1Oc2ccc(C3=CCN(C(=O)OC(C)(C)C)CC3)cc2N(C(=O)OC(C)(C)C)c2nccc(N3CCOCC3)c21]

Intermediate 202 (1.05 g, 1.8 mmol), morpholine (244 mg, 2.8 mmol, 1.5 eq.), and DABCO (428 mg, 3.7 mmol, 2.0 eq.) were dissolved in dry DMA (8 mL) and degassed with nitrogen. NiCl₂ glyme ([CAS: 29046-78-4], 40.5 mg, 0.19 mmol, 0.1 eq.) and (Ir[dF(CF₃)ppy]₂(dtbpy)) ([CAS: 870987-63-6], 4 mg, 0.004 mmol, 0.002 eq.) were added and the mixture was degassed for 1 min. The reaction mixture was stirred under blue LED irradiation without fan cooling for 16 h. NiCl₂ glyme ([CAS: 29046-78-4], 40.5 mg, 0.19 mmol, 0.1 eq.) and (Ir[dF(CF₃)ppy]2(dtbpy)) ([CAS: 870987-63-6], 4 mg, 0.004 mmol, 0.002 eq.) were added again. The reaction mixture was degassed and stirred under blue LED irradiation without fan cooling for 60 h. The reaction mixture was partitioned between EtOAc and saturated aqueous NaHCO₃. The combined organic layers were dried (MgSO₄), filtered, and concentrated. The residue was purified by flash column chromatography (Silica; heptane/EtOAc from 100/0 to 40/60) to afford Intermediate 203 (513 mg, yield: 47%) as a fluorescent yellow solid.

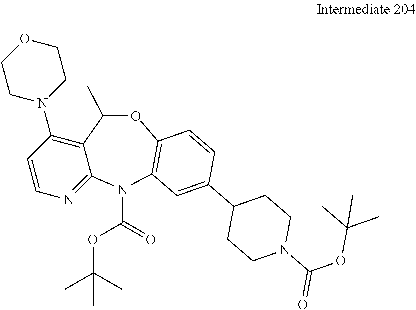 [SMILES: CC1Oc2ccc(C3CCN(C(=O)OC(C)(C)C)CC3)cc2N(C(=O)OC(C)(C)C)c2nccc(N3CCOCC3)c21]

A solution of Intermediate 203 (347 mg, 0.6 mmol) in MeOH (10 mL) and THF (10 mL) was stirred at room temperature under H₂ atmosphere for 4 h. The reaction mixture was filtered through a pad of celite and the solvents removed under vacuo to afford Intermediate 204 (356 mg, quantitative yield), used without further purification.

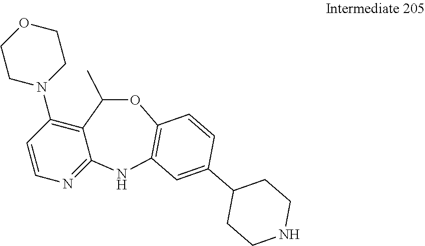 [SMILES: CC1Oc2ccc(C3CCNCC3)cc2Nc2nccc(N3CCOCC3)c21]

TFA (3.6 mL) was added to a solution of Intermediate 204 (524 mg, 0.9 mmol) in DCM (6 mL) and the reaction mixture was stirred at room temperature for 4 h. The solvents were removed in vacuo. Excess TFA was removed co-evaporating with xylene twice. The residue was dissolved in DCM and basified with aqueous NaHCO₃ until basic pH. The layers were separated and the combined organic layers were dried (MgSO₄), filtered, and concentrated to yield Intermediate 205 (326 mg, quantitative yield), used without further purification.

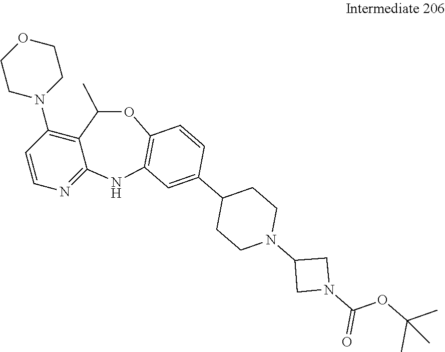 [SMILES: CC1Oc2ccc(C3CCN(C4CN(C(=O)OC(C)(C)C)C4)CC3)cc2Nc2nccc(N3CCOCC3)c21]

Intermediate 205 (325 mg, 0.9 mmol), tert-butyl-3-oxoazetidine-1-carboxylate (219 mg, 1.3 mmol, 1.5 eq.), and glacial acetic acid (59 μL, 1.0 mmol, 1.2 eq.) were dissolved in MeOH (20 mL) and the reaction mixture was stirred at room temperature for 1 h. Sodium cyanoborohydride (54 mg, 0.9 mmol, 1.0 eq.) was added and the reaction mixture was stirred at room temperature for 16 h. tert-Butyl-3-oxoazetidine-1-carboxylate (73 mg, 0.45 mmol, 0.5 eq.) was added again and the reaction mixture was stirred for 1 h before sodium cyanoborohydride (27 mg, 0.45 mmol, 0.5 eq.) was added. The reaction mixture was stirred at room temperature for 60 h. The reaction mixture was partitioned between EtOAc and saturated NaHCO₃ solution. The combined organic layers were dried (MgSO₄), filtered, and concentrated. The residue was purified by flash column chromatography (silica; heptane/EtOAc from 100/0 to 0/100) to afford Intermediate 206 (210 mg, yield: 44%) as a fluorescent yellow solid.

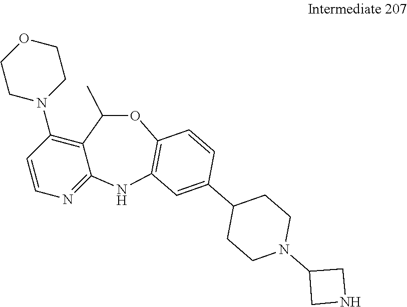 [SMILES: CC1Oc2ccc(C3CCN(C4CNC4)CC3)cc2Nc2nccc(N3CCOCC3)c21]

TFA (1.6 mL) was added to a solution of Intermediate 206 (190 mg, 0.4 mmol) in DCM (2.4 mL) and the reaction mixture was stirred at room temperature for 6 h. The solvents were removed in vacuo. The residue was partitioned between EtOAc and saturated NaHCO₃ solution. The aqueous layer was extracted twice using DCM/MeOH (9/1). The combined organic layers were dried (MgSO₄), filtered, and concentrated to afford Intermediate 207 (90 mg, yield: 58%).

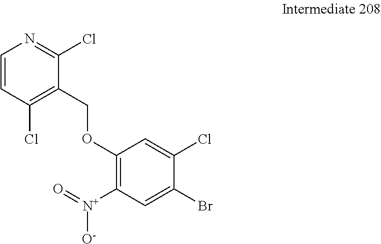 [SMILES: O=[N+]([O-])c1cc(Br)c(Cl)cc1OCc1c(Cl)ccnc1Cl]

Intermediate 208 was synthesized in a similar manner as Intermediate 1 using 4-bromo-5-chloro-2-nitrophenol [CAS: 65001-78-7] instead of 4-bromo-2-nitrophenol.

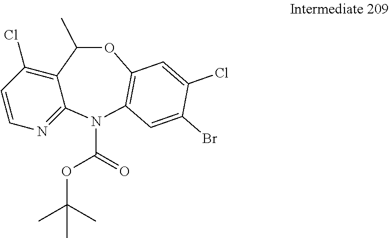 [SMILES: CC1Oc2cc(Cl)c(Br)cc2N(C(=O)OC(C)(C)C)c2nccc(Cl)c21]

Intermediate 209 was synthesized following the synthetic route from Intermediate 17 to Intermediate 19 starting with Intermediate 208 instead of Intermediate 16.

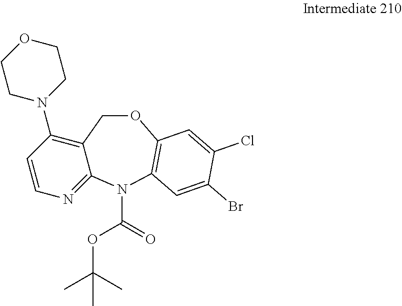 [SMILES: CC(C)(C)OC(=O)N1c2cc(Br)c(Cl)cc2OCc2c(N3CCOCC3)ccnc21]

Intermediate 210 was synthesized in a similar manner as Intermediate 179 using Intermediate 209 instead of Intermediate 143.

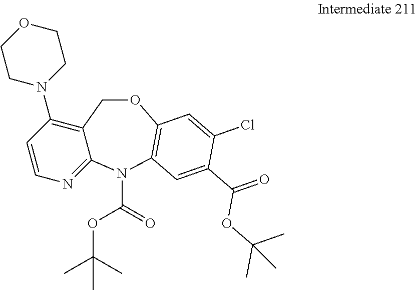 [SMILES: CC(C)(C)OC(=O)c1cc2c(cc1Cl)OCc1c(N3CCOCC3)ccnc1N2C(=O)OC(C)(C)C]

[1-[(1,1-Dimethylethoxy)carbonyl]-4-piperidinyl]iodozine ([CAS: 807618-13-9], crude solution in DMA, equivalent to 443 mg, 1.18 mmol, 1.4 eq.) was added to a solution of Intermediate 210 (417 mg, 0.84 mmol), Pd(dppf)Cl₂ ([CAS: 72287-26-4], 21 mg, 0.025 mmol, 0.03 eq.), and CuI ([CAS: 7681-65-4], 10 mg, 0.05 mmol, 0.06 eq.) in dry DMA (3 mL) under nitrogen atmosphere at room temperature. The reaction mixture was stirred under nitrogen atmosphere at 80° C. overnight. The reaction mixture was cooled to room temperature, diluted with EtOAc, and washed with saturated aqueous NaHCO₃ and with brine. The combined organic layer was dried (MgSO₄), filtered, and concentrated. The residue was purified by column flash chromatography on silica gel (hexane/EtOAc) to yield Intermediate 211 (260 mg, yield: 52%).

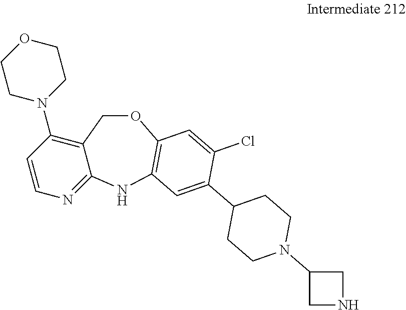 [SMILES: Clc1cc2c(cc1C1CCN(C3CNC3)CC1)Nc1nccc(N3CCOCC3)c1CO2]

Intermediate 212 was synthesized following the synthetic route of Intermediate 162 to Intermediate 164 starting with Intermediate 211 instead of Intermediate 161.

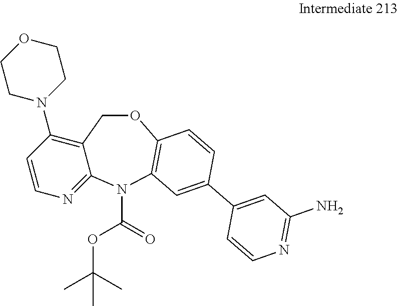 [SMILES: CC(C)(C)OC(=O)N1c2cc(-c3ccnc(N)c3)ccc2OCc2c(N3CCOCC3)ccnc21]

Intermediate 213 was synthesized in a similar manner as Intermediate 20 using Intermediate 175 instead of Intermediate 19 and 2-aminopyridine-4-boronic acid, pinacol ester [CAS: 1195995-72-2] instead of N-Boc-1,2,3,6-tetrahydropyridine-4-boronic acid pinacol ester.

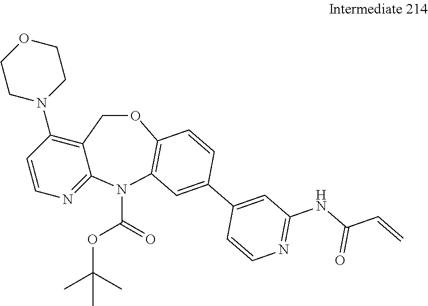 [SMILES: C=CC(=O)Nc1cc(-c2ccc3c(c2)N(C(=O)OC(C)(C)C)c2nccc(N4CCOCC4)c2CO3)ccn1]

Et₃N (215 μL, 1.542 mmol, 5 eq.) was added to a solution of Intermediate 213 (217 mg, 0.308 mmol) in DCM (4 mL). The reaction mixture was cooled in an ice bath and a solution of acryloyl chloride (25 μL, 0.308 mmol, 1 eq.) in DCM (1 mL) was added dropwise. The reaction mixture was stirred at room temperature for 6 h. More acryloyl chloride (12 μL, 0.154 mmol, 0.5 eq.) was added and stirring was continued at room temperature overnight. The reaction was quenched by addition of saturated aqueous NaHCO₃ and the mixture was extracted with DCM. The organic layer was dried over MgSO₄, filtered, and concentrated under vacuum. The residue was purified by flash chromatography (SiO₂, MeOH-DCM gradient) to give Intermediate 214 (83 mg, yield: 51%) and unreacted Intermediate 213 (54 mg, yield: 37%).

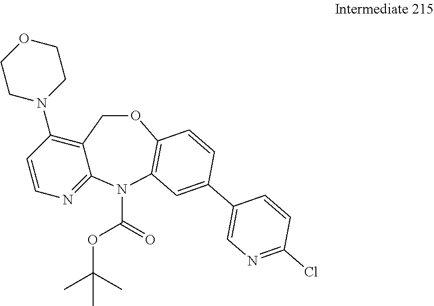 [SMILES: CC(C)(C)OC(=O)N1c2cc(-c3ccc(Cl)nc3)ccc2OCc2c(N3CCOCC3)ccnc21]

Intermediate 215 was synthesized in a similar manner as Intermediate 20 using Intermediate 175 instead of Intermediate 19 and 6-chloro-3-pyridinylboronic acid [CAS: 444120-91-6] instead of N-Boc-1,2,3,6-tetrahydropyridine-4-boronic acid pinacol ester.

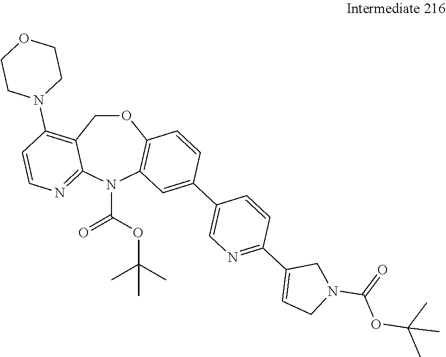 [SMILES: CC(C)(C)OC(=O)N1CC=C(c2ccc(-c3ccc4c(c3)N(C(=O)OC(C)(C)C)c3nccc(N5CCOCC5)c3CO4)cn2)C1]

Intermediate 216 was synthesized in a similar manner as Intermediate 20 using Intermediate 215 instead of Intermediate 19 and tert-butyl 3-(4,4,5,5-tetramethyl-1,3,2-dioxaborolan-2-yl)-2,5-dihydro-1H-pyrrole-1-carboxylate [CAS: 212127-83-8] instead of N-Boc-1,2,3,6-tetrahydropyridine-4-boronic acid pinacol ester.

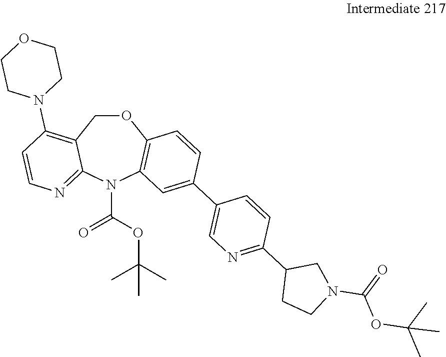 [SMILES: CC(C)(C)OC(=O)N1CCC(c2ccc(-c3ccc4c(c3)N(C(=O)OC(C)(C)C)c3nccc(N5CCOCC5)c3CO4)cn2)C1]

Pd/C 10% (60 mg) was added to a solution of Intermediate 216 (275 mg, 0.44 mmol) in MeOH under nitrogen atmosphere at 0° C., then hydrogen gas was bubbled, and the mixture was stirred for 3 days at room temperature. The mixture was filtered through a pad of celite, and the solvent was removed under reduced pressure to yield Intermediate 217 (238 mg, yield: 86%) used without further purification.

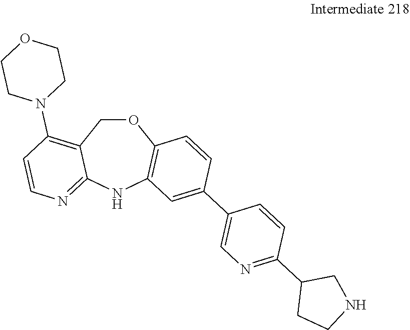 [SMILES: c1cc(N2CCOCC2)c2c(n1)Nc1cc(-c3ccc(C4CCNC4)nc3)ccc1OC2]

HCl (4 N in 1,4-dioxane, 1.9 mL, 7.6 mmol, 20.0 eq.) was added to a solution of Intermediate 217 (238 mg, 0.38 mmol) in DCM (10 mL) and the reaction mixture was stirred overnight at room temperature. The mixture was concentrated to afford Intermediate 218 (188 mg, quantitative yield), used without further purification.

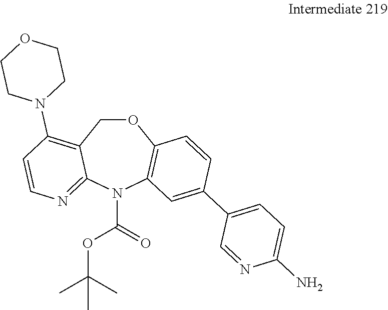 [SMILES: CC(C)(C)OC(=O)N1c2cc(-c3ccc(N)nc3)ccc2OCc2c(N3CCOCC3)ccnc21]

Intermediate 219 was synthesized in a similar manner as Intermediate 20 using Intermediate 175 instead of Intermediate 19 and 2-aminopyridine-5-boronic acid pinacol ester [CAS: 827614-64-2] instead of N-Boc-1,2,3,6-tetrahydropyridine-4-boronic acid pinacol ester.

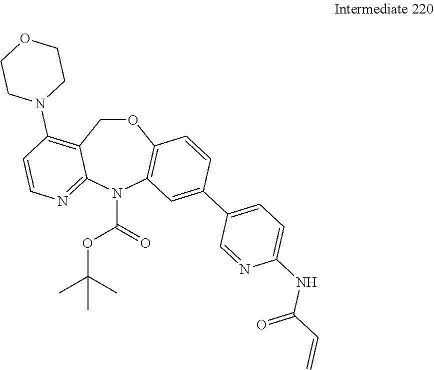 [SMILES: C=CC(=O)Nc1ccc(-c2ccc3c(c2)N(C(=O)OC(C)(C)C)c2nccc(N4CCOCC4)c2CO3)cn1]

Intermediate 220 was synthesized in a similar manner as Intermediate 214 using Intermediate 219 instead of Intermediate 213.

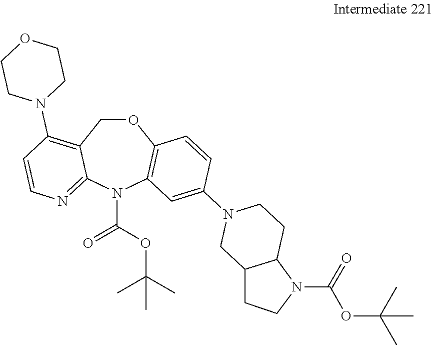 [SMILES: CC(C)(C)OC(=O)N1c2cc(N3CCC4C(CCN4C(=O)OC(C)(C)C)C3)ccc2OCc2c(N3CCOCC3)ccnc21]

Intermediate 175 (350 mg, 0.76 mmol), cesium carbonate (493 mg, 1.5 mmol, 2.0 eq.), DavePhos ([CAS: 213697-53-1], 60 mg, 0.15 mmol, 0.2 eq.), and Pd₂(dba)₃ ([CAS: 51364-51-3], 69 mg, 0.076 mmol, 0.1 eq.) were mixed in 1,4-dioxane (12 mL) under nitrogen atmosphere. The reaction mixture was stirred for 10 min at room temperature. 1,1-Dimethylethyl octahydro-1H-pyrrolo[3,2-c]pyridine-1-carboxylate ([CAS: 1147422-00-1], 198 mg, 0.83 mmol, 1.1 eq.) was added and the reaction mixture was stirred at 100° C. for 6 h. The mixture was cooled to room temperature, diluted with EtOAc and washed with saturated aqueous NaHCO₃ and with brine. The combined organic layer was dried (MgSO₄) and concentrated. The residue was purified by flash column chromatography on silica gel (hexane/EtOAc) to afford Intermediate 221 (410 mg, yield: 89%).

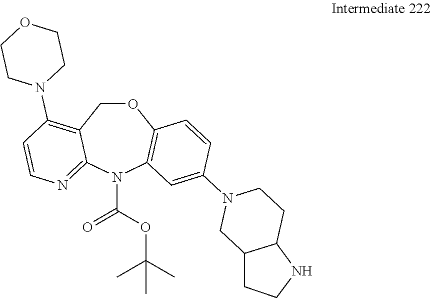 [SMILES: CC(C)(C)OC(=O)N1c2cc(N3CCC4NCCC4C3)ccc2OCc2c(N3CCOCC3)ccnc21]

TFA (4 mL) was added to a solution of Intermediate 221 (410 mg, 0.68 mmol) in DCM (6 mL) and the mixture was stirred at room temperature for 3 h. The reaction mixture was evaporated to afford Intermediate 222 (429 mg, quantitative yield).

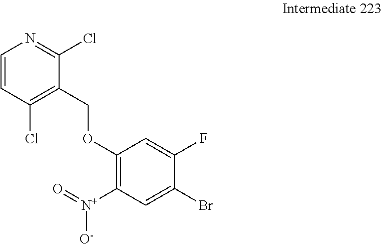 [SMILES: O=[N+]([O-])c1cc(Br)c(F)cc1OCc1c(Cl)ccnc1Cl]

Intermediate 223 was synthesized in a similar manner as Intermediate 31 using 4-bromo-5-fluoro-2-nitrophenol [CAS: 1016234-87-9] instead of Intermediate 30.

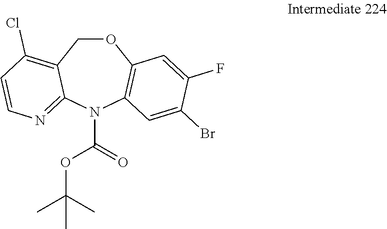 [SMILES: CC(C)(C)OC(=O)N1c2cc(Br)c(F)cc2OCc2c(Cl)ccnc21]

Intermediate 224 was synthesized following the synthetic route from Intermediate 17 to Intermediate 19 starting with Intermediate 223 instead of Intermediate 16.

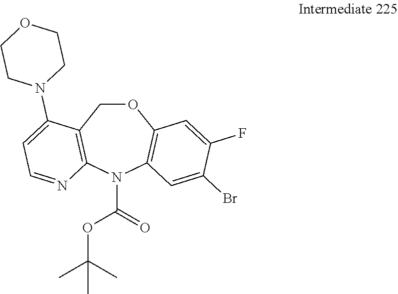 [SMILES: CC(C)(C)OC(=O)N1c2cc(Br)c(F)cc2OCc2c(N3CCOCC3)ccnc21]

Intermediate 225 was synthesized in a similar manner as Intermediate 179 using Intermediate 224 instead of Intermediate 143.

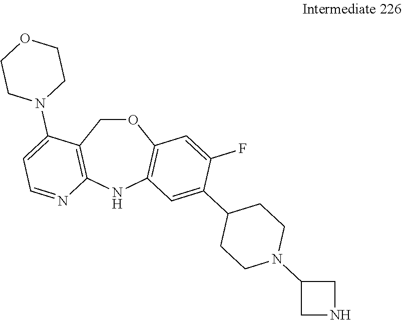 [SMILES: Fc1cc2c(cc1C1CCN(C3CNC3)CC1)Nc1nccc(N3CCOCC3)c1CO2]

Intermediate 226 was synthesized following the synthetic route from Intermediate 211 to Intermediate 212 starting with Intermediate 225 instead of Intermediate 210.

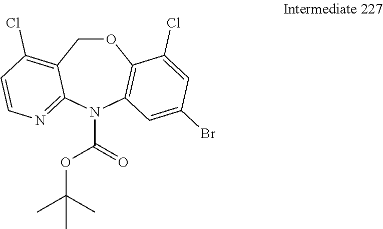 [SMILES: CC(C)(C)OC(=O)N1c2cc(Br)cc(Cl)c2OCc2c(Cl)ccnc21]

Intermediate 227 was synthesized following the synthetic route from Intermediate 16 to Intermediate 19 starting from 4-bromo-2-chloro-6-nitrophenol [CAS: 58349-01-2] instead of 4-bromo-2-methyl-6-nitrophenol.

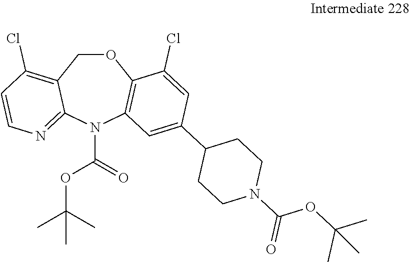 [SMILES: CC(C)(C)OC(=O)N1CCC(c2cc(Cl)c3c(c2)N(C(=O)OC(C)(C)C)c2nccc(Cl)c2CO3)CC1]

Intermediate 228 was synthesized in a similar manner as Intermediate 78 using Intermediate 227 instead of 2-chloro-5-(methoxymethoxy)pyridine.

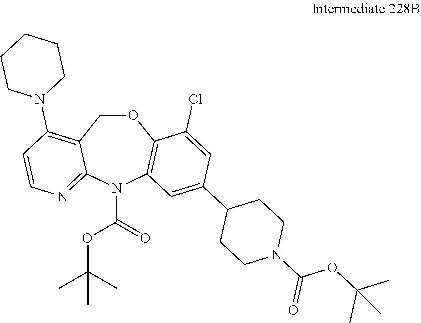 [SMILES: CC(C)(C)OC(=O)N1CCC(c2cc(Cl)c3c(c2)N(C(=O)OC(C)(C)C)c2nccc(N4CCCCC4)c2CO3)CC1]

Intermediate 228B was synthesized in a similar manner as Intermediate 179 using Intermediate 228 instead of Intermediate 143.

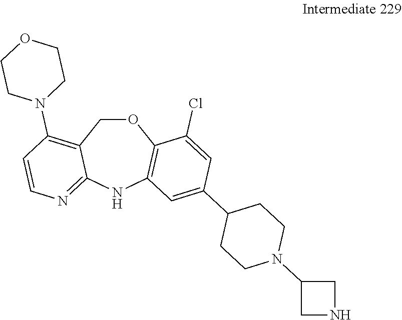 [SMILES: Clc1cc(C2CCN(C3CNC3)CC2)cc2c1OCc1c(N3CCOCC3)ccnc1N2]

Intermediate 229 was synthesized following the synthetic route from Intermediate 162 to Intermediate 164 starting with Intermediate 228B instead of Intermediate 161.

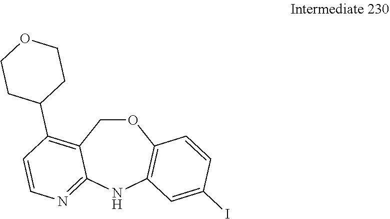 [SMILES: Ic1ccc2c(c1)Nc1nccc(C3CCOCC3)c1CO2]

HCl (37% in H₂O, 1.1 mL, 12.6 mmol, 16.7 eq.) was added dropwise to Intermediate 145 (300 mg, 0.76 mmol) at 0° C. The mixture was stirred for 20 min at 0° C. Then a solution of sodium nitrite (63 mg, 0.91 mmol, 1.2 eq.) in water (7.4 mL) and EtOAc (8.8 mL) was added. The resulting mixture was stirred at 0° C. for 20 min. Sodium iodide (566 mg, 3.8 mmol, 5.0 eq.) was added portionwise and the reaction mixture was stirred at 0° C. for 3 h. The reaction mixture was neutralized with saturated aqueous Na₂CO₃ at 0° C. and it was extracted with EtOAc (×3). The combined organic layers were washed with brine, dried (Na₂SO₄), filtered, and concentrated. The residue was dissolved in DCM, treated with saturated aqueous Na₂CO₃, and the mixture was stirred at room temperature for 1 h. The organic layer was separated, dried (Na₂SO₄), filtered, and concentrated to give Intermediate 230 (255 mg, yield: 83%) as a beige solid.

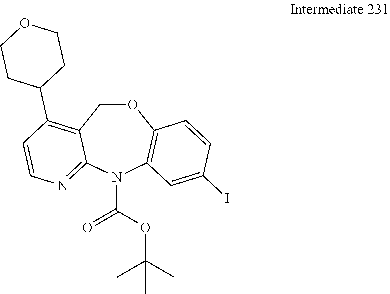 [SMILES: CC(C)(C)OC(=O)N1c2cc(I)ccc2OCc2c(C3CCOCC3)ccnc21]

LHMDS (1.06 M in THF, 1.0 mL, 1.0 mmol, 1.6 eq.) was added to a solution of Intermediate 230 (255 mg, 0.63 mmol) and Boc-anhydride (409 mg, 1.9 mmol, 3.0 eq.) in THF (5.1 mL). The mixture was stirred overnight at room temperature. The excess of base was quenched with 10% aqueous NH₄Cl and the reaction mixture was extracted with EtOAc. The combined organic layer was separated, dried (Na₂SO₄), filtered, and the solvent evaporated. The residue was purified by column chromatography (silica, EtOAc in DCM 0/100 to 30/70) to yield Intermediate 231 (257 mg, yield: 81%) as a foam.

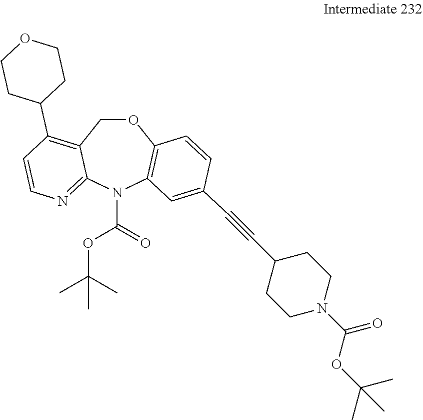 [SMILES: CC(C)(C)OC(=O)N1CCC(C#Cc2ccc3c(c2)N(C(=O)OC(C)(C)C)c2nccc(C4CCOCC4)c2CO3)CC1]

Intermediate 231 (40 mg, 0.079 mmol), 1,1-dimethylethyl 4-ethynyl-1-piperidinecarboxylate ([CAS: 287192-97-6], 25 mg, 0.12 mmol, 1.5 eq.), Pd(PPh₃)₂Cl₂ ([13965-03-2], 3 mg, 0.004 mmol, 0.05 eq.), CuI ([7681-65-4], 1.5 mg, 0.004 mmol, 0.1 eq.), and Et₃N (16 μL, 0.12 mmol, 1.5 eq.) in 1,4-dioxane (0.5 mL) were stirred vigorously at 70° C. for 2 h. Aqueous NH₄Cl (10%) was added and the mixture was extracted with EtOAc. The organic layer was separated, dried (Na₂SO₄), filtered, and the solvent evaporated. The residue was purified by column chromatography (silica, EtOAc in DCM 0/100 to 70/30) to yield Intermediate 232 (32 mg, yield: 70%) as a yellow oil.

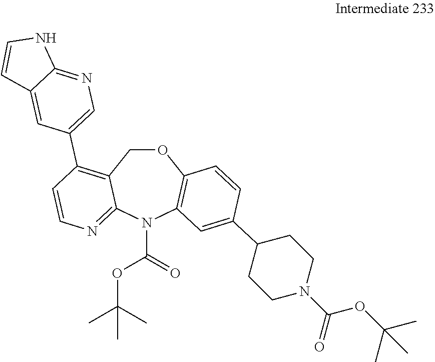 [SMILES: CC(C)(C)OC(=O)N1CCC(c2ccc3c(c2)N(C(=O)OC(C)(C)C)c2nccc(-c4cnc5[nH]ccc5c4)c2CO3)CC1]

Intermediate 233 was synthesized in a similar manner as Intermediate 6 using Intermediate 11 instead of Intermediate 5 and 7-azaindole-5-boronic acid pinacol ester [CAS: 754214-56-7] instead of 3,6-dihydro-2H-pyran-4-boronic acid pinacol ester.

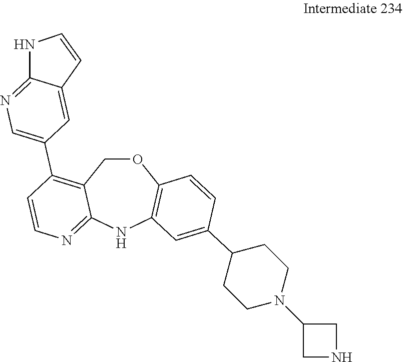 [SMILES: c1cc(-c2cnc3[nH]ccc3c2)c2c(n1)Nc1cc(C3CCN(C4CNC4)CC3)ccc1OC2]

Intermediate 234 was synthesized following the synthetic route from Intermediate 162 to Intermediate 164 starting with Intermediate 233 instead of Intermediate 161.

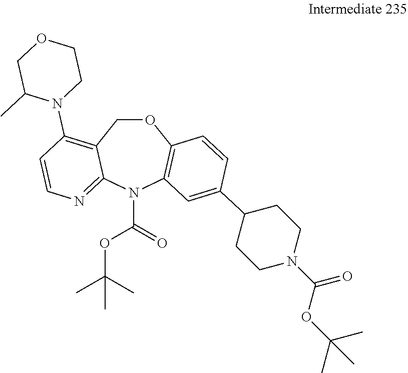 [SMILES: CC1COCCN1c1ccnc2c1COc1ccc(C3CCN(C(=O)OC(C)(C)C)CC3)cc1N2C(=O)OC(C)(C)C]

Intermediate 235 was synthesized in a similar manner as Intermediate 161 using 3-methylmorpholine [CAS: 42185-06-8] instead of thiomorpholine 1,1-dioxide.

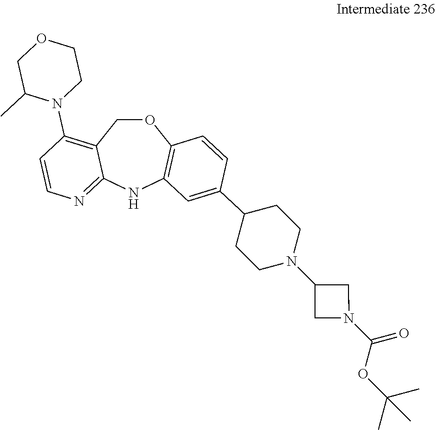 [SMILES: CC1COCCN1c1ccnc2c1COc1ccc(C3CCN(C4CN(C(=O)OC(C)(C)C)C4)CC3)cc1N2]

Intermediate 236 was synthesized following the synthetic route from Intermediate 162 to Intermediate 163 starting with Intermediate 235 instead of Intermediate 161.

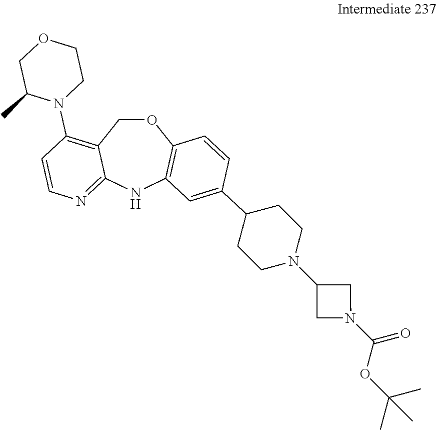 [SMILES: C[C@H]1COCCN1c1ccnc2c1COc1ccc(C3CCN(C4CN(C(=O)OC(C)(C)C)C4)CC3)cc1N2]

(*S), pure stereoisomer but absolute stereochemistry undetermined A batch of Intermediate 236 (250 mg) was separated into its enantiomers using chiral SFC (Stationary phase: Chiralcel OD-H 5 μm 250*30 mm, Mobile phase: 70% CO₂, 30% EtOH (0.3% iPrNH₂)) to afford Intermediate 237 (200 mg) and its enantiomer (48 mg).

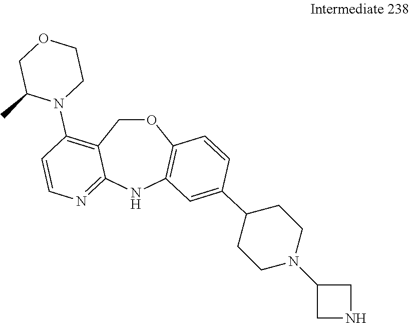 [SMILES: C[C@H]1COCCN1c1ccnc2c1COc1ccc(C3CCN(C4CNC4)CC3)cc1N2]

(*S), pure stereoisomer but absolute stereochemistry undetermined Intermediate 238 was synthesized in a similar manner as Intermediate 164 starting with Intermediate 237 instead of Intermediate 163.

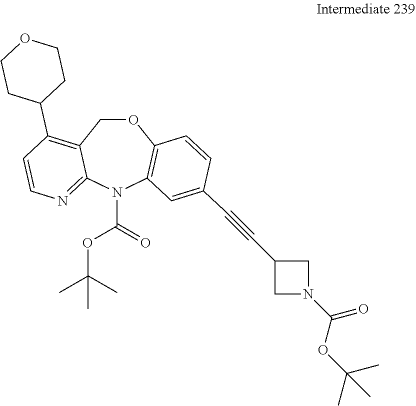 [SMILES: CC(C)(C)OC(=O)N1CC(C#Cc2ccc3c(c2)N(C(=O)OC(C)(C)C)c2nccc(C4CCOCC4)c2CO3)C1]

Intermediate 239 was synthesized in a similar manner as Intermediate 232 starting from tert-butyl 3-ethynylazetidine-1-carboxylate [CAS: 287193-01-5] instead of 1,1-dimethylethyl 4-ethynyl-1-piperidinecarboxylate.

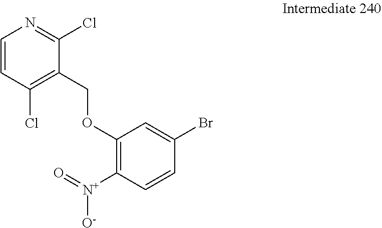 [SMILES: O=[N+]([O-])c1ccc(Br)cc1OCc1c(Cl)ccnc1Cl]

Intermediate 240 was synthesized in a similar manner as Intermediate 1 using 5-bromo-2-nitrophenol [CAS: 27684-84-0] instead of 4-bromo-2-nitrophenol.

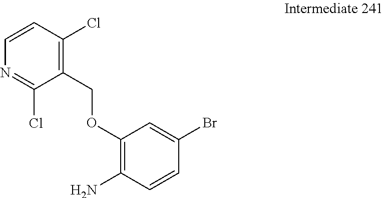 [SMILES: Nc1ccc(Br)cc1OCc1c(Cl)ccnc1Cl]

Intermediate 241 was synthesized in a similar manner as Intermediate 2 using Intermediate 240 instead of Intermediate 1.

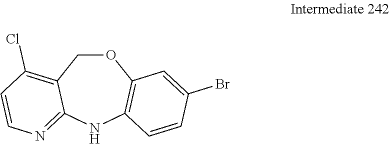 [SMILES: Clc1ccnc2c1COc1cc(Br)ccc1N2]

Intermediate 242 was synthesized in a similar manner as Intermediate 3 using Intermediate 241 instead of Intermediate 2.

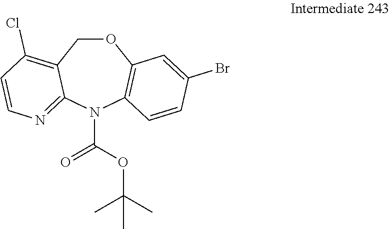 [SMILES: CC(C)(C)OC(=O)N1c2ccc(Br)cc2OCc2c(Cl)ccnc21]

Intermediate 243 was synthesized in a similar manner as Intermediate 19 using Intermediate 242 instead of Intermediate 18.

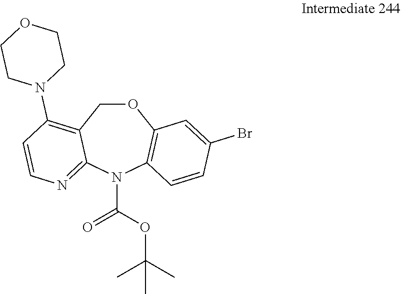 [SMILES: CC(C)(C)OC(=O)N1c2ccc(Br)cc2OCc2c(N3CCOCC3)ccnc21]

Intermediate 244 was synthesized in a similar manner as Intermediate 179 using Intermediate 243 instead of Intermediate 143.

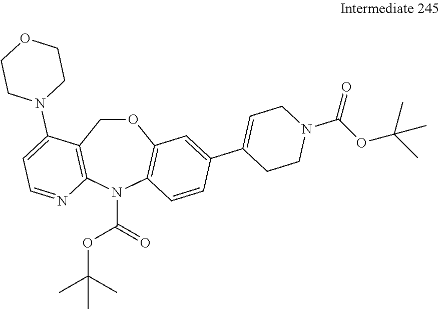 [SMILES: CC(C)(C)OC(=O)N1CC=C(c2ccc3c(c2)OCc2c(N4CCOCC4)ccnc2N3C(=O)OC(C)(C)C)CC1]

Intermediate 245 was synthesized in a similar manner as Intermediate 5 using Intermediate 244 instead of Intermediate 4.

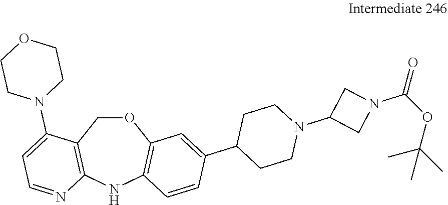 [SMILES: CC(C)(C)OC(=O)N1CC(N2CCC(c3ccc4c(c3)OCc3c(N5CCOCC5)ccnc3N4)CC2)C1]

Intermediate 246 was synthesized following the synthetic route from Intermediate 7 to Intermediate 9 starting with Intermediate 245 instead of Intermediate 6.

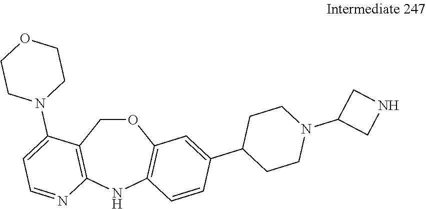 [SMILES: c1cc(N2CCOCC2)c2c(n1)Nc1ccc(C3CCN(C4CNC4)CC3)cc1OC2]

Intermediate 247 was synthesized in a similar manner as Intermediate 146 using Intermediate 246 instead of Intermediate 145.

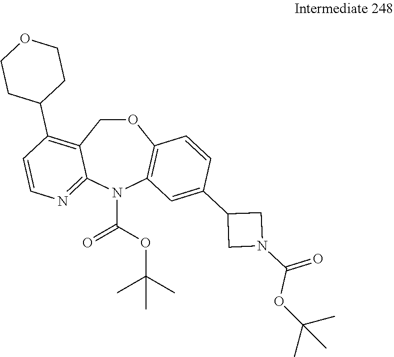 [SMILES: CC(C)(C)OC(=O)N1CC(c2ccc3c(c2)N(C(=O)OC(C)(C)C)c2nccc(C4CCOCC4)c2CO3)C1]

[1-[(1,1-Dimethylethoxy)carbonyl]-3-azetidinyl]iodozine LiCl ([CAS: 2301956-67-0], 0.25 M, 1.3 mL, 0.3 mmol, 2.0 eq.) was added to Intermediate 231 (80 mg, 0.16 mmol) and Pd(OAc)₂ (2 mg, 0.008 mmol, 0.05 eq.). The reaction mixture was stirred at 50° C. for 2 h. The reaction was quenched with 10% aqueous NH₄Cl and 32% aqueous NH₄OH and the reaction mixture was extracted with EtOAc. The combined organic layers were dried (Na₂SO₄), filtered, and the solvent evaporated. The residue was purified by column chromatography (silica, EtOAc in DCM 0/100 to 75/25) to yield Intermediate 248 (77 mg, yield: 91%) as a yellow oil.

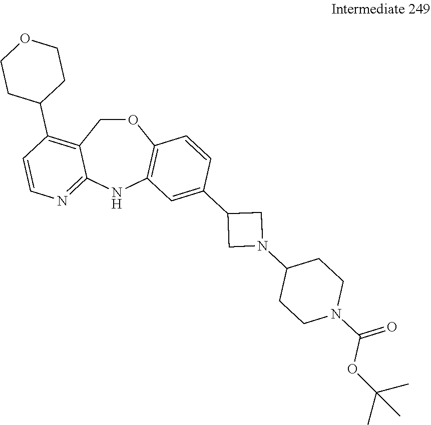 [SMILES: CC(C)(C)OC(=O)N1CCC(N2CC(c3ccc4c(c3)Nc3nccc(C5CCOCC5)c3CO4)C2)CC1]

HCl (4 M in dioxane, 1.0 mL, 4.0 mmol, 28.0 eq.) was added to Intermediate 248 (77 mg, 0.14 mmol) and the mixture was stirred for 1 h at room temperature. The solvent was evaporated. The residue was taken up with DCM and basified with saturated NaHCO₃. The organic layer was separated, dried, (Na₂SO₄), filtered, and the solvent evaporated. The residue was taken up with DCM (1 mL) and tert-butyl 4-oxopiperidine-1-carboxylate ([CAS: 79099-07-3], 43 mg, 0.22 mmol, 1.5 eq.), AcOH (15 μL, 0.26 mmol, 1.8 eq.), and lastly sodium triacetoxyborohydride (61 mg, 0.29 mmol, 2.0 eq.) were added and the mixture was stirred at room temperature overnight. The reaction mixture was basified with saturated NaHCO₃ and extracted with DCM. The organic layer was dried (Na₂SO₄), filtered, and the solvent evaporated. The residue was purified by column chromatography (silica, MeOH in EtOAc 0/100 to 30/70) to yield Intermediate 249 (35 mg, yield: 47%) as a clear oil.

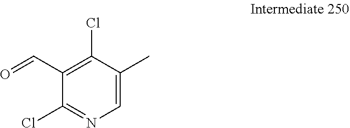

NaBH₄ (1.1 g, 28.9 mmol) was added in small portions to a solution of 2,4-dichloro-5-methyl-3-pyridinecarboxaldehyde ([CAS: 2369720-14-7], 5.5 g, 28.9 mmol) in MeOH (100 mL) at 0° C. The mixture was stirred at room temperature for 2 h. Water (200 mL) was added slowly. The mixture was extracted with EtOAc (200 mL*2). The combined organic layer was washed with brine (200 mL), dried over Na₂SO₄, and evaporated. The residue was purified by flash column chromatography over silica gel (eluent: petroleum ether/EtOAc from 100/0 to 50/50) to give Intermediate 250 (4.3 g, yield: 77%) as a white solid.

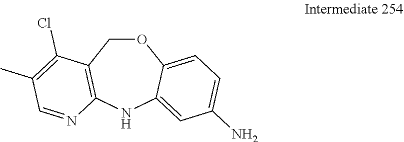 [SMILES: Cc1cnc2c(c1Cl)COc1ccc(N)cc1N2]

TFA (30.2 mL, 394.4 mmol, 30 eq.) was added at 0° C. to a solution of Intermediate 150 (4.8 g, 13.3 mmol) in DCM (64.7 mL). The reaction mixture was stirred at room temperature overnight. The solvent was evaporated. The residue was taken up with DCM and NH₄OH (30% in water). This mixture was extracted twice with DCM. The combined organic layer was dried over MgSO₄, filtered, and concentrated to give Intermediate 254 (3.65 g, quantitative yield).

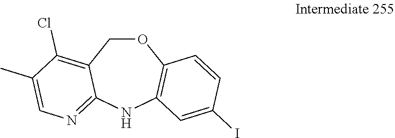 [SMILES: Cc1cnc2c(c1Cl)COc1ccc(I)cc1N2]

Intermediate 255 was synthesized in a similar manner as Intermediate 155 using Intermediate 254 instead of Intermediate 154.

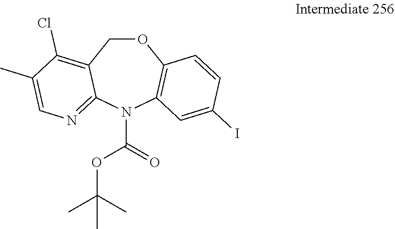 [SMILES: Cc1cnc2c(c1Cl)COc1ccc(I)cc1N2C(=O)OC(C)(C)C]

Intermediate 256 was synthesized in a similar manner as Intermediate 4 using Intermediate 255 instead of Intermediate 3.

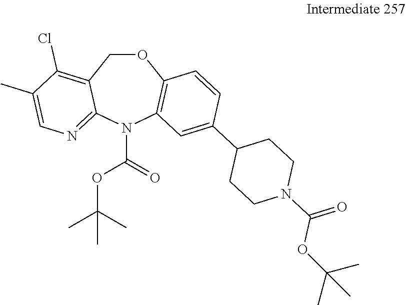 [SMILES: Cc1cnc2c(c1Cl)COc1ccc(C3CCN(C(=O)OC(C)(C)C)CC3)cc1N2C(=O)OC(C)(C)C]

Intermediate 257 was synthesized in a similar manner as Intermediate 11 using Intermediate 256 instead of Intermediate 4.

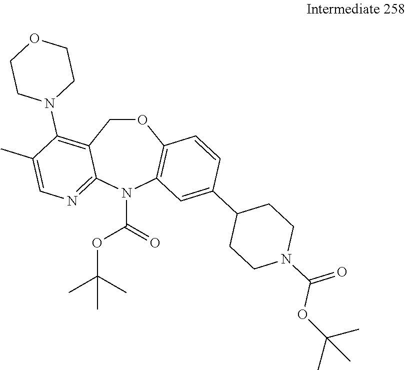 [SMILES: Cc1cnc2c(c1N1CCOCC1)COc1ccc(C3CCN(C(=O)OC(C)(C)C)CC3)cc1N2C(=O)OC(C)(C)C]

Intermediate 258 was synthesized in a similar manner as Intermediate 12 using Intermediate 257 instead of Intermediate 11.

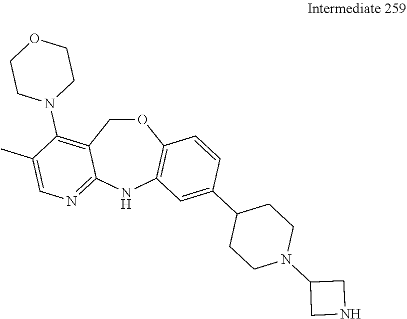 [SMILES: Cc1cnc2c(c1N1CCOCC1)COc1ccc(C3CCN(C4CNC4)CC3)cc1N2]

Intermediate 259 was synthesized following the synthetic route of Intermediate 162 to Intermediate 164 starting with Intermediate 258 instead of Intermediate 161.

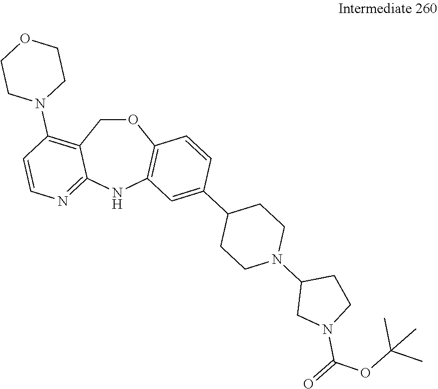 [SMILES: CC(C)(C)OC(=O)N1CCC(N2CCC(c3ccc4c(c3)Nc3nccc(N5CCOCC5)c3CO4)CC2)C1]

Intermediate 260 was synthesized in a similar manner as Intermediate 9 using N-Boc pyrrolidin-3-one [CAS: 101385-93-7] instead of 1-Boc-3-azetidinone.

### Intermediate 260A and Intermediate 260B

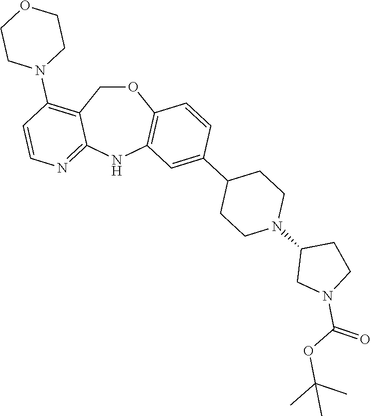

Intermediate 260A: (*R), pure stereoisomer but absolute stereochemistry undetermined

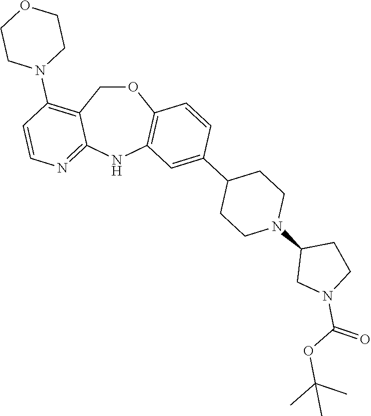

Intermediate 260B: (*S), pure stereoisomer but absolute stereochemistry undetermined The isomers of Intermediate 260 were separated by chiral SFC (Stationary phase: Whelk-O1 (S,S) 5 μm 250*21.2 mm, Mobile phase: 53% CO₂, (47% iPrOH(0.3% iPrNH₂)+20% DCM) to give Intermediate 260A (600 mg, yield 39%) and Intermediate 260B (636 mg, yield 42%).

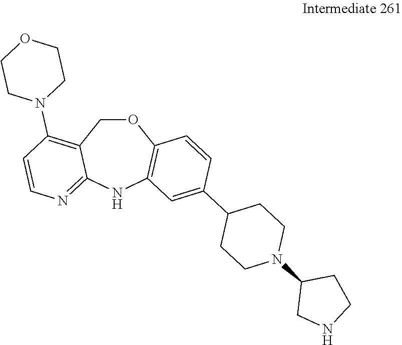 [SMILES: c1cc(N2CCOCC2)c2c(n1)Nc1cc(C3CCN([C@H]4CCNC4)CC3)ccc1OC2]

(*S), pure stereoisomer but absolute stereochemistry undetermined

Intermediate 261 was synthesized in a similar manner as Intermediate 164 using Intermediate 260B instead of Intermediate 163.

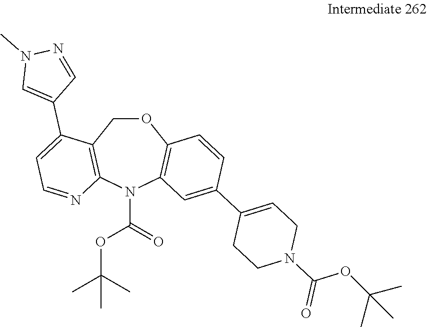 [SMILES: Cn1cc(-c2ccnc3c2COc2ccc(C4=CCN(C(=O)OC(C)(C)C)CC4)cc2N3C(=O)OC(C)(C)C)cn1]

Intermediate 262 was synthesized in a similar manner as Intermediate 6 using 1-methyl-4-(4,4,5,5-tetramethyl-1,3,2-dioxaborolan-2-yl)-1H-pyrazole [CAS: 761446-44-0] instead of 3,6-dihydro-2H-pyran-4-boronic acid pinacol ester.

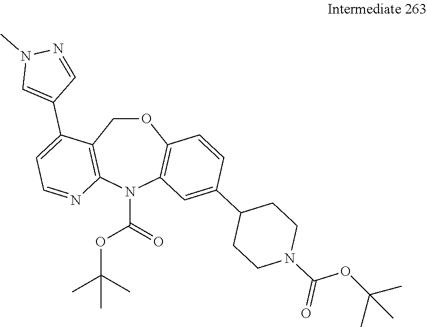 [SMILES: Cn1cc(-c2ccnc3c2COc2ccc(C4CCN(C(=O)OC(C)(C)C)CC4)cc2N3C(=O)OC(C)(C)C)cn1]

Intermediate 263 was synthesized in a similar manner as Intermediate 7 using Intermediate 262 instead of Intermediate 6.

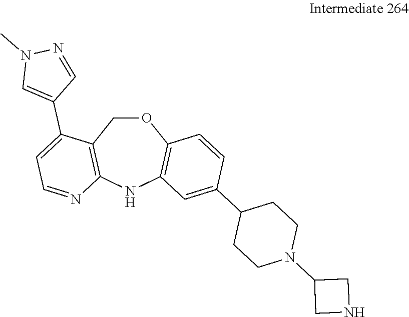 [SMILES: Cn1cc(-c2ccnc3c2COc2ccc(C4CCN(C5CNC5)CC4)cc2N3)cn1]

Intermediate 264 was synthesized following the synthetic route from Intermediate 48 to Intermediate 50 starting with Intermediate 263 instead of Intermediate 47.

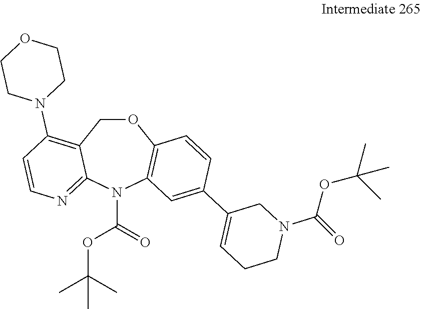 [SMILES: CC(C)(C)OC(=O)N1CCC=C(c2ccc3c(c2)N(C(=O)OC(C)(C)C)c2nccc(N4CCOCC4)c2CO3)C1]

Intermediate 265 was synthesized in a similar manner as Intermediate 20 using Intermediate 175 instead of Intermediate 19 and 1-Boc-5,6-dihydro-2H-pyridine-3-boronic acid pinacol ester [CAS: 885693-20-9] instead of N-Boc-1,2,3,6-tetrahydropyridine-4-boronic acid pinacol ester.

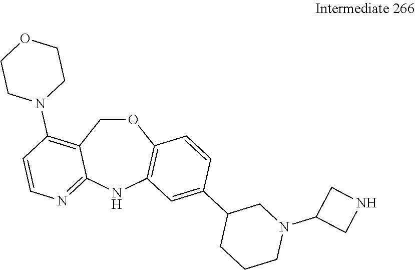 [SMILES: c1cc(N2CCOCC2)c2c(n1)Nc1cc(C3CCCN(C4CNC4)C3)ccc1OC2]

Intermediate 266 was synthesized following the synthetic route from Intermediate 22 to Intermediate 25 starting with Intermediate 265 instead of Intermediate 21.

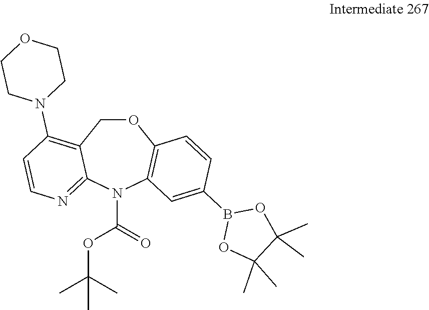 [SMILES: CC(C)(C)OC(=O)N1c2cc(B3OC(C)(C)C(C)(C)O3)ccc2OCc2c(N3CCOCC3)ccnc21]

Pd(dppf)Cl₂ (115 mg, 0.14 mmol, 0.05 eq.) was added to a solution of Intermediate 175 (1.3 g, 2.8 mmol), bis(pinacolate)diboron ([CAS: 73183-34-3], 928 mg, 3.7 mmol, 1.3 eq.), and KOAc (414 mg, 4.2 mmol, 1.5 eq.) in 1,4-dioxane (22.5 mL) while the reaction was degassed by bubbling nitrogen through the solution. The reaction mixture was heated at 80° C. in a sealed tube for 4 h. The reaction mixture was diluted with EtOAc, washed with water and brine, dried over MgSO₄, filtered, and concentrated to dryness. The residue was purified by flash column chromatography (SiO₂, hexane/EtOAc gradient) to afford Intermediate 267 (1.5 g, yield: 91%).

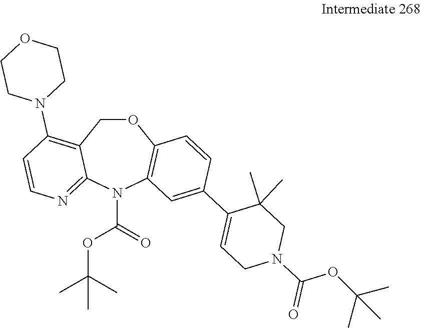 [SMILES: CC(C)(C)OC(=O)N1CC=C(c2ccc3c(c2)N(C(=O)OC(C)(C)C)c2nccc(N4CCOCC4)c2CO3)C(C)(C)C1]

Intermediate 268 was synthesized in a similar manner as Intermediate 20 using Intermediate 267 instead of Intermediate 19 and tert-butyl 3,3-dimethyl-4-(((trifluoromethyl)sulfonyl)oxy)-3,6-dihydropyridine-1(2H)-carboxylate [CAS: 324769-08-6] instead of N-Boc-1,2,3,6-tetrahydropyridine-4-boronic acid pinacol ester.

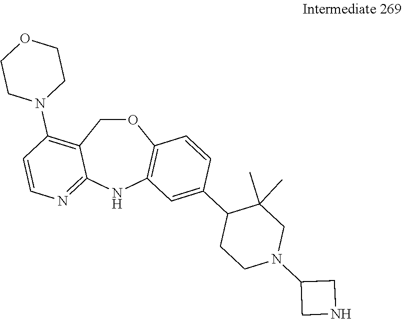 [SMILES: CC1(C)CN(C2CNC2)CCC1c1ccc2c(c1)Nc1nccc(N3CCOCC3)c1CO2]

Intermediate 269 was synthesized following the synthetic route from Intermediate 22 to Intermediate 25 starting with Intermediate 268 instead of Intermediate 21.

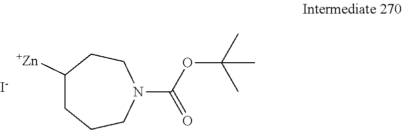

1,2-dibromoethane ([CAS: 106-93-4], 42 μL, 0.49 mmol, 0.09 eq.) was added to a suspension of Zn (427 mg, 6.5 mmol, 1.2 eq.) in DMA (7.5 mL) under nitrogen atmosphere. The mixture was heated briefly with a heat gun and allowed to cool to room temperature (3 times). TMS-Cl (41 μL, 0.33 mmol, 0.06 eq.) was added slowly and the mixture was stirred at room temperature under nitrogen atmosphere for 30 min. 1,1-Dimethylethyl hexahydro-4-iodo-1H-azepine-1-carboxylate ([CAS: 1394839-99-6], 1.77 g, 5.4 mmol) in DMA (7.5 mL) was added dropwise at such a rate that the temperature did not exceed 50° C. (15 min) and the reaction mixture was stirred for 0.5 h. The solution of Intermediate 270 (2.13 g, quantitative yield) was used without further purification in the next step.

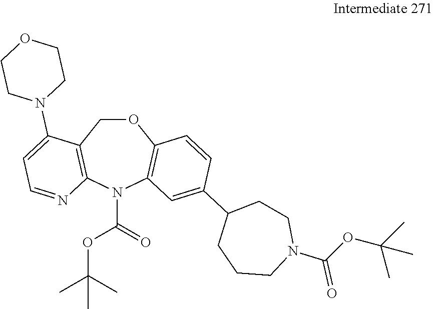 [SMILES: CC(C)(C)OC(=O)N1CCCC(c2ccc3c(c2)N(C(=O)OC(C)(C)C)c2nccc(N4CCOCC4)c2CO3)CC1]

Intermediate 271 was synthesized in a similar manner as Intermediate 78 using Intermediate 175 instead of 2-chloro-5-(methoxymethoxy)pyridine, and Intermediate 270 instead of [1-(tert-butoxycarbonyl)piperidin-4-yl]zinc iodide.

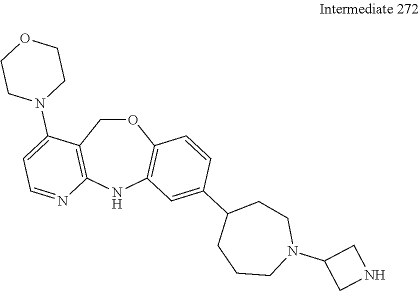 [SMILES: c1cc(N2CCOCC2)c2c(n1)Nc1cc(C3CCCN(C4CNC4)CC3)ccc1OC2]

Intermediate 272 was synthesized following the synthetic route of Intermediate 22 to Intermediate 24 starting with Intermediate 271 instead of Intermediate 21.

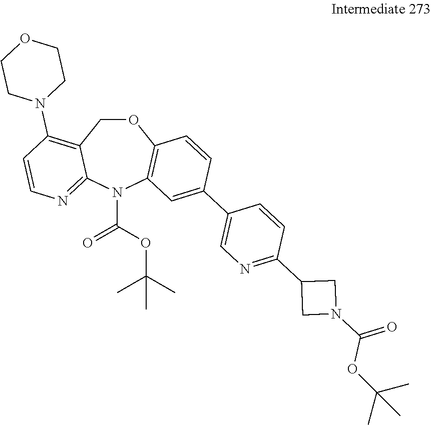 [SMILES: CC(C)(C)OC(=O)N1CC(c2ccc(-c3ccc4c(c3)N(C(=O)OC(C)(C)C)c3nccc(N5CCOCC5)c3CO4)cn2)C1]

Intermediate 273 was synthesized in a similar manner as Intermediate 211 using Intermediate 215 instead of Intermediate 210 and [1-[(1,1-dimethylethoxy)carbonyl]-3-azetidinyl]iodozine [CAS: 206446-38-0] instead of [1-[(1,1-dimethylethoxy)carbonyl]-4-piperidinyl]iodozine.

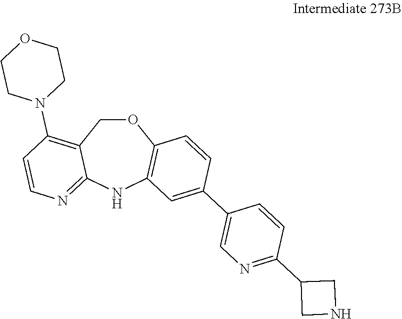 [SMILES: c1cc(N2CCOCC2)c2c(n1)Nc1cc(-c3ccc(C4CNC4)nc3)ccc1OC2]

Intermediate 273B was synthesized in a similar manner as Intermediate 222 using Intermediate 273 instead of Intermediate 221.

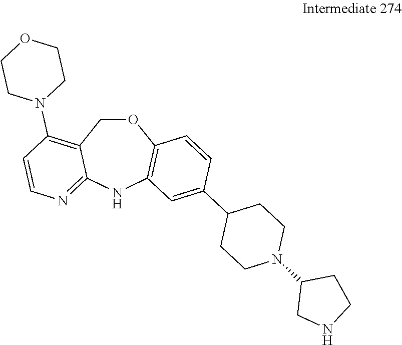 [SMILES: c1cc(N2CCOCC2)c2c(n1)Nc1cc(C3CCN([C@@H]4CCNC4)CC3)ccc1OC2]

(*R), pure stereoisomer but absolute stereochemistry undetermined Intermediate 274 was synthesized in a similar manner as Intermediate 164 using Intermediate 260A instead of Intermediate 163.

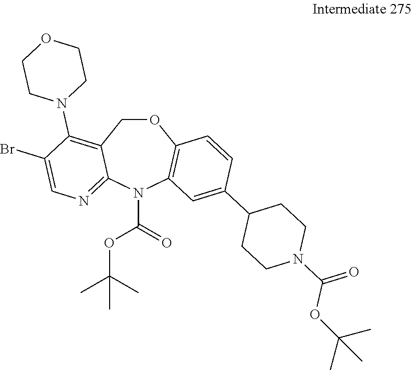 [SMILES: CC(C)(C)OC(=O)N1CCC(c2ccc3c(c2)N(C(=O)OC(C)(C)C)c2ncc(Br)c(N4CCOCC4)c2CO3)CC1]

Intermediate 12 (700 mg, 1.23 mmol) and N-bromosuccinimide ([CAS: 128-08-5], 549 mg, 3.1 mmol) in DMF(12 mL) were stirred at room temperature for 4 h. Water and EtOAc were added and the reaction mixture was extracted. The organic layer was dried over MgSO₄, filtered, and evaporated. The residue was purified by column chromatography (Stationary phase: irregular SiOH 15-40 μm 4 g, gradient from 80/20 to 60/40 heptane/EtOAc) to give Intermediate 275 (640 mg, yield: 80%).

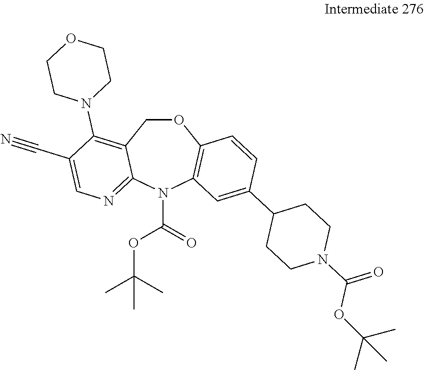 [SMILES: CC(C)(C)OC(=O)N1CCC(c2ccc3c(c2)N(C(=O)OC(C)(C)C)c2ncc(C#N)c(N4CCOCC4)c2CO3)CC1]

A mixture of Intermediate 275 (800 mg, 1.24 mmol), zinc cyanide ([CAS: 557-21-1], 145 mg, 1.24 mmol, 1.0 eq.), zinc dust ([CAS: 7440-66-6], 40 mg, 0.62 mmol), Pd₂dba₃ (57 mg, 0.062 mmol), and 1,1′-bis(diphenylphosphino)ferrocene ([CAS: 12150-46-8], 86 mg, 0.15 mmol) in DMA (10 mL) was stirred at 90° C. for 1 h under microwave irradiation. Water and EtOAc were added and the reaction mixture was extracted. The organic layer was dried over MgSO₄, filtered, and evaporated. The residue was purified by column chromatography (Stationary phase: irregular SiOH 15-40 μm 25 g, gradient from 80/20 to 60/40 heptane/EtOAc), followed by another column chromatography (Stationary phase: irregular SiOH 15-40 μm 12 g, gradient from 80/20 to 60/40 heptane/EtOAc) to yield Intermediate 276 (490 mg, yield: 67%).

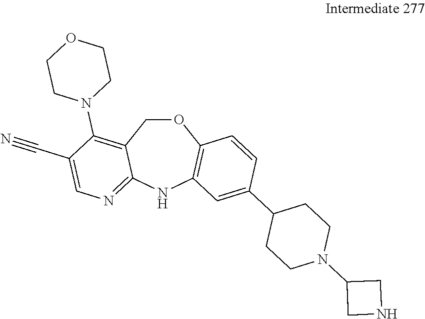 [SMILES: N#Cc1cnc2c(c1N1CCOCC1)COc1ccc(C3CCN(C4CNC4)CC3)cc1N2]

Intermediate 277 was synthesized following the synthetic route of Intermediate 162 to Intermediate 164 starting with Intermediate 276 instead of Intermediate 161.

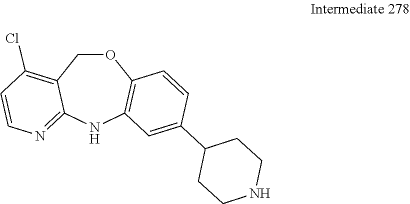 [SMILES: Clc1ccnc2c1COc1ccc(C3CCNCC3)cc1N2]

Intermediate 278 was synthesized in a similar manner as Intermediate 162 using Intermediate 11 instead of Intermediate 161.

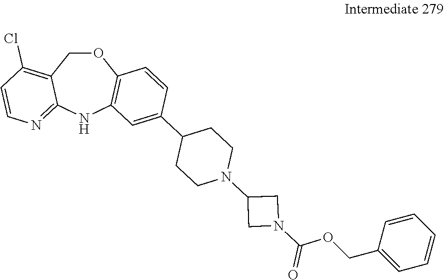 [SMILES: O=C(OCc1ccccc1)N1CC(N2CCC(c3ccc4c(c3)Nc3nccc(Cl)c3CO4)CC2)C1]

Intermediate 279 was synthesized in a similar manner as Intermediate 163 using Intermediate 278 instead of Intermediate 162 and benzyl 3-oxoazetidine-1-carboxylate [CAS: 105258-93-3] instead of 1-Boc-3-azetidinone.

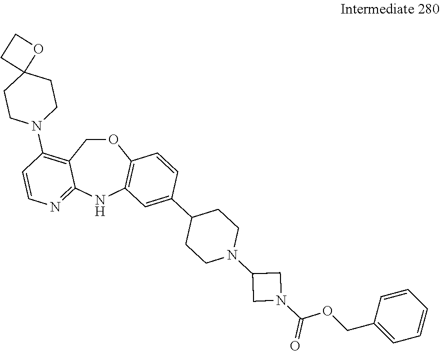 [SMILES: O=C(OCc1ccccc1)N1CC(N2CCC(c3ccc4c(c3)Nc3nccc(N5CCC6(CCO6)CC5)c3CO4)CC2)C1]

Intermediate 280 was synthesized in a similar manner as Intermediate 169 using Intermediate 279 instead of Intermediate 11 and 1-oxa-7-azaspiro[3.5]nonane [CAS: 38674-21-4] instead of 8-oxa-3-azabicyclo[3.2.1]octane.

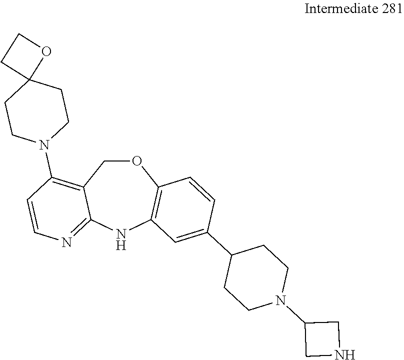 [SMILES: c1cc(N2CCC3(CCO3)CC2)c2c(n1)Nc1cc(C3CCN(C4CNC4)CC3)ccc1OC2]

Intermediate 281 was synthesized in a similar manner as Intermediate 8 using Intermediate 280 instead of Intermediate 7.

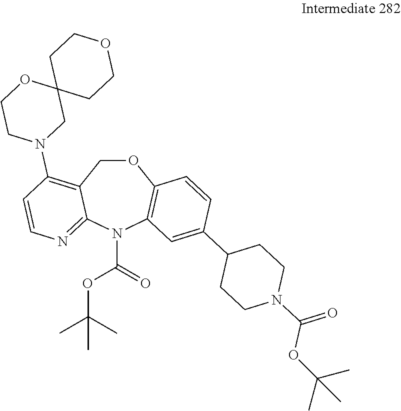 [SMILES: CC(C)(C)OC(=O)N1CCC(c2ccc3c(c2)N(C(=O)OC(C)(C)C)c2nccc(N4CCOC5(CCOCC5)C4)c2CO3)CC1]

Intermediate 282 was synthesized in a similar manner as Intermediate 169 using 1,9-dioxa-4-azaspiro[5,5]undecane [CAS: 402938-74-3] instead of 8-oxa-3-azabicyclo[3.2.1]octane.

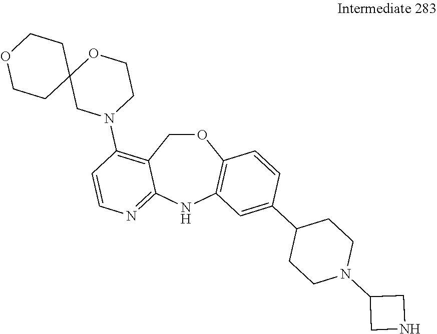 [SMILES: c1cc(N2CCOC3(CCOCC3)C2)c2c(n1)Nc1cc(C3CCN(C4CNC4)CC3)ccc1OC2]

Intermediate 283 was synthesized following the synthetic route of Intermediate 162 to Intermediate 164 starting with Intermediate 282 instead of Intermediate 161.

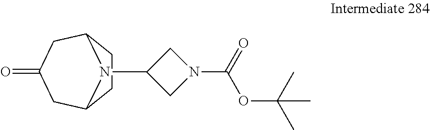 [SMILES: CC(C)(C)OC(=O)N1CC(N2C3CCC2CC(=O)C3)C1]

A solution of 8,8-dimethyl-3-oxo-8-azoniabicyclo[3.2.1]octane ([CAS: 223741-88-6], 4.9 g, 17.4 mmol, 1.0 eq.) and 3-amino-1-N-Boc-azetidine [CAS: 193269-78-2], 3.0 g, 17.4 mmol, 1.0 eq.) in a mixture of EtOH (39 mL) and water (39 mL) was heated to reflux temperature. Potassium carbonate (7.2 g, 52.3 mmol, 3.0 eq.) was added portionwise over 15 min and the reaction mixture was then refluxed for additional 14 h. The reaction mixture was cooled to room temperature and extracted with DCM. The organic layer was washed with brine, then dried with MgSO₄, and concentrated. The residue was purified by column flash chromatography (SiO₂, EtOAc/heptane gradient) to give Intermediate 284 (4.9 g, yield: 58%).

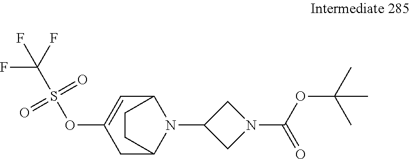 [SMILES: CC(C)(C)OC(=O)N1CC(N2C3C=C(OS(=O)(=O)C(F)(F)F)CC2CC3)C1]

Lithium bis(trimethylsilyl)-amide (1 M in THF, 17.2 mL, 17.2 mmol, 1.7 eq.) was added to a solution of Intermediate 284 (2.8 g, 10.1 mmol, 1.0 eq.) in dry THF under nitrogen at −60° C. and the mixture was stirred at −60° C. for 15 min. A solution of N-phenyl-bis(trifluoro-methanesultonimide) ([CAS: 37595-74-7], 4.7 g, 13.2 mmol, 1.3 eq.) in THF (35 mL) was added and the mixture was stirred for 30 min at −60° C. The reaction was allowed to warm to room temperature and was stirred at room temperature for 2 h. The mixture was poured into saturated aqueous NaHCO₃ and extracted with EtOAc. The organic layer was washed with brine, dried with MgSO₄, and concentrated. The residue was purified by flash column chromatography (SiO₂, hexane/EtOAc gradient) to afford Intermediate 285 (3.4 g, yield: 82%).

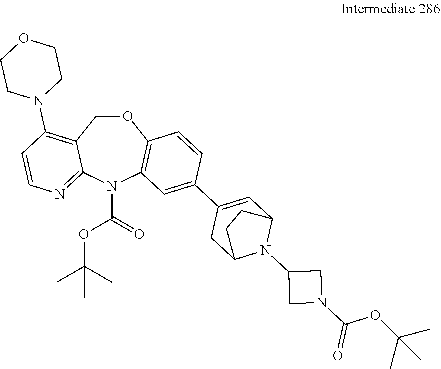 [SMILES: CC(C)(C)OC(=O)N1CC(N2C3C=C(c4ccc5c(c4)N(C(=O)OC(C)(C)C)c4nccc(N6CCOCC6)c4CO5)CC2CC3)C1]

Intermediate 286 was synthesized in a similar manner as Intermediate 20 using Intermediate 267 instead of Intermediate 19 and Intermediate 285 instead of N-Boc-1,2,3,6-tetrahydropyridine-4-boronic acid pinacol ester.

### Intermediate 287A and Intermediate 287B

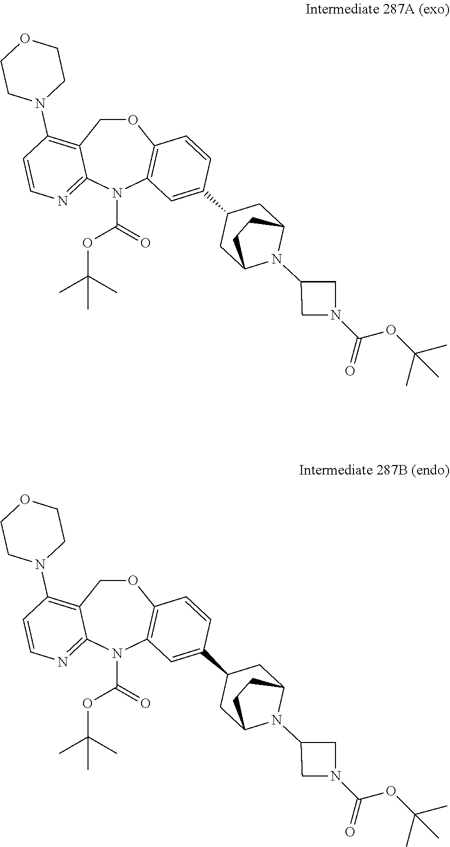 [SMILES: CC(C)(C)OC(=O)N1CC(N2[C@@H]3CC[C@H]2C[C@H](c2ccc4c(c2)N(C(=O)OC(C)(C)C)c2nccc(N5CCOCC5)c2CO4)C3)C1 ; CC(C)(C)OC(=O)N1CC(N2[C@@H]3CC[C@H]2C[C@@H](c2ccc4c(c2)N(C(=O)OC(C)(C)C)c2nccc(N5CCOCC5)c2CO4)C3)C1]

Pd/C 10% (wet, 47 mg) was added to a solution of Intermediate 286 (678 mg, 0.94 mmol) in MeOH (19 mL) under nitrogen atmosphere. The reaction mixture was purged first with nitrogen and then with hydrogen. The reaction mixture was stirred under hydrogen atmosphere for 15 h at room temperature. Acetic acid (1.5 mL) was added. The mixture was purged with nitrogen, then with hydrogen, and stirred overnight at room temperature. Acetic acid (1.5 mL) and Pd/C 10% (wet, 47 mg) were added again and the mixture was purged with nitrogen, then with hydrogen, and stirred overnight. The reaction mixture was filtered through a short path of Celite and the cake was washed with MeOH and DCM. The combined filtrates were concentrated and the residue was purified by reverse phase chromatography (Phenomenex Gemini C18 30×100 mm 5 μm; gradient from 72% [65 mM NH₄OAc+ACN (90:10)]−28% ACN to 36% [65 mM NH₄OAc+ACN (90:10)]−64% ACN) to give Intermediate 287A (168 mg, yield: 27%) and Intermediate 287B (242 mg, yield: 40%).

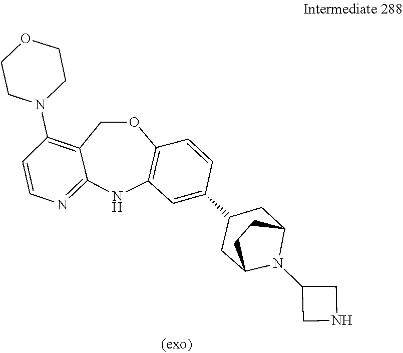

Intermediate 288 was synthesized in a similar manner as Intermediate 23 using Intermediate 287A instead of Intermediate 22.

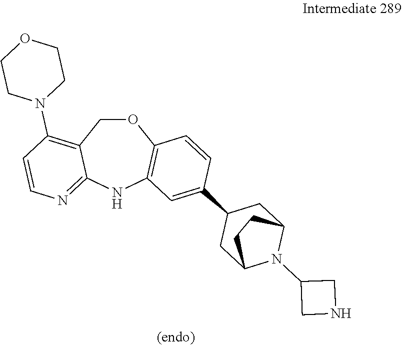 [SMILES: c1cc(N2CCOCC2)c2c(n1)Nc1cc([C@H]3C[C@H]4CC[C@@H](C3)N4C3CNC3)ccc1OC2]

Intermediate 289 was synthesized in a similar manner as Intermediate 23 using Intermediate 287B instead of Intermediate 22.

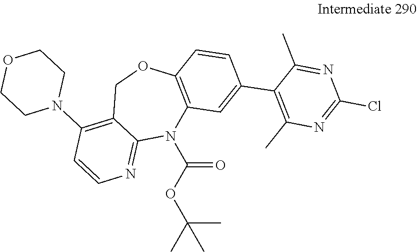 [SMILES: Cc1nc(Cl)nc(C)c1-c1ccc2c(c1)N(C(=O)OC(C)(C)C)c1nccc(N3CCOCC3)c1CO2]

Intermediate 267 (1.4 g, 2.8 mmol, 1.0 eq.), 5-bromo-2-chloro-4,6-dimethylpyrimidine ([CAS: 4786-72-5], 918 mg, 4.1 mmol, 1.5 eq.), PdCl₂(PPh₃)₂ ([CAS: 13965-03-2], 194 mg, 0.28 mmol, 0.1 eq.), and Na₂CO₃ (1 M, 5.5 mL, 5.5 mmol, 2.0 eq.) were suspended in 1,4-dioxane. The mixture was degassed by bubbling nitrogen for 15 min and then heated at 100° C. overnight. The reaction mixture was allowed to cool to room temperature. Brine (15 mL) and EtOAc (70 mL) were added. The organic layer was separated, dried over MgSO₄, filtered, and concentrated. The residue was purified by column chromatography over silica gel (gradient of EtOAc in heptane from 0 to 100%) to afford Intermediate 290 (532 mg, yield: 35%) as a yellowish solid.

### Mixture of Intermediate 291A and Intermediate 2911R

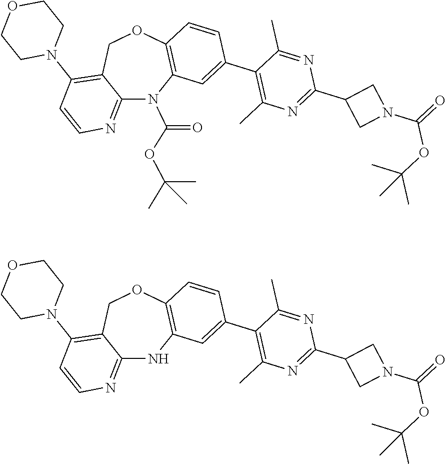 [SMILES: Cc1nc(C2CN(C(=O)OC(C)(C)C)C2)nc(C)c1-c1ccc2c(c1)N(C(=O)OC(C)(C)C)c1nccc(N3CCOCC3)c1CO2 ; Cc1nc(C2CN(C(=O)OC(C)(C)C)C2)nc(C)c1-c1ccc2c(c1)Nc1nccc(N3CCOCC3)c1CO2]

The mixture of Intermediate 291A and Intermediate 291B was synthesized in a similar manner as Intermediate 78 using Intermediate 290 instead of 2-chloro-5-(methoxymethoxy)pyridine and [1-[(1,1-dimethylethoxy)carbonyl]-3-azetidinyl]iodozine [CAS: 206446-38-0] instead of [1-(tert-butoxycarbonyl)piperidin-4-yl]zinc iodide.

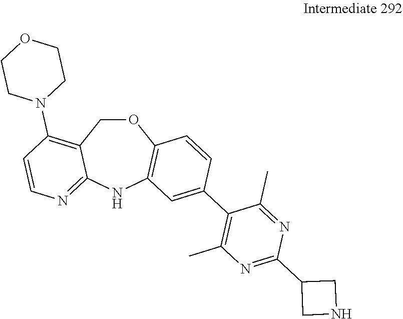 [SMILES: Cc1nc(C2CNC2)nc(C)c1-c1ccc2c(c1)Nc1nccc(N3CCOCC3)c1CO2]

Intermediate 292 was synthesized in a similar manner as Intermediate 114 using the mixture of Intermediate 291A and Intermediate 291B instead of Intermediate 113.

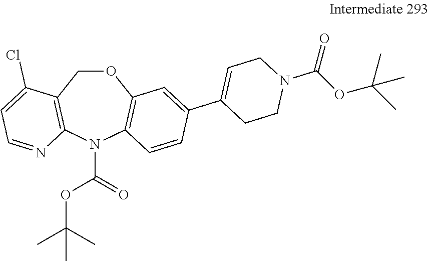 [SMILES: CC(C)(C)OC(=O)N1CC=C(c2ccc3c(c2)OCc2c(Cl)ccnc2N3C(=O)OC(C)(C)C)CC1]

Intermediate 293 was synthesized in a similar manner as Intermediate 5 using Intermediate 243 instead of Intermediate 4.

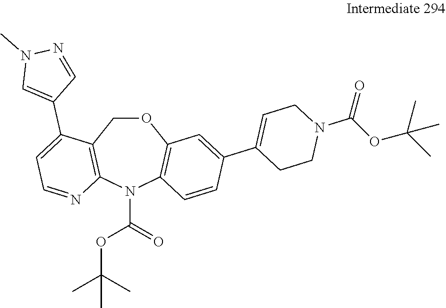 [SMILES: Cn1cc(-c2ccnc3c2COc2cc(C4=CCN(C(=O)OC(C)(C)C)CC4)ccc2N3C(=O)OC(C)(C)C)cn1]

Intermediate 294 was synthesized in a similar manner as Intermediate 6 using Intermediate 293 instead of Intermediate 5 and 1-methylpyrazole-4-boronic acid pinacol ester [CAS: 761446-44-0] instead of 3,6-dihydro-2H-pyran-4-boronic acid pinacol ester.

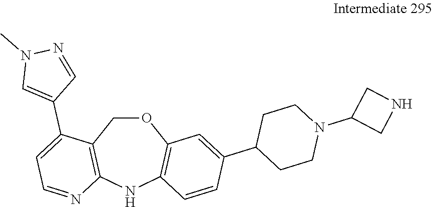 [SMILES: Cn1cc(-c2ccnc3c2COc2cc(C4CCN(C5CNC5)CC4)ccc2N3)cn1]

Intermediate 295 was synthesized following the synthetic route from Intermediate 7 to Intermediate 10 starting with Intermediate 294 instead of Intermediate 6.

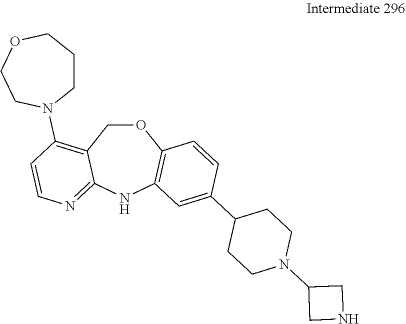 [SMILES: c1cc(N2CCCOCC2)c2c(n1)Nc1cc(C3CCN(C4CNC4)CC3)ccc1OC2]

Intermediate 296 was synthesized following the synthetic route from Intermediate 12 to Intermediate 15 starting with homomorpholine (HCl salt, [CAS: 178312-62-4]) instead of morpholine.

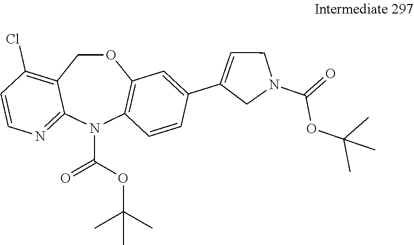 [SMILES: CC(C)(C)OC(=O)N1CC=C(c2ccc3c(c2)OCc2c(Cl)ccnc2N3C(=O)OC(C)(C)C)C1]

Intermediate 297 was synthesized in a similar manner as Intermediate 20 using Intermediate 243 instead of Intermediate 19 and tert-butyl 3-(4,4,5,5-tetramethyl-1,3,2-dioxaborolan-2-yl)-2,5-dihydro-TH-pyrrole-1-carboxylate [CAS: 212127-83-8] instead of N-Boc-1,2,3,6-tetrahydropyridine-4-boronic acid pinacol ester.

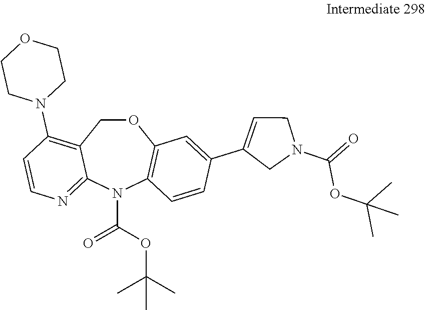 [SMILES: CC(C)(C)OC(=O)N1CC=C(c2ccc3c(c2)OCc2c(N4CCOCC4)ccnc2N3C(=O)OC(C)(C)C)C1]

Intermediate 298 was synthesized in a similar manner as Intermediate 169 using morpholine instead of 8-oxa-3-azabicyclo[3.2.1]octane and Intermediate 297 instead of Intermediate 11.

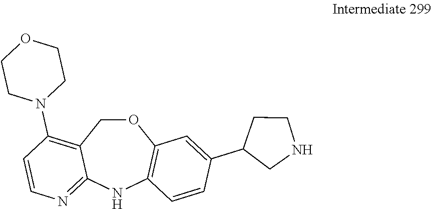 [SMILES: c1cc(N2CCOCC2)c2c(n1)Nc1ccc(C3CCNC3)cc1OC2]

Intermediate 299 was synthesized following the synthetic route from Intermediate 22 to Intermediate 23 starting with Intermediate 298 instead of Intermediate 21.

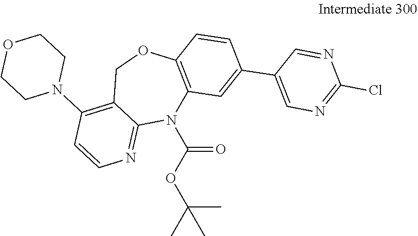 [SMILES: CC(C)(C)OC(=O)N1c2cc(-c3cnc(Cl)nc3)ccc2OCc2c(N3CCOCC3)ccnc21]

Intermediate 300 was synthesized in a similar manner as Intermediate 290 using 5-bromo-2-chloropyrimidine [CAS: 32779-36-5] instead of 5-bromo-2-chloro-4,6-dimethylpyrimidine.

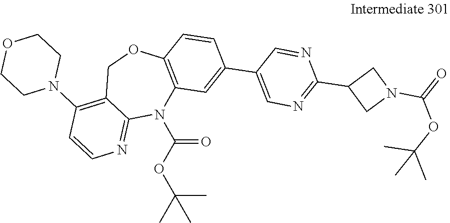 [SMILES: CC(C)(C)OC(=O)N1CC(c2ncc(-c3ccc4c(c3)N(C(=O)OC(C)(C)C)c3nccc(N5CCOCC5)c3CO4)cn2)C1]

Intermediate 300 (285 mg, 0.58 mmol, 1.0 eq.), Pd(dppf)Cl₂ DCM (23 mg, 0.03 mmol, 0.05 eq.), and CuI (11 mg, 0.06 mmol, 0.1 eq.) were placed in solution in DMA (5 mL) under nitrogen atmosphere. A solution of [1-[(1,1-dimethylethoxy)carbonyl]-3-azetidinyl]iodozine ([CAS: 206446-38-0], equivalent to 504 mg, 1.4 mmol, 2.5 eq.) was added via syringe and the resulting mixture was stirred at 80° C. for 1 h under nitrogen atmosphere. The mixture was allowed to cool to room temperature and was diluted with EtOAc (150 mL), saturated aqueous NH₄Cl (50 mL) and water (100 mL). The organic layer was washed with brine (50 mL), dried over MgSO₄, filtered, and concentrated. The residue was purified by flash column chromatography over silica gel (gradient of EtOAc in heptane from 0 to 100%) to give Intermediate 301 (343 mg, yield: 93%) as a brownish oily residue.

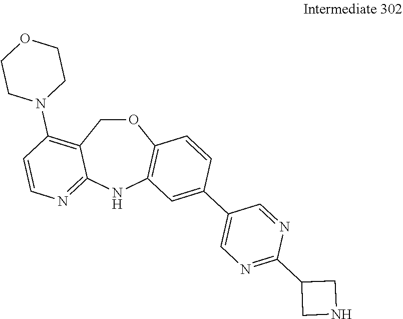 [SMILES: c1cc(N2CCOCC2)c2c(n1)Nc1cc(-c3cnc(C4CNC4)nc3)ccc1OC2]

Intermediate 302 was synthesized in a similar manner as Intermediate 124 using Intermediate 301 instead of Intermediate 123.

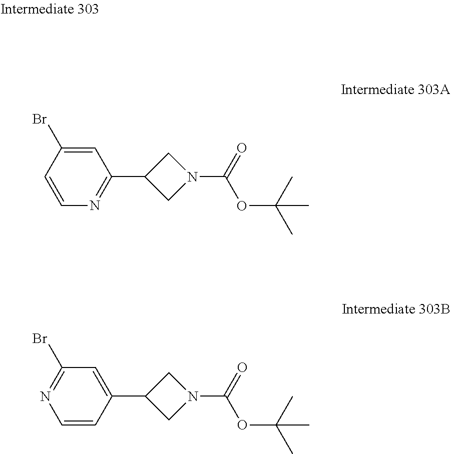 [SMILES: CC(C)(C)OC(=O)N1CC(c2cc(Br)ccn2)C1 ; CC(C)(C)OC(=O)N1CC(c2ccnc(Br)c2)C1]

Intermediate 303A and 303B were synthesized in the same reaction, and were isolated separately, in a similar manner as Intermediate 211 using 2,4-dibromopyridine [CAS: 58530-53-3] instead of Intermediate 210 and [1-[(1,1-dimethylethoxy)carbonyl]-3-azetidinyl]iodozine [CAS: 206446-38-0] instead of [1-[(1,1-dimethylethoxy)carbonyl]-4-piperidinyl]iodozine.

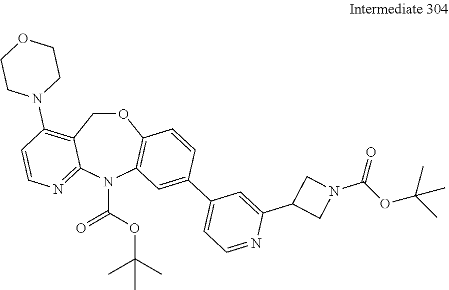 [SMILES: CC(C)(C)OC(=O)N1CC(c2cc(-c3ccc4c(c3)N(C(=O)OC(C)(C)C)c3nccc(N5CCOCC5)c3CO4)ccn2)C1]

Intermediate 304 was synthesized in a similar manner as Intermediate 20 using Intermediate 303A instead of Intermediate 19 and Intermediate 267 instead of N-Boc-1,2,3,6-tetrahydropyridine-4-boronic acid pinacol ester.

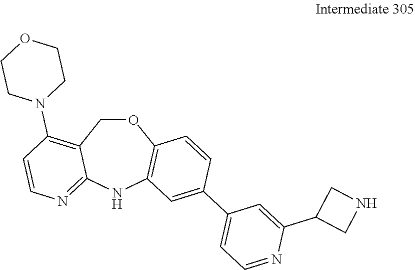 [SMILES: c1cc(-c2ccc3c(c2)Nc2nccc(N4CCOCC4)c2CO3)cc(C2CNC2)n1]

Intermediate 305 was synthesized in a similar manner as Intermediate 124 using Intermediate 304 instead of Intermediate 123.

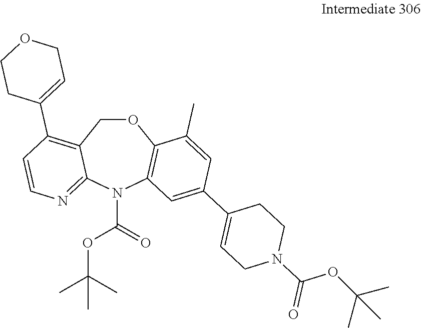 [SMILES: Cc1cc(C2=CCN(C(=O)OC(C)(C)C)CC2)cc2c1OCc1c(C3=CCOCC3)ccnc1N2C(=O)OC(C)(C)C]

In a sealed tube, a solution of Intermediate 20 (1 g, 1.884 mmol), dihydropyran-4-boronic acid pinacol ester (CAS [287944-16-5], 396 mg, 1.884 mmol, 1 eq.), and potassium phosphate (800 mg, 3.768 mmol, 2 eq.) in 1,4-dioxane (13 mL) and water (2 mL) was degassed with nitrogen. Dichloro[1,1′-bis(diphenylphosphino)ferrocene]palladium(II), complex with dichloromethane (1:1) (CAS [95464-05-4], 154 mg, 0.188 mmol, 0.1 eq.) was added. The reaction mixture was degassed again with nitrogen and was stirred at 100° C. for 14 h. The reaction mixture was partitioned between EtOAc and brine. The organic layer was dried (MgSO₄), filtered, and concentrated. The residue was purified by flash column chromatography (silica; heptane/EtOAc gradient) to afford Intermediate 306 (960 mg, yield: 88%).

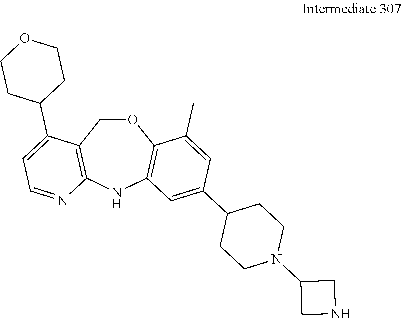 [SMILES: Cc1cc(C2CCN(C3CNC3)CC2)cc2c1OCc1c(C3CCOCC3)ccnc1N2]

Intermediate 307 was synthesized following the synthetic route from Intermediate 22 to Intermediate 25 starting with Intermediate 306 instead of Intermediate 21.

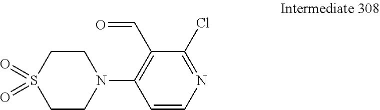 [SMILES: O=Cc1c(N2CCS(=O)(=O)CC2)ccnc1Cl]

2,4-Dichloro-3-pyridinecarboxaldehyde [CAS: 134031-24-6] (1.76 g, 9.99 mmol) and thiomorpholine 1,1-dioxide (2.7 g, 19.97 mmol) were mixed in THF (27 mL) in a Schlenk tube and stirred at 120° C. for 20 min. The precipitate was filtered off. The filtrate was diluted with water and DCM. The organic layer was separated, dried over MgSO₄, filtered, and concentrated. The residue was purified by column chromatography (Stationary phase: irregular SiOH 15-40 μm 120 g, gradient from 80% heptane, 20% EtOAc to 30% heptane, 60% EtOAc, 10% MeOH with 2% NH₄OH) to give Intermediate 308 (6 g, yield 46%).

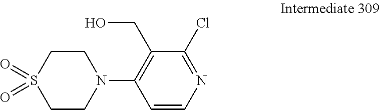 [SMILES: O=S1(=O)CCN(c2ccnc(Cl)c2CO)CC1]

Sodium borohydride [CAS: 16940-66-2] (0.91 g, 24.0 mmol) was added portionwise to an ice cold solution of Intermediate 308 (6 g, 21.8 mmol) in MeOH (150 mL). Upon complete addition, the mixture was allowed to come to room temperature. The reaction mixture was stirred for 3 h. The reaction was quenched with acetone (10 mL). EtOAc (300 mL) was added, followed by saturated aqueous NaHCO₃ (150 mL). The organic layer was separated, and the aqueous phase was extracted once more with EtOAc (200 mL). The combined organic layers were dried over MgSO₄, filtered, and concentrated. The residue purified by column chromatography (Stationary phase: irregular SiOH 15-40 μm 80 g, gradient from 80% heptane, 20% EtOAc to 40% heptane, 50% EtOAc, 10% MeOH with 2% NH₄OH) to give Intermediate 309 (2.8 g, yield 46%).

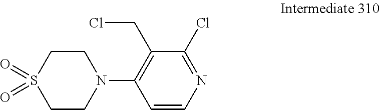 [SMILES: O=S1(=O)CCN(c2ccnc(Cl)c2CCl)CC1]

SOCl₂ (2.595 mL, 35.773 mmol, 3.0 eq.) was added to a stirred suspension of Intermediate 309 (3.3 g, 11.924 mmol) in DCM (2.6 mL) at room temperature. The reaction mixture was stirred at room temperature for 6 h. The volatiles were evaporated to give Intermediate 310 (3.52 g, quantitative yield).

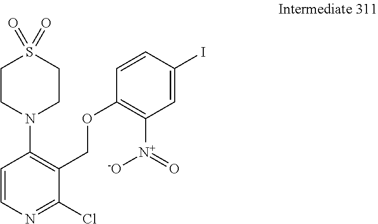

Intermediate 310 (3.5 g, 11.9 mmol), 4-iodo-2-nitrophenol [CAS: 21784-73-6] (3.77 g, 14.2 mmol, 1.2 eq.), and K₂CO₃ (4.92 g, 35.6 mmol, 3.0 eq.) in DMF (23 mL) were stirred at 80° C. for 12 h. The reaction mixture was poured into water, acidified with 3 N HCl aqueous solution, and extracted twice with EtOAc. The organic layer was dried over MgSO₄, filtered, and evaporated. The residue was purified by column chromatography over silica gel (SiO₂, 40 g; gradient from 80% DCM, 20% heptane to 96% DCM, 4% MeOH, 0.1% NH₄OH) to give Intermediate 311 (4.38 g, yield 71%).

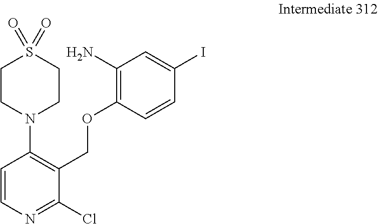 [SMILES: Nc1cc(I)ccc1OCc1c(N2CCS(=O)(=O)CC2)ccnc1Cl]

Intermediate 312 was synthesized in a similar manner as Intermediate 142 using Intermediate 311 instead of Intermediate 141.

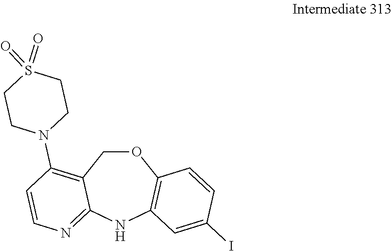 [SMILES: O=S1(=O)CCN(c2ccnc3c2COc2ccc(I)cc2N3)CC1]

Intermediate 313 was synthesized in a similar manner as Intermediate 3 using Intermediate 312 instead of Intermediate 2.

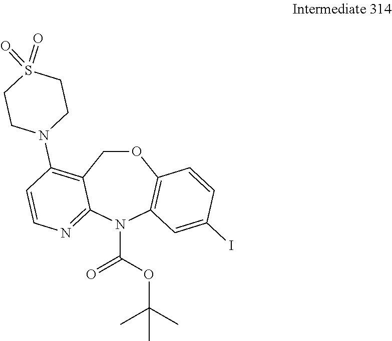 [SMILES: CC(C)(C)OC(=O)N1c2cc(I)ccc2OCc2c(N3CCS(=O)(=O)CC3)ccnc21]

Intermediate 314 was synthesized in a similar manner as Intermediate 202 using Intermediate 313 instead of Intermediate 201.

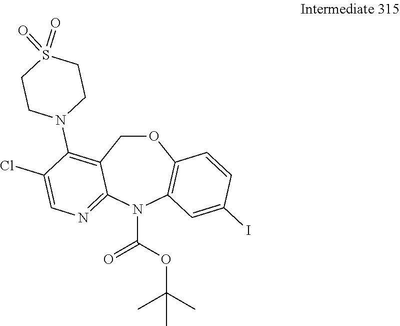 [SMILES: CC(C)(C)OC(=O)N1c2cc(I)ccc2OCc2c1ncc(Cl)c2N1CCS(=O)(=O)CC1]

Intermediate 315 was synthesized in a similar manner as Intermediate 176 using Intermediate 314 instead of Intermediate 175.

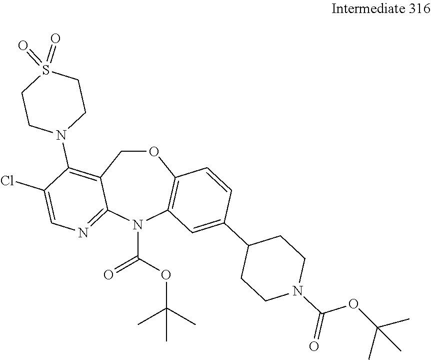 [SMILES: CC(C)(C)OC(=O)N1CCC(c2ccc3c(c2)N(C(=O)OC(C)(C)C)c2ncc(Cl)c(N4CCS(=O)(=O)CC4)c2CO3)CC1]

Intermediate 316 was synthesized in a similar manner as Intermediate 11 using Intermediate 315 instead of Intermediate 4.

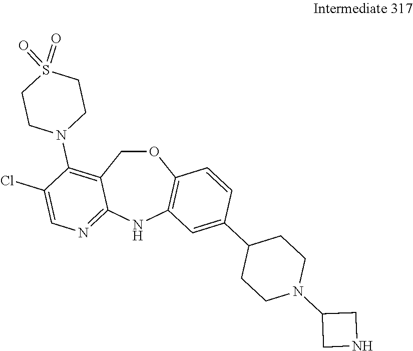 [SMILES: O=S1(=O)CCN(c2c(Cl)cnc3c2COc2ccc(C4CCN(C5CNC5)CC4)cc2N3)CC1]

Intermediate 317 was synthesized following the synthetic route of Intermediate 162 to Intermediate 164 starting with Intermediate 316 instead of Intermediate 161.

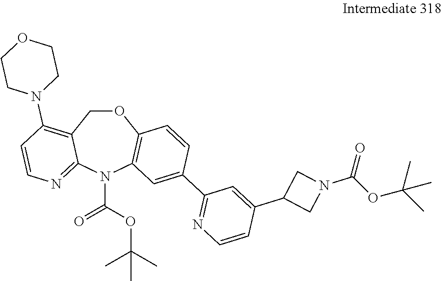 [SMILES: CC(C)(C)OC(=O)N1CC(c2ccnc(-c3ccc4c(c3)N(C(=O)OC(C)(C)C)c3nccc(N5CCOCC5)c3CO4)c2)C1]

Intermediate 318 was synthesized in a similar manner as Intermediate 20 using Intermediate 267 instead of N-Boc-1,2,3,6-tetrahydropyridine-4-boronic acid pinacol ester and Intermediate 303B instead of Intermediate 19.

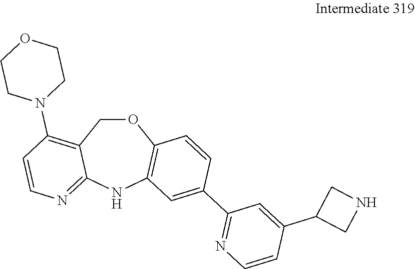 [SMILES: c1cc(C2CNC2)cc(-c2ccc3c(c2)Nc2nccc(N4CCOCC4)c2CO3)n1]

Intermediate 319 was synthesized in a similar manner as Intermediate 124 using Intermediate 318 instead of Intermediate 123.

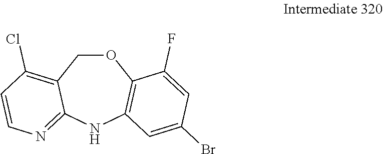 [SMILES: Fc1cc(Br)cc2c1OCc1c(Cl)ccnc1N2]

Intermediate 320 was synthesized following the synthetic route from Intermediate 1 to Intermediate 3 starting with 4-bromo-2-fluoro-6-nitrophenol [CAS: 320-76-3] instead of 4-bromo-2-nitrophenol.

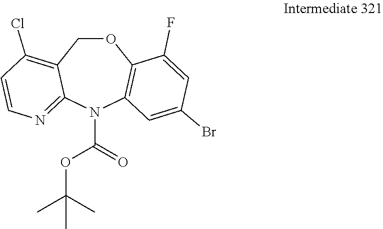 [SMILES: CC(C)(C)OC(=O)N1c2cc(Br)cc(F)c2OCc2c(Cl)ccnc21]

(Boc)₂O (5.9 g, 27.0 mmol, 2.0 eq.) was added to a solution of Intermediate 320 (6.0 g, 13.5 mmol, 1.0 eq.), DMAP (329 mg, 2.7 mmol, 0.2 eq.), and Et₃N (3.8 mL, 27.0 mmol, 2.0 eq.) in DCM (150 mL) and the reaction mixture was stirred at room temperature for 72 h. The reaction mixture was concentrated to dry and the residue was purified by flash column chromatography on silica gel (EtOAc-heptane gradient 5% to 50%) to afford Intermediate 321 (4.8 g, yield: 83%).

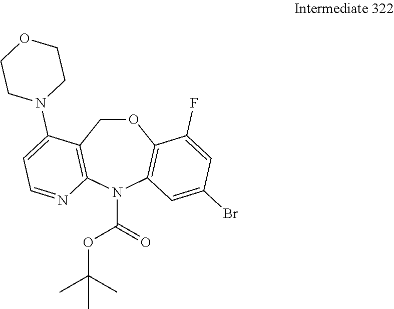 [SMILES: CC(C)(C)OC(=O)N1c2cc(Br)cc(F)c2OCc2c(N3CCOCC3)ccnc21]

Intermediate 322 was synthesized in a similar manner as Intermediate 179 using Intermediate 321 instead of Intermediate 143.

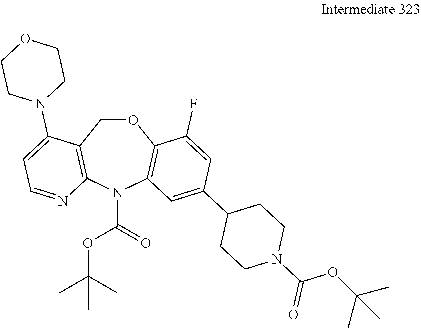 [SMILES: CC(C)(C)OC(=O)N1CCC(c2cc(F)c3c(c2)N(C(=O)OC(C)(C)C)c2nccc(N4CCOCC4)c2CO3)CC1]

Intermediate 323 was synthesized in a similar manner as Intermediate 211 using Intermediate 322 instead of Intermediate 210.

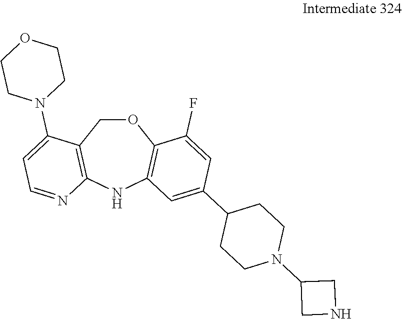 [SMILES: Fc1cc(C2CCN(C3CNC3)CC2)cc2c1OCc1c(N3CCOCC3)ccnc1N2]

Intermediate 324 was synthesized following the synthetic route from Intermediate 205 to Intermediate 207 starting from Intermediate 323 instead of Intermediate 204.

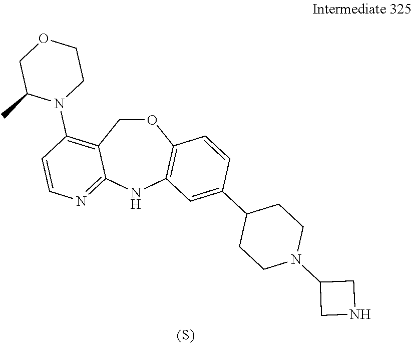 [SMILES: C[C@H]1COCCN1c1ccnc2c1COc1ccc(C3CCN(C4CNC4)CC3)cc1N2 ; "(S)"]

Intermediate 325 was synthesized following the synthetic route from Intermediate 12 to Intermediate 15 starting with (3S)-3-methylmorpholine [CAS: 350595-57-2] instead of morpholine.

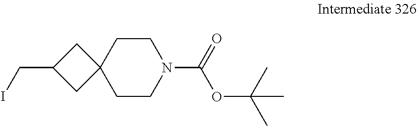 [SMILES: CC(C)(C)OC(=O)N1CCC2(CC1)CC(CI)C2]

Iodine (1.1 g, 4.4 mmol, 1.6 eq.) was added portionwise to a solution of 1,1-dimethylethyl 2-(hydroxymethyl)-7-azaspiro[3.5]nonane-7-carboxylate [CAS: 1356476-27-1], PPh₃ (1.16 g, 4.4 mmol, 1.6 eq.), and imidazole (376 mg, 5.5 mmol, 2.0 eq.) in THF (8 mL) at 0° C. The mixture was stirred for 3 h at room temperature. The reaction mixture was diluted with 10% aqueous Na₂SO₃ and was extracted with EtOAc. The organic layer was dried (Na₂SO₄), filtered, and the solvent evaporated. The residue was purified by column chromatography (Silica, DCM in heptane 50/50 to 100/0) to yield Intermediate 326 (426 mg, yield: 38%) as a colourless oil.

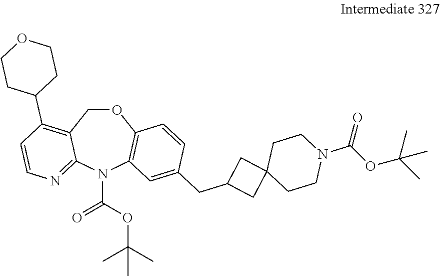 [SMILES: CC(C)(C)OC(=O)N1CCC2(CC1)CC(Cc1ccc3c(c1)N(C(=O)OC(C)(C)C)c1nccc(C4CCOCC4)c1CO3)C2]

A solution of Intermediate 326 (100 mg, 0.25 mmol, 5.0 eq.) in a lithium chloride solution (0.5 M in THF, 0.6 mL, 0.3 mmol, 6.0 eq.) was pumped through a column containing activated zinc (3.75 g) at 0.5 m/min and at 40° C. The output was collected in a vial containing Intermediate 231 (25 mg, 0.05 mmol), Pd(OAc)₂ (0.5 mg, 0.0025 mmol, 0.05 eq.), and CPhos (CAS [1160556-64-8], 2 mg, 0.005 mmol, 0.1 eq.) The reaction mixture was heated at 50° C. for 2 h. The excess of zincate was quenched with 10% aqueous NH₄Cl and 32% aqueous NH₄OH and the resulting mixture was extracted with EtOAc. The organic layer was dried (Na₂SO₄), filtered, and the solvent evaporated. The residue was purified by column chromatography (silica, EtOAc in DCM 0/100 to 75/25) to yield Intermediate 327 (14 mg, yield: 46%) as a yellow oil.

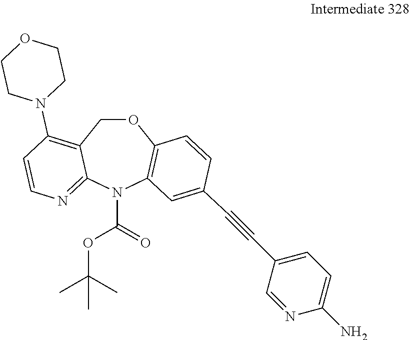 [SMILES: CC(C)(C)OC(=O)N1c2cc(C#Cc3ccc(N)nc3)ccc2OCc2c(N3CCOCC3)ccnc21]

Bistriphenylphosphine dichloro palladium (II) (CAS [13965-03-2], 76 mg, 0.11 mmol, 0.1 eq.) and copper (I) iodide (41 mg, 0.22 mmol, 0.2 eq.) were added to a solution of Intermediate 175 (500 mg, 1.1 mmol, 1.0 eq.), 5-ethynylpyridin-2-amine ([CAS: 82454-61-3], 153 mg, 1.3 mmol, 1.2 eq.), diisopropylamine (305 μL, 2.2 mmol, 2.0 eq.), and triphenylphosphine (57 mg, 0.22 mmol, 0.2 eq.) in DMF (3 mL) under nitrogen atmosphere. The mixture was stirred at 80° C. for 15 h. After cooling, the mixture was filtered, then diluted with water. The resulting solid was filtered, washed with plenty of water, and dried under high vacuum to afford Intermediate 328 (655 mg, yield: 61%) as a brown solid, used without further purification.

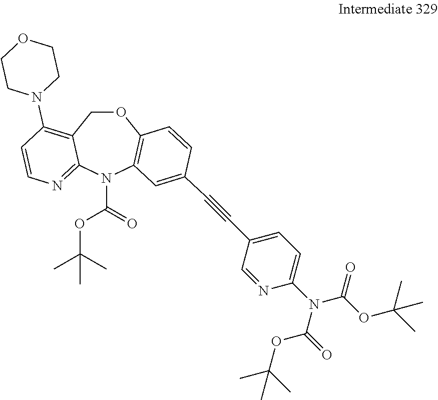 [SMILES: CC(C)(C)OC(=O)N(C(=O)OC(C)(C)C)c1ccc(C#Cc2ccc3c(c2)N(C(=O)OC(C)(C)C)c2nccc(N4CCOCC4)c2CO3)cn1]

Intermediate 329 was synthesized in a similar manner as Intermediate 321 using Intermediate 328 instead of Intermediate 320.

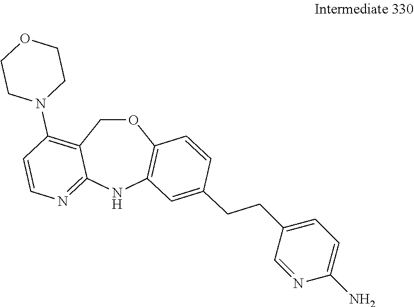 [SMILES: Nc1ccc(CCc2ccc3c(c2)Nc2nccc(N4CCOCC4)c2CO3)cn1]

Intermediate 330 was synthesized following the synthetic route from Intermediate 145 to Intermediate 146 starting with Intermediate 329 instead of Intermediate 144.

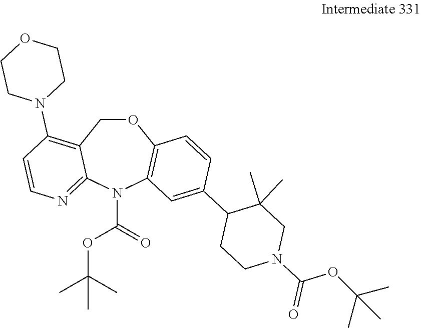 [SMILES: CC(C)(C)OC(=O)N1CCC(c2ccc3c(c2)N(C(=O)OC(C)(C)C)c2nccc(N4CCOCC4)c2CO3)C(C)(C)C1]

Intermediate 331 was synthesized following the synthetic route from Intermediate 29 to Intermediate 30 starting with Intermediate 267 instead of (1-tert-butoxycarbonyl-1,2,3,6-tetrahydropyridin-4-yl)boronic acid pinacol ester [CAS 286961-14-6] and tert-butyl 3,3-dimethyl-4-(((trifluoromethyl)sulfonyl)oxy)-3,6-dihydropyridine-1(2H)-carboxylate [CAS: 324769-08-6] instead of Intermediate 28.

### Intermediate 332A and Intermediate 332B

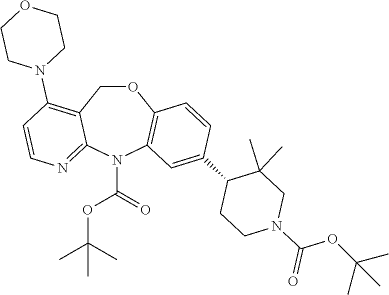 [SMILES: CC(C)(C)OC(=O)N1CC[C@H](c2ccc3c(c2)N(C(=O)OC(C)(C)C)c2nccc(N4CCOCC4)c2CO3)C(C)(C)C1]

Intermediate 332A: (*R), pure stereoisomer but absolute stereochemistry undetermined

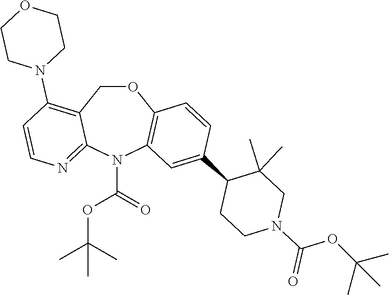 [SMILES: CC(C)(C)OC(=O)N1CC[C@@H](c2ccc3c(c2)N(C(=O)OC(C)(C)C)c2nccc(N4CCOCC4)c2CO3)C(C)(C)C1]

Intermediate 332B: (*S), pure stereoisomer but absolute stereochemistry undetermined Intermediate 331 (580 mg, 0.98 mmol) was separated into its enantiomers by chiral chromatography (Phenomenex Lux Amylose-1 250×30 mm 5 μm; gradient from 90% [heptane+0.1% DEA]−100% [iPrOH+0.1% DEA] to 54% [heptane+0.1% DEA]−46% [iPrOH+0.1% DEA]) to yield Intermediate 332A (242 mg, yield: 41%) and Intermediate 332B (224 mg, yield: 39%).

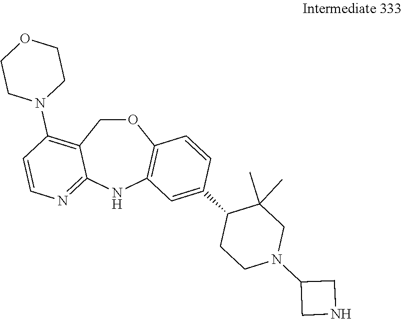 [SMILES: CC1(C)CN(C2CNC2)CC[C@@H]1c1ccc2c(c1)Nc1nccc(N3CCOCC3)c1CO2]

(*R), pure stereoisomer but absolute stereochemistry undetermined Intermediate 333 was synthesized following the synthetic route from Intermediate 23 to Intermediate 25 starting with Intermediate 332A instead of Intermediate 22.

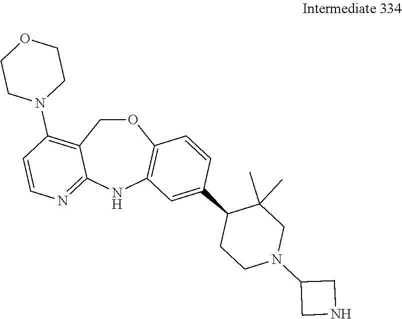 [SMILES: CC1(C)CN(C2CNC2)CC[C@H]1c1ccc2c(c1)Nc1nccc(N3CCOCC3)c1CO2]

(*S), pure stereoisomer but absolute stereochemistry undetermined Intermediate 334 was synthesized following the synthetic route from Intermediate 23 to Intermediate 25 starting with Intermediate 332B instead of Intermediate 22.

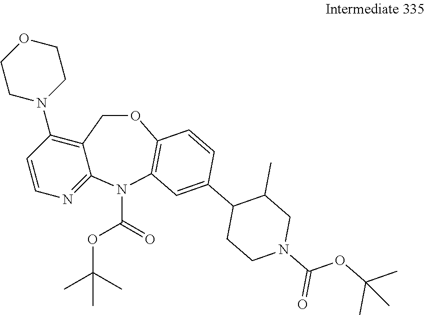 [SMILES: CC1CN(C(=O)OC(C)(C)C)CCC1c1ccc2c(c1)N(C(=O)OC(C)(C)C)c1nccc(N3CCOCC3)c1CO2]

Intermediate 335 was synthesized following the synthetic route from Intermediate 29 to Intermediate 30 starting with Intermediate 267 instead of (1-tert-butoxycarbonyl-1,2,3,6-tetrahydropyridin-4-yl)boronic acid pinacol ester and tert-butyl 3-methyl-4-(((trifluoromethyl)sulfonyl)oxy)-3,6-dihydropyridine-1(2H)-carboxylate [CAS: 1240971-20-3] instead of Intermediate 28.

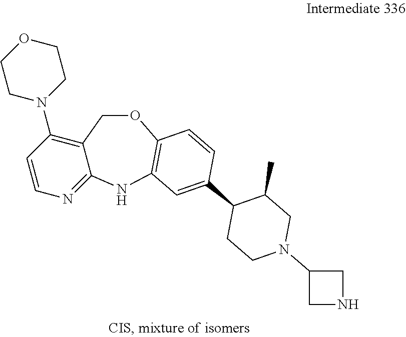 [SMILES: C[C@H]1CN(C2CNC2)CC[C@H]1c1ccc2c(c1)Nc1nccc(N3CCOCC3)c1CO2]

Intermediate 336 was synthesized following the synthetic route from Intermediate 23 to Intermediate 25 starting with Intermediate 335 instead of Intermediate 22.

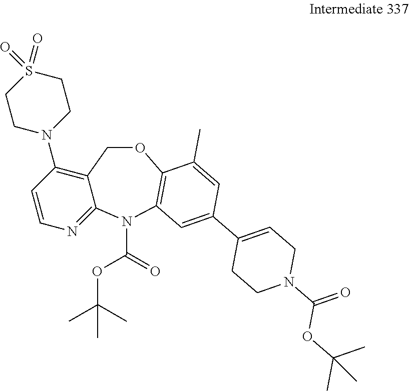 [SMILES: Cc1cc(C2=CCN(C(=O)OC(C)(C)C)CC2)cc2c1OCc1c(N3CCS(=O)(=O)CC3)ccnc1N2C(=O)OC(C)(C)C]

Intermediate 20 (3.2 g, 6.0 mmol, 1.0 eq.), thiomorpholine 1,1-dioxide ([CAS: 39093-93-1], 1.6 g, 12.0 mmol, 2.0 eq.), and sodium tert butoxide (1.15 g, 12.0 mmol, 2.0 eq.) were mixed in 1,4-dioxane (75 mL) and toluene (75 mL) and the mixture was degassed with nitrogen. Then X-Phos (571 mg, 1.2 mmol, 0.2 eq.) and Pd₂(dba)₃ (549 mg, 0.6 mmol, 0.1 eq.) were added and the mixture was degassed with nitrogen again. The reaction mixture was stirred at 100° C. for 6 h. The reaction mixture was partitioned between EtOAc and brine. The combined organic layers were concentrated. The residue was purified by column flash chromatography (silica; DCM-MeOH from 100/0 to 90/10) to afford Intermediate 337 (2.85 g, yield: 76%).

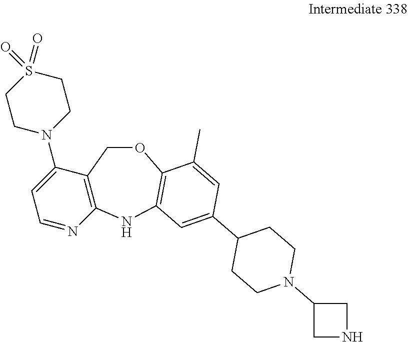 [SMILES: Cc1cc(C2CCN(C3CNC3)CC2)cc2c1OCc1c(N3CCS(=O)(=O)CC3)ccnc1N2]

Intermediate 338 was synthesized following the synthetic route from Intermediate 22 to Intermediate 25 starting with Intermediate 337 instead of Intermediate 21.

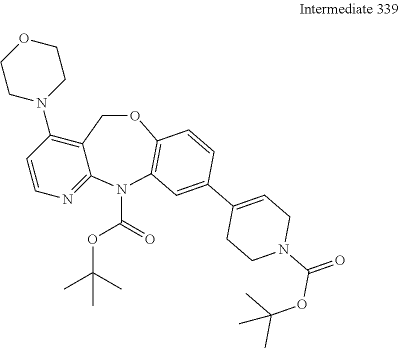 [SMILES: CC(C)(C)OC(=O)N1CC=C(c2ccc3c(c2)N(C(=O)OC(C)(C)C)c2nccc(N4CCOCC4)c2CO3)CC1]

Intermediate 339 was synthesized in a similar manner as Intermediate 5 using Intermediate 175 instead of Intermediate 4.

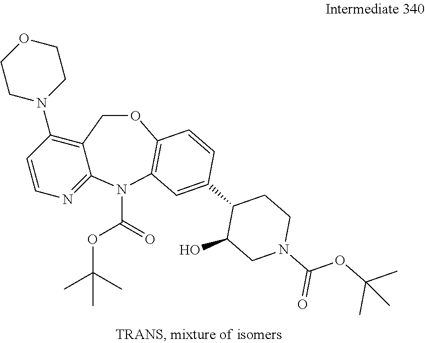 [SMILES: CC(C)(C)OC(=O)N1CC[C@@H](c2ccc3c(c2)N(C(=O)OC(C)(C)C)c2nccc(N4CCOCC4)c2CO3)[C@H](O)C1]

Borane tetrahydrofuran complex ([CAS: 14044-65-6], 17.4 mL, 2 M, 34.8 mmol, 4.0 eq.) was added dropwise to a solution of Intermediate 339 (5.3 g, 8.7 mmol, 1.0 eq.) in dry THF (220 mL) at room temperature. The reaction was stirred for 6 h before quenching by the addition of hydrogen peroxide (30% in water, 3.5 mL, 34.8 mmol, 4.0 eq.). NaOH (4 M in water, 97 mL) was added and the mixture was stirred at 65° C. for 2 h. Saturated aqueous Na₂S₂O₃ was added. The mixture was extracted with EtOAc. The organic layer was dried over MgSO₄, filtered, and concentrated. The residue was purified by column chromatography over silica gel (gradient of EtOAc in heptane from 0% to 70%) to yield Intermediate 340 (2.99 g, yield: 36%) as an oil.

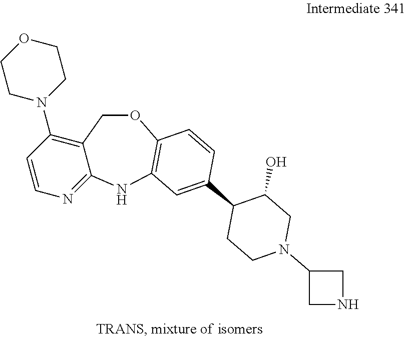 [SMILES: O[C@@H]1CN(C2CNC2)CC[C@H]1c1ccc2c(c1)Nc1nccc(N3CCOCC3)c1CO2]

Intermediate 341 was synthesized following the synthetic route from Intermediate 23 to Intermediate 25 starting with Intermediate 340 instead of Intermediate 22.

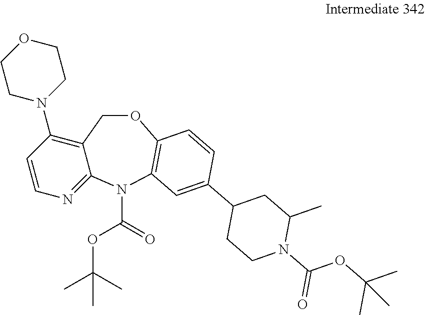 [SMILES: CC1CC(c2ccc3c(c2)N(C(=O)OC(C)(C)C)c2nccc(N4CCOCC4)c2CO3)CCN1C(=O)OC(C)(C)C]

Intermediate 342 was synthesized following the synthetic route from Intermediate 29 to Intermediate 30 starting with Intermediate 267 instead of (1-tert-butoxycarbonyl-1,2,3,6-tetrahydropyridin-4-yl)boronic acid pinacol ester and tert-butyl 2-methyl-4-(((trifluoromethyl)sulfonyl)oxy)-3,6-dihydropyridine-1(2H)-carboxylate (CAS [252563-92-1]) instead of Intermediate 28.

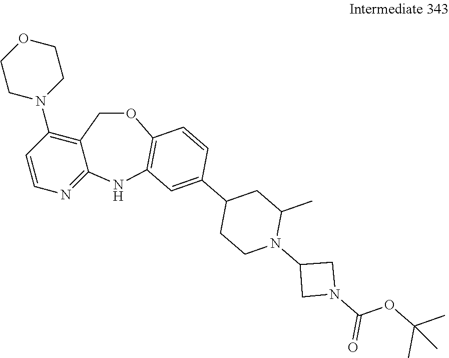 [SMILES: CC1CC(c2ccc3c(c2)Nc2nccc(N4CCOCC4)c2CO3)CCN1C1CN(C(=O)OC(C)(C)C)C1]

Intermediate 343 was synthesized following the synthetic route from Intermediate 23 to Intermediate 24 starting Intermediate 342 instead of Intermediate 22.

### Intermediate 344A and Intermediate 344B

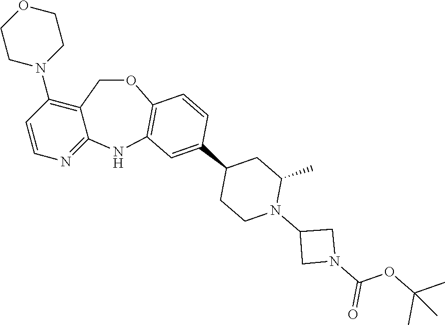 [SMILES: C[C@H]1C[C@H](c2ccc3c(c2)Nc2nccc(N4CCOCC4)c2CO3)CCN1C1CN(C(=O)OC(C)(C)C)C1]

Intermediate 344A (7*R, 11*S) pure isomer but absolute stereochemistry undetermined

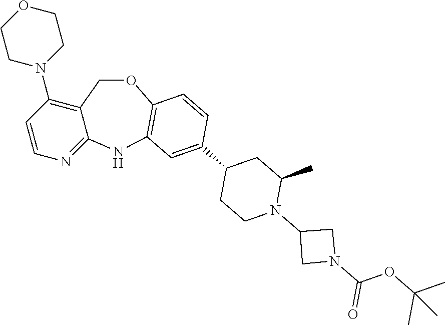 [SMILES: C[C@@H]1C[C@@H](c2ccc3c(c2)Nc2nccc(N4CCOCC4)c2CO3)CCN1C1CN(C(=O)OC(C)(C)C)C1]

Intermediate 344B (7*S, 11*R) pure isomer but absolute stereochemistry undetermined Intermediate 343 (578 mg, 1.08 mmol) was separated by chiral chromatography (Phenomenex Lux Cellulose-1 250×30 mm 5 μm; gradient from 70% [heptane+0.1% DEA]−30% [iPrOH-EtOH (9:1)+0.1% DEA] to 27% [heptane+0.1% DEA]−73% [iPrOH-EtOH (9:1)+0.1% DEA]) to yield Intermediate 344A (237 mg, 41%) and Intermediate 344B (287 mg, 50%).

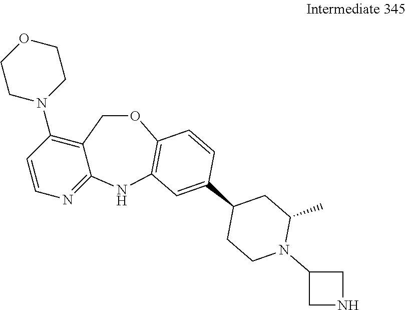 [SMILES: C[C@H]1C[C@H](c2ccc3c(c2)Nc2nccc(N4CCOCC4)c2CO3)CCN1C1CNC1]

(7*R, 11*S) pure isomer but absolute stereochemistry undetermined Intermediate 345 was synthesized in a similar manner as Intermediate 25 using Intermediate 344A instead of Intermediate 24.

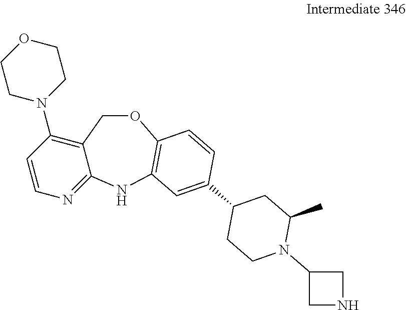 [SMILES: C[C@@H]1C[C@@H](c2ccc3c(c2)Nc2nccc(N4CCOCC4)c2CO3)CCN1C1CNC1]

(7*S, 11*R) pure isomer but absolute stereochemistry undetermined Intermediate 346 was synthesized in a similar manner as Intermediate 25 using Intermediate 344B instead of Intermediate 24.

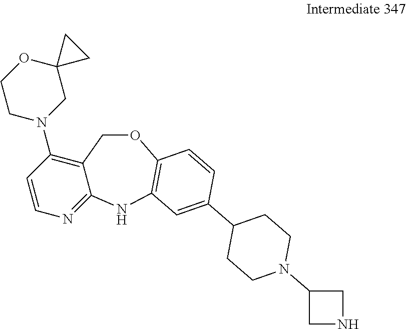 [SMILES: c1cc(N2CCOC3(CC3)C2)c2c(n1)Nc1cc(C3CCN(C4CNC4)CC3)ccc1OC2]

Intermediate 347 was synthesized following the synthetic route from Intermediate 12 to Intermediate 15 starting with 4-oxa-7-azaspiro[2.5]octane hydrochloride [CAS: 1427195-23-0] instead of morpholine.

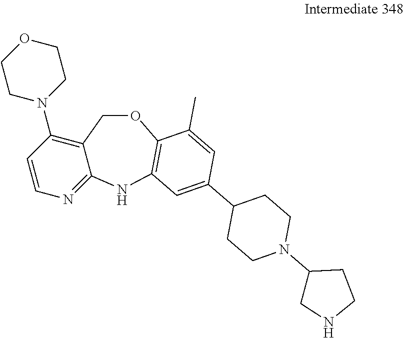 [SMILES: Cc1cc(C2CCN(C3CCNC3)CC2)cc2c1OCc1c(N3CCOCC3)ccnc1N2]

Intermediate 348 was synthesized following the synthetic route from Intermediate 163 to Intermediate 164 starting with Intermediate 23 instead of Intermediate 162 and pyrrolidin-3-one, N-Boc protected [CAS: 101385-93-7] instead of 1-Boc-3-azetidinone.

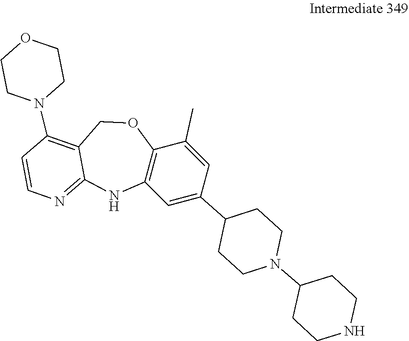 [SMILES: Cc1cc(C2CCN(C3CCNCC3)CC2)cc2c1OCc1c(N3CCOCC3)ccnc1N2]

Intermediate 349 was synthesized following the synthetic route from Intermediate 163 to Intermediate 164 starting with Intermediate 23 instead of Intermediate 162 and piperidin-4-one, N-Boc protected [CAS: 79099-07-3] instead of 1-Boc-3-azetidinone.

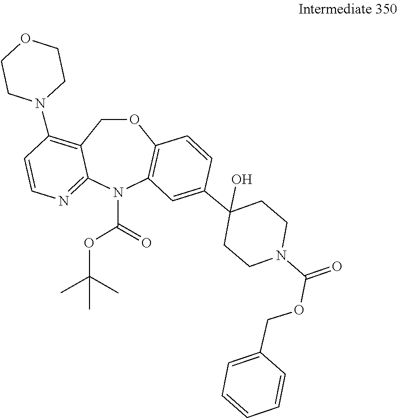 [SMILES: CC(C)(C)OC(=O)N1c2cc(C3(O)CCN(C(=O)OCc4ccccc4)CC3)ccc2OCc2c(N3CCOCC3)ccnc21]

nBuLi (2.5 M in hexanes, 1.13 mL, 2.8 mmol, 1.0 eq.) was added slowly to a solution of Intermediate 175 (1.3 g, 2.8 mmol, 1.0 eq.) in dry THF (35 mL) under nitrogen atmosphere at −78° C. The mixture was stirred at −78° C. for 30 min. A mixture of benzyl 4-oxo-1-piperidinecarboxylate ([CAS: 19099-93-5], 787 mg, 3.4 mmol, 1.2 eq.) in dry THF (15 mL) was added. The reaction mixture was allowed to warm up to room temperature. Saturated aqueous NH₄Cl was added. The mixture was extracted with EtOAc. The organic layer was dried over MgSO₄, filtered, and concentrated. The residue was purified by column chromatography over silica gel (gradient of EtOAc in heptane from 0% to 60%) to yield Intermediate 350 (730 mg, yield: 42%) as an oil.

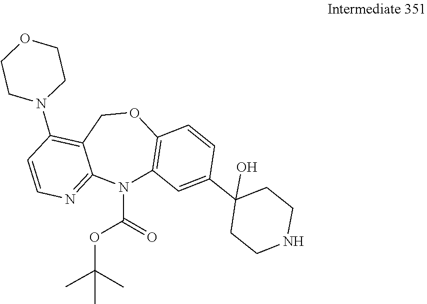 [SMILES: CC(C)(C)OC(=O)N1c2cc(C3(O)CCNCC3)ccc2OCc2c(N3CCOCC3)ccnc21]

A solution of Intermediate 350 (708 mg, 1.148 mmol) in MeOH (15 mL) was cooled to 0° C. under nitrogen atmosphere. 10% Pd/C (74 mg, 0.697 mmol, 0.6 eq.) was added and the reaction vessel was connected to a balloon filled with hydrogen. The reaction mixture was stirred under atmosphere of hydrogen overnight at room temperature. The catalyst was filtered off and the filtrate was concentrated to give Intermediate 351 (508 mg, yield: 88%) as a foam, used without further purification.

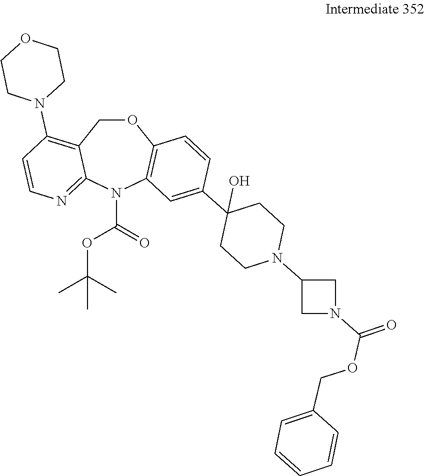 [SMILES: CC(C)(C)OC(=O)N1c2cc(C3(O)CCN(C4CN(C(=O)OCc5ccccc5)C4)CC3)ccc2OCc2c(N3CCOCC3)ccnc21]

Intermediate 352 was synthesized in a similar manner as Intermediate 163 using Intermediate 351 instead of Intermediate 162 and benzyl 3-oxoazetidine-1-carboxylate [CAS: 105258-93-3] instead of 1-Boc-3-azetidinone.

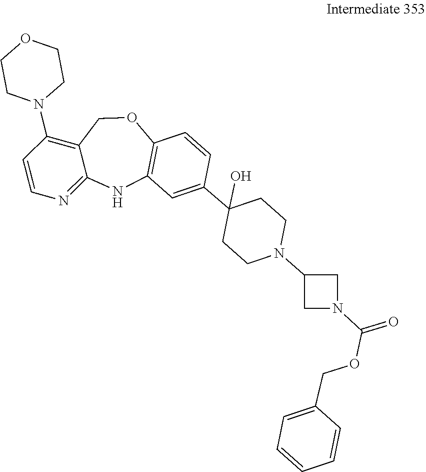 [SMILES: O=C(OCc1ccccc1)N1CC(N2CCC(O)(c3ccc4c(c3)Nc3nccc(N5CCOCC5)c3CO4)CC2)C1]

HCl (3 M in water, 3.4 mL, 10.3 mmol, 10.0 eq.) was added to a mixture of Intermediate 352 (837 mg, 1.03 mmol, 1.0 eq.) in MeOH (10 mL). The reaction mixture was stirred at room temperature and then HCl (37%, 12 M in water, 3.0 mL, 36.0 mmol, 34.8 eq.) was added. The reaction mixture was stirred for 24 h at room temperature. Aqueous Na₂CO₃ (1 M) and solid Na₂CO₃ were added until a basic pH was reached. DCM was added. The organic layer was separated, dried over MgSO₄, filtered, concentrated, and purified by column chromatography over silica gel (gradient of DCM/MeOH in DCM from 0% to 100%) to afford Intermediate 353 (260 mg, yield: 44%).

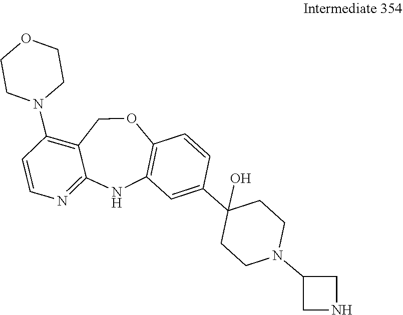 [SMILES: OC1(c2ccc3c(c2)Nc2nccc(N4CCOCC4)c2CO3)CCN(C2CNC2)CC1]

Intermediate 354 was synthesized in a similar manner as Intermediate 351 starting with Intermediate 353 instead of Intermediate 350.

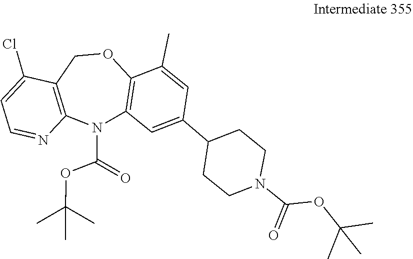 [SMILES: Cc1cc(C2CCN(C(=O)OC(C)(C)C)CC2)cc2c1OCc1c(Cl)ccnc1N2C(=O)OC(C)(C)C]

Intermediate 355 was synthesized in a similar manner as Intermediate 211 using Intermediate 19 instead of Intermediate 210.

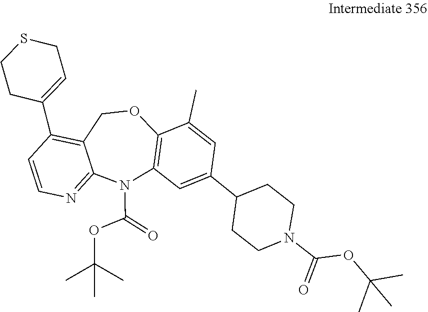 [SMILES: Cc1cc(C2CCN(C(=O)OC(C)(C)C)CC2)cc2c1OCc1c(C3=CCSCC3)ccnc1N2C(=O)OC(C)(C)C]

In a sealed tube, a solution of Intermediate 355 (795 mg, 1.485 mmol), dihydrothiopyran-4-boronic acid pinacol ester (CAS [862129-81-5], 672 mg, 2.97 mmol, 2 eq.), and potassium phosphate (630 mg, 2.97 mmol, 2 eq.) in 1,4-dioxane (10.5 mL) and water (1.5 mL) was degassed with nitrogen. Dichloro[1,1′-bis(diphenylphosphino)ferrocene]palladium(II), complex with dichloromethane (1:1) (CAS [95464-05-4], 122 mg, 0.148 mmol, 0.1 eq.) was added. The reaction mixture was degassed again with nitrogen and was stirred at 100° C. for 14 h. The reaction mixture was partitioned between EtOAc and brine. The combined organic layers were dried (MgSO₄), filtered, and concentrated. The residue was purified by flash chromatography (silica; heptane/EtOAc gradient) to afford Intermediate 356 (347 mg, yield: 37%) and unreacted Intermediate 355 (351 mg, yield: 44%).

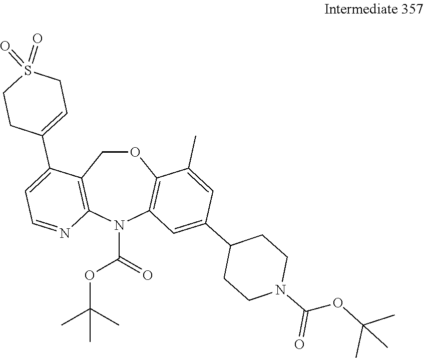 [SMILES: Cc1cc(C2CCN(C(=O)OC(C)(C)C)CC2)cc2c1OCc1c(C3=CCS(=O)(=O)CC3)ccnc1N2C(=O)OC(C)(C)C]

Intermediate 356 (444 mg, 0.75 mmol, 1.0 eq.) was dissolved in a mixture of acetone (4 mL) and aqueous acetic acid (20% in water, 2 mL). The reaction mixture was cooled to 0° C. and potassium permanganate (298 mg, 1.9 mmol, 2.5 eq.) was added. The reaction mixture was stirred at room temperature overnight. The mixture was poured into a solution of aqueous sodium sulfite and ice, and the mixture was stirred at the same temperature for 5 min. NaHCO₃ was added until a basic pH was reached. The reaction mixture was extracted with DCM. The organic layer was concentrated to afford Intermediate 357 (356 mg, yield: 76%).

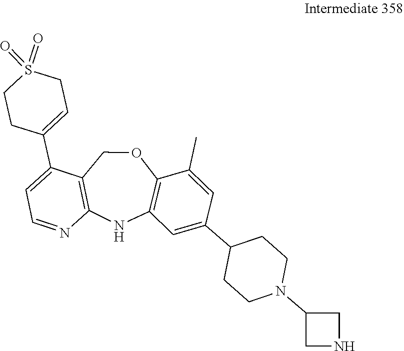 [SMILES: Cc1cc(C2CCN(C3CNC3)CC2)cc2c1OCc1c(C3=CCS(=O)(=O)CC3)ccnc1N2]

Intermediate 358 was synthesized following the synthetic route from Intermediate 23 to Intermediate 25 starting Intermediate 357 instead of Intermediate 22.

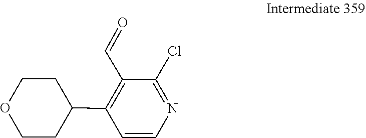 [SMILES: O=Cc1c(C2CCOCC2)ccnc1Cl]

2-Chloro-4-iodo-3-pyridinecarboxaldehyde [CAS: 153034-90-3] (10 g, 37.4 mmol, 1.0 eq.) and Pd(PPh₃)₄ (1.27 g, 1.1 mmol, 0.03 eq.) were dissolved in DMA (50 mL) under nitrogen atmosphere. A solution of iodo(tetrahydro-2H-pyran-4-yl)zinc [CAS: 1350356-52-3] (100 mL, 0.47 M in DMA, 47.0 mmol, 1.3 eq.) was added and the resulting mixture was stirred at 60° C. for 3 h. After cooling, the reaction was quenched with water (10 mL) and the mixture was concentrated under reduced pressure. EtOAc (300 mL), water (150 mL), and brine (20 mL) were added to the residue. The organic layer was separated, dried over MgSO₄, filtered, and concentrated. The residue was purified by column chromatography over silica gel (gradient of EtOAc in heptane from 0 to 5 0%) to give Intermediate 359 (3.5 g, yield: 41%) as an oil that crystallized.

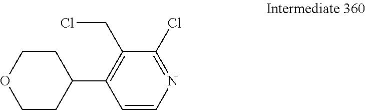 [SMILES: ClCc1c(C2CCOCC2)ccnc1Cl]

Intermediate 360 was synthesized following the synthetic route from Intermediate 309 to Intermediate 310 starting with Intermediate 359 instead of Intermediate 308.

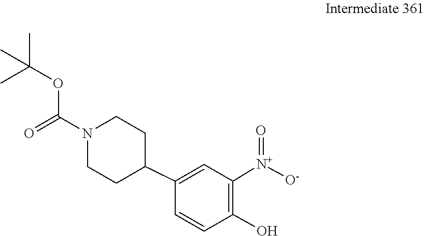 [SMILES: CC(C)(C)OC(=O)N1CCC(c2ccc(O)c([N+](=O)[O-])c2)CC1]

tert-Butyl 4-(4-hydroxyphenyl)piperidine-1-carboxylate [CAS: 149377-19-5] (5 g, 18.0 mmol, 1.0 eq.) was dissolved in acetic acid (20 mL) and DCM (2.5 mL). Nitric acid (65% in water, 1.3 mL, 19.4 mmol, 1.1 eq.) in acetic acid (5 mL) and water (50 μL) was added dropwise and the resulting mixture was stirred for 3 h. The reaction mixture was diluted with DCM (250 mL) and water (400 mL) was added. The organic layer was separated, washed with water (200 mL), dried over MgSO₄, filtered, and concentrated. The residue was purified by column chromatography over silica gel (gradient of EtOAc in heptane from 0 to 30%) to afford Intermediate 361 (3.5 g, yield: 56%) as a light yellow oil that crystallized upon standing.

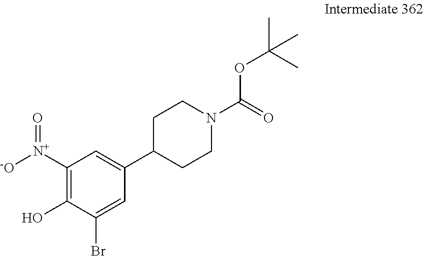

A solution of bromine (59 μL, 1.145 mmol) in AcOH (10 mL) was added dropwise to a solution of Intermediate 361 (3.5 g, 10.9 mmol) in AcOH (40 mL). Once the addition was complete, the mixture was stirred at room temperature. When a solid started to appear, MeOH (40 mL) was added to keep an homogenous solution. The reaction was continued for 5 h. The reaction mixture was diluted with DCM (200 mL) and washed with water (2×200 mL). The organic layer was dried over MgSO₄, filtered, and concentrated. The residue was dissolved in THF (25 mL) and water (15 mL). The pH was brought to 7-8 with 1 M aqueous Na₂CO₃. Di-tert-butyl dicarbonate (1.2 g, 5.4 mmol, 0.5 eq.) was added and the mixture was vigorously stirred for 4 h. The pH was then brought to 5-6 with 1 M KHSO₄. The reaction mixture was extracted with DCM (2×150 mL), dried over MgSO₄, filtered, and concentrated. The residue was purified via column chromatography over silica gel (gradient of EtOAc in heptane from 0 to 30%) to afford Intermediate 362 (2.6 g, yield: 54%).

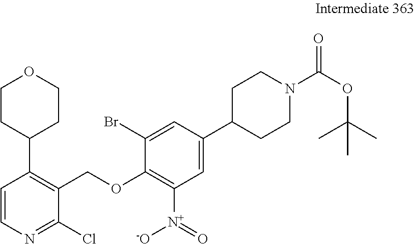

Intermediate 363 was synthesized in a similar manner as Intermediate 311 using Intermediate 360 instead of Intermediate 310 and Intermediate 362 instead of 4-iodo-2-nitrophenol.

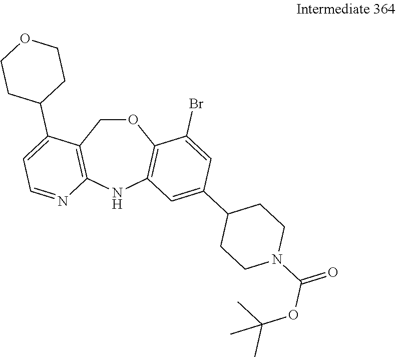 [SMILES: CC(C)(C)OC(=O)N1CCC(c2cc(Br)c3c(c2)Nc2nccc(C4CCOCC4)c2CO3)CC1]

Intermediate 364 was synthesized following the synthetic route from Intermediate 191 to Intermediate 192 starting Intermediate 363 instead of Intermediate 190.

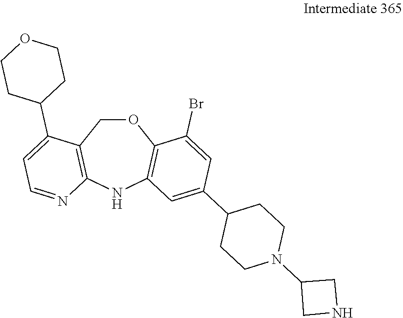 [SMILES: Brc1cc(C2CCN(C3CNC3)CC2)cc2c1OCc1c(C3CCOCC3)ccnc1N2]

Intermediate 365 was synthesized following the synthetic route from Intermediate 23 to Intermediate 25 starting with Intermediate 364 instead of Intermediate 22.

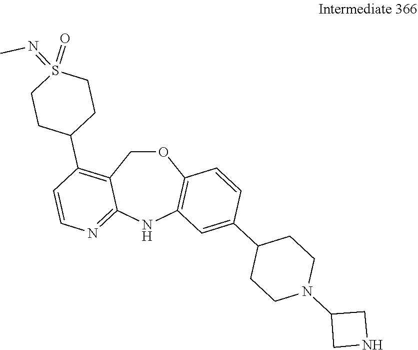 [SMILES: CN=S1(=O)CCC(c2ccnc3c2COc2ccc(C4CCN(C5CNC5)CC4)cc2N3)CC1]

Intermediate 366 was synthesized following the synthetic route from Intermediate 12 to Intermediate 15 starting with N-(1-Oxido-1λ⁴-thiomorpholin-1-ylidene)methanamine [CAS: 1621962-34-2] instead of morpholine.

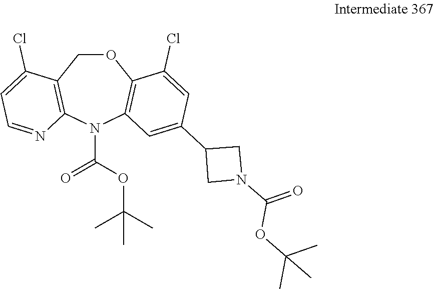 [SMILES: CC(C)(C)OC(=O)N1CC(c2cc(Cl)c3c(c2)N(C(=O)OC(C)(C)C)c2nccc(Cl)c2CO3)C1]

Intermediate 367 was synthesized in a similar manner as Intermediate 301 using Intermediate 227 instead of Intermediate 300.

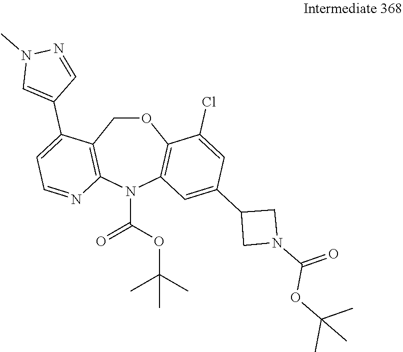 [SMILES: Cn1cc(-c2ccnc3c2COc2c(Cl)cc(C4CN(C(=O)OC(C)(C)C)C4)cc2N3C(=O)OC(C)(C)C)cn1]

Intermediate 367 (1.963 g, 3.758 mmol) and 1-methylpyrazole-4-boronic acid pinacol ester (CAS [761446-44-0], 1.172 g, 5.636 mmol, 1.5 eq.) were dissolved in 1,4-dioxane (20 mL) and aqueous Na₂CO₃ (1 M, 7.515 ml, 7.515 mmol, 2 eq.) and nitrogen was bubbled into the mixture for 10 min. Pd(dppf)Cl₂·DCM (CAS [95464-05-4], 307 mg, 0.376 mmol, 0.1 eq.) was added and the reaction mixture was stirred at 80° C. under nitrogen atmosphere for 24 h. More 1-methylpyrazole-4-boronic acid pinacol ester (CAS [761446-44-0], 1.172 g, 5.636 mmol, 1.5 eq.) was added and nitrogen was bubbled into the mixture for 10 min, then Pd(dppf)Cl₂·DCM (CAS [95464-05-4], 307 mg, 0.376 mmol, 0.1 eq.) was added and the reaction mixture was stirred at reflux under nitrogen atmosphere for 10 h. After cooling, the mixture was diluted with EtOAc (100 mL) and aqueous Na₂CO₃ (1 M, 50 mL). The mixture was filtered through a pad of celite and the cake was washed with EtOAc (3×30 mL). The organic layer was washed with brine (50 mL), dried over MgSO₄, filtered, and evaporated. The residue was purified by flash column chromatography (silica gel, EtOAc/heptane, from 0/100 to 50/50) to give Intermediate 368 (1.6 g, 74% pure, yield: 55%) as a brown foam.

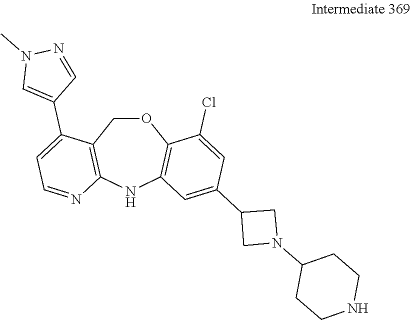 [SMILES: Cn1cc(-c2ccnc3c2COc2c(Cl)cc(C4CN(C5CCNCC5)C4)cc2N3)cn1]

Intermediate 369 was synthesized following the synthetic route from Intermediate 23 to Intermediate 25 starting from Intermediate 368 instead of Intermediate 22 and using tert-butyl 4-oxopiperidine-1-carboxylate [CAS: 79099-07-3] instead of N-Boc-3-oxoazetidine.

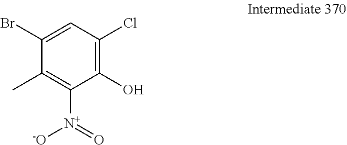

Intermediate 370 was synthesized in a similar manner as Intermediate 361 using 4-bromo-2-chloro-5-methylphenol [319473-24-0] instead of tert-Butyl 4-(4-hydroxyphenyl)piperidine-1-carboxylate.

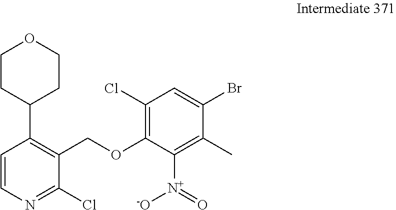

Intermediate 371 was synthesized in a similar manner as Intermediate 311 using Intermediate 360 instead of Intermediate 310 and Intermediate 370 instead of 4-iodo-2-nitrophenol.

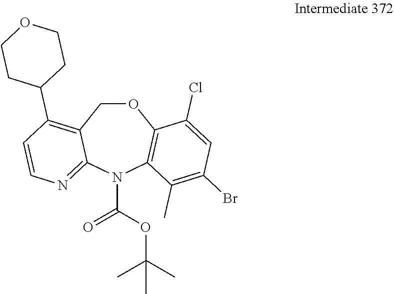 [SMILES: Cc1c(Br)cc(Cl)c2c1N(C(=O)OC(C)(C)C)c1nccc(C3CCOCC3)c1CO2]

Intermediate 372 was synthesized following the synthetic route from Intermediate 17 to Intermediate 19 starting Intermediate 371 instead of Intermediate 16.

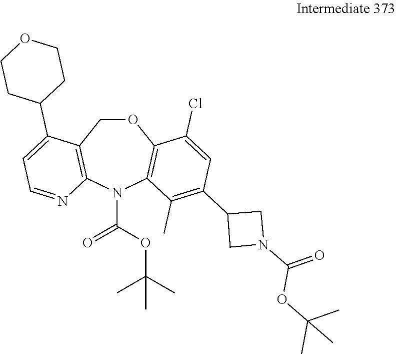 [SMILES: Cc1c(C2CN(C(=O)OC(C)(C)C)C2)cc(Cl)c2c1N(C(=O)OC(C)(C)C)c1nccc(C3CCOCC3)c1CO2]

Intermediate 373 was synthesized in a similar manner as Intermediate 301 using Intermediate 372 instead of Intermediate 300.

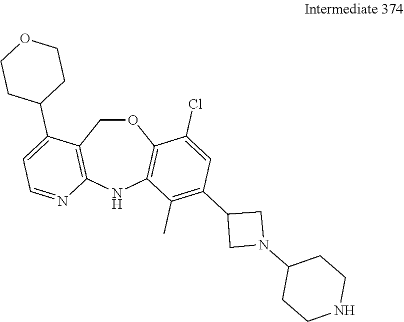 [SMILES: Cc1c(C2CN(C3CCNCC3)C2)cc(Cl)c2c1Nc1nccc(C3CCOCC3)c1CO2]

Intermediate 374 was synthesized following the synthetic route from Intermediate 23 to Intermediate 25 starting with Intermediate 373 instead of Intermediate 22 and using tert-butyl 4-oxopiperidine-1-carboxylate [CAS: 79099-07-3] instead of N-Boc-3-oxoazetidine.

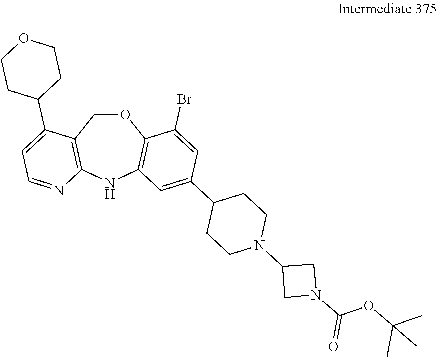 [SMILES: CC(C)(C)OC(=O)N1CC(N2CCC(c3cc(Br)c4c(c3)Nc3nccc(C5CCOCC5)c3CO4)CC2)C1]

Intermediate 375 was synthesized following the synthetic route from Intermediate 23 to Intermediate 24 starting with Intermediate 364 instead of Intermediate 22.

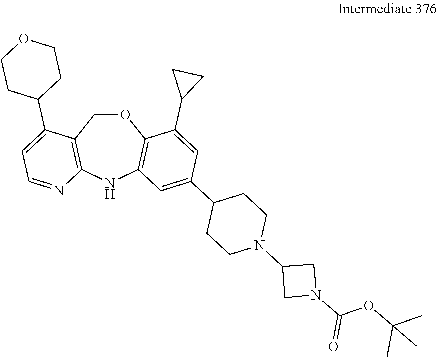 [SMILES: CC(C)(C)OC(=O)N1CC(N2CCC(c3cc4c(c(C5CC5)c3)OCc3c(C5CCOCC5)ccnc3N4)CC2)C1]

Intermediate 376 was synthesized in a similar manner as Intermediate 135 using Intermediate 375 instead of Intermediate 127.

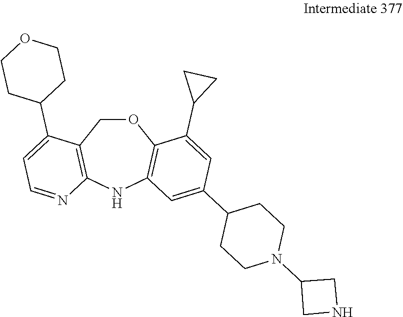 [SMILES: c1cc(C2CCOCC2)c2c(n1)Nc1cc(C3CCN(C4CNC4)CC3)cc(C3CC3)c1OC2]

Intermediate 377 was synthesized in a similar manner as Intermediate 25 using Intermediate 376 instead of Intermediate 24.

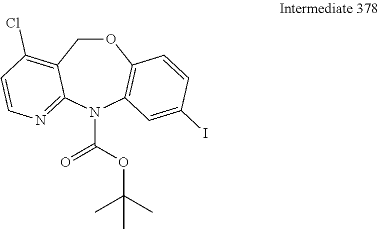 [SMILES: CC(C)(C)OC(=O)N1c2cc(I)ccc2OCc2c(Cl)ccnc21]

Intermediate 378 was synthesized in a similar manner as Intermediate 19 using Intermediate 155 instead of Intermediate 18.

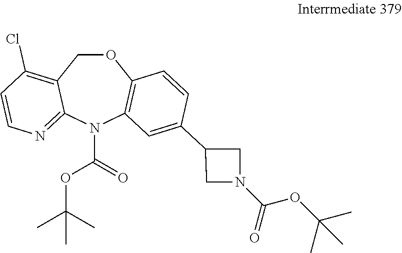 [SMILES: CC(C)(C)OC(=O)N1CC(c2ccc3c(c2)N(C(=O)OC(C)(C)C)c2nccc(Cl)c2CO3)C1]

Intermediate 379 was synthesized in a similar manner as Intermediate 177 using Intermediate 378 instead of Intermediate 176.

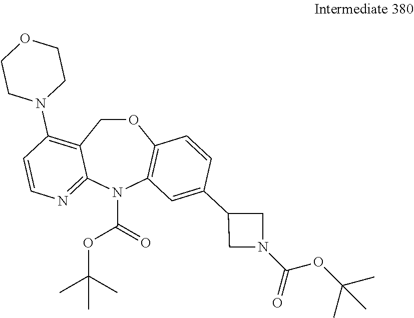 [SMILES: CC(C)(C)OC(=O)N1CC(c2ccc3c(c2)N(C(=O)OC(C)(C)C)c2nccc(N4CCOCC4)c2CO3)C1]

Intermediate 380 was synthesized in a similar manner as Intermediate 12 using Intermediate 379 instead of Intermediate 11 and [1-[(1,1-dimethylethoxy)carbonyl]-3-azetidinyl]iodozine [CAS: 206446-38-0] instead of [1-[(1,1-dimethylethoxy)carbonyl]-4-piperidinyl]iodozine.

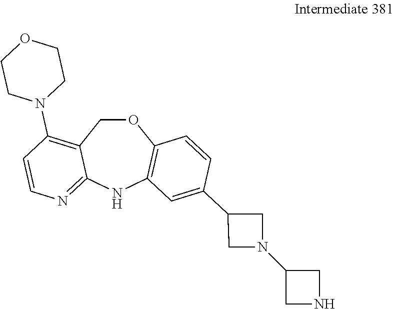 [SMILES: c1cc(N2CCOCC2)c2c(n1)Nc1cc(C3CN(C4CNC4)C3)ccc1OC2]

Intermediate 381 was synthesized following the synthetic route of Intermediate 162 to Intermediate 164 starting with Intermediate 380 instead of Intermediate 161.

### Intermediate 386A and Intermediate 386B

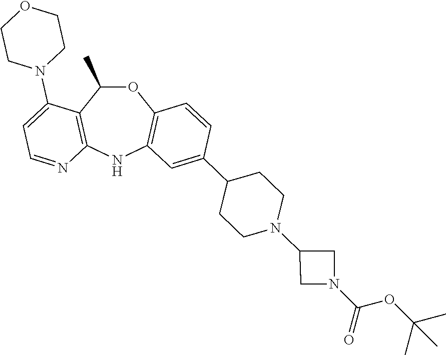 [SMILES: C[C@H]1Oc2ccc(C3CCN(C4CN(C(=O)OC(C)(C)C)C4)CC3)cc2Nc2nccc(N3CCOCC3)c21]

Intermediate 386A: (*R), pure stereoisomer but absolute stereochemistry undetermined

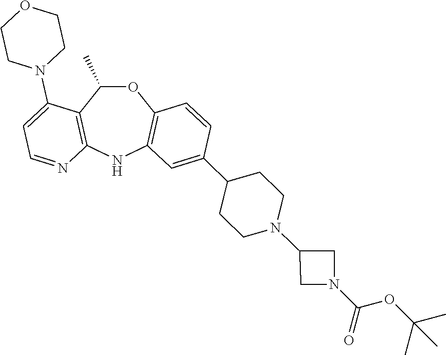 [SMILES: C[C@@H]1Oc2ccc(C3CCN(C4CN(C(=O)OC(C)(C)C)C4)CC3)cc2Nc2nccc(N3CCOCC3)c21]

Intermediate 386B3: (*S), pure stereoisomer but absolute stereochemistry undetermined Intermediate 206 (830 mg, 1.5 mmol) was separated into its enantiomers by chiral chromatography (Phenomenex Lux Cellulose-1 250×30 mm 5 μm; gradient from 75% [heptane+0.1% DEA]−25% [iPrOH+0.1% DEA] to 100% [iPrOH+0.1% DEA]) to afford Intermediate 386A (380 mg, yield: 46%) and Intermediate 386B (363 mg, yield: 44%).

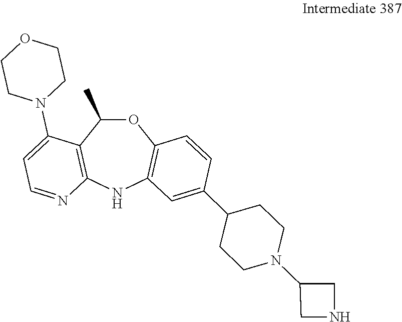 [SMILES: C[C@H]1Oc2ccc(C3CCN(C4CNC4)CC3)cc2Nc2nccc(N3CCOCC3)c21]

(*R), pure stereoisomer but absolute stereochemistry undetermined

Intermediate 387 was synthesized in a similar manner as Intermediate 23 using Intermediate 386A instead of Intermediate 22.

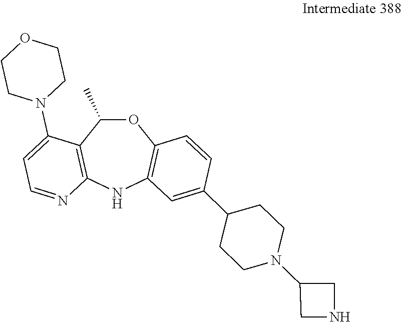 [SMILES: C[C@@H]1Oc2ccc(C3CCN(C4CNC4)CC3)cc2Nc2nccc(N3CCOCC3)c21]

(*S), pure stereoisomer but absolute stereochemistry undetermined Intermediate 388 was synthesized in a similar manner as Intermediate 23 using Intermediate 386B instead of Intermediate 22.

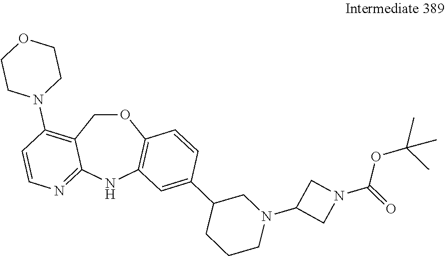 [SMILES: CC(C)(C)OC(=O)N1CC(N2CCCC(c3ccc4c(c3)Nc3nccc(N5CCOCC5)c3CO4)C2)C1]

Intermediate 389 was synthesized following the synthetic route from Intermediate 22 to Intermediate 24 starting with Intermediate 265 instead of Intermediate 21.

### Intermediate 390A and Intermediate 390B

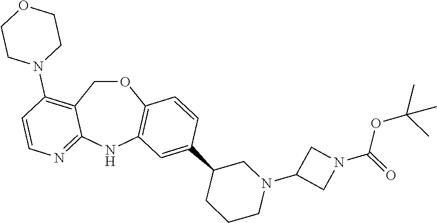 [SMILES: CC(C)(C)OC(=O)N1CC(N2CCC[C@@H](c3ccc4c(c3)Nc3nccc(N5CCOCC5)c3CO4)C2)C1]

Intermediate 390A: (*S), pure stereoisomer but absolute stereochemistry undetermined

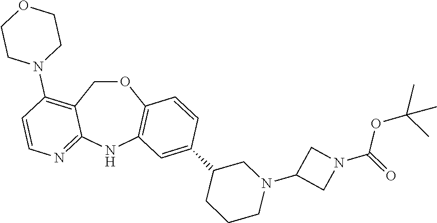

Intermediate 390B: (*R), pure stereoisomer but absolute stereochemistry undetermined

Intermediate 389 (660 mg, 1.27 mmol) was separated into its enantiomers by chiral SFC (Stationary phase: CHIRACEL OJ-H 5 μm 250*30 mm, Mobile phase: 60% CO₂, 40% EtOH (0.6% iPrNH₂)) to yield Intermediate 390A (275 mg, yield: 42%) and Intermediate 390B (287 mg, yield: 43%).

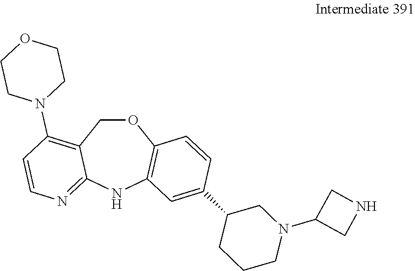 [SMILES: c1cc(N2CCOCC2)c2c(n1)Nc1cc([C@H]3CCCN(C4CNC4)C3)ccc1OC2]

(*R), pure stereoisomer but absolute stereochemistry undetermined Intermediate 391 was synthesized in a similar manner as Intermediate 164 using Intermediate 390B instead of Intermediate 163.

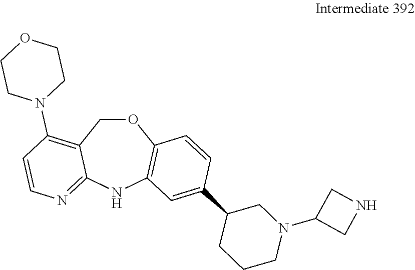 [SMILES: c1cc(N2CCOCC2)c2c(n1)Nc1cc([C@@H]3CCCN(C4CNC4)C3)ccc1OC2]

(*S), pure stereoisomer but absolute stereochemistry undetermined Intermediate 392 was synthesized in a similar manner as Intermediate 164 using Intermediate 390A instead of Intermediate 163.

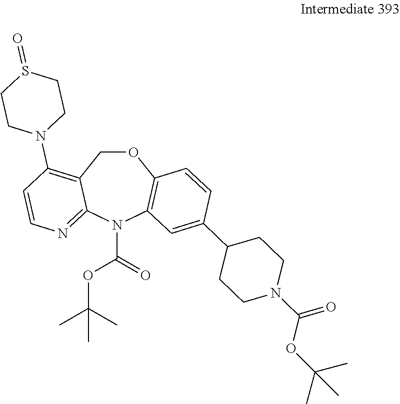 [SMILES: CC(C)(C)OC(=O)N1CCC(c2ccc3c(c2)N(C(=O)OC(C)(C)C)c2nccc(N4CCS(=O)CC4)c2CO3)CC1]

Intermediate 393 was synthesized in a similar manner as Intermediate 35 using thiomorpholine-1-oxide HCl [CAS: 76176-87-9] instead of morpholine and Intermediate 11 instead of Intermediate 34.

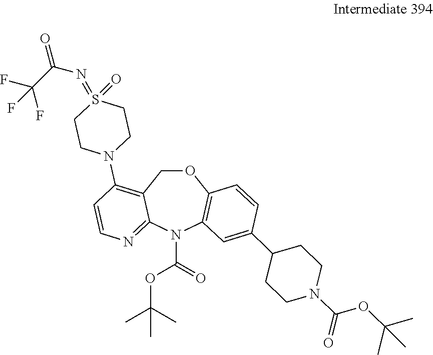 [SMILES: CC(C)(C)OC(=O)N1CCC(c2ccc3c(c2)N(C(=O)OC(C)(C)C)c2nccc(N4CCS(=O)(=NC(=O)C(F)(F)F)CC4)c2CO3)CC1]

(Diacetoxyiodo)benzene ([CAS: 3240-34-4], 1.4 g, 4.2 mmol, 1.5 eq.) was added to a solution of Intermediate 393 (1.7 g, 2.8 mmol, 1.0 eq.), trifluoroacetamide ([CAS: 354-38-1], 481 mg, 4.2 mmol, 1.5 eq.), magnesium oxide (458 mg, 11.3 mmol, 4.0 eq.), and dirhodium tetraacetate ([CAS: 15956-28-2], 63 mg, 0.3 mmol, 0.1 eq.) in DCM (30 mL) at room temperature. The reaction mixture was stirred at room temperature overnight. Water and DCM were added. The organic layer was separated, dried over MgSO₄, filtered, and evaporated. The residue was purified by column chromatography (Stationary phase: irregular SiOH 15-40 μm 25 g, Mobile phase: 98/2 DCM/MeOH to 95/5/0.1 DCM/MeOH/NH₃) yielding Intermediate 394 (1.33 g, yield: 66%).

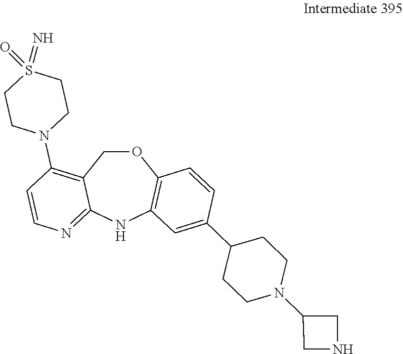 [SMILES: N=S1(=O)CCN(c2ccnc3c2COc2ccc(C4CCN(C5CNC5)CC4)cc2N3)CC1]

Intermediate 395 was synthesized following the synthetic route from Intermediate 162 to Intermediate 164 starting with Intermediate 394 instead of Intermediate 161.

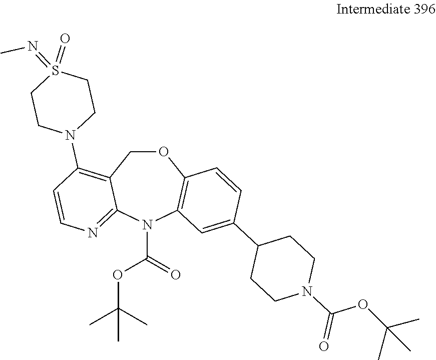 [SMILES: CN=S1(=O)CCN(c2ccnc3c2COc2ccc(C4CCN(C(=O)OC(C)(C)C)CC4)cc2N3C(=O)OC(C)(C)C)CC1]

Intermediate 396 was synthesized in a similar manner as Intermediate 35 using N-(1-oxido-1λ⁴-thiomorpholin-1-ylidene)methanamine [CAS: 1621962-34-2] instead of morpholine and Intermediate 11 instead of Intermediate 34.

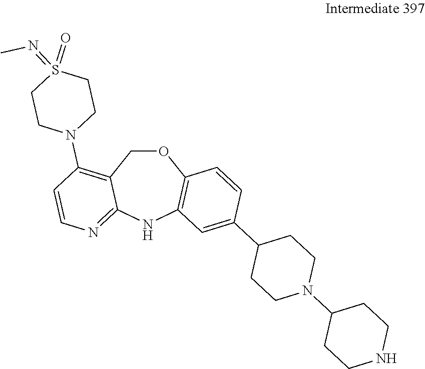 [SMILES: CN=S1(=O)CCN(c2ccnc3c2COc2ccc(C4CCN(C5CCNCC5)CC4)cc2N3)CC1]

Intermediate 397 was synthesized following the synthetic route from Intermediate 162 to Intermediate 164 starting with Intermediate 396 instead of Intermediate 161 and N-Boc-piperidin-4-one instead of N-Boc-3-azetidinone.

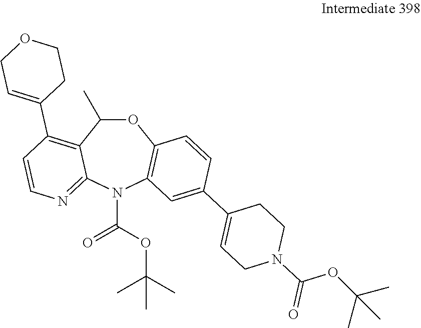 [SMILES: CC1Oc2ccc(C3=CCN(C(=O)OC(C)(C)C)CC3)cc2N(C(=O)OC(C)(C)C)c2nccc(C3=CCOCC3)c21]

Intermediate 398 was synthesized in a similar manner as Intermediate 356 using Intermediate 202 instead of Intermediate 355 and dihydropyran-4-boronic acid pinacol ester [CAS: 287944-16-5] instead of dihydrothiopyran-4-boronic acid pinacol ester.

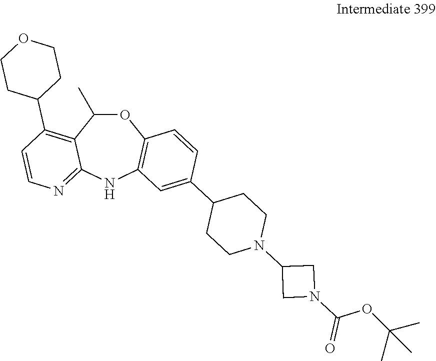 [SMILES: CC1Oc2ccc(C3CCN(C4CN(C(=O)OC(C)(C)C)C4)CC3)cc2Nc2nccc(C3CCOCC3)c21]

Intermediate 399 was synthesized following the synthetic route from Intermediate 22 to Intermediate 24 using Intermediate 398 instead of Intermediate 21.

### Intermediate 400A and Intermediate 400B

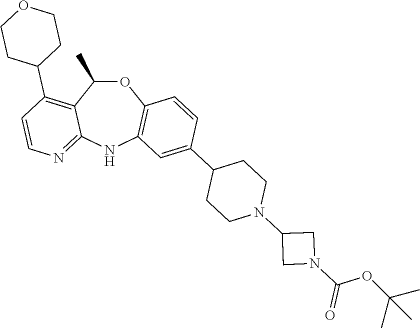 [SMILES: C[C@H]1Oc2ccc(C3CCN(C4CN(C(=O)OC(C)(C)C)C4)CC3)cc2Nc2nccc(C3CCOCC3)c21]

Intermediate 400A: (*R), pure stereoisomer but absolute stereochemistry undetermined

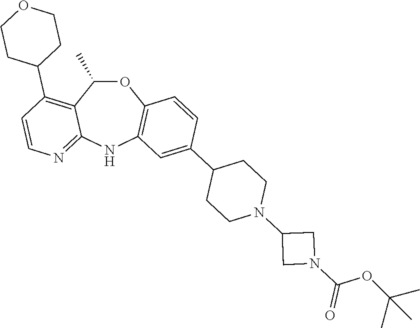 [SMILES: C[C@@H]1Oc2ccc(C3CCN(C4CN(C(=O)OC(C)(C)C)C4)CC3)cc2Nc2nccc(C3CCOCC3)c21]

Intermediate 400B: (*S), pure stereoisomer but absolute stereochemistry undetermined Intermediate 399 was separated into its enantiomers by chiral HPLC, (Method Cellulose-1 Q-MG3) to afford Intermediate 400A (860 mg, yield: 37%) and Intermediate 400B (340 mg, yield: 40%).

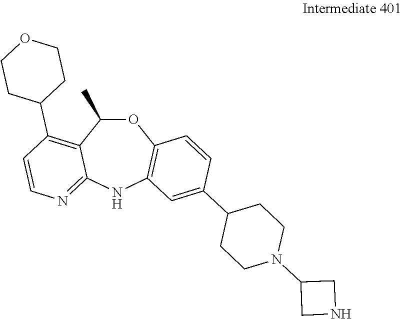 [SMILES: C[C@H]1Oc2ccc(C3CCN(C4CNC4)CC3)cc2Nc2nccc(C3CCOCC3)c21]

(*R), pure stereoisomer but absolute stereochemistry undetermined Intermediate 401 was synthesized in a similar manner as Intermediate 25 using Intermediate 400A instead of Intermediate 24.

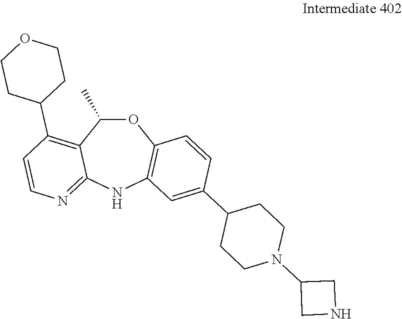 [SMILES: C[C@@H]1Oc2ccc(C3CCN(C4CNC4)CC3)cc2Nc2nccc(C3CCOCC3)c21]

(*S), pure stereoisomer but absolute stereochemistry undetermined Intermediate 402 was synthesized in a similar manner as Intermediate 25 using Intermediate 400B instead of Intermediate 24.

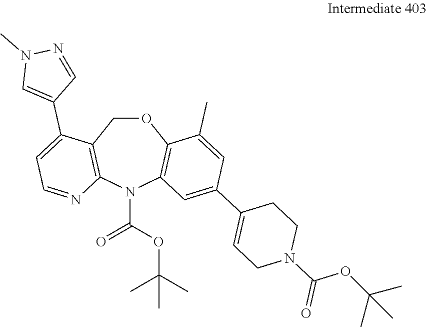 [SMILES: Cc1cc(C2=CCN(C(=O)OC(C)(C)C)CC2)cc2c1OCc1c(-c3cnn(C)c3)ccnc1N2C(=O)OC(C)(C)C]

Intermediate 403 was synthesized in a similar manner as Intermediate 6 using 1-methyl-4-(4,4,5,5-tetramethyl-1,3,2-dioxaborolan-2-yl)-1H-pyrazole [CAS: 761446-44-0] instead of N-Boc-1,2,3,6-tetrahydropyridine-4-boronic acid pinacol ester and Intermediate 20 instead of Intermediate 5.

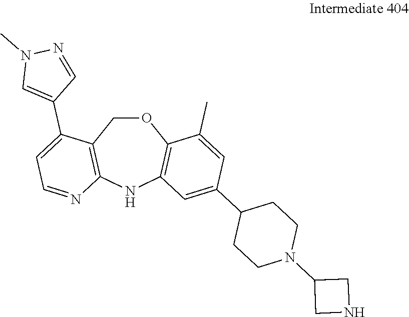 [SMILES: Cc1cc(C2CCN(C3CNC3)CC2)cc2c1OCc1c(-c3cnn(C)c3)ccnc1N2]

Intermediate 404 was synthesized following the synthetic route of Intermediate 22 to Intermediate 25 starting with Intermediate 403 instead of Intermediate 21.

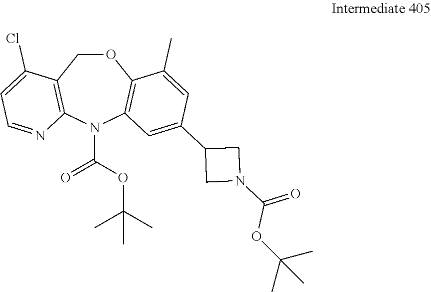 [SMILES: Cc1cc(C2CN(C(=O)OC(C)(C)C)C2)cc2c1OCc1c(Cl)ccnc1N2C(=O)OC(C)(C)C]

Intermediate 405 was synthesized in a similar manner as Intermediate 301 using Intermediate 19 instead of Intermediate 300.

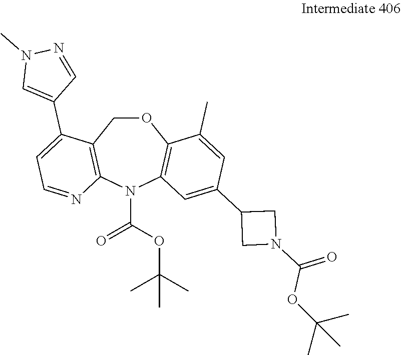 [SMILES: Cc1cc(C2CN(C(=O)OC(C)(C)C)C2)cc2c1OCc1c(-c3cnn(C)c3)ccnc1N2C(=O)OC(C)(C)C]

Intermediate 406 was synthesized in a similar manner as Intermediate 43 using Intermediate 405 instead of 2-chloro-4-iodo-3-pyridinecarboxaldehyde.

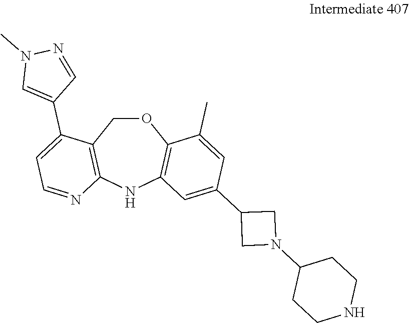 [SMILES: Cc1cc(C2CN(C3CCNCC3)C2)cc2c1OCc1c(-c3cnn(C)c3)ccnc1N2]

Intermediate 407 was synthesized following the synthetic route from Intermediate 23 to Intermediate 25 starting with Intermediate 406 instead of Intermediate 22 and using tert-butyl 4-oxopiperidine-1-carboxylate [CAS: 79099-07-3] instead of N-Boc-3-oxoazetidine.

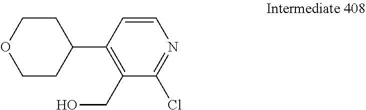 [SMILES: OCc1c(C2CCOCC2)ccnc1Cl]

Intermediate 408 was synthesized in a similar manner as Intermediate 309 starting with Intermediate 359 instead of Intermediate 308.

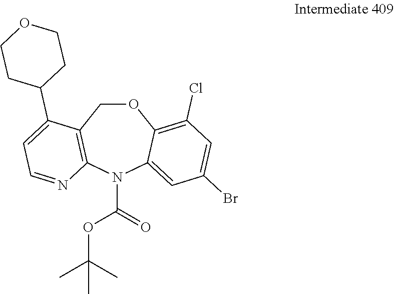 [SMILES: CC(C)(C)OC(=O)N1c2cc(Br)cc(Cl)c2OCc2c(C3CCOCC3)ccnc21]

Intermediate 409 was synthesized following the synthetic route from Intermediate 16 to Intermediate 19 starting with Intermediate 408 instead of 2,4-dichloro-3-pyridinemethanol and 4-bromo-2-chloro-6-nitrophenol [CAS: 58349-01-2] instead of 4-bromo-2-methyl-6-nitrophenol.

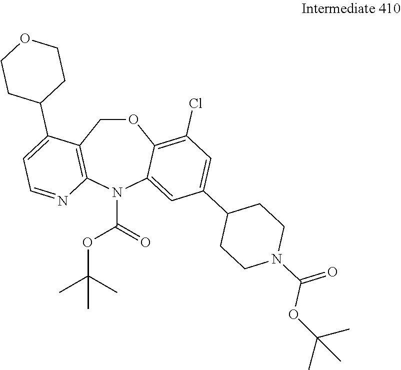 [SMILES: CC(C)(C)OC(=O)N1CCC(c2cc(Cl)c3c(c2)N(C(=O)OC(C)(C)C)c2nccc(C4CCOCC4)c2CO3)CC1]

Intermediate 410 was synthesized in a similar manner as Intermediate 211 using Intermediate 409 instead of Intermediate 210.

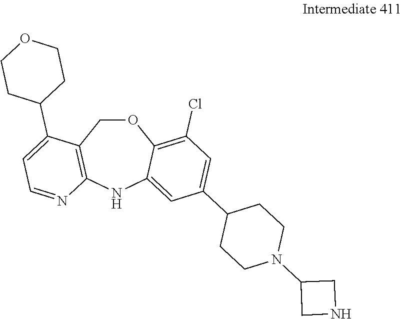 [SMILES: Clc1cc(C2CCN(C3CNC3)CC2)cc2c1OCc1c(C3CCOCC3)ccnc1N2]

Intermediate 411 was synthesized following the synthetic route of Intermediate 23 to Intermediate 25 starting with Intermediate 410 instead of Intermediate 22.

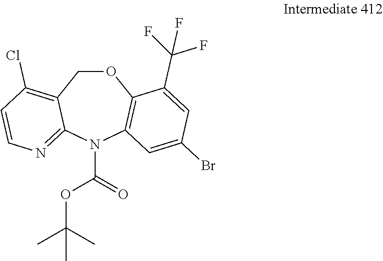 [SMILES: CC(C)(C)OC(=O)N1c2cc(Br)cc(C(F)(F)F)c2OCc2c(Cl)ccnc21]

Intermediate 412 was synthesized following the synthetic route from Intermediate 16 to Intermediate 19 starting with 4-bromo-2-nitro-6-(trifluoromethyl)phenol [CAS: 2089255-50-3] instead of 4-bromo-2-methyl-6-nitrophenol.

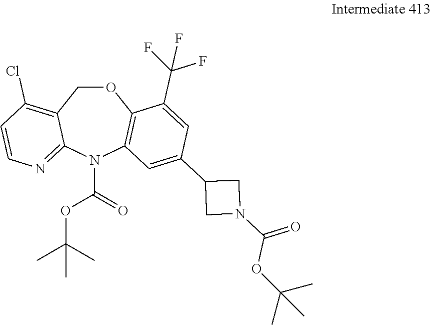 [SMILES: CC(C)(C)OC(=O)N1CC(c2cc3c(c(C(F)(F)F)c2)OCc2c(Cl)ccnc2N3C(=O)OC(C)(C)C)C1]

Intermediate 413 was synthesized in a similar manner as Intermediate 211 using Intermediate 412 instead of Intermediate 210 and [1-[(1,1-dimethylethoxy)carbonyl]-3-azetidinyl]iodozine [CAS: 206446-38-0] instead of [1-[(1,1-dimethylethoxy)carbonyl]-4-5_piperidinyl]iodozine.

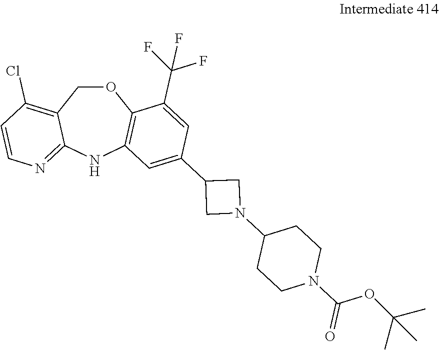 [SMILES: CC(C)(C)OC(=O)N1CCC(N2CC(c3cc4c(c(C(F)(F)F)c3)OCc3c(Cl)ccnc3N4)C2)CC1]

Intermediate 414 was synthesized following the synthetic route from Intermediate 23 to Intermediate 24 starting with Intermediate 413 instead of Intermediate 22 and using tert-butyl-4-oxopiperidine-1-carboxylate [CAS: 79099-07-3] instead of N-Boc-3-oxoazetidine.

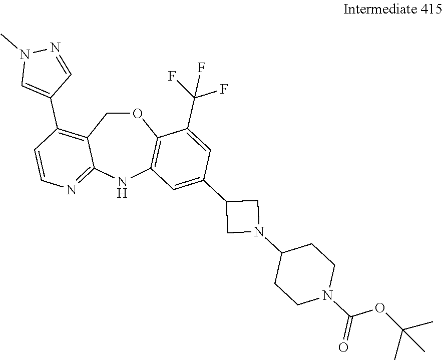 [SMILES: Cn1cc(-c2ccnc3c2COc2c(cc(C4CN(C5CCN(C(=O)OC(C)(C)C)CC5)C4)cc2C(F)(F)F)N3)cn1]

Intermediate 415 was synthesized in a similar manner as Intermediate 43 using Intermediate 414 instead of 2-chloro-4-iodo-3-pyridinecarboxaldehyde.

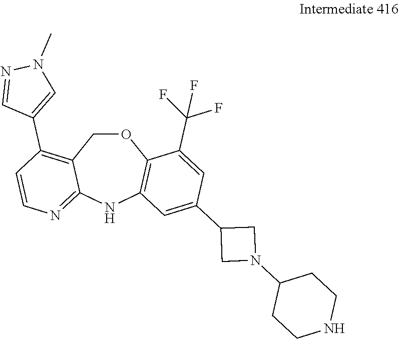 [SMILES: Cn1cc(-c2ccnc3c2COc2c(cc(C4CN(C5CCNCC5)C4)cc2C(F)(F)F)N3)cn1]

Intermediate 416 was synthesized in a similar manner as Intermediate 183 using Intermediate 415 instead of Intermediate 182.

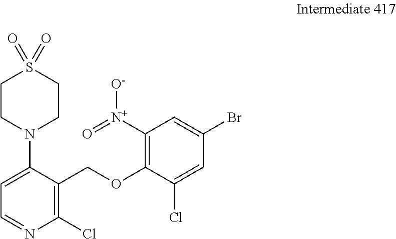 [SMILES: O=[N+]([O-])c1cc(Br)cc(Cl)c1OCc1c(N2CCS(=O)(=O)CC2)ccnc1Cl]

Intermediate 417 was synthesized in a similar manner as Intermediate 46 using Intermediate 310 instead of Intermediate 45 and 4-bromo-2-chloro-6-nitrophenol [CAS: 58349-01-2] instead of Intermediate 42.

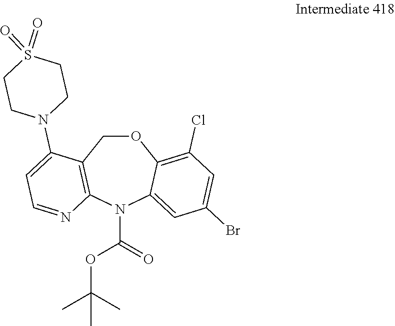 [SMILES: CC(C)(C)OC(=O)N1c2cc(Br)cc(Cl)c2OCc2c(N3CCS(=O)(=O)CC3)ccnc21]

Intermediate 418 was synthesized following the synthetic route from Intermediate 17 to Intermediate 19 starting with Intermediate 417 instead of Intermediate 16.

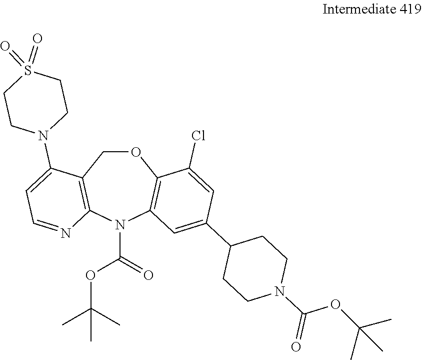 [SMILES: CC(C)(C)OC(=O)N1CCC(c2cc(Cl)c3c(c2)N(C(=O)OC(C)(C)C)c2nccc(N4CCS(=O)(=O)CC4)c2CO3)CC1]

Intermediate 419 was synthesized in a similar manner as Intermediate 211 using Intermediate 418 instead of Intermediate 210.

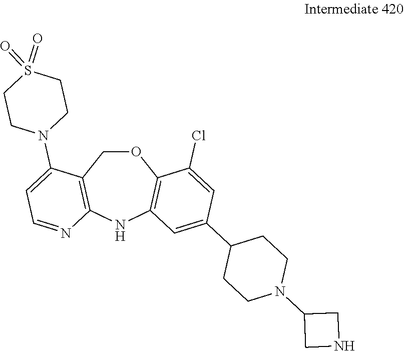 [SMILES: O=S1(=O)CCN(c2ccnc3c2COc2c(Cl)cc(C4CCN(C5CNC5)CC4)cc2N3)CC1]

Intermediate 420 was synthesized following the synthetic route of Intermediate 23 to Intermediate 25 starting with Intermediate 419 instead of Intermediate 22.

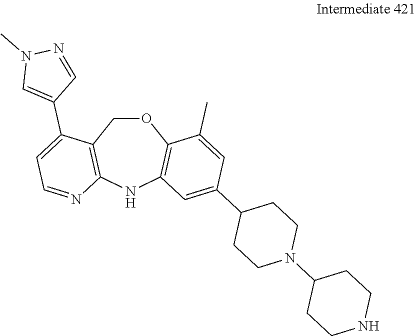 [SMILES: Cc1cc(C2CCN(C3CCNCC3)CC2)cc2c1OCc1c(-c3cnn(C)c3)ccnc1N2]

Intermediate 421 was synthesized following the synthetic route of Intermediate 22 to Intermediate 25 starting with Intermediate 403 instead of Intermediate 21 and using tert-butyl 4-oxopiperidine-1-carboxylate [CAS: 79099-07-3] instead of N-Boc-3-oxoazetidine.

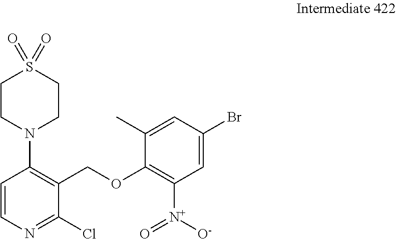 [SMILES: Cc1cc(Br)cc([N+](=O)[O-])c1OCc1c(N2CCS(=O)(=O)CC2)ccnc1Cl]

Intermediate 422 was synthesized in a similar manner as Intermediate 311 using 4-bromo-2-methyl-6-nitrophenol [CAS: 20294-50-2] instead of 4-iodo-2-nitrophenol.

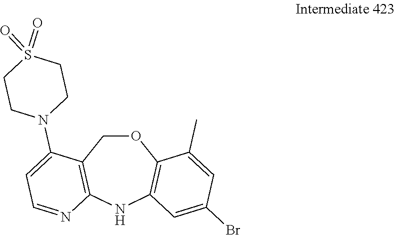 [SMILES: Cc1cc(Br)cc2c1OCc1c(N3CCS(=O)(=O)CC3)ccnc1N2]

Intermediate 423 was synthesized in a similar manner as Intermediate 192 using Intermediate 422 instead of Intermediate 191.

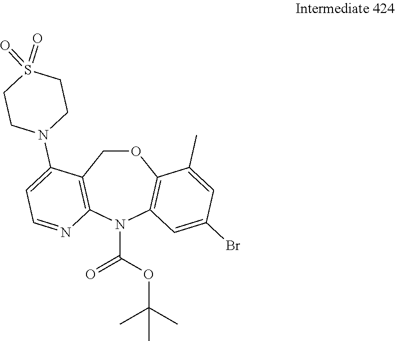 [SMILES: Cc1cc(Br)cc2c1OCc1c(N3CCS(=O)(=O)CC3)ccnc1N2C(=O)OC(C)(C)C]

Intermediate 424 was synthesized in a similar manner as Intermediate 202 using Intermediate 423 instead of Intermediate 201.

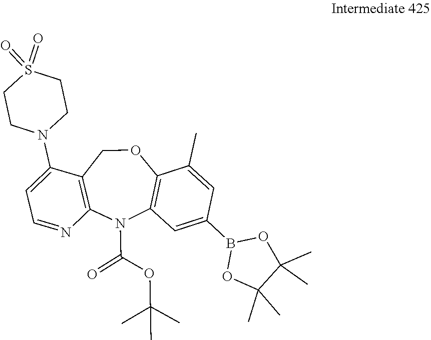 [SMILES: Cc1cc(B2OC(C)(C)C(C)(C)O2)cc2c1OCc1c(N3CCS(=O)(=O)CC3)ccnc1N2C(=O)OC(C)(C)C]

Intermediate 425 was synthesized in a similar manner as Intermediate 267 using Intermediate 424 instead of Intermediate 175.

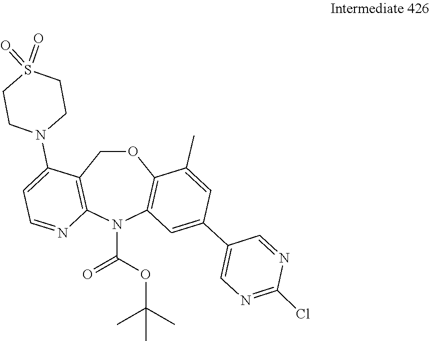 [SMILES: Cc1cc(-c2cnc(Cl)nc2)cc2c1OCc1c(N3CCS(=O)(=O)CC3)ccnc1N2C(=O)OC(C)(C)C]

Intermediate 426 was synthesized in a similar manner as Intermediate 290 using Intermediate 425 instead of Intermediate 267 and 5-bromo-2-chloropyrimidine [CAS. 32779-36-5] instead of 5-bromo-2-chloro-4,6-dimethylpyrimidine.

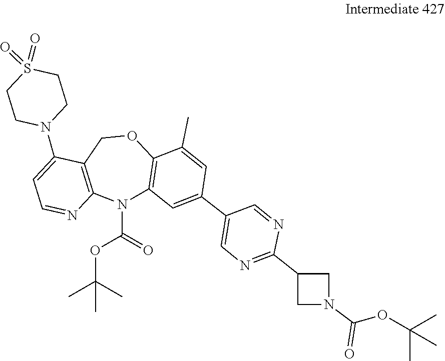 [SMILES: Cc1cc(-c2cnc(C3CN(C(=O)OC(C)(C)C)C3)nc2)cc2c1OCc1c(N3CCS(=O)(=O)CC3)ccnc1N2C(=O)OC(C)(C)C]

Intermediate 427 was synthesized in a similar manner as Intermediate 301 using Intermediate 426 instead of Intermediate 300.

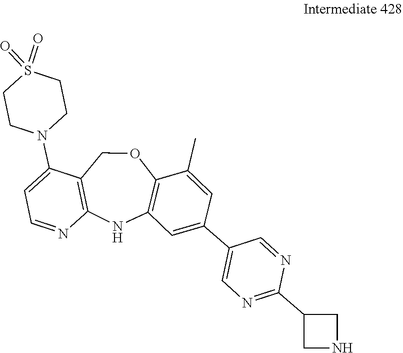 [SMILES: Cc1cc(-c2cnc(C3CNC3)nc2)cc2c1OCc1c(N3CCS(=O)(=O)CC3)ccnc1N2]

Intermediate 428 was synthesized in a similar manner as Intermediate 124 using Intermediate 427 instead of Intermediate 123.

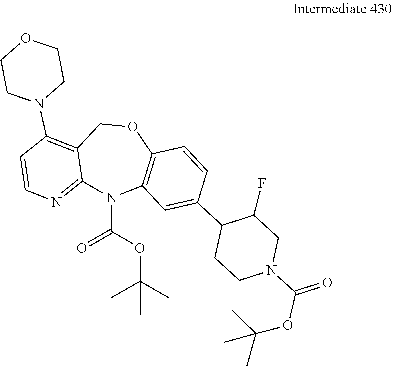 [SMILES: CC(C)(C)OC(=O)N1CCC(c2ccc3c(c2)N(C(=O)OC(C)(C)C)c2nccc(N4CCOCC4)c2CO3)C(F)C1]

(Diethylamino)sulfur trifluoride ([CAS: 38078-09-0], 0.5 mL, 3.4 mmol, 2.0 eq.) was added to a mixture of Intermediate 340 (1 g, 1.7 mmol) in DCM at 0° C. under nitrogen atmosphere. The reaction mixture was stirred at room temperature for 2 h. Saturated aqueous NaHCO₃ was added and the organic layers were separated, dried over MgSO₄, filtered, and concentrated to dryness to yield Intermediate 430 (1 g, yield: 100%) as a foam.

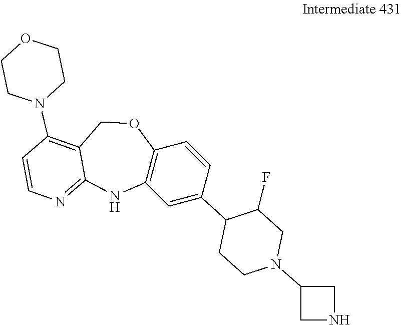 [SMILES: FC1CN(C2CNC2)CCC1c1ccc2c(c1)Nc1nccc(N3CCOCC3)c1CO2]

Intermediate 431 was synthesized following the synthetic route of Intermediate 23 to Intermediate 25 starting with Intermediate 430 instead of Intermediate 22 and using tert-butyl 4-oxopiperidine-1-carboxylate [CAS: 79099-07-3] instead of N-Boc-3-oxoazetidine.

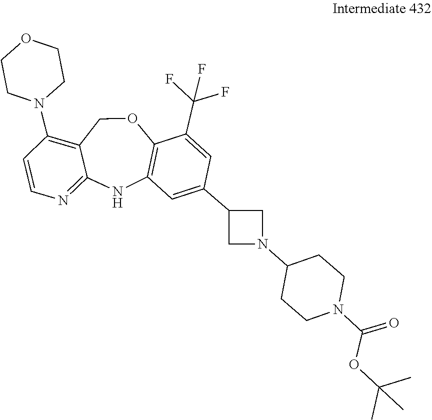 [SMILES: CC(C)(C)OC(=O)N1CCC(N2CC(c3cc4c(c(C(F)(F)F)c3)OCc3c(N5CCOCC5)ccnc3N4)C2)CC1]

Intermediate 432 was synthesized in a similar manner as Intermediate 12 using Intermediate 414 instead of Intermediate 11.

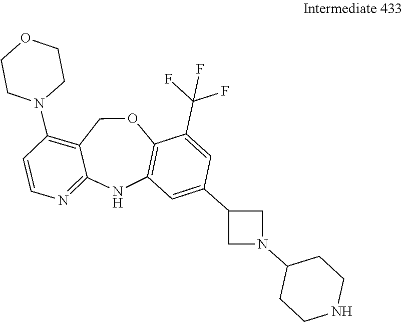 [SMILES: FC(F)(F)c1cc(C2CN(C3CCNCC3)C2)cc2c1OCc1c(N3CCOCC3)ccnc1N2]

Intermediate 433 was synthesized in a similar manner as Intermediate 183 using Intermediate 432 instead of Intermediate 182.

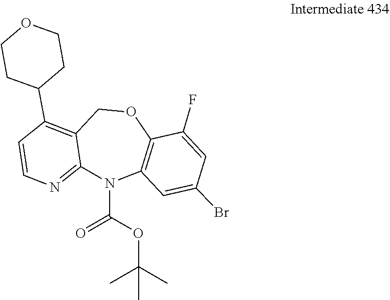 [SMILES: CC(C)(C)OC(=O)N1c2cc(Br)cc(F)c2OCc2c(C3CCOCC3)ccnc21]

Intermediate 434 was synthesized following the synthetic route from Intermediate 16 to Intermediate 19 starting Intermediate 408 instead of 2,4-dichloro-3-pyridinemethanol [CAS: 945543-24-8] and 4-bromo-2-fluoro-6-nitrophenol [CAS: 320-76-3] instead of 4-bromo-2-methyl-6-nitrophenol.

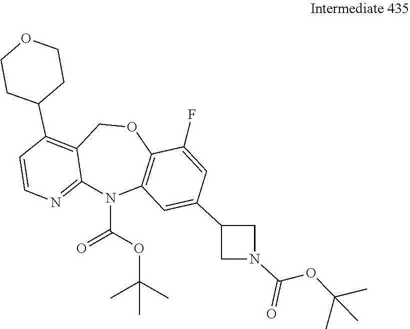 [SMILES: CC(C)(C)OC(=O)N1CC(c2cc(F)c3c(c2)N(C(=O)OC(C)(C)C)c2nccc(C4CCOCC4)c2CO3)C1]

Intermediate 435 was synthesized in a similar manner as Intermediate 301 using Intermediate 434 instead of Intermediate 300.

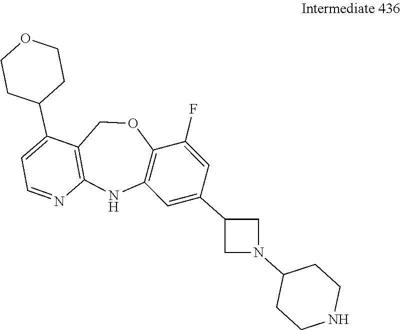 [SMILES: Fc1cc(C2CN(C3CCNCC3)C2)cc2c1OCc1c(C3CCOCC3)ccnc1N2]

Intermediate 436 was synthesized following the synthetic route of Intermediate 23 to Intermediate 25 starting with Intermediate 435 instead of Intermediate 22 and using tert-butyl 4-oxopiperidine-1-carboxylate [CAS: 79099-07-3] instead of N-Boc-3-oxoazetidine.

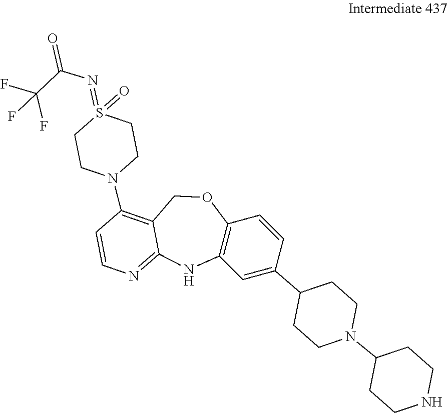 [SMILES: O=C(N=S1(=O)CCN(c2ccnc3c2COc2ccc(C4CCN(C5CCNCC5)CC4)cc2N3)CC1)C(F)(F)F]

Intermediate 437 was synthesized following the synthetic route of Intermediate 23 to Intermediate 25 starting with Intermediate 394 instead of Intermediate 22 and using tert-butyl 4-oxopiperidine-1-carboxylate [CAS: 79099-07-3] instead of N-Boc-3-oxoazetidine.

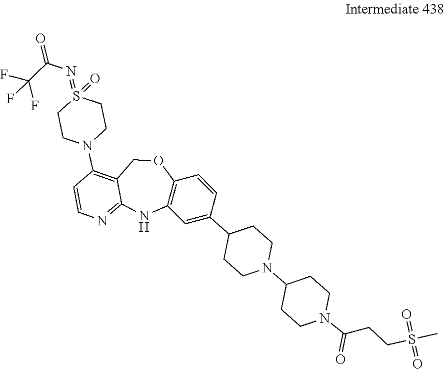 [SMILES: CS(=O)(=O)CCC(=O)N1CCC(N2CCC(c3ccc4c(c3)Nc3nccc(N5CCS(=O)(=NC(=O)C(F)(F)F)CC5)c3CO4)CC2)CC1]

A mixture of Intermediate 437 (284 mg, 0.4 mmol) and Et₃N (0.7 mL, 5.2 mmol) in DMF (5 mL) was stirred at room temperature for 10 min. EDCI·HCl (CAS [25952-53-8], 231 mg, 1.2 mmol) and 3-(methylsulfonyl)propanoic acid (CAS [645-83-0], 306 mg, 2 mmol) were added and the reaction mixture was stirred at room temperature overnight. Water and EtOAc were added. The layers were separated. The organic layer was separated, dried over MgSO₄, filtered, and evaporated. The residue was cristallized in ACN, yielding a first batch of Intermediate 438 (73 mg, yield: 25%). The filtrate was evaporated and the residue was cristallized in ACN, yielding a second batch of Intermediate 438 (40 mg, yield: 14%).

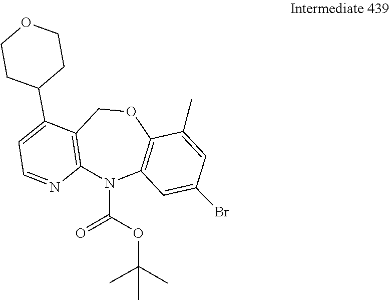 [SMILES: Cc1cc(Br)cc2c1OCc1c(C3CCOCC3)ccnc1N2C(=O)OC(C)(C)C]

Intermediate 439 was synthesized following the synthetic route from Intermediate 16 to Intermediate 19 starting from Intermediate 408 instead of 2,4-dichloro-3-pyridinemethanol.

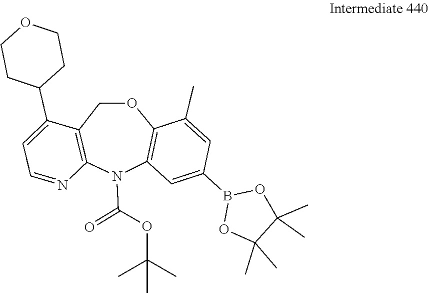 [SMILES: Cc1cc(B2OC(C)(C)C(C)(C)O2)cc2c1OCc1c(C3CCOCC3)ccnc1N2C(=O)OC(C)(C)C]

Intermediate 440 was synthesized in a similar manner as Intermediate 267 using Intermediate 439 instead of Intermediate 175.

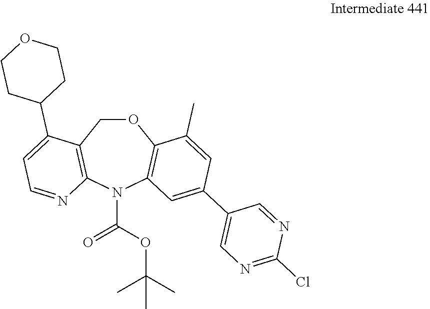 [SMILES: Cc1cc(-c2cnc(Cl)nc2)cc2c1OCc1c(C3CCOCC3)ccnc1N2C(=O)OC(C)(C)C]

Intermediate 441 was synthesized in a similar manner as Intermediate 290 using Intermediate 440 instead of Intermediate 267 and 5-bromo-2-chloropyrimidine [CAS. 32779-36-5] instead of 5-bromo-2-chloro-4,6-dimethylpyrimidine.

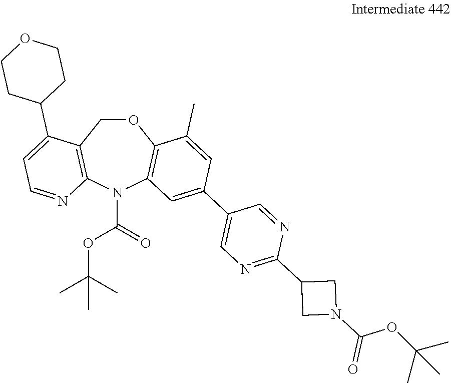 [SMILES: Cc1cc(-c2cnc(C3CN(C(=O)OC(C)(C)C)C3)nc2)cc2c1OCc1c(C3CCOCC3)ccnc1N2C(=O)OC(C)(C)C]

Intermediate 442 was synthesized in a similar manner as Intermediate 301 using Intermediate 441 instead of Intermediate 300.

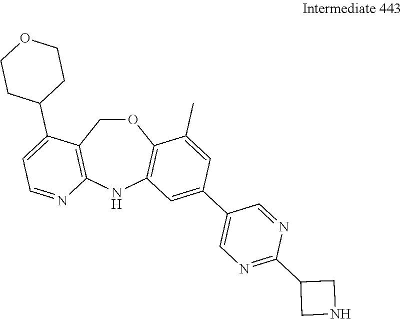 [SMILES: Cc1cc(-c2cnc(C3CNC3)nc2)cc2c1OCc1c(C3CCOCC3)ccnc1N2]

Intermediate 443 was synthesized in a similar manner as Intermediate 222 using Intermediate 442 instead of Intermediate 221.

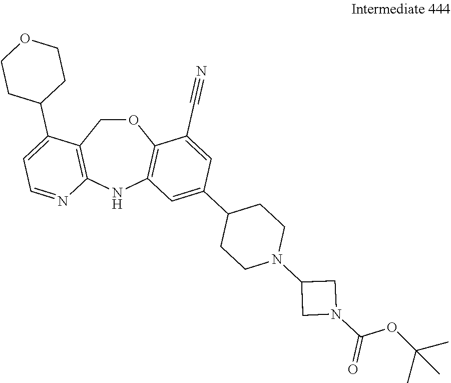 [SMILES: CC(C)(C)OC(=O)N1CC(N2CCC(c3cc(C#N)c4c(c3)Nc3nccc(C5CCOCC5)c3CO4)CC2)C1]

Zinc cyanide (72 mg, 0.61 mmol, 0.6 eq.), Pd(dppf)Cl₂ DCM (46 mg, 0.051 mmol, 0.05 eq.), and zinc powder (1.3 mg, 0.02 mmol, 0.02 eq.) were added to a solution of Intermediate 375 (609 mg, 1.02 mmol, 1.0 eq.) in DMA (15 mL) under nitrogen atmosphere. The reaction mixture was purged with nitrogen for 15 min and stirred at 100° C. for 2 h. The reaction mixture was allowed to cool to room temperature and was diluted with water (100 mL) and EtOAc (50 mL). The organic layer was separated, washed with brine, dried over MgSO₄, filtered, and concentrated. The residue was purified by column chromatography over silica gel (gradient of EtOAc in heptane from 25 to 100%) to give Intermediate 444 (496 mg, yield: 86%) as a beige solid.

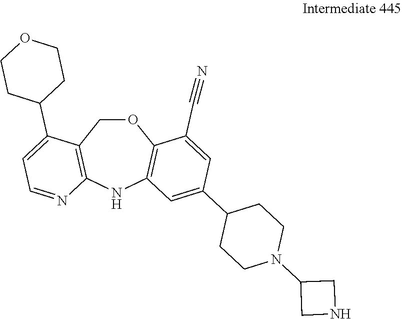 [SMILES: N#Cc1cc(C2CCN(C3CNC3)CC2)cc2c1OCc1c(C3CCOCC3)ccnc1N2]

Intermediate 445 was synthesized in a similar manner as Intermediate 8 using Intermediate 444 instead of Intermediate 7.

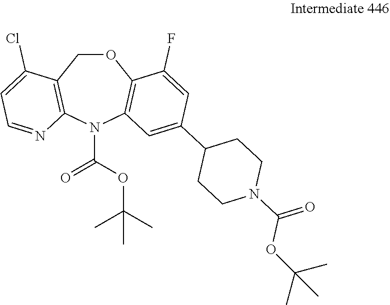 [SMILES: CC(C)(C)OC(=O)N1CCC(c2cc(F)c3c(c2)N(C(=O)OC(C)(C)C)c2nccc(Cl)c2CO3)CC1]

Intermediate 446 was synthesized in a similar manner as Intermediate 301 using Intermediate 321 instead of Intermediate 300 and [1-[(1,1-dimethylethoxy)carbonyl]-4-5piperidinyl]iodozine [CAS: 807618-13-9] instead of [1-[(1,1-dimethylethoxy)carbonyl]-3-azetidinyl]iodozine.

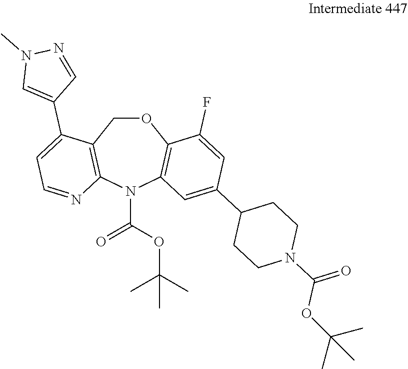 [SMILES: Cn1cc(-c2ccnc3c2COc2c(F)cc(C4CCN(C(=O)OC(C)(C)C)CC4)cc2N3C(=O)OC(C)(C)C)cn1]

Intermediate 447 was synthesized in a similar manner as Intermediate 368 using Intermediate 446 instead of Intermediate 4.

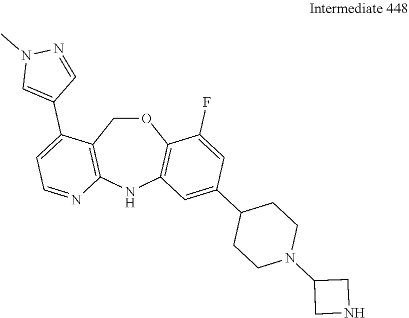 [SMILES: Cn1cc(-c2ccnc3c2COc2c(F)cc(C4CCN(C5CNC5)CC4)cc2N3)cn1]

Intermediate 448 was synthesized following the synthetic route from Intermediate 63 to Intermediate 65 starting with Intermediate 447 instead of Intermediate 62.

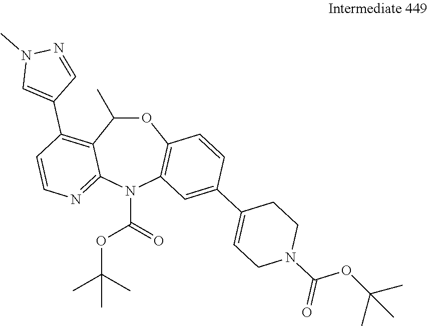 [SMILES: CC1Oc2ccc(C3=CCN(C(=O)OC(C)(C)C)CC3)cc2N(C(=O)OC(C)(C)C)c2nccc(-c3cnn(C)c3)c21]

Intermediate 449 was synthesized in a similar manner as Intermediate 368 using Intermediate 202 instead of Intermediate 367.

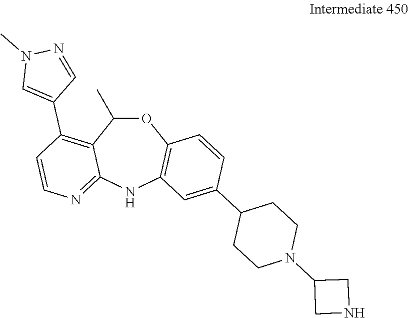 [SMILES: CC1Oc2ccc(C3CCN(C4CNC4)CC3)cc2Nc2nccc(-c3cnn(C)c3)c21]

Intermediate 450 was synthesized following the synthetic route from Intermediate 22 to Intermediate 25 starting with Intermediate 449 instead of Intermediate 21.

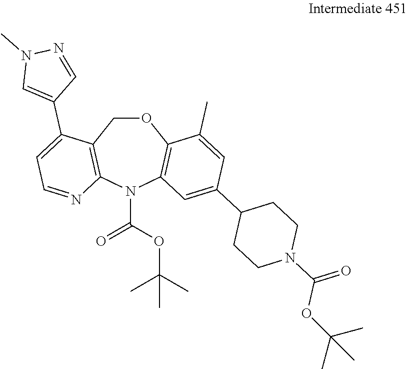 [SMILES: Cc1cc(C2CCN(C(=O)OC(C)(C)C)CC2)cc2c1OCc1c(-c3cnn(C)c3)ccnc1N2C(=O)OC(C)(C)C]

Intermediate 451 was synthesized in a similar manner as Intermediate 368 using Intermediate 355 instead of Intermediate 367.

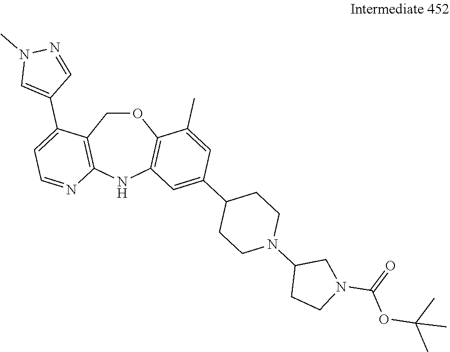 [SMILES: Cc1cc(C2CCN(C3CCN(C(=O)OC(C)(C)C)C3)CC2)cc2c1OCc1c(-c3cnn(C)c3)ccnc1N2]

Intermediate 452 was synthesized following the route from Intermediate 63 to Intermediate 64 starting with Intermediate 451 instead of Intermediate 62 and using 3-oxopyrrolidine-1-carboxylic acid tert-butyl ester [CAS: 101385-93-7] instead of tert-butyl 3-oxoazetidine-1-carboxylate.

### Intermediate 453A and Intermediate 453B

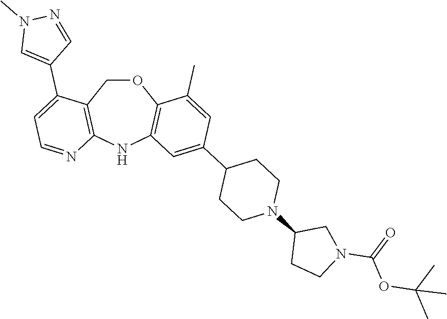 [SMILES: Cc1cc(C2CCN([C@@H]3CCN(C(=O)OC(C)(C)C)C3)CC2)cc2c1OCc1c(-c3cnn(C)c3)ccnc1N2]

Intermediate 453A: (*R), pure stereoisomer but absolute stereochemistry undetermined

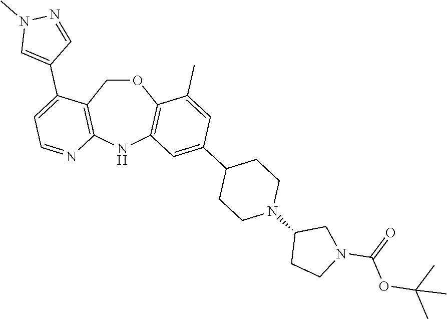 [SMILES: Cc1cc(C2CCN([C@H]3CCN(C(=O)OC(C)(C)C)C3)CC2)cc2c1OCc1c(-c3cnn(C)c3)ccnc1N2]

Intermediate 453B: (*S), pure stereoisomer but absolute stereochemistry undetermined Intermediate 452 was separated into its enantiomers by chiral phase column chromatography (Phenomenex Lux Amylose-1 150×21.2 mm 5 μm; gradient from 50% heptane with 0.1% DEA−50% iPrOH with 0.1% DEA to 100% iPrOH with 0.1% DEA) to give Intermediate 453A (391 mg, yield: 37%) as a clear oil and Intermediate 453B (390 mg, yield: 37%) as a clear oil.

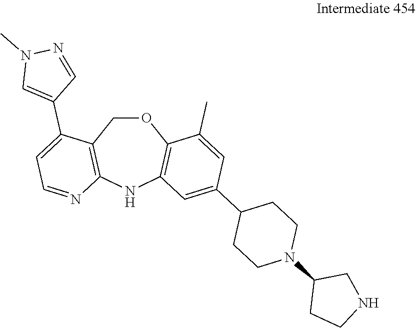 [SMILES: Cc1cc(C2CCN([C@@H]3CCNC3)CC2)cc2c1OCc1c(-c3cnn(C)c3)ccnc1N2]

(*R), pure stereoisomer but absolute stereochemistry undetermined Intermediate 454 was synthesized in a similar manner as Intermediate 146 using Intermediate 453A instead of Intermediate 145.

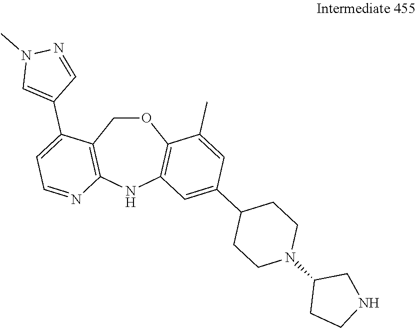 [SMILES: Cc1cc(C2CCN([C@H]3CCNC3)CC2)cc2c1OCc1c(-c3cnn(C)c3)ccnc1N2]

(*S), pure stereoisomer but absolute stereochemistry undetermined Intermediate 455 was synthesized in a similar manner as Intermediate 146 using Intermediate 453B instead of Intermediate 145.

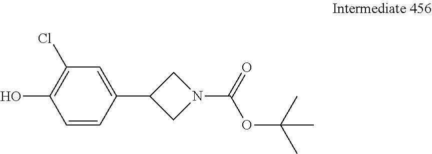 [SMILES: CC(C)(C)OC(=O)N1CC(c2ccc(O)c(Cl)c2)C1]

Intermediate 456 was synthesized in a similar manner as Intermediate 327 using tert-butyl 3-iodoazetidine-1-carboxylate [CAS: 254454-54-1] instead of Intermediate 326 and 4-bromo-2-chlorophenol [CAS: 3964-56-5] instead of Intermediate 231.

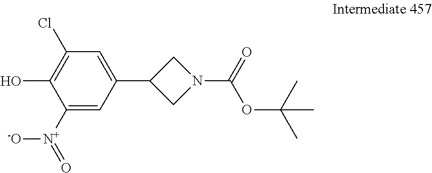

Intermediate 457 was synthesized in a similar manner as Intermediate 361 using Intermediate 456 instead of tert-butyl 4-(4-hydroxyphenyl)piperidine-1-carboxylate.

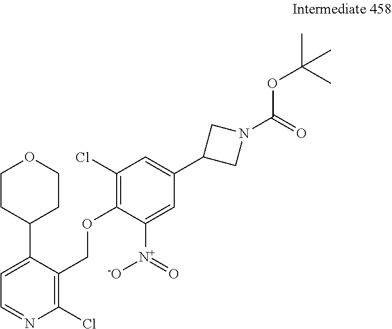

Intermediate 458 was synthesized in a similar manner as Intermediate 311 using Intermediate 360 instead of Intermediate 310 and Intermediate 457 instead of 4-iodo-2-nitrophenol.

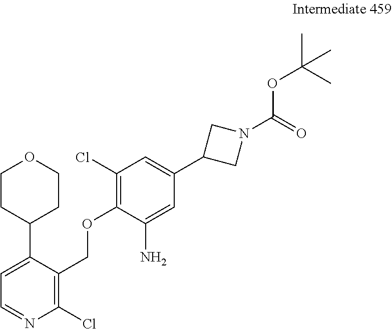 [SMILES: CC(C)(C)OC(=O)N1CC(c2cc(N)c(OCc3c(C4CCOCC4)ccnc3Cl)c(Cl)c2)C1]

Intermediate 459 was synthesized in a similar manner as Intermediate 2 using Intermediate 458 instead of Intermediate 1.

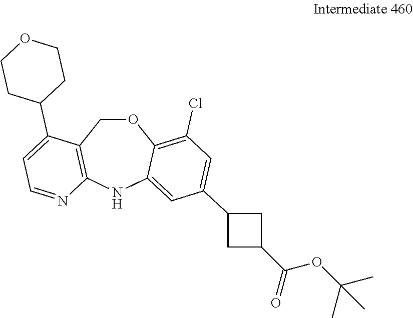 [SMILES: CC(C)(C)OC(=O)C1CC(c2cc(Cl)c3c(c2)Nc2nccc(C4CCOCC4)c2CO3)C1]

LiHMDS ([CAS: 4039-32-1], 1.0 M in THF, 5.2 mL, 5.2 mmol, 3.2 eq.) was added at 0° C. under nitrogen atmosphere to Intermediate 459 (825 mg, 1.6 mmol) in THF (4.3 mL) in a closed vial. The reaction mixture was stirred at room temperature for 7 h. The reaction mixture was diluted with HCl (1 M in water) and extracted with DCM. The organic layer was dried (Na₂SO₄), filtered, and the solvent was evaporated. The residue was purified by column chromatography (silica, MeOH in DCM 2/98 to 6/94), yielding Intermediate 460 (641 mg, yield: 53%) as a yellow oil.

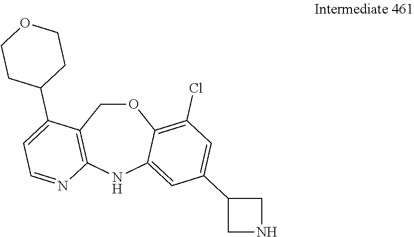 [SMILES: Clc1cc(C2CNC2)cc2c1OCc1c(C3CCOCC3)ccnc1N2]

HCl (4 M in dioxane, 6.0 mL, 23.9 mmol, 28.0 eq.) was added to Intermediate 460 (641 mg, 0.86 mmol) and the mixture was stirred for 2 h at 0° C. The solvent was evaporated, yielding Intermediate 461 (318 mg, yield: 71%) used without further purification.

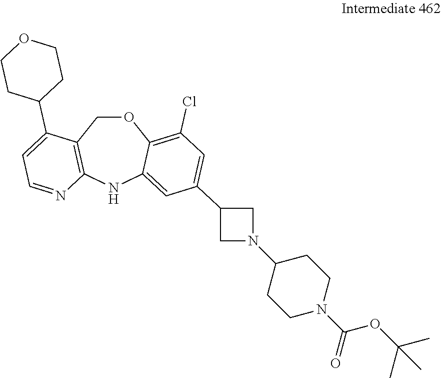 [SMILES: CC(C)(C)OC(=O)N1CCC(N2CC(c3cc(Cl)c4c(c3)Nc3nccc(C5CCOCC5)c3CO4)C2)CC1]

Et₃N (0.5 mL, 3.4 mmol, 4.0 eq.) was added to a mixture of Intermediate 461 (318 mg, 0.86 mmol) and tert-butyl 4-oxopiperidine-1-carboxylate ([CAS: 79099-07-3], 170 mg, 0.855 mmol, 1 eq.) in ACN (4 mL). NaBH(OAc)₃ (544 mg, 2.6 mmol, 3.0 eq.) was added and the reaction mixture was stirred for 1 h 45 min at room temperature. Then the reaction was diluted with water, basified with drops of 5% aqueous K₂CO₃ and extracted with DCM. The organic layer was separated, dried (Na₂SO₄), filtered, and the solvent evaporated. The residue was purified by column chromatography (silica, MeOH in DCM 2/98 to 10/90), yielding Intermediate 462 (497 mg, yield: 80%).

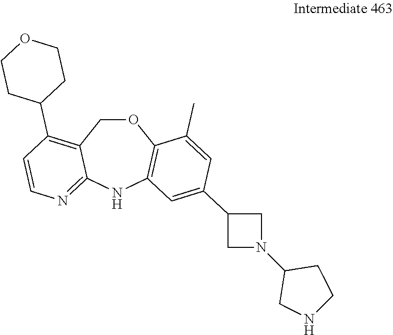 [SMILES: Cc1cc(C2CN(C3CCNC3)C2)cc2c1OCc1c(C3CCOCC3)ccnc1N2]

Intermediate 463 was synthesized following the synthetic route from Intermediate 12 to Intermediate 15 starting with Intermediate 405 instead of Intermediate 11 and using pyrrolidin-3-one, N-Boc protected [CAS: 101385-93-7] instead of 1-Boc-3-azetidinone.

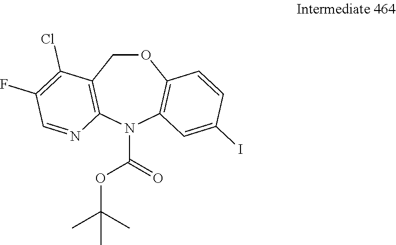 [SMILES: CC(C)(C)OC(=O)N1c2cc(I)ccc2OCc2c1ncc(F)c2Cl]

Intermediate 464 was synthesized following the synthetic route from Intermediate 16 to Intermediate 19 starting with 4-chloro-2,5-difluoro-3-pyridinemethanol [CAS 1260788-41-7] instead of 2,4-dichloro-3-pyridinemethanol and 4-iodo-2-nitrophenol [CAS 21784-73-6] instead of 4-bromo-2-methyl-6-nitrophenol.

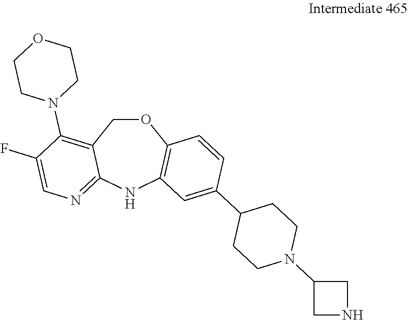 [SMILES: Fc1cnc2c(c1N1CCOCC1)COc1ccc(C3CCN(C4CNC4)CC3)cc1N2]

Intermediate 465 was synthesized following the synthetic route from Intermediate 4 to Intermediate 10 starting with Intermediate 464 instead of Intermediate 4.

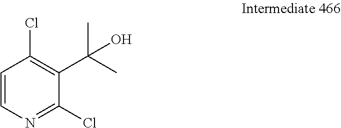

To a solution of diisopropyl amine (24.6 mL, 175.7 mmol, 1.3 eq.) in dry THF (100 mL) at 0° C. under nitrogen, nBuLi (2.5 M solution, 70.3 mL, 175.7 mmol, 1.3 eq.) was added and the mixture was stirred at 0° C. for 30 min. The resulting solution was added via syringe to a solution of 2,4-dichloropyridine ([CAS: 26452-80-2], 20 g, 135.1 mmol, 1.0 eq.) in dry THF (100 mL) at −78° C. under nitrogen atmosphere and the reaction mixture was stirred at −78° C. for 1 h. Acetone (29.8 mL, 405.4 mmol, 3.0 eq.) was then added dropwise over 30 min and the mixture was stirred for 1 h at −78° C. The reaction mixture was quenched with saturated aqueous NH₄Cl and extracted with EtOAc. The organic layer was washed with brine, dried (MgSO₄), and concentrated. Purification was performed by flash column chromatography (SiO₂, EtOAc-heptane) to yield Intermediate 466 (5.3 g, yield: 19%).

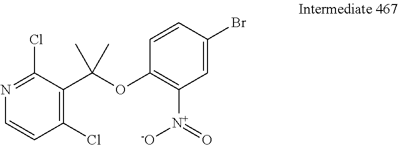

Sodium hexamethyldisilyl amide solution (1 M in THF, 27.4 mL, 27.4 mmol, 1.1 eq.) was added to a solution of Intermediate 466 (5.23 g, 24.9 mmol, 1.0 eq.) in THF (60 mL) at 0° C. under nitrogen atmosphere and the mixture was stirred for 5 min. 4-Bromo-1-fluoro-2-nitrobenzene ([CAS: 7693-52-9], 6.6 g, 29.9 mmol, 1.2 eq.) was added and the reaction mixture was stirred while warming to room temperature for 1 h. The reaction mixture was diluted with EtOAc and quenched with aqueous ammonium chloride. Layers were separated, the organic layer was dried with brine, dried (MgSO₄), and concentrated. Purification was performed by column flash chromatography (SiO₂, EtOAc-heptane gradient) to yield Intermediate 467 (1.3 g, yield: 13%).

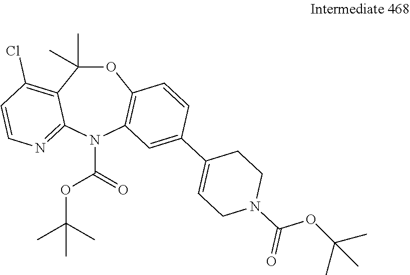 [SMILES: CC(C)(C)OC(=O)N1CC=C(c2ccc3c(c2)N(C(=O)OC(C)(C)C)c2nccc(Cl)c2C(C)(C)O3)CC1]

Intermediate 468 was synthesized following the synthetic route from Intermediate 17 to Intermediate 20 starting from Intermediate 467 instead of Intermediate 16.

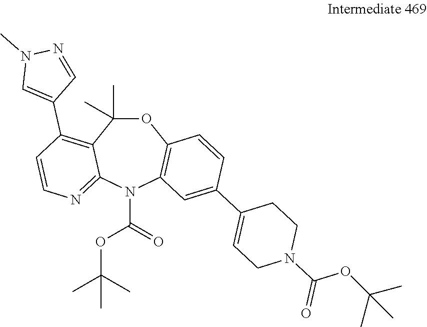 [SMILES: Cn1cc(-c2ccnc3c2C(C)(C)Oc2ccc(C4=CCN(C(=O)OC(C)(C)C)CC4)cc2N3C(=O)OC(C)(C)C)cn1]

Intermediate 469 was synthesized in a similar manner as Intermediate 368 using Intermediate 468 instead of Intermediate 367.

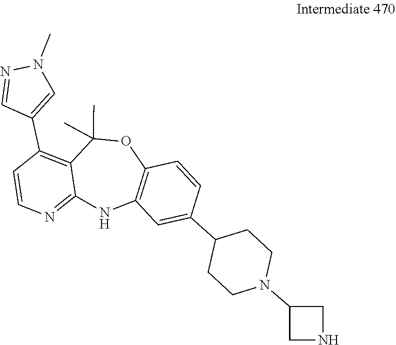 [SMILES: Cn1cc(-c2ccnc3c2C(C)(C)Oc2ccc(C4CCN(C5CNC5)CC4)cc2N3)cn1]

Intermediate 470 was synthesized following the synthetic route from Intermediate 22 to Intermediate 25 starting from Intermediate 469 instead of Intermediate 21.

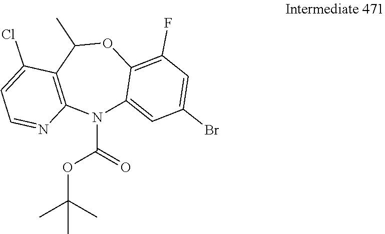 [SMILES: CC1Oc2c(F)cc(Br)cc2N(C(=O)OC(C)(C)C)c2nccc(Cl)c21]

Intermediate 471 was synthesized following the synthetic route from Intermediate 16 to Intermediate 19 starting with 2,4-dichloro-α-methyl-3-pyridinemethanol [CAS 1246349-88-1] instead of 2,4-dichloro-3-pyridinemethanol and 4-bromo-2-fluoro-6-nitrophenol [CAS: 320-76-3] instead of 4-bromo-2-methyl-6-nitrophenol.

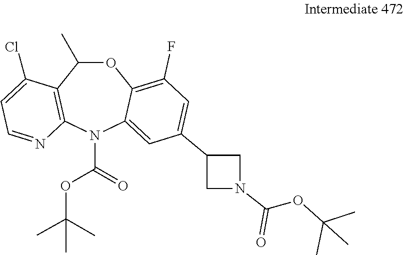 [SMILES: CC1Oc2c(F)cc(C3CN(C(=O)OC(C)(C)C)C3)cc2N(C(=O)OC(C)(C)C)c2nccc(Cl)c21]

Intermediate 472 was synthesized in a similar manner as Intermediate 211 using Intermediate 471 instead of Intermediate 210 and [1-[(1,1-dimethylethoxy)carbonyl]-3-azetidinyl]iodozine [CAS: 206446-38-0] instead of [1-[(1,1-dimethylethoxy)carbonyl]-4-piperidinyl]iodozine.

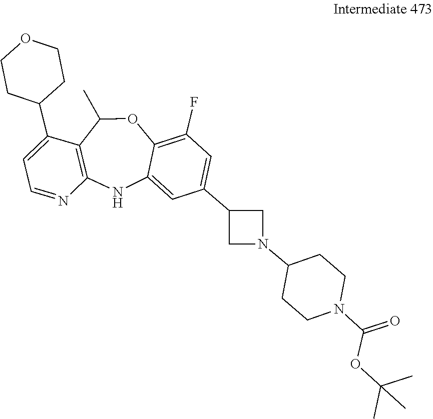 [SMILES: CC1Oc2c(F)cc(C3CN(C4CCN(C(=O)OC(C)(C)C)CC4)C3)cc2Nc2nccc(C3CCOCC3)c21]

Intermediate 473 was synthesized following the synthetic route from Intermediate 8 to Intermediate 9 starting with Intermediate 472 instead of Intermediate 7 and using tert-butyl-4-oxopiperidine-1-carboxylate [CAS: 79099-07-3] instead of 1-Boc-3-azetidinone.

### Intermediate 474A and Intermediate 474B

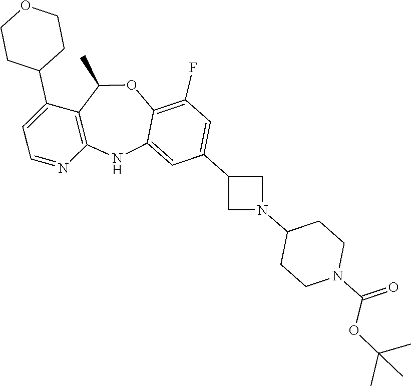 [SMILES: C[C@H]1Oc2c(F)cc(C3CN(C4CCN(C(=O)OC(C)(C)C)CC4)C3)cc2Nc2nccc(C3CCOCC3)c21]

Intermediate 474A: (*R, pure stereoisomer but absolute stereochemistry undetermined

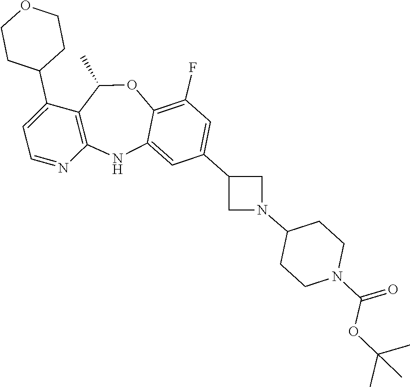 [SMILES: C[C@@H]1Oc2c(F)cc(C3CN(C4CCN(C(=O)OC(C)(C)C)CC4)C3)cc2Nc2nccc(C3CCOCC3)c21]

Intermediate 474B: (*S), pure stereoisomer but absolute stereochemistry undetermined Intermediate 473 (1.27 g, 2.3 mmol) was separated into its enantiomers by normal phase chiral chromatography (Phenomenex Lux Cellulose-1 250×30 mm 5 μm; 72% [heptane+0.1% DEA]−28% [iPrOH+0.1% DEA] to 36% [heptane+0.1% DEA]−64% [iPrOH+0.1% DEA]) to yield Intermediate 474A (541 mg, yield: 43%) and Intermediate 474B (491 mg, yield: 39%).

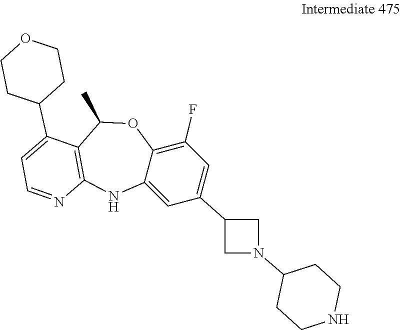 [SMILES: C[C@H]1Oc2c(F)cc(C3CN(C4CCNCC4)C3)cc2Nc2nccc(C3CCOCC3)c21]

(*R), pure stereoisomer but absolute stereochemistry undetermined Intermediate 475 was synthesized in a similar manner as Intermediate 53 using Intermediate 474A instead of Intermediate 52.

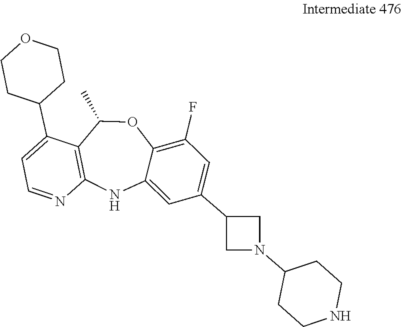 [SMILES: C[C@@H]1Oc2c(F)cc(C3CN(C4CCNCC4)C3)cc2Nc2nccc(C3CCOCC3)c21]

(*S), pure stereoisomer but absolute stereochemistry undetermined Intermediate 476 was synthesized in a similar manner as Intermediate 53 using Intermediate 474B instead of Intermediate 52.

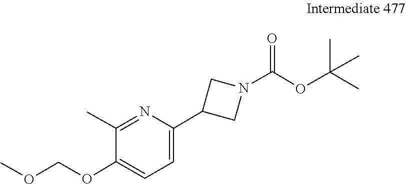 [SMILES: COCOc1ccc(C2CN(C(=O)OC(C)(C)C)C2)nc1C]

Intermediate 477 was synthesized in a similar manner as Intermediate 78 using 6-bromo-3-(methoxymethoxy)-2-methylpyridine [CAS: 1783265-24-6] instead of 2-chloro-5-(methoxymethoxy)pyridine and [1-[(1,1-dimethylethoxy)carbonyl]-3-azetidinyl]iodozine [CAS: 206446-38-0] instead of [1-(tert-butoxycarbonyl)piperidin-4-yl]zinc iodide.

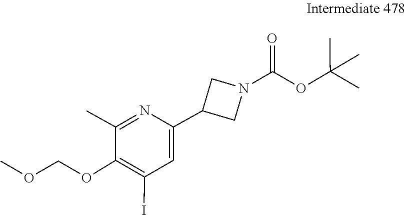 [SMILES: COCOc1c(I)cc(C2CN(C(=O)OC(C)(C)C)C2)nc1C]

Intermediate 477 (7 g, 22.7 mmol, 1.0 eq.) in THF (100 mL) was cooled at −78° C. under nitrogen atmosphere. nBuLi (2.5 M in hexanes, 10.9 mL, 27.2 mmol, 1.2 eq.) was added dropwise over 15 min and stirring was continued for 15 min. A solution of iodine (6.9 g, 27.2 mmol, 1.2 eq.) in THF (50 mL) was added dropwise over 15 min. The reaction mixture was stirred further for 1 h. The reaction mixture was diluted with water (100 mL), EtOAc (500 mL), and saturated aqueous Na₂S₂O₃ (10 mL). The organic layer was separated, washed with brine (20 mL), dried over MgSO₄, filtered, and concentrated. The residue was purified with column chromatography over silica gel (gradient of EtOAc in heptane from 0 to 50%) to afford Intermediate 478 (8.5 g, yield: 81%) as an oil.

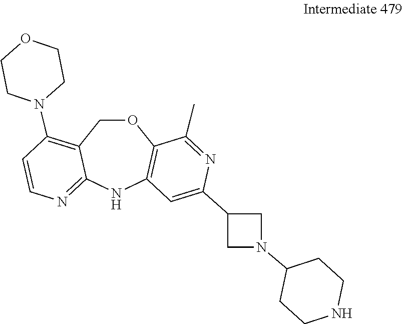 [SMILES: Cc1nc(C2CN(C3CCNCC3)C2)cc2c1OCc1c(N3CCOCC3)ccnc1N2]

Intermediate 479 was synthesized following the synthetic route from Intermediate 83 to Intermediate 89 starting with Intermediate 478 instead of Intermediate 82 and using tert-butyl 4-oxopiperidine-1-carboxylate [CAS: 79099-07-3] instead of 1-Boc-3-azetidinone.

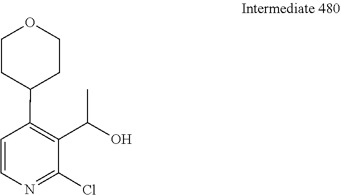 [SMILES: CC(O)c1c(C2CCOCC2)ccnc1Cl]

Intermediate 480 was synthesized in a similar manner as Intermediate 26 using Intermediate 359 instead of 2-chloro-4-(4-morpholinyl)-3-pyridinecarboxaldehyde.

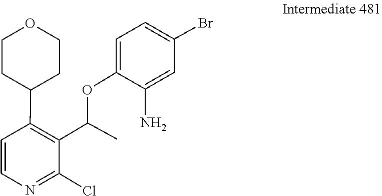 [SMILES: CC(Oc1ccc(Br)cc1N)c1c(C2CCOCC2)ccnc1Cl]

Intermediate 481 was synthesized following the synthetic route from Intermediate 141 to Intermediate 142 using Intermediate 480 instead of 2,4-dichloro-3-pyridinemethanol and 4-bromo-2-nitrophenol [CAS: 7693-52-9] instead of tert-butyl (4-hydroxy-3-nitrophenyl)carbamate.

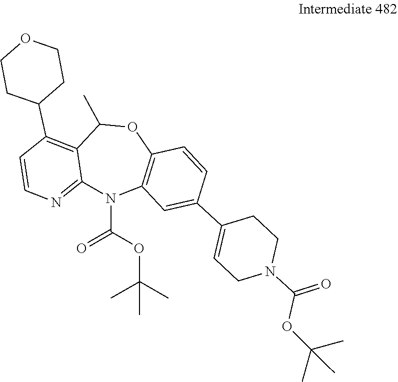 [SMILES: CC1Oc2ccc(C3=CCN(C(=O)OC(C)(C)C)CC3)cc2N(C(=O)OC(C)(C)C)c2nccc(C3CCOCC3)c21]

Intermediate 482 was synthesized following the synthetic route from Intermediate 18 to Intermediate 20 using Intermediate 481 instead of Intermediate 17.

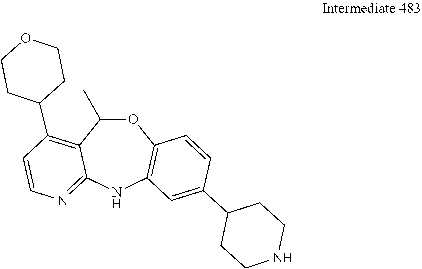 [SMILES: CC1Oc2ccc(C3CCNCC3)cc2Nc2nccc(C3CCOCC3)c21]

Intermediate 483 was synthesized following the synthetic route from Intermediate 7 to Intermediate 8 using Intermediate 482 instead of Intermediate 6.

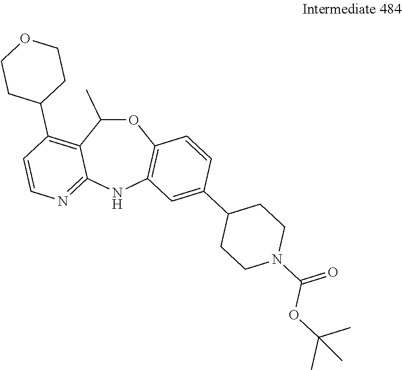 [SMILES: CC1Oc2ccc(C3CCN(C(=O)OC(C)(C)C)CC3)cc2Nc2nccc(C3CCOCC3)c21]

Boc-anhydride (565 mg, 2.6 mmol, 1.05 eq.) was added to a stirred solution of Intermediate 483 (935 mg, 2.5 mmol 1.0 eq.) and DMAP (30 mg, 0.25 mmol, 0.1 eq.) in anhydrous DCM (14 mL) at room temperature and the reaction mixture was stirred at room temperature for 2 h. The reaction mixture was washed with water and brine. The organic layer was dried (Na₂SO₄), filtered, and concentrated in vacuo to give Intermediate 484 (1.1 g, yield: 91%) as a pale yellow solid, used without further purification.

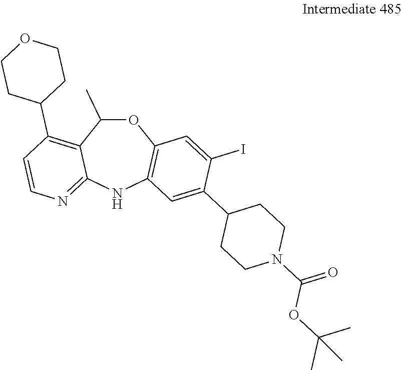 [SMILES: CC1Oc2cc(I)c(C3CCN(C(=O)OC(C)(C)C)CC3)cc2Nc2nccc(C3CCOCC3)c21]

NIS ([CAS: 516-12-1], 450 mg, 2.0 mmol, 1.2 eq.) was added portionwise to a stirred suspension of Intermediate 484 (800 mg, 1.7 mmol, 1.0 eq.) in HOAc (8.4 mL) at room temperature and the reaction mixture was stirred at room temperature for 40 min. The reaction mixture was quenched with saturated aqueous Na₂S₂O₃ and extracted with EtOAc. The organic layer was separated, washed with water, brine, dried (Na₂SO₄), filtered, and concentrated in vacuo. The residue was purified by flash column chromatography (silica; EtOAc in heptane, 0/100 to 50/50). The desired fractions were collected and concentrated in vacuo yielding Intermediate 485 (925 mg, yield: 92%) as a white solid.

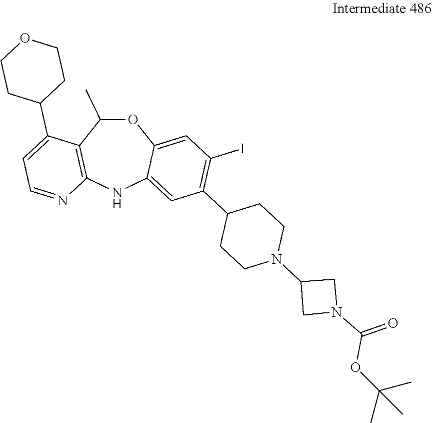 [SMILES: CC1Oc2cc(I)c(C3CCN(C4CN(C(=O)OC(C)(C)C)C4)CC3)cc2Nc2nccc(C3CCOCC3)c21]

Intermediate 486 was synthesized following the synthetic route from Intermediate 8 to Intermediate 9 using Intermediate 485 instead of Intermediate 7.

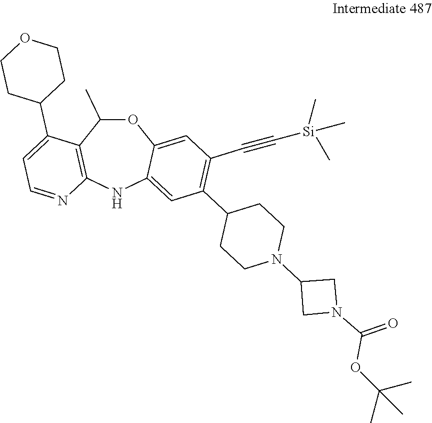 [SMILES: CC1Oc2cc(C#C[Si](C)(C)C)c(C3CCN(C4CN(C(=O)OC(C)(C)C)C4)CC3)cc2Nc2nccc(C3CCOCC3)c21]

Et₃N (1.16 mL, 8.3 mmol, 6.0 eq.) was added to a stirred suspension of Intermediate 485 (917 mg, 1.4 mmol, 1.0 eq.), trimethylsilylacetylene ([CAS: 1066-54-2], 0.6 mL, 4.17 mmol, 3.0 eq.), PdCl₂(PPh₃)₂ (99 mg, 0.14 mmol, 0.1 eq.), and CuI (14 mg, 0.075 mmol, 0.05 eq.) in anhydrous DMF (18 mL) under nitrogen and the reaction mixture was stirred at room temperature for 4 h. The reaction mixture was diluted with saturated aqueous NH₄Cl and extracted with EtOAc. The organic layer was washed with water, then brine, dried (Na₂SO₄), filtered, and concentrated in vacuo. The residue was purified by flash column chromatography (silica; EtOAc in heptane 0/100 to 100/00) to give Intermediate 487 (808 mg, yield: 92%) as a brown solid.

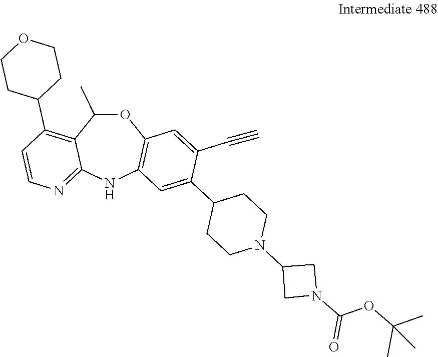 [SMILES: C#Cc1cc2c(cc1C1CCN(C3CN(C(=O)OC(C)(C)C)C3)CC1)Nc1nccc(C3CCOCC3)c1C(C)O2]

TBAF (1 M in THF, [CAS: 429-41-4], 1.1 mL, 1.1 mmol, 1.0 eq.) was added to a stirred solution of Intermediate 487 (703 mg, 1.1 mmol, 1.0 eq.) in anhydrous THF (11 mL) at room temperature and the reaction mixture was stirred at room temperature for 1 h. The reaction mixture was diluted with EtOAc and washed with water (×4). The organic layer was separated, dried (Na₂SO₄), filtered, and concentrated in vacuo. The residue was purified by flash column chromatography (silica, EtOAc in heptane 60/40 to 100/0) to yield Intermediate 488 (744 mg, quantitative yield) as a brown solid.

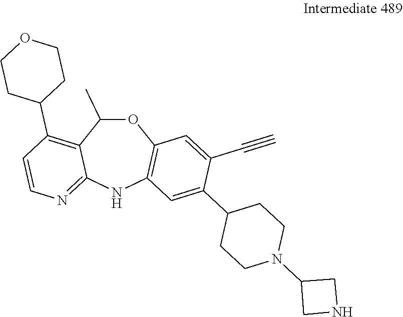 [SMILES: C#Cc1cc2c(cc1C1CCN(C3CNC3)CC1)Nc1nccc(C3CCOCC3)c1C(C)O2]

Trimethylsilyltrifluoromethanesulfonate ([CAS: 27607-77-8], 0.13 mL, 0.69 mmol, 2.0 eq.) was added to a stirred solution of Intermediate 488 (202 mg, 0.34 mmol, 1.0 eq.) in anhydrous DCM (3 mL) at 0° C. under nitrogen atmosphere. The reaction mixture was stirred at 0° C. for 1 h. The reaction mixture was concentrated in vacuo. The residue was purified by reverse phase HPLC (Stationary phase: C18 XBridge 30×100 mm 5 μm, Mobile phase: Gradient from 75% NH₄HCO₃ 0.25% solution in Water, 25% ACN to 57% NH₄HCO₃ 0.25% solution in water, 43% ACN), yielding Intermediate 489 (134 mg, yield: 86%) as a white solid.

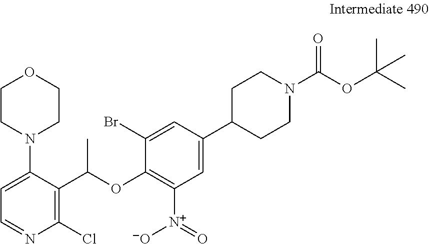 [SMILES: CC(Oc1c(Br)cc(C2CCN(C(=O)OC(C)(C)C)CC2)cc1[N+](=O)[O-])c1c(N2CCOCC2)ccnc1Cl]

DIAD (6.1 mL, 30.9 mmol, 1.5 eq.) was added dropwise to a stirred solution of Intermediate 26 (5 g, 20.6 mmol, 1.0 eq.), Intermediate 362 (9.7 g, 20.6 mmol, 1.0 eq.) and triphenylphosphine (8.1 g, 30.9 mmol, 1.5 eq.) in THF (100 mL) at room temperature under nitrogen atmosphere. The reaction mixture was stirred at room temperature for 16 h. The reaction mixture was diluted with EtOAc and washed with saturated aqueous NaHCO₃. The organic layer was separated, dried (MgSO₄), filtered, and concentrated in vacuo. The residue was suspended in DIPE, sonicated for 30 min, and filtered. The yellow solid obtained was purified by flash column chromatography (silica; EtOAc in heptane from 00/100 to 55/45) to yield Intermediate 490 (6.1 g, yield: 41%) as a yellow solid.

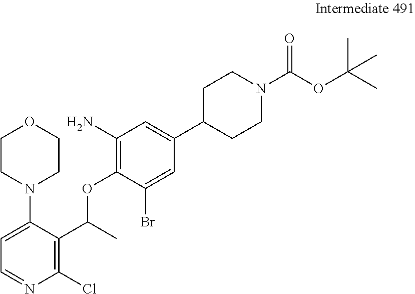 [SMILES: CC(Oc1c(N)cc(C2CCN(C(=O)OC(C)(C)C)CC2)cc1Br)c1c(N2CCOCC2)ccnc1Cl]

A solution of Intermediate 490 (2.37 g, 3.5 mmol, 1.0 eq.) in absolute EtOH (100 mL) and anhydrous THF (124 mL) was hydrogenated in a H-CUBE using Pt/C 10% (676 mg, 3.5 mmol, 1.0 eq.) as catalyst (1 mL/mm, 70 mm Pt/C 10% cartridge, Full-H₂ mode, 40° C., 7 cycles). The solvent was evaporated in vacuo. The residue was purified by flash column chromatography (silica, EtOAc in heptane 00/100 to 70/30) to yield Intermediate 491 (1.0 g, yield: 45%) as a white solid.

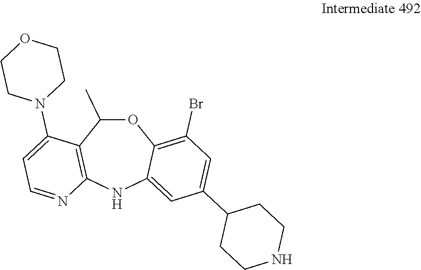 [SMILES: CC1Oc2c(Br)cc(C3CCNCC3)cc2Nc2nccc(N3CCOCC3)c21]

TFA (234 μL, 3.1 mmol, 3.0 eq.) was added to a solution of Intermediate 491 (650 mg, 1.0 mmol, 1.0 eq.) in anhydrous 1,4-dioxane (8 mL) in a sealed tube and the reaction mixture was stirred at 90° C. for 7 days. The reaction mixture was filtered and water was added to the filtrate. The mixture was extracted with 10% NH₃ (7 N in MeOH) in DCM. The organic layer was separated, dried (Na₂SO₄), filtered, and concentrated in vacuo to yield Intermediate 492 (476 mg, quantitative yield) as a grey solid, used without further purification.

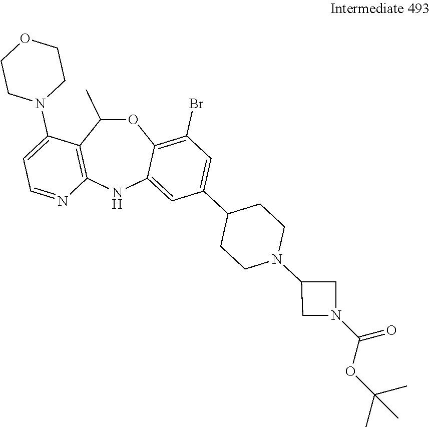 [SMILES: CC1Oc2c(Br)cc(C3CCN(C4CN(C(=O)OC(C)(C)C)C4)CC3)cc2Nc2nccc(N3CCOCC3)c21]

Intermediate 493 was synthesized in a similar manner as Intermediate 14 using Intermediate 492 instead of Intermediate 13.

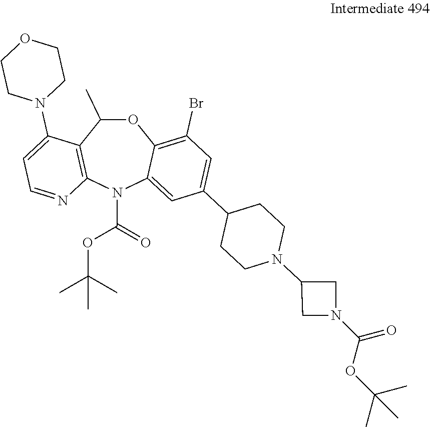 [SMILES: CC1Oc2c(Br)cc(C3CCN(C4CN(C(=O)OC(C)(C)C)C4)CC3)cc2N(C(=O)OC(C)(C)C)c2nccc(N3CCOCC3)c21]

Intermediate 494 was synthesized in a similar manner as Intermediate 19 using Intermediate 493 instead of Intermediate 18.

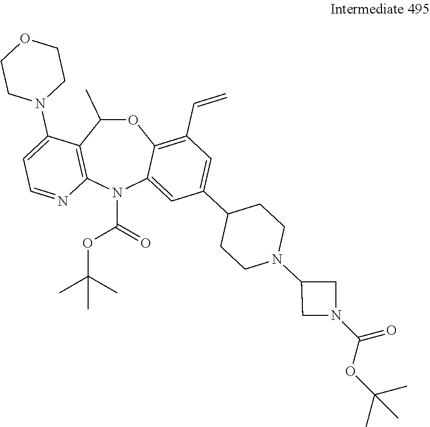 [SMILES: C=Cc1cc(C2CCN(C3CN(C(=O)OC(C)(C)C)C3)CC2)cc2c1OC(C)c1c(N3CCOCC3)ccnc1N2C(=O)OC(C)(C)C]

Pd(PPh₃)₄ (42 mg, 0.04 mmol, 0.1 eq.) was added to a stirred suspension of Intermediate 494 (265 mg, 0.37 mmol, 1.0 eq.) and potassium vinyltrifluoroborate (100 mg, 0.74 mmol, 2.0 eq.) in 1,4-dioxane (2 mL) and saturated Na₂CO₃ (530 μL, 1.1 mmol, 2.8 eq.) in a sealed tube and under nitrogen. The mixture was stirred at 120° C. for 20 min under microwave irradiation. The mixture was diluted with water and extracted with EtOAc. The organic layer was separated, washed with saturated NaCl, dried (Na₂SO₄), filtered, and concentrated in vacuo. The residue was purified by flash column chromatography (silica; MeOH in DCM 0/100 to 4/96) to yield Intermediate 495 (232 mg, yield: 80%) as a pale yellow solid.

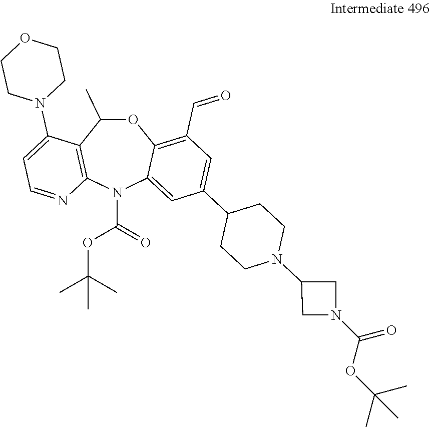 [SMILES: CC1Oc2c(C=O)cc(C3CCN(C4CN(C(=O)OC(C)(C)C)C4)CC3)cc2N(C(=O)OC(C)(C)C)c2nccc(N3CCOCC3)c21]

Osmium tetroxide (2.5 wt % solution in tert-butanol, 6 μL, 0.015 mmol, 0.065 eq.) was added to a stirred mixture of Intermediate 495 (183 mg, 0.24 mmol, 1.0 eq.), 2,6-dimethylpyridine (55 μL, 0.47 mmol, 2.0 eq.), and sodium periodate (201 mg, 0.94 mmol, 4.0 eq.) in 1,4-dioxane (2.5 mL) and distilled water (0.6 mL) at 0° C. The reaction mixture was stirred at room temperature for 1 h. The reaction mixture was basified with saturated aqueous NaHCO₃ and extracted with EtOAc. The organic layer was separated, dried (Na₂SO₄), filtered, and concentrated in vacuo to yield Intermediate 496 (203 mg, quantitative yield) as a colourless oil, used without further purification.

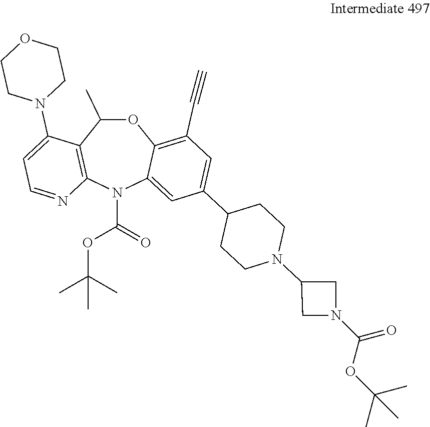 [SMILES: C#Cc1cc(C2CCN(C3CN(C(=O)OC(C)(C)C)C3)CC2)cc2c1OC(C)c1c(N3CCOCC3)ccnc1N2C(=O)OC(C)(C)C]

Dimethyl P-(1-diazo-2-oxopropyl)phosphonate ([CAS: 90965-06-3], 10% in ACN, 336 μL, 0.47 mmol, 2.0 eq.) was added dropwise to a mixture of Intermediate 496 (203 mg, 0.24 mmol, 1.0 eq.) and K₂CO₃ (98 mg, 0.71 mmol, 3.0 eq.) in THF (0.2 mL) and MeOH (0.2 mL) at room temperature. The reaction mixture was stirred at room temperature for 18 h. The reaction mixture was diluted with EtOAc and washed with saturated aqueous NaHCO₃. The organic layer was separated, dried (Na₂SO₄), filtered, and concentrated in vacuo. The residue was purified by flash column chromatography (silica; EtOAc in DCM 0/100 to 100/0) to afford Intermediate 497 (133 mg, yield: 74%) as a white solid.

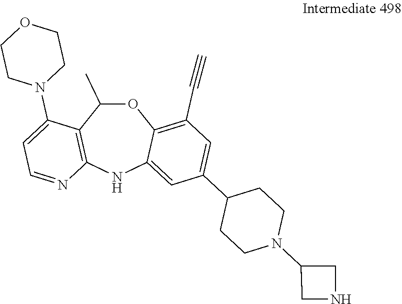 [SMILES: C#Cc1cc(C2CCN(C3CNC3)CC2)cc2c1OC(C)c1c(N3CCOCC3)ccnc1N2]

HCl (4 M in dioxane, 9 μL, 0.36 mmol, 3.0 eq.) was added to Intermediate 497 (93 mg, 0.12 mmol, 1.0 eq.) in 1,4-dioxane (1 mL) and the reaction mixture was stirred at room temperature for 27 h. The reaction mixture was treated with NH₃ (7 M in MeOH) and concentrated in vacuo. The residue was purified by reverse phase HPLC (Stationary phase: XBridge C18 50×100 mm, 5 μm, Mobile phase: Gradient from 55% NH₄HCO₃ 0.25% solution in Water, 45% MeOH to 35% NH₄HCO₃ 0.25% solution in Water, 65% MeOH) yielding Intermediate 498 (35 mg, yield: 63%) as a yellow solid.

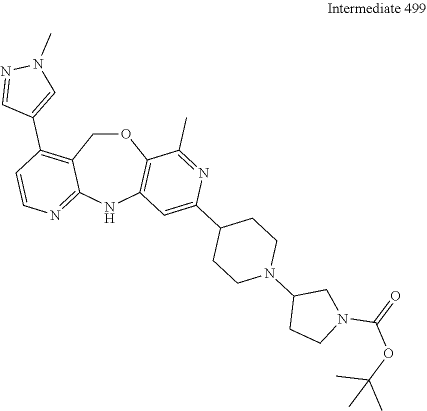 [SMILES: Cc1nc(C2CCN(C3CCN(C(=O)OC(C)(C)C)C3)CC2)cc2c1OCc1c(-c3cnn(C)c3)ccnc1N2]

Intermediate 499 was synthesized in a similar manner as Intermediate 49 using 3-oxopyrrolidine-1-carboxylic acid tert-butyl ester [CAS: 101385-93-7] instead of tert-butyl 3-oxoazetidine-1-carboxylate.

### Intermediate 500A and Intermediate 500B

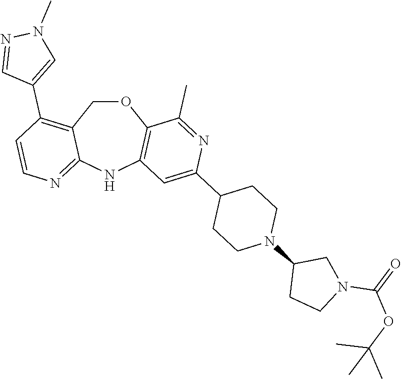 [SMILES: Cc1nc(C2CCN([C@@H]3CCN(C(=O)OC(C)(C)C)C3)CC2)cc2c1OCc1c(-c3cnn(C)c3)ccnc1N2] [0824] (*R), pure stereoisomer but absolute stereochemistry undetermined

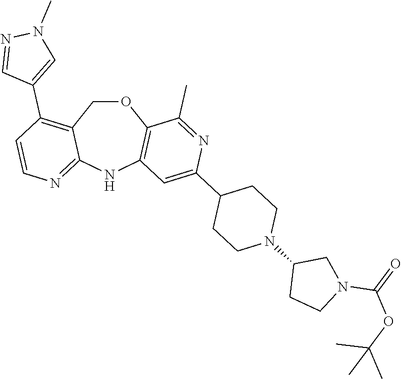 [SMILES: Cc1nc(C2CCN([C@H]3CCN(C(=O)OC(C)(C)C)C3)CC2)cc2c1OCc1c(-c3cnn(C)c3)ccnc1N2]

(*S), pure stereoisomer but absolute stereochemistry undetermined Intermediate 499 was separated into its stereoisomers by chiral column chromatography (Phenomenex Lux Amylose-1 150×21.2 mm 5 μm; gradient from 81% heptane with 0.1% DEA−19% iPrOH with 0.1% DEA to 45% heptane with 0.1% DEA−55% iPrOH with 0.1% DEA) to give Intermediate 500A (287 mg, yield: 22%) as a clear oil and Intermediate 500B (307 mg, yield: 23%) as a clear oil.

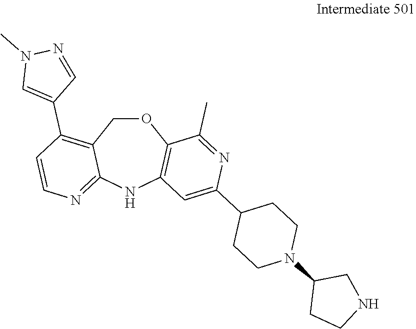 [SMILES: Cc1nc(C2CCN([C@@H]3CCNC3)CC2)cc2c1OCc1c(-c3cnn(C)c3)ccnc1N2]

(*R), pure stereoisomer but absolute stereochemistry undetermined Intermediate 501 was synthesized in a similar manner as Intermediate 50 using Intermediate 500A instead of Intermediate 49.

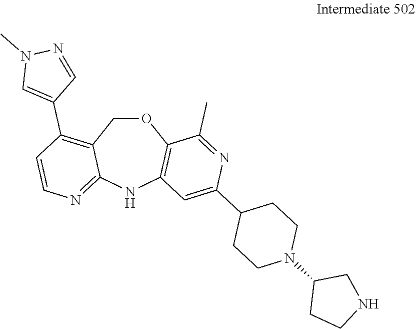 [SMILES: Cc1nc(C2CCN([C@H]3CCNC3)CC2)cc2c1OCc1c(-c3cnn(C)c3)ccnc1N2]

(*S), pure stereoisomer but absolute stereochemistry undetermined Intermediate 502 was synthesized in a similar manner as Intermediate 50 using Intermediate 500B instead of Intermediate 49.

### Intermediate 503A and Intermediate 503B

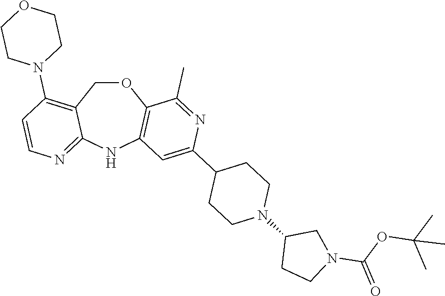 [SMILES: Cc1nc(C2CCN([C@H]3CCN(C(=O)OC(C)(C)C)C3)CC2)cc2c1OCc1c(N3CCOCC3)ccnc1N2]

Intermediate 503A: (*S), pure stereoisomer but absolute stereochemistry undetermined

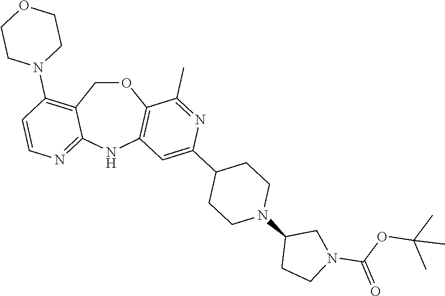 [SMILES: Cc1nc(C2CCN([C@@H]3CCN(C(=O)OC(C)(C)C)C3)CC2)cc2c1OCc1c(N3CCOCC3)ccnc1N2]

Intermediate 503B: (*R), pure stereoisomer but absolute stereochemistry undetermined Intermediate 503A and 503B were synthesized in a similar manner as Intermediate 64 using N-Boc pyrrolidin-3-one [CAS: 101385-93-7] instead of tert-butyl 3-oxoazetidine-1-carboxylate, followed by purification by chiral phase column chromatography (Phenomenex Lux Amylose-1 150×21.2 mm 5 μm; gradient from: 81% heptane with 0.1% DEA−19% iPrOH with 0.1% DEA to 45% heptane with 0.1% DEA−55% iPrOH with 0.1% DEA).

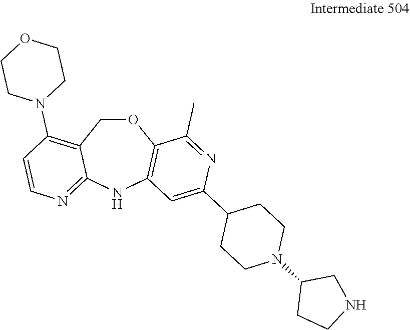 [SMILES: Cc1nc(C2CCN([C@H]3CCNC3)CC2)cc2c1OCc1c(N3CCOCC3)ccnc1N2]

(*S), pure stereoisomer but absolute stereochemistry undetermined Intermediate 504 was synthesized in a similar manner as Intermediate 50 using Intermediate 503A instead of Intermediate 49.

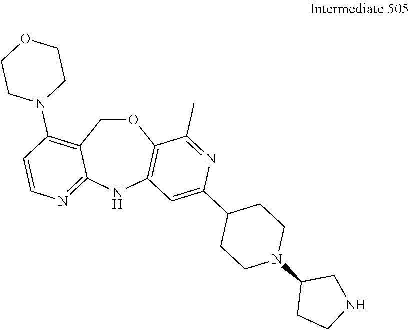 [SMILES: Cc1nc(C2CCN([C@@H]3CCNC3)CC2)cc2c1OCc1c(N3CCOCC3)ccnc1N2]

(*R), pure stereoisomer but absolute stereochemistry undetermined Intermediate 505 was synthesized in a similar manner as Intermediate 50 using Intermediate 503B instead of Intermediate 49.

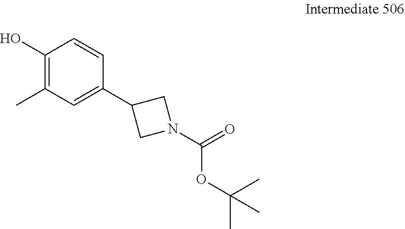 [SMILES: Cc1cc(C2CN(C(=O)OC(C)(C)C)C2)ccc1O]

Intermediate 506 was synthesized in a similar manner as Intermediate 327 using already activated [1-[(1,1-dimethylethoxy)carbonyl]-3-azetidinyl]iodozine [CAS: 206446-38-0] instead of Intermediate 326 and 4-bromo-2-methylphenol instead of Intermediate 231.

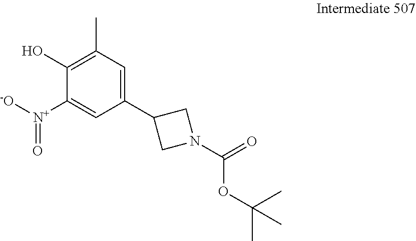

HNO₃ (65%, 9.7 mL, 144.5 mmol, 1.1 eq.) was added dropwise to a yellow suspension of Intermediate 506 (34.6 g, 131.4 mmol, 1.0 eq.) in AcOH (300 mL) at room temperature. The reaction mixture was stirred at room temperature for 10 min. The reaction mixture was diluted with EtOAc (700 mL), washed with saturated aqueous Na₂CO₃ (500 mL), H₂O (500 mL), and brine (1.5 mL). The combined organic layers were dried (MgSO₄), filtered, and concentrated. The residue was purified by flash column chromatography over 330 g silica gel (eluent: gradient EtOAc/petroleum ether from 0/100 to 26/74) to give Intermediate 507 (33.2 g, yield: 81%) as a yellow solid.

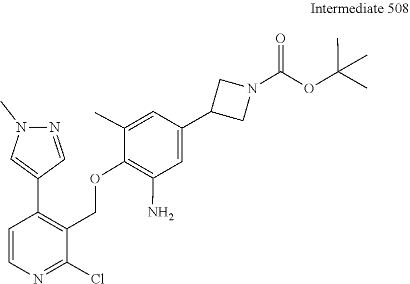 [SMILES: Cc1cc(C2CN(C(=O)OC(C)(C)C)C2)cc(N)c1OCc1c(-c2cnn(C)c2)ccnc1Cl]

Intermediate 508 was synthesized following the synthetic route from Intermediate 141 to Intermediate 142 using Intermediate 44 instead of 2,4-dichloro-3-pyridinemethanol and Intermediate 507 instead of tert-butyl (4-hydroxy-3-nitrophenyl)carbamate.

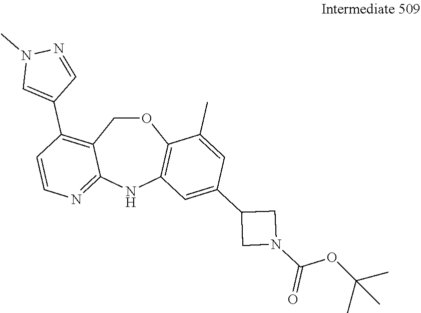 [SMILES: Cc1cc(C2CN(C(=O)OC(C)(C)C)C2)cc2c1OCc1c(-c3cnn(C)c3)ccnc1N2]

A mixture of Intermediate 508 (11 g, 21.1 mmol) in tert-amyl-alcohol (81 mL) was stirred at 140° C. for 1.5 h. The reaction mixture was cooled to room temperature and evaporated. The solid was triturated in ACN, filtered, and dried. The solid was taken up in DCM and the solution was washed with aqueous K₂CO₃ (10%). The organic layer was dried over MgSO₄, filtered, and dried to give Intermediate 509 (5 g, yield 53%).

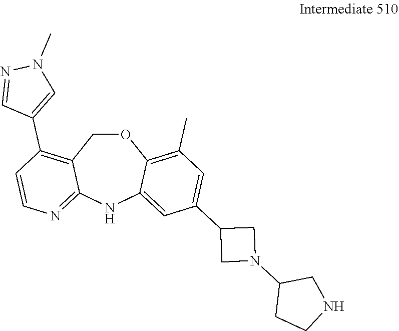 [SMILES: Cc1cc(C2CN(C3CCNC3)C2)cc2c1OCc1c(-c3cnn(C)c3)ccnc1N2]

Intermediate 510 was synthesized following the synthetic route of Intermediate 162 to Intermediate 164 starting with Intermediate 509 instead of Intermediate 161 and using N-Boc pyrrolidin-3-one [CAS: 101385-93-7] instead of 1-Boc-3-azetidinone.

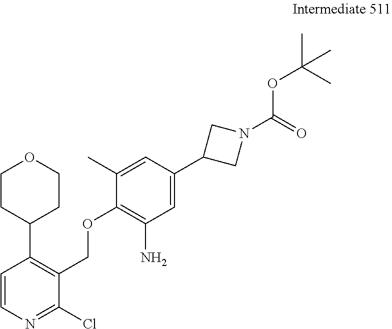 [SMILES: Cc1cc(C2CN(C(=O)OC(C)(C)C)C2)cc(N)c1OCc1c(C2CCOCC2)ccnc1Cl]

Intermediate 511 was synthesized following the synthetic route from Intermediate 141 to Intermediate 142 using Intermediate 408 instead of 2,4-dichloro-3-pyridinemethanol and Intermediate 507 instead of tert-butyl (4-hydroxy-3-nitrophenyl)carbamate.

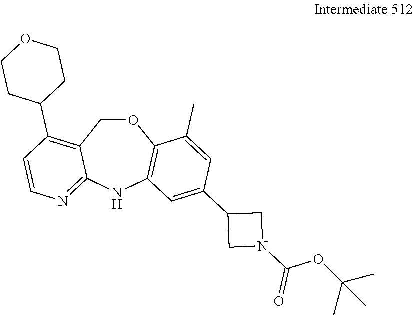 [SMILES: Cc1cc(C2CN(C(=O)OC(C)(C)C)C2)cc2c1OCc1c(C3CCOCC3)ccnc1N2]

Intermediate 512 was synthesized in a similar manner as Intermediate 509 using Intermediate 511 instead of Intermediate 508.

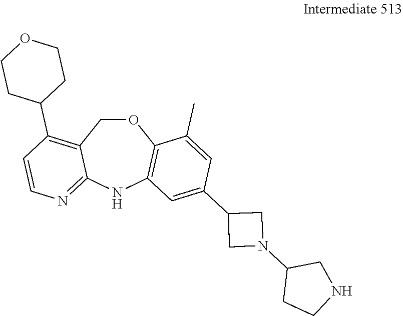 [SMILES: Cc1cc(C2CN(C3CCNC3)C2)cc2c1OCc1c(C3CCOCC3)ccnc1N2]

Intermediate 513 was synthesized following the synthetic route of Intermediate 162 to Intermediate 164 starting with Intermediate 510 instead of Intermediate 161 and using N-Boc pyrrolidin-3-one [CAS: 101385-93-7] instead of 1-Boc-3-azetidinone.

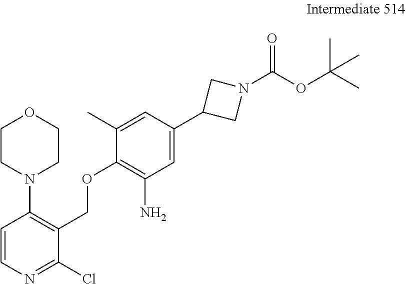 [SMILES: Cc1cc(C2CN(C(=O)OC(C)(C)C)C2)cc(N)c1OCc1c(N2CCOCC2)ccnc1Cl]

Intermediate 514 was synthesized following the synthetic route from Intermediate 141 to Intermediate 142 using Intermediate 59 instead of 2,4-dichloro-3-pyridinemethanol and Intermediate 507 instead of tert-butyl (4-hydroxy-3-nitrophenyl)carbamate.

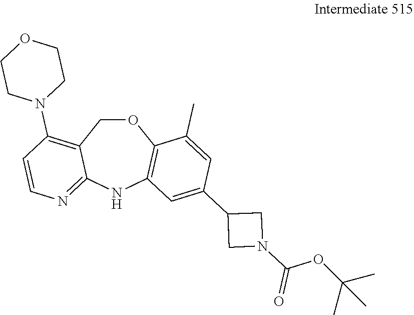 [SMILES: Cc1cc(C2CN(C(=O)OC(C)(C)C)C2)cc2c1OCc1c(N3CCOCC3)ccnc1N2]

Intermediate 515 was synthesized in a similar manner as Intermediate 509 using Intermediate 514 instead of Intermediate 508.

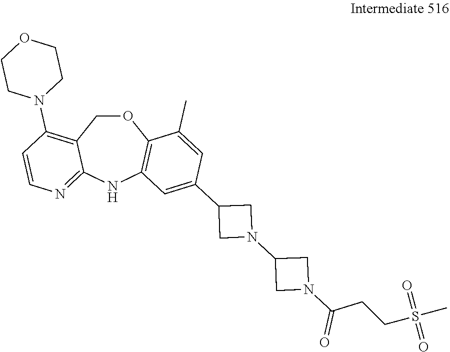 [SMILES: Cc1cc(C2CN(C3CN(C(=O)CCS(C)(=O)=O)C3)C2)cc2c1OCc1c(N3CCOCC3)ccnc1N2]

Intermediate 516 was synthesized following the synthetic route of Intermediate 8 to Intermediate 9 starting with Intermediate 515 instead of Intermediate 7 and using Intermediate 96 instead of 1-Boc-3-azetidinone.

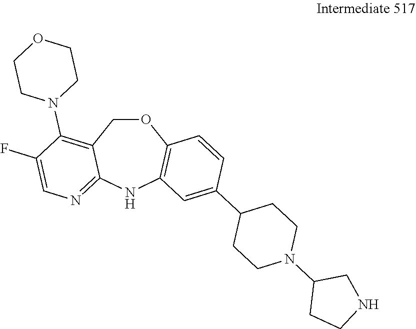 [SMILES: Fc1cnc2c(c1N1CCOCC1)COc1ccc(C3CCN(C4CCNC4)CC3)cc1N2]

Intermediate 517 was synthesized following the synthetic route from Intermediate 11 to Intermediate 15 starting with Intermediate 464 instead of Intermediate 4 and using N-Boc pyrrolidin-3-one [CAS: 101385-93-7] instead of 1-Boc-3-azetidinone.

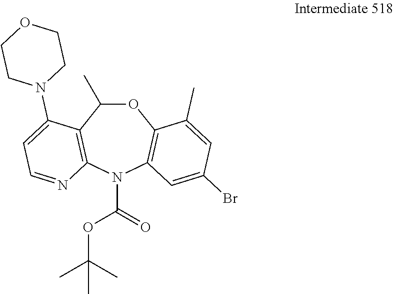 [SMILES: Cc1cc(Br)cc2c1OC(C)c1c(N3CCOCC3)ccnc1N2C(=O)OC(C)(C)C]

Intermediate 518 was synthesized following the synthetic route from Intermediate 16 to Intermediate 19 starting from Intermediate 26 instead of 2,4-dichloro-3-pyridinemethanol.

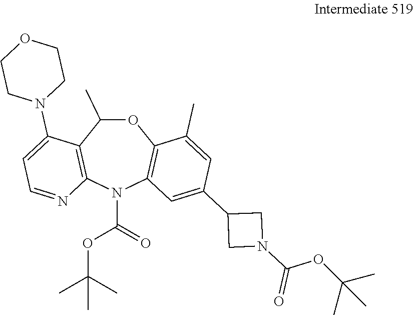 [SMILES: Cc1cc(C2CN(C(=O)OC(C)(C)C)C2)cc2c1OC(C)c1c(N3CCOCC3)ccnc1N2C(=O)OC(C)(C)C]

Intermediate 519 was synthesized in a similar manner as Intermediate 211 using Intermediate 518 instead of Intermediate 210 and [1-[(1,1-dimethylethoxy)carbonyl]-3-azetidinyl]iodozine [CAS: 206446-38-0] instead of [1-[(1,1-dimethylethoxy)carbonyl]-4-piperidinyl]iodozine.

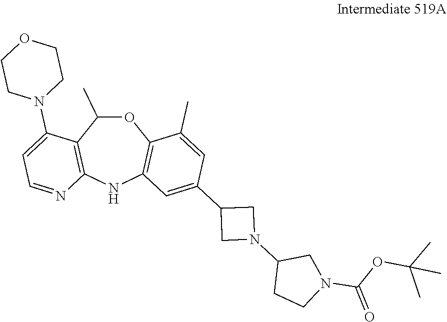 [SMILES: Cc1cc(C2CN(C3CCN(C(=O)OC(C)(C)C)C3)C2)cc2c1OC(C)c1c(N3CCOCC3)ccnc1N2]

Intermediate 519A was synthesized following the synthetic route from Intermediate 8 to Intermediate 9 using Intermediate 519 instead of Intermediate 7 and N-Boc pyrrolidin-3-one [CAS: 101385-93-7] instead of 1-Boc-3-azetidinone.

### Intermediate 520A, Intermediate 520B, Intermediate 520C and Intermediate 520D

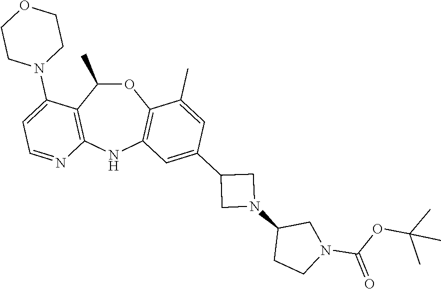 [SMILES: Cc1cc(C2CN([C@@H]3CCN(C(=O)OC(C)(C)C)C3)C2)cc2c1O[C@H](C)c1c(N3CCOCC3)ccnc1N2]

Intermediate 520A (7*R, 20*R), pure isomer but absolute stereochemistry undetermined

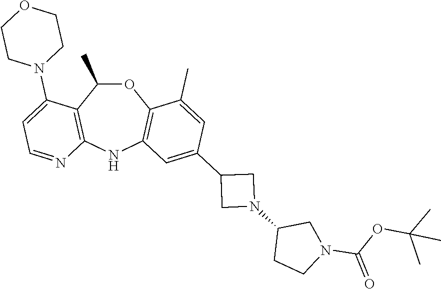 [SMILES: Cc1cc(C2CN([C@H]3CCN(C(=O)OC(C)(C)C)C3)C2)cc2c1O[C@H](C)c1c(N3CCOCC3)ccnc1N2]

Intermediate 520B (7*R, 20*S), pure isomer but absolute stereochemistry undetermined

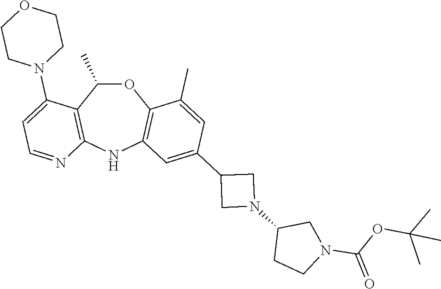 [SMILES: Cc1cc(C2CN([C@H]3CCN(C(=O)OC(C)(C)C)C3)C2)cc2c1O[C@@H](C)c1c(N3CCOCC3)ccnc1N2]

Intermediate 520C (7*S, 20*S), pure isomer but absolute stereochemistry undetermined

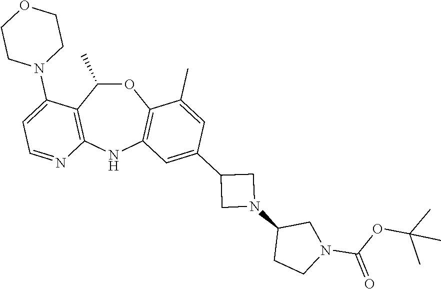 [SMILES: Cc1cc(C2CN([C@@H]3CCN(C(=O)OC(C)(C)C)C3)C2)cc2c1O[C@@H](C)c1c(N3CCOCC3)ccnc1N2]

Intermediate 520D (7*S, 20*R), pure isomer but absolute stereochemistry undetermined

Intermediate 519A (690 mg, 1.3 mmol) was separated into its stereoisomers by 3 successive chiral chromatography methods (Phenomenex Lux Amylose-1 250×30 mm 5 um; gradient from 75% [heptane+0.1% DEA]−25% [iPrOH+0.1% DEA] to 38% [heptane+0.1% DEA]−62% [iPrOH+0.1% DEA], followed by Phenomenex Lux Amylose-1 250×30 mm 5 μm; gradient from 75% [heptane+0.1% DEA]−25% [iPrOH+0.1% DEA] to 100% [iPrOH+0.1% DEA], and finally Phenomenex Lux Cellulose-1 250×30 mm 5 μm; gradient from 95% [heptane+0.1% DEA]−5% [iPrOH-EtOH (9:1)+0.1% DEA] to 30% [heptane+0.1% DEA]−70% [iPrOH-EtOH (9:1)+0.1% DEA]) to yield Intermediate 520A (98 mg, yield: 14%), Intermediate 520B (94 mg, yield: 14%), Intermediate 520C (138 mg, yield: 20%) and Intermediate 520D (115 mg, yield: 17%).

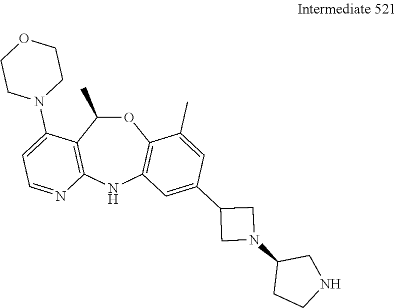 [SMILES: Cc1cc(C2CN([C@@H]3CCNC3)C2)cc2c1O[C@H](C)c1c(N3CCOCC3)ccnc1N2]

(7*R, 20*R), pure isomer but absolute stereochemistry undetermined Intermediate 521 was synthesized in a similar manner as Intermediate 38 using Intermediate 520A instead of Intermediate 37.

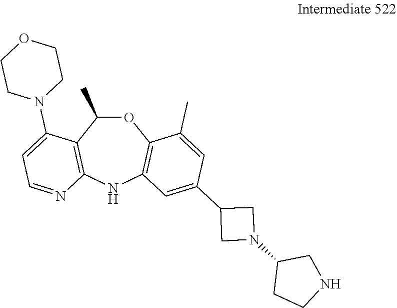 [SMILES: Cc1cc(C2CN([C@H]3CCNC3)C2)cc2c1O[C@H](C)c1c(N3CCOCC3)ccnc1N2]

(7*R, 20*S), pure isomer but absolute stereochemistry undetermined Intermediate 522 was synthesized in a similar manner as Intermediate 38 using Intermediate 520B instead of Intermediate 37.

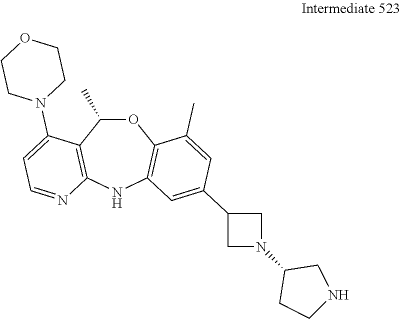 [SMILES: Cc1cc(C2CN([C@H]3CCNC3)C2)cc2c1O[C@@H](C)c1c(N3CCOCC3)ccnc1N2]

(7*S, 20*S), pure isomer but absolute stereochemistry undetermined Intermediate 523 was synthesized in a similar manner as Intermediate 38 using Intermediate 520C instead of Intermediate 37.

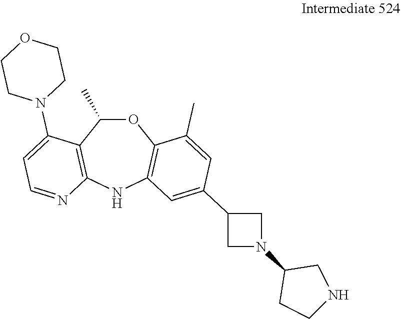 [SMILES: Cc1cc(C2CN([C@@H]3CCNC3)C2)cc2c1O[C@@H](C)c1c(N3CCOCC3)ccnc1N2]

(7*S, 20*R), pure isomer but absolute stereochemistry undetermined Intermediate 524 was synthesized in a similar manner as Intermediate 38 using Intermediate 520D instead of Intermediate 37.

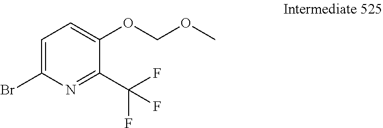

NaH (60% in mineral oil; 37 mg, 0.93 mmol, 1.2 eq.) was added to a solution of 6-bromo-2-(trifluoromethyl) pyridine-3-ol [CAS: 1227593-43-2] (190 mg, 0.78 mmol, 1 eq.) in DMF (2 mL) at 0° C. under nitrogen atmosphere. The mixture was stirred for 10 min, before the dropwise addition of chloromethyl methyl ether [CAS: 107-30-2] (71 μL, 0.93 mmol, 1.2 eq.). Stirring was continued for 1 h at room temperature. The reaction was quenched with water (50 mL) and the mixture was extracted with EtOAc. The combined organic layers were washed with brine, dried over MgSO₄, filtered, and concentrated. Column flash chromatography over silica gel (EtOAc in heptane from 0 to 20%) afforded Intermediate 525 as a clear oil (143 mg, yield: 64%).

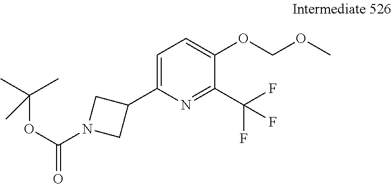 [SMILES: COCOc1ccc(C2CN(C(=O)OC(C)(C)C)C2)nc1C(F)(F)F]

Intermediate 525 (1.77 g, 6.19 mmol) was dissolved in DMA (20 mL) and bubbled with nitrogen for 5 min, then Pd(dppf)Cl₂. DCM [CAS: 95464-05-4] (0.25 g, 0.31 mmol, 0.05 eq.) and CuI [CAS: 7681-65-4] (0.12 g, 0.62 mmol, 0.1 eq.) were added to the mixture under nitrogen atmosphere. [1-[(1,1-Dimethylethoxy)carbonyl]-3-azetidinyl]iodozine [CAS: 206446-38-0] (0.44 M in DMA, 28 mL, 12.38 mmol, 2 eq.) was added and the resulting mixture was heated at 80° C. overnight under a nitrogen stream. The mixture was allowed to cool to room temperature and diluted with EtOAc (100 mL). Water (50 mL) and aqueous NH₃ (20 mL) were added. The organic layer was separated, washed with brine (50 mL), dried over MgSO₄, filtered, and concentrated to dryness. Column flash chromatography over silica gel (EtOAc in heptane from 0 to 100%) afforded Intermediate 526 as a solid (2.06 g, yield: 86%).

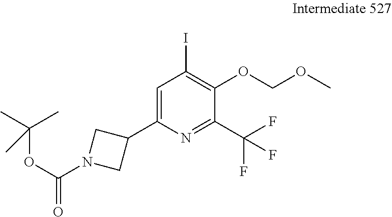 [SMILES: COCOc1c(I)cc(C2CN(C(=O)OC(C)(C)C)C2)nc1C(F)(F)F]

Intermediate 526 (2.06 g, 5.69 mmol) was dissolved in THF (48 mL) and cooled to −78° C. under nitrogen atmosphere. n-BuLi [CAS: 109-72-8] (2.5 M in hexanes, 2.73 mL, 6.83 mmol, 1.2 eq.) was added dropwise over 15 min and the stirring was continued for 15 min. A solution of iodine (1.73 g, 6.83 mmol, 1.2 eq.) in THF (12 mL) was then added dropwise over 15 min. The reaction mixture was stirred for 1 h. The reaction was quenched with water (25 mL). EtOAc (50 mL) and saturated aqueous Na₂S₂O₃ (15 mL) were added. The organic layer was separated, dried over MgSO₄, filtered, and concentrated in vacuo. Chromatography over silica gel (EtOAc in heptane from 0 to 50%) afforded Intermediate 527 as a brown oil (2.28 g, yield: 81%).

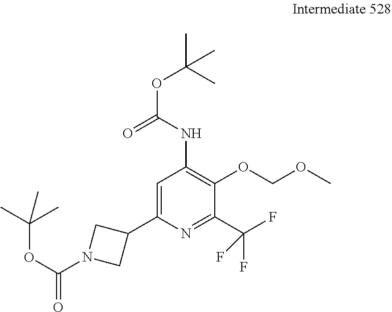 [SMILES: COCOc1c(NC(=O)OC(C)(C)C)cc(C2CN(C(=O)OC(C)(C)C)C2)nc1C(F)(F)F]

Intermediate 527 (2.28 g, 4.66 mmol, 1 eq.), tert-butyl carbamate (CAS: [4248-19-5]) (0.66 g, 5.6 mmol, 1.2 eq.), Pd₂(dba)₃ (0.13 g, 0.14 mmol, 0.03 eq.), Xantphos (CAS: [161265-03-8], 0.16 g, 0.28 mmol, 0.06 eq.), and Cs₂CO₃ (3.04 g, 9.33 mmol, 2 eq.) were suspended in toluene (25 mL) and the mixture was degassed by bubbling nitrogen for 15 min. The mixture was then heated at 100° C. for 3 h. The mixture was allowed to cool to room temperature and concentrated to half of its volume. Water was added and the mixture was extracted with EtOAc. The combined organic layers were washed with brine, dried over MgSO₄, filtered, and concentrated to dryness. Chromatography over silica gel (gradient of EtOAc in heptane from 0 to 33%) afforded Intermediate 528 as a solid (1.96 g, yield: 87%).

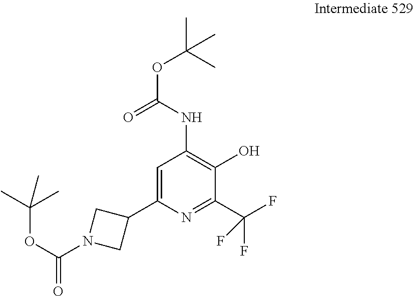 [SMILES: CC(C)(C)OC(=O)Nc1cc(C2CN(C(=O)OC(C)(C)C)C2)nc(C(F)(F)F)c1O]

HCl (37%, 390 μL, 4.66 mmol, 1.2 eq.) was added to a solution of Intermediate 528 (1.86 g, 3.89 mmol, 1 eq.) in iPrOH (40 mL). The reaction mixture was stirred overnight. Water and saturated aqueous NaHCO₃ were added until pH=7. The mixture was extracted with DCM. The organic layer was dried over MgSO₄, filtered, and concentrated to dryness to give Intermediate 529 (1.68 g, yield: 98%) as an oil, used without further purification.

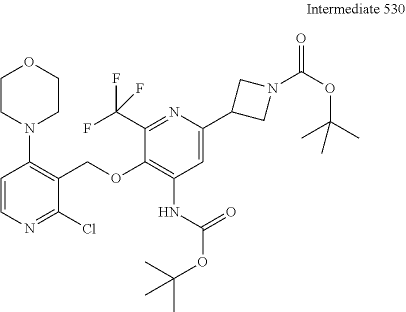 [SMILES: CC(C)(C)OC(=O)Nc1cc(C2CN(C(=O)OC(C)(C)C)C2)nc(C(F)(F)F)c1OCc1c(N2CCOCC2)ccnc1Cl]

Intermediate 60 (1.44 g, 5.82 mmol, 1.5 eq.) was added to a mixture of Intermediate 529 (1.68 g, 3.88 mmol, 1 eq.) and K₂CO₃ (0.7 g, 5.05 mmol, 1.3 eq.) in DMF (15 mL). The reaction was stirred at room temperature overnight, then H₂O and EtOAc were added. The organic layer was separated, dried over MgSO₄, filtered, and concentrated to dryness. Chromatography over silica gel (gradient of EtOAc in heptane from 0 to 100%) afforded Intermediate 530 as an oil (1.94 g, yield: 75%.

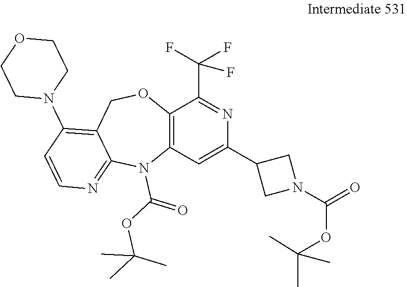 [SMILES: CC(C)(C)OC(=O)N1CC(c2cc3c(c(C(F)(F)F)n2)OCc2c(N4CCOCC4)ccnc2N3C(=O)OC(C)(C)C)C1]

Intermediate 530 (1.94 g, 2.86 mmol, 1 eq.) and Cs₂CO₃ (1.40 g, 4.29 mmol, 1.5 eq.) were suspended in 1,4-dioxane (50 mL) and bubbled with nitrogen for 15 min. Pd₂(dba)₃ [CAS: 51364-51-3;] (0.26 g, 0.29 mmol, 0.01 eq.) and Xantphos [CAS: 161265-03-8] (0.33 g, 0.57 mmol, 0.2 eq.) were then added and the resulting mixture was stirred at reflux overnight. The reaction mixture was then cooled to room temperature, diluted with H₂O, and extracted with EtOAc. The combined organic layers were washed with brine, dried over MgSO₄, filtered, concentrated to dryness and purified over silica gel (gradient of MeOH in DCM from 0 to 10%) to afford Intermediate 531 as a foam (1.44 g, yield: 81%).

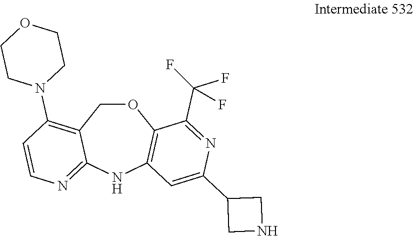 [SMILES: FC(F)(F)c1nc(C2CNC2)cc2c1OCc1c(N3CCOCC3)ccnc1N2]

TFA (1.81 mL, 23.68 mmol, 10 eq.) was added to a solution of Intermediate 531 (1.44 g, 2.37 mmol, 1 eq.) in DCM (30 mL) and the mixture was stirred at room temperature overnight. The reaction mixture was concentrated to dryness and the residue was washed with toluene twice and dried in vacuo to give Intermediate 532 (TFA salt, 2114 mg, quantitative) as an oil, used without further purification.

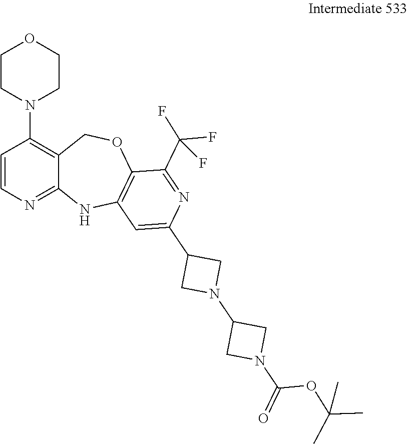 [SMILES: CC(C)(C)OC(=O)N1CC(N2CC(c3cc4c(c(C(F)(F)F)n3)OCc3c(N5CCOCC5)ccnc3N4)C2)C1]

Sodium triacetoxyborohydride [CAS: 56553-60-7] (0.39 g, 1.85 mmol, 2 eq.) was added to a solution of Intermediate 532 (0.8 g, 0.93 mmol, 1 eq.), Et₃N (0.39 mL, 2.78 mmol, 3 eq.), and 1-Boc-3-azetidinone [CAS: 398489-26-4] (0.24 g, 1.39 mmol, 1.5 eq.) in 1,2-DCE (20 mL). The mixture was stirred at room temperature overnight. Aqueous Na₂CO₃ (1 M) was added and the reaction mixture was extracted with DCM. The combined organic layer was dried over MgSO₄, filtered, and concentrated to dryness. Purification by silica gel chromatography (gradient of MeOH in DCM from 0% to 10%) gave Intermediate 533 as an oil (363 mg, yield: 69%).

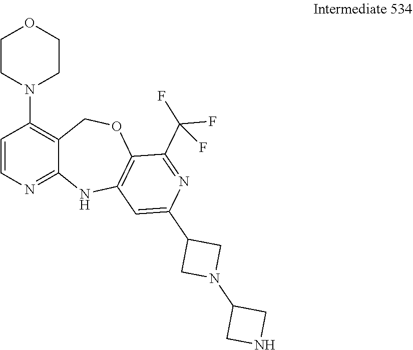 [SMILES: FC(F)(F)c1nc(C2CN(C3CNC3)C2)cc2c1OCc1c(N3CCOCC3)ccnc1N2]

TFA (0.99 mL, 12.9 mmol, 20 eq.) was added to a solution of Intermediate 533 (363 mg, 0.65 mmol, 1 eq.) in DCM (10 mL) and the mixture was stirred at room temperature overnight. The reaction mixture was concentrated to dryness and the residue was washed with toluene twice. Water was added to the residue and Na₂CO₃ was added until pH=7-8. The mixture was extracted with a mixture of DCM and MeOH (9:1). The organic layer was dried over MgSO₄, filtered, and concentrated to dryness to give Intermediate 534 (284 mg, yield: 93%) as an oil, used without further purification.

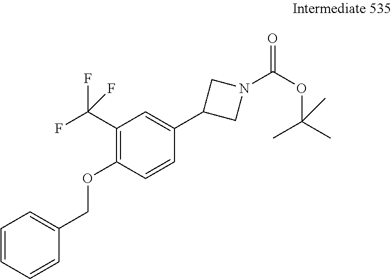 [SMILES: CC(C)(C)OC(=O)N1CC(c2ccc(OCc3ccccc3)c(C(F)(F)F)c2)C1]

1-(Benzyloxy)-4-bromo-2-(trifluoromethyl)benzene (CAS [169247-46-5], 4.67 g, 14.1 mmol, 1 eq.) was dissolved in DMA (45 mL) and the solution was bubbled with nitrogen for 5 min, then Pd(dppf)C12.DCM (0.58 g, 0.7 mmol, 0.05 eq.) and CuI (0.27 g, 1.41 mmol, 0.1 eq.) were added. [1-[(1,1-Dimethylethoxy)carbonyl]-3-azetidinyl]iodozine [CAS: 206446-38-0] (0.47 M solution in DMA, 60 mL, 28.19 mmol, 2 eq) was added and the resulting mixture was heated at 80° C. overnight. The mixture was allowed to cool to room temperature and was diluted with EtOAc (100 mL). Water (50 mL) and aqueous NH₃ (20 mL) were added. The organic layer was separated and washed with brine (50 mL), dried over MgSO₄, filtered, and concentrated to dryness. Column flash chromatography over silica gel (EtOAc in heptane from 0 to 100%) afforded Intermediate 535 as an oil (5.55 g, yield: 68%).

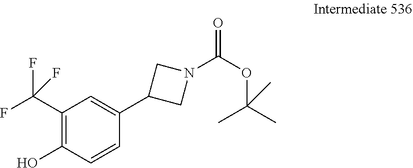 [SMILES: CC(C)(C)OC(=O)N1CC(c2ccc(O)c(C(F)(F)F)c2)C1]

Intermediate 535 (24.61 g, 42.28 mmol, 1 eq.) was dissolved in MeOH (200 mL) and cooled in an ice bath under a nitrogen stream. Pd/C (10%, 2.73 g, 25.67 mmol) was added and the reaction mixture was stirred at room temperature under H₂ atmosphere for 6 h. The mixture was filtered, over celite and evaporated until dryness to yield Intermediate 536 (1412 g, yield: 100%), used without further purification.

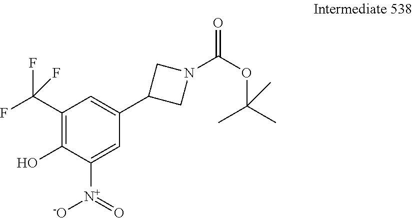

Intermediate 536 (4.32 g, 13.61 mmol, 1 eq.) was dissolved in AcOH (21 mL). Nitric acid (1 mL) was added dropwise and the resulting mixture was stirred at room temperature for a week. Additions of 1 mL of nitric acid to the reaction mixture were done every 24 h. The mixture was cooled with ice and neutralized with aqueous Na₂CO₃ (1 M) to pH=7. DCM was added and the layers were separated. Boc anhydride (3.27 g, 14.97 mmol) in DCM (150 mL) was added to the aqueous solution and the mixture was stirred at room temperature for 2 h. The layers were separated and the aqueous layer was extracted once more with DCM (50 mL). The combined organic layers were dried over MgSO₄, filtered, and evaporated in vacuo to afford Intermediate 538 (1.81 g, yield: 36%).

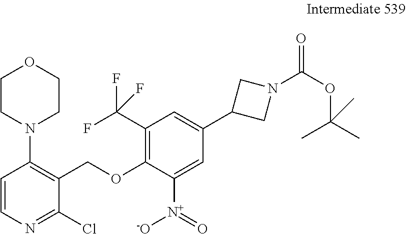

Intermediate 59 (1.14 g, 4.98 mmol, 1 eq.), Intermediate 538 (1.81 g, 4.98 mmol, 1 eq.) and triphenylphosphine [CAS: 603-35-0] (1.7 g, 6.48 mmol, 1.3 eq.) were suspended in THF (30 mL) under nitrogen atmosphere. A solution of DIAD (CAS: 2446-83-5) (1.28 mL, 6.48 mmol, 1.3 eq.) in THF (10 mL) was then added dropwise and the mixture was stirred for 3 h. The solvent was evaporated in vacuo and purification by flash column chromatography (silica; EtOAc in heptane 0/100 to 35/65) afforded Intermediate 539 (2.11 g, yield: 71%).

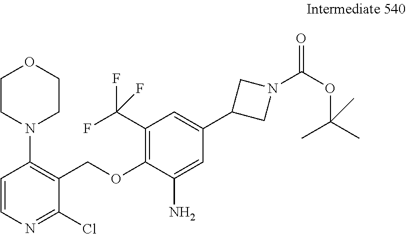 [SMILES: CC(C)(C)OC(=O)N1CC(c2cc(N)c(OCc3c(N4CCOCC4)ccnc3Cl)c(C(F)(F)F)c2)C1]

Iron (2.06 g, 36.88 mmol) was added to a mixture of Intermediate 539 (2.11 g, 3.69 mmol) and AcOH (4.22 mL, 73.76 mmol, 10 eq.) in MeOH (30 mL). The reaction mixture was stirred at room temperature overnight. DCM and saturated aqueous NaHCO₃ were added and the mixure was filtered through a short pad of celite. The layers were separated. The organic layer was dried over MgSO₄, filtered, and concentrated to dryness. Purification by column chromatography over silica gel (gradient of MeOH in DCM from 0% to 10%) afforded Intermediate 540 as an oil (1.91 g, yield: 91%).

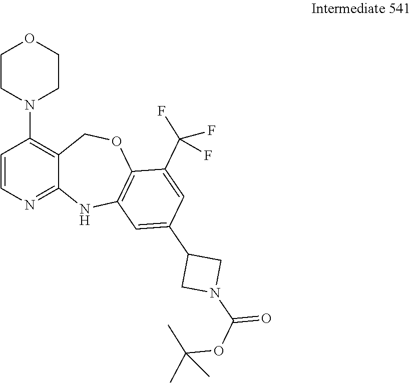 [SMILES: CC(C)(C)OC(=O)N1CC(c2cc3c(c(C(F)(F)F)c2)OCc2c(N4CCOCC4)ccnc2N3)C1]

Intermediate 540 (1.91 g, 3.34 mmol, 1 eq.) and Cs₂CO₃ (1.63 g, 5.02 mmol, 1.5 eq.) were suspended in 1,4-dioxane (60 mL) and bubbled with nitrogen for 15 min. Pd₂(dba)₃ [CAS: 51364-51-3] (306 mg, 0.33 mmol, 0.01 eq.) and Xantphos [CAS: 161265-03-8] (387 mg, 0.67 mmol, 0.2 eq.) were then added and the resulting mixture was stirred at reflux overnight. The reaction mixture was then cooled to room temperature, diluted with H₂O and extracted with EtOAc. The combined organic layers were washed with brine, dried over MgSO₄, filtered, concentrated to dryness and purified over silica gel (gradient of MeOH in DCM from 0 to 10%) to afford Intermediate 541 as a solid (1.07 g, yield: 60%).

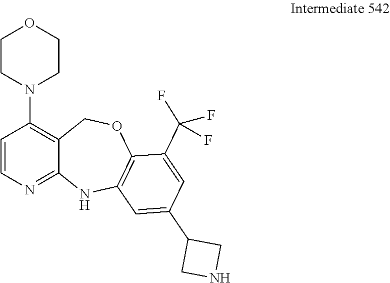 [SMILES: FC(F)(F)c1cc(C2CNC2)cc2c1OCc1c(N3CCOCC3)ccnc1N2]

TFA (1.55 mL, 20.28 mmol, 10 eq.) was added to a solution of Intermediate 541 (1.03 g, 2.03 mmol, 1 eq.) in DCM (15 mL) and the mixture was stirred at room temperature overnight. The reaction mixture was concentrated to dryness and the residue was washed with toluene twice and dried in vacuo to give Intermediate 542 (1.65 g, yield: 91%) as an oil, used without further purification.

### Intermediate 543A and Intermediate 543B

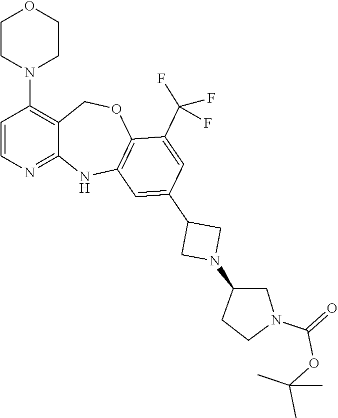 [SMILES: CC(C)(C)OC(=O)N1CC[C@@H](N2CC(c3cc4c(c(C(F)(F)F)c3)OCc3c(N5CCOCC5)ccnc3N4)C2)C1]

Intermediate 543A: (*R), pure enantiomer but absolute stereochemistry undetermined

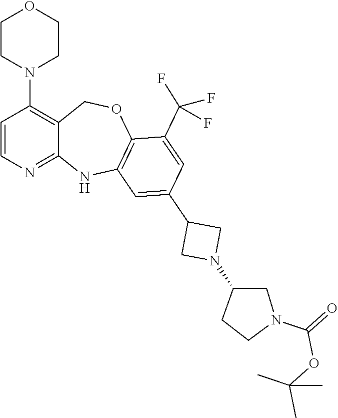 [SMILES: CC(C)(C)OC(=O)N1CC[C@H](N2CC(c3cc4c(c(C(F)(F)F)c3)OCc3c(N5CCOCC5)ccnc3N4)C2)C1]

Intermediate 543B: (*S), pure enantiomer but absolute stereochemistry undetermined Sodium triacetoxyborohydride [CAS: 56553-60-7] (0.86 g, 4.06 mmol, 2 eq.) was added to a solution of Intermediate 542 (1.75 g, 2.03 mmol, 1 eq.), Et₃N (0.85 mL, 6.08 mmol, 3 eq.), and N-Boc-3-pyrrolidinone [CAS: 101385-93-7] (0.56 g, 3.04 mmol, 1.5 eq.) in 1,2-DCE (40 mL). The mixture was stirred at room temperature overnight. Aqueous Na₂CO₃ (1 M) was added and the reaction mixture was extracted with DCM. The combined organic layer was dried over MgSO₄, filtered, and concentrated to dryness. Purification by silica gel chromatography (gradient of MeOH in DCM from 0% to 20%) followed by chiral phase chromatography (Phenomenex Lux Cellulose-1 150×21.2 mm 5 μm; gradient from 81% [heptane+0.1% DEA]−19% [iPrOH+0.1% DEA] to 45% [heptane+0.1% DEA]−55% [iPrOH+0.1% DEA]) afforded Intermediate 543A (297 mg, yield: 25%) and Intermediate 543B (314 g, yield: 27%) as clear oils.

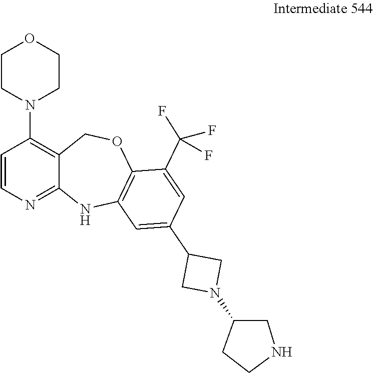 [SMILES: FC(F)(F)c1cc(C2CN([C@H]3CCNC3)C2)cc2c1OCc1c(N3CCOCC3)ccnc1N2]

(*S), pure isomer but absolute stereochemistry undetermined TFA (1.2 mL) was added to a solution of Intermediate 543B (314 mg, 0.55 mmol) in DCM (10 mL). The mixture was stirred overnight, then concentrated to dryness. The residue was washed with toluene twice and dried. The residue was dissolved in DCM/MeOH (9:1). Water was added and then solid Na₂CO₃ was added until pH=7. The layers were separated and the organic layer was dried over MgSO₄, filtered, and concentrated to afford Intermediate 544 as a foam (259 mg, yield: 99%).

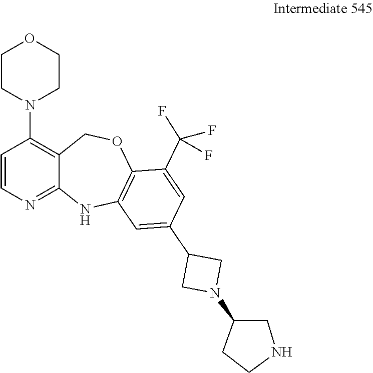 [SMILES: FC(F)(F)c1cc(C2CN([C@@H]3CCNC3)C2)cc2c1OCc1c(N3CCOCC3)ccnc1N2]

(*R), pure isomer but absolute stereochemistry undetermined Intermediate 545 was synthesized in a similar manner as Intermediate 544 using Intermediate 543A instead of Intermediate 543B.

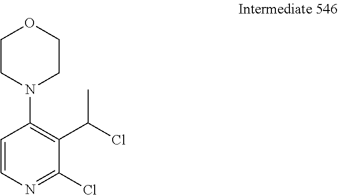 [SMILES: CC(Cl)c1c(N2CCOCC2)ccnc1Cl]

Thionyl chloride [CAS: 7719-09-7] (9.28 mL, 127.93 mmol) was slowly added to a solution of Intermediate 26 (20.7 g, 85.29 mmol) in DCM (300 mL) at 0° C. The reaction mixture was stirred at room temperature for 3 h. The reaction mixture was poured into a stirred mixture of water/ice/DCM. The layers were separated and the organic layer was washed with water, dried with MgSO₄, filtered, and concentrated to dryness to afford Intermediate 546 (19.3 g, yield: 87%).

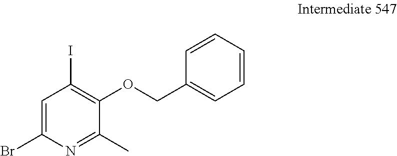 [SMILES: Cc1nc(Br)cc(I)c1OCc1ccccc1]

Benzyl bromide [CAS: 100-39-0] (29.2 mL, 245 mmol) was added to a solution of 6-bromo-4-iodo-2-methyl-3-pyridinol [CAS: 637348-80-2] (67 g, 213 mmol) and K₂CO₃ (44.25 g, 320 mmol) in DMF (200 mL) and the reaction mixture was stirred at room temperature overnight. The reaction mixture was diluted with EtOAc and washed with brine. The organic layer was dried with MgSO₄, filtered, and concentrated to dryness, then a mixture of EtOAc and heptane (1:1) was added. The resulting solution was concentrated under vacuum until a solid was formed. The solid was filtered, washed with heptane, and dried under high vacuum to afford Intermediate 547 (53 g, yield: 61%).

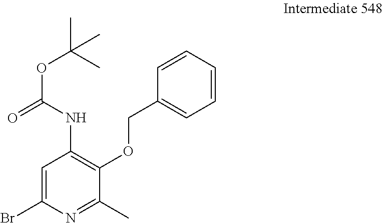 [SMILES: Cc1nc(Br)cc(NC(=O)OC(C)(C)C)c1OCc1ccccc1]

Intermediate 547 (34.7 g, 85.88 mmol), Cs₂CO₃ (55.96 g, 171.76 mmol), Xantphos (CAS: 161265-03-8) (2.98 g, 5.15 mmol), and Pd₂(dba)₃ [CAS: 51364-51-3] (2.36 g, 2.58 mmol) were suspended in toluene (400 mL) and stirred for 15 min. tert-Butyl carbamate (11.07 g, 94.47 mmol) was then added and the reaction mixture was stirred at 50° C. for 15 h. The reaction mixture was then cooled to room temperature, diluted with brine, and extracted with EtOAc. The combined organic layers were washed with brine, dried over MgSO₄, filtered, concentrated to dryness and purified over silica gel (gradient of EtOAc in heptane) to afford Intermediate 548 (32.9 g, yield: 82%).

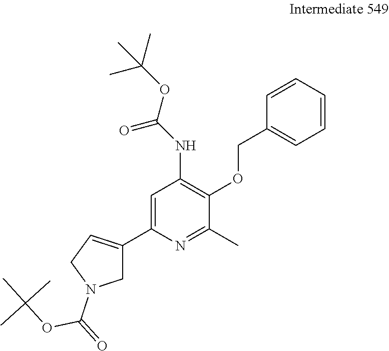 [SMILES: Cc1nc(C2=CCN(C(=O)OC(C)(C)C)C2)cc(NC(=O)OC(C)(C)C)c1OCc1ccccc1]

Pd(dppf)Cl₂·DCM [CAS: 95464-05-4] (1.67 g, 2.03 mmol) was added to a suspension of Intermediate 548 (16 g, 40.68 mmol), tert-butyl 3-(4,4,5,5-tetramethyl-1,3,2-dioxaborolan-2-yl)-2,5-dihydro-1H-pyrrole-1-carboxylate (CAS: 212127-83-8) (141.4 g, 48.82 mmol), and K₃PO₄ (17.27 g, 81.37 mmol) in 1,4-dioxane (144 mL) and water (24 mL). The mixture was stirred overnight at 80° C., then was partitioned between EtOAc and brine. The combined organic layers were dried over MgSO₄, filtered, concentrated to dryness, and purified over silica gel (heptane/EtOAc gradient) to give Intermediate 549 (14.9 g, yield: 76%).

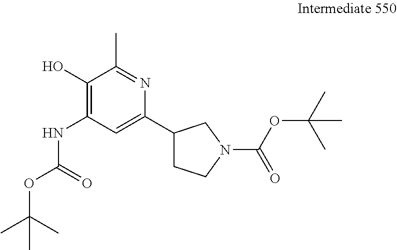 [SMILES: Cc1nc(C2CCN(C(=O)OC(C)(C)C)C2)cc(NC(=O)OC(C)(C)C)c1O]

Pd/C 10% (1 g) was added to a solution of Intermediate 549 (14.9 g, 30.94 mmol) in MeOH (200 mL) and THF (100 mL) and the mixture was hydrogenated at room temperature under H₂ atmosphere for 72 h. The mixture was filtered over celite and evaporated until dryness to yield Intermediate 550 (12.8 g, quant yield), used without further purification.

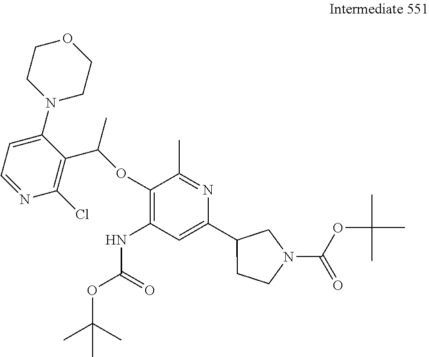 [SMILES: Cc1nc(C2CCN(C(=O)OC(C)(C)C)C2)cc(NC(=O)OC(C)(C)C)c1OC(C)c1c(N2CCOCC2)ccnc1Cl]

K₂CO₃ (8.92 g, 64.55 mmol) was added to a suspension of Intermediate 550 (12.7 g, 32.28 mmol) in DMF (50 mL). The reaction mixture was stirred at room temperature, then Intermediate 546 (8.43 g, 32.27 mmol) was added in small portions over 1 h. The mixture was stirred at room temperature for 16 h, then another portion of Intermediate 546 (4.21 g, 16.13 mmol) was added and the reaction mixture was stirred for another 72 h. To reach full conversion, another portion of Intermediate 546 (4.21 g, 16.13 mmol) was added and the reaction mixture was stirred for a further 16 h. The reaction mixture was diluted with EtOAc and washed with brine. The combined organic layers were dried over MgSO₄, filtered, concentrated to dryness, and purified over silica gel (heptane/EtOAc gradient) to give Intermediate 551 (18 g, yield: 90%).

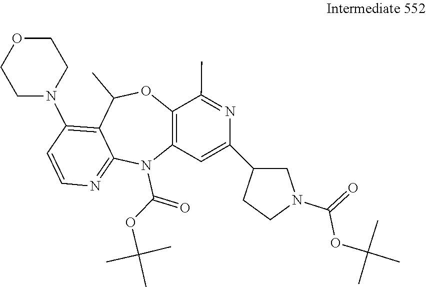 [SMILES: Cc1nc(C2CCN(C(=O)OC(C)(C)C)C2)cc2c1OC(C)c1c(N3CCOCC3)ccnc1N2C(=O)OC(C)(C)C]

Intermediate 551 (18 g, 29.12 mmol) and Cs₂CO₃ (18.97 g, 58.24 mmol, 2 eq.) were bubbled with nitrogen for 15 min in 1,4-dioxane (400 mL). Pd₂(dba)₃ [CAS: 51364-51-3] (2.67 g, 2.91 mmol, 0.1 eq.) and Xantphos (CAS: 161265-03-8) (3.37 g, 5.82 mmol, 0.2 eq.) were added. The resulting mixture was stirred at 100° C. overnight. To push the reaction to completion, 1,4-dioxane (100 mL), Pd₂(dba)₃ [CAS: 51364-51-3] (2.67 g, 2.91 mmol, 0.1 eq.), and Xantphos (CAS: 161265-03-8) (3.37 g, 5.82 mmol, 0.2 eq.) were added and the reaction mixture was stirred under nitrogen atmosphere at 100° C. for 4 days. The reaction mixture was then cooled to room temperature, partitioned between brine and EtOAc, and extracted with EtOAc. The combined organic layers were dried over MgSO₄, filtered, concentrated to dryness and purified over silica gel (gradient of EtOAc in heptane) to afford Intermediate 552 (10.7 g, yield: 64%).

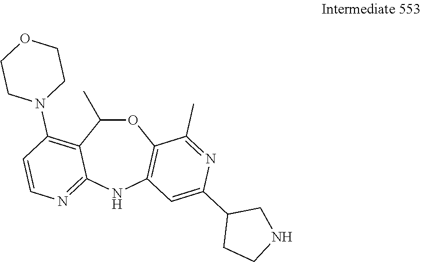 [SMILES: Cc1nc(C2CCNC2)cc2c1OC(C)c1c(N3CCOCC3)ccnc1N2]

TFA (15 mL) was added to a solution of Intermediate 552 (10.76 g, 18.5 mmol) in DCM (100 mL) and the mixture was stirred at room temperature for 2 h. The reaction mixture was concentrated to dryness and the residue was resuspended with DCM and poured into water/K₂CO₃ and extracted with DCM. The organic layer was washed with brine, dried over MgSO₄, filtered, and concentrated to dryness. The residue was dissolved in MeOH (200 mL) and K₂CO₃ (7.03 g, 50.89 mmol) was added. The reaction mixture was stirred for 2 h at room temperature, filtered, and concentrated to dryness to give Intermediate 553 (6.47 g, yield: 92%).

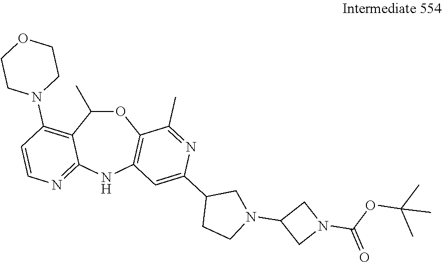 [SMILES: Cc1nc(C2CCN(C3CN(C(=O)OC(C)(C)C)C3)C2)cc2c1OC(C)c1c(N3CCOCC3)ccnc1N2]

AcOH (1.27 mL, 22.2 mmol) was added to a solution of Intermediate 553 (7.06 g, 18.5 mmol) and tert-butyl-3-oxoazetidine-1-carboxylate (CAS: 398489-26-4) (4.75 g, 27.75 mmol) in MeOH (200 mL). The reaction mixture was stirred for 2 h, before the addition of sodium cyanoborohydride [CAS: 25895-60-7] (1.74 g, 27.75 mmol) and the resulting mixture was stirred at room temperature for 18 h. Aqueous NaHCO₃ was then added and the mixture was extracted with EtOAc. The combined organic layers were washed with brine, dried over MgSO₄, filtered, concentrated to dryness, and purified over silica gel (gradient of EtOAc in heptane from 50% to 100%, then with a gradient of MeOH in DCM from 0 to 10%) to give Intermediate 554 (5.2 g, yield: 52%).

### Intermediate 555A, Intermediate 555B, Intermediate 555C, and Intermediate 555D

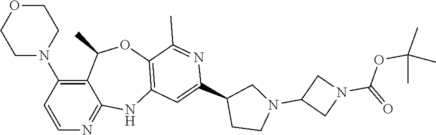 [SMILES: Cc1nc([C@@H]2CCN(C3CN(C(=O)OC(C)(C)C)C3)C2)cc2c1O[C@H](C)c1c(N3CCOCC3)ccnc1N2]

Intermediate 555A: (*R, *R) pure stereoisomer but absolute stereochemistry undetermined.

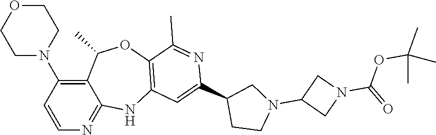 [SMILES: Cc1nc([C@@H]2CCN(C3CN(C(=O)OC(C)(C)C)C3)C2)cc2c1O[C@@H](C)c1c(N3CCOCC3)ccnc1N2]

Intermediate 555B: (*S, *R) pure stereoisomer but absolute stereochemistry undetermined

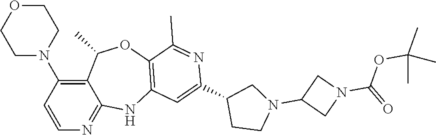 [SMILES: Cc1nc([C@H]2CCN(C3CN(C(=O)OC(C)(C)C)C3)C2)cc2c1O[C@@H](C)c1c(N3CCOCC3)ccnc1N2]

Intermediate 555C: (*S, *S) pure stereoisomer but absolute stereochemistry undetermined.

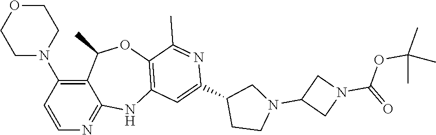 [SMILES: Cc1nc([C@H]2CCN(C3CN(C(=O)OC(C)(C)C)C3)C2)cc2c1O[C@H](C)c1c(N3CCOCC3)ccnc1N2]

Intermediate 555D: (*R, *S) pure stereoisomer but absolute stereochemistry undetermined.

The enantiomers of Intermediate 555 were separated by chiral SFC (CHIRALPAK AD-H 5 μm 250*30 mm, Mobile phase: 60% CO₂, 40% iPrOH (0.3% iPrNH₂)) to give Intermediate 555A (853 mg, yield: 17%), Intermediate 555B (798 mg, yield: 16%), Intermediate 555C (812 mg, yield: 16%) and Intermediate 555D (745 mg, yield: 15

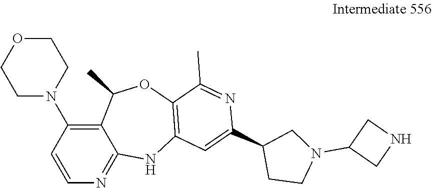 [SMILES: Cc1nc([C@@H]2CCN(C3CNC3)C2)cc2c1O[C@H](C)c1c(N3CCOCC3)ccnc1N2]

(*R, *R), pure stereoisomer but absolute stereochemistry undetermined.

Intermediate 555A (142 mg, 0.26 mmol) was dissolved in a mixture of TFA (0.8 mL) and DCM (1.2 mL) and the mixture was stirred for 2 h at room temperature. The reaction mixture was then concentrated to dryness to give Intermediate 556 (115 mg, quant. yield).

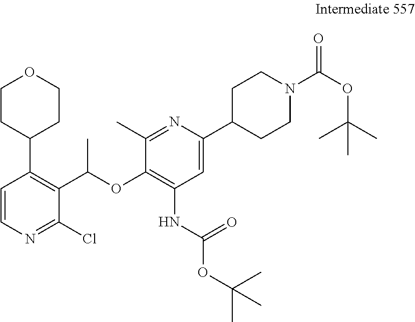 [SMILES: Cc1nc(C2CCN(C(=O)OC(C)(C)C)CC2)cc(NC(=O)OC(C)(C)C)c1OC(C)c1c(C2CCOCC2)ccnc1Cl]

DIAD (CAS: 2446-83-5) (1.32 mL, 6.72 mmol) was added to a solution of triphenylphosphine (1.76 g, 6.72 mmol) in THF (60 mL) at 0° C. under nitrogen atmosphere and the mixture was stirred for 10 min. Intermediate 480 (2.5 g, 10.34 mmol) was added at 0° C. and the mixture was stirred another 10 min before the addition of Intermediate 42 (2.1 g, 5.17 mmol). The reaction mixture was stirred at 85° C. for 20 h and then partitioned between EtOAc and water. The organic layer was dried over MgSO₄, filtered, concentrated to dryness, and purified via silica gel column chromatography (EtOAc in heptane from 0/100 to 100/0) to afford Intermediate 557 (1.0 g, yield: 29%).

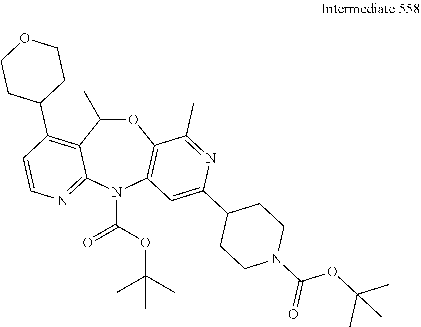 [SMILES: Cc1nc(C2CCN(C(=O)OC(C)(C)C)CC2)cc2c1OC(C)c1c(C3CCOCC3)ccnc1N2C(=O)OC(C)(C)C]

Intermediate 558 (663 mg, yield: 80%) was synthesized in a similar manner as Intermediate 531 using Intermediate 557 (1.0 g, 1.28 mmol) instead of Intermediate 530 and N-Boc-piperidin-4-one instead of N-Boc-azetidin-3-one.

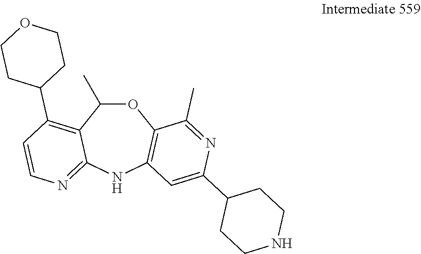 [SMILES: Cc1nc(C2CCNCC2)cc2c1OC(C)c1c(C3CCOCC3)ccnc1N2]

Intermediate 559 (884 mg, yield: 96%) was synthesized in a similar manner as Intermediate 532 using Intermediate 558 (663 mg, 1.03 mmol) instead of Intermediate 531.

### Intermediate 560A and Intermediate 560B

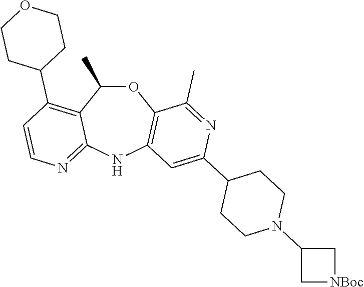 [SMILES: Cc1nc(C2CCN(C3CN(C(=O)OC(C)(C)C)C3)CC2)cc2c1O[C@H](C)c1c(C3CCOCC3)ccnc1N2] [0900] Intermediate 560A: (*R), pure stereoisomer but absolute stereochemistry undetermined.

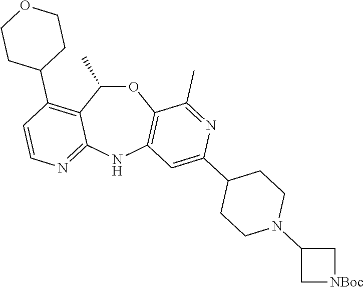 [SMILES: Cc1nc(C2CCN(C3CN(C(=O)OC(C)(C)C)C3)CC2)cc2c1O[C@@H](C)c1c(C3CCOCC3)ccnc1N2]

Intermediate 560 B: (*S), pure stereoisomer but absolute stereochemistry undetermined.

Sodium triacetoxyborohydride (165 mg, 0.778 mmol, 0.75 eq.) was added to a solution of Intermediate 559 (884 mg, 1.04 mmol), Et₃N (0.43 mL, 3.12 mmol) and 1-Boc-3-azetidinone (CAS [398489-26-4], 178 mg, 1.04 mmol, 1 eq.) in DCE (25 mL) at room temperature. The reaction mixture was stirred for 24 h. Stirring at room temperature was continued for 3 days with an addition of Boc-3-azetidinone (178 mg, 1.04 mmol, 1 eq.) and sodium triacetoxyborohydride (165 mg, 0.778 mmol, 0.75 eq.) every 24 h. Aqueous Na₂CO₃ (1 M) was added, the reaction mixture was extracted with DCM, and the organic layer was dried over MgSO₄, filtered, concentrated to dryness, and purified via silica gel column chromatography (gradient of DCM/MeOH (9:1) in DCM from 0% to 20%) followed by chiral SFC (Stationary phase: CHIRACEL OJ-H 5 μm 250*30 mm, Mobile phase: 85% CO₂, 15% MeOH (0.3% iPrNH₂)) to afford Intermediate 560A (79 mg, yield: 14%) and Intermediate 560B (61 mg, yield: 11%) as solids.

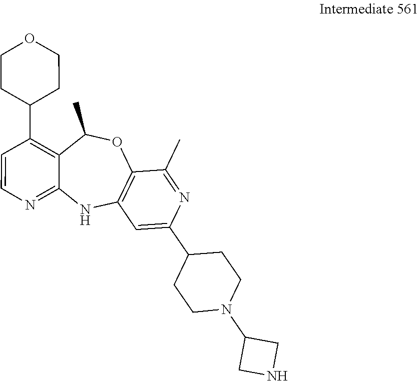 [SMILES: Cc1nc(C2CCN(C3CNC3)CC2)cc2c1O[C@H](C)c1c(C3CCOCC3)ccnc1N2]

(*R), pure stereoisomer but absolute stereochemistry undetermined. TFA (0.11 mL, 1.44 mmol) was added to a solution of Intermediate 560A (79 mg, 0.14 mmol) in DCM (10 mL) and the mixture was stirred at room temperature overnight. The reaction mixture was washed with aqueous Na₂CO₃ (1 M) and the organic layer was dried over MgSO₄, filtered, and concentrated to dryness to give Intermediate 561 as a beige solid (56 mg, yield: 86%).

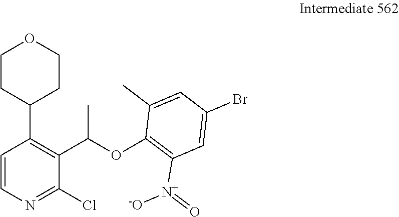

DIAD (2.28 mL, 11.58 mmol) was added to a solution of triphenylphosphine (3.04 g, 11.58 mmol) in THF (50 mL) at 0° C. and the reaction mixture was stirred for 10 min. Intermediate 480 (1.4 g, 5.79 mmol) was added at 0° C. and the reaction mixture was stirred for another 10 min. Finally, 4-bromo-2-methyl-6-nitrophenol (CAS: 20294-50-2) (2.69 g, 11.58 mmol) was added and the mixture was stirred at 85° C. for 20 h. The reaction mixture was partitioned between EtOAc and water. The organic layer was dried over MgSO₄, filtered, concentrated to dryness, and purified via silica gel column chromatography (EtOAc in heptane from 0/100 to 40/60) to give Intermediate 562 (2.67 g, quantitative yield).

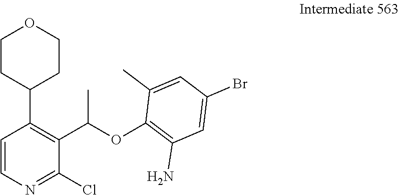 [SMILES: Cc1cc(Br)cc(N)c1OC(C)c1c(C2CCOCC2)ccnc1Cl]

Iron powder (3.27 g, 58.5 mmol) was added to a mixture of Intermediate 562 (2.67 g, 5.85 mmol) in AcOH (6.7 mL, 117 mmol, 20 eq.) and MeOH (55 mL). The reaction mixture was stirred at room temperature for 6 h. DCM and saturated aqueous NaHCO₃ were added and the mixure was filtered through a short pad of celite. The layers were separated. The organic layer was dried over MgSO₄, filtered, and concentrated to dryness. Purification by column chromatography over silica gel (gradient of EtOAc in heptane from 0% to 100%) afforded Intermediate 563 (2.38 g, yield: 95%).

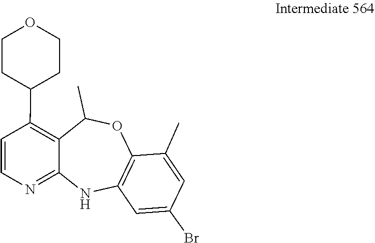 [SMILES: Cc1cc(Br)cc2c1OC(C)c1c(C3CCOCC3)ccnc1N2]

TFA (1.28 mL, 16.75 mmol) was added to a solution of Intermediate 564 (2.38 g, 5.58 mmol) in 1,4-dioxane (25 mL) and the reaction mixture was stirred at 120° C. for 8 h. The reaction mixture was diluted with EtOAc and washed with aqueous Na₂CO₃ (1 M). The organic layer was separated, dried over MgSO₄, filtered, and the solvents evaporated in vacuo. Purification by flash column chromatography on silica (EtOAc in heptane 0/100 to 100/0) afforded Intermediate 564 (1.94 g, yield: 89%).

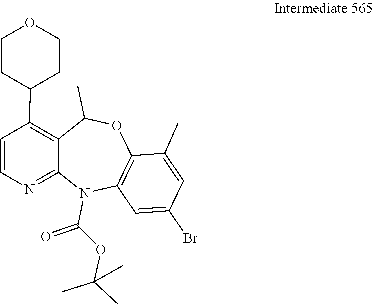 [SMILES: Cc1cc(Br)cc2c1OC(C)c1c(C3CCOCC3)ccnc1N2C(=O)OC(C)(C)C]

Boc anhydride (2.29 mL, 9.96 mmol) was added to a stirred solution of Intermediate 564 (1.94 g, 4.98 mmol) and DMAP (60 mg, 0.49 mmol) in DCE (45 mL) and the mixture was stirred at reflux for 2 h. The solvent was evaporated in vacuo. Purification by flash column chromatography (silica; EtOAc in heptane 0/100 to 100/0) afforded Intermediate 565 (1.6 g, yield: 59%).

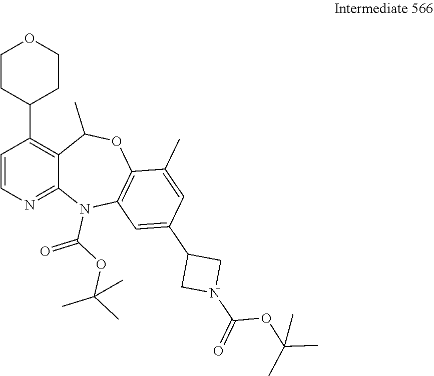 [SMILES: Cc1cc(C2CN(C(=O)OC(C)(C)C)C2)cc2c1OC(C)c1c(C3CCOCC3)ccnc1N2C(=O)OC(C)(C)C]

Intermediate 565 (1.6 g, 3.28 mmol) was dissolved in DMA (10 mL) and the solution was bubbled with nitrogen for 5 min, then Pd(dppf)C12.DCM (0.13 g, 0.16 mmol, 0.05 eq.) and CuI (62 mg, 0.33 mmol, 0.1 eq.) were added. [1-[(1,1-Dimethylethoxy)carbonyl]-3-azetidinyl]iodozine [CAS: 206446-38-0] (0.31 M solution in DMA, 21.1 mL, 6.56 mmol, 2 eq.) was added and the resulting mixture was heated at 80° C. overnight. The mixture was allowed to cool to room temperature and diluted with EtOAc. Water and aqueous NH₄Cl were then added. The organic layer was separated, washed with brine, dried over MgSO₄, filtered, and concentrated to dryness. Chromatography over silica gel (EtOAc in heptane from 0 to 50%) afforded Intermediate 566 as a brown solid (970 mg, yield: 52%).

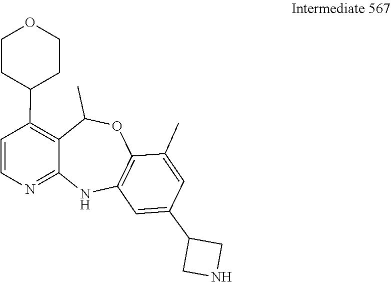 [SMILES: Cc1cc(C2CNC2)cc2c1OC(C)c1c(C3CCOCC3)ccnc1N2]

TFA (3.35 mL, 43.56 mmol) was added to solution of Intermediate 566 (1.2 g, 2.18 mmol) in DCM (45 mL). The mixture was stirred overnight and concentrated in vacuo. The residue was dissolved in DCM and washed with a mixture of aqueous Na₂CO₃ (1 M) and brine. The organic layer was dried over MgSO₄, filtered, and concentrated to dryness to afford Intermediate 567 (1.79 g, 85%).

### Intermediate 568A and Intermediate 568B

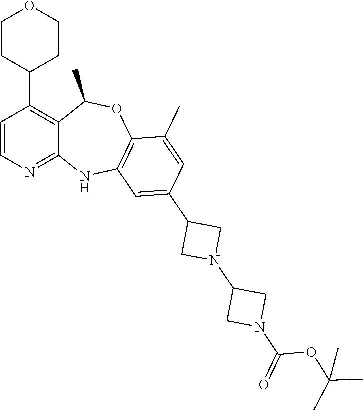 [SMILES: Cc1cc(C2CN(C3CN(C(=O)OC(C)(C)C)C3)C2)cc2c1O[C@H](C)c1c(C3CCOCC3)ccnc1N2] [0911] Intermediate 568A: (*R), pure stereoisomer but absolute stereochemistry undetermined.

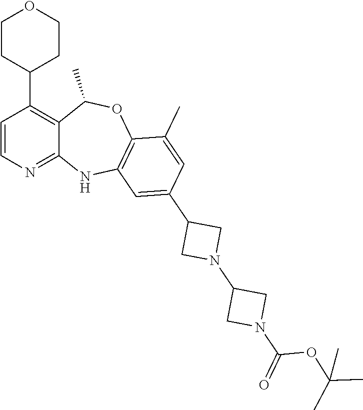 [SMILES: Cc1cc(C2CN(C3CN(C(=O)OC(C)(C)C)C3)C2)cc2c1O[C@@H](C)c1c(C3CCOCC3)ccnc1N2]

Intermediate 568B: (*S), pure stereoisomer but absolute stereochemistry undetermined.

Sodium triacetoxyborohydride (1.45 g, 6.83 mmol) was added to a solution of Intermediate 567 (1.79 g, 2.28 mmol), triethylamine (1.27 mL, 9.1 mmol), and 1-Boc-3-azetidinone (CAS: 398489-26-4) (1.17 g, 6.83 mmol) in DCE (30 mL). The mixture was stirred at room temperature overnight. Aqueous Na₂CO₃ (1 M) was added, the reaction mixture was extracted with DCM, the organic layer was dried over MgSO₄, filtered, concentrated to dryness and purified via silica gel column chromatography (gradient of DCM/MeOH (9:1) in DCM from 0% to 30%), followed by chiral phase chromatography (Column: Lux-Amylose-2; gradient: 75% [n-heptane+0.1% DEA]−25% [iPrOH+0.1% DEA] to 0% [n-heptane+0.1% DEA]−100% [iPrOH+0.1% DEA]) to afford Intermediate 568A (108 mg, yield: 9%) and Intermediate 568B (104 mg, yield: 9%).

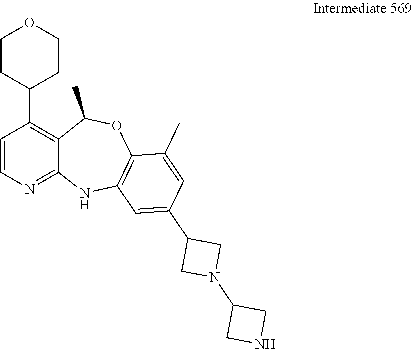 [SMILES: Cc1cc(C2CN(C3CNC3)C2)cc2c1O[C@H](C)c1c(C3CCOCC3)ccnc1N2]

(*R), pure stereoisomer but absolute stereochemistry undetermined. Intermediate 569 (87 mg, yield: 92%) was synthesized in a similar manner as Intermediate 561 using Intermediate 568A (108 mg, 0.21 mmol) instead of Intermediate 560A.

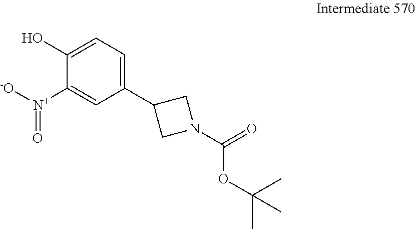

tert-Butyl 3-(4-hydroxyphenyl)azetidine-1-carboxylate (CAS: 1782327-13-2) (45 g, 180.5 mmol) was suspended in AcOH (700 mL) and nitric acid (13.4 mL, 198.55 mmol) was added dropwise at 40° C. over 20 min. The mixture was diluted with EtOAc and washed with a solution of saturated aqueous Na₂CO₃, water, and brine. The organic layer was dried over MgSO₄, filtered, concentrated to dryness, and the residue was suspended in MTBE. The mixture was stirred for 5 min and filtered. The filter cake was rinsed with MTBE and dried under high vacuum to give a first batch of Intermediate 570 as a yellow solid. The filtrate was concentrated under vacuum and the residue was purified by flash column chromatography (eluent: EtOAc/petroleum ether from 0/100 to 18/82) to give a second batch of Intermediate 570 (combined batches: 39 g, combined yield: 73%) as a yellow solid.

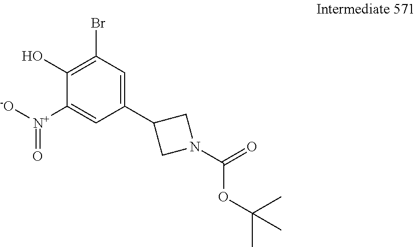

NBS (20.38 g, 114.52 mmol) was added portionwise to a suspension of Intermediate 570 (34 g, 114.52 mmol) and silica gel (210 g) in DCM (1.1 L) at −15° C. The mixture was stirred at −15° C. for 30 min and then filtered. The filter cake was rinsed with DCM, the filtrate was concentrated, and the residue was purified by flash column chromatography over silica gel (eluent: EtOAc/petroleum ether from 0/100 to 18/82, gradient) to give Intermediate 571 (yield: 69%).

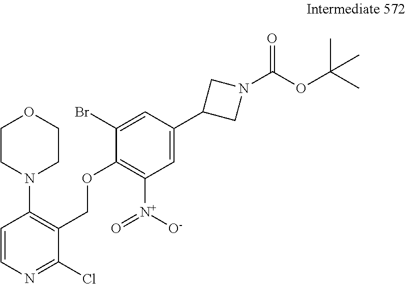 [SMILES: CC(C)(C)OC(=O)N1CC(c2cc(Br)c(OCc3c(N4CCOCC4)ccnc3Cl)c([N+](=O)[O-])c2)C1]

DIAD (CAS [2446-83-5], 2.1 mL, 10.67 mmol) was added to a mixture of Intermediate 571 (3.07 g, 8.226 mmol), Intermediate 59 (2 g, 8.746 mmol), and triphenylphosphine (2.8 g, 10.71 mmol) in THF (100 mL) at 0° C. The reaction mixture was stirred at room temperature for 18 h. The reaction mixture was poured into water and extracted with EtOAc. The organic layer was separated, dried over MgSO₄, filtered, concentrated to dryness, and purified by column chromatography (Irregular SiOH 15-40 μm 80 g GraceResolv®, heptane/EtOAc from 80/20 to 40/60) to afford Intermediate 572 (4.0 g, yield: 83%).

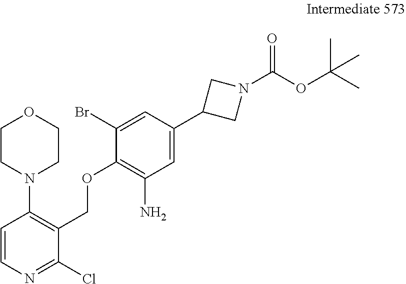 [SMILES: CC(C)(C)OC(=O)N1CC(c2cc(N)c(OCc3c(N4CCOCC4)ccnc3Cl)c(Br)c2)C1]

Iron (3.85 g, 68.938 mmol) was added to a mixture of Intermediate 572 (4 g, 6.851 mmol) and AcOH (8 mL, 139.74 mmol) in MeOH (60 mL) and the reaction mixture was stirred at room temperature overnight. DCM and an aqueous saturated solution of NaHCO₃ were added and the mixture was filtered through a short pad of celite. The organic layer was separated, dried over MgSO₄, filtered, and concentrated to dryness to afford Intermediate 573 (3.74 g, yield: 99%).

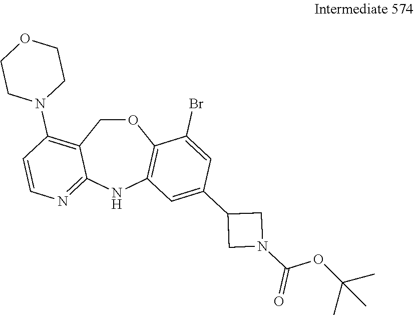 [SMILES: CC(C)(C)OC(=O)N1CC(c2cc(Br)c3c(c2)Nc2nccc(N4CCOCC4)c2CO3)C1]

A mixture of Intermediate 573 (3.74 g, 6.752 mmol) in t-amyl alcohol (26 mL) was stirred at 120° C. for 2 h. The reaction mixture was cooled down to room temperature and concentrated to dryness. Purification by column chromatography (Irregular SiOH 15-40 μm 80 g GraceResolv, Gradient from 98% DCM, 2% MeOH, 0.2% NH₄OH to 92% DCM, 8% MeOH, 0.8% NH₄OH) afforded Intermediate 574 (2.28 g, yield: 65

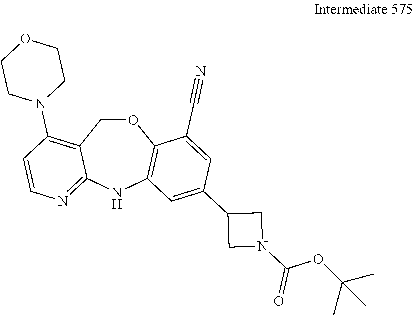 [SMILES: CC(C)(C)OC(=O)N1CC(c2cc(C#N)c3c(c2)Nc2nccc(N4CCOCC4)c2CO3)C1]

A mixture of Intermediate 574 (0.8 g, 1.546 mmol), zinc cyanide (0.84 g, 7.11 mmol), dppf [CAS: 12150-46-8] (0.087 g, 0.160 mmol), and tris(dibenzylideneacetone)dipalladium(0) [CAS: 52409-22-0] (0.085 g, 0.09 mmol) in DMF (8.5 mL) was stirred at 140° C. in a sealed tube under microwave irradiation for 40 min. The reaction mixture was cooled down to room temperature, poured into a mixture of EtOAc and water and it was stirred for 10 min. The organic layer was separated, dried over MgSO₄, filtered, concentrated to dryness and purified by column chromatography (Irregular SiOH 15-40 μm 40 g GraceResolv®, mobile phase Gradient from 99% DCM, 1% MeOH, 0.1% NH₄OH to 94% DCM, 6% MeOH, 0.6% NH₄OH) to afford Intermediate 575 (583 mg, yield: 81%).

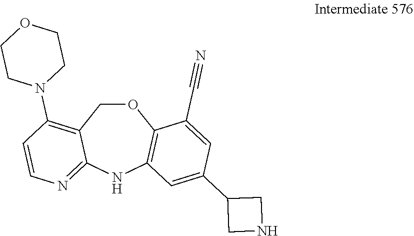 [SMILES: N#Cc1cc(C2CNC2)cc2c1OCc1c(N3CCOCC3)ccnc1N2]

HCl (4 M in 1,4-dioxane, 2.7 mL, 10.8 mmol) was added slowly to a mixture of Intermediate 575 (535 mg, 1.154 mmol) in 1,4-dioxane (6 mL) at 0° C., then MeOH (1.5 mL) was added and the reaction mixture was stirred at room temperature for 4 h. The solvent was evaporated until dryness to give Intermediate 576 (790 mg, quantitative yield).

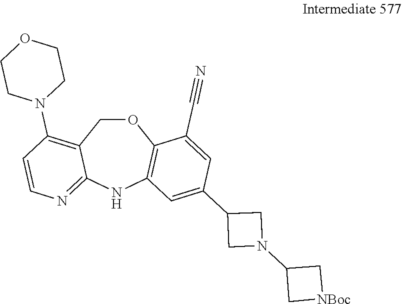 [SMILES: CC(C)(C)OC(=O)N1CC(N2CC(c3cc(C#N)c4c(c3)Nc3nccc(N5CCOCC5)c3CO4)C2)C1]

A mixture of Intermediate 576 (503 mg, 1.15 mmol), Boc azetidinone [CAS: 398489-26-4] (296 mg, 1.73 mmol), AcOH (0.12 mL), and sodium triacetoxyborohydride (488 mg, 2.303 mmol) in DCM (5 mL) was stirred at room temperature for 2 days. The reaction mixture was poured into water and extracted with DCM. The organic layer was separated, dried over MgSO₄, filtered, concentrated to dryness, and purified by column chromatography (Irregular SiOH 15-40 μm 40 g GraceResolv®, gradient from 98% DCM, 2% MeOH, 0.2% NH₄OH to 92% DCM, 8% MeOH, 0.8% NH₄OH) to afford Intermediate 577 (300 mg, yield: 50%).

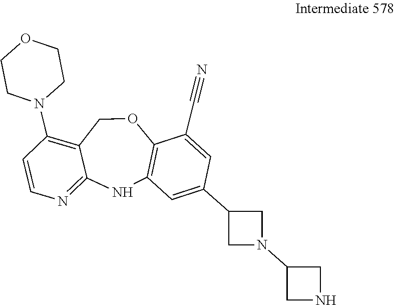 [SMILES: N#Cc1cc(C2CN(C3CNC3)C2)cc2c1OCc1c(N3CCOCC3)ccnc1N2]

HCl (4 M in 1,4-dioxane, 1.5 mL, 6 mmol) was added to a solution of Intermediate 577 (300 mg, 0.578 mmol) in 1,4-dioxane (5 mL) at 0° C. The reaction mixture was stirred for 5 h, then the solvent was evaporated until dryness to give Intermediate 578 (310 mg, quantitative yield).

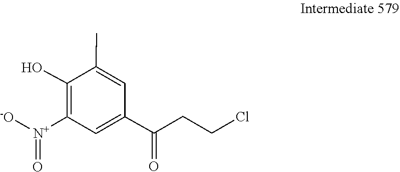

Nitric acid (65%, 4.38 mL, 63.88 mmol) was added dropwise to a solution of 3-chloro-1-(4-hydroxy-3-methyl-phenyl)-propan-1-one (CAS: 7182-40-3) (8.46 g, 42.59 mmol) in AcOH (80 mL) at room temperature and the reaction mixture was stirred for 30 min. The mixture was diluted with diethyl ether and washed with water and brine. The etheral solution was dried over MgSO₄, filtered, and concentrated to dryness. Purification by chromatography over silica gel (gradient of EtOAc in heptane from 0 to 20%) afforded Intermediate 579 (9.16 g, 80% purity, yield: 71%).

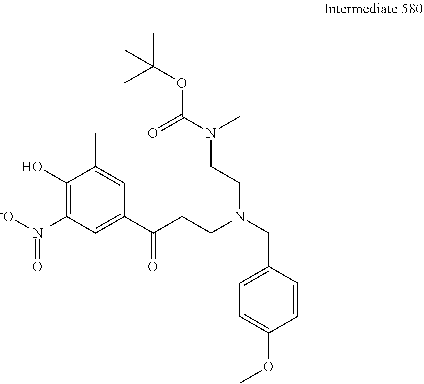

Intermediate 579 (4.73 g, 19.43 mmol), carbamic acid, N-[2-[[(4-methoxyphenyl)methyl]amino]ethyl]-N-methyl-, 1,1-dimethylethyl ester (CAS: 1834353-48-8) (5.72 g, 19.43 mmol), and K₂CO₃ (5.37 g, 38.86 mmol) were stirred in DMF (50 mL) at room temperature for 2 days. The reaction mixture was diluted with water and aqueous KHSO₄ (1 M) was added until pH=7. The reaction mixture was extracted with DCM and the organic layer was dried over MgSO₄, filtered, and concentrated to dryness. Purification by silica gel chromatography (gradient of EtOAc in heptane from 0 to 50%) afforded Intermediate 580 (6.1 g, yield: 50%).

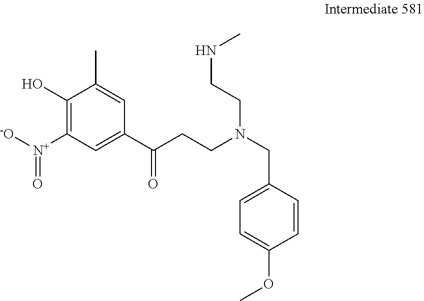

TFA (10.4 mL, 136 mmol) was added to a solution of Intermediate 580 (1.29 g, 2.57 mmol) in DCM (60 mL) and the solution was stirred for 30 min at room temperature. The reaction mixture was concentrated to dryness. The residue was suspended in toluene and mixture was concentrated under reduced pressure to the give Intermediate 581 (1.97 g, quantitative yield).

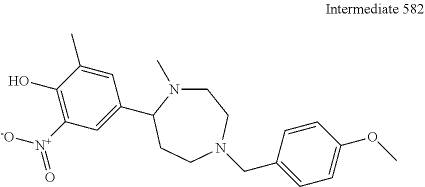

Intermediate 581 (1.62 g, 2.57 mmol) in DCE (50 mL) was treated with sodium triacetoxyborohydride [CAS: 56553-60-7] (1.09 g, 5.14 mmol) at room temperature for 1 h. The reaction mixture was diluted with DCM and treated with aqueous Na₂CO₃ (1 M). Aqueous NaOH (1 M) was also added. After 30 min stirring, the pH was brought to 7 with aqueous HCl (6 M). The organics were then extracted with DCM, dried over MgSO₄, filtered, and concentrated to dryness. Flash chromatography over silica gel (gradient of MeOH in DCM from 0 to 5%) afforded Intermediate 582 (657 mg, yield: 63%).

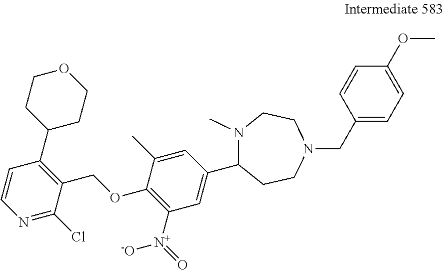

Intermediate 408 (0.39 g, 1.7 mmol) and triphenylphosphine (0.58 g, 2.21 mmol) were added to a solution of Intermediate 582 (0.66 g, 1.7 mmol) in THF (20 mL) under nitrogen atmosphere. A solution of DIAD [CAS: 2446-83-5] (0.43 mL, 2.21 mmol) in THF (5 mL) was added dropwise at room temperature. The mixture was stirred for 30 min and then concentrated to dryness. Chromatography over silica gel (gradient of MeOH in DCM from 0 to 5%) afforded Intermediate 583 (900 mg, yield: 85%) as a yellow amorphous solid.

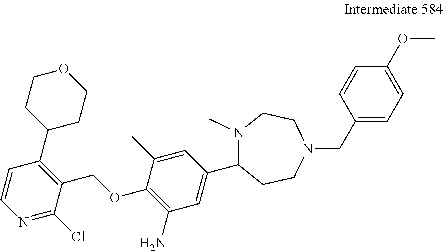 [SMILES: COc1ccc(CN2CCC(c3cc(C)c(OCc4c(C5CCOCC5)ccnc4Cl)c(N)c3)N(C)CC2)cc1]

Iron [CAS: 7439-89-6] (0.84 g, 15.12 mmol) was added to a vigorously stirred solution of Intermediate 583 (900 mg, 1.51 mmol) in AcOH (1.75 mL) and MeOH (20 mL) at room temperature and the reaction mixture was stirred for 7 h. The mixture was filtered and was then diluted with water and DCM. Solid Na₂CO₃ was added until pH>7. The solids were filtered through a pad of Celite, and the organic layer was separated, dried over MgSO₄, filtered, and concentrated to dryness to afford Intermediate 584 (874 mg, quantitative yield).

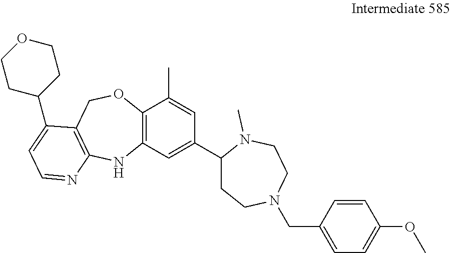 [SMILES: COc1ccc(CN2CCC(c3cc(C)c4c(c3)Nc3nccc(C5CCOCC5)c3CO4)N(C)CC2)cc1]

A solution of Intermediate 584 (0.87 g, 1.54 mmol) and Cs₂CO₃ (0.75 mg, 2.31 mmol) in 1,4-dioxane (15 mL) was degassed by bubbling nitrogen for 15 min. Pd₂(dba)₃ (CAS [51364-51-3], 71 mg, 0.077 mmol) and Xantphos (CAS: 161265-03-8) (89 mg, 0.15 mmol) were added. The resulting mixture was stirred at reflux overnight. The reaction mixture was then cooled to room temperature, diluted with H₂O, and extracted with DCM. The combined organic layers were dried over MgSO₄, filtered, concentrated to dryness, and purified over silica gel (gradient of MeOH in DCM from 0 to 10%) to afford Intermediate 585 (492 mg, yield: 60%).

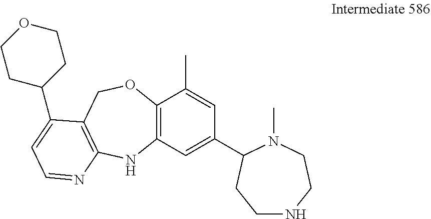 [SMILES: Cc1cc(C2CCNCCN2C)cc2c1OCc1c(C3CCOCC3)ccnc1N2]

Pd/C (10%, 380 mg) was added to an ice-cold solution of Intermediate 585 (490 mg, 0.93 mmol) in MeOH (20 mL), and the reaction mixture was hydrogenated at room temperature under H₂ atmosphere for 20 h. AcOH (2 mL) was then added and the reaction mixture was stirred for a further 24 h at room temperature. The mixture was filtered over celite and evaporated until dryness to yield Intermediate 586 (178 mg, yield: 45%).

### Intermediate 587A and Intermediate 587B

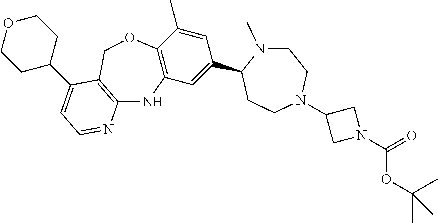 [SMILES: Cc1cc([C@@H]2CCN(C3CN(C(=O)OC(C)(C)C)C3)CCN2C)cc2c1OCc1c(C3CCOCC3)ccnc1N2]

Intermediate 587A: (*S) pure stereoisomer but absolute stereochemistry undetermined.

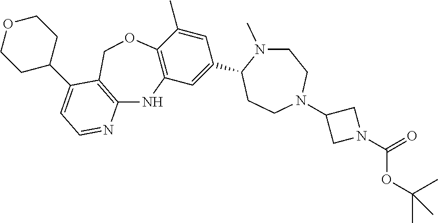 [SMILES: Cc1cc([C@H]2CCN(C3CN(C(=O)OC(C)(C)C)C3)CCN2C)cc2c1OCc1c(C3CCOCC3)ccnc1N2]

Intermediate 587B: (*R) pure stereoisomer but absolute stereochemistry undetermined.

Intermediate 586 (0.51 g, 1.26 mmol), AcOH (0.072 mL, 1.26 mmol), and 1-Boc-3-azetidinone (CAS: 398489-26-4) (430 mg, 2.51 mmol) were stirred in DCE (20 mL) for 30 min, then sodium triacetoxyborohydride (319 mg, 1.51 mmol) was added and the mixture was stirred for 4 h at room temperature. Aqueous Na₂CO₃ (1 M) was added, the reaction mixture was extracted with DCM, the organic layer was dried over MgSO₄, filtered, and concentrated to dryness. Purification by silica gel column chromatography (gradient of MeOH in DCM from 0% to 5%) followed by chiral chromatography (Column: Lux CELLULOSE-1 (150×21.2 mm); gradient of ACN (0.1% DEA) in iPrOH (0.1% DEA) from 5 to 70%) afforded Intermediate 587A (168 mg, yield: 23%) and Intermediate 587B (229 mg, yield: 32%).

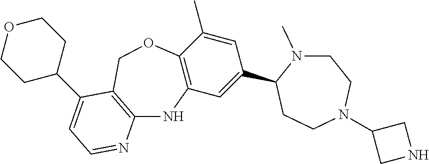 [SMILES: Cc1cc([C@@H]2CCN(C3CNC3)CCN2C)cc2c1OCc1c(C3CCOCC3)ccnc1N2]

(*S) pure stereoisomer but absolute stereochemistry undetermined. TFA (0.6 mL, 7.9 mmol) was added to solution of Intermediate 587A (168 mg, 0.3 mmol) in DCM (10 mL) at 0° C. The mixture was stirred overnight at room temperature and then concentrated in vacuo. The residue was taken up in DCM and washed with aqueous Na₂CO₃ (1 M). The aqueous layer was extracted with DCM and the combined organic layer was dried over MgSO₄, filtered, and concentrated to dryness to afford Intermediate 588 (135 mg, 92%).

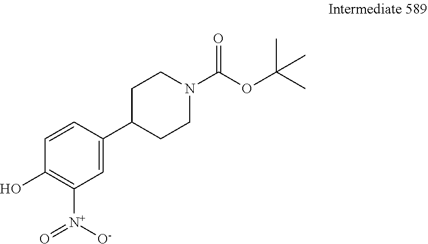 [SMILES: CC(C)(C)OC(=O)N1CCC(c2ccc(O)c([N+](=O)[O-])c2)CC1]

Nitric acid (65%, 2.67 mL, 39.66 mmol) was added dropwise to a solution of 1-piperidinecarboxylic acid, 4-(4-hydroxyphenyl)-, 1,1-dimethylethyl ester [CAS: 149377-19-5] (10 g, 36.05 mmol) in AcOH (100 mL) at 40° C. The mixture was stirred at 40° C. for 10 min, then the mixture was poured into a mixture of ice-water and EtOAc. The separated aqueous layer was extracted with EtOAc and the combined organic layer was neutralized with aqueous NaHCO₃ and stirred for 20 min. The layers were separated and the organic layer was washed with brine, dried over MgSO₄, filtered, and concentrated to dryness. Purification by flash column chromatography over silica gel (eluent: EtOAc/petroleum ether from 0/100 to 15/85, gradient) afforded Intermediate 589 (8.0 g, yield: 69%).

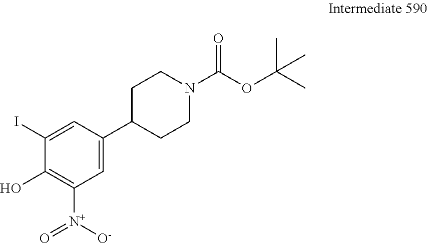 [SMILES: CC(C)(C)OC(=O)N1CCC(c2cc(I)c(O)c([N+](=O)[O-])c2)CC1]

NIS [CAS: 516-12-1] (4.98 g, 22.13 mmol) was added in one portion to a stirred solution of Intermediate 589 (4.76 g, 14.77 mmol) in CHCl₃ (69 mL) at room temperature under a nitrogen atmosphere. The reaction mixture was stirred at room temperature for 16 h, then more NIS (0.5 g, 2.22 mmol) was added and the mixture was stirred at room temperature for a further 16 h. The reaction mixture was quenched with saturated aqueouw Na₂S₂O₃, extracted with EtOAc, and the organic layer was washed with brine, dried over Na₂SO₄, filtered, and concentrated in vacuo. The residue was purified by flash column chromatography (silica; DCM 100% isocratic) to afford Intermediate 590 (3.64 g, yield: 55%).

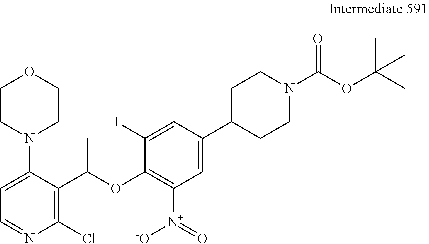

DIAD (CAS [2446-83-5], 3.2 mL, 16.24 mmol) was added dropwise to a stirred solution of Intermediate 590 (3.64 g, 8.12 mmol), Intermediate 26 (1.97 g, 8.12 mmol), and triphenylphosphine (4.26 g, 16.24 mmol) in THF (39 mL) at room temperature and the reaction mixture was stirred for 18 h. The reaction mixture was concentrated to dryness and purified by flash column chromatography (silica; EtOAc in heptane 20/80 to 50/50) to afford Intermediate 591 (6.7 g, quantitative yield).

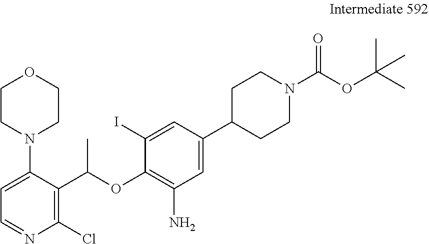 [SMILES: CC(Oc1c(N)cc(C2CCN(C(=O)OC(C)(C)C)CC2)cc1I)c1c(N2CCOCC2)ccnc1Cl]

A solution of Intermediate 591 (3.43 g, 5.1 mmol) was hydrogenated in an H-CUBE using Pt/C as catalyst (1 mL/min, CatCart 70 mm Pt/C 10% cartridge, Full-H2 mode, 80° C., 2 cycles). The reaction mixture was concentrated in vacuo and the residue was purified by flash column chromatography (silica; MeOH in DCM 0/100 to 5/95) to afford Intermediate 592 (970 mg, yield: 30%).

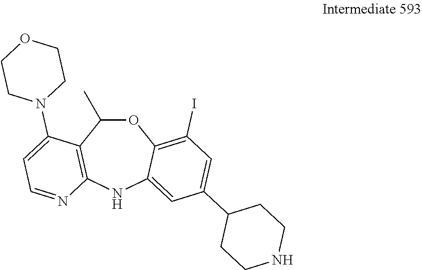 [SMILES: CC1Oc2c(I)cc(C3CCNCC3)cc2Nc2nccc(N3CCOCC3)c21]

TFA (0.32 mL, 4.22 mmol) was added to a solution of Intermediate 592 (970 mg, 1.06 mmol) in 1,4-dioxane (8.25 mL) in a sealed tube at room temperature. The mixture was stirred at 90° C. for 48 h. The mixture was then concentrated in vacuo and the residue was purified by flash column chromatography (silica; 10% of 7 N solution of ammonia in MeOH in DCM/DCM 0/100 to 100/00) to afford Intermediate 593 (506 mg, yield: 70%).

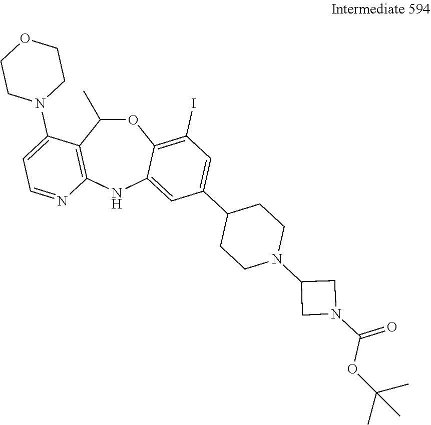 [SMILES: CC1Oc2c(I)cc(C3CCN(C4CN(C(=O)OC(C)(C)C)C4)CC3)cc2Nc2nccc(N3CCOCC3)c21]

Sodium triacetoxyborohydride (430 mg, 2.03 mmol) was added to a mixture of Intermediate 593 (506 mg, 1.0 mmol), AcOH (0.097 mL, 1.7 mmol), and 1-Boc-3-azetidinone (CAS: 398489-26-4) (262 mg, 1.53 mmol) in DCM (5.3 mL). The mixture was stirred for 16 h at room temperature. Saturated aqueous NaHCO₃ was added, the reaction mixture was then extracted with DCM, and the combined organic layer was dried over MgSO₄, filtered, and concentrated to dryness. Purification via silica gel column chromatography (gradient of MeOH in DCM from 0% to 7%) afforded Intermediate 594 (694 mg, 82% purity, yield: 86%).

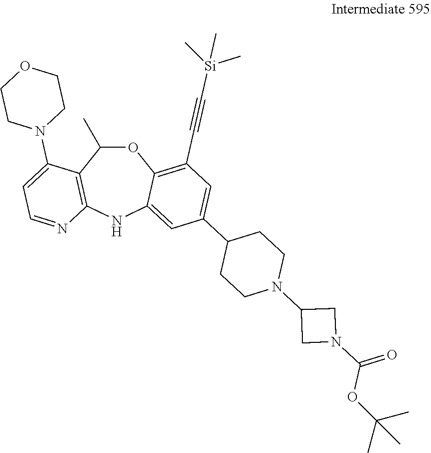 [SMILES: CC1Oc2c(C#C[Si](C)(C)C)cc(C3CCN(C4CN(C(=O)OC(C)(C)C)C4)CC3)cc2Nc2nccc(N3CCOCC3)c21]

Et₃N (0.81 mL, 5.84 mmol) was added to a stirred suspension of Intermediate 594 (644 mg, 0.97 mmol), trimethylsilylacetylene [CAS: 1066-54-2] (0.41 mL, 2.92 mmol), PdCl₂(PPh₃)₂ (71 mg, 0.1 mmol), and CuI (10.3 mg, 0.054 mmol) in DMF (12.9 mL) under a nitrogen atmosphere. The mixture was stirred at room temperature for 90 min, then diluted with saturated aqueous NH₄Cl, and extracted with EtOAc. The organic layer was separated, washed with water, brine, dried over Na₂SO₄, filtered, and concentrated in vacuo to afford Intermediate 595 (726 mg, yield: 79%).

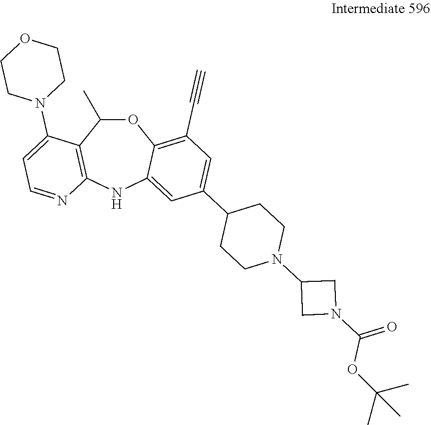 [SMILES: C#Cc1cc(C2CCN(C3CN(C(=O)OC(C)(C)C)C3)CC2)cc2c1OC(C)c1c(N3CCOCC3)ccnc1N2]

TBAF (1 M in THF, 1.86 mL, 1.86 mmol) was added to a stirred solution of Intermediate 595 (720 mg, 0.93 mmol) in THF (9.4 mL) at room temperature and the mixture was stirred for 1.5 h. The mixture was diluted with EtOAc and washed with water. The organic layer was separated, dried over Na₂SO₄, filtered, and concentrated in vacuo. Purification by flash column chromatography (silica, EtOAc in heptane 60/40 to 100/0) afforded Intermediate 596 (394 mg, yield: 70%).

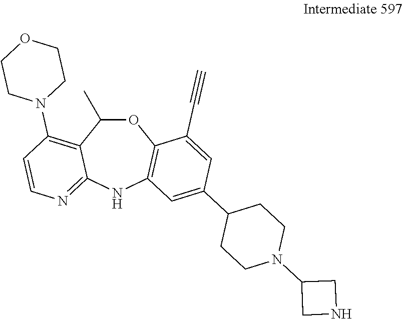 [SMILES: C#Cc1cc(C2CCN(C3CNC3)CC2)cc2c1OC(C)c1c(N3CCOCC3)ccnc1N2]

HCl (4 M in 1,4-dioxane, 0.51 mL, 2.04 mmol) was added to a stirred solution of Intermediate 596 (394 mg, 0.68 mmol) in 1,4-dioxane (5.58 mL) at room temperature and the mixture was stirred for 16 h. Then, more HCl (4 M in 1,4-dioxane, 0.17 mL, 0.68 mmol) was added and the mixture was stirred at room temperature for 4 h. The mixture was then treated with a solution of NH₃ in MeOH (7 M) and concentrated in vacuo. The residue was purified by flash column chromatography (silica-NH₂, MeOH in DCM 0/100 to 5/95) to afford Intermediate 597 (232 mg, yield: 70%).

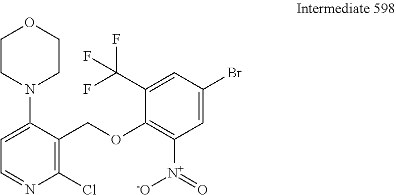

At 0° C., DIAD (CAS [2446-83-5], 1.672 mL, 8.492 mmol) was added dropwise to a mixture of 4-bromo-2-nitro-6-(trifluoromethyl)phenol [CAS: 2089255-50-3] (1.76 g, 6.154 mmol), Intermediate 59 (1.584 g, 6.509 mmol), and PPh₃ (2.253 g, 8.589 mmol) in THF (81 mL). The reaction mixture was stirred at room temperature overnight, then poured into water, and extracted with EtOAc. The organic layer was separated, dried, filtered, and the solvent was evaporated until dryness. Purification by column chromatography (Irregular SiOH 15-45 μm 40 g GraceResolv®, mobile phase Gradient from 100% heptane to 60% heptane, 40% EtOAc) gave Intermediate 598 (2.95 g, yield: 96%).

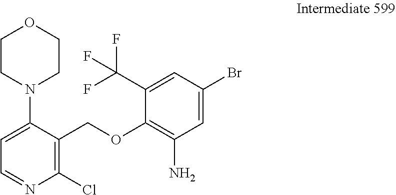 [SMILES: Nc1cc(Br)cc(C(F)(F)F)c1OCc1c(N2CCOCC2)ccnc1Cl]

Intermediate 598 (2.96 g, 5.96 mmol), iron powder (3.35 g, 59.985 mmol) and MeOH (35 mL) were stirred with glacial AcOH (6.8 mL) at room temperature for 5 h. This reaction mixture was poured into aqueous NaHCO₃ and DCM, and the organic layer was decanted over Chromabond®. The solvent was evaporated until dryness. Purification by column chromatography (Irregular SiOH 15-40 μm 40 g GraceResolv®, Gradient from 100% DCM to 94% DCM, 6% MeOH, 0.6% NH₄OH) afforded Intermediate 599 (2.26 g, yield: 81%).

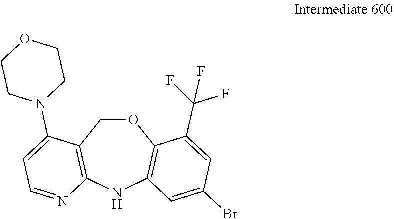 [SMILES: FC(F)(F)c1cc(Br)cc2c1OCc1c(N3CCOCC3)ccnc1N2]

A mixture of Intermediate 599 (2.26 g, 4.843 mmol) in t-amyl alcohol (18 mL) was stirred at 140° C. for 2 h. The reaction mixture was cooled down to room temperature and evaporated until dryness. This crude mixture was taken up with diethyl ether and ACN (90/10) and was triturated. The precipitate was filtered and dried to afford a first batch of Intermediate 600. The filtrate was evaporated until dryness and was purified by column chromatography (Irregular SiOH 15-40 μm 25 g GraceResolv®, Gradient from heptane/EtOAc 80/20 to 40/60) to afford a second batch of Intermediate 600 (combined batches: 1.25 g, combined yield: 60%).

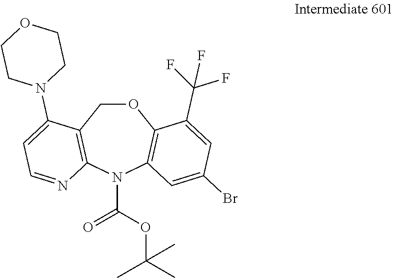 [SMILES: CC(C)(C)OC(=O)N1c2cc(Br)cc(C(F)(F)F)c2OCc2c(N3CCOCC3)ccnc21]

A mixture of Intermediate 600 (570 mg, 1.325 mmol), di-tert-butyl dicarbonate [CAS: 24424-99-5] (578 mg, 2.648 mmol), DMAP [CAS: 1122-58-3] (33 mg, 0.27 mmol), and Et₃N (0.37 mL, 2.662 mmol) in DCM (10 mL) was stirred at room temperature for 2 days. The reaction mixture was poured into water and extracted with EtOAc. The combined organic layer was dried, concentrated to dryness, and purified by column chromatography (Irregular SiOH 15-40 μm 80 g GraceResolv®, gradient from 99% DCM, 1% MeOH, 0.1% NH₄OH to 95% DCM, 5% MeOH, 0.5% NH₄OH) to afford Intermediate 601 (452 mg, yield: 64%).

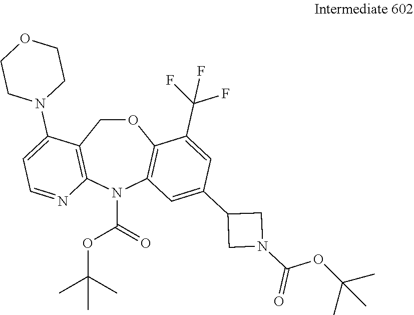 [SMILES: CC(C)(C)OC(=O)N1CC(c2cc3c(c(C(F)(F)F)c2)OCc2c(N4CCOCC4)ccnc2N3C(=O)OC(C)(C)C)C1]

A mixture of Intermediate 601 (450 mg, 0.849 mmol), 1,1′-Pd(dppf)₂Cl₂ [CAS: 95464-05-4] (35 mg, 0.0424 mmol), and CuI [CAS: 7681-65-4] (16 mg, 0.084 mmol) in DMA (4.8 mL) was purged under nitrogen flux in a sealed tube 3 times. [1-[(1,1-Dimethylethoxy)carbonyl]-3-azetidinyl]iodozine [CAS: 206446-38-0] (7 mL, 0.24 M, 1.68 mmol) was added and the mixture was degassed 3 times. The reaction was stirred at 80° C. under microwave irradiation for 30 min. The reaction mixture was cooled down to room temperature, poured into water and EtOAc, and stirred for 10 min at room temperature. The organic layer was separated, dried (MgSO₄), filtered, and the solvent was evaporated until dryness. Purification by column chromatography (Irregular SiOH 15-40 μm 40 g GraceResolv®, gradient from 98% DCM, 2% MeOH, 0.2% NH₄OH to 94% DCM, 6% MeOH, 0.6% NH₄OH) afforded Intermediate 602 (250 mg, yield: 49%).

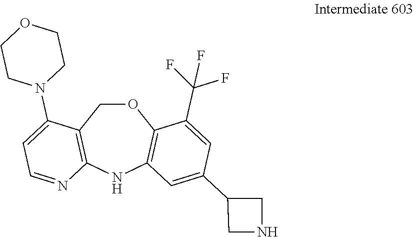 [SMILES: FC(F)(F)c1cc(C2CNC2)cc2c1OCc1c(N3CCOCC3)ccnc1N2]

HCl (4 M in 1,4-dioxane, 1.6 mL, 6.4 mmol) was added slowly to a solution of Intermediate 602 (250 mg, 0.412 mmol) in 1,4-dioxane (5 mL) at 0° C. The reaction mixture was stirred at room temperature for 5 h, then overnight before the solvent was evaporated until dryness to give Intermediate 603 (251 mg, quant yield).

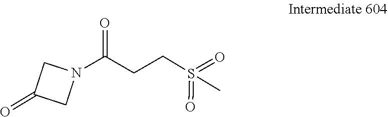

HBTU [CAS: 94790-37-1] (1.5 g, 3.94 mmol) was added to a solution of 3-(methylsulfonyl)propanoic acid [CAS: 645-83-0] (0.5 g, 3.29 mmol), 3-azetidone, HCl salt [CAS: 17557-84-5] (0.35 g, 3.29 mmol), and Et₃N (0.69 mL, 4.93 mmol) in DCM (10 mL), and the mixture was stirred at room temperature for 3 days. The solvent was removed and purification on silica chromatography (eluent DCM: DCM:MeOH (9:1)) afforded Intermediate 604 (674 mg, impure, assumed quantitative).

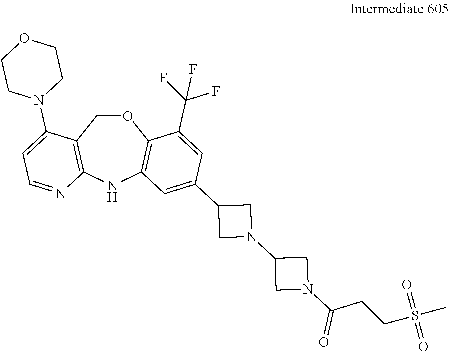 [SMILES: CS(=O)(=O)CCC(=O)N1CC(N2CC(c3cc4c(c(C(F)(F)F)c3)OCc3c(N5CCOCC5)ccnc3N4)C2)C1]

A mixture of Intermediate 603 (197 mg, 0.411 mmol), Intermediate 604 (130 mg, 0.633 mmol, 1.5 eq.), AcOH (0.05 mL, 0.873 mmol, 2.1 eq.), and sodium triacetoxyborohydride (170 mg, 0.802 mmol, 1.9 eq.) in DCM (4 mL) was stirred at room temperature for 2 days. Water and K₂CO₃ (10% in water) were added and the aqueous phase was extracted with EtOAc and with DCM. The combined organic layer was evaporated. The residue was purified by column chromatography (Irregular SiOH 15-40 μm 40 g GraceResolv®; from 97% DCM, 3% MeOH, 0.3% NH₄OH to 85% DCM, 15% MeOH, 1.5% NH₄OH) to afford Intermediate 605 (28 mg, yield: 11%).

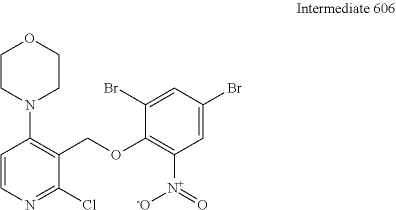

Intermediate 606 was synthesized in a similar manner as Intermediate 539 using 2,4-dibromo-6-nitrophenol [CAS: 15969-09-2] instead of Intermediate 538.

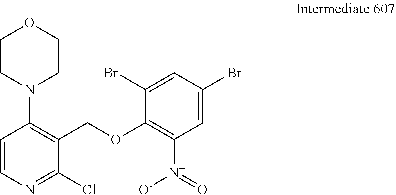 [SMILES: O=[N+]([O-])c1cc(Br)cc(Br)c1OCc1c(N2CCOCC2)ccnc1Cl]

Intermediate 607 was synthesized in a similar manner as Intermediate 584 using Intermediate 606 instead of Intermediate 583.

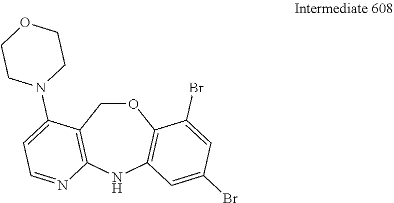 [SMILES: Brc1cc(Br)c2c(c1)Nc1nccc(N3CCOCC3)c1CO2]

TFA 1.55 mL was added to a stirred solution of Intermediate 607 (3.22 g, 6.74 mmol) in 1,4-dioxane (40 mL) and the mixture was stirred at 120° C. for 20 h. The reaction mixture was allowed to cool to room temperature and was diluted with DCM. The mixture was washed with aqueous Na₂CO₃ (1 M). The organic layer was separated, dried over MgSO₄, filtered, and concentrated to dryness. Chromatography over silica gel (gradient of EtOAc in heptane from 0 to 50%) followed by trituration in ACN and repurification of the filtrate by reverse phase chromatography (Phenomenex Gemini C18 100×30 mm 5 μm; gradient of ACN/MeOH (1/1, v/v) in 25 mM aqueous ammonium bicarbonate from 51 to 94%) gave Intermediate 608 (2.21 g, yield: 72%).

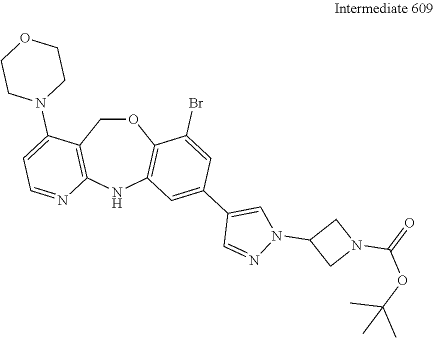 [SMILES: CC(C)(C)OC(=O)N1CC(n2cc(-c3cc(Br)c4c(c3)Nc3nccc(N5CCOCC5)c3CO4)cn2)C1]

Intermediate 608 (1.1 g, 2.49 mmol), 1-azetidinecarboxylic acid, 3-[4-(4,4,5,5-tetramethyl-1,3,2-dioxaborolan-2-yl)-1H-pyrazol-1-yl]-, 1,1-dimethylethyl ester [CAS: 877399-35-4] (2.18 g, 3.74 mmol), and Pd(dppf)Cl₂·DCM [CAS: 95464-05-4] (0.12 g, 0.15 mmol) were placed in a mixture of aqueous Na₂CO₃ (1 M, 3.74 mL) and 1,4-dioxane (20 mL) and bubbled with nitrogen for 15 min. The mixture was then maintained under nitrogen atmosphere and heated at 80° C. for 4 h. The reaction mixture was allowed to cool to room temperature and was diluted with EtOAc. Water and brine were added. The organic layer was separated and the aqueous layer was extracted with EtOAc. The combined organic layers were dried over MgSO₄, filtered, and concentrated. Chromatography over silica gel (gradient of EtOAc in heptane from 25 to 100%) afforded Intermediate 609 (368 mg, 85% purity, yield: 22%).

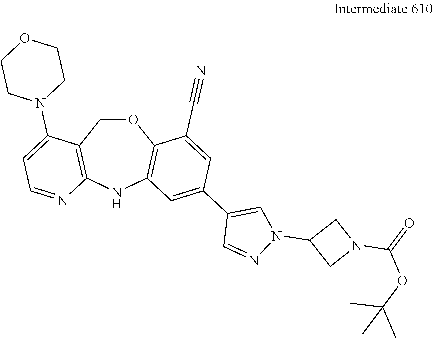 [SMILES: CC(C)(C)OC(=O)N1CC(n2cc(-c3cc(C#N)c4c(c3)Nc3nccc(N5CCOCC5)c3CO4)cn2)C1]

CuCN [CAS: 544-92-3] (138 mg, 1.54 mmol) was added to a solution of Intermediate 609 (359 mg, 0.62 mmol) in DMF (15 mL) under nitrogen atmosphere. The solution was heated at 140° C. for 20 h, then the mixture was allowed to cool to room temperature and was diluted with EtOAc. Aqueous Na₂CO₃ (1 M) was added and the solids were filtered off through a short pad of Celite. The organic layer was separated, washed with brine, dried over MgSO₄, filtered, and concentrated to dryness. Chromatography over silica gel (gradient of MeOH in DCM from 0 to 3%) afforded Intermediate 610 (101 mg, 90% purity, yield: 28%).

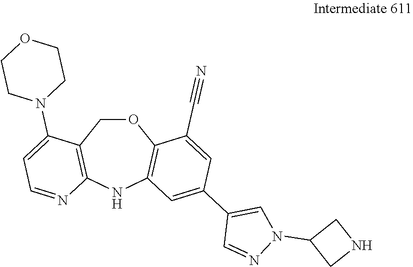 [SMILES: N#Cc1cc(-c2cnn(C3CNC3)c2)cc2c1OCc1c(N3CCOCC3)ccnc1N2]

Intermediate 611 was synthesized in a similar manner as Intermediate 588 using Intermediate 610 instead of Intermediate 587A.

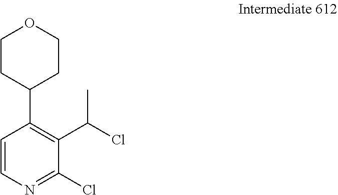 [SMILES: CC(Cl)c1c(C2CCOCC2)ccnc1Cl]

Thionyl chloride (0.6 mL, 7.98 mmol) was added dropwise to a mixture of Intermediate 480 (0.96 g, 3.99 mmol) in DCM (20 mL) at 0° C. under nitrogen atmosphere. The reaction mixture was stirred at room temperature for 3 h, then concentrated to dryness. Water and DCM were added and the layers were separated. The organic layer was dried over MgSO₄, filtered, and concentrated to dryness to give Intermediate 612 (1.37 g, 76% purity, quantitative yield).

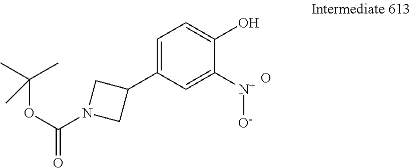

Sodium nitrite (3.44 g, 49.82 mmol) and potassium hydrogenosulfate [CAS: 7646-93-7] (11.8 g, 86.64 mmol) were added to a solution of 1-azetidinecarboxylic acid, 3-(4-hydroxyphenyl)-, 1,1-dimethylethyl ester [CAS: 1782327-13-2], 5.4 g, 21.66 mmol) in ACN (130 mL) and the mixture was stirred at 50° C. overnight. The mixture was cooled to room temperature and diluted with EtOAc, then washed with brine, dried (MgSO₄), and concentrated. Purification by column flash chromatography (SiO₂, EtOAc-heptane gradient) afforded Intermediate 613 (3.23 g, yield: 51%).

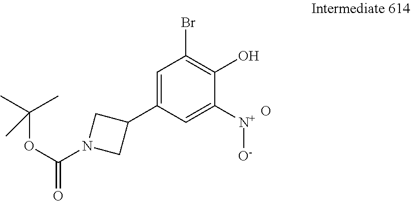

A solution of bromine (1.7 g, 10.62 mmol) in AcOH (4 mL) was added dropwise to a solution of Intermediate 613 (2.61 g, 8.85 mmol) in AcOH (32 mL) and MeOH (36 mL) and the reaction was stirred at room temperature for 2 h. The reaction mixture was diluted with DCM and water. The layers were separated and the organic layer was dried over MgSO₄, filtered, and evaporated in vacuo. Purification by flash column chromatography (silica gel, EtOAc/heptane, from 0/100 to 30/70) afforded Intermediate 614 (1.7 g, yield: 46%).

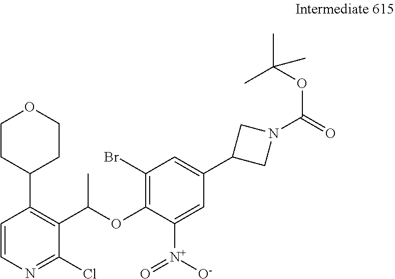 [SMILES: CC(Oc1c(Br)cc(C2CN(C(=O)OC(C)(C)C)C2)cc1[N+](=O)[O-])c1c(C2CCOCC2)ccnc1Cl]

Intermediate 612 (2.96 g, 11.36 mmol) was added to a mixture of Intermediate 614 (2.12 g, 5.68 mmol) and K₂CO₃ (1.57 g, 11.36 mmol) in DMF (60 mL). The reaction mixture was stirred at 80° C. for 2 days. Water and EtOAc were added and the organics were separated. The organic layer was dried over MgSO₄, filtered, and concentrated in vacuo. The residue was purified by flash column chromatography (silica gel, EtOAc/heptane, from 0/100 to 40/60) to afford Intermediate 615 (2.59 g, yield: 68%).

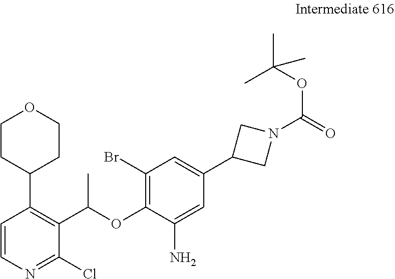 [SMILES: CC(Oc1c(N)cc(C2CN(C(=O)OC(C)(C)C)C2)cc1Br)c1c(C2CCOCC2)ccnc1Cl]

Intermediate 615 (2.64 g, 4.42 mmol) was suspended in a mixture of AcOH (5 mL) and MeOH (45 mL), then iron powder (2.47 g, 44.14 mmol) was added and the mixture was stirred at room temperature overnight. The excess iron was removed and the mixture was diluted with DCM and quenched with saturated aqueous Na₂CO₃. The organic layer was dried over MgSO₄, filtered, and the solvent was evaporated in vacuo. The residue was purified by flash column chromatography (silica gel, EtOAc/heptane from 0/100 to 65/35) to afford Intermediate 616 (2.08 g, yield: 78%).

### Intermediate 617

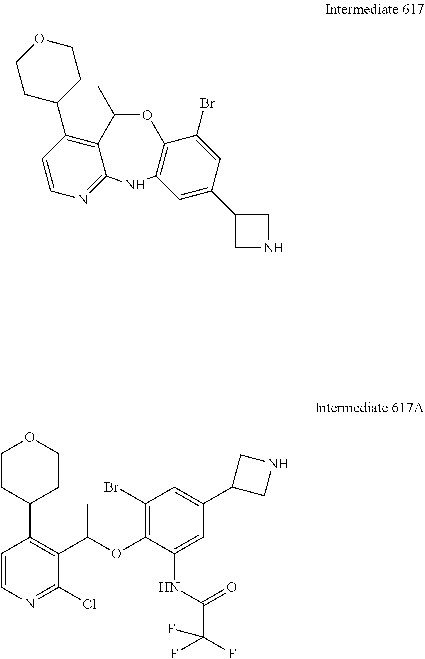 [SMILES: CC1Oc2c(Br)cc(C3CNC3)cc2Nc2nccc(C3CCOCC3)c21 ; CC(Oc1c(Br)cc(C2CNC2)cc1NC(=O)C(F)(F)F)c1c(C2CCOCC2)ccnc1Cl]

Intermediate 616 (2.07 g, 3.65 mmol) was dissolved in 1,4-dioxane (20 mL), then TFA (0.84 mL, 10.96 mmol) was added, and the reaction mixture was stirred at 120° C. overnight. Water (0.5 mL) was added and the mixture was stirred at 120° C. for 2 h. After cooling, the reaction mixture was concentrated to dryness. The residue was diluted with DCM and washed with 1 M aqueous Na₂CO₃. The organic layer was dried over MgSO₄, filtered, and evaporated in vacuo. Purification by flash column chromatography (silica gel, DCM+MeOH+NH₄OH (9/0.9/0.1)/DCM, from 0/100 to 100/0) afforded Intermediate 617 (283 mg, yield: 14%) and Intermediate 617A (745 mg, 69% purity, yield: 25%). The fraction containing Intermediate 617A was dissolved in 1,4-dioxane (6 mL) and Cs₂CO₃ (0.74 g, 2.27 mmol) was added. The reaction mixture was stirred at reflux overnight. After cooling, the mixture was diluted with DCM and washed with water. The organic layer was dried over MgSO₄, filtered, and evaporated. The residue was purified by flash column chromatography over silica gel, (DCM+MeOH+NH4OH (9/0.9/0.1)/DCM, from 0/100 to 100/0) to afford another batch of Intermediate 617 (110 mg, yield: 20%).

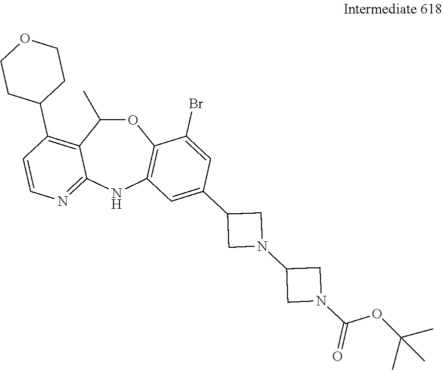 [SMILES: CC1Oc2c(Br)cc(C3CN(C4CN(C(=O)OC(C)(C)C)C4)C3)cc2Nc2nccc(C3CCOCC3)c21]

Intermediate 617 (380 mg, 0.89 mmol) was dissolved in DCE (4 mL). 1-Boc-3-azetidinone [CAS: 398489-26-4] (0.3 g, 1.78 mmol) and AcOH (0.05 mL) were added and, after 30 min, sodium triacetoxyborohydride (0.28 g, 1.33 mmol) was added portionwise. The reaction mixture was stirred at room temperature overnight. The mixture was diluted with EtOAc and washed with aqueous Na₂CO₃ (1 M). The aqueous layers were extracted once more time with EtOAc and the combined organic layers were dried over MgSO₄, filtered, and concentrated to dryness. The residue was purified by flash column chromatography (silica gel, EtOAc/heptane, from 0/100 to 100/0) to afford Intermediate 618 (340 mg, 56%).

### Intermediate 619A and Intermediate 619B

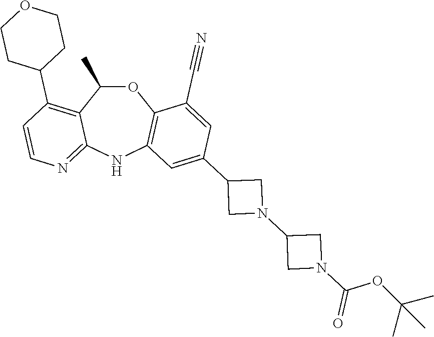 [SMILES: C[C@H]1Oc2c(C#N)cc(C3CN(C4CN(C(=O)OC(C)(C)C)C4)C3)cc2Nc2nccc(C3CCOCC3)c21]

Intermediate 619A: (*R) pure stereoisomer but absolute stereochemistry undetermined.

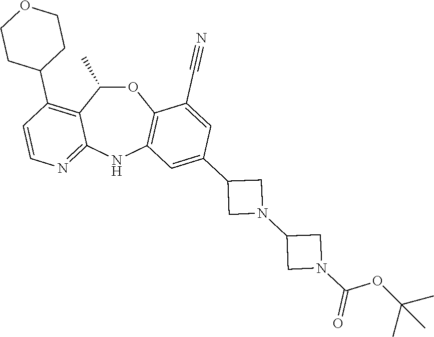 [SMILES: C[C@@H]1Oc2c(C#N)cc(C3CN(C4CN(C(=O)OC(C)(C)C)C4)C3)cc2Nc2nccc(C3CCOCC3)c21]

Intermediate 619B: (*S) pure stereoisomer but absolute stereochemistry undetermined.

Intermediate 618 (320 mg, 0.54 mmol) was dissolved in DMA (6 mL) and degassed by bubbling with nitrogen. Zinc cyanide (38 mg, 0.32 mmol), Pd(dppf)Cl₂·DCM (24 mg, 0.027 mmol), and zinc powder (1 mg, 0.011 mmol) were added and bubbling was continued for 15 min. The mixture was then placed under nitrogen atmosphere and heated at 120° C. for 3 h in a sealed tube. More Pd(dppf)C12.DCM (24 mg, 0.027 mmol) and zinc powder (1 mg, 0.011 mmol) were added and bubbling was continued for 15 min. The mixture was then placed under nitrogen atmosphere and heated at 120° C. overnight in a sealed tube. The mixture was allowed to cool to room temperature, diluted with water and EtOAc. The organic layer was separated, washed with brine, dried over MgSO₄, filtered, and concentrated to dryness. Chromatography over silica gel (gradient of EtOAc in heptane from 0/100 to 100/0) followed by reverse phase chiral chromatography (Phenomenex Lux Cellulose-1 150×21.2 mm 5 μm; gradient from 75% [heptane+0.1% DEA]−25% [iPrOH+0.1% DEA] to 100% [iPrOH+0.1% DEA] to afford Intermediate 619A (67 mg, yield: 22%) and Intermediate 619B (74 mg, yield: 25%).

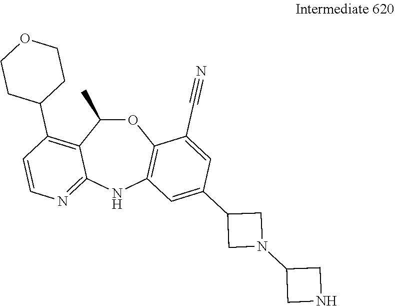 [SMILES: C[C@H]1Oc2c(C#N)cc(C3CN(C4CNC4)C3)cc2Nc2nccc(C3CCOCC3)c21]

(*R) pure stereoisomer but absolute stereochemistry undetermined.

Intermediate 619A (67 mg, 0.13 mmol) was dissolved in DCM (4 mL). The mixture was cooled to 0° C. and TFA (0.27 mL) was added. The mixture was stirred at room temperature overnight. The solvent was evaporated and the residual TFA was co-evaporated with toluene. The residue was taken up in DCM and treated with Amberlyst A26 hydroxide [CAS: 39339-85-0] until pH 7. The resin was filtered off and the process was repeated 5 times. The filtrate was concentrated to dryness and dried under high vacuum to afford Intermediate 620 (47 mg, yield: 86%).

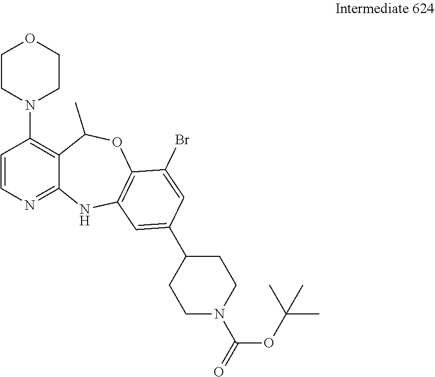 [SMILES: CC1Oc2c(Br)cc(C3CCN(C(=O)OC(C)(C)C)CC3)cc2Nc2nccc(N3CCOCC3)c21]

Intermediate 621 was synthesized in a similar manner as Intermediate 193 using Intermediate 492 instead of Intermediate 192.

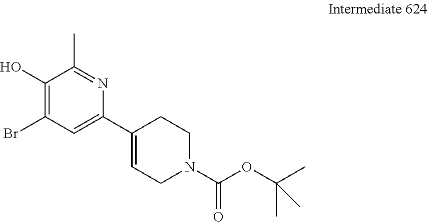 [SMILES: Cc1nc(C2=CCN(C(=O)OC(C)(C)C)CC2)cc(Br)c1O]

Pd(dppf)Cl₂ DCM [CAS: 95464-05-4] (1.86 g, 2.27 mmol) was added to a suspension of 4,6-dibromo-2-methyl-3-pyridinol [CAS: 188923-75-3] (12.1 g, 45.33 mmol), N-Boc-1,2,3,6-tetrahydropyridine-4-boronic acid pinacol ester [CAS: 286961-14-6](11.21 g, 36.27 mmol), and K₃PO₄ (19.25 g, 90.66 mmol) in a mixture of 1,4-dioxane (192 mL) and water (32 mL), under nitrogen atmosphere. The mixture was stirred overnight at room temperature. The reaction mixture was partitioned between EtOAc and brine. The combined organic layers were separated, dried over MgSO₄, filtered, and concentrated to dryness. The residue was purified by column chromatography on silica gel (heptane and EtOAc) to afford Intermediate 624 (5.69 g, yield: 34%).

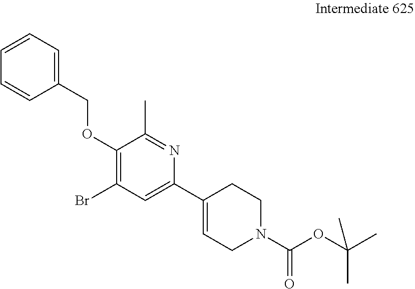 [SMILES: Cc1nc(C2=CCN(C(=O)OC(C)(C)C)CC2)cc(Br)c1OCc1ccccc1]

Benzyl bromide [CAS: 100-39-0] (0.73 mL, 6.1 mmol) was added to a solution of Intermediate 624 (1.69 g, 4.07 mmol) and K₂CO₃ (0.67 g, 4.88 mmol) in acetone (25 mL). The reaction mixture was stirred at 50° C. for 3 h, then filtered, and water and brine were added. The mixture was extracted with EtOAc and the organic layer was separated, dried over MgSO₄, filtered, concentrated to dryness and purified by column chromatography over silica gel (gradient of EtOAc in heptane from 0% to 50%) to afford Intermediate 625 (1.86 g, 86% purity, yield: 85%).

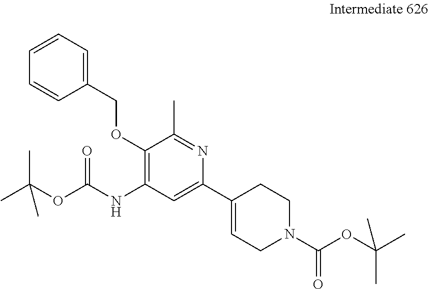 [SMILES: Cc1nc(C2=CCN(C(=O)OC(C)(C)C)CC2)cc(NC(=O)OC(C)(C)C)c1OCc1ccccc1]

Intermediate 626 was synthesized in a similar manner as Intermediate 71 using Intermediate 625 instead of Intermediate 70 using 2 eq of Cs₂CO₃ and stirring the reaction at 100° C.

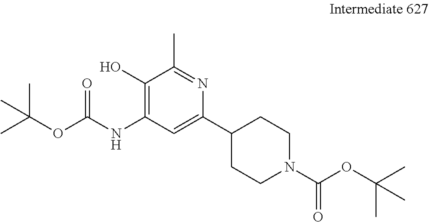 [SMILES: Cc1nc(C2CCN(C(=O)OC(C)(C)C)CC2)cc(NC(=O)OC(C)(C)C)c1O]

Pd/C (10%, 430 mg) was added to an ice-cold solution of Intermediate 626 (3.28 g, 6.62 mmol) in MeOH (30 mL). The reaction mixture was hydrogenated at room temperature under H₂ atmosphere overnight. The mixture was filtered over celite and evaporated until dryness to yield Intermediate 627 (2.66 g, yield: 94%).

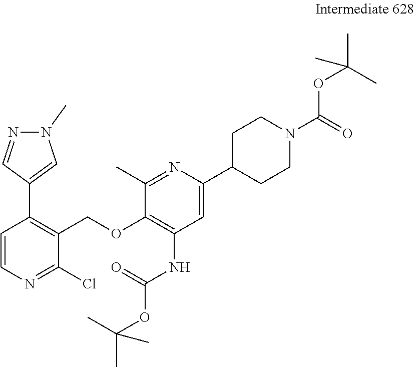 [SMILES: Cc1nc(C2CCN(C(=O)OC(C)(C)C)CC2)cc(NC(=O)OC(C)(C)C)c1OCc1c(-c2cnn(C)c2)ccnc1Cl]

Intermediate 627 (2.66 g, 6.53 mmol), Intermediate 45 (2.37 g, 9.79 mmol), and K₂CO₃ (1.8 g, 13.06 mmol) were stirred at room temperature in DMF (70 mL) for 20 h. The reaction mixture was diluted with water and EtOAc. The organic layer was separated and the aqueous phase was extracted with EtOAc. The combined organic layers were washed with brine, dried over MgSO₄, filtered, concentrated, and purified by chromatography over silica gel (gradient of EtOAc in heptane from 25 to 100%, then holding 100% EtOAc) to afford Intermediate 628 (4.1 g, 90% purity, yield: 92%).

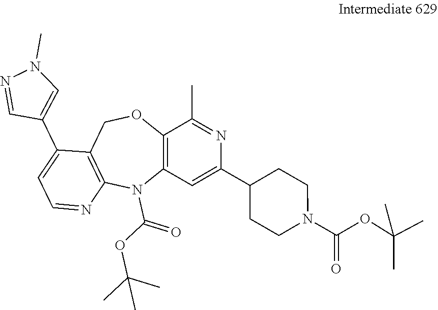 [SMILES: Cc1nc(C2CCN(C(=O)OC(C)(C)C)CC2)cc2c1OCc1c(-c3cnn(C)c3)ccnc1N2C(=O)OC(C)(C)C]

Intermediate 628 (18 g, 29.36 mmol) and Cs₂CO₃ (14.35 g, 44.04 mmol) were suspended in toluene (250 mL) and bubbled with nitrogen for 15 min. Palladium(II) acetate [CAS: 3375-31-3] (0.34 g, 2.94 mmol) and Xantphos [CAS: 161265-03-8] (1.7 g, 2.94 mmol) were then added and resulting mixture was heated to 120° C. under nitrogen atmosphere. The reaction was continued for 3 h and then allowed to cool to room temperature. The reaction mixture was filtered and the filtrate was diluted with water and EtOAc. The mixture was filtered through a pad of Celite, and the aqueous layer was extracted with EtOAc. The combined organic layers were dried over MgSO₄, filtered, concentrated, and purified with chromatography over silica gel (gradient of EtOAc in heptane from 25 to 100%) to afford Intermediate 629 (13.8 g, yield: 79%).

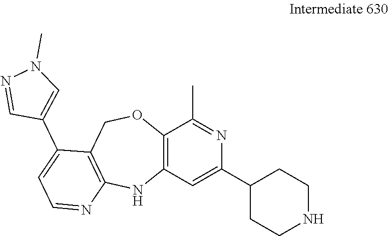 [SMILES: Cc1nc(C2CCNCC2)cc2c1OCc1c(-c3cnn(C)c3)ccnc1N2]

Intermediate 629 (13.8 g, 23.93 mmol) was dissolved in DCM (150 mL) and treated with TFA (48.5 mL, 634 mmol) at 0° C. The mixture was then allowed to warm up to room temperature and was stirred for 5 h. The reaction mixture was concentrated under reduced pressure. The residue was suspended in toluene and again concentrated to dryness and dried under high vacuum at 60° C. to constant weight to afford Intermediate 630 (26.6 g, quantitative yield).

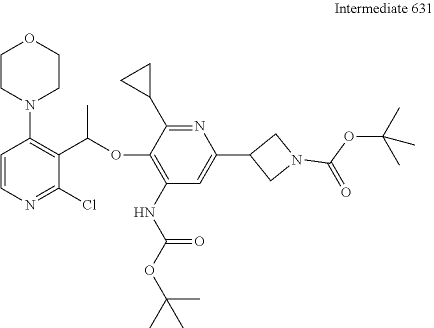 [SMILES: CC(Oc1c(NC(=O)OC(C)(C)C)cc(C2CN(C(=O)OC(C)(C)C)C2)nc1C1CC1)c1c(N2CCOCC2)ccnc1Cl]

K₂CO₃ (6.63 g, 47.969 mmol, 4 eq.) was added to a suspension of Intermediate 640 (5.3 g, 11.992 mmol) in DMF (40 mL). The reaction mixture was stirred at room temperature, then Intermediate 27 (3.132 g, 11.992 mmol, 1 eq.) was added in small portions over 5 h. The mixture was stirred at room temperature for 16 h. To push the reaction to completion, more Intermediate 27 (2.349 g, 8.994 mmol, 0.75 eq.) was added and the reaction mixture was stirred at room temperature for 18 h. The mixture was diluted with EtOAc and washed with brine (5 times). The organic layer was concentrated and the residue was purified by flash chromatography (SiO₂, MeOH-DCM) to give Intermediate 631 (5.3 g, yield: 70%).

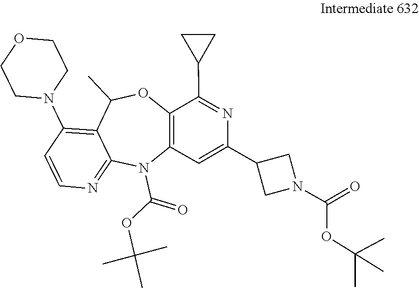 [SMILES: CC1Oc2c(cc(C3CN(C(=O)OC(C)(C)C)C3)nc2C2CC2)N(C(=O)OC(C)(C)C)c2nccc(N3CCOCC3)c21]

A solution of Intermediate 631 (5.3 g, 8.41 mmol) and Cs₂CO₃ (4.11 g, 12.616 mmol, 1.5 eq.) in toluene (150 mL) was degassed under nitrogen atmosphere. Xantphos (CAS [161265-03-8], 730 mg, 1.262 mmol, 0.15 eq.) and Pd(OAc)₂ (283 mg, 1.262 mmol, 0.15 eq.) were added. The reaction mixture was degassed again under nitrogen and stirred at 100° C. for 15 h. To push the reaction to completion, more Xantphos (CAS [161265-03-8], 730 mg, 1.262 mmol, 0.15 eq.) and Pd(OAc)₂ (283 mg, 1.262 mmol, 0.15 eq.) were added and the mixture was stirred under nitrogen atmosphere at 100° C. for 72 h. The reaction mixture was partitioned between EtOAc and brine. The combined organic layers were concentrated and the crude was purified by flash chromatography (EtOAc-heptane) to give Intermediate 632 (3.96 g, yield: 79%).

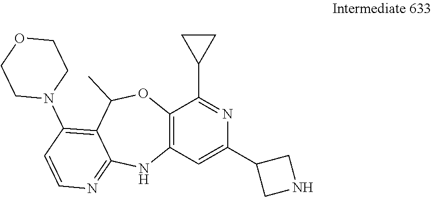 [SMILES: CC1Oc2c(cc(C3CNC3)nc2C2CC2)Nc2nccc(N3CCOCC3)c21]

Intermediate 632 (3.96 g, 6.67 mmol) was added to a mixture of TFA (14 mL) and DCM (21 mL) and the reaction mixture was stirred for 3 h at room temperature. The volatiles were evaporated and the residue was co-evaporated with toluene (2×100 mL) and dried to give Intermediate 633 (TFA salt, 7.72 g, quantitative yield), used as such without further purification.

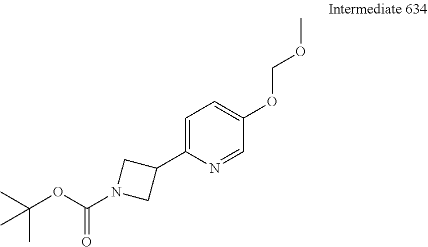 [SMILES: COCOc1ccc(C2CN(C(=O)OC(C)(C)C)C2)nc1]

Intermediate 634 was synthesized in a similar manner as Intermediate 526 using 2-chloro-5-(methoxymethoxy)pyridine [CAS: 877133-56-7] instead of Intermediate 525 and using 0.03 eq of Pd(dppf)Cl₂. DCM [CAS: 95464-05-4], 0.06 eq of CuI [CAS: 7681-65-4], and 1.25 eq of [1-[(1,1-dimethylethoxy)carbonyl]-3-azetidinyl]iodozine [CAS: 206446-38-0].

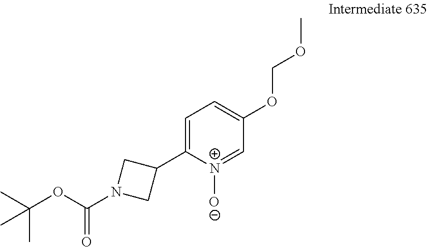 [SMILES: COCOc1ccc(C2CN(C(=O)OC(C)(C)C)C2)[n+]([O-])c1]

Intermediate 634 (43.56 g, 148 mmol) was dissolved in DCM (400 mL), then a dry solution of 3-chloroperbenzoic acid [CAS: 937-14-4] (49.75 g, 222 mmol) in DCM (200 mL) was added at room temperature dropwise. The reaction mixture was stirred for 15 h. The mixture was poured into saturated aqueous NaHCO₃ and extracted with DCM, then the organics were combined, dried over MgSO₄, filtered, and concentrated to dryness. Purification by column flash chromatography (silica; DCM-MeOH gradient) afforded Intermediate 635 (44.16 g, yield: 96%).

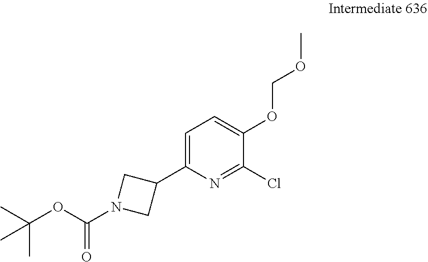 [SMILES: COCOc1ccc(C2CN(C(=O)OC(C)(C)C)C2)nc1Cl]

Intermediate 635 (22.8 g, 73.47 mmol) and Et₃N (102 mL, 734.66 mmol) were dissolved in DCE (170 mL), then phosphorus(V) oxychloride [CAS: 10025-87-3] (6.85 mL, 73.47 mmol) was added under a nitrogen atmosphere and the resulting mixture was brought to reflux for 30 min. An aqueous solution of NaHCO₃ and ice was added and the organic layer was extracted with DCM, dried over MgSO₄, filtered, and concentrated to dryness. Purification by column chromatography over silica gel (gradient of EtOAc in heptane) afforded Intermediate 636 (6.9 g, yield: 29%).

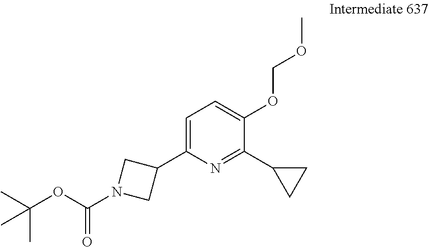 [SMILES: COCOc1ccc(C2CN(C(=O)OC(C)(C)C)C2)nc1C1CC1]

A solution of Intermediate 636 (3.58 g, 10.89 mmol), cyclopropyl boronic acid [CAS: 411235-57-9] (2.81 g, 32.67 mmol), and potassium phosphate [CAS: 7778-53-2] (6.93 g, 32.67 mmol) in 1,4-dioxane (76 mL) and water (11 mL) was degassed under a nitrogen atmosphere. Then Pd(dppf)Cl₂. DCM [CAS: 95464-05-4] (0.89 g, 1.09 mmol) was added. The reaction mixture was degassed again under a nitrogen atmosphere and heated at 100° C. for 16 h. The reaction mixture was partitioned between EtOAc and brine, the combined organic layers were dried over MgSO₄, filtered, and concentrated. Purification by column flash chromatography (silica; heptane/EtOAc) afforded Intermediate 637 (2.28 g, yield: 62%).

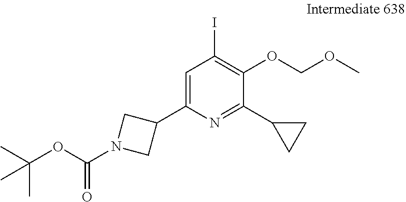 [SMILES: COCOc1c(I)cc(C2CN(C(=O)OC(C)(C)C)C2)nc1C1CC1]

Intermediate 638 was synthesized in a similar manner as Intermediate 82 using Intermediate 637 instead of Intermediate 81.

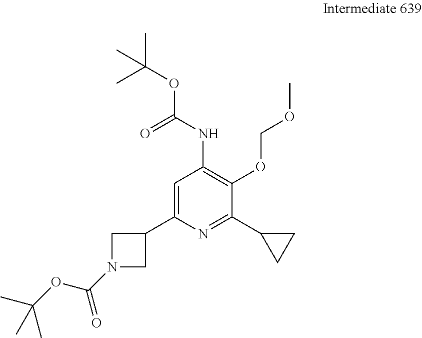 [SMILES: COCOc1c(NC(=O)OC(C)(C)C)cc(C2CN(C(=O)OC(C)(C)C)C2)nc1C1CC1]

Intermediate 639 was synthesized in a similar manner as Intermediate 83 using Intermediate 638 instead of Intermediate 82.

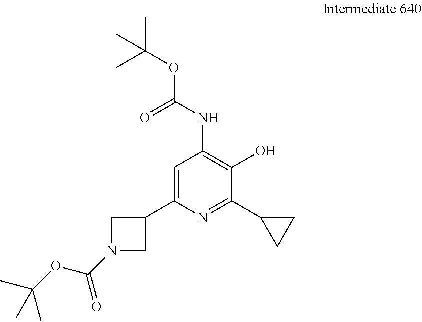 [SMILES: CC(C)(C)OC(=O)Nc1cc(C2CN(C(=O)OC(C)(C)C)C2)nc(C2CC2)c1O]

Intermediate 640 was synthesized in a similar manner as Intermediate 84 using Intermediate 639 instead of Intermediate 83, using Intermediate 640 without further purification after the work-up.

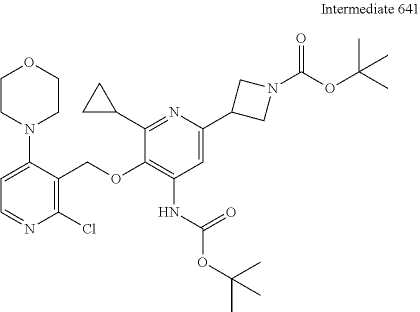 [SMILES: CC(C)(C)OC(=O)Nc1cc(C2CN(C(=O)OC(C)(C)C)C2)nc(C2CC2)c1OCc1c(N2CCOCC2)ccnc1Cl]

K₂CO₃ (2.56 g, 18.5 mmol, 3 eq.) was added to a solution of Intermediate 640 (2.5 g, 6.17 mmol) and Intermediate 60 (2.29 g, 9.25 mmol) in DMF (37 mL). The reaction mixture was stirred at room temperature for 15 h and then diluted with EtOAc. The organic layer was washed with brine, dried over MgSO₄, filtered, concentrated, and purified by column chromatography over silica gel (gradient of EtOAc in heptane) to afford Intermediate 641 (1.51 g, yield: 40%).

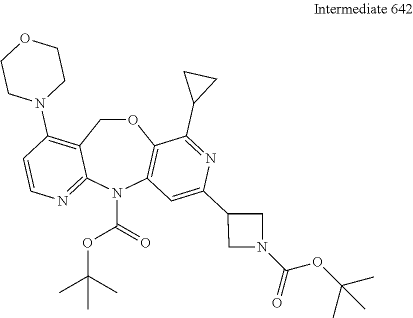 [SMILES: CC(C)(C)OC(=O)N1CC(c2cc3c(c(C4CC4)n2)OCc2c(N4CCOCC4)ccnc2N3C(=O)OC(C)(C)C)C1]

Intermediate 642 was synthesized in a similar manner as Intermediate 86 using Intermediate 641 instead of Intermediate 85.

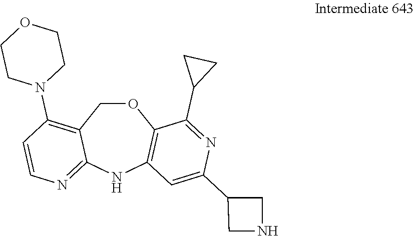 [SMILES: c1cc(N2CCOCC2)c2c(n1)Nc1cc(C3CNC3)nc(C3CC3)c1OC2]

Intermediate 643 was synthesized in a similar manner as Intermediate 87 using Intermediate 642 instead of Intermediate 86.

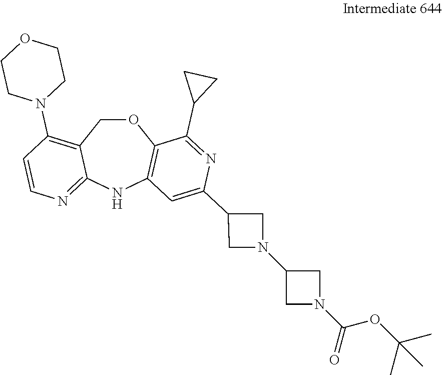 [SMILES: CC(C)(C)OC(=O)N1CC(N2CC(c3cc4c(c(C5CC5)n3)OCc3c(N5CCOCC5)ccnc3N4)C2)C1]

AcOH (0.15 mL, 2.56 mmol) was added to a solution of Intermediate 643 (809 mg, 2.13 mmol) and 1-Boc-3-azetidinone CAS [398489-26-4] (550 mg, 3.2 mmol) in MeOH (59 mL) and the reaction mixture was stirred at room temperature for 2 h. NaBH₃CN [CAS: 25895-60-7] (200 mg, 3.2 mmol) was then added and the mixture was stirred at room temperature for 16 h. To push the reaction to completion, more 1-Boc-3-azetidinone CAS [398489-26-4] (550 mg, 3.2 mmol) and NaBH₃CN [CAS: 25895-60-7] (200 mg, 3.2 mmol) were added, followed by an addition of AcOH (0.07 mL), and the reaction mixture was stirred at room temperature overnight. This last step was repeated 4 times, until Intermediate 643 was consumed. Saturated aqueous NaHCO₃ was added and the reaction mixture was extracted with EtOAc. The organic layer was washed with brine, dried over MgSO₄, filtered, and evaporated to dryness. Purification by flash column chromatography (gradient MeOH-DCM: 0% to 100%) to afford Intermediate 644 (732 mg, yield: 64%).

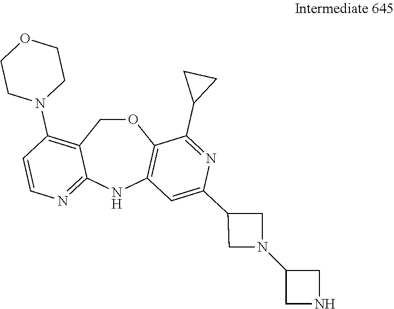 [SMILES: c1cc(N2CCOCC2)c2c(n1)Nc1cc(C3CN(C4CNC4)C3)nc(C3CC3)c1OC2]

TFA (5.6 mL) was added to a solution of Intermediate 644 (730 mg, 1.37 mmol) in DCM (8 mL) and the reaction mixture was stirred at room temperature for 3 h. The reaction mixture was evaporated to dryness to afford Intermediate 645 which was used in the next step without further purification.

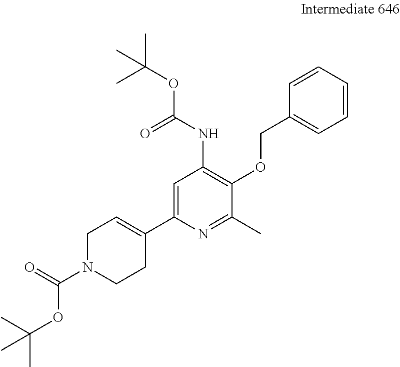 [SMILES: Cc1nc(C2=CCN(C(=O)OC(C)(C)C)CC2)cc(NC(=O)OC(C)(C)C)c1OCc1ccccc1]

Intermediate 646 was synthesized in a similar manner as Intermediate 549 using N-Boc-1,2,3,6-tetrahydropyridine-4-boronic acid pinacol ester [CAS: 286961-14-6]instead of tert-butyl 3-(4,4,5,5-tetramethyl-1,3,2-dioxaborolan-2-yl)-2,5-dihydro-TH-pyrrole-1-carboxylate (CAS: 212127-83-8).

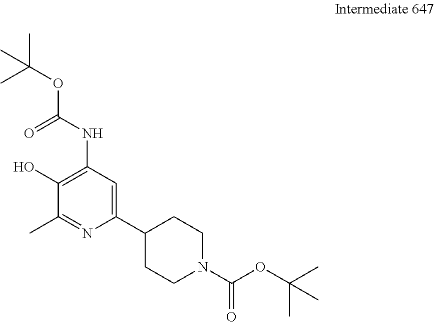 [SMILES: Cc1nc(C2CCN(C(=O)OC(C)(C)C)CC2)cc(NC(=O)OC(C)(C)C)c1O]

Intermediate 647 was synthesized in a similar manner as Intermediate 550 using Intermediate 646 instead of Intermediate 549.

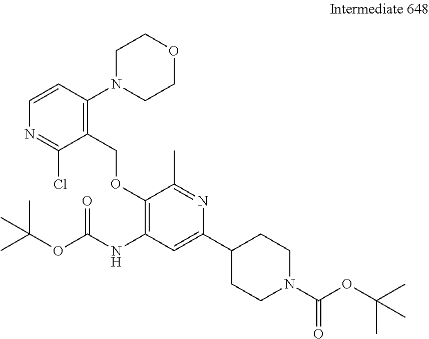 [SMILES: Cc1nc(C2CCN(C(=O)OC(C)(C)C)CC2)cc(NC(=O)OC(C)(C)C)c1OCc1c(N2CCOCC2)ccnc1Cl]

K₂CO₃ (2.37 g, 17.18 mmol, 2 eq.) was added to a solution of Intermediate 647 (3.5 g, 8.59 mmol) and Intermediate 60 (2.55 g, 10.31 mmol) in DMF (30 mL). The reaction mixture was stirred at room temperature for 18 h and then extracted with EtOAc. The organic layer was washed with brine, dried over MgSO₄, filtered, concentrated to dryness, and purified by column chromatography over silica gel (gradient MeOH in DCM) to afford Intermediate 648 (5.31 g, yield: 100%).

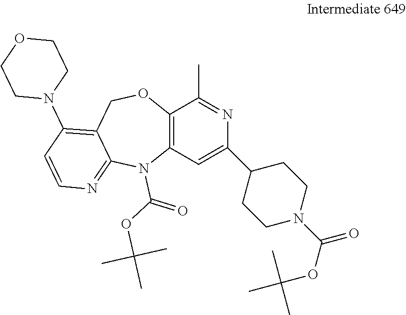 [SMILES: Cc1nc(C2CCN(C(=O)OC(C)(C)C)CC2)cc2c1OCc1c(N3CCOCC3)ccnc1N2C(=O)OC(C)(C)C]

Intermediate 649 was synthesized in a similar manner as Intermediate 629 using Intermediate 648 instead of Intermediate 628.

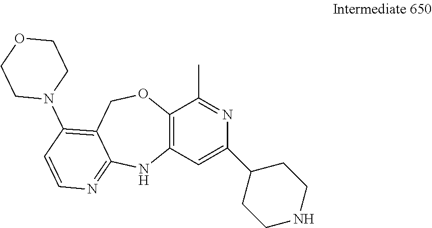 [SMILES: Cc1nc(C2CCNCC2)cc2c1OCc1c(N3CCOCC3)ccnc1N2]

TFA (16 mL) was added to a solution of Intermediate 649 (5.0 g, 8.59 mmol) in DCM (26 mL) and the reaction mixture was stirred at room temperature for 3 h. The reaction mixture was evaporated to dryness, then coevaporated with toluene twice to afford Intermediate 650 (10.13 g, yield: 100%).

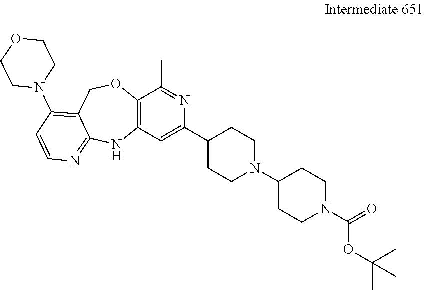 [SMILES: Cc1nc(C2CCN(C3CCN(C(=O)OC(C)(C)C)CC3)CC2)cc2c1OCc1c(N3CCOCC3)ccnc1N2]

tert-Butyl-4-oxopiperidine-1-carboxylate [CAS: 79099-07-3] (2.57 g, 12.88 mmol) was added to a solution of Intermediate 650 (10.13 g, 8.59 mmol) and Et₃N (8.36 mL, 60.12 mmol) in 1,2-DCE (100 mL) and the mixture was stirred for 1 h at room temperature. Sodium triacetoxyborohydride [CAS: 56553-60-7] (2.73 g, 12.88 mmol) was then added and the reaction mixture was stirred at room temperature overnight. Saturated aqueous NaHCO₃ was added and the reaction mixture was extracted with DCM. The combined organic layer was washed with brine, dried over MgSO₄, filtered, and concentrated to dryness. Purification by silica gel chromatography (gradient of heptane-EtOAc from 50% to 100% followed by a gradient of MeOH in DCM from 0% to 10%) afforded Intermediate 651 (2.94 g, yield: 61%).

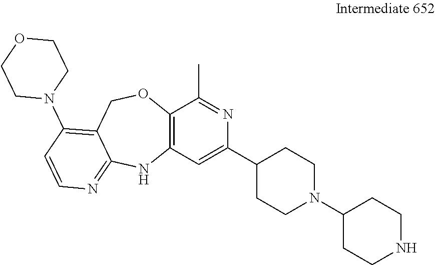 [SMILES: Cc1nc(C2CCN(C3CCNCC3)CC2)cc2c1OCc1c(N3CCOCC3)ccnc1N2]

TFA (10 mL) was added to a solution of Intermediate 651 (2.9 g, 5.14 mmol) in DCM (15 mL) and the reaction mixture was stirred at room temperature for 3 h. The reaction mixture was evaporated to dryness, then partitioned between DCM and a saturated aqueous solution of K₂CO₃. The organic layer was dried over MgSO₄, filtered, and concentrated to dryness to afford Intermediate 652 (2.39 g, yield: 100%).

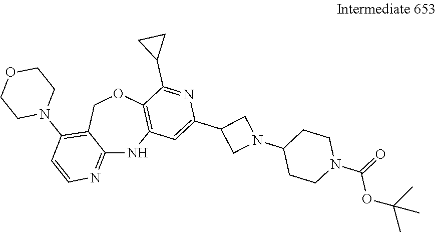 [SMILES: CC(C)(C)OC(=O)N1CCC(N2CC(c3cc4c(c(C5CC5)n3)OCc3c(N5CCOCC5)ccnc3N4)C2)CC1]

tert-Butyl-4-oxopiperidine-1-carboxylate [CAS: 79099-07-3] (1.25 g, 6.29 mmol) was added to a solution of Intermediate 643 (4.94 g, 4.19 mmol) and Et₃N (3.5 mL, 25.15 mmol) in 1,2-DCE (56 mL) and the mixture was stirred for 1 h at room temperature. Sodium triacetoxyborohydride [CAS: 56553-60-7] (1.33 g, 6.29 mmol) was then added and the reaction mixture was stirred at room temperature for 18 h. Saturated aqueous NaHCO₃ was added and the reaction mixture was extracted with DCM. The combined organic layer was washed with brine, dried over MgSO₄, filtered, and concentrated to dryness. Purification by silica gel chromatography (gradient of MeOH in DCM) afforded Intermediate 653 (2.24 g, yield: 95%).

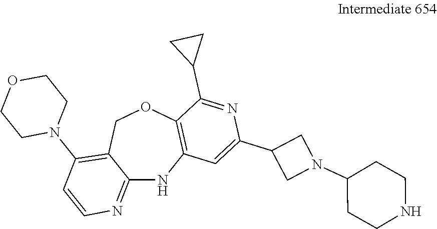 [SMILES: c1cc(N2CCOCC2)c2c(n1)Nc1cc(C3CN(C4CCNCC4)C3)nc(C3CC3)c1OC2]

TFA (16 mL) was added to a solution of Intermediate 653 (2.24 g, 3.98 mmol) in DCM (24 mL) and the reaction mixture was stirred at room temperature for 3 h. The reaction mixture was evaporated to dryness to afford Intermediate 654 (1.84 g, quantitative yield), used without further purification.

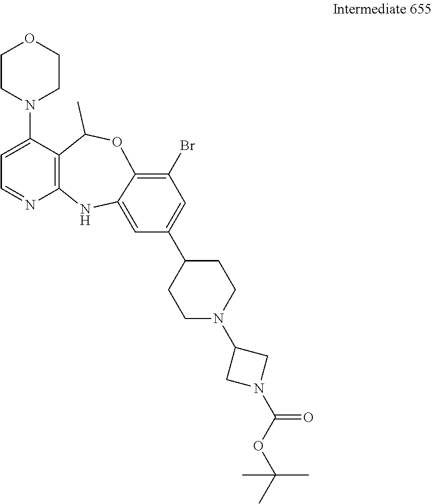 [SMILES: CC1Oc2c(Br)cc(C3CCN(C4CN(C(=O)OC(C)(C)C)C4)CC3)cc2Nc2nccc(N3CCOCC3)c21]

Sodium triacetoxyborohydride (446 mg, 2.104 mmol, 2 eq.) was added to a stirred mixture of Intermediate 492 (476 mg 1.036 mmol), 1-Boc-3-azetidinone (CAS [398489-26-4], 272 mg, 1.589 mmol, 1.5 eq.), and AcOH (100 μL, 1.758 mmol, 1.7 eq.) in DCM (5.5 mL). The reaction mixture was stirred at room temperature for 16 h. The mixture was diluted with saturated aqueous Na₂CO₃ and extracted with DCM. The organic layer was separated, washed with water, then with brine, dried (Na₂SO₄), filtered, and concentrated in vacuo. The residue was purified by flash column chromatography (silica; EtOAc/heptane 30/70 to 100/0) to yield Intermediate 655 (636 mg, quantitative) as a white solid.

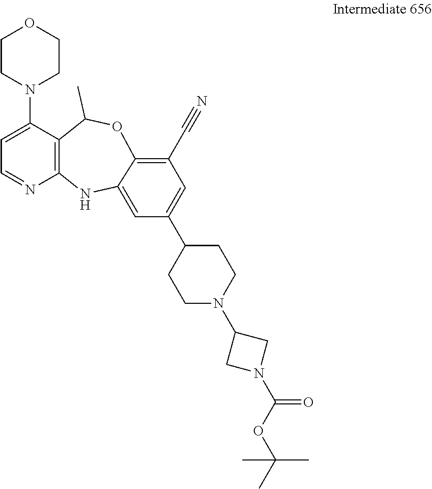 [SMILES: CC1Oc2c(C#N)cc(C3CCN(C4CN(C(=O)OC(C)(C)C)C4)CC3)cc2Nc2nccc(N3CCOCC3)c21]

In a sealed tube (microwave), a mixture of Intermediate 655 (733 mg, 1.193 mmol), XPhos Pd G3 (CAS [1445085-55-1], 105 mg, 0.124 mmol, 0.1 eq.), tBu XPhos (CAS [564483-19-8], 65 mg, 0.153 mmol, 0.1 eq.), potassium hexacyanoferrate(II) trihydrate (CAS [14459-95-1], 353 mg, 0.836 mmol, 0.7 eq.), and KOAc (65 mg, 0.662 mmol, 0.5 eq.) in 1,4-dioxane (6.5 mL) and water (6.5 mL) was purged under nitrogen flux. The vial was sealed and the reaction mixture was stirred at 100° C. for 1 h. After cooling, the reaction mixture was poured into aqueous K₂CO₃ (10%). This mixture was extracted twice with DCM. The organic layer was decanted on Chromabond® and the solvent was evaporated. The residue was taken up in ACN, partially dissolved, and a few drops of Et₂O were added. The precipitate that appeared was filtered and dried to give a first fraction of Intermediate 656 (149 mg, yield: 22%). The filtrate was concentrated and the residue was purified by column chromatography (Irregular SiO₂ 15-40 μm 40 g GraceResolv®, gradient from: 98% DCM, 2% MeOH, 0.2% NH₄OH to 94% DCM, 6% MeOH, 0.6% NH₄OH) to give a second batch of Intermediate 656 (377 mg, yield: 56%)

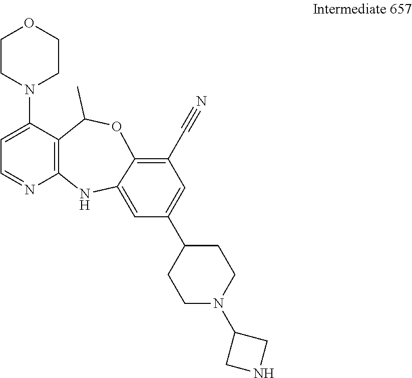 [SMILES: CC1Oc2c(C#N)cc(C3CCN(C4CNC4)CC3)cc2Nc2nccc(N3CCOCC3)c21]

TFA (1.3 mL, 16.988 mmol, 18 eq.) was added to a solution of Intermediate 656 (526 mg, 0.938 mmol) in DCM (15 mL) at 0° C. The reaction mixture was stirred at room temperature overnight. The volatiles were evaporated. The residue was basified with aqueous NH₄OH (30%) and extracted twice with DCM. The organic layer was separated and the solvent was evaporated to give Intermediate 657 (360 mg, yield: 83%).

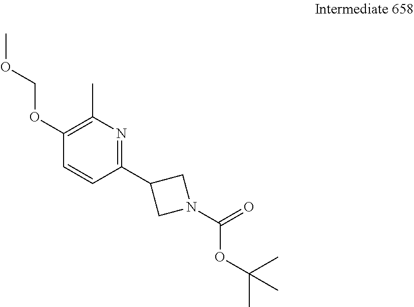 [SMILES: COCOc1ccc(C2CN(C(=O)OC(C)(C)C)C2)nc1C]

A mixture of 6-bromo-3-(methoxymethoxy)-2-methyl-pyridine (CAS [1783265-24-6], 24 g, 103.415 mmol), 1-t-butoxycarbonylazetidin-3-yl zinc iodide (CAS [206446-38-0], 0.62 M in DMA, 250 mL, 155.122 mmol, 1.5 eq.), PdCl₂(dppf). DCM (CAS [95464-05-4], 4.2 g, 5.171 mmol, 0.05 eq.), and CuI (1.97 g, 10.341 mmol, 0.1 eq.) in DMA (250 mL) was stirred at 80° C. for 1 h under nitrogen atmosphere. EtOAc (500 mL) was added and the mixture was washed with water (5×300 mL). The organic layer was dried over MgSO₄, filtered, and evaporated. The residue was purified by column chromatography over silica gel (eluent: petroleum ether/EtOAc 100:0 to 70:30) to give Intermediate 658 (52 g, yield: 83%).

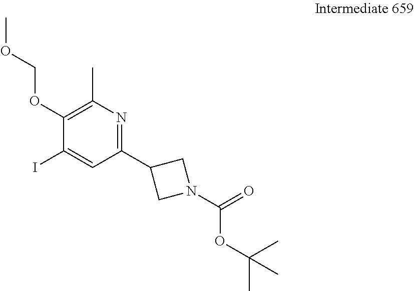 [SMILES: COCOc1c(I)cc(C2CN(C(=O)OC(C)(C)C)C2)nc1C]

BuLi (2.5 M in THF, 68 mL, 170.093 mmol, 1.2 eq.) was added dropwise over 15 min to a solution of Intermediate 658 (47 g, 141.744 mmol) in TIF (400 mL) at −78° C. under nitrogen atmosphere. The reaction mixture was stirred at −78° C. for 15 min. A solution of iodine (43 g, 170.093 mmol, 1.2 eq.) in THF (200 mL) was added dropwise over 15 min at −78° C. under nitrogen atmosphere and the mixture was stirred at −78° C. for 1 h. Saturated aqueous NH₄Cl (200 mL) and EtOAc (700 mL) were added. The mixture was separated and the organic layer was washed with saturated aqueous Na₂S₂O₃ (2×400 mL). The organic layer was dried over MgSO₄, filtered, and evaporated. The residue was purified by column chromatography over silica gel (eluent: petroleum ether/EtOAc 100:0 to 50:50) to afford Intermediate 659 (58 g, yield: 85%) as a yellow oil.

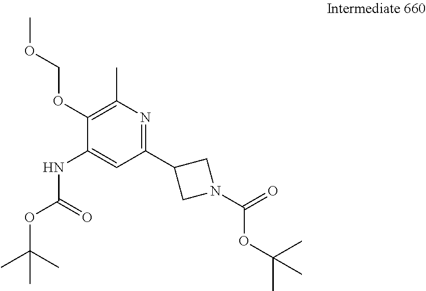 [SMILES: COCOc1c(NC(=O)OC(C)(C)C)cc(C2CN(C(=O)OC(C)(C)C)C2)nc1C]

A solution of Intermediate 659 (53 g, 122.044 mmol), t-butylcarbamate (CAS [4248-19-5], 17.1 g, 146.453 mmol, 1.2 eq.), Pd₂(dba)₃ (CAS [51364-51-3], 3.35 g, 3.661 mmol, 0.03 eq.), Xantphos (CAS [161265-03-8], 4.24 g, 7.323 mmol, 0.06 eq.), and Cs₂C03 (79.5 g, 244.088 mmol, 2 eq.) in toluene (300 mL) was stirred at 100° C. under nitrogen atmosphere overnight. After cooling, the reaction mixture was filtered. The filtrate was diluted with EtOAc (800 mL) and washed with water (3×300 mL). The organic layer was dried over MgSO₄, filtered, and evaporated. The residue was purified by column chromatography over silica gel (eluent: petroleum ether/EtOAc 100:0 to 0:100) to afford Intermediate 660 (53 g, yield: 94%) as a yellow oil.

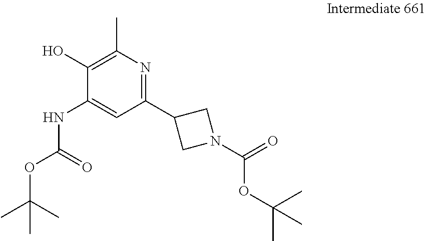 [SMILES: Cc1nc(C2CN(C(=O)OC(C)(C)C)C2)cc(NC(=O)OC(C)(C)C)c1O]

HCl (37% in water, 1.9 mL, 22.668 mmol, 1.2 eq.) was added dropwise to a solution of Intermediate 660 (8 g, 18.89 mmol) in iPrOH (100 mL) at room temperature. The reaction mixture was stirred at room temperature for 3 days. Saturated aqueous NaHCO₃(100 mL) followed by water were added and the mixture was extracted with EtOAc (3×600 mL). The organic layer was dried over MgSO₄, filtered, and evaporated. The residue was purified by column chromatography over silica gel (eluent: DCM/MeOH 100:0 to 80:20) to afford Intermediate 661 (35.1 g, yield: 87%) as a yellow solid.

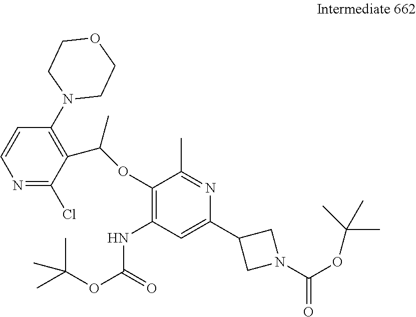 [SMILES: Cc1nc(C2CN(C(=O)OC(C)(C)C)C2)cc(NC(=O)OC(C)(C)C)c1OC(C)c1c(N2CCOCC2)ccnc1Cl]

Intermediate 27 (8.6 g 32.932 mmol, 1.1 eq.) was added to a solution of Intermediate 661 (11.36 g, 29.938 mmol) and K₂CO₃ (8.275 g, 59.875 mmol, 2 eq.) in dry DMF (75 mL). The reaction mixture was stirred at room temperature for 16 h. EtOAc and brine were added to the reaction mixture and the layers were separated. The organic layer was dried over MgSO₄, filtered, and concentrated. The residue was purified by column chromatography (Stationary phase: irregular SiOH 35-70 μm 330 g, Mobile phase: gradient from 100% DCM to 95% DCM, 5% MeOH, 0.5% NH₄OH) to give Intermediate 662 (7.8 g, yield: 43%).

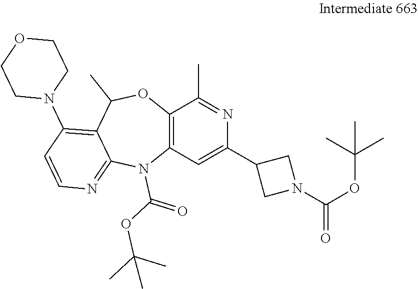 [SMILES: Cc1nc(C2CN(C(=O)OC(C)(C)C)C2)cc2c1OC(C)c1c(N3CCOCC3)ccnc1N2C(=O)OC(C)(C)C]

A mixture of Intermediate 662 (4.7 g, 7.78 mmol), bis(dibenzylideneacetone)palladium (CAS [32005-36-0], 447 mg, 0.778 mmol, 0.1 eq.), rac-bis(diphenylphosphino)-1,1′-binaphthyl (CAS [98327-87-8], 484 mg, 0.778 mmol, 0.1 eq.), and sodium tert-butoxide (7.39 mL, 2 M in Me-THF, 14.781 mmol, 1.9 eq.) in dry toluene (35 mL) was stirred at 100° C. for 6 h. After cooling, the mixture was poured into ice/water. EtOAc was added and the mixture was filtered through a layer of celite. The celite was washed with EtOAc and the filtrate was extracted with EtOAc. The organic layer was washed with brine, dried over MgSO₄, filtered, and the solvent was evaporated. The residue was purified by column chromatography (Stationary phase: irregular SiOH 35-70 pam 330 g, Mobile phase: gradient from 97% DCM, 3% MeOH, 0.3% NH₄OH to 85% DCM, 14% MeOH, 1.4% NH₄OH) to give Intermediate 663 (4.17 g, yield: 94%).

### Intermediate 664A and Intermediate 664B

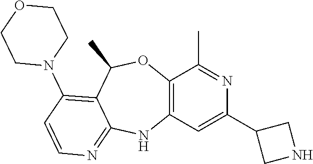 [SMILES: Cc1nc(C2CNC2)cc2c1O[C@H](C)c1c(N3CCOCC3)ccnc1N2]

Intermediate 664A: (*R), Pure stereoisomer but absolute stereochemistry undetermined

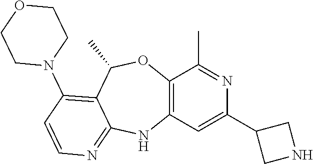 [SMILES: Cc1nc(C2CNC2)cc2c1O[C@@H](C)c1c(N3CCOCC3)ccnc1N2]

Intermediate 664B: (*S), Pure stereoisomer but absolute stereochemistry undetermined TFA (34.4 mL, 449.132 mmol, 100 eq.) was added to a solution of Intermediate 663 (2.1 g, 4.491 mmol) in DCM (89 mL) at room temperature. The reaction mixture was stirred at room temperature for 12 h. The solvent was evaporated and the residue was poured onto ice. Water and NH₄OH were added until basic pH. The mixture was extracted twice with DCM. The organic layer was decanted on Chromabond® and the solvent was evaporated. The residue was purified by chiral SFC (Stationary phase: CHIRALPAK AD-H 5 μm 250*30 mm, Mobile phase: 45% CO₂, 55% EtOH (0.3% iPrNH₂)) to give Intermediate 664A (363 mg, yield: 22%) and its enantiomer Intermediate 664B (364 mg, yield: 22%).

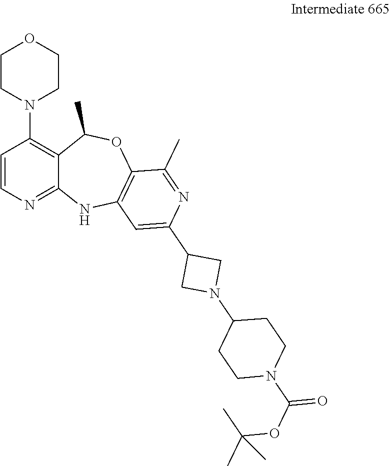 [SMILES: Cc1nc(C2CN(C3CCN(C(=O)OC(C)(C)C)CC3)C2)cc2c1O[C@H](C)c1c(N3CCOCC3)ccnc1N2]

(*R), Pure stereoisomer but absolute stereochemistry undetermined 1-Boc-4-piperidone (CAS [79099-07-3], 484 mg, 2.429 mmol, 2 eq.) and AcOH (70 μL, 1.214 mmol, 1 eq.) were added to a solution of Intermediate 664A (460 mg, 1.214 mmol) in DCE (15 mL) and the reaction mixture was stirred at room temperature for 30 min. Sodium triacetoxyborohydride (386 mg, 1.821 mmol, 1.5 eq.) was added portionwise and the mixture was stirred at room temperature for 5 h. DCM and 1 M aqueous Na₂CO₃ were added and the layers were separated. The organic layer was dried over MgSO₄, filtered, and concentrated. The residue was purified by column chromatography over silica gel (DCM/MeOH (9:1) in DCM from 0% to 60%) to afford Intermediate 665 (449 mg, yield: 65%) as an oil.

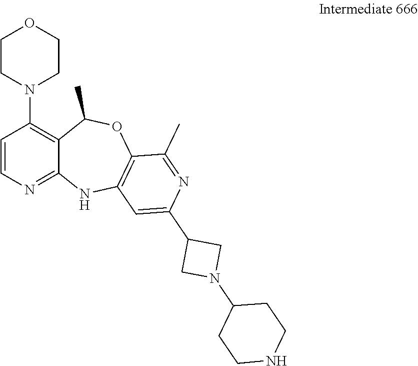 [SMILES: Cc1nc(C2CN(C3CCNCC3)C2)cc2c1O[C@H](C)c1c(N3CCOCC3)ccnc1N2]

(*R), pure stereoisomer but absolute stereochemistry undetermined TFA (624 μL, 8.153 mmol, 10 eq.) was added to a solution of Intermediate 665 (4498 mg, 0.815 mmol) in DCM (10 mL). The reaction mixture was stirred overnight at room temperature. Volatiles were evaporated and the residue was washed with toluene twice. The residue was treated with Amberlyst A26 hydroxide until pH 7. The resin was filtered off and washed successively with MeOH (25 mL) and DCM (25 mL). The filtrate was evaporated to afford Intermediate 666 (356 mg, yield: 95%) as an oil, used without further purification.

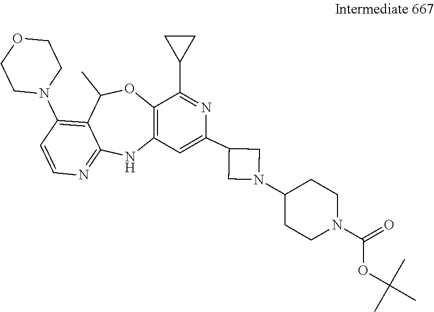 [SMILES: CC1Oc2c(cc(C3CN(C4CCN(C(=O)OC(C)(C)C)CC4)C3)nc2C2CC2)Nc2nccc(N3CCOCC3)c21]

tert-Butyl-4-oxopiperidine-1-carboxylate (CAS [79099-07-3], 1.275 g, 6.4 mmol, 2 eq.) was added to a solution of Intermediate 633 (3.448 g, 3.2 mmol) and Et₃N (3.1 mL, 22.4 mmol, 7 eq.) in DCE (60 mL), and the reaction mixture was stirred at room temperature for 1 h. Sodium triacetoxyborohydride (1.356 g, 6.4 mmol, 2 eq.) was then added and the mixture was stirred at room temperature for 18 h. Aqueous NaHCO₃ was added to the reaction mixture the mixture was extracted with DCM. The organic layer was washed with brine, dried over MgSO₄ filtered, and evaporated. The residue was purified by flash column chromatography on silica gel (gradient MeOH in DCM 0% to 10%) to give Intermediate 667 (1.635 g, yield: 89%).

### Intermediate 668A and Intermediate 668B

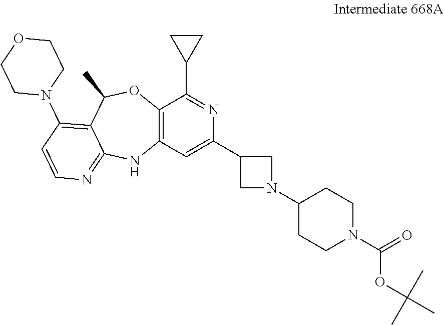 [SMILES: C[C@H]1Oc2c(cc(C3CN(C4CCN(C(=O)OC(C)(C)C)CC4)C3)nc2C2CC2)Nc2nccc(N3CCOCC3)c21] [1022] (*R), pure stereoisomer but absolute stereochemistry undetermined

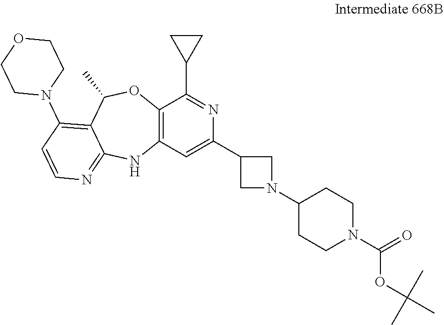 [SMILES: C[C@@H]1Oc2c(cc(C3CN(C4CCN(C(=O)OC(C)(C)C)CC4)C3)nc2C2CC2)Nc2nccc(N3CCOCC3)c21]

(*S), pure stereoisomer but absolute stereochemistry undetermined Intermediate 667 was separated into its enantiomers by chiral SFC (Amilose-1, isocratic mode 55% CO₂− 45% EtOH) to afford Intermediate 668A and Intermediate 668B.

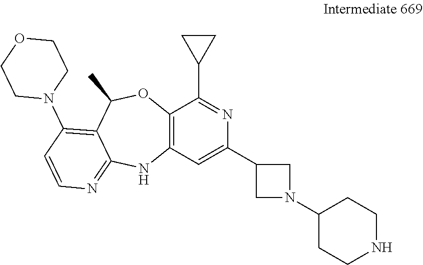 [SMILES: C[C@H]1Oc2c(cc(C3CN(C4CCNCC4)C3)nc2C2CC2)Nc2nccc(N3CCOCC3)c21]

(*R), pure stereoisomer but absolute stereochemistry undetermined TFA (8 mL) was added to a solution of Intermediate 668A (761 mg, 1.32 mmol) in DCM (12 mL) at room temperature. The reaction mixture was stirred at room temperature for 3 h. The volatiles were evaporated to give Intermediate 669 (629 mg, quantitative), used without further purification.

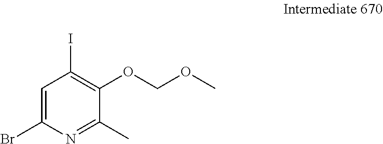

Sodium hydride (60% in mineral oil, 9.97 g, 249.237 mmol, 1.2 eq.) was added to a solution of 6-bromo-4-iodo-2-methyl-3-pyridinol (65.2 g, 207.698 mmol) in DMF (450 mL) at 0° C. under nitrogen atmosphere. The reaction mixture was stirred for 10 min, before MOMCl (CAS [107-30-2], 20.51 mL, 270.007 mmol, 1.3 eq.) was added dropwise. Stirring was continued for 6 h at room temperature. The reaction was quenched with water. Brine was added and organics were extracted with EtOAc. The organic layer was dried over MgSO₄, filtered, and concentrated. The residue was purified by flash column chromatography (silica; heptane/EtOAc gradient) to afford Intermediate 670 (56.3 g, yield: 76%) as a white solid.

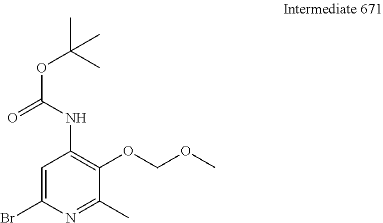 [SMILES: COCOc1c(NC(=O)OC(C)(C)C)cc(Br)nc1C]

Pd(OAc)₂ (1.765 g, 7.864 mmol, 0.05 eq.) followed by tert-butyl carbamate (CAS[4248-19-5], 12.897 g, 110.093 mmol, 0.7 eq.) were added to a suspension of Intermediate 670 (56.3 g, 157.275 mmol), Xantphos (CAS [161265-03-8], 4.55 g, 7.864 mmol, 0.05 eq.), and Cs₂CO₃ (102.487 g, 314.551 mmol, 2 eq.) in toluene (300 mL) under nitrogen atmosphere. The mixture was degassed by bubbling nitrogen for 15 min and was then stirred at 50° C. for 16 h. After cooling, the reaction mixture was diluted with water and extracted with EtOAc. The organic layer was dried over MgSO₄, filtered, and concentrated. The residue was purified by flash column chromatography (silica, heptane/EtOAc gradient) to afford Intermediate 671 (25.6 g, yield: 47%).

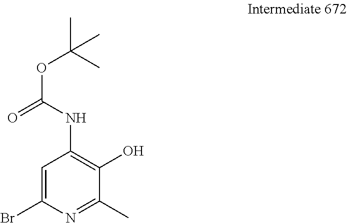 [SMILES: Cc1nc(Br)cc(NC(=O)OC(C)(C)C)c1O]

HCl (37% in water, 6.8 mL, 81.105 mmol, 1.1 eq.) was added to a solution of Intermediate 671 (25.6 g, 73.732 mmol) in iPrOH (300 mL). The reaction mixture was stirred at room temperature overnight. Saturated aqueous NaHCO₃ was added until pH 7. The organics were extracted with DCM. The organic layer was washed with brine, dried over MgSO₄, filtered, and concentrated to afford Intermediate 672 (22 g, yield: 98%), used without further purification.

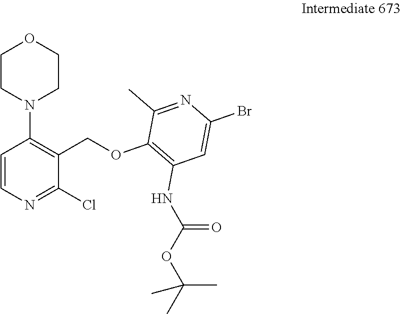 [SMILES: Cc1nc(Br)cc(NC(=O)OC(C)(C)C)c1OCc1c(N2CCOCC2)ccnc1Cl]

Intermediate 672 (10.55 g, 34.791 mmol) was added to a mixture of Intermediate 60 (9.46 g, 38.270 mmol, 1.1 eq.) and K₂CO₃ (9.62 g, 69.582 mmol, 2 eq.) in DMF (150 mL). The reaction mixture was stirred at room temperature overnight. Water and DCM were added and the layers were separated. The organic layer was dried over MgSO₄, filtered, and concentrated. The residue was purified by column chromatography over silica gel (gradient EtOAc in heptane from 0% to 80%) to give Intermediate 673 (19.02 g, quantitative) as an orange gum.

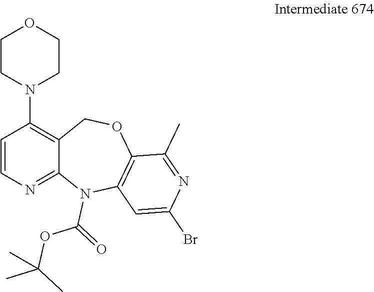 [SMILES: Cc1nc(Br)cc2c1OCc1c(N3CCOCC3)ccnc1N2C(=O)OC(C)(C)C]

A mixture of Intermediate 673 (19.02 g, 32.570 mmol), Cs₂CO₃ (15.92 g, 48.855 mmol, 1.5 eq.), S-Phos (CAS [657408-07-6], 1.00 g, 2.443 mmol, 0.075 eq.), and Pd(OAc)₂ (0.55 g, 2.443 mmol, 0.075 eq.) in toluene (300 mL) was degassed by bubbling nitrogen through the solution. The reaction mixture was stirred at 105° C. overnight. After cooling, water (75 mL) and -EtOAc (150 mL) were added and the layers were separated. The aqueous layer was extracted again with EtOAc (50 mL). The combined organic layers were dried over MgSO₄, filtered, and concentrated in vacuo. The residue was purified by flash column chromatography (silica; EtOAc in heptane 0/100 to 100/0) to give Intermediate 674 (10.96 g, yield: 56%) as an off-white foam.

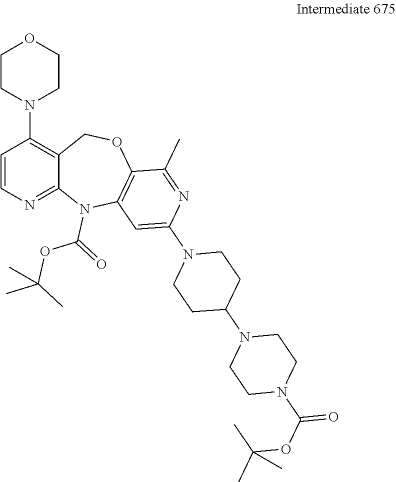 [SMILES: Cc1nc(N2CCC(N3CCN(C(=O)OC(C)(C)C)CC3)CC2)cc2c1OCc1c(N3CCOCC3)ccnc1N2C(=O)OC(C)(C)C]

Intermediate 674 (500 mg, 1.047 mmol), 1,1-dimethylethyl 4-(4-piperidinyl)-1-piperazinecarboxylate (CAS [205059-24-1], 339 mg, 1.257 mmol, 1.2 eq.), and Cs₂CO₃ (683 mg, 2.095 mmol, 2 eq.) were suspended in toluene (10 mL) and the mixture was degassed with nitrogen for 15 min. S-Phos (CAS [657408-07-6], 43 mg, 0.105 mmol, 0.1 eq.) and Pd₂(dba)₃ (CAS [51364-51-3], 96 mg, 0.105 mmol, 0.1 eq.) were added and the resulting mixture was stirred at 100° C. overnight. To push the reaction to completion, more S-Phos (CAS [657408-07-6], 43 mg, 0.105 mmol. 0.1 eq.) and Pd₂(dba)₃ (CAS [51364-51-3], 96 mg, 0.105 mmol, 0.1 eq.) were added and the reaction mixture was stirred at 100° C. for 24 h. Brine and EtOAc were added and the layers were separated. The organic layer was dried over MgSO₄, filtered, and concentrated. The residue was purified by flash column chromatography (silica gel, [DCM/MeOH 9:1]/DCM, from 0/100 to 55/45) to afford Intermediate 675 (215 mg, yield: 26%) as an oil.

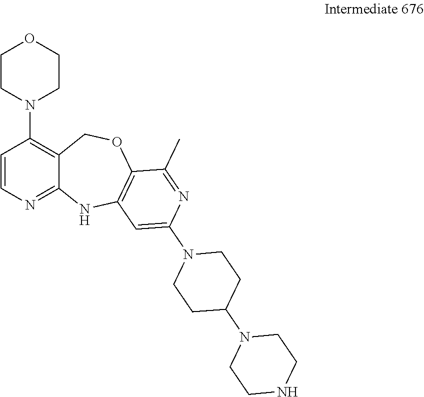 [SMILES: Cc1nc(N2CCC(N3CCNCC3)CC2)cc2c1OCc1c(N3CCOCC3)ccnc1N2]

TFA (1.66 mL, 21.675 mmol, 20 eq.) was added to solution of Intermediate 675 (820 mg, 1.084 mmol) in DCM (15 mL). The reaction mixture was stirred overnight at room temperature. The mixture was concentrated and the residue was washed with toluene twice before it was treated with Amberlyst A26 hydroxide until pH 7. The resin was filtered off and washed successively with MeOH (75 mL) and DCM (75 mL). The filtrate was evaporated to afford Intermediate 676 (505 mg, yield: 95%), used without further purification.

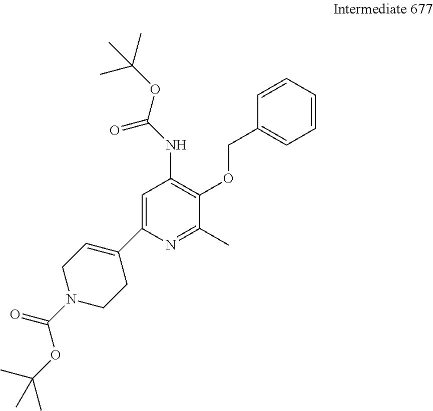 [SMILES: Cc1nc(C2=CCN(C(=O)OC(C)(C)C)CC2)cc(NC(=O)OC(C)(C)C)c1OCc1ccccc1]

Pd(dppf)Cl₂·DCM (CAS[95464-05-41] 812 mg, 0.992 mmol, 0.05 eq.) was added to a suspension of Intermediate 548 (7.8 g, 19.833 mmol), N-Boc-1,2,3,6-tetrahydropyridine-4-boronic acid pinacol ester (CAS [286961-14-6], 7.359 g, 23.8 mmol, 1.2 eq.), and K₃PO₄ (8.42 g, 39.667 mmol, 2 eq.) in a mixture of 1,4-dioxane (70 mL) and water (12 mL), under nitrogen atmosphere. The reaction mixture was stirred overnight at 80° C. under nitrogen atmosphere. The reaction mixture was partitioned between EtOAc and brine. The organic layer was concentrated and the residue was purified by column chromatography on silica gel (heptane/EtOAc gradient) to afford Intermediate 677 (7.37 g, yield: 75%).

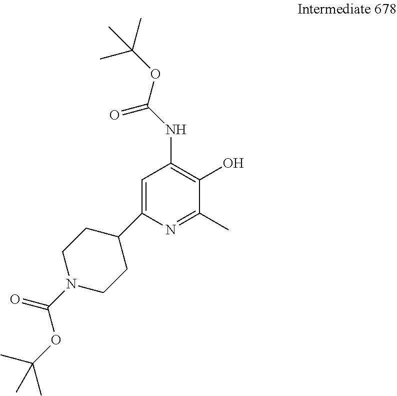 [SMILES: Cc1nc(C2CCN(C(=O)OC(C)(C)C)CC2)cc(NC(=O)OC(C)(C)C)c1O]

Pd/C (10%, 500 mg) was added to a solution of Intermediate 677 (33.9 g, 68.401 mmol) in a mixture of MeOH (320 mL) and THF (110 mL) under nitrogen atmosphere. The reaction mixture was purged with hydrogen and was stirred overnight at room temperature under hydrogen atmosphere (1 atm). The mixture was filtered over a pad of celite, and the solvent was removed under reduced pressure to give Intermediate 678 (27.874 g, quantitative), used without further purification.

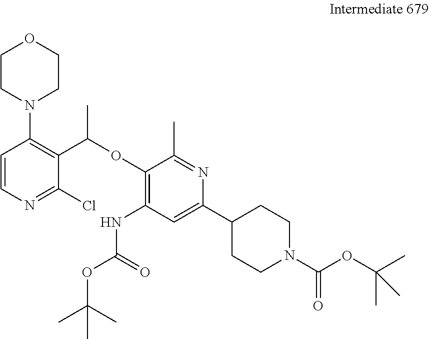 [SMILES: Cc1nc(C2CCN(C(=O)OC(C)(C)C)CC2)cc(NC(=O)OC(C)(C)C)c1OC(C)c1c(N2CCOCC2)ccnc1Cl]

K₂CO₃ (8.14 g, 58.895 mmol, 3 eq.) was added to a suspension of Intermediate 678 (8 g, 19.632 mmol) in DMF (48 mL). The reaction mixture was stirred at room temperature while Intermediate 27 (10.254 g, 39.263 mmol, 2 eq.) was added in small portions over 5 h. After the addition, the reaction mixture was stirred at room temperature for 16 h To push the reaction to completion, additional Intermediate 27 (7.69 g, 29.447 mmol, 1.5 eq.) was added and the mixture was stirred for a further 16 h. The mixture was diluted with EtOAc and washed with brine (5×). The organic layer was evaporated and the residue was purified by flash column chromatography on silica gel (DCM-MeOH gradient) to give Intermediate 679 (12.411 g, quantitative).

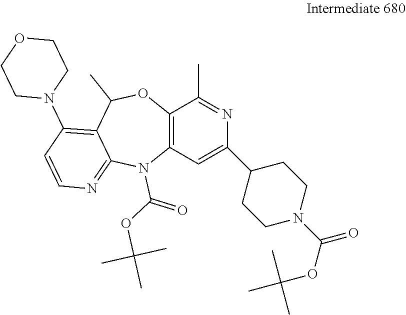 [SMILES: Cc1nc(C2CCN(C(=O)OC(C)(C)C)CC2)cc2c1OC(C)c1c(N3CCOCC3)ccnc1N2C(=O)OC(C)(C)C]

A solution of Intermediate 679 (5.617 g, 8.885 mmol) and Cs₂CO₃ (4.342 g, 13.327 mmol, 1.5 eq.) in toluene (100 mL) was degassed with nitrogen. Xantphos (CAS [161265-03-8], 771 mg, 1.333 mmol, 0.15 eq.) and Pd(OAc)₂ (299 mg, 1.333 mmol, 0.15 eq.) were added. The reaction mixture was degassed again with nitrogen and stirred at 100° C. for 72 h. The reaction mixture was partitioned between EtOAc and brine. The organic layer was concentrated and the residue was purified by flash column chromatography (SiO₂, EtOAc-heptane) to afford Intermediate 680 (4.475 g, yield: 85%).

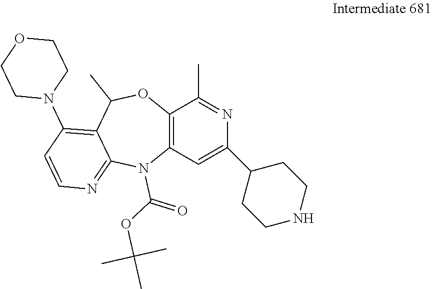 [SMILES: Cc1nc(C2CCNCC2)cc2c1OC(C)c1c(N3CCOCC3)ccnc1N2C(=O)OC(C)(C)C]

TFA (30 mL) was added to Intermediate 680 (4.475 g, 7.512 mmol) in DCM (45 mL) at room temperature and the reaction mixture was stirred at room temperature for 3 h. The volatiles were evaporated and the residue was co-evaporated with toluene (2×100 mL) to give Intermediate 681 (TFA salt, 8.96 g, quantitative) after drying under vacuum.

### Intermediate 682, Intermediate 683, and Intermediate 684

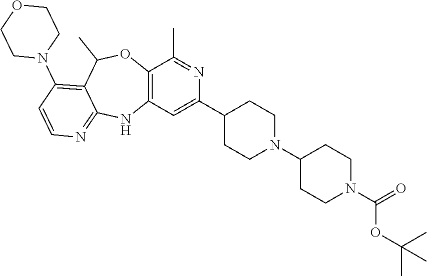 [SMILES: Cc1nc(C2CCN(C3CCN(C(=O)OC(C)(C)C)CC3)CC2)cc2c1OC(C)c1c(N3CCOCC3)ccnc1N2] [1038] Intermediate 682: mixture of stereoisomers

Intermediate 683: (*S), pure stereoisomer but absolute stereochemistry undetermined

Intermediate 684: (*R), pure stereoisomer but absolute stereochemistry undetermined tert-Butyl-4-oxopiperidine-1-carboxylate (CAS [79099-07-3], 2.245 g, 11.268 mmol, 1.5 eq.) was added to a solution of Intermediate 681 (8.967 g, 7.512 mmol) and Et₃N (7.31 mL, 52.584 mmol, 7 eq.) in DCE (100 mL) and the reaction mixture was stirred at room temperature for 1 h. Sodium triacetoxyborohydride (2.388 g, 11.268 mmol, 1.5 eq.) was then added and the mixture was stirred at room temperature for 18 h. To push the reaction to completion, more tert-butyl-4-oxopiperidine-1-carboxylate (2.245 g, 11.268 mmol, 1.5 eq.) was added and the reaction mixture was stirred at room temperature for 1 h. Sodium triacetoxyborohydride (2.388 g, 11.268 mmol, 1.5 eq.) was added and the reaction mixture was stirred overnight. Aqueous NaHCO₃ was added and the mixture was extracted with DCM. The organic layer was washed with brine, dried over MgSO₄, filtered, and evaporated. The residue was purified by flash column chromatography (MeOH-DCM) to afford Intermediate 682 (3.988 g, yield: 92%). Intermediate 682 was separated into its enantiomers by chiral HPLC (Phenomenex-LuxAmylose-1 (150×4.6 mm, 5 μm); eluent: iPrOH 40% −EtOH 60% isocratic) to give Intermediate 683 (1.455 g, yield: 37%) and Intermediate 684 (2.220 g, yield: 56%).

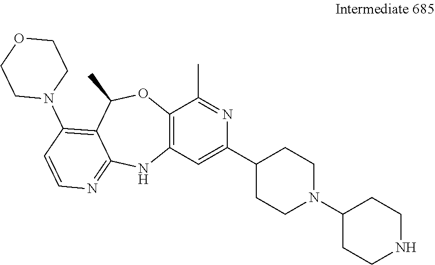 [SMILES: Cc1nc(C2CCN(C3CCNCC3)CC2)cc2c1O[C@H](C)c1c(N3CCOCC3)ccnc1N2]

(*R) pure stereoisomer but absolute stereochemistry undetermined TFA (8.88 mL) was added to a solution of Intermediate 684 (2.22 g, 3.836 mmol) at room temperature and the reaction mixture was stirred at room temperature for 3 h. The volatiles were evaporated to give Intermediate 685 (1.836 g, quantitative), used without further purification.

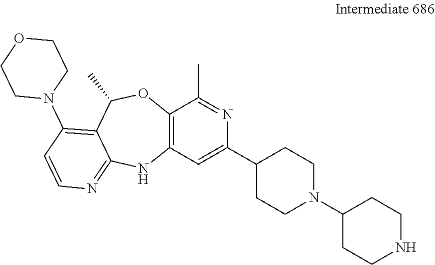 [SMILES: Cc1nc(C2CCN(C3CCNCC3)CC2)cc2c1O[C@@H](C)c1c(N3CCOCC3)ccnc1N2]

(*S), pure stereoisomer but absolute stereochemistry undetermined Intermediate 686 was prepared following the same procedure as Intermediate 685, starting from Intermediate 683 instead of Intermediate 684.

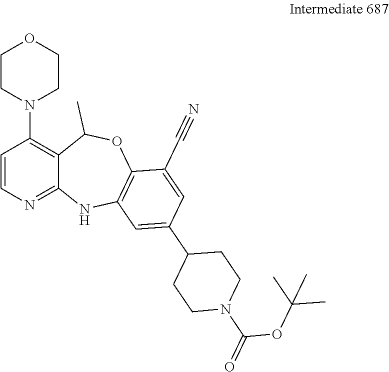 [SMILES: CC1Oc2c(C#N)cc(C3CCN(C(=O)OC(C)(C)C)CC3)cc2Nc2nccc(N3CCOCC3)c21]

In a sealed vessel, a solution of Intermediate 621 (2.78 g, 4.97 mmol), potassium hexacyanoferrate(II) trihydrate (1.05 g, 2.48 mmol, 0.5 eq.), and KOAc (244 mg, 2.48 mmol, 0.5 eq.) in 1,4-dioxane (22 mL) and water (22 mL) was purged with nitrogen. Xphos Pd G3 (CAS [1445085-55-1], 421 mg, 0.497 mmol, 0.1 eq.) and tBu XPhos (CAS [564483-19-8], 211 mg, 0.497 mmol, 0.1 eq.) were added. The reaction mixture was purged again with nitrogen and stirred at 100° C. for 1 h. The mixture was poured in water and EtOAc. The organic layer was separated and washed with brine, dried over MgSO₄, filtered, and the solvent was evaporated. The residue was combined with another batch and was purified by chromatography over silica gel (SiO₂, 120 g, eluent: from 100% DCM to 95% DCM, 5% MeOH, 0.5% NH₄OH) to give Intermediate 687 (3.5 g, yield for the combined batch: 81%) as an off-white foam

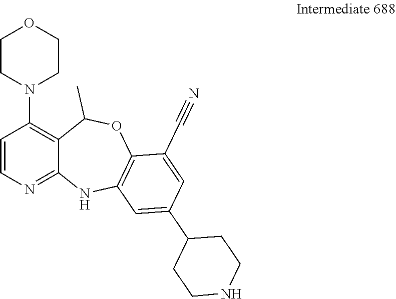 [SMILES: CC1Oc2c(C#N)cc(C3CCNCC3)cc2Nc2nccc(N3CCOCC3)c21]

At 0° C., TFA (26.8 mL, 350.07 mmol, 30 eq.) in DCM (100 mL) was added dropwise to a solution of Intermediate 687 (5.9 g, 11.67 mmol) in DCM (145 mL). The reaction mixture was stirred at room temperature for 18 h. The mixture was diluted with DCM, water, and aqueous NH₄OH (30%). The mixture was stirred at room temperature for 1 h and extracted with DCM. The organic layer was dried over MgSO₄, filtered, and the solvent was evaporated. The residue was triturated in ACN, filtered, washed with ACN, and dried to give Intermediate 688 (3.47 g, yield: 73%) as a white solid.

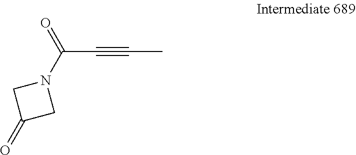

3-Azetidinone hydrochloride (CAS [17557-84-5], 65 g, 604 mmol) was added in one portion to a solution of 2-butynoic acid (CAS [590-93-2], 55.9 g, 665 mmol, 1.1 eq.) and Et₃N (253 mL, 1813 mmol, 3 eq.) in DCM (1000 mL) at 0° C. T3P (CAS [68957-94-8], 50% in EtOAc, 577 g, 907 mmol, 1.5 eq.) was then added slowly. The reaction mixture was stirred at 0° C. for 4 h. Water (800 mL) was added slowly to the mixture and the cooling bath was removed. The mixture was extracted with the DCM:MeOH (10:1, 4×1000 mL). The combined organic layers were dried (MgSO₄), filtered, and concentrated. The residue was purified by column chromatography over silica gel (eluent: petroleum ether/EtOAc 100:0 to 50:50) to afford Intermediate 689 (53.1 g, yield: 48%) as a white solid.

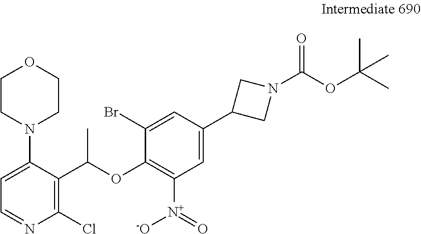

At 0° C. DIAD [2446-83-5] (1.4 mL, 1.027 g/mL, 7.11 mmol) was added to a mixture of Intermediate 571 (2 g, 5.359 mmol), Intermediate 26 (1.45 g, 5.974 mmol), PPh₃ (1.87 g, 7.129 mmol) in THF (70 mL). This reaction was stirred at room temperature for a weekend, then the reaction was poured out onto water and extracted twice with EtOAc. The organic layer was separated, dried over MgSO₄, filtered, and concentrated to dryness. Purification by silica gel chromatography (SiO₂ 15-40 μm, mobile phase: Gradient from 80% heptane, 20% EtoAc to 60% heptane, 40%) afforded Intermediate 690 (2.45 g, yield: 76%).

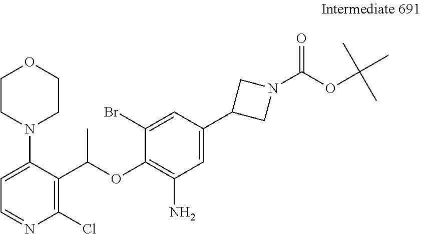 [SMILES: CC(Oc1c(N)cc(C2CN(C(=O)OC(C)(C)C)C2)cc1Br)c1c(N2CCOCC2)ccnc1Cl]

A suspension of Intermediate 690 (1.41 g, 2.358 mmol) in THF (15 mL) and iPrOH (15 mL) was hydrogenated overnight with Raney Nickel [7440-02-0] (1.5 g, 25.557 mmol) as catalyst at room temperature under 1 bar of H₂. Extra Raney Nickel [7440-02-0] (3 g, 51.113 mmol) was added and this reaction was stirred under 1 bar of H₂ for one more night. This reaction was filtered over Celite®, which was washed with DCM and iPrOH. The solvent was evaporated until dryness to give Intermediate 691 (1.42 g, quant yield).

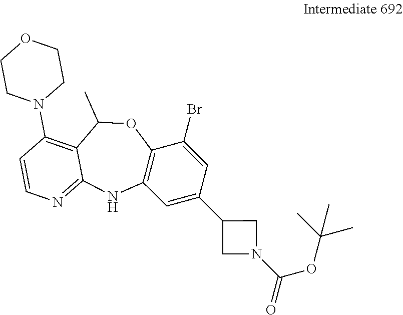 [SMILES: CC1Oc2c(Br)cc(C3CN(C(=O)OC(C)(C)C)C3)cc2Nc2nccc(N3CCOCC3)c21]

TFA (1.3 mL; 17.17 mmol) was added to a solution of Intermediate 691 (2.5 g, 4.4 mmol) in 1,4-dioxane (28 mL). The reaction mixture was stirred at 90° C. for 5 h. After the reaction mixture was cooled down to room temperature, DCM, water and a 30% aqueous solution of NH₄OH were added. The mixture was stirred at room temperature for 15 min, the organic layer was decanted, dried over MgSO₄, filtered, and the solvent was evaporated and purified by chromatography over silica gel (SiO₂, eluent: from 98% DCM, 2% MeOH, 0.2% NH₄OH to 96% DCM, 4% MeOH, 0.4% NH₄OH). to give Intermediate 692.

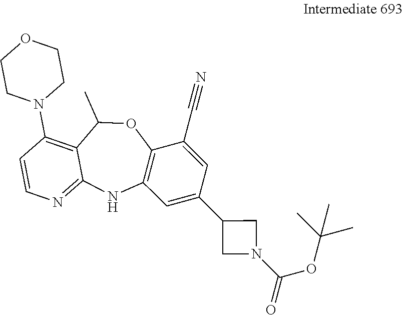 [SMILES: CC1Oc2c(C#N)cc(C3CN(C(=O)OC(C)(C)C)C3)cc2Nc2nccc(N3CCOCC3)c21]

In a sealed vessel, a solution of intermediate 692(2.04 g, 3.84 mmol), Potassium hexacyanoferrate(II) trihydrate [CAS: 14459-95-1] (811 mg, 1.92 mmol) and KOAc [CAS: 127-08-2] (188 mg, 1.92 mmol) in 1,4-dioxane (17 mL) and water (17 mL) was purged with nitrogen and XPhosPdG3 [CAS: 1445085-55-1] (325 mg, 0.38 mmol) and tBu XPhos [CAS: 564483-19-8] (163 mg, 0.38 mmol) were added. The reaction mixture was purged again with nitrogen and stirred at 100° C. for 2 h, then the mixture was poured in water and EtOAc. The organic layer was separated and washed with brine, dried over MgSO₄, filtered, and the solvent evaporated to dryness. Purification by chromatography over silica gel (SiO₂, eluent: from 100% DCM to 97% DCM, 3% MeOH, 0.3% NH₄OH) Afforded Intermediate 693 (1.75 g, yield: 93%).

### Intermediate 694A and Intermediate 694B

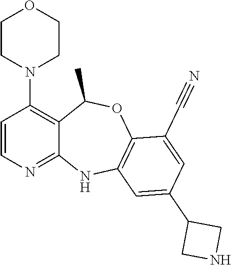 [SMILES: C[C@H]1Oc2c(C#N)cc(C3CNC3)cc2Nc2nccc(N3CCOCC3)c21]

Intermediate 694A (*R) pure stereoisomer but absolute stereochemistry undetermined.

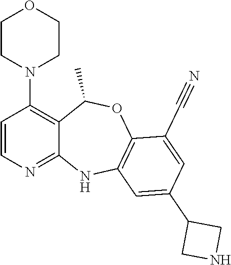 [SMILES: C[C@@H]1Oc2c(C#N)cc(C3CNC3)cc2Nc2nccc(N3CCOCC3)c21]

Intermediate 694B (*S) pure stereoisomer but absolute stereochemistry undetermined. At 0° C., TFA (8.22 mL; 107.42 mmol) in DCM (30 mL) was added dropwise to a solution of intermediate 693(1.71 g, 3.58 mmol) in DCM (40 mL). The reaction mixture was stirred at room temperature for 18 h, then diluted with DCM, water and a 30% aqueous solution of NH₄OH. The mixture was stirred at room temperature for 1 h and extracted with DCM. The organic layer was decanted, dried over MgSO₄, filtered, and the solvent was evaporated to dryness. Purification by chromatography over silica gel (irregular SiOH, Buchi®, 24 g; gradient from 97% DCM, 3% MeOH, 0.3% NH₄OH to 90% DCM, 10% MeOH, 1% NH₄OH) followed by chiral SFC separation (Chiralpak IG 5 μm 250*20 mm, mobile phase: 50% CO₂, 50% mixture of EtOH/DCM 80/20 v/v+0.9% iPrNH₂) afforded Intermediate 694A (180 mg, yield: 12%) and Intermediate 694 (254 mg, yield: 16%).

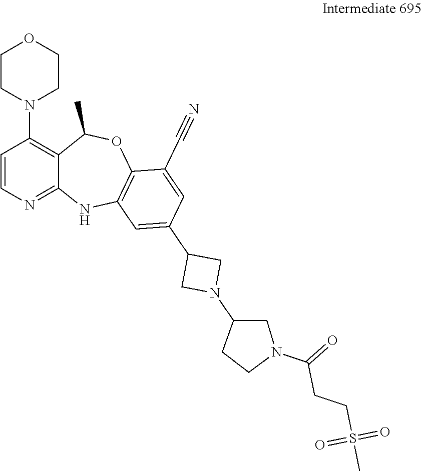 [SMILES: C[C@H]1Oc2c(C#N)cc(C3CN(C4CCN(C(=O)CCS(C)(=O)=O)C4)C3)cc2Nc2nccc(N3CCOCC3)c21]

(*R, RS) mixture of 2 stereoisomers. 1-(3-methanesulfonylpropanoyl)pyrrolidin-3-one [CAS: 2159402-64-7] (203 mg, 0.93 mmol) was added to a solution of Intermediate 694 (175 mg, 0.46 mmol) and AcOH (53 μL; 0.93 mmol) in DCE (4 mL) at room temperature. The reaction was stirred at room temperature for 30 min. NaBH(OAc)₃ [CAS: 56553-60-7] (197 mg, 0.93 mmol) was added and the reaction mixture was stirred at room temperature for 18 h. The reaction mixture was poured onto a 10% aqueous solution of K₂CO₃ and DCM. The mixture was filtered through Chromabond®, the filtrate was evaporated to dryness and purified by chromatography over silica gel (SiO₂, eluent: 96% DCM, 4% MeOH, 0.4% NH₄OH to 90% DCM, 10% MeOH, 1% NH₄OH) to afford Intermediate 695 (149 mg, 55%).

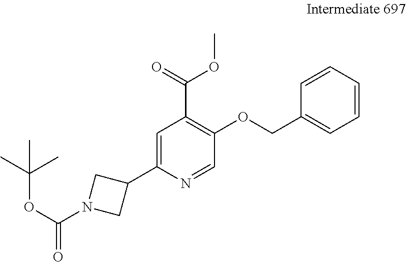 [SMILES: COC(=O)c1cc(C2CN(C(=O)OC(C)(C)C)C2)ncc1OCc1ccccc1]

[1-[(1,1-dimethylethoxy)carbonyl]-3-azetidinyl]iodozine [CAS: 206446-38-0] (197 mL, 0.5 M solution, 98.46 mmol) was added to a solution of Intermediate 100(24.4 g, 75.74 mmol), Pd(dppf)Cl₂ [CAS: 95464-05-4] (6.19 g, 7.57 mmol, 0.1 eq.), CuI [CAS: 7681-65-4] (1.44 g, 7.57 mmol, 0.1 eq.) in DMA (200 mL) under a nitrogen atmosphere at room temperature and the mixture was stirred under nitrogen at 80° C. overnight. The resulting reaction mixture was partitioned between EtOAc and saturated aqueous NaHCO₃ solution, the combined organic layers were dried (MgSO₄), filtered, and concentrated to dryness. Purification by column flash chromatography (silica; heptane/EtOAc from 100/0 to 100/0) to afford Intermediate 697 (22 g, yield: 73%).

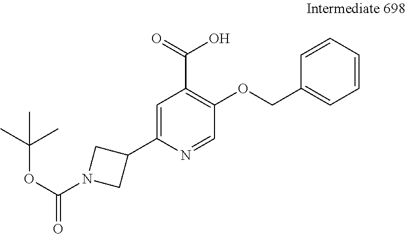 [SMILES: CC(C)(C)OC(=O)N1CC(c2cc(C(=O)O)c(OCc3ccccc3)cn2)C1]

NaOH (11.04 g, 276.1 mmol) was added to a solution of Intermediate 697 (22 g, 55.21 mmol) in MeOH (330 mL) and the mixture was heated to 60° C. 3 h. The solvent was evaporated, the mixture was cooled to room temperature and diluted with water and MTBE, the layers separated and the aqueous layer was washed with MTBE. The combined organic layer was extracted with NaOH 1 M. The combined aqueous layers were extracted with MTBE and the aqueous layer was acidified with KHSO₄ 1 M solution until acid pH and extracted with DCM. The organic layer was dried over MgSO₄, concentrated to dryness and purified by flash column chromatography (SiO₂, 120 g, MeOH/DCM gradient from 0% to 10%) to afford Intermediate 698 (16.5 g, yield: 78%).

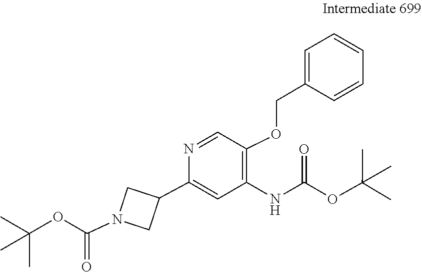 [SMILES: CC(C)(C)OC(=O)Nc1cc(C2CN(C(=O)OC(C)(C)C)C2)ncc1OCc1ccccc1]

A mixture of Intermediate 698 (16 g, 41.62 mmol) and DIPEA [CAS: 7087-68-5](17.95 mL, 125 mmol) was dissolved in t-BuOH (70 mL) and Dioxane (174 mL) under nitrogen atmosphere. DPPA [CAS: 26386-88-9] (17.94 mL, 83.24 mmol) was added and the mixture was stirred at 110° C. for 3 h. The mixture was diluted with EtOAc and washed with saturated aqueous NaHCO₃ and brine. The organic layer was dried, filtered, concentrated to dryness and purified by column flash chromatography (SiO₂, EtOAc—heptane gradient) to afford Intermediate 699 (12.9 g, yield: 68%).

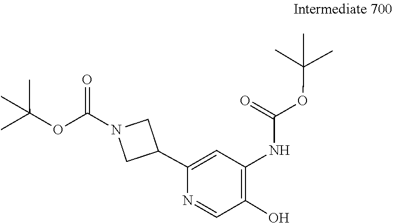 [SMILES: CC(C)(C)OC(=O)Nc1cc(C2CN(C(=O)OC(C)(C)C)C2)ncc1O]

Pd/C 10% (350 mg) was added to a solution of Intermediate 699 (12.9 g, 28.32 mmol) in MeOH (61 mL) and THF (183 mL) under Nitrogen and the mixture was purged with Nitrogen, then with H₂. The reaction mixture was stirred under an H₂ atmosphere for 15 h, then filtered through a celite pad. The filtrate was concentrated to dryness under high vacuum and purified by column flash chromatography (eluent gradient MeOH in DCM) to afford Intermediate 700 (10.2 g, yield: 98%).

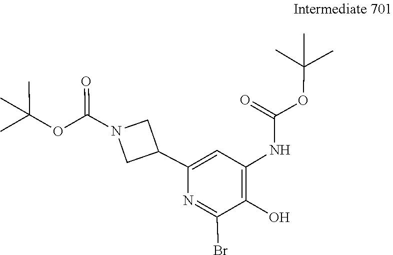 [SMILES: CC(C)(C)OC(=O)Nc1cc(C2CN(C(=O)OC(C)(C)C)C2)nc(Br)c1O]

NBS (5.36 g, 30.1 mmol) was added to a solution of Intermediate 700 (10 g, 27.37 mmol) in DMF (200 mL) at 0° C. and the resulting mixture was stirred for 2 h. The reaction mixture was added slowly to a mixture of aqueous NaHCO₃/EtOAc and KHSO₄ was added until slightly acidic pH. The organic layer was separated, washed with brine, dried over MgSO₄, filtered, and concentrated to dryness. Purification by column flash chromatography (eluent heptane-DCM/EtOAc) to afford Intermediate 701 (7.9 g, yield: 65%).

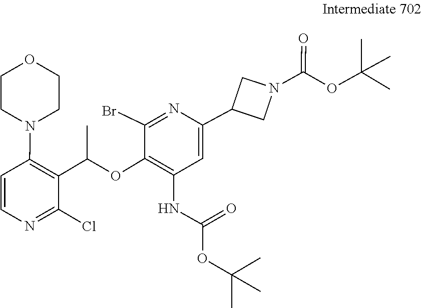 [SMILES: CC(Oc1c(NC(=O)OC(C)(C)C)cc(C2CN(C(=O)OC(C)(C)C)C2)nc1Br)c1c(N2CCOCC2)ccnc1Cl]

K₂CO₃ (4.19 g, 30.28 mmol) was added to a suspension of Intermediate 701 (3.64 g, 7.57 mmol) in DMF (30 mL). The mixture was stirred at room temperature, then Intermediate 27 (1.98 g, 7.57 mmol) was added in small portions over 5 h and the reaction mixture was stirred at room temperature overnight. The mixture was diluted with EtOAc and washed with brine. The organic layer was dried, filtered, concentrated to dryness and purified by column flash chromatography (eluent: heptane/EtOAc) to afford Intermediate 702(2.8 g, yield: 55%).

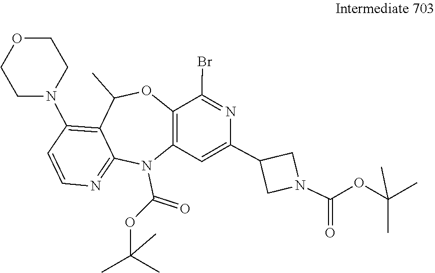 [SMILES: CC1Oc2c(cc(C3CN(C(=O)OC(C)(C)C)C3)nc2Br)N(C(=O)OC(C)(C)C)c2nccc(N3CCOCC3)c21]

A solution of Intermediate 702 (2.8 g, 4.19 mmol) and Cs₂CO₃ (2.05 g, 6.28 mmol) in Toluene (25 mL) was degassed under a nitrogen atmosphere, then S-Phos [CAS: 657408-07-6] (344 mg, 0.84 mmol) and Pd(OAc) [CAS: 3375-31-3] (188 mg, 0.84 mmol), were added. The reaction mixture was degassed again under nitrogen atmosphere and heated to 100° C. for 15 h. The reaction mixture was cooled to room temperature, concentrated to dryness and purified by column flash chromatography (eluting with heptane:DCM (9:1)/EtOAc) to afford Intermediate 703(1.45 g, yield: 55

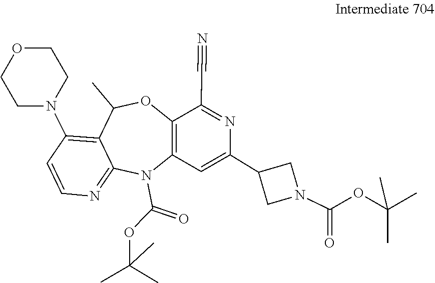 [SMILES: CC1Oc2c(cc(C3CN(C(=O)OC(C)(C)C)C3)nc2C#N)N(C(=O)OC(C)(C)C)c2nccc(N3CCOCC3)c21]

A mixture of Intermediate 703 (1.45 g, 2.29 mmol) and Zinc dust (180 mg, 2.75 mmi-ol) in DMA (24.5 mL) under a nitrogen atmosphere was stirred for 10 min, then Zinc cyanide [CAS: 557-21-1] (1.08 g, 9.17 mmol) and Pd(dppf)Cl₂ [CAS: 95464-05-4](375 mg, 0.46 mmol), was added and the mixture was heated to 110° C. for 16 h. Water was added, the mixture was extracted with EtOAc, the organic layer was separated, dried over MgSO₄, filtered, concentrated to dryness and purified by flash column chromatography on silica gel (heptane/EtOAc, gradient from 5% to 90%) to afford Intermediate 704 (814 mg, yield: 61%).

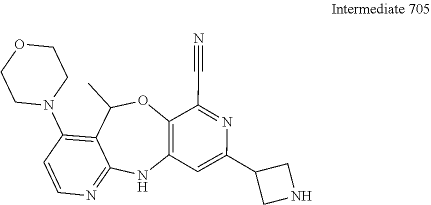 [SMILES: CC1Oc2c(cc(C3CNC3)nc2C#N)Nc2nccc(N3CCOCC3)c21]

Intermediate 704 (0.51 g, 0.88 mmol) was dissolved in a mixture of TFA (4 mL) and DCM (6 mL) and the reaction mixture was stirred for 3 h at room temperature. The mixture was evaporated to dryness, then co-evaporated with toluene to afford Intermediate 705 (879 mg, quant yield) which was used as without any further purification.

### Intermediate 706A and Intermediate 706B

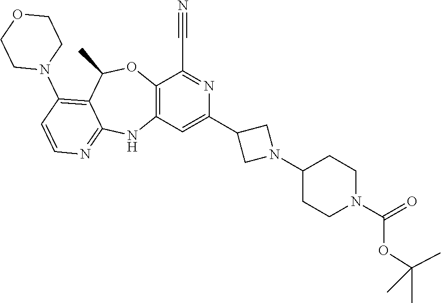 [SMILES: C[C@H]1Oc2c(cc(C3CN(C4CCN(C(=O)OC(C)(C)C)CC4)C3)nc2C#N)Nc2nccc(N3CCOCC3)c21]

Intermediate 706A (*R) pure stereoisomer but absolute stereochemistry undetermined.

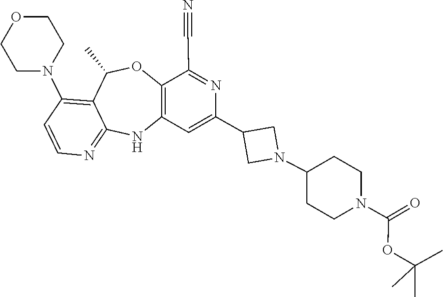 [SMILES: C[C@@H]1Oc2c(cc(C3CN(C4CCN(C(=O)OC(C)(C)C)CC4)C3)nc2C#N)Nc2nccc(N3CCOCC3)c21]

Intermediate 706B (*S) pure stereoisomer but absolute stereochemistry undetermined. tert-Butyl-4-oxopiperidine-1-carboxylate [CAS: 79099-07-3] (0.35 g, 1.76 mmol) was added to a solution of Intermediate 705 (840 mg, 0.88 mmol) and Et₃N [CAS: 121-44-8] (0.6 mL, 4.41 mmol) in DCE (12 mL) and the reaction mixture was stirred for 1 h. Sodium triacetoxyborohydride [CAS: 56553-60-7] (370 mg, 1.76 mmol) was then added and the mixture was stirred at room temperature for 18 h. Saturated aqueous NaHCO₃ was added, the mixture was extracted with DCM and the organic phase was washed with brine, separated, dried over MgSO₄, filtered, and evaporated to dryness. Purification by flash column chromatography (gradient starting with EtOAc in 5% to 100%, then MeOH-DCM (0% to 100%) followed by normal phase chiral chromatography (Phenomenex Lux Amylose-1 250×30 mm 5 μm 75% heptane+0.1% DEA−25% iPrOH:EtOH (9:1)+0.1% DEA to 100% iPrOH:EtOH (9:1)+0.1% DEA) afforded Intermediate 706A (116 mg, yield: 28%) and Intermediate 706B(125 mg, yield: 30%).

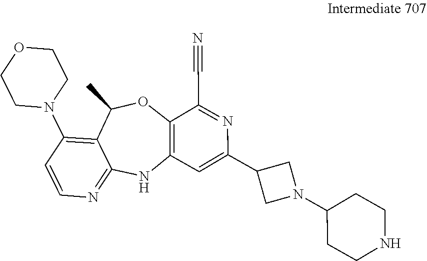 [SMILES: C[C@H]1Oc2c(cc(C3CN(C4CCNCC4)C3)nc2C#N)Nc2nccc(N3CCOCC3)c21]

Intermediate 707 (*R) pure stereoisomer but absolute stereochemistry undetermined. Intermediate 706A (107 mg, 0.19 mmol) was dissolved in DCM (1.15 mL), TFA (0.76 mL) was added and the reaction mixture was stirred for 3 h at room temperature. The mixture was evaporated to dryness to afford Intermediate 707 (88 mg, quant yield) which was used as such without further purification.

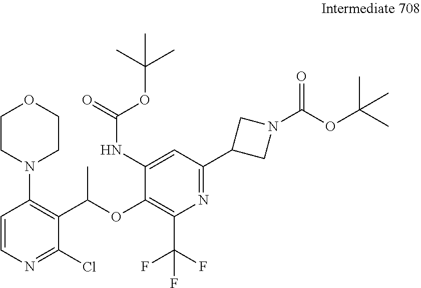 [SMILES: CC(Oc1c(NC(=O)OC(C)(C)C)cc(C2CN(C(=O)OC(C)(C)C)C2)nc1C(F)(F)F)c1c(N2CCOCC2)ccnc1Cl]

K₂CO₃ (19.51 g, 141.2 mmol) was added to a suspension of Intermediate 529 (15.3 g, 35.3 mmol) in DMF (200 mL), the mixture was stirred at room temperature, then Intermediate 27 (13.83 g, 52.95 mmol) was added in small portions over 5 h. The mixture was stirred at room temperature overnight, then diluted with EtOAc and washed with brine. The organic layer was dried, filtered, concentrated to dryness and purified by column flash chromatography (SiO₂, MeOH-DCM) to afford Intermediate 708 (17.1 g, yield: 74%)

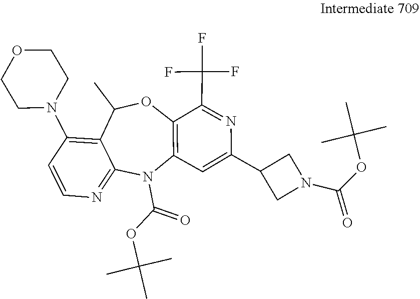 [SMILES: CC1Oc2c(cc(C3CN(C(=O)OC(C)(C)C)C3)nc2C(F)(F)F)N(C(=O)OC(C)(C)C)c2nccc(N3CCOCC3)c21]

A solution of Intermediate 708 (1.8 g, 2.74 mmol) and Cs₂CO₃ (1.34 g, 4.1 mmol) in Toluene (20 mL) was degassed under nitrogen atmosphere, then Palladium(II) acetate [CAS: 3375-31-3] (0.12 g, 0.55 mmol) and Xantphos [CAS: 161265-03-8](0.32 g, 0.55 mmol) were added. The reaction mixture was degassed again under nitrogen atmosphere and heated at 100° C. for 20 h. The mixture was then cooled to room temperature, and additional Palladium(II) acetate [CAS: 3375-31-3] and Xantphos [CAS: 161265-03-8] were added. The mixture was stirred for a further 20 h at 100° C. and then partitioned between EtOAc and brine. The combined organic layers were dried, filtered, concentrated to dryness and purified by column flash chromatography (EtOAc-heptane) to afford Intermediate 709 (934 mg, yield: 55%).

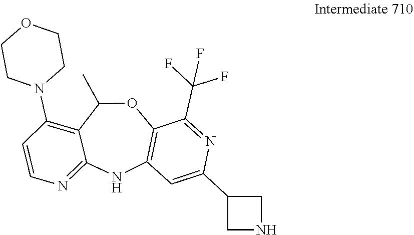 [SMILES: CC1Oc2c(cc(C3CNC3)nc2C(F)(F)F)Nc2nccc(N3CCOCC3)c21]

Intermediate 709 (2.5 g, 4.02 mmol) was dissolved in a mixture of TFA (14 mL) and DCM (21 mL) and the reaction mixture was stirred for 3 h at room temperature. The mixture was evaporated to dryness, then co-evaporated with toluene to afford Intermediate 710 (3.98 g, quant yield) which was used without any further purification.

### Intermediate 711A, Intermediate 711B, Intermediate 711C, and Intermediate 711D,

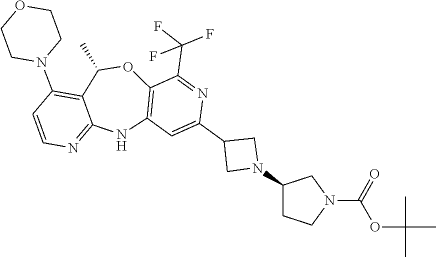 [SMILES: C[C@@H]1Oc2c(cc(C3CN([C@@H]4CCN(C(=O)OC(C)(C)C)C4)C3)nc2C(F)(F)F)Nc2nccc(N3CCOCC3)c21]

Intermediate 711A (*S, *R) pure stereoisomer but absolute stereochemistry undetermined.

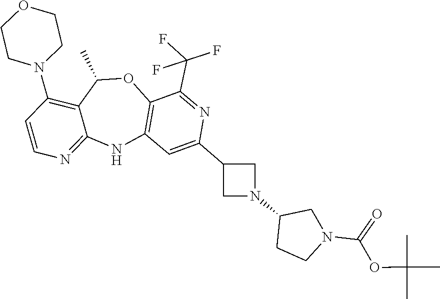 [SMILES: C[C@@H]1Oc2c(cc(C3CN([C@H]4CCN(C(=O)OC(C)(C)C)C4)C3)nc2C(F)(F)F)Nc2nccc(N3CCOCC3)c21]

Intermediate 711B (*S, *S) pure stereoisomer but absolute stereochemistry undetermined.

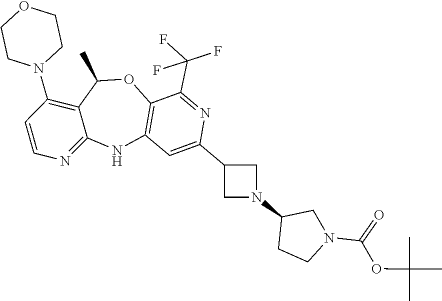 [SMILES: C[C@H]1Oc2c(cc(C3CN([C@@H]4CCN(C(=O)OC(C)(C)C)C4)C3)nc2C(F)(F)F)Nc2nccc(N3CCOCC3)c21]

Intermediate 711C (*R, *R) pure stereoisomer but absolute stereochemistry undetermined.

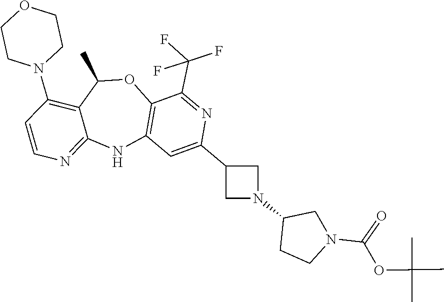 [SMILES: C[C@H]1Oc2c(cc(C3CN([C@H]4CCN(C(=O)OC(C)(C)C)C4)C3)nc2C(F)(F)F)Nc2nccc(N3CCOCC3)c21]

Intermediate 711D (*R, *S) pure stereoisomer but absolute stereochemistry undetermined.

N-Boc-3-pyrrolidinone [CAS: 101385-93-7] (1.48 g, 8 mmol) was added to a solution of Intermediate 710 (3.97 g, 4 mmol) and Triethylamine [CAS: 121-44-8] (2.78 mL, 20 mmol) in DCE (40 mL) and the mixture of reaction was stirred for 1 h at room temperature. Then, Sodium triacetoxyborohydride [CAS: 56553-60-7] (1.7 g, 8 mmol) was added and the mixture was stirred at room temperature for 18 h. Saturated aqueous NaHCO₃ was then added and the reaction mixture was extracted with DCM. The organic phase was washed with brine, then dried over MgSO₄, filtered, evaporated to dryness and purified by flash column chromatography (gradient of MeOH in DCM: 0% to 10%). Purification via chiral SFC (Stationary phase: Chiralpak IG 5 μm 250*20 mm, Mobile phase: 70% CO₂, 30% EtOH (0.3% iPrNH₂)) afforded a mixture of Intermediate 711A and Intermediate 711B (615 mg) and pureIntermediate 711C (337 mg, yield: 14%) and pureIntermediate 711D (340 mg, 0.58 mmol). A second purification on the mixed fraction via chiral SFC (Stationary phase: Chiralpak IG 5 μm 250*30 mm, Mobile phase: 65% CO₂, 35% iPrOH (0.3% iPrNH₂)) afforded pure Intermediate 711A (286 mg, yield: 12%) and pure Intermediate 711B (281 mg, yield: 12%).

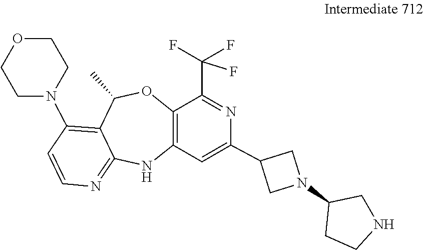 [SMILES: C[C@@H]1Oc2c(cc(C3CN([C@@H]4CCNC4)C3)nc2C(F)(F)F)Nc2nccc(N3CCOCC3)c21]

Intermediate 712 (*S, *R) pure stereoisomer but absolute stereochemistry undetermined.

Intermediate 711(248 mg, 0.42 mmol) was dissolved in DCM (4 mL) at room temperature and then TFA [CAS: 76-05-1] (2.7 mL) was added. The reaction mixture was stirred at room temperature for 3 h, and then concentrated to dryness. The residue was partitioned between EtOAc and saturated aqueous NaHCO₃. The organic layer was separated, washed with brine, dried, filtered, and concentrated to dryness to afford Intermediate 712 (206 mg, quant yield) which was used as such without any further purification.

### Intermediate 713A and Intermediate 713B

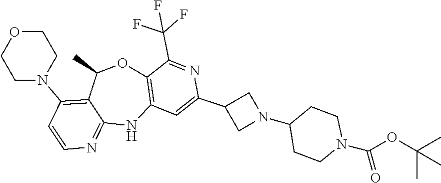 [SMILES: C[C@H]1Oc2c(cc(C3CN(C4CCN(C(=O)OC(C)(C)C)CC4)C3)nc2C(F)(F)F)Nc2nccc(N3CCOCC3)c21]

Intermediate 713A (*R) pure stereoisomer but absolute stereochemistry undetermined.

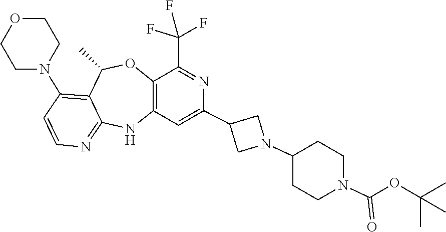 [SMILES: C[C@@H]1Oc2c(cc(C3CN(C4CCN(C(=O)OC(C)(C)C)CC4)C3)nc2C(F)(F)F)Nc2nccc(N3CCOCC3)c21]

Intermediate 713B (*S) pure stereoisomer but absolute stereochemistry undetermined. tert-Butyl-4-oxopiperidine-1-carboxylate [CAS: 79099-07-3] (1.0 g, 5 mmol) was added to a solution of intermediate 710 (2.48 g, 2.5 mmol) and Triethylamine [CAS: 121-44-8] (1.74 mL, 12.5 mmol) in DCE (20 mL) and the mixture of reaction was stirred for 1 h at room temperature. Then, Sodium triacetoxyborohydride [CAS: 56553-60-7] (1.06 g, 5 mmol) was added and the mixture was stirred at room temperature for 3 days. Saturated aqueous NaHCO₃ was then added and the reaction mixture was extracted with DCM. The organic phase was washed with brine, then dried over MgSO₄, filtered, evaporated to dryness and purified by flash column chromatography (gradient of MeOH in DCM: 0% to 10%). Purification via chiral SFC (Lux Cellulose 1; isocratic 30% MeOH) afforded Intermediate 713A (373 mg, yield: 25%) and Intermediate 713B (381 mg, yield: 25%).

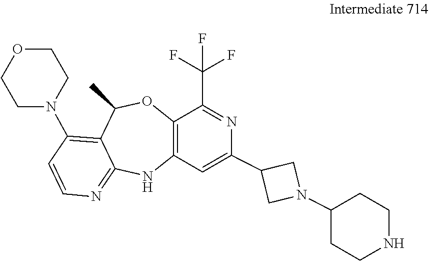 [SMILES: C[C@H]1Oc2c(cc(C3CN(C4CCNCC4)C3)nc2C(F)(F)F)Nc2nccc(N3CCOCC3)c21]

Intermediate 714 (*R) pure stereoisomer but absolute stereochemistry undetermined Intermediate 713A (370 mg, 0.61 mmol) was dissolved in a mixture of TFA (4 mL) and DCM (6 mL) and the reaction mixture was stirred for 3 h at room temperature. The mixture was evaporated to dryness, then was dried under high vacuum to afford Intermediate 714 (309 mg, quant yield) which was used without any further purification.

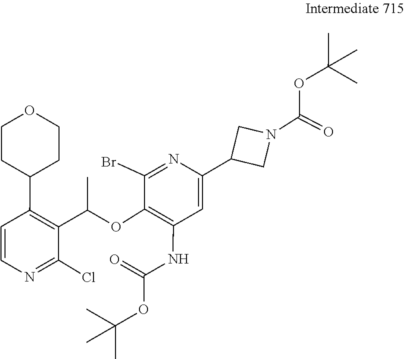 [SMILES: CC(Oc1c(NC(=O)OC(C)(C)C)cc(C2CN(C(=O)OC(C)(C)C)C2)nc1Br)c1c(C2CCOCC2)ccnc1Cl]

K₂CO₃ (4.48 g, 32.45 mmol) was added to a suspension of Intermediate 701 (3.9 g, 8.11 mmol) in DMF (40 mL). The mixture was stirred at room temperature, then Intermediate 612 (4.22 g, 16.22 mmol) was added in small portions over 3 h and the reaction mixture was stirred at 45° C. for 16 h. The mixture was partitioned between EtOAc and brine, the organic layer was dried over MgSO₄, filtered, concentrated to dryness and purified by column flash chromatography (eluent: heptane/EtOAc) to afford Intermediate 715 (2.61 g, yield: 48%).

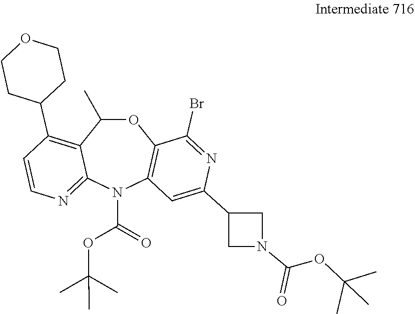 [SMILES: CC1Oc2c(cc(C3CN(C(=O)OC(C)(C)C)C3)nc2Br)N(C(=O)OC(C)(C)C)c2nccc(C3CCOCC3)c21]

Intermediate 716 was synthesized in a similar manner as Intermediate 703 using Intermediate 715 instead Intermediate 702 using 0.15 eq of S-Phos [CAS: 657408-07-6] and Pd(OAc)₂ [CAS: 3375-31-3] and purifying the Intermediate by chromatography over silica gel (eluent: heptane/EtOAc).

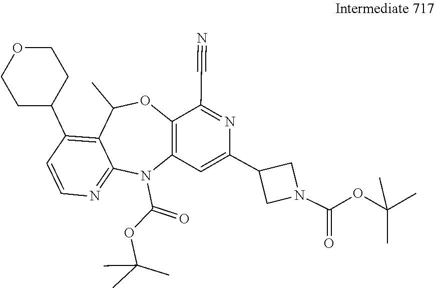 [SMILES: CC1Oc2c(cc(C3CN(C(=O)OC(C)(C)C)C3)nc2C#N)N(C(=O)OC(C)(C)C)c2nccc(C3CCOCC3)c21]

A mixture of Intermediate 716 (1.6 g, 2.53 mmol), and zinc dust [CAS: 7440-66-6] (0.2 g, 3.04 mmol) in DMA (20 mL) was stirred for 10 min under nitrogen atmosphere, then zinc cyanide [CAS: 557-21-1] (1.19 g, 10.13 mmol) and Pd(dppf)Cl₂·DCM [CAS: 95464-05-4] (0.41 g, 0.51 mmol) were added and the mixture was stirred at 110° C. for 16 h. Water and EtOAc were added and the mixture was extracted with EtOAc, the combined organic layer was separated, dried over MgSO₄, filtered, and evaporated to afford Intermediate 717 (1.16 g, yield: 73%).

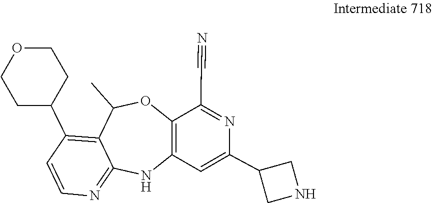 [SMILES: CC1Oc2c(cc(C3CNC3)nc2C#N)Nc2nccc(C3CCOCC3)c21]

Intermediate 717 (1.16 g, 2.01 mmol) was dissolved in a mixture of TFA (8 mL) and DCM (12 mL) and the reaction mixture was stirred for 3 h at room temperature. The mixture was evaporated to dryness, then coevaporated twice with toluene and dried under high vacuum to afford Intermediate 718 (1.75 g, quant yield) which was used without further purification.

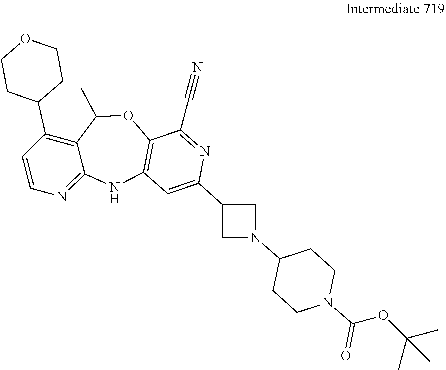 [SMILES: CC1Oc2c(cc(C3CN(C4CCN(C(=O)OC(C)(C)C)CC4)C3)nc2C#N)Nc2nccc(C3CCOCC3)c21]

tert-Butyl-4-oxopiperidine-1-carboxylate [CAS: 79099-07-3] (0.84 g, 4.2 mmol) was added to a solution of Intermediate 718 (1.75 g, 2.1 mmol) and Triethylamine [CAS: 121-44-8] (1.17 mL, 8.4 mmol) in DCE (30 mL) and the reaction mixture was stirred for 1 h at room temperature. Then, Sodium triacetoxyborohydride [CAS: 56553-60-7] (0.89 g, 4.2 mmol) was added and the mixture was stirred at room temperature for 18 h. Saturated aqueous NaHCO₃ was then added and the reaction mixture was extracted with DCM. The organic phase was washed with brine, then dried over MgSO₄, filtered, evaporated to dryness and purified by flash column chromatography (heptane-EtOAc from 5% to 100%, then MeOH in DCM: 0% to 10%) to afford Intermediate 719 (566 mg, yield: 48%).

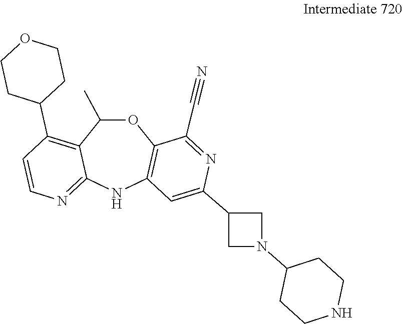 [SMILES: CC1Oc2c(cc(C3CN(C4CCNCC4)C3)nc2C#N)Nc2nccc(C3CCOCC3)c21]

TFA (4 mL) was added to a solution of Intermediate 719 (566 mg, 1.01 mmol) in DCM (6 mL) at room temperature and the reaction mixture was stirred for 3 h at room temperature. The mixture was evaporated to dryness and dried under high vacuum to afford Intermediate 720 (465 g, quant yield) which was used without further purification

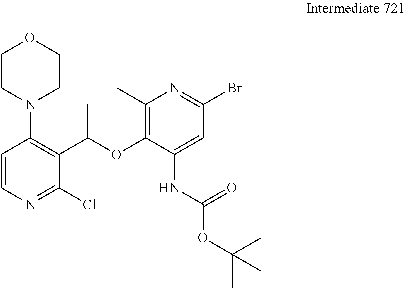 [SMILES: Cc1nc(Br)cc(NC(=O)OC(C)(C)C)c1OC(C)c1c(N2CCOCC2)ccnc1Cl]

To a solution of Intermediate 27 (3.4 g, 11.65 mmol) in DMF (50 mL) was added Intermediate 672 (2.36 g, 7.77 mmol) and K₂CO₃ (215 g, 15.54 mmol) at room temperature and the mixture was stirred overnight. More K₂CO₃ (2.15 g, 15.54 mmol) DMF (100 mL) and Intermediate 27 (4.86 g, 18.64 mmol) were added portion wise and the mixture was stirred at room temperature overnight. The reaction mixture was then diluted with water and extracted with EtOAc and DCM. The organic layers were combined, dried with MgSO₄, filtered, concentrated to dryness, and purified by flash column chromatography (silica; EtOAc in heptane 0/100 to 100/0) to afford Intermediate 721 as a colorless solid (1.56 g, yield: 60%) Intermediate 722A and Intermediate 722B

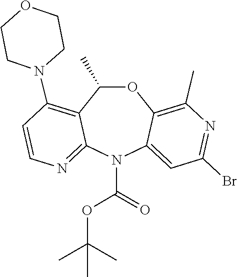 [SMILES: Cc1nc(Br)cc2c1O[C@@H](C)c1c(N3CCOCC3)ccnc1N2C(=O)OC(C)(C)C]

Intermediate 722A (*S) pure stereoisomer but absolute stereochemistry undetermined.

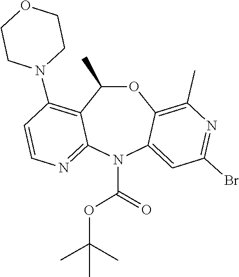 [SMILES: Cc1nc(Br)cc2c1O[C@H](C)c1c(N3CCOCC3)ccnc1N2C(=O)OC(C)(C)C]

Intermediate 722B (*R) pure stereoisomer but absolute stereochemistry undetermined To a solution of Intermediate 721 (5.38 g, 10.19 mmol) in Toluene (150 mL), were added Cs₂CO₃ (6.64 g, 20.39 mmol), S-Phos [CAS: 657408-07-6] (586 mg, 1.43 mmol) and Pd(OAc)₂ [CAS: 3375-31-3] (320 mg, 1.43 mmol). under nitrogen atmosphere the reaction mixture was heated at 120° C. overnight. The reaction mixture was cooled to room temperature, filtered through Celite (rinsed with EtOAc). The organic layer obtained was washed with water dried over MgSO₄, filtered, dried, and concentrated to dryness. Purification by column flash chromatography (eluent: Petroleum ether/EtOAc/DCM 100/0/0 to 52/24/24) followed by chiral SFC separation of enantiomers (CHIRALPAK IC 5 μm 250*30 mm, Mobile phase: 60% CO₂, 40% MeOH) afforded Intermediate 722A (yield: 22%) and Intermediate 722B (yield: 21%).

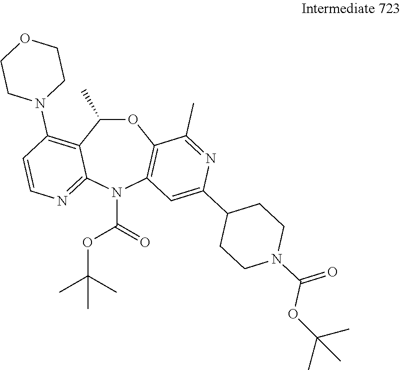 [SMILES: Cc1nc(C2CCN(C(=O)OC(C)(C)C)CC2)cc2c1O[C@@H](C)c1c(N3CCOCC3)ccnc1N2C(=O)OC(C)(C)C]

Intermediate 723 (*S) pure stereoisomer but absolute stereochemistry undetermined. Pyridine [CAS: 110-86-1] (0.33 mL, 0.982 g/mL, 4.097 mmol) and DMA (28 mL) were added to a sealed tube containing, Intermediate 722A (2 g, 4.07 mmol), 1 Boc-4-bromopiperidine [CAS: 180695-79-8] (2.15 g, 8.14 mmol), NiI₂ [CAS: 13462-90-3](0.146 g, 0.467 mmol), 4,4′-Di-tert-butyl-2,2′-dipyridyl [CAS: 72914-19-3] (0.109 g, 0.407 mmol), Zinc dust [CAS: 7440-66-6] (0.532 g, 8.136 mmol), MgCl₂ [CAS: 7786-30-3] (0.388 g, 4.07 mmol) and the mixture was purged under nitrogen atmosphere. This reaction mixture was stirred at room temperature overnight, then was poured onto water and EtOAc was added. The mixture was filtered through a pad of Celite®, the filtrate was extracted with EtOAc and the organic layer was washed with brine, decanted with Chromabond®, and the solvent was evaporated to dryness. Purification by column chromatography (Irregular SiOH 15-40 μm 120 g GraceResolv®, Gradient from 99% DCM, 1% MeOH, 0.1% NH₄OH to 94% DCM, 6% MeOH, 0.6% NH₄OH) afforded Intermediate 723 (1.3 g, yield: 54%).

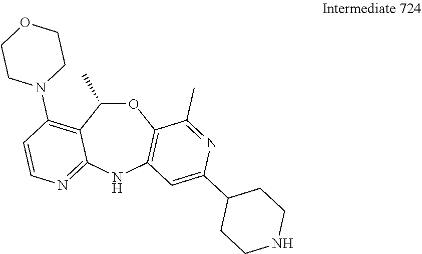 [SMILES: Cc1nc(C2CCNCC2)cc2c1O[C@@H](C)c1c(N3CCOCC3)ccnc1N2]

TFA [CAS: 76-05-1] (2.6 mL, 1.49 g/mL, 33.975 mmol) was added to a solution of Intermediate 723 (1.04 g, 1.449 mmol) in DCM (25 mL) at 0° C. and the reaction mixture was stirred overnight at room temperature. This mixture was poured onto DCM and basified with an aqueous solution of NH₄OH (30%). The organic layer was separated and washed once with brine, decanted with Chromabond® and evaporated until dryness. Purification by column chromatography (Irregular SiOH 20-45 μm 450 g GraceResolv, gradient from 100% DCM to 95% DCM, 5% MeOH, 0.5% NH₄OH) afforded Intermediate 724 (600 mg, quantitative yield).

### Preparation of Compounds

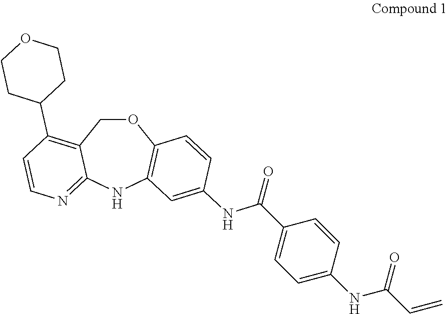 [SMILES: C=CC(=O)Nc1ccc(C(=O)Nc2ccc3c(c2)Nc2nccc(C4CCOCC4)c2CO3)cc1]

4-Acrylamidobenzoic acid ([CAS: 15286-98-3], 209 mg, 1.09 mmol, 1.3 eq.) was added to a solution of HBTU (957 mg, 2.52 mmol, 3.0 eq.) in THF (9 mL) at room temperature under inert atmosphere. Intermediate 146 (250 mg, 0.841 mmol) and DIPEA (0.439 mL; 2.52 mmol, 3.0 eq.) were added. The mixture was stirred at room temperature overnight. The reaction mixture was diluted with saturated aqueous NaHCO₃ (5 mL) and extracted with EtOAc. The organic layer was washed with saturated aqueous NH₄Cl (10 mL) and with saturated aqueous NaHCO₃ (10 mL). The combined organic layer was dried over MgSO₄, filtered, and concentrated under reduced pressure. The residue was purified by flash column chromatography (120 g SiO₂ 15-40 μm, EtOAc 100%) followed by reverse phase column chromatography (stationary phase: YMC-actus Triart-C18 10 μm 30*150 mm, mobile phase: gradient from 75% formic acid 0.1%, 25% ACN to 35% formic acid 0.1%, 65% ACN) to yield Compound 1 (125 mg, 32%).

LC MS: confirms the MW (RT: 2.48, [M+H]⁺: 471.2, Method: 1).

MP: 281.10° C. (DSC: 25° C. to 350° C./10° C. min/40 μl Al).

¹H NMR: (500 MHz, DMSO-d₆, 22° C.): d (ppm) 10.42 (s, 1H), 10.02 (s, 1H), 9.22 (br s, 1H), 8.06 (d, J=5.4 Hz, 1H), 7.92-7.97 (m, J=8.5 Hz, 2H), 7.77-7.82 (m, J=8.5 Hz, 2H), 7.59 (d, J=2.5 Hz, 1H), 6.98 (dd, J=8.5, 2.2 Hz, 1H), 6.85 (d, J=8.5 Hz, 1H), 6.73 (d, J=5.0 Hz, 1H), 6.47 (dd, J=17.0, 10.1 Hz, 1H), 6.31 (dd, J=17.0, 1.9 Hz, 1H), 5.81 (dd, J=10.1, 1.9 Hz, 1H), 5.08 (s, 2H), 3.93 (br dd, J=10.9, 3.3 Hz, 2H), 3.44-3.60 (m, 3H), 3.18 (tt, J=11.6, 3.5 Hz, 1H), 1.68 (qd, J=12.2, 3.8 Hz, 2H), 1.55-1.62 ppm (m, 2H).

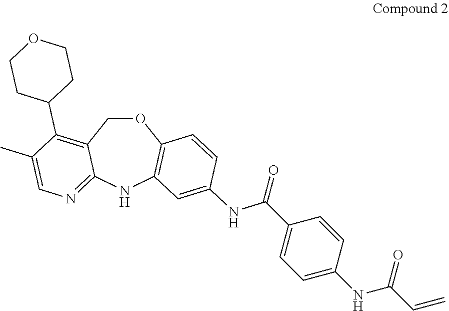 [SMILES: C=CC(=O)Nc1ccc(C(=O)Nc2ccc3c(c2)Nc2ncc(C)c(C4CCOCC4)c2CO3)cc1]

Compound 2 was synthesized in a similar manner as Compound 1 using Intermediate 153 instead of Intermediate 146.

LC MS: confirms the MW (RT: 2.58, [M+H]⁺: 485.3, Method: 1).

¹H NMR: (500 MHz, DMSO-d₆, 22° C.): δ=1s0.41 (s, 1H), 9.99 (s, 1H), 8.93 (s, 1H), 7.90-7.97 (m, 3H), 7.79 (d, J=8.8 Hz, 2H), 7.53-7.56 (m, 1H), 6.92 (d, J=8.3 Hz, 1H), 6.82 (d, J=8.5 Hz, 1H), 6.47 (dd, J=17.0, 10.1 Hz, 1H), 6.31 (dd, J=17.0, 1.9 Hz, 1H), 5.81 (dd, J=10.1, 1.9 Hz, 1H), 5.16 (s, 2H), 4.00 (br dd, J=11.2, 3.9 Hz, 2H), 3.41-3.51 (m, 2H), 3.18-3.30 (m, 1H), 2.26 (s, 3H), 1.99-2.02 (m, 1H), 1.97-2.10 (m, 4H), 1.59 ppm (br d, J=13.2 Hz, 2H)

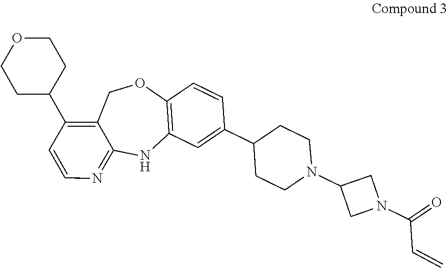 [SMILES: C=CC(=O)N1CC(N2CCC(c3ccc4c(c3)Nc3nccc(C5CCOCC5)c3CO4)CC2)C1]

Et₃N (293 μL, 2.108 mmol, 13.74 eq.) was added to a solution of Intermediate 10 (82 mg, 0.153 mmol), acrylic acid (63 μL, 0.92 mmol, 6 eq.) and EDCI·HCl (88 mg, 0.46 mmol, 3 eq.) in DMF (3 mL) and the reaction mixture was stirred at room temperature for 2 h. The reaction mixture was diluted with DCM and poured into water. The mixture was extracted twice with DCM. The organic layer was washed with brine, dried over MgSO₄, filtered, and evaporated. The residue was purified by chromatography over silica gel (SiO₂ 40 g; eluent: DCM/MeOH/NH₄OH 98/2/0.2 to 90/10/1) to give pure Compound 3 (19 mg, yield: 26%) and another impure fraction of Compound 3 (18 mg, yield: 25%).

LCMS: confirms the MW (RT: 2.57, [M+H]⁺ 475, Method: 1).

¹H NMR (DMSO-d₆, 400 MHz) d (ppm) 9.02 (s, 1H), 8.04 (d, 1H, J=5.6 Hz), 7.1-7.1 (m, 1H), 6.78 (d, 1H, J=8.1 Hz), 6.69 (d, 1H, J=5.6 Hz), 6.56 (dd, 1H, J=1.8, 8.3 Hz), 6.33 (d, 1H, J=10.1 Hz), 6.29 (d, 1H, J=10.1 Hz), 6.10 (dd, 1H, J=2.3, 16.9 Hz), 5.6-5.7 (m, 1H), 5.04 (s, 2H), 4.24 (br t, 1H, J=8.1 Hz), 4.03 (br dd, 1H, J=5.1, 8.6 Hz), 3.9-4.0 (m, 3H), 3.73 (br dd, 1H, J=5.3, 10.4 Hz), 3.51 (br t, 2H, J=10.6 Hz), 3.1-3.2 (m, 2H), 2.8-2.9 (m, 2H), 2.3-2.4 (m, 1H), 1.87 (br s, 2H), 1.5-1.8 (m, 9H)

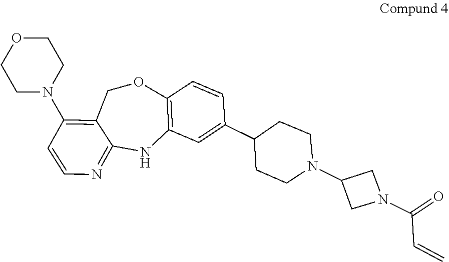 [SMILES: C=CC(=O)N1CC(N2CCC(c3ccc4c(c3)Nc3nccc(N5CCOCC5)c3CO4)CC2)C1]

Et₃N (181 μL, 1.301 mmol, 13.74 eq.) was added to a solution of Intermediate 15 (40 mg, 0.0947 mmol), acrylic acid (39 μL, 0.568 mmol, 6 eq.), and EDCI·HCl (54 mg, 0.284 mmol, 3 eq.) in DMF (1.8 mL) and the reaction mixture was stirred at room temperature for 2 h. The reaction mixture was diluted with DCM and poured into water. The mixture was extracted twice with DCM. The organic layer was washed with brine, dried over MgSO₄, filtered, and evaporated. The residue was purified by chromatography over silica gel (SiO₂, Grace, 40 g; eluent: DCM/NH₄OH/MeOH 98/2/0.2 to 90/10/1) to give Compound 4 (12 mg, yield: 27%) after freeze-drying with ACN and water.

LCMS: confirms the MW (RT: 2.56, [M+H]⁺ 476, Method: 1).

¹H NMR (DMSO-d₆, 500 MHz) d (ppm) 8.94 (s, 1H), 7.97 (d, 1H, J=5.7 Hz), 7.1-7.1 (m, 1H), 6.81 (d, 1H, J=8.2 Hz), 6.58 (dd, 1H, J=1.6, 8.2 Hz), 6.44 (d, 1H, J=5.7 Hz), 6.33 (d, 1H, J=10.4 Hz), 6.29 (d, 1H, J=10.4 Hz), 6.1-6.1 (m, 1H), 5.67 (dd, 1H, J=2.2, 10.4 Hz), 4.96 (s, 2H), 4.24 (br t, 1H, J=8.0 Hz), 4.03 (br dd, 1H, J=5.0, 8.8 Hz), 3.94 (dd, 1H, J=7.6, 10.1 Hz), 3.7-3.8 (m, 5H), 3.1-3.2 (m, 1H), 2.8-2.9 (m, 6H), 1.8-1.9 (m, 2H), 1.73 (br d, 2H, J=12.3 Hz), 1.5-1.6 (m, 2H)

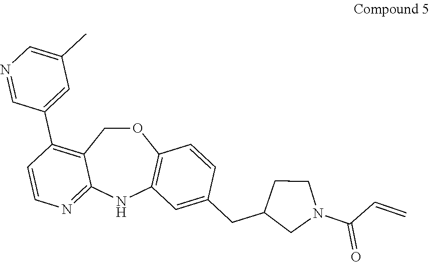 [SMILES: C=CC(=O)N1CCC(Cc2ccc3c(c2)Nc2nccc(-c4cncc(C)c4)c2CO3)C1]

Acryloyl chloride (12 μL, 0.15 mmol, 1.2 eq.) was added to Intermediate 158 (50 mg, 0.12 mmol) and Et₃N (51 μL, 0.37 mmol, 3 eq.) in DCM (0.7 mL) at 0° C. and the reaction mixture was stirred for 30 min at 0° C. The mixture was stored overnight at −20° C. Then Et₃N (25 μL, 0.19 mmol, 1.5 eq.) followed by acryloyl chloride (6 μL, 0.7 mmol, 0.6 eq.) were added to the mixture at 0° C. The reaction mixture was stirred for 1 h before it was diluted with saturated K₂CO₃ aqueous solution and extracted with DCM. The organic layer was separated, dried (Na₂SO₄), filtered, and the solvent evaporated in vacuo. The residue was purified by flash column chromatography (SiO₂, MeOH in EtOAc 0/100 to 10/90) followed by reverse phase HPLC (Stationary phase: C18 XBridge 30×100 mm 5 um, Mobile phase: Gradient from 67% 0.1% NH₄CO₃H/NH₄OH pH 9 solution in Water, 33% CH₃CN to 50% 0.1% NH₄CO₃H/NH₄OH pH 9 solution in Water, 50% CH₃CN), yielding Compound 5 (11 mg, yield: 21%) as a white solid.

LC MS: confirms the MW (RT: 1.95, [M+H]⁺: 427.21, Method: 4).

¹H NMR (400 MHz, Chloroform-d) δ (ppm) 1.56-1.76 (m, 1H) 1.97-2.11 (m, 1H) 2.37-2.46 (m, 4H) 2.47-2.70 (m, 3H) 3.15-3.23 (m, 1H) 3.42-3.55 (m, 1H) 3.60-3.78 (m, 2H) 4.92 (d, J=3.93 Hz, 2H) 5.62-5.69 (m, 1H) 6.33-6.47 (m, 2H) 6.59 (ddd, J=8.09, 3.47, 2.08 Hz, 1H) 6.63-6.69 (m, 2H) 6.87 (t, J=6.94 Hz, 1H) 7.19 (d, J=3.93 Hz, 1H) 7.49 (s, 1H) 8.15 (dd, J=5.09, 2.77 Hz, 1H) 8.41 (d, J=1.85 Hz, 1H) 8.52 (s, 1H).

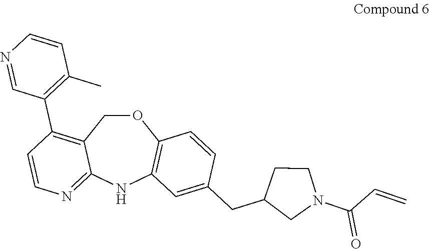 [SMILES: C=CC(=O)N1CCC(Cc2ccc3c(c2)Nc2nccc(-c4cnccc4C)c2CO3)C1]

HCl (4 M in dioxane, 1 mL, 4.0 mmol, 70.0 eq.) was added to Intermediate 159 (27 mg, 0.06 mmol) and the reaction mixture was stirred at room temperature for 15 min. The solvent was evaporated and the residue was dissolved in DCM (1 mL) and Et₃N (79 μL). To this mixture, acryloyl chloride (5.1 μL, 0.06 mmol, 1.1 eq.) was added dropwise at 0° C. The reaction mixture was stirred for 15 min at 0° C. The reaction mixture was diluted with Na₂CO₃ and extracted with DCM twice. The organic layer was separated, dried (Na₂SO₄), filtered, and the solvent evaporated in vacuo. The residue was purified by reverse phase HPLC (Stationary phase: C18 XBridge 30×100 mm 5 um, Mobile phase: Gradient from 74% 10 mM NH₄CO₃H pH 9 solution in Water, 26% CH₃CN to 58% 10 mM NH₄CO₃H pH 9 solution in Water, 42% CH₃CN) to afford Compound 6 (13 mg, yield: 51%) as a foam.

LCMS: confirms the MW (RT: 1.86, [M+H]⁺: 427.2, Method: 5).

¹H NMR (400 MHz, Chloroform-d) δ (ppm) 8.53 (dd, J=5.1, 1.4 Hz, 1H), 8.34 (s, 1 H), 8.17 (dd, J=4.9, 2.5 Hz, 1H), 7.24 (d, J=4.4 Hz, 1H), 7.19-7.23 (m, 1H), 6.81-6.88 (m, 1H), 6.66 (dd, J=4.2, 1.8 Hz, 1H), 6.54-6.61 (m, 2H), 6.32-6.48 (m, 2H), 5.62-5.70 (m, 1H), 4.65-4.74 (m, 2H), 3.58-3.79 (m, 2H), 3.40-3.55 (m, 1H), 3.13-3.23 (m, 1H), 2.32-2.72 (m, 3H), 2.20 (s, 3H), 1.98-2.12 (m, 1H), 1.66 (ddq, J=35.3, 12.4, 8.7, 8.7, 8.7 Hz, 1H).

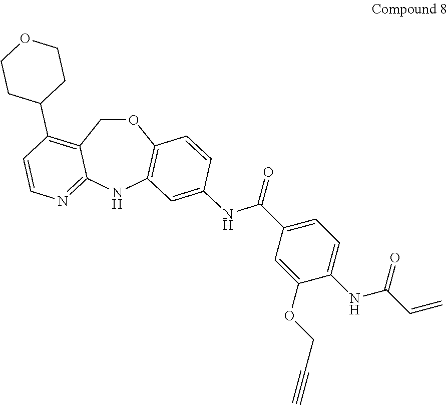 [SMILES: C#CCOc1cc(C(=O)Nc2ccc3c(c2)Nc2nccc(C4CCOCC4)c2CO3)ccc1NC(=O)C=C]

LHMDS (1 M in THF, 565 μL, 0.57 mmol, 4 eq.) was added to a solution of Intermediate 146 (42 mg, 0.14 mmol) and Intermediate 160 (50 mg, 0.16 mmol, 1.1 eq.) in THF (0.4 mL) and the reaction mixture was stirred at room temperature for 15 min. The mixture was diluted with saturated aqueous NH₄Cl and extracted with EtOAc. The organic layer was separated, washed with water, brine, dried (Na₂SO₄), filtered, and concentrated in vacuo. The residue was purified by flash column chromatography (silica; 7 N NH₃ in MeOH in DCM 0/100 to 3/97) followed by flash column chromatography (silica; EtOAc in heptane 50/50 to 100/0) to yield Compound 8 (28 mg, yield: 38%) as a yellow solid.

LCMS: confirms the MW (RT: 1.99, [M+H]⁺: 525.21, Method: 4).

¹H NMR (400 MHz, Chloroform-d) δ (ppm) 1.70 (br d, J=12.72 Hz, 2H) 1.86 (qd, J=12.48, 4.16 Hz, 2H) 2.62 (t, J=2.43 Hz, 1H) 3.00-3.18 (m, 1H) 3.58 (td, J=11.79, 1.62 Hz, 2H) 4.11 (dd, J=11.10, 3.93 Hz, 2H) 4.89 (d, J=2.31 Hz, 2H) 5.11 (s, 2H) 5.84 (dd, J=10.17, 1.16 Hz, 1H) 6.33 (dd, J=16.88, 10.17 Hz, 1H) 6.47 (dd, J=16.88, 1.16 Hz, 1H) 6.70 (d, J=5.32 Hz, 1H) 6.84 (dd, J=8.55, 2.31 Hz, 1H) 6.94 (d, J=8.55 Hz, 1H) 6.98 (s, 1H) 7.41-7.49 (m, 2H) 7.67 (d, J=1.85 Hz, 1H) 7.77 (s, 1H) 8.03 (s, 1H) 8.09 (d, J=5.32 Hz, 1H) 8.63 (d, J=8.32 Hz, 1H).

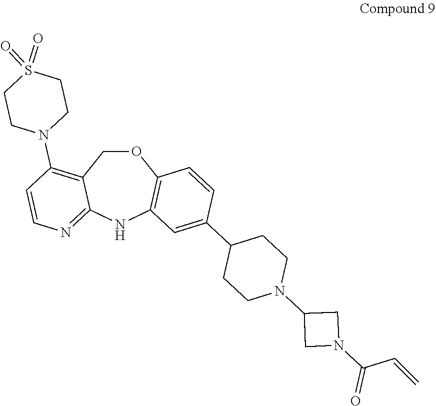 [SMILES: C=CC(=O)N1CC(N2CCC(c3ccc4c(c3)Nc3nccc(N5CCS(=O)(=O)CC5)c3CO4)CC2)C1]

Compound 9 was synthesized in a similar manner as Compound 3 using Intermediate 164 instead of Intermediate 10.

LC MS: confirms the MW (RT: 2.14, [M+H]⁺: 524.3, Method: 1).

¹H NMR (DMSO-d₆, 500 MHz) d (ppm) 8.95 (s, 1H), 7.91 (d, 1H, J=5.4 Hz), 7.02 (d, 1H, J=1.6 Hz), 6.75 (d, 1H, J=8.2 Hz), 6.53 (dd, 1H, J=1.9, 8.2 Hz), 6.47 (d, 1H, J=5.7 Hz), 6.24 (dd, 1H, J=10.4, 17.0 Hz), 6.03 (dd, 1H, J=2.2, 17.0 Hz), 5.60 (dd, 1H, J=2.2, 10.4 Hz), 4.95 (s, 2H), 4.17 (t, 1H, J=7.9 Hz), 3.97 (dd, 1H, J=5.0, 8.8 Hz), 3.88 (dd, 1H, J=7.6, 10.1 Hz), 3.67 (dd, 1H, J=5.2, 10.2 Hz), 3.0-3.1 (m, 1H), 2.8-2.9 (m, 2H), 2.3-2.3 (m, 1H), 1.8-1.9 (m, 2H), 1.6-1.7 (m, 2H), 1.5-1.6 (m, 2H)

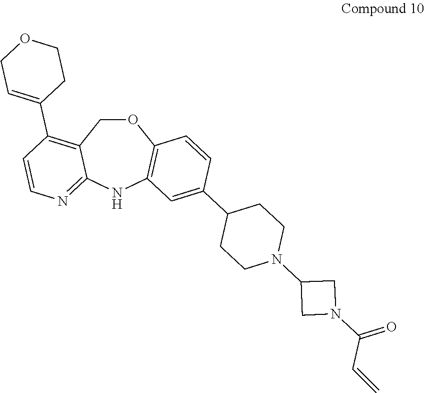 [SMILES: C=CC(=O)N1CC(N2CCC(c3ccc4c(c3)Nc3nccc(C5=CCOCC5)c3CO4)CC2)C1]

Compound 10 was synthesized in a similar manner as Compound 3 using Intermediate 168 instead of Intermediate 10.

LC MS: confirms the MW (RT: 2.62, [M+H]⁺: 473.2, Method: 1).

¹H NMR (500 MHz, DMSO-d₆, 22° C.): δ (ppm) 9.15 (s, 1H), 8.04 (d, J=5.0 Hz, 1H), 7.11 (d, J=2.2 Hz, 1H), 6.80 (d, J=7.9 Hz, 1H), 6.57-6.60 (m, 2H), 6.28-6.34 (m, 1H), 6.08-6.13 (m, 1H), 5.65-5.71 (m, 2H), 4.94 (s, 2H), 4.18-4.26 (m, 3H), 4.04 (dd, J=9.0, 5.2 Hz, 1H), 3.94 (dd, J=10.4, 7.3 Hz, 1H), 3.83 (t, J=5.4 Hz, 2H), 3.74 (dd, J=10.4, 5.0 Hz, 1H), 3.11-3.16 (m, 1H), 2.84-2.93 (m, 2H), 2.44-2.48 (m, 1H), 2.28 (br d, J=2.2 Hz, 2H), 2.08 (s, 2H), 1.83-1.95 (m, 2H), 1.74 (br d, J=13.6 Hz, 2H), 1.51-1.63 (m, 2H).

MP: 179.1° C. (DSC: 25° C. to 350° C./10° C. min/40 μl Al).

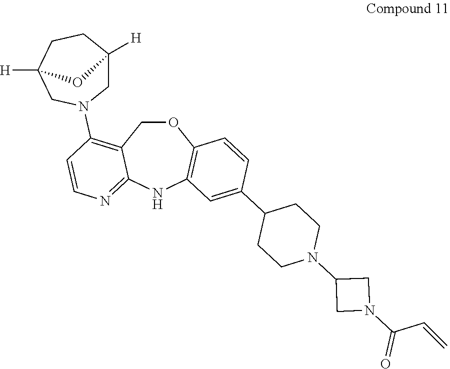 [SMILES: C=CC(=O)N1CC(N2CCC(c3ccc4c(c3)Nc3nccc(N5C[C@H]6CC[C@@H](C5)O6)c3CO4)CC2)C1]

Compound 11 was synthesized in a similar manner as Compound 3 using Intermediate 172 instead of Intermediate 10.

LC MS: confirms the MW (RT: 2.67, [M+H]⁺: 502.3, Method: 1).

¹H NMR: (500 MHz, DMSO-d₆, 22° C.): δ (ppm) 8.89 (s, 1H), 7.95 (d, J=5.7 Hz, 1H), 6.82 (d, J=8.2 Hz, 1H), 6.58 (br d, J=8.2 Hz, 1H), 6.42 (d, J=5.4 Hz, 1H), 6.31 (dd, J=17.0, 10.4 Hz, 1H), 6.10 (dd, J=17.0, 2.2 Hz, 1H), 5.64-5.69 (m, 1H), 4.98 (s, 2H), 4.36 (br s, 2H), 4.24 (br t, J=8.0 Hz, 1H), 4.03 (br dd, J=8.7, 4.9 Hz, 1H), 3.89-3.99 (m, 1H), 3.74 (br dd, J=10.6, 5.5 Hz, 1H), 3.13 (br t, J=5.4 Hz, 1H), 2.91-2.98 (m, 2H), 2.83-2.90 (m, 3H), 2.36 (br d, J=1.9 Hz, 1H), 2.00 (br d, J=7.3 Hz, 2H), 1.88 (br d, J=5.4 Hz, 4H), 1.73 (br d, J=13.2 Hz, 2H), 1.48-1.66 ppm (m, 2H).

MP: 165.8° C. (DSC: 25° C. to 350° C./10° C. min/40 μl Al).

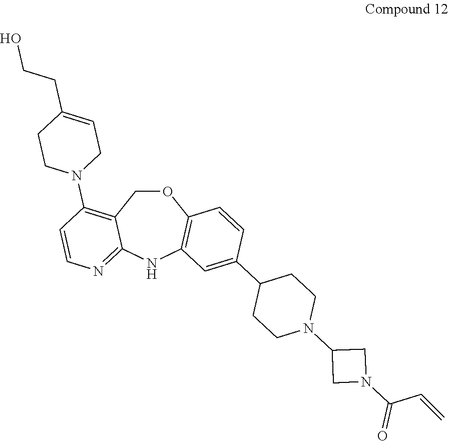 [SMILES: C=CC(=O)N1CC(N2CCC(c3ccc4c(c3)Nc3nccc(N5CC=C(CCO)CC5)c3CO4)CC2)C1]

Compound 12 was synthesized in a similar manner as Compound 3 using Intermediate 174 instead of Intermediate 10.

LC MS: confirms the MW (RT: 2.46, [M+H]⁺: 516.4, Method: 1).

¹H NMR: (500 MHz, DMSO-d₆, 22° C.): δ (ppm) 8.77 (s, 1H), 7.84 (d, J=5.4 Hz, 1H), 7.01 (s, 1H), 6.74 (d, J=7.9 Hz, 1H), 6.50 (br d, J=7.9 Hz, 1H), 6.35 (d, J=5.7 Hz, 1H), 6.24 (dd, J=17.0, 10.4 Hz, 1H), 6.03 (dd, J=16.9, 2.0 Hz, 1H), 5.60 (dd, J=10.4, 1.9 Hz, 1H), 5.44 (br s, 1H), 4.84 (s, 2H), 4.41 (br t, J=5.0 Hz, 1H), 4.17 (br t, J=7.7 Hz, 1H), 3.97 (br dd, J=8.8, 5.0 Hz, 1H), 3.80-3.92 (m, 1H), 3.67 (br dd, J=10.2, 4.9 Hz, 1H), 3.44-3.50 (m, 2H), 3.41 (br s, 2H), 3.04-3.11 (m, 1H), 3.02 (br t, J=5.4 Hz, 2H), 2.75-2.88 (m, 2H), 2.30 (br t, J=12.3 Hz, 1H), 2.07-2.19 (m, 4H), 1.73-1.90 (m, 2H), 1.66 (br d, J=12.3 Hz, 2H), 1.44-1.59 ppm (m, 2H).

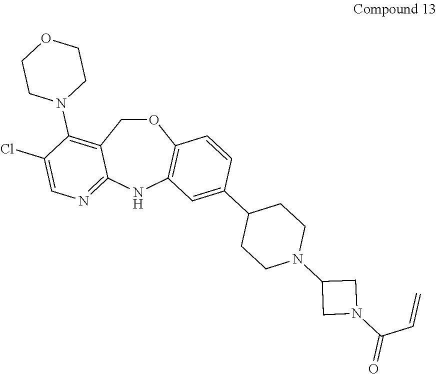 [SMILES: C=CC(=O)N1CC(N2CCC(c3ccc4c(c3)Nc3ncc(Cl)c(N5CCOCC5)c3CO4)CC2)C1]

Compound 13 was synthesized in a similar manner as Compound 3 using Intermediate 178 instead of Intermediate 10.

LC MS: confirms the MW (RT: 2.87, [M+H]⁺: 510.3, Method: 1).

¹H NMR: (500 MHz, DMSO-d₆, 22° C.): δ (pm) 9.22 (br s, 1H), 8.02 (s, 1H), 7.09 (br s, 1H), 6.84 (br d, J=7.9 Hz, 1H), 6.63 (br d, J=7.9 Hz, 1H), 6.31 (br dd, J=17.0, 10.4 Hz, 1H), 6.11 (br d, J=17.0 Hz, 1H), 5.67 (br d, J=10.4 Hz, 1H), 5.10 (br s, 2H), 4.24 (br t, J=7.7 Hz, 1H), 4.04 (br s, 1H), 3.95 (br t, J=8.4 Hz, 1H), 3.34 (s, 4H), 3.09-3.17 (m, 2H), 2.82-3.03 (m, 2H), 2.28-2.45 (m, 1H), 2.08 (s, 1H), 1.89 (br d, J=4.1 Hz, 2H), 1.73 (br d, J=11.7 Hz, 2H), 1.51-1.66 ppm (m, 2H) MP: 212.7° C. (DSC: 25° C. to 300° C./10° C. min/40 μl Al).

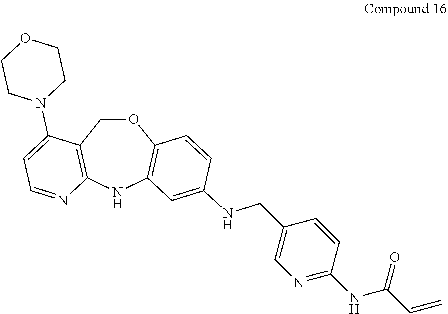 [SMILES: C=CC(=O)Nc1ccc(CNc2ccc3c(c2)Nc2nccc(N4CCOCC4)c2CO3)cn1]

Lithium hydroxide monohydrate (25 mg, 0.6 mmol, 5.0 eq.) was added to a solution of Intermediate 184 (65 mg, 0.12 mmol) in THF (5 mL) and water (0.5 mL) and the reaction mixture was stirred at room temperature for 3 h. The reaction mixture was diluted with EtOAc and washed with brine. The organic layer was dried over MgSO₄ and concentrated under vacuum. The residue was purified by flash column chromatography (SiO₂, MeOH-DCM gradient) to yield Compound 16 (31 mg, yield: 58%).

LC MS: confirms the MW (RT: 1.93, [M+H]⁺: 459.2, Method: 2).

¹H NMR: (300 MHz, Chloroform-d) δ (ppm) 2.99 (t, J=4.5 Hz, 4H), 3.71-3.94 (m, 41H), 4.14 (s, 21H), 5.01 (s, 21H), 5.76 (dd, J=10.0, 1.6 Hz, 1H), 6.08-6.16 (m, 2H), 6.25-6.53 (m, 4H), 6.80 (d, J=8.3 Hz, 1H), 7.72 (dd, J=8.6, 2.2 Hz, 1H), 7.97 (d, J=5.7 Hz, 1H), 8.26-8.44 (m, 3H), 10.06 (s, 1H).

MP: 139.7° C. (Mettler Toledo MP50), uncorrected.

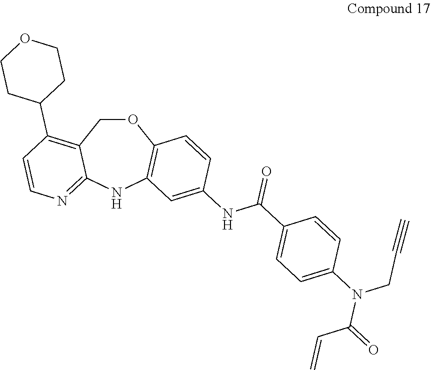 [SMILES: C#CCN(C(=O)C=C)c1ccc(C(=O)Nc2ccc3c(c2)Nc2nccc(C4CCOCC4)c2CO3)cc1]

Compound 17 was synthesized in a similar manner as Compound 1 using Intermediate 186 instead of 4-acrylamidobenzoic acid.

LC MS: confirms the MW (RT: 1.94, [M+H]⁺: 525.22, Method: 4).

¹H NMR: (400 MHz, DMSO-d₆) δ (ppm) 1.53-1.75 (m, 4H) 3.11-3.24 (m, 2H) 3.46-3.60 (m, 2H) 3.93 (dd, J=10.63, 3.47 Hz, 2H) 4.60 (d, J=2.31 Hz, 2H) 5.08 (s, 2H) 5.68 (dd, J=10.17, 2.08 Hz, 1H) 5.98-6.13 (m, 1H) 6.24 (dd, J=16.65, 2.08 Hz, 1H) 6.72 (d, J=5.32 Hz, 1H) 6.86 (d, J=8.55 Hz, 1H) 7.00 (dd, J=8.67, 2.43 Hz, 1H) 7.45 (d, J=8.55 Hz, 2H) 7.60 (d, J=2.31 Hz, 1H) 7.99-8.04 (m, 2H) 8.06 (d, J=5.09 Hz, 1 H) 9.22_(s, 1H) 10.22 (s, 1H).

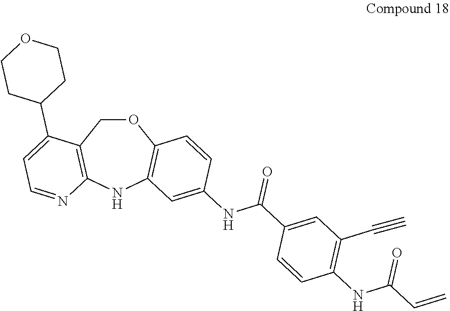 [SMILES: C#Cc1cc(C(=O)Nc2ccc3c(c2)Nc2nccc(C4CCOCC4)c2CO3)ccc1NC(=O)C=C]

KF (204 mg, 3.5 mmol, 3.0 eq.) was added to a solution of Intermediate 189 (850 mg, 1.17 mmol, 1.0 eq.) in DMF (10 mL) and the reaction mixture was stirred at 20° C. for 16 h. The reaction mixture was filtered and the filtrate was purified by preparative high-performance liquid chromatography (Column: Phenomenex Genimi NX C18 150*40 mm*5 um; isocratic water (0.225% FA)/ACN 68/32) to give Compound 18 (58 mg, yield: 9%) as a white solid.

LC MS: confirms the MW (RT: 1.97, [M+H]⁺: 495.3, Method: 4).

¹H NMR (400 MHz, DMSO-d₆) δ (ppm) 10.12 (s, 1H), 9.72 (s, 1H), 9.22 (s, 1H), 8.13-8.06 (m, 1H), 8.05-7.96 (m, 2H), 7.96-7.86 (m, 1H), 7.56 (br d, J=2.2 Hz, 1H), 7.00-6.90 (m, 1H), 6.86-6.78 (m, 1H), 6.73-6.67 (m, 1H), 6.67-6.57 (m, 1H), 6.33-6.23 (m, 1H), 5.82-5.74 (m, 1H), 5.12-4.96 (m, 2H), 4.62 (s, 1H), 3.94-3.83 (m, 2H), 3.49 (br s, 2H), 3.18-3.13 (m, 1H), 1.62 (dt, J=3.7, 12.0 Hz, 2H), 1.58-1.49 (m, 2H).

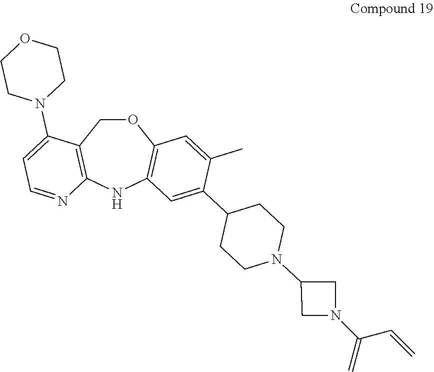 [SMILES: C=CC(=C)N1CC(N2CCC(c3cc4c(cc3C)OCc3c(N5CCOCC5)ccnc3N4)CC2)C1]

Triethylamine (54 μL, 0.39 mmol, 3.0 eq.) was added to a solution of Intermediate 197 (56 mg, 0.13 mmol) in DCM (4 mL). The mixture was cooled in an ice bath and acryloyl chloride (9 μL, 0.12 mmol, 0.9 eq.) was added dropwise. The reaction mixture was stirred at room temperature for 3 h. The reaction mixture was diluted with saturated aqueous NaHCO₃ and extracted with DCM. The organic layer was dried over MgSO₄, filtered, and concentrated under vacuum. The residue was purified by flash column chromatography (EtOAc-heptane gradient) followed by flash column chromatography (SiO₂, MeOH-DCM gradient) to yield Compound 19 (9 mg, yield: 14%).

LC MS: confirms the MW (RT: 1.54, [M+H]⁺: 490.3, Method: 2).

¹H NMR (300 MHz, Chloroform-d) δ (ppm) 1.61-1.88 (m, 4H), 1.90-2.06 (m, 2H), 2.20 (s, 3H), 2.62 (ddt, J=11.5, 7.5, 3.8 Hz, 1H), 2.84-2.99 (m, 2H), 3.02 (t, J=4.6 Hz, 4H), 3.15-3.28 (m, 1H), 3.86 (t, J=4.6 Hz, 4H), 3.98 (dd, J=10.5, 5.5 Hz, 1H), 4.05-4.18 (m, 2H), 4.24 (t, J=7.9 Hz, 1H), 5.03 (s, 2H), 5.66 (dd, J=10.1, 2.1 Hz, 1H), 6.08-6.27 (m, 1H), 6.28-6.40 (m, 2H), 6.62 (s, 1H), 6.73 (s, 2H), 7.97 (d, J=5.6 Hz, 1H).

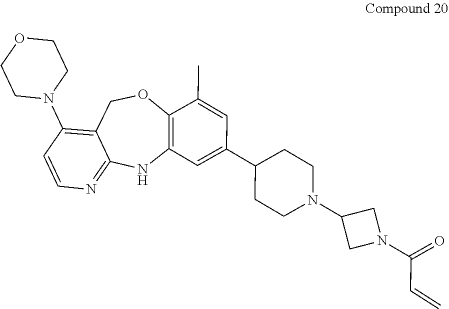 [SMILES: C=CC(=O)N1CC(N2CCC(c3cc(C)c4c(c3)Nc3nccc(N5CCOCC5)c3CO4)CC2)C1]

To a solution of Intermediate 25 (310 mg, 0.711 mmol) in DCM (10 mL), Et₃N (1.49 mL, 10.665 mmol, 15 eq.) was added. The mixture was cooled in a ice bath. Then acryloyl chloride (35 μL, 0.427 mmol, 0.6 eq.) in DCM (5 mL) was added dropwise. The reaction mixture was stirred at room temperature for 2 h. Additional acryloyl chloride (35 μL, 0.427 mmol, 0.6 eq.) was added and mixture was stirred for 3 h at room temperature. The reaction was quenched by addition of saturated aqueous NaHCO₃ and the mixture was extracted with DCM. The organic layer was dried over MgSO₄ and concentrated under vacuum. The residue was purified by column flash chromatography (DCM/DCM:MeOH (9:1) gradient) to give Compound 20 (48 mg, yield: 14%).

LCMS: confirms the MW (RT: 1.52, [M+H]⁺ 490, Method: 2).

¹H NMR (300 MHz, Chloroform-d) d (ppm) 1.65-1.91 (m, 4H), 1.90-2.05 (m, 2H), 2.25 (s, 3H), 2.36-2.52 (m, 1H1), 2.85-3.03 (m, 2H), 3.03-3.15 (m, 4H), 3.14-329 (m, 1H), 3.80-3.92 (m, 4H), 3.93-4.05 (m, 1H), 4.05-4.19 (m, 2H), 4.18-4.32 (m, 1H), 5.01 (s, 2H), 5.61-5.72 (m, 1H), 6.22 (d, J=10.1 Hz, 1), 6.30 (d, J=2.1 Hz, 1H), 6.33-6.43 (m, 1H), 6.68 (d, J=18.0 Hz, 2H), 7.84 (d, J=6.2 Hz, 1H), 8.49 (d, J=106.4 Hz, 1H).

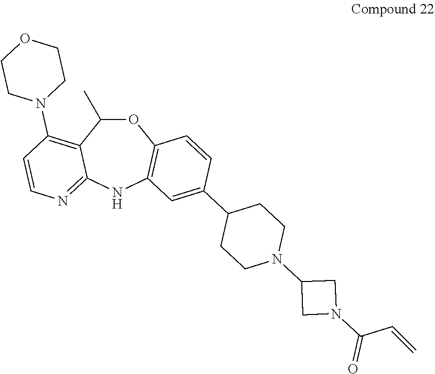 [SMILES: C=CC(=O)N1CC(N2CCC(c3ccc4c(c3)Nc3nccc(N5CCOCC5)c3C(C)O4)CC2)C1]

Compound 22 was synthesized in a similar manner as Compound 83, using Intermediate 207 instead of Intermediate 38.

LC MS: confirms the MW (RT: 1.5, [M+H]⁺: 490.2, Method: 2).

¹H NMR (300 MHz, Chloroform-d) δ (ppm) 1.40 (d, J=6.9 Hz, 3H), 1.62-2.05 (m, 6H), 2.38-2.52 (m, 1H), 2.82-3.01 (m, 4H), 3.02-3.14 (m, 2H), 3.13-3.27 (m, 1H), 3.79-3.92 (m, 4H), 3.97 (dd, J=10.5, 5.4 Hz, 1H), 4.06-4.18 (m, 2H), 4.24 (t, J=7.9 Hz, 1H), 5.66 (dd, J=10.1, 2.1 Hz, 1H), 5.83 (q, J=6.9 Hz, 1H), 6.19 (dd, J=17.0, 10.1 Hz, 1H), 6.33 (dd, J=17.0, 2.1 Hz, 1H), 6.45 (d, J=5.6 Hz, 1H), 6.60 (d, J=2.0 Hz, 1H), 6.66 (dd, J=8.3, 1.9 Hz, 1H), 6.87 (d, J=8.2 Hz, 1H), 7.06 (s, 1H), 8.00 (d, J=5.5 Hz, 1H).

MP: 224.9° C. (Mettler Toledo MP50), uncorrected.

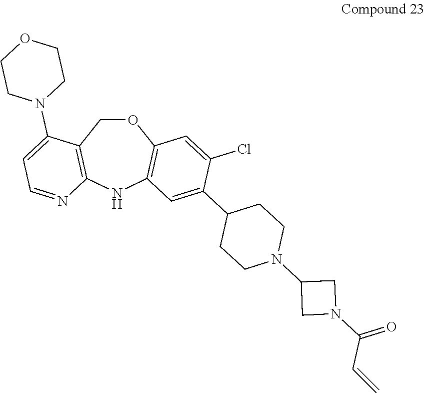 [SMILES: C=CC(=O)N1CC(N2CCC(c3cc4c(cc3Cl)OCc3c(N5CCOCC5)ccnc3N4)CC2)C1]

Triethylamine (77 μL, 0.56 mmol, 5.0 eq.) was added to a solution of Intermediate 212 (76 mg, 0.11 mmol) in DCM (4 mL), The reaction mixture was cooled in an ice bath, acryloyl chloride (7 μL, 0.089 mmol, 0.8 eq.) was added dropwise and the reaction mixture was stirred at room temperature for 3 h. The reaction mixture was quenched by addition of saturated aqueous NaHCO₃ and extracted with DCM. The combined organic layer was dried over MgSO₄ and concentrated under vacuum. The residue was purified by flash column chromatography (EtOAc-heptane gradient) followed by flash column chromatography (SiO₂, MeOH-DCM gradient) to yield Compound 23 (28 mg, yield: 49%).

LC MS: confirms the MW (RT: 1.73, [M+H]⁺: 510.2, Method: 2).

¹H NMR (300 MHz, Chloroform-d) δ (ppm) 1.56-1.76 (m, 2H), 1.88 (d, J=12.8 Hz, 2H), 1.96-2.07 (m, 2H), 2.83-2.98 (m, 3H), 3.02 (t, J=4.5 Hz, 4H), 3.14-3.31 (m, 1H), 3.87 (t, J=4.5 Hz, 41H), 3.93-4.03 (m, 1H), 4.06-4.19 (m, 2H), 4.24 (t, J=7.9 Hz, 1H), 5.03 (s, 2H), 5.66 (dd, J=10.1, 2.1 Hz, 1H), 6.12-6.27 (m, 1H), 6.28-6.45 (m, 2H), 6.65 (s, 1H), 6.82 (s, 1H), 6.95 (s, 1H), 8.00 (d, J=5.6 Hz, 1H).

MP: 250.1° C. (Mettler Toledo MP50), uncorrected.

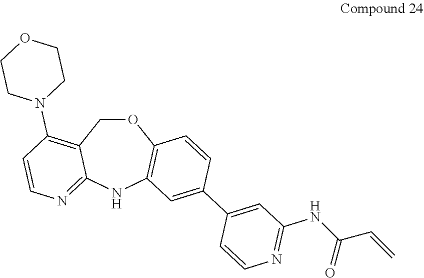 [SMILES: C=CC(=O)Nc1cc(-c2ccc3c(c2)Nc2nccc(N4CCOCC4)c2CO3)ccn1]

TFA (480 μL, 6.269 mmol, 40 eq.) was added to a solution of Intermediate 214 (83 mg, 0.157 mmol) in DCM (2.4 mL) at 0° C. and the reaction mixture was then stirred at room temperature for 5 h. The volatiles were evaporated. The residue was taken up with saturated aqueous Na₂CO₃ and DCM. The organic layer was dried with MgSO₄, filtered, and concentrated to give Compound 24 (52 mg, yield: 77%) as a yellow solid.

LC MS: confirms the MW (RT: 2.099, [M+H]⁺: 430.2, Method: 2).

¹H NMR (300 MHz, Chloroform-d) δ (ppm) 2.83-3.25 (m, 4H), 3.74-4.02 (m, 4H), 5.11 (s, 2H), 5.71-5.93 (m, 1H), 6.23-6.36 (m, 1H), 6.41 (d, J=5.6 Hz, 1H), 6.44-6.55 (m, 1H), 6.96-7.08 (m, 1H), 7.08-7.16 (m, 3H), 7.17-7.24 (m, 1H), 8.03 (d, J=5.6 Hz, 1H), 8.30 (d, J=5.3 Hz, 1H), 8.37 (s, 1H), 8.55 (s, 1H).

MP: 246.7° C. (Mettler Toledo MP50), uncorrected.

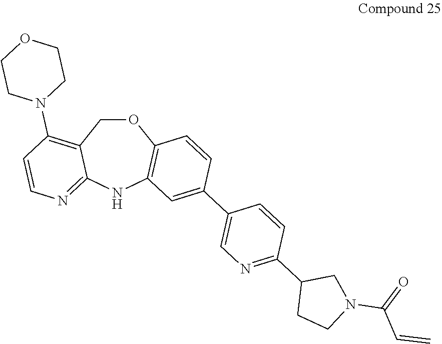 [SMILES: C=CC(=O)N1CCC(c2ccc(-c3ccc4c(c3)Nc3nccc(N5CCOCC5)c3CO4)cn2)C1]

Triethylamine (60 μL, 0.43 mmol, 1.0 eq.) was added to a solution of Intermediate 218 (185 mg, 0.43 mmol) in DCM (4 mL). The reaction mixture was cooled in an ice bath, acryloyl chloride (31 μL, 0.39 mmol, 0.9 eq.) was added dropwise and the reaction mixture was stirred at room temperature for 1 h. The reaction mixture was quenched by addition of saturated aqueous NaHCO₃ and extracted with DCM. The organic layer was dried over MgSO₄ and concentrated under vacuum. The residue was purified by reverse phase column chromatography (Phenomenex Gemini C18 30×100 mm 5 μm; gradient from 95% [0.1% HCOOH]−5% ACN to 63% [0.1% HCOOH]−37% ACN) to afford Compound 25 (29 mg, yield: 14%).

LC MS: confirms the MW (RT: 2.097, [M+H]⁺: 484.1, Method: 2).

¹H NMR (300 MHz, Chloroform-d) δ (ppm) 2.52-2.70 (m. 1H), 3.29 (t, J=4.4 Hz, 4H), 3.71 (q, J=7.0 Hz, 1H), 3.78-4.03 (m, 2H), 4.12 (t, J=4.4 Hz, 6H), 4.23-4.43 (m, 1H), 5.35 (s, 21), 5.88-5.97 (m, 1H), 6.55-6.67 (m, 2H), 6.67-6.80 (m, 1H), 7.20 (dd, J=8.3, 2.0 Hz, 1H), 7.25-7.31 (m, 2H), 7.43-7.53 (m, 1H), 7.76 (q, J=13.1, 12.4 Hz, 1H), 7.93-8.03 (m, 1H), 8.27 (d, J=5.6 Hz, 1H), 8.94 (t, J=2.2 Hz, 1H).

MP: 154.7° C. (Mettler Toledo MP50), uncorrected.

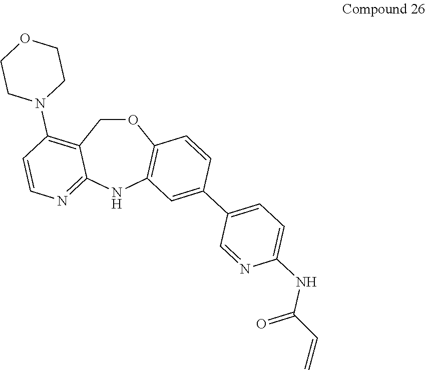 [SMILES: C=CC(=O)Nc1ccc(-c2ccc3c(c2)Nc2nccc(N4CCOCC4)c2CO3)cn1]

Compound 26 was synthesized in a similar manner as Compound 24 using Intermediate 220 instead of Intermediate 214.

LC MS: confirms the MW (RT: 2.182, [M+H]⁺: 430.2, Method: 2).

¹H NMR (300 MHz, Chloroform-d) δ (ppm) 3.07 (t, 4H), 3.90 (t, 4H), 5.12 (s, 2H), 5.72-5.83 (m, 1H), 6.27-6.56 (m, 3H), 6.87-6.96 (m, 1H), 7.05 (d, 1H), 7.16-7.24 (m, 1H), 7.82-7.92 (m, 1H), 8.02 (d, J=5.7 Hz, 1H), 8.43 (d, J=8.7 Hz, 1H), 8.59-8.68 (m, 1H), 9.01 (s, 1H), 10.44 (s, 1H).

MP: 246.7° C. (Mettler Toledo MP50), uncorrected.

### Compound 27 and Compound 48

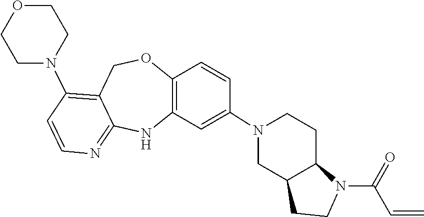 [SMILES: C=CC(=O)N1CC[C@H]2CN(c3ccc4c(c3)Nc3nccc(N5CCOCC5)c3CO4)CC[C@H]21] [1170] Compound 27: Pair of enantiomers, relative stereochemistry CIS

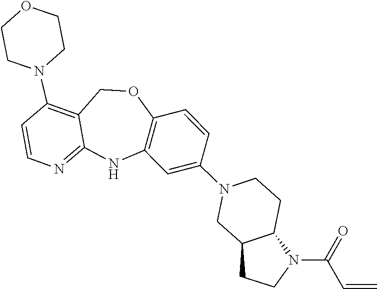 [SMILES: C=CC(=O)N1CC[C@H]2CN(c3ccc4c(c3)Nc3nccc(N5CCOCC5)c3CO4)CC[C@@H]21]

Compound 48: Pair of enantiomers, relative stereochemistry TRANS Triethylamine (470 μL, 3.4 mmol, 5.0 eq.) was added to a solution of Intermediate 222 (429 mg, 0.68 mmol) in DCM (4 mL) and the reaction mixture was cooled in an ice bath. Acryloyl chloride (60 μL, 0.74 mmol, 1.1 eq.) was added dropwise and the reaction mixture was stirred at room temperature for 3 h. The reaction mixture was quenched by addition of saturated aqueous NaHCO₃ and extracted with DCM. The organic layer was dried over MgSO₄, filtered, and concentrated under vacuum. The residue was purified by flash column chromatography on silica gel (EtOAc-heptane gradient) followed by flash column chromatography (SiO₂, MeOH-DCM gradient), and by reverse phase chromatography (Phenomenex Gemini C18 100A column (100 mm×30 mm I.D.; 5 μm particles); gradient from 70% NH₄CO₃ aqueous solution (25 mM+ACN 10%)/30% (ACN/MeOH 1/1) to 27% NH₄CO₃ aqueous solution (25 mM+ACN 10%)/73% (ACN/MeOH 1/1)) to afford Compound 27 (97 mg, yield: 31%) and Compound 48 (5 mg, yield: 2%)

### Compound 27

LC MS: confirms the MW (RT: 2.071, [M+H]⁺: 462.2, Method: 2).

¹H NMR (300 MHz, Chloroform-d) δ (ppm) 1.57-1.84 (m, 1H), 1.86-2.01 (m, 1H), 2.19-2.37 (m, 2H), 2.38-2.54 (m, 1H), 2.55-2.78 (m, 1H), 2.92-3.00 (m, 4H), 3.05 (dd, J=12.8, 3.6 Hz, 1H), 3.29-3.76 (m, 4H), 3.81-3.90 (m, 4H), 3.88-4.32 (m, 1H), 5.01 (s, 2H), 5.67 (dd, J=8.5, 3.8 Hz, 1H), 6.30-6.47 (m, 5H), 6.84 (d, J=7.9 Hz, 1H), 7.22-7.37 (m, 1H), 8.00 (d, J=5.6 Hz, 1H).

MP: 138.0° C. (Mettler Toledo MP50), uncorrected.

### Compound 48

LC MS: confirms the MW (RT: 1.935, [M+H]⁺: 462.2, Method: 2).

¹H NMR (300 MHz, Chloroform-d) δ (ppm) 1.51-1.82 (m, 4H), 1.92-2.11 (m, 1H), 2.63 (t, J=10.9 Hz, 1H), 2.80 (td, J=12.4, 2.8 Hz, 1H), 3.00 (t, J=4.5 Hz, 4H), 3.05-3.24 (m, 2H), 3.53-3.79 (m, 4H), 3.85 (d, J=4.6 Hz, 4H), 5.02 (s, 2H), 5.67 (dd, J=8.9, 3.3 Hz, 1H), 6.29-6.48 (m, 4H), 6.86 (d, J=8.7 Hz, 1H), 6.98 (s, 1H), 7.98 (d, J=5.6 Hz, 1H).

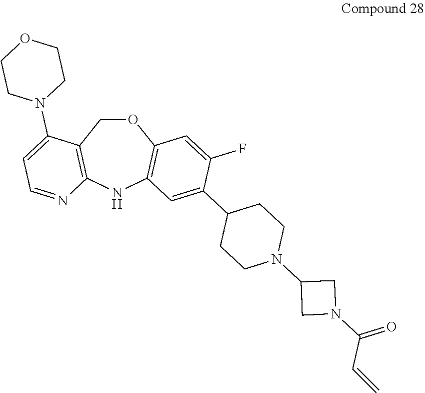 [SMILES: C=CC(=O)N1CC(N2CCC(c3cc4c(cc3F)OCc3c(N5CCOCC5)ccnc3N4)CC2)C1]

Compound 28 was synthesized in a similar manner as Compound 263 using Intermediate 226 instead of Intermediate 57.

LC MS: confirms the MW (RT: 1.55, [M+H]⁺: 494.2, Method: 2).

¹H NMR (300 MHz, Chloroform-d) δ (ppm) 1.65-1.90 (m, 4H), 1.99 (ddd, J=14.5, 10.1, 3.0 Hz, 2H), 2.76 (td, J=9.9, 7.9, 5.9 Hz, 1H), 2.86-3.00 (m, 2H), 3.03 (t, J=4.5 Hz, 4H), 3.13-3.29 (m, 1H), 3.86 (t, J=4.5 Hz, 4H), 3.91-4.03 (m, 1H), 4.05-4.18 (m, 2H), 4.23 (t, J=7.9 Hz, 1H), 5.04 (s, 2H), 5.66 (dd, J=10.1, 2.1 Hz, 1H), 6.11-6.25 (m, 1H), 6.28-6.42 (m, 2H), 6.55-6.69 (m, 2H), 6.78 (s, 1H), 7.98 (d, J=5.6 Hz, 1H).

MP: 238.4° C. (Mettler Toledo MP50), uncorrected.

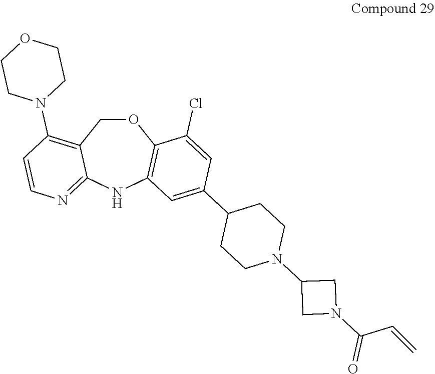 [SMILES: C=CC(=O)N1CC(N2CCC(c3cc(Cl)c4c(c3)Nc3nccc(N5CCOCC5)c3CO4)CC2)C1]

Triethylamine (1.8 mL, 12.6 mmol, 10.0 eq.) was added to a solution of Intermediate 229 (574 mg, 1.26 mmol) in DCM (15 mL). The mixture was cooled in an ice bath. Acryloyl chloride (102 μL, 1.26 mmol) in DCM (10 mL) was added dropwise and the reaction mixture was stirred at room temperature for 2 h. The reaction mixture was quenched by addition of saturated aqueous NaHCO₃ and extracted with DCM. The organic layer was dried over anhydrous MgSO₄, filtered, and concentrated. The residue was purified by flash column chromatography (DCM//DCM/MeOH (9:1) from 100/0 to 0/100) to afford a yellowish solid. A second purification was performed with reverse phase column chromatography (Phenomenex Gemini C18 30×100 mm 5 μm; gradient from 50% [25 mM NH₄HCO₃]−50% [ACN:MeOH (1:1)] to 25% [25 mM NH₄HCO₃]−75% [ACN:MeOH (1:1)]) to yield Compound 29 (225 mg, yield: 39%) as a white solid.

LC MS: confirms the MW (RT: 1.727, [M+H]⁺: 510.2, Method: 3).

¹H NMR (300 MHz, Chloroform-d) δ (ppm) 1.70 (qd, J=12.2, 3.5 Hz, 2H), 1.85 (d, J=12.3 Hz, 2H), 1.89-2.02 (m, 2H), 2.41 (tt, J=12.1, 3.9 Hz, 1H), 2.84-2.96 (m, 2H), 2.99 (t, J=4.5 Hz, 4H), 3.12-3.26 (m, 1H), 3.87 (t, J=4.5 Hz, 4H), 3.96 (dd, J=10.5, 5.5 Hz, 1H), 4.04-4.18 (m, 2H), 4.23 (t, J=7.9 Hz, 1H), 5.11 (s, 2H), 5.66 (dd, J=10.1, 2.1 Hz, 1H), 6.19 (dd, J=17.0, 10.1 Hz, 1H), 6.33 (dd, J=17.0, 2.1 Hz, 1H), 6.41 (d, J=5.6 Hz, 1H), 6.56 (d, J=2.0 Hz, 1H), 6.78 (d, J=2.0 Hz, 1H), 6.95 (s, 1H), 8.01 (d, J=5.6 Hz, 1H).

MP: >300° C. (Mettler Toledo MP50), uncorrected.

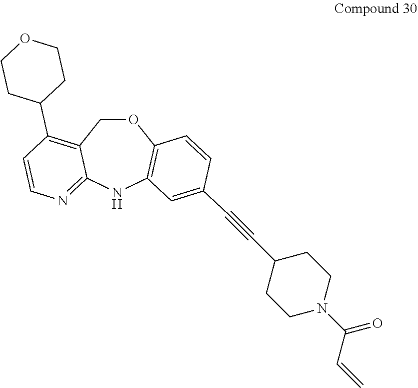 [SMILES: C=CC(=O)N1CCC(C#Cc2ccc3c(c2)Nc2nccc(C4CCOCC4)c2CO3)CC1]

TFA (0.25 mL, 3.3 mmol, 60.0 eq.) was added to a solution of Intermediate 232 (32 mg, 0.054 mmol) in DCM (0.25 mL). The solution was stirred for 30 min at room temperature. the solvent was evaporated and the residue was taken up with 0.5 mL of DCM. Et₃N (75 μL, 0.54 mmol, 10.0 eq.) and acryloyl chloride (5.4 mg, 0.06 mmol, 1.1 eq.) were added at 0° C. and the reaction mixture was stirred for 30 min at room temperature. Then saturated NaHCO₃ was added and the mixture was extracted with more DCM. The organic layer was separated, dried (Na₂SO₄), filtered, and the solvent evaporated in vacuo to yield a yellow oil. The residue was purified by reverse phase HPLC (Stationary phase: C18 XBridge 30×100 mm 5 μm, Mobile phase: Gradient from 67% NH₄HCO₃ 0.25% solution in Water, 33% CH₃CN to 50% NH₄HCO₃ 0.25% solution in Water, 50% CH₃CN) to afford Compound 30 (11 mg, yield: 44%) as a clear oil that solidified upon standing.

LCMS: confirms the MW (RT: 2.16, [M+H]⁺: 444.2, Method: 8).

¹H NMR (400 MHz, Chloroform-d) δ (ppm) 1.60-1.98 (m, 1H) 2.01 (s, 1H) 2.90 (tt, J=7.72, 3.96 Hz, 1H) 3.08 (tt, J=11.85, 3.41 Hz, 1H) 3.37-3.64 (m, 4H) 3.80 (br s, 1H) 3.98 (br s, 1H) 4.10 (dd, J=11.21, 3.81 Hz, 2H) 5.09 (s, 2H) 5.69 (dd, J=10.52, 1.96 Hz, 1H) 6.28 (dd, J=16.88, 1.85 Hz, 1H) 6.59 (dd, J=16.76, 10.52 Hz, 1H) 6.69 (d, J=5.32 Hz, 1H) 6.78-6.88 (m, 3H) 6.90 (s, 1H) 8.08 (d, J=5.32 Hz, 1H).

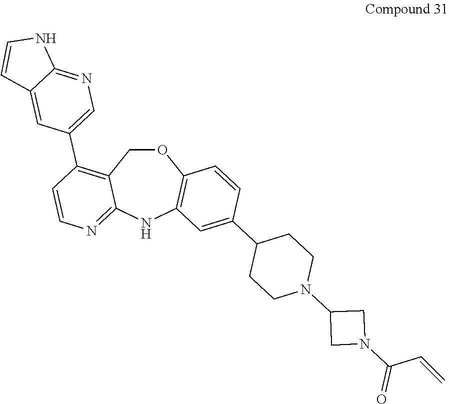 [SMILES: C=CC(=O)N1CC(N2CCC(c3ccc4c(c3)Nc3nccc(-c5cnc6[nH]ccc6c5)c3CO4)CC2)C1]

Compound 31 was synthesized in a similar manner as Compound 4 using Intermediate 234 instead of Intermediate 15.

LCMS: confirms the MW (RT: 2.49, [M+H]⁺: 507.3, Method: 1).

¹H NMR: (400 MHz, DMSO-d₆, 23° C.): δ (ppm) 11.87 (br s, 1H), 9.26 (s, 1H), 8.18 (d, J=2.0 Hz, 1H), 8.14 (d, J=5.1 Hz, 1H), 7.95 (d, J=2.0 Hz, 1H), 7.54-7.61 (m, 1H), 7.15 (d, J=2.0 Hz, 1H), 6.74-6.81 (m, 2H), 6.59 (dd, J=8.6, 2.0 Hz, 1H), 6.54 (dd, J=3.3, 1.8 Hz, 1H), 6.31 (dd, J=17.2, 10.1 Hz, 1H), 6.06-6.15 (m, 1H), 5.63-5.72 (m, 1H), 4.91 (s, 2H), 4.24 (br t, J=8.3 Hz, 1H), 4.04 (br dd, J=9.1, 5.6 Hz, 2H), 3.95 (br dd, J=10.1, 7.6 Hz, 2H), 3.76 (br d, J=5.1 Hz, 2H), 3.09 (br dd, J=3.5, 2.0 Hz, 1H), 2.85-2.96 (m, 2H), 1.85-1.96 (m, 2H), 1.71-1.80 (m, 2H), 1.50-1.67 ppm (m, 1H).

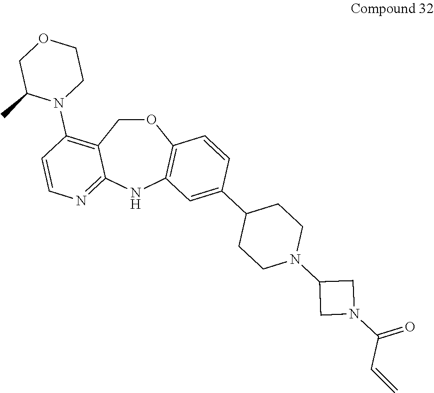 [SMILES: C=CC(=O)N1CC(N2CCC(c3ccc4c(c3)Nc3nccc(N5CCOC[C@@H]5C)c3CO4)CC2)C1]

(*S), pure stereoisomer but absolute stereochemistry undetermined Compound 32 was synthesized in a similar manner as Compound 4 using Intermediate 238 instead of Intermediate 15.

¹H NMR (400 MHz, DMSO-d₆, 23° C.): δ (ppm) 9.01 (s, 1H), 8.00 (d, J=5.6 Hz, 1H), 7.08 (d, J=2.0 Hz, 1H), 6.81 (d, J=8.1 Hz, 1H), 6.52-6.60 (m, 2H), 6.31 (dd, J=16.9, 10.4 Hz, 1H), 6.10 (dd, J=16.9, 2.3 Hz, 1H), 5.67 (dd, J=10.4, 2.3 Hz, 1H), 5.15 (br d, J=13.6 Hz, 1H), 4.89 (d, J=13.6 Hz, 1H), 4.24 (br t, J=8.1 Hz, 1H), 4.01-4.06 (m, 1H), 3.94 (br dd, J=10.1, 7.6 Hz, 1H), 3.82 (br dd, J=10.9, 2.8 Hz, 1H), 3.67-3.78 (m, 3H), 3.38 (br s, 1H), 3.11-3.21 (m, 2H), 2.95-3.03 (m, 1H), 2.84-2.94 (m, 2H), 2.64-2.71 (m, 2H), 2.37 (s, 1H), 1.83-1.96 (m, 2H), 1.73 (br d, J=13.1 Hz, 2H), 1.54-1.62 (m, 2H), 0.84 ppm (d, J=6.1 Hz, 3H)

LCMS: confirms the MW (RT: 2.69, [M+H]⁺: 490.3, Method: 1).

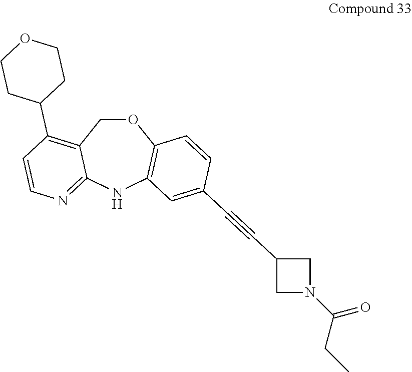 [SMILES: CCC(=O)N1CC(C#Cc2ccc3c(c2)Nc2nccc(C4CCOCC4)c2CO3)C1]

Compound 33 was synthesized in a similar manner as Compound 30 using Intermediate 239 instead of Intermediate 232.

LCMS: confirms the MW (RT: 1.95, [M+H]⁺: 416.2, Method: 4).

¹H NMR (500 MHz, DMSO-d₆) δ (ppm) 1.48-1.76 (m, 4H) 3.19 (tt, J=11.70, 3.47 Hz, 1H) 3.53 (td, J=11.56, 1.73 Hz, 2H) 3.75 (tt, J=8.81, 6.21 Hz, 1H) 3.82-4.02 (m, 3H) 4.19-4.24 (m, 1H) 4.27 (t, J=9.10 Hz, 1H) 4.55 (t, J=8.53 Hz, 1H) 5.10 (s, 2H) 5.61-5.78 (m, 1H) 6.12 (dd, J=17.05, 2.02 Hz, 1H) 6.32 (dd, J=17.05, 10.40 Hz, 1H) 6.69-6.80 (m, 2H) 6.85 (d, J=8.38 Hz, 1H) 7.30 (d, J=2.02 Hz, 1H) 8.07 (d, J=5.20 Hz, 1H) 9.21 (s, 1H).

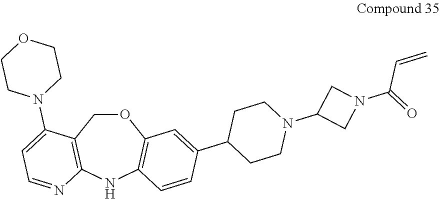 [SMILES: C=CC(=O)N1CC(N2CCC(c3ccc4c(c3)OCc3c(N5CCOCC5)ccnc3N4)CC2)C1]

Compound 35 was synthesized in a similar manner as Compound 173 using Intermediate 247 instead of Intermediate 65.

LCMS: confirms the MW (RT: 1.304, [M+H]⁺: 476.1, Method: 2).

MP: 117.9° C. (Mettler Toledo FP62), uncorrected.

¹H NMR (300 MHz, Chloroform-d) δ (ppm) 7.97 (d, J=5.6 Hz, 1H), 6.85-6.74 (m, 4H), 6.43-6.27 (m, 2H), 6.19 (dd, J=17.0, 10.1 Hz, 1H), 5.66 (d, J=10.1 Hz, 1H), 5.06 (s, 2H), 4.23 (t, J=7.7 Hz, 1H), 4.15-4.07 (m, 2H), 4.04-3.92 (m, 1H), 3.91-3.84 (m, 4H), 3.24-3.15 (m, 1H), 3.08-303 (m, 4H), 2.99-2.85 (m, 2H), 2.49-2.36 (m, 1H), 1.96 (t, J=11.1 Hz, 2H), 1.85 (d, J=12.2 Hz, 2H), 1.80-1.67 (m, 2H).

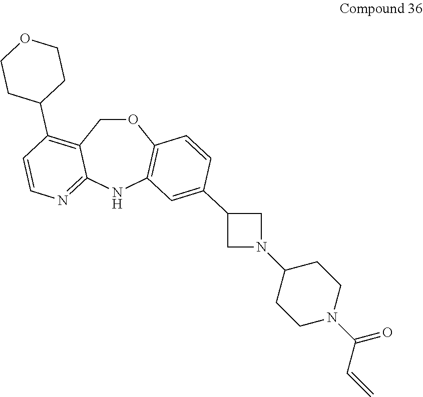 [SMILES: C=CC(=O)N1CCC(N2CC(c3ccc4c(c3)Nc3nccc(C5CCOCC5)c3CO4)C2)CC1]

Compound 36 was synthesized in a similar manner as Compound 30 using Intermediate 249 instead of Intermediate 232.

LCMS: confirms the MW (RT: 1.55, [M+H]⁺: 475.3, Method: 8).

¹H NMR (400 MHz, Chloroform-d) δ (ppm) 1.13 (d, J=6.36 Hz, 1H) 1.33 (dtd, J=13.15, 9.39, 9.39, 3.90 Hz, 2H) 1.62-1.92 (m, 9H) 2.28-2.39 (m, 1H) 3.02-3.15 (m, 4H) 3.21 (br t, J=10.84 Hz, 1H) 3.50-3.63 (m, 3H) 3.70 (t, J=7.51 Hz, 2H) 3.88 (br d, J=12.72 Hz, 1H) 4.10 (dd, J=11.13, 3.90 Hz, 2H) 4.25 (br d, J=12.72 Hz, 1H) 5.09 (s, 2H) 5.30 (s, 1H) 5.64-5.69 (m, 1H) 6.25 (dd, J=16.91, 1.88 Hz, 1H) 6.58 (dd, J=16.91, 10.55 Hz, 1H) 6.65-6.72 (m, 2H) 6.75 (d, J=2.02 Hz, 1H) 6.89 (d, J=8.09 Hz, 1H) 7.00 (s, 1H) 7.45-7.53 (m, 1H) 8.07 (d, J=5.49 Hz, 1H).

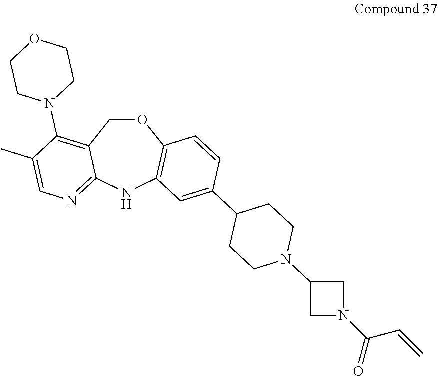 [SMILES: C=CC(=O)N1CC(N2CCC(c3ccc4c(c3)Nc3ncc(C)c(N5CCOCC5)c3CO4)CC2)C1]

Compound 37 was synthesized in a similar manner as Compound 3 using Intermediate 259 instead of Intermediate 10.

LCMS: confirms the MW (RT: 2.65, [M+H]⁺: 490.3, Method: 1).

¹H NMR (500 MHz, DMSO-d₆, 22° C.): δ (ppm) 8.85 (s, 1H), 7.81 (s, 1H), 7.04 (d, J=2.2 Hz, 1H), 6.80 (d, J=8.2 Hz, 1H), 6.55 (dd, J=8.4, 2.0 Hz, 1H), 6.27-6.35 (m, 1H), 6.10 (dd, J=16.9, 2.4 Hz, 1H), 5.63-5.70 (m, 1H), 5.08 (s, 2H), 4.18-4.31 (m, 1H), 4.03 (br dd, J=8.8, 5.0 Hz, 1H), 3.94 (dd, J=10.1, 7.6 Hz, 1H), 3.65-3.77 (m, 5H), 2.99-3.18 (m, 5H), 2.78-2.99 (m, 3H), 2.31-2.39 (m, 1H), 2.20 (s, 3H), 1.84-1.92 (m, 2H), 1.73 (br d, J=11.7 Hz, 2H), 1.52-1.62 (m, 2H), 1.42 ppm (s, 1H)

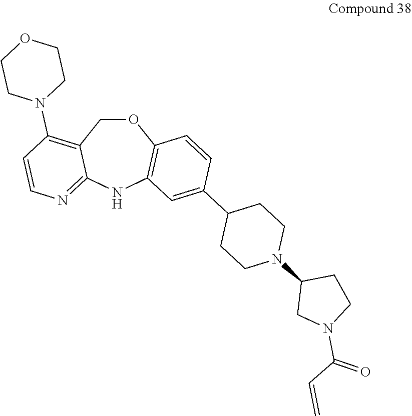 [SMILES: C=CC(=O)N1CC[C@H](N2CCC(c3ccc4c(c3)Nc3nccc(N5CCOCC5)c3CO4)CC2)C1]

(*S), pure stereoisomer but absolute stereochemistry undetermined Compound 38 was synthesized in a similar manner as Compound 3 using Intermediate 261 instead of Intermediate 10.

LCMS: confirms the MW (RT: 2.55, [M+H]⁺: 490.3, Method: 1).

¹H NMR (500 MHz, DMSO-d₆, 22° C.): δ (ppm) 8.92 (d, J=2.5 Hz, 1H), 7.97 (d, J=5.4 Hz, 1H), 7.08 (d, J=2.2 Hz, 1H), 6.80 (d, J=7.9 Hz, 1H), 6.54-6.63 (m, 2H), 6.44 (d, J=5.7 Hz, 1H), 6.13 (ddd, J=16.8, 4.5, 2.4 Hz, 1H), 5.66 (ddd, J=10.2, 7.6, 2.4 Hz, 1H), 4.96 (s, 2H), 3.88 (dd, J=10.1, 7.3 Hz, 1H), 3.71-3.78 (m, 5H), 3.56-3.62 (m, 1H), 3.49 (td, J=10.2, 6.8 Hz, 1H), 3.22-3.30 (m, 1H), 3.00-3.09 (m, 2H), 2.83-2.94 (m, 6H), 2.74-2.81 (m, 1H), 2.32-2.39 (m, 1H), 2.02-2.18 (m, 3H), 1.74-1.81 (m, 1H), 1.71 (br d, J=7.3 Hz, 2H), 1.53-1.68 ppm (m, 3H)

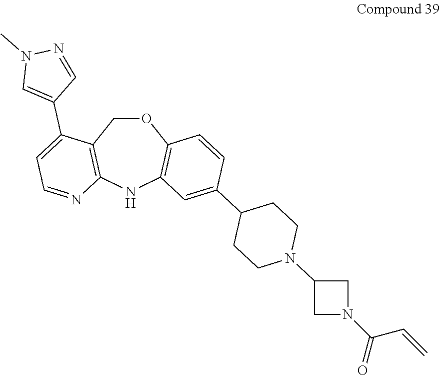 [SMILES: C=CC(=O)N1CC(N2CCC(c3ccc4c(c3)Nc3nccc(-c5cnn(C)c5)c3CO4)CC2)C1]

Compound 39 was synthesised in a similar manner as Compound 164 using Intermediate 264 instead of Intermediate 50.

LCMS: confirms the MW (RT: 3.606, [M+H]⁺: 471.2, Method: 9).

¹H NMR (300 MHz, Chloroform-d) δ (ppm) 8.05 (d, J=5.1 Hz, 1H), 7.59 (s, 1H), 7.51 (s, 1H), 6.92-6.87 (m, 2H), 6.69-6.64 (m, 3H), 6.33 (d, J=16.9 Hz, 1H), 6.20 (dd, J=17.0, 10.1 Hz, 1H), 5.66 (d, J=10.1 Hz, 1H), 5.11 (s, 2H), 4.24 (t, J=7.6 Hz, 1H), 4.16-4.08 (m, 2H), 3.9 (s, 3H), 4.02-3.95 (m, 1H), 3.28-3.15 (m, 1H), 3.04-2.86 (m, 2H), 2.51-2.39 (m, 1H), 1.97 (t, J=11.4 Hz, 2H), 1.87 (d, J=12.5 Hz, 2H), 1.76 (t, J=11.2 Hz, 2H).

MP: 211.0° C. (Mettler Toledo FP62), 10° C./min, uncorrected.

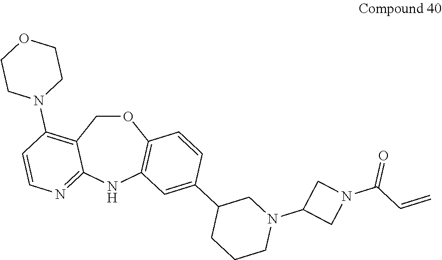 [SMILES: C=CC(=O)N1CC(N2CCCC(c3ccc4c(c3)Nc3nccc(N5CCOCC5)c3CO4)C2)C1]

Compound 40 was synthesized in a similar manner as Compound 29 using Intermediate 266 instead of Intermediate 229.

LCMS: confirms the MW (RT: 1.439, [M+H]⁺: 476.2, Method: 2).

¹H NMR (400 MHz, Chloroform-d) δ (ppm) 1.58-2.00 (m, 6H), 2.62-2.77 (m, 1H), 2.86 (1, J=13.1 Hz, 2H), 3.02 (t, J=4.5 Hz, 4H), 3.18 (q, J=6.4 Hz, 1H), 3.87 (t, J=4.6 Hz, 4H), 3.90-4.31 (m, 4H), 5.05 (s, 2H), 5.59-5.70 (m, 1H), 6.17 (ddd, J=16.8, 10.1, 5.8 Hz, 1H), 6.25-6.43 (m, 2H), 6.57-6.71 (m, 2H), 6.82-6.93 (m, 2H), 7.99 (d, J=5.6 Hz, 1H).

MP: 141.3° C. (Mettler Toledo MP50), uncorrected.

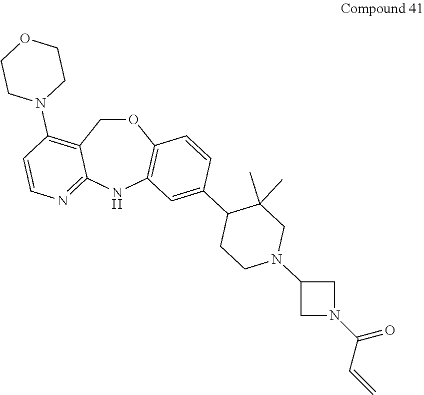 [SMILES: C=CC(=O)N1CC(N2CCC(c3ccc4c(c3)Nc3nccc(N5CCOCC5)c3CO4)C(C)(C)C2)C1]

Compound 41 was synthesized in a similar manner as Compound 29 using Intermediate 269 instead of Intermediate 229.

LCMS: confirms the MW (RT: 1.577, [M+H]⁺: 504.3, Method: 2).

¹H NMR (400 MHz, Chloroform-d) δ (ppm) 0.75 (s, 3H), 0.90 (s, 3H), 1.57 (d, J=12.9 Hz, 1H), 1.73 (dd, J=10.9, 4.6 Hz, 1H), 1.87 (t, J=11.2 Hz, 1H), 2.08 (qd, J=12.5, 3.6 Hz, 1H), 2.24 (dd, J=12.9, 3.2 Hz, 1H), 2.36-2.48 (m, 1H), 2.86-2.99 (m, 1H), 3.03 (t, J=4.5 Hz, 4H), 3.13 (t, J=6.1 Hz, 1H), 3.87 (t, J=4.5 Hz, 4H), 3.91-4.28 (m, 4H), 5.06 (s, 2H), 5.60-5.74 (m, 1H), 6.21 (ddd, J=17.0, 10.1, 1.9 Hz, 1H), 6.29-6.46 (m, 2H), 6.54-6.65 (m, 2H), 6.77-6.92 (m, 2H), 7.98 (d, J=5.6 Hz, 1H).

MP: 154.7° C. (Mettler Toledo MP50), uncorrected.

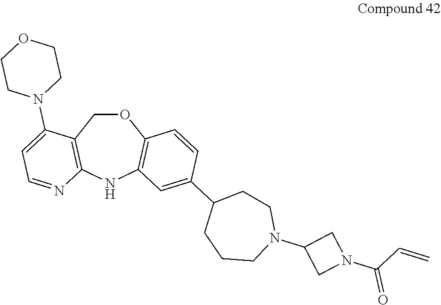 [SMILES: C=CC(=O)N1CC(N2CCCC(c3ccc4c(c3)Nc3nccc(N5CCOCC5)c3CO4)CC2)C1]

Compound 42 was synthesized in a similar manner as Compound 272 using Intermediate 272 instead of Intermediate 89.

LCMS: confirms the MW (RT: 1.498, [M+H]⁺: 490.1, Method: 2).

¹H NMR (300 MHz, Chloroform-d) δ (ppm) 7.96 (d, J=5.7 Hz, 1H), 7.04 (br s, 1H), 6.87 (d, J=8.0 Hz, 1H), 6.65 (s, 1H), 6.63 (d, J=8.1 Hz, 1H), 6.41-6.29 (m, 2H), 6.20 (dd, J=17.0, 10.1 Hz, 1H), 5.66 (d, J=10.2 Hz, 1H), 5.04 (s, 2H), 4.22 (t, J=7.5 Hz, 1H), 4.10-4.01 (m, 2H), 3.99-3.79 (m, 5H), 3.51-3.43 (m, 1H), 3.07-3.00 (m, 4H), 2.76-2.50 (m, 5H), 2.04-1.67 (m, 6H).

MP: 177.9° C. (Mettler Toledo FP62), uncorrected.

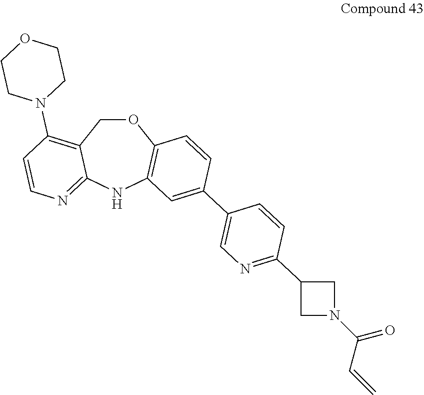 [SMILES: C=CC(=O)N1CC(c2ccc(-c3ccc4c(c3)Nc3nccc(N5CCOCC5)c3CO4)cn2)C1]

Compound 43 was synthesized in a similar manner as Compound 19 using Intermediate 273B instead of Intermediate 197.

LCMS: confirms the MW (RT: 2.064, [M+H]⁺: 470.1, Method: 2).

¹H NMR (300 MHz, Chloroform-d) δ (ppm) 3.06 (t, J=4.5 Hz, 4H), 3.89 (t, J=4.6 Hz, 4H), 3.98-4.09 (m, 1H), 4.31 (dd, J=10.1, 6.3 Hz, 1H), 4.43-4.67 (m, 3H), 5.11 (s, 2H), 5.69 (dd, J=9.9, 2.3 Hz, 1H), 6.17-6.38 (m, 2H), 6.39-6.44 (m, 1H), 6.94-7.08 (m, 4H), 7.28 (s, 1H), 7.78 (dd, J=8.1, 2.3 Hz, 1H), 8.02 (d, J=5.7 Hz, 1H), 8.78 (s, 1H).

MP: 245.1° C. (Mettler Toledo MP50), uncorrected.

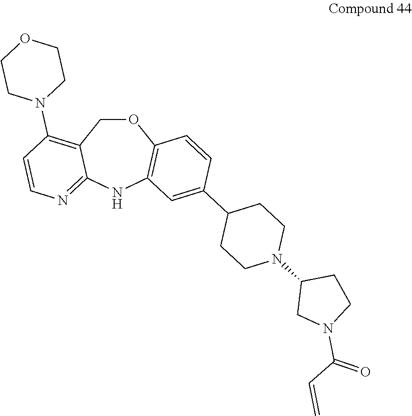 [SMILES: C=CC(=O)N1CC[C@@H](N2CCC(c3ccc4c(c3)Nc3nccc(N5CCOCC5)c3CO4)CC2)C1]

(*R), pure stereoisomer but absolute stereochemistry undetermined Compound 44 was synthesized in a similar manner as Compound 3 using Intermediate 274 instead of Intermediate 10.

LCMS: confirms the MW (RT: 2.54, [M+H]⁺: 490.3, Method: 1).

¹H NMR (500 MHz, DMSO-d₆, 27° C.): δ (ppm) 8.89 (d, J=1.9 Hz, 1H), 7.97 (d, J=5.7 Hz, 1H), 7.08 (d, J=1.9 Hz, 1H), 6.80 (d, J=8.2 Hz, 1H), 6.53-6.65 (m, 2H), 6.43 (d, J=5.4 Hz, 1H), 6.09-6.17 (m, 1H), 5.65 (ddd, J=10.2, 7.6, 2.4 Hz, 1H), 4.96 (s, 2H), 3.87 (br dd, J=9.9, 7.4 Hz, 1H), 3.70-3.80 (m, 5H), 3.55-3.65 (m, 1H), 3.49 (td, J=10.1, 6.6 Hz, 1H), 3.22-3.28 (m, 1H), 2.96-3.10 (m, 2H), 2.82-2.95 (m, 6H), 2.75-2.82 (m, 1H), 2.54-2.59 (m, 1H), 2.31-2.40 (m, 1H), 2.01-2.18 (m, 3H), 1.75-1.85 (m, 1H), 1.71 (br s, 2H), 1.53-1.69 ppm (m, 3H) MP: 110.42° C./−31.57 J/g (DSC: 25° C. to 350° C./10° C. min/40 μl Al).

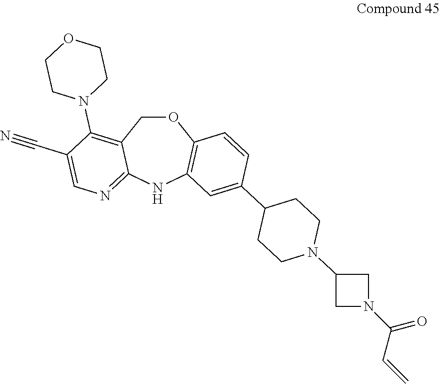 [SMILES: C=CC(=O)N1CC(N2CCC(c3ccc4c(c3)Nc3ncc(C#N)c(N5CCOCC5)c3CO4)CC2)C1]

Compound 45 was synthesized in a similar manner as Compound 3 using Intermediate 277 instead of Intermediate 10.

LCMS: confirms the MW (RT: 2.58, [M+H]⁺: 501.3, Method: 1).

¹H NMR: (500 MHz, DMSO-d₆, 28° C.): δ (ppm) 9.74 (s, 1H), 8.31 (s, 1H), 7.16 (d, J=1.9 Hz, 1H), 6.89 (d, J=8.2 Hz, 1H), 6.72 (dd, J=8.4, 2.0 Hz, 1H), 6.31 (dd, J=16.9, 10.2 Hz, 1H), 6.10 (dd, J=17.0, 2.2 Hz, 1H), 5.66 (dd, J=10.1, 2.2 Hz, 1H), 4.99 (s, 2H), 4.24 (t, J=8.0 Hz, 1H), 4.03 (br dd, J=8.8, 5.4 Hz, 1H), 3.94 (dd, J=10.1, 7.3 Hz, 1H), 3.74-3.76 (m, 1H), 3.71-3.73 (m, 1H), 3.28 (br s, 3H), 3.10-3.17 (m, 1H), 2.84-2.93 (m, 2H), 2.52-2.53 (m, 1H), 2.41 (ddd, J=12.1, 8.4, 3.8 Hz, 2H), 1.85-1.95 (m, 2H), 1.74 (br d, J=12.9 Hz, 2H), 1.57 ppm (qd, J=12.2, 3.2 Hz, 2H)

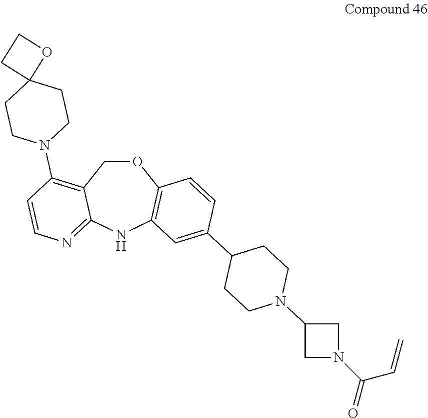 [SMILES: C=CC(=O)N1CC(N2CCC(c3ccc4c(c3)Nc3nccc(N5CCC6(CCO6)CC5)c3CO4)CC2)C1]

Compound 46 was synthesized in a similar manner as Compound 3 using Intermediate 281 instead of Intermediate 10.

confirms the MW (RT: 2.51, [M+H]⁺: 516.6, Method: 10).

¹H NMR: (500 MHz, DMSO-d₆, 22° C.): δ (ppm) 8.90 (br s, 1H), 7.93 (d, J=5.4 Hz, 1H), 7.07 (d, J=1.9 Hz, 1H), 6.82 (br d, J=8.5 Hz, 1H), 6.57 (dd, J=8.4, 2.0 Hz, 1H), 6.42 (d, J=5.7 Hz, 1H), 6.31 (dd, J=17.0, 10.1 Hz, 1H), 6.11 (br d, J=17.0 Hz, 1H), 5.67 (br d, J=9.5 Hz, 1H), 4.93 (s, 2H), 4.42 (t, J=7.7 Hz, 2H), 4.24 (br s, 1H), 4.04 (br s, 1H), 3.95 (br s, 1H), 3.68-3.80 (m, 1H), 3.14 (br s, 1H), 2.88-2.99 (m, 3H), 2.78-2.88 (m, 3H), 2.52-2.53 (m, 1H), 2.31-2.46 (m, 4H), 1.82-1.99 (m, 6H), 1.73 (br d, J=6.3 Hz, 2H), 1.59 (br s, 2H), 1.16-1.30 ppm (m, 1H)

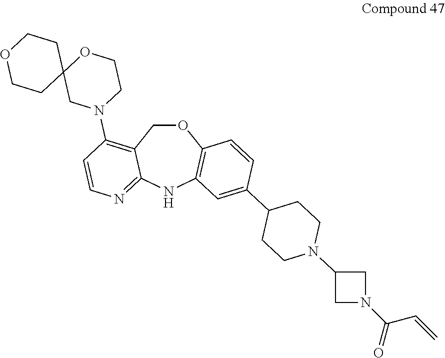 [SMILES: C=CC(=O)N1CC(N2CCC(c3ccc4c(c3)Nc3nccc(N5CCOC6(CCOCC6)C5)c3CO4)CC2)C1]

Compound 47 was synthesized in a similar manner as Compound 3 using Intermediate 283 instead of Intermediate 10.

LCMS: confirms the MW (RT: 2.58, [M+H]⁺: 546.4, Method: 1).

¹H NMR: (500 MHz, DMSO-d₆, 23° C.): δ (ppm) 8.95 (s, 1H), 7.97 (d, J=5.7 Hz, 1H), 7.08 (d, J=1.9 Hz, 1H), 6.83 (d, J=8.2 Hz, 1H), 6.59 (dd, J=8.2, 1.9 Hz, 1H), 6.44 (d, J=5.4 Hz, 1H), 6.31 (dd, J=17.0, 10.4 Hz, 1H), 6.10 (dd, J=17.0, 2.2 Hz, 1H), 5.67 (dd, J=10.1, 2.2 Hz, 1H), 5.01 (s, 2H), 4.24 (t, J=8.2 Hz, 1H), 4.03 (br dd, J=8.7, 4.9 Hz, 1H), 3.91-3.98 (m, 1H), 3.79-3.85 (m, 2H), 3.74 (br dd, J=10.1, 5.0 Hz, 1H), 3.57-3.65 (m, 4H), 3.11-3.16 (m, 1H), 2.84-2.93 (m, 4H), 2.77 (s, 2H), 2.34-2.42 (m, 2H), 1.88 (br d, J=15.1 Hz, 4H), 1.73 (br d, J=12.0 Hz, 2H), 1.51-1.68 ppm (m, 4H)

MP: 196° C. (DSC: 25° C. to 300° C./10° C. min/40 μl Al).

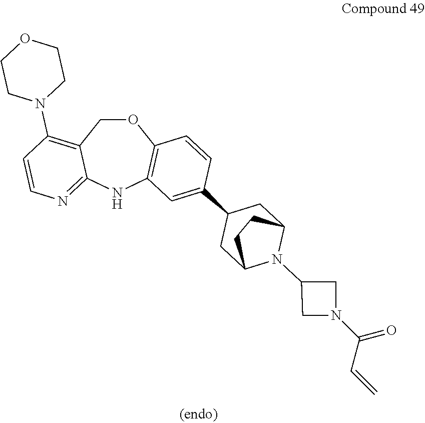 [SMILES: C=CC(=O)N1CC(N2[C@@H]3CC[C@H]2C[C@@H](c2ccc4c(c2)Nc2nccc(N5CCOCC5)c2CO4)C3)C1]

Triethylamine (0.5 mL, 3.8 mmol, 15.0 eq.) was added to a solution of Intermediate 289 (116 mg, 0.26 mmol, 1.0 eq.) in DCM (4 mL). The mixture was cooled in an ice bath. Then acryloyl chloride (21 μL, 0.26 mmol, 1.0 eq.) in DCM (2 mL) was added dropwise and the reaction mixture was stirred at room temperature for 2 h. The reaction mixture was quenched by addition of saturated aqueous NaHCO₃ and extracted with DCM. The organic layer was dried over anhydrous MgSO₄ and concentrate. The residue was purified by flash column chromatography (DCM/DCM:MeOH (9:1) gradient). A second purification was performed by reverse phase column chromatography (Phenomenex Gemini C18 30×100 mm 5 μm; gradient from 81% [25 mM NH₄HCO₃]−19% ACN to 45% [25 mM NH₄HCO₃]−55% ACN). The pure fractions were combined, basified, and extracted with DCM. The organic layer was concentrated to yield Compound 49 (100 mg, yield: 53%).

LCMS: confirms the MW (RT: 1.472, [M+H]⁺: 502.2, Method: 2).

¹H NMR (300 MHz, Chloroform-d) δ (ppm) 1.45-1.61 (m, 4H), 1.80-1.97 (m, 2H), 2.27-2.45 (m, 2H), 2.91-3.09 (m, 5H), 3.09-3.26 (m, 2H), 3.26-3.36 (m, 1H), 3.76-3.91 (m, 5H), 3.91-4.03 (m, 1H), 4.03-4.17 (m, 1H), 4.16-4.28 (m, 1H), 5.05 (s, 2H), 5.60-5.73 (m, 1H), 6.13-6.26 (m, 1H), 6.31 (d, J=2.1 Hz, 1H), 6.37 (d, J=5.5 Hz, 1H), 6.62-6.74 (m, 2H), 6.79 (s, 1H), 6.81-6.89 (m, 1H), 7.99 (d, J=5.6 Hz, 1H).

MP: 231.7° C. (Mettler Toledo MP50), uncorrected.

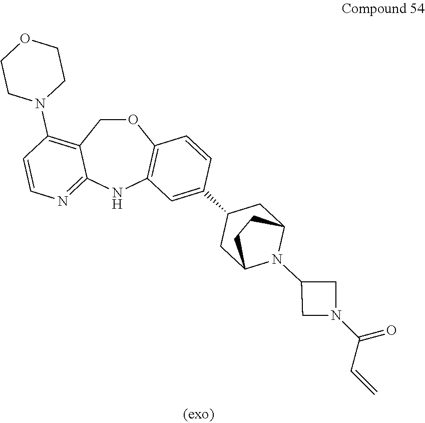 [SMILES: C=CC(=O)N1CC(N2[C@@H]3CC[C@H]2C[C@H](c2ccc4c(c2)Nc2nccc(N5CCOCC5)c2CO4)C3)C1]

Compound 54 was synthesized in a similar manner as Compound 49 using Intermediate 288 instead of Intermediate 289.

LCMS: confirms the MW (RT: 1.478, [M+H]⁺: 502.3, Method: 2).

¹H NMR (300 MHz, Chloroform-d) δ (ppm) 1.58-1.90 (m, 61H), 1.90-2.06 (m, 2H), 2.72-2.88 (m, 1H), 3.02 (t, J=4.6 Hz, 4H), 3.22 (d, J=14.5 Hz, 2H), 3.37-3.51 (m, 1H), 3.78-3.93 (m, 5H), 3.96-4.07 (m, 1H), 4.05-4.19 (m, 1H), 4.19-4.31 (in, 1H), 5.04 (s, 21-H), 5.60-5.71 (m, 1H), 6.13-6.26 (m, 1H), 6.26-6.33 (m, 1H), 6.33-6.40 (m, 1H), 6.60-6.71 (m, 2H), 6.77 (s, 1H), 6.82-6.92 (m, 1H), 7.98 (d, J=5.6 Hz, 1H).

MP: 216.6° C. (Mettler Toledo MP50), uncorrected.

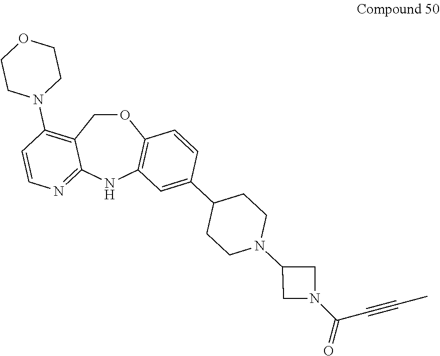 [SMILES: CC#CC(=O)N1CC(N2CCC(c3ccc4c(c3)Nc3nccc(N5CCOCC5)c3CO4)CC2)C1]

Compound 50 was synthesized in a similar manner as Compound 3 using Intermediate 15 instead of Intermediate 10 and 2-butynoic acid [CAS: 590-93-2] instead of acrylic acid.

LCMS: confirms the MW (RT: 2.69, [M+H]⁺: 488.3, Method: 1).

¹H NMR: (500 MHz, DMSO-d₆, 27° C.): δ (ppm) 8.90 (s, 1H), 7.97 (d, J=5.4 Hz, 1H), 7.08 (d, J=1.6 Hz, 1H), 6.80 (d, J=8.2 Hz, 1H), 6.56-6.61 (m, 1H), 6.44 (d, J=5.4 Hz, 1H), 4.97 (s, 2H), 4.14 (br t, J=8.4 Hz, 1H), 3.88-3.97 (m, 2H), 3.73-3.80 (m, 4H), 3.70 (br dd, J=10.1, 5.0 Hz, 1H), 3.31-3.35 (m, 1H), 3.26-3.28 (m, 1H), 3.15 (br t, J=6.1 Hz, 1H), 2.85-2.94 (m, 6H), 2.47 (br s, 1H), 2.23-2.42 (m, 2H), 1.95-2.04 (m, 3H), 1.89 (br t, J=11.2 Hz, 2H), 1.72 (br d, J=12.6 Hz, 2H), 1.49-1.65 ppm (m, 2H).

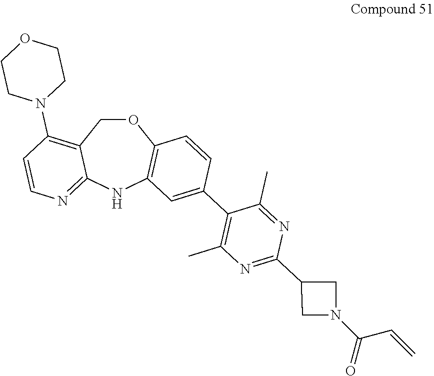 [SMILES: C=CC(=O)N1CC(c2nc(C)c(-c3ccc4c(c3)Nc3nccc(N5CCOCC5)c3CO4)c(C)n2)C1]

Compound 51 was synthesized in a similar manner as Compound 173 using Intermediate 292 instead of Intermediate 65.

LC MS: confirms the MW (RT: 2.136, [M+H]⁺: 499.1, Method: 2)

¹H NMR (300 MHz, Chloroform-d) δ (ppm) 8.01 (d, J=5.6 Hz, 1H), 7.03 (d, J=8.0 Hz, 1H), 6.79 (br s, 1H), 6.57 (s, 1H), 6.55 (d, J=9.0 Hz, 1H), 6.43-6.34 (m, 2H), 6.26 (dd, J=17.0, 9.8 Hz, 1H), 5.68 (dd, J=9.8, 2.1 Hz, 1H), 5.13 (s, 2H), 4.66 (t, J=7.2 Hz, 1H), 4.58 (t, J=8.5 Hz, 1H), 4.52-4.41 (m, 2H), 4.14-4.00 (m, 1H), 3.96-3.82 (m, 4H), 3.14-2.99 (m, 4H), 2.28 (s, 6H).

MP: 242.0° C. (Mettler Toledo FP62), 10° C./min, uncorrected.

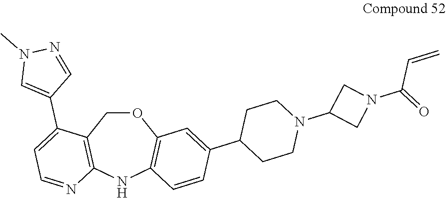 [SMILES: C=CC(=O)N1CC(N2CCC(c3ccc4c(c3)OCc3c(-c5cnn(C)c5)ccnc3N4)CC2)C1]

Compound 52 was synthesized in a similar manner as Compound 173 using Intermediate 295 instead of Intermediate 65.

LC MS: confirms the MW (RT: 1.418, [M+H]⁺: 471.1, Method: 2).

¹H NMR: (300 MHz, Chloroform-d) δ (ppm) 8.04 (d, J=5.1 Hz, 1H), 7.60 (s, 1H), 7.52 (s, 1H), 6.90 (s, 1H), 6.83-6.75 (m, 3H), 6.67 (d, J=5.1 Hz, 1H), 6.39-6.27 (m, 1H), 6.19 (dd, J=17.0, 10.1 Hz, 1H), 5.72-5.59 (m, 1H), 5.12 (s, 2H), 4.23 (t, J=7.9 Hz, 1H), 4.15-4.07 (m, 2H), 3.99 (s, 3H), 4.04-3.91 (m, 1H), 3.24-3.15 (m, 1H), 3.03-2.85 (m, 2H), 2.49-2.38 (m, 1H), 1.94 (t, J=11.3 Hz, 2H), 1.86 (d, J=12.1 Hz, 2H), 1.79-1.68 (m, 2H).

MP: 113.0° C. (Mettler Toledo FP62), uncorrected.

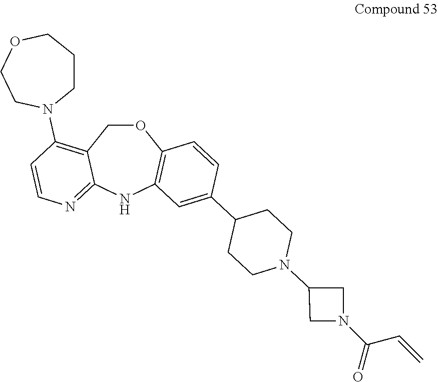 [SMILES: C=CC(=O)N1CC(N2CCC(c3ccc4c(c3)Nc3nccc(N5CCCOCC5)c3CO4)CC2)C1]

Compound 53 was synthesized in a similar manner as Compound 3 using Intermediate 296 instead of Intermediate 10.

LC MS: confirms the MW (RT: 2.58, [M+H]⁺: 490.3, Method: 1).

¹H NMR: (500 MHz, DMSO-d₆, 26° C.): δ (ppm) 8.74 (s, 1H), 7.86 (d, J=5.7 Hz, 1H), 7.07 (d, J=1.9 Hz, 1H), 6.80 (d, J=8.2 Hz, 1H), 6.57 (dd, J=8.2, 1.9 Hz, 1H), 6.43 (d, J=5.7 Hz, 1H), 6.31 (dd, J=16.9, 10.2 Hz, 1H), 6.10 (dd, J=17.0, 2.2 Hz, 1H), 5.64-5.70 (m, 1H), 4.96 (s, 2H), 4.18-4.31 (m, 1H), 4.04 (br dd, J=8.8, 5.0 Hz, 1H), 3.86-3.99 (m, 1H), 3.71-3.81 (m, 5H), 3.27-3.33 (m, 5H), 3.07-3.23 (m, 1H), 2.83-2.95 (m, 2H), 2.33-2.42 (m, 1H), 2.07 (s, 1H), 1.85-1.99 (m, 4H), 1.73 (br d, J=12.6 Hz, 2H), 1.58 ppm (qd, J=12.3, 3.2 Hz, 2H).

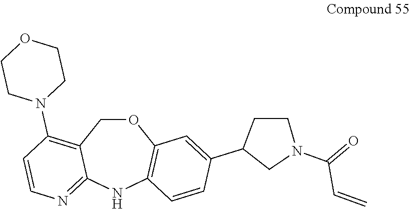 [SMILES: C=CC(=O)N1CCC(c2ccc3c(c2)OCc2c(N4CCOCC4)ccnc2N3)C1]

Compound 55 was synthesized in a similar manner as Compound 262 using Intermediate 299 instead of Intermediate 76.

LC MS: confirms the MW (RT: 1.901, [M+H]⁺: 407.2, Method: 2).

¹H NMR (300 MHz, Chloroform-d) δ (ppm) 2.83-2.12 (m, 1H), 2.19-2.41 (m, 1H), 2.99-3.08 (m, 3H), 3.30 (dt, J=24.5, 8.6 Hz, 1H), 3.45 (t, J=10.1 Hz, 1H), 3.50-3.67 (m, 1H), 3.68-3.82 (m, 1H), 3.87 (t, J=4.6 Hz, 4H), 3.91-4.08 (m, 1H), 5.06 (s, 2H), 5.68 (ddd, J=9.1, 6.4, 3.3 Hz, 1H), 6.28-6.52 (m, 3H), 6.77 (s, 2H), 6.82 (s, 1H), 7.15 (s, 1H), 7.99 (dd, J=5.6, 1.6 Hz, 1H).

MP: 174.8° C. (Mettler Toledo MP50), uncorrected.

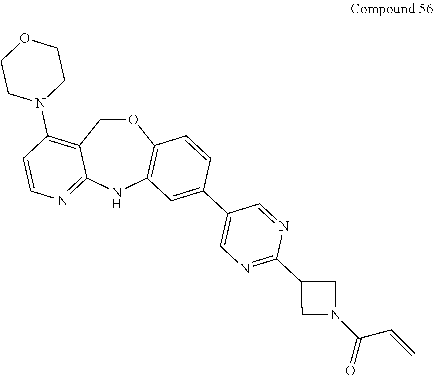 [SMILES: C=CC(=O)N1CC(c2ncc(-c3ccc4c(c3)Nc3nccc(N5CCOCC5)c3CO4)cn2)C1]

Compound 56 was synthesized in a similar manner as Compound 173 using Intermediate 302 instead of Intermediate 65.

LC MS: confirms the MW (RT: 2.001, [M+H]⁺: 471.2, Method: 2).

¹H NMR (300 MHz, Chloroform-d) δ (ppm) 8.86 (s, 2H), 8.03 (d, J=5.6 Hz, 1H), 7.15 (br s, 1H), 7.07 (d, J=8.0 Hz, 1H), 6.99 s, 1H), 6.98 (d, J=8.4 Hz, 1H), 6.47-6.33 (m, 2H), 6.26 (dd, J=17.0, 9.9 Hz, 1H), 5.69 (dd, J=9.9, 2.0 Hz, 1H), 5.12 (s, 2H), 4.72-4.58 (m, 2H), 4.53 (t, J=9.6 Hz, 1H), 4.45-4.34 (m, 1H), 4.22-4.12 (m, 1H), 3.95-3.84 (m, 4H), 3.13-3.01 (m, 4H).

MP: 279.0° C. (Mettler Toledo FP62), 10° C./min; uncorrected.

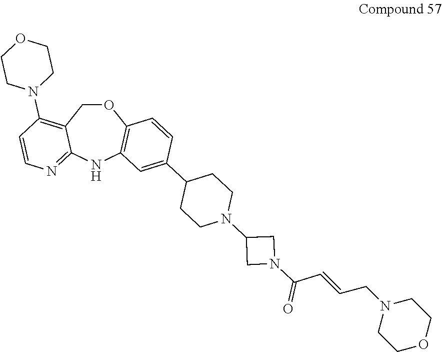 [SMILES: O=C(/C=C/CN1CCOCC1)N1CC(N2CCC(c3ccc4c(c3)Nc3nccc(N5CCOCC5)c3CO4)CC2)C1]

Compound 57 was synthesized in a similar manner as Compound 3 using 4-(morpholin-4-yl)but-2-enoic acid hydrochloride [CAS: 1807940-64-2] instead of acrylic acid and Intermediate 15 instead of Intermediate 10.

LC MS: confirms the MW (RT: 2.43, [M+H]⁺: 575.5, Method: 1).

¹H NMR (500 MHz, DMSO-d₆, 27° C.): δ (ppm) 8.91 (s, 1H), 7.98 (d, J=5.7 Hz, 1H), 7.09 (d, J=1.9 Hz, 1H), 6.81 (d, J=8.2 Hz, 1H), 6.54-6.60 (m, 2H), 6.44 (d, J=5.7 Hz, 1H), 6.13 (d, J=15.1 Hz, 1H), 4.97 (s, 2H), 4.22 (t, J=7.6 Hz, 1H), 3.99-4.04 (m, 1H), 3.90-3.95 (m, 1H), 3.71-3.78 (m, 5H), 3.58 (t, J=4.6 Hz, 4H), 3.06-3.16 (m, 3H), 2.86-2.93 (m, 6H), 2.33-2.41 (m, 6H), 1.90 (br d, J=10.7 Hz, 2H), 1.74 (br d, J=13.2 Hz, 2H), 1.52-1.63 (m, 2H), 1.25 ppm (br s, 2H) MP: 230° C./−77.03 J/g, cristal product (DSC: 25° C. to 350° C./10° C. min/40 μl Al).

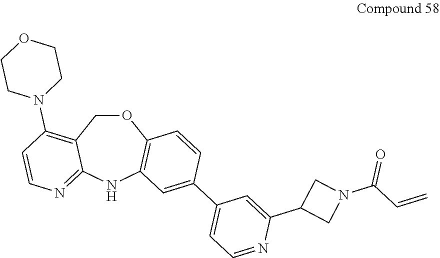 [SMILES: C=CC(=O)N1CC(c2cc(-c3ccc4c(c3)Nc3nccc(N5CCOCC5)c3CO4)ccn2)C1]

Compound 58 was synthesized in a similar manner as Compound 25 using Intermediate 305 instead of Intermediate 218.

LC MS: confirms the MW (RT: 1.880, [M+H]⁺: 470.2, Method: 2).

¹H NMR (300 MHz, Chloroform-d) δ (ppm) 3.07 (t, J=4.6 Hz, 4H), 3.89 (t, J=4.5 Hz, 4H), 3.96-4.10 (m, 1H), 4.33 (dd, J=10.0, 6.3 Hz, 1H), 4.51 (t, J=9.6 Hz, 1H), 4.59 (d, J=7.5 Hz, 2H), 5.11 (s, 2H), 5.68 (dd, J=9.8, 2.2 Hz, 1H), 6.16-6.35 (m, 2H), 6.42 (d, J=5.8 Hz, 1H), 6.99-7.09 (m, 4H), 7.34 (d, J=1.6 Hz, 2H), 8.02 (d, J=5.6 Hz, 1H), 8.63 (d, 1H).

MP: 151.4° C. (Mettler Toledo MP50), uncorrected.

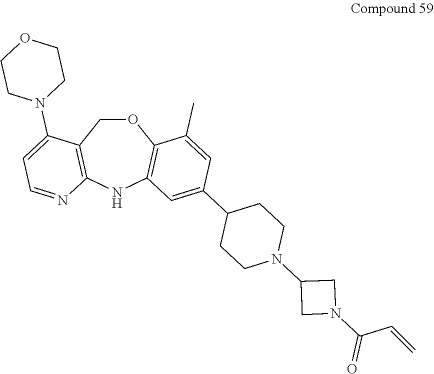 [SMILES: C=CC(=O)N1CC(N2CCC(c3cc(C)c4c(c3)Nc3nccc(N5CCOCC5)c3CO4)CC2)C1]

Compound 59 was synthesized in a similar manner as Compound 263 using Intermediate 307 instead of Intermediate 57.

LC MS: confirms the MW (RT: 1.718, [M+H]⁺: 489.2, Method: 2).

¹H NMR (300 MHz, Chloroform-d) δ (ppm) 1.52-2.03 (m, 10H), 2.24 (s, 3H), 2.32-2.46 (m, 1H), 2.94 (t, J=13.7 Hz, 2H), 3.02-3.15 (m, 1H), 3.20 (h, J=6.4, 5.8 Hz, 1H), 3.51-3.67 (m, 2H), 3.92-4.03 (m, 1H), 4.05-4.17 (m, 4H), 4.24 (t, J=7.9 Hz, 1H), 5.09 (s, 2H), 5.66 (dd, J=10.1, 2.1 Hz, 1H), 6.19 (dd, J=17.0, 10.1 Hz, 1H), 6.33 (dd, J=17.0, 2.1 Hz, 1H), 6.50 (d, J=2.0 Hz, 1H), 6.55 (s, 1H), 6.65 (d, J=5.3 Hz, 1H), 6.85 (s, 1H), 8.05 (d, J=5.3 Hz, 1H).

MP: 231.7° C. (Mettler Toledo MP50), uncorrected.

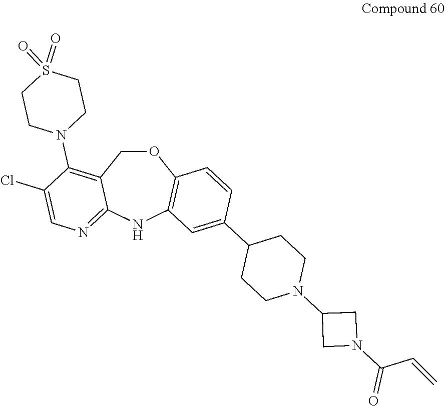 [SMILES: C=CC(=O)N1CC(N2CCC(c3ccc4c(c3)Nc3ncc(Cl)c(N5CCS(=O)(=O)CC5)c3CO4)CC2)C1]

Compound 60 was synthesized in a similar manner as Compound 3 using Intermediate 317 instead of Intermediate 10.

LC MS: confirms the MW (RT: 2.45, [M+H]⁺: 558.3, Method: 1).

¹H NMR: (400 MHz, DMSO-d₆, 25° C.): δ (ppm) 9.28 (s, 1H), 8.06 (s, 1H), 7.09 (d, J=2.0 Hz, 1H), 6.84 (d, J=8.1 Hz, 1H), 6.64 (dd, J=8.3, 1.8 Hz, 1H), 6.31 (dd, J=17.2, 10.1 Hz, 1H), 6.03-6.16 (m, 1H), 5.64-5.69 (m, 1H), 5.13 (s, 2H), 4.24 (br t, J=7.8 Hz, 1H), 4.03 (br dd, J=8.8, 4.8 Hz, 1H), 3.94 (br dd, J=10.1, 7.1 Hz, 1H), 3.74 (br dd, J=10.4, 5.3 Hz, 3H), 3.11-3.18 (m, 2H), 2.82-2.95 (m, 3H), 2.29-2.43 (m, 1H), 1.83-1.96 (m, 2H), 1.73 (br d, J=12.6 Hz, 2H), 1.49-1.65 ppm (m, 2H)

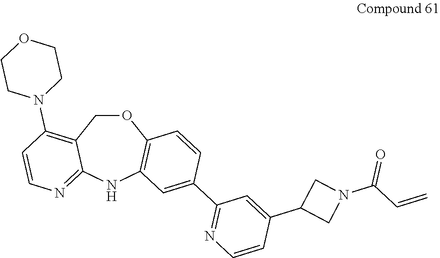 [SMILES: C=CC(=O)N1CC(c2ccnc(-c3ccc4c(c3)Nc3nccc(N5CCOCC5)c3CO4)c2)C1]

Compound 61 was synthesized in a similar manner as Compound 25 using Intermediate 319 instead of Intermediate 218.

LC MS: confirms the MW (RT: 1.872, [M+H]⁺: 469.8, Method: 2).

¹H NMR (300 MHz, Chloroform-d) δ (ppm) 3.06 (t, J=4.4 Hz, 4H), 3.87 (t, J=4.5 Hz, 5H), 4.10-4.34 (m, 2H), 4.51 (t, J=9.7 Hz, 1H), 4.65 (t, J=8.7 Hz, 1H), 5.08 (s, 2H), 5.71 (dd, J=10.2, 2.0 Hz, 1H), 6.22 (dd, J=17.0, 10.2 Hz, 1H), 6.31-6.45 (m, 2H), 7.01 (d, J=8.4 Hz, 1H), 7.10-7.18 (m, 1H), 7.36 (dd, J=8.4, 2.0 Hz, 1H), 7.55 (s, 1H), 7.59-7.66 (m, 2H), 7.99 (d, J=5.7 Hz, 1H), 8.61 (d, J=5.1 Hz, 1H).

MP: 189.8° C. (Mettler Toledo MP50), uncorrected.

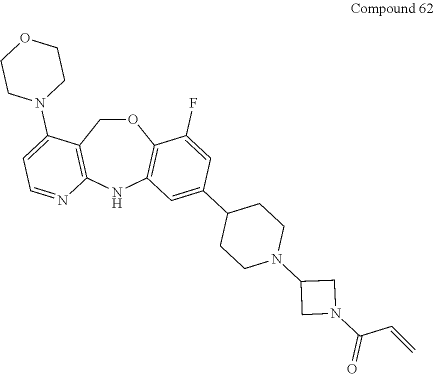 [SMILES: C=CC(=O)N1CC(N2CCC(c3cc(F)c4c(c3)Nc3nccc(N5CCOCC5)c3CO4)CC2)C1]

Compound 62 was synthesized in a similar manner as Compound 262 using Intermediate 324 instead of Intermediate 76.

LC MS: confirms the MW (RT: 1.484, [M+H]⁺: 493.8, Method: 2).

¹H NMR (300 MHz, Chloroform-d) δ (ppm) 1.64-1.79 (m, 2H), 1.85 (d, J=13.0 Hz, 2H), 1.91-2.02 (m, 2H), 2.40 (t, J=12.1 Hz, 1H), 2.86-2.99 (m, 2H), 3.03 (t, J=4.6 Hz, 4H), 3.13-3.27 (m, 1H), 3.87 (t, J=4.5 Hz, 4H), 3.96 (dd, J=10.4, 5.4 Hz, 1H), 4.05-4.16 (m, 2H), 4.24 (t, J=7.9 Hz, 1H), 5.12 (s, 2H), 5.66 (dd, J=10.1, 2.1 Hz, 1H), 6.19 (dd, J=17.0, 10.1 Hz, 1H), 6.33 (dd, J=17.0, 2.1 Hz, 1H), 6.38-6.44 (m, 2H), 6.50 (dd, J=11.3, 2.0 Hz, 1H), 6.81 (s, 1H), 8.01 (d, J=5.6 Hz, 1H).

MP: 209.9° C. (Mettler Toledo MP50), uncorrected.

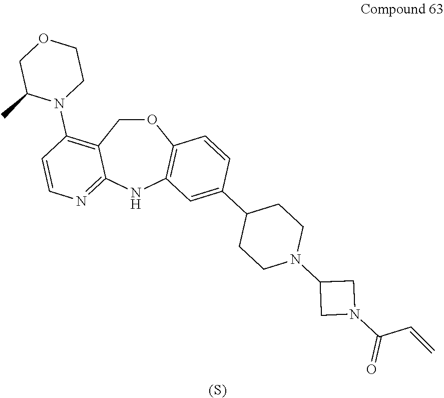 [SMILES: C=CC(=O)N1CC(N2CCC(c3ccc4c(c3)Nc3nccc(N5CCOC[C@@H]5C)c3CO4)CC2)C1 ; "(S)"]

Compound 63 was synthesized in a similar manner as Compound 3 using Intermediate 325 instead of Intermediate 10.

LC MS: confirms the MW (RT: 2.58, [M+H]⁺: 490.5, Method: 10).

¹H NMR: (500 MHz, DMSO-d₆, 22° C.): δ (ppm) 9.01 (s, 1H), 8.00 (d, J=5.7 Hz, 1H), 7.08 (d, J=1.9 Hz, 1H), 6.81 (d, J=8.2 Hz, 1H), 6.58 (dd, J=8.2, 1.9 Hz, 1H), 6.53 (d, J=5.7 Hz, 1H), 6.31 (dd, J=16.9, 10.2 Hz, 1H), 6.10 (dd, J=17.0, 2.2 Hz, 1H), 5.67 (dd, J=10.4, 2.2 Hz, 1H), 5.15 (d, J=13.6 Hz, 1H), 4.89 (d, J=13.6 Hz, 1H), 4.21-4.30 (m, 1H), 4.03 (br dd, J=9.1, 5.0 Hz, 1H), 3.94 (br dd, J=10.1, 6.9 Hz, 1H), 3.79-3.88 (m, 1H), 3.67-3.79 (m, 3H), 3.34-3.42 (m, 1H), 3.08-3.25 (m, 2H), 2.97 (br s, 1H), 2.82-2.94 (m, 2H), 2.63-2.70 (m, 2H), 2.31-2.41 (m, 1H), 1.80-1.99 (m, 2H), 1.73 (br d, J=13.2 Hz, 2H), 1.52-1.66 (m, 2H), 0.84 ppm (d, J=6.3 Hz, 3H)

OR: −39.55° (589 nm, e 0.1947 w/v %, DMF, 20° C.).

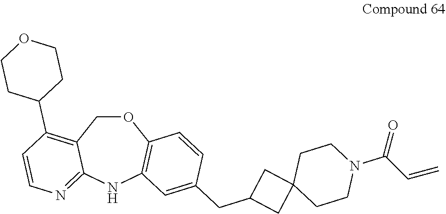 [SMILES: C=CC(=O)N1CCC2(CC1)CC(Cc1ccc3c(c1)Nc1nccc(C4CCOCC4)c1CO3)C2]

Compound 64 was synthesized in a similar manner as Compound 30 using Intermediate 327 instead of Intermediate 232.

LC MS: confirms the MW (RT: 2.53, [M+H]⁺: 474.3, Method: 4).

¹H NMR (500 MHz, Chloroform-d) δ (ppm) 1.52 (br s, 4H), 1.58-1.72 (m, 8H), 1.78-1.89 (m, 2H), 1.93-2.04 (m, 2H), 2.43-2.56 (m, 1H), 2.62 (d, J=7.5 Hz, 2H), 3.08 (tt, J=11.9, 3.6 Hz, 1H), 3.34-3.47 (m, 2H), 3.56 (td, J=11.8, 2.0 Hz, 4H), 4.09 (dd, J=11.3, 3.8 Hz, 2H), 5.08 (s, 2H), 5.64 (dd, J=10.7, 2.0 Hz, 1H), 6.23 (dd, J=16.8, 2.0 Hz, 1H), 6.50-6.61 (m, 3H), 6.66 (d, J=5.2 Hz, 1H), 6.84 (d, J=8.1 Hz, 1H), 6.87 (s, 1H), 8.06 (d, J=5.2 Hz, 1H).

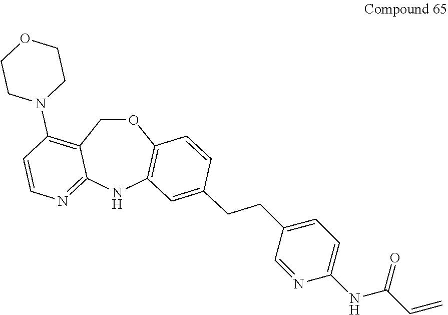 [SMILES: C=CC(=O)Nc1ccc(CCc2ccc3c(c2)Nc2nccc(N4CCOCC4)c2CO3)cn1]

Compound 65 was synthesized in a similar manner as Compound 49 starting with Intermediate 330 instead of Intermediate 289.

LC MS: confirms the MW (RT: 2.137, [M+H]⁺: 458.1, Method: 2).

¹H NMR (300 MHz, Chloroform-d) δ (ppm) 2.72-2.93 (m, 4H), 3.03 (t, J=4.6 Hz, 4H), 3.88 (t, J=4.5 Hz, 4H), 5.05 (s, 2H), 5.79 (dd, J=10.2, 1.5 Hz, 1H), 6.27 (dd, J=16.9, 10.2 Hz, 1H), 6.34-6.50 (m, 2H), 6.52-6.63 (m, 2H), 6.86 (d, J=8.1 Hz, 1H), 7.52 (dd, J=8.5, 2.3 Hz, 1H), 7.98 (d, J=5.7 Hz, 1H), 8.08 (d, J=2.2 Hz, 1H), 8.22 (d, J=8.5 Hz, 1H), 8.52 (s, 1H).

MP: 238.4° C. (Mettler Toledo MP50), uncorrected.

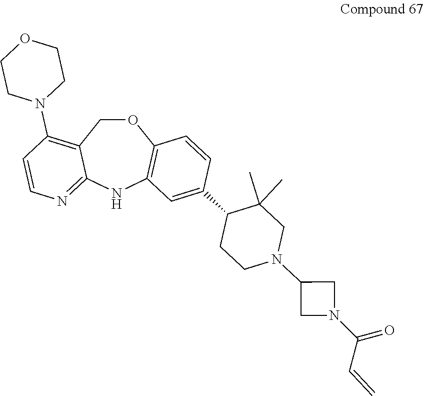 [SMILES: C=CC(=O)N1CC(N2CC[C@H](c3ccc4c(c3)Nc3nccc(N5CCOCC5)c3CO4)C(C)(C)C2)C1]

(*R), pure stereoisomer but absolute stereochemistry undetermined Compound 67 was synthesized in a similar manner as Compound 49 using Intermediate 333 instead of Intermediate 289.

LC MS: confirms the MW (RT: 1.707, [M+H]⁺: 504.299, Method: 3).

¹H NMR (300 MHz, Chloroform-d) δ (ppm) 0.75 (s, 3H), 0.90 (s, 3H), 1.57 (d, J=12.9 Hz, 1H), 1.73 (dd, J=10.9, 4.6 Hz, 1H), 1.87 (t, J=11.2 Hz, 1H), 2.08 (qd, J=12.5, 3.6 Hz, 1H), 2.24 (dd, J=12.9, 3.2 Hz, 1H), 2.36-2.48 (m, 1H), 2.86-2.99 (m, 1H), 3.03 (t, J=4.5 Hz, 4H), 3.13 (t, J=6.1 Hz, 1H), 3.87 (t, J=4.5 Hz, 4H), 3.91-4.28 (m, 4H), 5.06 (s, 2H), 5.60-5.74 (m, 1H), 6.21 (ddd, J=17.0, 10.1, 1.9 Hz, 1H), 6.29-6.46 (m, 2H), 6.54-6.65 (m, 2H), 6.77-6.92 (m, 2H), 7.98 (d, J=5.6 Hz, 1H).

MP: 151.4° C. (Mettler Toledo MP50), uncorrected.

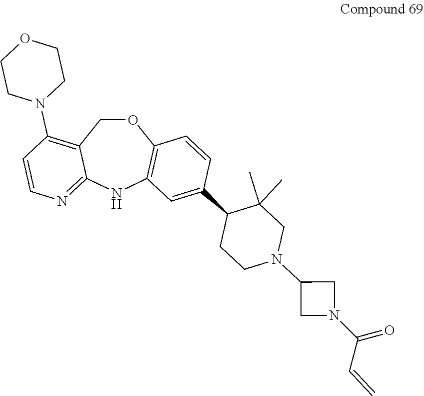 [SMILES: C=CC(=O)N1CC(N2CC[C@@H](c3ccc4c(c3)Nc3nccc(N5CCOCC5)c3CO4)C(C)(C)C2)C1]

(*S), pure stereoisomer but absolute stereochemistry undetermined Compound 69 was synthesized in a similar manner as Compound 49 using Intermediate 334 instead of Intermediate 289.

LC MS: confirms the MW (RT: 1.60, [M+H]⁺: 503.8, Method: 2).

¹H NMR (300 MHz, Chloroform-d) δ (ppm) 0.75 (s, 3H), 0.90 (s, 3H), 1.57 (d, J=12.9 Hz, 1H), 1.73 (dd, J=10.9, 4.6 Hz, 1H), 1.87 (t, J=11.2 Hz, 1H), 2.08 (qd, J=12.5, 3.6 Hz, 1H), 2.24 (dd, J=12.9, 3.2 Hz, 1H), 2.36-2.48 (m, 1H), 2.86-2.99 (m, 1H), 3.03 (t, J=4.5 Hz, 4H), 3.13 (t, J=6.1 Hz, 1H), 3.87 (t, J=4.5 Hz, 4H), 3.91-4.28 (m, 4H), 5.06 (s, 2H), 5.60-5.74 (m, 1H), 6.21 (ddd, J=17.0, 10.1, 1.9 Hz, 1H), 6.29-6.46 (m, 2H), 6.54-6.65 (m, 2H), 6.77-6.92 (m, 2H), 7.98 (d, J=5.6 Hz, 1H).

MP: 156.4° C. (Mettler Toledo MP50), uncorrected.

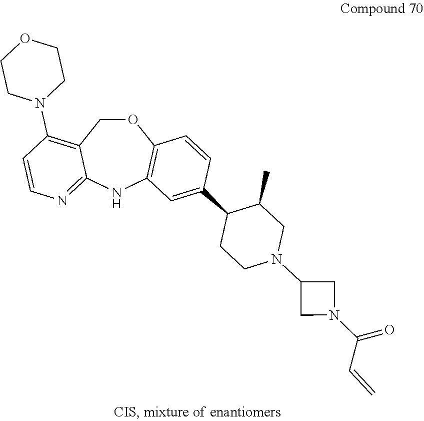 [SMILES: C=CC(=O)N1CC(N2CC[C@@H](c3ccc4c(c3)Nc3nccc(N5CCOCC5)c3CO4)[C@@H](C)C2)C1]

Compound 70 was synthesized in a similar manner as Compound 49 using Intermediate 336 instead of Intermediate 289.

LC MS: confirms the MW (RT: 1.60, [M+H]⁺: 490.6 Method: 3).

¹H NMR (300 MHz, Chloroform-d) δ (ppm) 0.79 (d, J=7.0 Hz, 3H), 1.62 (d, J=12.6 Hz, 1H), 1.84-2.08 (m, 3H), 2.09-2.21 (m, 1H), 2.63-2.76 (m, 2H), 2.92 (dd, J=15.7, 10.2 Hz, 1H), 3.02 (t, J=4.5 Hz, 4H), 3.07-3.20 (m, 1H), 3.86 (t, J=4.5 Hz, 4H), 3.89-4.03 (m, 1H), 4.03-4.14 (m, 2H), 4.14-4.25 (m, 1H), 5.04 (s, 2H), 5.65 (d, J=10.2 Hz, 1H), 6.19 (ddd, J=17.0, 10.0, 2.4 Hz, 1H), 6.30 (d, J=2.2 Hz, 1H), 6.36 (d, J=5.6 Hz, 1H), 6.51-6.63 (m, 2H), 6.87 (d, J=8.1 Hz, 1H), 7.11 (s, 1H), 7.97 (d, J=5.6 Hz, 1H).

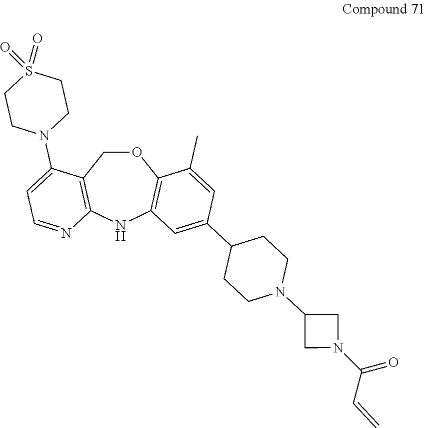 [SMILES: C=CC(=O)N1CC(N2CCC(c3cc(C)c4c(c3)Nc3nccc(N5CCS(=O)(=O)CC5)c3CO4)CC2)C1]

Compound 71 was synthesized in a similar manner as Compound 27 using Intermediate 338 instead of Intermediate 222.

LC MS: confirms the MW (RT: 1.533, [M+H]⁺: 538.3401, Method: 3).

¹H NMR (300 MHz, Chloroform-d) δ (ppm) 1.67-1.88 (m, 4H), 1.90-2.05 (m, 2H), 2.24 (s, 3H), 2.32-2.49 (m, 1H), 2.94 (t, J=13.8 Hz, 2H), 3.13-3.30 (m, 5H), 3.45-3.55 (m, 4H), 3.97 (dd, J=10.6, 5.4 Hz, 1H), 4.11 (dt, J=9.6, 6.0 Hz, 2H), 4.24 (t, J=7.9 Hz, 1H), 5.05 (s, 2H), 5.66 (dd, J=10.1, 2.1 Hz, 1H), 6.19 (dd, J=17.0, 10.1 Hz, 1H), 6.33 (dd, J=17.0, 2.1 Hz, 1H), 6.41 (d, J=5.6 Hz, 1H), 6.51 (d, J=2.0 Hz, 1H), 6.59 (s, 1H), 6.87 (s, 1H), 8.01 (d, J=5.6 Hz, 1H).

MP: 220° C. (Mettler Toledo MP 50), uncorrected.

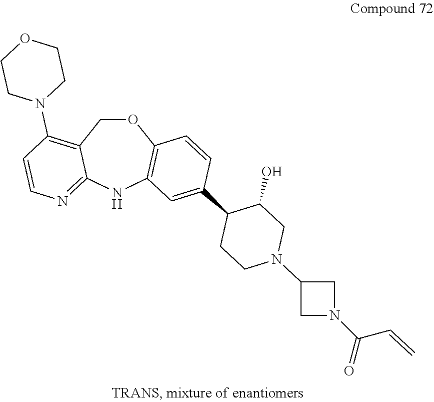 [SMILES: C=CC(=O)N1CC(N2CC[C@@H](c3ccc4c(c3)Nc3nccc(N5CCOCC5)c3CO4)[C@H](O)C2)C1]

Compound 72 was synthesized in a similar manner as Compound 173 using Intermediate 341 instead of Intermediate 65.

LC MS: confirms the MW (RT: 1.493, [M+H]⁺: 492.3877, Method: 3).

¹H NMR: (300 MHz, DMSO-d₆) δ (ppm) 8.91 (s, 1H), 7.97 (d, J=5.5 Hz, 1H), 7.06 (s, 1H), 6.79 (d, J=8.1 Hz, 1H), 6.57 (d, J=8.2 Hz, 1H), 6.43 (d, J=5.5 Hz, 1H), 6.32 (ddd, J=15.7, 10.3, 5.1 Hz, 1H), 6.10 (d, J=16.8 Hz, 1H), 5.66 (d, J=10.4 Hz, 1H), 4.96 (s, 2H), 4.56 (d, J=5.8 Hz, 1H), 4.29-4.21 (m, 1H), 4.10-3.89 (m, 2H), 3.79-3.69 (m, 5H), 3.58-3.48 (m, 1H), 3.23-3.14 (m, 1H), 2.95-2.87 (m, 5H), 2.85-2.72 (m, 1H), 2.24-2.14 (m, 1H), 1.88-1.76 (m, 1H), 1.76-1.49 (m, 3H).

MP: 181.3° C. (Mettler Toledo FP62) 10° C./min; uncorrected.

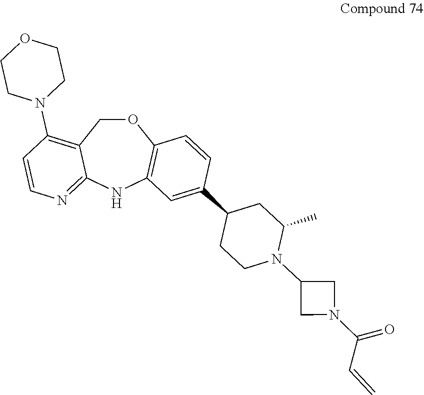 [SMILES: C=CC(=O)N1CC(N2CC[C@@H](c3ccc4c(c3)Nc3nccc(N5CCOCC5)c3CO4)C[C@@H]2C)C1]

(7*R, 11*S) pure isomer but absolute stereochemistry undetermined

Compound 74 was synthesized in a similar manner as Compound 49 using Intermediate 345 instead of Intermediate 289.

LC MS: confirms the MW (RT: 1.580, [M+H]⁺: 490.2793, Method: 3).

¹H NMR (300 MHz, Chloroform-d) δ (ppm) 0.97-1.09 (m, 3H), 1.63-1.76 (m, 2H), 1.76-2.04 (m, 2H), 2.44-2.82 (m, 3H), 2.97-3.07 (m, 4H), 3.18-3.35 (m, 1H), 3.53-3.70 (m, 1H), 3.80-3.93 (m, 4H), 3.92-4.05 (m, 1H), 4.03-4.17 (m, 2H), 4.17-4.30 (m, 1H), 5.05 (s, 2H), 5.61-5.71 (m, 1H), 6.20 (dd, J=17.0, 10.0 Hz, 1H), 6.30 (d, J=2.2 Hz, 1H), 6.37 (d, J=5.8 Hz, 1H), 6.58-6.70 (m, 2H), 6.75 (s, 1H), 6.88 (d, J=8.5 Hz, 1H), 7.99 (d, J=5.6 Hz, 1H).

MP: 236.8° C. (Mettler Toledo MP50), uncorrected.

OR: +11.79° (589 nm, c 0.135 w/v, chloroform, 23.0° C.).

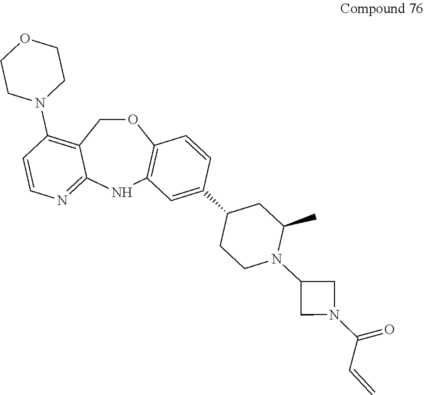 [SMILES: C=CC(=O)N1CC(N2CC[C@H](c3ccc4c(c3)Nc3nccc(N5CCOCC5)c3CO4)C[C@H]2C)C1]

(7*S, 11*R) pure isomer but absolute stereochemistry undetermined Compound 76 was synthesized in a similar manner as Compound 49 using Intermediate 346 instead of Intermediate 289.

LC MS: confirms the MW (RT: 1.573, [M+H]⁺: 490.2791, Method: 3).

¹H NMR (300 MHz, Chloroform-d) δ (ppm) 0.97-1.09 (m, 3H), 1.63-1.76 (m, 2H), 1.76-2.04 (m, 2H), 2.44-2.82 (m, 3H), 2.97-3.07 (m, 4H), 3.18-3.35 (m, 1H), 3.53-3.70 (m, 1H), 3.80-3.93 (m, 4H), 3.92-4.05 (m, 1H), 4.03-4.17 (m, 2H), 4.17-4.30 (m, 1H), 5.05 (s, 2H), 5.61-5.71 (m, 1H), 6.20 (dd, J=17.0, 10.0 Hz, 1H), 6.30 (d, J=2.2 Hz, 1H), 6.37 (d, J=5.8 Hz, 1H), 6.58-6.70 (m, 2H), 6.75 (s, 1H), 6.88 (d, J=8.5 Hz, 1H), 7.99 (d, J=5.6 Hz, 1H).

MP: 233.4° C. (Mettler Toledo MP50), uncorrected.

OR: −9.8477° (589 nm, c 0.131 w/v, Chloroform, 23.0° C.).

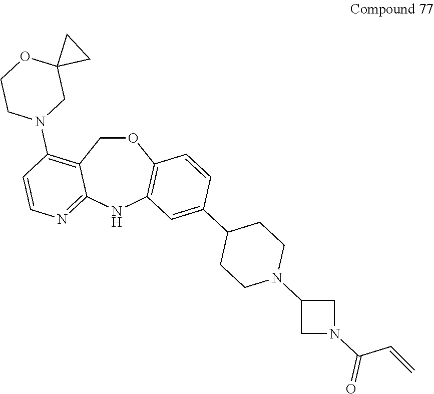 [SMILES: C=CC(=O)N1CC(N2CCC(c3ccc4c(c3)Nc3nccc(N5CCOC6(CC6)C5)c3CO4)CC2)C1]

Compound 77 was synthesized in a similar manner as Compound 3 using Intermediate 347 instead of Intermediate 10.

LC MS: confirms the MW (RT: 2.81, [M+H]⁺: 502.4, Method: 1).

¹H NMR: (500 MHz, DMSO-d₆, 22° C.): δ (ppm) 8.94 (s, 1H), 7.97 (d, J=5.7 Hz, 1H), 7.07 (d, J=1.9 Hz, 1H), 6.81 (d, J=8.2 Hz, 1H), 6.58 (dd, J=8.4, 2.0 Hz, 1H), 6.44 (d, J=5.7 Hz, 1H), 6.31 (dd, J=17.0, 10.4 Hz, 1H), 6.10 (dd, J=17.0, 2.2 Hz, 1H), 5.76 (s, 1H), 5.65-5.69 (m, 1H), 4.98 (s, 2H), 4.24 (t, J=7.9 Hz, 1H), 4.03 (dd, J=9.0, 4.9 Hz, 1H), 3.94 (dd, J=10.1, 7.3 Hz, 1H), 3.71-3.83 (m, 3H), 3.10-3.16 (m, 1H), 2.95-3.01 (m, 2H), 2.84-2.93 (m, 4H), 2.32-2.40 (m, 2H), 1.84-1.93 (m, 2H), 1.67-1.77 (m, 2H), 1.51-1.63 (m, 2H), 0.70-0.78 (m, 2H), 0.56-0.65 ppm (m, 2H)

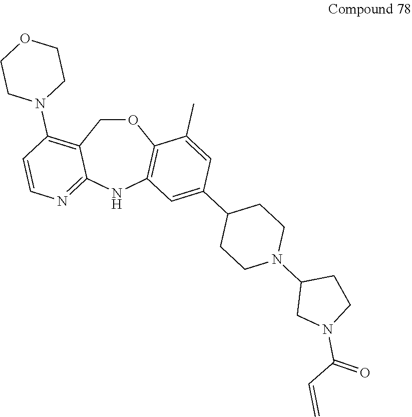 [SMILES: C=CC(=O)N1CCC(N2CCC(c3cc(C)c4c(c3)Nc3nccc(N5CCOCC5)c3CO4)CC2)C1]

Compound 78 was synthesized in a similar manner as Compound 3 using Intermediate 348 instead of Intermediate 10.

LC MS: confirms the MW (RT: 2.69, [M+H]⁺: 504.4, Method: 1).

¹H NMR: (500 MHz, DMSO-d₆, 25° C.): δ (ppm) 8.84 (d, J=1.9 Hz, 1H), 7.96 (d, J=5.4 Hz, 1H), 6.90 (s, 1H), 6.50-6.65 (m, 2H), 6.43 (d, J=5.7 Hz, 1H), 6.10-6.16 (m, 1H), 5.65 (ddd, J=10.1, 7.6, 2.5 Hz, 1H), 4.98 (s, 2H), 3.73-3.92 (m, 5H), 3.44-3.66 (m, 1H), 3.22-3.29 (m, 1H), 3.00-3.09 (m, 1H), 2.73-2.96 (m, 6H), 2.27-2.36 (m, 1H), 2.16 (s, 3H), 2.02-2.11 (m, 2H), 1.52-1.85 ppm (m, 5H)

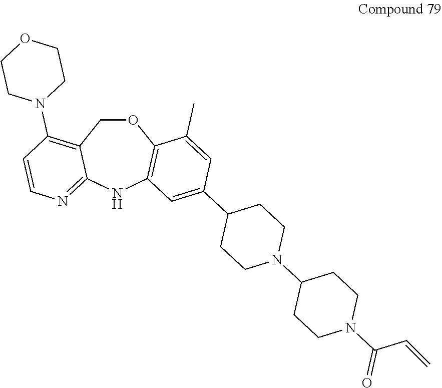 [SMILES: C=CC(=O)N1CCC(N2CCC(c3cc(C)c4c(c3)Nc3nccc(N5CCOCC5)c3CO4)CC2)CC1]

Compound 79 was synthesized in a similar manner as Compound 3 using Intermediate 349 instead of Intermediate 10.

LC MS: confirms the MW (RT: 2.45, [M+H]⁺: 518.5, Method: 1).

¹H NMR: (500 MHz, DMSO-d₆, 26° C.): δ (ppm) 8.83 (s, 1H), 7.96 (d, J=5.4 Hz, 1H), 6.89 (s, 1H), 6.80 (dd, J=16.7, 10.4 Hz, 1H), 6.50 (s, 1H), 6.43 (d, J=5.4 Hz, 1H), 6.07 (dd, J=16.6, 2.4 Hz, 1H), 5.65 (dd, J=10.4, 2.2 Hz, 1H), 4.97 (s, 2H), 4.45 (br d, J=10.4 Hz, 1H), 4.08 (br d, J=11.0 Hz, 1H), 3.75 (br s, 4H), 2.97-3.09 (m, 1H), 2.92 (br s, 2H), 2.86 (br s, 4H), 2.58-2.65 (m, 1H), 2.24 (br s, 3H), 2.15 (s, 3H), 1.79 (br d, J=11.7 Hz, 2H), 1.70 (br d, J=12.3 Hz, 2H), 1.43-1.61 (m, 2H), 1.32 (br s, 2H), 1.24 ppm (br s, 1H)

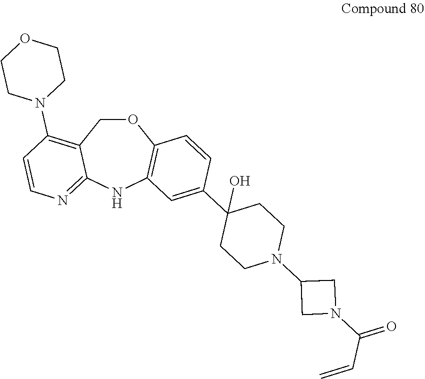 [SMILES: C=CC(=O)N1CC(N2CCC(O)(c3ccc4c(c3)Nc3nccc(N5CCOCC5)c3CO4)CC2)C1]

DCM (10 mL) was added to a mixture of Intermediate 354 (153 mg, 0.35 mmol), Acryloyl Chloride (3.03 mL, 0.37 mmol) and Triethylamine (0.15 mL, 1.05 mmol) in DMF (1 mL) and the reaction mixture was stirred at 0° C. for 90 minutes. DCM and aq Na₂CO₃ 1M were added and the organics were separated, the organic layer was dried over MgSO₄, filtered, and concentrated to dryness. The product was purified by reverse phase chromatography (Phenomenex Gemini C18 30×100 mm 5 μm; gradient from 81% H₂O−19% ACN−MeOH) to 45% H₂O−55% ACN-MeOH-[25 mM NH₄HCO₃]). The product was triturated in diethyl ether to give Compound 80 (70 mg, 40% yield) as a solid.

LC MS: confirms the MW (RT: 1.464, [M+H]⁺: 492, Method: 2).

¹H NMR: (300 MHz, DMSO-d₆) δ (ppm) 8.91 (s, 1H), 7.97 (d, J=5.4 Hz, 1H), 7.34 (s, 1H), 6.82 (s, 2H), 6.43 (d, J=5.5 Hz, 1H), 6.31 (dd, J=17.0, 10.3 Hz, 1H), 6.09 (d, J=15.8 Hz, 1H), 5.66 (d, J=10.5 Hz, 1H), 4.97 (s, 2H), 4.71 (s, 1H), 4.24 (t, J=7.8 Hz, 1H), 4.07-3.90 (m, 2H), 3.81-3.69 (m, 5H), 3.21-3.10 (m, 1H), 2.95-2.87 (m, 4H), 2.58 (t, J=10.6 Hz, 2H), 2.28 (t, J=10.6 Hz, 2H), 1.85 (t, J=11.2 Hz, 2H), 1.60 (d, J=12.5 Hz, 2H).

MP: 137.0° C. (Mettler Toledo FP62), 10° C./min, uncorrected.

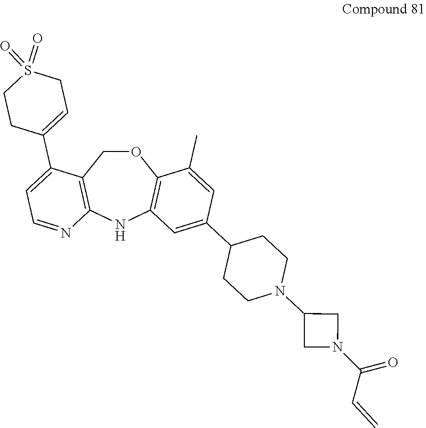 [SMILES: C=CC(=O)N1CC(N2CCC(c3cc(C)c4c(c3)Nc3nccc(C5=CCS(=O)(=O)CC5)c3CO4)CC2)C1]

Compound 81 was synthesized in a similar manner as Compound 49 using Intermediate 358 instead of Intermediate 289.

LC MS: confirms the MW (R T: 1.740, [M+H]⁺: 535.2388, Method: 3)

¹H NMR (300 MHz, Chloroform-d) δ (ppm) 1.61-1.79 (m, 2H), 1.79-1.89 (m, 2H), 1.89-2.02 (m, 2H), 2.22 (s, 3H), 2.33-2.49 (m, 1H), 2.86-3.04 (m, 4H), 3.14-3.31 (m, 3H), 3.74-3.86 (m, 2H), 3.96 (dd, J=10.6, 5.4 Hz, 1H), 4.05-4.18 (m, 2H), 4.23 (t, J=7.9 Hz, 1H), 4.95 (s, 2H), 5.54-5.60 (m, 1H), 5.60-5.71 (m, 1H), 6.11-6.24 (m, 1H), 6.26-6.36 (m, 1H), 6.49 (d, J=5.1 Hz, 1H), 6.52-6.62 (m, 2H), 7.28 (s, 1H), 8.02 (d, J=5.1 Hz, 1H).

MP: >300° C. (Mettler Toledo MP50), uncorrected.

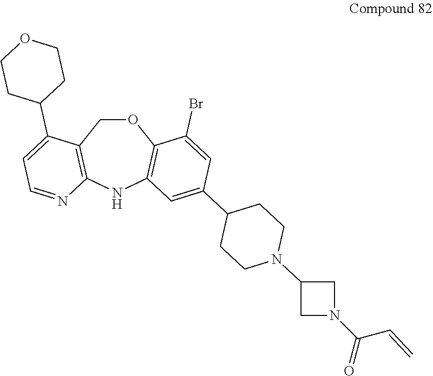 [SMILES: C=CC(=O)N1CC(N2CCC(c3cc(Br)c4c(c3)Nc3nccc(C5CCOCC5)c3CO4)CC2)C1]

Compound 82 was synthesized in a similar manner as Compound 173 using Intermediate 365 instead of Intermediate 65.

LC MS: confirms the MW (RT: 2.067, [M+H]⁺: 553.1818, Method: 3).

¹H NMR: (300 MHz, Chloroform-d) δ (ppm) 8.08 (d, J=5.3 Hz, 1H), 6.94 (s, 2H), 6.70 (d, J=5.3 Hz, 1H), 6.60 (s, 1H), 6.33 (d, J=16.9 Hz, 1H), 6.19 (dd, J=17.0, 10.1 Hz, 1H), 5.66 (d, J=10.2 Hz, 1H), 5.15 (s, 2H), 4.24 (t, J=7.9 Hz, 1H), 4.15-4.06 (m, 4H), 3.96 (dd, J=10.3, 5.4 Hz, 1H), 3.57 (t, J=11.5 Hz, 2H), 3.24-3.14 (m, 1H), 3.13-3.04 (m, 1H), 3.03-2.85 (m, 2H), 2.47-2.34 (m, 1H), 1.98-1.75 (m, 6H), 1.77-1.64 (m, 4H).

MP: 151.5° C. (Mettler Toledo FP62), 10° C./min; uncorrected.

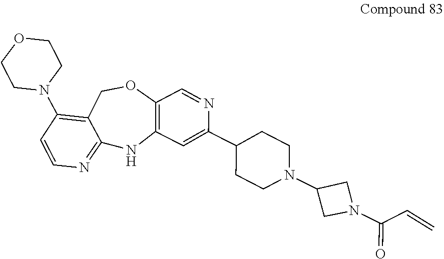 [SMILES: C=CC(=O)N1CC(N2CCC(c3cc4c(cn3)OCc3c(N5CCOCC5)ccnc3N4)CC2)C1]

To a solution of Intermediate 38 (614 mg, 1.454 mmol) in DCM (12 mL), Et₃N (3 mL, 21.8 mmol, 15 eq.) was added. The mixture was cooled in a ice bath and acryloyl chloride (118 μL, 1.454 mmol, 1 eq.) in DCM (6 mL) was added dropwise and the reaction mixture was stirred at room temperature for 2 h. The reaction was quenched by addition of saturated aqueous NaHCO₃ and the mixture was extracted with DCM. The organic layer was dried over MgSO₄ and concentrated under vacuum. The residue was purified by column flash chromatography on silica gel (DCM/DCM:MeOH (9:1) gradient) followed by reverse phase chromatography (Phenomenex Gemini C18 30×100 mm 5 μm; gradient from 81% [65 mM NH₄OAc+ACN (90:10)]−19% [ACN:MeOH (1:1)] to 45% [65 mM NH₄OAc+ACN (90:10)]−55% [ACN:MeOH (1:1)]). The solid obtained was triturated in ACN. The white solid was filtered to afford Compound 83 (135 mg, yield: 19%) as a white solid.

LCMS: confirms the MW (RT: 1.37, [M+H]⁺ 477, Method: 3).

MP: 238.4° C. (Mettler Toledo MP50).

¹H NMR (300 MHz, Chloroform-d) d (ppm) 1.81 (d, J=3.9 Hz, 1H), 1.99 (ddd, J=13.3, 9.6, 3.8 Hz, 5H), 2.62 (ddd, J=12.0, 8.4, 3.7 Hz, 1H), 2.95 (t, J=13.6 Hz, 2H), 3.01-3.09 (m, 4H), 3.21 (tt, J=7.2, 5.5 Hz, 1H), 3.85-3.91 (m, 4H), 3.97 (dd, J=10.5, 5.5 Hz, 1H), 4.11 (dt, J=8.5, 5.9 Hz, 1H), 4.23 (t, J=7.9 Hz, 1H), 5.04 (s, 21H), 5.64 (d, J=2.1 Hz, 1H), 5.68 (d, J=2.1 Hz, 1H), 6.19 (dd, J=17.0, 10.1 Hz, 1H), 6.33 (dd, J=17.0, 2.1 Hz, 1H), 6.47 (d, J=5.6 Hz, 1H), 6.54 (s, 1H), 7.03 (s, 1H), 8.04 (d, J=5.6 Hz, 1H), 8.11 (s, 1H).

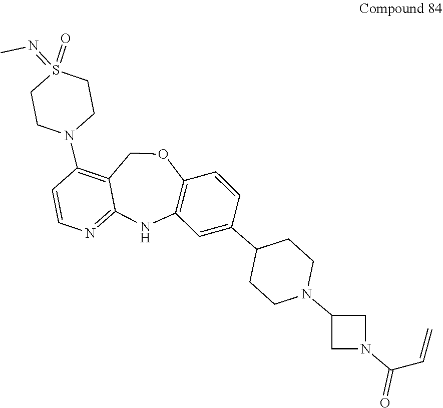 [SMILES: C=CC(=O)N1CC(N2CCC(c3ccc4c(c3)Nc3nccc(N5CCS(=O)(=NC)CC5)c3CO4)CC2)C1]

Compound 84 was synthesized in a similar manner as Compound 3 using Intermediate 366 instead of Intermediate 10.

LC MS: confirms the MW (RT: 2.13, [M+H]⁺: 537.4, Method: 1).

¹H NMR: (400 MHz, DMSO-d₆, 24° C.): δ (ppm) 8.99 (s, 1H), 7.97 (d, J=5.6 Hz, 1H), 7.08 (d, J=2.0 Hz, 1H), 6.82 (s, 1H), 6.80 (s, 1H), 6.59 (dd, J=8.3, 1.8 Hz, 1H), 6.51 (d, J=5.6 Hz, 1H), 6.31 (dd, J=16.9, 10.4 Hz, 1H), 6.10 (dd, J=17.2, 2.0 Hz, 1H), 5.64-5.70 (m, 1H), 5.00 (s, 2H), 4.24 (br t, J=7.8 Hz, 2H), 4.01-4.06 (m, 2H), 3.94 (br dd, J=10.1, 7.6 Hz, 2H), 3.74 (br dd, J=10.1, 5.1 Hz, 2H), 3.27-3.31 (m, 3H), 3.10-3.16 (m, 1H), 2.84-2.93 (m, 3H), 2.60-2.73 (m, 1H), 1.99 (s, 1H), 1.94 (br d, J=2.5 Hz, 2H), 1.71-1.80 (m, 2H), 1.57 (dd, J=12.1, 3.5 Hz, 2H)

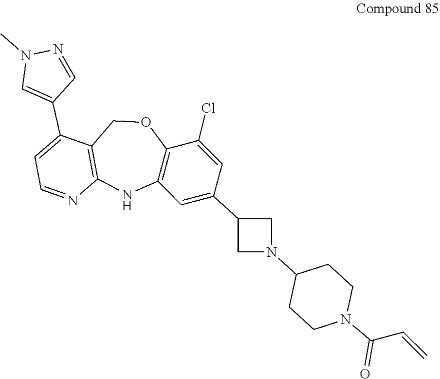 [SMILES: C=CC(=O)N1CCC(N2CC(c3cc(Cl)c4c(c3)Nc3nccc(-c5cnn(C)c5)c3CO4)C2)CC1]

Compound 85 was synthesised in a similar manner as Compound 164 using Intermediate 369 instead of Intermediate 50.

LC MS: confirms the MW (RT: 2.034, [M+H]⁺: 505.1, Method: 2).

¹H NMR: (300 MHz, DMSO-d₆) δ (ppm) 9.42 (s, 1H), 8.08 (d, J=5.0 Hz, 1H), 7.99 (s, 1H), 7.65 (s, 1H), 7.25 (s, 1H), 6.86 (s, 1H), 6.84-6.76 (m, 2H), 6.07 (d, J=16.7 Hz, 1H), 5.64 (d, J=12.3 Hz, 1H), 5.13 (s, 2H), 3.92 (s, 3H), 4.05-3.76 (m, 2H), 3.56 (t, J=6.7 Hz, 2H), 3.49-3.36 (m, 1H), 3.29-3.17 (m, 1H), 3.12-2.99 (m, 3H), 2.39-2.29 (m, 1H), 1.70-1.56 (m, 2H), 1.24-1.05 (m, 2H).

MP: 152.9° C. (Mettler Toledo FP62), 10° C./min, uncorrected.

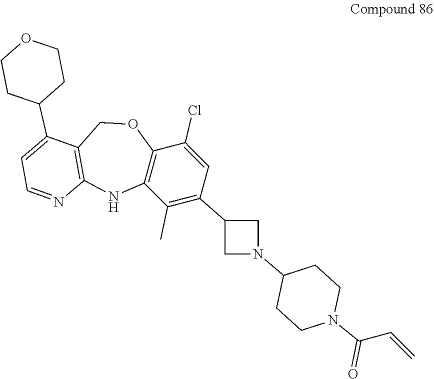 [SMILES: C=CC(=O)N1CCC(N2CC(c3cc(Cl)c4c(c3C)Nc3nccc(C5CCOCC5)c3CO4)C2)CC1]

Compound 86 was synthesized in a similar manner as Compound 173 using Intermediate 374 instead of Intermediate 65.

LC MS: confirms the MW (RT: 2.077, [M+H]⁺: 523.1, Method: 2).

¹H NMR (300 MHz, DMSO-d₆) δ (ppm) 8.07 (d, J=5.2 Hz, 1H), 7.65 (s, 1H), 6.84 (s, 1H), 6.80-6.74 (m, 2H), 6.06 (d, J=16.7 Hz, 1H), 5.64 (d, J=12.6 Hz, 1H), 5.20 (s, 2H), 4.09-3.76 (m, 4H), 3.71-3.62 (m, 3H), 3.51 (t, J=10.7 Hz, 2H), 3.26-2.87 (m, 5H), 3.30-2.19 (m, 1H), 2.14 (s, 3H), 1.72-1.52 (m, 6H), 1.20-1.02 (m, 2H).

MP: 201.7° C. (Mettler Toledo FP 62), 10° C./min uncorrected.

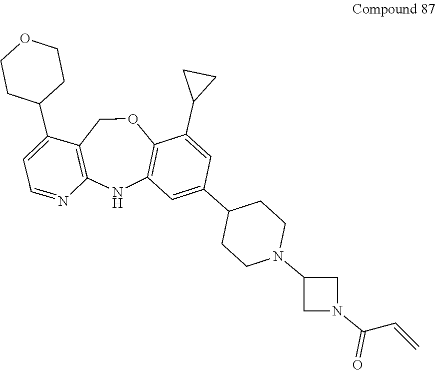 [SMILES: C=CC(=O)N1CC(N2CCC(c3cc4c(c(C5CC5)c3)OCc3c(C5CCOCC5)ccnc3N4)CC2)C1]

Compound 87 was synthesized in a similar manner as Compound 173 using Intermediate 377 instead of Intermediate 65.

LC MS: confirms the MW (R T: 1.947, [M+H]⁺: 515.3237, Method: 3).

¹H NMR: (300 MHz, Chloroform-d) δ (ppm) 8.05 (d, J=5.3 Hz, 1H), 6.90 (br s, 1H), 6.64 (d, J=5.3 Hz, 1H), 6.47 (s, 1H), 6.39-6.27 (m, 1H), 6.23-6.14 (m, 2H), 5.73-5.61 (m, 1H), 5.13 (s, 2H), 4.24 (t, J=7.9 Hz, 1H), 4.15-4.06 (m, 4H), 3.97 (dd, J=10.3, 5.4 Hz, 1H), 3.57 (t, J=11.4 Hz, 2H), 3.23-3.15 (m, 1H), 3.12-3.01 (m, 1H), 3.01-2.84 (m, 2H), 2.44-2.33 (m, 1H), 2.32-2.19 (m, 1H), 1.95 (t, J=11.7 Hz, 2H), 1.81 (t, J=12.1 Hz, 6H), 1.75-1.59 (m, 2H), 1.01-0.89 (m, 2H), 0.64-0.61 (m, 2H).

MP: 214.5° C. (Mettler Toledo FP62), 10° C./min; uncorrected.

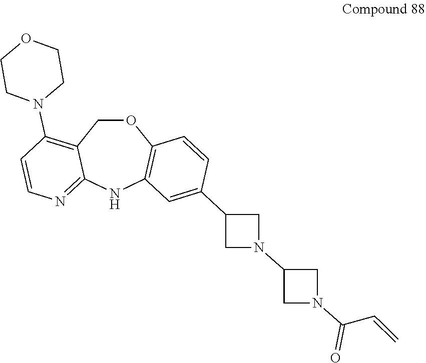 [SMILES: C=CC(=O)N1CC(N2CC(c3ccc4c(c3)Nc3nccc(N5CCOCC5)c3CO4)C2)C1]

Compound 88 was synthesized in a similar manner as Compound 3 using Intermediate 381 instead of Intermediate 10.

LC MS: confirms the MW (RT: 2.34, [M+H]⁺: 448.2, Method: 1).

¹H NMR: (500 MHz, DMSO-d₆, 30° C.): δ (ppm) 8.89 (s, 1H), 7.91 (d, J=5.7 Hz, 1H), 7.15 (d, J=2.2 Hz, 1H), 6.76 (d, J=8.2 Hz, 1H), 6.60 (dd, J=8.2, 1.9 Hz, 1H), 6.37 (d, J=5.7 Hz, 1H), 6.24 (dd, J=17.0, 10.1 Hz, 1H), 6.02 (dd, J=17.0, 2.2 Hz, 1H), 5.59 (dd, J=10.4, 2.2 Hz, 1H), 4.87-4.97 (m, 2H), 4.14 (br t, J=8.0 Hz, 1H), 3.80-3.99 (m, 2H), 3.61-3.75 (m, 5H), 3.49-3.57 (m, 2H), 3.39-3.47 (m, 2H), 3.04 (q, J=7.0 Hz, 2H), 2.80-2.88 ppm (m, 4H)

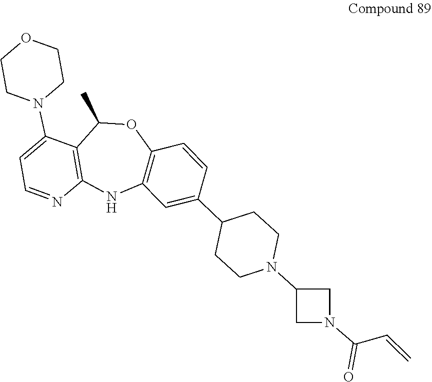 [SMILES: C=CC(=O)N1CC(N2CCC(c3ccc4c(c3)Nc3nccc(N5CCOCC5)c3[C@@H](C)O4)CC2)C1]

(*R), pure stereoisomer but absolute stereochemistry undetermined Compound 89 was synthesized in a similar manner as Compound 27 using Intermediate 387 instead of Intermediate 222.

LC MS: confirms the MW (RT: 1.669, [M+H]⁺: 490.1, Method: 2).

¹H NMR (300 MHz, Chloroform-d) δ (ppm) 1.41 (d, J=6.9 Hz, 3H), 1.64-1.82 (m, 2H), 1.83-2.07 (m, 4H), 2.45 (t, J=12.2 Hz, 1H), 2.78-3.02 (m, 4H), 3.02-3.13 (m, 2H), 3.20 (p, J=6.3 Hz, 1H), 3.78-3.92 (m, 4H), 3.91-4.03 (m, 1H), 4.08-4.18 (m, 2H), 4.24 (t, J=7.9 Hz, 1H), 5.66 (dd, J=10.1, 2.1 Hz, 1H), 5.84 (q, J=6.9 Hz, 1H), 6.20 (dd, J=17.0, 10.1 Hz, 1H), 6.33 (dd, J=17.0, 2.1 Hz, 1H), 6.45 (d, J=5.5 Hz, 1H), 6.59 (d, J=1.9 Hz, 1H), 6.63-6.68 (m, 1H), 6.87 (d, J=8.2 Hz, 1H), 6.94 (s, 1H), 8.00 (d, J=5.5 Hz, 1H).

MP: 241.7° C. (Mettler Toledo MP50).

OR: +47° (589 nm, c 0.0667 w/v, MeOH, 23° C.).

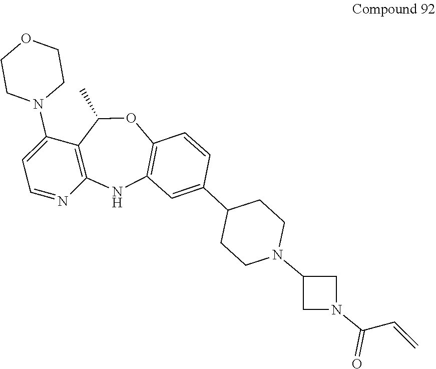 [SMILES: C=CC(=O)N1CC(N2CCC(c3ccc4c(c3)Nc3nccc(N5CCOCC5)c3[C@H](C)O4)CC2)C1]

(*S), pure stereoisomer but absolute stereochemistry undetermined Compound 92 was synthesized in a similar manner as Compound 27 using Intermediate 388 instead of Intermediate 222.

LC MS: confirms the MW (RT: 1.692, [M+H]⁺: 490.1, Method: 2).

¹H NMR (300 MHz, Chloroform-d) δ (ppm) 1.41 (d, J=6.9 Hz, 3H), 1.64-1.82 (m, 2H), 1.83-2.07 (m, 4H), 2.45 (t, J=12.2 Hz, 1H), 2.78-3.02 (m, 4H), 3.02-3.13 (m, 2H), 3.20 (p, J=6.3 Hz, 1H), 3.78-3.92 (m, 4H), 3.91-4.03 (m, 1H), 4.08-4.18 (m, 2H), 4.24 (t, J=7.9 Hz, 1H), 5.66 (dd, J=10.1, 2.1 Hz, 1H), 5.84 (q, J=6.9 Hz, 1H), 6.20 (dd, J=17.0, 10.1 Hz, 1H), 6.33 (dd, J=17.0, 2.1 Hz, 1H), 6.45 (d, J=5.5 Hz, 1H), 6.59 (d, J=1.9 Hz, 1H), 6.63-6.68 (m, 1H), 6.87 (d, J=8.2 Hz, 1H), 6.94 (s, 1H), 8.00 (d, J=5.5 Hz, 1H).

OR: −38.34° (589 nm, c 0.0847 w/v, MeOH, 23° C.).

MP: 238.3° C. (Mettler Toledo MP50), uncorrected.

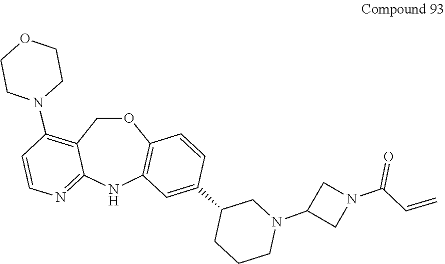 [SMILES: C=CC(=O)N1CC(N2CCC[C@H](c3ccc4c(c3)Nc3nccc(N5CCOCC5)c3CO4)C2)C1]

(*R), pure stereoisomer but absolute stereochemistry undetermined Compound 93 was synthesized in a similar manner as Compound 3 using Intermediate 391 instead of Intermediate 10.

LC MS: confirms the MW (RT: 2.57, [M+H]⁺: 476.3, Method: 1).

¹H NMR: (500 MHz, DMSO-d₆, 22° C.): δ (ppm) 8.91 (br d, J=4.4 Hz, 1H), 7.97 (d, J=5.7 Hz, 1H), 7.11 (s, 1H), 6.81 (d, J=8.2 Hz, 1H), 6.60 (br d, J=8.2 Hz, 1H), 6.44 (d, J=5.4 Hz, 1H), 6.29 (dt, J=17.0, 11.0 Hz, 1H), 6.09 (br d, J=17.0 Hz, 1H), 5.62-5.69 (m, 1H), 4.96 (s, 2H), 4.07-4.28 (m, 1H), 3.99-4.07 (m, 1H), 3.92 (ddd, J=13.2, 10.1, 7.6 Hz, 1H), 3.68-3.80 (m, 5H), 3.09-3.19 (m, 1H), 2.87-2.93 (m, 4H), 2.71-2.86 (m, 2H), 2.55-2.62 (m, 1H), 1.70-1.86 (m, 4H), 1.56 (q, J=11.6 Hz, 1H), 1.37 ppm (q, J=12.5 Hz, 1H)

OR: +80.23° (589 nm, c 0.258 w/v %, DMF, 20° C.).

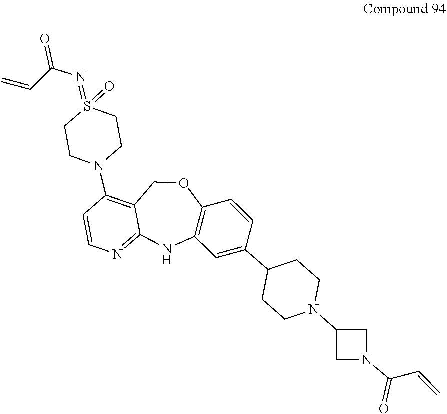 [SMILES: C=CC(=O)N=S1(=O)CCN(c2ccnc3c2COc2ccc(C4CCN(C5CN(C(=O)C=C)C5)CC4)cc2N3)CC1]

Compound 94 was synthesized in a similar manner as Compound 3 using Intermediate 395 instead of Intermediate 10.

LC MS: confirms the MW (RT: 2.37, [M+H]⁺: 577.5, Method: 1).

¹H NMR: (500 MHz, DMSO-d₆, 22° C.): δ (ppm) 9.04 (s, 1H), 7.97 (d, J=5.4 Hz, 1H), 7.08 (d, J=1.9 Hz, 1H), 6.82 (d, J=8.2 Hz, 1H), 6.60 (dd, J=8.4, 2.0 Hz, 1H), 6.53 (d, J=5.7 Hz, 1H), 6.15-6.34 (m, 3H), 6.07-6.13 (m, 1H), 5.74-5.78 (m, 1H), 5.65-5.69 (m, 1H), 5.03 (s, 2H), 4.20-4.30 (m, 1H), 4.04 (br d, J=8.5 Hz, 1H), 3.88-3.98 (m, 1H), 3.83 (br s, 2H), 3.69-3.78 (m, 3H), 3.40-3.50 (m, 2H), 3.10-3.17 (m, 1H), 2.84-2.94 (m, 2H), 2.34-2.39 (m, 1H), 1.84-1.93 (m, 2H), 1.70-1.78 (m, 2H), 1.52-1.63 ppm (m, 2H)

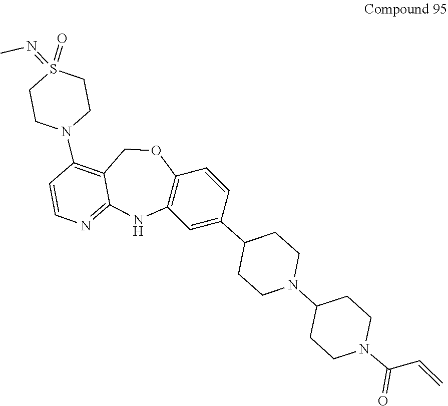 [SMILES: C=CC(=O)N1CCC(N2CCC(c3ccc4c(c3)Nc3nccc(N5CCS(=O)(=NC)CC5)c3CO4)CC2)CC1]

Compound 95 was synthesized in a similar manner as Compound 3 using Intermediate 397 instead of Intermediate 10.

LC MS: confirms the MW (RT: 1.93, [M+H]⁺: 565.5, Method: 1).

¹H NMR: (500 MHz, DMSO-d₆, 22° C.): δ (ppm) 8.98 (s, 1H), 7.97 (d, J=5.4 Hz, 1H), 7.08 (d, J=1.6 Hz, 1H), 6.77-6.87 (m, 2H), 6.58 (dd, J=8.4, 1.7 Hz, 1H), 6.52 (d, J=5.4 Hz, 1H), 6.08 (dd, J=16.7, 2.5 Hz, 1H), 5.66 (dd, J=10.6, 2.4 Hz, 1H), 5.00 (s, 2H), 4.46 (br d, J=12.0 Hz, 1H), 4.09 (br d, J=12.6 Hz, 1H), 3.27-3.31 (m, 6H), 2.94 (br s, 1H), 2.63-2.69 (m, 4H), 2.35 (br d, J=18.0 Hz, 2H), 1.80 (br d, J=10.7 Hz, 2H), 1.72 (br d, J=11.3 Hz, 2H), 1.46-1.60 (m, 2H), 1.27-1.40 ppm (m, 2H)

MP: 235.87° C./−190.82 J/g (DSC: 25° C. to 350° C./10° C. min/40 μl Al).

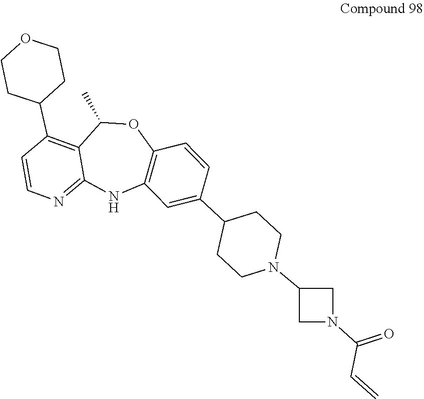 [SMILES: C=CC(=O)N1CC(N2CCC(c3ccc4c(c3)Nc3nccc(C5CCOCC5)c3[C@H](C)O4)CC2)C1]

(*S), pure stereoisomer but absolute stereochemistry undetermined Compound 98 was synthesized in a similar manner as Compound 49 using Intermediate 402 instead of Intermediate 289.

LC MS: confirms the MW (RT: 1.799, [M+H]⁺: 489.2854, Method: 3).

¹H NMR (300 MHz, Chloroform-d) δ (ppm) 1.37 (d, J=6.9 Hz, 3H), 1.57-1.66 (m, 2H), 1.69-2.04 (m, 8H), 2.46 (t, J=11.9 Hz, 1H), 2.83-3.12 (m, 3H), 3.21 (p, J=6.4 Hz, 1H), 3.55 (ddd, J=14.7, 10.6, 3.5 Hz, 2H), 3.98 (dd, J=10.5, 5.5 Hz, 1H), 4.05-4.19 (m, 4H), 4.24 (t, J=7.9 Hz, 1H), 5.66 (dd, J=10.1, 2.1 Hz, 1H), 5.77 (q, J=6.9 Hz, 1H), 6.20 (dd, J=17.0, 10.1 Hz, 1H), 6.33 (dd, J=17.0, 2.1 Hz, 1H), 6.61 (d, J=1.8 Hz, 1H), 6.66 (t, J=7.1 Hz, 2H), 6.89 (d, J=8.2 Hz, 1H), 7.06 (s, 1H), 8.04 (d, J=5.2 Hz, 1H).

MP: 255.0° C. (Mettler Toledo MP50), uncorrected.

OR: −8.47° (589 nm, c 0.1247 w/v, CHCl₃).

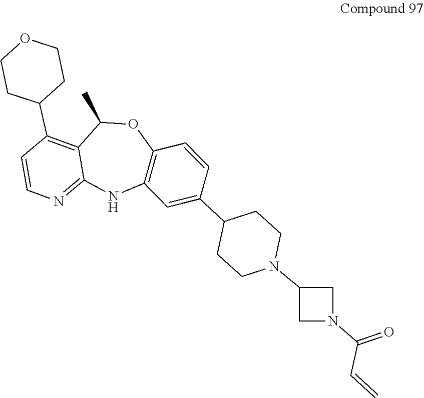 [SMILES: C=CC(=O)N1CC(N2CCC(c3ccc4c(c3)Nc3nccc(C5CCOCC5)c3[C@@H](C)O4)CC2)C1]

(*R), pure stereoisomer but absolute stereochemistry undetermined Compound 97 was synthesized in a similar manner as Compound 49 using Intermediate 401 instead of Intermediate 289.

LC MS: confirms the MW (RT: 1.653, [M+H]⁺: 489.3046, Method: 3).

¹H NMR (300 MHz, Chloroform-d) δ (ppm) 1.37 (d, J=6.9 Hz, 3H), 1.57-1.66 (m, 2H), 1.69-2.04 (m, 8H), 2.46 (t, J=11.9 Hz, 1H), 2.83-3.12 (m, 3H), 3.21 (p, J=6.4 Hz, 1H), 3.55 (ddd, J=14.7, 10.6, 3.5 Hz, 2H), 3.98 (dd, J=10.5, 5.5 Hz, 1H), 4.05-4.19 (m, 4H), 4.24 (t, J=7.9 Hz, 1H), 5.66 (dd, J=10.1, 2.1 Hz, 1H), 5.77 (q, J=6.9 Hz, 1H), 6.20 (dd, J=17.0, 10.1 Hz, 1H), 6.33 (dd, J=17.0, 2.1 Hz, 1H), 6.61 (d, J=1.8 Hz, 1H), 6.66 (t, J=7.1 Hz, 2H), 6.89 (d, J=8.2 Hz, 1H), 7.06 (s, 1H), 8.04 (d, J=5.2 Hz, 1H).

MP: 256.7° C. (Mettler Toledo MP50), uncorrected.

OR: +14.25° (589 nm, c 0.1067 w/v, CHCl₃, 23° C.).

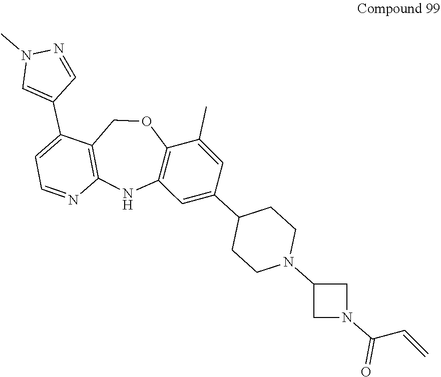 [SMILES: C=CC(=O)N1CC(N2CCC(c3cc(C)c4c(c3)Nc3nccc(-c5cnn(C)c5)c3CO4)CC2)C1]

Compound 99 was synthesized in a similar manner as Compound 164 using Intermediate 404 instead of Intermediate 50.

LC MS: confirms the MW (RT: 1.826, [M+H]⁺: 485.1, Method: 2).

¹H NMR: (300 MHz, Chloroform-d) δ (ppm) 8.03 (d, J=5.1 Hz, 1H), 7.57 (s, 1H), 7.48 (s, 1H), 6.96 (br s, 1H), 6.65 (d, J=5.1 Hz, 1H), 6.57 (s, 1H), 6.53 (s, 1H), 6.40-6.27 (m, 1H), 6.19 (dd, J=17.0, 10.1 Hz, 1H), 5.73-5.61 (m, 1H), 5.11 (s, 2H), 4.24 (t, J=7.8 Hz, 1H), 4.16-4.07 (m, 2H), 3.99 (s, 3H), 3.98-3.92 (m, 1H), 3.28-3.13 (m, 1H), 3.04-2.85 (m, 2H), 2.46-2.35 (m, 1H), 2.22 (s, 3H), 1.96 (t, J=12.0 Hz, 2H), 1.85 (d, J=12.1 Hz, 2H), 1.81-1.64 (m, 2H).

MP: 149.2° C. (Mettler Toledo FP62), 10° C./min, uncorrected.

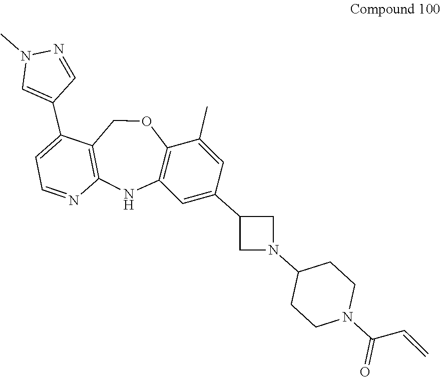 [SMILES: C=CC(=O)N1CCC(N2CC(c3cc(C)c4c(c3)Nc3nccc(-c5cnn(C)c5)c3CO4)C2)CC1]

Compound 100 was synthesized in a similar manner as Compound 164 using Intermediate 407 instead of Intermediate 50.

LC MS: confirms the MW (RT: 1.661, [M+H]⁺: 485.2, Method: 2).

¹H NMR: (300 MHz, Chloroform-d) δ (ppm) 8.04 (d, J=5.1 Hz, 1H), 7.57 (s, 1H), 7.49 (s, 1H), 7.04 (br s, 1H), 6.66 (d, J=5.1 Hz, 1H), 6.64-6.49 (m, 3H), 6.24 (d, J=16.8 Hz, 1H), 5.66 (d, J=10.6 Hz, 1H), 5.12 (s, 2H), 4.27 (d, J=12.5 Hz, 1H), 3.99 (s, 3H), 3.88 (d, J=12.7 Hz, 1H), 3.70 (t, J=7.0 Hz, 2H), 3.63-3.47 (m, 1H), 3.29-3.14 (m, 1H), 3.13-3.03 (m, 3H), 2.39-2.28 (m, 1H), 2.23 (s, 3H), 1.84-1.68 (m, 2H), 1.42-1.21 (m, 2H).

MP: 150.6° C. (Mettler Toledo FP62), 10° C./min; uncorrected.

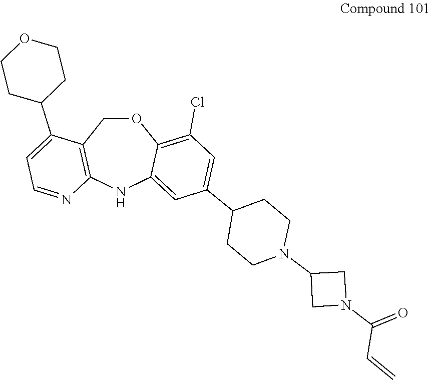 [SMILES: C=CC(=O)N1CC(N2CCC(c3cc(Cl)c4c(c3)Nc3nccc(C5CCOCC5)c3CO4)CC2)C1]

Compound 101 was synthesized in a similar manner as Compound 49 using Intermediate 411 instead of Intermediate 289.

LC MS: confirms the MW (RT: 2.040, [M+H]⁺: 509.2301, Method: 3).

¹H NMR (300 MHz, Chloroform-d) δ (ppm) 1.66-1.80 (m, 4H), 1.84 (q, J=5.3, 4.6 Hz, 2H), 1.90-2.05 (m, 2H), 2.41 (dd, J=13.9, 10.0 Hz, 1H), 2.94 (t, J=13.8 Hz, 2H), 3.12 (td, J=11.9, 6.0 Hz, 1H), 3.17-3.28 (m, 1H), 3.57 (td, J=11.8, 2.0 Hz, 2H), 3.97 (dd, J=10.5, 5.5 Hz, 1H), 4.03-4.17 (m, 6H), 4.24 (t, J=7.9 Hz, 1H), 5.16 (s, 2H), 5.67 (dd, J=10.1, 2.1 Hz, 1H), 6.19 (dd, J=17.0, 10.1 Hz, 1H), 6.33 (dd, J=17.0, 2.1 Hz, 1H), 6.56 (d, J=2.0 Hz, 1H), 6.71 (d, J=5.3 Hz, 1H), 6.77 (d, J=1.9 Hz, 1H), 6.99 (s, 1H), 8.08 (d, J=5.4 Hz, 1H).

MP: 171.4° C. (Mettler Toledo MP50), uncorrected.

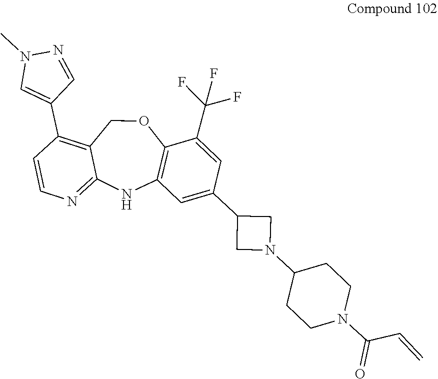 [SMILES: C=CC(=O)N1CCC(N2CC(c3cc4c(c(C(F)(F)F)c3)OCc3c(-c5cnn(C)c5)ccnc3N4)C2)CC1]

Compound 102 was synthesized in a similar manner as Compound 27 using Intermediate 416 instead of Intermediate 222.

LC MS: confirms the MW (RT: 2.203, [M+H]⁺: 539.1, Method: 2).

¹H NMR (300 MHz, Chloroform-d) δ (ppm) 1.21-1.42 (m, 2H), 1.74 (d, J=13.8 Hz, 2H), 2.27-2.42 (m, 1H), 3.08-3.16 (m, 3H), 3.24 (d, J=11.8 Hz, 1H), 3.59 (p, J=6.9 Hz, 1H), 3.70 (t, J=7.2 Hz, 2H), 3.87 (d, J=13.3 Hz, 1H), 4.00 (s, 3H), 4.24 (d, J=13.1 Hz, 1H), 5.17 (s, 2H), 5.67 (dd, J=10.5, 2.0 Hz, 1H), 6.25 (dd, J=16.9, 2.0 Hz, 1H), 6.59 (dd, J=16.8, 10.6 Hz, 1H), 6.73 (d, J=5.2 Hz, 1H), 7.00 (s, 2H), 7.16 (s, 1H), 7.48 (s, 1H), 7.56 (s, 1H), 8.08 (d, J=5.2 Hz, 1H). 159.8° C. (Mettler Toledo MP50), uncorrected.

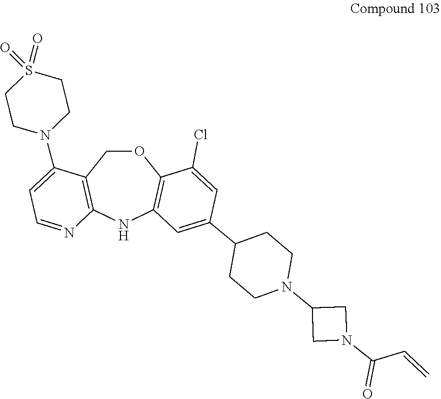 [SMILES: C=CC(=O)N1CC(N2CCC(c3cc(Cl)c4c(c3)Nc3nccc(N5CCS(=O)(=O)CC5)c3CO4)CC2)C1]

Compound 103 was synthesized in a similar manner as Compound 49 using Intermediate 420 instead of Intermediate 289.

LC MS: confirms the MW (RT: 1.623, [M+H]⁺: 558.1925, Method: 3).

¹H NMR (300 MHz, Chloroform-d) δ (ppm) 1.66-1.80 (m, 2H), 1.85 (d, J=12.4 Hz, 2H), 1.92-2.01 (m, 2H), 2.42 (dd, J=13.9, 10.0 Hz, 1H), 2.94 (t, J=13.9 Hz, 2H), 3.12-3.35 (m, 5H), 3.52 (dd, J=7.0, 3.6 Hz, 4H), 3.96 (dd, J=10.6, 5.4 Hz, 1H), 4.04-4.18 (m, 2H), 4.24 (t, J=7.9 Hz, 1H), 5.11 (s, 2H), 5.67 (dd, J=10.1, 2.0 Hz, 1H), 6.19 (dd, J=17.0, 10.2 Hz, 1H), 6.33 (dd, J=17.0, 2.1 Hz, 1H), 6.46 (d, J=5.5 Hz, 1H), 6.57 (s, 1H), 6.81 (d, J=2.0 Hz, 1H), 6.93 (s, 1H), 8.05 (d, J=5.5 Hz, 1H).

MP: 224.9° C. (Mettler Toledo MP50), uncorrected.

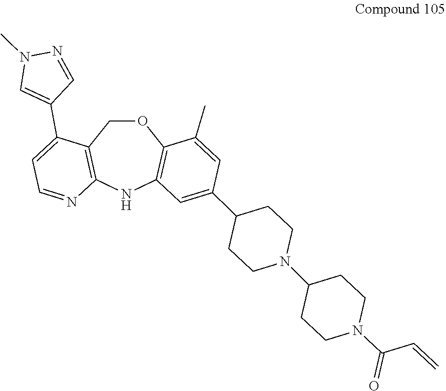 [SMILES: C=CC(=O)N1CCC(N2CCC(c3cc(C)c4c(c3)Nc3nccc(-c5cnn(C)c5)c3CO4)CC2)CC1]

Compound 105 was synthesized in a similar manner as Compound 164 using Intermediate 421 instead of Intermediate 50.

LC MS: confirms the MW (RT: 1.894, [M+H]⁺: 513.0, Method: 2).

¹H NMR: (300 MHz, Chloroform-d) δ (ppm) 8.03 (d, J=5.1 Hz, 1H), 7.56 (s, 1H), 7.47 (s, 1H), 6.94 (s, 1H), 6.70-6.49 (m, 4H), 6.26 (d, J=16.8 Hz, 1H), 5.67 (d, J=10.6 Hz, 1H), 5.11 (s, 2H), 4.72 (d, J=12.0 Hz, 1H), 4.06 (d, J=12.9 Hz, 1H), 3.99 (s, 3H), 3.12-2.96 (m, 3H), 2.74-2.49 (m, 2H), 2.44-2.25 (m, 3H), 2.22 (s, 3H), 1.99-1.60 (m, 6H), 1.57-1.45 (m, 2H).

MP: 187.3° C. (Mettler Toledo FP62), 10° C./min, uncorrected.

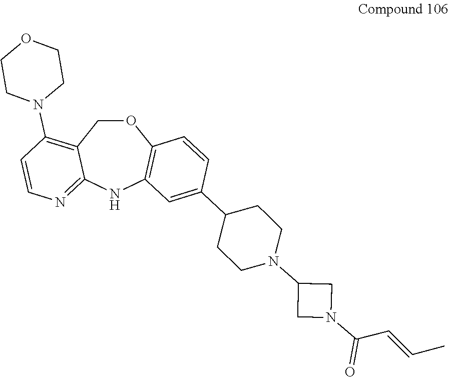 [SMILES: C/C=C/C(=O)N1CC(N2CCC(c3ccc4c(c3)Nc3nccc(N5CCOCC5)c3CO4)CC2)C1]

Compound 106 was synthesized in a similar manner as Compound 3 using Intermediate 15 instead of Intermediate 10 and crotonic acid [CAS: 107-93-7] instead of acrylic acid.

LC MS: confirms the MW (RT: 2.65, [M+H]⁺: 490.3, Method: 1).

¹H NMR: (500 MHz, DMSO-d₆, 22° C.): δ (ppm) 8.94 (s, 1H), 7.97 (d, J=5.4 Hz, 1H), 7.08 (d, J=2.2 Hz, 1H), 6.81 (d, J=8.2 Hz, 1H), 6.61-6.67 (m, 1H), 6.58 (dd, J=8.2, 2.2 Hz, 1H), 6.44 (d, J=5.7 Hz, 1H), 5.99 (dq, J=15.1, 1.7 Hz, 1H), 4.96 (s, 2H), 4.19 (br t, J=7.9 Hz, 1H), 3.82-4.04 (m, 2H), 3.67-3.78 (m, 5H), 3.06-3.18 (m, 1H), 2.84-2.95 (m, 5H), 2.33-2.41 (m, 1H), 1.85-1.92 (m, 2H), 1.82 (dd, J=6.9, 1.6 Hz, 3H), 1.68-1.77 (m, 2H), 1.57 ppm (q, J-=12.1 Hz, 2H)

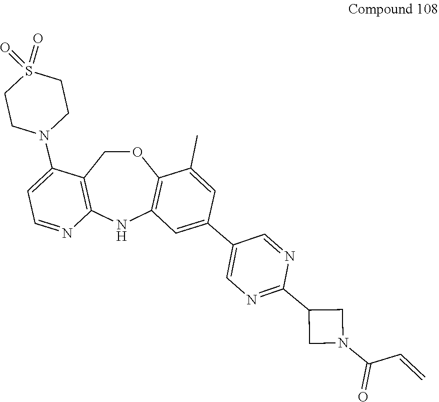 [SMILES: C=CC(=O)N1CC(c2ncc(-c3cc(C)c4c(c3)Nc3nccc(N5CCS(=O)(=O)CC5)c3CO4)cn2)C1]

Compound 108 was synthesized in a similar manner as Compound 173 using Intermediate 428 instead of Intermediate 65.

LC MS: confirms the MW (RT: 2.116, [M+H]⁺: 533.1 Method: 2).

¹H NMR (300 MHz, DMSO-d₆) δ (ppm) 9.17 (s, 1H), 9.00 (s, 2H), 8.01 (d, J=5.4 Hz, 1H), 7.44 (s, 1H), 7.10 (s, 1H), 6.58 (d, J=5.5 Hz, 1H), 6.37 (dd, J=17.0, 10.3 Hz, 1H), 6.13 (d, J=15.2 Hz, 1H), 5.69 (d, J=12.0 Hz, 1H), 5.11 (s, 2H), 4.65 (t, J=8.1 Hz, 1H), 4.53-4.41 (m, 1H), 4.35 (t, J=8.6 Hz 1H), 4.23-4.07 (m, 2H), 3.36 (s, 8H), 2.28 (br s, 3H)

MP: 274.9° C. (Mettler Toledo FP62), 10° C./min; uncorrected.

### Compound 109 and Compound 110

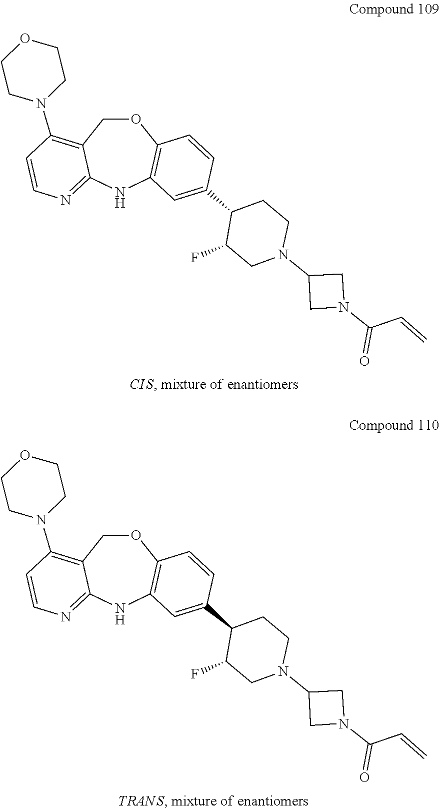 [SMILES: C=CC(=O)N1CC(N2CC[C@@H](c3ccc4c(c3)Nc3nccc(N5CCOCC5)c3CO4)[C@@H](F)C2)C1 ; C=CC(=O)N1CC(N2CC[C@H](c3ccc4c(c3)Nc3nccc(N5CCOCC5)c3CO4)[C@@H](F)C2)C1]

Compound 109 and Compound 110 were synthesized in a similar manner as Compound 354 starting with Intermediate 431 instead of Intermediate 354. The isomers mixture was purified by reverse phase chromatography (Phenomenex Gemini C18 30×100 mm 5 μm; gradient from 72% H2O−28% ACN-MeOH to 36% H2O−64% ACN-MeOH−[0.1% HCOOH]).

LC MS (Compound 109): confirms the MW (RT: 1.716, [M+H]⁺: 494, Method: 2).

¹H NMR (Compound 109): (300 MHz, Chloroform-d) δ (ppm) 7.98 (d, J=5.6 Hz, 1H), 7.00 (br s, 1H), 6.92 (d, J=8.2 Hz, 1H), 6.74 (s, 1H), 6.70 (d, J=8.5 Hz, 1H), 6.43-6.27 (m, 2H), 6.25-6.08 (m, 1H), 5.66 (d, J=10.2 Hz, 1H), 5.05 (s, 2H), 4.75-4.44 (m, 1H), 4.29-4.14 (m, 1H), 4.14-4.02 (m, 2H), 4.00-3.83 (m, 5H), 3.28-3.16 (m, 1H), 3.08-3.00 (m, 4H), 3.00-2.77 (m, 3H), 2.07 (t, J=10.8 Hz, 2H), 2.01-1.81 (m, 2H).

MP (Compound 109): 115.5° C. (Mettler Toledo FP62), 10° C./min, uncorrected.

LC MS (Compound 110): confirms the MW (RT: 1.967, [M+H]⁺: 494, Method: 2).

¹H NMR (Compound 110): (300 MHz, Chloroform-d) δ (ppm) 7.98 (d, J=5.5 Hz, 1H), 6.97 (br s, 1H), 6.92 (d, J=8.3 Hz, 1H), 6.70 (s, 1H), 6.69 (d, J=7.4 Hz, 1H), 6.44-6.29 (m, 2H), 6.19 (dd, J=17.0, 10.2 Hz, 1H), 5.68 (d, J=10.2 Hz, 1H), 5.05 (s, 2H), 4.59 (dtd, J=48.5, 9.8, 4.6 Hz, 1H), 4.25 (t, J=7.4 Hz, 1H), 4.20-4.03 (m, 2H), 3.99-3.92 (m, 1H), 3.90-3.84 (m, 4H), 3.39-3.14 (m, 2H), 3.10-2.96 (m, 4H), 2.85 (t, J=12.6 Hz, 1H), 2.66-2.52 (m, 1H), 2.08-1.95 (m, 2H), 1.87-1.71 (m, 2H).

MP (Compound 110): 162.2° C. (Mettler Toledo FP62), 10° C./min, uncorrected.

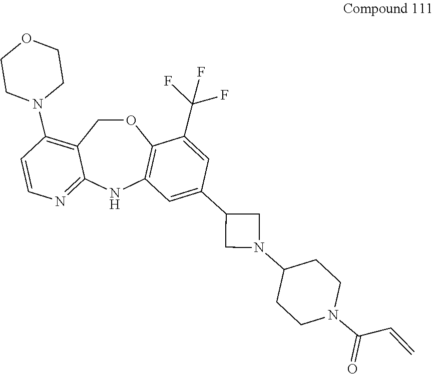 [SMILES: C=CC(=O)N1CCC(N2CC(c3cc4c(c(C(F)(F)F)c3)OCc3c(N5CCOCC5)ccnc3N4)C2)CC1]

Compound 111 was prepared using a procedure similar to Compound 263, starting from Intermediate 433 instead of Intermediate 57.

LC MS: confirms the MW (RT: 1.980, [M+H]⁺: 544.0, Method: 2).

¹H NMR (300 MHz, Chloroform-d) δ (ppm) 1.32 (dtd, J=13.2, 9.3, 3.9 Hz, 2H), 1.68-1.82 (m, 2H), 2.35 (dt, J=8.9, 4.7 Hz, 1H), 2.97 (t, J=4.6 Hz, 4H), 3.11 (t, J=6.7 Hz, 2H), 3.15-3.31 (m, 2H), 3.58 (p, J=6.9 Hz, 1H), 3.69 (t, J=7.1 Hz, 2H), 3.84-3.98 (m, 5H), 4.23 (d, J=13.2 Hz, 1H), 5.10 (s, 2H), 5.67 (dd, J=10.6, 2.0 Hz, 1H), 6.25 (dd, J=16.8, 2.0 Hz, 1H), 6.44 (d, J=5.6 Hz, 1H), 6.58 (dd, J=16.9, 10.5 Hz, 1H), 6.92-7.06 (m, 3H), 8.02 (d, J=5.6 Hz, 1H).

MP: 215.0° C. (Mettler Toledo MP50), uncorrected.

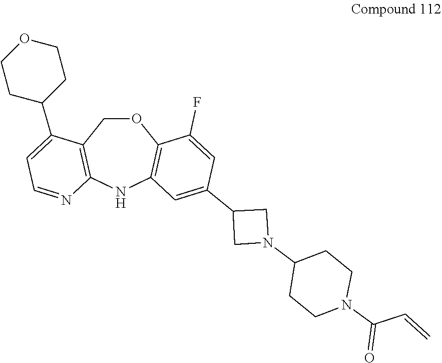 [SMILES: C=CC(=O)N1CCC(N2CC(c3cc(F)c4c(c3)Nc3nccc(C5CCOCC5)c3CO4)C2)CC1]

Compound 112 was prepared using a procedure analogous to Compound 173, starting from Intermediate 436 instead of Intermediate 65.

LC MS: confirms the MW (RT: 1.943, [M+H]⁺: 493.1, Method: 2).

¹H NMR (300 MHz, DMSO-d₆) δ (ppm) 9.28 (s, 1H), 8.08 (d, J=5.2 Hz, 1H), 7.05 (s, 1H), 6.88-6.72 (m, 2H), 6.62 (d, J=11.2 Hz, 1H), 6.06 (dd, J=10.5, 2.1 Hz, 1H), 5.64 (dd, J=10.5, 2.1 Hz, 1H), 5.14 (s, 2H), 4.02-3.77 (m, 4H), 3.53 (t, J=10.6 Hz, 4H), 3.46-3.35 (m, 1H), 3.28-3.15 (m, 2H), 3.10 (d, J=9.8 Hz, 1H), 3.01 (t, J=6.5 Hz, 2H), 2.38-2.27 (m, 1H), 1.76-1.50 (m, 6H), 1.23-1.04 (m, 2H)

MP: 109.6° C. (Mettler Toledo FP 62), 10° C./min, uncorrected.

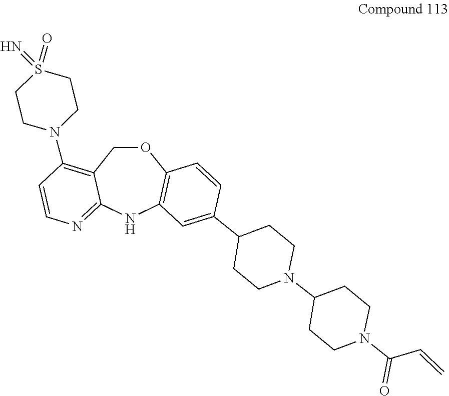 [SMILES: C=CC(=O)N1CCC(N2CCC(c3ccc4c(c3)Nc3nccc(N5CCS(=N)(=O)CC5)c3CO4)CC2)CC1]

Potassium tert-butoxide (200 μl, 0.2 mmol) was added to a solution of Intermediate 438 (73 mg, 0.1 mmol) in THF (4 mL) at room temperature. The reaction mixture was stirred at room temperature 1 h. Potassium tert-butoxide (120 μl, 0.12 mmol) was added again and the reaction mixture was stirred for an additional h. The reaction mixture was evaporated until dryness. The residue was purified via column chromatography (Stationary phase: irregular SiOH 15-40 μm 12 g interchim, Mobile phase: 97/3 to 90/10/0.1 CMA). A second purification was performed via column chromatography (Stationary phase: irregular SiOH 15-40 μm 12 g interchim, Mobile phase: 97/3 to 90/10/0.1 CMA) yielding Compound 113 (8 mg, yield: 14%).

LC MS: confirms the MW (RT: 1.81, [M+H]⁺: 551.5, Method: 1).

¹H NMR: (500 MHz, DMSO-d₆, 24° C.): δ (ppm) 8.96 (s, 1H), 7.96 (d, J=5.7 Hz, 1H), 7.08 (d, J=2.2 Hz, 1H), 6.77-6.84 (m, 2H), 6.58 (dd, J=8.2, 1.9 Hz, 1H), 6.51 (d, J=5.7 Hz, 1H), 6.07 (dd, J=16.7, 2.5 Hz, 1H), 5.75 (s, 1H), 5.63-5.67 (m, 1H), 4.99 (s, 2H), 4.45 (br d, J=11.7 Hz, 1H), 4.08 (br d, J=13.2 Hz, 1H), 3.77 (s, 1H), 3.19-3.29 (m, 3H), 3.11-3.19 (m, 2H), 3.02 (br t, J=12.1 Hz, 1H), 2.92 (br d, J=11.3 Hz, 2H), 2.57-2.69 (m, 1H), 2.32 (tt, J=12.1, 3.5 Hz, 1H), 2.16-2.27 (m, 2H), 1.79 (br d, J=11.7 Hz, 2H), 1.70 (br d, J=12.6 Hz, 2H), 1.53 (qd, J=12.3, 3.5 Hz, 2H), 1.26-1.39 ppm (m, 2H)

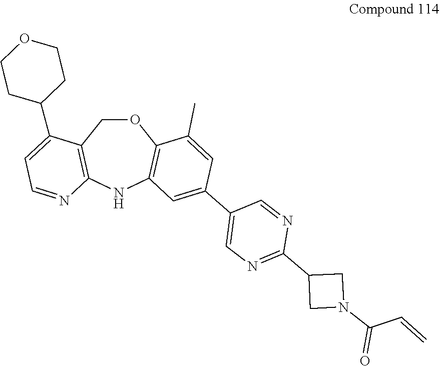 [SMILES: C=CC(=O)N1CC(c2ncc(-c3cc(C)c4c(c3)Nc3nccc(C5CCOCC5)c3CO4)cn2)C1]

Compound 114 was prepared using a procedure analogous to Compound 173, starting from Intermediate 443 instead of Intermediate 65.

LC MS: confirms the MW (RT: 2.603, [M+H]⁺: 484.2, Method: 2).

¹H NMR (300 MHz, CDCl₃) δ (ppm) 8.11 (d, J=5.3 Hz, 1H), 6.92 (d, J=6.4 Hz, 2H), 6.84 (s, 1H), 6.77 (d, J=5.3 Hz, 1H), 6.34 (d, J=16.9 Hz, 1H), 6.19 (dd, J=17.0, 10.1 Hz, 1H), 5.67 (d, J=10.1 Hz, 1H), 5.21 (s, 2H), 4.24 (t, J=7.9 Hz, 1H), 4.15-4.09 (m, 4H), 3.96 (dd, J=10.3, 5.4 Hz, 1H), 3.59 (t, J=11.6 Hz, 2H), 3.30-3.06 (m, 2H), 3.04-2.86 (m, 2H), 2.45 (t, J=12.0 Hz, 1H), 2.05-1.65 (m, 10H)

MP: 241.1° C. (Mettler Toledo FP62), 10° C./min; uncorrected.

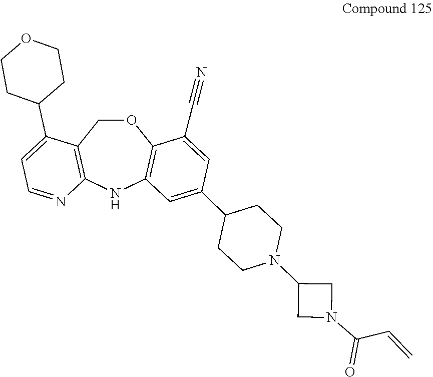 [SMILES: C=CC(=O)N1CC(N2CCC(c3cc(C#N)c4c(c3)Nc3nccc(C5CCOCC5)c3CO4)CC2)C1]

Compound 125 was synthesized in a similar manner as Compound 173 using Intermediate 445 instead of Intermediate 65.

LC MS: confirms the MW (RT: 2.000, [M+H]⁺: 500.3264, Method: 3).

¹H NMR (300 MHz, CDCl₃) δ (ppm) 8.11 (d, J=5.3 Hz, 1H), 6.92 (d, J=6.4 Hz, 2H), 6.84 (s, 1H), 6.77 (d, J=5.3 Hz, 1H), 6.34 (d, J=16.9 Hz, 1H), 6.19 (dd, J=17.0, 10.1 Hz, 1H), 5.67 (d, J=10.1 Hz, 1H), 5.21 (s, 2H), 4.24 (t, J=7.9 Hz, 1H), 4.15-4.09 (m, 4H), 3.96 (dd, J=10.3, 5.4 Hz, 1H), 3.59 (t, J=11.6 Hz, 2H), 3.30-3.06 (m, 2H), 3.04-2.86 (m, 2H), 2.45 (t, J=12.0 Hz, 1H), 2.05-1.65 (m, 10H)

MP: 256.9° C. (Mettler Toledo FP62), 10° C./min; uncorrected.

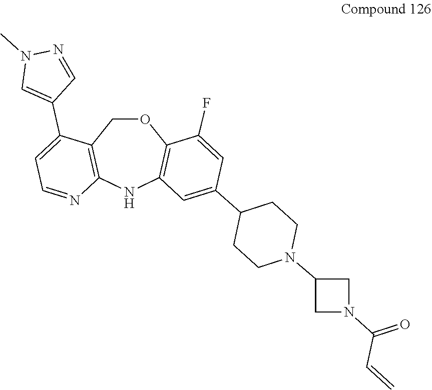 [SMILES: C=CC(=O)N1CC(N2CCC(c3cc(F)c4c(c3)Nc3nccc(-c5cnn(C)c5)c3CO4)CC2)C1]

Compound 126 was synthesized in a similar manner as Compound 164 using Intermediate 448 instead of Intermediate 50.

LC MS: confirms the MW (RT: 1.833, [M+H]⁺: 489.3203, Method: 3).

¹H NMR (300 MHz, CDCl₃) δ (ppm) 8.06 (s, 1H), 7.59 (s, 1H), 7.53 (s, 1H), 7.11 (s, 1H), 6.73 (s, 1H), 6.49 (d, J=18.3 Hz, 2H), 6.39-6.09 (m, 2H), 5.66 (d, J=9.4 Hz, 1H), 5.17 (s, 2H), 4.30-3.92 (m, 2H), 3.98 (s, 3H), 3.20 (s, 1H), 2.94 (s, 2H), 2.41 (s, 1H), 2.10-1.57 (m, 8H)

MP: 196.0° C. (Mettler Toledo FP62), 10° C./min. uncorrected.

### Compound 127 and Compound 128

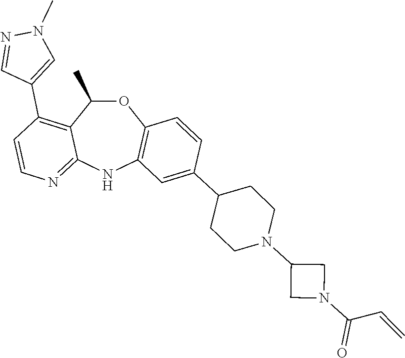 [SMILES: C=CC(=O)N1CC(N2CCC(c3ccc4c(c3)Nc3nccc(-c5cnn(C)c5)c3[C@@H](C)O4)CC2)C1]

Compound 127: (*R), pure stereoisomer but absolute stereochemistry undetermined

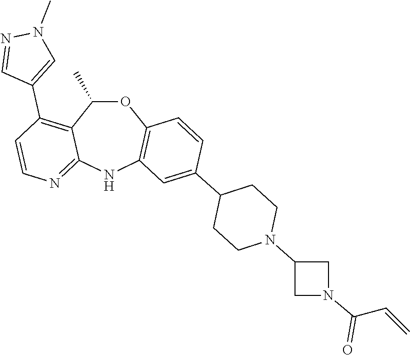 [SMILES: C=CC(=O)N1CC(N2CCC(c3ccc4c(c3)Nc3nccc(-c5cnn(C)c5)c3[C@H](C)O4)CC2)C1]

Compound 128: (*S), pure stereoisomer but absolute stereochemistry undetermined Compound 127 and Compound 128 was synthesized in a similar manner as Compound 27 using Intermediate 450 instead of Intermediate 222. The isomeric mixture was then separated by chiral SFC (Stationary phase: CHIRALCEL OD-H 5 μm 250*30 mm, Mobile phase: 50% CO₂, 50% MeOH with 0.6% Et₃N) to yield Compound 127 (98 mg, yield 15%) and Compound 128 (103 mg, yield 16%) LC MS (Compound 127): confirms the MW (RT: 1.733, [M+H]⁺: 485.2144, Method: 3).

¹H NMR (Compound 127): (300 MHz, Chloroform-d) δ (ppm) 1.35 (d, J=7.0 Hz, 3H), 1.77 (td, J=12.4, 3.4 Hz, 2H), 1.87 (s, 2H), 1.90-2.05 (m, 2H), 2.38-2.55 (m, 1H), 2.96 (t, J=13.9 Hz, 2H), 3.21 (ddd, J=12.7, 7.2, 5.5 Hz, 1H), 3.98 (s, 4H), 4.07-4.17 (m, 2H), 4.25 (t, J=7.9 Hz, 1H), 5.60-5.77 (m, 2H), 6.14-6.25 (m, 1H), 6.33 (dd, J=17.0, 2.1 Hz, 1H), 6.59-6.65 (m, 2H), 6.67 (dd, J=8.2, 2.0 Hz, 1H), 6.86 (d, J=8.1 Hz, 1H), 7.14 (s, 1H), 7.46 (s, 1H), 7.56 (d, J=0.8 Hz, 1H), 8.03 (d, J=5.1 Hz, 1H).

MP (Compound 127): 221.7° C. (Mettler Toledo MP50), 10° C./min, uncorrected.

OR (Compound 127): +4.6135° (589 nm, c 0.138 w/v, MeOH, 23.00° C.).

LC MS (Compound 128): confirms the MW (RT: 1.720, [M+H]⁺: 485.2227, Method: 3).

¹H NMR (Compound 128): (300 MHz, Chloroform-d) δ (ppm) 1.35 (d, J=7.0 Hz, 3H), 1.74-1.84 (m, 2H), 1.83-2.07 (m, 4H), 2.39-2.52 (m, 1H), 2.95 (t, J=13.8 Hz, 2H), 3.20 (h, J=5.7, 5.3 Hz, 1H), 3.98 (s, 4H), 4.06-4.18 (m, 2H), 4.24 (t, J=7.9 Hz, 1H), 5.61-5.75 (m, 2H), 6.19 (dd, J=17.0, 10.1 Hz, 1H), 6.33 (dd, J=17.0, 2.1 Hz, 1H), 6.57-6.71 (m, 3H), 6.86 (d, J=8.1 Hz, 1H), 7.17 (s, 1H), 7.46 (s, 1H), 7.56 (s, 1H), 8.03 (d, J=5.1 Hz, 1H).

MP (Compound 128): 223.3° C. (Mettler Toledo MP50), 10° C./min, uncorrected.

OR (Compound 128): −9.5853° (589 nm, c 0.136667 w/v, MeOH, 23.00° C.).

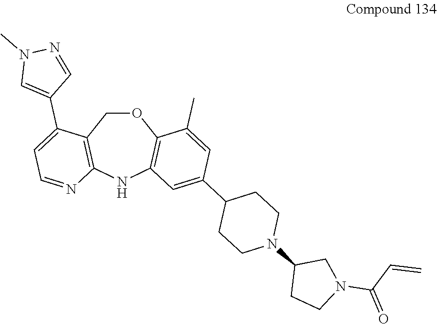 [SMILES: C=CC(=O)N1CC[C@@H](N2CCC(c3cc(C)c4c(c3)Nc3nccc(-c5cnn(C)c5)c3CO4)CC2)C1]

(*R), pure stereoisomer but absolute stereochemistry undetermined Compound 134 was synthesized in a similar manner as Compound 164 using Intermediate 454 instead of Intermediate 50.

LC MS: confirms the MW (RT: 1.79, [M+H]⁺: 499.2870, Method: 3).

¹H NMR (300 MHz, CDCl₃) δ (ppm) 8.03 (d, J=5.1 Hz, 1H), 7.57 (s, 1H), 7.48 (s, 1H), 6.95 (s, 1H), 6.65 (d, J=5.0 Hz, 1H), 6.57-6.46 (m 2H), 6.43-6.34 (m, 2H), 5.67 (d, J=9.0 Hz, 1H), 5.11 (s, 2H), 4.04-3.95 (m, 0.5H), 3.99 (s 2H), 3.85 (t, J=9.1 Hz, 1H), 3.76 (t, J=9.5 Hz, 0.5H), 3.58-3.25 (m, 2H), 3.21-2.73 (m, 3H), 2.39 (t, J=10.2 Hz, 1H), 2.30-2.05 (m, 3H), 2.22 (s, 3H), 2.02-1.69 (m, 6H)

MP: 220.6° C. (Mettler Toledo FP62), 10° C./min, uncorrected.

OR: +13.1111° (589 nm, c 0.2700 w/v, MeOH, 23.0° C.).

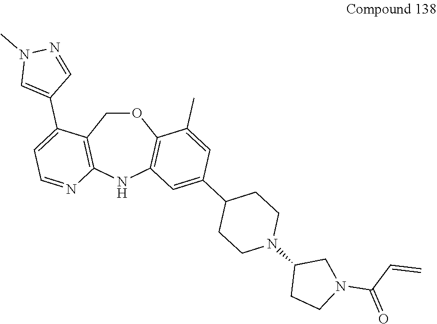 [SMILES: C=CC(=O)N1CC[C@H](N2CCC(c3cc(C)c4c(c3)Nc3nccc(-c5cnn(C)c5)c3CO4)CC2)C1]

(*S), pure stereoisomer but absolute stereochemistry undetermined Compound 138 was synthesized in a similar manner as Compound 164 using Intermediate 455 instead of Intermediate 50.

LC MS: confirms the MW (RT: 1.800, [M+H]⁺: 499.2880, Method: 3).

¹H NMR (300 MHz, CDCl₃) δ (ppm) 8.03 (d, J=5.1 Hz, 1H), 7.57 (s, 1H), 7.48 (s, 1H), 6.95 (s, 1H), 6.65 (d, J=5.0 Hz, 1H), 6.55 (d, J=12.5 Hz, 1H), 6.47-6.31 (m, 1H), 5.79-5.58 (m, 1H), 5.11 (s, 2H), 3.9.7 (t, J=9.2 Hz, 0.5H), 3.99 (s, 3H), 3.85 (t, J=9.1 Hz, 1H), 3.76 (t, J=9.2 Hz, 0.5H), 3.62-3.22 (m, 2H), 3.12 (d, J=10.6 Hz, 1H), 3.07-2.73 (m, 1H), 2.48-2.32 (m, 1H), 2.30-2.06 (m, 4H), 2.22 (s, 3H), 1.98-1.78 (m, 4H), 1.78-1.63 (m, 3H).

MP: 152.2° C. (Mettler Toledo FP62), 10° C./min, uncorrected.

OR: −13.2518° (589 nm, c 0.2727 w/v, MeOH, 23.0° C.).

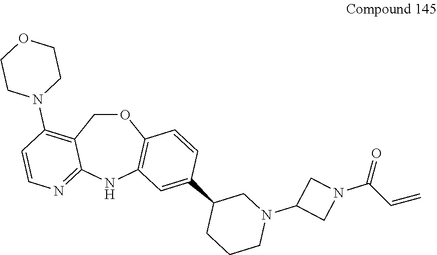 [SMILES: C=CC(=O)N1CC(N2CCC[C@@H](c3ccc4c(c3)Nc3nccc(N5CCOCC5)c3CO4)C2)C1]

(*S), pure stereoisomer but absolute stereochemistry undetermined Compound 145 was synthesized in a similar manner as Compound 3 using Intermediate 392 instead of Intermediate 10.

LC MS: confirms the MW (RT: 2.45, [M+H]⁺: 476.6, Method: 10).

¹H NMR: (500 MHz, DMSO-d₆, 24° C.): δ (ppm) 8.83 (d, J=4.7 Hz, 1H), 7.90 (d, J=5.4 Hz, 1H), 7.04 (t, J=1.9 Hz, 1H), 6.74 (d, J=8.2 Hz, 1H), 6.53 (br d, J=8.2 Hz, 1H), 6.37 (d, J=5.7 Hz, 1H), 6.22 (ddd, J=17.0, 11.8, 10.2 Hz, 1H), 5.98-6.11 (m, 1H), 5.54-5.63 (m, 1H), 4.89 (s, 2H), 4.08-4.21 (m, 1H), 3.92-4.06 (m, 1H), 3.86 (ddd, J=13.5, 10.2, 7.3 Hz, 1H), 3.64-3.72 (m, 5H), 3.03-3.12 (m, 1H), 2.82-2.85 (m, 3H), 2.67-2.80 (m, 2H), 2.48-2.55 (m, 1H), 1.62-1.79 (m, 4H), 1.43-1.59 (m, 1H), 1.24-1.41 ppm (m, 1H)

OR: −78.18° (589 nm, c 0.22 w/v %, DMF, 20° C.).

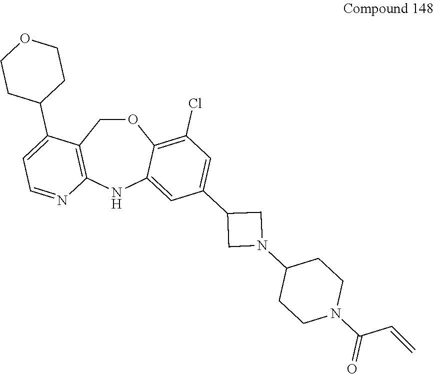 [SMILES: C=CC(=O)N1CCC(N2CC(c3cc(Cl)c4c(c3)Nc3nccc(C5CCOCC5)c3CO4)C2)CC1]

Compound 148 was synthesized in a similar manner as Compound 30 using Intermediate 462 instead of Intermediate 232.

LC MS: confirms the MW (RT: 1.89, [M+H]⁺: 509.2, Method: 4).

¹H NMR: (400 MHz, Chloroform-d) δ (ppm) 1.13 (d, J=6.01 Hz, 1H) 1.67-1.78 (m, 4H) 1.77-1.91 (m, 2H) 2.34 (dq, J=8.44, 4.20 Hz, 1H) 3.01-3.15 (m, 4H) 3.16-3.30 (m, 1H) 3.45-3.62 (m, 3H) 3.63-3.74 (m, 2H) 3.81-3.95 (m, 1H) 4.05-4.14 (m, 2 H) 4.18-4.33 (m, 1H) 5.16 (s, 2H) 5.23-5.42 (m, 1H) 5.67 (dd, J=10.52, 1.96 Hz, 1 H) 6.25 (dd, J=16.88, 2.08 Hz, 1H) 6.58 (dd, J=16.76, 10.52 Hz, 1H) 6.67 (d, J=2.08 Hz, 1H) 6.72 (d, J=5.32 Hz, 1H) 6.84 (d, J=1.85 Hz, 1H) 6.94-7.02 (m, 1H) 8.09 (d, J=5.32 Hz, 1H).

### Compound 149 and Compound 150

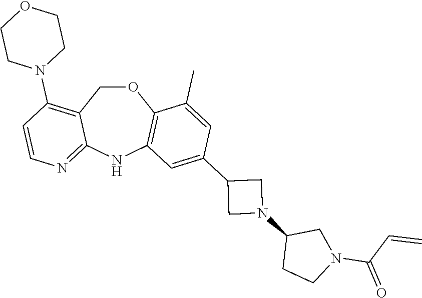 [SMILES: C=CC(=O)N1CC[C@@H](N2CC(c3cc(C)c4c(c3)Nc3nccc(N5CCOCC5)c3CO4)C2)C1] [1499] Compound 149: (*R), pure stereoisomer but absolute stereochemistry undetermined

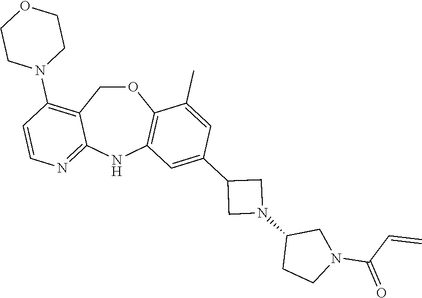 [SMILES: C=CC(=O)N1CC[C@H](N2CC(c3cc(C)c4c(c3)Nc3nccc(N5CCOCC5)c3CO4)C2)C1]

Compound 150: (*S), pure stereoisomer but absolute stereochemistry undetermined

Compound 149 and Compound 150 were synthesized in a similar manner as Compound 272 using Intermediate 463 instead of Intermediate 89. The isomers were separated by chiral SFC (Stationary phase: CHIRALCEL OD-H 5 μm 250*30 mm, Mobile phase: 50% CO₂, 50% EtOH with 0.3% Et₃N).

LC MS (Compound 149): confirms the MW (RT: 2.46, [M+H]⁺: 476.4, Method: 1).

¹H NMR (Compound 149): (400 MHz, DMSO-d₆, 24° C.): δ (ppm) 8.84 (d, J=3.9 Hz, 1H), 7.89 (d, J=5.4 Hz, 1H), 6.93 (d, J=1.7 Hz, 1H), 6.44-6.56 (m, 2H), 6.36 (d, J=5.4 Hz, 1H), 6.05 (dt, J=16.9, 2.2 Hz, 1H), 5.58 (ddd, J=10.3, 5.4, 2.4 Hz, 1H), 4.91 (s, 2H), 3.61-3.76 (m, 4H), 3.29-3.55 (m, 6H), 3.14-3.21 (m, 1H), 2.85-2.99 (m, 3H), 2.74-2.85 (m, 4H), 2.10 (s, 3H), 1.58-1.84 ppm (m, 2H)

OR (Compound 149): −46.35° (365 nm, c 0.192 w/v %, DMF, 20° C.).

LC MS (Compound 150): confirms the MW (RT: 2.45, [M+H]⁺: 476.4, Method: 1).

¹H NMR (Compound 150): (400 MHz, DMSO-d₆, 24° C.): δ (ppm) 8.83 (d, J=3.9 Hz, 1H), 7.89 (d, J=5.6 Hz, 1H), 6.93 (d, J=1.7 Hz, 1H), 6.43-6.57 (m, 2H), 6.36 (d, J=5.6 Hz, 1H), 6.05 (dt, J=16.8, 2.2 Hz, 1H), 5.58 (ddd, J=10.3, 5.4, 2.4 Hz, 1H), 4.91 (s, 2H), 3.58-3.78 (m, 4H), 3.29-3.55 (m, 6H), 3.14-3.20 (m, 1H), 2.87-2.99 (m, 3H), 2.74-2.85 (m, 4H), 2.10 (s, 3H), 1.58-1.84 ppm (m, 2H)

OR (Compound 150): +30.67° (365 nm, c 0.238 w/v %, DMF, 20° C.).

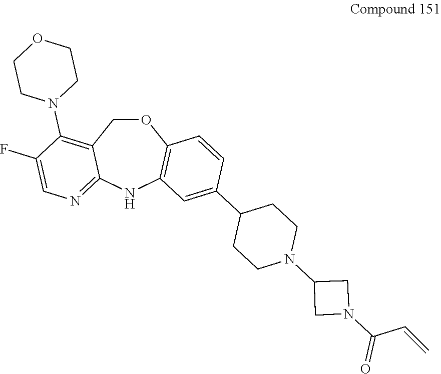 [SMILES: C=CC(=O)N1CC(N2CCC(c3ccc4c(c3)Nc3ncc(F)c(N5CCOCC5)c3CO4)CC2)C1]

Acrylic acid (19 μl, 0.28 mmol, 1.0 eq.) in DCM (3 mL) was added dropwise to a solution of Intermediate 465 (132 mg, 0.28 mmol, 1.0 eq.), Et₃N (0.15 mL, 1.1 mmol, 4.0 eq.), EDCI HCl (64 mg, 0.33 mmol, 1.2 eq.) and 1-hydroxybenzotriazole (45 mg, 0.33 mmol, 1.2 eq.) in DCM (3 mL) at 5° C. The reaction mixture was stirred at room temperature overnight. H₂O and DCM were added, the reaction mixture was extracted. The organic layer was separated, dried over MgSO₄, filtered, and evaporated. A purification was performed via column chromatography (Stationary phase: irregular SiOH 40 μm 25 g, Mobile phase: 97/3/0.1 to 90/10/0.1 CMA). A second purification was performed via reverse phase column chromatography (Stationary phase: YMC-actus Triart C18 10 μm 30*150 mm, Mobile phase: Gradient from 65% NH₄HCO₃ 0.2%, 35% ACN to 25% NH₄HCO₃ 0.2%, 75% ACN) yielding Compound 151 (20 mg, yield: 15%).

LC MS: confirms the MW (RT: 2.65, [M+H]⁺: 494.4, Method: 1).

¹H NMR: (500 MHz, DMSO-d₆, 22° C.): δ (ppm) 8.97 (s, 1H), 8.00 (d, J=3.8 Hz, 1H), 7.06 (d, J=1.9 Hz, 1H), 6.81 (d, J=8.2 Hz, 1H), 6.58 (dd, J=8.4, 2.0 Hz, 1H), 6.31 (dd, J=17.0, 10.4 Hz, 1H), 6.10 (dd, J=17.0, 2.5 Hz, 1H), 5.65-5.68 (m, 1H), 5.04 (s, 2H), 4.24 (t, J=8.0 Hz, 1H), 4.03 (dd, J=8.8, 5.4 Hz, 1H), 3.94 (dd, J=10.4, 7.3 Hz, 1H), 3.69-3.76 (m, 5H), 3.07-3.16 (m, 5H), 2.84-2.93 (m, 2H), 2.52-2.53 (m, 1H), 2.35-2.38 (m, 1H), 1.85-1.95 (m, 2H), 1.73 (br d, J=12.6 Hz, 2H), 1.57 ppm (qd, J=12.3, 3.5 Hz, 2H).

MP: 168.34° C./−50.31 J/g (DSC: 25° C. to 350° C./10° C. min/40 μl Al)

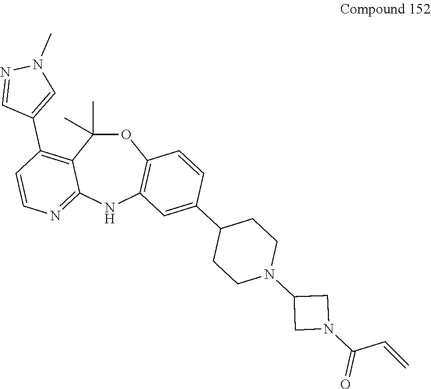 [SMILES: C=CC(=O)N1CC(N2CCC(c3ccc4c(c3)Nc3nccc(-c5cnn(C)c5)c3C(C)(C)O4)CC2)C1]

Compound 152 was synthesized in a similar manner as Compound 263 using Intermediate 470 instead of Intermediate 57.

LC MS: confirms the MW (RT: 1.797, [M+H]⁺: 499.2, Method: 2).

¹H NMR: (400 MHz, Chloroform-d) δ (ppm) 1.41 (s, 6H), 1.77 (td, J=12.5, 3.4 Hz, 2H), 1.83-2.04 (m, 4H), 2.39-2.56 (m, 1H), 2.87-3.04 (m, 2H), 3.21 (p, J=6.2 Hz, 1H), 3.97 (s, 4H), 4.06-4.17 (m, 2H), 4.24 (t, J=7.9 Hz, 1H), 5.66 (dd, J=10.1, 2.1 Hz, 1H), 6.19 (dd, J=17.0, 10.1 Hz, 1H), 6.33 (dd, J=17.0, 2.1 Hz, 1H), 6.43 (d, J=4.8 Hz, 1H), 6.67 (s, 1H), 6.70 (d, J=2.0 Hz, 1H), 6.87 (d, J=8.0 Hz, 1H), 7.16 (s, 1H), 7.29 (s, 1H), 7.39 (s, 1H), 7.94 (d, J=4.8 Hz, 1H).

MP: 130° C. (Mettler Toledo MP50), uncorrected.

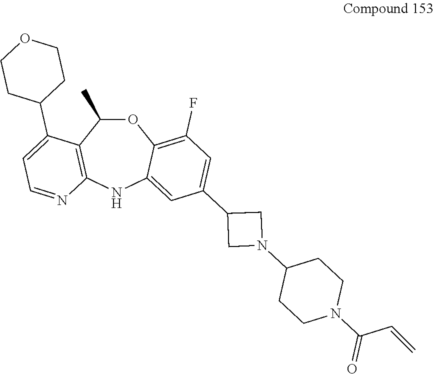 [SMILES: C=CC(=O)N1CCC(N2CC(c3cc(F)c4c(c3)Nc3nccc(C5CCOCC5)c3[C@@H](C)O4)C2)CC1]

(*R), pure stereoisomer but absolute stereochemistry undetermined Compound 153 was synthesized in a similar manner as Compound 263 using Intermediate 475 instead of Intermediate 57.

LC MS: confirms the MW (RT: 1.978, [M+H]⁺: 507.2, Method: 2).

¹H NMR: (400 MHz, Chloroform-d) δ (ppm) 0.81-0.92 (m, 2H), 1.23-1.38 (m, 2H), 1.41 (d, J=6.9 Hz, 3H), 1.68-1.83 (m, 3H), 1.83-1.97 (m, 1H), 2.29-2.40 (m, 1H), 2.98-3.29 (m, 5H), 3.48-3.62 (m, 3H), 3.69 (t, J=7.1 Hz, 2H), 3.88 (d, J=13.7 Hz, 1H), 4.05-4.16 (m, 2H), 4.24 (d, J=13.2 Hz, 1H), 5.67 (d, 1H), 5.91 (q, J=7.0 Hz, 1H), 6.24 (d, 1H), 6.47-6.52 (m, 1H), 6.55 (d, J=10.5 Hz, 1H), 6.58-6.66 (m, 1H), 6.70 (d, J=5.3 Hz, 1H), 7.19 (s, 1H), 8.07 (d, J=5.3 Hz, 1H).

MP: 136.4° C. (Mettler Toledo MP50), uncorrected.

OR: −8.84° (589 nm, c 0.173 w/v, MeOH, 23° C.).

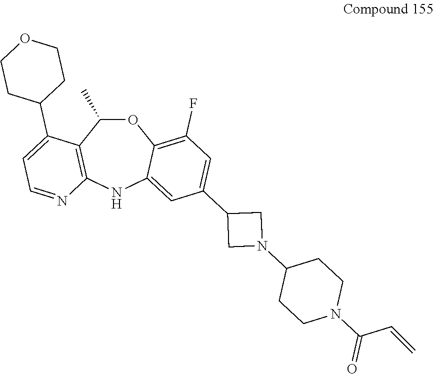 [SMILES: C=CC(=O)N1CCC(N2CC(c3cc(F)c4c(c3)Nc3nccc(C5CCOCC5)c3[C@H](C)O4)C2)CC1]

(*S), pure stereoisomer but absolute stereochemistry undetermined Compound 155 was synthesized in a similar manner as Compound 263 using Intermediate 476 instead of Intermediate 57.

LC MS: confirms the MW (RT: 1.969, [M+H]⁺: 507.2, Method: 2).

¹H NMR: (400 MHz, Chloroform-d) δ (ppm) 0.81-0.92 (m, 2H), 1.23-1.38 (m, 2H), 1.41 (d, J=6.9 Hz, 3H), 1.68-1.83 (m, 3H), 1.83-1.97 (m, 1H), 2.29-2.40 (m, 1H), 2.98-3.29 (m, 5H), 3.48-3.62 (m, 3H), 3.69 (t, J=7.1 Hz, 2H), 3.88 (d, J=13.7 Hz, 1H), 4.05-4.16 (m, 2H), 4.24 (d, J=13.2 Hz, 1H), 5.67 (d, 1H), 5.91 (q, J=7.0 Hz, 1H), 6.24 (d, 1H), 6.47-6.52 (m, 1H), 6.55 (d, J=10.5 Hz, 1H), 6.58-6.66 (m, 1H), 6.70 (d, J=5.3 Hz, 1H), 7.19 (s, 1H), 8.07 (d, J=5.3 Hz, 1H).

MP: 129.7° C. (Mettler Toledo MP50), uncorrected.

OR: +8.58° (589 nm, c 0.186 w/v, MeOH, 23° C.).

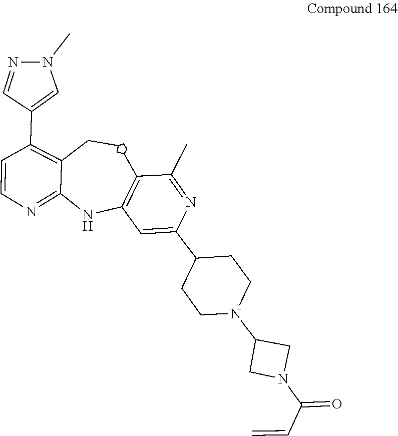 [SMILES: C=CC(=O)N1CC(N2CCC(c3cc4c(c(C)n3)C3CCC(Cc5c(-c6cnn(C)c6)ccnc5N4)C3)CC2)C1]

Intermediate 50 (341 mg, 0.79 mmol) was dissolved in a mixture of Et₃N (220 μL, 1.58 mmol, 2 eq.) and DCM (10 mL). The mixture was cooled in an ice-water bath. Acryloyl chloride (77 μL, 0.948 mmol, 1.2 eq.) was then added and the reaction mixture was stirred at 0° C. for 30 min The reaction mixture was concentrated to dryness. The residue was dissolved in DCM and washed with a mixture of Na₂CO₃ (1 M in water, 10 mL) and brine (5 mL). The organic layer was dried over MgSO₄, filtered, and concentrated to dryness. The residue was purified by reverse phase chromatography (Phenomenex Gemini C18 30×100 mm 5 μm, gradient from 81% H₂O−19% ACN-MeOH to 45% H₂O−55% ACN-MeOH [25 mM NH₄HCO₃]. The desired fractions were triturated in Et₂O, filtered, and dried to afford Compound 164 (172 mg, yield: 44%).

LCMS: confirms the MW (RT: 1.47, [M+H]⁺ 486, Method: 2).

MP: 277.8° C. (Mettler Toledo FP62) 10° C./min, uncorrected.

¹H NMR (300 MHz, CDCl₃) δ (ppm) 8.10 (d, J=4.9 Hz, 1H), 7.59 (s, 1H), 7.52 (s, 1H), 7.23 (s, 1H), 6.78 (d, J=4.9 Hz, 1H), 6.47 (s, 1H), 6.33 (d, J=16.8 Hz, 1H), 6.19 (dd, J=17.0, 10.1 Hz, 1H), 5.66 (d, J=10.1 Hz, 1H), 5.12 (s, 2H), 4.23 (t, J=7.7 Hz, 1H), 4.18-4.06 (m, 2H), 4.01 (s, 3H), 3.99-3.90 (m, 1H), 3.28-3.14 (m, 1H), 2.94 (t, J=12.7 Hz, 2H), 2.61 (t, J=11.9 Hz, 1H), 2.44 (s, 3H), 1.98 (s, 2H), 1.86-1.66 (m, 4H)

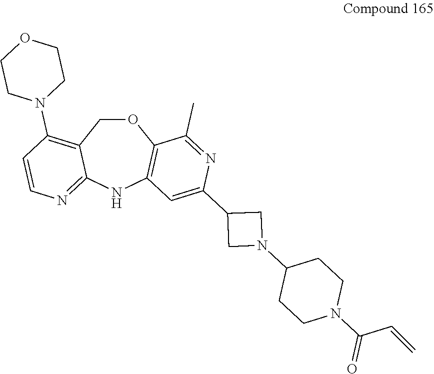 [SMILES: C=CC(=O)N1CCC(N2CC(c3cc4c(c(C)n3)OCc3c(N5CCOCC5)ccnc3N4)C2)CC1]

Compound 165 was synthesized in a similar manner as Compound 173 using Intermediate 479 instead of Intermediate 65.

LC MS: confirms the MW (RT: 1.453, [M/2+H]⁺: 246.3284, Method: 3).

¹H NMR (300 MHz, DMSO-d₆) δ (ppm) 9.44 (s, 1H), 8.04 (d, J=5.4 Hz, 1H), 7.01 (s, 1H), 6.79 (dd, J=16.7, 10.5 Hz, 1H), 6.56 (d, J=5.4 Hz, 1H), 6.06 (d, J=16.7 Hz, 1H), 5.64 (d, J=10.6 Hz, 1H), 5.01 (s, 2H), 4.04-3.92 (m, 1H), 3.90-3.80 (m, 1H), 3.79-3.72 (m, 4H), 3.54-3.39 (m, 3H), 3.29-2.97 (m, 4H), 2.95-2.86 (m, 4H), 2.33-2.25 (m, 1H), 2.32 (s, 3H), 1.72-1.56 (m, 2H), 1.21-1.01 (m, 2H)

MP: 197.5° C. (Mettler Toledo FP62), 10° C./min; uncorrected.

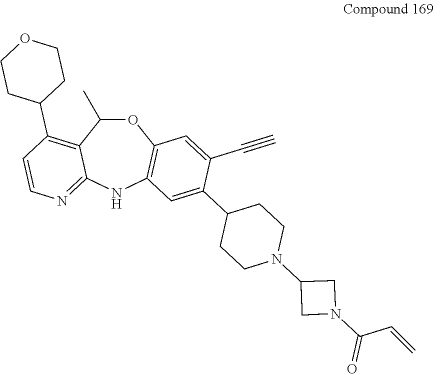 [SMILES: C#Cc1cc2c(cc1C1CCN(C3CN(C(=O)C=C)C3)CC1)Nc1nccc(C3CCOCC3)c1C(C)O2]

Compound 169 was prepared using a procedure analogous to Compound 263, starting from Intermediate 489 instead of Intermediate 57.

LC MS: confirms the MW (RT: 2.17, [M−H⁺]⁻: 512.2787, Method: 8).

¹H NMR: (500 MHz, Chloroform-d) δ (ppm) 1.38 (d, J=6.94 Hz, 3H) 1.99-2.08 (m, 2H) 2.95 (br d, J=10.40 Hz, 1H) 2.98-3.09 (m, 3H) 3.21 (s, 1H) 3.22-3.29 (m, 1H) 3.52-3.62 (m, 2H) 3.95-4.05 (m, 1H) 4.05-4.20 (m, 4H) 4.21-4.30 (m, 1H) 5.68 (dd, J=10.26, 1.01 Hz, 1H) 5.78 (q, J=6.94 Hz, 1H) 6.16-6.27 (m, 1H) 6.29-6.39 (m, 1H) 6.62 (s, 1H) 6.69 (d, J=5.20 Hz, 1H) 7.09 (s, 1H) 7.20 (s, 1H) 8.07 (d, J=5.20 Hz, 1H)

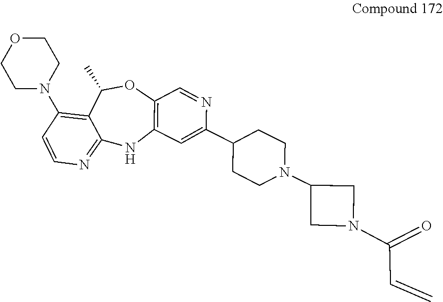 [SMILES: C=CC(=O)N1CC(N2CCC(c3cc4c(cn3)O[C@@H](C)c3c(N5CCOCC5)ccnc3N4)CC2)C1]

(*S), pure stereoisomer but absolute stereochemistry undetermined Compound 172 was prepared using a procedure analogous to Compound 263, starting from Intermediate 58 instead of Intermediate 57.

LCMS: confirms the MW (RT: 1.44 [M+H]⁺ 491, Method: 2).

MP: 271.9° C. (Mettler Toledo MP50), uncorrected.

OR: −87° (589 nm, c 0.733 w/v, MeOH, 23° C.).

¹H NMR (300 MHz, Chloroform-d) d (ppm) 1.40 (d, J=6.9 Hz, 3H), 1.67-1.85 (in. 2H), 1.90-2.06 (m, 4H), 2.64 (tt, J=12.1, 3.5 Hz, 1H), 2.84-3.03 (m, 4H), 3.04-3.16 (m, 2H), 3.21 (p, J=6.4 Hz, 1H), 3.80-3.91 (m, 4H), 3.97 (dd, J=10.7, 5.7 Hz, 1H), 4.05-4.16 (m, 2H), 4.23 (t. J=7.9 Hz, 1H), 5.66 (dd, J=10.1, 2.1 Hz. 1H), 5.86 (t, J=6.9 Hz, 1H), 6.19 (dd, J=17.0, 10.1 Hz, 1H), 6.33 (dd, J=17.0, 2.1 Hz, 1H), 6.53 (s, 1H), 6.55 (s, 1H), 7.39 (s, 1H), 8.06 (d, J=5.5 Hz, 1H), 8.09 (s, 1H).

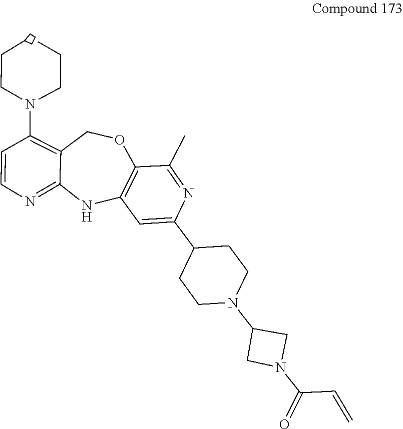 [SMILES: C=CC(=O)N1CC(N2CCC(c3cc4c(c(C)n3)OCc3c(N(CCC)CCC5CCC5)ccnc3N4)CC2)C1]

Intermediate 65 (216 mg, 0.495 mmol) was dissolved in a mixture of Et₃N (138 μL, 0.99 mmol, 2 eq.) and DCM (15 mL). The mixture was cooled to 0° C. and acryloyl chloride (52 μL, 0.643 mmol, 1.3 eq.) was added. The reaction mixture was stirred at 0° C. for 30 min. The volatiles were evaporated, the residue was dissolved in DCM and this solution was washed with Na₂CO₃ (1 M in water, 10 mL). The organic layer was dried over MgSO₄, filtered, and evaporated. The residue was purified by column chromatography over silica gel (DCM/MeOH 9/1 in DCM from 0 to 100%) followed by reverse phase column chromatography (Phenomenex Gemini C18 100×30 mm 5 μm; gradient from 72% H₂O−28% ACN/MeOH to 36% H₂O−74% ACN/MeOH, 25 mM NH₄HCO₃). The desired fractions were combined and the product was triturated in Et₂O to give Compound 173 (18 mg, yield: 7%) as a white solid.

LCMS: confirms the MW (RT: 1.46 [M+H]⁺ 491, Method: 2).

MP: 236.9° C. (Mettler Toledo FP 62), 10° C./min, uncorrected.

¹H NMR (300 MHz, CDCl₃) δ (ppm) 8.04 (d, J=5.5 Hz, 1H), 7.32 (s, 1H), 6.48 (d, J=5.4 Hz, 1H), 6.47 (s, 1H), 6.33 (d, J=16.9 Hz, 1H), 6.19 (dd, J=17.0, 10.1 Hz, 1H), 5.66 (d, J=10.2 Hz, 1H), 5.05 (s, 2H), 4.23 (t, J=7.8 Hz, 1H), 4.17-4.05 (m, 2H), 4.00-3.92 (m, 1H), 3.92-3.85 (m, 4H), 3.27-3.13 (m, 1H), 3.10-3.00 (m, 4H), 2.94 (t, J=13.0 Hz, 2H), 2.65 (t, J=11.9 Hz, 1H), 2.46 (s, 3H), 2.06-1.92 (m, 4H), 1.73 (t, J=11.8 Hz, 2H)

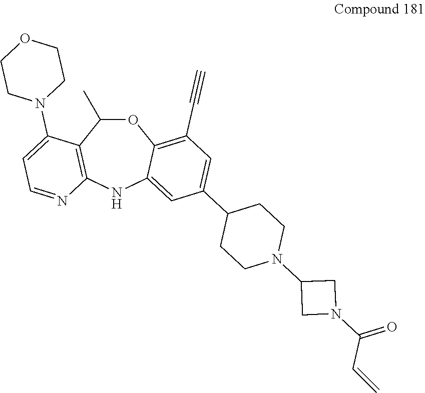 [SMILES: C#Cc1cc(C2CCN(C3CN(C(=O)C=C)C3)CC2)cc2c1OC(C)c1c(N3CCOCC3)ccnc1N2]

Compound 181 was synthesized in a similar manner as Intermediate 184 using Intermediate 498 instead of Intermediate 183.

LC MS: confirms the MW (RT: 2.14, [M+H]⁺⁻: 514.2, Method: 13).

¹H NMR: (400 MHz, Chloroform-d) δ (ppm) 1.41 (d, J=6.9 Hz, 3H) 1.70-1.80 (m, 2H) 1.88 (br d, J=12.7 Hz, 2H) 1.93-2.04 (m, 2H) 2.38-2.51 (m, 1H) 2.85-3.03 (m, 6H) 3.17-3.25 (m, 1H) 3.26 (s, 1H) 3.86-3.92 (m, 4H) 3.98 (dd, J=10.4, 5.1 Hz, 1 H) 4.09-4.17 (m, 2H) 4.22-4.30 (m, 1H) 5.68 (dd, J=10.2, 2.1 Hz, 1H) 5.99-6.09 (m, 1H) 6.15-6.27 (m, 1H) 6.30-6.39 (m, 1H) 6.52 (d, J=5.5 Hz, 1H) 6.62 (d, J=1.8 Hz, 1H) 6.91 (d, J=1.8 Hz, 1H) 7.03 (s, 1H) 8.04 (d, J=5.3 Hz, 1H).

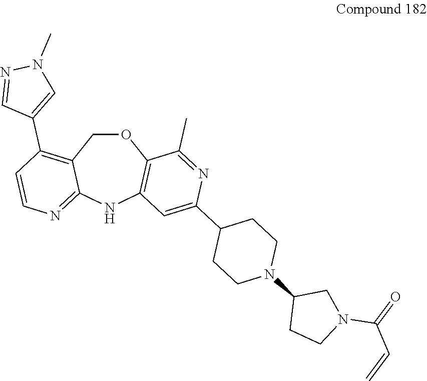 [SMILES: C=CC(=O)N1CC[C@@H](N2CCC(c3cc4c(c(C)n3)OCc3c(-c5cnn(C)c5)ccnc3N4)CC2)C1]

(*R), pure stereoisomer but absolute stereochemistry undetermined Compound 182 was synthesized in a simuilar manner as Compound 173 using Intermediate 501 instead of Intermediate 65.

LC MS: confirms the MW (RT: 1.439, [M+H]⁺: 500, Method: 2).

¹H NMR (300 MHz, CDCl₃) δ (ppm) 8.11 (d, J=5.1 Hz, 1H), 7.59 (s, 1H), 7.52 (s, 1H), 7.19 (br s, 1H), 6.78 (d, J=5.0 Hz, 1H), 6.52-6.36 (m, 3H), 5.67 (d, J=8.5 Hz, 1H), 5.13 (s, 2H), 4.11-3.94 (m, 0.5H), 4.01 (s, 3H), 3.92-3.69 (m, 1.5H), 3.58-3.27 (m 2H), 3.22-2.76 (m, 3H), 2.64 (s, 1H), 2.45 (s, 3H), 2.32-2.11 (m, 3H), 2.00-1.90 (m, 3H), 1.88-1.66 (m, 3H)

MP: 179.4° C. (Mettler Toledo FP62), 10° C./min. uncorrected.

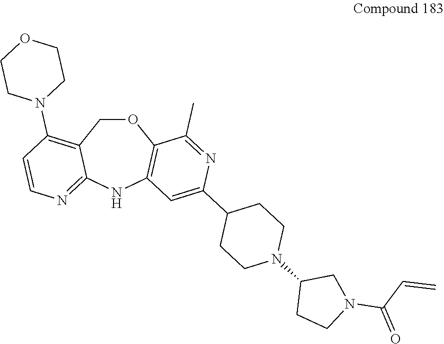 [SMILES: C=CC(=O)N1CC[C@H](N2CCC(c3cc4c(c(C)n3)OCc3c(N5CCOCC5)ccnc3N4)CC2)C1]

(*S), pure stereoisomer but absolute stereochemistry undetermined Compound 183 was synthesised in a similar manner as Compound 164 using Intermediate 504 instead of Intermediate 50.

LC MS: confirms the MW (RT: 1.420, [M+H]⁺: 505.2, Method: 2).

¹H NMR (300 MHz, CDCl₃) δ (ppm) 8.03 (d, J=5.3 Hz, 1H), 7.23 (br s, 1H), 6.47-6.33 (m, 4H), 5.67 (d, J=8.7 Hz, 1H), 5.05 (s, 2H), 4.08-3.69 (m, 5H), 3.60-3.22 (m, 2H), 3.12 (d, J=9.5 Hz, 1H), 3.08-2.74 (m, 6H), 2.59 (t, J=11.2 Hz, 1H), 2.45 (s, 3H), 2.30-1.88 (m, 6H), 1.80-1.65 (m, 3H)

OR: −19.7304° (589 nm, c 0.2720 w/v, MeOH, 23.0° C.).

MP: 201.7° C. (Mettler Toledo FP62), 10° C., uncorrected.

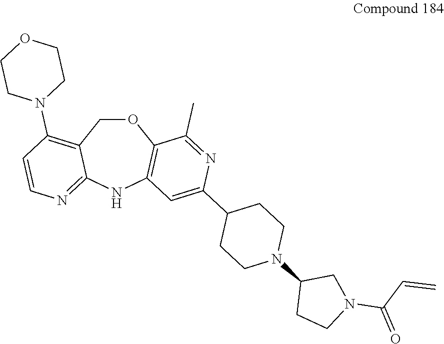 [SMILES: C=CC(=O)N1CC[C@@H](N2CCC(c3cc4c(c(C)n3)OCc3c(N5CCOCC5)ccnc3N4)CC2)C1]

(*R), pure stereoisomer but absolute stereochemistry undetermined Compound 184 was synthesised in a similar manner as Compound 164 using Intermediate 505 instead of Intermediate 50.

LC MS: confirms the MW (RT: 1.1413, [M+H]⁺: 505.2, Method: 2).

¹H NMR (300 MHz, CDCl₃) δ (ppm) 8.03 (d, J=5.3 Hz, 1H), 7.23 (br s, 1H), 6.47-6.33 (m, 4H), 5.67 (d, J=8.7 Hz, 1H), 5.05 (s, 2H), 4.08-3.69 (m, 5H), 3.60-3.22 (m, 2H), 3.12 (d, J=9.5 Hz, 1H), 3.08-2.74 (m, 6H), 2.59 (t, J=11.2 Hz, 1H), 2.45 (s, 3H), 2.30-1.88 (m, 6H), 1.80-1.65 (m, 3H)

MP: 202.2° C. (Mettler Toledo FP62), 10° C., uncorrected.

OR: +22.7093° (589 nm, c 0.20707 w/v, MeOH, 23.0° C.).

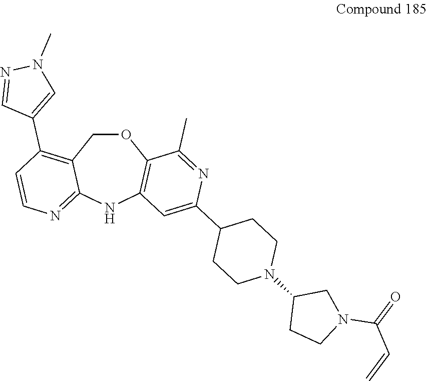 [SMILES: C=CC(=O)N1CC[C@H](N2CCC(c3cc4c(c(C)n3)OCc3c(-c5cnn(C)c5)ccnc3N4)CC2)C1]

(*S), pure stereoisomer but absolute stereochemistry undetermined Compound 185 was synthesized in a similar manner as Compound 173 using Intermediate 502 instead of Intermediate 65.

LC MS: confirms the MW (RT: 1.448, [M+H]⁺: 500, Method: 2).

¹H NMR (300 MHz, CDCl₃) δ (ppm) 8.11 (d, J=5.1 Hz, 1H), 7.59 (s, 1H), 7.52 (s, 1H), 7.19 (br s, 1H), 6.78 (d, J=5.0 Hz, 1H), 6.52-6.36 (m, 3H), 5.67 (d, J=8.5 Hz, 1H), 5.13 (s, 2H), 4.11-3.94 (m, 0.5H), 4.01 (s, 3H), 3.92-3.69 (m, 1.5H), 3.58-3.27 (m 2H), 3.22-2.76 (m, 3H), 2.64 (s, 1H), 2.45 (s, 3H), 2.32-2.11 (m, 3H), 2.00-1.90 (m, 3H), 1.88-1.66 (m, 3H)

MP: 186.6° C. (Mettler Toledo FP62), 10° C./min, uncorrected.

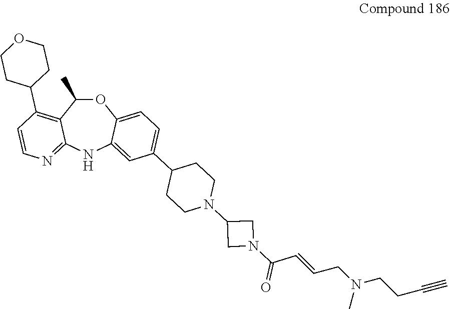 [SMILES: C#CCCN(C)C/C=C/C(=O)N1CC(N2CCC(c3ccc4c(c3)Nc3nccc(C5CCOCC5)c3[C@@H](C)O4)CC2)C1]

(*R), pure stereoisomer but absolute stereochemistry undetermined Compound 186 was synthesized in a similar manner as Compound 195 using Intermediate 401 instead of Intermediate 388.

LC MS: confirms the MW (RT: 1.958, [M+H]⁺: 584.2, Method: 12).

¹H NMR (400 MHz, DMSO-d₆) δ (ppm) 9.11 (s, 1H), 7.99 (d, J=5.1 Hz, 1H), 7.04 (d, J=1.8 Hz, 1H), 6.76 (d, J=8.2 Hz, 1H), 6.66 (d, J=5.3 Hz, 1H), 6.62-6.48 (m, 2H), 6.11 (d, J=15.4 Hz, 1H), 5.78 (q, J=6.6 Hz, 1H), 4.19 (t, J=7.8 Hz, 1H), 4.02-3.85 (m, 4H), 3.70 (dd, J=5.1, 10.4 Hz, 1H), 3.57-3.44 (m, 2H), 3.16-3.05 (m, 4H), 2.86 (br t, J=12.0 Hz, 2H), 2.76 (t, J=2.6 Hz, 1H), 2.44 (s, 2H), 2.41-2.22 (m, 3H), 2.15 (s, 3H), 1.86 (br t, J=11.9 Hz, 2H), 1.76-1.45 (m, 8H), 1.20 (d, J=6.8 Hz, 3H).

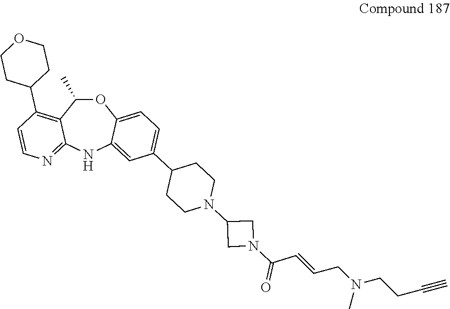 [SMILES: C#CCCN(C)C/C=C/C(=O)N1CC(N2CCC(c3ccc4c(c3)Nc3nccc(C5CCOCC5)c3[C@H](C)O4)CC2)C1]

(*S), pure stereoisomer but absolute stereochemistry undetermined Compound 187 was synthesized in a similar manner as Compound 195 using Intermediate 402 instead of Intermediate 388.

LC MS: confirms the MW (RT: 1.995, [M+H]⁺: 584.2, Method: 12).

¹H NMR (400 MHz, DMSO-d₆) δ (ppm) 9.12 (s, 1H), 7.99 (d, J=5.3 Hz, 1H), 7.04 (d, J=2.0 Hz, 1H), 6.76 (d, J=8.2 Hz, 1H), 6.67 (d, J=5.3 Hz, 1H), 6.63-6.50 (m, 2H), 6.11 (d, J=15.4 Hz, 1H), 5.78 (q, J=6.7 Hz, 1H), 4.19 (t, J=7.7 Hz, 1H), 4.03-3.86 (m, 4H), 3.70 (dd, J=5.4, 9.8 Hz, 1H), 3.56-3.43 (m, 2H), 3.11 (br d, J=6.0 Hz, 4H), 2.87 (br t, J=12.5 Hz, 2H), 2.77 (t, J=2.6 Hz, 1H), 2.44 (s, 2H), 2.37-2.23 (m, 3H), 2.15 (s, 3H), 1.93-1.81 (m, 2H), 1.75-1.47 (m, 8H), 1.20 (d, J=6.8 Hz, 3H).

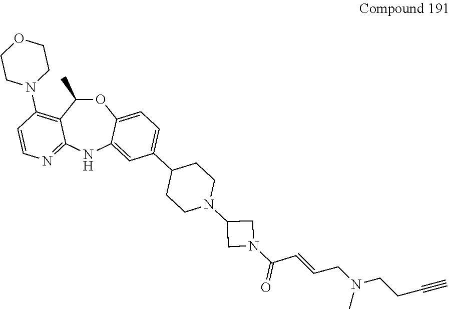 [SMILES: C#CCCN(C)C/C=C/C(=O)N1CC(N2CCC(c3ccc4c(c3)Nc3nccc(N5CCOCC5)c3[C@@H](C)O4)CC2)C1]

(*R), pure stereoisomer but absolute stereochemistry undetermined Compound 191 was synthesized in a similar manner as Compound 195 using Intermediate 387 instead of Intermediate 388.

LC MS: confirms the MW (RT: 1.953, [M+H]⁺: 585.2, Method: 12).

¹H NMR (400 MHz, DMSO-d₆) δ (ppm) 9.07 (s, 1H), 7.97 (d, J=5.3 Hz, 1H), 7.04 (d, J=2.0 Hz, 1H), 6.75 (d, J=8.2 Hz, 1H), 6.61-6.48 (m, 3H), 6.11 (d, J=15.4 Hz, 1H), 5.73 (q, J=6.8 Hz, 1H), 4.19 (t, J=8.0 Hz, 1H), 3.98 (br dd, J=4.7, 8.5 Hz, 1H), 3.93-3.84 (m, 1H), 3.76-3.65 (m, 5H), 3.14-3.07 (m, 3H), 2.95-2.74 (m, 7H), 2.44 (s, 2H), 2.37-2.23 (m, 3H), 2.15 (s, 3H), 1.86 (br t, J=11.6 Hz, 2H), 1.72 (br d, J=12.6 Hz, 2H), 1.55 (q, J=11.3 Hz, 2H), 1.24 (d, J=6.8 Hz, 3H).

### Compound 192 and Compound 194

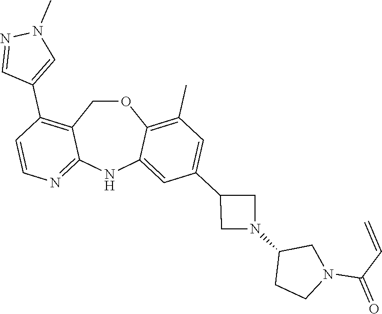 [SMILES: C=CC(=O)N1CC[C@H](N2CC(c3cc(C)c4c(c3)Nc3nccc(-c5cnn(C)c5)c3CO4)C2)C1] [1577] (*S), pure stereoisomer but absolute stereochemistry undetermined

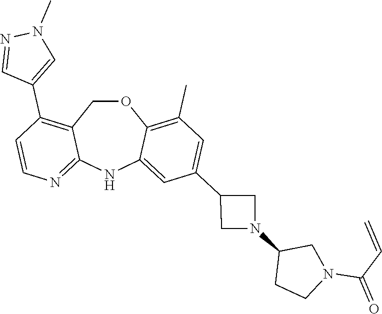 [SMILES: C=CC(=O)N1CC[C@@H](N2CC(c3cc(C)c4c(c3)Nc3nccc(-c5cnn(C)c5)c3CO4)C2)C1]

(*R), pure stereoisomer but absolute stereochemistry undetermined

Compound 192 and Compound 194 were synthesized in a similar manner as Compound 27 using Intermediate 510 instead of Intermediate 222. Purification via chiral SFC (Stationary phase: CHIRALCEL OD-H 5 μm 250*30 mm, Mobile phase: 45% C02, 55% EtOH(+0.6% TEA)) afforded the 2 enantiomers.

LC MS (Compound 192): confirms the MW (RT: 2.28, [M+H]⁺: 471.4, Method: 1).

¹H NMR (Compound 192): (500 MHz, DMSO-d₆, 32° C.): δ (ppm) 9.02 (d, J=4.7 Hz, 1H), 8.02 (d, J=5.0 Hz, 1H), 7.95 (s, 1H), 7.62 (s, 1H), 7.05 (d, J=2.2 Hz, 1H), 6.69 (d, J=5.0 Hz, 1H), 6.53-6.60 (m, 2H), 6.12 (dt, J=16.8, 2.6 Hz, 1H), 5.64 (ddd, J=10.4, 6.3, 2.5 Hz, 1H), 5.06 (s, 2H), 3.91 (s, 3H), 3.49-3.62 (m, 4H), 3.33-3.47 (m, 2H), 3.22-3.28 (m, 1H), 2.95-3.06 (m, 3H), 2.15 (s, 3H), 1.64-1.92 ppm (m, 2H)

SFC (Compound 192): RT: 1.94, 99.8%, [M+H]⁺: 472, Method: 4.

OR (Compound 192): +5° (589 nm, c 0.24 w/v %, DMF, 20° C.).

LC MS (Compound 194): confirms the MW (RT: 2.28, [M+H]⁺: 471.4, Method: 1).

¹H NMR (Compound 194): (500 MHz, DMSO-d₆, 31° C.): δ (ppm) 9.02 (d, J=4.4 Hz, 1H), 8.02 (d, J=5.0 Hz, 1H), 7.95 (s, 1H), 7.62 (s, 1H), 7.05 (d, J=1.9 Hz, 1H), 6.69 (d, J=5.0 Hz, 1H), 6.52-6.62 (m, 2H), 6.14 (t, J=2.7 Hz, 1H), 5.64 (ddd, J=10.2, 6.4, 2.4 Hz, 1H), 5.06 (s, 2H), 3.91 (s, 3H), 3.49-3.62 (m, 4H), 3.33-3.47 (m, 2H), 3.17-3.27 (m, 1H), 2.93-3.07 (m, 3H), 2.15 (s, 3H), 1.64-1.91 ppm (m, 2H)

SFC (Compound 194): RT: 1.29, 100%, [M+H]⁺: 472, Method: 4.

OR (Compound 194): −15.71° (436 nm, c 0.28 w/v 00 DMF, 20° C.).

### Compound 193 and Compound 196

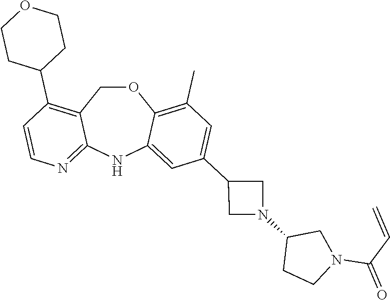 [SMILES: C=CC(=O)N1CC[C@H](N2CC(c3cc(C)c4c(c3)Nc3nccc(C5CCOCC5)c3CO4)C2)C1] [1589] Compound 193: (*S), pure stereoisomer but absolute stereochemistry undetermined

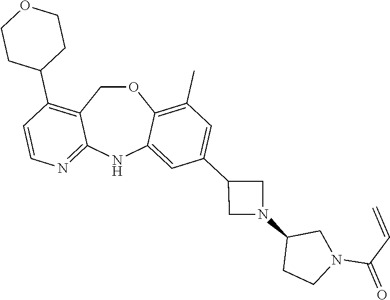 [SMILES: C=CC(=O)N1CC[C@@H](N2CC(c3cc(C)c4c(c3)Nc3nccc(C5CCOCC5)c3CO4)C2)C1]

Compound 196: (*R), pure stereoisomer but absolute stereochemistry undetermined

Compound 193 and Compound 196 were synthesized in a similar manner as Compound 27 using Intermediate 513 instead of Intermediate 222. Purification via chiral SFC (Stationary phase: Whelk-O1 (S,S) 5 μm 250*21.2 mm, Mobile phase: 45% CO2, 55% (MeOH/DCM:90/10)+(0.3% iPrNH2)) afforded the 2 enantiomers.

LC MS (Compound 193): confirms the MW (RT: 2.49, [M+H]⁺: 475.4, Method: 1).

¹H NMR (Compound 193): (400 MHz, DMSO-d₆, 23° C.): δ (ppm) 8.97 (d, J=3.7 Hz, 1H), 8.02 (d, J=5.4 Hz, 1H), 7.03 (d, J=2.0 Hz, 1H), 6.68 (d, J=5.4 Hz, 1H), 6.52-6.61 (m, 2H), 6.12 (dt, J=16.9, 2.2 Hz, 1H), 5.65 (ddd, J=10.3, 5.4, 2.4 Hz, 1H), 5.06 (s, 2H), 3.92 (br dd, J=10.5, 3.7 Hz, 2H), 3.38-3.62 (m, 8H), 3.11-3.31 (m, 3H), 2.94-3.07 (m, 3H), 2.17 (s, 3H), 1.52-1.91 (m, 6H), 1.14-1.32 ppm (m, 1H)

SFC (Compound 193): (RT: 2.89, 99.7%, [M+H]⁺: 475, Method: 3)

OR (Compound 193): +4.44° (589 nm, c 0.27 w/v %, DMF, 20° C.)

LC MS (Compound 196): confirms the MW (RT: 2.49, [M+H]⁺: 475.4, Method: 1).

¹H NMR (Compound 196): (400 MHz, DMSO-d₆, 22° C.): δ (ppm) 8.97 (d, J=3.9 Hz, 1H), 8.02 (d, J=5.1 Hz, 1H), 7.03 (d, J=2.0 Hz, 1H), 6.68 (d, J=5.4 Hz, 1H), 6.53-6.61 (m, 2H), 6.12 (dt, J=16.8, 2.2 Hz, 1H), 5.65 (ddd, J=10.2, 5.6, 2.6 Hz, 1H), 5.06 (s, 2H), 3.92 (br dd, J=10.8, 3.7 Hz, 2H), 3.35-3.61 (m, 8H), 3.24-3.27 (m, 1H), 3.11-3.19 (m, 1H), 2.95-3.05 (m, 3H), 2.16 (s, 3H), 1.54-1.93 ppm (m, 6H)

SFC (Compound 196): check EE purity (RT: 2.50, 100%, [M+H]⁺: 475, Method: 3)

OR (Compound 196): −4.83° (589 nm, c 0.29 w/v %, DMF, 20° C.)

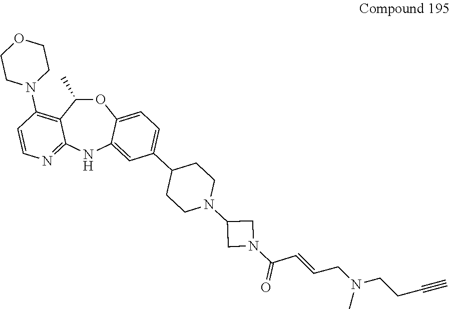 [SMILES: C#CCCN(C)C/C=C/C(=O)N1CC(N2CCC(c3ccc4c(c3)Nc3nccc(N5CCOCC5)c3[C@H](C)O4)CC2)C1]

(*S), pure stereoisomer but absolute stereochemistry undetermined

HATU (236 mg, 0.62 mmol, 1.5 eq.) was added to the mixture of Intermediate 388 (180 mg, 0.41 mmol, 1.0 eq.), 4-(but-3-yn-1-yl(methyl)amino)but-2-enoic acid ([CAS: 2165285-45-8], 69 mg, 0.41 mmol, 1.0 eq.), DIPEA (160 mg, 1.2 mmol, 3.0 eq.) in DMF (5 mL). The reaction mixture was stirred at room temperature overnight. The reaction mixture was diluted with water and extracted with DCM. The organic layer was dried on MgSO₄, filtered, and evaporated. The residue was purified by preparative high-performance liquid chromatography (Column: Waters Xbridge, 150*25 mm 5 μm, Condition: A: water (0.05% NH₄OH v/v), B: ACN; A/B from 60/40 to 40/60). A second purification was performed with Supercritical Fluid Chromatography (Column: Daicel Chiralcel OD (250 mm*30 mm, 10 μm; mobile phase: A: 0.1% NH₃ H₂O, B: EtOH (0.1% NH₃ H₂O), A:B=50:50) to afford Compound 195 (8 mg, yield: 3%) as white solid.

LC MS: confirms the MW (RT: 1.964, [M+H]⁺: 585.2, Method: 12).

¹H NMR (400 MHz, DMSO-d₆) δ (ppm) 9.06 (s, 1H), 7.97 (d, J=5.5 Hz, 1H), 7.04 (d, J=1.8 Hz, 1H), 6.74 (d, J=8.2 Hz, 1H), 6.60-6.48 (m, 3H), 6.11 (d, J=15.4 Hz, 1H), 5.72 (q, J=6.7 Hz, 1H), 4.18 (t, J=8.0 Hz, 1H), 3.98 (br dd, J=5.2, 8.7 Hz, 1H), 3.89 (dd, J=7.6, 9.8 Hz, 1H), 3.78-3.66 (m, 5H), 3.15-3.05 (m, 3H), 2.94-2.74 (m, 7H), 2.46-2.43 (m, 2H), 2.38-2.24 (m, 3H), 2.14 (s, 3H), 1.85 (br t, J=11.5 Hz, 2H), 1.71 (br d, J=11.5 Hz, 2H), 1.63-1.48 (m, 2H), 1.27 (br s, 1H), 1.24 (d, J=6.8 Hz, 2H).

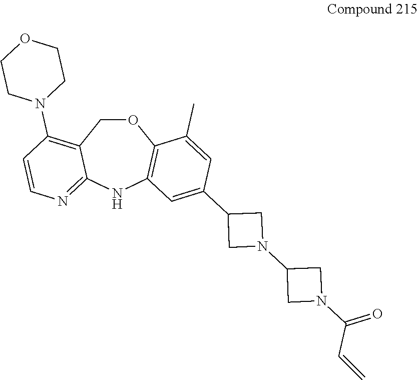 [SMILES: C=CC(=O)N1CC(N2CC(c3cc(C)c4c(c3)Nc3nccc(N5CCOCC5)c3CO4)C2)C1]

NaOtBu [CAS: 865-48-5] (0.74 mL, 2 M, 1.48 mmol) was added to Intermediate 516 (0.74 g, 1.366 mmol) in THF (8 mL) at −15° C. and the reaction mixture was stirred at −15° C. for 30 min. Water, NH₄Cl saturated and DCM were added, the mixture was extracted, the organic layer was decanted with chromabondR PTS and the solvent was evaporated until dryness The residue was purified via column chromatography (Stationary phase: irregular SiOH 15-40 μm 40 g, Mobile phase: gradient from 100% DCM to 90% DCM, 10% MeOH, 1% NH₄OH). This product was taken up into ACN, triturated and cristallized overnight. This product was filtered and dried until dryness to give Compound 215 (58 mg, yield: 9%).

LC MS: confirms the MW (RT: 2.40, [M+H]⁺: 462.3, Method: 1).

¹H NMR: NMR (500 MHz, DMSO-d₆, 21° C.): δ (ppm) 8.94 (s, 1H), 7.97 (d, J=5.4 Hz, 1H), 7.03 (d, J=2.2 Hz, 1H), 6.60 (d, J=2.2 Hz, 1H), 6.44 (d, J=5.4 Hz, 1H), 6.32 (dd, J=17.0, 10.4 Hz, 1H), 6.09 (dd, J=17.0, 2.2 Hz, 1H), 5.66 (dd, J=10.1, 2.2 Hz, 1H), 4.98 (s, 2H), 4.22 (t, J=8.0 Hz, 1H), 3.87-4.04 (m, 2H), 3.67-3.81 (m, 5H), 3.54-3.62 (m, 2H), 3.40-3.51 (m, 2H), 3.09 (q, J=7.4 Hz, 2H), 2.82-2.92 (m, 4H), 2.17 (s, 3H), 2.08 ppm (s, 2H).

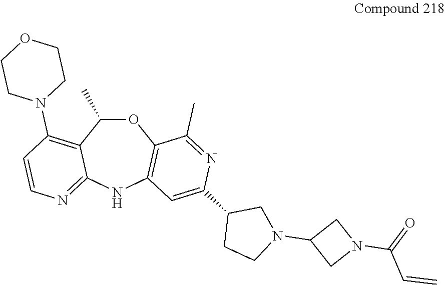 [SMILES: C=CC(=O)N1CC(N2CC[C@H](c3cc4c(c(C)n3)O[C@@H](C)c3c(N5CCOCC5)ccnc3N4)C2)C1]

Compound 218 (*S, *S), pure isomer, absolute stereochemistry undetermined Compound 218 was synthesized following a similar sequence of reactions as for the synthesis of Compound 245 starting from Intermediate 555C instead of Intermediate 555A.

LC MS: confirms the MW (RT: 1.546, [M+H]⁺: 491, Method: 2)

¹H NMR (300 MHz, Chloroform-d) d (ppm) 1.35 (d, J=6.9 Hz, 3H), 1.96-2.14 (m, 1H), 2.24-2.50 (m, 2H), 2.65-2.81 (m, 3H), 2.87-2.99 (m, 2H), 2.99-3.13 (m, 2H), 3.35-3.53 (m, 3H), 3.80-3.97 (m, 4H), 3.97-4.08 (m, 1H), 4.08-4.20 (m, 2H), 4.26 (t, J=8.0 Hz, 1H), 5.66 (d, J=9.9 Hz, 1H), 5.96 (q, J=6.9 Hz, 1H), 6.23 (d, J=10.1 Hz, 1H), 6.33 (d, J=16.8 Hz, 1H), 6.54 (t, J=7.1 Hz, 2H), 7.16 (s, 1H), 8.06 (d, J=5.6 Hz, 1H).

MP: >300° C. (Mettler Toledo MP50), uncorrected.

OR: −628748° (589 nm, c 0.1067 w/v, MeOH).

### Compound 222 and Compound 223

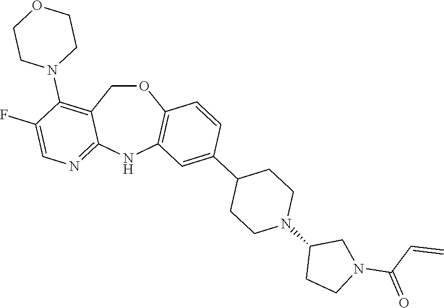 [SMILES: C=CC(=O)N1CC[C@H](N2CCC(c3ccc4c(c3)Nc3ncc(F)c(N5CCOCC5)c3CO4)CC2)C1] [1613] Compound 222: (*S), pure stereoisomer but absolute stereochemistry undetermined

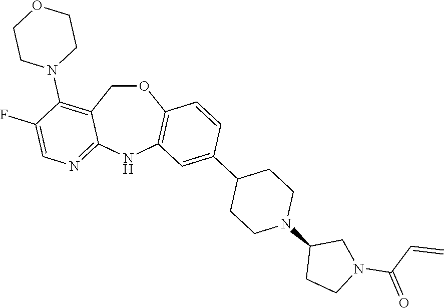 [SMILES: C=CC(=O)N1CC[C@@H](N2CCC(c3ccc4c(c3)Nc3ncc(F)c(N5CCOCC5)c3CO4)CC2)C1]

Compound 223: (*R), pure stereoisomer but absolute stereochemistry undetermined

Acrylic acid (126 μL, 1.8 mmol, 1.0 eq.) in DCM (5 mL) was added dropwise to a solution of Intermediate 517 (900 mg, 1.8 mmol, 1.0 eq.), EDCI HCl (422.5 mg, 2.2 mmol, 1.2 eq.), 1-hydroxybenzotriazole ([CAS: 2592-95-2], 297 mg, 2.2 mmol, 1.2 eq.), Et₃N (1.0 mL, 7.3 mmol, 4.0 eq.) in DCM (5 mL) at 5° C. and the reaction mixture was stirred at room temperature overnight. H₂O and DCM were added and the reaction mixture was extracted. The organic layer was separated, dried over MgSO₄, filtered, and evaporated. A purification was performed via column chromatography (Stationary phase: irregular SiOH 40 μm 25 g, Mobile phase: 97/3/0.1 to 90/10/0.1 CMA). The isomers were separated with chiral SFC (Stationary phase: Whelk-O1 (S,S) 5 μm 250*21.2 mm, Mobile phase: 40% CO₂, 60% MeOH (0.6% Et₃N)) yielding Compound 222 (71 mg, yield: 8%) and Compound 223 (58 mg, yield: 6%).

¹H NMR (Compound 222): (500 MHz, DMSO-d₆, 22° C.): δ (ppm) 8.96 (d, J=2.2 Hz, 1H), 8.00 (d, J=4.1 Hz, 1H), 7.06 (d, J=1.9 Hz, 1H), 6.81 (d, J=8.2 Hz, 1H), 6.53-6.66 (m, 2H), 6.08-6.21 (m, 1H), 5.65 (ddd, J=10.3, 7.8, 2.4 Hz, 1H), 5.03 (s, 2H), 3.69-3.92 (m, 5H), 3.36-3.62 (m, 2H), 3.16-3.30 (m, 1H), 3.00-3.15 (m, 5H), 2.73-2.97 (m, 2H), 2.30-2.40 (m, 1H), 2.01-2.18 (m, 3H), 1.52-1.83 ppm (m, 5H)

m.p. (Kofler): 118° C.

LC MS (Compound 223): confirms the MW (RT: 2.63, [M+H]⁺: 508.4, Method: 1).

¹H NMR (Compound 223): (500 MHz, DMSO-d₆, 22° C.): δ (ppm) 8.96 (d, J=2.2 Hz, 1H), 8.00 (d, J=3.8 Hz, 1H), 7.06 (d, J=1.9 Hz, 1H), 6.81 (d, J=8.2 Hz, 1H), 6.53-6.67 (m, 2H), 6.09-6.24 (m, 1H), 5.66 (ddd, J=10.2, 7.6, 2.4 Hz, 1H), 5.03 (s, 2H), 3.68-3.93 (m, 5H), 3.37-3.63 (m, 2H), 3.17-3.30 (m, 1H), 3.07-3.15 (m, 3H), 3.00-3.07 (m, 1H), 2.74-2.96 (m, 2H), 2.25-2.40 (m, 1H), 2.01-2.18 (m, 3H), 1.55-1.81 ppm (m, 4H)

OR (Compound 223): +7.81° (589 nm, c 0.32 w/v %, DMF, 20° C.).

m.p. (Kofler): 106° C.

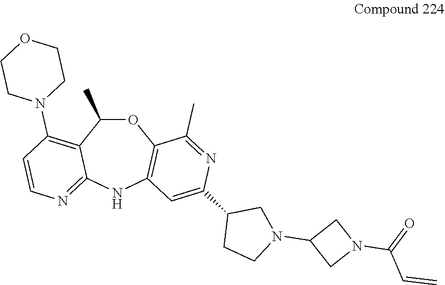 [SMILES: C=CC(=O)N1CC(N2CC[C@H](c3cc4c(c(C)n3)O[C@H](C)c3c(N5CCOCC5)ccnc3N4)C2)C1]

Compound 224 (*R, *S), pure isomer, absolute stereochemistry undetermined

Compound 224 was synthesized following a similar sequence of reactions as for the synthesis of Compound 245 starting from Intermediate 555D instead of Intermediate 555A.

LC MS: confirms the MW (RT: 1.556, [M+H]⁺: 491, Method: 2)

¹H NMR (300 MHz, Chloroform-d) d (ppm) 1.36 (d, J=6.9 Hz, 3H), 1.86-2.24 (m, 1H), 2.40 (d, J=9.3 Hz, 2H), 2.49 (s, 3H), 2.82 (s, 2H), 2.84-2.98 (m, 2H), 3.07 (dt, J=9.6, 4.4 Hz, 2H), 3.50 (s, 3H), 3.83-3.95 (m, 4H), 4.06 (s, 1H), 4.10-4.34 (m, 3H), 5.67 (dd, J=10.1, 2.1 Hz, 1H), 5.97 (q, J=6.9 Hz, 1H), 6.14-6.26 (m, 1H), 6.31 (d, J=2.1 Hz, 1H), 6.37 (d, J=2.2 Hz, 1H), 6.59 (d, J=5.7 Hz, 2H), 8.08 (d, J=5.6 Hz, 1H).

MP: 148° C. (Mettler Toledo MP50), uncorrected.

OR: −17.25° (589 nm, c 0.08 w/v, MeOH. 23° C.).

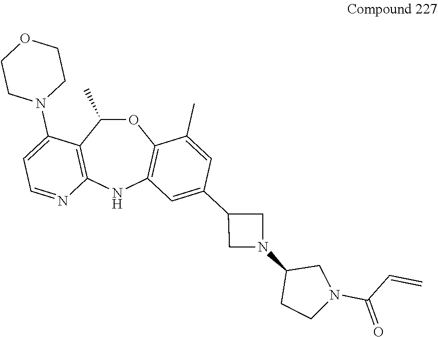 [SMILES: C=CC(=O)N1CC[C@@H](N2CC(c3cc(C)c4c(c3)Nc3nccc(N5CCOCC5)c3[C@H](C)O4)C2)C1]

(7*S, 20*R), pure isomer but absolute stereochemistry undetermined

Compound 227 was synthesized in a similar manner as Compound 29 using Intermediate 524 instead of Intermediate 229.

LC MS: confirms the MW (RT: 2.802, [M+H]⁺: 490.2, Method: 14).

¹H NMR: (300 MHz, Chloroform-d) δ (ppm) 1.34 (d, J=6.9 Hz, 3H), 1.81-1.98 (m, 2H), 2.23 (s, 3H), 2.86-2.98 (m, 2H), 3.07 (dt, J=9.4, 4.4 Hz, 3H), 3.15 (q, J=8.1, 7.4 Hz, 2H), 3.50 (d, J=4.0 Hz, 2H), 3.61 (dq, J=18.4, 10.6, 8.9 Hz, 3H), 3.75 (q, J=7.6 Hz, 2H), 3.90 (q, J=6.5, 4.7 Hz, 4H), 5.66 (dt, J=8.9, 2.0 Hz, 1H), 5.92 (q, J=6.9 Hz, 1H), 6.30-6.46 (m, 2H), 6.47 (d, J=5.6 Hz, 1H), 6.59 (s, 1H), 6.63 (s, 1H), 7.41 (s, 1H), 7.99 (d, J=5.6 Hz, 1H).

MP: 151.3° C. (Mettler Toledo MP50), uncorrected.

OR: −30.17° (589 nm, c 0.12 v/v, MeOH, 23.8° C.).

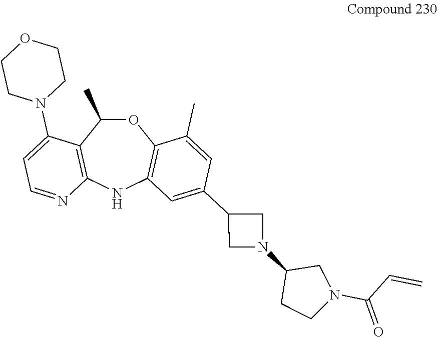 [SMILES: C=CC(=O)N1CC[C@@H](N2CC(c3cc(C)c4c(c3)Nc3nccc(N5CCOCC5)c3[C@@H](C)O4)C2)C1]

(7*R, 20*R), pure isomer but absolute stereochemistry undetermined

Compound 230 was synthesized in a similar manner as Compound 29 using Intermediate 521 instead of Intermediate 229.

LC MS: confirms the MW (RT: 1.707, [M+H]⁺: 490.2. Method: 2).

¹H NMR: (300 MHz, Chloroform-d) δ (ppm) 1.33 (d, J=6.9 Hz, 3H), 1.86-1.98 (m, 2H), 2.23 (s, 3H), 2.91 (dt, J=11.8, 4.5 Hz, 2H), 2.99-3.10 (m, 3H), 3.10-3.20 (m, 2H), 3.44-3.53 (m, 2H), 3.61 (dq, J=13.5, 6.7, 5.7 Hz, 3H), 3.74 (q, J=7.8 Hz, 2H), 3.89 (t, J=4.6 Hz, 4H), 5.67 (dd, J=9.6, 2.7 Hz, 1H), 5.93 (q, J=6.9 Hz, 1H), 6.31-6.54 (m, 2H), 6.47 (d, J=5.6 Hz, 1H), 6.55 (s, 1H), 6.63 (s, 1H), 7.25 (s, 1H), 8.00 (d, J=5.6 Hz, 1H).

MP: 163.1° C. (Mettler Toledo MP50), uncorrected.

OR: +31.71° (589 nm, c 0.13 w/v, MeOH, 22.9° C.).

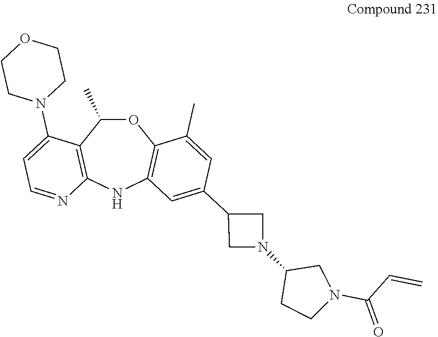 [SMILES: C=CC(=O)N1CC[C@H](N2CC(c3cc(C)c4c(c3)Nc3nccc(N5CCOCC5)c3[C@H](C)O4)C2)C1]

(7*S, 20*S), pure isomer but absolute stereochemistry undetermined Compound 231 was synthesized in a similar manner as Compound 29 using Intermediate 523 instead of Intermediate 229.

LC MS: confirms the MW (RT: 2.774, [M+H]⁺: 490.3, Method: 14).

¹H NMR: (300 MHz, Chloroform-d) δ (ppm) 1.33 (d, J=6.9 Hz, 3H), 1.76-2.05 (m, 2H), 2.23 (s, 3H), 2.84-2.99 (m, 2H), 3.00-3.12 (m, 3H), 3.12-3.21 (m, 2H), 3.50 (d, J=3.9 Hz, 2H), 3.53-3.70 (m, 3H), 3.76 (q, J=8.9 Hz, 2H), 3.83-3.96 (m, 4H), 5.62-5.71 (m, 1H), 5.93 (q, J=6.9 Hz, 1H), 6.32-6.54 (m, 2H), 6.47 (d, J=5.6 Hz, 1H), 6.56 (s, 1H), 6.63 (s, 1H), 7.31 (s, 1H), 7.99 (d, J=5.6 Hz, 1H).

MP: 163° C. (Mettler Toledo MP50), uncorrected.

OR: −52.97° (589 nm, c 0.12 w/v, MeOH, 23.5° C.).

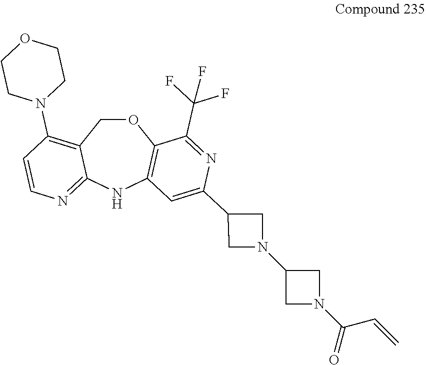 [SMILES: C=CC(=O)N1CC(N2CC(c3cc4c(c(C(F)(F)F)n3)OCc3c(N5CCOCC5)ccnc3N4)C2)C1]

A mixture of Intermediate 534 (200 mg, 0.43 mmol) and Triethylamine (0.12 mL, 0.86 mmol) in DCM (10 mL) was cooled in an ice-water bath. Acryloyl chloride (CAS: 814-68-6) (48 μL, 0.58 mmol) was added and the reaction was stirred for 30 min. The reaction was concentrated to dryness and the residue was dissolved in DCM and washed with a mixture of 1 M Na₂CO₃ and Brine. The organic layer was dried over MgSO₄, filtered, and concentrated to dryness. The product was purified by reverse phase chromatography (Phenomenex Gemini C18 100×30 mm 5 μm; gradient from [95% H₂O−5% ACN-MeOH] to [63% H₂O−37% ACN-MeOH], [0.1% HCOOH]), neutralized with Na₂CO₃ solid and extracted with DCM. The organic layer was dried over MgSO₄, filtered, and concentrated to dryness. Trituration of the residue with Diethylether afforded Compound 235 as a solid (90 mg, yield: 40%).

LC MS: confirms the MW (RT: 1.904, [M+H]⁺: 517, Method: 2)

¹H NMR (300 MHz, CDCl₃) δ (ppm) 8.08 (d, J=5.4 Hz, 1H), 7.75 (s, 1H), 6.90 (s, 1H), 6.54 (d, J=5.5 Hz, 1H), 6.33 (d, J=16.9 Hz, 1H), 6.21 (dd, J=16.9, 10.1 Hz, 1H), 5.66 (d, J=10.0 Hz, 1H), 5.11 (s, 2H), 4.34-4.06 (m, 3H), 3.97-3.83 (m, 5H), 3.79-3.67 (m, 3H), 3.67-3.53 (m, 2H), 3.53-3.41 (m, 1H), 3.07-2.94 (m, 4H)

MP: 136.6° C. (Mettler Toledo MP50), uncorrected.

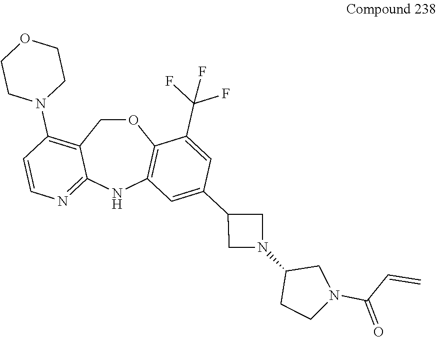 [SMILES: C=CC(=O)N1CC[C@H](N2CC(c3cc4c(c(C(F)(F)F)c3)OCc3c(N5CCOCC5)ccnc3N4)C2)C1]

(*S), pure isomer but absolute stereochemistry undetermined

A mixture of Intermediate 544 (220 mg, 0.46 mmol) and Triethylamine (0.13 mL, 0.93 mmol) in DCM (10 mL) was cooled in an ice-water bath. Acryloyl chloride (CAS: 814-68-6) (51 μL, 0.63 mmol) was added and the reaction was stirred for 30 min. The reaction was concentrated to dryness and the residue was dissolved in DCM and washed with a mixture of 1 M Na₂CO₃ and Brine. The organic layer was dried over MgSO₄, filtered, and concentrated to dryness. The product was purified by reverse phase chromatography (Phenomenex Gemini C18 100×30 mm 5 μm; gradient from [95% H₂O−5% ACN-MeOH] to [63% H₂O−37% ACN-MeOH], [0.1% HCOOH]), neutralized with Na₂CO₃ solid and extracted with DCM. The organic layer was dried over MgSO₄, filtered, and concentrated to dryness. Trituration of the residue with DIPE afforded Compound 238 as a solid (90 mg, yield: 36%)

LC MS: confirms the MW (RT: 1.701, [M+H]⁺: 530, Method: 2)

¹H NMR (300 MHz, CDCl₃) δ (ppm) 7.99 (d, J=5.4 Hz, 1H), 7.64 (s, 1H), 7.01 (s, 2H), 6.55-6.30 (m, 3H), 5.67 (d, J=8.7 Hz, 1H), 5.09 (s, 2H), 3.92-3.83 (m, 4H), 3.77-3.51 (m, 6H), 3.51-3.45 (m, 1H), 3.21-3.04 (m, 3H), 3.03-2.94 (m, 4H), 2.01-1.72 (m, 2H)

MP: 214.9° C. (Mettler Toledo MP50), uncorrected.

OR: −1.190 (589 nm, c 0.084 w/v, MeOH, 23.0° C.).

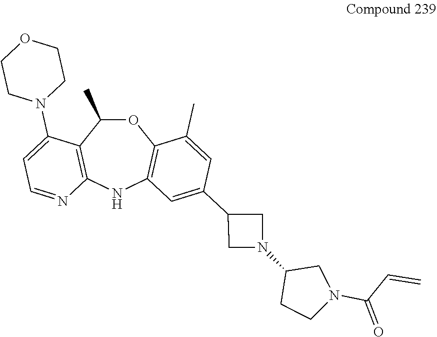 [SMILES: C=CC(=O)N1CC[C@H](N2CC(c3cc(C)c4c(c3)Nc3nccc(N5CCOCC5)c3[C@@H](C)O4)C2)C1]

(7*R, 20*S), pure isomer but absolute stereochemistry undetermined

Compound 239 was synthesized in a similar manner as Compound 29 using Intermediate 522 instead of Intermediate 229.

LC MS: confirms the MW (RT: 1.520, [M+H]⁺: 490.3, Method: 2)

¹H NMR: (300 MHz, Chloroform-d) δ (ppm) 1.34 (d, J=6.9 Hz, 3H), 1.91 (td, J=14.2, 13.4, 6.9 Hz, 2H), 2.23 (s, 3H), 2.91 (dt, J=12.0, 4.6 Hz, 2H), 3.06 (dt, J=9.3, 4.5 Hz, 3H), 3.11-3.21 (m, 2H), 3.31-3.51 (m, 2H), 3.61 (dq, J=12.4, 6.1, 4.5 Hz, 3H), 3.73 (t, J=7.3 Hz, 2H), 3.89 (t, J=4.6 Hz, 4H), 5.67 (dd, J=10.1, 2.5 Hz, 1H), 5.93 (q, J=6.9 Hz, 1H), 6.31-6.53 (m, 2H), 6.47 (d, J=5.5 Hz, 1H), 6.56 (s, 1H), 6.63 (d, J=5.0 Hz, 1H), 8.00 (d, J=5.5 Hz, 1H).

MP: 139.6° C. (Mettler Toledo MP50), uncorrected.

OR: +34.58° (589 nm, c 0.13 w/v, MeOH, 23.0° C.).

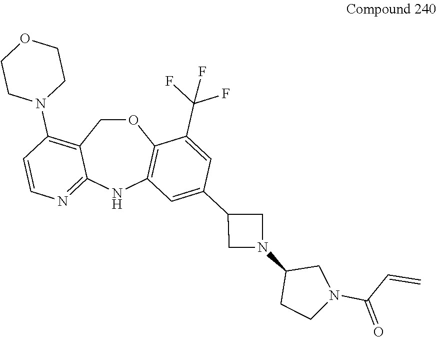 [SMILES: C=CC(=O)N1CC[C@@H](N2CC(c3cc4c(c(C(F)(F)F)c3)OCc3c(N5CCOCC5)ccnc3N4)C2)C1]

(*R), pure isomer but absolute stereochemistry undetermined

A mixture of Intermediate 545 (200 mg, 0.42 mmol) and Triethylamine (0.12 mL, 0.84 mmol) in DCM (10 mL) was cooled in an ice-water bath. Acryloyl chloride (CAS: 814-68-6) (46 μL, 0.57 mmol) was added and the reaction was stirred for 30 min. The reaction was concentrated to dryness and the residue was dissolved in DCM and washed with a mixture of 1 M Na₂CO₃ and Brine. The organic layer was dried over MgSO₄, filtered, and concentrated to dryness. The product was purified by reverse phase chromatography (Phenomenex Gemini C18 100×30 mm 5 μm; gradient: from [95% H₂O−5% ACN-MeOH] to [63% H₂O−37% ACN-MeOH], [0.1% HCOOH]), neutralized with Na₂CO₃ solid and extracted with DCM. The organic layer was dried over MgSO₄, filtered, and concentrated to dryness. Trituration of the residue with DIPE afforded Compound 240 as a solid (77 mg, yield: 34%)

LC MS: confirms the MW (RT: 1.690, [M+H]⁺: 530, Method: 2)

¹H NMR (300 MHz, CDCl₃) δ (ppm) 8.01 (d, J=5.6 Hz, 1H), 7.47 (s, 1H), 7.00 (s, 2H), 6.53-6.31 (m, 3H), 5.75-5.61 (m, 1H), 5.10 (s, 2H), 3.99-3.80 (m, 4H), 3.79-3.51 (m, 6H), 3.51-3.45 (m, 1H), 3.20-3.05 (m, 3H), 3.05-2.91 (m, 4H), 2.01-1.71 (m, 2H)

MP: 104.5° C. (Mettler Toledo MP50), uncorrected.

OR: +1.79° (589 nm, c 0.078 w/v, MeOH, 23.0° C.).

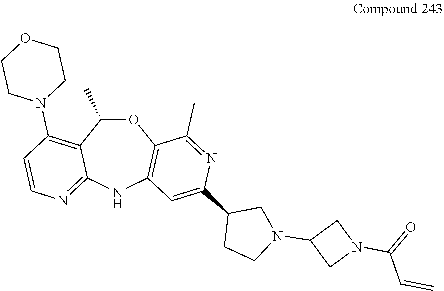 [SMILES: C=CC(=O)N1CC(N2CC[C@@H](c3cc4c(c(C)n3)O[C@@H](C)c3c(N5CCOCC5)ccnc3N4)C2)C1]

(*S, *R), pure isomer, absolute stereochemistry undetermined

Compound 243 was synthesized following a similar sequence of reactions as for the synthesis of Compound 245 starting from Intermediate 555B instead of Intermediate 555A.

LC MS: confirms the MW (RT: 1.562, [M+H]⁺: 491, Method: 2)

¹H NMR (300 MHz, Chloroform-d) d (ppm) 1.36 (d, J=7.0 Hz, 3H), 2.05 (s, 3H), 2.53 (s, 6H), 2.84-2.97 (m, 4H), 2.99-3.16 (m, 2H), 3.91 (d, J=4.8 Hz, 4H), 4.07 (s, 1H), 4.10-4.26 (m, 2H), 4.28 (s, 1H), 5.62-5.73 (m, 1H), 5.90-6.04 (m, 1H), 6.11-6.27 (m, 1H), 6.28-6.40 (m, 1H), 6.61 (s, 2H), 7.26 (s, 1H), 8.09 (d, J=5.8 Hz, 1H).

MP: 144.6° C. (Mettler Toledo MP50), uncorrected.

OR: +19.93° (589 nm, c 0.14 w/v, CHCl₃, 23° C.).

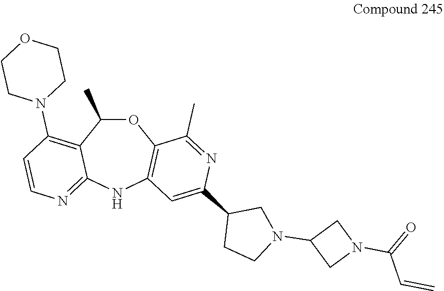 [SMILES: C=CC(=O)N1CC(N2CC[C@@H](c3cc4c(c(C)n3)O[C@H](C)c3c(N5CCOCC5)ccnc3N4)C2)C1]

(*R, *R), pure stereoisomer but absolute stereochemistry undetermined

Triethylamine (185 μL, 1.33 mmol) was added to a solution of Intermediate 556 (116 mg, 0.27 mmol) in DCM (15 mL). The mixture was cooled in an ice bath, then a solution of acryloyl chloride (CAS: 814-68-6) (22 μL, 0.27 mmol) in DCM (4 mL) was added dropwise and the reaction mixture was stirred at room temperature for 2 h. The reaction mixture was quenched by addition of saturated aqueous NaHCO₃ and extracted with DCM. The organic layer was separated, dried over MgSO₄, filtered, and concentrated to dryness. A purification was performed via reverse phase chromatography (Phenomenex Gemini C18 100A column 100 mm×30 mm I.D.; 5 μm particles; gradient from 70% HCOOH aqueous solution (0.1%+ACN 10%)/30% (ACN/MeOH 1/1) to 27% NH₄OAc aqueous solution (0.1%+ACN 10%)/73% (ACN/MeOH 1/1)) to afford Compound 245 as a solid (55 mg, yield: 43%).

LC MS: confirms the MW (RT: 1.565, [M+H]⁺: 491, Method: 2)

NMR: ¹H NMR (300 MHz, Chloroform-d) d (ppm) 1.36 (d, J=6.9 Hz, 3H), 2.05 (dt, J=13.4, 6.9 Hz, 1H), 2.35 (s, 1H), 2.46 (s, 3H), 2.77 (q, J=8.1 Hz, 3H), 2.84-3.13 (m, 5H), 3.48 (d, J=10.5 Hz, 3H), 3.81-3.97 (m, 4H), 4.04 (s, 1H), 4.15 (dd, J=10.8, 6.8 Hz, 2H), 4.21-4.31 (m, 1H), 5.66 (dt, J=10.0, 2.2 Hz, 1H), 5.96 (q, J=6.9 Hz, 1H), 6.12-6.26 (m, 1H), 6.27-6.39 (m, 1H), 6.57 (d, J=5.8 Hz, 2H), 8.07 (d, J=5.5 Hz, 1H).

MP: 138° C. (Mettler Toledo MP50), uncorrected.

OR: +74.4° (589 nm, c 0.14 w/v, CHCl₃, 23.0° C.).

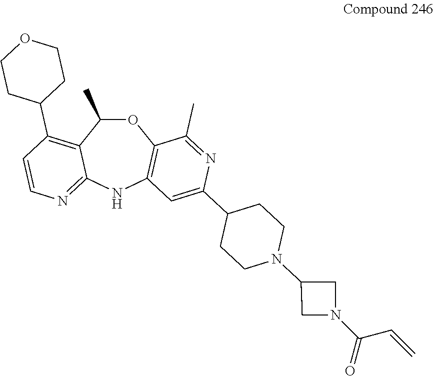 [SMILES: C=CC(=O)N1CC(N2CCC(c3cc4c(c(C)n3)O[C@H](C)c3c(C5CCOCC5)ccnc3N4)CC2)C1]

(*R), pure isomer but absolute stereochemistry undetermined

A mixture of Intermediate 561 (200 mg, 0.44 mmol) and Triethylamine (0.12 mL, 0.89 mmol) in DCM (10 mL) was cooled in an ice-water bath. Acryloyl chloride (CAS: 814-68-6) (43 μL, 0.53 mmol) in DCM (4 mL) was added and the reaction was stirred for 5 min. The reaction was quenched with H₂O and a 1 M Na₂CO₃ solution. The product was extracted with DCM and the combined organic layer was dried over MgSO₄, filtered, and concentrated to dryness. The product was purified by reverse phase chromatography (Phenomenex Gemini C 18 100×30 mm 5 μm; gradient from [72% H₂O−28% ACN-MeOH] to [36% H₂O−64% ACN-MeOH], [H₂O: 25 mM NH₄HCO₃]). Trituration of the residue with diethyl ether afforded Compound 246 as a solid (140 mg, yield: 62%).

LC MS: confirms the MW (RT: 1.383, [M+H]⁺: 504, Method: 2)

¹H NMR (300 MHz, DMSO-d₆) δ (ppm) 9.60 (s, 1H), 8.09 (d, J=5.1 Hz, 1H), 6.89 (s, 1H), 6.83 (d, J=5.1 Hz, 1H), 6.31 (dd, J=17.0, 10.2 Hz, 1H), 6.09 (d, J=17.0 Hz, 1H), 5.99-5.91 (m, 1H), 5.66 (d, J=10.3 Hz, 1H), 4.24 (t, J=7.8 Hz, 1H), 4.09-3.99 (m, 1H), 3.93 (d, J=8.3 Hz, 2H), 3.74 (dd, J=10.0, 4.9 Hz, 1H), 3.64-3.47 (m, 2H), 3.28-3.08 (m, 2H), 2.88 (t, J=11.5 Hz, 2H), 2.42 (t, J=11.7 Hz, 2H), 2.31 (s, 3H), 1.98-1.47 (m, 10H), 1.20 (d, J=6.7 Hz, 3H)

MP: 211.9° C. (Mettler Toledo MP50), uncorrected.

OR: +25.29° (589 nm, c 0.1633 w/v, MeOH, 23.0° C.).

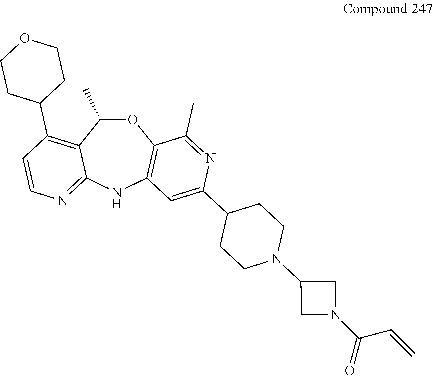 [SMILES: C=CC(=O)N1CC(N2CCC(c3cc4c(c(C)n3)O[C@@H](C)c3c(C5CCOCC5)ccnc3N4)CC2)C1]

(*S), pure isomer but absolute stereochemistry undetermined

Compound 247 was synthesized following a similar sequence of reactions as for the synthesis of Compound 246 starting from Intermediate 560B instead of Intermediate 560A.

LC MS: confirms the MW (RT: 1.349, [M+H]⁺: 504, Method: 2)

¹H NMR (300 MHz, CDCl₃) δ (ppm) 8.13 (d, J=4.9 Hz, 1H), 6.79 (d, J=4.9 Hz, 1H), 6.48 (s, 1H), 6.35 (d, J=16.4 Hz, 1H), 6.20 (dd, J=16.9, 10.2 Hz, 1H), 5.91 (q, J=7.0 Hz, 1H), 5.66 (d, J=10.1 Hz, 1H), 4.24 (t, J=7.6 Hz, 1H), 4.19-4.06 (m, 4H), 4.03-3.92 (m, 1H), 3.58 (t, J=11.0 Hz, 2H), 3.30-3.17 (m, 1H), 3.17-2.88 (m, 3H), 2.17-1.96 (m, 4H), 1.95-1.71 (m, 5H), 1.69-1.50 (m, J=14.5 Hz, 6H), 1.36 (d, J=6.9 Hz, 3H)

MP: 211.7° C. (Mettler Toledo FP62), uncorrected.

OR: −15.35° (589 nm, c 0.0667 w/v, MeOH. 23.0° C.).

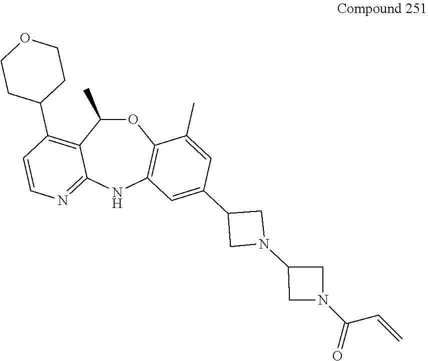 [SMILES: C=CC(=O)N1CC(N2CC(c3cc(C)c4c(c3)Nc3nccc(C5CCOCC5)c3[C@@H](C)O4)C2)C1]

(*R), pure isomer but absolute stereochemistry undetermined

Compound 251 (31 mg, yield: 31%) was synthesized in a similar manner as Compound 245 using Intermediate 569 (87 mg, 0.21 mmol) instead of Intermediate 561.

LC MS: confirms the MW (RT: 1.584, [M+H]⁺: 475, Method: 2)

¹H NMR (300 MHz, CDCl₃) δ (ppm) 8.02 (d, J=5.4 Hz, 1H), 7.47 (br s, 1H), 6.71-6.57 (m, 3H), 6.33 (d, J=17.0 Hz, 1H), 6.20 (dd, J=16.9, 10.1 Hz, 1H), 5.87 (q, J=6.9 Hz, 1H), 5.66 (d, J=10.1 Hz, 1H), 4.23 (t, J=7.9 Hz, 1H), 4.17-3.99 (m, 4H), 3.93 (dd, J=10.5, 4.3 Hz, 1H), 3.76-3.54 (m, 6H), 3.29-3.19 (m, 2H), 3.06 (t, J=11.4 Hz, 1H), 2.25 (s, 3H), 1.94-1.77 (m, 3H), 1.59 (d, J=13.0 Hz, 1H), 1.31 (d, J=6.9 Hz, 3H)

MP: 192.3° C. (Mettler Toledo MP50), uncorrected.

OR: +12.24° (589 nm, c 0.082 w/v, MeOH, 23.0° C.).

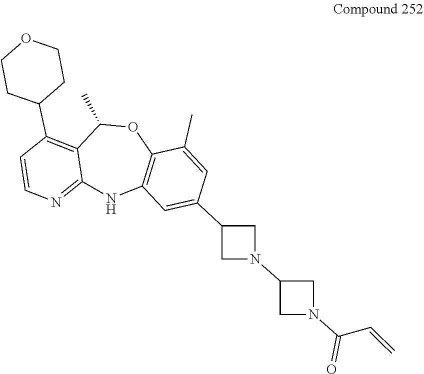 [SMILES: C=CC(=O)N1CC(N2CC(c3cc(C)c4c(c3)Nc3nccc(C5CCOCC5)c3[C@H](C)O4)C2)C1]

(*S), pure isomer but absolute stereochemistry undetermined

Compound 252 was synthesized following a similar sequence of reactions as for the synthesis of Compound 251 using Intermediate 568B instead of Intermediate 568A.

LC MS: confirms the MW (RT: 1.651, [M+H]⁺: 475, Method: 2)

¹H NMR (300 MHz, CDCl₃) δ (ppm) 8.10 (s, 1H), 7.99 (d, J=5.4 Hz, 1H), 6.73-6.63 (m, 3H), 6.32 (d, J=16.9 Hz, 1H), 6.19 (dd, J=17.0, 10.1 Hz, 1H), 5.87 (q, J=6.9 Hz, 1H), 5.67 (d, J=10.1 Hz, 1H), 4.23 (t, J=7.9 Hz, 1H), 4.17-4.00 (m, 4H), 3.92 (dd, J=10.5, 4.1 Hz, 1H), 3.80-3.43 (m, 6H), 3.30-3.19 (m, 2H), 3.07 (t, J=11.5 Hz, 1H), 2.25 (s, 3H), 1.98-1.75 (m, 3H), 1.59 (d, J=13.0 Hz, 1H), 1.31 (d, J=6.9 Hz, 3H)

MP: 183.7° C. (Mettler Toledo FP62), uncorrected.

OR: −12.5° (589 nm, c 0.076 w/v, MeOH, 23.3° C.).

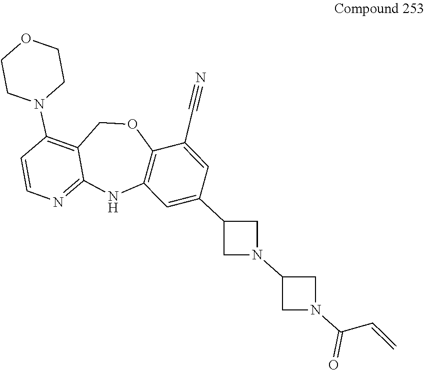 [SMILES: C=CC(=O)N1CC(N2CC(c3cc(C#N)c4c(c3)Nc3nccc(N5CCOCC5)c3CO4)C2)C1]

Acryloyl chloride (CAS: 814-68-6) (0.061 mL, 0.75 mmol) was added to a solution of Intermediate 578 (284 mg, 0.68 mmol) in Et₃N (0.29 mL, 2.09 mmol) and DCM (25 mL) at 0° C. and the reaction mixture was stirred at room temperature for 2 h. The reaction mixture was poured onto a mixture of water and DCM and this mixture was stirred for 10 min at room temperature then the organic layer was dried over MgSO₄, filtered, and concentrated to dryness. Purification by column chromatography (Irregular SiOH 15-40 μm 40 g GraceResolv®, mobile phase gradient from 98% DCM, 2% MeOH, 0.2% NH₄OH to 84% DCM, 14% MeOH, 1.4% NH₄OH) and the product was triturated with ACN, filtered, and dried to afford Compound 253 as a solid (65 mg, yield: 24%).

LC MS: confirms the MW (RT: 2.24, [M+H]⁺: 473, Method: 1)

¹H NMR (DMSO-d₆, 500 MHz) δ (ppm) 9.42 (s, 1H), 8.05 (d, 1H, J=5.4 Hz), 7.55 (d, 1H, J=1.9 Hz), 7.15 (d, 1H, J=1.9 Hz), 6.55 (d, 1H, J=5.7 Hz), 6.32 (dd, 1H, J=10.2, 16.9 Hz), 6.09 (dd, 1H, J=1.9, 17.0 Hz), 5.66 (dd, 1H, J=2.2, 10.4 Hz), 5.13 (s, 2H), 4.21 (t, 1H, J=8.0 Hz), 3.9-4.0 (m, 2H), 3.7-3.8 (m, 5H), 3.5-3.6 (m, 4H), 3.1-3.2 (m, 2H), 2.9-3.0 (m, 4H), 2.08 (s, 1H)

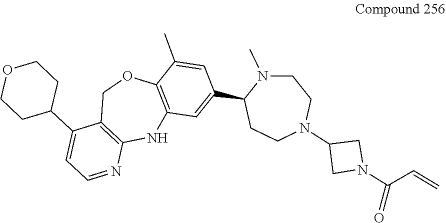 [SMILES: C=CC(=O)N1CC(N2CC[C@@H](c3cc(C)c4c(c3)Nc3nccc(C5CCOCC5)c3CO4)N(C)CC2)C1]

(*S) pure stereoisomer but absolute stereochemistry undetermined

A solution of acryloyl chloride (CAS: 814-68-6) (0.031 mL, 0.38 mmol) in DCM (2 mL) was added to a solution of Intermediate 588 (135 mg, 0.29 mmol) and Et₃N (0.082 mL, 0.58 mmol) in DCM (18 mL) at 0° C. and the reaction mixture was stirred at 0° C. for 30 min. The reaction mixture was quenched with water and a 1 M Na₂CO₃ solution and extracted with DCM. The combined organic layer was dried over MgSO₄, filtered, and concentrated to dryness. Chromatography over silica gel (25 g column, gradient of MeOH in DCM from 0 to 10%) followed by a further purification by column chromatography (Phenomenex Gemini C 18 100×30 mm 5 μm; gradient of ACN/MeOH, 1/1, v/v in 0.1% aqueous formic acid from 5 to 37%) and trituration with diethyl ether afforded Compound 256 (45 mg, yield: 30%).

LC MS: confirms the MW (RT: 1.880, [M+H]⁺: 518, Method: 3)

¹H NMR (300 MHz, CDCl₃) δ (ppm) 8.05 (d, J=5.2 Hz, 1H), 6.92 (s, 1H), 6.74 (s, 1H), 6.65 (d, J=5.1 Hz, 2H), 6.32 (d, J=17.0 Hz, 1H), 6.18 (dd, J=16.9, 10.1 Hz, 1H), 5.66 (d, J=10.1 Hz, 1H), 5.10 (s, 2H), 4.21 (dd, J=17.1, 8.7 Hz, 1H), 4.15-3.99 (m, 4H), 3.97-3.84 (m, 1H), 3.58 (t, J=11.3 Hz, 2H), 3.15-2.90 (m, 3H), 2.84-2.36 (m, 5H), 2.25 (s, 6H), 1.88-1.76 (m, 2H), 1.73-1.51 (m, 5H)

MP: 108.3° C. (Mettler Toledo MP50), uncorrected.

OR: +4.58° (589 nm, c 0.135 w/v, MeOH, 23.0° C.).

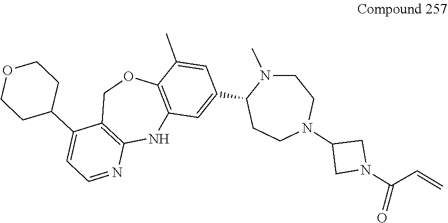 [SMILES: C=CC(=O)N1CC(N2CC[C@H](c3cc(C)c4c(c3)Nc3nccc(C5CCOCC5)c3CO4)N(C)CC2)C1]

(*R), pure isomer but absolute stereochemistry undetermined

Compound 257 was synthesized following a similar sequence of reactions as for the synthesis of Compound 256 using Intermediate 587B instead of Intermediate 587A.

LC MS: confirms the MW (RT: 1.880, [M+H]⁺: 518, Method: 3)

¹H NMR (300 MHz, CDCl₃) δ (ppm) 8.05 (d, J=5.2 Hz, 1H), 6.94 (s, 1H), 6.73 (s, 1H), 6.68-6.58 (m, 2H), 6.32 (d, J=16.6 Hz, 1H), 6.18 (dd, J=16.9, 10.2 Hz, 1H), 5.65 (d, J=10.0 Hz, 1H), 5.10 (s, 2H), 4.26-4.14 (m, 1H), 4.14-3.98 (m, 4H), 3.97-3.84 (m, 1H), 3.58 (t, J=11.5 Hz, 2H), 3.42-3.29 (m, 2H), 3.16-2.90 (m, 3H), 2.83-2.69 (m, 1H), 2.68-2.51 (m, 2H), 2.51-2.36 (m, 1H), 2.24 (s, 6H), 2.09-1.95 (m, 2H), 1.93-1.75 (m, 2H), 1.67 (d, J=12.5 Hz, 2H)

MP: 131.5° C. (Mettler Toledo FP62), uncorrected.

OR: −23.2432° (589 nm, c 0.148 w/v. MeOH, 23.0° C.).

Compound 258 and compound 268

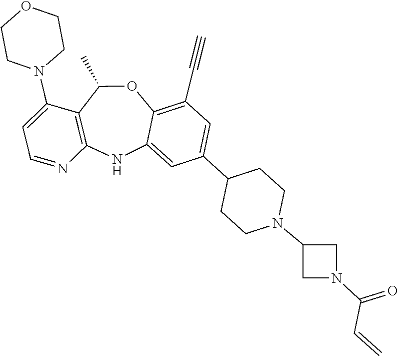 [SMILES: C#Cc1cc(C2CCN(C3CN(C(=O)C=C)C3)CC2)cc2c1O[C@@H](C)c1c(N3CCOCC3)ccnc1N2]

Compound 258 (*S), pure isomer but absolute stereochemistry undetermined

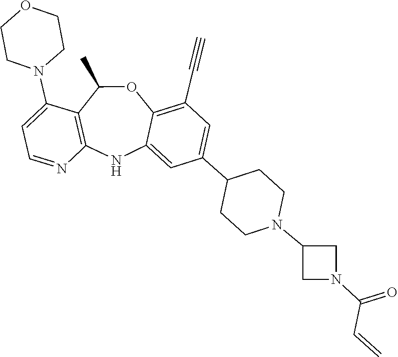 [SMILES: C#Cc1cc(C2CCN(C3CN(C(=O)C=C)C3)CC2)cc2c1O[C@H](C)c1c(N3CCOCC3)ccnc1N2]

Compound 268 (*R), pure isomer but absolute stereochemistry undetermined Et₃N (0.55 mL, 0.4 mmol) was added to a stirred solution of Intermediate 597 (67 mg, 0.13 mmol) in DCM (5.2 mL). The mixture was cooled down to 0° C. and acryloyl chloride (CAS: 814-68-6) (12 μL, 0.14 mmol) was added dropwise. The mixture was stirred at 0° C. for 4 h, then the mixture was diluted with water and extracted with DCM. The organic layer was separated, washed with water, brine, dried over Na₂SO₄, filtered, and concentrated in vacuo. The filtrate was concentrated in vacuo, was triturated with a mixture of heptane/EtOAc 70/30 and filtered, to give a yellow solid which was purified by reverse phase HPLC (Stationary phase: C18 XBridge 30×100 mm 5 μm), Mobile phase: Gradient from 75% NH₄HCO₃ 0.25% solution in Water, 25% ACN to 57% NH₄HCO₃ 0.25% solution in Water, 43% ACN), followed by chiral SFC (Stationary phase: CHIRALPAK AD-H 5 μm 250*30 mm, Mobile phase: 40% CO₂, 60% iPrOH (0.6% Et₃N)) to give Compound 258 (*S) (24 mg, yield: 36%) and Compound 268 (*R) (11 mg, yield: 16%).

### Data for Compound 258:

LC MS: confirms the MW (RT: 2.55, [M+H]⁺: 514, Method: 1)

¹H NMR (400 MHz, DMSO-d₆, 22° C.): δ (ppm) 9.30 (s, 1H), 8.05 (d, J=5.6 Hz, 1H), 7.14 (d, J=2.0 Hz, 1H), 6.77 (d, J=2.0 Hz, 1H), 6.62 (d, J=5.6 Hz, 1H), 6.31 (dd, J=17.1, 10.3 Hz, 1H), 6.10 (dd, J=17.0, 2.3 Hz, 1H), 5.88 (q, J=6.9 Hz, 1H), 5.64-5.69 (m, 1H), 4.24 (t, J=7.9 Hz, 1H), 4.16 (s, 1H), 4.04 (br dd, J=9.2, 5.3 Hz, 1H), 3.95 (br dd, J=10.0, 7.6 Hz, 1H), 3.70-3.83 (m, 4H), 3.09-3.23 (m, 1H), 2.78-2.96 (m, 5H), 2.34-2.46 (m, 1H), 1.82-1.94 (m, 2H), 1.76 (br d, J=11.5 Hz, 2H), 1.39-1.65 (m, 2H), 1.20-1.30 ppm (m, 4H)

SFC: RT: 1.43, 100%, [M+H]⁺ 514, Method: 6

### Data for Compound 268:

LC MS: confirms the MW (RT: 2.55, [M+H]⁺: 514, Method: 1)

¹H NMR (500 MHz, DMSO-d₆, 21° C.): δ (ppm) 9.29 (s, 1H), 8.05 (d, J=5.4 Hz, 1H), 7.14 (d, J=2.2 Hz, 1H), 6.77 (d, J=2.2 Hz, 1H), 6.62 (d, J=5.4 Hz, 1H), 6.31 (dd, J=16.9, 10.2 Hz, 1H), 6.12 (d, J=2.2 Hz, 1H), 5.86-5.91 (m, 1H), 5.65-5.68 (m, 1H), 4.24 (t, J=8.0 Hz, 1H), 4.16 (s, 1H), 4.04 (dd, J=9.0, 5.2 Hz, 1H), 3.94 (dd, J=10.1, 7.6 Hz, 1H), 3.70-3.82 (m, 5H), 3.12-3.17 (m, 1H), 2.82-2.94 (m, 6H), 1.86-1.93 (m, 2H), 1.76 (br d, J=10.4 Hz, 2H), 1.52-1.61 (m, 2H), 1.25 ppm (d, J=6.9 Hz, 3H)

SFC: RT: 1.02, 100%, [M+H]⁺ 514, Method: 6

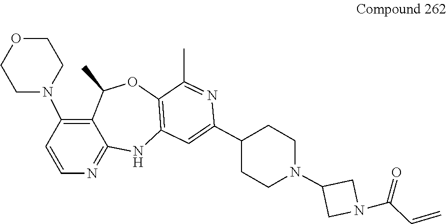 [SMILES: C=CC(=O)N1CC(N2CCC(c3cc4c(c(C)n3)O[C@H](C)c3c(N5CCOCC5)ccnc3N4)CC2)C1]

(*R), pure enantiomer but absolute stereochemistry undetermined

Et₃N (544 μL, 3.905 mmol, 5 eq.) was added to a solution of Intermediate 76 (352 mg, 0.781 mmol) in DCM (8 mL). The reaction mixture was cooled to 0° C. and acryloyl chloride (63 μL, 0.781 mmol, 1 eq.) in DCM (2 mL) was added dropwise. The reaction mixture was stirred at room temperature for 3 h. The reaction was quenched by addition of saturated aqueous NaHCO₃ and the mixture was extracted with DCM. The organic layer was concentrated under vacuum and the residue was purified by was purified by reverse phase column chromatography (Phenomenex Gemini C18 30×100 mm 5 μm; gradient from 95% [0.1% HCOOH]−5% [ACN:MeOH (1:1)] to 63% [0.1% HCOOH]−37% [ACN:MeOH (1:1)]) to afford Compound 262 (193 mg, yield: 49%) as a white solid.

LCMS: confirms the MW (RT: 1.34 [M+H]⁺ 505, Method: 2).

MP: 225° C. (Mettler Toledo MP50), uncorrected.

OR: +32.0° (589 nm, c 0.12 w/v, DMSO, 23° C.).

¹H NMR (300 MHz, Chloroform-d) d (ppm) 1.36 (d, J=6.9 Hz, 3H), 1.69-1.89 (m, 2H), 1.93-2.10 (m, 4H), 2.47 (s, 3H), 2.85-3.03 (m, 4H), 3.02-3.13 (m, 3H), 3.15-3.28 (n. 1H), 3.90 (dt, J=5.4, 2.7 Hz, 4H), 3.97 (dd, J=10.5, 5.3 Hz. 1H), 4.05-4.18 (m, 2H), 4.24 (t, J=7.9 Hz, 1H), 5.66 (dd, J=10.0, 2.2 Hz, 1H), 5.96 (q, J=6.9 Hz, 1H), 6.19 (dd, J=17.0, 10.1 Hz, 1H), 6.34 (d, J=17.0 Hz, 1H), 6.43 (s, 1H), 6.58 (d, J=5.5 Hz, 1H), 7.26 (s, 1H), 8.07 (d, J=5.6 Hz, 1H).

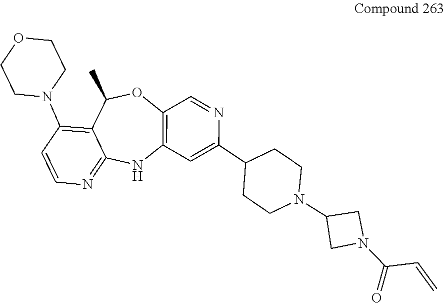 [SMILES: C=CC(=O)N1CC(N2CCC(c3cc4c(cn3)O[C@H](C)c3c(N5CCOCC5)ccnc3N4)CC2)C1]

(*R), pure stereoisomer but absolute stereochemistry undetermined

Et₃N (102 μL, 0.733 mmol, 5 eq.) was added to a solution of Intermediate 57 (64 mg, 0.147 mmol) in DCM (4 mL). The reaction mixture was cooled in an ice bath and acryloyl chloride (12 μL, 0.147 mmol, 1 eq.) in DCM (1 mL) was added dropwise. The reaction mixture was stirred at room temperature for 3 h. The reaction was quenched by addition of saturated aqueous NaHCO₃ and the mixture was extracted with DCM. The organic layer was concentrated under vacuum and the residue was purified by reverse phase column chromatography (Phenomenex Gemini C18 30×100 mm 5 μm; gradient from 72% [25 mM NH₄HCO₃]−28% [ACN:MeOH (1:1)] to 36% [25 mM NH₄HCO₃]−64% [ACN:MeOH (1:1)]) to give Compound 263 (25 mg, 35%) as a white solid.

LCMS: confirms the MW (RT: 1.42 [M+H]⁺ 491, Method: 2).

MP: >300° C. (Mettler Toledo MP50), uncorrected.

OR: +36° (589 nm, c 0.10 w/v, MeOH, 23° C.).

¹H NMR (300 MHz, Chloroform-d) d (ppm) 1.41 (d, J=6.9 Hz, 3H), 1.67-1.92 (m, 2H), 1.94-2.10 (m, 4H), 2.65 (td, J=12.1, 6.1 Hz, 1H), 2.84-3.03 (m, 4H), 3.04-3.16 (m, 2H), 3.21 (p, J=6.4 Hz, 1H), 3.80-3.92 (m, 41H), 3.92-4.02 (m, 1H), 4.06-4.18 (m, 2H), 4.23 (t, J=7.9 Hz, 1H), 5.66 (dd, J=10.1, 2.1 Hz, 1H), 5.87 (q, J=6.9 Hz, 1H), 6.19 (dd, J=17.0, 10.1 Hz, 1H), 6.33 (dd, J=17.1, 2.1 Hz, 1H), 6.53 (s, 1H), 6.55 (d, J=5.7 Hz, 1H), 7.32 (s, 1H), 8.06 (d. J=5.6 Hz. 1H), 8.09 (s, 1H).

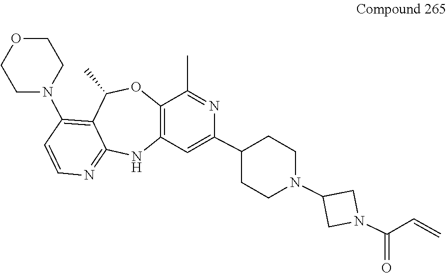 [SMILES: C=CC(=O)N1CC(N2CCC(c3cc4c(c(C)n3)O[C@@H](C)c3c(N5CCOCC5)ccnc3N4)CC2)C1]

(*S), pure enantiomer but absolute stereochemistry undetermined

Compound 265 was prepared using a procedure analogous to Compound 262, starting from Intermediate 77 instead of Intermediate 76.

LCMS: confirms the MW (RT: 1.28 [M+H]⁺ 505, Method: 2).

MP: 288.5° C. (Mettler Toledo MP50), uncorrected.

OR: −34.6° (589 nm, c 0.1533 w/v, 23° C.).

¹H NMR (300 MHz, Chloroform-d) d (ppm)1.36 (d, J=6.9 Hz, 3H), 1.65-1.83 (m, 2H), 1.94-2.10 (m, 4H), 2.47 (s, 3H), 2.86-3.02 (m, 4H), 3.02-3.15 (m, 3H), 3.16-3.26 (m, 1H), 3.86-3.93 (m, 4H), 3.97 (dd, J=10.5, 5.3 Hz, 1H), 4.07-4.17 (m, 2H), 4.23 (t, J=7.9 Hz, 1H), 5.66 (dd, J=10.1, 2.1 Hz, 1H), 5.96 (q, J=6.9 Hz, 1H), 6.19 (dd, J=17.0, 10.1 Hz, 1H), 6.34 (dd, J=17.1, 2.1 Hz, 1H), 6.45 (s, 1H), 6.58 (d, J=5.5 Hz, 1H), 7.26 (s, 1H), 8.07 (d, J=5.5 Hz, 1H).

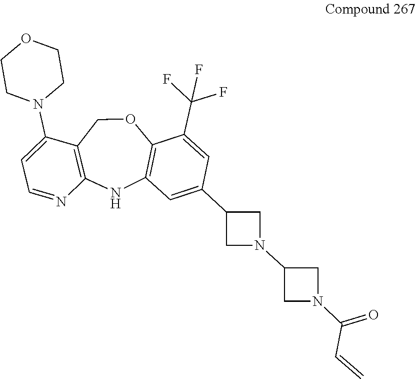 [SMILES: C=CC(=O)N1CC(N2CC(c3cc4c(c(C(F)(F)F)c3)OCc3c(N5CCOCC5)ccnc3N4)C2)C1]

NaOtBu (2 M in THF, 0.035 mL, 0.07 mmol) was added to a solution of Intermediate 605 (28 mg, 0.047 mmol) in THF (1.1 mL) at 0° C. The reaction mixture was stirred at 0° C. for 30 min then at room temperature for 20 min. Water and DCM were added and this mixture was stirred for 10 min at room temperature. The organic layer was decanted with Chromabond®, the solvent was evaporated until dryness and the residue was purified by column chromatography (Irregular SiOH 15-40 μm 40 g GraceResolv®, Gradient from: 98% DCM, 2% MeOH, 0.2% NH₄OH to 94% DCM, 6% MeOH, 0.6% NH₄OH) to afford Compound 267 (34 mg, yield: 140%).

LC MS: confirms the MW (RT: 2.56, [M+H]⁺: 515, Method: 1)

¹H NMR (500 MHz, DMSO-d₆, 25° C.): δ (ppm) 9.39 (s, 1H), 8.04 (d, J=5.4 Hz, 1H), 7.54 (d, J=1.9 Hz, 1H), 7.00 (d, J=1.9 Hz, 1H), 6.53 (d, J=5.7 Hz, 1H), 6.31 (dd, J=16.9, 10.2 Hz, 1H), 6.09 (dd, J=17.0, 2.2 Hz, 1H), 5.64-5.68 (m, 1H), 5.06 (s, 2H), 4.22 (t, J=7.9 Hz, 1H), 3.84-4.04 (m, 2H), 3.73-3.80 (m, 4H), 3.71 (dd, J=10.4, 4.4 Hz, 1H), 3.56-3.64 (m, 3H), 3.46-3.53 (m, 1H), 3.13 (q, J=5.5 Hz, 2H), 2.84-2.90 ppm (m, 4H)

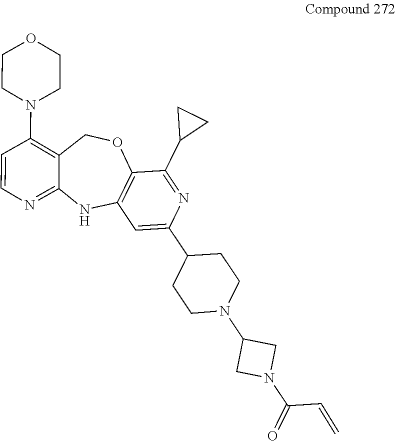 [SMILES: C=CC(=O)N1CC(N2CCC(c3cc4c(c(C5CC5)n3)OCc3c(N5CCOCC5)ccnc3N4)CC2)C1]

Intermediate 89 (158 mg, 0.342 mmol) was dissolved in a mixture of Et₃N (238 μL. 1.708 mmol, 5 eq.) and DCM (12 mL). The mixture was cooled to 0° C. and acryloyl chloride (38 μL, 0.444 mmol, 1.3 eq.) was added. The reaction mixture was stirred for 30 min. The reaction reaction was concentrated to dryness. The residue was dissolved in DCM and washed with Na₂CO₃ (1 M in water, 10 mL). The organic layer was dried over MgSO₄, filtered, and evaporated. The residue was purified by reverse phase chromatography (Phenomenex Gemini C18 100×30 mm 5 μm; gradient from 95% H₂O−5% ACN/MeOH to 63% H₂O−37% ACN/MeOH, 0.1% HCOOH). The desired fractions were concentrated and the obtained solid was triturated in Et₂O to give Compound 272 (22 mg, yield: 12%) as a white solid.

LCMS: confirms the MW (RT: 1.45 [M+H]⁺ 517, Method: 2).

MP: 189.9° C. (METTLER Toledo FP62), 10° C./min, uncorrected.

¹H NMR (300 MHz, CDCl₃) δ (ppm) 8.00 (d, J=5.8 Hz, 1H), 7.47 (s, 1H), 6.47 (d, J=5.7 Hz, 1H), 6.37 (d, J=8.7 Hz, 1H), 6.30 (s, 1H), 6.19 (dd, J=17.0, 10.2 Hz, 1H), 5.66 (d, J=10.1 Hz, 1H), 5.08 (s, 2H), 4.24 (t, J=7.7 Hz, 1H), 4.19-4.07 (m, 2H), 4.05-3.96 (m, 1H), 3.92-3.85 (m, J=4.1 Hz, 4H), 3.27-3.16 (m, 1H), 3.09-3.02 (m, J=4.1 Hz, 4H), 3.00-2.89 (m, 2H), 2.64-2.47 (m, J=4.3 Hz, 2H), 1.95-1.75 (m, 6H), 1.13-1.04 (m, 2H), 0.95-0.87 (m, 2H)

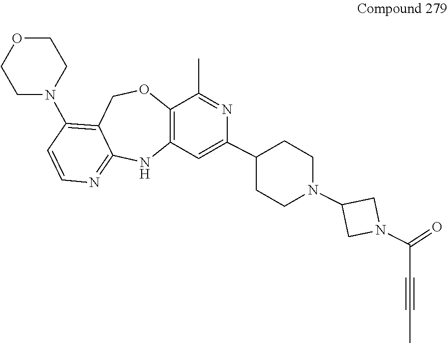 [SMILES: CC#CC(=O)N1CC(N2CCC(c3cc4c(c(C)n3)OCc3c(N5CCOCC5)ccnc3N4)CC2)C1]

HBTU (430 mg, 1.134 mmol, 1.5 eq.) was added to a solution of 2-butynoic acid (95 mg, 1.134 mmol, 1.5 eq.) and DIPEA (257 μL, 1.512 mmol, 2 eq.) in DCM (20 mL) at room temperature. The mixture was stirred for 15 min before addition of Intermediate 65 (330 mg, 0.756 mmol). The resulting mixture was stirred at room temperature for 2 h. The reaction mixture was diluted with DCM (50 mL) and washed with Na₂CO₃ (1 M in water, 25 mL). The aqueous layer was extracted again with DCM (25 mL). The combined organic layers were dried over MgSO₄, filtered, and concentrated. The residue was purified by flash column chromatography (silica gel, DCM:MeOH (9:1)/DCM, from 0/100 to 70/30). The obtained solid was recrystallized in ACN, filtered, and dried to give Compound 279 (194 mg, yield: 49%) as a white solid.

LCMS: confirms the MW (RT: 1.34 [M+H]⁺ 503, Method: 2).

MP: 178.5° C. (Mettler Toledo FP62), 10° C./min, uncorrected.

¹H NMR (300 MHz, DMSO-d₆) δ (ppm) 12.19 (s, 1H), 9.57 (s, 1H), 8.06 (d, J=5.1 Hz, 1H), 7.67 (s, 1H), 7.61 (s, 1H), 6.92 (s, 1H), 6.58 (d, J=5.3 Hz, 1H), 5.14 (s, 2H), 4.16 (t, J=7.9 Hz, 1H), 4.00-3.89 (m, 2H), 3.86-3.76 (m, 4H), 3.75-3.65 (m, 1H), 3.22-3.08 (m, 1H), 3.01-2.84 (m, 6H), 2.00 (s, 3H), 1.97-1.72 (m, J=24.0 Hz, 6H)

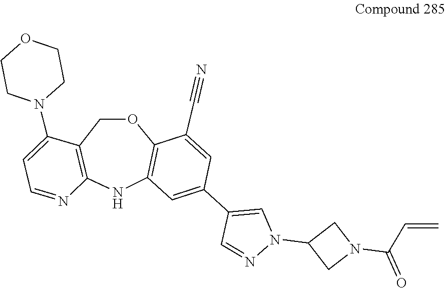 [SMILES: C=CC(=O)N1CC(n2cc(-c3cc(C#N)c4c(c3)Nc3nccc(N5CCOCC5)c3CO4)cn2)C1]

Intermediate 611 (120 mg, 0.28 mmol) was taken in DCM (9 mL), then Et₃N (78 μL, 0.56 mmol) was added and the mixture was cooled in iced-water. Acryloyl chloride (CAS: 814-68-6) (30 μL, 0.36 mmol) was added dropwise in DCM (1 mL) was added and reaction stirred for 0.5 h at 0° C. The reaction mixture was quenched with 1 M Na₂CO₃, the organics were extracted with DCM, dried over MgSO₄, filtered, and concentrated to dryness. Chromatography over silica gel (25 g column, gradient of MeOH in DCM from 0 to 10%) followed by reverse phase chromatography (Phenomenex Gemini C18 100×30 mm 5 μm; gradient of ACN in 0.1% aqueous formic acid from 5 to 37%) was followed by basification to pH 8 with 1 M Na₂CO₃, and extraction with DCM. The organic solution was dried over MgSO₄, filtered, and concentrated. Further trituration in diethyl ether gave Compound 285 (52 mg, yield: 37%).

LC MS: confirms the MW (RT: 2.207, [M+H]⁺: 484, Method: 3)

¹H NMR (300 MHz, DMSO-d₆) δ (ppm) 9.37 (s, 1H), 8.30 (s, 1H), 8.06 (d, J=5.4 Hz, 1H), 7.91 (s, 1H), 7.74 (s, 1H), 7.43 (s, 1H), 6.56 (d, J=5.5 Hz, 1H), 6.37 (dd, J=16.9, 10.3 Hz, 1H), 6.16 (d, J=17.1 Hz, 1H), 5.72 (d, J=10.2 Hz, 1H), 5.39-5.28 (m, 1H), 5.16 (s, 2H), 4.71 (t, J=8.5 Hz, 1H), 4.57-4.35 (m, 2H), 4.24-4.19 (m, 1H), 3.83-3.74 (m, 4H), 2.99-2.86 (m, 4H)

MP: 225.6° C. (Mettler Toledo FP62) uncorrected.

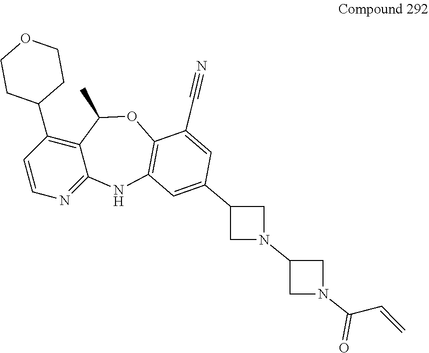 [SMILES: C=CC(=O)N1CC(N2CC(c3cc(C#N)c4c(c3)Nc3nccc(C5CCOCC5)c3[C@@H](C)O4)C2)C1]

(*R), pure isomer but absolute stereochemistry undetermined

Intermediate 620 (47 mg, 0.11 mmol) was dissolved in DCM (6 mL), then Et₃N (30 μL, 0.22 mmol) was added and the mixture was cooled in iced-water. Acryloyl chloride (CAS: 814-68-6) (1.31 mL, 0.1 M in DCM, 0.13 mmol) was added dropwise and the reaction mixture was stirred for 0.5 h at 0° C. The reaction mixture was concentrated to dryness, then diluted with DCM and washed with 1 M Na₂CO₃. The organics were extracted with DCM, dried over MgSO₄, filtered, and concentrated to dryness. Chromatography over silica gel (silica gel, DCM+MeOH (9/1)/DCM, from 0/100 to 60/40). to afford Compound 292 (30 mg, yield: 56%).

LC MS: confirms the MW (RT: 1.873, [M+H]⁺: 486, Method: 2)

¹H NMR (300 MHz, CDCl₃) δ (ppm) 8.08 (d, J=4.9 Hz, 1H), 7.67 (s, 1H), 7.10-7.00 (m, 2H), 6.75 (d, J=5.0 Hz, 1H), 6.34 (d, J=16.7 Hz, 1H), 6.19 (dd, J=16.8, 10.2 Hz, 1H), 5.99 (q, J=6.8 Hz, 1H), 5.67 (d, J=10.0 Hz, 1H), 4.23 (t, J=7.5 Hz, 1H), 4.16-4.05 (m, 3H), 4.04-3.97 (m, 1H), 3.90 (d, J=6.8 Hz, 1H), 3.70 (t, J=6.2 Hz, 2H), 3.66-3.42 (m, 4H), 3.29-3.18 (m, 2H), 3.14-2.98 (m, 1H), 1.95-1.74 (m, 3H), 1.65-1.48 (m, 1H), 1.44 (d, J=6.7 Hz, 3H)

MP: 154.2° C. (Mettler Toledo FP62), uncorrected.

OR: +0.0595° (589 nm, c 0.1073 w/v, MeOH, 23.0° C.).

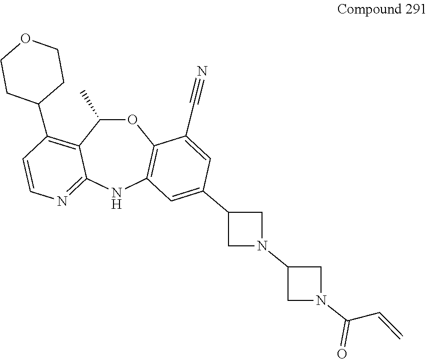 [SMILES: C=CC(=O)N1CC(N2CC(c3cc(C#N)c4c(c3)Nc3nccc(C5CCOCC5)c3[C@H](C)O4)C2)C1]

(*S), pure isomer but absolute stereochemistry undetermined

Compound 291 was synthesized following a similar sequence of reactions as for the synthesis of Compound 292 using Intermediate 619B instead of Intermediate 619A.

LC MS: confirms the MW (RT: 1.878, [M+H]⁺: 486, Method: 2)

¹H NMR (300 MHz, CDCl₃) δ (ppm) 8.09 (d, J=4.9 Hz, 1H), 7.54 (s, 1H), 7.12-6.97 (m, 2H), 6.74 (d, J=4.9 Hz, 1H), 6.33 (d, J=16.7 Hz, 1H), 6.19 (dd, J=16.8, 10.2 Hz, 1H), 5.98 (q, J=6.9 Hz, 1H), 5.67 (d, J=10.0 Hz, 1H), 4.22 (t, J=7.6 Hz, 1H), 4.18-4.06 (m, 3H), 4.06-3.96 (m, 1H), 3.90 (d, J=7.0 Hz, 1H), 3.77-3.66 (m, 2H), 3.66-3.43 (m, 4H), 3.29-3.17 (m, 2H), 3.04 (t, J=10.8 Hz, 1H), 1.98-1.73 (m, 3H), 1.59 (d, J=12.7 Hz, 1H), 1.44 (d, J=6.7 Hz, 3H)

MP: 136.8° C. (Mettler Toledo FP62), uncorrected.

OR: −0.0531° (589 nm, c 0.110667 w/v, MeOH, 23.0° C.).

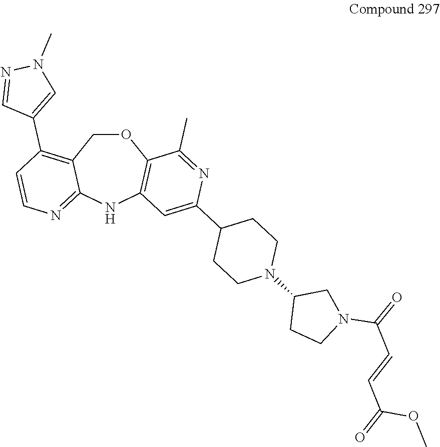 [SMILES: COC(=O)/C=C/C(=O)N1CC[C@H](N2CCC(c3cc4c(c(C)n3)OCc3c(-c5cnn(C)c5)ccnc3N4)CC2)C1]

(*S. E), pure isomer but absolute stereochemistry undetermined

HATU [CAS: 148893-10-1] (0.38 g, 1.01 mmol) was added to a solution of Mono-Methyl fumarate [CAS: 2756-87-8] (130 mg, 1.01 mmol) and DIPEA (0.23 mL, 1.35 mmol) in DCM (20 mL) at room temperature. The mixture was stirred for 15 min before addition of Intermediate 502 (300 mg, 0.67 mmol), then the reaction was stirred for 0.5 h. The reaction mixture was diluted with DCM and washed with 1 M Na₂CO₃. The aqueous layer was extracted with DCM (25 mL). The combined organic layers were dried over MgSO₄, filtered, concentrated to dryness, and purified by chromatography over silica gel (gradient of MeOH in DCM from 0 to 100%) to afford Compound 297 (249 mg, yield: 65%).

LC MS: confirms the MW (RT: 1.383, [M+H]⁺: 558, Method: 2)

¹H NMR (300 MHz, CDCl₃) δ (ppm) 8.11 (d, J=5.0 Hz, 1H), 7.59 (s, 1H), 7.52 (s, 1H), 7.38 (s, 1H), 7.23 (d, J=15.7 Hz, 1H), 6.89 (dd, J=15.2, 11.4 Hz, 1H), 6.79 (d, J=5.0 Hz, 1H), 6.53 (d, J=15.3 Hz, 1H), 5.14 (s, 2H), 4.01 (s, 3H), 4.08-3.84 (m, 2H), 3.80 (s, 3H), 3.59 (dd, J=17.2, 10.1 Hz, 1H), 3.41-3.26 (m, 1H), 3.14 (d, J=10.4 Hz, 1H), 3.09-2.79 (m, 2H), 2.77-2.58 (m, 1H), 2.46 (s, 3H), 2.33-2.11 (m, 3H), 2.09-1.88 (m, 3H), 1.88-1.66 (m, 2H)

MP: 208.2° C. (Mettler Toledo FP62), uncorrected.

OR: −18.505° (589 nm, c 0.1227 w/v, MeOH, 23.0° C.).

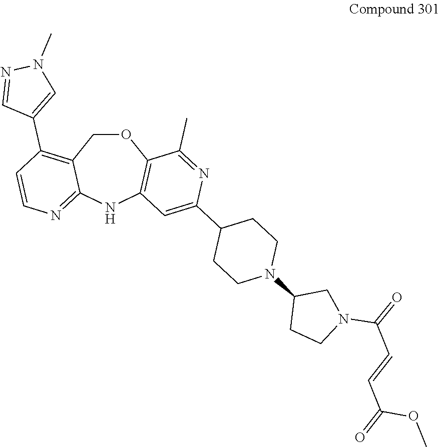 [SMILES: COC(=O)/C=C/C(=O)N1CC[C@@H](N2CCC(c3cc4c(c(C)n3)OCc3c(-c5cnn(C)c5)ccnc3N4)CC2)C1]

(*R, E), pure isomer but absolute stereochemistry undetermined

Compound 301 was synthesized following the procedure used for Compound 297 using Intermediate 501 instead of Intermediate 502, purified by column chromatography over silica gel (gradient of DCM/MeOH (9:1) in DCM from 0% to 100%) and triturated with diethyl ether.

LC MS: confirms the MW (RT: 1.390, [M+H]⁺: 558, Method: 2)

¹H NMR (300 MHz, CDCl₃) δ (ppm) 8.11 (d, J=5.0 Hz, 1H), 7.59 (s, 1H), 7.52 (s, 1H), 7.38 (s, 1H), 7.23 (d, J=15.7 Hz, 1H), 6.89 (dd, J=15.2, 11.4 Hz, 1H), 6.79 (d, J=5.0 Hz, 1H), 6.53 (d, J=15.3 Hz, 1H), 5.14 (s, 2H), 4.01 (s, 3H), 4.08-3.84 (m, 2H), 3.80 (s, 3H), 3.59 (dd, J=17.2, 10.1 Hz, 1H), 3.41-3.26 (m, 1H), 3.14 (d, J=10.4 Hz, 1H), 3.09-2.79 (m, 2H), 2.77-2.58 (m, 1H), 2.46 (s, 3H), 2.33-2.11 (m, 3H), 2.09-1.88 (m, 3H), 1.88-1.66 (m, 2H)

MP: 209.9° C. (Mettler Toledo FP62), uncorrected.

OR: +18.475° (589 nm, c 0.118 w/v. MeOH, 23.0° C.).

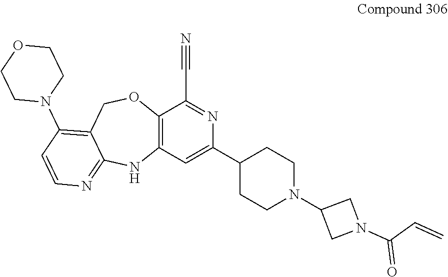 [SMILES: C=CC(=O)N1CC(N2CCC(c3cc4c(c(C#N)n3)OCc3c(N5CCOCC5)ccnc3N4)CC2)C1]

NaOtBu (25 mg, 0.264 mmol, 1.3 eq.) was added to a solution of Intermediate 97 (118 mg, 0.203 mmol) in THF (5 mL), at −15° C., and the mixture was stirred at room temperature for 3 h. More NaOtBu (6 mg, 0.3 eq.) was added and the reaction mixture was stirred at room temperature for 3 h. The reaction mixture was poured onto ice/water and extracted with DCM. The solvent was evaporated and the residue was purified by reverse phase chromatography (Phenomenex Gemini C18 30×100 mm 5 μm; gradient from 72% [25 mM NH₄HCO₃]−28% [ACN:MeOH (1:1)] to 36%) [25 mM NH₄HCO₃]−64% [ACN:MeOH (1:1)]) to afford Compound 306 (39 mg, yield: 38%) as a white solid.

LCMS: confirms the MW (RT: 1.63 [M+H]⁺ 502, Method: 2).

MP: 256.7° C. (Mettler Toledo MP50), uncorrected.

¹H NMR (300 MHz, Chloroform-d) d (ppm) 1.69-1.86 (m, 2H), 1.91-2.11 (m, 4H), 2.58-2.73 (m, 1H), 2.88-3.04 (m, 2H), 3.08 (t, J=4.5 Hz, 4H), 3.23 (p, J=6.4 Hz, 1H), 3.90 (t, J=4.6 Hz, 4H), 3.94-4.04 (m, 1H), 4.07-4.19 (m, 2H), 4.24 (t, J=7.9 Hz, 1H), 5.16 (s, 2H), 5.67 (dd, J=10.1, 2.1 Hz, 1H), 6.19 (dd, J=16.9, 10.2 Hz, 1H), 6.35 (dd, J=17.0, 2.2 Hz, 1H), 6.54 (d, J=5.6 Hz, 1H), 6.77 (s, 1H), 7.53 (s, 1H), 8.08 (d, J=5.6 Hz, 1H).

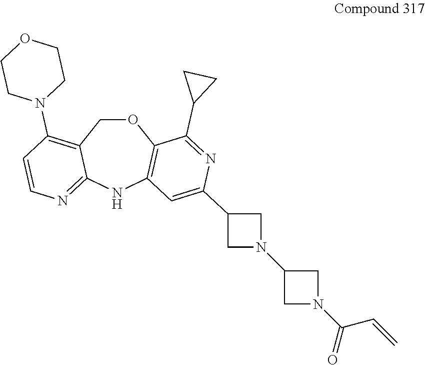 [SMILES: C=CC(=O)N1CC(N2CC(c3cc4c(c(C5CC5)n3)OCc3c(N5CCOCC5)ccnc3N4)C2)C1]

Intermediate 645 (298 mg, 0.69 mmol) was dissolved in DCM (12 mL), then Et₃N (478 μL, 3.43 mmol) was added and the mixture was cooled in ice and water. A solution of acryloyl chloride (CAS: 814-68-6) (56 μL, 0.69 mmol) in DCM was added dropwise and the reaction mixture was stirred for 3 h at room temperature. The reaction mixture was quenched by the addition of a saturated aqueous solution of NaHCO₃ and extracted with DCM. The combined organic layers were dried over MgSO₄, filtered, concentrated to dryness and purified by reverse phase chromatography (Phenomenex Gemini C18 100A column (100 mm×30 mm I.D.; 5 μm particles); gradient from 70% of a NH₄OAc aqueous solution (65 mM+ACN 10%)/30% (ACN/MeOH 1/1) to 27% of a NH₄OAc aqueous solution (65 mM+ACN 10%)/73% (ACN/MeOH 1/1)) to afford Compound 317 (99 mg, yield: 30%).

LC MS: confirms the MW (RT: 1.666, [M+H]⁺: 489, Method:2)

¹H NMR (300 MHz, Chloroform-d) d (ppm) 0.81-0.91 (m, 1H), 0.92-1.00 (m, 2H), 1.03-1.11 (m, 2H), 2.49-2.63 (m, 1H), 3.03 (t, J=4.5 Hz, 4H), 3.43-3.61 (m, 2H), 3.61-3.68 (m, 1H), 3.69-3.78 (m, 2H), 3.89 (t, J=4.6 Hz, 4H), 3.94-4.04 (m, 1H), 4.08-4.21 (m, 1H), 4.21-4.32 (m, 2H), 5.09 (s, 2H), 5.67 (dd, J=10.0, 2.3 Hz, 1H), 6.22 (dd, J=17.0, 9.9 Hz, 1H), 6.31 (d, J=2.3 Hz, 1H), 6.37 (d, J=2.0 Hz, 1H), 6.47 (d, J=5.6 Hz, 1H), 7.16 (s, 1H), 8.02 (d, J=5.6 Hz, 1H).

MP: 131.3° C. (Mettler Toledo MP50), uncorrected.

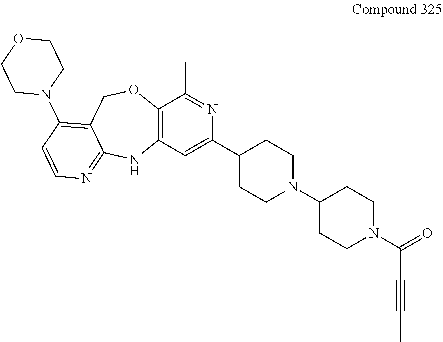 [SMILES: CC#CC(=O)N1CCC(N2CCC(c3cc4c(c(C)n3)OCc3c(N5CCOCC5)ccnc3N4)CC2)CC1]

HBTU [CAS: 94790-37-1] (1.42 g, 3.75 mmol) was added to a solution of Intermediate 652 (1.16 g, 2.5 mmol), 2-butynoic acid [CAS: 590-93-2] (320 mg, 3.75 mmol) and DIPEA (2.18 mL, 12.5 mmol) in DCM (50 mL) at room temperature. The mixture was stirred for 2 h. The reaction mixture was poured into a saturated solution of NaHCO₃ and extracted with EtOAc. The combined organic layers were washed with a saturated solution of NaHCO₃ and brine, dried over MgSO₄, filtered, concentrated to dryness and purified by chromatography over silica gel (gradient of MeOH in DCM) and reverse phase chromatography (Phenomenex Gemini C18 100A column (100 mm×30 mm I.D.; 5 μm particles); gradient from 72% of a NH₄CO₃ aqueous solution (25 mM+ACN 10%)/28% (ACN/MeOH 1/1) to 36% of a NH₄CO₃ aqueous solution (25 mM+ACN 10%)/64% (ACN/MeOH 1/1)) to affordd Compound 325 (636 mg, yield: 48%).

LC MS: confirms the MW (RT: 1.447, [M+H]⁺: 531, Method: 3)

¹H NMR (300 MHz, Chloroform-d) d (ppm) 8.04 (d, J=5.6 Hz, 1H), 6.95 (s, 1H), 6.46 (d, J=5.5 Hz, 1H), 6.43 (s, 1H), 5.05 (s, 2H), 4.61 (d, J=13.4 Hz, 1H), 4.44 (d, J=13.4 Hz, 1H), 3.89 (t, J=4.5 Hz, 4H), 3.20-2.91 (m, 7H), 2.75-2.50 (m, 3H), 2.44 (s, 3H), 2.39-2.23 (m, 1H), 2.01 (s, 3H), 1.99-1.85 (m, 4H), 1.81-1.59 (m, 4H), 1.51 (q, J=11.1, 9.5 Hz, 1H).

MP: 236.7° C. (Mettler Toledo MP50), uncorrected.

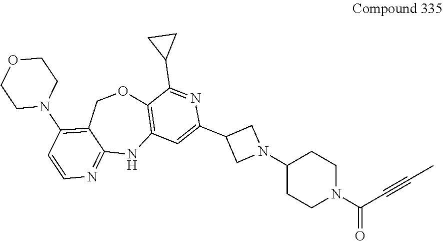 [SMILES: CC#CC(=O)N1CCC(N2CC(c3cc4c(c(C5CC5)n3)OCc3c(N5CCOCC5)ccnc3N4)C2)CC1]

Compound 335 was synthesized following the procedure used for Compound 325using Intermediate 654 instead of Intermediate 652, and 5 eq of DIPEA. Purification by column chromatography over silica gel (gradient of MeOH in DCM) followed by and precipitation with diisopropyl ether afforded Compound 335 (461 mg, yield: 44%).

LC MS: confirms the MW (RT: 1.855, [M+H]⁺: 529, Method: 2)

¹H NMR (300 MHz, Chloroform-d) d (ppm) 0.91-1.01 (m, 2H), 1.05 (dq, J=5.2, 3.3, 2.6 Hz, 2H), 1.34-1.61 (m, 2H), 1.82 (d, J=13.4 Hz, 2H), 2.01 (s, 3H), 2.50-2.63 (m, 1H), 2.64-2.83 (m, 1H), 2.88-2.99 (m, 1H), 3.02 (t, J=4.4 Hz, 4H), 3.26 (t, J=11.9 Hz, 1H), 3.55 (s, 2H), 3.70-3.81 (m, 1H), 3.82-3.98 (m, 6H), 4.34 (t, J=17.9 Hz, 2H), 5.09 (s, 2H), 6.37 (s, 1H), 6.48 (d, J=5.4 Hz, 1H), 7.06 (s, 1H), 8.04 (d, J=5.6 Hz, 1H).

MP: 236.6° C. (Mettler Toledo MP50), uncorrected.

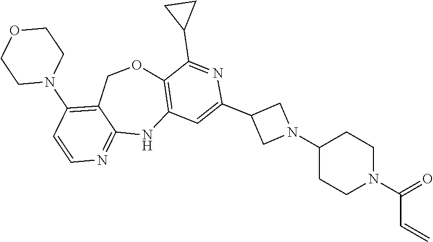 [SMILES: C=CC(=O)N1CCC(N2CC(c3cc4c(c(C5CC5)n3)OCc3c(N5CCOCC5)ccnc3N4)C2)CC1]

DIPEA (1.75 mL, 10 mmol, 5 eq.) was added to a solution of Intermediate 654 (925 mg, 2 mmol) in DCM (30 mL). The reaction mixture was cooled in a ice bath. Then acryloyl chloride (162 μL, 2 mmol, 1 eq.) in DCM (10 mL) was added dropwise. The reaction mixture was stirred at room temperature for 2 h. The reaction was quenched by addition of saturated aqueous NaHCO₃ and the mixture was extracted with DCM. The organic layer was concentrated under vacuum and the residue was purified by flash column chromatography (SiO₂, MeOH-DCM gradient). Part of the desired fractions were evaporated and the residue was precipitated from acetonitrile, filtered, and dried to give a first batch of Compound 336 as a white solid (263 mg, yield: 25%). Another part of the desired fractions were evaporated and the residue was purified by reverse phase column chromatography (Phenomenex Gemini C18 30×100 mm 5 μm; gradient from 95% [0.1% HCOOH]− 5% [ACN:MeOH (1:1)] to 63% [0.1% HCOOH]− 37% [ACN:MeOH (1:1)]) to give a second fraction of Compound 336 as a white solid (145 mg, yield: 14%).

LC-MS: MW confirmed (RT: 1.734, Area %: 99, [M+H]⁺: 517.2, Method: 2)

M.P.: 233.3° C. (Mettler Toledo MP50)

¹H NMR (300 MHz, Chloroform-d) d (ppm) 0.87-0.97 (m, 2H), 0.99-1.14 (m, 2H), 1.31 (ddt, J=15.1, 10.4, 5.3 Hz, 2H), 1.66-1.83 (m, 2H), 2.31-2.46 (m, 1H), 2.53 (dq, J=8.7, 5.2, 4.4 Hz, 1H), 3.02 (1, J=4.5 Hz, 5H), 3.19 (t, J=12.2 Hz, 1H), 3.30 (d, J=6.6 Hz, 2H), 3.63-3.70 (m, 3H), 3.89 (t, J=4.6 Hz, 5H), 4.32 (d, J=13.2 Hz, TH), 5.09 (s, 2H), 5.66 (dd, J=10.4, 2.1 Hz, 1H), 6.24 (dd, J=17.0, 2.2 Hz, 1H), 6.39 (s, 1H), 6.47 (d, J=5.5 Hz, 1H), 6.58 (dd, J=16.8, 10.6 Hz, 1H), 7.01 (s, 1H), 8.04 (d, J 5.5 Hz, 1H).

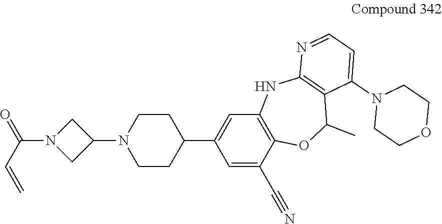 [SMILES: C=CC(=O)N1CC(N2CCC(c3cc(C#N)c4c(c3)Nc3nccc(N5CCOCC5)c3C(C)O4)CC2)C1]

Acryloyl chloride (0.035 mL, 429 mmol, 1.25 eq.) in DCM (2 mL) was added to a solution of Intermediate 657 (158 mg, 0.343 mmol), Et₃N (0.45 mL, 3.237 mmol, 9.4 eq.) in DCM (5 mL) at 0° C. The reaction mixture was stirred for 40 min at room temperature. The mixture was poured into water and was extracted twice with DCM. The organic layer was decanted on ChromabondR and the solvent was evaporated. The residue was purified by column chromatography (Irregular SiO₂ 15-40 μm 40 g GraceResolv®, gradient from 99% DCM, 1% MeOH, 0.1% NH₄OH to 90% DCM, 10% MeOH, 1% NH₄OH). The desired fractions were evaporated and the residue was further purified by reverse phase column chromatography (Stationary phase: YMC-actus Triart C18 10 μm 30*150 mm, Mobile phase: gradient from 65% NH₄HCO₃ 0.2%, 35% ACN to 25% NH₄HCO₃ 0.2%, 75% ACN). The desired fractions were evaporated and the residue was taken up in ACN/water (2 mL/5 mL) and freeze-dried overnight to give Compound 342 (46 mg, yield: 26%).

LC-MS: confirms the MW, RT: 2.37, [M−H]⁺: 515.4, Method: 1)

¹H NMR (DMSO-d₆, 500 MHz) δ (ppm) 9.57 (s, 1H), 8.08 (d, 1H, J=5.7 Hz), 7.57 (d, 1H, J=1.9 Hz), 7.15 (d, 1H, J=1.9 Hz), 6.78 (dd, 1H, J=10.4, 16.7 Hz), 6.67 (d, 1H, J=5.4 Hz), 6.07 (dd, 1H, J=2.5, 16.7 Hz), 5.97 (q, 1H, J=6.9 Hz), 5.64 (dd, 1H, J=2.4, 10.6 Hz), 3.96 (br s, 1H), 3.84 (br d, 1H, J=12.3 Hz), 3.79 (t 4H, J=4.6 Hz), 3.5-3.6 (m, 2H), 3.48 (qum, 1H, J=7.1 Hz), 3.2-3.3 (m, 1H), 3.0-3.1 (m, 3H), 2.9-2.9 (m, 4H), 2.3-2.4 (m, 1H), 1.63 (br s, 2H) 1.33 (d, 3H, J=6.9 Hz), 1.15 (br s, 2H)

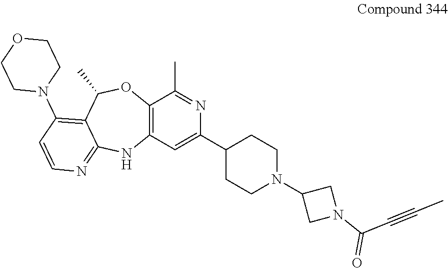 [SMILES: CC#CC(=O)N1CC(N2CCC(c3cc4c(c(C)n3)O[C@@H](C)c3c(N5CCOCC5)ccnc3N4)CC2)C1]

(*S), pure enantiomer but absolute stereochemistry undetermined

DIPEA (2 mL, 11.763 mmol, 5 eq.) was added to a suspension of 2-butynoic acid (237 mg, 2.823 mmol, 1.2 eq.) and HBTU (1.256 g, 3.293 mmol, 1.4 eq.) in DCM (18 mL) at 0° C. under nitrogen atmosphere. The reaction mixture was stirred for 10 min. A solution of Intermediate 77 (1.06 g, 2.353 mmol) in DCM (53 mL) was then added at 0° C. and the reaction mixture was stirred for 45 min. Water, NH₄Cl, and DCM were added. The organic layer was separated and the solvent was evaporated. The residue was purified by column chromatography (Stationary phase: irregular SiOH 15-40 μm 120 g Grace, Mobile phase: gradient from 99% DCM, 1% MeOH, 0.1% NH₄OH to 95% DCM, 5% MeOH, 0.5% NH₄OH). The obtained solid was recrystallized in Et₂O. This solid was then dissolved in DCM and aqueous NH₄Cl. The organic layer was separated and the solvent was evaporated. The residue was diluted with DCM and basified with NH₄OH. The organic layer was separated, washed with aqueous NH₄Cl and the solvent was evaporated. The residue was recrystallized in Et₂O to give Compound 344 (257 mg, yield: 21%).

¹H NMR (DMSO-d₆, 500 MHz) δ (ppm) 9.54 (br s, 1H), 8.08 (d, 1H, J=5.7 Hz), 6.89 (s, 1H), 6.68 (d, 1H, J=5.7 Hz), 5.91 (q, 1H, J=6.9 Hz), 4.14 (t, 1H, J=8.2 Hz), 3.9-4.0 (m, 2H), 3.78 (t, 4H, J=4.6 Hz), 3.71 (dd, 1H, J=5.0, 10.1 Hz), 3.1-3.2 (m, 1H), 2.8-2.9 (m, 6H), 2.4-2.5 (m, 1H), 2.29 (s, 3H), 2.00 (s, 3H), 1.8-1.9 (m, 4H), 1.6-1.7 (m, 2H), 1.24 (d, 3H, J=6.9 Hz)

LCMS: confirms the MW (RT: 2.28, [M+H]⁺ 517, Method: 1).

SFC: RT: 1.11, [M+H]⁺ 517, Method: 1OR: −29.13° (589 nm, c 0.206 w/v %, DMF, 20° C.).

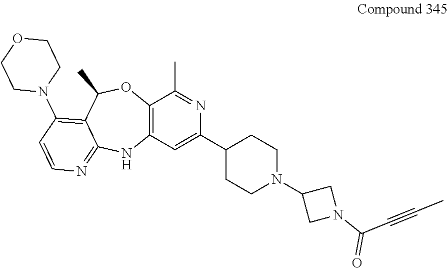 [SMILES: CC#CC(=O)N1CC(N2CCC(c3cc4c(c(C)n3)O[C@H](C)c3c(N5CCOCC5)ccnc3N4)CC2)C1]

(*R), pure enantiomer but absolute stereochemistry undetermined

Compound 345 was prepared using a procedure analogous to Compound 344, starting from Intermediate 76 instead of Intermediate 77.

LCMS: confirms the MW (RT: 2.27, [M+H]⁺ 517, Method: 1).

SFC: RT: 1.50, [M+H]⁺ 517, Method: 1.

OR: +22.32° (589 nm, c 0.224 w/v %, DMF, 20° C.).

¹H NMR (DMSO-d₆, 500 MHz) δ (ppm) 9.52 (s, 1H), 8.08 (d, 1H, J=5.7 Hz), 6.88 (s, 1H), 6.68 (d, 1H, J=5.4 Hz), 5.91 (q, 1H, J=6.9 Hz), 4.14 (t, 1H, J=8.0 Hz), 3.9-4.0 (m, 2H), 3.78 (t, 4H, J=4.4 Hz), 3.70 (dd, 1H, J=5.0, 10.1 Hz), 3.1-3.2 (m, 1H), 2.8-2.9 (m, 6H), 2.4-2.5 (m, 1H), 2.29 (s, 3H), 2.00 (s, 3H), 1.89 (br t, 2H, J=11.7 Hz), 1.80 (br d, 2H, J=11.7 Hz), 1.6-1.7 (m, 2H), 1.24 (d, 3H, J=6.9 Hz)

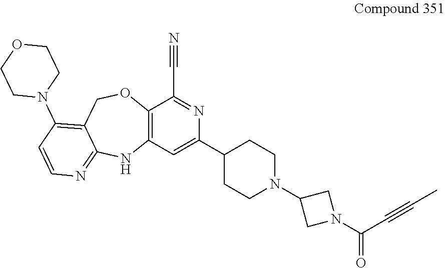 [SMILES: CC#CC(=O)N1CC(N2CCC(c3cc4c(c(C#N)n3)OCc3c(N5CCOCC5)ccnc3N4)CC2)C1]

HBTU (171 ng, 0.452 mmol, 1.5 eq.) was added to a solution of Intermediate 99 (135 mg, 0.301 mmol), 2-butynoic acid (38 mg, 0.452 mmol, 1.5 eq.), and DIPEA (263 μL, 1.505 mmol, 5 eq.) in DCM (10 mL), and the mixture was stirred at room temperature for 2 h. The mixture was poured into saturated aqueous NaHCO₃ and extracted with DCM. The organic layer was washed with aqueous NaHCO₃ and brine, and was evaporated. The residue was purified by reverse phase chromatography (Phenonenex Gemini C18 30×100 mm 5 μm; gradient from 95% [0.1% HCOOH]−5% [ACN:MeOH (1:1)] to 63% [0.1% HCOOH]−37% [ACN:MeOH (1:1)]) to give Compound 351 (24 mg, yield: 15%).

LCMS: confirms the MW (RT: 1.73 [M+H]⁺ 515, Method: 2).

MP: 193.2° C. (Mettler Toledo MP50), uncorrected.

¹H NMR (300 MHz, Chloroform-d) d 8.08 (d, J=5.6 Hz, 1H), 7.23 (s, 1H), 6.72 (s, 1H), 6.54 (d, J=5.6 Hz, 1H), 5.16 (s, 2H), 4.19 (t, J=8.2 Hz, 1H), 4.11-3.99 (m, 2H), 3.96-3.82 (m, 5H), 3.19 (p, J=6.3 Hz, 1H), 3.07 (dd, J=6.1, 3.1 Hz, 4H), 2.93 (d, J=10.7 Hz, 2H), 2.73-2.56 (m, 1H), 2.08-1.89 (m, 7H), 1.77 (t, J=11.6 Hz, 2H).

### Compound 354 and Compound 353

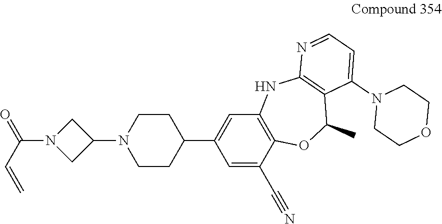 [SMILES: C=CC(=O)N1CC(N2CCC(c3cc(C#N)c4c(c3)Nc3nccc(N5CCOCC5)c3[C@@H](C)O4)CC2)C1] [1822] (*R), pure stereoisomer but absolute stereochemistry undetermined

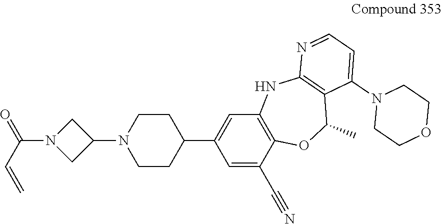 [SMILES: C=CC(=O)N1CC(N2CCC(c3cc(C#N)c4c(c3)Nc3nccc(N5CCOCC5)c3[C@H](C)O4)CC2)C1]

(*S), pure stereoisomer but absolute stereochemistry undetermined

A sample of Compound 342 was purified by chiral SFC (Stationary phase: CHIRALPAK AS-H 5 μm 250*20 mm, Mobile phase: 0.6% Et₃N, 65% CO₂, 35% MeOH). The desired fractions were evaporated and both enantiomers were taken up in ACN/water (2 mL/5 mL) and freeze-dried to give Compound 354 and Compound 353.

### Compound 354

LC-MS: confirms the MW, RT: 2.48, [M+H]⁺ 515.4, Method: 1)

¹H NMR (DMSO-d₆, 500 MHz) δ (ppm) 9.50 (s, 1H), 8.08 (d, 1H, J=5.4 Hz), 7.42 (d, 1H, J=1.9 Hz), 7.07 (d, 1H, J=2.2 Hz), 6.66 (d, 1H, J=5.4 Hz), 6.31 (dd, 1H, J=10.4, 17.0 Hz), 6.10 (dd, 1H, J=2.2, 17.0 Hz), 5.96 (q, 1H, J=6.9 Hz), 5.66 (dd, 1H, J=2.2, 10.1 Hz), 4.24 (t, 1H, J=7.9 Hz), 4.04 (dd, 1H, J=4.9, 9.0 Hz), 3.95 (dd, 1H, J=7.4, 9.9 Hz), 3.79 (t, 4H, J=4.4 Hz), 3.75 (br dd, 1H, J=5.2, 10.2 Hz), 3.1-3.2 (m, 1H), 2.8-2.9 (m, 6H), 2.4-2.5 (m, 1H), 1.9-1.9 (m, 2H), 1.78 (br d, 2H, J=11.0 Hz), 1.59 (q, 2H, J=12.2 Hz), 1.33 (d, 3H, J=6.9 Hz)

OR: +63.64° (589 nm, c 0.22 w/v %, DMF, 20° C.)

### Compound 353

LC-MS: confirms the MW, RT: 2.48, [M+H]⁺ 515.4, Method: 1)

¹H NMR (DMSO-d₆, 500 MHz) δ (ppm) 9.50 (s, 1H), 8.08 (d, 1H, J=5.4 Hz), 7.42 (d, 1H, J=1.9 Hz), 7.07 (d, 1H, J=1.9 Hz), 6.66 (d, 1H, J=5.4 Hz), 6.31 (dd, 1H, J=10.1, 17.0 Hz), 6.10 (dd, 1H, J=2.2, 17.0 Hz), 5.96 (q, 1H, J=6.7 Hz), 5.66 (dd, 1H, J=2.2, 10.4 Hz), 4.24 (t, 1H, J=8.0 Hz), 4.04 (dd, 1H, J=5.2, 9.0 Hz), 3.95 (dd, 1H, J=7.6, 10.1 Hz), 3.79 (t, 4H, J=4.6 Hz), 3.75 (br dd, 1H, J=5.0, 10.4 Hz), 3.1-3.2 (m, 1H), 2.8-2.9 (m, 6H), 2.4-2.5 (m, 1H), 1.9-2.0 (m, 2H), 1.7-1.8 (m, 2H), 1.59 (q, 2H, J=11.9 Hz), 1.33 (d, 3H, J=6.9 Hz)

OR: −65.71° (589 nm, c 0.21 w/v %. DMF, 20° C.)

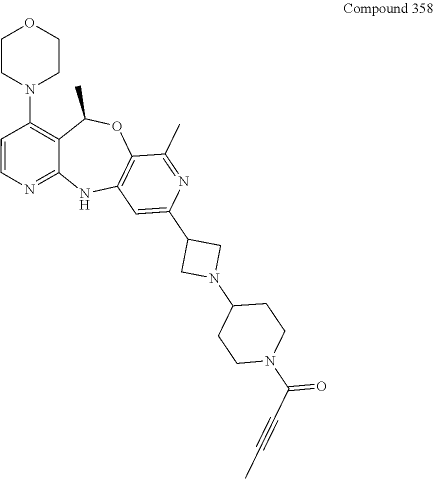 [SMILES: CC#CC(=O)N1CCC(N2CC(c3cc4c(c(C)n3)O[C@H](C)c3c(N5CCOCC5)ccnc3N4)C2)CC1]

(*R), pure stereoisomer but absolute stereochemistry undetermined

HBTU (400 mg, 1.055 mmol, 1.3 eq.) was added to a solution of Intermediate 666 (356 mg, 0.790 mmol), 2-butynoic acid (CAS [590-93-2], 100 mg, 1.185 mmol, 1.5 eq.), and DIPEA (403 μL, 2.37 mmol, 3 eq.) in DCM (10 mL) at room temperature and the reaction mixture was stirred at room temperature for 30 min. Aqueous Na₂CO₃ (1 M, 30 mL) and DCM (50 mL) were added. The organic layer was separated and the aqueous layer was extracted again with DCM (30 mL). The combined organic layers were dried over MgSO₄, filtered, and concentrated. The residue was purified by column chromatography over silica gel (gradient of DCM/MeOH 9/1 in DCM from 0 to 100%) followed by reverse phase chromatography (Phenomenex Gemini C18 100×30 mm 5 μm; gradient from (95% H₂O−5% ACN−MeOH) to (63% H₂O−37% ACN−MeOH]−[0.1% HCOOH]). The desired fractions were neutralized with solid Na₂CO₃ and extracted with DCM. The organic layer was dried over MgSO₄, filtered, and concentrated. The residue was triturated in Et₂O to give Compound 358 (115 mg, yield: 28%) as a solid.

LC-MS: pure (RT: 1.511, Area %: 99, [M−H]⁺: 517, Method: 2)

¹H NMR (300 MHz, DMSO-d₆) δ (ppm) 9.60 (s, 1H), 8.08 (d, J=5.3 Hz, 1H), 7.01 (s, 1H), 6.68 (d, J=5.4 Hz, 1H), 5.91 (q, J=6.9 Hz, 1H), 4.05-3.93 (m, 1H), 3.93-3.83 (m, 1H), 3.82-3.73 (m, 41), 3.59-3.45 (m, 3H), 3.42-3.35 (m, 1H), 3.21-3.13 (m, 2H), 3.12-2.99 (m, 1H), 2.96-2.83 (m, 4H), 2.37-2.26 (m, 1H), 2.30 (s, 3H), 2.01 (s, 3H), 1.76-1.54 (m, 2H), 1.24 (d, J=6.6 Hz, 3H), 1.19-1.00 (m, 2H)

SFC: pure (RT: 8.708, Area %: 99, [M+H]⁺: 517, Method: LuX-Amilose_1-2-propanol 25 to 60% 30° C.)

M.P.: 220.7° C. (Mettler Toledo FP62)

O.R.: +37.5589° (589 nm, c 0.113333 w/v, DMF, 23° C.)

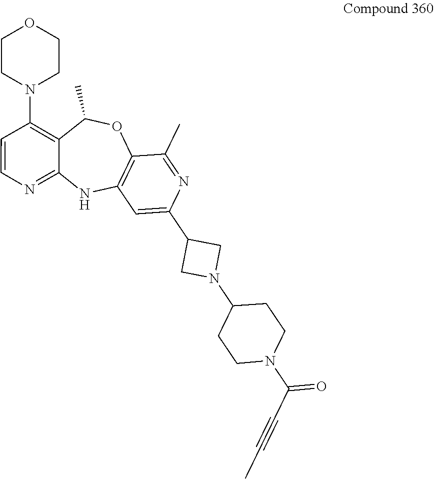 [SMILES: CC#CC(=O)N1CCC(N2CC(c3cc4c(c(C)n3)O[C@@H](C)c3c(N5CCOCC5)ccnc3N4)C2)CC1]

(*S), pure stereoisomer but absolute stereochemistry undetermined

Compound 360 was synthesized following a similar sequence of reactions as for the synthesis of Compound 358 using Intermediate 664B instead of Intermediate 664A.

LC-MS: pure (RT: 1.511, Area %: 99, [M+H]⁺: 517, Method: 2)

¹H NMR (300 MHz, DMSO-d₆) δ (ppm) 9.61 (s, 1H), 8.08 (d, J=5.2 Hz, 1H), 7.01 (s, 1H), 6.68 (d, J=5.3 Hz, 1H), 5.91 (q, J=6.9 Hz, 1H), 3.99 (d, J=13.1 Hz, 1H), 3.89 (d, J=13.7 Hz, 1H), 3.84-3.70 (m, 4H), 3.64-3.45 (m, 3H), 3.44-3.26 (m, 1H), 3.27-3.14 (m, 2H), 3.13-2.98 (n, 1H), 2.96-2.81 (m, 4H), 2.42-2.25 (m, 1H), 2.31 (s, 3H), 2.01 (s, 3H), 1.63 (t, J=16.0 Hz, 2H), 1.24 (d, J=6.7 Hz, 3H), 1.22-1.01 (m, 2H)

SFC: pure (RT: 8.708, Area %: 99, [M+H]⁺: 517, Method: LuX-Amilose_1-2-propanol 25 to 60% 30° C.)

M.P.: 220.7° C. (Mettler Toledo FP62)

O.R.: +37.5589° (589 nm, c 0.113333 w/v, DMF, 23° C.)

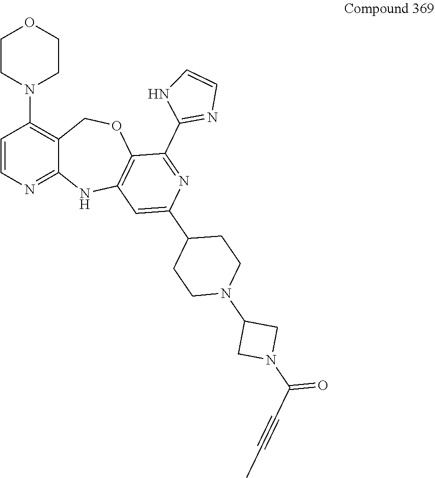 [SMILES: CC#CC(=O)N1CC(N2CCC(c3cc4c(c(-c5ncc[nH]5)n3)OCc3c(N5CCOCC5)ccnc3N4)CC2)C1]

NaBH(OAc)₃ (229 mg, 1.08 mmol, 2 eq.) was added to a solution of Intermediate 114 (522 mg, 0.54 mmol), Et₃N (225 μL, 1.62 mmol, 3 eq.), and Intermediate 112 (111 mg, 0.81 mmol, 1.5 eq.) in DCE (15 mL). The reaction mixture was stirred at room temperature overnight. Aqueous Na₂CO₃ (1 M) was added and the mixture was extracted with DCM. The organic layer was dried over MgSO₄, filtered, and evaporated. The residue was purified by reverse phase chromatography (Phenomenex Gemini C18 100×30 mm 5 μm; gradient from 72% H₂O−28% ACN−MeOH to 36% H₂O−64% ACN−MeOH, [25 mM NH₄HCO₃]). The desired fractions were collected and concentrated. The residue was triturated in E120 to give Compound 369 (77 mg, yield: 25%) as a white solid.

LCMS: confirms the MW (RT: 1.28 [M+H]⁺ 555, Method: 2).

MP: 229.2° C. (Mettler Toledo FP62), 10° C./min, uncorrected.

¹H NMR (300 MHz, DMSO-d₆) δ (ppm) 12.19 (s, 1H), 9.57 (s, 1H), 8.06 (d, J=5.1 Hz, 1H), 7.67 (s, 1H), 7.61 (s, 1H), 6.92 (s, 1H), 6.58 (d, J=5.3 Hz, 1H), 5.14 (s, 2H), 4.16 (t, J=7.9 Hz, 1H), 4.00-3.89 (m, 2H), 3.86-3.76 (m, 4H), 3.75-3.65 (m, 1H), 3.22-3.08 (m, 1H), 3.01-2.84 (m, 6H), 2.00 (s, 3H), 1.97-1.72 (m, J=24.0 Hz, 6H).

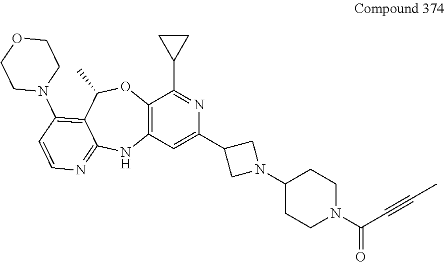 [SMILES: CC#CC(=O)N1CCC(N2CC(c3cc4c(c(C5CC5)n3)O[C@@H](C)c3c(N5CCOCC5)ccnc3N4)C2)CC1]

(*S), pure stereoisomer but absolute stereochemistry undetermined

Compound 374 was synthesized following a similar sequence of reactions as for the synthesis of Compound 376 using Intermediate 668B instead of Intermediate 668A.

LC-MS: mw confirmed (RT: 1.933, Area %: 98, [M+H]⁺: 543.2, Method: 2)

¹H NMR (300 MHz, Chloroform-d) d (ppm) 1.02 (d, J=6.5 Hz, 2H), 1.13 (q, J=5.5, 4.5 Hz, 2H), 1.21-1.27 (m, 1H), 1.38 (d, J=6.8 Hz, 3H), 1.62 (s, 3H), 1.76 (s, 1H), 2.00 (d, J=2.3 Hz, 3H), 2.35-2.60 (m, 2H), 2.82-2.96 (m, 2H), 3.07 (dt, J=9.9, 4.7 Hz, 2H), 3.32 (d, J=12.0 Hz, 3H), 3.68 (s, 3H), 3.89 (d, J=4.6 Hz, 4H), 4.20 (d, J=13.3 Hz, 2H), 6.01 (q, J=7.0 Hz, 1H), 6.36 (d, J=2.1 Hz, 1H), 6.56 (d, J=5.3 Hz, 1H), 7.14 (s, 1H), 8.06 (d, J=5.4 Hz, 1H).

M.P.: 134.7° C. (Mettler Toledo MP50)

O.R.: −13.333° (589 nm, c 0.096 w/v, DMF, 23.0° C.)

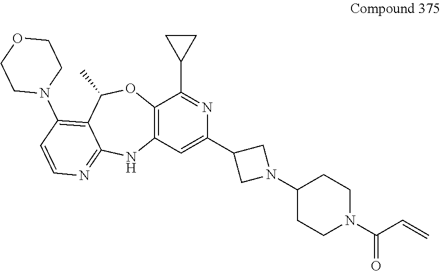 [SMILES: C=CC(=O)N1CCC(N2CC(c3cc4c(c(C5CC5)n3)O[C@@H](C)c3c(N5CCOCC5)ccnc3N4)C2)CC1]

(*S), pure stereoisomer but absolute stereochemistry undetermined

Compound 375 was synthesized following a similar sequence of reactions as for the synthesis of Compound 377 using Intermediate 668B instead of Intermediate 668A.

LC-MS: mw confirmed (RT: 1.876, Area %: 98, [M+H]⁺: 531.1, Method: 2)

¹H NMR (300 MHz, Chloroform-d) d (ppm) 1.03 (s, 2H), 1.13 (d, J=5.5 Hz, 2H), 1.25 (s, 1H), 1.37 (d, J=6.9 Hz, 3H), 1.59 (s, 3H), 1.76 (d, J=13.0 Hz, 2H), 2.38 (s, 1H), 2.44-2.59 (m, 1H), 2.92 (q, J=7.0, 5.7 Hz, 2H), 2.98-3.12 (m, 2H), 3.12-3.24 (m, 1H), 3.29 (d, J=8.6 Hz, 2H), 3.64 (s, 3H), 3.89 (t, J=4.7 Hz, 4H), 4.32 (d, J=13.2 Hz, 1H), 5.66 (dd, J=10.5, 2.1 Hz, 1H), 6.01 (q, J=6.9 Hz, 1H), 6.24 (d, J=16.9 Hz, 1H), 6.37 (s, 1H), 6.51-6.66 (m, 2H), 7.12 (s, 1H), 8.07 (s, 1H).

M.P.: 208.2° C. (Mettler Toledo MP50)

O.R.: −25.577° (589 nm, c 0.1213 w/v, DMF, 23.0° C.)

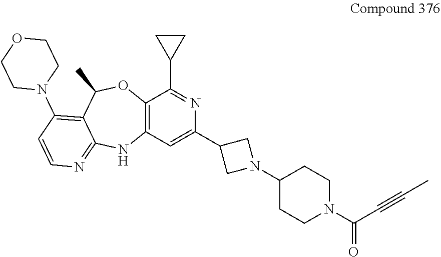 [SMILES: CC#CC(=O)N1CCC(N2CC(c3cc4c(c(C5CC5)n3)O[C@H](C)c3c(N5CCOCC5)ccnc3N4)C2)CC1]

(*R), pure stereoisomer but absolute stereochemistry undetermined

Compound 376 was prepared according to the same procedure as Compound 358, starting from Intermediate 669 instead of Intermediate 666.

LC-MS: mw confirmed (RT: 1.937, Area %: 99, [M+H]⁺: 543.2, Method: 2)

¹H NMR (300 MHz, Chloroform-d) d (ppm) 1.02 (d, J=6.8 Hz, 2H), 1.13 (d, J=6.4 MHz, 2H), 1.20-1.29 (m, 1H), 1.37 (d, J=6.9 Hz, 3H), 1.61 (s, 21H), 1.74 (s, 21H), 2.01 (s, 3H), 2.38 (s, 1H), 2.51 (tt, J=8.6, 4.8 Hz, 1H), 2.82-2.97 (m, 2H), 3.07 (dt, J=12.6, 4.3 Hz, 2H), 3.29 (d, J=10.2 Hz, 3H), 3.64 (s, 3H), 3.89 (t, J=4.6 Hz, 4H), 4.17 (d, J=11.9 Hz, 2H), 6.01 (q, J=6.9 Hz, 1H), 6.37 (d, J=2.2 Hz, 1H), 6.56 (d, J=5.5 Hz, 1H), 7.13 (s, 1H), 8.06 (d, J=5.4 Hz, 1H).

M.P.: 131.3° C. (Mettler Toledo MP50)

O.R.: +17.4009° (589 nm, c 0.1346 w/v, DMF, 23.0° C.)

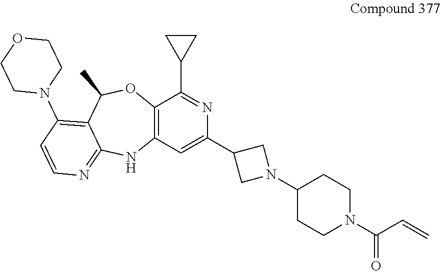 [SMILES: C=CC(=O)N1CCC(N2CC(c3cc4c(c(C5CC5)n3)O[C@H](C)c3c(N5CCOCC5)ccnc3N4)C2)CC1]

(*R), pure stereoisomer but absolute stereochemistry undetermined

Acryloyl chloride (64 μL, 0.791 mmol, 1 eq.) was added dropwise to a solution of Intermediate 669 (377 mg, 0.791 mmol) and DIPEA (690 μL, 3.953 mmol, 5 eq.) in DCM (10 mL) at 0° C. The reaction mixture was stirred at room temperature for 2 h. The reaction was quenched by addition of saturated aqueous NaHCO₃ and the mixture was extracted with DCM. The organic layer was concentrated under vacuum and the residue was purified by reverse phase chromatography (Phenomenex Gemini C18 100A column (100 m×30 mm I.D.; 5 μm particles): gradient from 70% NH₄OAc aqueous solution (65 mM+ACN 10%)/30% (ACN/MeOH 1/1) to 27% NH₄OAc aqueous solution (65 mM+ACN 10%)/73% (ACN/MeOH 1/1)) to give Compound 377 (163 mg, yield: 39%).

LC-MS: mw confirmed (RT: 1.869, Area %: 98, [M+H]: 531.2, Method: 2)

¹H NMR (300 MHz, Chloroform-d) d (ppm) 1.03 (s, 2H), 1.13 (d, J=6.8 Hz, 2H), 1.25 (s, 1H), 1.38 (d, J=6.9 Hz, 3H), 1.57 (s, 3H), 1.76 (d, J=13.0 Hz, 2H), 2.39 (s, 1H), 2.51 (s, 1H), 2.91 (d, J=12.1 Hz, 2H), 2.97-3.12 (m, 2H), 3.21 (d, J=12.0 Hz, 1H), 3.31 (s, 2H), 3.65 (s, 3H), 3.89 (t, J=4.7 Hz, 4H), 4.32 (s, 1H), 5.66 (d, J=10.5 Hz, 1H), 6.01 (q, J=6.9 Hz, 1H), 6.24 (d, J=16.8 Hz, 1H), 6.37 (s, 1H), 6.52-6.66 (m, 2H), 7.11 (s, 1H), 8.06 (d, J=5.5 Hz, 1H).

M.P.: 206.5° C. (Mettler Toledo MP50)

O.R.: +11.8651° (589 nm, c 0.084 w/v, DMF, 23.0° C.)

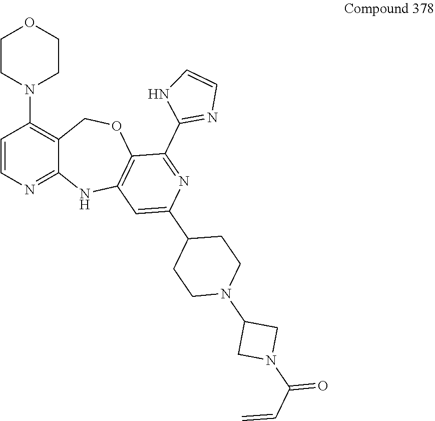 [SMILES: C=CC(=O)N1CC(N2CCC(c3cc4c(c(-c5ncc[nH]5)n3)OCc3c(N5CCOCC5)ccnc3N4)CC2)C1]

A solution of Intermediate 114 (314 mg, 0.643 mmol) and Et₃N (179 μL, 1.285 mmol, 2 eq.) in DCM (40 mL) was cooled to 0° C. under nitrogen atmosphere. A solution of acryloyl chloride (52 μL, 0.643 mmol, 1 eq.) in DCM (20 mL) was then added dropwise over 5 min. The reaction mixture was stirred for another 30 min at 0° C. Na₂CO₃ (1 M in water, 10 mL) was added with stirring. After 5 min, the organic layer was separated and aqueous layer was extracted again with DCM (50 mL). The combined organic layers were dried over MgSO₄, filtered, and concentrated. The residue was purified by reverse phase chromatography (Phenomenex Gemini C18 30×100 mm 5 μm; gradient from [72% H₂O−28% ACN−MeOH] to [36% H₂O−64% ACN−MeOH], [25 mM NH₄HCO₃]) followed by another reverse phase chromatography (Phenomenex Gemini C18 30×100 mm 5 μm; gradient from [50% H₂O−50% ACN−MeOH] to [75% H₂O−25% ACN−MeOH], [0.1% HCOOH]). The desired fractions were collected and were neutralized with solid Na₂CO₃, and extracted with DCM. The organic layer was dried over MgSO₄, filtered, and evaporated. The residue was triturated in Et₂O and filtered to give Compound 378 (35 mg).

LCMS: confirms the MW (RT: 1.24 [M+H]⁺ 543, Method: 2).

MP: 195.5° C. (Mettler Toledo FP62), 10° C./min, uncorrected.

¹H NMR (300 MHz, DMSO-d₆) δ (ppm) 9.63 (s, 1H), 8.07 (d, J=5.3 Hz, 1H), 7.78 (s, 1H), 7.67 (s, 1H), 6.95 (s, 1H), 6.59 (d, J=5.1 Hz, 1H), 6.32 (dd, J=16.9, 10.5 Hz, 1H), 6.10 (d, J=16.8 Hz, 1H), 5.67 (d, J=10.4 Hz, 1H), 5.16 (s, 2H), 4.26 (t, J=7.8 Hz, 1H), 4.11-4.01 (m, 1H), 4.01-3.91 (m, 1H), 3.89-3.70 (m, 6H), 3.22-3.12 (m, 2H), 3.01-2.88 (m, 6H), 1.87 (dd, J=37.4, 16.7 Hz, 6H)

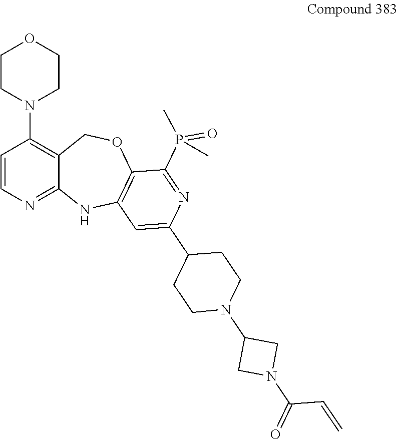 [SMILES: C=CC(=O)N1CC(N2CCC(c3cc4c(c(P(C)(C)=O)n3)OCc3c(N5CCOCC5)ccnc3N4)CC2)C1]

Intermediate 124 (60 mg, 0.12 mmol) was dissolved in a mixture of Et₃N (84 μL, 0.602 mmol, 5 eq.) and DCM (12 mL). The mixture was cooled to 0° C. Acryloyl chloride (13 μL, 0.156 mmol, 1.3 eq.) was then added and the reaction mixture was stirred for 30 min. The volatiles were evaporated and the residue was dissolved in DCM and washed with Na₂CO₃ (1 M in water, 10 mL). The organic layer was dried over MgSO₄, filtered, and concentrated to dryness. The residue was purified by reverse phase chromatography (Phenomenex Gemini C18 100×30 mm 5 μm; gradient from [81% H₂O−19% ACN−MeOH] to [45% H₂O−55% ACN−MeOH], [25 mM NH₄HCO₃]) to give Compound 383 (11 mg, yield: 16%).

LCMS: confirms the MW (RT: 1.41, [M+H]⁺ 553, Method: 3).

¹H NMR (300 MHz, CDCl₃) δ (ppm) 8.05 (d, J=5.4 Hz, 1H), 7.32 (s, 1H), 6.67 (s, 1H), 6.51 (d, J=5.4 Hz, 1H), 6.32 (d, J=16.8 Hz, 1H), 6.19 (dd, J=16.9, 10.1 Hz, 1H), 5.66 (d, J=10.1 Hz, 1H), 5.19 (s, 2H), 4.24 (t, J=7.8 Hz, 1H), 4.17-4.06 (m, 2H), 4.03-3.92 (m, 1H), 3.87 (s, 4H), 3.29-3.13 (m, 1H), 2.97 (s, 6H), 2.70-2.54 (m, 1H), 2.08-1.72 (m, 6H), 1.86 (d, J=13.4 Hz, 6H)

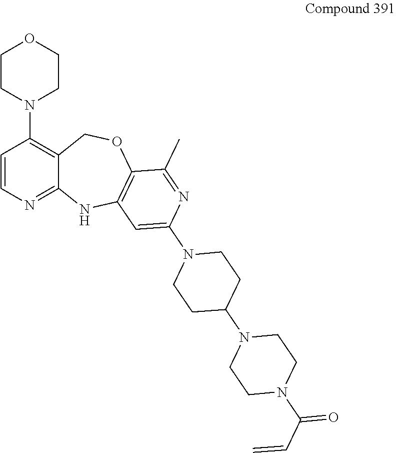 [SMILES: C=CC(=O)N1CCN(C2CCN(c3cc4c(c(C)n3)OCc3c(N5CCOCC5)ccnc3N4)CC2)CC1]

A solution of Intermediate 676 (252 mg, 0.514 mmol) and Et₃N (143 μL, 1.028 mmol, 2 eq.) in DCM (10 mL) was cooled to 0° C. under nitrogen atmosphere. A solution of acryloyl chloride (50 μL, 0.617 mmol, 1.2 eq.) in DCM (5 ml) was then added dropwise over 5 min. The reaction mixture was stirred for another 30 min at 0′ C. Aqueous Na₂CO₃ (1 M, 10 mL) was added with stirring. After 5 min, the layers were separated and the aqueous layer was extracted with DCM (50 mL). The combined organic layers were dried over MgSO₄, filtered, and concentrated. The residue was purified by reverse phase chromatography (Phenomenex Gemini C18 100×30 mm 5 μm; gradient from 72% H₂O−28% ACN−MeOH to 36% H₂O−64% ACN−MeOH)−[25 mM NH₄HCO₃]. The desired fractions were evaporated and the residue was triturated in Et₂O to give Compound 391 (151 ng, yield: 56%) as a solid.

LC-MS: pure (RT: 1.334, Area %: 99, [M+H]⁺: 520, Method: 2)

¹H NMR (300 MHz, DMSO-d₆) δ (ppm) 9.19 (s, 1H), 8.01 (d, J=5.4 Hz, 1H), 6.78 (dd, J=16.7, 10.4 Hz, 1H), 6.53 (d, J=5.4 Hz, 1H), 6.44 (s, 1H), 6.09 (d, J=16.7 Hz, 1H), 5.66 (d, J=10.5 Hz, 1H), 4.94 (s, 2H), 4.08 (d, J=12.3 Hz, 2H), 3.76 (s, 4H), 3.51 (s, 4H), 2.87 (s, 4H), 2.62 (t, J=12.1 Hz, 2H), 2.52-2.43 (m, 4H), 2.45-2.31 (m, 1H), 2.22 (s, 31H), 1.79 (d, J=11.3 Hz, 2H), 1.49-1.28 (m, 2H)

M.P.: 225.4° C. (Mettler Toledo FP62)

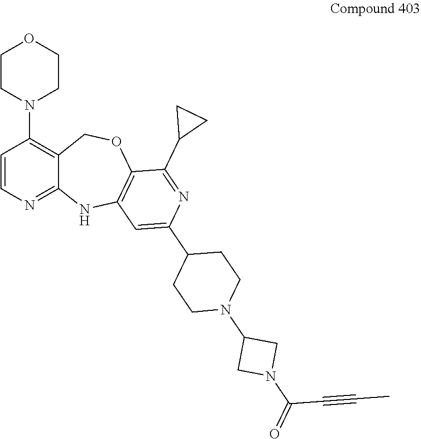 [SMILES: CC#CC(=O)N1CC(N2CCC(c3cc4c(c(C5CC5)n3)OCc3c(N5CCOCC5)ccnc3N4)CC2)C1]

HBTU (580 mg, 1.529 mmol, 1.33 eq.) was added to a solution of Intermediate 89 (1052 mg, 1.145 mmol), DIPEA (974 μL, 5.726 mmol, 5 eq.), and 2-butynoic acid (144 mg, 1.718 mmol, 1.5 eq.) in DCM (25 mL). The reaction mixture was stirred at room temperature for 2 h. Na₂CO₃ (1 M in water) was added and the mixture was extracted with DCM. The organic layer was dried over MgSO₄, filtered, and concentrated. The residue was purified by column chromatography over silica gel (DCM:MeOH (9:1) in DCM from 0% to 100%) followed by reverse phase chromatography (Phenomenex Gemini C18 100×30 mm 5 μm; gradient from [59% H₂O−41% ACN−MeOH] to [17% H₂O−83% ACN−MeOH], [25 mM NH₄HCO₃]). The obtained solid was triturated in ACN to give Compound 403 (105 mg, yield: 17%) as an off-white solid.

LCMS: confirms the MW (RT: 1.57, [M+H]⁺ 529, Method: 2).

MP: 174.8° C. (METTLER Toledo FP62), 10° C./min uncorrected.

¹H NMR (300 MHz, DMSO-d₆) δ (ppm) 9.38 (s, 1H), 8.04 (d, J=5.4 Hz, 1H), 6.78 (s, 1H), 6.55 (d, J=5.5 Hz, 1H), 5.05 (s, 2H), 4.13 (t, J=7.9 Hz, 1H), 3.97-3.85 (m, 2H), 3.83-3.74 (m, 4H), 3.73-3.64 (m, 1H), 3.19-3.05 (m, 1H), 2.97-2.88 (m, 4H), 2.84 (d, J=10.7 Hz, 2H), 2.48-2.42 (m, 1H), 2.34 (t, J=11.0 Hz, 1H), 1.99 (s, 3H), 1.86 (t, J=10.8 Hz, 2H), 1.79-1.55 (m, J=34.9, 11.2 Hz, 4H), 0.93-0.79 (m, J=7.9 Hz, 4H)

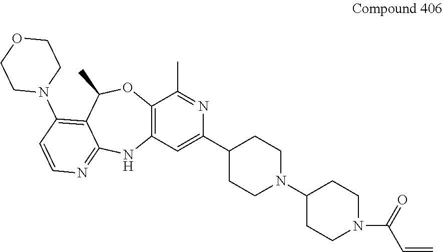 [SMILES: C=CC(=O)N1CCC(N2CCC(c3cc4c(c(C)n3)O[C@H](C)c3c(N5CCOCC5)ccnc3N4)CC2)CC1]

(*R), pure stereoisomer but absolute stereochemistry undetermined

DIPEA (1.675 mL, 9.59 mmol, 5 eq.) was added to a solution of Intermediate 685 (918 mg, 1.918 mmol) in DCM (25 mL). The reaction mixture was cooled to 0° C. and acryloyl chloride (155 μL, 1.918 mmol, 1 eq.) was added dropwise. The reaction mixture was then stirred at room temperature for 3 h. The reaction was quenched by addition of saturated aqueous NaHCO₃ and the mixture was extracted with DCM. The organic layer was concentrated under vacuum and the residue was purified by reverse phase column chromatography (Phenomenex Gemini C18 100A column (100 mm×30 mm I.D.; 5 μm particles; gradient from 72% of a NH₄CO₃ aqueous solution (25 mM+ACN 10%)/28% (ACN/MeOH 1/1) to 36% of a NH₄CO₃ aqueous solution (25 mM+ACN 10%)/64% (ACN/MeOH 1/1)) to give Compound 406 (308 mg, yield: 30%) as a white solid.

LC-MS: MW confirmed (RT: 1.366, Area %: 99, [M+H]⁺: 5331, Method: 2)

¹H NMR (300 MHz, Chloroform-d) d (ppm) 1.35 (d, J=6.9 Hz, 3H), 1.41-1.59 (m, 2H), 1.65-1.80 (m, 2H), 1.95 (t, J=14.5 Hz, 4H), 2.30 (t, J=11.4 Hz, 2H), 2.43 (s, 3H), 2.48-2.73 (m, 3H), 2.82-2.98 (m, 2H), 2.97-3.14 (m, 5H), 3.90 (dt, J=5.5, 2.6 Hz, 4H), 4.06 (d, J=13.7 Hz, 1H), 4.71 (d, J=13.1 Hz, 1H), 5.67 (dd, J=10.5, 2.1 Hz, TH), 5.95 (q, J=7.0 Hz, 1H), 6.26 (dd, J=16.9, 2.1 Hz, 1H), 6.39 (s, 1H), 6.52-6.65 (m, 2H), 7.11 (s, 1H), 8.06 (d, J=5.5 Hz, 1H).

M.P.: 253.4° C. (Mettler Toledo MP50)

O.R.: +50.990 (c 0.1067 w/v, DMF, 23° C.)

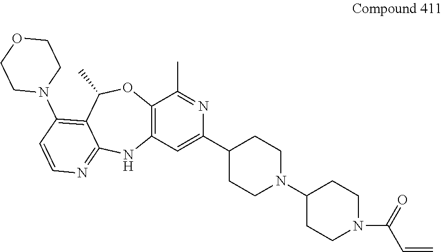 [SMILES: C=CC(=O)N1CCC(N2CCC(c3cc4c(c(C)n3)O[C@@H](C)c3c(N5CCOCC5)ccnc3N4)CC2)CC1]

(*S), pure stereoisomer but absolute stereochemistry undetermined

Compound 411 was synthesized following a similar sequence of reactions as for the synthesis of Compound 406 using Intermediate 686 instead of Intermediate 685.

LC-MS: MW confirmed (RT: 1.344, Area %: 99, [M+H]⁺: 533.2, Method: 2)

¹H NMR (300 MHz, Chloroform-d) d (ppm) 1.35 (d, J=6.9 Hz, 3H), 1.41-1.59 (m, 2H), 1.65-1.80 (m, 2H), 1.95 (t, J=14.5 Hz, 4H), 2.30 (t, J=11.4 Hz, 2H), 2.43 (s, 3H), 2.48-2.73 (m, 3H), 2.82-2.98 (m, 2H), 2.97-3.14 (m, 5H), 3.90 (dt, J=5.5, 2.6 Hz, 4H), 4.06 (d, J=13.7 Hz, 1H), 4.71 (d, J=13.1 Hz, 1H), 5.67 (dd, J=10.5, 2.1 Hz, 1H), 5.95 (q, J=7.0 Hz, 1H), 6.26 (dd, J=16.9, 2.1 Hz, 1H), 6.39 (s, 1H), 6.52-6.65 (m, 2H), 7.11 (s, 1H), 8.06 (d, J=5.51 z, 1H).

M.P.: 255.1° C. (Mettler Toledo MP50)

O.R.: −49.13° (c 0.1227 w/v, DMF, 23° C.)

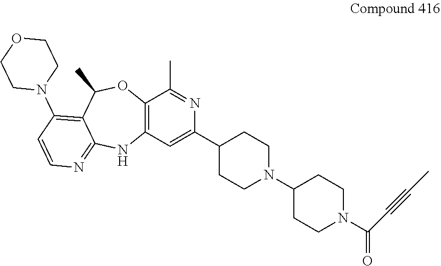 [SMILES: CC#CC(=O)N1CCC(N2CCC(c3cc4c(c(C)n3)O[C@H](C)c3c(N5CCOCC5)ccnc3N4)CC2)CC1]

(*R), pure stereoisomer but absolute stereochemistry undetermined

Compound 416 was prepared according to the same procedure as Compound 358, starting from Intermediate 685 instead of Intermediate 666.

LC-MS: MW confirmed (R-T: 1.404, Area %: 99, [M+H]⁺: 545.2, Method: 2)

¹H NMR (300 MHz, Chloroform-cd) d (ppm) 1.35 (d, J=6.9 Hz, 31H), 1.41-1.58 (in, 24), 1.66-1.80 (m, 2H), 1.83-1.99 (m, 2H), 2.01 (s, 5H), 2.31 (t, J=11.7 Hz, 2H), 2.43 (s, 3H), 2.49-2.71 (m, 3H), 2.91 (dt, J=11.9, 4.8 Hz, 2H), 3.05 (ddt, J=15.2, 10.9, 6.0 Hz, 5H), 3.82-3.99 (m, 4H), 4.43 (d, J=13.5 Hz, 1H), 4.60 (d, J=13.4 Hz, 1H), 5.95 (q, J=6.9 Hz, 1H), 6.39 (s, 1H), 6.55 (d, J=5.4 Hz, 1H), 7.12 (s, 1H), 8.06 (d, J=5.5 Hz, 1H).

M.P.: 163° C. (Mettler Toledo MP50)

O.R.: +18.57 (589 nm, c 0.1153 w/v: DMF, 23° C.

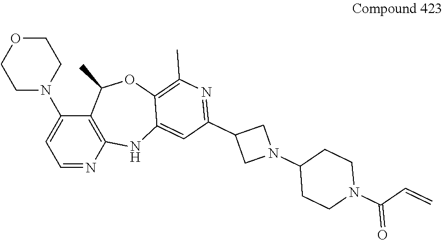 [SMILES: C=CC(=O)N1CCC(N2CC(c3cc4c(c(C)n3)O[C@H](C)c3c(N5CCOCC5)ccnc3N4)C2)CC1]

(*R), pure stereoisomer but absolute stereochemistry undetermined

Compound 423 was prepared according to the same procedure as Compound 391, starting from Intermediate 666 instead of Intermediate 676.

LC-MS: Pure (RT: 1.419, Area %: 99, [M+H]⁺: 505.2, Method: 2)

¹H NMR (300 MHz, DMSO-d₆) δ (ppm) 9.60 (s, 1H), 8.08 (d, J=5.3 Hz, 1H), 7.02 (s, 1H), 6.79 (dd, J=16.7, 10.5 Hz, 1H), 6.68 (d, J=5.3 Hz, 1H), 6.06 (d, J=16.8 Hz, 1H), 5.90 (q, J=6.6 Hz, 1H), 5.64 (d, J=10.5 Hz, 1H), 4.06-3.93 (m, 1H), 3.89-3.80 (m, 1H), 3.80-3.73 (m, 4H), 3.57-3.42 (m, 3H), 3.29-3.12 (m, 3H), 3.06 (t, J=10.1 Hz, 1H), 2.94-2.84 (m, 4H), 2.36-2.26 (m, 1H), 2.31 (s, 3H), 1.72-1.57 (m, 2H), 1.24 (d, J=6.7 Hz, 3H), 1.21-1.01 (m, 2H)

M.P.: 207.7° C. (Mettler Toledo FP62)

O.R.: +48.58860 (589 nm, c 0.1393 w/v MeOH, 23° C.)

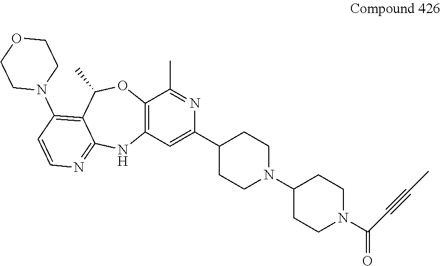 [SMILES: CC#CC(=O)N1CCC(N2CCC(c3cc4c(c(C)n3)O[C@@H](C)c3c(N5CCOCC5)ccnc3N4)CC2)CC1]

(*S), pure stereoisomer but absolute stereochemistry undetermined

Compound 426 was prepared according to the same procedure as Compound 358, starting from Intermediate 686 instead of Intermediate 666.

LC-MS: MW confirmed (RT: 1.415, Area %: 99, [M+H]⁺: 545.2, Method: 2)

¹H NMR (300 MHz, Chloroform-d) d (ppm) 1.35 (d, J=6.9 Hz, 31H), 1.41-1.58 (m, 2H), 1.66-1.80 (m, 2H), 1.83-1.99 (m, 2H), 2.01 (s, 5H), 2.31 (t, J=11.7 Hz, 2H), 2.43 (s, 3H), 2.49-2.71 (m, 3H), 2.91 (dt, J=11.9, 4.8 Hz, 2H), 3.05 (ddt, J=15.2, 10.9, 6.0 Hz, 5H), 3.82-3.99 (m, 4H), 4.43 (d, J=13.5 Hz, 1H), 4.60 (d, J=13.4 Hz, 1H), 5.95 (q, J=6.9 Hz, 1H), 6.39 (s, 1H), 6.55 (d, J=5.4 Hz, 1H), 7.12 (s, 1H), 8.06 (d, J=5.5 Hz, 1H).

M.P.: 223.3° C. (Mettler Toledo MP50)

O.R.: −13.19° (589 nm, c 0.0867 w/v, DMF, 23° C.)

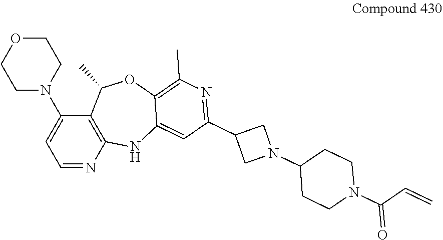 [SMILES: C=CC(=O)N1CCC(N2CC(c3cc4c(c(C)n3)O[C@@H](C)c3c(N5CCOCC5)ccnc3N4)C2)CC1]

(*S), pure stereoisomer but absolute stereochemistry undetermined

Compound 430 was synthesized following a similar sequence of reactions as for the synthesis of Compound 423 using Intermediate 664B instead of Intermediate 664A.

LC-MS: Pure (RT: 1.394, Area %: 99, [M+H]⁺: 505.2, Method:)

¹H NMR (300 MHz, DMSO-d₆) δ (ppm) 9.60 (s, 1H), 8.08 (d, J=5.3 Hz, 1H), 7.02 (s, 1H), 6.79 (dd, J=16.6, 10.5 Hz, 1H), 6.68 (d, J=5.4 Hz, 1H), 6.06 (d, J=16.7 Hz, 1H), 5.91 (q, J=6.6 Hz, TH), 5.64 (d, J=10.4 Hz, 1H), 4.08-3.93 (m, 1H), 3.93-3.72 (m, 5H), 3.60-3.44 (m, 3H), 3.27-3.12 (m, 3H), 3.06 (t, J=9.8 Hz, 1H), 2.95-2.84 (n, 4H), 2.41-2.23 (m, 1H), 2.31 (s, 3H), 1.73-1.57 (m, 2H), 1.24 (d, J=6.7 Hz, 3H), 1.19-1.04 (m, 2H).

M.P.: 205.7° C. (Mettler Toledo FP62)

O.R.: −52.1759° (589 nm, c 0.144 w/v, MeOH, 23° C.)

### Compound 443 and Compound 442

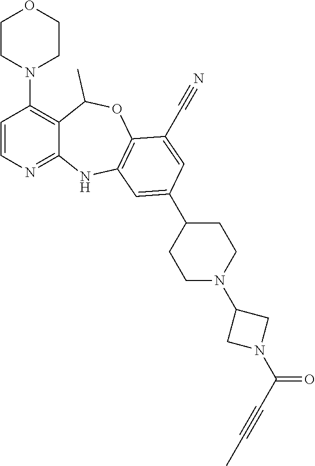 [SMILES: CC#CC(=O)N1CC(N2CCC(c3cc(C#N)c4c(c3)Nc3nccc(N5CCOCC5)c3C(C)O4)CC2)C1] [1925] Compound 443: (*R), pure stereoisomer but absolute stereochemistry undetermined
Compound 442: (*S), pure stereoisomer but absolute stereochemistry undetermined

Intermediate 689 (203 mg, 1.48 mmol, 2 eq.) was added to a solution of Intermediate 688 (300 mg, 0.74 mmol) and AcOH (85 μL, 1.48 mmol, 2 eq.) in DCE (6.4 mL) at room temperature. The reaction mixture was stirred at room temperature for 30 min. Sodium triacetoxyborohydride (314 mg, 1.48 mmol, 2 eq.) was added and the reaction mixture was stirred at room temperature for 18 h. The reaction mixture was poured into aqueous K₂CO₃ (10%) and DCM. The mixture was filtered through Chromabond® and the filtrate was evaporated. The residue was purified by chromatography over silica gel (12 g, eluent: 99% DCM, 1% MeOH, 0.1% NH₄OH to 95% DCM, 5% MeOH, 0.5% NH₄OH) followed by chiral SFC (CHIRALPAK AS-H 5 μm 250*20 mm, mobile phase: 60% CO₂, 40% MeOH (0.6% Et₃N)). After evaporation, the pure fractions were freeze-dried with water-ACN to give Compound 443 (152 mg, yield: 39%) and Compound 442 (164 mg, yield: 42%), both as pale brown solids.

### Compound 443

LC-MS: confirms the MW, OK Plat 97% (RT: 526.30, 526.30, MW: 526.30, BPM2: 526.30, 526.30, Method: 1)

¹H NMR (DMSO-d₆, 500 MHz) δ (ppm) 9.49 (br s, 1H), 8.1-8.1 (m, 1H), 7.43 (br s, 1H), 7.07 (br s, 1H), 6.6-6.7 (m, 1H), 5.97 (br d, 1H, J=6.9 Hz), 4.15 (br t, 1H, J=7.6 Hz), 3.9-4.0 (m, 2H), 3.7-3.8 (m, 5H), 3.3-3.4 (m, 1H), 3.27 (br s, 2H), 3.17 (br s, 1H), 2.8-3.0 (m, 6H), 2.4-2.5 (m, 2H), 1.9-2.0 (m, 5H), 1.77 (br d, 2H, J=11.3 Hz), 1.59 (br d, 2H, J=11.3 Hz), 1.34 (br d, 3H, J=6.6 Hz)

SFC: RT: 3.09, 100%, [M+H] 527, Method: 7

O.R.: +55.66° (589 nm, c 0.212 w/v %, DMF, 20° C.)

### Compound 442

LC-MS: confirms the MW, OK P3 at 97%+M=501 (2%) (RT: 526.30, 526.30, MW: 526.30, BPM2: 526.30, Method: 1)

¹H NMR (DMSO-d₆, 500 MHz) d (ppm) 9.4-9.6 (m, 1H), 8.08 (br d, 1H, J=4.4 Hz), 7.42 (br s, 1H), 7.06 (br s, 1H), 6.66 (br d, 1H, J=4.4 Hz), 5.97 (br d, 1H, J=6.6 Hz), 4.15 (br t, 1H, J=7.3 Hz), 3.9-4.0 (m, 31H), 3.7-3.8 (m, 6H), 3.3-3.4 (m, 1H), 3.1-3.3 (m, 2H), 2.90 (br s, 7H), 2.46 (br d, 2H, J=12.6 Hz), 2.0-2.0 (m, 4H), 1.90 (br t, 2H, J=10.7 Hz), 1.77 (br d, 3H, J=11.3 Hz), 1.59 (br d, 3H, J=12.0 Hz), 1.33 (br d, 4H, J=6.3 Hz)

SFC: RT: 3.99, 98%, [M+H]⁺ 527, Method: 7

O.R.: −59.38° (589 nm, c 0.256 w/v %, DMF, 20° C.)

### Compound 462 and Compound 459

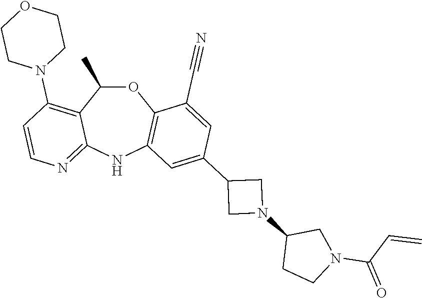 [SMILES: C=CC(=O)N1CC[C@@H](N2CC(c3cc(C#N)c4c(c3)Nc3nccc(N5CCOCC5)c3[C@@H](C)O4)C2)C1] [1937] Compound 462 (*R, *R), pure isomer but absolute stereochemistry undetermined

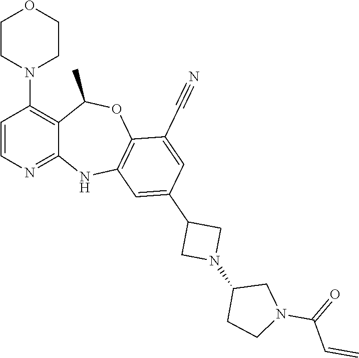 [SMILES: C=CC(=O)N1CC[C@H](N2CC(c3cc(C#N)c4c(c3)Nc3nccc(N5CCOCC5)c3[C@@H](C)O4)C2)C1]

Compound 459 (*R, *S), pure isomer but absolute stereochemistry undetermined

A solution of Intermediate 695 (149 mg, 0.26 mmol) in THF (2.9 mL) was cooled down to 0-5° C. under nitrogen. Sodium tert-butoxide 2 M in THF [CAS: 865-48-5](269 μL; 0.54 mmol) was added slowly. The reaction mixture was stirred for 30 min at 0-5° C., then the mixture was poured into a 10% aqueous solution of NH₄Cl and DCM was added. The mixture was filtered through Chromabond® and the filtrate was concentrated to dryness. Purification by chromatography over silica gel (Biotage, 12 g, eluent: from 98% DCM, 2% MeOH, 0.2% NH₄OH to 95% DCM, 5% MeOH, 0.5% NH₄OH), followed by chiral SFC (Whelk-O1 (S,S) 5 μm 250*30 mm, mobile phase: 55% CO₂, 45% MeOH (0.6% Et₃N)) afforded Compound 462 (41 mg, yield: 32%) and Compound 459 (43 mg, yield: 33%)

### Data for Compound 462:

LC MS: confirms the MW (RT: 2.41, [M+H]⁺: 500, Method: 1)

¹H NMR (500 MHz, DMSO-d₆, 30° C.): δ (ppm) 9.55 (br d, J=5.7 Hz, 1H), 8.09 (d, J 5.4 Hz, 1H), 7.53 (s, 1H), 7.15 (d, J=1.9 Hz, 1H), 6.67 (d, J=5.4 Hz, 1H), 6.57 (ddd, J=16.9, 10.2, 6.9 Hz, 1H), 6.12 (dt, J=16.9, 2.6 Hz, 1H), 5.97 (q, J=6.6 Hz, 1H), 5.62-5.68 (m, 1H), 3.79 (br t, J=4.6 Hz, 4H), 3.36-3.64 (m, 7H), 2.97-3.11 (m, 3H), 2.85-2.94 (m, 4H), 1.64-1.94 (m, 2H), 1.33 ppm (d, J=6.9 Hz, 3H)

OR: +59.92° (589 nm, c 0.257 w/v, DMF, 20.0° C.).

SFC: RT: 2.40, 100%, [M+H]⁺ 501, Method: 8

### Data for Compound 459:

LC MS: confirms the MW (RT: 2.41, [M+H]⁺: 500, Method: 1)

¹H NMR (500 MHz, DMSO-d₆, 30° C.): δ (ppm) 9.55 (br d, J=5.7 Hz, 1H), 8.09 (d, J=5.7 Hz, 1H), 7.53 (s, 1H), 7.15 (s, 1H), 6.67 (d, J=5.4 Hz, 1H), 6.57 (ddd, J=16.9, 10.2, 6.9 Hz, 1H), 6.12 (dt, J=16.9, 2.6 Hz, 1H), 5.97 (q, J=6.9 Hz, 1H), 5.62-5.68 (m, 1H), 3.79 (t, J=4.4 Hz, 4H), 3.55-3.64 (m, 3H), 3.42-3.55 (m, 2H), 3.35-3.41 (m, 1H), 3.20-3.28 (m, 2H), 2.97-3.12 (m, 3H), 2.85-2.94 (m, 4H), 1.64-1.93 (m, 2H), 1.33 ppm (d, J=6.9 Hz, 3H)

OR: +53.760 (589 nm, c 0.266 w/v, DMF, 20.0° C.).

SFC: RT: 2.85, 100% [M+H]⁺ 501, Method: 8

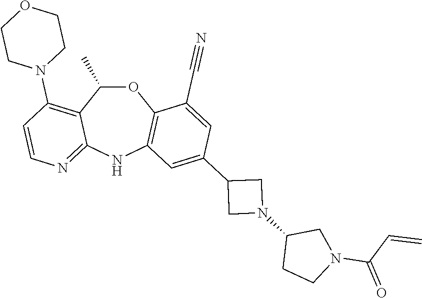 [SMILES: C=CC(=O)N1CC[C@H](N2CC(c3cc(C#N)c4c(c3)Nc3nccc(N5CCOCC5)c3[C@H](C)O4)C2)C1]

### Compound 460 and Compound 460

Compound 460 (*S, *S), pure isomer but absolute stereochemistry undetermined

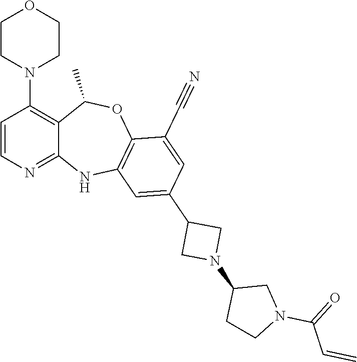 [SMILES: C=CC(=O)N1CC[C@@H](N2CC(c3cc(C#N)c4c(c3)Nc3nccc(N5CCOCC5)c3[C@H](C)O4)C2)C1]

Compound 461 (*S, *R), pure isomer but absolute stereochemistry undetermined

Compound 460 and Compound 461 were synthesized following a similar sequence of reactions as for the synthesis of Compound 459 and Compound 462 using Intermediate 694B instead of Intermediate 694A and purified by SFC (Whelk-O1 (S,S) 5 μm 250 * 21.2 mm, mobile phase: 55% CO₂, 45% MeOH (0.3% iPrNH₂)).

### Data for Compound 460:

LC MS: confirms the MW (RT: 2.42, [M+H]⁺: 500, Method: 1)

¹H NMR (500 MHz, DMSO-d₆, 30° C.): δ (ppm) 9.55 (br d, J=6.0 Hz, 1H), 8.09 (d, J=5.4 Hz, 1H), 7.53 (s, 1H), 7.15 (d, J=1.9 Hz, 1H), 6.67 (d, J=5.4 Hz, 1H), 6.57 (ddd, J=16.8, 10.2, 6.8 Hz, 1H), 6.12 (dt, J=16.7, 2.7 Hz, 1H), 5.97 (q, J=6.9 Hz, 1H), 5.60-5.68 (m, 1H), 3.79 (t, J=4.4 Hz, 4H), 3.43-3.62 (m, 5H), 3.32-3.41 (m, 1H), 3.21-3.27 (m, 1H), 2.98-3.16 (m, 3H), 2.83-2.96 (m, 4H), 1.62-1.91 (m, 2H), 1.33 ppm (d, J=6.9 Hz, 3H)

OR: −60.69° (589 nm, c 0.262 w/v, DMF, 20.0° C.).

SFC: RT: 2.80, 100%, [M+H]⁺ 501, Method: 8

### Data for Compound 461:

LC MS: confirms the MW (RT: 2.41, [M+H]⁺: 500, Method: 1)

¹H NMR (500 MHz, DMSO-d₆, 30° C.): δ (ppm) 9.55 (br d, J=5.4 Hz, 1H), 8.09 (d, J=5.4 Hz, 1H), 7.52 (s, 1H), 7.15 (s, 1H), 6.67 (d, J=5.7 Hz, 1H), 6.53-6.60 (m, 1H), 6.09-6.15 (m, 1H), 5.97 (q, J=6.9 Hz, 1H), 5.62-5.67 (m, 1H), 3.79 (t, J=4.4 Hz, 4H), 3.36-3.63 (m, 6H), 3.20-3.27 (m, 1H), 3.03-3.11 (m, 2H), 3.01 (br s, 1H), 2.82-2.97 (m, 4H), 1.64-1.93 (m, 2H), 1.33 ppm (d, J=6.6 Hz, 3H)

OR: −52.85° (589 nm, c 0.246 w/v. DMF, 20.0° C.).

SFC: RT: 2.36, 100%, MW: [M+H]⁺ 501, Method: 8Compound 481

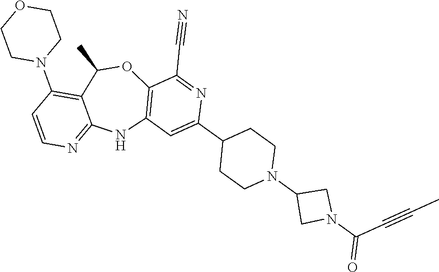 [SMILES: CC#CC(=O)N1CC(N2CCC(c3cc4c(c(C#N)n3)O[C@H](C)c3c(N5CCOCC5)ccnc3N4)CC2)C1]

(*R), pure enantiomer but absolute stereochemistry undetermined

A solution of Intermediate 133 (210 mg, 0.455 mmol) in DCM (10 mL) was added to a mixture of 2-butynoic acid (48 mg, 0.571 mmol, 1.25 eq.), HBTU (243 mg, 0.637 mmol, 1.4 eq.), and DIPEA (0.4 mL, 2.352 mmol, 5.2 eq.) in DCM (3 mL) at 0° C. under nitrogen atmosphere. The reaction mixture was stirred for 1 h at 0° C. This mixture was poured into water and NH₄Cl (10% in water). This mixture was extracted with DCM and the organic layer was filtered on Chromabond® and evaporated. The residue was purified by preparative column chromatography (Irregular SiOH 15-40 μm 40 g GraceResolv®, mobile phase Gradient from: 100% DCM to 94% DCM, 6% MeOH, 0.6% NH₄OH) followed be reverse phase column chromatography (Stationary phase: YMC-actus Triart C18 10 μm 30*150 mm, Mobile phase: Gradient from 65% NH₄HCO₃ 0.2%, 35% ACN to 35% NH₄HCO₃ 0.2%, 65% ACN). The obtained product was purified again by chiral SFC (Stationary phase: CHIRALPAK AS-H 5 μm 250*20 mm, Mobile phase: 65% CO₂, 35% MeOH (0.6% Et₃N)). The obtained product was taken up into ACN/water (2/5 mL) and freeze-dried overnight to give Compound 481 (41 mg, yield: 17%).

LCMS: confirms the MW (RT: 2.45, [M+H]⁺ 528, Method: 1).

SFC: RT: 2.27, [M+H]⁺ 528, Method: 2

¹H NMR (DMSO-d₆, 500 MHz) δ (ppm) 10.14 (br s, 1H), 8.16 (d, 1H, J=5.4 Hz), 7.31 (s, 1H), 6.79 (d, 1H, J=5.4 Hz), 6.02 (q, 1H, J=6.9 Hz), 4.15 (br t, 1H, J=8.0 Hz), 3.9-4.0 (m, 2H), 3.80 (br t, 4H, J=4.4 Hz), 3.71 (br dd, 1H, J=5.0, 10.1 Hz), 3.1-3.2 (m, 3H), 2.8-3.0 (m, 6H), 2.00 (s, 3H), 1.93 (br t, 2H, J=11.3 Hz), 1.83 (br d, 2H, J=12.3 Hz), 1.6-1.7 (m, 2H), 1.35 (d, 3H, J=6.9 Hz)

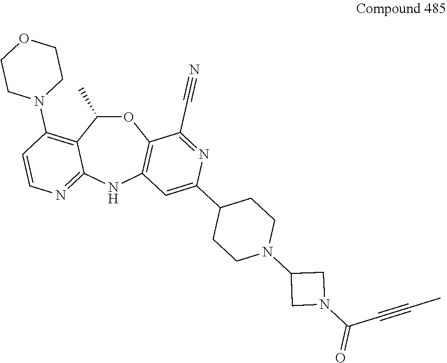 [SMILES: CC#CC(=O)N1CC(N2CCC(c3cc4c(c(C#N)n3)O[C@@H](C)c3c(N5CCOCC5)ccnc3N4)CC2)C1]

(*S), pure enantiomer but absolute stereochemistry undetermined

Compound 485 was prepared using a procedure analoguous to Compound 481, starting from Intermediate 134 instead of Intermediate 133.

LCMS: confirms the MW (RT: 2.45, [M+H]⁺ 528, Method: 1).

SFC: RT: 2.45, [M+H]⁺ 528, Method: 2

¹H NMR (DMSO-d₆, 500 MHz) δ (ppm) 10.14 (br s, 1H), 8.16 (d, 1H, J=5.4 Hz), 7.31 (s, 1H), 6.79 (d, 1H, J=5.4 Hz), 6.02 (q, 1H, J=6.9 Hz), 4.15 (t, 1H, J=8.2 Hz), 3.9-4.0 (m, 2H), 3.7-3.8 (m, 5H), 3.1-3.2 (m, 1H), 2.8-3.0 (m, 6H), 2.5-2.6 (m, 1H), 2.00 (s, 3H), 1.93 (br t, 2H, J=11.5 Hz), 1.83 (br d, 2H, J=11.7 Hz), 1.6-1.7 (m, 2H), 1.35 (d, 3H, J=6.6 Hz)

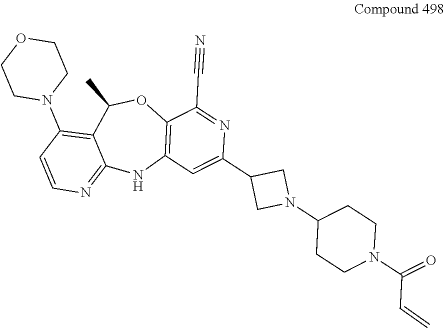 [SMILES: C=CC(=O)N1CCC(N2CC(c3cc4c(c(C#N)n3)O[C@H](C)c3c(N5CCOCC5)ccnc3N4)C2)CC1]

(*R), pure isomer, absolute stereochemistry undetermined

To a solution of Intermediate 707 (88 mg, 0.19 mmol) in DCM (10 mL), DIPEA (167 μL, 0.996 mmol) was added. The mixture was cooled in an ice bath, then a solution of acryloyl chloride [CAS: 814-68-6] (15 μL, 0.19 mmol) in DCM (150 μL) was added dropwise to the mixture and stirred at room temperature for 2 h. The reaction mixture was quenched by addition of saturated aqueous NaHCO₃ and extracted with DCM. The organic layer was separated, dried, filtered, and concentrated under vacuum. Purification by reverse phase chromatography (Phenomenex Gemini C18 30×100 mm 5 μm; gradient from 72% [25 mM NH₄HCO₃]−28% [ACN:MeOH (1:1)] to 36% [25 mM NH₄HCO₃]−64% [ACN:MeOH (1:1)) afforded Compound 489 (66 mg, yield: 67%).

LC MS: confirms the MW (RT: 1.886, [M+H]⁺: 516, Method: 2)

¹H NMR (300 MHz, Chloroform-d) d (ppm) 1.46 (d, J=6.9 Hz, 3H), 1.71-1.91 (m, 4H), 2.69 (s, 1H), 2.91 (d, J=11.7 Hz, 3H), 3.03-3.28 (m, 3H), 3.58 (s, 2H), 3.89 (h, J=7.6, 6.1 Hz, 8H), 4.43 (s, 1H), 5.68 (d, J=10.6 Hz, 1H), 6.07 (q, J=6.9 Hz, 1H), 6.26 (d, J=16.8 Hz, 1H), 6.49-6.67 (m, 2H), 6.86 (s, 1H) 7.52 (s, 1H), 8.11 (d, J=5.5 Hz, 1H).

MP: 191.5° C. (Mettler Toledo MP50), uncorrected.

OR: +54.25° (589 nm, c 0.09333w/v, DMF, 23.0° C.).

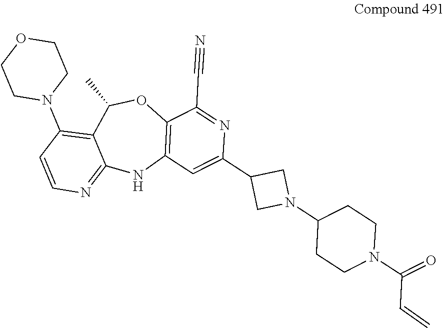 [SMILES: C=CC(=O)N1CCC(N2CC(c3cc4c(c(C#N)n3)O[C@@H](C)c3c(N5CCOCC5)ccnc3N4)C2)CC1]

(*S), pure isomer, absolute stereochemistry undetermined

Compound 491 was synthesized following a similar sequence of reactions as for the synthesis of Compound 489 using Intermediate 706B instead of Intermediate 706A.

LC MS: confirms the MW (RT: 1.886, [M+H]⁺: 516, Method: 2)

¹H NMR (300 MHz, Chloroform-d) d (ppm) 1.46 (d, J=6.9 Hz, 3H), 1.83 (d, J=13.2 Hz, 4H), 2.69 (s, 1H), 2.91 (d, J=12.3 Hz, 3H), 3.03-3.27 (m, 3H), 3.58 (s, 2H), 3.72-4.07 (m, 8H), 4.43 (s, 1H), 5.68 (d, J=10.6 Hz, TH), 6.07 (q, J=6.9 Hz, 1H), 6.26 (d, J=16.8 Hz, 1H), 6.47-6.68 (m, 2H), 6.86 (s, 1H), 7.51 (s, 1H), 8.11 (d, J=5.4 Hz, 1H).

MP: 181.5° C. (Mettler Toledo MP50), uncorrected.

OR: −55.69° (589 nm, c 0.09333w/v, DMF, 23.0° C.).

### Compound 499 and Compound 498

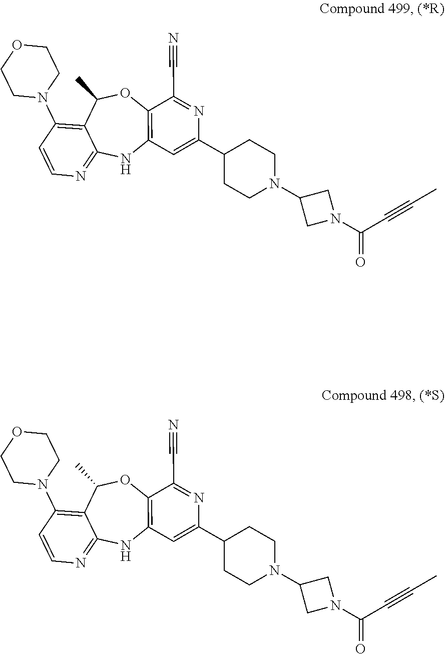 [SMILES: CC#CC(=O)N1CC(N2CCC(c3cc4c(c(C#N)n3)O[C@H](C)c3c(N5CCOCC5)ccnc3N4)CC2)C1 ; CC#CC(=O)N1CC(N2CCC(c3cc4c(c(C#N)n3)O[C@@H](C)c3c(N5CCOCC5)ccnc3N4)CC2)C1]

Both compounds are pure enantiomers but absolute stereochemistry is undetermined HBTU (443 mg, 1.168 mmol, 1.5 eq.) was added to a solution of Intermediate 138 (371 mg, 0.778 mmol), 2-butynoic acid (98 mg, 1.168 mmol, 1.5 eq.), and DIPEA (680 μL, 3.892 mmol, 5 eq.) in DCM (10 mL), and the mixture was stirred at room temperature for 2 h. The reaction was quenched by addition of saturated aqueous NaHCO₃ and the mixture was extracted with DCM. The organic layer was concentrated and the residue was purified by reverse phase chromatography (Phenomenex Gemini C18 30×100 mm 5 μm; gradient from 72% [25 mM NH₄HCO₃]−28% [ACN:MeOH (1:1)] to 36% [25 mM NH₄HCO₃]−64% [ACN:MeOH (1:1)]), followed by SFC (Column: LUX-CELLULOSE-1, isocratic 40% MeOH). The separated isomers were both purified again by reverse phase chromatography (Phenomenex Gemini C18 30×100 mm 5 μm; from 90% [65 mM NH₄OAc+ACN (90:10)]−10% [ACN:MeOH (1:1)] to 54% [65 mM NH₄OAc+ACN (90:10)]−46% [ACN:MeOH (1:1)]) to give Compound 499 (33 mg, yield: 19%) and Compound 498 (19 mg, 11%).

LCMS (Compound 499): confirms the MW (RT: 1.65, [M+H]⁺ 543, Method: 2).

MP (Compound 499): 156.4° C. (Mettler Toledo MP50), uncorrected.

OR (Compound 499): −8.2534° (589 nm, c 0.0973333 w/v, DMF, 23° C.).

¹H NMR (Compound 499): (300 MHz, Chloroform-d) d (ppm) 0.98 (s, 1H), 1.30 (d, J=27.7 Hz, 21H), 1.39 (d, J=6.9 Hz, 2H), 1.89 (d, J=7.0 Hz, 3H), 1.98 (s, 3H), 2.53 (s, 1H), 2.97 (d, J=12.7 Hz, 4H), 3.11 (d, J=12.6 Hz, 4H), 3.31 (s, 1H), 3.89 (d, J=5.0 Hz, 7H), 4.09 (s, 3H), 4.22 (s, 2H), 5.95 (d, J=7.3 Hz, 1H), 6.52 (s, 1H), 6.61 (s, 1H), 8.05 (s, 1H).

LCMS (Compound 498): confirms the MW (RT: 1.65, [M+H]⁺ 543, Method: 2).

MP (Compound 498): 198.2° C. (Mettler Toledo MP50), uncorrected.

OR (Compound 498): ±12.01610 (589 nm, c 0.0826667 w/v, DMF, 23° C.).

¹H NMR (Compound 498): (300 MHz, Chloroform-d) d (ppm) 0.93-1.11 (m, 1H), 1.25 (s, 2H), 1.39 (d, J=6.8 Hz, 2H), 1.89 (d, J=6.4 Hz, 31H), 1.97 (s, 3H), 2.53 (s, 1H), 2.98 (s, 5H), 3.10 (s, 4H), 3.89 (d, J=5.0 Hz, 7H), 4.11 (s, 3H), 4.23 (s, 2H), 5.95 (d, J=7.4 Hz, 1H), 6.61 (s, 2H), 8.04 (s, 1H).

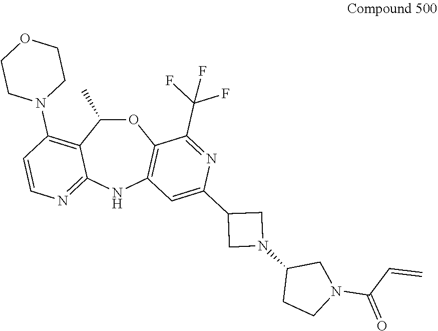 [SMILES: C=CC(=O)N1CC[C@H](N2CC(c3cc4c(c(C(F)(F)F)n3)O[C@@H](C)c3c(N5CCOCC5)ccnc3N4)C2)C1]

(*S, *S), pure isomer, absolute stereochemistry undetermined

Compound 500 was synthesized following a similar sequence of reactions as for the synthesis of Compound 504 using Intermediate 711B instead of Intermediate 711A.

LC MS: confirms the MW (RT: 2.056, [M+H]⁺: 545, Method: 2)

¹H NMR (300 MHz, Chloroform-d) d (ppm) 1.26 (s, 2H), 1.41 (d, J=6.9 Hz, 2H), 1.87 (d, J=131.5 Hz, 7H), 2.80-3.19 (m, 4H), 3.64 (s, 3H), 3.90 (d, J=4.8 Hz, 5H), 4.12 (s, 1H), 5.63-5.75 (m, 1H), 6.01 (d, J=7.1 Hz, 1H), 6.40 (d, J=3.7 Hz, 1H), 6.64 (d, J=5.4 Hz, 1H), 6.83 (d, J=10.6 Hz, 1H), 7.57 (s, 1H), 8.11 (d, J=5.3 Hz, 1H).

MP: 134.7° C. (Mettler Toledo MP50), uncorrected.

OR: −27° (589 nm, c 0.0687 w/v, DMF, 23.0° C.

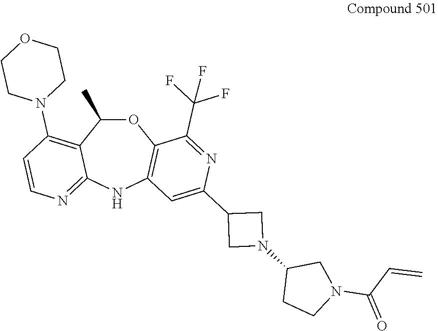 [SMILES: C=CC(=O)N1CC[C@H](N2CC(c3cc4c(c(C(F)(F)F)n3)O[C@H](C)c3c(N5CCOCC5)ccnc3N4)C2)C1]

(*R, *R), pure isomer, absolute stereochemistry undetermined

Compound 501 was synthesized following a similar sequence of reactions as for the synthesis of Compound 504 using Intermediate 711C instead of Intermediate 711A.

LC MS: confirms the MW (RT: 2.069, [M+H]⁺: 545, Method: 2)

¹H NMR (300 MHz, Chloroform-co d (ppm) 1.26 (s, 1H), 1.41 (d, J=6.8 Hz, 3H), 1.55-1.95 (m, 6H), 2.07 (s, 1H), 2.99 (dq, J=40.3, 8.2, 7.8 Hz, 4H), 3.65 (d, J=10.6 Hz, 4H), 3.91 (t, J=4.6 Hz, 4H), 4.07 (s, 1H), 5.69 (dd, J=9.3, 3.2 Hz, 1H), 6.01 (d, J 6.9 Hz, 1H), 6.27-6.47 (m, 1H), 6.64 (d, J=5.5 Hz, 1H), 6.85 (d, J=4.2 Hz, 1H), 7.56 (s, 1H), 8.11 (d, J=5.5 Hz, 1H).

MP: 144.7° C. (Mettler Toledo MP50), uncorrected.

OR: −45° (589 nm, c 0.1167 w/v, DMF, 23.0° C.).

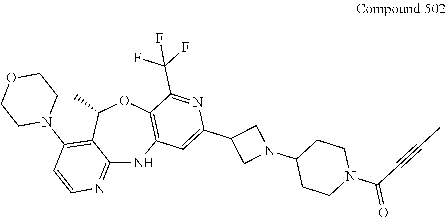 [SMILES: CC#CC(=O)N1CCC(N2CC(c3cc4c(c(C(F)(F)F)n3)O[C@@H](C)c3c(N5CCOCC5)ccnc3N4)C2)CC1]

(*S), pure isomer, absolute stereochemistry undetermined

Compound 502 was synthesized following a similar set of reactions as for the synthesis of Compound 505 starting from Intermediate 713B instead of Intermediate 713A.

### Data for Compound 502:

LC MS: confirms the MW (RT: 2.187, [M+H]⁺: 571, Method: 2)

¹H NMR (300 MHz, Chloroform-d) d (ppm) 8.10 (d, J=5.4 Hz, 1H), 7.91 (s, 1H), 6.90 (d, J=5.5 Hz, 1H), 6.62 (d, J=5.5 Hz, TH), 6.00 (q, J=7.0 Hz, 1H), 4.35-4.09 (m, 2H), 3.90 (t, J=4.6 Hz, 4H), 3.70 (s, 3H), 3.47-3.21 (m, 3H), 3.16-2.82 (m, 4H), 2.57-2.35 (m, 1H), 2.35-2.14 (m, 1H), 2.00 (s, 3H), 1.87-1.62 (m, 2H), 1.41 (d, J:=6.9 Hz, 3H), 1.36-1.16 (m, 2H).

MP: 154.7° C. (Mettler Toledo MP50), uncorrected.

OR: −27° (589 nm, c 0.168 w/v, DMF, 23.0° C.).

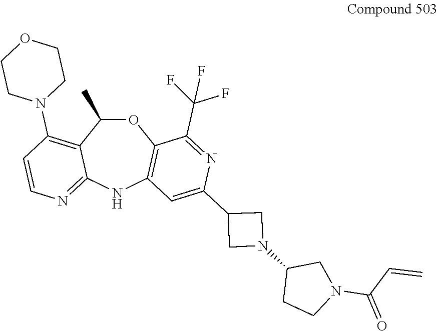 [SMILES: C=CC(=O)N1CC[C@H](N2CC(c3cc4c(c(C(F)(F)F)n3)O[C@H](C)c3c(N5CCOCC5)ccnc3N4)C2)C1]

(*R, *S), pure isomer, absolute stereochemistry undetermined

Compound 503 was synthesized following a similar sequence of reactions as for the synthesis of Compound 504 using Intermediate 711D instead of Intermediate 711A.

LC MS: confirms the MW (RT: 2.058, [M+H]⁺: 545, Method: 2)

¹H NMR (300 MHz, Chloroform-d) d (ppm) 1.28 (d, J=10.6 Hz, 2H), 1.41 (d, J=6.8 Hz, 3H), 1.87-2.10 (m, 3H), 2.93 (q, J=7.1, 5.7 Hz, 2H), 3.00-3.13 (m, 2H), 3.36 (s, 1H), 3.47-3.67 (m, 6H), 3.90 (d, J=4.6 Hz, 5H), 5.63-5.73 (m, 1H), 5.96-6.06 (m, 1H), 6.36-6.44 (m, 1H), 6.63 (d, J=5.4 Hz, 1H), 6.85 (d, J=12.8 Hz, 1H), 7.60 (s, 1H), 8.10 (d, J=5.5 Hz, 1H).

MP: 131.3° C. (Mettler Toledo MP50), uncorrected.

OR: +27° (589 nm, c 0.07 w/v, DMF. 23.0° C.).

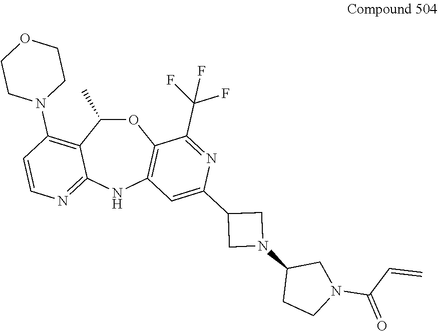 [SMILES: C=CC(=O)N1CC[C@@H](N2CC(c3cc4c(c(C(F)(F)F)n3)O[C@@H](C)c3c(N5CCOCC5)ccnc3N4)C2)C1]

(*S, *R), pure isomer, absolute stereochemistry undetermined

To a solution of Intermediate 712 (206 mg, 0.42 mmol) in DCM (5 mL), DIPEA (370 μL, 2.1 mmol) was added. The mixture was cooled in an ice bath, then acryloyl chloride [CAS: 814-68-6] (34 μL, 0.42 mmol) in DCM (340 μL) was added dropwise to the mixture and stirred at room temperature for 15 h. The reaction mixture was quenched by addition of saturated aqueous NaHCO₃ and extracted with DCM. The organic layer was separated, dried, filtered, and concentrated under vacuum. Purification by reverse phase chromatography (InterChim Uptisphere Strategy C18-HQ 5 μm 30×100 mm; gradient from 90% [0.1% HCOOH]−10% ACN to 54% [0.1% HCOOH]−46% ACN) afforded Compound 504 (148 mg, yield: 65%).

Chiral HPLC: confirms the MW (RT: 9.880 min, [M+H]⁺: 545.1, Method: 15); one isomer only.

LC MS: confirms the MW (RT: 2.065, [M+H]⁺: 545, Method: 2)

¹H NMR (300 MHz, Chloroform-d) d (ppm) 1.26 (s, 1H), 1.41 (d, J=6.7 Hz, 3H), 1.97 (d, J=26.1 Hz, 4H), 2.93 (q, J=7.0, 5.6 Hz, 2H), 2.99-3.11 (m, 2H), 3.33-3.44 (m, 1H), 3.57 (s, 4H), 3.86 (dt, J=31.4, 7.3 Hz, 7H), 5.68 (dd, J=9.4, 3.1 Hz, 1H), 6.00 (t, J=6.7 Hz, 1H), 641 (d, J=8.7 Hz, 1H), 6.63 (d, J=5.4 Hz, 1H), 6.86 (d, J=4.8 Hz, 1H), 7.58 (s, 1H), 8.10 (d, J=5.5 Hz, 1H)

MP: 133° C. (Mettler Toledo MP50), uncorrected.

OR: +44° (589 nm, c 0.076 w/v, DMF, 23.0° C.).

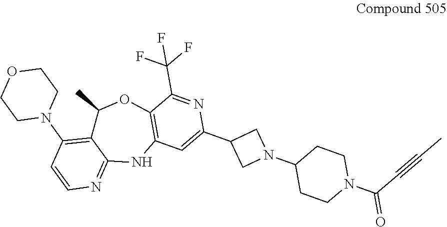 [SMILES: CC#CC(=O)N1CCC(N2CC(c3cc4c(c(C(F)(F)F)n3)O[C@H](C)c3c(N5CCOCC5)ccnc3N4)C2)CC1]

(*R), pure isomer, absolute stereochemistry undetermined

HBTU [CAS: 94790-37-1] (0.35 g, 0.92 mmol) was added to a solution of Intermediate 714 (0.31 g, 0.61 mmol), 2-butynoic acid [CAS: 590-93-2] (77 mg, 0.92 mmol) and DIPEA (0.53 mL, 3.06 mmol) in DCM (10 mL) at room temperature and the mixture was stirred for 2 h. The reaction mixture was poured into a saturated solution of NaHCO₃ and extracted with DCM. The combined organic layers were dried over MgSO₄, filtered, concentrated to dryness and purified by chromatography by reverse phase chromatography (InterChim Uptisphere Strategy C18-HQ 5 μm 30×100 mm PREP-LC Column; gradient from 90% [0.1% HCOOH]−10% [ACN:MeOH (1:1)] to 54% [0.1% HCOOH]−46% [ACN:MeOH (1:1)]) to afford Compound 505 (226 mg, yield: 65%).

LC MS: confirms the MW (RT: 2.295, [M+H]⁺: 571, Method: 2)

¹H NMR (300 MHz, Chloroform-d) d (ppm) 8.10 (d, J=5.4 Hz, 1H), 8.05 (s, 1H), 6.91 (d, J=5.4 Hz, 1H), 6.62 (d, J=5.5 Hz, 1H), 6.00 (q, J=7.0 Hz, 1H), 4.31-4.06 (m, 2H), 3.90 (s, 4H), 3.69 (s, 3H), 3.46-3.21 (m, 3H), 3.14-2.82 (m, 4H), 2.48-2.36 (m, 1H), 2.35-2.17 (m, 1H), 1.99 (s, 3H), 1.87-1.65 (in 2H), 1.41 (d, J=6.9 Hz, 3H), 1.36-1.11 (m, 2H).

MP: 153° C. (Mettler Toledo MP50), uncorrected.

OR: +28° (589 nm, c 0.252 w/v, DMF, 23.0° C.).

### Compound 517 and Compound 518

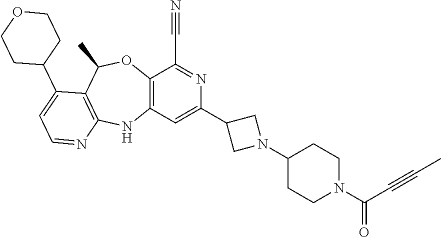 [SMILES: CC#CC(=O)N1CCC(N2CC(c3cc4c(c(C#N)n3)O[C@H](C)c3c(C5CCOCC5)ccnc3N4)C2)CC1] [2029] Compound 517 (*R) pure isomer, absolute stereochemistry undetermined.

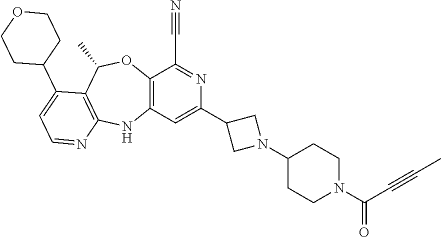 [SMILES: CC#CC(=O)N1CCC(N2CC(c3cc4c(c(C#N)n3)O[C@@H](C)c3c(C5CCOCC5)ccnc3N4)C2)CC1]

Compound 518 (*S), pure isomer, absolute stereochemistry undetermined

HBTU [CAS: 94790-37-1] (816 mg, 2.15 mmol) was added to a solution of Intermediate 720 (0.66 g, 1.44 mmol), 2-butynoic acid [CAS: 590-93-2] (181 mg, 2.15 mmol) and DIPEA (1.25 mL, 7.18 mmol) in DCM (10 mL) at room temperature and the mixture was stirred for 3 h. The reaction mixture was poured into a saturated solution of NaHCO₃ and extracted with DCM. The combined organic layers were washed with a saturated solution of NaHCO₃ and brine, dried over MgSO₄, filtered, concentrated to dryness. Purification by silica gel chromatography (eluent MeOH in DCM) followed by chiral SFC (Stationary phase: CHIRALPAK AD-H 5 μm 250*30 mm, Mobile phase: 55% CO₂, 45% mixture of EtOH/iPrOH 50/50 v/v (+0.6% Et₃N)) afforded Compound 517 (161 mg, yield: 20%) and Compound 518 (166 mg, yield: 20 Data for Compound 517:

LC MS: confirms the MW (RT: 2.41, [M+H]⁺: 526, Method: 1)

¹H NMR (500 MHz, DMSO-d₆, 28° C.): δ (ppm) 10.25 (s, 1H), 8.19 (d, J=5.0 Hz, 1H), 7.44 (s, 1H), 6.96 (d, J=5.0 Hz, 1H), 6.15 (q, J=6.9 Hz, 1H), 3.84-4.01 (m, 4H), 3.51-3.64 (m, 5H), 3.34-3.44 (m, 1H), 3.20 (br d, J=4.7 Hz, 2H), 3.09 (ddd, J=12.9, 9.5, 3.5 Hz, 1H), 2.33-2.47 (m, 1H), 2.01 (s, 3H), 1.59-1.78 (m, 5H), 1.55 (br d, J=12.3 Hz, 1H), 1.32 (d, J=6.9 Hz, 3H), 1.03-1.25 ppm (m, 3H)OR: −22.19° (589 nm, c 0.32 w/v, DMF, 20.0° C.).

OR: −22.19° (589 nm, c 0.32 w/v, DMF, 20.0° C.).

SFC: RT: 1.47, 100%, [M+H]⁺ 527, Method: 5

### Data for Compound 518:

LC MS: confirms the MW (RT: 2.41, [M+H]⁺: 526, Method: 1)

¹H NMR (500 MHz, DMSO-d₆, 28° C.): a (ppm) 10.25 (s, 1H), 8.19 (d, J=5.0 Hz, 1H), 7.44 (s, 1H), 6.96 (d, J=5.4 Hz, 1H), 6.15 (q, J=6.9 Hz, 1H), 3.84-4.01 (m, 4H), 3.51-3.64 (m, 5H), 3.33-3.44 (m, 1H), 3.15-3.23 (m, 2H), 3.09 (ddd, J=12.8, 9.5, 3.2 Hz, 1H), 2.32-2.39 (m, 1H), 2.01 (s, 3H), 1.53-1.75 (m, 6H), 1.32 (d, J=6.9 Hz, 3H), 1.03-1.24 ppm (m, 3H)

OR: +17.31° (589 nm, c 0.26 w/v, DMF, 20.0° C.).

SFC: RT: 1.97, 98%, [M+H] 527, Method: 5

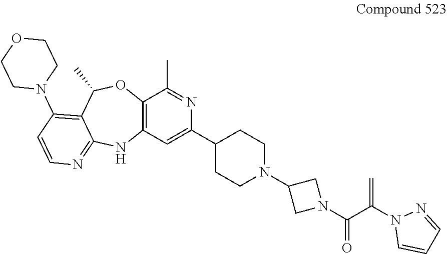 [SMILES: C=C(C(=O)N1CC(N2CCC(c3cc4c(c(C)n3)O[C@@H](C)c3c(N5CCOCC5)ccnc3N4)CC2)C1)n1cccn1]

(*S), pure enantiomer but absolute stereochemistry undetermined

HBTU (227 mg, 0.599 mmol, 1.5 eq.) was added to a solution of Intermediate 77 (180 mg, 0.399 mmol), Intermediate 140 (72 mg, 0.519 mmol, 1.3 eq.), and DIPEA (348 μL, 1.995 mmol, 5 eq.) in DCM (5 mL), and the mixture was stirred at room temperature for 3 h. The mixture was poured into saturated aqueous NaHCO₃ and the mixture was extracted with DCM. The organic layer was washed with aqueous NaHCO₃, brine and concentrated. The residue was purified by column flash chromatography (SiO₂, DCM to DCM/MeOH) followed by reverse phase chromatography to give Compound 523 (36 mg, yield: 15%).

LCMS: confirms the MW (RT: 1.58, [M+H]⁺ 571, Method: 2).

MP: 148.0° C. (Mettler Toledo MP50), uncorrected.

OR: −52.58331° (589 nm, c 0.080000 w/v, DMF, 23° C.).

¹H NMR (300 MHz, Chloroform-d) d (ppm) 1.35 (d, J=6.9 Hz, 3H), 1.73 (d, J=11.0 Hz, 1H), 1.94 (q, J=11.0, 10.5 Hz, 5H), 2.43 (s, 3H), 2.50-2.66 (m, 1H), 2.81 (d, J=10.5 Hz, 1H), 2.92 (td, J=12.3, 11.4, 5.4 Hz, 3H), 3.10 (ddd, J=21.2, 11.0, 5.4 Hz, 311), 3.89 (td, J=7.7, 6.6, 3.8 Hz, 611), 4.03 (dd, J=10.6, 5.5 Hz, 1H), 4.20 (dd, J=10.4, 7.4 Hz, 1H), 5.47 (s, 1H), 5.85 (s, 1H), 5.95 (q, J=6.9 Hz, 1H), 6.38 (d, J=2.6 Hz, 2H), 6.56 (d, J=5.5 Hz, 1H), 7.11 (s, 1H), 7.66 (d, J=1.7 Hz, 1H), 7.76 (d, J=2.5 Hz, 1H), 8.06 (d, J=5.5 Hz, 1H).

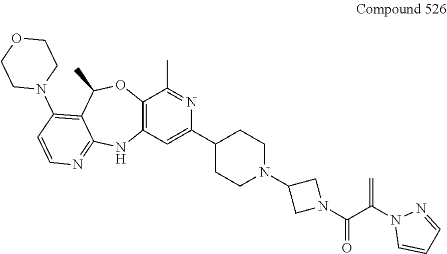 [SMILES: C=C(C(=O)N1CC(N2CCC(c3cc4c(c(C)n3)O[C@H](C)c3c(N5CCOCC5)ccnc3N4)CC2)C1)n1cccn1]

(*R), pure enantiomer but absolute stereochemistry undetermined

Compound 526 was prepared using a procedure analogous to Compound 523, starting from Intermediate 76 instead of Intermediate 77.

LCMS: confirms the MW (RT: 1.59, [M+H]⁺ 571, Method: 2).

MP: 131.3° C. (Mettler Toledo MP50), uncorrected.

OR: +36.4615° (589 nm, c 0.0866667 w/v, DMF, 23° C.).

¹H NMR (300 MHz, Chloroform-d) d (ppm) 1.35 (d, J=6.9 Hz, 3H), 1.71 (t, J=11.0 Hz, 1H), 1.94 (q, J=11.0, 10.5 Hz, 5H), 2.43 (s, 3H), 2.58 (q, J=11.4, 10.4 Hz, 1H), 2.81 (d, J=10.4 Hz, 1H), 2.92 (dq, J=11.7, 5.4, 4.5 Hz, 3H), 3.10 (ddd, J=21.2, 11.1, 5.4 Hz, 3H), 3.89 (td, J=7.4, 6.5, 3.8 Hz, 6H), 4.03 (dd, J=10.6, 5.5 Hz, 1H), 4.20 (dd, J=10.4, 7.4 Hz, 1H), 5.47 (s, 1H), 5.85 (s, 1H), 5.95 (q, J=6.9 Hz, 1H), 6.38 (d, J=2.6 Hz, 2H), 6.56 (d, J=5.5 Hz, 1H), 7.10 (s, 1H), 7.66 (d, J=1.7 Hz, 1H), 7.76 (d, J=2.4 Hz, 1H), 8.06 (d J=5.5 Hz, 1H).

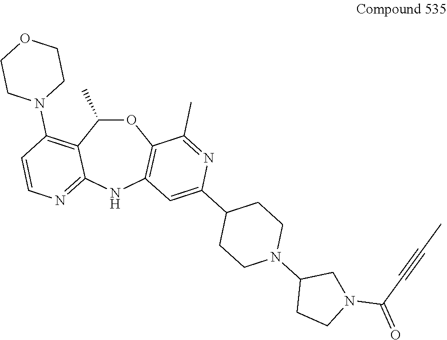 [SMILES: CC#CC(=O)N1CCC(N2CCC(c3cc4c(c(C)n3)O[C@@H](C)c3c(N5CCOCC5)ccnc3N4)CC2)C1]

(*S), absolute stereochemistry undetermined, mixture of diastereoisomers

A mixture of Intermediate 724 (0.6 g, 1.517 mmol), 3-Pyrrolidinone, 1-(1-oxo-2-butyn-1-yl) [CAS: 2152134-73-9] (460 mg, 3.043 mmol) NaBH(OAc)₃ [CAS: 56553-60-7](0.965 g, 4.551 mmol) and AcOH (0.26 mL) in DCE (12 mL) was stirred at room temperature for 18 h. The reaction mixture was poured onto a 10% aqueous solution of K₂CO₃ and DCM and the aqueous layer was extracted with DCM. The combined organic layer was dried over MgSO₄, filtered, and the solvent was evaporated to dryness. Purification via column chromatography (Stationary phase: irregular silica 40 g, Gradient from 0.1% NH₄OH, 97% DCM, 3% DCM to 0.1% NH₄OH, 95% DCM, 5% DCM) followed by SFC purification (Stationary phase: 2 Ethylpyridine 6 μm 250×21.2 mm, Mobile phase: 85% CO₂, 15% MeOH) and crystallization from ACN afforded Compound 535 (75 mg, yield: 9%).

LC MS: confirms the MW (RT: 2.30, [M+H]⁺: 530, Method: 1)

¹H NMR (400 MHz, DMSO-d₆, 23° C.): δ (ppm) 9.57 (d, J=4.6 Hz, 1H), 8.09 (d, J=5.1 Hz, 1H), 6.88 (s, 1H), 6.69 (d, J=5.4 Hz, 1H), 5.91 (q, J=6.6 Hz, 1H), 3.85-3.94 (m, 0.5 H (diastereoisomer A)), 3.71-3.84 (m, 5H), 3.64 (dd, J=11.9, 7.0 Hz, 0.5 H (diastereoisomer B)), 3.43-3.53 (m, 1H), 3.17-3.30 (m, 1H), 2.96-3.06 (m, 2H), 2.76-2.94 (m, 6H), 2.36-2.45 (m, 1H), 1.98-2.19 (m, 6H), 1.54-1.88 (m, 5H), 1.24 ppm (d, J=6.8 Hz, 3H)

OR: +33.64° (589 nm, c 0.22 w/v, DMF, 20.0° C.).

### Example B: Analytical Characterization of Compounds

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g., scanning range, dwell time . . . ) in order to obtain ions allowing the identification of the compound)s nominal monoisotopic molecular weight (MW). Data acquisition was performed with appropriate software. Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . ). For molecules with multiple isotopic patterns (Br, Cl), the reported value is the one obtained for the lowest isotope mass. All results were obtained with experimental uncertainties that are commonly associated with the method used. Hereinafter. “SQD” means Single Quadrupole Detector, “MSD” lass Selective Detector, “RT” room temperature, “BFIT” bridged ethylsiloxane/silica hybrid, “DAD” Diode Array Detector, “HSS” High Strength silica.

LCMS Method Codes (Flow Expressed in mL/Min, Column Temperature (T) in ° C., Run Time in Minutes)

| Method | | | | | Flow | Run |
|---|---|---|---|---|---|---|
| Code | Instrument | column | mobile phase | gradient | Col T | time |
| 1 | Waters: | Waters | A: CH₃COONH₄ | 84.2% A/15.8% | 0.343 | 6.07 |
| | Acquity ® | BEH ® | 7 mM 95%/ | B for 0.49 min, | 40 | |
| | UPLC - | C18 (1.7 | CH₃CN 5% | to 10.5% A in | | |
| | DAD and | μm, 2.1 × | B: CH₃CN | 2.18 min, held | | |
| | Quattro ™ | 100 mm) | | for 1.94 min, | | |
| | | | | back to 84.2% | | |
| | | | | A/15.8% B in | | |
| | | | | 0.73 min, held | | |
| | | | | for 0.73 min. | | |
| 2 | Agilent: | YMC: Pack | A: HCOOH | 95% A to 5% | 2.6 s | 6 |
| | 1100- | ODS-AQ (3 | 0.1% in water, | A in 4.8 min, | 35 | |
| | DAD and | μm, 4.6 × | B: CH3CN | held for 1 min, | | |
| | MSD | 50 mm) | | back to 95% | | |
| | | | | A in 0.2 min. | | |
| 3 | Agilent | YMC-pack | A: 0.1% | From 95% A | 2.6 | 6.8 |
| | 1260 | ODS-AQ | HCOOH in | to 5% A in 4.8 | 35 | |
| | Infinity | C18 (50 × | H₂O | min, held for | | |
| | DAD | 4.6 mm, 3 μm) | B: CH3CN | 1.0 min, to 95 | | |
| | TOF-LC/MS | | | % A in 0.2 min. | | |
| | G6224A | | | | | |
| 4 | Waters: | Waters: | A: 95% | From 95% A to | 1 | 5 |
| | Acquity ® | BEH | CH₃COONH₄ | 5% A in 4.6 | 50 | |
| | IClass | C18 (1.7 μm, | 6.5 mM + | min, held for | | |
| | UPLC ® - | 2.1 × 50 mm) | 5% CH₃CN | 0.4 min | | |
| | DAD and | | B: CH₃CN | | | |
| | Xevo | | | | | |
| | G2-S | | | | | |
| | QTOF | | | | | |
| 5 | Waters: | Waters: | A: 95% | From 95% A to | 1 | 5 |
| | Acquity ® | BEH | CH₃COONH₄ | 5% A in 4.6 | 50 | |
| | UPLC ® - | C18 (1.7 μm, | 6.5 mM + | min, held for | | |
| | DAD and | 2.1 × 50 mm) | 5% CH₃CN | 0.4 min | | |
| | SQD | | B: CH₃CN | | | |
| 8 | Waters: | Waters: | A: 95% | From 95% A | 1 | 5 |
| | Acquity ® | BEH | CH₃COONH₄ | to 5% A in | 50 | |
| | IClass | C18 (1.7 μm, | 6.5 mM + | 4.6 min, held | | |
| | UPLC ® - | 2.1 × 50 mm) | 5% CH₃CN | for 0.4 min | | |
| | DAD and | | B: CH₃CN | | | |
| | Xevo | | | | | |
| | G2-S | | | | | |
| | QTOF | | | | | |
| 9 | Agilent | Phenomenex | A: 50 mM | From 95% A | 0.7 | 6.2 |
| | 1100 | Kinetex | NH₄OAc in | to 5% A in 4.8 | 35 | |
| | HPLC | C18 (50 × | H₂O | min, held for | | |
| | DAD | 2.1 mm, 2.6 μm) | B: CH₃CN | 1.0 min, to 95% | | |
| | LC/MS | | | A in 0.2 min. | | |
| | G1956A | | | | | |
| 10 | Waters: | Waters | A: | From 84.2% | 0.343 | 6.1 |
| | Acquity ® | BEH ® | CH₃COONH₄ | A/15.8% B to | 40 | |
| | H-Class - | C18 (1.7 μm, | 7 mM 95%/ | 10.5% A in | | |
| | DAD and | 2.1 × 100 mm) | CH₃CN 5% | 2.18 min, held | | |
| | SQD2 ™ | | B: CH₃CN | for 1.96 min, | | |
| | | | | back to 84.2% | | |
| | | | | A/15.8% B in | | |
| | | | | 0.73 min, held | | |
| | | | | for 0.49 min. | | |
| 11 | Waters: | Waters | A: | From 95% | 0.5 | 3.5 |
| | Acquity | BEH ® | CH₃COONH₄ | A/5% B to 5 | 40 | |
| | H-Class - | C18 (1.7 μm, | 7 mM 95%/ | % A in 1 min, | | |
| | DAD and | 2.1 × 50 mm) | CH₃CN 5% | held for | | |
| | SQD2 ™ | | B: CH₃CN | 1.6 min, back | | |
| | | | | to 95% A/5% | | |
| | | | | B in 0.2 min, | | |
| | | | | held for 0.5 | | |
| | | | | min. | | |
| 12 | Agilent | Waters | A: water with | 90% A held | 0.8 | 10 |
| | 1200 | Xbridge- | 0.04% TFA | for 0.8 min. | 50 | |
| | equiped | C18 | B: CH₃CN | Then to 20% | | |
| | with | column (5 μm, | with 0.02% | A and 80% B | | |
| | MSD | 2.0 μ 50 mm) | TFA | in 3.7 minutes, | | |
| | 6110 or | | | held for 3 min. | | |
| | equivalent | | | Return to 90% | | |
| | | | | A in 2 min and | | |
| | | | | hold for 0.5 | | |
| | | | | min. | | |
| 13 | Waters: | Agilent: | A: 95% | From 95% A | 0.8 | 5 |
| | Acquity ® | RRHD C18 | CH₃COONH₄ | to 5% A in | 50 | |
| | IClass | (1.8 μm, | 6.5 mM + 5% | 4.5 min, | | |
| | UPLC ® - | 2.1 × 50 mm) | CH₃CN | held for 0.5 | | |
| | DAD and | | B: CH₃CN | min | | |
| | SQD | | | | | |
| 14 | Agilent | YMC pack | A: Water + 0.1 | From 100% A | 2.6 | 5.0 |
| | 1100 | ODS-AQ | % Formic acid | held for 0.2 | 35 | |
| | HPLC | C18 | B: CH₃CN | min | | |
| | DAD | column (50 | | To 50% A/50% | | |
| | LC/MS | mm long × | | B in 4.5 min | | |
| | G1956A | 4.6 mm I.D..; | | and to 5% | | |
| | | 3 μm particle | | A/95% B in | | |
| | | size) | | 0.1 min | | |
| | | | | held for 1.0 | | |
| | | | | min | | |
| | | | | to 95% A in | | |
| | | | | 0.2 min | | |
| 15 | Agilent | Phenomen | A: 0.1% | Held 95% A | 1.0 | 30 |
| | 1260 | ex-Lux | Diethylamine | for 0.5 min | 25 | |
| | Infinity | Amylose-1 | in heptane | From 95% A | | |
| | DAD | (150 × 4.6 | B: 0.1% | to 0% A in | | |
| | TOF- | mm, 5 μm) | Diethylamine | 15.1 min | | |
| | LC/MS | | in iPrOH | held for 3.0 | | |
| | G6224A | | | min | | |
| | | | | to 95% A in | | |
| | | | | 0.4 min. | | |

### LCMS Results (RT Means Retention Time)

| Compound | |
|---|---|
| number | LCMS results |
| 3 | confirms the MW (RT: 2.57, [M + H]+ 475, Method: 1) |
| 4 | confirms the MW (RT: 2.56, [M + H]+ 476, Method: 1) |
| 20 | confirms the MW (RT: 1.52, [M + H]+ 490, Method: 2) |
| 83 | confirms the MW (RT: 1.37, [M + H]+ 477, Method: 3) |
| 164 | confirms the MW (RT: 1.47, [M + H]+ 486, Method: 2) |
| 263 | confirms the MW (RT: 1.42 [M + H]+ 491, Method: 2) |
| 172 | confirms the MW (RT: 1.44 [M + H]+ 491, Method: 2) |
| 173 | confirms the MW (RT: 1.46 [M + H]+ 491, Method: 2) |
| 262 | confirms the MW (RT: 1.34 [M + H]+ 505, Method: 2) |
| 265 | confirms the MW (RT: 1.28 [M + H]+ 505, Method: 2) |
| 272 | confirms the MW (RT: 1.45 [M + H]+ 517, Method: 2) |
| 279 | confirms the MW (RT: 1.34 [M + H]+ 503, Method: 2) |
| 306 | confirms the MW (RT: 1.63 [M + H]+ 502, Method: 2) |
| 344 | confirms the MW (RT: 2.28, [M + H]+ 517, Method: 1) |
| 345 | confirms the MW (RT: 2.27, [M + H]+ 517, Method: 1) |
| 351 | confirms the MW (RT: 1.73 [M + H]+ 515, Method: 2) |
| 369 | confirms the MW (RT: 1.28 [M + H]+ 555, Method: 2) |
| 378 | confirms the MW (RT: 1.24 [M + H]+ 543, Method: 2) |
| 383 | confirms the MW (RT: 1.41, [M + H]+ 553, Method: 3) |
| 403 | confirms the MW (RT: 1.57, [M + H]+ 529, Method: 2) |
| 481 | confirms the MW (RT: 2.45, [M + H]+ 528, Method: 1) |
| 485 | confirms the MW (RT: 2.45, [M + H]+ 528, Method: 1) |
| 499 | confirms the MW (RT: 1.65, [M + H]+ 543, Method: 2) |
| 498 | confirms the MW (RT: 1.65, [M + H]+ 543, Method: 2) |
| 523 | confirms the MW (RT: 1.58, [M + H]+ 571, Method: 2) |
| 526 | confirms the MW (RT: 1.59, [M + H]+ 571, Method: 2) |

### SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Analytical SFC-MS Methods (Flow expressed in mL/min; column temperature (Col T) in ° C.; Run lime in Minutes, Backpressure (BPR) in bars unless otherwise indicated.

“iPrNH₂” means isopropylamine, “iPrOH” means 2-propanol, “EtOH” means ethanol, “min” mean minutes, “DEA” means diethylamine.

### SFC Methods:

| Method | | mobile | | Flow | Run time | |
|---|---|---|---|---|---|---|
| code | column | phase | gradient | Col T | BPR | |
| 1 | Chiralcel | CO₂ 80% + | isocratic | 3.5 | 3 | min |
| | OJ-3 | (EtOH | | 35 | 1500 | PSI |
| | 3 μm | 20% + 0.3% | | | | |
| | | iPrNH₂) | | | | |
| 2 | Chiralpak^{R} | A: CO₂ | 30% B hold | 3.5 | 3 | min |
| | AS-3 | B: MeOH | 3 min, | 35 | 1500 | PSI |
| | column | (0.3% | | | | |
| | (3 μm, | iPrNH₂) | | | | |
| | 100 × | | | | | |
| | 4.6 mm) | | | | | |
| 3 | Whelk- | CO₂ 45% + | isocratic | 3.5 | 6 | min |
| | O1(S, S) | (MeOH | | 35 | 1500 | PSI |
| | 3 μm | 55% + 0.3% | | | | |
| | | iPrNH₂) | | | | |
| 4 | Chiralcel | CO₂ 50% + | isocratic | 3.5 | 3 | min |
| | OD-3 | (EtOH | | 35 | 1500 | PSI |
| | 3 μm | 50% + 0.3% | | | | |
| | | iPrNH₂) | | | | |
| 5 | Chiralpak | CO₂ 60% + | isocratic | 3.5 | 6 | min |
| | AD-3 | (EtOH + | | 35 | 1500 | PSI |
| | 3 μm | iPrOH | | | | |
| | | 50/50) | | | | |
| | | 40% + 0.3% | | | | |
| | | iPrNH₂) | | | | |
| 6 | Chiralpak | CO₂ 40% + | isocratic | 3.5 | 6 | min |
| | AD-3 | (iPrOH | | 35 | 1500 | PSI |
| | 3 μm | 60% + 0.3% | | | | |
| | | iPrNH₂) | | | | |
| 7 | Chiralpak | CO₂ 75% + | isocratic | 3.5 | 6 | min |
| | AS-3 | (MeOH | | 35 | 1500 | PSI |
| | 3 μm | 25% + 0.3% | | | | |
| | | iPrNH₂) | | | | |
| 8 | Whelk- | CO₂ 55% + | isocratic | 3.5 | 6 | min |
| | O1(S, S) | (MeOH | | 35 | 1500 | PSI |
| | 3 μm | 45% + 0.3% | | | | |
| | | iPrNH₂) | | | | |

TABLE
Analytical SFC data - Rₜ means retention time (in minutes),
[M + H]⁺ means the protonated mass of the compound,
method refers to the method used for (SFC)MS analysis of
enantiomerically pure compounds. “No.” means number.

| Compound | | | |
|---|---|---|---|
| No. | SFC Method | Rt | [M + H]⁺ |
| 344 | 1 | 1.11 | 517 |
| 345 | 1 | 1.50 | 517 |
| 481 | 2 | 2.27 | 528 |
| 485 | 2 | 1.61 | 528 |

### NMR

¹H NMR spectra were recorded on Bruker Avance III 400 MHz and Avance NEO 400 MHz spectrometers. CDCl₃ was used as solvent, unless otherwise mentioned. The chemical shifts are expressed in ppm relative to tetramethylsilane.

### Example C: Pharmacological Assays

### Expression and Purification of a Trimeric Complex of CDK7, Cyclin H, and MAT1:

Human CDK7 (amino acids 1-346) containing an N-terminal His₆-tag followed by a tobacco etch virus (TEV) protease cleavage site, human MAT1 (amino acids 1-309) and human cyclin H (amino acids 1-323) were co-expressed in the baculovirus-SF9 insect cell expression system to generate a trimeric complex. Cell pellets were collected 72 h post-infection and were resuspended by Dounce homogenization in 20 mM Hepes-NaOH (pH 8.0), 300 mM NaCl, 10% glycerol, 2 mM dithiothreitol DTT), and 20 mM Imidazole supplemented with cOmplete™ Protease Inhibitor Cocktail (Roche) and 25 U/mL Benzonase® Nuclease HC according to the manufacturer's instructions. Cells were lysed by passing through a Microfluidics M110Y Microfluidizer 3 times at 600 kPa followed by centrifugation at 38,000×g at 4° C. for 1 hour. The supernatant was loaded onto a pre-equilibrated HisTrap HP column and eluted in 20 mM Hepes-NaOH (pH 8.0), 50 mM NaCl, 10% glycerol, 2 mM DTT, and 400 mM Imidazole. The eluate was further purified by gel filtration on a Superdex S200 16/60 column and eluted with 20 mM Hepes-NaOH (pH 7.5), 50 mM NaCl, 10% Glycerol, 2 mM DTT. Fractions containing a trimeric complex of CDK7, cyclin H, and MAT1 in a 1:1:1 ratio were pooled and concentrated to 3 mg/mL in a 10 kDa MWCO concentrator, and diluted to a final concentration of 1.6 mg/mL in 11.1 mM Hepes-NaOH (pH 8.0), 27.8 mM NaCl, 1.1 mM DTT and 50% glycerol.

### Materials

ATP, phosphoenolpyruvate (PEP), NADH, MgCl₂, Triton X-100 (10% solution), pyruvate, kinase/lactate dehydrogenase, 384-well assay plates (Greiner UV-Star Clear), and 384-well compound dilution plates (Greiner bio-one) were purchased from Sigma-Aldrich (St. Louis, MO). 1M Tris-HCl (pH 7.4) and CDK7/9 tide were obtained from Teknova (Hillister, CA) and Anaspec (Freemont, CA), respectively.

### In Vitro CDK7 Assay and Determination of Potency for Irreversible Covalent Inhibitors:

CDK7 activity is measured by following the production of ADP generated from ATP-dependent phosphorylation of the peptide substrate derived from RNA Pol II (CDK7/9 tide) by CDK7. Pyruvate kinase converts ADP and phosphoenolpyruvate (PEP) to ATP and pyruvate. Lactase dehydrogenase catalyzes pyruvate to lactate with a concomitant conversion of NADH to oxidized form NAD⁺, which is spectrophotometrically measured at 340 nm. The CDK7 assay was performed in 384-well microplates with a final volume of 100 □L. Inhibitor serial dilutions and liquid handing for the assay were performed by using Janus from PerkinElmer (Downers Grove, IL) and Tempest from Formulatrix (Bedford, MA), respectively. To determine inhibitor potency of irreversible covalent inhibitors (k_{inact}/K_{I} ratios), 500 nL of inhibitor in DMSO (or DMSO for controls) was added to the assay plate using Echo 555 from Labcyte (San Jose, CA) followed by 50 □L of assay mixture consisting of 600 □M peptide substrate (CDK7/9 tide, YSPTSPSYSPTSPSYSPTSPSKKKK), 1 mM ATP, 1 mM PEP, 200 □M NADH, 1.2-2 units of PK, 1.8-2.8 units of LDH, 20 mM Tris-HCl (pH 7.4), 10 mM MgCl₂, and 0.004% Triton X-100. Reactions were initiated by the addition of 50 □L of 40 nM CDK7/cyclinH/MAT1 trimeric complex in 20 mM Tri-HCl (pH 7.4), 10 mM MgCl₂, and 0.004% Triton X-100. The assay plates were centrifuged at 3220 g for 5 min using Centrifuge 5810 from Eppendorf (Hauppauge, NY) and then the absorbance changes were read at 340 nm at room temperature using Infinite M1000 from Tecan (Männedorf, Switzerland) every 2 min for 8 hours. For data analysis to determine potency (k_{inact}/K_{I} ratios), the reaction progress curves corresponding to the linear range of the DMSO control were fit to equation 1, where Vₒ is the initial rate in Abs/sec and t is time in seconds, yielding the first order rate constant for enzyme inactivation (k_{obs}) at each inhibitor concentration. The k_{obs} values were then plotted versus inhibitor concentration ([I]) and fit to equation 2 where k_{inact} is the maximal rate of inactivation that is achieved at infinite concentration of inhibitor and K_{I} is the inhibitor concentration that yields half the rate of maximal inactivation. When [I]<<K_{I}, equation 2 is simplified to equation 3. Thus, at inhibitor concentrations of well below K_{I}, a plot of k_{obs} versus inhibitor concentration ([I]) is linear, and the slope of the line is equal to k_{inact}/K_{I}.

$$Product = \frac{{V}_{0}}{{k}_{obs}} [ 1 - exp ( - {k}_{obs} t ) ] \qquad ( 1 )$$

$${k}_{obs} = \frac{{k}_{inact} [ I ]}{{K}_{I} + [ I ]} \qquad ( 2 )$$

$${k}_{obs} = \frac{{k}_{inact}}{{K}_{I}} [ I ] \qquad ( 3 )$$

### Imaging-Based Cellular RNA PolII Ser5 Phosphorylation Assay:

To evaluate inhibition of CDK7 kinase activity, a 384-well automated imaging assay was used. This assay detects Serine 5 phosphorylation on a unique heptapeptide sequence in the C-terminal domain of Rpb1 subunit of RNA polymerase II, the downstream substrate of CDK7. This heptapeptide sequence is repeated up to 52 times in the CTD of Rpb1.

### Materials

A549 adenocarcinoma human alveolar basal epithelial cells (ATCC, CCL-185), rabbit Phospho-Rpb1 CTD (Ser5) antibody (D9N51 (Cell Signaling Technology)), DMEM (Sigma), Fetal Bovine Serum (Biowest), L-glutamine (Sigma), Penicillin/Streptomycin (Life Technologies), Sodium Pyruvate (Sigma), Hepes (Sigma), poly-D-lysine coated μclear 384 black plates (Greiner), formaldehyde (PolySciences), D-PBS (Sigma), Methanol (Sigma), Alexa Fluor 488 goat anti rabbit IgG secondary antibody (Life Technologies), HCS CellMask™ Deep Red stain (Life Technologies), Hoechst 33258 (Invitrogen).

RNA Polymerase II Serine 5 phosphorylation was detected using a specific rabbit Phospho-Rpb1 CTD (Ser5) antibody. A549 adenocarcinoma human alveolar basal epithelial cells were seeded in 20 μl medium (DMEM supplemented with 1% Fetal Bovine Serum (heat inactivated 30′ 56° C.), 2 mM L-glutamine, 50 U/ml penicillin 50 μg/ml streptomycin, 1 mM sodium pyruvate and 50 mM hepes) at 1000 cells/well and cultured in poly-D-lysine coated μclear 384 black plates for 20 hours at 37° C. and 5% CO₂.

After incubation cells were challenged with compound for 3 hours at 37° C. and 5% CO₂. DMSO was used as high control and as low control 10 μM of the following reference compound was used:

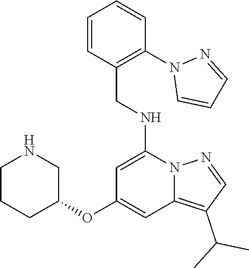 [SMILES: CC(C)c1cnn2c(NCc3ccccc3-n3cccn3)cc(O[C@@H]3CCCNC3)cc12]

40 nl of test compounds and controls were spotted in cell plates using Echo Liquid Handler (Echo 550, Labcyte). Incubation was followed by 20 minutes fixation with 20 μl 10% formaldehyde at room temperature. Medium/formaldehyde solution was removed, plates were washed 3 times with 30 μl D-PBS (w/o Ca²⁺ and Ma²⁺) and permeabilization was done by adding 20 μl ice cold methanol for 20 minutes. Cells were washed again 3 times with 30 μl D-PBS and 20 μl blocking buffer (25 ml fetal bovine serum in 500 ml D-PBS) was added for 1 hour.

After removing blocking buffer 20 μl 1/1000 primary antibody rabbit Phospho-Rpb1 CTD (Ser5) antibody was added which binds to the phosphorylated Serine5 of the heptapeptide sequences in the CTD of Rpb1. Primary antibody was removed, and plates were washed 3 times with 30 μl D-PBS followed by addition of 20 μl 1/2000 Alexa Fluor 488 goat anti rabbit IgG secondary antibody for final detection of Phospho-Rpb1 CTD (Ser5) together with 1/5000 HCS CellMask™ Deep Red stain for membrane staining and 1/5000 Hoechst 33258 for nucleus staining. Last, plates were washed 2 times with 30 μl D-PBS and wells were filled with 40 μL D-PBS, plates were sealed (Thermowell sealing tape) and stored at 4° C. until reading. Plates were read with Opera Phenix (Perkin Elmer) with 10× air objective. Data were calculated and analyzed in Phaedra.

IC₅₀ values were calculated using the following formula:

$$\begin{matrix}LC = Average of the low control values \\ = Cells treated with 10 μ M of LDC 4297 ( JNJS 64085047 - AAA )\end{matrix}$$

$$\begin{matrix}HC = Average of the high control values \\ = Cells treated with 0.2 \% DMSO\end{matrix}$$

Average value of all HC's and all LC's are used for normalizations.

$$\% Effect = 100 - ( sample - LC ) / ( HC - LC ) \times 100$$

$$\% Control = ( sample / HC ) \times 100$$

A best-fit curve is fitted by a minimum sum of squares method to the plot of % Control vs. compound concentration. From this an IC50 value can be obtained. An estimate of the slope of the plot in terms of the Hill coefficient is also obtained.

In parallel this assay was performed in A549 cells overexpressing CDK7-mutant (C312S) to evaluate the effect of the covalent bond on potency and to screen for potential off-target effects. Mutation of Cysteine to Serine (C312S), a less nucleophilic amino acid, prevents CDK7 inhibitors from covalently binding to CDK7 and from inhibiting CDK7 activity in an irreversible manner. A stably transduced A549 cell pool was generated that overexpresses CDK7 mutant (C312S), but also expresses endogenous CDK7-WT. Covalent binders targeting the cysteine at position 312 will show a shift in potency in A549 cells overexpressing mutant C312S CDK7.

### Proliferation Assay Using OCI-AML3 Cells Overexpressing WT or C312S Mutant CDK7:

### Materials

OCI-AML-3 acute myeloid leukemia cells (DSMZ ACC 582), alpha-MEM (Sigma M4526), fetal bovine serum (BioWest S1810-500), L-glutamine (Sigma G7513), Gentamycin (Life Technologies 15750-037), 96-well plates (Costar, catalogue number 3904), CellTiterGLO reagent (Promega G7573).

To assess anti-proliferative effects, CDK7 inhibitor test compounds were tested in 4-day proliferation assays using two different AML cell lines. The parental OCI-AML-3 cell line was used to generate two OCI-AML-3 cell lines overexpressing either CDK7 WT or CDK7 C312S mutant. Mutation of Cysteine to Serine (C312S), a less nucleophilic amino acid, prevents CDK7 inhibitors from covalently binding to CDK7 and from inhibiting CDK7 activity in an irreversible manner.

OCI-AML-3 cells were propagated in alpha-MEM supplemented with 20% heat inactivated fetal bovine serum, 2 mM L-glutamine and 50 μg/ml Gentamycin. Cells were kept between 0.5-2.5 million cells per mL during culturing. Cell passage numbers were not exceeding 30. To assess anti-proliferative effects, 3000 cells were seeded in 135 μL medium per well of a 96-well plate. Compounds were diluted in DMSO at 500× of the desired final concentrations. A pre-dilution of 1/50 of the compounds was prepared in culture medium. 15 μL of these pre-diluted compounds was added per well of 96-well plates. Cells were incubated for 4 days at 37° C. and 5% CO₂. Cell plating numbers were chosen based on growth curves to ensure linear cell growth. After 4 days of incubation 75 μL CellTiterGLO reagent was added to each well. After 10 min of incubation with shaking at 500 rpm at room temperature, luminescence was measured on the Envision multimode plate reader (Perkin Elmer). Covalent binders targeting the cysteine at position 312 will show a shift in potency in OCI-AML3 cells overexpressing mutant C312S CDK7.

IC₅₀ values were calculated using the following formula (Z prime should be >0.5):

$$\begin{matrix}LC = median of the low control values \\ = Low control : Reaction without cells\end{matrix}$$

$$\begin{matrix}HC = Median of the High control values \\ = High control : Reaction with cells without compound\end{matrix}$$

$$\begin{matrix}\% Effect = 100 - ( sample - LC ) / ( HC - LC ) \times 100 \\ \% Control = ( sample / HC ) \times 100 \\ \% Controlmin = ( sample - LC ) / ( HC - LC ) \times 100\end{matrix}$$

A best-fit curve was fitted by a minimum sum of squares method to the plot of % Control vs. compound concentration. From this an IC₅₀ value (inhibitory concentration causing 50% cytotoxicity) can be obtained. An estimate of the slope of the plot in terms of the Hill coefficient was also obtained.

Data for the compounds of the invention in the above assays are provided in Tables A, B, and C (the values in Table are averaged values over all measurements on all batches of a compound; ‘n.c.’ means not calculated).

TABLE A
Cellular assays results with the
compounds of the present disclosure

| Com- | CDK7_A549 | CDK7_A549 |
|---|---|---|
| pound | Wild-type CDK7 | mutant |
| # | (pIC50) | hCDK7(pIC50) |
| 1 | 6.88 | ~5.07 |
| 2 | 6.1 | <5 |
| 3 | 6.89 | <5 |
| 4 | 6.84 | <5 |
| 5 | <5 | <5 |
| 6 | ~5.03 | <5 |
| 7 | 6.84 | <5 |
| 8 | 6.92 | <5 |
| 9 | 6.35 | <5 |
| 10 | 6.69 | <5 |
| 11 | 7.06 | <5 |
| 12 | 6.27 | <5 |
| 13 | 6.87 | <5 |
| 14 | 6.4 | <5 |
| 15 | 7.26 | <5 |
| 16 | 7.34 | 5.42 |
| 17 | 6.9 | <5 |
| 18 | 7 | 5.37 |
| 19 | 6.78 | <5 |
| 20 | 7.23 | <5 |
| 21 | 7.34 | <5 |
| 22 | 7.25 | <5 |
| 23 | 6.92 | <5 |
| 24 | 7.28 | 5.16 |
| 25 | 6.05 | <5 |
| 26 | 7.65 | ~5.1 |
| 27 | 5.43 | <5 |
| 28 | 6.59 | <5 |
| 29 | 7.41 | ~5.11 |
| 30 | ~5.12 | <5 |
| 31 | 6.36 | <5 |
| 32 | 6.4 | <5 |
| 33 | 6.22 | <5 |
| 34 | 6.81 | <5 |
| 35 | 6.16 | <5 |
| 36 | 6.89 | 5.04 |
| 37 | 6.07 | <5 |
| 38 | 6.8 | <5 |
| 39 | 6.85 | <5 |
| 40 | 5.95 | <5 |
| 42 | 6.91 | <5 |
| 43 | 6.47 | <5 |
| 44 | >8.01 | 6.08 |
| 45 | 5.94 | <5 |
| 46 | 6.2 | <5 |
| 47 | 6.06 | <5 |
| 48 | 5.79 | <5 |
| 49 | 7.27 | ~5 |
| 50 | ~6.75 | <5 |
| 51 | 6.38 | <5 |
| 52 | 6.55 | 5.55 |
| 53 | 6.73 | <5 |
| 54 | 6.84 | 5.09 |
| 55 | <5 | <5 |
| 56 | 6.74 | <5 |
| 57 | 5.92 | <5.4 |
| 58 | 6.31 | <5 |
| 59 | 7.14 | <5.4 |
| 60 | 6.01 | <5 |
| 61 | 5.79 | <5 |
| 62 | 7.11 | <5 |
| 63 | 6.54 | <5 |
| 64 | <5 | <5 |
| 65 | 7.17 | 5.06 |
| 66 | 7.16 | <5 |
| 67 | 6.67 | <5 |
| 68 | 7.4 | 5.14 |
| 69 | 6.63 | <5 |
| 70 | 6.84 | <5 |
| 71 | 6.61 | <5 |
| 72 | 6.23 | <5 |
| 73 | 7.32 | <5 |
| 74 | 7.11 | <5 |
| 75 | 6.61 | <5 |
| 76 | 7.1 | <5 |
| 77 | 6.74 | <5 |
| 78 | 7.57 | 5.09 |
| 79 | 7.43 | 5.23 |
| 80 | 6.38 | <5 |
| 81 | 6.38 | <5 |
| 82 | 7.33 | 5.1 |
| 83 | 6.95 | <5 |
| 84 | 6.24 | <5 |
| 85 | 7.09 | 5.36 |
| 86 | 5.8 | <5 |
| 87 | 7.09 | <5 |
| 88 | 6.79 | <5 |
| 89 | 7.22 | <5 |
| 91 | 7.42 | <5 |
| 92 | 6.9 | <5 |
| 93 | 6.35 | <5 |
| 94 | <5 | <5 |
| 95 | 6.54 | <5 |
| 96 | 6.91 | <5 |
| 97 | 7.18 | <5 |
| 98 | 5.9 | <5 |
| 99 | 7.35 | <5 |
| 100 | 7.29 | 5.48 |
| 101 | 7.19 | <5 |
| 102 | 7.39 | 5.45 |
| 103 | 6.9 | <5 |
| 104 | 5.93 | <5 |
| 105 | 7.79 | 5.59 |
| 106 | ~5 | <5 |
| 107 | 6.96 | <5 |
| 108 | 6.03 | <5 |
| 109 | 5.55 | <5 |
| 110 | 6.59 | <5 |
| 111 | 7.34 | 5.38 |
| 112 | 6.81 | 5.07 |
| 113 | 6.42 | <5 |
| 114 | 7.3 | <5 |
| 115 | 6.66 | <5 |
| 116 | 6.4 | <5 |
| 117 | 6.9 | 5.11 |
| 118 | 7.15 | <5 |
| 119 | 7.42 | <5 |
| 120 | 6.48 | <5 |
| 121 | 6.9 | <5 |
| 122 | 7.16 | <5 |
| 123 | 7.15 | <5 |
| 124 | 7.01 | <5 |
| 125 | 7.56 | 5.14 |
| 126 | 7.32 | <5 |
| 127 | 7.56 | 5.08 |
| 128 | 5.35 | <5 |
| 129 | 6.82 | <5 |
| 130 | 7.01 | <5 |
| 131 | 5.18 | <5 |
| 132 | 6.44 | <5 |
| 133 | 6.78 | <5 |
| 134 | 7.42 | ~5.06 |
| 135 | 6.58 | ~5.15 |
| 136 | 7.14 | 5.14 |
| 137 | 6.9 | <5 |
| 138 | 7.54 | ~5.43 |
| 139 | 6.18 | <5 |
| 140 | 6.93 | <5 |
| 141 | 6.75 | <5 |
| 142 | 5.84 | <5 |
| 143 | 6.91 | <5 |
| 144 | <5 | <5 |
| 145 | 6.01 | <5 |
| 146 | 6.67 | <5 |
| 147 | 6.54 | <5 |
| 148 | 7.43 | 5.26 |
| 149 | 7.3 | <5 |
| 150 | 7.33 | 5.22 |
| 151 | 7.46 | <5 |
| 152 | ~7.17 | <5 |
| 153 | 7.73 | 5.24 |
| 154 | 6.93 | <5 |
| 155 | 6.3 | <5 |
| 156 | 6.31 | <5 |
| 157 | <5 | <5 |
| 158 | 6.9 | <5.4 |
| 159 | 7.15 | <5 |
| 160 | 7.19 | <5 |
| 161 | 6.91 | <5 |
| 162 | 6.74 | <5 |
| 164 | 7.26 | <5 |
| 165 | 7.24 | 5.22 |
| 166 | 7.12 | <5 |
| 167 | 6.98 | <5 |
| 168 | 7.11 | <5 |
| 169 | 7.27 | <5 |
| 170 | 6.92 | 5.11 |
| 173 | 7.19 | <5.4 |
| 174 | 6.49 | <5 |
| 175 | 6.62 | <5 |
| 176 | 6.73 | <5 |
| 177 | 7.05 | <5 |
| 178 | 7.65 | 5.17 |
| 181 | ~7.68 | <5 |
| 182 | 7.42 | <5 |
| 183 | 7.25 | <5 |
| 184 | 7.49 | <5 |
| 185 | 7.09 | <5 |
| 186 | 7 | <5 |
| 187 | 5.39 | <5 |
| 188 | 5.97 | <5 |
| 189 | 7.37 | <5 |
| 190 | 7.47 | ~5.05 |
| 191 | 5.36 | <5 |
| 192 | 7.1 | <5 |
| 193 | 7.16 | <5 |
| 194 | 7.23 | <5 |
| 195 | 6.89 | <5 |
| 196 | 6.98 | <5.4 |
| 197 | 6.91 | <5 |
| 198 | 6.62 | <5 |
| 199 | 6.54 | <5 |
| 200 | 7.03 | ~5 |
| 201 | 7.05 | ~5 |
| 202 | 6.97 | <5 |
| 203 | 7.09 | <5 |
| 204 | 6.47 | <5 |
| 205 | 7.03 | <5 |
| 206 | 7.03 | <5 |
| 207 | 7.08 | <5 |
| 209 | 7 | <5 |
| 210 | 5.07 | <5 |
| 211 | ~5 | <5 |
| 212 | 6.4 | <5 |
| 213 | 6.6 | <5 |
| 214 | 7 | <5 |
| 215 | 7.19 | <5 |
| 216 | 6.26 | <5 |
| 217 | 7.15 | <5 |
| 218 | 5.07 | <5 |
| 219 | 6.7 | <5 |
| 220 | 6.2 | <5 |
| 221 | 6.55 | <5 |
| 222 | 7.01 | <5 |
| 223 | 7.38 | 5.12 |
| 224 | 5.4 | <5 |
| 225 | 6.9 | <5 |
| 226 | 6.55 | <5 |
| 227 | 6.14 | <5 |
| 228 | 6.75 | <5 |
| 229 | 5.65 | <5 |
| 230 | 7.7 | 5.17 |
| 231 | 6.42 | <5 |
| 232 | 5.38 | <5 |
| 233 | 7.55 | 5.06 |
| 234 | 7.02 | 5.07 |
| 235 | 7.15 | 5.02 |
| 236 | 6.79 | 5.13 |
| 237 | 6.88 | <5 |
| 238 | 7.39 | 5.48 |
| 239 | 7.36 | 5.15 |
| 240 | 7.26 | 5.29 |
| 241 | 7.02 | 4.97 |
| 242 | 6.5 | <5 |
| 243 | 6.9 | <5 |
| 244 | 7.14 | ~5.1 |
| 245 | 7.25 | <5 |
| 246 | ~7.54 | <5 |
| 247 | 6.11 | <5.4 |
| 248 | 6.76 | <5 |
| 250 | 5.74 | <5 |
| 251 | 7.36 | <5 |
| 252 | 5.6 | <5 |
| 253 | 7.3 | 5.2 |
| 254 | 6.97 | <5 |
| 255 | 5.52 | <5 |
| 256 | 7.27 | 5.08 |
| 257 | 7.35 | 5.14 |
| 258 | 7.67 | 5.19 |
| 259 | <5 | <5 |
| 260 | 7.1 | <5 |
| 261 | 6.02 | <5.4 |
| 262 | 7.53 | <5 |
| 263 | 7.29 | <5.4 |
| 264 | ~6.86 | <5 |
| 265 | 5.68 | <5 |
| 266 | ~4.99 | <5 |
| 267 | 7.63 | ~5.14 |
| 268 | 5.97 | <5 |
| 269 | 6.79 | <5 |
| 270 | 6.93 | <5 |
| 271 | 5.5 | <5 |
| 272 | 7.35 | <5 |
| 273 | 6.94 | <5 |
| 274 | 6.76 | <5 |
| 275 | 7.06 | <5 |
| 276 | 6.71 | <5 |
| 278 | 6.78 | <5 |
| 279 | 6.86 | <5 |
| 280 | 6.9 | <5 |
| 281 | 6.16 | <5 |
| 282 | 7.51 | <5 |
| 283 | 7.16 | <5 |
| 284 | 5.69 | <5 |
| 285 | 7.71 | 5.73 |
| 286 | 6.9 | <5 |
| 287 | 6.89 | <5 |
| 288 | 6.89 | 5 |
| 289 | 6.91 | <5 |
| 290 | 6.53 | <5 |
| 291 | 5.91 | <5 |
| 292 | 7.25 | 5.08 |
| 293 | 6.99 | <5 |
| 294 | 7.24 | <5 |
| 295 | 6.75 | ~5.08 |
| 296 | 6.65 | <5 |
| 297 | ~7.63 | <5 |
| 298 | 6.58 | <5 |
| 299 | 7.06 | <5 |
| 301 | 7.62 | <5 |
| 302 | ~5 | <5 |
| 303 | 7.04 | 5.21 |
| 304 | 6.89 | <5 |
| 305 | 6.62 | <5 |
| 306 | 6.91 | <5 |
| 307 | <5 | <5 |
| 308 | 6.35 | <5 |
| 309 | 6.89 | <5 |
| 310 | 7.12 | <5 |
| 311 | <5 | <5 |
| 312 | <5 | <5 |
| 313 | 7.11 | 5.27 |
| 314 | 7.14 | <5 |
| 315 | 7.25 | 5.3 |
| 316 | 6.97 | 5.32 |
| 317 | 7.22 | <5 |
| 318 | 6.49 | <5 |
| 319 | 7.11 | <5 |
| 320 | <5 | <5 |
| 321 | 5.03 | 5.06 |
| 322 | 7.1 | 5.02 |
| 323 | 7.08 | 5.1 |
| 325 | >8.01 | 5.38 |
| 326 | 7.05 | 5.11 |
| 327 | 7.1 | ~5 |
| 328 | 6.65 | <5 |
| 329 | 7.1 | <5 |
| 330 | 5.14 | <5 |
| 332 | 5.12 | <5 |
| 333 | 6.75 | 5.17 |
| 334 | 5.28 | <5 |
| 335 | 7.26 | 5.44 |
| 336 | 7.31 | 5.88 |
| 337 | <5.3 | <5 |
| 338 | 7.25 | 5.52 |
| 339 | 6.84 | <5 |
| 340 | 7.37 | 5.43 |
| 341 | 7.35 | 5.64 |
| 342 | 7.85 | 5.18 |
| 343 | 7.15 | <5 |
| 344 | 5.4 | <5 |
| 345 | 7.06 | <5 |
| 346 | 6.72 | <5 |
| 347 | 7.32 | 5.29 |
| 348 | 7.32 | 5.51 |
| 349 | 7.01 | 5.08 |
| 350 | 7.09 | 5 |
| 351 | 6.8 | <5 |
| 352 | 7.52 | 5.51 |
| 353 | 6.68 | <5 |
| 354 | 7.82 | 5.34 |
| 355 | 6.71 | <5 |
| 356 | 5.11 | <5 |
| 357 | 7.24 | 5.11 |
| 358 | 7.4 | 5.21 |
| 359 | 6.99 | 5.55 |
| 360 | 6.17 | <5 |
| 361 | 7.22 | ~5 |
| 362 | 7.23 | 5 |
| 363 | 7.52 | <5 |
| 364 | 6.79 | <5 |
| 365 | 7.36 | <5 |
| 366 | 6.68 | 4.97 |
| 367 | 7.12 | 4.98 |
| 368 | 6.72 | <5 |
| 369 | 6.61 | <5 |
| 370 | 7.19 | <5 |
| 371 | 5.66 | <5 |
| 372 | 7.15 | <5 |
| 373 | 6.57 | <5 |
| 374 | 6.34 | <5 |
| 375 | 5.89 | <5 |
| 376 | 7.45 | 5.3 |
| 377 | 7.3 | 5.24 |
| 378 | 6.79 | <5.4 |
| 379 | 6.65 | 5.15 |
| 380 | 6.79 | <5 |
| 381 | 6.43 | 5.16 |
| 382 | 6.69 | <5 |
| 383 | <5 | <5 |
| 384 | 6.45 | 5.29 |
| 385 | 6.8 | 5.37 |
| 386 | 5.79 | <5 |
| 387 | 6.01 | <5 |
| 388 | ~7.64 | 5.14 |
| 389 | 6.85 | <5 |
| 390 | 6.67 | 5.06 |
| 391 | ~7.21 | <5 |
| 392 | 6.89 | ~5 |
| 393 | 6.98 | <5 |
| 394 | 6.47 | <5 |
| 395 | <5 | <5 |
| 396 | 7.03 | <5 |
| 397 | <5 | <5 |
| 398 | 7.52 | 5.21 |
| 399 | 6.35 | <5 |
| 400 | 6.98 | <5 |
| 401 | 5.17 | <5 |
| 402 | 7.1 | 5.15 |
| 403 | 6.81 | <5 |
| 404 | 7.13 | 5.09 |
| 405 | 7.05 | <5 |
| 406 | ~8.01 | 5.74 |
| 407 | 5.72 | 5.11 |
| 408 | <5 | <5 |
| 409 | 5.84 | 5.15 |
| 410 | 6.76 | <5 |
| 411 | 6.54 | <5 |
| 412 | 5.01 | <5 |
| 413 | 7.12 | <5 |
| 414 | 6.6 | <5 |
| 415 | 5.74 | <5 |
| 416 | >8.01 | 5.73 |
| 417 | 7.23 | 5.53 |
| 418 | 6.59 | 5.06 |
| 421 | 7.02 | 5 |
| 422 | 6.94 | <5 |
| 423 | 7.23 | 5.23 |
| 425 | 6.7 | <5 |
| 426 | 7.2 | <5 |
| 427 | 6.99 | <5 |
| 430 | 6.03 | <5 |
| 431 | 7.26 | <5 |
| 432 | <5 | <5 |
| 433 | 6.77 | 5.27 |
| 434 | 5.34 | <5 |
| 435 | 7.16 | <5 |
| 436 | 6.24 | <5 |
| 437 | 6.77 | <5 |
| 438 | 6.72 | 5.51 |
| 439 | 5.96 | <5 |
| 440 | 6.79 | <5 |
| 441 | <5 | <5 |
| 442 | 6.25 | <5 |
| 443 | 7.69 | 5.37 |
| 445 | 6.91 | 5.07 |
| 446 | 6.1 | <5 |
| 447 | 6.77 | ~5 |
| 448 | 6.68 | 5.08 |
| 449 | 7.48 | 5.63 |
| 450 | 5.75 | <5 |
| 451 | 6.52 | <5 |
| 452 | 7.19 | 5.16 |
| 453 | 7.13 | 5.54 |
| 454 | 6.59 | <5 |
| 455 | 7.04 | <5 |
| 456 | 5.54 | <5 |
| 457 | 7.04 | <5 |
| 458 | 5.28 | <5 |
| 459 | 7.14 | 5.36 |
| 460 | 5.89 | <5 |
| 461 | 6.16 | <5 |
| 462 | 7.3 | 5.28 |
| 463 | 5.98 | <5 |
| 464 | 5.12 | <5 |
| 465 | 7.14 | 5.15 |
| 466 | 6.91 | <5 |
| 467 | 6.93 | <5 |
| 468 | 5.69 | <5 |
| 469 | 5.67 | <5 |
| 470 | 6.6 | <5 |
| 471 | 5.15 | <5 |
| 472 | 6.69 | <5 |
| 473 | 6.78 | <5 |
| 474 | 6.24 | <5 |
| 475 | 6.69 | 5.12 |
| 476 | 6.26 | <5 |
| 477 | 6.78 | <5 |
| 478 | 6.8 | <5 |
| 479 | <5 | <5 |
| 480 | 5.34 | <5 |
| 481 | 5.52 | <5 |
| 482 | 7.05 | <5 |
| 484 | 6.85 | <5 |
| 485 | 6.92 | <5 |
| 486 | 5.25 | <5 |
| 487 | <5 | <5 |
| 488 | ~6.52 | <5 |
| 489 | 7.23 | 5.46 |
| 490 | 7.13 | 5.48 |
| 491 | 6.32 | <5 |
| 492 | 6.9 | 5.02 |
| 493 | 6.92 | 5.23 |
| 494 | 5.39 | <5 |
| 495 | 6.84 | <5 |
| 496 | 7.85 | 5.14 |
| 497 | 6.86 | <5 |
| 498 | 6.83 | <5 |
| 499 | 5.74 | <5 |
| 500 | 5.92 | <5 |
| 501 | 5.78 | <5 |
| 502 | 6.22 | <5 |
| 503 | 6.88 | 5.12 |
| 504 | 7.32 | 5.11 |
| 505 | 7.42 | 5.62 |
| 506 | 6.99 | 5.08 |
| 507 | 6.96 | 5.09 |
| 508 | 5.54 | <5 |
| 509 | 5.62 | <5 |
| 510 | 6.72 | <5 |
| 511 | 6.94 | 5.25 |
| 512 | 7.56 | <5 |
| 513 | 6.36 | <5 |
| 514 | 6.49 | <5 |
| 515 | ~7.94 | 5.05 |
| 516 | 5.68 | <5 |
| 517 | 5.52 | <5 |
| 518 | 7.19 | 5.39 |
| 519 | 5.23 | <5 |
| 520 | 6.8 | <5 |
| 521 | 5.28 | <5 |
| 522 | 6.64 | <5 |
| 523 | 7.01 | <5 |
| 524 | 5.03 | <5 |
| 525 | 6.85 | 5.27 |
| 526 | 7.85 | <5.6 |
| 527 | 5.01 | <5 |
| 528 | 6.22 | <5 |
| 529 | 5.76 | <5 |
| 530 | <5 | <5 |
| 531 | <5 | <5 |
| 532 | 6.37 | 4.99 |
| 533 | 6.33 | <5 |
| 534 | ~5.02 | <5 |
| 535 | 7.2 | ~5 |
| 536 | <5 | <5 |
| 537 | <5 | <5 |
| 538 | 6.49 | <5 |
| 539 | 5.88 | <5 |
| 540 | 6.26 | <5 |
| 541 | 6.81 | <5 |
| 542 | 6.62 | <5 |
| 543 | 5.92 | <5 |
| 544 | 5.53 | <5 |
| 545 | 6.93 | 5.55 |
| 546 | 5.56 | <5 |
| 547 | <5 | <5 |

TABLE B
Biochemical and enzymatic assay results
with the compounds of the present disclosure

| | | | CDK7 |
|---|---|---|---|
| compound | CDK7 | CDK7 | (kinact/KI, |
| # | (KI(μM)) | (kinact, s⁻¹) | M⁻¹*s⁻¹) |
| 1 | 0.1067 | 0.0002997 | 2027.41 |
| 2 | 0.1292 | 0.00007862 | 608.514 |
| 3 | | | 2787.5 |
| 4 | 0.06172 | 0.0003033 | 3147.06 |
| 7 | | | 1065.34 |
| 8 | | | 1528 |
| 9 | | | 486.4 |
| 10 | 0.05549 | 0.0001066 | 1571 |
| 11 | | | 687.5 |
| 12 | | | 296.5 |
| 13 | | | 1044.78 |
| 14 | | | 710 |
| 15 | | | 320.6 |
| 16 | | | 402 |
| 19 | | | 304.35 |
| 22 | | | 52.362 |
| 23 | | | 455.169 |
| 24 | | | 21.5216 |
| 25 | 0.0648305 | 2.33019E−05 | 254.073 |
| 26 | 0.0629884 | 0.000102094 | 1620.84 |
| 27 | | | 28.6597 |
| 28 | | | 476.982 |
| 30 | 0.316033 | 6.55994E−05 | 207.572 |
| 31 | 0.175114 | 0.000111744 | 638.124 |
| 32 | 0.176749 | 0.000102239 | 578.444 |
| 33 | 0.150271 | 0.000157939 | 1051.03 |
| 34 | | | 2083.98 |
| 35 | 0.122612 | 0.000133195 | 1086.32 |
| 37 | 0.129393 | 0.000109859 | 849.028 |
| 38 | | | 744.112 |
| 39 | | | 803.227 |
| 40 | 0.209426 | 6.41303E−05 | 306.22 |
| 41 | 0.083 | 0.000083668 | 1008.05 |
| 42 | 0.0810955 | 0.00006169 | 760.708 |
| 43 | 0.0497295 | 5.45507E−05 | 1096.95 |
| 44 | | | 601.977 |
| 45 | 0.123364 | 0.000137313 | 1113.07 |
| 46 | 0.22289 | 0.000101695 | 456.258 |
| 47 | 0.207092 | 0.000125353 | 605.302 |
| 48 | 0.325071 | 4.97915E−05 | 153.171 |
| 49 | | | 990.301 |
| 50 | 0.132211 | 0.000144175 | 1090.49 |
| 51 | 0.0705981 | 7.83893E−05 | 1110.36 |
| 52 | 0.165967 | 0.000165672 | 998.221 |
| 53 | | | 772.776 |
| 54 | 0.100454 | 0.000100109 | 996.563 |
| 55 | | | 2.44583 |
| 56 | 0.0672586 | 6.99314E−05 | 1039.74 |
| 57 | 0.248313 | 0.00012856 | 517.735 |
| 58 | 0.111746 | 0.000057638 | 515.793 |

TABLE C
Biochemical and enzymatic assay results with
the compounds of the present disclosure

| | CDK7 | CDK7 | CDK7 (20 nM | CDK7 (20 nM |
|---|---|---|---|---|
| compound | (20 nM enz.) | (20 nM enz.) | enz.) (kinact/ | enz.) (kobs/ |
| # | (KI, μM) | (kinact, s⁻¹) | KI, M⁻¹*s⁻¹) | [I], M⁻¹*s⁻¹) |
| 1 | 0.045565 | 5.77473E−05 | 1267.36 | |
| 8 | 0.032051 | 5.80001E−05 | 1809.62 | |
| 14 | 0.129681 | 7.07114E−05 | 545.271 | |
| 16 | 0.0285782 | 3.30339E−05 | 1155.91 | |
| 17 | | | | 1469.63 |
| 18 | | | | 1072.14 |
| 22 | 0.0524205 | 7.21518E−05 | 1871.77 | |
| 24 | | | 1252.9 | |
| 26 | | | | 1343.33 |
| 29 | | | 1028.3 | |
| 34 | | | | 1033.06 |
| 36 | 0.092 | 0.0000492 | 535 | |
| 38 | 0.0618804 | 6.90624E−05 | 1116.06 | |
| 44 | | | | 1572.38 |
| 49 | | | | 1154.15 |
| 59 | | | 1476.3 | |
| 60 | 0.1281 | 0.0000643 | 501.9 | |
| 61 | 1.323 | 4.83859E−05 | 36.5728 | |
| 62 | 0.0266 | 0.0000642 | 2409.8 | |
| 63 | 0.0857 | 7.59019E−05 | 885.2 | |
| 64 | 0.445432 | 1.42743E−05 | 32.0459 | |
| 65 | | | | 1156.2 |
| 66 | | | | 1029.3 |
| 67 | 0.0862 | 6.48096E−05 | 751.5 | |
| 68 | | | | 1369 |
| 69 | | | | 529.4 |
| 70 | | | | 633.9 |
| 71 | 0.0575275 | 5.60004E−05 | 973.456 | |
| 72 | 0.411884 | 6.82131E−05 | 165.612 | |
| 73 | | | 1301.8 | |
| 74 | | | | 1043.01 |
| 75 | | | | 1101.89 |
| 76 | | | | 1645.29 |
| 77 | 0.0571204 | 5.74703E−05 | 1006.13 | |
| 78 | | | | 908.474 |
| 79 | | | | 766.385 |
| 80 | 0.0562681 | 4.16486E−05 | 740.18 | |
| 81 | 0.0670297 | 4.29043E−05 | 640.079 | |
| 82 | | | | 1568.1 |
| 83 | | | | 782.13 |
| 84 | 0.190516 | 6.74114E−05 | 353.837 | |
| 85 | | | | 608.167 |
| 86 | 0.396723 | 0.000025039 | 63.1145 | |
| 87 | | | | 1415 |
| 88 | | | | 1045.31 |
| 89 | | | | 923.077 |
| 90 | | | | 1194.19 |
| 91 | | | | 1740.91 |
| 92 | 0.0776837 | 4.72491E−05 | 622.396 | |
| 93 | 0.0861921 | 5.07244E−05 | 588.505 | |
| 94 | 3.36612 | 3.78528E−05 | 11.2452 | |
| 95 | 0.480368 | 4.87115E−05 | 101.404 | |
| 96 | 0.183719 | 4.70076E−05 | 255.867 | |
| 97 | 0.0626941 | 0.000110045 | 1755.26 | 1510.5 |
| 98 | 0.39042 | 5.61113E−05 | 143.721 | |
| 99 | | | | 1439.84 |
| 100 | 0.0428119 | 5.05122E−05 | 1179.87 | |
| 101 | | | | 1114.68 |
| 102 | 0.0267988 | 4.37942E−05 | 1634.19 | |
| 103 | 0.0423515 | 8.55542E−05 | 2020.1 | |
| 104 | 0.431349 | 4.84629E−05 | 112.352 | |
| 105 | 0.0256438 | 5.79707E−05 | 2260.61 | |
| 107 | 0.0560402 | 5.28289E−05 | 942.696 | |
| 108 | 0.569987 | 5.74776E−05 | 100.84 | |
| 109 | 2.4159 | 1.42535E−05 | 5.89986 | |
| 110 | 0.22983 | 5.83291E−05 | 253.792 | |
| 111 | | | | 622.12 |
| 112 | 0.0653401 | 4.52818E−05 | 693.016 | |
| 113 | 0.389766 | 6.16268E−05 | 158.112 | |
| 114 | 0.0778719 | 4.23027E−05 | 543.235 | |
| 115 | 0.048601 | 5.80228E−05 | 1193.86 | |
| 116 | 0.292536 | 5.05477E−05 | 172.792 | |
| 117 | 0.0511503 | 4.55571E−05 | 890.652 | |
| 118 | | | | 1620.07 |
| 119 | | | | 1611.37 |
| 120 | 0.0783 | 0.0000813 | 1039 | |
| 121 | 0.0963187 | 9.75262E−05 | 1012.54 | |
| 122 | | | | 1655 |
| 123 | 0.0243767 | 3.39754E−05 | 1393.76 | |
| 124 | | | | 1546 |
| 125 | | | | 1227.34 |
| 126 | | | | 875.576 |
| 127 | | | | 1423.45 |
| 128 | 10.1835 | 0.000114966 | 11.2895 | |
| 129 | 0.169713 | 5.47775E−05 | 322.765 | |
| 130 | 0.109869 | 7.73255E−05 | 703.799 | |
| 132 | 0.31198 | 0.000070767 | 226.832 | |
| 133 | 0.0971052 | 6.93886E−05 | 714.571 | |
| 134 | | | | 1525.35 |
| 135 | 0.0578148 | 5.70934E−05 | 987.522 | |
| 136 | 0.0603039 | 4.42381E−05 | 733.587 | |
| 137 | 0.0381961 | 6.40698E−05 | 1677.39 | |
| 138 | | | | 1329.75 |
| 139 | 0.514807 | 6.73145E−05 | 130.757 | |
| 140 | 0.0741172 | 6.51012E−05 | 878.355 | |
| 141 | 0.159824 | 8.35694E−05 | 522.884 | |
| 142 | 0.332268 | 4.75471E−05 | 143.099 | |
| 143 | 0.0403904 | 6.96285E−05 | 1723.89 | |
| 144 | 35.9072 | 0.000162706 | 4.53128 | |
| 145 | 0.871992 | 7.89817E−05 | 90.5762 | |
| 146 | 0.0284633 | 5.52976E−05 | 1942.77 | |
| 147 | 0.0278017 | 5.84022E−05 | 2100.67 | |
| 148 | 0.0507108 | 0.000033794 | 666.405 | |
| 149 | 0.0542369 | 6.65576E−05 | 1227.17 | |
| 150 | 0.05262 | 0.00008759 | 1664.58 | |
| 151 | 0.0205457 | 4.67444E−05 | 2275.14 | |
| 152 | 0.0241841 | 4.68019E−05 | 1935.23 | |
| 153 | 0.0287504 | 3.66796E−05 | 1275.8 | |
| 154 | 0.136657 | 5.98086E−05 | 437.656 | |
| 155 | 2.08784 | 4.59415E−05 | 22.0043 | |
| 156 | 0.303542 | 9.03163E−05 | 297.541 | |
| 158 | 0.203534 | 0.000076792 | 377.294 | |
| 159 | 0.144485 | 0.000114716 | 793.961 | |
| 160 | 0.0392681 | 6.88348E−05 | 1752.94 | |
| 161 | 0.0362971 | 3.08873E−05 | 850.959 | |
| 162 | 0.149114 | 4.87851E−05 | 327.166 | |
| 164 | 0.0785903 | 7.93283E−05 | 1009.39 | |
| 165 | 0.0638365 | 5.15972E−05 | 808.271 | |
| 166 | 0.0261932 | 5.15867E−05 | 1969.47 | |
| 167 | 0.0325031 | 5.40299E−05 | 1662.3 | |
| 169 | 0.0532368 | 3.62959E−05 | 681.781 | |
| 170 | 0.125087 | 3.69643E−05 | 295.508 | |
| 171 | 0.0278023 | 5.15278E−05 | 1853.36 | |
| 172 | 1.40452 | 5.52765E−05 | 39.3562 | |
| 173 | 0.0574101 | 7.26083E−05 | 1264.73 | |
| 174 | 0.143318 | 5.73989E−05 | 400.501 | |
| 175 | 0.0694932 | 4.03237E−05 | 580.255 | |
| 176 | 0.037232 | 5.37843E−05 | 1444.57 | |
| 177 | | | | 1025 |
| 178 | | | | 1906 |
| 179 | 0.0605887 | 5.08408E−05 | 839.113 | |
| 180 | 0.0451942 | 5.34186E−05 | 1181.98 | |
| 181 | | | | 864.055 |
| 182 | 0.0772022 | 8.64743E−05 | 1120 | |
| 183 | 0.0730675 | 5.78162E−05 | 791.3 | |
| 184 | 0.0445974 | 6.02545E−05 | 1351 | |
| 185 | 0.121847 | 6.39896E−05 | 525.2 | |
| 188 | 0.507068 | 0.000030211 | 59.5798 | |
| 189 | | | | 1445 |
| 190 | | | | 1250 |
| 191 | 3.09165 | 4.71565E−05 | 15.2529 | |
| 192 | 0.0907226 | 4.13575E−05 | 455.868 | |
| 193 | 0.0630197 | 3.66393E−05 | 581.395 | |
| 194 | 0.0891584 | 4.72094E−05 | 529.5 | |
| 196 | 0.0924365 | 4.58066E−05 | 495.546 | |
| 197 | 0.133507 | 7.96358E−05 | 596.491 | |
| 198 | 0.365083 | 0.000044674 | 122.366 | |
| 199 | 0.448864 | 3.62777E−05 | 80.8211 | |
| 200 | 0.0506162 | 4.11054E−05 | 812.099 | |
| 201 | 0.0701039 | 3.60072E−05 | 513.626 | |
| 202 | 0.0238954 | 3.54884E−05 | 1485.15 | |
| 203 | 0.0522935 | 4.19455E−05 | 802.116 | |
| 204 | 0.352195 | 3.71252E−05 | 105.411 | |
| 205 | 0.118724 | 4.74407E−05 | 399.588 | |
| 206 | 0.0442882 | 4.44759E−05 | 1004.24 | |
| 207 | 0.0442717 | 3.82862E−05 | 864.801 | |
| 208 | 0.16835 | 4.83603E−05 | 287.26 | |
| 211 | 7.28705 | 7.17704E−05 | 9.84903 | |
| 212 | 0.199945 | 2.94243E−05 | 147.162 | |
| 213 | 0.267497 | 0.000049048 | 183.359 | |
| 214 | | | | 417 |
| 215 | | | | 753 |
| 216 | 0.472276 | 6.65969E−05 | 141.013 | |
| 217 | 0.101135 | 8.54839E−05 | 845.246 | |
| 218 | | | 0.84 | |
| 219 | 0.310582 | 6.38598E−05 | 205.614 | |
| 220 | 0.114154 | 2.73509E−05 | 239.597 | |
| 221 | 0.512188 | 7.78823E−05 | 152.058 | |
| 222 | 0.18005 | 7.49292E−05 | 416.159 | |
| 223 | 0.127269 | 6.10637E−05 | 479.801 | |
| 224 | | | 3 | |
| 225 | 0.337335 | 6.24499E−05 | 185.127 | |
| 226 | 0.6699 | 6.75708E−05 | 100.867 | |
| 227 | 3.01078 | 6.83409E−05 | 22.6987 | |
| 228 | 0.12859 | 6.25242E−05 | 486.229 | |
| 229 | | | 4 | |
| 230 | | | | 791 |
| 231 | 1.61464 | 6.10285E−05 | 37.797 | |
| 232 | 10.1335 | 0.00011485 | 11.3337 | |
| 233 | | | | 1445.75 |
| 234 | | | | 610.385 |
| 235 | | | | 962.5 |
| 236 | 0.0840451 | 3.04593E−05 | 362.415 | |
| 237 | 0.0730518 | 3.41562E−05 | 467.561 | |
| 238 | | | | 758.077 |
| 239 | | | | 1036.5 |
| 240 | 0.0242173 | 3.61385E−05 | 1492.26 | |
| 241 | 0.026922 | 3.71481E−05 | 1379.84 | |
| 242 | 0.129393 | 2.70258E−05 | 208.866 | |
| 243 | 0.0801556 | 3.92638E−05 | 489.845 | |
| 244 | 0.0398157 | 4.36631E−05 | 1096.63 | |
| 245 | 0.0559233 | 3.60907E−05 | 645.361 | |
| 246 | 0.0946303 | 8.39526E−05 | 887.164 | |
| 247 | 1.57359 | 5.48127E−05 | 34.8328 | |
| 248 | 0.306574 | 8.50628E−05 | 277.463 | |
| 249 | | | | 1037.44 |
| 250 | 0.564089 | 1.06355E−05 | 18.8542 | |
| 251 | | | | 1114.7 |
| 252 | 25.5462 | 0.000305534 | 11.9601 | |
| 253 | | | | 988.991 |
| 254 | | | | |
| 255 | 10.6657 | 3.54779E−05 | 3.32637 | |
| 256 | 0.0827499 | 6.71359E−05 | 811.31 | |
| 257 | 0.0574192 | 7.94726E−05 | 1384.08 | |
| 258 | | | | 619.304 |
| 259 | 0.675307 | 3.66333E−05 | 54.2469 | |
| 260 | 0.0256264 | 4.63951E−05 | 1810.44 | |
| 261 | 1.11353 | 3.88514E−05 | 34.8905 | |
| 262 | 0.0322555 | 3.70838E−05 | 1149.69 | |
| 263 | 0.168218 | 0.000051825 | 308.082 | |
| 264 | 0.0439361 | 3.43586E−05 | 782.011 | |
| 267 | | | | 977.051 |
| 268 | 0.933985 | 4.12954E−05 | 44.2142 | |
| 269 | 0.102559 | 4.28121E−05 | 417.437 | |
| 270 | 0.0517502 | 3.69748E−05 | 714.487 | |
| 272 | 0.021155 | 2.13094E−05 | 1007.3 | |
| 273 | 0.0474375 | 4.48555E−05 | 945.57 | |
| 274 | 0.0714245 | 3.44771E−05 | 482.707 | |
| 275 | | | | 920.635 |
| 276 | 0.2315 | 4.06806E−05 | 175.726 | |
| 277 | 0.156452 | 4.48371E−05 | 286.587 | |
| 278 | 0.225727 | 0.000031322 | 138.76 | |
| 279 | 0.233444 | 2.96236E−05 | 126.898 | |
| 281 | 0.68276 | 4.07108E−05 | 59.6268 | |
| 282 | 0.0396554 | 5.00872E−05 | 1263.06 | |
| 283 | 0.186756 | 7.11968E−05 | 381.229 | |
| 284 | 2.00112 | 3.48516E−05 | 17.416 | |
| 285 | | | | 963.646 |
| 286 | 0.146394 | 4.73759E−05 | 323.619 | |
| 287 | 0.188018 | 0.000116961 | 622.074 | |
| 288 | 0.0411232 | 2.54476E−05 | 618.813 | |
| 289 | 0.0627891 | 2.53758E−05 | 404.143 | |
| 290 | 0.0264916 | 3.60756E−05 | 1361.77 | |
| 291 | | | | 26.496 |
| 292 | | | | 802.483 |
| 293 | 0.100523 | 3.66954E−05 | 365.044 | |
| 295 | 0.0688424 | 5.48086E−05 | 796.146 | |
| 296 | 0.06698 | 3.51992E−05 | 525.518 | |
| 297 | | | | 1237.07 |
| 298 | 0.0310767 | 8.92496E−05 | 2871.91 | |
| 299 | 0.0959439 | 0.000058992 | 614.859 | |
| 300 | 2.6277 | 3.59219E−05 | 13.6705 | |
| 301 | | | | 2792.11 |
| 303 | 0.0492227 | 2.34697E−05 | 476.808 | |
| 304 | 0.110887 | 4.09161E−05 | 368.99 | |
| 305 | 0.0839383 | 2.97418E−05 | 354.33 | |
| 306 | | | | 1166.92 |
| 308 | 0.433717 | 1.50743E−05 | 34.756 | |
| 309 | 0.276905 | 5.45434E−05 | 196.975 | |
| 310 | 0.115096 | 6.29416E−05 | 546.863 | |
| 313 | 0.0471399 | 2.01505E−05 | 427.461 | |
| 314 | 0.0654657 | 3.94473E−05 | 602.565 | |
| 315 | | | | 894.649 |
| 316 | 0.0897707 | 2.25547E−05 | 251.248 | |
| 317 | | | | 1062.72 |
| 318 | 0.044125 | 6.19463E−05 | 1403.88 | |
| 319 | 0.047049 | 6.15319E−05 | 1307.82 | |
| 322 | | | | 740.271 |
| 323 | 0.0217276 | 6.41274E−05 | 2951.42 | |
| 324 | 0.0237333 | 5.02644E−05 | 2117.89 | |
| 325 | | | | 1281.87 |
| 326 | | | | 938.94 |
| 327 | 0.0302413 | 4.15856E−05 | 1375.13 | |
| 328 | 0.472078 | 2.77423E−05 | 58.7663 | |
| 329 | 0.024274 | 3.81437E−05 | 1571.38 | |
| 330 | 4.58125 | 8.81891E−05 | 19.25 | |
| 331 | 0.203977 | 6.25014E−05 | 306.413 | |
| 332 | 4.8527 | 2.19011E−05 | 4.51319 | |
| 333 | 0.109798 | 2.11836E−05 | 192.932 | |
| 335 | 0.0383037 | 0.000018613 | 485.934 | |
| 336 | 0.0563796 | 1.29483E−05 | 229.663 | |
| 338 | 0.162474 | 4.85839E−05 | 299.026 | |
| 339 | 0.074639 | 7.81105E−05 | 1046.51 | |
| 340 | 0.0485646 | 2.46452E−05 | 507.473 | |
| 341 | 0.0442699 | 2.90001E−05 | 655.075 | |
| 342 | | | | 633 |
| 343 | 0.0503196 | 5.90205E−05 | 1172.91 | |
| 344 | 17.1449 | 5.40344E−05 | 3.15163 | |
| 345 | 0.131116 | 0.000038839 | 296.219 | |
| 346 | 0.0788728 | 2.05713E−05 | 260.816 | |
| 347 | 0.0359545 | 2.84394E−05 | 790.982 | |
| 348 | 0.0542933 | 5.99063E−05 | 1103.38 | |
| 349 | 0.056299 | 3.25445E−05 | 578.066 | |
| 350 | 0.025016 | 4.40459E−05 | 1760.71 | |
| 351 | 0.0451683 | 5.23059E−05 | 1158.02 | |
| 352 | 0.0550073 | 5.53024E−05 | 1005.36 | |
| 353 | 4.52411 | 0.00013213 | 29.2058 | |
| 354 | | | | 2669.74 |
| 355 | 0.211499 | 3.50639E−05 | 165.787 | |
| 357 | | | | 2368.08 |
| 358 | 0.0308256 | 3.82893E−05 | 1242.13 | |
| 359 | 0.0280204 | 2.78769E−05 | 994.882 | |
| 360 | 1.54377 | 0.000037136 | 24.0554 | |
| 361 | 0.0290915 | 0.000050565 | 1738.14 | |
| 362 | 0.0324851 | 6.59441E−05 | 2029.98 | |
| 363 | 0.0271941 | 5.09658E−05 | 1874.15 | |
| 364 | | | | 464.289 |
| 365 | 0.046022 | 4.07081E−05 | 884.536 | |
| 366 | | | | 338.632 |
| 367 | 0.0482219 | 3.36658E−05 | 698.144 | |
| 368 | 0.103277 | 0.000044292 | 428.866 | |
| 369 | 0.0216148 | 3.00215E−05 | 1388.93 | |
| 370 | 0.112234 | 2.31743E−05 | 206.483 | |
| 372 | 0.0620837 | 3.74455E−05 | 603.146 | |
| 373 | 0.112837 | 1.65669E−05 | 146.821 | |
| 374 | 1.36326 | 3.18091E−05 | 23.333 | |
| 375 | 2.11627 | 2.57762E−05 | 12.18 | |
| 376 | 0.0466535 | 9.40925E−05 | 2016.84 | |
| 377 | 0.0468154 | 2.19686E−05 | 469.26 | |
| 378 | | | | 1741.94 |
| 379 | 0.0332538 | 1.57835E−05 | 474.639 | |
| 380 | 0.240714 | 4.26846E−05 | 177.325 | |
| 382 | 3.26042 | 4.69806E−05 | 14.4094 | |
| 384 | 0.0300451 | 7.48597E−06 | 249.158 | |
| 385 | 0.0210829 | 1.20077E−05 | 569.548 | |
| 386 | 0.399033 | 1.64042E−05 | 41.1098 | |
| 387 | 0.450888 | 2.21457E−05 | 49.1158 | |
| 388 | | | | 1270.35 |
| 389 | 0.0199078 | 2.13441E−05 | 1072.15 | |
| 390 | 0.04314 | 1.29398E−05 | 299.949 | |
| 391 | 0.0204687 | 2.25223E−05 | 1100.33 | |
| 392 | 0.0655157 | 5.39515E−05 | 823.489 | |
| 393 | 0.047692 | 2.17743E−05 | 456.561 | |
| 394 | 0.313423 | 1.25127E−05 | 39.9227 | |
| 396 | 0.0642578 | 0.000051835 | 806.672 | |
| 398 | | | | 1628.78 |
| 399 | 0.0838682 | 6.6103E−06 | 78.8177 | |
| 400 | 0.0913928 | 5.01794E−05 | 549.051 | |
| 401 | 10.3935 | 6.38272E−05 | 6.14107 | |
| 402 | 0.0823483 | 0.000126015 | 1530.27 | |
| 403 | 0.0766026 | 4.63426E−05 | 604.974 | |
| 404 | | | | 1280.59 |
| 405 | 0.135026 | 6.05825E−05 | 448.672 | |
| 406 | 0.494914 | 0.000181063 | 365.847 | |
| 410 | 0.203338 | 6.11001E−05 | 300.486 | |
| 411 | 3.39793 | 0.000106302 | 31.2844 | |
| 413 | 0.0373794 | 4.94273E−05 | 1322.32 | |
| 414 | 0.0610861 | 4.36281E−05 | 714.207 | |
| 415 | 2.21712 | 7.43236E−05 | 33.5225 | |
| 416 | | | | 1747.95 |
| 417 | 0.0222578 | 1.50884E−05 | 677.896 | |
| 418 | 0.0691511 | 2.86952E−05 | 414.964 | |
| 419 | 0.273394 | 3.95045E−05 | 144.497 | |
| 420 | 7.91951 | 4.54555E−05 | 5.73969 | |
| 421 | 0.0639972 | 7.58789E−05 | 1185.66 | |
| 422 | 0.0415391 | 4.59861E−05 | 1107.06 | |
| 423 | 0.0379975 | 4.88645E−05 | 1286 | |
| 425 | 0.0971416 | 0.00002481 | 255.401 | |
| 426 | 0.641518 | 4.42503E−05 | 68.9774 | |
| 427 | 0.0447454 | 7.83869E−05 | 1751.84 | |
| 429 | 0.0671569 | 9.76008E−05 | 1453.32 | |
| 430 | 1.53612 | 1.08904E−05 | 7.08954 | |
| 431 | | | | 1165.39 |
| 432 | 2.20433 | 2.46707E−05 | 11.1919 | |
| 433 | 0.185195 | 1.88721E−05 | 101.904 | |
| 435 | | | | 856.41 |
| 436 | 0.310793 | 4.29961E−05 | 138.343 | |
| 437 | 0.0822177 | 2.31492E−05 | 281.561 | |
| 439 | 0.400946 | 0.000010115 | 25.2279 | |
| 440 | 0.204699 | 0.000050424 | 246.333 | |
| 442 | 0.77214 | 3.46274E−05 | 44.8461 | |
| 443 | | | | 1431.9 |
| 444 | 0.185693 | 4.50574E−05 | 242.644 | |
| 445 | 0.0843652 | 6.94225E−05 | 822.881 | |
| 447 | 0.142882 | 7.88217E−05 | 551.654 | |
| 449 | | | | 328.213 |
| 452 | 0.0493134 | 4.91791E−05 | 997.278 | |
| 453 | 0.0717162 | 3.15329E−05 | 439.69 | |
| 454 | 0.244694 | 5.68096E−05 | 232.165 | |
| 455 | | | 178.755 | |
| 456 | 24.5665 | 0.000114235 | 4.65005 | |
| 457 | 0.21368 | 0.000103636 | 485.007 | |
| 458 | 19.171 | 6.89137E−05 | 3.59468 | |
| 459 | | | | 734.255 |
| 460 | | | 14.4456 | |
| 461 | | | 16.0518 | |
| 462 | | | 509 | |
| 463 | 0.42283 | 3.43556E−05 | 81.2516 | |
| 464 | 4.0313 | 2.27476E−05 | 5.64276 | |
| 465 | 0.0328686 | 2.78787E−05 | 848.186 | |
| 466 | 0.0307349 | 5.01698E−05 | 1632.34 | |
| 467 | 0.0121274 | 3.18468E−05 | 2626.03 | |
| 468 | 2.21153 | 3.63913E−05 | 16.4553 | |
| 469 | 1.32258 | 4.02779E−05 | 30.454 | |
| 470 | 0.0393951 | 0.000029986 | 761.16 | |
| 471 | 3.63815 | 3.28311E−05 | 9.02413 | |
| 472 | | | 79.4947 | |
| 475 | | | 351.068 | |
| 476 | 0.235833 | 2.18156E−05 | 92.5047 | |
| 477 | | | 341.805 | |
| 478 | | | 292.296 | |
| 480 | | | 2.97344 | |
| 482 | | | 338.938 | |
| 484 | | | 288.798 | |
| 486 | | | 1.32216 | |
| 487 | | | 1.53653 | |
| 488 | 0.328286 | 4.23383E−05 | 128.968 | |
| 489 | | | 305.041 | |
| 490 | | | 472.919 | |
| 491 | 0.403363 | 2.47866E−05 | 61.4499 | |
| 493 | | | 397.303 | |
| 494 | | | 3.13865 | |
| 495 | 0.0762943 | 5.75341E−05 | 754.107 | |
| 496 | | | | 679.724 |
| 497 | 0.0436898 | 5.54737E−05 | 1269.72 | |
| 498 | 0.0866884 | 5.15131E−05 | 594.233 | |
| 499 | 2.1938 | 4.48224E−05 | 20.4314 | |
| 500 | | | 12.6656 | |
| 501 | 2.35375 | 3.59393E−05 | 15.2689 | |
| 502 | | | 9.737 | |
| 503 | | | 629.38 | |
| 504 | | | 680.784 | |
| 505 | | | 245.706 | |
| 506 | | | | 347.826 |
| 507 | | | | 388.747 |
| 508 | | | 6.21048 | |
| 509 | 1.27771 | 1.85034E−05 | 14.4817 | |
| 510 | 0.0519889 | 3.09236E−05 | 594.812 | |
| 517 | 2.915 | 0.0000423 | 14.3 | |
| 518 | 0.0075 | 0.0000326 | 4339 | |
| 519 | 6.24 | 0.0000414 | 6.63 | |
| 520 | 0.00977 | 0.0000291 | 2981 | |
| 521 | 1.711 | 0.0000272 | 15.9 | |
| 522 | 0.007785 | 0.0000165 | 2122 | |
| 523 | 0.0496 | 0.0000197 | 397.8 | |
| 525 | 0.0109 | 0.0000144 | 1322 | |
| 526 | 0.0767 | 0.0000261 | 339.9 | |
| 527 | 1.811 | 0.0000158 | 8.74 | |
| 528 | 0.0976 | 0.000017 | 174.4 | |
| 529 | 0.0408 | 0.0000166 | 408.7 | |
| 530 | 5.62 | 0.0000147 | 2.83 | |
| 531 | 33.1 | 0.000029 | 0.88 | |
| 532 | 0.0823 | 0.0000152 | 184 | |
| 533 | 0.135 | 0.000032 | 236.1 | |
| 536 | 6.8 | 2.49E-11 | 3.67E−12 | |
| 537 | 0.003 | 2.55E-11 | 0.0000037 | |
| 538 | 0.0346 | 0.0000258 | 746.2 | |
| 539 | 0.01959 | 3.05E-12 | 0.000157 | |
| 540 | 0.0301 | 0.0000715 | 2312 | |
| 541 | 0.0182 | 0.0000494 | 2713 | |
| 542 | 0.0082 | 0.0000556 | 6751 | |
| 543 | 0.1959 | 0.000052 | 264.8 | |
| 545 | 0.03 | 0.0000311 | 10400 | |

### Example D: Prophetic Formulations

“Active ingredient” (a.i.) as used throughout these examples relates to a compound of Formula (I), including any tautomer or stereoisomeric form thereof, or a pharmaceutically acceptable addition salt, or a solvate thereof, in particular to any one of the exemplified compounds.

Typical examples of recipes for the formulation of the invention are as follows:

### 1. Tablets

| Active ingredient | 5 to 50 | mg |
|---|---|---|
| Di-calcium phosphate | 20 | mg |
| Lactose | 30 | mg |
| Talcum | 10 | mg |
| Magnesium stearate | 5 | mg |
| Potato starch | ad 200 | mg |

### 2. Suspension

An aqueous suspension is prepared for oral administration so that each milliliter contains 1 to 5 mg of active ingredient, 50 mg of sodium carboxymethyl cellulose, 1 mg of sodium benzoate, 500 mg of sorbitol and water ad 1 ml.

### 3. Injectable

A parenteral composition is prepared by stirring 1.5% (weight/volume) of active ingredient in 0.9% NaCl solution or in 10% by volume propylene glycol in water.

### 4. Ointment

| Active ingredient | 5 to 1000 | mg |
|---|---|---|
| Stearyl alcohol | 3 | g |
| Lanoline | 5 | g |
| White petroleum | 15 | g |
| Water | ad 100 | g |

In this Example, active ingredient can be replaced with the same amount of any of the compounds according to the present invention, in particular by the same amount of any of the exemplified compounds.

1. A compound of formula (I), including any tautomeric and stereochemically isomeric form, isotopically labeled derivative, or a pharmaceutically acceptable salt or solvate thereof: 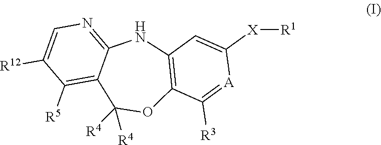
wherein,
X is a 5-6 membered non-aromatic heterocycle; —NH—C(O)—; —NH—CH₂—; —CH₂—; —CH₂—CH₂—; —CH—CH—; absent; a pyridine; a pyrimidine; a 4-7 membered non-aromatic heterocycle; a 4-10 membered non-aromatic bridged heterocycle; C₃₋₇cycloalkyl; or C₅₋₇cycloalkenyl; wherein each of the cycles, independently, may be optionally substituted with —C₁₋₃alkyl, halo, or hydroxy;
R¹ is a 4-5 membered non-aromatic heteromonocycle or a 4-9 membered non-aromatic heteromonocycle, heterobicycle, or spiro-heterobicycle having at least one nitrogen atom, wherein the at least one nitrogen atom is substituted with —C(═O)—CH═CH—R⁶, or —C(═O)—CH═CH—R⁷, and wherein the 4-5 or 4-9 membered non-aromatic heterocycle is optionally substituted with C₁₋₃alkyl, halo, or D; or R¹ is phenyl or pyridine, each independently, substituted with —NR¹¹—C(═O)—CH═CH—R⁶, or —NR¹¹—C(═O)—CH═CH—R⁷, and said phenyl or pyridine is optionally substituted with C₂₋₅alkenyl, C₂₋₅alkynyl, or —O—C₂₋₅alkenyl; or R¹ is C₁₋₃alkyl substituted with —NH—C(═O)—CH═CH—R⁶ or —NH—C(═O)—CH═CH—R⁷;
A is a CR² or N;
R² is H, C₁₋₃alkyl, cyano, halo, or C₂₋₃alkynyl;
R³ is C₁₋₃alkyl, H, halogen, C₂₋₃alkenyl, C₂₋₃alkynyl, cyano, C₃₋₇cycloalkyl; C₁₋₃alkyl substituted with one, two, or three halo, hydroxy, carboxyl, amino, mono- or di(C₁₋₆alkyl)amino; or 1-imidazolyl, 2-imidazolyl, 4-imidazolyl;
R⁴ is, each independently, hydrogen; methyl; C₁₋₃alkyl; C₁₋₃alkyl substituted with one, two, or three halo;
R⁵ is 4-morpholinyl, 4-tetrahydropyranyl, 4-pyrazolyl, a 4-7 membered saturated or partially unsaturated heterocycle, a 5-6 membered heteroaryl, or a 6-12 membered spiro-bicyclic heterocycle; wherein each of the cycles have one, two, or three heteroatoms selected from sulphur, nitrogen, and oxygen; and wherein,
said sulphur, if present, is substituted with dioxo, or with oxo and imino;
said one, two, or three nitrogens, if present, may, each independently, be optionally substituted with C₁₋₃alkyl;
any one of the carbon atoms of the cycles may be optionally substituted with C₁₋₃alkyl, hydroxyC₁₋₃alkyl, C₁₋₃alkoxy, oxo, C₁₋₃alkylsulfonyl, cyano, hydroxy, halo, carboxyl, mono- or di(C₁₋₆alkyl)amino, polyhaloC₁₋₃alkyl, polyhaloC₁₋₃alkoxy, C₂₋₃alkenyl, and C₂₋₃alkynyl;
R⁶ is H; —C₁₋₃alkyl optionally substituted with one, two, or three substituents selected from halo, D, 4-morpholinyl, and —NR^{7a}R^{7b}; wherein each of R^{7a} and R^{7b} is, independently, C₁₋₃alkyl, C₂₋₄alkenyl, or C₂₋₄alkynyl; or R^{7a} and R^{7b} taken together form a heterocycle;
R⁷ is —C₁₋₃alkyl optionally substituted with one, two, or three substituents selected from halo, D, and —NR^{7a}R^{7b}; wherein each of R^{7a} and R^{7b} is, independently, C₁₋₃alkyl, C₂₋₄alkyl, or C₂₋₄alkyl; or R^{7a} and R^{7b} taken together form a heterocycle;
R¹¹ is C₂₋₅alkenyl or C₂₋₅alkynyl; and
R¹² is hydrogen, halo, methyl, or cyano.

2. The compound according to claim 1, including any tautomeric and stereochemically isomeric form, isotopically labeled derivative, or a pharmaceutically acceptable salt or solvate thereof,
wherein,
X is a 5-6 membered non-aromatic heterocycle optionally substituted with —C₁₋₃alkyl;
R¹ is a 4-5 membered non-aromatic heterocycle having at least one nitrogen atom, wherein the at least one nitrogen atom is substituted with —C(═O)—CH═CH—R⁶, or —C(═O)—CH—CH—R⁷, and wherein the 4-5 membered non-aromatic heterocycle is optionally substituted with C₁₋₃alkyl, halo, or D;
A is a CR² or N;
R² is H, C₁₋₃alkyl, or cyano;
R³ is C₁₋₃alkyl, H, halogen, cyano, C₃₋₇cycloalkyl; or C₁₋₃alkyl substituted with one, two, or three halo;
R⁴ is, each independently, hydrogen or methyl;
R⁵ a 4-morpholinyl, 4-tetrahydropyranyl, or 4-pyrazolyl;
R⁶ is H; —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle;
R⁷ is —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle; and
R¹² is hydrogen.

3. The compound according to claim 1, wherein the compound is of formula (II), including any tautomeric and stereochemically isomeric form, isotopically labeled derivative, or a pharmaceutically acceptable salt or solvate thereof,

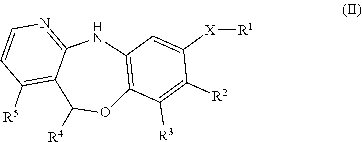

4. The compound according to claim 1, wherein the compound is of formula (IIa), (IIb), (IIc), (IId), (IIe), or (IIf), including any tautomeric and stereochemically isomeric form, isotopically labeled derivative, or a pharmaceutically acceptable salt or solvate thereof: 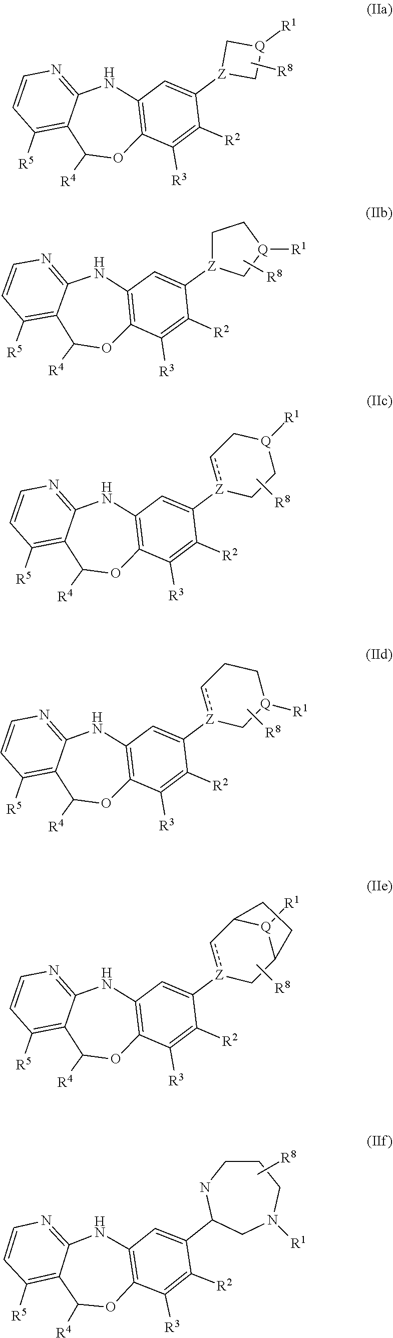
wherein in each of the compounds of formula (IIa), (IIb), (IIc), (IId), (IIe), or (IIf),
each Q is, independently, CH or N;
each Z is, independently, CH or N;
each R⁸ is, independently, H or —C₁₋₃alkyl; and said R⁸ may be bound to any carbon or nitrogen atom of the cycle; and
each dashed bond is, independently, an optional double bond.

5. The compound according to claim 1, wherein the compound is of formula (IIIa), (IIIb), (IIIc), (IIId), (IIIe), or (IIIf), including any tautomeric and stereochemically isomeric form, isotopically labeled derivative, or a pharmaceutically acceptable salt or solvate thereof: 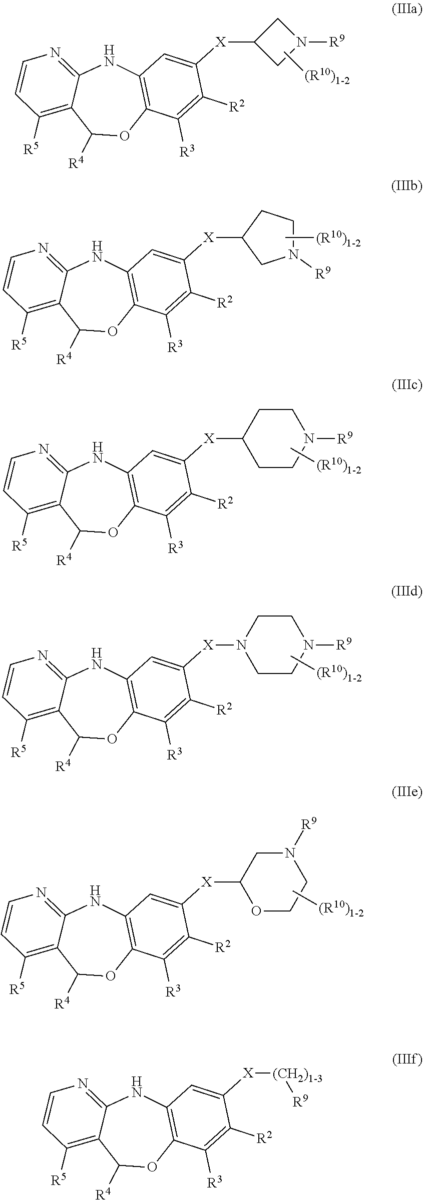
wherein
each R⁹ is, independently, —C(═O)—CH═CH—R⁶, or —C(═O)—CH—CH—R⁷;
each R¹⁰ is, independently, H, —C₁₋₃alkyl, halo, or D; and said R¹⁰ may be bound to any carbon atom of the cycle; and.

6. The compound according to claim 1, wherein the compound is of formula (IVa), (IVb), (IVc), (IVd), (IVe), (IVf), (IVg), (IVh), (IVi), (IVj), (IVk), (IVl), (IVm), (IVn), (Ivo), (IVp), or (IVq), including any tautomeric and stereochemically isomeric form, isotopically labeled derivative, or a pharmaceutically acceptable salt or solvate thereof:

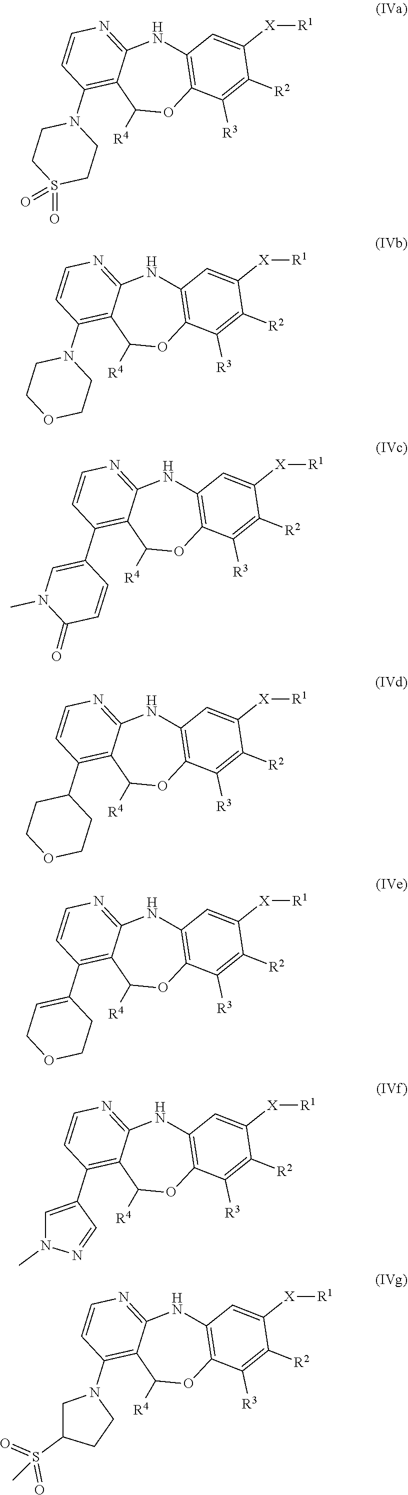

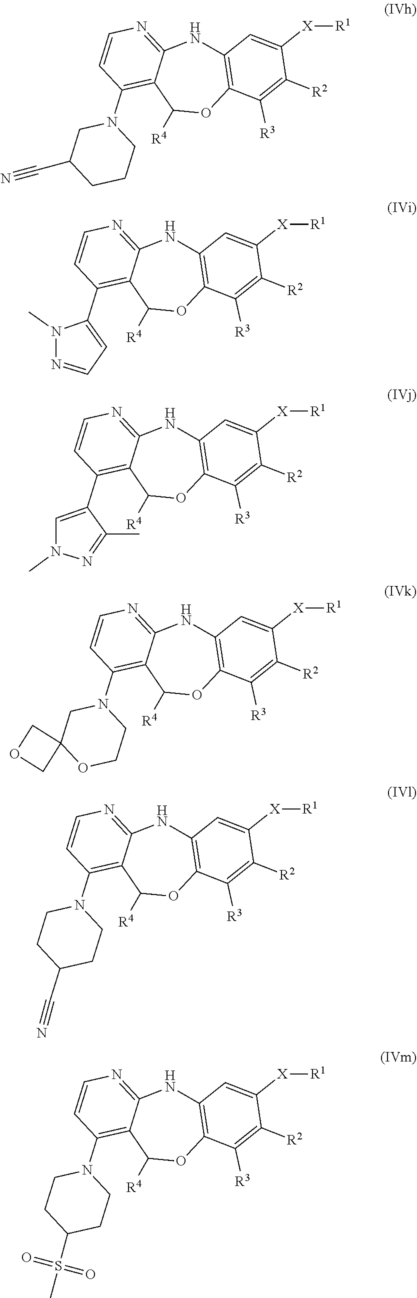

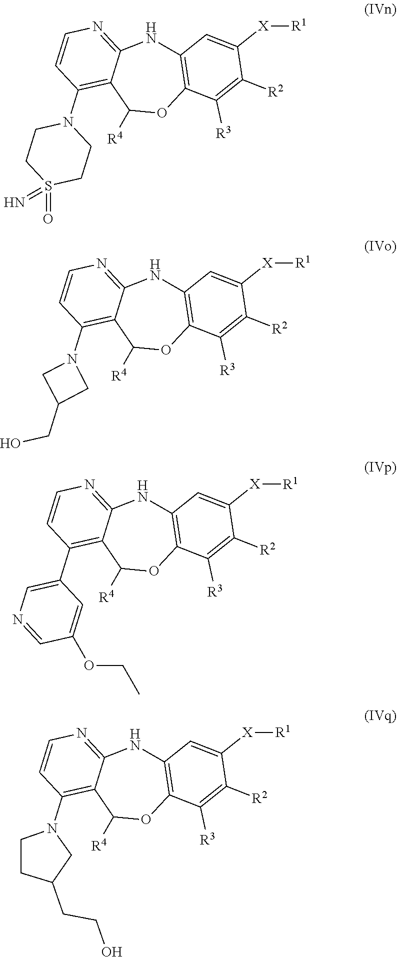

7. The compound according to claim 1, wherein the compound is of formula (Va) or (Vb), including any tautomeric and stereochemically isomeric form, isotopically labeled derivative, or a pharmaceutically acceptable salt or solvate thereof.

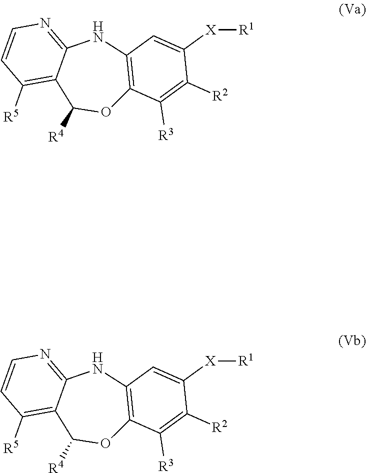

8. The compound according to claim 1, wherein the compound is of formula (VI), including any tautomeric and stereochemically isomeric form, isotopically labeled derivative, or a pharmaceutically acceptable salt or solvate thereof,

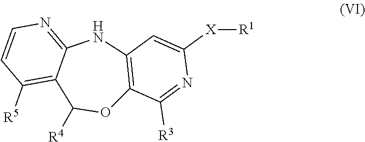

9. The compound according to claim 8, including any tautomeric and stereochemically isomeric form, isotopically labeled derivative, or a pharmaceutically acceptable salt or solvate thereof,
wherein,
X is a 4-7 membered non-aromatic heterocycle;
R¹ is a 4-7 membered non-aromatic heterocycle having at least one nitrogen atom, wherein the at least one nitrogen atom is substituted with —C(═O)—CH═CH—R⁶, or —C(═O)—CH—CH—R⁷;
R³ is C₁₋₃alkyl, H, halogen, cyano, C₃₋₇cycloalkyl; or C₁₋₃alkyl substituted with one, two, or three halo;
R⁴ is methyl or H;
R⁵ is a 4-7 membered saturated or partially unsaturated heterocycle, a 5-6 membered heteroaryl, or a 6-12 membered spiro-bicyclic heterocycle; wherein each of the cycles have one, two, or three heteroatoms selected from sulphur, nitrogen, and oxygen; and wherein,
said sulphur, if present, is substituted with dioxo, or with oxo and imino;
said one, two, or three nitrogens, if present, may, each independently, be optionally substituted with C₁₋₃alkyl;
any one of the carbon atoms of the cycles may be optionally substituted with C₁₋₃alkyl, hydroxyC₁₋₃alkyl, C₁₋₃alkoxy, oxo, C₁₋₃alkylsulfonyl, cyano, hydroxy, halo, carboxyl, mono- or di(C₁₋₆alkyl)amino, polyhaloC₁₋₃alkyl, polyhaloC₁₋₃alkoxy, C₂₋₃alkenyl, and C₂₋₃alkynyl;
R⁶ is H; —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle; and
R⁷ is —C₁₋₃alkyl optionally substituted with one, two, or three substituents selected from halo, D, and —NR^{7a}R^{7b}; wherein each of R^{7a} and R^{7b} is, independently, C₁₋₃alkyl; or R^{7a} and R^{7b} taken together form a heterocycle.

10. The compound according to claim 8, wherein the compound is of formula (VIIa), (VIIb), (VIIc), (VIId), (VIIe), or (VIIf), including any tautomeric and stereochemically isomeric form, isotopically labeled derivative, or a pharmaceutically acceptable salt or solvate thereof: 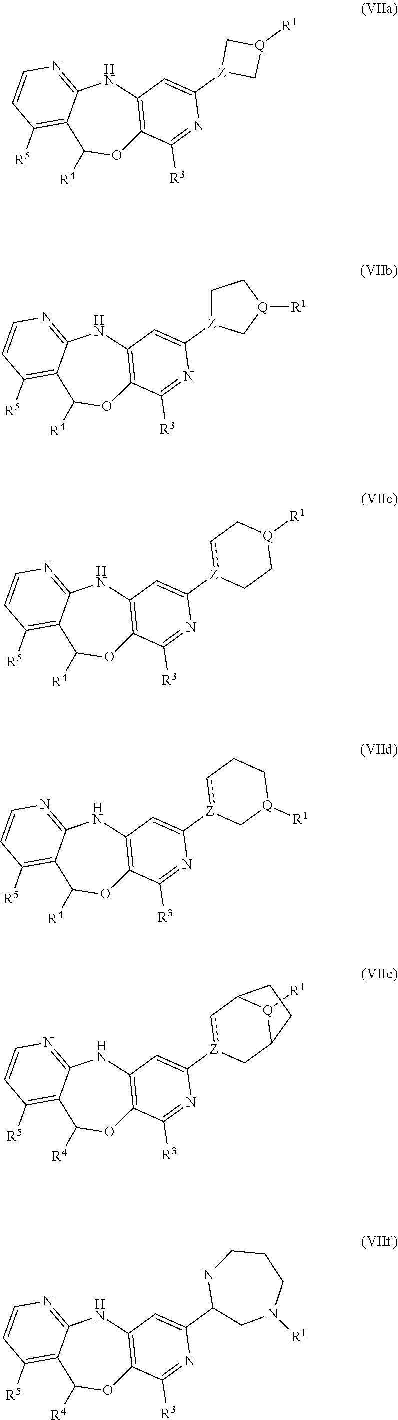
wherein,
each Q is, independently, CH or N;
each Z is, independently, CH or N.

11. The compound according to claim 8, wherein the compound is of formula (VIIIa), (VIIIb), (VIIIc), (VIIId), (VIIIe), or (VIIIf) including any tautomeric and stereochemically isomeric form, isotopically labeled derivative, or a pharmaceutically acceptable salt or solvate thereof: 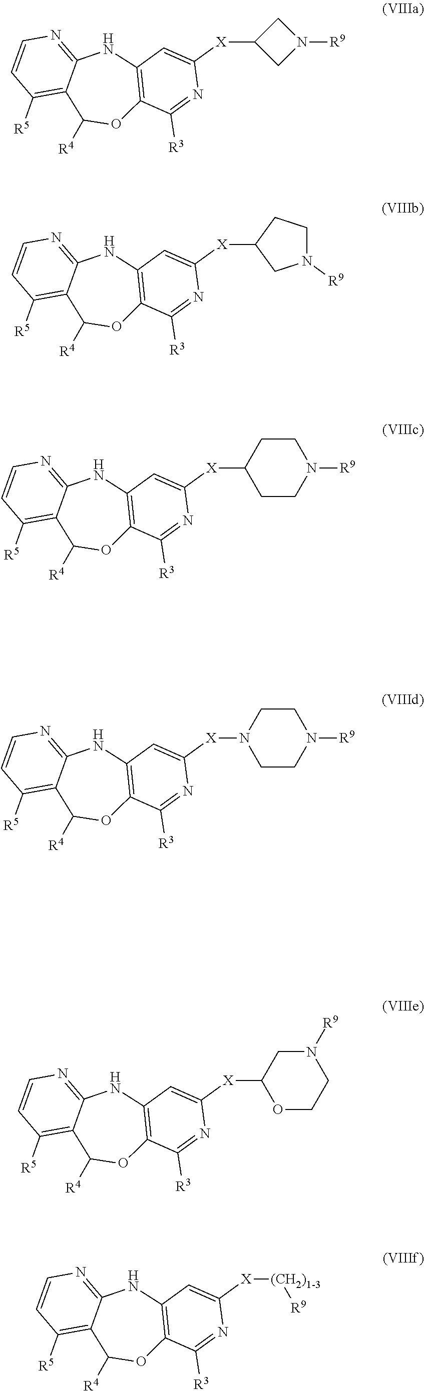
wherein
R⁹ is —C(═O)—CH═CH—R⁶, or —C(═O)—CH—CH—R⁷.

12. The compound according to claim 8, wherein the compound is of formula (IXa), (IXb), (IXc), (IXd), (IXe), (IXf), (IXg), (IXh), (IXi), (IXj), (IXk), (IXl), (IXm), (IXn), (IXo), (IXp), or (IXq), including any tautomeric and stereochemically isomeric form, isotopically labeled derivative, or a pharmaceutically acceptable salt or solvate thereof:

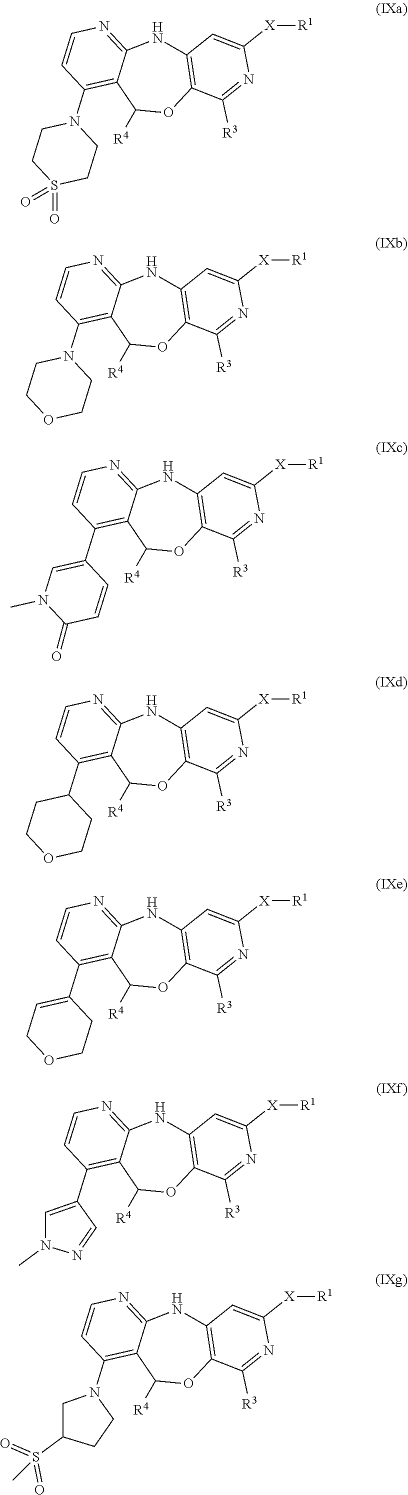

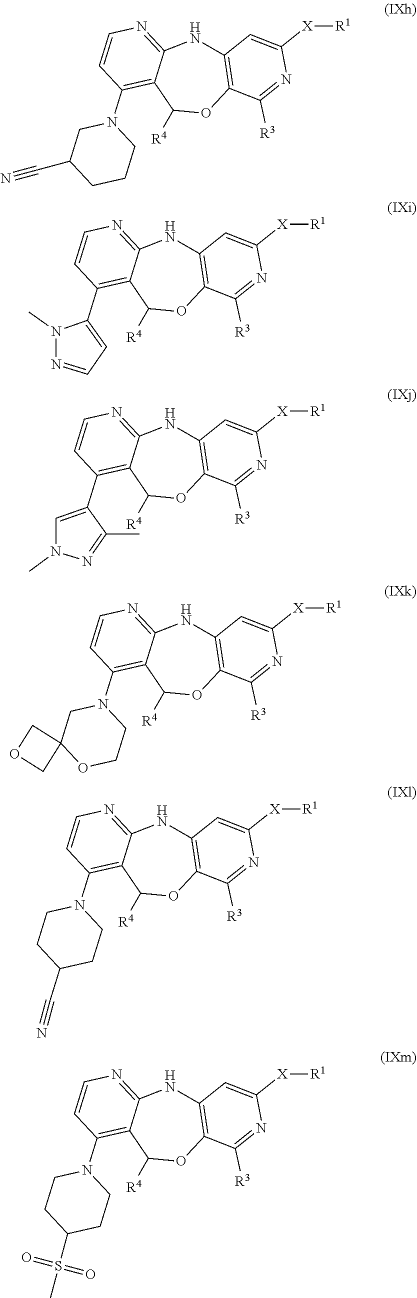

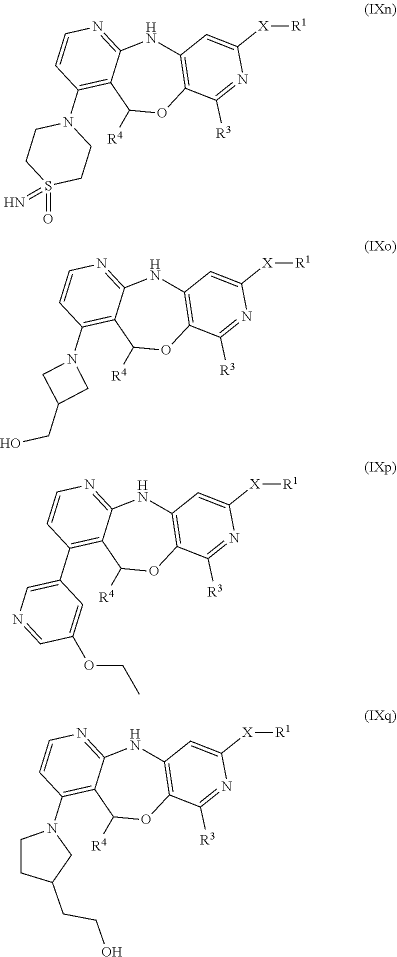

13. The compound according to claim 8, including any tautomeric and stereochemically isomeric form, isotopically labeled derivative, or a pharmaceutically acceptable salt or solvate thereof:
wherein,
R⁵ is

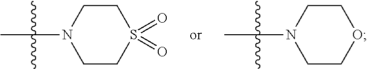 [SMILES: *N1CCS(=O)(=O)CC1 ; *N1CCOCC1]

14. The compound according to claim 8, wherein the compound is of formula (Xa) or (Xb), including any tautomeric and stereochemically isomeric form, isotopically labeled derivative, or a pharmaceutically acceptable salt or solvate thereof:

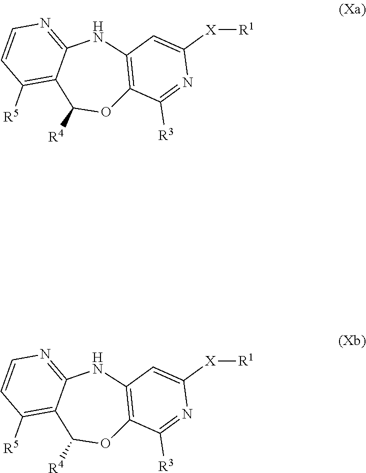

15. The compound according to claim 1, including any tautomeric and stereochemically isomeric form, isotopically labeled derivative, or a pharmaceutically acceptable salt or solvate thereof, wherein the compound is selected from:

| compound | |
|---|---|
| # | STRUCTURE |
| 1 | 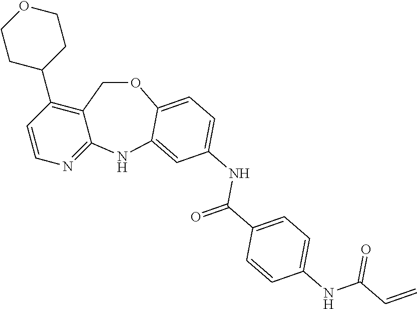 [SMILES: C=CC(=O)Nc1ccc(C(=O)Nc2ccc3c(c2)Nc2nccc(C4CCOCC4)c2CO3)cc1] |
| 2 | 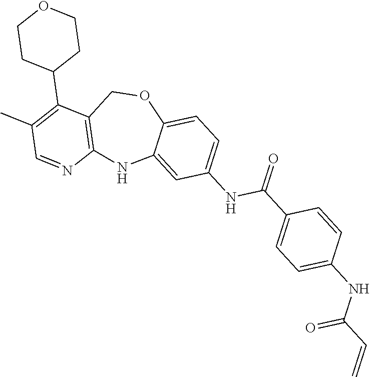 [SMILES: C=CC(=O)Nc1ccc(C(=O)Nc2ccc3c(c2)Nc2ncc(C)c(C4CCOCC4)c2CO3)cc1] |
| 3 | 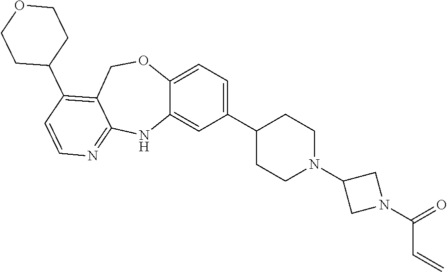 [SMILES: C=CC(=O)N1CC(N2CCC(c3ccc4c(c3)Nc3nccc(C5CCOCC5)c3CO4)CC2)C1] |
| 4 | 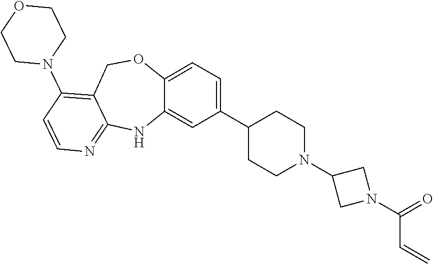 [SMILES: C=CC(=O)N1CC(N2CCC(c3ccc4c(c3)Nc3nccc(N5CCOCC5)c3CO4)CC2)C1] |
| 5 | 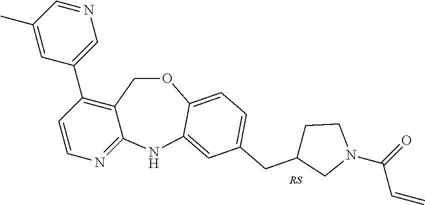 [SMILES: C=CC(=O)N1CCC(Cc2ccc3c(c2)Nc2nccc(-c4cncc(C)c4)c2CO3)C1] |
| 6 | 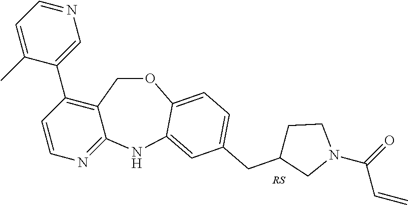 [SMILES: C=CC(=O)N1CCC(Cc2ccc3c(c2)Nc2nccc(-c4cnccc4C)c2CO3)C1] |
| 7 | 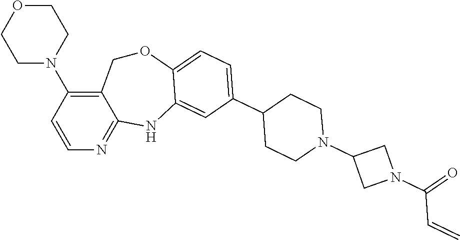 [SMILES: C=CC(=O)N1CC(N2CCC(c3ccc4c(c3)Nc3nccc(N5CCOCC5)c3CO4)CC2)C1] |
| 8 | 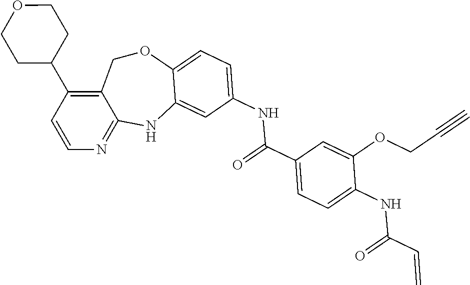 [SMILES: C#CCOc1cc(C(=O)Nc2ccc3c(c2)Nc2nccc(C4CCOCC4)c2CO3)ccc1NC(=O)C=C] |
| 9 | 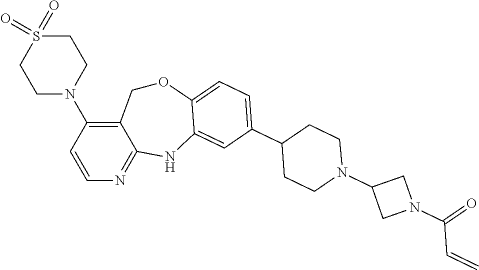 [SMILES: C=CC(=O)N1CC(N2CCC(c3ccc4c(c3)Nc3nccc(N5CCS(=O)(=O)CC5)c3CO4)CC2)C1] |
| 10 | 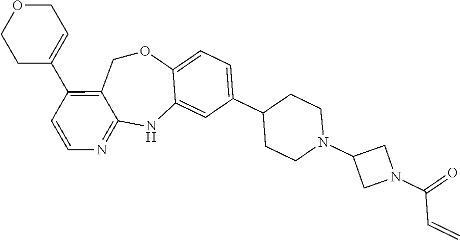 [SMILES: C=CC(=O)N1CC(N2CCC(c3ccc4c(c3)Nc3nccc(C5=CCOCC5)c3CO4)CC2)C1] |
| 11 | 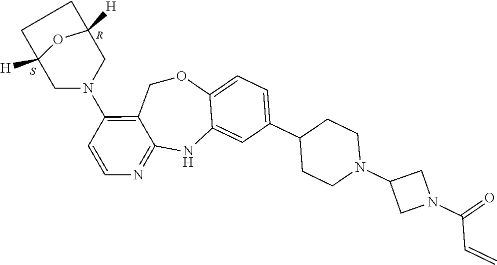 |
| 12 | 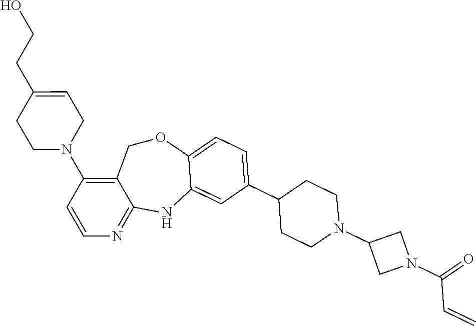 [SMILES: C=CC(=O)N1CC(N2CCC(c3ccc4c(c3)Nc3nccc(N5CC=C(CCO)CC5)c3CO4)CC2)C1] |
| 13 | 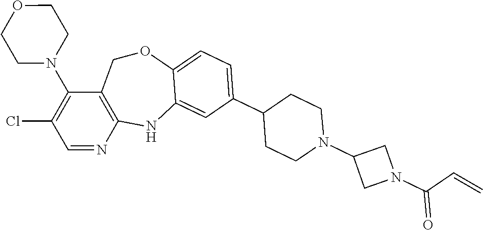 [SMILES: C=CC(=O)N1CC(N2CCC(c3ccc4c(c3)Nc3ncc(Cl)c(N5CCOCC5)c3CO4)CC2)C1] |
| 14 | 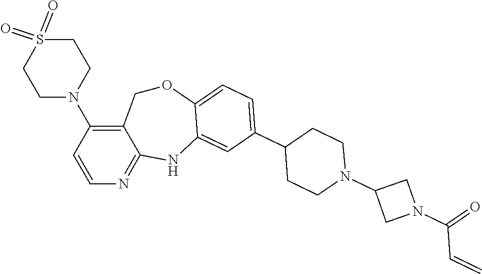 [SMILES: C=CC(=O)N1CC(N2CCC(c3ccc4c(c3)Nc3nccc(N5CCS(=O)(=O)CC5)c3CO4)CC2)C1] |
| 15 | 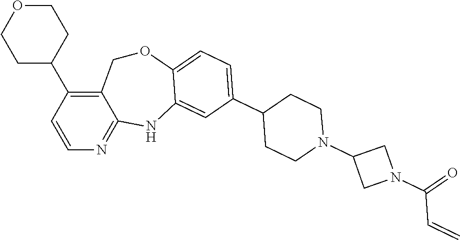 [SMILES: C=CC(=O)N1CC(N2CCC(c3ccc4c(c3)Nc3nccc(C5CCOCC5)c3CO4)CC2)C1] |
| 16 | 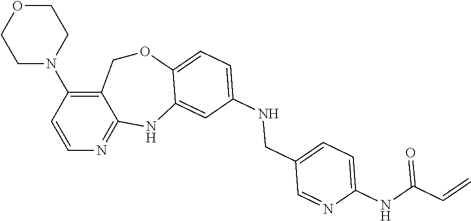 [SMILES: C=CC(=O)Nc1ccc(CNc2ccc3c(c2)Nc2nccc(N4CCOCC4)c2CO3)cn1] |
| 17 | 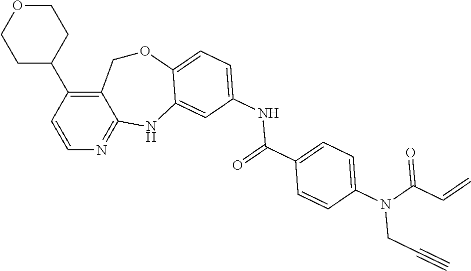 [SMILES: C#CCN(C(=O)C=C)c1ccc(C(=O)Nc2ccc3c(c2)Nc2nccc(C4CCOCC4)c2CO3)cc1] |
| 18 | 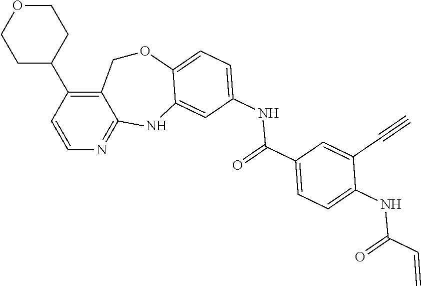 [SMILES: C#Cc1cc(C(=O)Nc2ccc3c(c2)Nc2nccc(C4CCOCC4)c2CO3)ccc1NC(=O)C=C] |
| 19 | 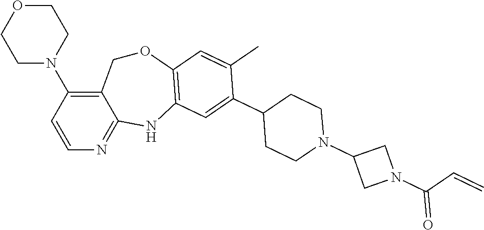 [SMILES: C=CC(=O)N1CC(N2CCC(c3cc4c(cc3C)OCc3c(N5CCOCC5)ccnc3N4)CC2)C1] |
| 20 | 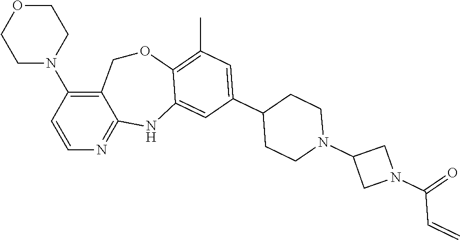 [SMILES: C=CC(=O)N1CC(N2CCC(c3cc(C)c4c(c3)Nc3nccc(N5CCOCC5)c3CO4)CC2)C1] |
| 21 | 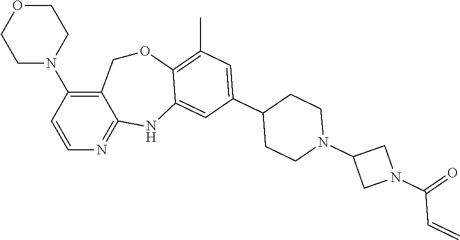 [SMILES: C=CC(=O)N1CC(N2CCC(c3cc(C)c4c(c3)Nc3nccc(N5CCOCC5)c3CO4)CC2)C1] |
| 22 | 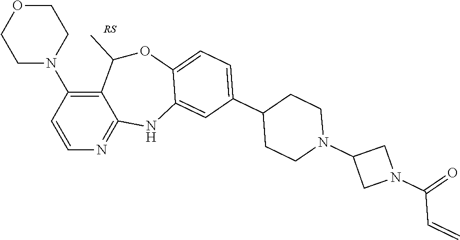 [SMILES: C=CC(=O)N1CC(N2CCC(c3ccc4c(c3)Nc3nccc(N5CCOCC5)c3C(C)O4)CC2)C1] |
| 23 | 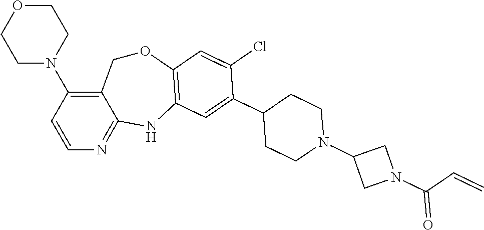 [SMILES: C=CC(=O)N1CC(N2CCC(c3cc4c(cc3Cl)OCc3c(N5CCOCC5)ccnc3N4)CC2)C1] |
| 24 | 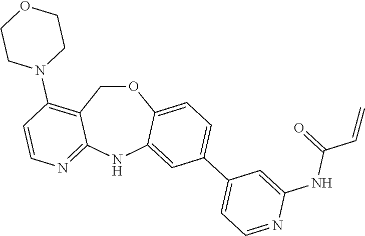 [SMILES: C=CC(=O)Nc1cc(-c2ccc3c(c2)Nc2nccc(N4CCOCC4)c2CO3)ccn1] |
| 25 | 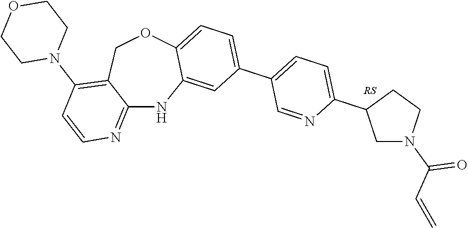 [SMILES: C=CC(=O)N1CCC(c2ccc(-c3ccc4c(c3)Nc3nccc(N5CCOCC5)c3CO4)cn2)C1] |
| 26 | 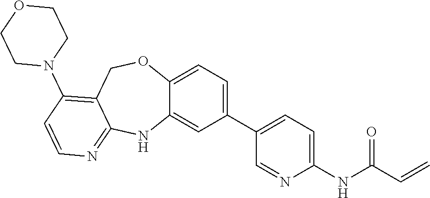 [SMILES: C=CC(=O)Nc1ccc(-c2ccc3c(c2)Nc2nccc(N4CCOCC4)c2CO3)cn1] |
| 27 | 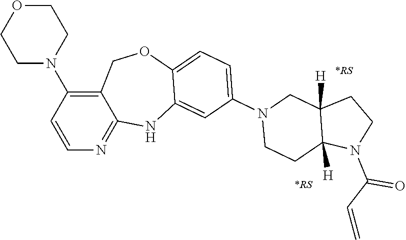 [SMILES: C=CC(=O)N1CC[C@H]2CN(c3ccc4c(c3)Nc3nccc(N5CCOCC5)c3CO4)CC[C@H]21] |
| 28 | 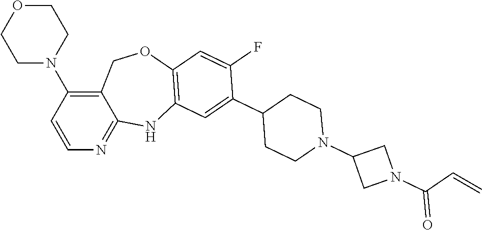 [SMILES: C=CC(=O)N1CC(N2CCC(c3cc4c(cc3F)OCc3c(N5CCOCC5)ccnc3N4)CC2)C1] |
| 29 | 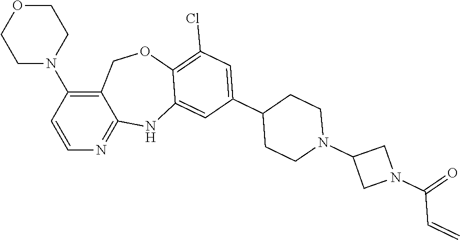 [SMILES: C=CC(=O)N1CC(N2CCC(c3cc(Cl)c4c(c3)Nc3nccc(N5CCOCC5)c3CO4)CC2)C1] |
| 30 | 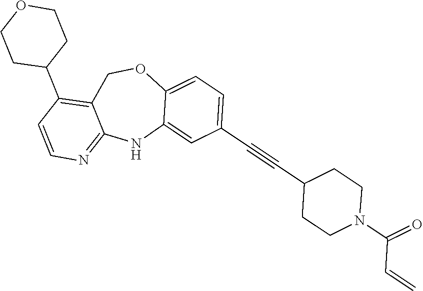 [SMILES: C=CC(=O)N1CCC(C#Cc2ccc3c(c2)Nc2nccc(C4CCOCC4)c2CO3)CC1] |
| 31 | 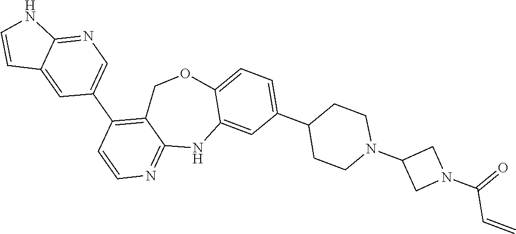 [SMILES: C=CC(=O)N1CC(N2CCC(c3ccc4c(c3)Nc3nccc(-c5cnc6[nH]ccc6c5)c3CO4)CC2)C1] |
| 32 | 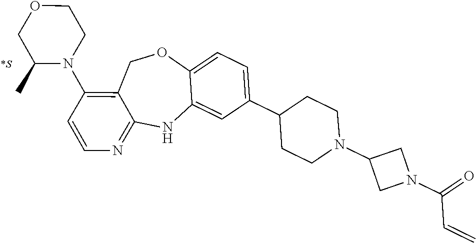 [SMILES: C=CC(=O)N1CC(N2CCC(c3ccc4c(c3)Nc3nccc(N5CCOC[C@@H]5C)c3CO4)CC2)C1] |
| 33 | 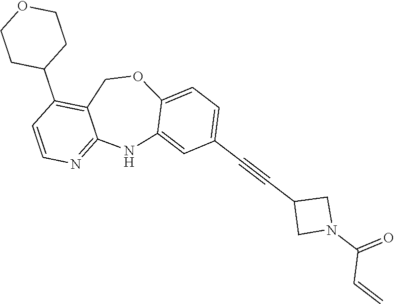 [SMILES: C=CC(=O)N1CC(C#Cc2ccc3c(c2)Nc2nccc(C4CCOCC4)c2CO3)C1] |
| 34 | 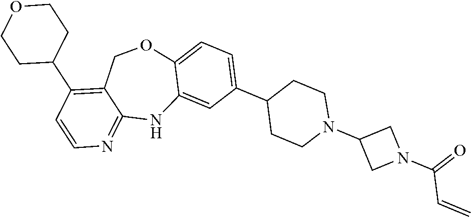 [SMILES: C=CC(=O)N1CC(N2CCC(c3ccc4c(c3)Nc3nccc(C5CCOCC5)c3CO4)CC2)C1] |
| 35 | 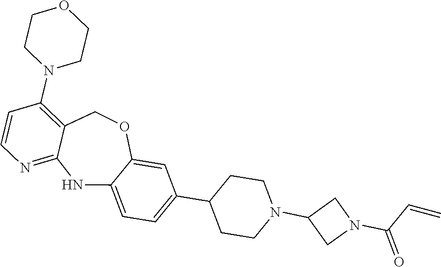 [SMILES: C=CC(=O)N1CC(N2CCC(c3ccc4c(c3)OCc3c(N5CCOCC5)ccnc3N4)CC2)C1] |
| 36 | 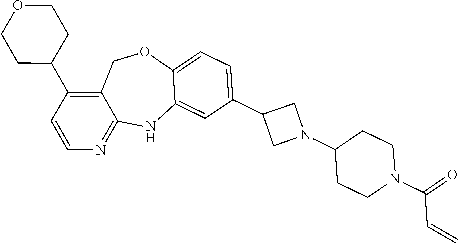 [SMILES: C=CC(=O)N1CCC(N2CC(c3ccc4c(c3)Nc3nccc(C5CCOCC5)c3CO4)C2)CC1] |
| 37 | 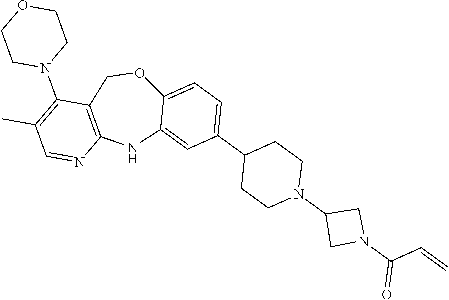 [SMILES: C=CC(=O)N1CC(N2CCC(c3ccc4c(c3)Nc3ncc(C)c(N5CCOCC5)c3CO4)CC2)C1] |
| 38 | 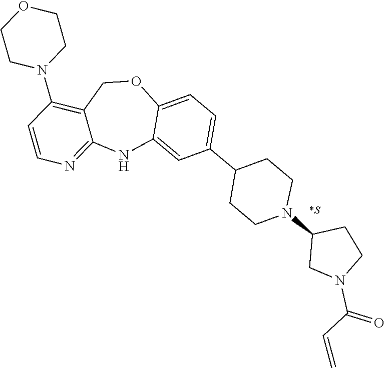 [SMILES: C=CC(=O)N1CC[C@H](N2CCC(c3ccc4c(c3)Nc3nccc(N5CCOCC5)c3CO4)CC2)C1] |
| 39 | 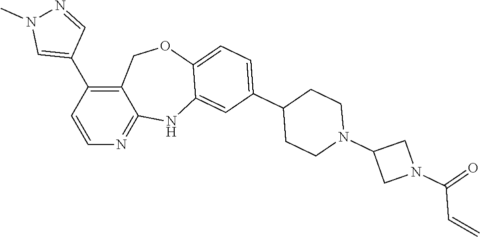 [SMILES: C=CC(=O)N1CC(N2CCC(c3ccc4c(c3)Nc3nccc(-c5cnn(C)c5)c3CO4)CC2)C1] |
| 40 | 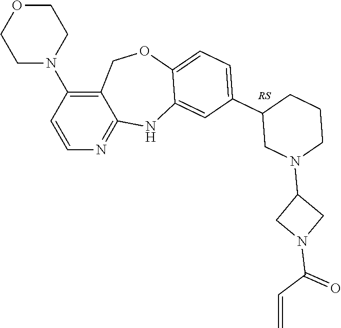 [SMILES: C=CC(=O)N1CC(N2CCCC(c3ccc4c(c3)Nc3nccc(N5CCOCC5)c3CO4)C2)C1] |
| 41 | 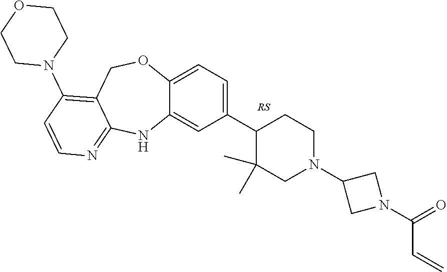 [SMILES: C=CC(=O)N1CC(N2CCC(c3ccc4c(c3)Nc3nccc(N5CCOCC5)c3CO4)C(C)(C)C2)C1] |
| 42 | 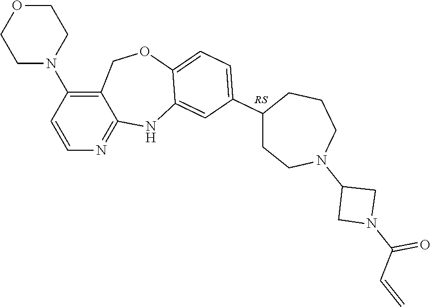 [SMILES: C=CC(=O)N1CC(N2CCCC(c3ccc4c(c3)Nc3nccc(N5CCOCC5)c3CO4)CC2)C1] |
| 43 | 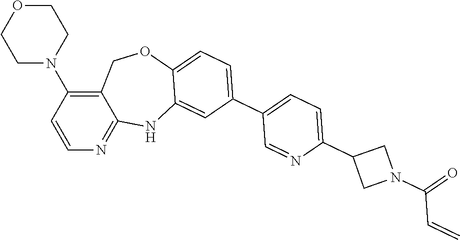 [SMILES: C=CC(=O)N1CC(c2ccc(-c3ccc4c(c3)Nc3nccc(N5CCOCC5)c3CO4)cn2)C1] |
| 44 | 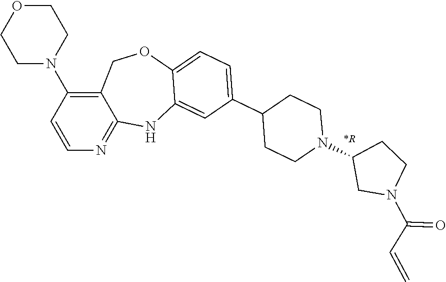 [SMILES: C=CC(=O)N1CC[C@@H](N2CCC(c3ccc4c(c3)Nc3nccc(N5CCOCC5)c3CO4)CC2)C1] |
| 45 | 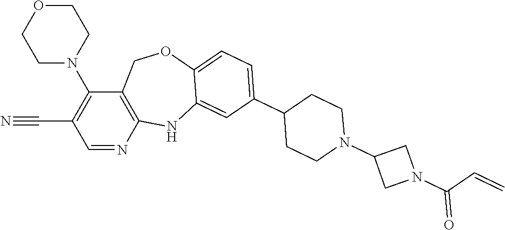 [SMILES: C=CC(=O)N1CC(N2CCC(c3ccc4c(c3)Nc3ncc(C#N)c(N5CCOCC5)c3CO4)CC2)C1] |
| 46 | 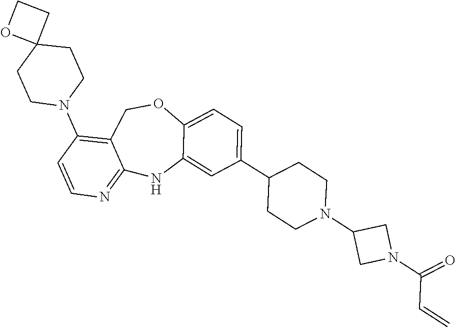 [SMILES: C=CC(=O)N1CC(N2CCC(c3ccc4c(c3)Nc3nccc(N5CCC6(CCO6)CC5)c3CO4)CC2)C1] |
| 47 | 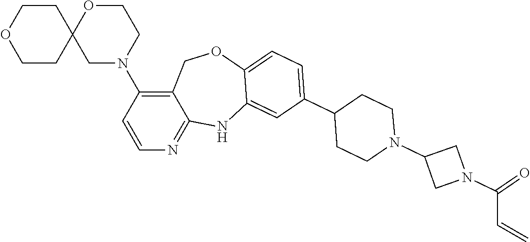 [SMILES: C=CC(=O)N1CC(N2CCC(c3ccc4c(c3)Nc3nccc(N5CCOC6(CCOCC6)C5)c3CO4)CC2)C1] |
| 48 | 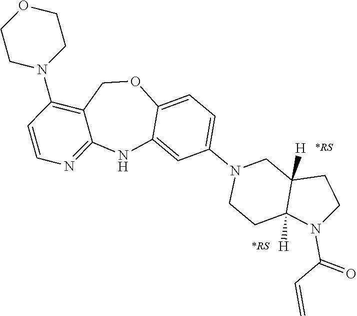 [SMILES: C=CC(=O)N1CC[C@H]2CN(c3ccc4c(c3)Nc3nccc(N5CCOCC5)c3CO4)CC[C@@H]21] |
| 49 | 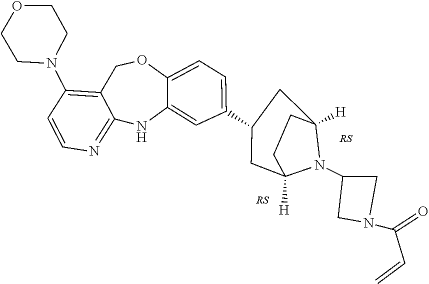 [SMILES: C=CC(=O)N1CC(N2[C@@H]3CC[C@H]2C[C@@H](c2ccc4c(c2)Nc2nccc(N5CCOCC5)c2CO4)C3)C1] |
| 50 | 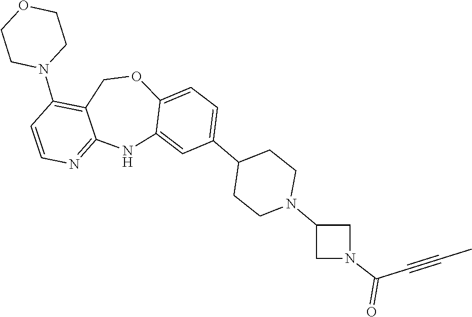 [SMILES: CC#CC(=O)N1CC(N2CCC(c3ccc4c(c3)Nc3nccc(N5CCOCC5)c3CO4)CC2)C1] |
| 51 | 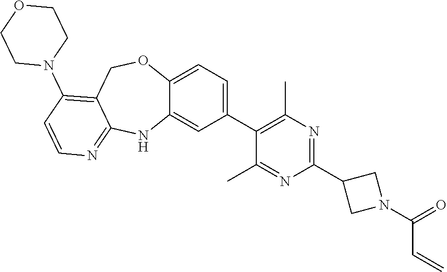 [SMILES: C=CC(=O)N1CC(c2nc(C)c(-c3ccc4c(c3)Nc3nccc(N5CCOCC5)c3CO4)c(C)n2)C1] |
| 52 | 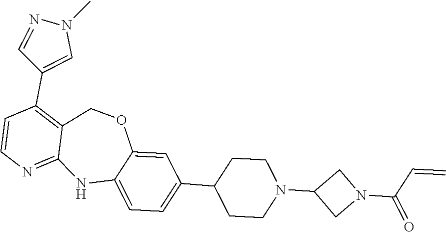 [SMILES: C=CC(=O)N1CC(N2CCC(c3ccc4c(c3)OCc3c(-c5cnn(C)c5)ccnc3N4)CC2)C1] |
| 53 | 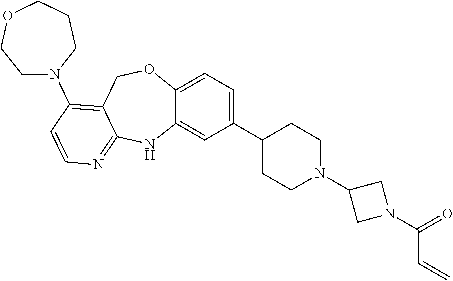 [SMILES: C=CC(=O)N1CC(N2CCC(c3ccc4c(c3)Nc3nccc(N5CCCOCC5)c3CO4)CC2)C1] |
| 54 | 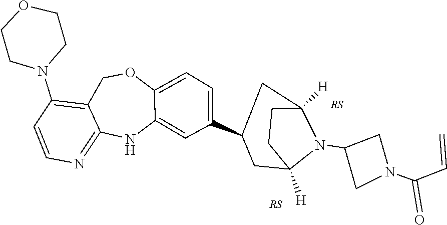 [SMILES: C=CC(=O)N1CC(N2[C@@H]3CC[C@H]2C[C@H](c2ccc4c(c2)Nc2nccc(N5CCOCC5)c2CO4)C3)C1] |
| 55 | 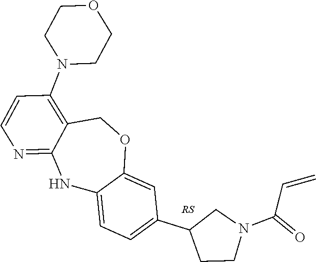 [SMILES: C=CC(=O)N1CCC(c2ccc3c(c2)OCc2c(N4CCOCC4)ccnc2N3)C1] |
| 56 | 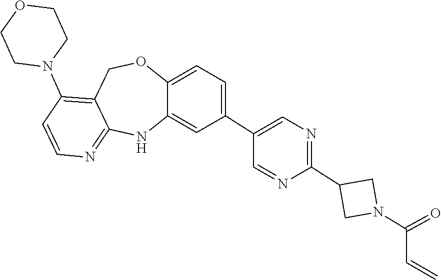 [SMILES: C=CC(=O)N1CC(c2ncc(-c3ccc4c(c3)Nc3nccc(N5CCOCC5)c3CO4)cn2)C1] |
| 57 | 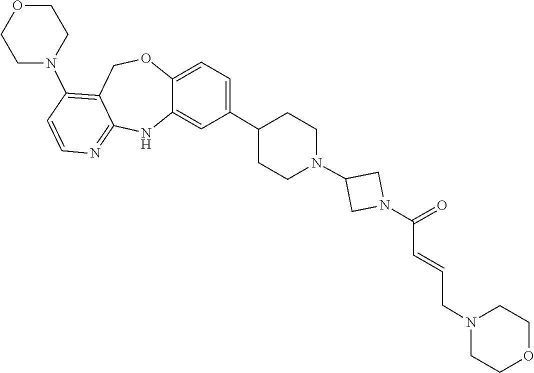 [SMILES: O=C(/C=C/CN1CCOCC1)N1CC(N2CCC(c3ccc4c(c3)Nc3nccc(N5CCOCC5)c3CO4)CC2)C1] |
| 58 | 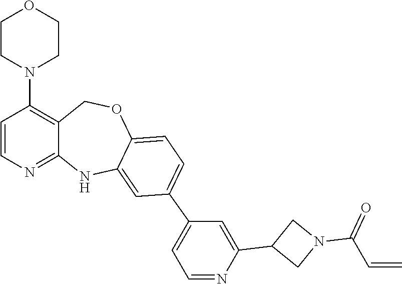 [SMILES: C=CC(=O)N1CC(c2cc(-c3ccc4c(c3)Nc3nccc(N5CCOCC5)c3CO4)ccn2)C1] |
| 59 | 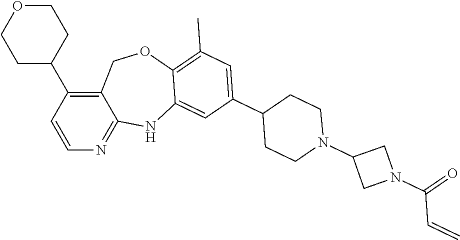 [SMILES: C=CC(=O)N1CC(N2CCC(c3cc(C)c4c(c3)Nc3nccc(C5CCOCC5)c3CO4)CC2)C1] |
| 60 | 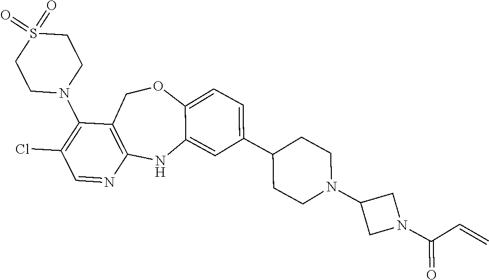 [SMILES: C=CC(=O)N1CC(N2CCC(c3ccc4c(c3)Nc3ncc(Cl)c(N5CCS(=O)(=O)CC5)c3CO4)CC2)C1] |
| 61 | 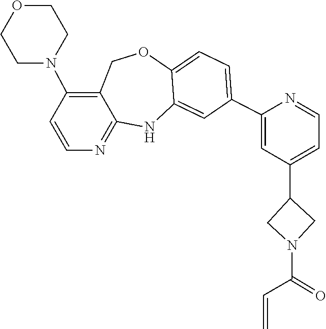 [SMILES: C=CC(=O)N1CC(c2ccnc(-c3ccc4c(c3)Nc3nccc(N5CCOCC5)c3CO4)c2)C1] |
| 62 | 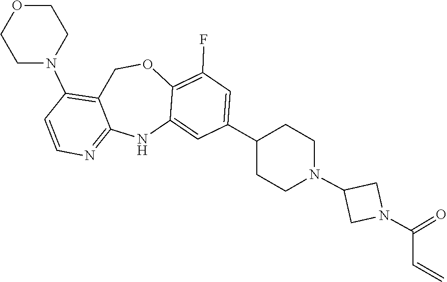 [SMILES: C=CC(=O)N1CC(N2CCC(c3cc(F)c4c(c3)Nc3nccc(N5CCOCC5)c3CO4)CC2)C1] |
| 63 | 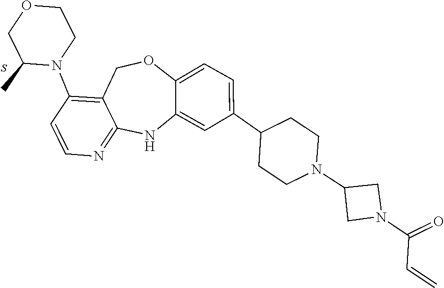 |
| 64 | 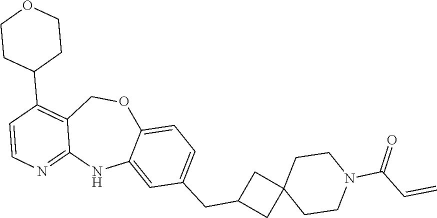 [SMILES: C=CC(=O)N1CCC2(CC1)CC(Cc1ccc3c(c1)Nc1nccc(C4CCOCC4)c1CO3)C2] |
| 65 | 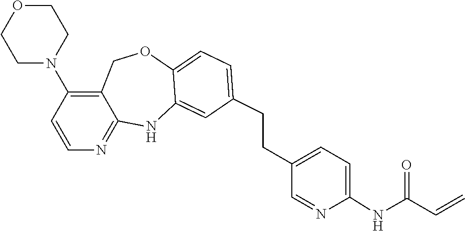 [SMILES: C=CC(=O)Nc1ccc(CCc2ccc3c(c2)Nc2nccc(N4CCOCC4)c2CO3)cn1] |
| 66 | 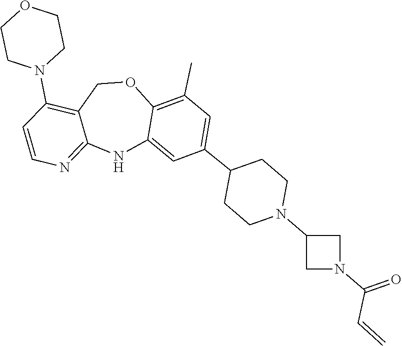 [SMILES: C=CC(=O)N1CC(N2CCC(c3cc(C)c4c(c3)Nc3nccc(N5CCOCC5)c3CO4)CC2)C1] |
| 67 | 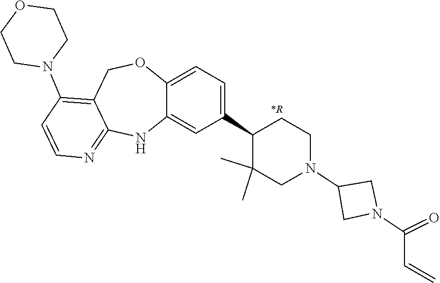 [SMILES: C=CC(=O)N1CC(N2CC[C@H](c3ccc4c(c3)Nc3nccc(N5CCOCC5)c3CO4)C(C)(C)C2)C1] |
| 68 | 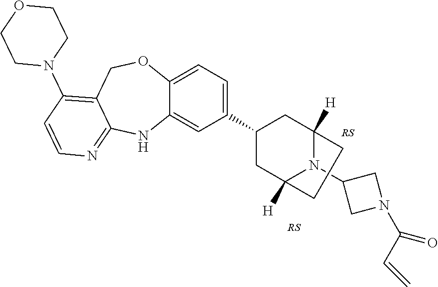 [SMILES: C=CC(=O)N1CC(N2[C@@H]3CC[C@H]2C[C@@H](c2ccc4c(c2)Nc2nccc(N5CCOCC5)c2CO4)C3)C1] |
| 69 | 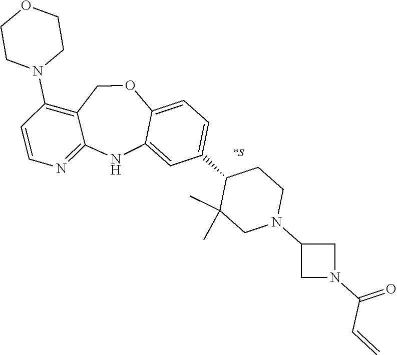 [SMILES: C=CC(=O)N1CC(N2CC[C@@H](c3ccc4c(c3)Nc3nccc(N5CCOCC5)c3CO4)C(C)(C)C2)C1] |
| 70 | 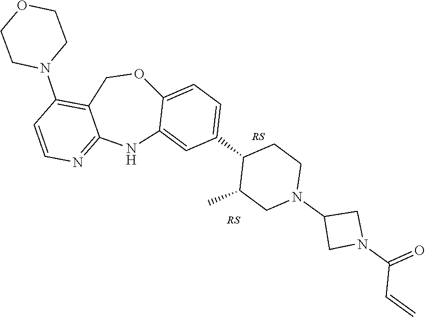 [SMILES: C=CC(=O)N1CC(N2CC[C@@H](c3ccc4c(c3)Nc3nccc(N5CCOCC5)c3CO4)[C@@H](C)C2)C1] |
| 71 | 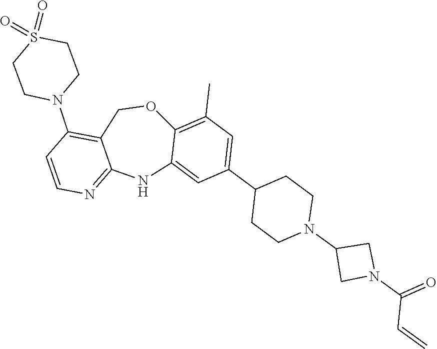 [SMILES: C=CC(=O)N1CC(N2CCC(c3cc(C)c4c(c3)Nc3nccc(N5CCS(=O)(=O)CC5)c3CO4)CC2)C1] |
| 72 | 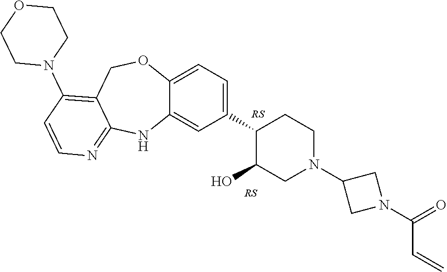 [SMILES: C=CC(=O)N1CC(N2CC[C@@H](c3ccc4c(c3)Nc3nccc(N5CCOCC5)c3CO4)[C@H](O)C2)C1] |
| 73 | 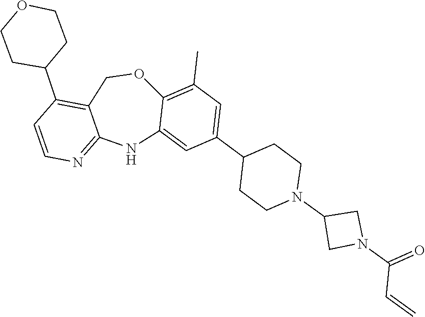 [SMILES: C=CC(=O)N1CC(N2CCC(c3cc(C)c4c(c3)Nc3nccc(C5CCOCC5)c3CO4)CC2)C1] |
| 74 | 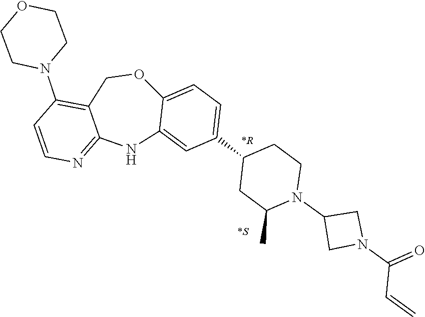 [SMILES: C=CC(=O)N1CC(N2CC[C@@H](c3ccc4c(c3)Nc3nccc(N5CCOCC5)c3CO4)C[C@@H]2C)C1] |
| 75 | 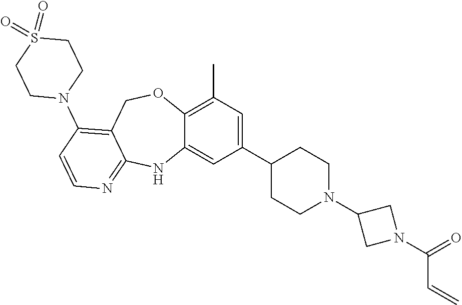 [SMILES: C=CC(=O)N1CC(N2CCC(c3cc(C)c4c(c3)Nc3nccc(N5CCS(=O)(=O)CC5)c3CO4)CC2)C1] |
| 76 | 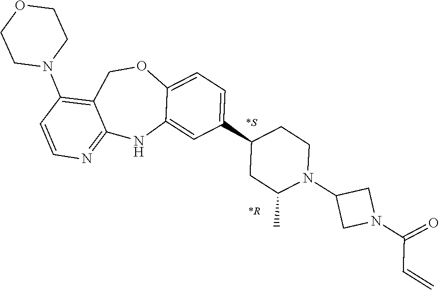 [SMILES: C=CC(=O)N1CC(N2CC[C@H](c3ccc4c(c3)Nc3nccc(N5CCOCC5)c3CO4)C[C@H]2C)C1] |
| 77 | 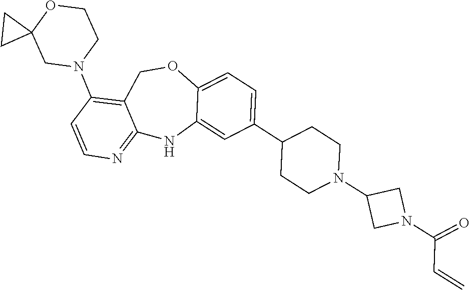 [SMILES: C=CC(=O)N1CC(N2CCC(c3ccc4c(c3)Nc3nccc(N5CCOC6(CC6)C5)c3CO4)CC2)C1] |
| 78 | 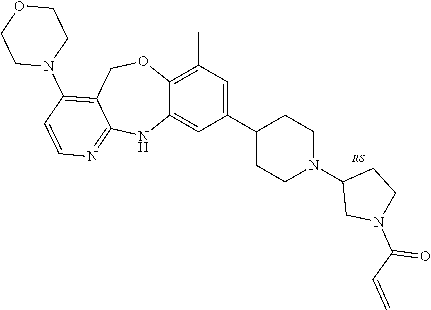 [SMILES: C=CC(=O)N1CCC(N2CCC(c3cc(C)c4c(c3)Nc3nccc(N5CCOCC5)c3CO4)CC2)C1] |
| 79 | 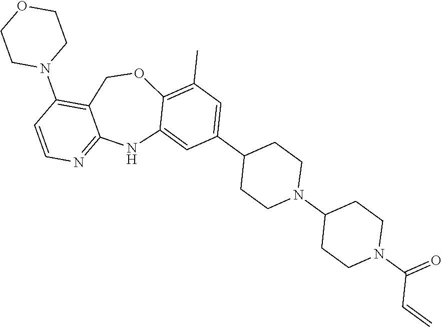 [SMILES: C=CC(=O)N1CCC(N2CCC(c3cc(C)c4c(c3)Nc3nccc(N5CCOCC5)c3CO4)CC2)CC1] |
| 80 | 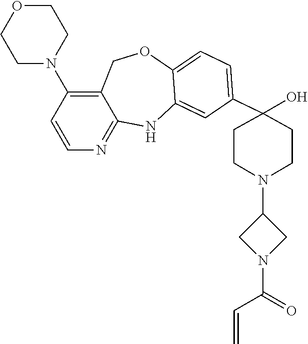 [SMILES: C=CC(=O)N1CC(N2CCC(O)(c3ccc4c(c3)Nc3nccc(N5CCOCC5)c3CO4)CC2)C1] |
| 81 | 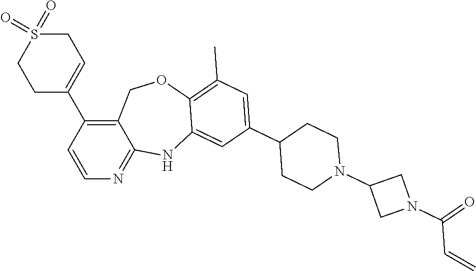 [SMILES: C=CC(=O)N1CC(N2CCC(c3cc(C)c4c(c3)Nc3nccc(C5=CCS(=O)(=O)CC5)c3CO4)CC2)C1] |
| 82 | 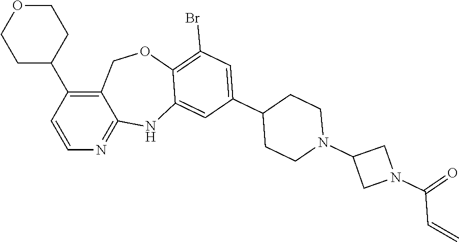 [SMILES: C=CC(=O)N1CC(N2CCC(c3cc(Br)c4c(c3)Nc3nccc(C5CCOCC5)c3CO4)CC2)C1] |
| 83 | 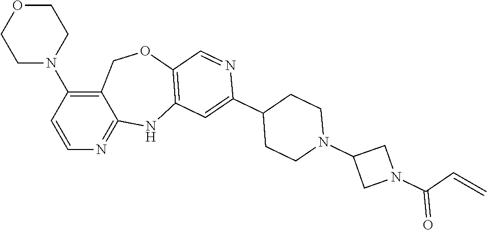 [SMILES: C=CC(=O)N1CC(N2CCC(c3cc4c(cn3)OCc3c(N5CCOCC5)ccnc3N4)CC2)C1] |
| 84 | 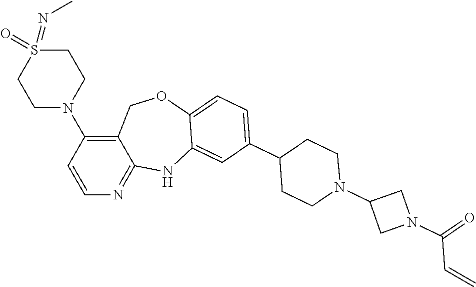 [SMILES: C=CC(=O)N1CC(N2CCC(c3ccc4c(c3)Nc3nccc(N5CCS(=O)(=NC)CC5)c3CO4)CC2)C1] |
| 85 | 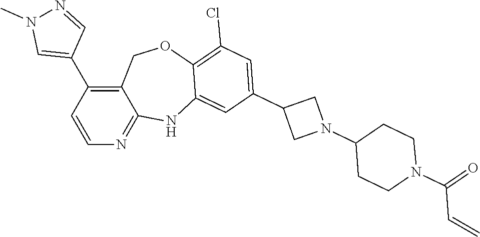 [SMILES: C=CC(=O)N1CCC(N2CC(c3cc(Cl)c4c(c3)Nc3nccc(-c5cnn(C)c5)c3CO4)C2)CC1] |
| 86 | 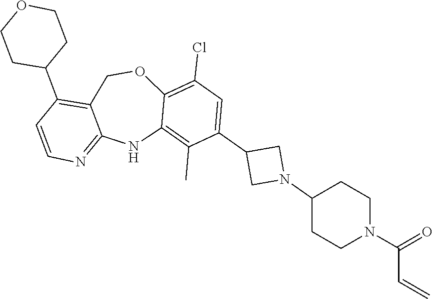 [SMILES: C=CC(=O)N1CCC(N2CC(c3cc(Cl)c4c(c3C)Nc3nccc(C5CCOCC5)c3CO4)C2)CC1] |
| 87 | 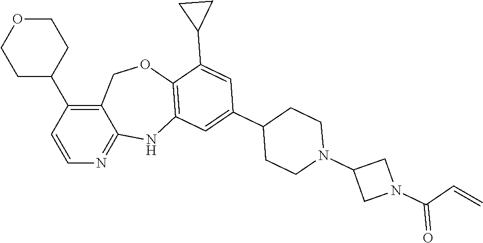 [SMILES: C=CC(=O)N1CC(N2CCC(c3cc4c(c(C5CC5)c3)OCc3c(C5CCOCC5)ccnc3N4)CC2)C1] |
| 88 | 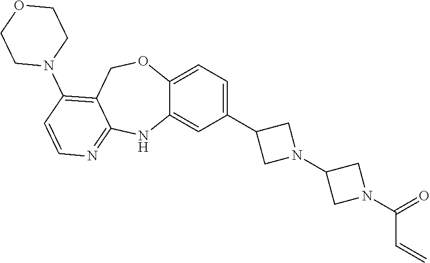 [SMILES: C=CC(=O)N1CC(N2CC(c3ccc4c(c3)Nc3nccc(N5CCOCC5)c3CO4)C2)C1] |
| 89 | 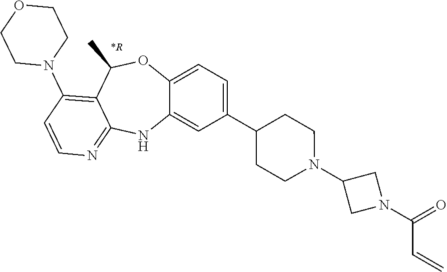 [SMILES: C=CC(=O)N1CC(N2CCC(c3ccc4c(c3)Nc3nccc(N5CCOCC5)c3[C@@H](C)O4)CC2)C1] |
| 90 | 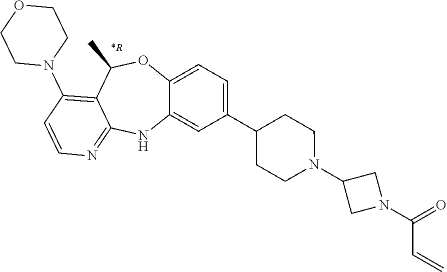 [SMILES: C=CC(=O)N1CC(N2CCC(c3ccc4c(c3)Nc3nccc(N5CCOCC5)c3[C@@H](C)O4)CC2)C1] |
| 91 | 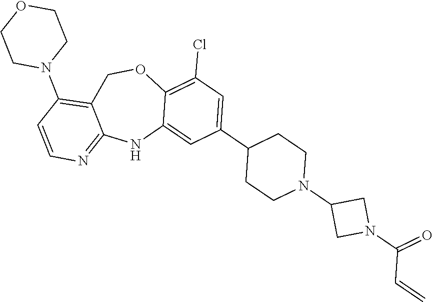 [SMILES: C=CC(=O)N1CC(N2CCC(c3cc(Cl)c4c(c3)Nc3nccc(N5CCOCC5)c3CO4)CC2)C1] |
| 92 | 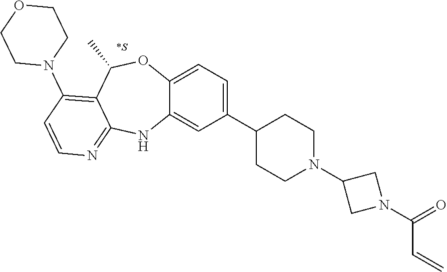 [SMILES: C=CC(=O)N1CC(N2CCC(c3ccc4c(c3)Nc3nccc(N5CCOCC5)c3[C@H](C)O4)CC2)C1] |
| 93 | 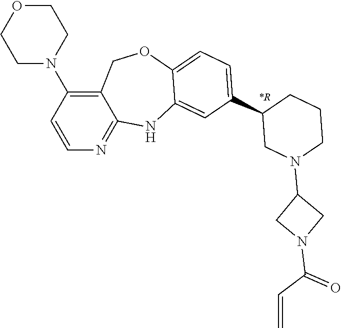 [SMILES: C=CC(=O)N1CC(N2CCC[C@H](c3ccc4c(c3)Nc3nccc(N5CCOCC5)c3CO4)C2)C1] |
| 94 | 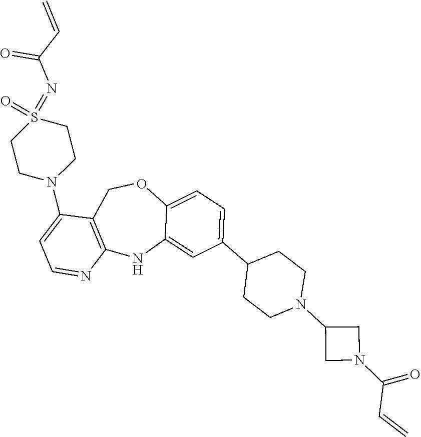 [SMILES: C=CC(=O)N=S1(=O)CCN(c2ccnc3c2COc2ccc(C4CCN(C5CN(C(=O)C=C)C5)CC4)cc2N3)CC1] |
| 95 | 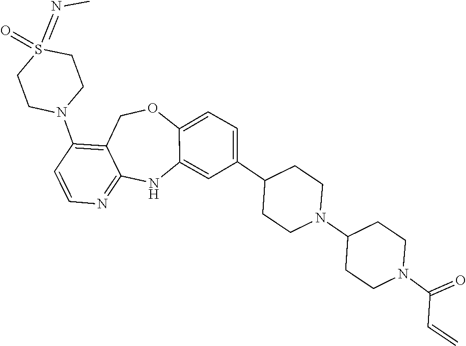 [SMILES: C=CC(=O)N1CCC(N2CCC(c3ccc4c(c3)Nc3nccc(N5CCS(=O)(=NC)CC5)c3CO4)CC2)CC1] |
| 96 | 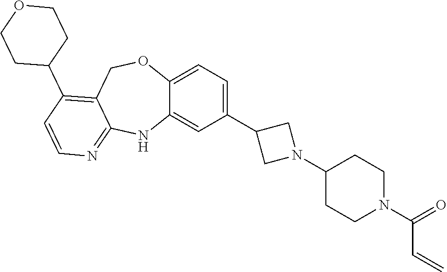 [SMILES: C=CC(=O)N1CCC(N2CC(c3ccc4c(c3)Nc3nccc(C5CCOCC5)c3CO4)C2)CC1] |
| 97 | 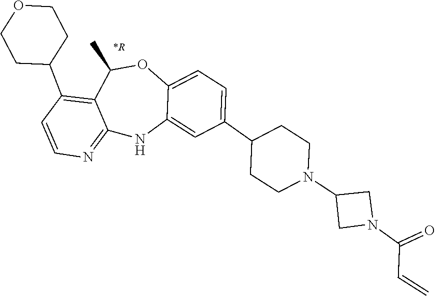 [SMILES: C=CC(=O)N1CC(N2CCC(c3ccc4c(c3)Nc3nccc(C5CCOCC5)c3[C@@H](C)O4)CC2)C1] |
| 98 | 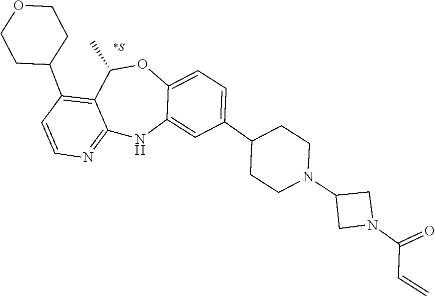 [SMILES: C=CC(=O)N1CC(N2CCC(c3ccc4c(c3)Nc3nccc(C5CCOCC5)c3[C@H](C)O4)CC2)C1] |
| 99 | 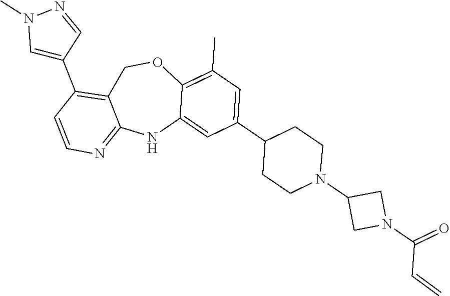 [SMILES: C=CC(=O)N1CC(N2CCC(c3cc(C)c4c(c3)Nc3nccc(-c5cnn(C)c5)c3CO4)CC2)C1] |
| 100 | 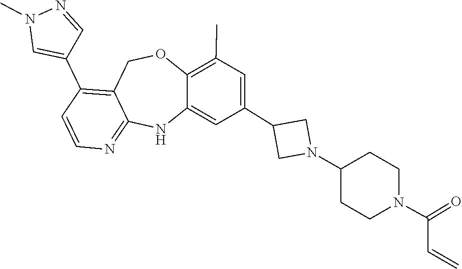 [SMILES: C=CC(=O)N1CCC(N2CC(c3cc(C)c4c(c3)Nc3nccc(-c5cnn(C)c5)c3CO4)C2)CC1] |
| 101 | 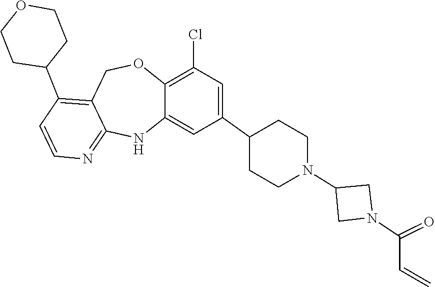 [SMILES: C=CC(=O)N1CC(N2CCC(c3cc(Cl)c4c(c3)Nc3nccc(C5CCOCC5)c3CO4)CC2)C1] |
| 102 | 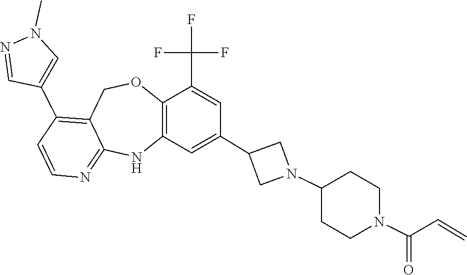 [SMILES: C=CC(=O)N1CCC(N2CC(c3cc4c(c(C(F)(F)F)c3)OCc3c(-c5cnn(C)c5)ccnc3N4)C2)CC1] |
| 103 | 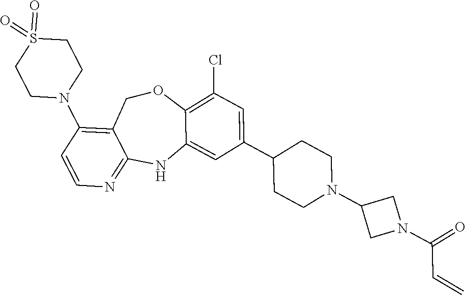 [SMILES: C=CC(=O)N1CC(N2CCC(c3cc(Cl)c4c(c3)Nc3nccc(N5CCS(=O)(=O)CC5)c3CO4)CC2)C1] |
| 104 | 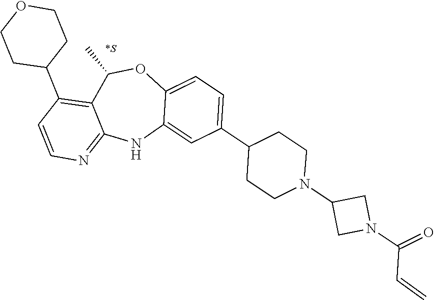 [SMILES: C=CC(=O)N1CC(N2CCC(c3ccc4c(c3)Nc3nccc(C5CCOCC5)c3[C@H](C)O4)CC2)C1] |
| 105 | 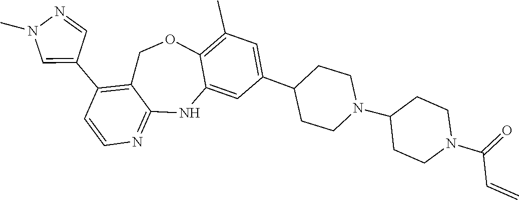 [SMILES: C=CC(=O)N1CCC(N2CCC(c3cc(C)c4c(c3)Nc3nccc(-c5cnn(C)c5)c3CO4)CC2)CC1] |
| 106 | 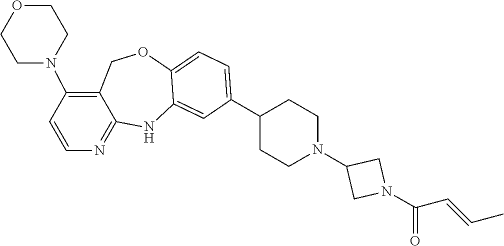 [SMILES: C/C=C/C(=O)N1CC(N2CCC(c3ccc4c(c3)Nc3nccc(N5CCOCC5)c3CO4)CC2)C1] |
| 107 | 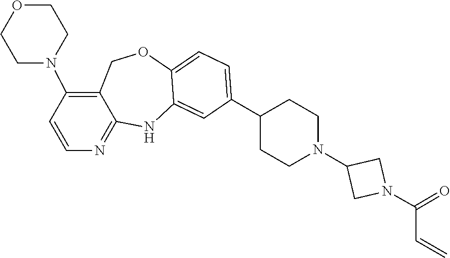 [SMILES: C=CC(=O)N1CC(N2CCC(c3ccc4c(c3)Nc3nccc(N5CCOCC5)c3CO4)CC2)C1] |
| 108 | 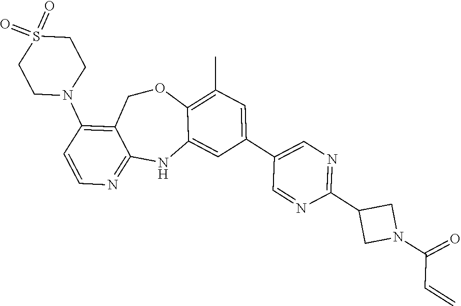 [SMILES: C=CC(=O)N1CC(c2ncc(-c3cc(C)c4c(c3)Nc3nccc(N5CCS(=O)(=O)CC5)c3CO4)cn2)C1] |
| 109 | 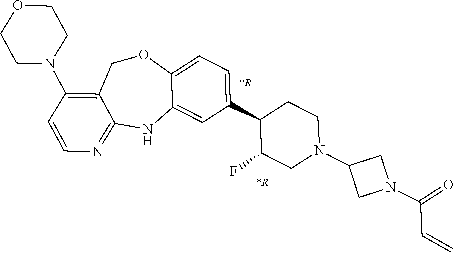 [SMILES: C=CC(=O)N1CC(N2CC[C@H](c3ccc4c(c3)Nc3nccc(N5CCOCC5)c3CO4)[C@@H](F)C2)C1] |
| 110 | 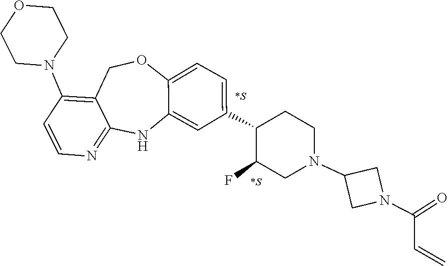 [SMILES: C=CC(=O)N1CC(N2CC[C@@H](c3ccc4c(c3)Nc3nccc(N5CCOCC5)c3CO4)[C@H](F)C2)C1] |
| 111 | 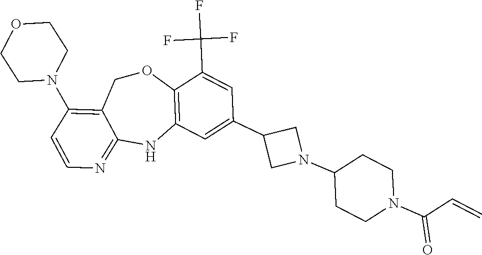 [SMILES: C=CC(=O)N1CCC(N2CC(c3cc4c(c(C(F)(F)F)c3)OCc3c(N5CCOCC5)ccnc3N4)C2)CC1] |
| 112 | 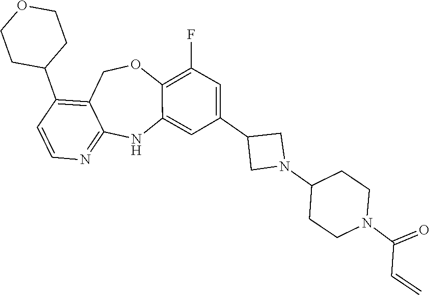 [SMILES: C=CC(=O)N1CCC(N2CC(c3cc(F)c4c(c3)Nc3nccc(C5CCOCC5)c3CO4)C2)CC1] |
| 113 | 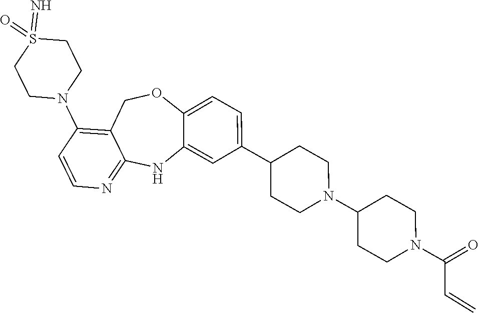 |
| 114 | 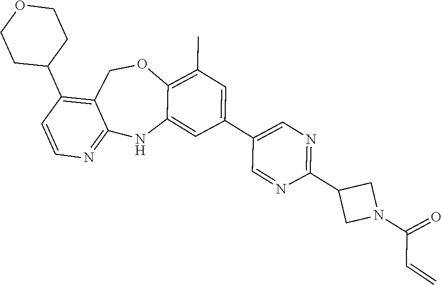 [SMILES: C=CC(=O)N1CC(c2ncc(-c3cc(C)c4c(c3)Nc3nccc(C5CCOCC5)c3CO4)cn2)C1] |
| 115 | 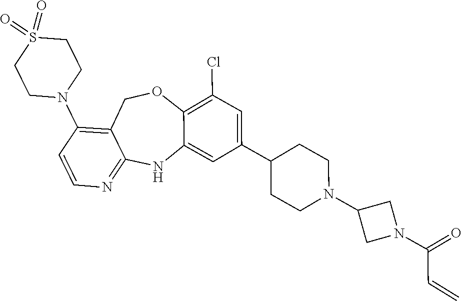 [SMILES: C=CC(=O)N1CC(N2CCC(c3cc(Cl)c4c(c3)Nc3nccc(N5CCS(=O)(=O)CC5)c3CO4)CC2)C1] |
| 116 | 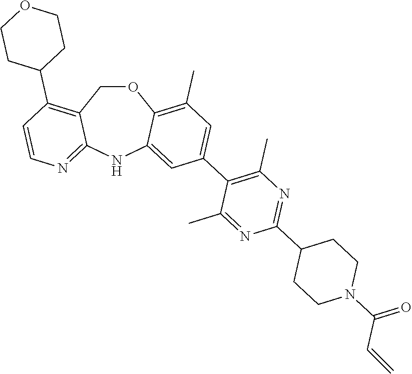 [SMILES: C=CC(=O)N1CCC(c2nc(C)c(-c3cc(C)c4c(c3)Nc3nccc(C5CCOCC5)c3CO4)c(C)n2)CC1] |
| 117 | 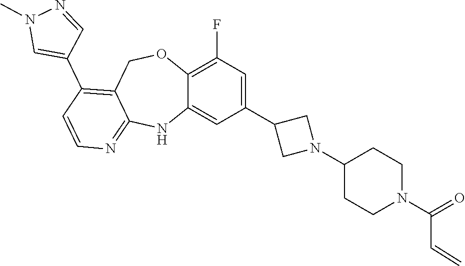 [SMILES: C=CC(=O)N1CCC(N2CC(c3cc(F)c4c(c3)Nc3nccc(-c5cnn(C)c5)c3CO4)C2)CC1] |
| 118 | 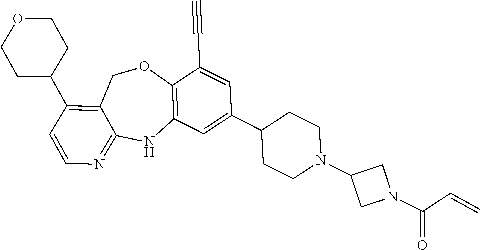 [SMILES: C#Cc1cc(C2CCN(C3CN(C(=O)C=C)C3)CC2)cc2c1OCc1c(C3CCOCC3)ccnc1N2] |
| 119 | 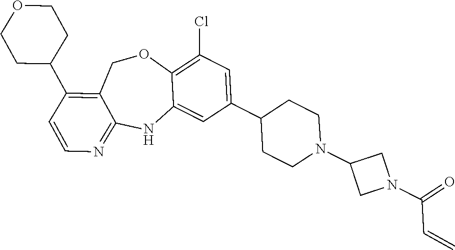 [SMILES: C=CC(=O)N1CC(N2CCC(c3cc(Cl)c4c(c3)Nc3nccc(C5CCOCC5)c3CO4)CC2)C1] |
| 120 | 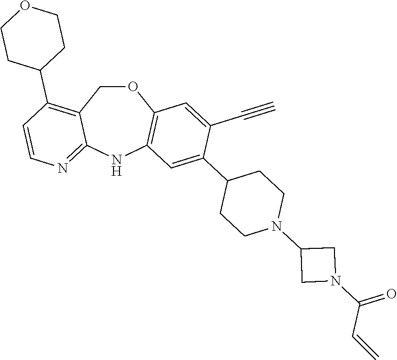 [SMILES: C#Cc1cc2c(cc1C1CCN(C3CN(C(=O)C=C)C3)CC1)Nc1nccc(C3CCOCC3)c1CO2] |
| 121 | 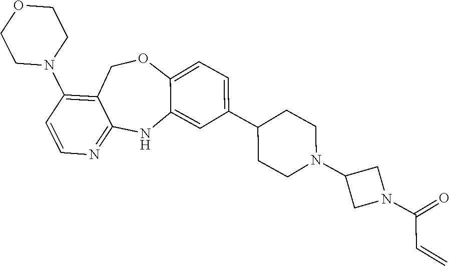 [SMILES: C=CC(=O)N1CC(N2CCC(c3ccc4c(c3)Nc3nccc(N5CCOCC5)c3CO4)CC2)C1] |
| 122 | 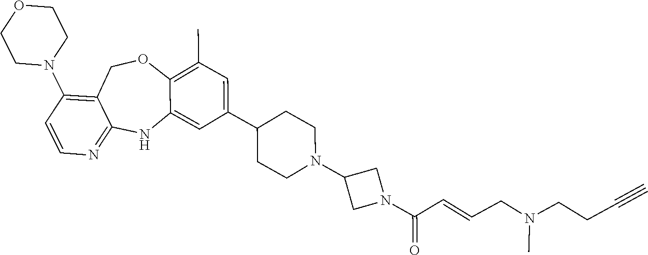 [SMILES: C#CCCN(C)C/C=C/C(=O)N1CC(N2CCC(c3cc(C)c4c(c3)Nc3nccc(N5CCOCC5)c3CO4)CC2)C1] |
| 123 | 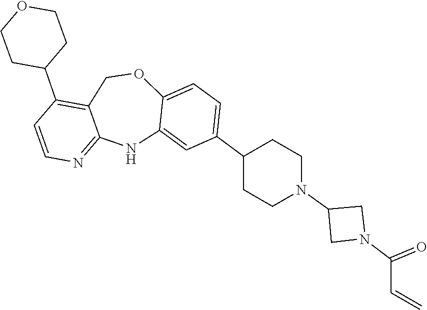 [SMILES: C=CC(=O)N1CC(N2CCC(c3ccc4c(c3)Nc3nccc(C5CCOCC5)c3CO4)CC2)C1] |
| 124 | 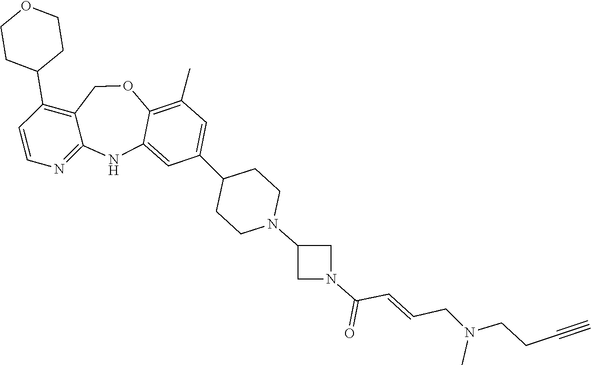 [SMILES: C#CCCN(C)C/C=C/C(=O)N1CC(N2CCC(c3cc(C)c4c(c3)Nc3nccc(C5CCOCC5)c3CO4)CC2)C1] |
| 125 | 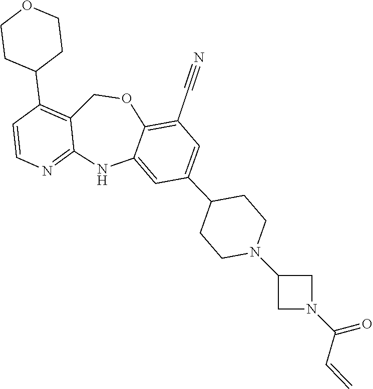 [SMILES: C=CC(=O)N1CC(N2CCC(c3cc(C#N)c4c(c3)Nc3nccc(C5CCOCC5)c3CO4)CC2)C1] |
| 126 | 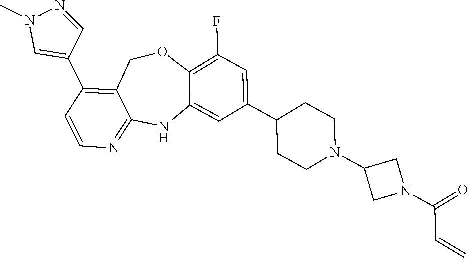 [SMILES: C=CC(=O)N1CC(N2CCC(c3cc(F)c4c(c3)Nc3nccc(-c5cnn(C)c5)c3CO4)CC2)C1] |
| 127 | 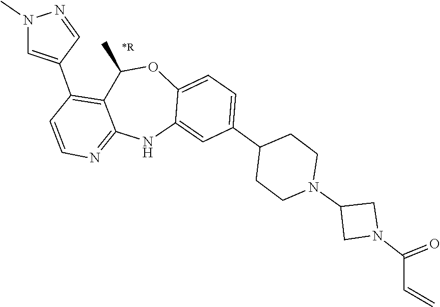 [SMILES: C=CC(=O)N1CC(N2CCC(c3ccc4c(c3)Nc3nccc(-c5cnn(C)c5)c3[C@@H](C)O4)CC2)C1] |
| 128 | 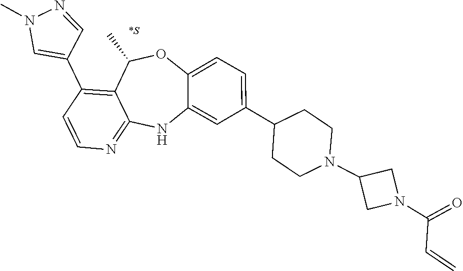 [SMILES: C=CC(=O)N1CC(N2CCC(c3ccc4c(c3)Nc3nccc(-c5cnn(C)c5)c3[C@H](C)O4)CC2)C1] |
| 129 | 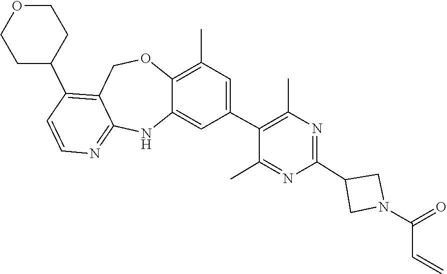 [SMILES: C=CC(=O)N1CC(c2nc(C)c(-c3cc(C)c4c(c3)Nc3nccc(C5CCOCC5)c3CO4)c(C)n2)C1] |
| 130 | 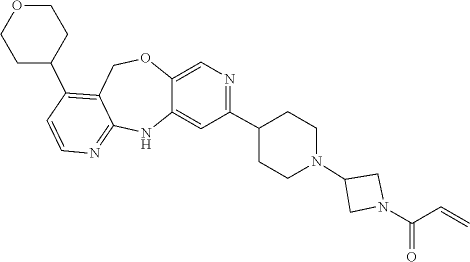 [SMILES: C=CC(=O)N1CC(N2CCC(c3cc4c(cn3)OCc3c(C5CCOCC5)ccnc3N4)CC2)C1] |
| 131 | 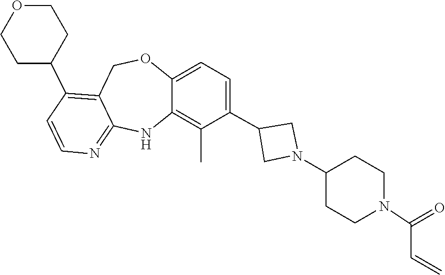 [SMILES: C=CC(=O)N1CCC(N2CC(c3ccc4c(c3C)Nc3nccc(C5CCOCC5)c3CO4)C2)CC1] |
| 132 | 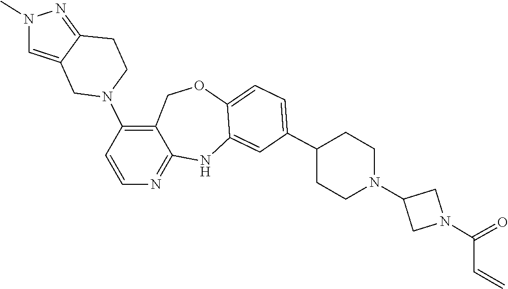 [SMILES: C=CC(=O)N1CC(N2CCC(c3ccc4c(c3)Nc3nccc(N5CCc6nn(C)cc6C5)c3CO4)CC2)C1] |
| 133 | 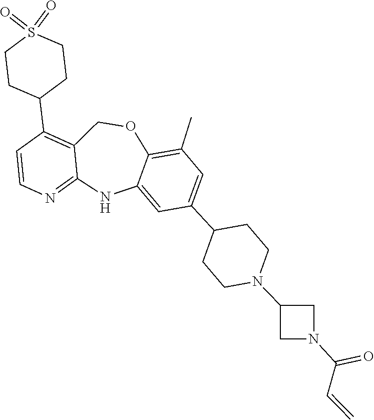 [SMILES: C=CC(=O)N1CC(N2CCC(c3cc(C)c4c(c3)Nc3nccc(C5CCS(=O)(=O)CC5)c3CO4)CC2)C1] |
| 134 | 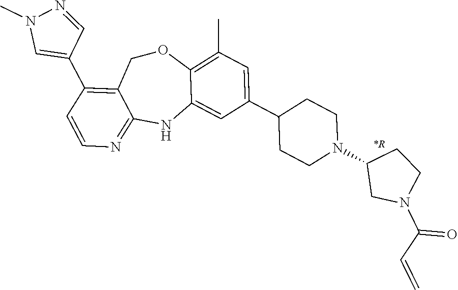 [SMILES: C=CC(=O)N1CC[C@@H](N2CCC(c3cc(C)c4c(c3)Nc3nccc(-c5cnn(C)c5)c3CO4)CC2)C1] |
| 135 | 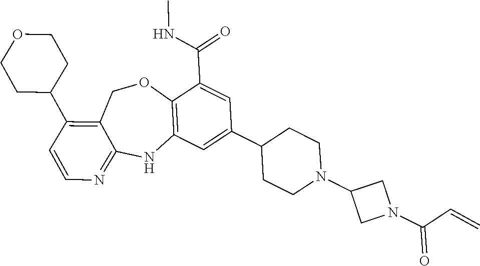 [SMILES: C=CC(=O)N1CC(N2CCC(c3cc4c(c(C(=O)NC)c3)OCc3c(C5CCOCC5)ccnc3N4)CC2)C1] |
| 136 | 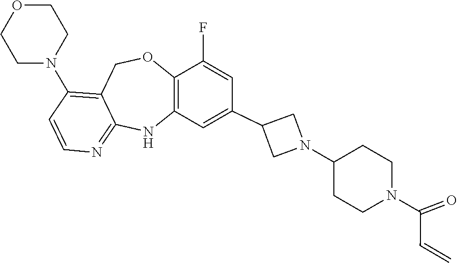 [SMILES: C=CC(=O)N1CCC(N2CC(c3cc(F)c4c(c3)Nc3nccc(N5CCOCC5)c3CO4)C2)CC1] |
| 137 | 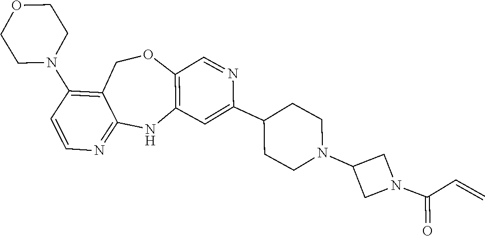 [SMILES: C=CC(=O)N1CC(N2CCC(c3cc4c(cn3)OCc3c(N5CCOCC5)ccnc3N4)CC2)C1] |
| 138 | 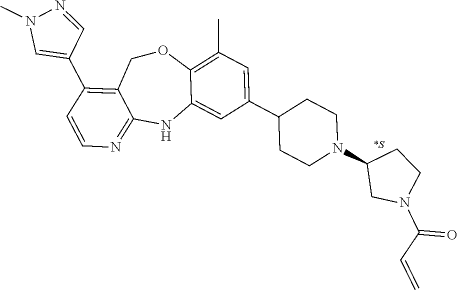 [SMILES: C=CC(=O)N1CC[C@H](N2CCC(c3cc(C)c4c(c3)Nc3nccc(-c5cnn(C)c5)c3CO4)CC2)C1] |
| 139 | 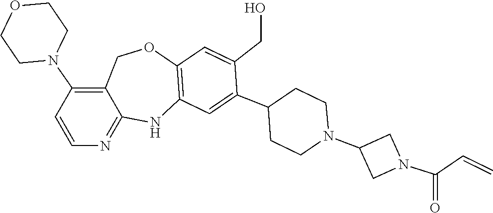 [SMILES: C=CC(=O)N1CC(N2CCC(c3cc4c(cc3CO)OCc3c(N5CCOCC5)ccnc3N4)CC2)C1] |
| 140 | 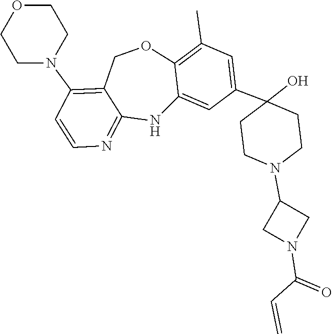 [SMILES: C=CC(=O)N1CC(N2CCC(O)(c3cc(C)c4c(c3)Nc3nccc(N5CCOCC5)c3CO4)CC2)C1] |
| 141 | 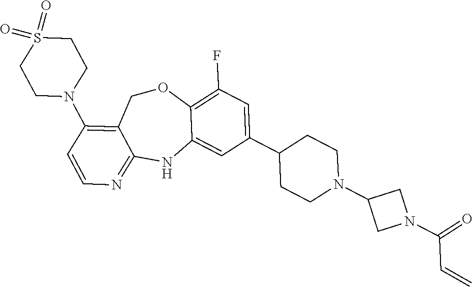 [SMILES: C=CC(=O)N1CC(N2CCC(c3cc(F)c4c(c3)Nc3nccc(N5CCS(=O)(=O)CC5)c3CO4)CC2)C1] |
| 142 | 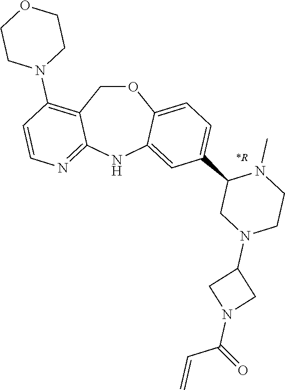 [SMILES: C=CC(=O)N1CC(N2CCN(C)[C@H](c3ccc4c(c3)Nc3nccc(N5CCOCC5)c3CO4)C2)C1] |
| 143 | 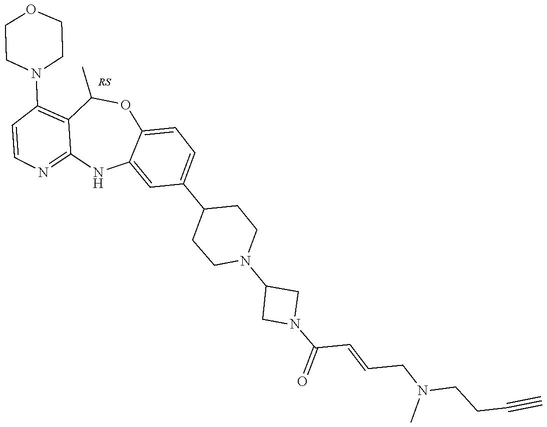 |
| 144 | 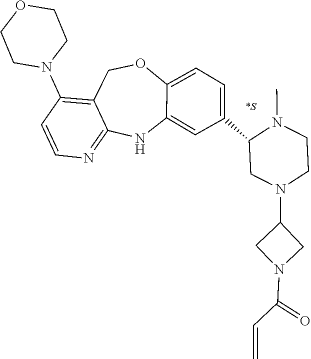 [SMILES: C=CC(=O)N1CC(N2CCN(C)[C@@H](c3ccc4c(c3)Nc3nccc(N5CCOCC5)c3CO4)C2)C1] |
| 145 | 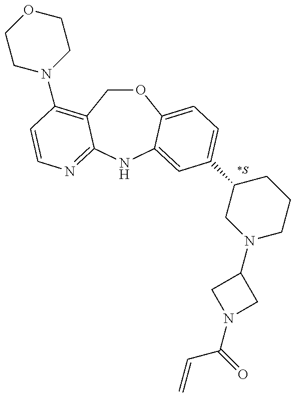 [SMILES: C=CC(=O)N1CC(N2CCC[C@@H](c3ccc4c(c3)Nc3nccc(N5CCOCC5)c3CO4)C2)C1] |
| 146 | 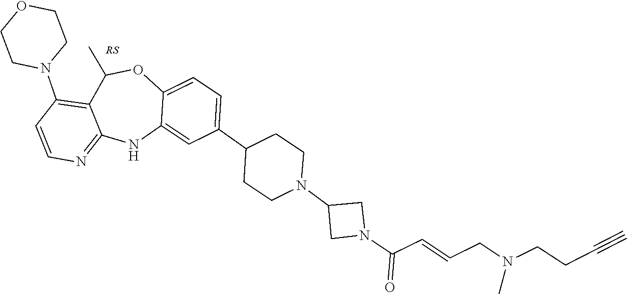 [SMILES: C#CCCN(C)C/C=C/C(=O)N1CC(N2CCC(c3ccc4c(c3)Nc3nccc(N5CCOCC5)c3C(C)O4)CC2)C1] |
| 147 | 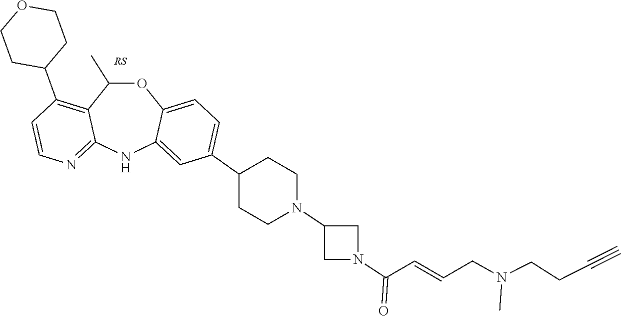 [SMILES: C#CCCN(C)C/C=C/C(=O)N1CC(N2CCC(c3ccc4c(c3)Nc3nccc(C5CCOCC5)c3C(C)O4)CC2)C1] |
| 148 | 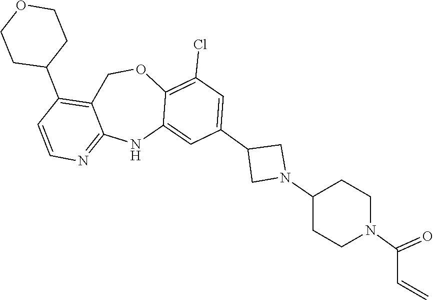 [SMILES: C=CC(=O)N1CCC(N2CC(c3cc(Cl)c4c(c3)Nc3nccc(C5CCOCC5)c3CO4)C2)CC1] |
| 149 | 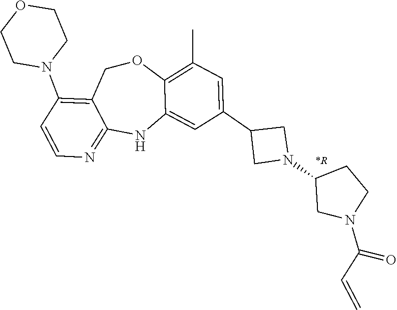 [SMILES: C=CC(=O)N1CC[C@@H](N2CC(c3cc(C)c4c(c3)Nc3nccc(N5CCOCC5)c3CO4)C2)C1] |
| 150 | 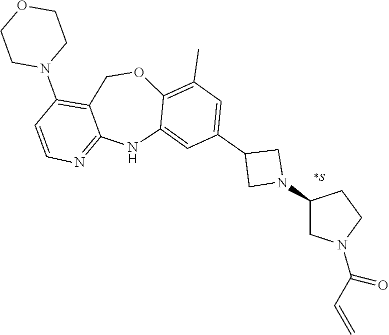 [SMILES: C=CC(=O)N1CC[C@H](N2CC(c3cc(C)c4c(c3)Nc3nccc(N5CCOCC5)c3CO4)C2)C1] |
| 151 | 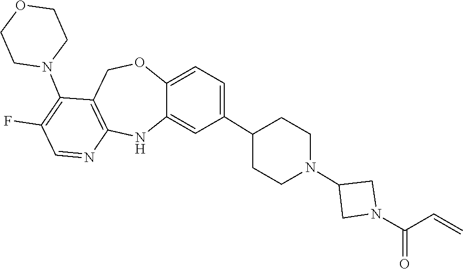 [SMILES: C=CC(=O)N1CC(N2CCC(c3ccc4c(c3)Nc3ncc(F)c(N5CCOCC5)c3CO4)CC2)C1] |
| 152 | 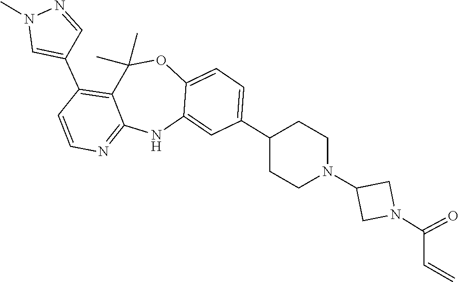 [SMILES: C=CC(=O)N1CC(N2CCC(c3ccc4c(c3)Nc3nccc(-c5cnn(C)c5)c3C(C)(C)O4)CC2)C1] |
| 153 | 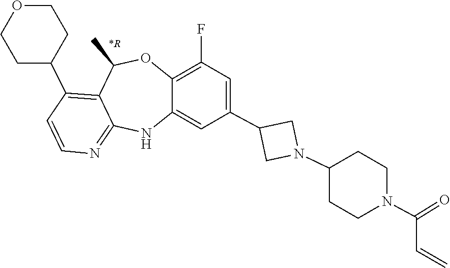 [SMILES: C=CC(=O)N1CCC(N2CC(c3cc(F)c4c(c3)Nc3nccc(C5CCOCC5)c3[C@@H](C)O4)C2)CC1] |
| 154 | 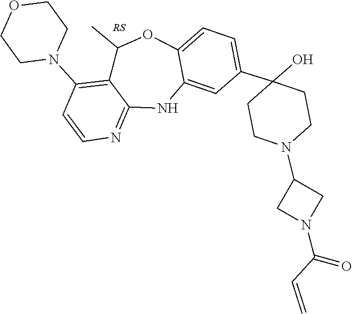 [SMILES: C=CC(=O)N1CC(N2CCC(O)(c3ccc4c(c3)Nc3nccc(N5CCOCC5)c3C(C)O4)CC2)C1] |
| 155 | 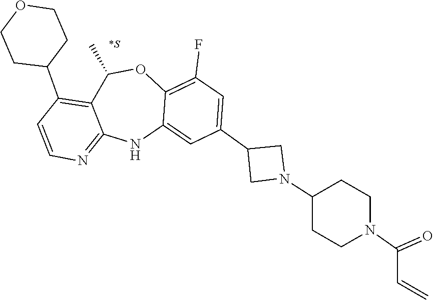 [SMILES: C=CC(=O)N1CCC(N2CC(c3cc(F)c4c(c3)Nc3nccc(C5CCOCC5)c3[C@H](C)O4)C2)CC1] |
| 156 | 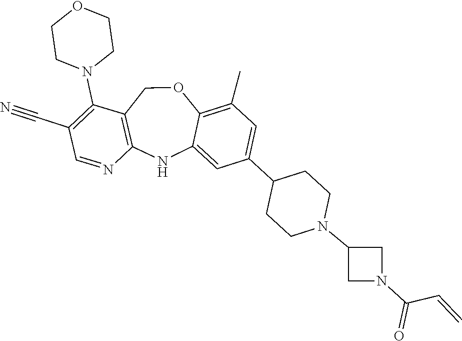 [SMILES: C=CC(=O)N1CC(N2CCC(c3cc(C)c4c(c3)Nc3ncc(C#N)c(N5CCOCC5)c3CO4)CC2)C1] |
| 157 | 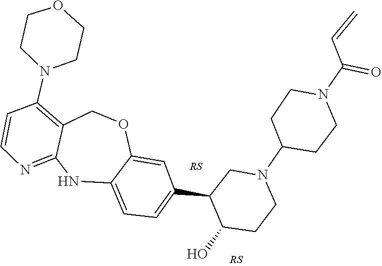 [SMILES: C=CC(=O)N1CCC(N2CC[C@H](O)[C@@H](c3ccc4c(c3)OCc3c(N5CCOCC5)ccnc3N4)C2)CC1] |
| 158 | 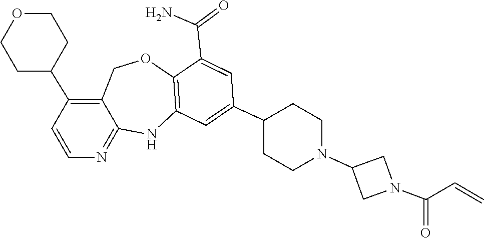 [SMILES: C=CC(=O)N1CC(N2CCC(c3cc4c(c(C(N)=O)c3)OCc3c(C5CCOCC5)ccnc3N4)CC2)C1] |
| 159 | 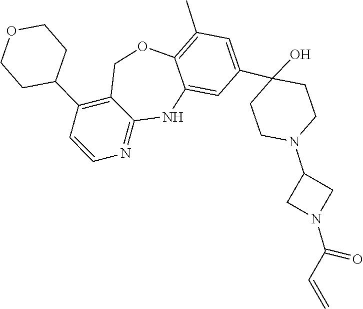 [SMILES: C=CC(=O)N1CC(N2CCC(O)(c3cc(C)c4c(c3)Nc3nccc(C5CCOCC5)c3CO4)CC2)C1] |
| 160 | 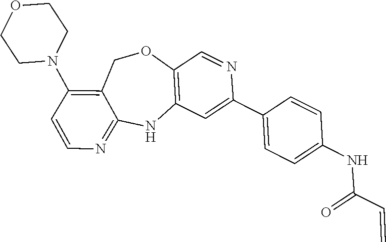 [SMILES: C=CC(=O)Nc1ccc(-c2cc3c(cn2)OCc2c(N4CCOCC4)ccnc2N3)cc1] |
| 161 | 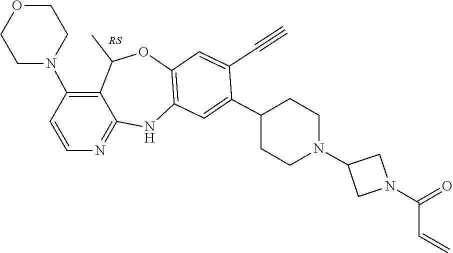 |
| 162 | 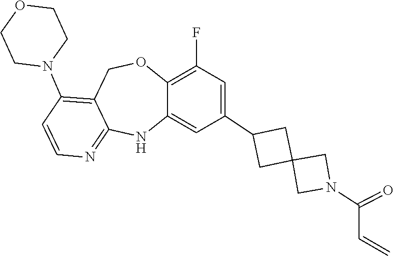 |
| 163 | 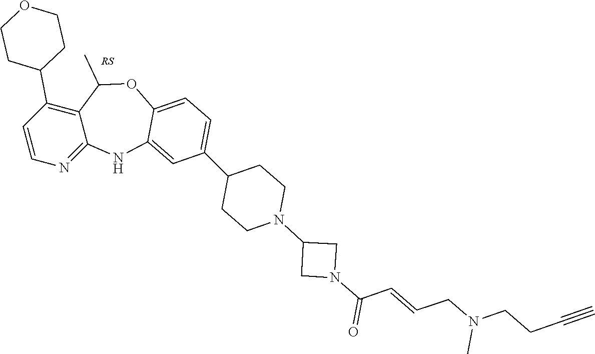 [SMILES: C#CCCN(C)C/C=C/C(=O)N1CC(N2CCC(c3ccc4c(c3)Nc3nccc(C5CCOCC5)c3C(C)O4)CC2)C1] |
| 164 | 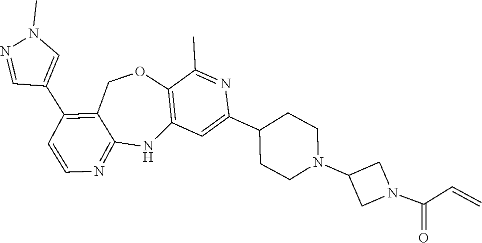 [SMILES: C=CC(=O)N1CC(N2CCC(c3cc4c(c(C)n3)OCc3c(-c5cnn(C)c5)ccnc3N4)CC2)C1] |
| 165 | 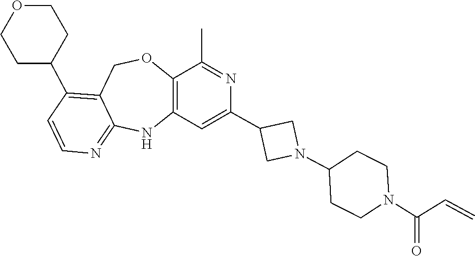 [SMILES: C=CC(=O)N1CCC(N2CC(c3cc4c(c(C)n3)OCc3c(C5CCOCC5)ccnc3N4)C2)CC1] |
| 166 | 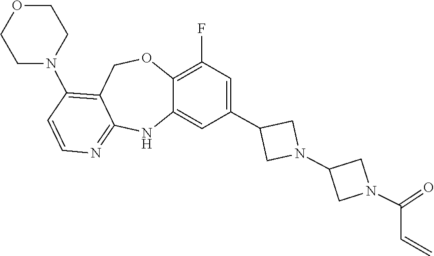 [SMILES: C=CC(=O)N1CC(N2CC(c3cc(F)c4c(c3)Nc3nccc(N5CCOCC5)c3CO4)C2)C1] |
| 167 | 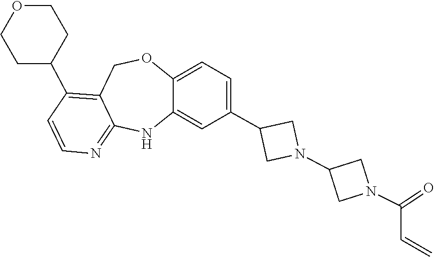 [SMILES: C=CC(=O)N1CC(N2CC(c3ccc4c(c3)Nc3nccc(C5CCOCC5)c3CO4)C2)C1] |
| 168 | 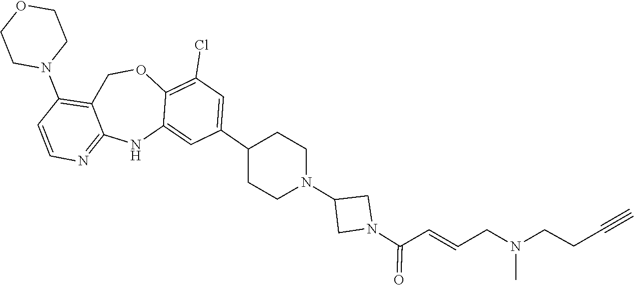 [SMILES: C#CCCN(C)C/C=C/C(=O)N1CC(N2CCC(c3cc(Cl)c4c(c3)Nc3nccc(N5CCOCC5)c3CO4)CC2)C1] |
| 169 | 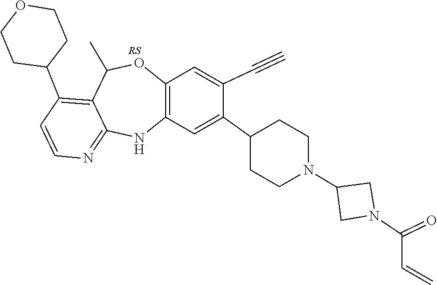 |
| 170 | 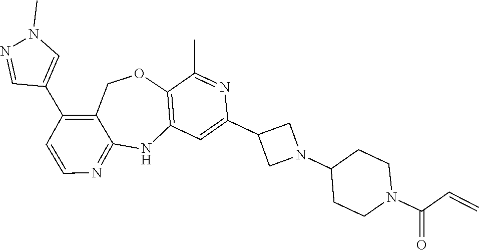 [SMILES: C=CC(=O)N1CCC(N2CC(c3cc4c(c(C)n3)OCc3c(-c5cnn(C)c5)ccnc3N4)C2)CC1] |
| 171 | 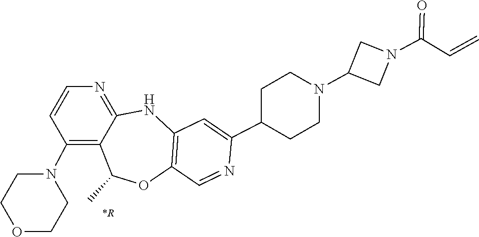 [SMILES: C=CC(=O)N1CC(N2CCC(c3cc4c(cn3)O[C@H](C)c3c(N5CCOCC5)ccnc3N4)CC2)C1] |
| 172 | 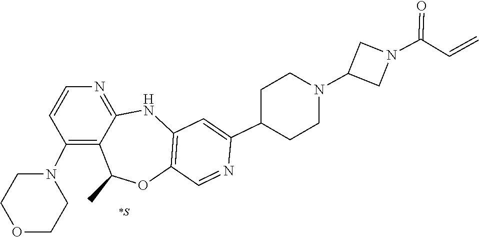 [SMILES: C=CC(=O)N1CC(N2CCC(c3cc4c(cn3)O[C@@H](C)c3c(N5CCOCC5)ccnc3N4)CC2)C1] |
| 173 | 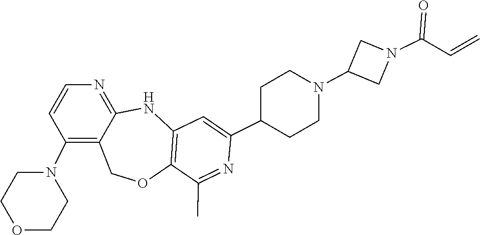 [SMILES: C=CC(=O)N1CC(N2CCC(c3cc4c(c(C)n3)OCc3c(N5CCOCC5)ccnc3N4)CC2)C1] |
| 174 | 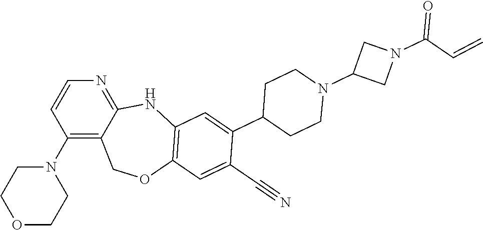 [SMILES: C=CC(=O)N1CC(N2CCC(c3cc4c(cc3C#N)OCc3c(N5CCOCC5)ccnc3N4)CC2)C1] |
| 175 | 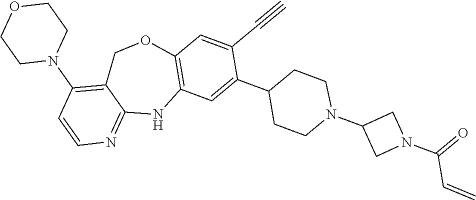 [SMILES: C#Cc1cc2c(cc1C1CCN(C3CN(C(=O)C=C)C3)CC1)Nc1nccc(N3CCOCC3)c1CO2] |
| 176 | 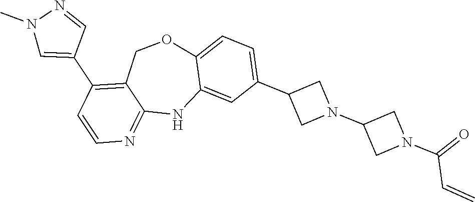 |
| 177 | 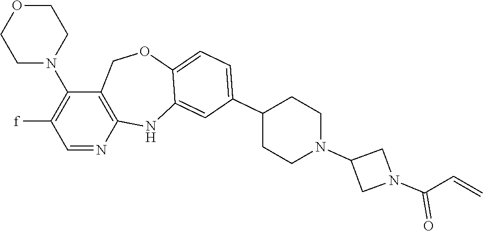 |
| 178 | 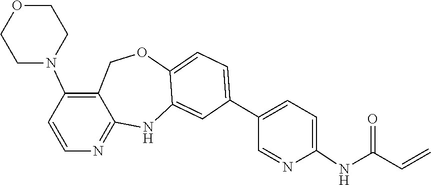 [SMILES: C=CC(=O)Nc1ccc(-c2ccc3c(c2)Nc2nccc(N4CCOCC4)c2CO3)cn1] |
| 179 | 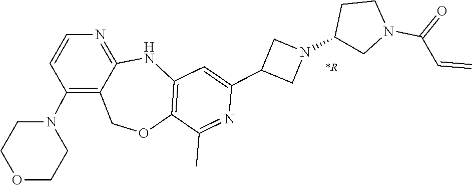 [SMILES: C=CC(=O)N1CC[C@@H](N2CC(c3cc4c(c(C)n3)OCc3c(N5CCOCC5)ccnc3N4)C2)C1] |
| 180 | 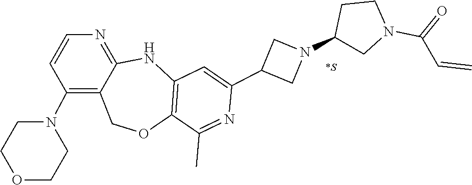 [SMILES: C=CC(=O)N1CC[C@H](N2CC(c3cc4c(c(C)n3)OCc3c(N5CCOCC5)ccnc3N4)C2)C1] |
| 181 | 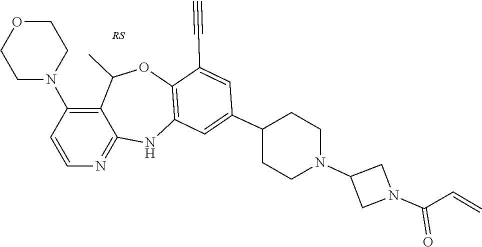 [SMILES: C#Cc1cc(C2CCN(C3CN(C(=O)C=C)C3)CC2)cc2c1OC(C)c1c(N3CCOCC3)ccnc1N2] |
| 182 | 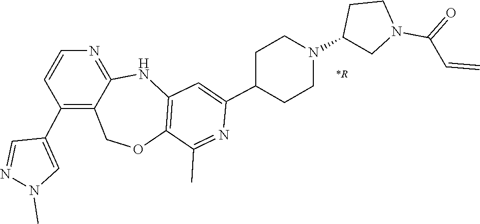 [SMILES: C=CC(=O)N1CC[C@@H](N2CCC(c3cc4c(c(C)n3)OCc3c(-c5cnn(C)c5)ccnc3N4)CC2)C1] |
| 183 | 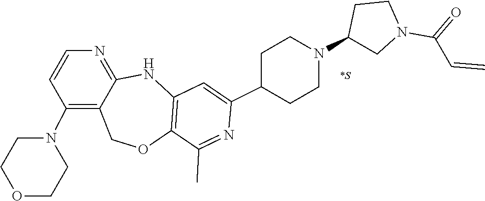 [SMILES: C=CC(=O)N1CC[C@H](N2CCC(c3cc4c(c(C)n3)OCc3c(N5CCOCC5)ccnc3N4)CC2)C1] |
| 184 | 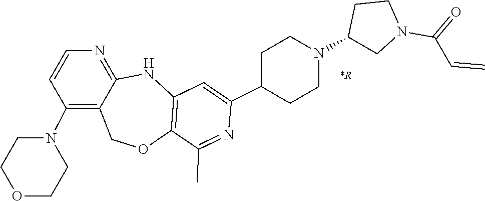 [SMILES: C=CC(=O)N1CC[C@@H](N2CCC(c3cc4c(c(C)n3)OCc3c(N5CCOCC5)ccnc3N4)CC2)C1] |
| 185 | 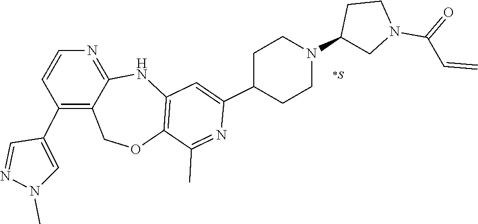 [SMILES: C=CC(=O)N1CC[C@H](N2CCC(c3cc4c(c(C)n3)OCc3c(-c5cnn(C)c5)ccnc3N4)CC2)C1] |
| 186 | 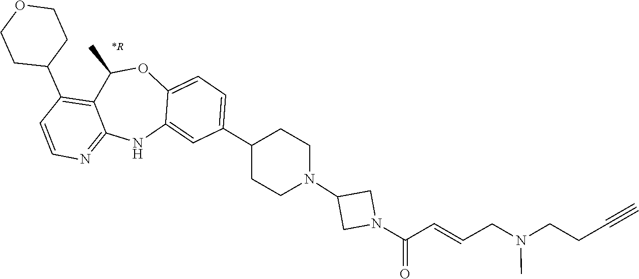 [SMILES: C#CCCN(C)C/C=C/C(=O)N1CC(N2CCC(c3ccc4c(c3)Nc3nccc(C5CCOCC5)c3[C@@H](C)O4)CC2)C1] |
| 187 | 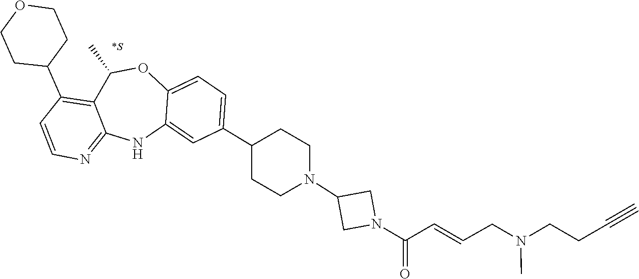 [SMILES: C#CCCN(C)C/C=C/C(=O)N1CC(N2CCC(c3ccc4c(c3)Nc3nccc(C5CCOCC5)c3[C@H](C)O4)CC2)C1] |
| 188 | 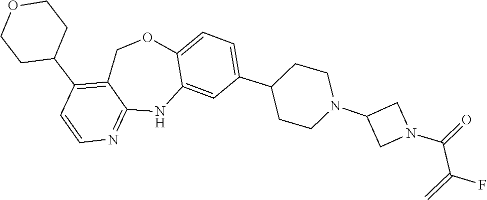 [SMILES: C=C(F)C(=O)N1CC(N2CCC(c3ccc4c(c3)Nc3nccc(C5CCOCC5)c3CO4)CC2)C1] |
| 189 | 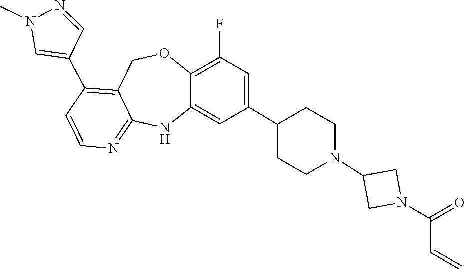 [SMILES: C=CC(=O)N1CC(N2CCC(c3cc(F)c4c(c3)Nc3nccc(-c5cnn(C)c5)c3CO4)CC2)C1] |
| 190 | 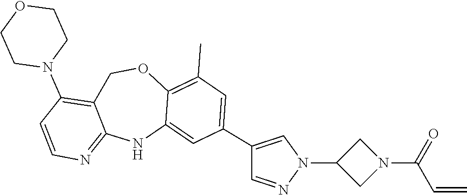 [SMILES: C=CC(=O)N1CC(n2cc(-c3cc(C)c4c(c3)Nc3nccc(N5CCOCC5)c3CO4)cn2)C1] |
| 191 | 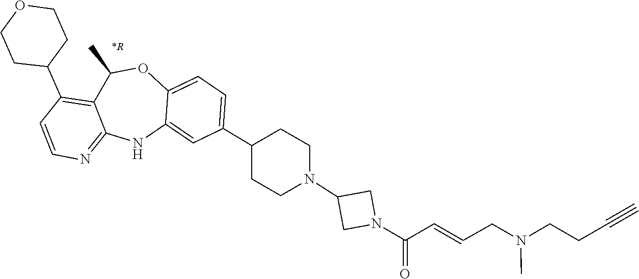 [SMILES: C#CCCN(C)C/C=C/C(=O)N1CC(N2CCC(c3ccc4c(c3)Nc3nccc(C5CCOCC5)c3[C@@H](C)O4)CC2)C1] |
| 192 | 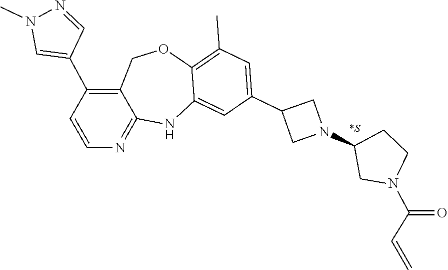 [SMILES: C=CC(=O)N1CC[C@H](N2CC(c3cc(C)c4c(c3)Nc3nccc(-c5cnn(C)c5)c3CO4)C2)C1] |
| 193 | 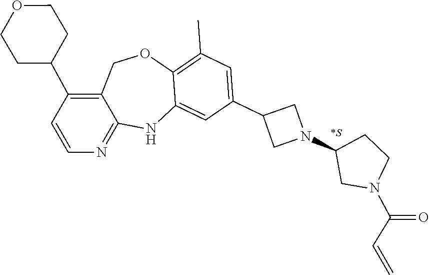 [SMILES: C=CC(=O)N1CC[C@H](N2CC(c3cc(C)c4c(c3)Nc3nccc(C5CCOCC5)c3CO4)C2)C1] |
| 194 | 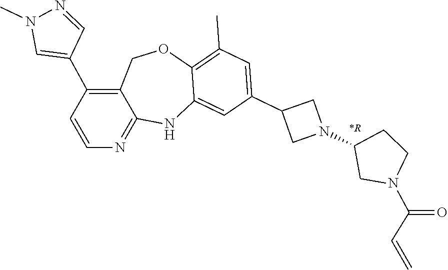 [SMILES: C=CC(=O)N1CC[C@@H](N2CC(c3cc(C)c4c(c3)Nc3nccc(-c5cnn(C)c5)c3CO4)C2)C1] |
| 195 | 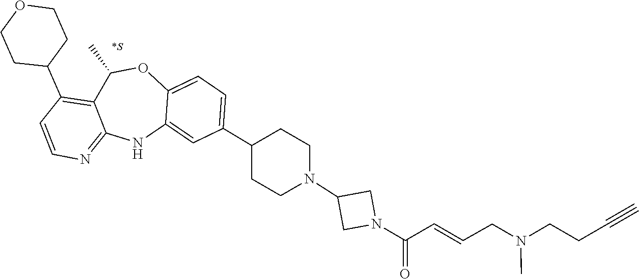 [SMILES: C#CCCN(C)C/C=C/C(=O)N1CC(N2CCC(c3ccc4c(c3)Nc3nccc(C5CCOCC5)c3[C@H](C)O4)CC2)C1] |
| 196 | 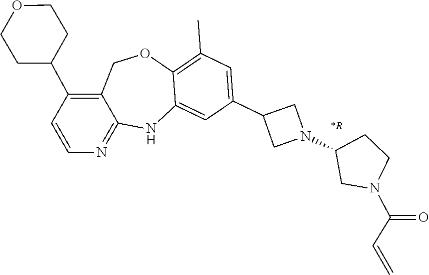 [SMILES: C=CC(=O)N1CC[C@@H](N2CC(c3cc(C)c4c(c3)Nc3nccc(C5CCOCC5)c3CO4)C2)C1] |
| 197 | 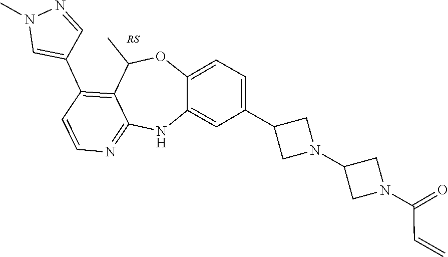 [SMILES: C=CC(=O)N1CC(N2CC(c3ccc4c(c3)Nc3nccc(-c5cnn(C)c5)c3C(C)O4)C2)C1] |
| 198 | 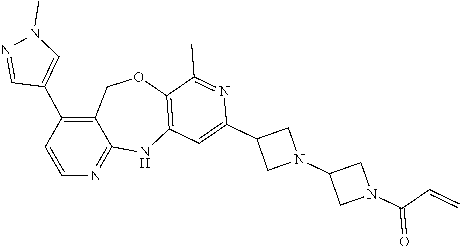 [SMILES: C=CC(=O)N1CC(N2CC(c3cc4c(c(C)n3)OCc3c(-c5cnn(C)c5)ccnc3N4)C2)C1] |
| 199 | 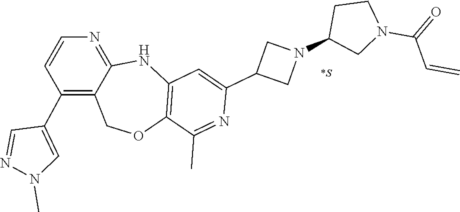 [SMILES: C=CC(=O)N1CC[C@H](N2CC(c3cc4c(c(C)n3)OCc3c(-c5cnn(C)c5)ccnc3N4)C2)C1] |
| 200 | 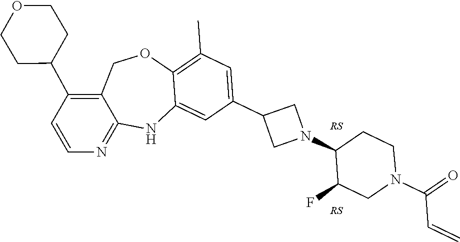 [SMILES: C=CC(=O)N1CC[C@H](N2CC(c3cc(C)c4c(c3)Nc3nccc(C5CCOCC5)c3CO4)C2)[C@H](F)C1] |
| 201 | 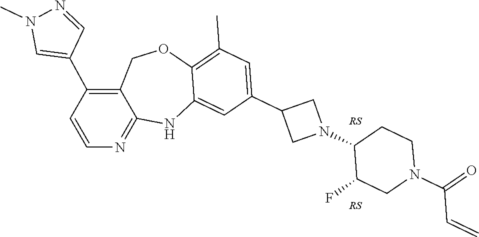 [SMILES: C=CC(=O)N1CC[C@@H](N2CC(c3cc(C)c4c(c3)Nc3nccc(-c5cnn(C)c5)c3CO4)C2)[C@@H](F)C1] |
| 202 | 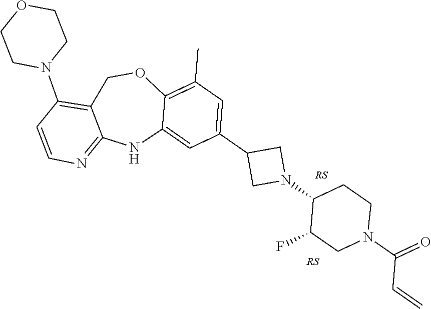 [SMILES: C=CC(=O)N1CC[C@@H](N2CC(c3cc(C)c4c(c3)Nc3nccc(N5CCOCC5)c3CO4)C2)[C@@H](F)C1] |
| 203 | 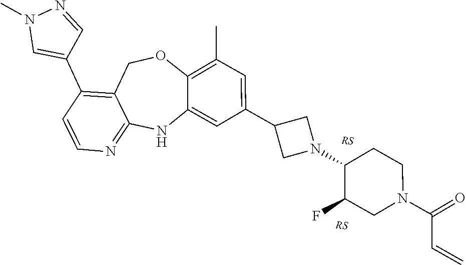 [SMILES: C=CC(=O)N1CC[C@@H](N2CC(c3cc(C)c4c(c3)Nc3nccc(-c5cnn(C)c5)c3CO4)C2)[C@H](F)C1] |
| 204 | 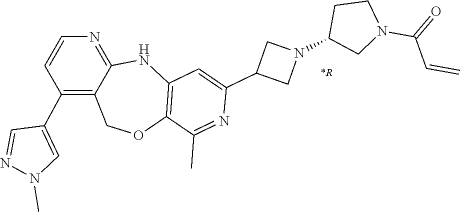 [SMILES: C=CC(=O)N1CC[C@@H](N2CC(c3cc4c(c(C)n3)OCc3c(-c5cnn(C)c5)ccnc3N4)C2)C1] |
| 205 | 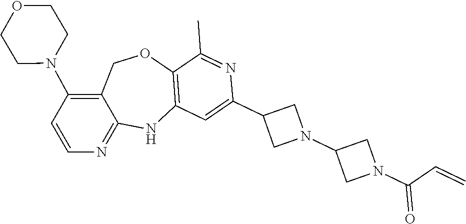 [SMILES: C=CC(=O)N1CC(N2CC(c3cc4c(c(C)n3)OCc3c(N5CCOCC5)ccnc3N4)C2)C1] |
| 206 | 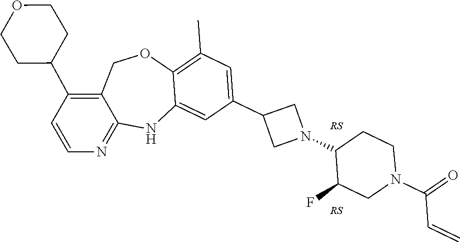 |
| 207 | 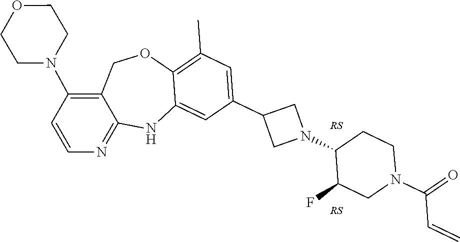 [SMILES: C=CC(=O)N1CC[C@@H](N2CC(c3cc(C)c4c(c3)Nc3nccc(N5CCOCC5)c3CO4)C2)[C@H](F)C1] |
| 208 | 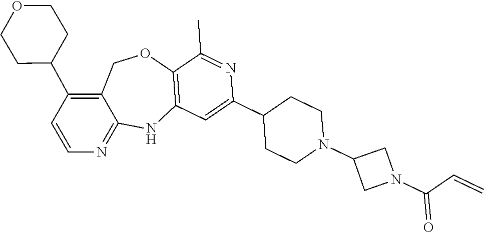 [SMILES: C=CC(=O)N1CC(N2CCC(c3cc4c(c(C)n3)OCc3c(C5CCOCC5)ccnc3N4)CC2)C1] |
| 209 | 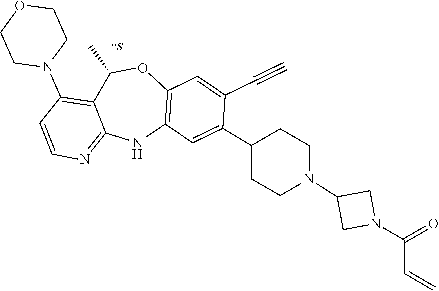 [SMILES: C#Cc1cc2c(cc1C1CCN(C3CN(C(=O)C=C)C3)CC1)Nc1nccc(N3CCOCC3)c1[C@H](C)O2] |
| 210 | 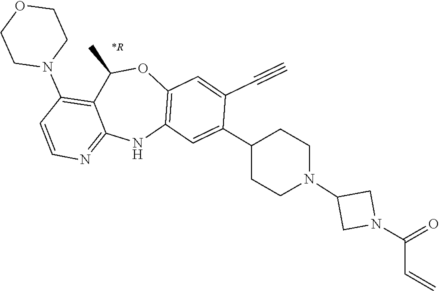 [SMILES: C#Cc1cc2c(cc1C1CCN(C3CN(C(=O)C=C)C3)CC1)Nc1nccc(N3CCOCC3)c1[C@@H](C)O2] |
| 211 | 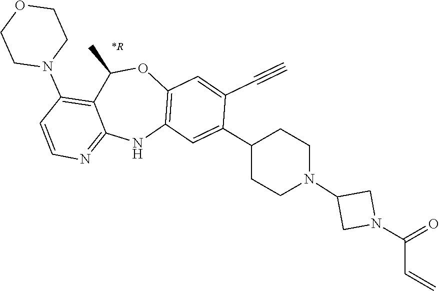 [SMILES: C#Cc1cc2c(cc1C1CCN(C3CN(C(=O)C=C)C3)CC1)Nc1nccc(N3CCOCC3)c1[C@@H](C)O2] |
| 212 | 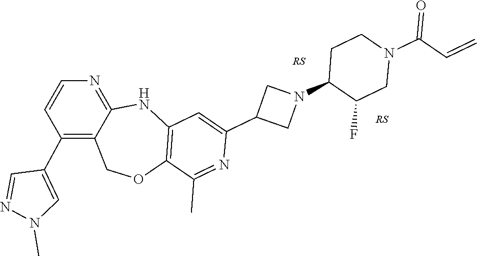 [SMILES: C=CC(=O)N1CC[C@H](N2CC(c3cc4c(c(C)n3)OCc3c(-c5cnn(C)c5)ccnc3N4)C2)[C@@H](F)C1] |
| 213 | 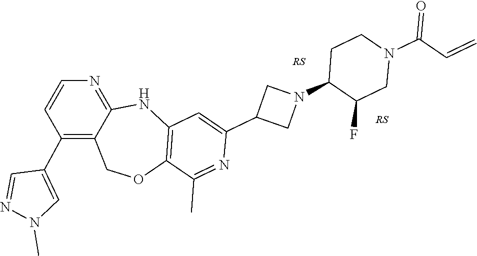 [SMILES: C=CC(=O)N1CC[C@H](N2CC(c3cc4c(c(C)n3)OCc3c(-c5cnn(C)c5)ccnc3N4)C2)[C@H](F)C1] |
| 214 | 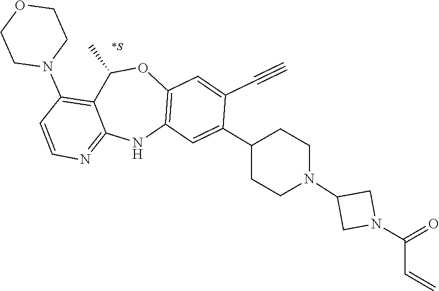 [SMILES: C#Cc1cc2c(cc1C1CCN(C3CN(C(=O)C=C)C3)CC1)Nc1nccc(N3CCOCC3)c1[C@H](C)O2] |
| 215 | 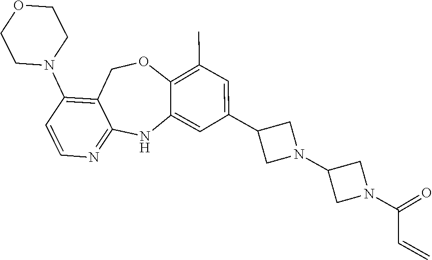 [SMILES: C=CC(=O)N1CC(N2CC(c3cc(C)c4c(c3)Nc3nccc(N5CCOCC5)c3CO4)C2)C1] |
| 216 | 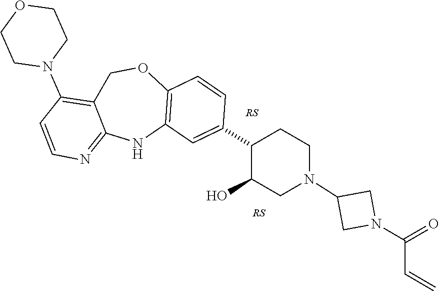 |
| 217 | 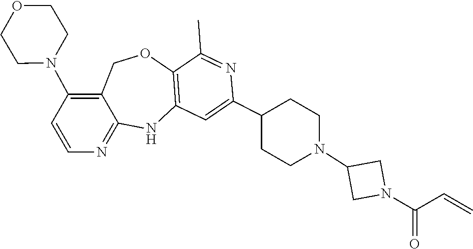 [SMILES: C=CC(=O)N1CC(N2CCC(c3cc4c(c(C)n3)OCc3c(N5CCOCC5)ccnc3N4)CC2)C1] |
| 218 | 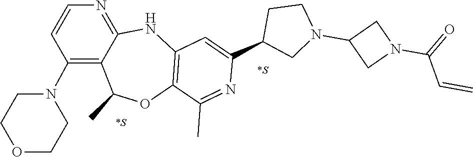 [SMILES: C=CC(=O)N1CC(N2CC[C@H](c3cc4c(c(C)n3)O[C@@H](C)c3c(N5CCOCC5)ccnc3N4)C2)C1] |
| 219 | 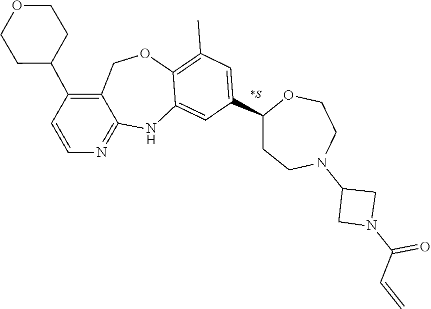 [SMILES: C=CC(=O)N1CC(N2CCO[C@H](c3cc(C)c4c(c3)Nc3nccc(C5CCOCC5)c3CO4)CC2)C1] |
| 220 | 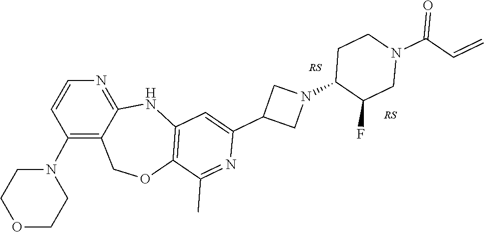 [SMILES: C=CC(=O)N1CC[C@@H](N2CC(c3cc4c(c(C)n3)OCc3c(N5CCOCC5)ccnc3N4)C2)[C@H](F)C1] |
| 221 | 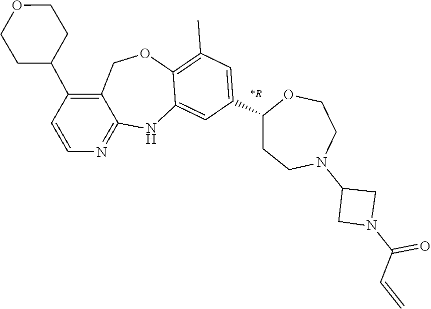 [SMILES: C=CC(=O)N1CC(N2CCO[C@@H](c3cc(C)c4c(c3)Nc3nccc(C5CCOCC5)c3CO4)CC2)C1] |
| 222 | 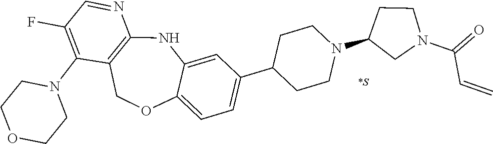 [SMILES: C=CC(=O)N1CC[C@H](N2CCC(c3ccc4c(c3)Nc3ncc(F)c(N5CCOCC5)c3CO4)CC2)C1] |
| 223 | 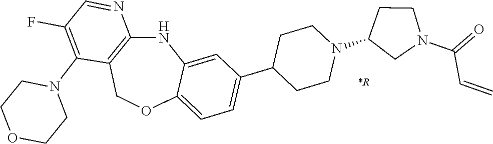 [SMILES: C=CC(=O)N1CC[C@@H](N2CCC(c3ccc4c(c3)Nc3ncc(F)c(N5CCOCC5)c3CO4)CC2)C1] |
| 224 | 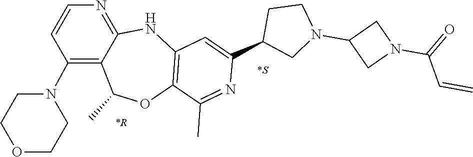 [SMILES: C=CC(=O)N1CC(N2CC[C@H](c3cc4c(c(C)n3)O[C@H](C)c3c(N5CCOCC5)ccnc3N4)C2)C1] |
| 225 | 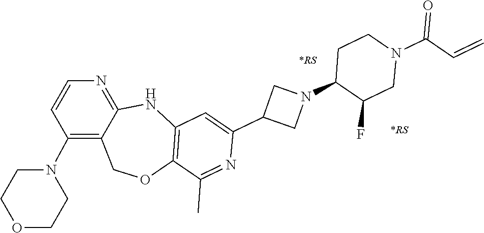 [SMILES: C=CC(=O)N1CC[C@H](N2CC(c3cc4c(c(C)n3)OCc3c(N5CCOCC5)ccnc3N4)C2)[C@H](F)C1] |
| 226 | 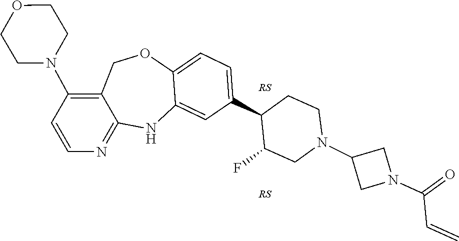 [SMILES: C=CC(=O)N1CC(N2CC[C@H](c3ccc4c(c3)Nc3nccc(N5CCOCC5)c3CO4)[C@@H](F)C2)C1] |
| 227 | 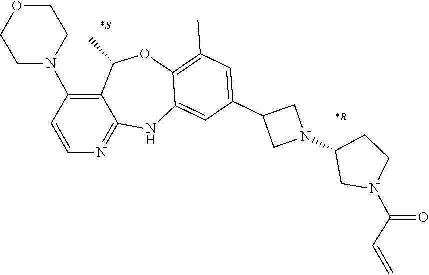 [SMILES: C=CC(=O)N1CC[C@@H](N2CC(c3cc(C)c4c(c3)Nc3nccc(N5CCOCC5)c3[C@H](C)O4)C2)C1] |
| 228 | 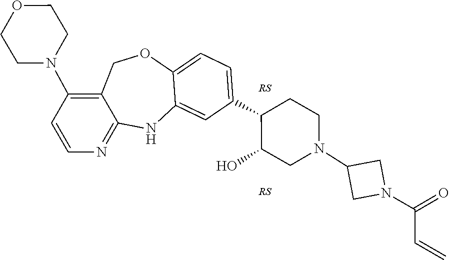 [SMILES: C=CC(=O)N1CC(N2CC[C@@H](c3ccc4c(c3)Nc3nccc(N5CCOCC5)c3CO4)[C@@H](O)C2)C1] |
| 229 | 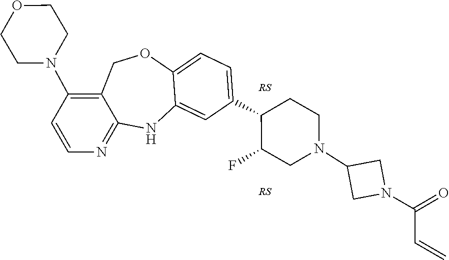 [SMILES: C=CC(=O)N1CC(N2CC[C@@H](c3ccc4c(c3)Nc3nccc(N5CCOCC5)c3CO4)[C@@H](F)C2)C1] |
| 230 | 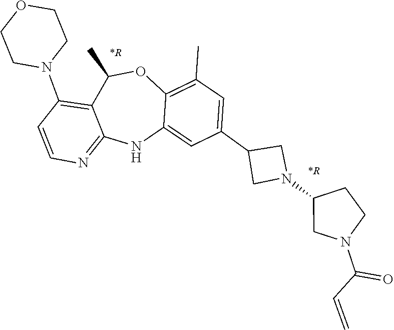 [SMILES: C=CC(=O)N1CC[C@@H](N2CC(c3cc(C)c4c(c3)Nc3nccc(N5CCOCC5)c3[C@@H](C)O4)C2)C1] |
| 231 | 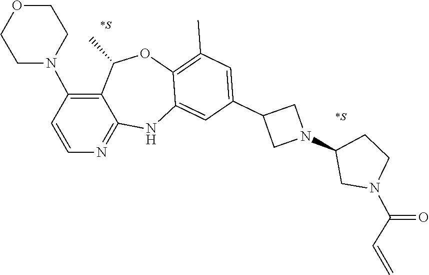 [SMILES: C=CC(=O)N1CC[C@H](N2CC(c3cc(C)c4c(c3)Nc3nccc(N5CCOCC5)c3[C@H](C)O4)C2)C1] |
| 232 | 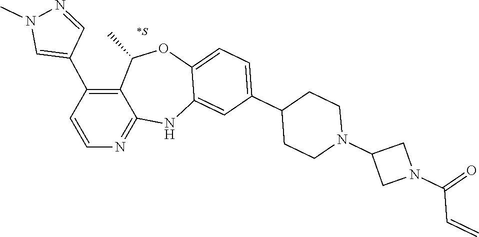 [SMILES: C=CC(=O)N1CC(N2CCC(c3ccc4c(c3)Nc3nccc(-c5cnn(C)c5)c3[C@H](C)O4)CC2)C1] |
| 233 | 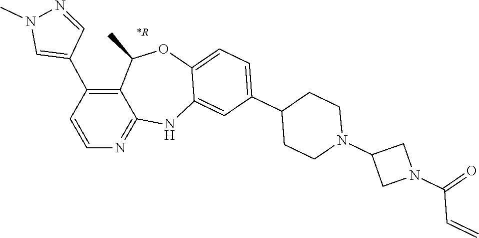 [SMILES: C=CC(=O)N1CC(N2CCC(c3ccc4c(c3)Nc3nccc(-c5cnn(C)c5)c3[C@@H](C)O4)CC2)C1] |
| 234 | 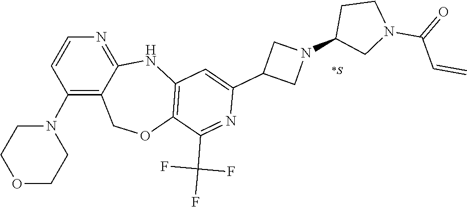 [SMILES: C=CC(=O)N1CC[C@H](N2CC(c3cc4c(c(C(F)(F)F)n3)OCc3c(N5CCOCC5)ccnc3N4)C2)C1] |
| 235 | 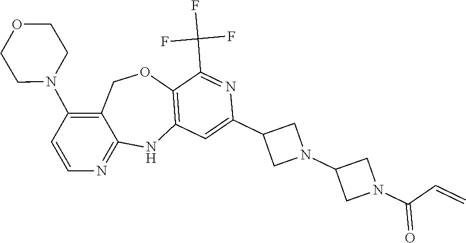 [SMILES: C=CC(=O)N1CC(N2CC(c3cc4c(c(C(F)(F)F)n3)OCc3c(N5CCOCC5)ccnc3N4)C2)C1] |
| 236 | 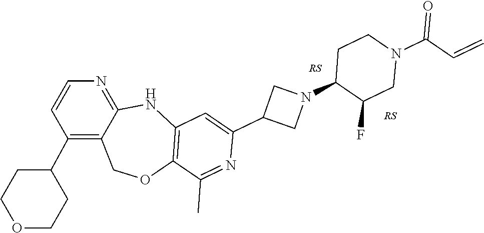 [SMILES: C=CC(=O)N1CC[C@H](N2CC(c3cc4c(c(C)n3)OCc3c(C5CCOCC5)ccnc3N4)C2)[C@H](F)C1] |
| 237 | 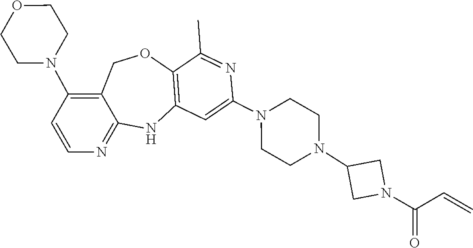 [SMILES: C=CC(=O)N1CC(N2CCN(c3cc4c(c(C)n3)OCc3c(N5CCOCC5)ccnc3N4)CC2)C1] |
| 238 | 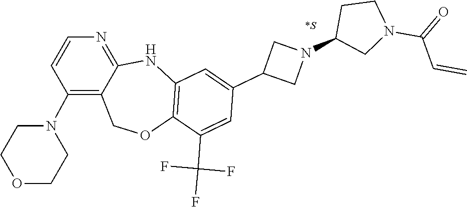 [SMILES: C=CC(=O)N1CC[C@H](N2CC(c3cc4c(c(C(F)(F)F)c3)OCc3c(N5CCOCC5)ccnc3N4)C2)C1] |
| 239 | 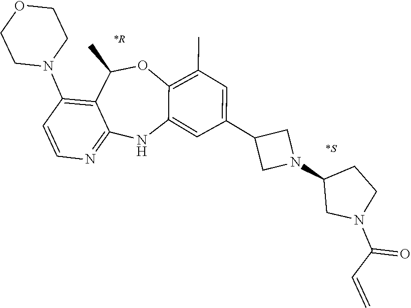 [SMILES: C=CC(=O)N1CC[C@H](N2CC(c3cc(C)c4c(c3)Nc3nccc(N5CCOCC5)c3[C@@H](C)O4)C2)C1] |
| 240 | 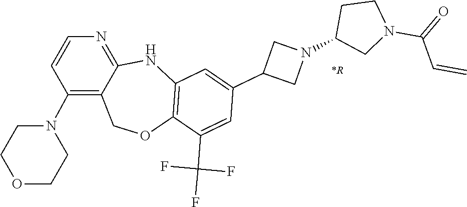 [SMILES: C=CC(=O)N1CC[C@@H](N2CC(c3cc4c(c(C(F)(F)F)c3)OCc3c(N5CCOCC5)ccnc3N4)C2)C1] |
| 241 | 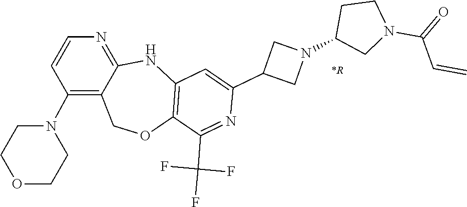 [SMILES: C=CC(=O)N1CC[C@@H](N2CC(c3cc4c(c(C(F)(F)F)n3)OCc3c(N5CCOCC5)ccnc3N4)C2)C1] |
| 242 | 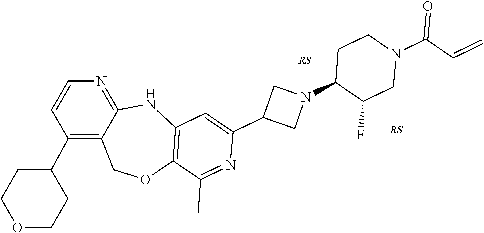 [SMILES: C=CC(=O)N1CC[C@H](N2CC(c3cc4c(c(C)n3)OCc3c(C5CCOCC5)ccnc3N4)C2)[C@@H](F)C1] |
| 243 | 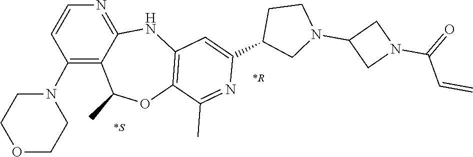 [SMILES: C=CC(=O)N1CC(N2CC[C@@H](c3cc4c(c(C)n3)O[C@@H](C)c3c(N5CCOCC5)ccnc3N4)C2)C1] |
| 244 | 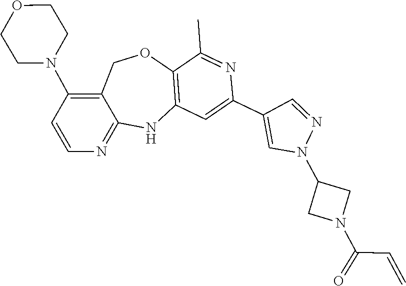 [SMILES: C=CC(=O)N1CC(n2cc(-c3cc4c(c(C)n3)OCc3c(N5CCOCC5)ccnc3N4)cn2)C1] |
| 245 | 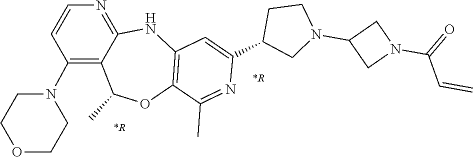 [SMILES: C=CC(=O)N1CC(N2CC[C@@H](c3cc4c(c(C)n3)O[C@H](C)c3c(N5CCOCC5)ccnc3N4)C2)C1] |
| 246 | 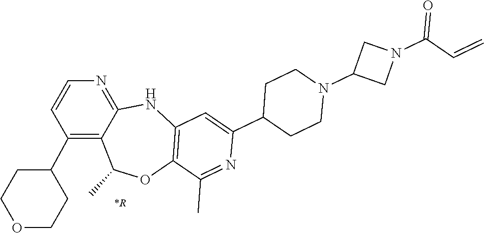 [SMILES: C=CC(=O)N1CC(N2CCC(c3cc4c(c(C)n3)O[C@H](C)c3c(C5CCOCC5)ccnc3N4)CC2)C1] |
| 247 | 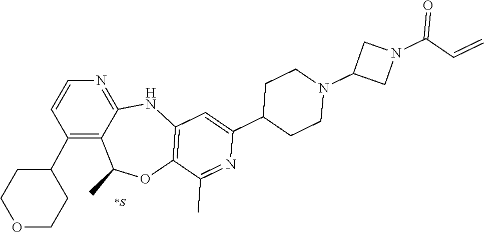 [SMILES: C=CC(=O)N1CC(N2CCC(c3cc4c(c(C)n3)O[C@@H](C)c3c(C5CCOCC5)ccnc3N4)CC2)C1] |
| 248 | 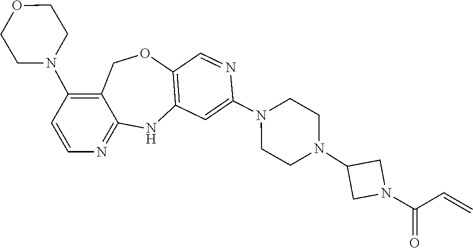 [SMILES: C=CC(=O)N1CC(N2CCN(c3cc4c(cn3)OCc3c(N5CCOCC5)ccnc3N4)CC2)C1] |
| 249 | 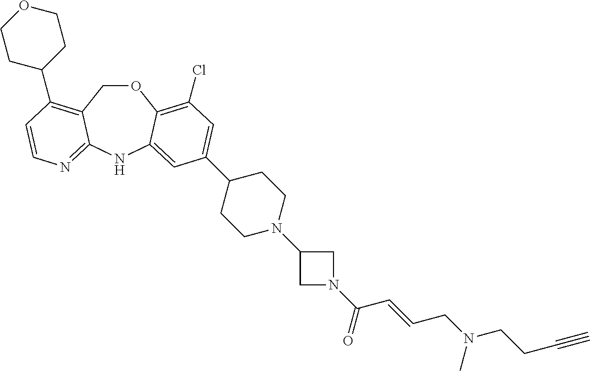 [SMILES: C#CCCN(C)C/C=C/C(=O)N1CC(N2CCC(c3cc(Cl)c4c(c3)Nc3nccc(C5CCOCC5)c3CO4)CC2)C1] |
| 250 | 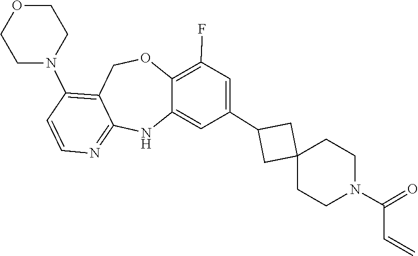 [SMILES: C=CC(=O)N1CCC2(CC1)CC(c1cc(F)c3c(c1)Nc1nccc(N4CCOCC4)c1CO3)C2] |
| 251 | 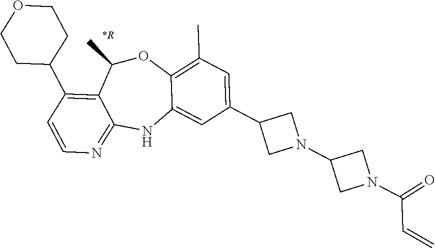 [SMILES: C=CC(=O)N1CC(N2CC(c3cc(C)c4c(c3)Nc3nccc(C5CCOCC5)c3[C@@H](C)O4)C2)C1] |
| 252 | 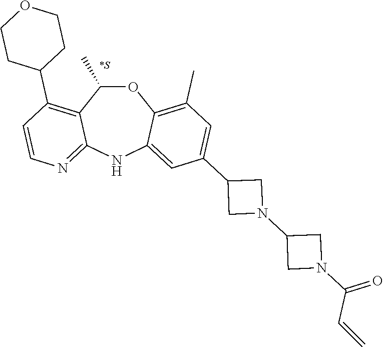 [SMILES: C=CC(=O)N1CC(N2CC(c3cc(C)c4c(c3)Nc3nccc(C5CCOCC5)c3[C@H](C)O4)C2)C1] |
| 253 | 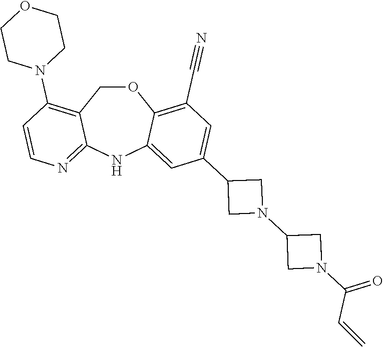 [SMILES: C=CC(=O)N1CC(N2CC(c3cc(C#N)c4c(c3)Nc3nccc(N5CCOCC5)c3CO4)C2)C1] |
| 254 | 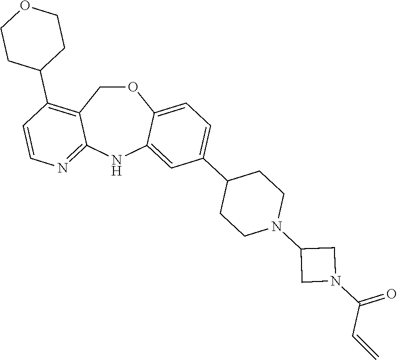 [SMILES: C=CC(=O)N1CC(N2CCC(c3ccc4c(c3)Nc3nccc(C5CCOCC5)c3CO4)CC2)C1] |
| 255 | 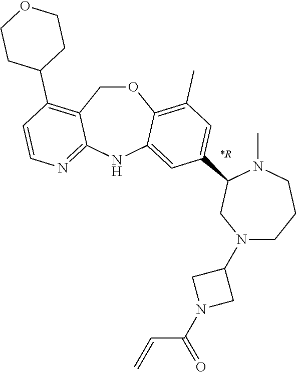 [SMILES: C=CC(=O)N1CC(N2CCCN(C)[C@H](c3cc(C)c4c(c3)Nc3nccc(C5CCOCC5)c3CO4)C2)C1] |
| 256 | 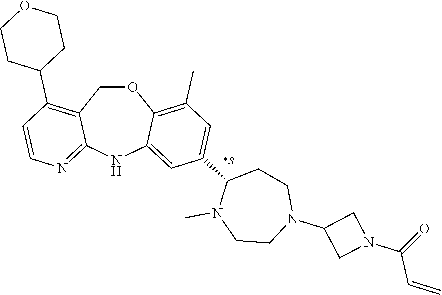 [SMILES: C=CC(=O)N1CC(N2CC[C@@H](c3cc(C)c4c(c3)Nc3nccc(C5CCOCC5)c3CO4)N(C)CC2)C1] |
| 257 | 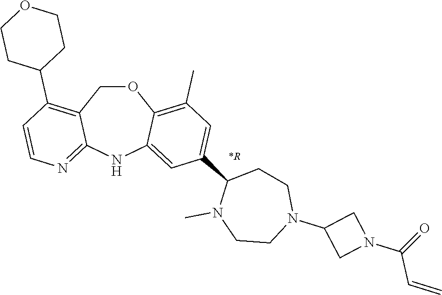 [SMILES: C=CC(=O)N1CC(N2CC[C@H](c3cc(C)c4c(c3)Nc3nccc(C5CCOCC5)c3CO4)N(C)CC2)C1] |
| 258 | 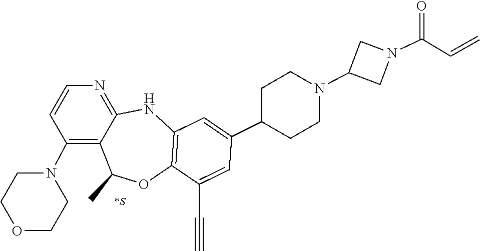 [SMILES: C#Cc1cc(C2CCN(C3CN(C(=O)C=C)C3)CC2)cc2c1O[C@@H](C)c1c(N3CCOCC3)ccnc1N2] |
| 259 | 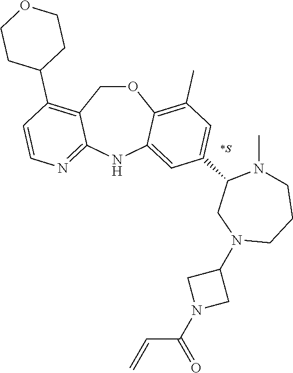 [SMILES: C=CC(=O)N1CC(N2CCCN(C)[C@@H](c3cc(C)c4c(c3)Nc3nccc(C5CCOCC5)c3CO4)C2)C1] |
| 260 | 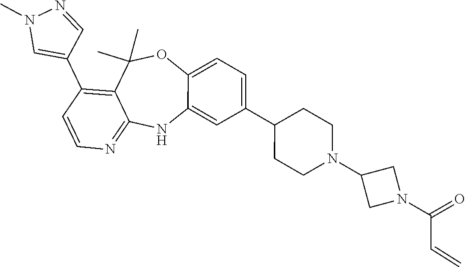 [SMILES: C=CC(=O)N1CC(N2CCC(c3ccc4c(c3)Nc3nccc(-c5cnn(C)c5)c3C(C)(C)O4)CC2)C1] |
| 261 | 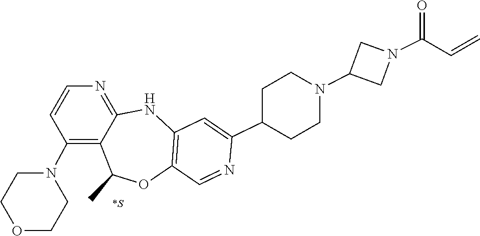 [SMILES: C=CC(=O)N1CC(N2CCC(c3cc4c(cn3)O[C@@H](C)c3c(N5CCOCC5)ccnc3N4)CC2)C1] |
| 262 | 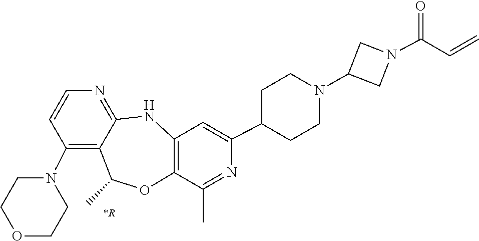 [SMILES: C=CC(=O)N1CC(N2CCC(c3cc4c(c(C)n3)O[C@H](C)c3c(N5CCOCC5)ccnc3N4)CC2)C1] |
| 263 | 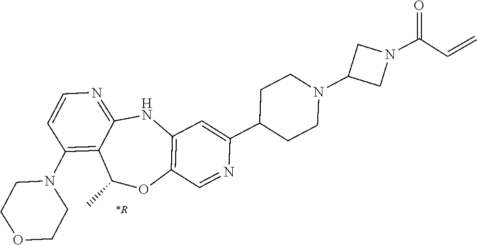 [SMILES: C=CC(=O)N1CC(N2CCC(c3cc4c(cn3)O[C@H](C)c3c(N5CCOCC5)ccnc3N4)CC2)C1] |
| 264 | 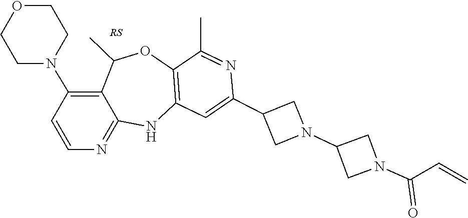 [SMILES: C=CC(=O)N1CC(N2CC(c3cc4c(c(C)n3)OC(C)c3c(N5CCOCC5)ccnc3N4)C2)C1] |
| 265 | 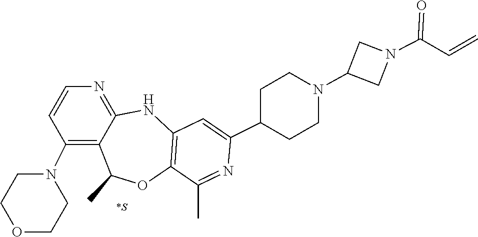 [SMILES: C=CC(=O)N1CC(N2CCC(c3cc4c(c(C)n3)O[C@@H](C)c3c(N5CCOCC5)ccnc3N4)CC2)C1] |
| 266 | 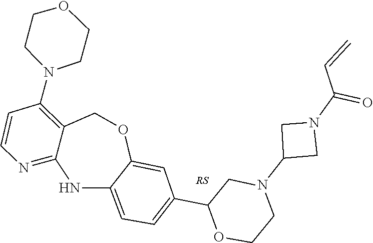 [SMILES: C=CC(=O)N1CC(N2CCOC(c3ccc4c(c3)OCc3c(N5CCOCC5)ccnc3N4)C2)C1] |
| 267 | 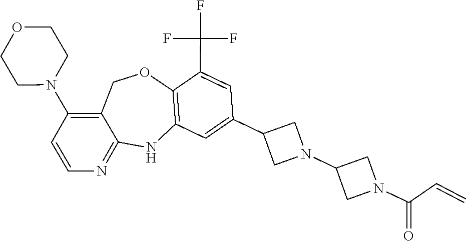 [SMILES: C=CC(=O)N1CC(N2CC(c3cc4c(c(C(F)(F)F)c3)OCc3c(N5CCOCC5)ccnc3N4)C2)C1] |
| 268 | 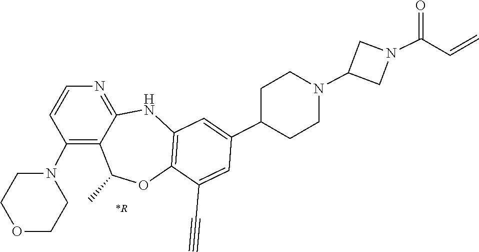 [SMILES: C#Cc1cc(C2CCN(C3CN(C(=O)C=C)C3)CC2)cc2c1O[C@H](C)c1c(N3CCOCC3)ccnc1N2] |
| 269 | 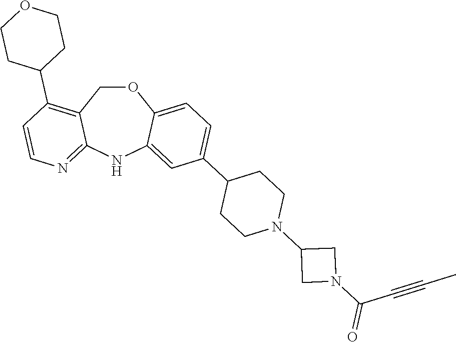 [SMILES: CC#CC(=O)N1CC(N2CCC(c3ccc4c(c3)Nc3nccc(C5CCOCC5)c3CO4)CC2)C1] |
| 270 | 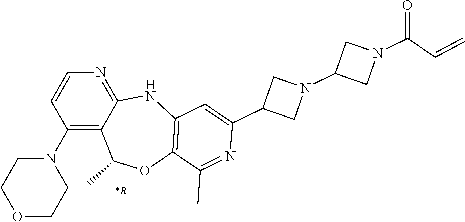 [SMILES: C=CC(=O)N1CC(N2CC(c3cc4c(c(C)n3)O[C@H](C)c3c(N5CCOCC5)ccnc3N4)C2)C1] |
| 271 | 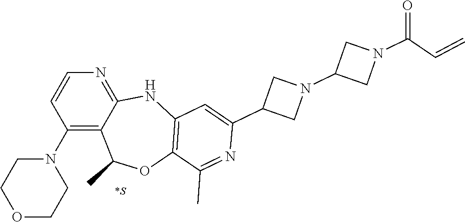 [SMILES: C=CC(=O)N1CC(N2CC(c3cc4c(c(C)n3)O[C@@H](C)c3c(N5CCOCC5)ccnc3N4)C2)C1] |
| 272 | 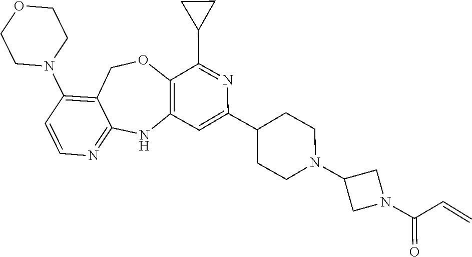 [SMILES: C=CC(=O)N1CC(N2CCC(c3cc4c(c(C5CC5)n3)OCc3c(N5CCOCC5)ccnc3N4)CC2)C1] |
| 273 | 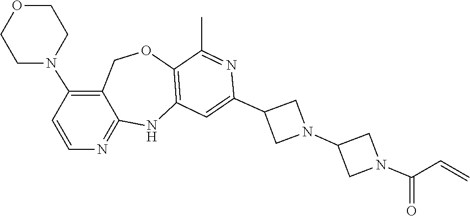 [SMILES: C=CC(=O)N1CC(N2CC(c3cc4c(c(C)n3)OCc3c(N5CCOCC5)ccnc3N4)C2)C1] |
| 274 | 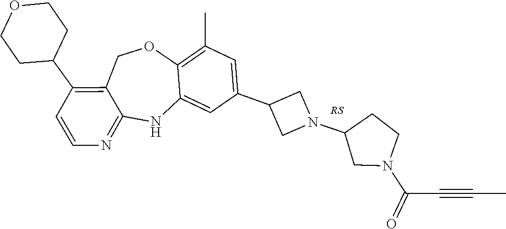 [SMILES: CC#CC(=O)N1CCC(N2CC(c3cc(C)c4c(c3)Nc3nccc(C5CCOCC5)c3CO4)C2)C1] |
| 275 | 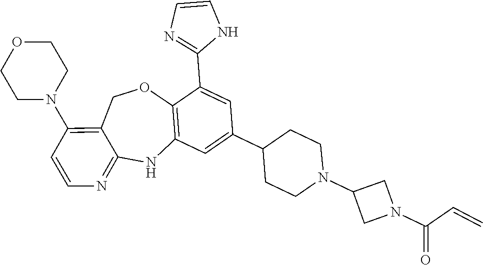 [SMILES: C=CC(=O)N1CC(N2CCC(c3cc4c(c(-c5ncc[nH]5)c3)OCc3c(N5CCOCC5)ccnc3N4)CC2)C1] |
| 276 | 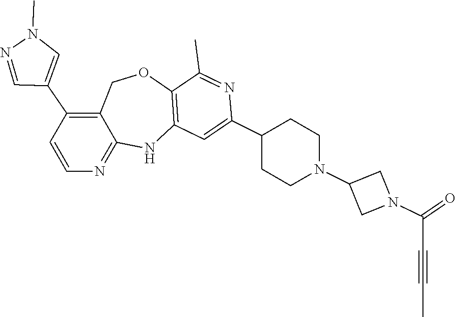 [SMILES: CC#CC(=O)N1CC(N2CCC(c3cc4c(c(C)n3)OCc3c(-c5cnn(C)c5)ccnc3N4)CC2)C1] |
| 277 | 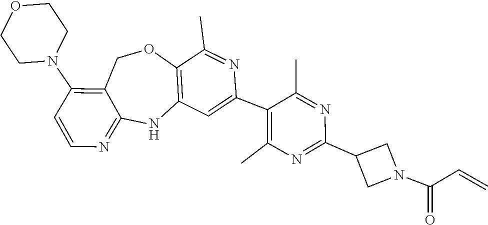 [SMILES: C=CC(=O)N1CC(c2nc(C)c(-c3cc4c(c(C)n3)OCc3c(N5CCOCC5)ccnc3N4)c(C)n2)C1] |
| 278 | 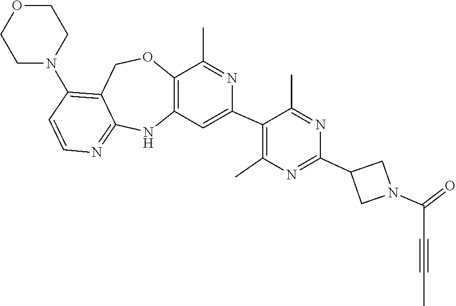 [SMILES: CC#CC(=O)N1CC(c2nc(C)c(-c3cc4c(c(C)n3)OCc3c(N5CCOCC5)ccnc3N4)c(C)n2)C1] |
| 279 | 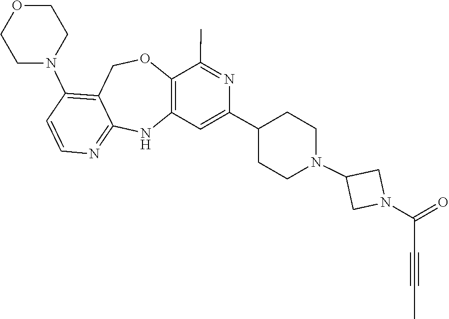 [SMILES: CC#CC(=O)N1CC(N2CCC(c3cc4c(c(C)n3)OCc3c(N5CCOCC5)ccnc3N4)CC2)C1] |
| 280 | 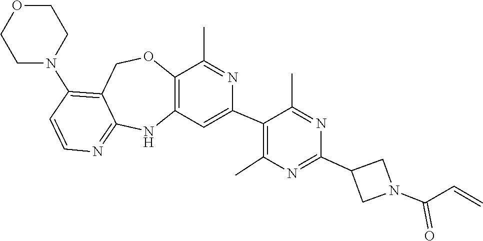 [SMILES: C=CC(=O)N1CC(c2nc(C)c(-c3cc4c(c(C)n3)OCc3c(N5CCOCC5)ccnc3N4)c(C)n2)C1] |
| 281 | 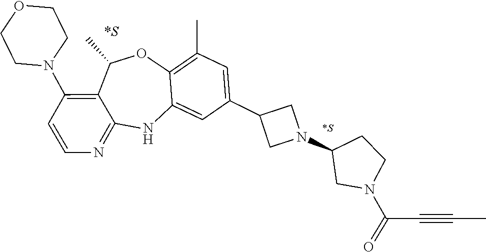 [SMILES: CC#CC(=O)N1CC[C@H](N2CC(c3cc(C)c4c(c3)Nc3nccc(N5CCOCC5)c3[C@H](C)O4)C2)C1] |
| 282 | 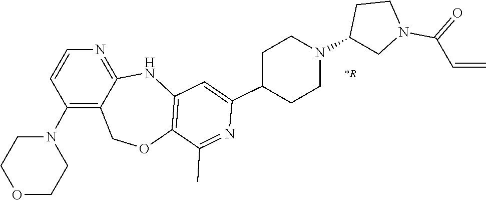 [SMILES: C=CC(=O)N1CC[C@@H](N2CCC(c3cc4c(c(C)n3)OCc3c(N5CCOCC5)ccnc3N4)CC2)C1] |
| 283 | 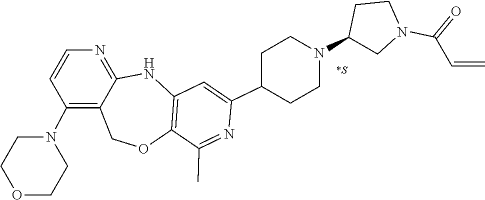 [SMILES: C=CC(=O)N1CC[C@H](N2CCC(c3cc4c(c(C)n3)OCc3c(N5CCOCC5)ccnc3N4)CC2)C1] |
| 284 | 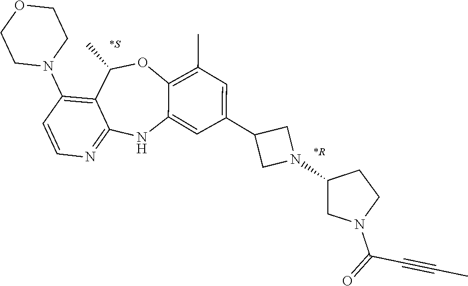 [SMILES: CC#CC(=O)N1CC[C@@H](N2CC(c3cc(C)c4c(c3)Nc3nccc(N5CCOCC5)c3[C@H](C)O4)C2)C1] |
| 285 | 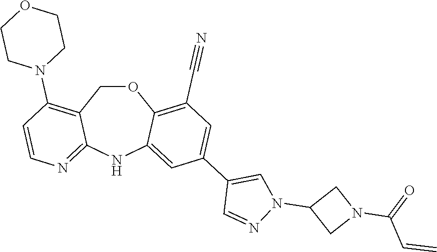 [SMILES: C=CC(=O)N1CC(n2cc(-c3cc(C#N)c4c(c3)Nc3nccc(N5CCOCC5)c3CO4)cn2)C1] |
| 286 | 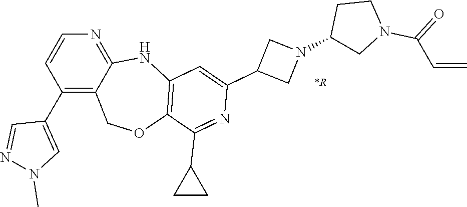 [SMILES: C=CC(=O)N1CC[C@@H](N2CC(c3cc4c(c(C5CC5)n3)OCc3c(-c5cnn(C)c5)ccnc3N4)C2)C1] |
| 287 | 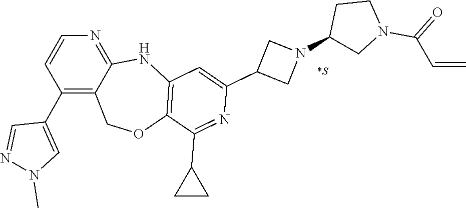 [SMILES: C=CC(=O)N1CC[C@H](N2CC(c3cc4c(c(C5CC5)n3)OCc3c(-c5cnn(C)c5)ccnc3N4)C2)C1] |
| 288 | 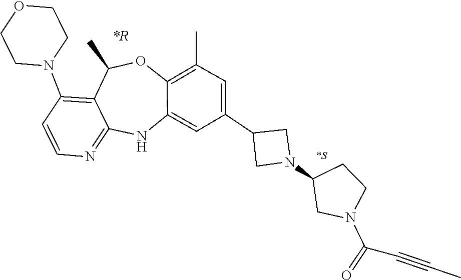 [SMILES: CC#CC(=O)N1CC[C@H](N2CC(c3cc(C)c4c(c3)Nc3nccc(N5CCOCC5)c3[C@@H](C)O4)C2)C1] |
| 289 | 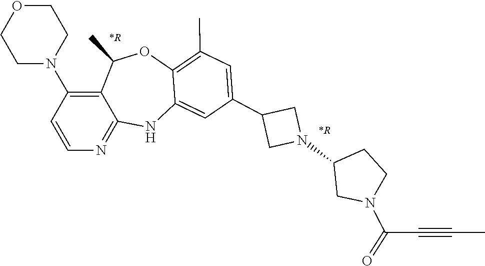 [SMILES: CC#CC(=O)N1CC[C@@H](N2CC(c3cc(C)c4c(c3)Nc3nccc(N5CCOCC5)c3[C@@H](C)O4)C2)C1] |
| 290 | 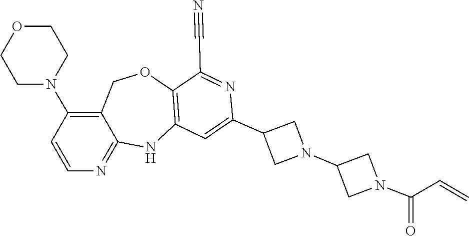 [SMILES: C=CC(=O)N1CC(N2CC(c3cc4c(c(C#N)n3)OCc3c(N5CCOCC5)ccnc3N4)C2)C1] |
| 291 | 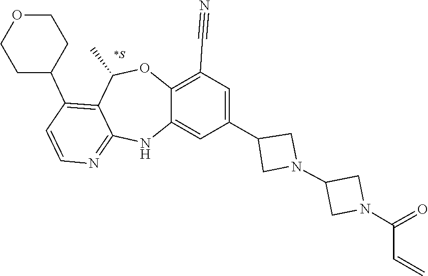 [SMILES: C=CC(=O)N1CC(N2CC(c3cc(C#N)c4c(c3)Nc3nccc(C5CCOCC5)c3[C@H](C)O4)C2)C1] |
| 292 | 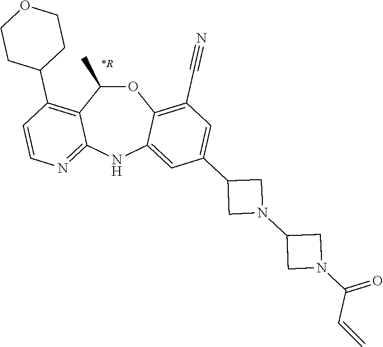 [SMILES: C=CC(=O)N1CC(N2CC(c3cc(C#N)c4c(c3)Nc3nccc(C5CCOCC5)c3[C@@H](C)O4)C2)C1] |
| 293 | 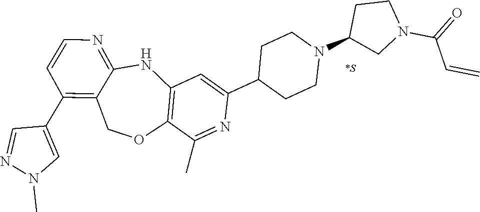 [SMILES: C=CC(=O)N1CC[C@H](N2CCC(c3cc4c(c(C)n3)OCc3c(-c5cnn(C)c5)ccnc3N4)CC2)C1] |
| 294 | 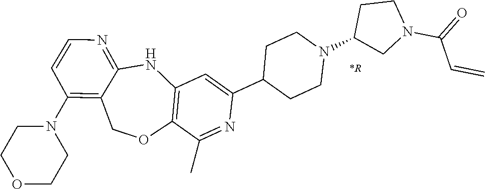 [SMILES: C=CC(=O)N1CC[C@@H](N2CCC(c3cc4c(c(C)n3)OCc3c(N5CCOCC5)ccnc3N4)CC2)C1] |
| 295 | 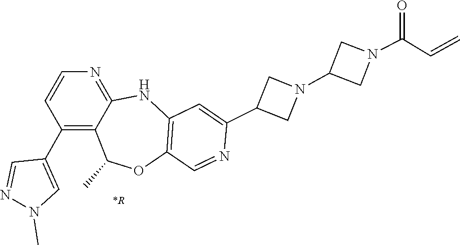 [SMILES: C=CC(=O)N1CC(N2CC(c3cc4c(cn3)O[C@H](C)c3c(-c5cnn(C)c5)ccnc3N4)C2)C1] |
| 296 | 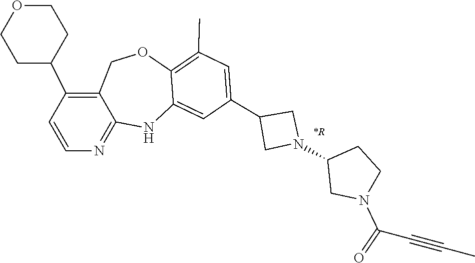 [SMILES: CC#CC(=O)N1CC[C@@H](N2CC(c3cc(C)c4c(c3)Nc3nccc(C5CCOCC5)c3CO4)C2)C1] |
| 297 | 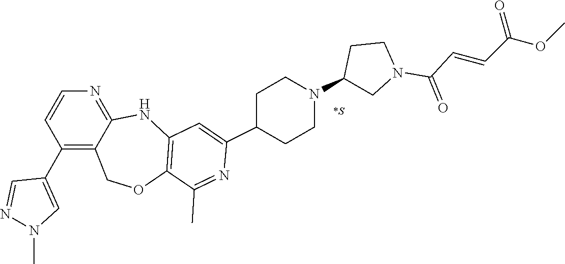 [SMILES: COC(=O)/C=C/C(=O)N1CC[C@H](N2CCC(c3cc4c(c(C)n3)OCc3c(-c5cnn(C)c5)ccnc3N4)CC2)C1] |
| 298 | 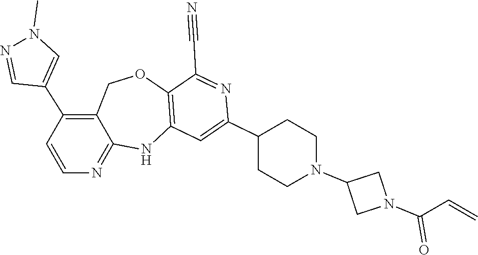 [SMILES: C=CC(=O)N1CC(N2CCC(c3cc4c(c(C#N)n3)OCc3c(-c5cnn(C)c5)ccnc3N4)CC2)C1] |
| 299 | 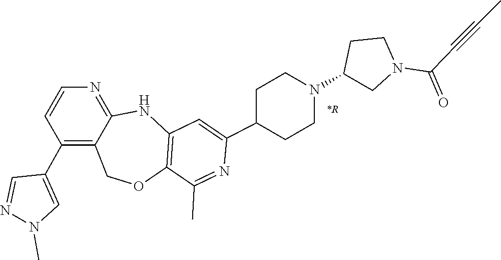 [SMILES: CC#CC(=O)N1CC[C@@H](N2CCC(c3cc4c(c(C)n3)OCc3c(-c5cnn(C)c5)ccnc3N4)CC2)C1] |
| 300 | 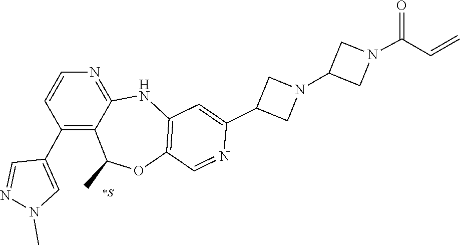 [SMILES: C=CC(=O)N1CC(N2CC(c3cc4c(cn3)O[C@@H](C)c3c(-c5cnn(C)c5)ccnc3N4)C2)C1] |
| 301 | 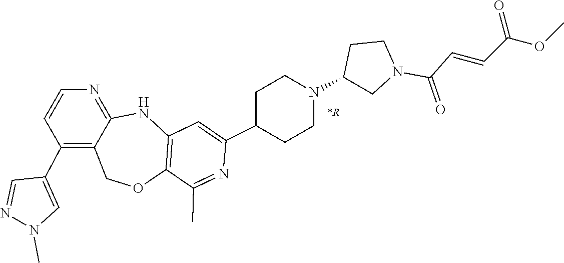 [SMILES: COC(=O)/C=C/C(=O)N1CC[C@@H](N2CCC(c3cc4c(c(C)n3)OCc3c(-c5cnn(C)c5)ccnc3N4)CC2)C1] |
| 302 | 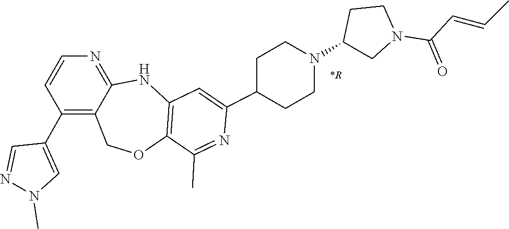 [SMILES: C/C=C/C(=O)N1CC[C@@H](N2CCC(c3cc4c(c(C)n3)OCc3c(-c5cnn(C)c5)ccnc3N4)CC2)C1] |
| 303 | 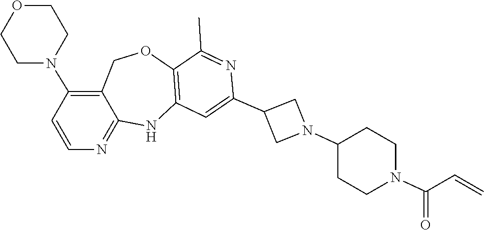 [SMILES: C=CC(=O)N1CCC(N2CC(c3cc4c(c(C)n3)OCc3c(N5CCOCC5)ccnc3N4)C2)CC1] |
| 304 | 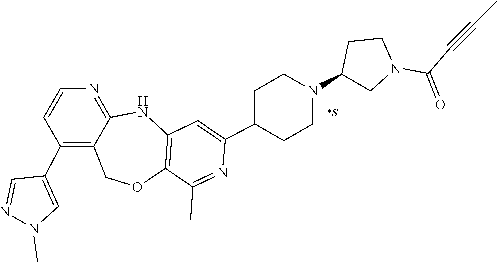 [SMILES: CC#CC(=O)N1CC[C@H](N2CCC(c3cc4c(c(C)n3)OCc3c(-c5cnn(C)c5)ccnc3N4)CC2)C1] |
| 305 | 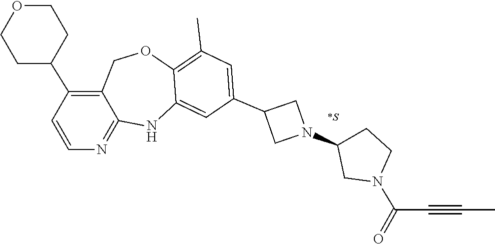 [SMILES: CC#CC(=O)N1CC[C@H](N2CC(c3cc(C)c4c(c3)Nc3nccc(C5CCOCC5)c3CO4)C2)C1] |
| 306 | 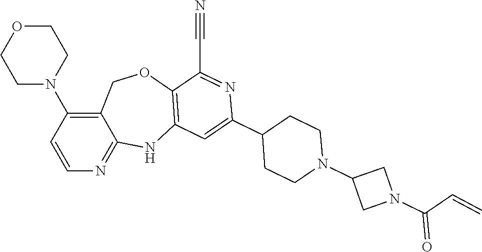 [SMILES: C=CC(=O)N1CC(N2CCC(c3cc4c(c(C#N)n3)OCc3c(N5CCOCC5)ccnc3N4)CC2)C1] |
| 307 | 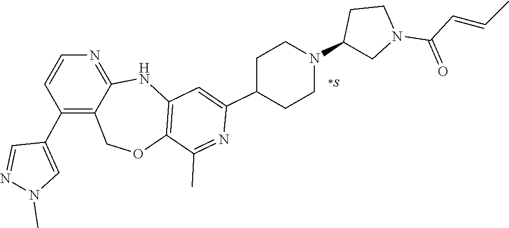 [SMILES: C/C=C/C(=O)N1CC[C@H](N2CCC(c3cc4c(c(C)n3)OCc3c(-c5cnn(C)c5)ccnc3N4)CC2)C1] |
| 308 | 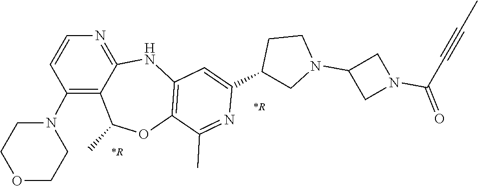 [SMILES: CC#CC(=O)N1CC(N2CC[C@@H](c3cc4c(c(C)n3)O[C@H](C)c3c(N5CCOCC5)ccnc3N4)C2)C1] |
| 309 | 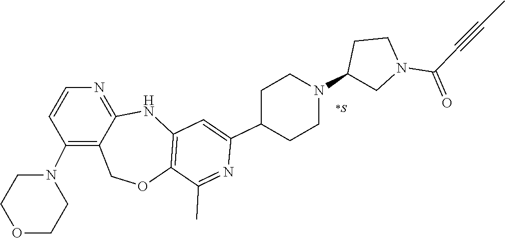 [SMILES: CC#CC(=O)N1CC[C@H](N2CCC(c3cc4c(c(C)n3)OCc3c(N5CCOCC5)ccnc3N4)CC2)C1] |
| 310 | 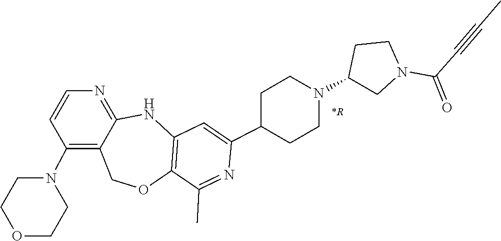 [SMILES: CC#CC(=O)N1CC[C@@H](N2CCC(c3cc4c(c(C)n3)OCc3c(N5CCOCC5)ccnc3N4)CC2)C1] |
| 311 | 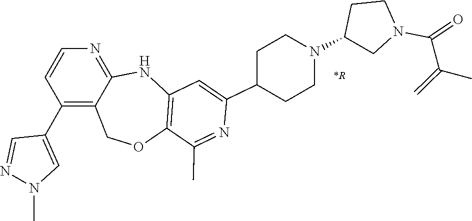 [SMILES: C=C(C)C(=O)N1CC[C@@H](N2CCC(c3cc4c(c(C)n3)OCc3c(-c5cnn(C)c5)ccnc3N4)CC2)C1] |
| 312 | 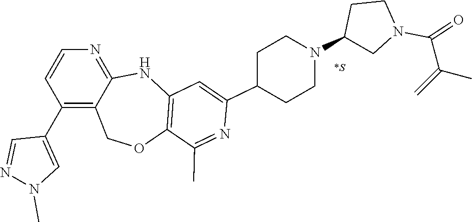 [SMILES: C=C(C)C(=O)N1CC[C@H](N2CCC(c3cc4c(c(C)n3)OCc3c(-c5cnn(C)c5)ccnc3N4)CC2)C1] |
| 313 | 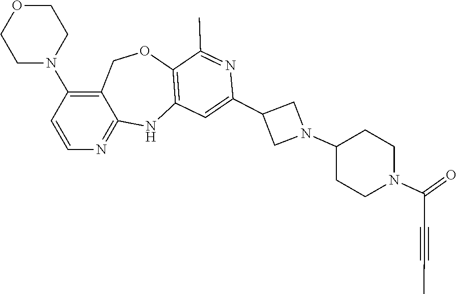 [SMILES: CC#CC(=O)N1CCC(N2CC(c3cc4c(c(C)n3)OCc3c(N5CCOCC5)ccnc3N4)C2)CC1] |
| 314 | 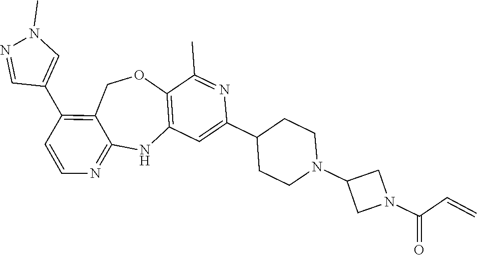 [SMILES: C=CC(=O)N1CC(N2CCC(c3cc4c(c(C)n3)OCc3c(-c5cnn(C)c5)ccnc3N4)CC2)C1] |
| 315 | 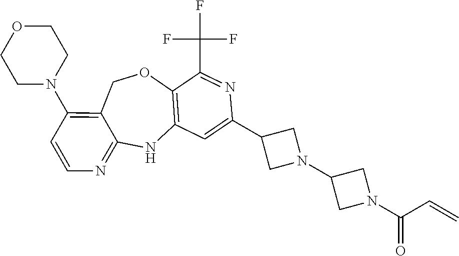 [SMILES: C=CC(=O)N1CC(N2CC(c3cc4c(c(C(F)(F)F)n3)OCc3c(N5CCOCC5)ccnc3N4)C2)C1] |
| 316 | 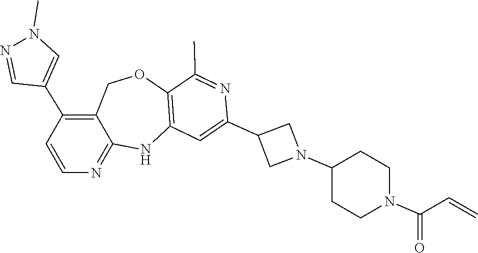 [SMILES: C=CC(=O)N1CCC(N2CC(c3cc4c(c(C)n3)OCc3c(-c5cnn(C)c5)ccnc3N4)C2)CC1] |
| 317 | 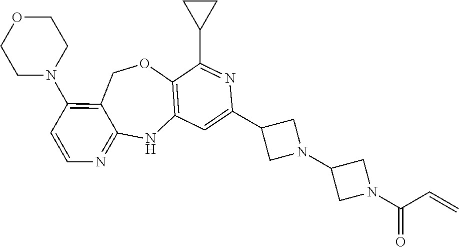 [SMILES: C=CC(=O)N1CC(N2CC(c3cc4c(c(C5CC5)n3)OCc3c(N5CCOCC5)ccnc3N4)C2)C1] |
| 318 | 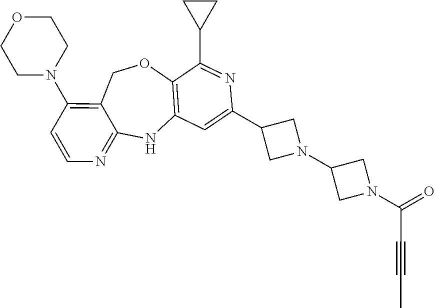 [SMILES: CC#CC(=O)N1CC(N2CC(c3cc4c(c(C5CC5)n3)OCc3c(N5CCOCC5)ccnc3N4)C2)C1] |
| 319 | 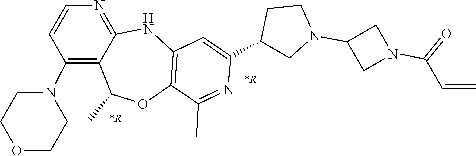 [SMILES: C=CC(=O)N1CC(N2CC[C@@H](c3cc4c(c(C)n3)O[C@H](C)c3c(N5CCOCC5)ccnc3N4)C2)C1] |
| 320 | 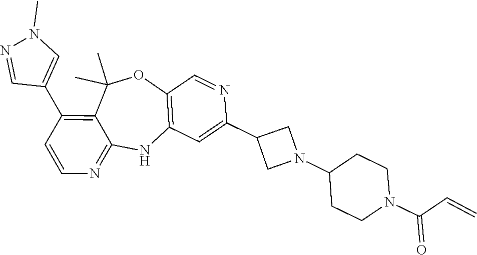 [SMILES: C=CC(=O)N1CCC(N2CC(c3cc4c(cn3)OC(C)(C)c3c(-c5cnn(C)c5)ccnc3N4)C2)CC1] |
| 321 | 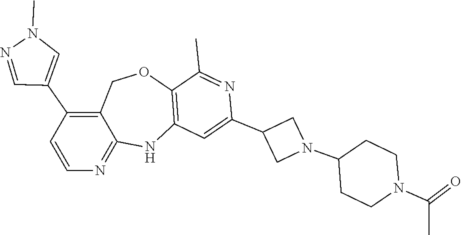 [SMILES: CC(=O)N1CCC(N2CC(c3cc4c(c(C)n3)OCc3c(-c5cnn(C)c5)ccnc3N4)C2)CC1] |
| 322 | 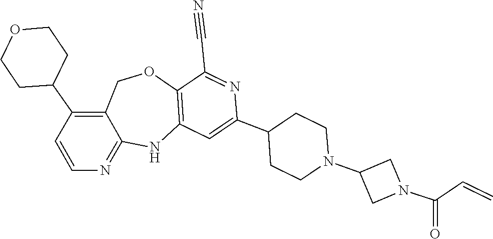 [SMILES: C=CC(=O)N1CC(N2CCC(c3cc4c(c(C#N)n3)OCc3c(C5CCOCC5)ccnc3N4)CC2)C1] |
| 323 | 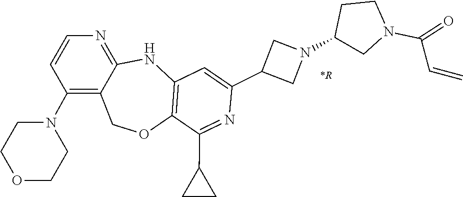 [SMILES: C=CC(=O)N1CC[C@@H](N2CC(c3cc4c(c(C5CC5)n3)OCc3c(N5CCOCC5)ccnc3N4)C2)C1] |
| 324 | 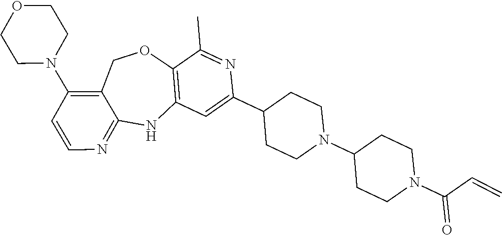 [SMILES: C=CC(=O)N1CCC(N2CCC(c3cc4c(c(C)n3)OCc3c(N5CCOCC5)ccnc3N4)CC2)CC1] |
| 325 | 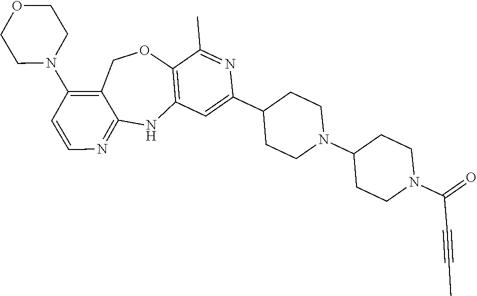 [SMILES: CC#CC(=O)N1CCC(N2CCC(c3cc4c(c(C)n3)OCc3c(N5CCOCC5)ccnc3N4)CC2)CC1] |
| 326 | 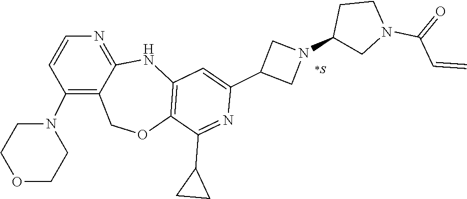 [SMILES: C=CC(=O)N1CC[C@H](N2CC(c3cc4c(c(C5CC5)n3)OCc3c(N5CCOCC5)ccnc3N4)C2)C1] |
| 327 | 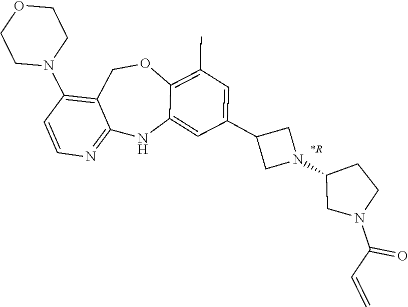 [SMILES: C=CC(=O)N1CC[C@@H](N2CC(c3cc(C)c4c(c3)Nc3nccc(N5CCOCC5)c3CO4)C2)C1] |
| 328 | 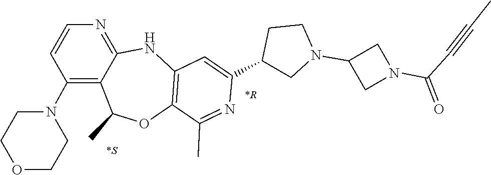 [SMILES: CC#CC(=O)N1CC(N2CC[C@@H](c3cc4c(c(C)n3)O[C@@H](C)c3c(N5CCOCC5)ccnc3N4)C2)C1] |
| 329 | 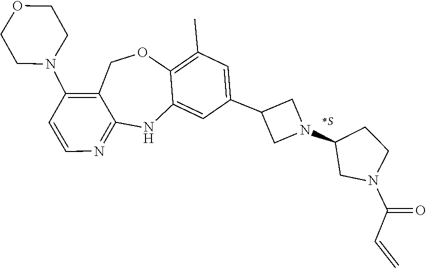 [SMILES: C=CC(=O)N1CC[C@H](N2CC(c3cc(C)c4c(c3)Nc3nccc(N5CCOCC5)c3CO4)C2)C1] |
| 330 | 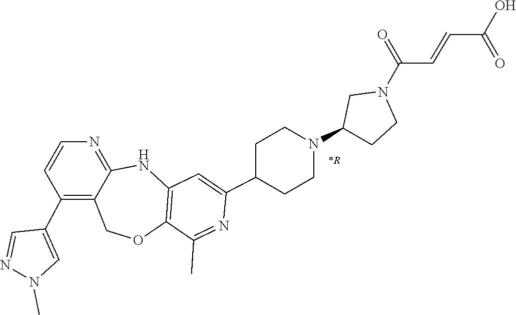 [SMILES: Cc1nc(C2CCN([C@@H]3CCN(C(=O)/C=C/C(=O)O)C3)CC2)cc2c1OCc1c(-c3cnn(C)c3)ccnc1N2] |
| 331 | 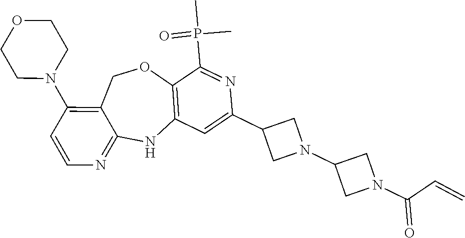 [SMILES: C=CC(=O)N1CC(N2CC(c3cc4c(c(P(C)(C)=O)n3)OCc3c(N5CCOCC5)ccnc3N4)C2)C1] |
| 332 | 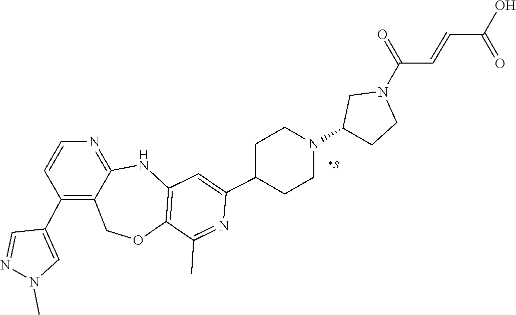 [SMILES: Cc1nc(C2CCN([C@H]3CCN(C(=O)/C=C/C(=O)O)C3)CC2)cc2c1OCc1c(-c3cnn(C)c3)ccnc1N2] |
| 333 | 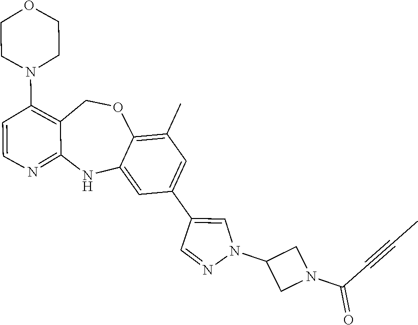 [SMILES: CC#CC(=O)N1CC(n2cc(-c3cc(C)c4c(c3)Nc3nccc(N5CCOCC5)c3CO4)cn2)C1] |
| 334 | 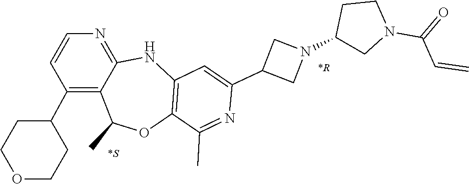 [SMILES: C=CC(=O)N1CC[C@@H](N2CC(c3cc4c(c(C)n3)O[C@@H](C)c3c(C5CCOCC5)ccnc3N4)C2)C1] |
| 335 | 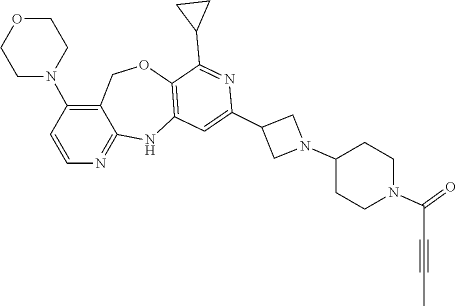 [SMILES: CC#CC(=O)N1CCC(N2CC(c3cc4c(c(C5CC5)n3)OCc3c(N5CCOCC5)ccnc3N4)C2)CC1] |
| 336 | 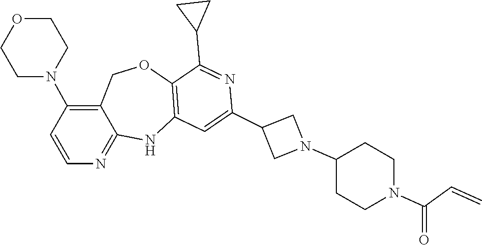 [SMILES: C=CC(=O)N1CCC(N2CC(c3cc4c(c(C5CC5)n3)OCc3c(N5CCOCC5)ccnc3N4)C2)CC1] |
| 337 | 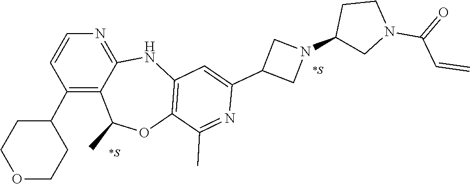 [SMILES: C=CC(=O)N1CC[C@H](N2CC(c3cc4c(c(C)n3)O[C@@H](C)c3c(C5CCOCC5)ccnc3N4)C2)C1] |
| 338 | 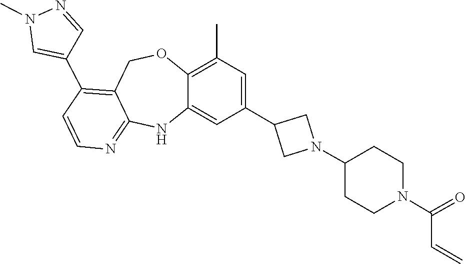 [SMILES: C=CC(=O)N1CCC(N2CC(c3cc(C)c4c(c3)Nc3nccc(-c5cnn(C)c5)c3CO4)C2)CC1] |
| 339 | 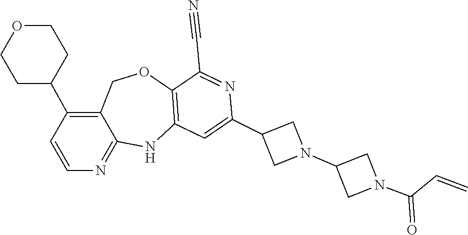 [SMILES: C=CC(=O)N1CC(N2CC(c3cc4c(c(C#N)n3)OCc3c(C5CCOCC5)ccnc3N4)C2)C1] |
| 340 | 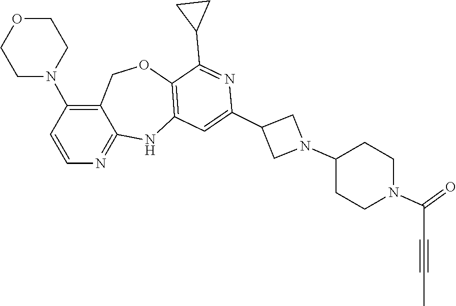 [SMILES: CC#CC(=O)N1CCC(N2CC(c3cc4c(c(C5CC5)n3)OCc3c(N5CCOCC5)ccnc3N4)C2)CC1] |
| 341 | 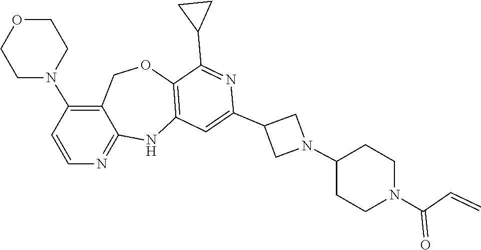 [SMILES: C=CC(=O)N1CCC(N2CC(c3cc4c(c(C5CC5)n3)OCc3c(N5CCOCC5)ccnc3N4)C2)CC1] |
| 342 | 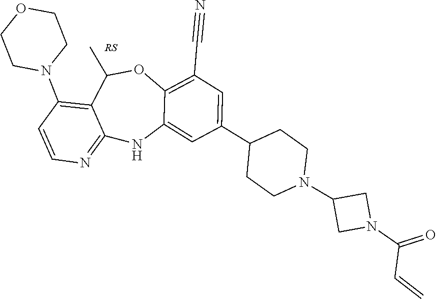 [SMILES: C=CC(=O)N1CC(N2CCC(c3cc(C#N)c4c(c3)Nc3nccc(N5CCOCC5)c3C(C)O4)CC2)C1] |
| 343 | 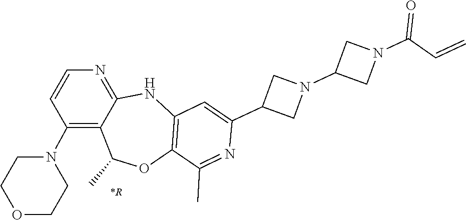 [SMILES: C=CC(=O)N1CC(N2CC(c3cc4c(c(C)n3)O[C@H](C)c3c(N5CCOCC5)ccnc3N4)C2)C1] |
| 344 | 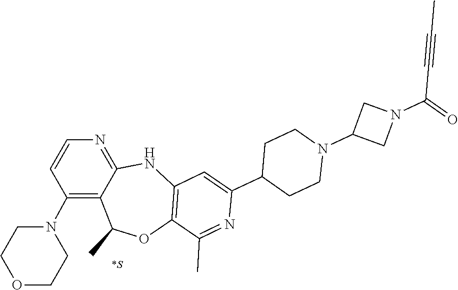 [SMILES: CC#CC(=O)N1CC(N2CCC(c3cc4c(c(C)n3)O[C@@H](C)c3c(N5CCOCC5)ccnc3N4)CC2)C1] |
| 345 | 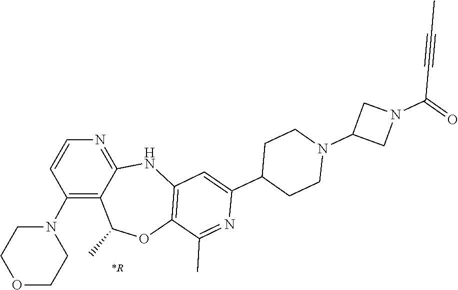 [SMILES: CC#CC(=O)N1CC(N2CCC(c3cc4c(c(C)n3)O[C@H](C)c3c(N5CCOCC5)ccnc3N4)CC2)C1] |
| 346 | 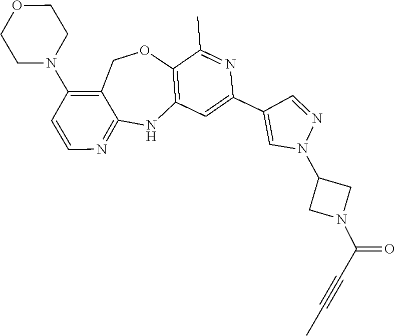 [SMILES: CC#CC(=O)N1CC(n2cc(-c3cc4c(c(C)n3)OCc3c(N5CCOCC5)ccnc3N4)cn2)C1] |
| 347 | 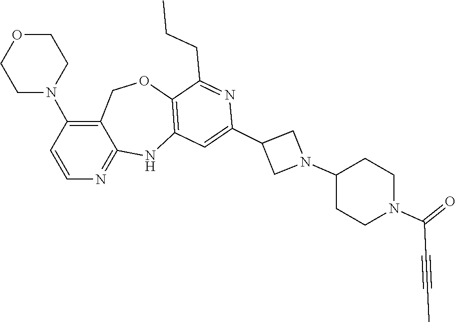 [SMILES: CC#CC(=O)N1CCC(N2CC(c3cc4c(c(CCC)n3)OCc3c(N5CCOCC5)ccnc3N4)C2)CC1] |
| 348 | 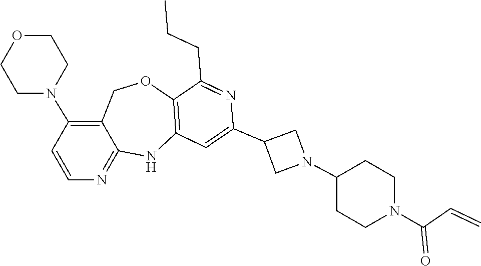 [SMILES: C=CC(=O)N1CCC(N2CC(c3cc4c(c(CCC)n3)OCc3c(N5CCOCC5)ccnc3N4)C2)CC1] |
| 349 | 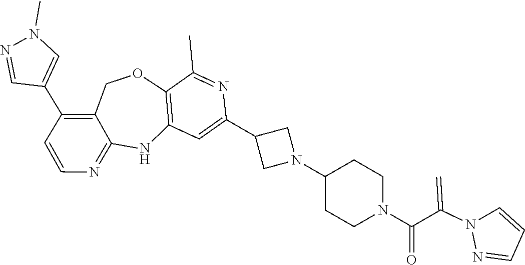 [SMILES: C=C(C(=O)N1CCC(N2CC(c3cc4c(c(C)n3)OCc3c(-c5cnn(C)c5)ccnc3N4)C2)CC1)n1cccn1] |
| 350 | 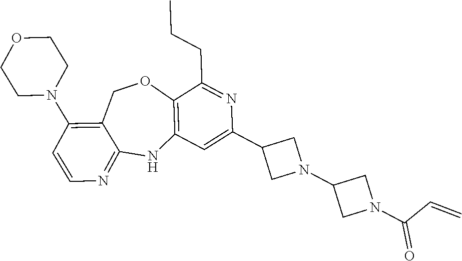 [SMILES: C=CC(=O)N1CC(N2CC(c3cc4c(c(CCC)n3)OCc3c(N5CCOCC5)ccnc3N4)C2)C1] |
| 351 | 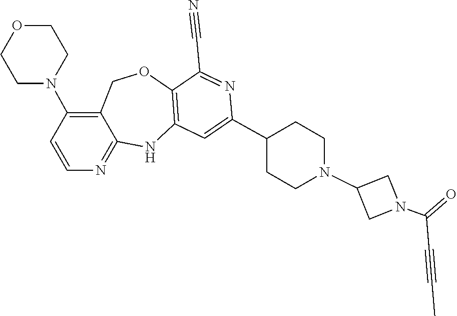 [SMILES: CC#CC(=O)N1CC(N2CCC(c3cc4c(c(C#N)n3)OCc3c(N5CCOCC5)ccnc3N4)CC2)C1] |
| 352 | 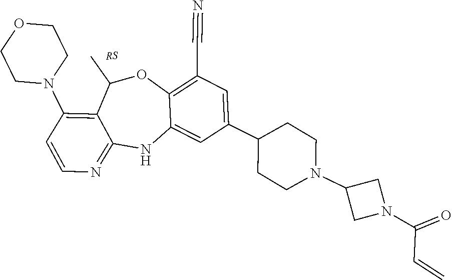 [SMILES: C=CC(=O)N1CC(N2CCC(c3cc(C#N)c4c(c3)Nc3nccc(N5CCOCC5)c3C(C)O4)CC2)C1] |
| 353 | 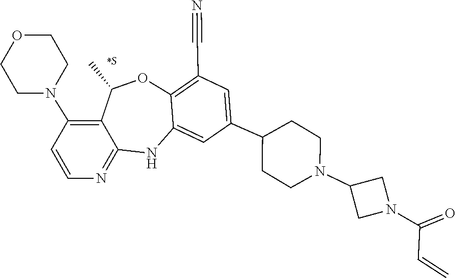 [SMILES: C=CC(=O)N1CC(N2CCC(c3cc(C#N)c4c(c3)Nc3nccc(N5CCOCC5)c3[C@H](C)O4)CC2)C1] |
| 354 | 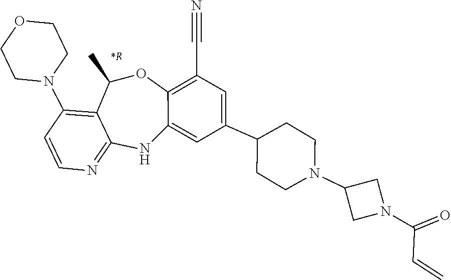 [SMILES: C=CC(=O)N1CC(N2CCC(c3cc(C#N)c4c(c3)Nc3nccc(N5CCOCC5)c3[C@@H](C)O4)CC2)C1] |
| 355 | 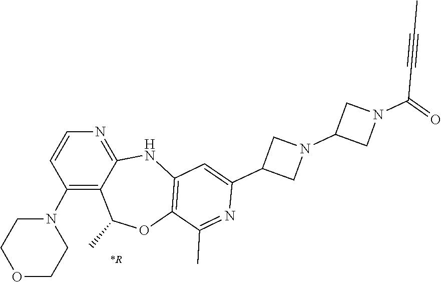 [SMILES: CC#CC(=O)N1CC(N2CC(c3cc4c(c(C)n3)O[C@H](C)c3c(N5CCOCC5)ccnc3N4)C2)C1] |
| 356 | 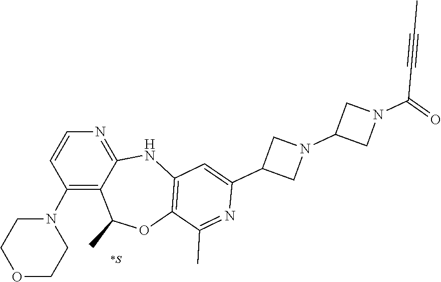 [SMILES: CC#CC(=O)N1CC(N2CC(c3cc4c(c(C)n3)O[C@@H](C)c3c(N5CCOCC5)ccnc3N4)C2)C1] |
| 357 | 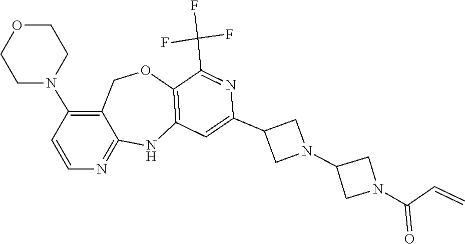 [SMILES: C=CC(=O)N1CC(N2CC(c3cc4c(c(C(F)(F)F)n3)OCc3c(N5CCOCC5)ccnc3N4)C2)C1] |
| 358 | 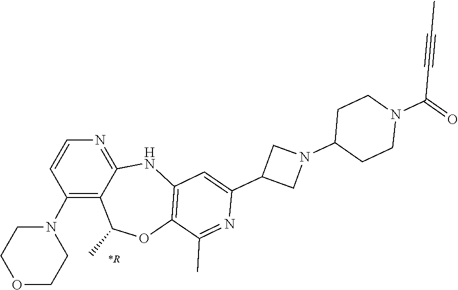 [SMILES: CC#CC(=O)N1CCC(N2CC(c3cc4c(c(C)n3)O[C@H](C)c3c(N5CCOCC5)ccnc3N4)C2)CC1] |
| 359 | 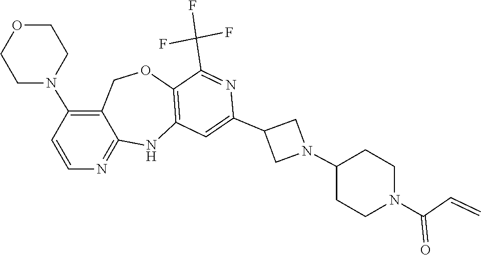 [SMILES: C=CC(=O)N1CCC(N2CC(c3cc4c(c(C(F)(F)F)n3)OCc3c(N5CCOCC5)ccnc3N4)C2)CC1] |
| 360 | 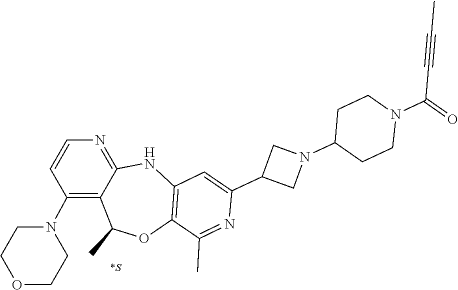 [SMILES: CC#CC(=O)N1CCC(N2CC(c3cc4c(c(C)n3)O[C@@H](C)c3c(N5CCOCC5)ccnc3N4)C2)CC1] |
| 361 | 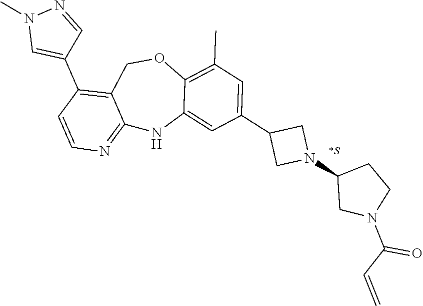 [SMILES: C=CC(=O)N1CC[C@H](N2CC(c3cc(C)c4c(c3)Nc3nccc(-c5cnn(C)c5)c3CO4)C2)C1] |
| 362 | 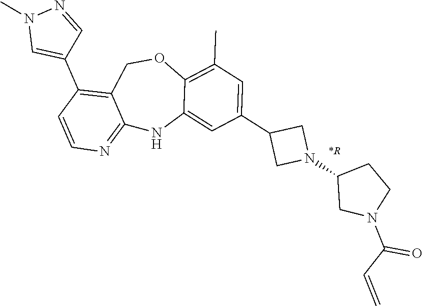 [SMILES: C=CC(=O)N1CC[C@@H](N2CC(c3cc(C)c4c(c3)Nc3nccc(-c5cnn(C)c5)c3CO4)C2)C1] |
| 363 | 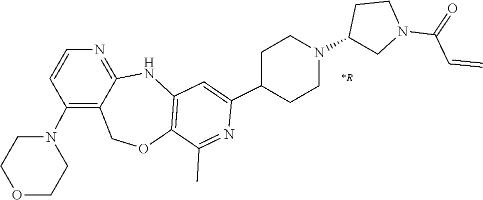 [SMILES: C=CC(=O)N1CC[C@@H](N2CCC(c3cc4c(c(C)n3)OCc3c(N5CCOCC5)ccnc3N4)CC2)C1] |
| 364 | 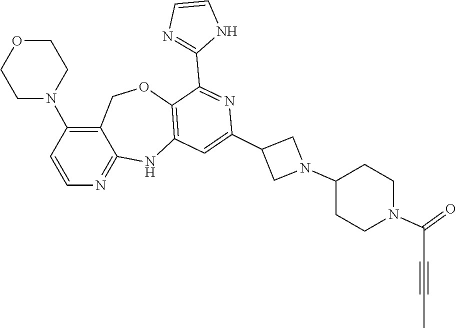 [SMILES: CC#CC(=O)N1CCC(N2CC(c3cc4c(c(-c5ncc[nH]5)n3)OCc3c(N5CCOCC5)ccnc3N4)C2)CC1] |
| 365 | 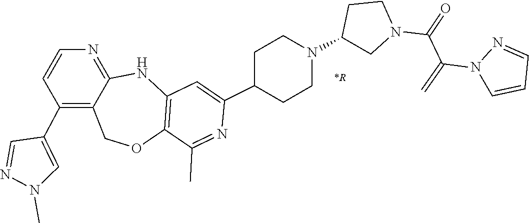 [SMILES: C=C(C(=O)N1CC[C@@H](N2CCC(c3cc4c(c(C)n3)OCc3c(-c5cnn(C)c5)ccnc3N4)CC2)C1)n1cccn1] |
| 366 | 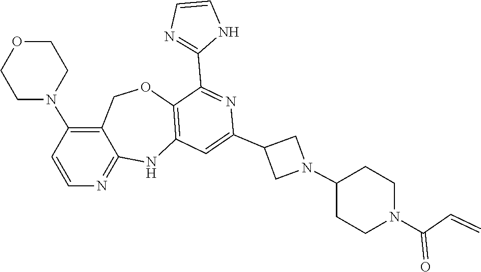 [SMILES: C=CC(=O)N1CCC(N2CC(c3cc4c(c(-c5ncc[nH]5)n3)OCc3c(N5CCOCC5)ccnc3N4)C2)CC1] |
| 367 | 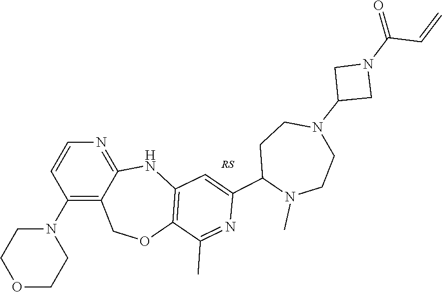 [SMILES: C=CC(=O)N1CC(N2CCC(c3cc4c(c(C)n3)OCc3c(N5CCOCC5)ccnc3N4)N(C)CC2)C1] |
| 368 | 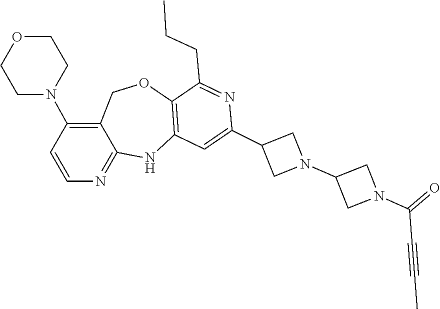 [SMILES: CC#CC(=O)N1CC(N2CC(c3cc4c(c(CCC)n3)OCc3c(N5CCOCC5)ccnc3N4)C2)C1] |
| 369 | 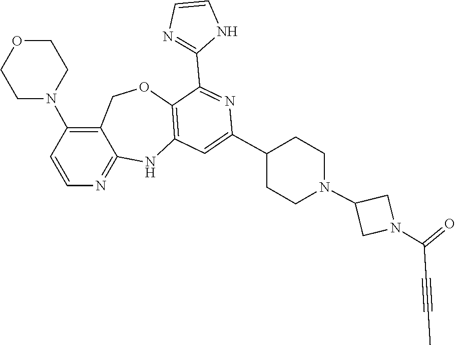 [SMILES: CC#CC(=O)N1CC(N2CCC(c3cc4c(c(-c5ncc[nH]5)n3)OCc3c(N5CCOCC5)ccnc3N4)CC2)C1] |
| 370 | 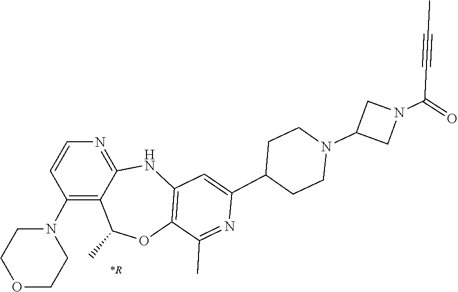 [SMILES: CC#CC(=O)N1CC(N2CCC(c3cc4c(c(C)n3)O[C@H](C)c3c(N5CCOCC5)ccnc3N4)CC2)C1] |
| 371 | 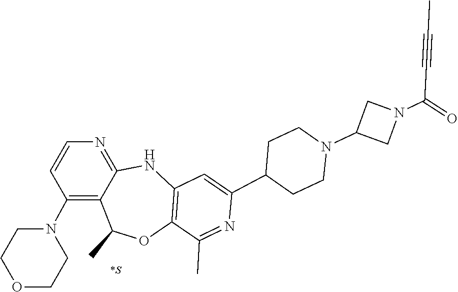 [SMILES: CC#CC(=O)N1CC(N2CCC(c3cc4c(c(C)n3)O[C@@H](C)c3c(N5CCOCC5)ccnc3N4)CC2)C1] |
| 372 | 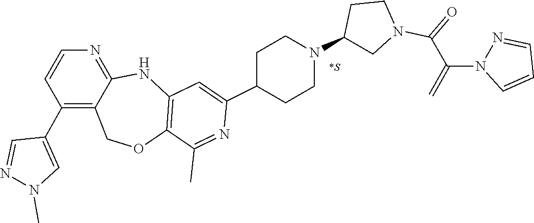 [SMILES: C=C(C(=O)N1CC[C@H](N2CCC(c3cc4c(c(C)n3)OCc3c(-c5cnn(C)c5)ccnc3N4)CC2)C1)n1cccn1] |
| 373 | 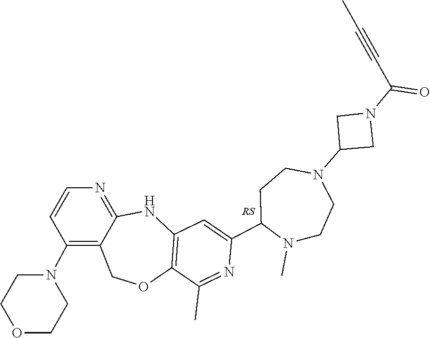 [SMILES: CC#CC(=O)N1CC(N2CCC(c3cc4c(c(C)n3)OCc3c(N5CCOCC5)ccnc3N4)N(C)CC2)C1] |
| 374 | 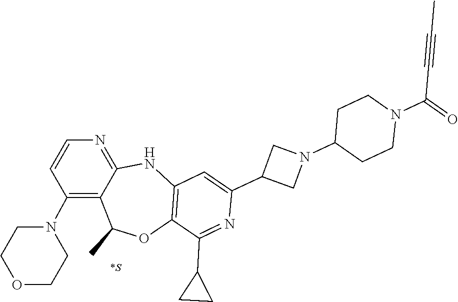 [SMILES: CC#CC(=O)N1CCC(N2CC(c3cc4c(c(C5CC5)n3)O[C@@H](C)c3c(N5CCOCC5)ccnc3N4)C2)CC1] |
| 375 | 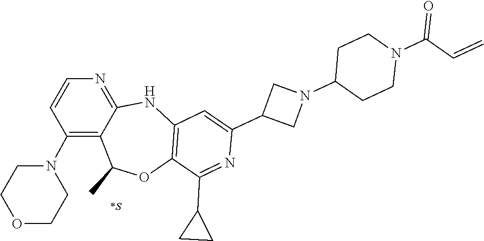 [SMILES: C=CC(=O)N1CCC(N2CC(c3cc4c(c(C5CC5)n3)O[C@@H](C)c3c(N5CCOCC5)ccnc3N4)C2)CC1] |
| 376 | 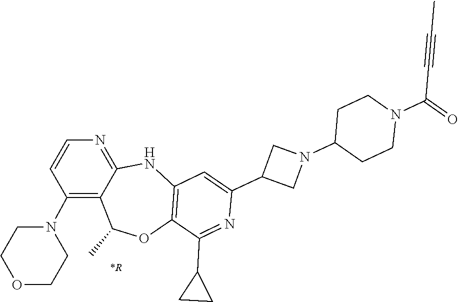 [SMILES: CC#CC(=O)N1CCC(N2CC(c3cc4c(c(C5CC5)n3)O[C@H](C)c3c(N5CCOCC5)ccnc3N4)C2)CC1] |
| 377 | 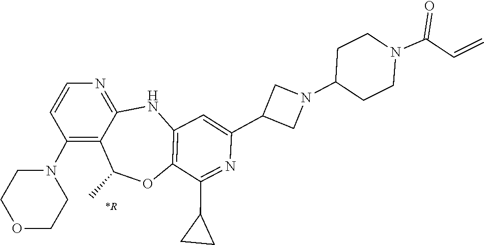 |
| 378 | 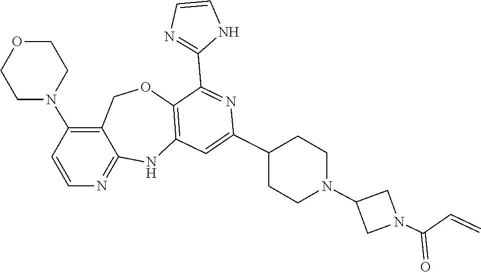 [SMILES: C=CC(=O)N1CC(N2CCC(c3cc4c(c(-c5ncc[nH]5)n3)OCc3c(N5CCOCC5)ccnc3N4)CC2)C1] |
| 379 | 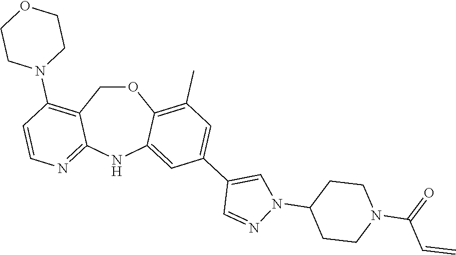 [SMILES: C=CC(=O)N1CCC(n2cc(-c3cc(C)c4c(c3)Nc3nccc(N5CCOCC5)c3CO4)cn2)CC1] |
| 380 | 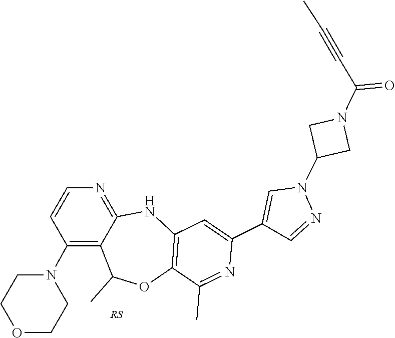 [SMILES: CC#CC(=O)N1CC(n2cc(-c3cc4c(c(C)n3)OC(C)c3c(N5CCOCC5)ccnc3N4)cn2)C1] |
| 381 | 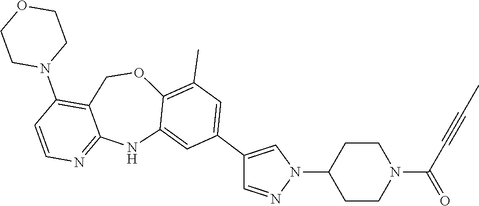 [SMILES: CC#CC(=O)N1CCC(n2cc(-c3cc(C)c4c(c3)Nc3nccc(N5CCOCC5)c3CO4)cn2)CC1] |
| 382 | 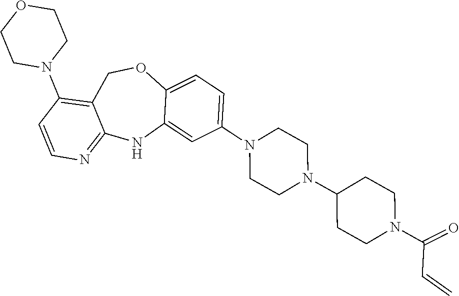 [SMILES: C=CC(=O)N1CCC(N2CCN(c3ccc4c(c3)Nc3nccc(N5CCOCC5)c3CO4)CC2)CC1] |
| 383 | 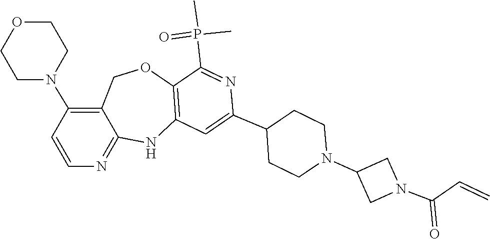 [SMILES: C=CC(=O)N1CC(N2CCC(c3cc4c(c(P(C)(C)=O)n3)OCc3c(N5CCOCC5)ccnc3N4)CC2)C1] |
| 384 | 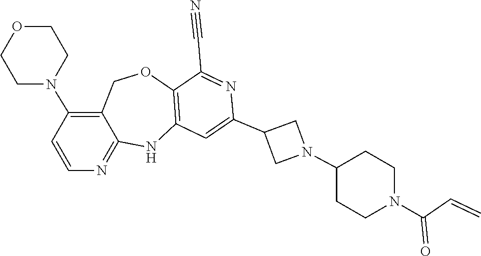 [SMILES: C=CC(=O)N1CCC(N2CC(c3cc4c(c(C#N)n3)OCc3c(N5CCOCC5)ccnc3N4)C2)CC1] |
| 385 | 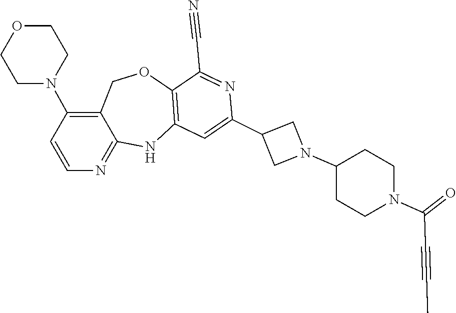 [SMILES: CC#CC(=O)N1CCC(N2CC(c3cc4c(c(C#N)n3)OCc3c(N5CCOCC5)ccnc3N4)C2)CC1] |
| 386 | 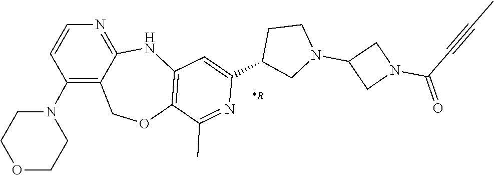 [SMILES: CC#CC(=O)N1CC(N2CC[C@@H](c3cc4c(c(C)n3)OCc3c(N5CCOCC5)ccnc3N4)C2)C1] |
| 387 | 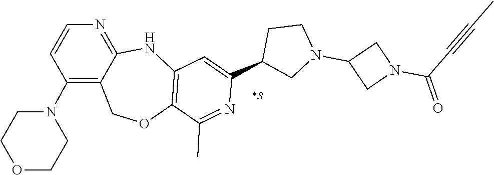 [SMILES: CC#CC(=O)N1CC(N2CC[C@H](c3cc4c(c(C)n3)OCc3c(N5CCOCC5)ccnc3N4)C2)C1] |
| 388 | 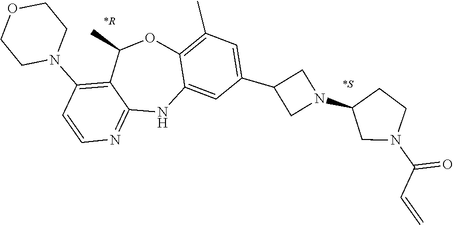 [SMILES: C=CC(=O)N1CC[C@H](N2CC(c3cc(C)c4c(c3)Nc3nccc(N5CCOCC5)c3[C@@H](C)O4)C2)C1] |
| 389 | 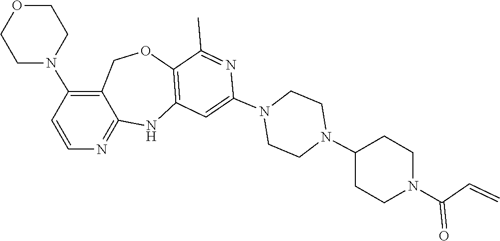 [SMILES: C=CC(=O)N1CCC(N2CCN(c3cc4c(c(C)n3)OCc3c(N5CCOCC5)ccnc3N4)CC2)CC1] |
| 390 | 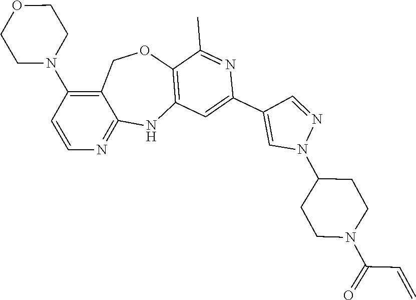 [SMILES: C=CC(=O)N1CCC(n2cc(-c3cc4c(c(C)n3)OCc3c(N5CCOCC5)ccnc3N4)cn2)CC1] |
| 391 | 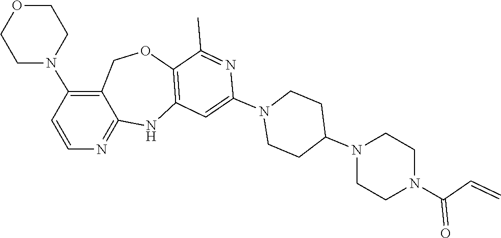 [SMILES: C=CC(=O)N1CCN(C2CCN(c3cc4c(c(C)n3)OCc3c(N5CCOCC5)ccnc3N4)CC2)CC1] |
| 392 | 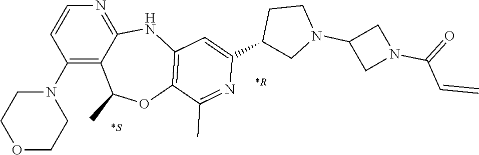 [SMILES: C=CC(=O)N1CC(N2CC[C@@H](c3cc4c(c(C)n3)O[C@@H](C)c3c(N5CCOCC5)ccnc3N4)C2)C1] |
| 393 | 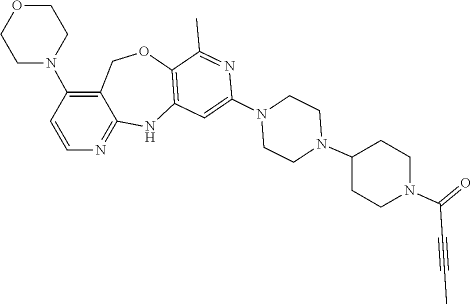 [SMILES: CC#CC(=O)N1CCC(N2CCN(c3cc4c(c(C)n3)OCc3c(N5CCOCC5)ccnc3N4)CC2)CC1] |
| 394 | 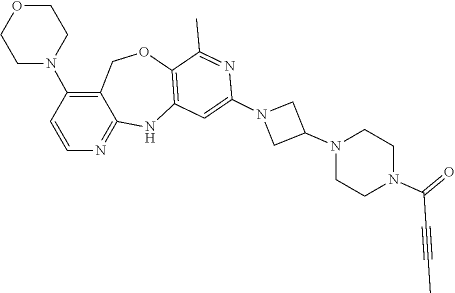 [SMILES: CC#CC(=O)N1CCN(C2CN(c3cc4c(c(C)n3)OCc3c(N5CCOCC5)ccnc3N4)C2)CC1] |
| 395 | 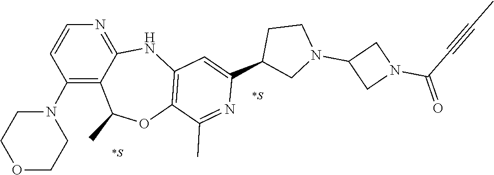 [SMILES: CC#CC(=O)N1CC(N2CC[C@H](c3cc4c(c(C)n3)O[C@@H](C)c3c(N5CCOCC5)ccnc3N4)C2)C1] |
| 396 | 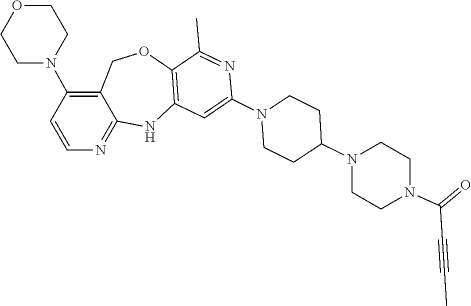 [SMILES: CC#CC(=O)N1CCN(C2CCN(c3cc4c(c(C)n3)OCc3c(N5CCOCC5)ccnc3N4)CC2)CC1] |
| 397 | 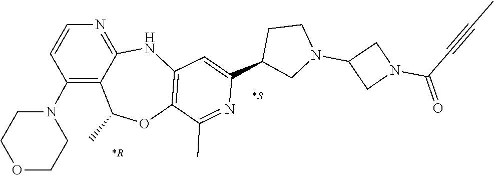 [SMILES: CC#CC(=O)N1CC(N2CC[C@H](c3cc4c(c(C)n3)O[C@H](C)c3c(N5CCOCC5)ccnc3N4)C2)C1] |
| 398 | 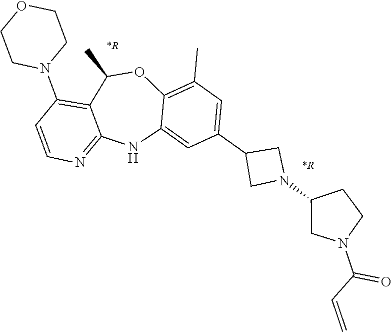 [SMILES: C=CC(=O)N1CC[C@@H](N2CC(c3cc(C)c4c(c3)Nc3nccc(N5CCOCC5)c3[C@@H](C)O4)C2)C1] |
| 399 | 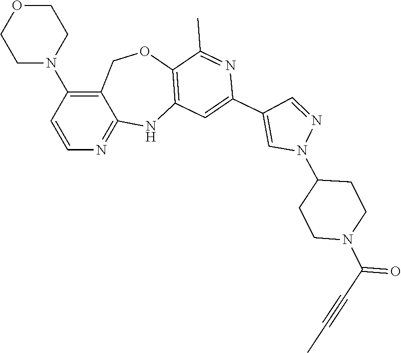 [SMILES: CC#CC(=O)N1CCC(n2cc(-c3cc4c(c(C)n3)OCc3c(N5CCOCC5)ccnc3N4)cn2)CC1] |
| 400 | 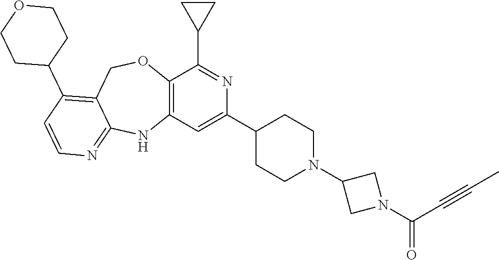 [SMILES: CC#CC(=O)N1CC(N2CCC(c3cc4c(c(C5CC5)n3)OCc3c(C5CCOCC5)ccnc3N4)CC2)C1] |
| 400 | 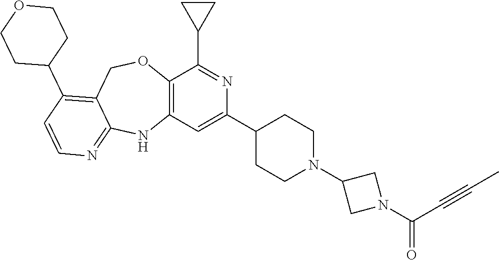 [SMILES: CC#CC(=O)N1CC(N2CCC(c3cc4c(c(C5CC5)n3)OCc3c(C5CCOCC5)ccnc3N4)CC2)C1] |
| 401 | 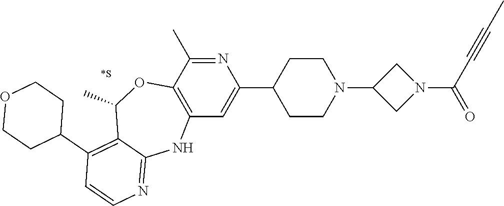 [SMILES: CC#CC(=O)N1CC(N2CCC(c3cc4c(c(C)n3)O[C@@H](C)c3c(C5CCOCC5)ccnc3N4)CC2)C1] |
| 402 | 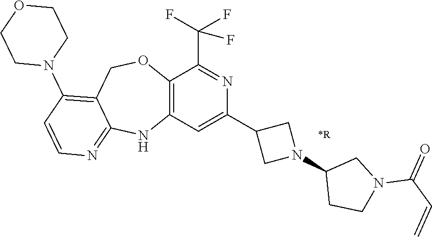 [SMILES: C=CC(=O)N1CC[C@@H](N2CC(c3cc4c(c(C(F)(F)F)n3)OCc3c(N5CCOCC5)ccnc3N4)C2)C1] |
| 403 | 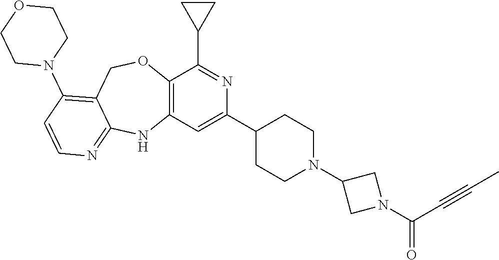 [SMILES: CC#CC(=O)N1CC(N2CCC(c3cc4c(c(C5CC5)n3)OCc3c(N5CCOCC5)ccnc3N4)CC2)C1] |
| 404 | 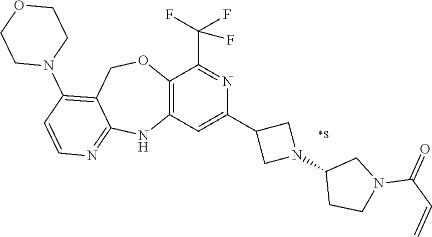 [SMILES: C=CC(=O)N1CC[C@H](N2CC(c3cc4c(c(C(F)(F)F)n3)OCc3c(N5CCOCC5)ccnc3N4)C2)C1] |
| 405 | 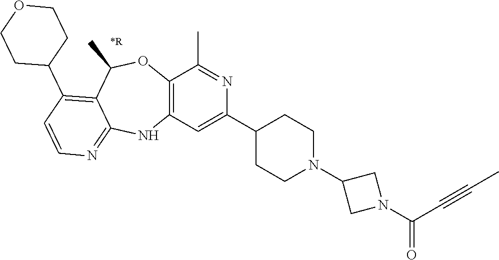 [SMILES: CC#CC(=O)N1CC(N2CCC(c3cc4c(c(C)n3)O[C@H](C)c3c(C5CCOCC5)ccnc3N4)CC2)C1] |
| 406 | 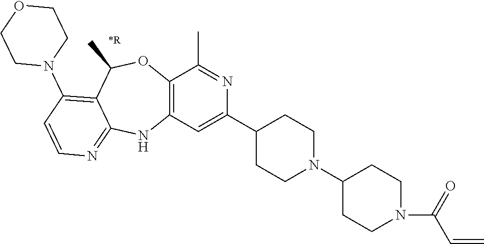 [SMILES: C=CC(=O)N1CCC(N2CCC(c3cc4c(c(C)n3)O[C@H](C)c3c(N5CCOCC5)ccnc3N4)CC2)CC1] |
| 407 | 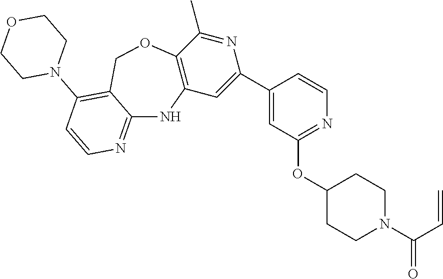 [SMILES: C=CC(=O)N1CCC(Oc2cc(-c3cc4c(c(C)n3)OCc3c(N5CCOCC5)ccnc3N4)ccn2)CC1] |
| 408 | 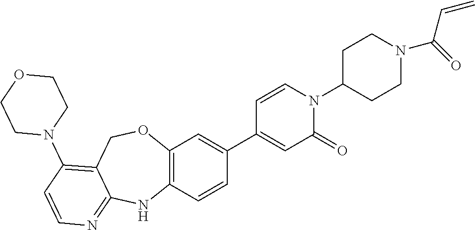 [SMILES: C=CC(=O)N1CCC(n2ccc(-c3ccc4c(c3)OCc3c(N5CCOCC5)ccnc3N4)cc2=O)CC1] |
| 409 | 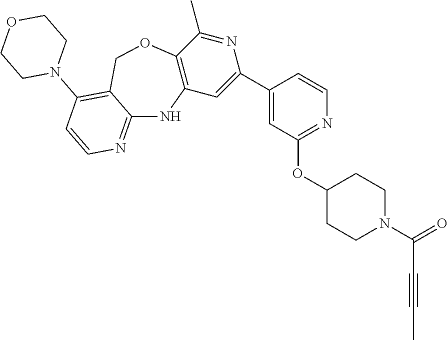 [SMILES: CC#CC(=O)N1CCC(Oc2cc(-c3cc4c(c(C)n3)OCc3c(N5CCOCC5)ccnc3N4)ccn2)CC1] |
| 410 | 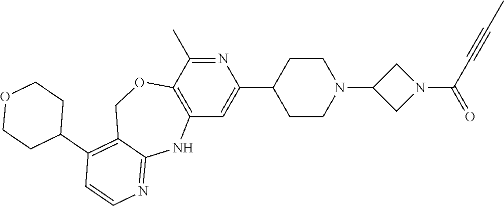 [SMILES: CC#CC(=O)N1CC(N2CCC(c3cc4c(c(C)n3)OCc3c(C5CCOCC5)ccnc3N4)CC2)C1] |
| 411 | 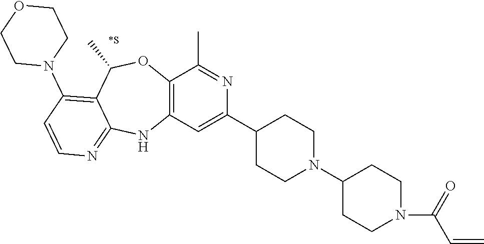 [SMILES: C=CC(=O)N1CCC(N2CCC(c3cc4c(c(C)n3)O[C@@H](C)c3c(N5CCOCC5)ccnc3N4)CC2)CC1] |
| 412 | 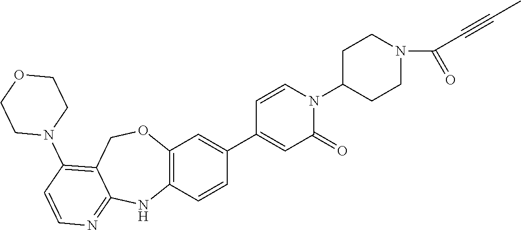 [SMILES: CC#CC(=O)N1CCC(n2ccc(-c3ccc4c(c3)OCc3c(N5CCOCC5)ccnc3N4)cc2=O)CC1] |
| 413 | 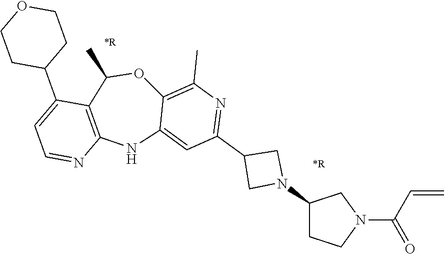 [SMILES: C=CC(=O)N1CC[C@@H](N2CC(c3cc4c(c(C)n3)O[C@H](C)c3c(C5CCOCC5)ccnc3N4)C2)C1] |
| 414 | 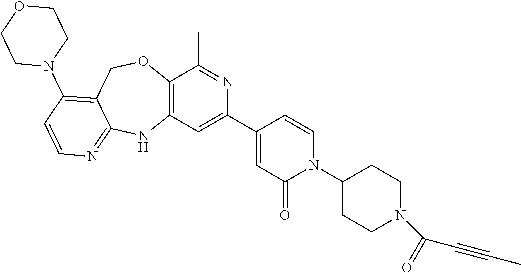 [SMILES: CC#CC(=O)N1CCC(n2ccc(-c3cc4c(c(C)n3)OCc3c(N5CCOCC5)ccnc3N4)cc2=O)CC1] |
| 415 | 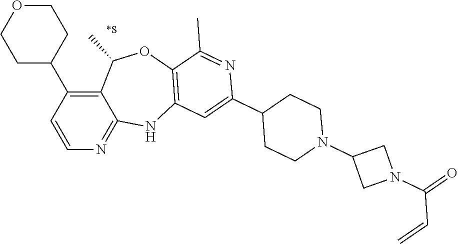 [SMILES: C=CC(=O)N1CC(N2CCC(c3cc4c(c(C)n3)O[C@@H](C)c3c(C5CCOCC5)ccnc3N4)CC2)C1] |
| 416 | 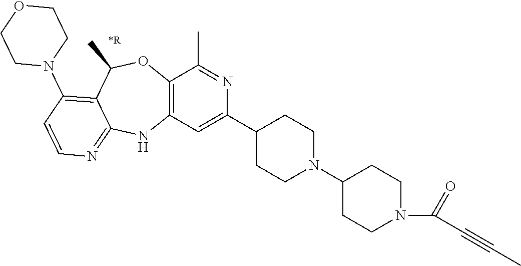 [SMILES: CC#CC(=O)N1CCC(N2CCC(c3cc4c(c(C)n3)O[C@H](C)c3c(N5CCOCC5)ccnc3N4)CC2)CC1] |
| 417 | 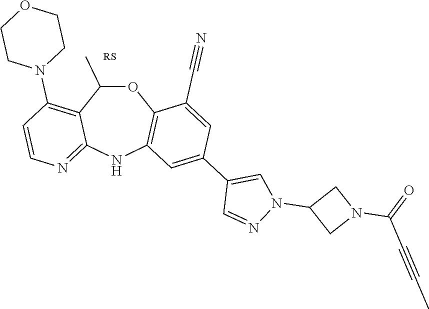 [SMILES: CC#CC(=O)N1CC(n2cc(-c3cc(C#N)c4c(c3)Nc3nccc(N5CCOCC5)c3C(C)O4)cn2)C1] |
| 418 | 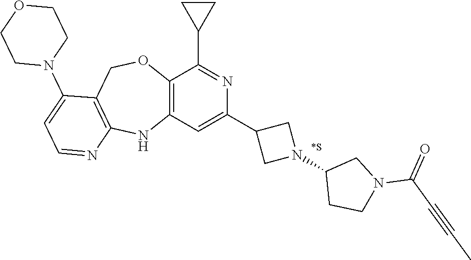 [SMILES: CC#CC(=O)N1CC[C@H](N2CC(c3cc4c(c(C5CC5)n3)OCc3c(N5CCOCC5)ccnc3N4)C2)C1] |
| 419 | 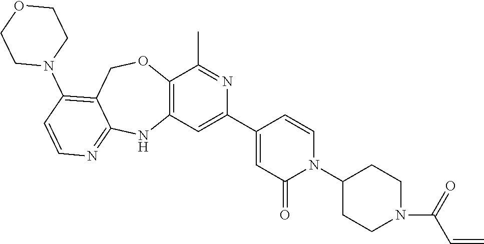 [SMILES: C=CC(=O)N1CCC(n2ccc(-c3cc4c(c(C)n3)OCc3c(N5CCOCC5)ccnc3N4)cc2=O)CC1] |
| 420 | 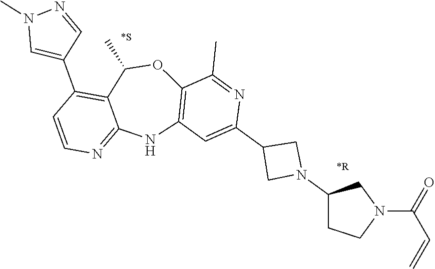 [SMILES: C=CC(=O)N1CC[C@@H](N2CC(c3cc4c(c(C)n3)O[C@@H](C)c3c(-c5cnn(C)c5)ccnc3N4)C2)C1] |
| 421 | 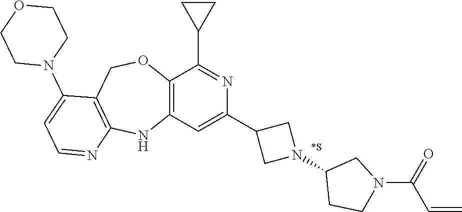 [SMILES: C=CC(=O)N1CC[C@H](N2CC(c3cc4c(c(C5CC5)n3)OCc3c(N5CCOCC5)ccnc3N4)C2)C1] |
| 422 | 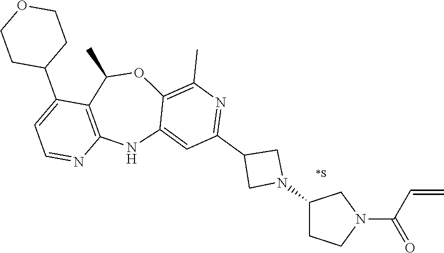 [SMILES: C=CC(=O)N1CC[C@H](N2CC(c3cc4c(c(C)n3)O[C@H](C)c3c(C5CCOCC5)ccnc3N4)C2)C1] |
| 423 | 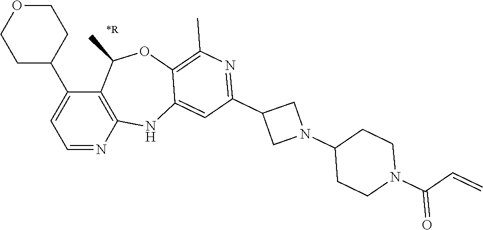 [SMILES: C=CC(=O)N1CCC(N2CC(c3cc4c(c(C)n3)O[C@H](C)c3c(C5CCOCC5)ccnc3N4)C2)CC1] |
| 424 | 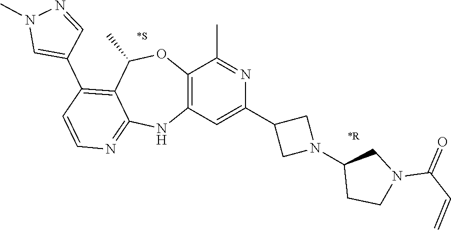 [SMILES: C=CC(=O)N1CC[C@@H](N2CC(c3cc4c(c(C)n3)O[C@@H](C)c3c(-c5cnn(C)c5)ccnc3N4)C2)C1] |
| 425 | 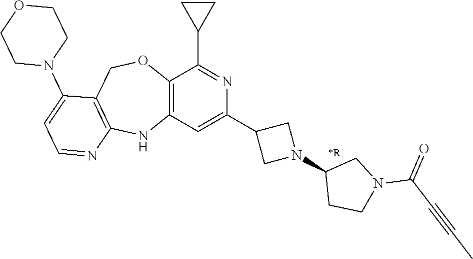 [SMILES: CC#CC(=O)N1CC[C@@H](N2CC(c3cc4c(c(C5CC5)n3)OCc3c(N5CCOCC5)ccnc3N4)C2)C1] |
| 426 | 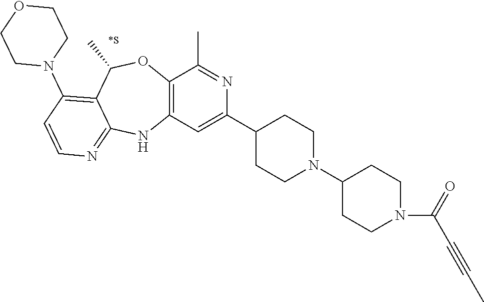 [SMILES: CC#CC(=O)N1CCC(N2CCC(c3cc4c(c(C)n3)O[C@@H](C)c3c(N5CCOCC5)ccnc3N4)CC2)CC1] |
| 427 | 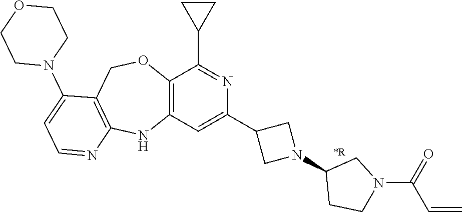 [SMILES: C=CC(=O)N1CC[C@@H](N2CC(c3cc4c(c(C5CC5)n3)OCc3c(N5CCOCC5)ccnc3N4)C2)C1] |
| 428 | 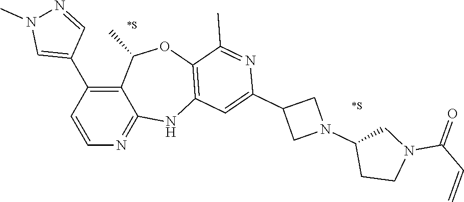 [SMILES: C=CC(=O)N1CC[C@H](N2CC(c3cc4c(c(C)n3)O[C@@H](C)c3c(-c5cnn(C)c5)ccnc3N4)C2)C1] |
| 429 | 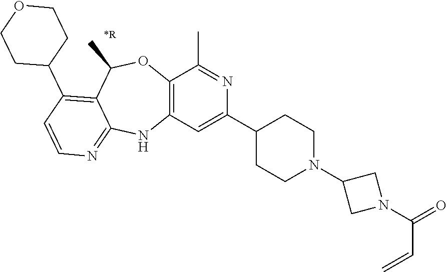 [SMILES: C=CC(=O)N1CC(N2CCC(c3cc4c(c(C)n3)O[C@H](C)c3c(C5CCOCC5)ccnc3N4)CC2)C1] |
| 430 | 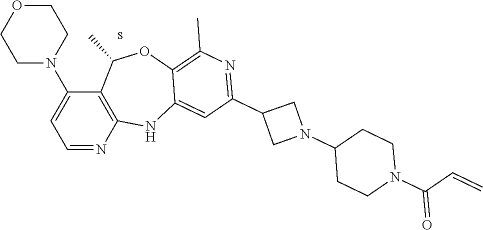 |
| 431 | 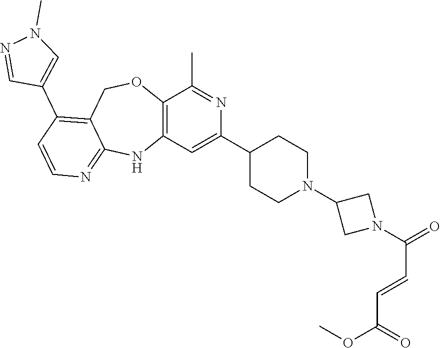 [SMILES: COC(=O)/C=C/C(=O)N1CC(N2CCC(c3cc4c(c(C)n3)OCc3c(-c5cnn(C)c5)ccnc3N4)CC2)C1] |
| 432 | 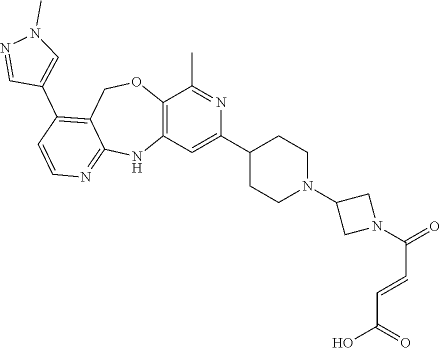 [SMILES: Cc1nc(C2CCN(C3CN(C(=O)/C=C/C(=O)O)C3)CC2)cc2c1OCc1c(-c3cnn(C)c3)ccnc1N2] |
| 433 | 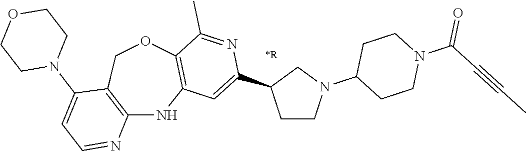 [SMILES: CC#CC(=O)N1CCC(N2CC[C@@H](c3cc4c(c(C)n3)OCc3c(N5CCOCC5)ccnc3N4)C2)CC1] |
| 434 | 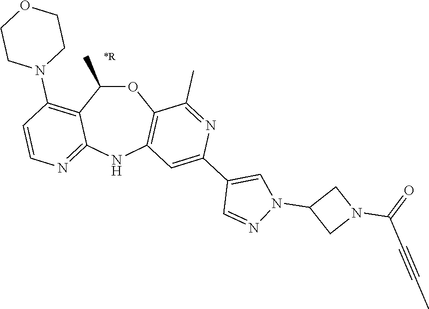 [SMILES: CC#CC(=O)N1CC(n2cc(-c3cc4c(c(C)n3)O[C@H](C)c3c(N5CCOCC5)ccnc3N4)cn2)C1] |
| 435 | 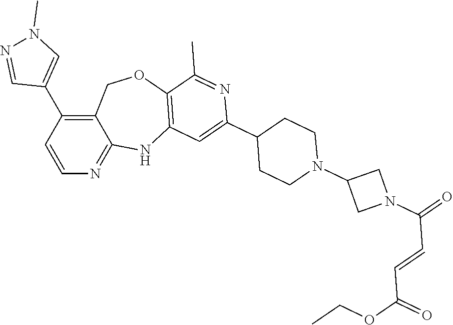 [SMILES: CCOC(=O)/C=C/C(=O)N1CC(N2CCC(c3cc4c(c(C)n3)OCc3c(-c5cnn(C)c5)ccnc3N4)CC2)C1] |
| 436 | 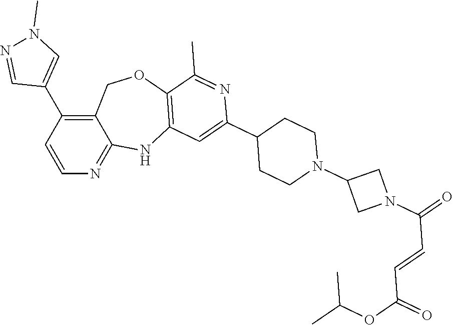 [SMILES: Cc1nc(C2CCN(C3CN(C(=O)/C=C/C(=O)OC(C)C)C3)CC2)cc2c1OCc1c(-c3cnn(C)c3)ccnc1N2] |
| 437 | 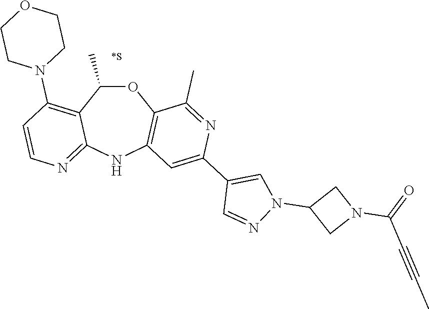 [SMILES: CC#CC(=O)N1CC(n2cc(-c3cc4c(c(C)n3)O[C@@H](C)c3c(N5CCOCC5)ccnc3N4)cn2)C1] |
| 438 | 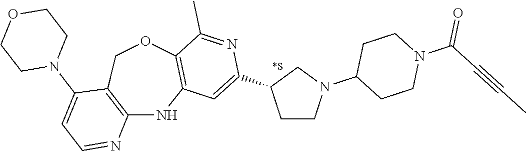 [SMILES: CC#CC(=O)N1CCC(N2CC[C@H](c3cc4c(c(C)n3)OCc3c(N5CCOCC5)ccnc3N4)C2)CC1] |
| 439 | 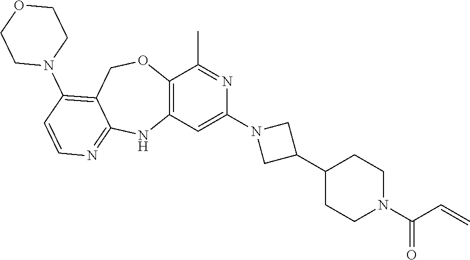 [SMILES: C=CC(=O)N1CCC(C2CN(c3cc4c(c(C)n3)OCc3c(N5CCOCC5)ccnc3N4)C2)CC1] |
| 440 | 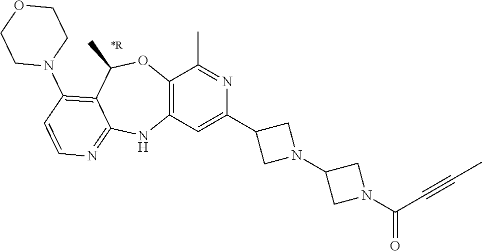 [SMILES: CC#CC(=O)N1CC(N2CC(c3cc4c(c(C)n3)O[C@H](C)c3c(N5CCOCC5)ccnc3N4)C2)C1] |
| 441 | 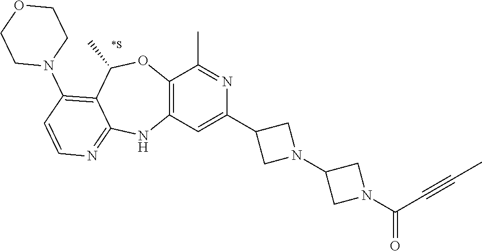 [SMILES: CC#CC(=O)N1CC(N2CC(c3cc4c(c(C)n3)O[C@@H](C)c3c(N5CCOCC5)ccnc3N4)C2)C1] |
| 442 | 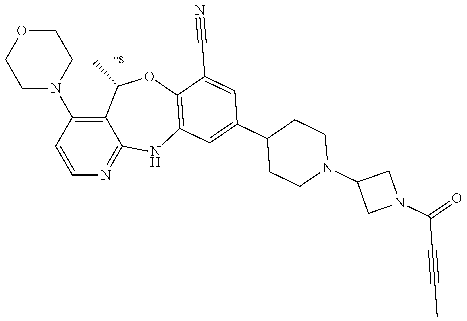 [SMILES: CC#CC(=O)N1CC(N2CCC(c3cc(C#N)c4c(c3)Nc3nccc(N5CCOCC5)c3[C@H](C)O4)CC2)C1] |
| 443 | 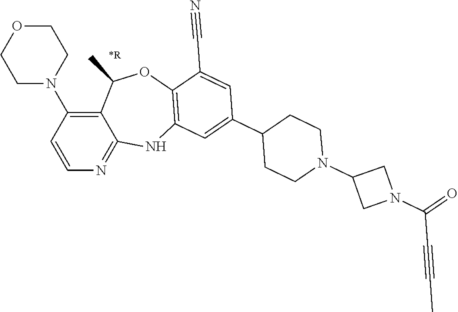 [SMILES: CC#CC(=O)N1CC(N2CCC(c3cc(C#N)c4c(c3)Nc3nccc(N5CCOCC5)c3[C@@H](C)O4)CC2)C1] |
| 444 | 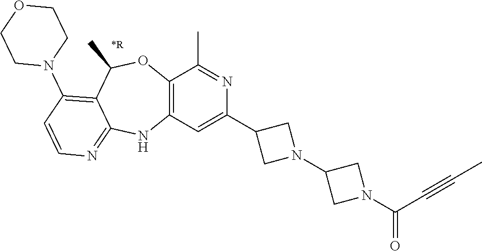 [SMILES: CC#CC(=O)N1CC(N2CC(c3cc4c(c(C)n3)O[C@H](C)c3c(N5CCOCC5)ccnc3N4)C2)C1] |
| 445 | 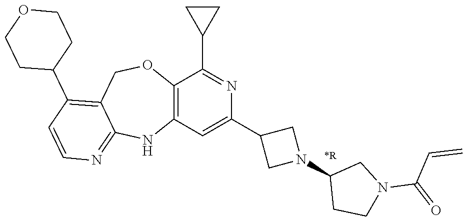 [SMILES: C=CC(=O)N1CC[C@@H](N2CC(c3cc4c(c(C5CC5)n3)OCc3c(C5CCOCC5)ccnc3N4)C2)C1] |
| 446 | 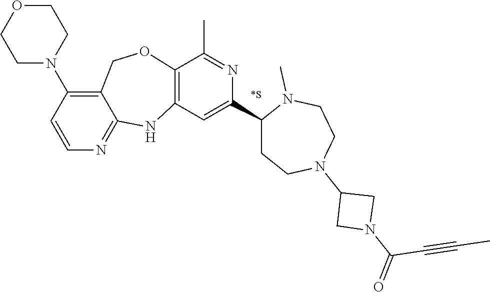 [SMILES: CC#CC(=O)N1CC(N2CC[C@@H](c3cc4c(c(C)n3)OCc3c(N5CCOCC5)ccnc3N4)N(C)CC2)C1] |
| 447 | 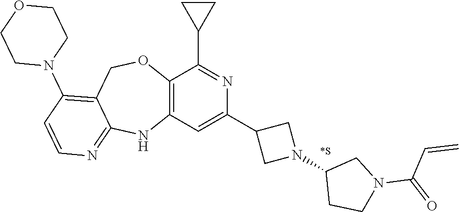 [SMILES: C=CC(=O)N1CC[C@H](N2CC(c3cc4c(c(C5CC5)n3)OCc3c(N5CCOCC5)ccnc3N4)C2)C1] |
| 448 | 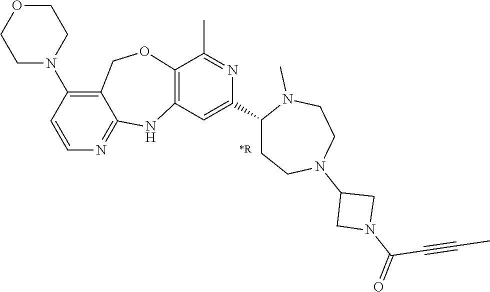 [SMILES: CC#CC(=O)N1CC(N2CC[C@H](c3cc4c(c(C)n3)OCc3c(N5CCOCC5)ccnc3N4)N(C)CC2)C1] |
| 449 | 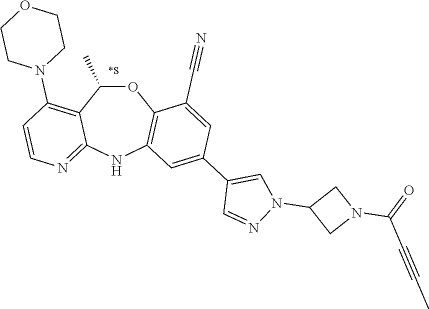 [SMILES: CC#CC(=O)N1CC(n2cc(-c3cc(C#N)c4c(c3)Nc3nccc(N5CCOCC5)c3[C@H](C)O4)cn2)C1] |
| 450 | 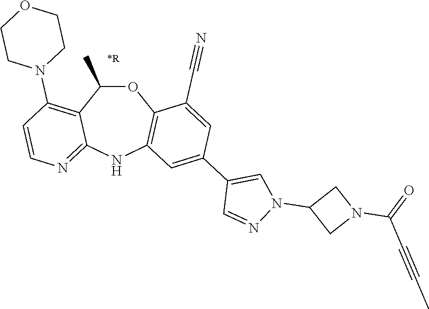 [SMILES: CC#CC(=O)N1CC(n2cc(-c3cc(C#N)c4c(c3)Nc3nccc(N5CCOCC5)c3[C@@H](C)O4)cn2)C1] |
| 451 | 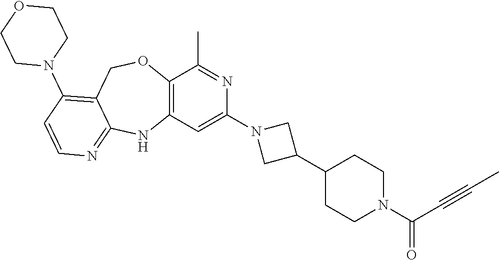 [SMILES: CC#CC(=O)N1CCC(C2CN(c3cc4c(c(C)n3)OCc3c(N5CCOCC5)ccnc3N4)C2)CC1] |
| 452 | 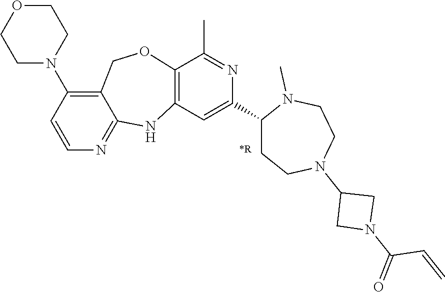 [SMILES: C=CC(=O)N1CC(N2CC[C@H](c3cc4c(c(C)n3)OCc3c(N5CCOCC5)ccnc3N4)N(C)CC2)C1] |
| 453 | 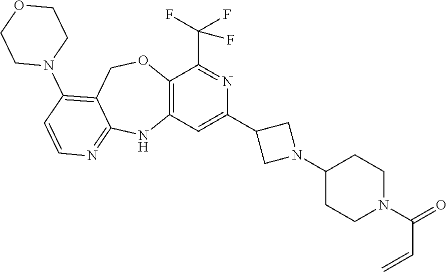 [SMILES: C=CC(=O)N1CCC(N2CC(c3cc4c(c(C(F)(F)F)n3)OCc3c(N5CCOCC5)ccnc3N4)C2)CC1] |
| 454 | 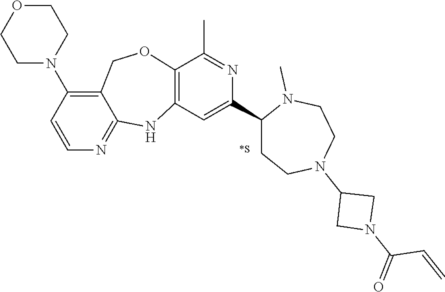 [SMILES: C=CC(=O)N1CC(N2CC[C@@H](c3cc4c(c(C)n3)OCc3c(N5CCOCC5)ccnc3N4)N(C)CC2)C1] |
| 455 | 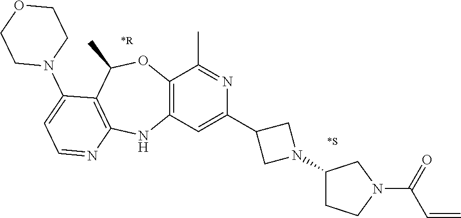 [SMILES: C=CC(=O)N1CC[C@H](N2CC(c3cc4c(c(C)n3)O[C@H](C)c3c(N5CCOCC5)ccnc3N4)C2)C1] |
| 456 | 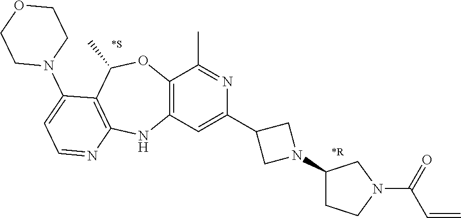 [SMILES: C=CC(=O)N1CC[C@@H](N2CC(c3cc4c(c(C)n3)O[C@@H](C)c3c(N5CCOCC5)ccnc3N4)C2)C1] |
| 457 | 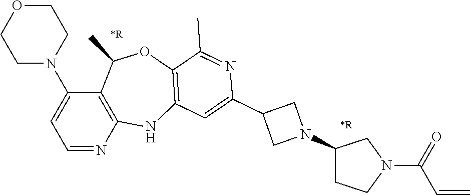 [SMILES: C=CC(=O)N1CC[C@@H](N2CC(c3cc4c(c(C)n3)O[C@H](C)c3c(N5CCOCC5)ccnc3N4)C2)C1] |
| 458 | 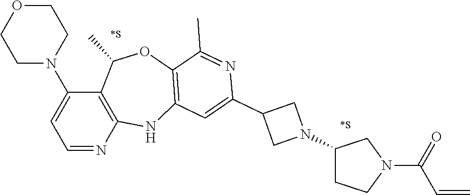 [SMILES: C=CC(=O)N1CC[C@H](N2CC(c3cc4c(c(C)n3)O[C@@H](C)c3c(N5CCOCC5)ccnc3N4)C2)C1] |
| 459 | 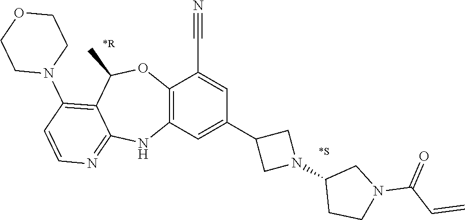 [SMILES: C=CC(=O)N1CC[C@H](N2CC(c3cc(C#N)c4c(c3)Nc3nccc(N5CCOCC5)c3[C@@H](C)O4)C2)C1] |
| 460 | 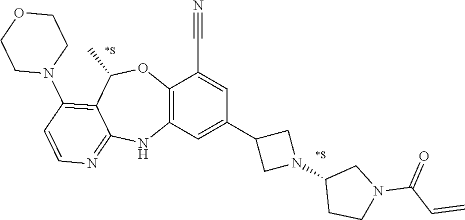 [SMILES: C=CC(=O)N1CC[C@H](N2CC(c3cc(C#N)c4c(c3)Nc3nccc(N5CCOCC5)c3[C@H](C)O4)C2)C1] |
| 461 | 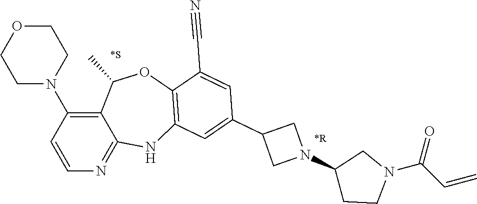 [SMILES: C=CC(=O)N1CC[C@@H](N2CC(c3cc(C#N)c4c(c3)Nc3nccc(N5CCOCC5)c3[C@H](C)O4)C2)C1] |
| 462 | 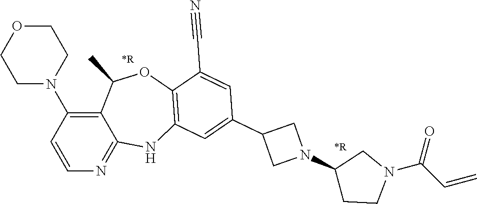 [SMILES: C=CC(=O)N1CC[C@@H](N2CC(c3cc(C#N)c4c(c3)Nc3nccc(N5CCOCC5)c3[C@@H](C)O4)C2)C1] |
| 463 | 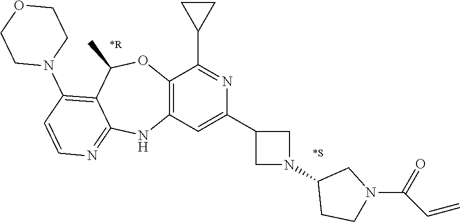 [SMILES: C=CC(=O)N1CC[C@H](N2CC(c3cc4c(c(C5CC5)n3)O[C@H](C)c3c(N5CCOCC5)ccnc3N4)C2)C1] |
| 464 | 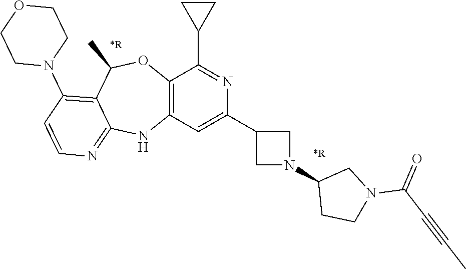 [SMILES: CC#CC(=O)N1CC[C@@H](N2CC(c3cc4c(c(C5CC5)n3)O[C@H](C)c3c(N5CCOCC5)ccnc3N4)C2)C1] |
| 465 | 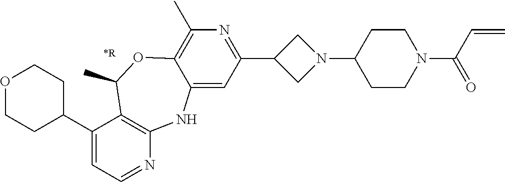 [SMILES: C=CC(=O)N1CCC(N2CC(c3cc4c(c(C)n3)O[C@H](C)c3c(C5CCOCC5)ccnc3N4)C2)CC1] |
| 466 | 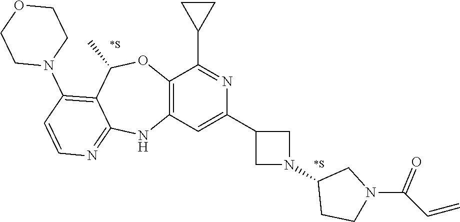 [SMILES: C=CC(=O)N1CC[C@H](N2CC(c3cc4c(c(C5CC5)n3)O[C@@H](C)c3c(N5CCOCC5)ccnc3N4)C2)C1] |
| 467 | 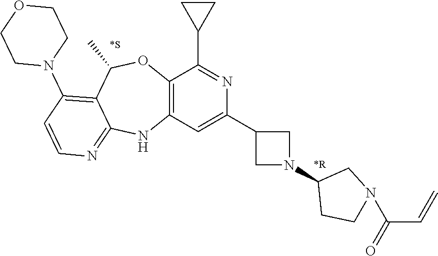 [SMILES: C=CC(=O)N1CC[C@@H](N2CC(c3cc4c(c(C5CC5)n3)O[C@@H](C)c3c(N5CCOCC5)ccnc3N4)C2)C1] |
| 468 | 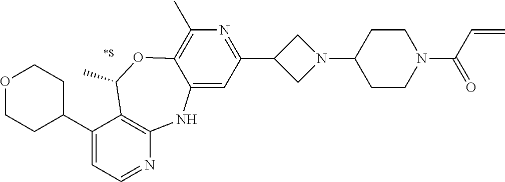 [SMILES: C=CC(=O)N1CCC(N2CC(c3cc4c(c(C)n3)O[C@@H](C)c3c(C5CCOCC5)ccnc3N4)C2)CC1] |
| 469 | 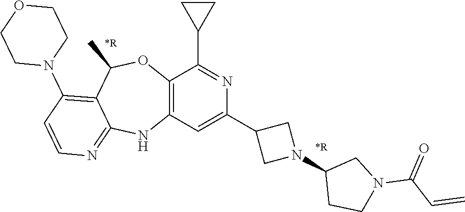 [SMILES: C=CC(=O)N1CC[C@@H](N2CC(c3cc4c(c(C5CC5)n3)O[C@H](C)c3c(N5CCOCC5)ccnc3N4)C2)C1] |
| 470 | 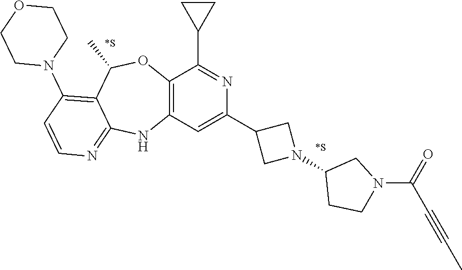 [SMILES: CC#CC(=O)N1CC[C@H](N2CC(c3cc4c(c(C5CC5)n3)O[C@@H](C)c3c(N5CCOCC5)ccnc3N4)C2)C1] |
| 471 | 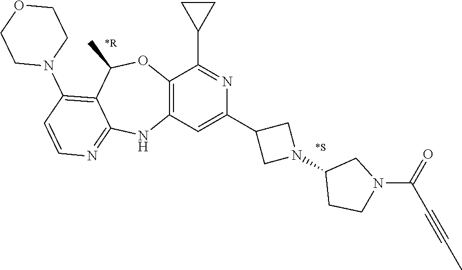 [SMILES: CC#CC(=O)N1CC[C@H](N2CC(c3cc4c(c(C5CC5)n3)O[C@H](C)c3c(N5CCOCC5)ccnc3N4)C2)C1] |
| 472 | 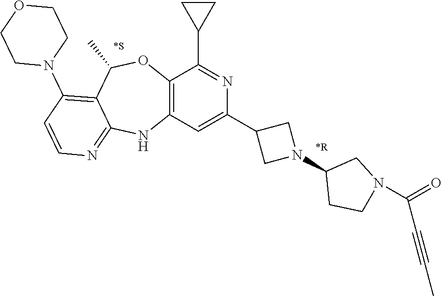 [SMILES: CC#CC(=O)N1CC[C@@H](N2CC(c3cc4c(c(C5CC5)n3)O[C@@H](C)c3c(N5CCOCC5)ccnc3N4)C2)C1] |
| 473 | 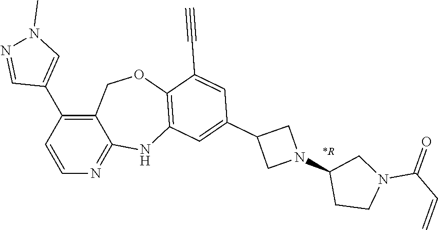 [SMILES: C#Cc1cc(C2CN([C@@H]3CCN(C(=O)C=C)C3)C2)cc2c1OCc1c(-c3cnn(C)c3)ccnc1N2] |
| 474 | 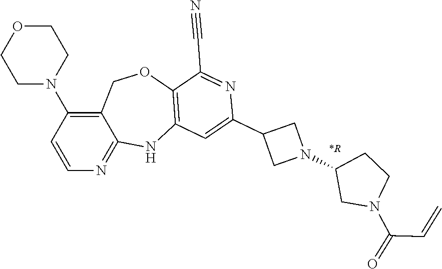 [SMILES: C=CC(=O)N1CC[C@@H](N2CC(c3cc4c(c(C#N)n3)OCc3c(N5CCOCC5)ccnc3N4)C2)C1] |
| 475 | 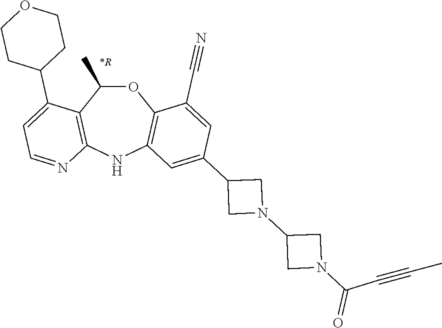 [SMILES: CC#CC(=O)N1CC(N2CC(c3cc(C#N)c4c(c3)Nc3nccc(C5CCOCC5)c3[C@@H](C)O4)C2)C1] |
| 476 | 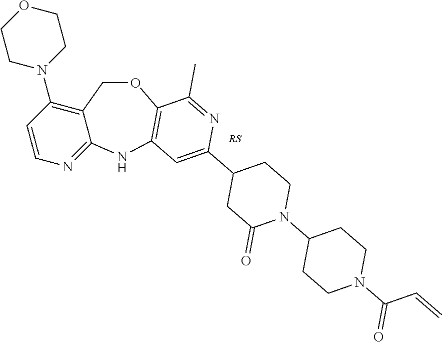 [SMILES: C=CC(=O)N1CCC(N2CCC(c3cc4c(c(C)n3)OCc3c(N5CCOCC5)ccnc3N4)CC2=O)CC1] |
| 477 | 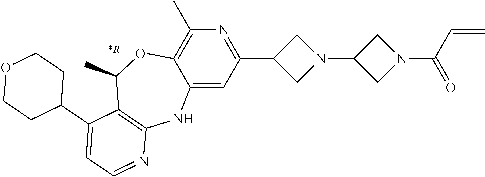 [SMILES: C=CC(=O)N1CC(N2CC(c3cc4c(c(C)n3)O[C@H](C)c3c(C5CCOCC5)ccnc3N4)C2)C1] |
| 478 | 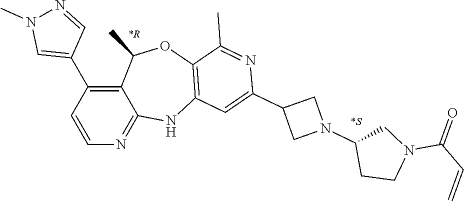 [SMILES: C=CC(=O)N1CC[C@H](N2CC(c3cc4c(c(C)n3)O[C@H](C)c3c(-c5cnn(C)c5)ccnc3N4)C2)C1] |
| 479 | 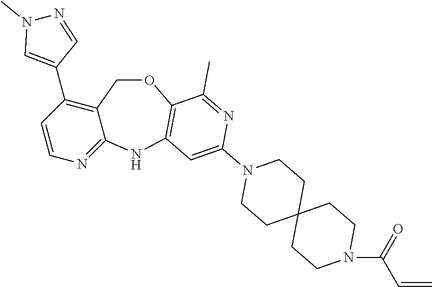 [SMILES: C=CC(=O)N1CCC2(CC1)CCN(c1cc3c(c(C)n1)OCc1c(-c4cnn(C)c4)ccnc1N3)CC2] |
| 480 | 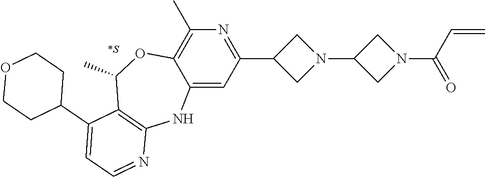 [SMILES: C=CC(=O)N1CC(N2CC(c3cc4c(c(C)n3)O[C@@H](C)c3c(C5CCOCC5)ccnc3N4)C2)C1] |
| 481 | 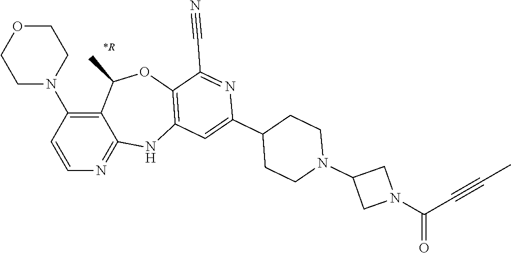 [SMILES: CC#CC(=O)N1CC(N2CCC(c3cc4c(c(C#N)n3)O[C@H](C)c3c(N5CCOCC5)ccnc3N4)CC2)C1] |
| 482 | 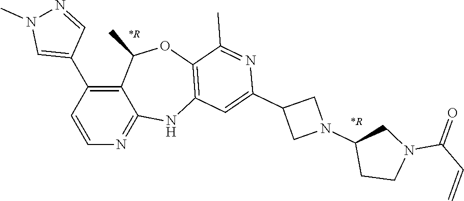 [SMILES: C=CC(=O)N1CC[C@@H](N2CC(c3cc4c(c(C)n3)O[C@H](C)c3c(-c5cnn(C)c5)ccnc3N4)C2)C1] |
| 483 | 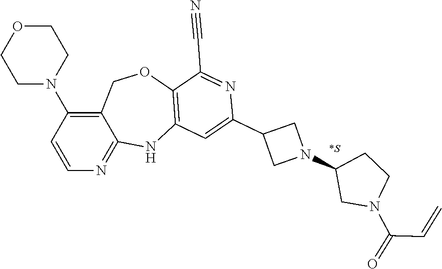 [SMILES: C=CC(=O)N1CC[C@H](N2CC(c3cc4c(c(C#N)n3)OCc3c(N5CCOCC5)ccnc3N4)C2)C1] |
| 484 | 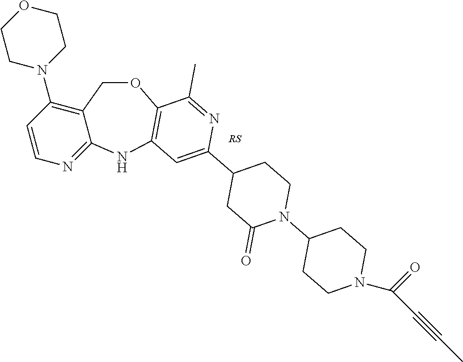 [SMILES: CC#CC(=O)N1CCC(N2CCC(c3cc4c(c(C)n3)OCc3c(N5CCOCC5)ccnc3N4)CC2=O)CC1] |
| 485 | 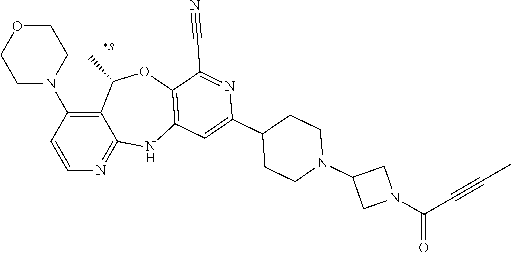 [SMILES: CC#CC(=O)N1CC(N2CCC(c3cc4c(c(C#N)n3)O[C@@H](C)c3c(N5CCOCC5)ccnc3N4)CC2)C1] |
| 486 | 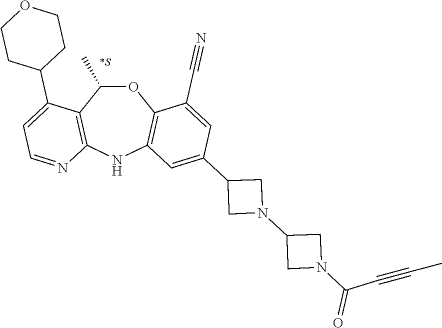 [SMILES: CC#CC(=O)N1CC(N2CC(c3cc(C#N)c4c(c3)Nc3nccc(C5CCOCC5)c3[C@H](C)O4)C2)C1] |
| 487 | 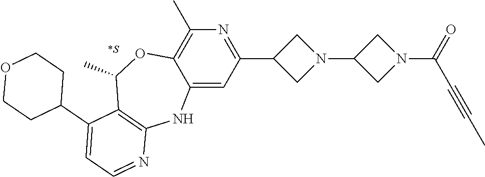 [SMILES: CC#CC(=O)N1CC(N2CC(c3cc4c(c(C)n3)O[C@@H](C)c3c(C5CCOCC5)ccnc3N4)C2)C1] |
| 488 | 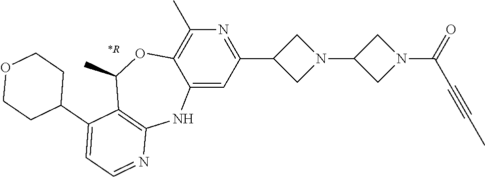 [SMILES: CC#CC(=O)N1CC(N2CC(c3cc4c(c(C)n3)O[C@H](C)c3c(C5CCOCC5)ccnc3N4)C2)C1] |
| 489 | 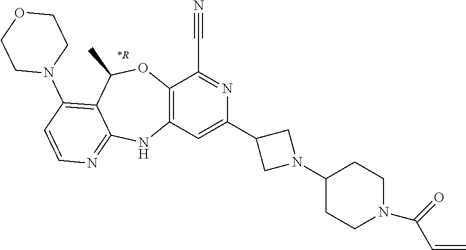 [SMILES: C=CC(=O)N1CCC(N2CC(c3cc4c(c(C#N)n3)O[C@H](C)c3c(N5CCOCC5)ccnc3N4)C2)CC1] |
| 490 | 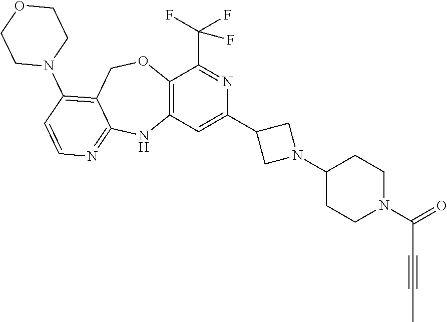 [SMILES: CC#CC(=O)N1CCC(N2CC(c3cc4c(c(C(F)(F)F)n3)OCc3c(N5CCOCC5)ccnc3N4)C2)CC1] |
| 491 | 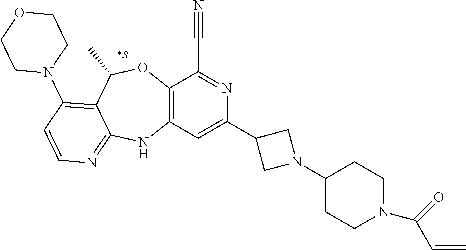 [SMILES: C=CC(=O)N1CCC(N2CC(c3cc4c(c(C#N)n3)O[C@@H](C)c3c(N5CCOCC5)ccnc3N4)C2)CC1] |
| 492 | 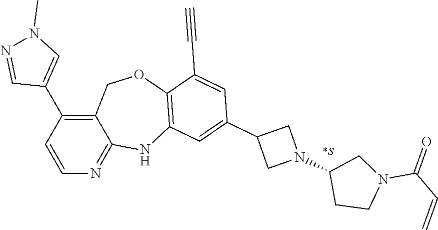 [SMILES: C#Cc1cc(C2CN([C@H]3CCN(C(=O)C=C)C3)C2)cc2c1OCc1c(-c3cnn(C)c3)ccnc1N2] |
| 493 | 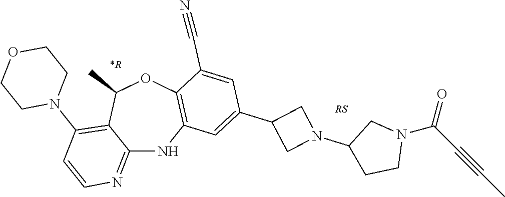 [SMILES: CC#CC(=O)N1CCC(N2CC(c3cc(C#N)c4c(c3)Nc3nccc(N5CCOCC5)c3[C@@H](C)O4)C2)C1] |
| 494 | 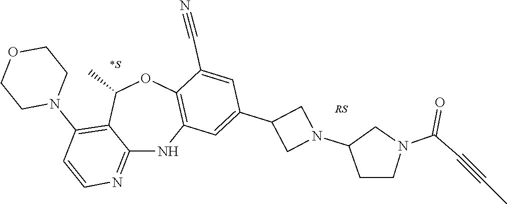 [SMILES: CC#CC(=O)N1CCC(N2CC(c3cc(C#N)c4c(c3)Nc3nccc(N5CCOCC5)c3[C@H](C)O4)C2)C1] |
| 495 | 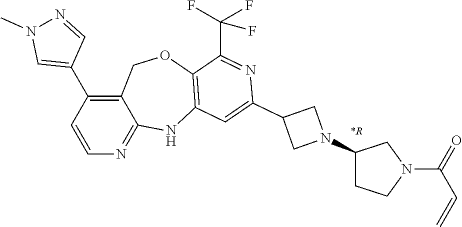 [SMILES: C=CC(=O)N1CC[C@@H](N2CC(c3cc4c(c(C(F)(F)F)n3)OCc3c(-c5cnn(C)c5)ccnc3N4)C2)C1] |
| 496 | 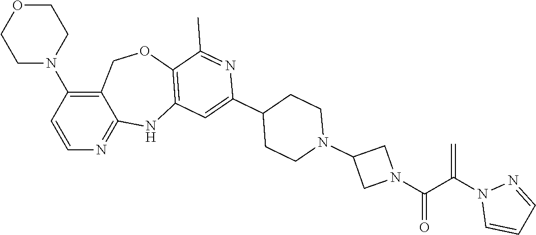 [SMILES: C=C(C(=O)N1CC(N2CCC(c3cc4c(c(C)n3)OCc3c(N5CCOCC5)ccnc3N4)CC2)C1)n1cccn1] |
| 497 | 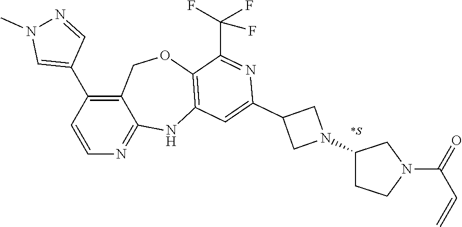 [SMILES: C=CC(=O)N1CC[C@H](N2CC(c3cc4c(c(C(F)(F)F)n3)OCc3c(-c5cnn(C)c5)ccnc3N4)C2)C1] |
| 498 | 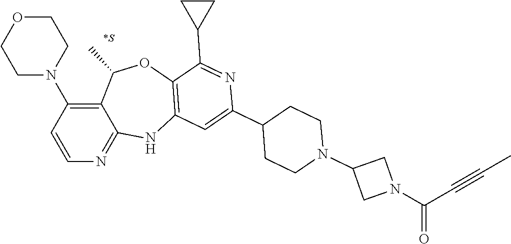 [SMILES: CC#CC(=O)N1CC(N2CCC(c3cc4c(c(C5CC5)n3)O[C@@H](C)c3c(N5CCOCC5)ccnc3N4)CC2)C1] |
| 499 | 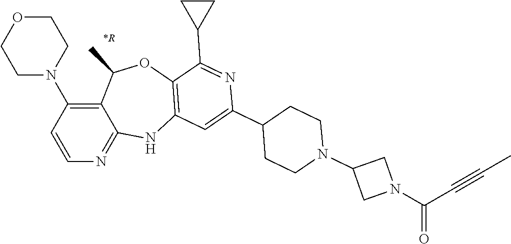 [SMILES: CC#CC(=O)N1CC(N2CCC(c3cc4c(c(C5CC5)n3)O[C@H](C)c3c(N5CCOCC5)ccnc3N4)CC2)C1] |
| 500 | 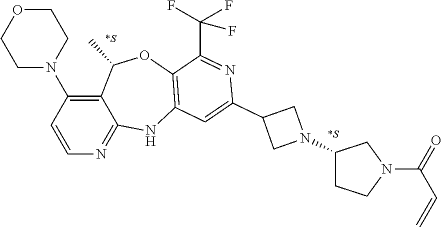 [SMILES: C=CC(=O)N1CC[C@H](N2CC(c3cc4c(c(C(F)(F)F)n3)O[C@@H](C)c3c(N5CCOCC5)ccnc3N4)C2)C1] |
| 501 | 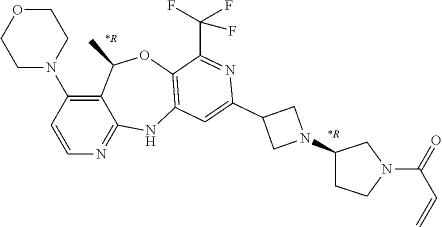 [SMILES: C=CC(=O)N1CC[C@@H](N2CC(c3cc4c(c(C(F)(F)F)n3)O[C@H](C)c3c(N5CCOCC5)ccnc3N4)C2)C1] |
| 502 | 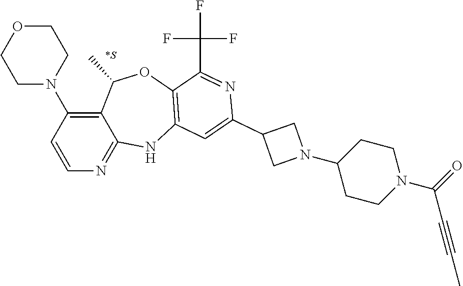 [SMILES: CC#CC(=O)N1CCC(N2CC(c3cc4c(c(C(F)(F)F)n3)O[C@@H](C)c3c(N5CCOCC5)ccnc3N4)C2)CC1] |
| 503 | 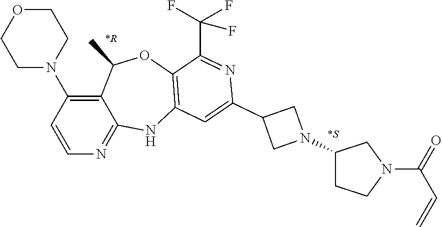 [SMILES: C=CC(=O)N1CC[C@H](N2CC(c3cc4c(c(C(F)(F)F)n3)O[C@H](C)c3c(N5CCOCC5)ccnc3N4)C2)C1] |
| 504 | 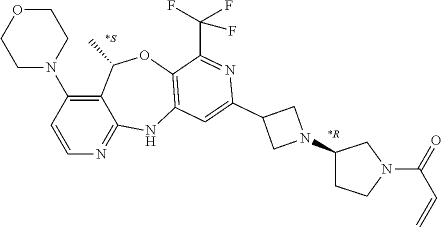 [SMILES: C=CC(=O)N1CC[C@@H](N2CC(c3cc4c(c(C(F)(F)F)n3)O[C@@H](C)c3c(N5CCOCC5)ccnc3N4)C2)C1] |
| 505 | 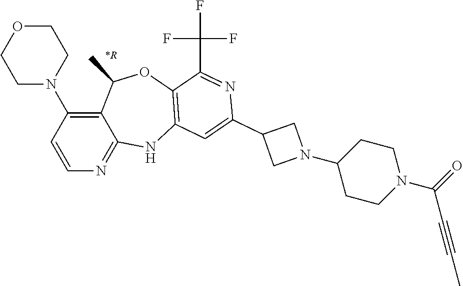 [SMILES: CC#CC(=O)N1CCC(N2CC(c3cc4c(c(C(F)(F)F)n3)O[C@H](C)c3c(N5CCOCC5)ccnc3N4)C2)CC1] |
| 506 | 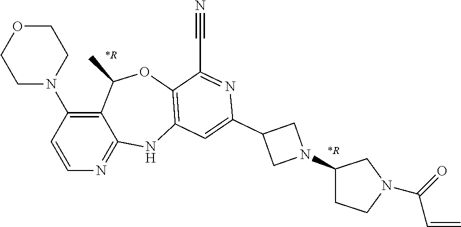 [SMILES: C=CC(=O)N1CC[C@@H](N2CC(c3cc4c(c(C#N)n3)O[C@H](C)c3c(N5CCOCC5)ccnc3N4)C2)C1] |
| 507 | 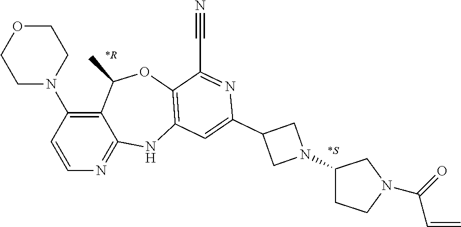 [SMILES: C=CC(=O)N1CC[C@H](N2CC(c3cc4c(c(C#N)n3)O[C@H](C)c3c(N5CCOCC5)ccnc3N4)C2)C1] |
| 508 | 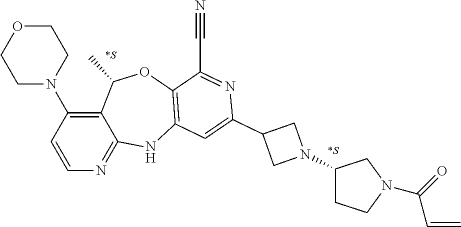 [SMILES: C=CC(=O)N1CC[C@H](N2CC(c3cc4c(c(C#N)n3)O[C@@H](C)c3c(N5CCOCC5)ccnc3N4)C2)C1] |
| 509 | 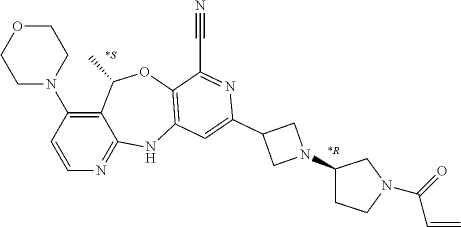 [SMILES: C=CC(=O)N1CC[C@@H](N2CC(c3cc4c(c(C#N)n3)O[C@@H](C)c3c(N5CCOCC5)ccnc3N4)C2)C1] |
| 510 | 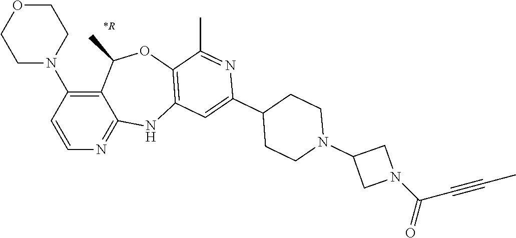 [SMILES: CC#CC(=O)N1CC(N2CCC(c3cc4c(c(C)n3)O[C@H](C)c3c(N5CCOCC5)ccnc3N4)CC2)C1] |
| 511 | 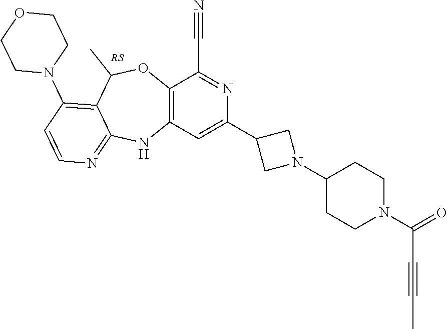 [SMILES: CC#CC(=O)N1CCC(N2CC(c3cc4c(c(C#N)n3)OC(C)c3c(N5CCOCC5)ccnc3N4)C2)CC1] |
| 512 | 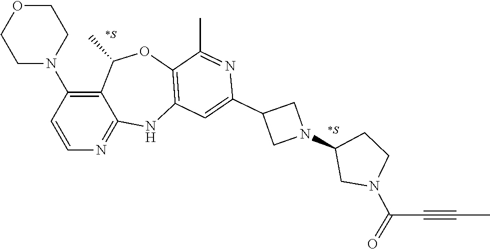 [SMILES: CC#CC(=O)N1CC[C@H](N2CC(c3cc4c(c(C)n3)O[C@@H](C)c3c(N5CCOCC5)ccnc3N4)C2)C1] |
| 513 | 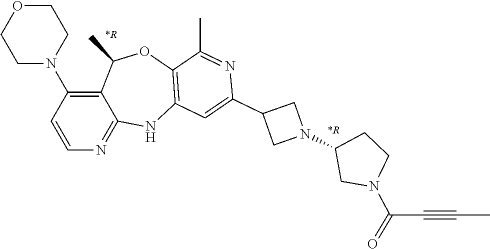 [SMILES: CC#CC(=O)N1CC[C@@H](N2CC(c3cc4c(c(C)n3)O[C@H](C)c3c(N5CCOCC5)ccnc3N4)C2)C1] |
| 514 | 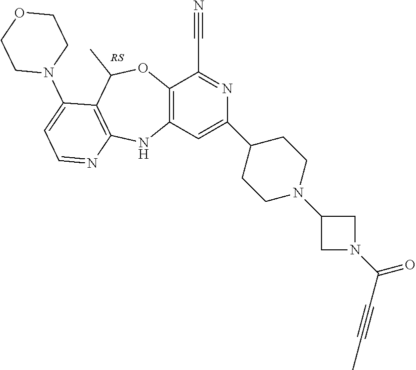 [SMILES: CC#CC(=O)N1CC(N2CCC(c3cc4c(c(C#N)n3)OC(C)c3c(N5CCOCC5)ccnc3N4)CC2)C1] |
| 515 | 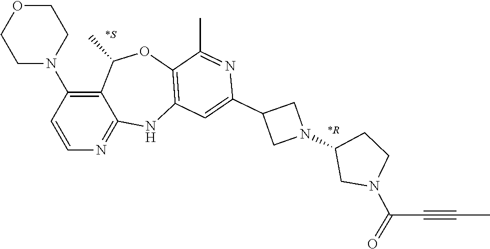 [SMILES: CC#CC(=O)N1CC[C@@H](N2CC(c3cc4c(c(C)n3)O[C@@H](C)c3c(N5CCOCC5)ccnc3N4)C2)C1] |
| 516 | 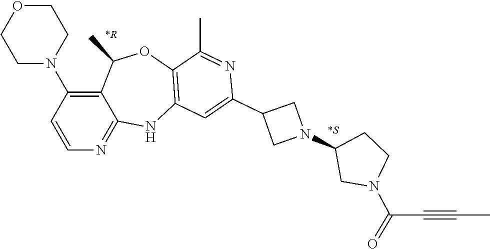 [SMILES: CC#CC(=O)N1CC[C@H](N2CC(c3cc4c(c(C)n3)O[C@H](C)c3c(N5CCOCC5)ccnc3N4)C2)C1] |
| 517 | 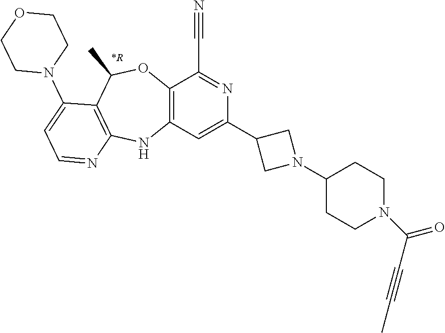 [SMILES: CC#CC(=O)N1CCC(N2CC(c3cc4c(c(C#N)n3)O[C@H](C)c3c(N5CCOCC5)ccnc3N4)C2)CC1] |
| 518 | 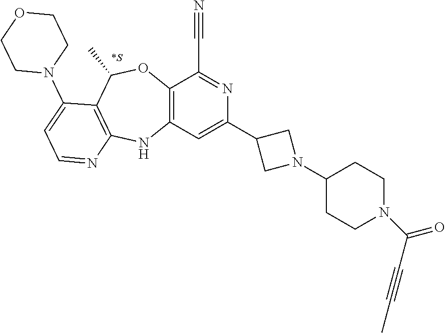 [SMILES: CC#CC(=O)N1CCC(N2CC(c3cc4c(c(C#N)n3)O[C@@H](C)c3c(N5CCOCC5)ccnc3N4)C2)CC1] |
| 519 | 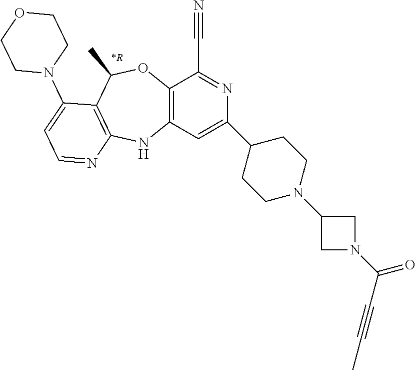 [SMILES: CC#CC(=O)N1CC(N2CCC(c3cc4c(c(C#N)n3)O[C@H](C)c3c(N5CCOCC5)ccnc3N4)CC2)C1] |
| 520 | 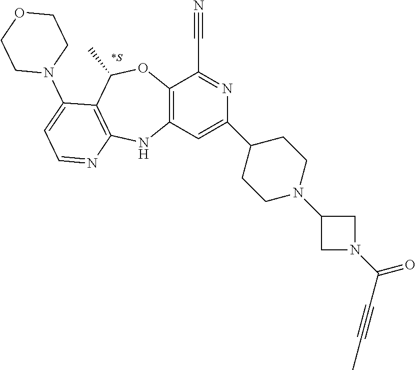 [SMILES: CC#CC(=O)N1CC(N2CCC(c3cc4c(c(C#N)n3)O[C@@H](C)c3c(N5CCOCC5)ccnc3N4)CC2)C1] |
| 521 | 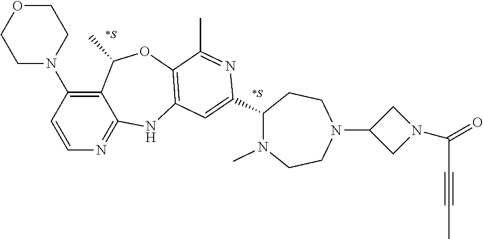 [SMILES: CC#CC(=O)N1CC(N2CC[C@@H](c3cc4c(c(C)n3)O[C@@H](C)c3c(N5CCOCC5)ccnc3N4)N(C)CC2)C1] |
| 522 | 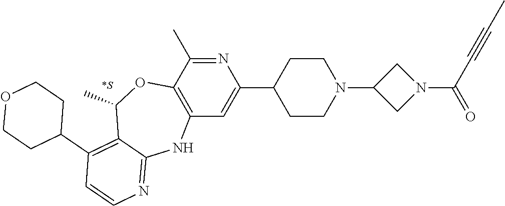 [SMILES: CC#CC(=O)N1CC(N2CCC(c3cc4c(c(C)n3)O[C@@H](C)c3c(C5CCOCC5)ccnc3N4)CC2)C1] |
| 523 | 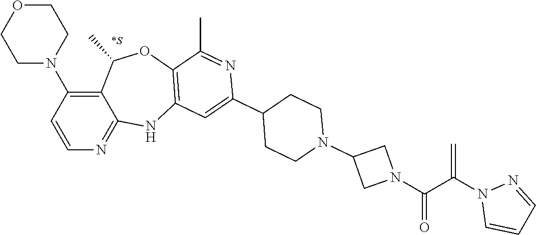 [SMILES: C=C(C(=O)N1CC(N2CCC(c3cc4c(c(C)n3)O[C@@H](C)c3c(N5CCOCC5)ccnc3N4)CC2)C1)n1cccn1] |
| 524 | 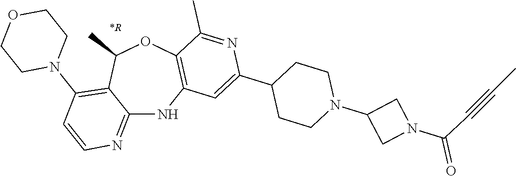 [SMILES: CC#CC(=O)N1CC(N2CCC(c3cc4c(c(C)n3)O[C@H](C)c3c(N5CCOCC5)ccnc3N4)CC2)C1] |
| 525 | 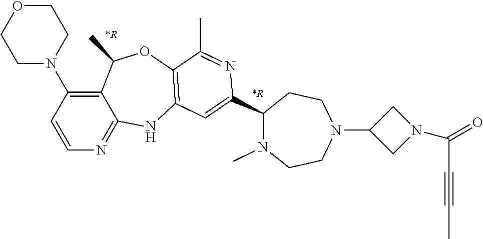 [SMILES: CC#CC(=O)N1CC(N2CC[C@H](c3cc4c(c(C)n3)O[C@H](C)c3c(N5CCOCC5)ccnc3N4)N(C)CC2)C1] |
| 526 | 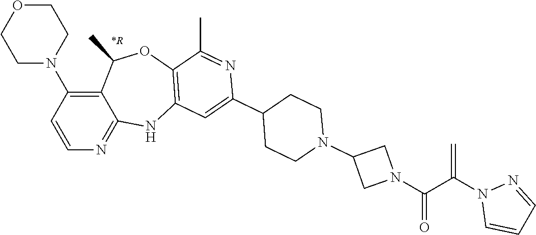 [SMILES: C=C(C(=O)N1CC(N2CCC(c3cc4c(c(C)n3)O[C@H](C)c3c(N5CCOCC5)ccnc3N4)CC2)C1)n1cccn1] |
| 527 | 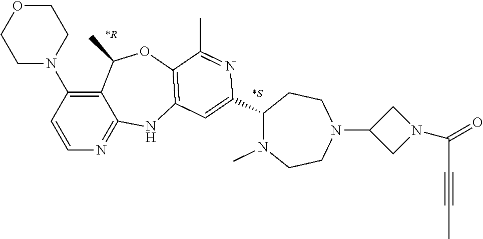 [SMILES: CC#CC(=O)N1CC(N2CC[C@@H](c3cc4c(c(C)n3)O[C@H](C)c3c(N5CCOCC5)ccnc3N4)N(C)CC2)C1] |
| 528 | 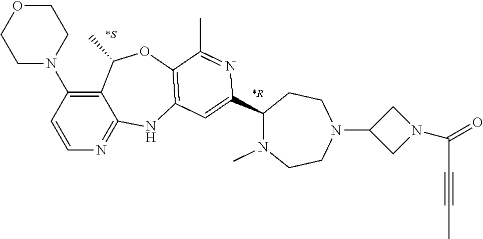 [SMILES: CC#CC(=O)N1CC(N2CC[C@H](c3cc4c(c(C)n3)O[C@@H](C)c3c(N5CCOCC5)ccnc3N4)N(C)CC2)C1] |
| 529 | 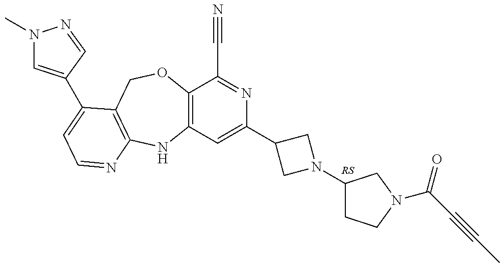 [SMILES: CC#CC(=O)N1CCC(N2CC(c3cc4c(c(C#N)n3)OCc3c(-c5cnn(C)c5)ccnc3N4)C2)C1] |
| 530 | 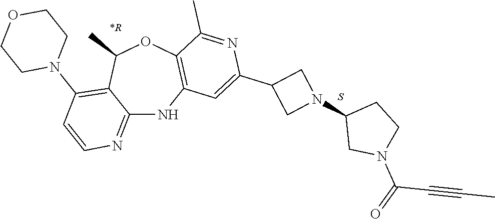 |
| 531 | 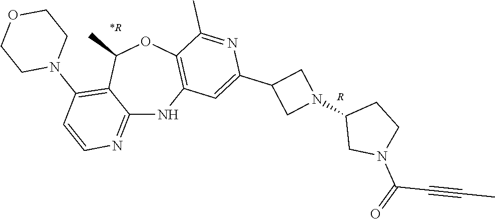 |
| 532 | 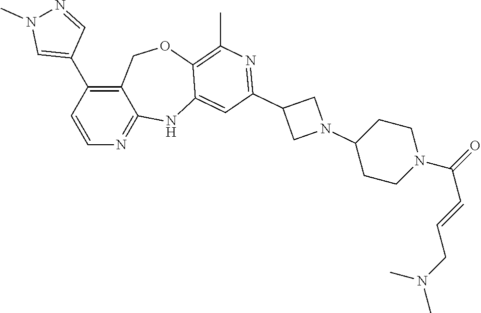 [SMILES: Cc1nc(C2CN(C3CCN(C(=O)/C=C/CN(C)C)CC3)C2)cc2c1OCc1c(-c3cnn(C)c3)ccnc1N2] |
| 533 | 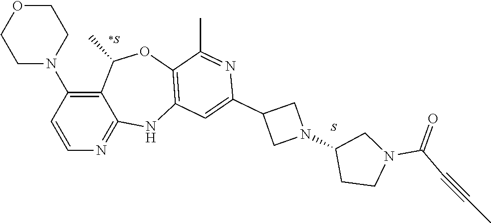 |
| 534 | 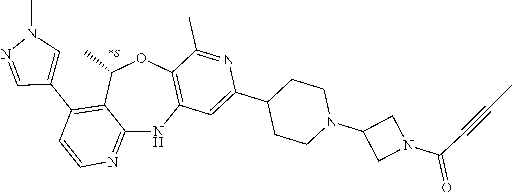 [SMILES: CC#CC(=O)N1CC(N2CCC(c3cc4c(c(C)n3)O[C@@H](C)c3c(-c5cnn(C)c5)ccnc3N4)CC2)C1] |
| 535 | 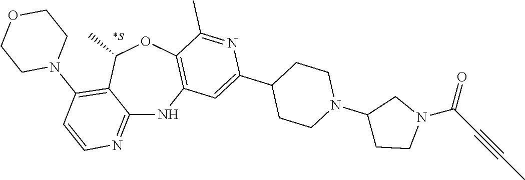 [SMILES: CC#CC(=O)N1CCC(N2CCC(c3cc4c(c(C)n3)O[C@@H](C)c3c(N5CCOCC5)ccnc3N4)CC2)C1] |
| 536 | 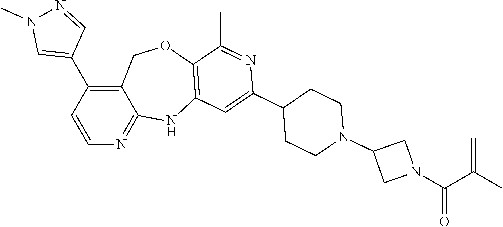 [SMILES: C=C(C)C(=O)N1CC(N2CCC(c3cc4c(c(C)n3)OCc3c(-c5cnn(C)c5)ccnc3N4)CC2)C1] |
| 537 | 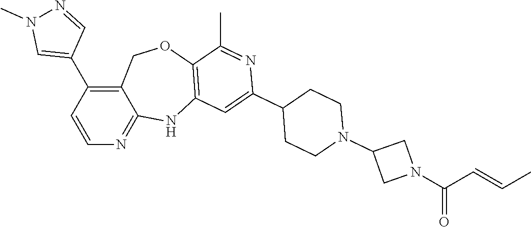 [SMILES: C/C=C/C(=O)N1CC(N2CCC(c3cc4c(c(C)n3)OCc3c(-c5cnn(C)c5)ccnc3N4)CC2)C1] |
| 538 | 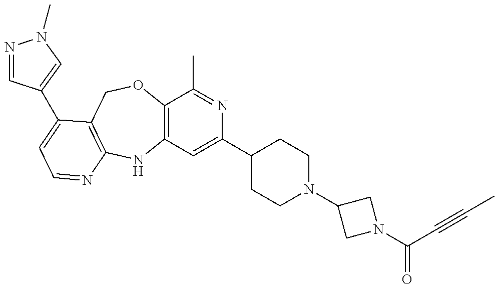 [SMILES: CC#CC(=O)N1CC(N2CCC(c3cc4c(c(C)n3)OCc3c(-c5cnn(C)c5)ccnc3N4)CC2)C1] |
| 539 | 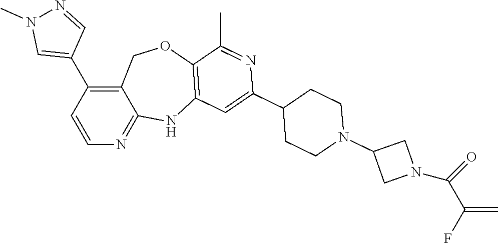 [SMILES: C=C(F)C(=O)N1CC(N2CCC(c3cc4c(c(C)n3)OCc3c(-c5cnn(C)c5)ccnc3N4)CC2)C1] |
| 540 | 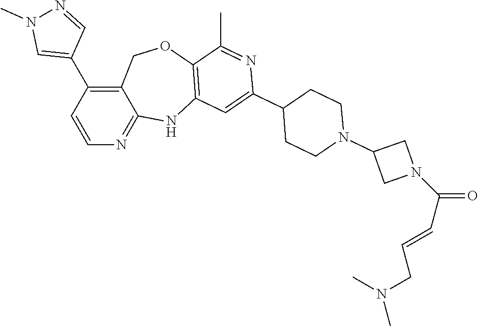 [SMILES: Cc1nc(C2CCN(C3CN(C(=O)/C=C/CN(C)C)C3)CC2)cc2c1OCc1c(-c3cnn(C)c3)ccnc1N2] |
| 541 | 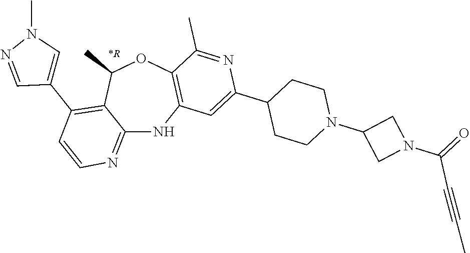 [SMILES: CC#CC(=O)N1CC(N2CCC(c3cc4c(c(C)n3)O[C@H](C)c3c(-c5cnn(C)c5)ccnc3N4)CC2)C1] |
| 542 | 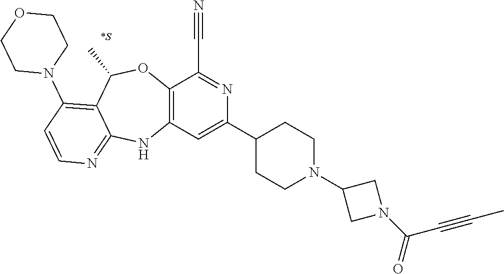 [SMILES: CC#CC(=O)N1CC(N2CCC(c3cc4c(c(C#N)n3)O[C@@H](C)c3c(N5CCOCC5)ccnc3N4)CC2)C1] |
| 543 | 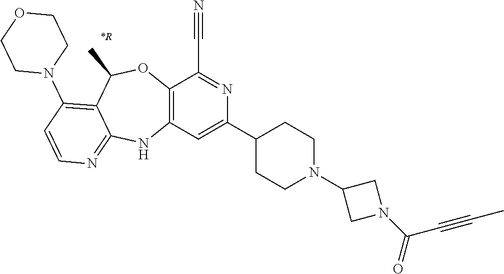 [SMILES: CC#CC(=O)N1CC(N2CCC(c3cc4c(c(C#N)n3)O[C@H](C)c3c(N5CCOCC5)ccnc3N4)CC2)C1] |
| 544 | 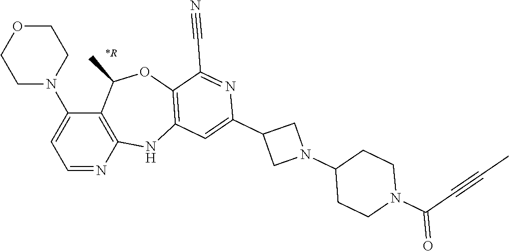 [SMILES: CC#CC(=O)N1CCC(N2CC(c3cc4c(c(C#N)n3)O[C@H](C)c3c(N5CCOCC5)ccnc3N4)C2)CC1] |
| 545 | 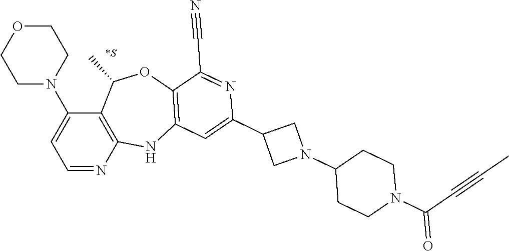 [SMILES: CC#CC(=O)N1CCC(N2CC(c3cc4c(c(C#N)n3)O[C@@H](C)c3c(N5CCOCC5)ccnc3N4)C2)CC1] |
| 546 | 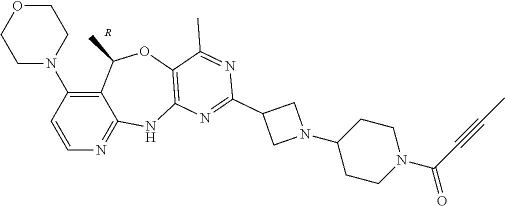 |
| 547 | 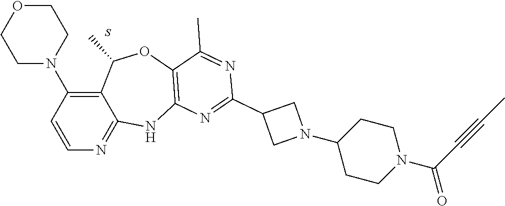 |

16. A pharmaceutical composition comprising a compound according to claim 1 and a pharmaceutically acceptable carrier.

17. (canceled)

18. (canceled)

19. (canceled)

20. (canceled)

21. (canceled)

22. A method for the treatment of a disease state or condition mediated by a CDK7, which method comprises administering to a subject in need thereof an effective amount of a compound according to claim 1.

23. The method of claim 22, wherein the disease or condition is selected from a proliferative disease, cancer, leukemia, acute myeloid leukemia (AML), chronic myelogenous leukemia (CML), lymphoma, B cell lymphoma, chronic lymphocytic leukemia (CLL), acute lymphoblastic leukemia (ALL), T-cell acute lymphoblastic leukemia (T-ALL), Hodgkin's lymphoma, non-Hodgkin's lymphoma, melanoma, multiple myeloma, bone cancer, osteosarcoma, Ewing's sarcoma, breast cancer, triple-negative breast cancer (TNBC), brain cancer, neuroblastoma, lung cancer, small cell lung cancer (SCLC), large cell lung cancer, benign neoplasm, angiogenesis, an inflammatory disease, rheumatoid arthritis, an autoinflammatory disease, or an autoimmune disease.

24. The method of claim 22, wherein the subject is a mammal.

25. An in vitro method of modulating CDK7 activity comprising contacting the CDK7 protein, or a portion thereof, with a compound according to claim 1.
